(19) United States
(12) Patent Application Publication
(10) Pub. No.: US 2011/0236903 A1
(43) Pub. Date: Sep. 29, 2011

(54) MATERIALS AND METHODS FOR DETERMINING DIAGNOSIS AND PROGNOSIS OF PROSTATE CANCER

(76) Inventors: Michael McClelland, Encinitas, CA (US); Yipeng Wang, San Diego, CA (US); Daniel Mercola, Rancho Santa Fe, CA (US)

(21) Appl. No.: 13/132,878

(22) PCT Filed: Dec. 4, 2009
(86) PCT No.: PCT/US09/66895

§ 371 Date: Jun. 3, 2011

Related U.S. Application Data
(60) Provisional application No. 61/119,996, filed on Dec. 4, 2008.

(51) Int. Cl. C12Q 1/68; C12Q 1/02; G06F 19/00

(52) U.S. Cl. 435/6.14; 435/6.1; 435/29; 702/19

(57) ABSTRACT

Materials and Methods related to diagnosing and/or determining prognosis of prostate cancer.

### CROSS-REFERENCE TO RELATED APPLICATIONS

This application claims benefit of priority from U.S. Provisional Application Ser. No. 61/119,996, filed on Dec. 4, 2008.

### STATEMENT AS TO FEDERALLY SPONSORED RESEARCH

This invention was made with government support under grant no. CA114810 awarded by the National Institutes of Health. The government has certain rights in the invention.

### TECHNICAL FIELD

This document relates to materials and methods for determining gene expression in cells, and for diagnosing prostate cancer and assessing prognosis of prostate cancer patients.

### BACKGROUND

Prostate cancer is the most common malignancy in men and is the cause of considerable morbidity and mortality (Howe et al. (2001) J. Natl. Cancer Inst. 93:824-842). It may be useful to identify genes that could be reliable early diagnostic and prognostic markers and therapeutic targets for prostate cancer, as well as other diseases and disorders.

### SUMMARY

This document is based in part on the discovery that RNA expression changes can be identified that can distinguish normal prostate stroma from tumor-adjacent stroma in the absence of tumor cells, and that such expression changes can be used to signal the “presence of tumor.” A linear regression method for the identification of cell-type specific expression of RNA from array data of prostate tumor-enriched samples was previously developed and validated (see, U.S. Publication No. 20060292572 and Stuart et al. (2004) Proc. Natl. Acad. Sci. USA 101:615-620, both incorporated herein by reference in their entirety). As described herein, the approach was extended to evaluate differential expression data obtained from normal volunteer prostate biopsy samples with tumor-adjacent stroma. Over a thousand gene expression changes were observed. A subset of stroma-specific genes were used to derive a classifier of 131 probe sets that accurately identified tumor or nontumor status of a large number of independent test cases. These observations indicate that tumor-adjacent stroma exhibits a larger number of gene expression changes and that subset may be selected to reliably identify tumor in the absence of tumor cells. The classifier may be useful in the diagnosis of stroma-rich biopsies of clinical cases with equivocal pathology readings.

The present disclosure includes, inter alia, the following: (1) extensive cross-validation of RNA biomarkers for prostate cancer relapse, across multiple datasets; (2) a “bi-modal” method for generating classifiers and testing them on samples that have mixed tissue; and (3) two methods for identifying genes in “reactive-stroma” that can be used as markers for the presence of cancer even when the sample does not include tumor but instead has regions of reactive stroma, near tumor.

In one aspect, this document features an in vitro method for identifying a subject as having or not having prostate cancer, comprising: (a) providing a prostate tissue sample from the subject; (b) measuring the level of expression for prostate cancer signature genes in the sample; (c) comparing the measured expression levels to reference expression levels for the prostate cancer signature genes; and (d) if the measured expression levels are significantly greater or less than the reference expression levels, identifying the subject as having prostate cancer, and if the measured expression levels are not significantly greater or less than the reference expression levels, identifying the subject as not having prostate cancer. The prostate tissue sample may not include tumor cells, or the prostate tissue sample may include tumor cells and stromal cells. The prostate cancer signature genes can be selected from the genes listed in Table 3 or Table 4 herein. The method can include determining whether measured expression levels for ten or more prostate cancer signature genes are significantly greater or less than reference expression levels for the ten or more prostate cancer signature genes, and classifying the subject as having prostate cancer that is likely to relapse if the measured expression levels are significantly greater or less than the reference expression levels, or classifying the subject as having prostate cancer not likely to relapse if the measured expression levels are not significantly greater or less than the reference expression levels. The ten or more prostate cancer signature genes can be selected from the genes listed in Table 3 or Table 4 herein. The method can include determining whether measured expression levels for twenty or more prostate cancer signature genes are significantly greater or less than reference expression levels for the twenty or more prostate cancer signature genes, and classifying the subject as having prostate cancer that is likely to relapse if the measured expression levels are significantly greater or less than the reference expression levels, or classifying the subject as having prostate cancer not likely to relapse if the measured expression levels are not significantly greater or less than the reference expression levels. The twenty or more prostate cancer signature genes can be selected from the genes listed in Table 3 or Table 4 herein.

In another aspect, this document features a method for determining the prognosis of a subject diagnosed as having prostate cancer, comprising: (a) providing a prostate tissue sample from the subject; (b) measuring the level of expression for prostate cancer signature genes in the sample; (c) comparing the measured expression levels to reference expression levels for the prostate cancer signature genes; and (d) if the measured expression levels are not significantly greater or less than the reference expression levels, identifying the subject as having a relatively better prognosis than if the measured expression levels are significantly greater or less than the reference expression levels, or if the measured expression levels are significantly greater or less than the reference expression levels, identifying the subject as having a relatively worse prognosis than if the measured expression levels are not significantly greater or less than the reference expression levels. The prostate tissue sample may not include tumor cells, or the prostate tissue sample may include tumor cells and stromal cells. The prostate cancer signature genes can be selected from the genes listed in Table 8A or 8B herein.

In another aspect, this document features a method for identifying a subject as having or not having prostate cancer, comprising: (a) providing a prostate tissue sample from the subject, wherein the sample comprises prostate stromal cells; (b) measuring expression levels for one or more genes in the stromal cells, wherein the one or more genes are prostate cancer signature genes; (c) comparing the measured expression levels to reference expression levels for the one or more genes, wherein the reference expression levels are determined in stromal cells from non-cancerous prostate tissue; and (d) if the measured expression levels are significantly greater or less than the reference expression levels, identifying the subject as having prostate cancer, and if the measured expression levels are not significantly greater or less than the reference expression levels, identifying the subject as not having prostate cancer. The prostate tissue sample may not include tumor cells, or the prostate tissue sample may include tumor cells and stromal cells. The prostate cancer signature genes can be selected from the genes listed in Table 3 or Table 4 herein.

In another aspect, this document features a method for determining a prognosis for a subject diagnosed as having prostate cancer, comprising: (a) providing a prostate tissue sample from the subject, wherein the sample comprises prostate stromal cells; (b) measuring expression levels for one or more genes in the stromal cells, wherein the one or more genes are prostate cancer signature genes; (c) comparing the measured expression levels to reference expression levels for the one or more genes, wherein the reference expression levels are determined in stromal cells from non-cancerous prostate tissue; and (d) if the measured expression levels are not significantly greater or less than the reference expression levels, identifying the subject as having a relatively better prognosis than if the measured expression levels are significantly greater or less than the reference expression levels, or if the measured expression levels are significantly greater or less than the reference expression levels, identifying the subject as having a relatively worse prognosis than if the measured expression levels are not significantly greater or less than the reference expression levels. The prostate tissue sample may not include tumor cells, or the prostate tissue sample may include tumor cells and stromal cells. The prostate cancer signature genes can be selected from the genes listed in Table 3 or Table 4 herein.

In still another aspect, this document features a method for identifying a subject as having or not having prostate cancer, comprising: (a) providing a prostate tissue sample from the subject; (b) measuring expression levels for one or more prostate cell-type predictor genes in the sample; (c) determining the percentages of tissue types in the sample based on the measured expression levels; (d) measuring expression levels for one more prostate cancer signature genes in the sample; (e) determining a classifier based on the percentages of tissue types and the measured expression levels; and (f) if the classifier falls into a predetermined range of prostate cancer classifiers, identifying the subject as having prostate cancer, or if the classifier does not fall into the predetermined range, identifying the subject as not having prostate cancer. Steps (b) and (d) can be carried out simultaneously.

This document also features a method for determining a prognosis for a subject diagnosed with and treated for prostate cancer, comprising: (a) providing a prostate tissue sample from the subject; (b) measuring expression levels for one or more prostate tissue predictor genes in the sample; (c) determining the percentages of tissue types in the sample based on the measured expression levels; (d) measuring expression levels for one more prostate cancer signature genes in the sample; (e) determining a classifier based on the percentages of tissue types and the measured expression levels; and (f) if the classifier falls into a predetermined range of prostate cancer relapse classifiers, identifying the subject as being likely to relapse, or if the classifier does not fall into the predetermined range, identifying the subject as not being likely to relapse. Steps (b) and (d) are carried out simultaneously.

In yet another aspect, this document features a method for identifying the proportion of two or more tissue types in a tissue sample, comprising: (a) using a set of other samples of known tissue proportions from a similar anatomical location as the tissue sample in an animal or plant, wherein at least two of the other samples do not contain the same relative content of each of the two or more cell types; (b) measuring overall levels of one or more gene expression or protein analytes in each of the other samples; (c) determining the regression relationship between the relative proportion of each tissue type and the measured overall levels of each gene expression or protein analyte in the other samples; (d) selecting one or more analytes that correlate with tissue proportions in the other samples; (e) measuring overall levels of one or more of the analytes in step (d) in the tissue sample; (f) matching the level of each analyte in the tissue sample with the level of the analyte in step (d) to determine the predicted proportion of each tissue type in the tissue sample; and (g) selecting among predicted tissue proportions for the tissue sample obtained in step (f) using either the median or average proportions of all the estimates. The tissue sample can contain cancer cells (e.g., prostate cancer cells).

In another aspect, this document features a method for comparing the levels of two or more analytes predicted by one or more methods to be associated with a change in a biological phenomenon in two sets of data each containing more than one measured sample, comprising: (a) selecting only analytes that are assayed in both sets of data; (b) ranking the analytes in each set of data using a comparative method such as the highest probability or lowest false discovery rate associated with the change in the biological phenomenon; (c) comparing a set of analytes in each ranked list in step (b) with each other, selecting those that occur in both lists, and determining the number of analytes that occur in both lists and show a change in level associated with the biological phenomenon that is in the same direction; and (d) calculating a concordance score based on the probability that the number of comparisons would show the observed number of change in the same direction, at random. In step (a), the length of each list can be varied to determine the maximum concordance score for the two ranked lists.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1A a graph plotting the incidence numbers of 339 probe sets obtained by 105-fold permutation procedure for gene selection, as described in Example 1 herein. The dashed horizontal line marks the incidence number=50. All probe sets with an incidence of >50 were selected for training using PAM using all 15 normal biopsy and the 13 original minimum tumor-bearing stroma cases. FIGS. 1B-1E are a series of histograms plotting tumor percentage for Datasets 1-4, respectively. The tumor percentage data of FIGS. 1B and 1C were provided by SPECS pathologists, while the tumor percentage data of FIGS. 1D and 1E were estimated using CellPred. Asterisks in FIG. 1B indicate misclassified tumor-bearing cases in Dataset 1.

FIG. 2A is a Venn diagram of genes identified by differential expression analysis. “b,” “t” and “a” in the plot represent normal biopsies, tumor-adjacent stroma, and rapid autopsies, respectively. FIG. 2B is a scatter plot showing differential expression of 160 probe sets in stroma cells and tumor cells. FIG. 2C is a PCA plot for a training set based on 131 selected diagnostic probe sets.

FIGS. 3A-3D are a series of scatter plots of predicted tissue percentages and pathologist estimated tissue percentages as described in Example 2 herein. X-axes: predicted tissue percentages; y-axes: pathologist estimated tissue percentages. FIG. 3A—Prediction of dataset 2 tumor percentages using models developed from dataset 1. FIG. 3B—Prediction of dataset 2 stroma percentages using models developed from dataset 1. FIG. 3C—Prediction of dataset 1 tumor percentages using models developed from dataset 2. FIG. 3D—Prediction of dataset 1 stroma percentages using models developed from dataset 2.

FIG. 4 is a series of graphs plotting predicted tissue percentages for dataset 3, as described in Example 2 herein. FIGS. 4A and 4B are histograms of predicted tumor percentages, and FIG. 4C is a plot of percentages of tumor+stroma for each individual sample.

FIG. 5 is a series of scatter plots of the differential intensity of specific genes identified as being differentially expressed between relapse and non-relapse cases found among datasets 1, 2, and 3, as described in Example 2 herein. X-axes: relapse vs. non-relapse intensity changes in dataset 1. Y-axes: relapse vs. non-relapse changes in dataset 3 (FIGS. 5A and 5B) or dataset 2 (FIG. 5C). FIG. 5A—Tumor specific genes correlating with relapse common to datasets 1 and 3. FIG. 5B—Stroma specific genes correlating with relapse common to datasets 1 and 3. FIG. 5C—Tumor specific genes correlating with relapse common to datasets 1 and 2.

FIG. 6 is a pair of graphs plotting average prediction error rates for in silico tissue component prediction discrepancies compared to pathologists' estimates using 10-fold cross validation. Solid circles: dataset 1; empty circles: dataset 2; empty squares: dataset 3; empty diamonds: dataset 4. X-axes: number of genes used in the prediction model. Y-axes: average prediction error rates (%). FIG. 6A shows prediction error rates for tumor components, and FIG. 6B shows prediction error rates for stroma components.

FIG. 7 is a pair of graphs showing tissue component predictions on publicly available datasets. FIG. 7A is a histogram plot of the in silico predicted tumor components (%) of 219 arrays that were generated from samples prepared as tumor-enriched prostate cancer samples. X-axis: in silico predicted tumor cell percentages (%). Y-axis: frequency of samples. FIG. 7B is a box-plot showing the differences of tumor tissue components in non-recurrence and recurrence groups of prostate cancer samples for dataset 5. X-axis: sample groups, NR: non-recurrence group; REC: recurrence group. Y-axis: tumor cell percentages (%).

FIG. 8 is a series of scatter plots showing predicted tissue percentages and pathologist estimated tissue percentages. X-axis: predicted tissue percentages; y-axis: pathologist estimated tissue percentages. FIG. 8A—Prediction of dataset 2 tumor percentages using models developed from dataset 1. The Pearson correlation coefficient is 0.74. FIG. 8B—Prediction of dataset 2 stroma percentages using models developed from dataset 1. The Pearson correlation coefficient is 0.70. FIG. 8C—Prediction of dataset 2 BPH percentages using models developed from dataset 1. The Pearson correlation coefficient is 0.45. FIG. 8D—Prediction of dataset 1 tumor percentages using models developed from dataset 2. The Pearson Correlation Coefficient is 0.87. FIG. 8E—Prediction of dataset 1 stroma percentages using models developed from dataset 2. The Pearson Correlation Coefficient is 0.78. FIG. 8F—Prediction of dataset 1 BPH percentages using models developed from dataset 2. The Pearson Correlation Coefficient is 0.57.

FIG. 9 is a pair of graphs plotting correlation of the amount of differential gene expression, termed gamma, between disease recurrence and disease free cases for a 91 patient case set measured on U133A GeneChips compared to an independent 86 patient case set measured on the U133A plus2 platform. Genes are identified as specific to differential expression by tumor epithelial cells, “gamma T,” left panel, or stroma cells, “gamma S,” right panel.

FIG. 10 is a graph plotting correlation between the quantification of stain concentration between a trained human expert and the proposed unsupervised method. Circles represent individual scores for a given tissue sample (a total of 97 samples). The line is result of unsupervised spectral unmixing for concentration estimation. The unsupervised approach is within 3% of the linear regression of the manually labeled data.

FIG. 11 is a flow diagram of the automated acquisition and visualization demonstrated on a colon cancer tissue microarray. The only inputs required are the scan area (x, y, dx, dy) and the number of cores. After these steps are completed, the images are ready for diagnosis/scoring. The image in “b” is a single field of view from a 20× objective and “c” is a montage of images acquired at 20×.

FIG. 12 is a graph plotting genes identified when different sample sizes were used (circles). The squares represent the overlap between the longest gene list (666 genes at sample size=120) and other gene lists. The other points (s and t) illustrate the overlap between each gene lists and the tumor/stroma genes identified with MLR.

FIGS. 13A and 13B are graphs representing relapse associated genes identified for tumor cells, while FIGS. 13C-13F show relapse associated genes identified for stroma cells. The circles indicate the numbers of genes identified when different sample sizes were used. The squares represent the overlap between the reference gene list and other gene lists. The other points illustrate the overlap between each gene lists and the tumor/stroma genes identified with MLR.

FIG. 14 is a graph plotting results by averaging 100 randomly selected samples when different sample sizes were used for differential expression analysis. The squares, circles, and diamonds represent specificity, sensitivity and false discovery rate, respectively.

### DETAILED DESCRIPTION

Unless defined otherwise, all technical and scientific terms used herein have the same meaning as is commonly understood by one of skill in the art to which the invention(s) belong. All patents, patent applications, published applications and publications, GENBANK® sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it understood that such identifiers particular information on the internet can change, equivalent information can be found by searching the internet. Reference thereto evidences the availability and public dissemination of such information.

Differential expression includes to both quantitative as well as qualitative differences in the extend of the genes' expression depending on differential development and/or tumor growth. Differentially expressed genes can represent marker genes, and/or target genes. The expression pattern of a differentially expressed gene disclosed herein can be utilized as part of a prognostic or diagnostic evaluation of a subject. The expression pattern of a differentially expressed gene can be used to identify the presence of a particular cell type in a sample. A differentially expressed gene disclosed herein can be used in methods for identifying reagents and compounds and uses of these reagents and compounds for the treatment of a subject as well as methods of treatment.

The terms “biological activity,” “bioactivity,” “activity,” and “biological function” can be used interchangeably, and can refer to an effector or antigenic function that is directly or indirectly performed by a polypeptide (whether in its native or denatured conformation), or by any fragment thereof in vivo or in vitro. Biological activities include, without limitation, binding to polypeptides, binding to other proteins or molecules, enzymatic activity, signal transduction, activity as a DNA binding protein, as a transcription regulator, and ability to bind damaged DNA. A bioactivity can be modulated by directly affecting the subject polypeptide. Alternatively, a bioactivity can be altered by modulating the level of the polypeptide, such as by modulating expression of the corresponding gene.

The term “gene expression analyte” refers to a biological molecule whose presence or concentration can be detected and correlated with gene expression. For example, a gene expression analyte can be a mRNA of a particular gene, or a fragment thereof (including, e.g., by-products of mRNA splicing and nucleolytic cleavage fragments), a protein of a particular gene or a fragment thereof (including, e.g., post-translationally modified proteins or by-products therefrom, and proteolytic fragments), and other biological molecules such as a carbohydrate, lipid or small molecule, whose presence or absence corresponds to the expression of a particular gene.

A gene expression level is to the amount of biological macromolecule produced from a gene. For example, expression levels of a particular gene can refer to the amount of protein produced from that particular gene, or can refer to the amount of mRNA produced from that particular gene. Gene expression levels can refer to an absolute (e.g., molar or gram-quantity) levels or relative (e.g., the amount relative to a standard, reference, calibration, or to another gene expression level). Typically, gene expression levels used herein are relative expression levels. As used herein in regard to determining the relationship between cell content and expression levels, gene expression levels can be considered in terms of any manner of describing gene expression known in the art. For example, regression methods that consider gene expression levels can consider the measurement of the level of a gene expression analyte, or the level calculated or estimated according to the measurement of the level of a gene expression analyte.

A marker gene is a differentially expressed gene which expression pattern can serve as part of a phenotype-indicating method, such as a predictive method, prognostic or diagnostic method, or other cell-type distinguishing evaluation, or which, alternatively, can be used in methods for identifying compounds useful for the treatment or prevention of diseases or disorders, or for identifying compounds that modulate the activity of one or more gene products.

A phenotype indicated by methods provided herein can be a diagnostic indication, a prognostic indication, or an indication of the presence of a particular cell type in a subject. Diagnostic indications include indication of a disease or a disorder in the subject, such as presence of tumor or neoplastic disease, inflammatory disease, autoimmune disease, and any other diseases known in the art that can be identified according to the presence or absence of particular cells or by the gene expression of cells. In another embodiment, prognostic indications refers to the likely or expected outcome of a disease or disorder, including, but not limited to, the likelihood of survival of the subject, likelihood of relapse, aggressiveness of the disease or disorder, indolence of the disease or disorder, and likelihood of success of a particular treatment regimen.

The phrase “gene expression levels that correspond to levels of gene expression analytes” refers to the relationship between an analyte that indicates the expression of a gene, and the actual level of expression of the gene. Typically the level of a gene expression analyte is measured in experimental methods used to determine gene expression levels. As understood by one skilled in the art, the measured gene expression levels can represent gene expression at a variety of levels of detail (e.g., the absolute amount of a gene expressed, the relative amount of gene expressed, or an indication of increased or decreased levels of expression). The level of detail at which the levels of gene expression analytes can indicate levels of gene expression can be based on a variety of factors that include the number of controls used, the number of calibration experiments or reference levels determined, and other factors known in the art. In some methods provided herein, increase in the levels of a gene expression analyte can indicate increase in the levels of the gene expressed, and a decrease in the levels of a gene expression analyte can indicate decrease in the levels of the gene expressed.

A regression relationship between relative content of a cell type and measured overall levels of a gene expression analyte is a quantitative relationship between cell type and level of gene expression analyte that is determined according to the methods provided herein based on the amount of cell type present in two or more samples and experimentally measured levels of gene expression analyte. In one embodiment, the regression relationship is determined by determining the regression of overall levels of each gene expression analyte on determined cell proportions. In one embodiment, the regression relationship is determined by linear regression, where the overall expression level or the expression analyte level is treated as directly proportional to (e.g., linear in) cell percent either for each cell type in turn or all at once and the slopes of these linear relationships can be expressed as beta values.

As used herein, a heterogeneous sample is to a sample that contains more than one cell type. For example, a heterogeneous sample can contain stromal cells and tumor cells. Typically, as used herein, the different cell types present in a sample are present in greater than about 0.1%, 0.2%, 0.3%, 0.5%, 0.7%, 1%, 2%, 3%, 4% or 5% or greater than 0.1%, 0.2%, 0.3%, 0.5%, 0.7%, 1%, 2%, 3%, 4% or 5%. As is understood in the art, cell samples, such as tissue samples from a subject, can contain minute amounts of a variety of cell types (e.g., nerve, blood, vascular cells). However, cell types that are not present in the sample in amounts greater than about 0.1%, 0.2%, 0.3%, 0.5%, 0.7%, 1%, 2%, 3%, 4% or 5% or greater than 0.1%, 0.2%, 0.3%, 0.5%, 0.7%, 1%, 2%, 3%, 4% or 5%, are not typically considered components of the heterogeneous cell sample, as used herein.

Related cell samples can be samples that contain one or more cell types in common. Related cell samples can be samples from the same tissue type or from the same organ. Related cell samples can be from the same or different sources (e.g., same or different individuals or cell cultures, or a combination thereof). As provided herein, in the case of three or more different cell samples, it is not required that all samples contain a common cell type, but if a first sample does not contain any cell types that are present in the other samples, the first sample is not related to the other samples.

Tumor cells are cells with cytological and adherence properties consisting of nuclear and cyoplasmic features and patterns of cell-to-cell association that are known to pathologists skilled in the art as sufficient for the diagnosis as cancers of various types. In some embodiments, tumor cells have abnormal growth properties, such as neoplastic growth properties.

The “cells associated with tumor” refers to cells that, while not necessarily malignant, are present in tumorous tissues or organs or particular locations of tissues or organs, and are not present, or are present at insignificant levels, in normal tissues or organs, or in particular locations of tissues or organs.

Benign prostatic hyperplastic (BPH) cells are cells of the epithelial lining of hyperplastic prostate glands. Dilated cystic glands cells are cells of the epithelial lining of dilated (atrophic) cystic prostate glands.

Stromal cells include connective tissue cells and smooth muscle cells forming the stroma of an organ. Exemplary stromal cells are cells of the stroma of the prostate gland.

A reference refers to a value or set of related values for one or more variables. In one example, a reference gene expression level refers to a gene expression level in a particular cell type. Reference expression levels can be determined according to the methods provided herein, or by determining gene expression levels of a cell type in a homogenous sample. Reference levels can be in absolute or relative amounts, as is known in the art. In certain embodiments, a reference expression level can be indicative of the presence of a particular cell type. For example, in certain embodiments, only one particular cell type may have high levels of expression of a particular gene, and, thus, observation of a cell type with high measured expression levels can match expression levels of that particular cell type, and thereby indicate the presence of that particular cell type in the sample. In another embodiment, a reference expression level can be indicative of the absence of a particular cell type. As provided herein, two or more references can be considered in determining whether or not a particular cell type is present in a sample, and also can be considered in determining the relative amount of a particular cell type that is present in the sample.

A modified t statistic is a numerical representation of the ability of a particular gene product or indicator thereof to indicate the presence or absence of a particular cell type in a sample. A modified t statistic incorporating goodness of fit and effect size can be formulated according to known methods (see, e.g., Tusher (2001) Proc. Natl. Acad. Sci. USA 98:5116-5121), where σ_{β} is the standard error of the coefficient, and k is a small constant, as follows:

t=β/(k+σ_{β})

The relative content of a cell type or cell proportion is the amount of a cell mixture that is populated by a particular cell type. Typically, heterogeneous cell mixtures contain two or more cell types, and, therefore, no single cell type makes up 100% of the mixture. Relative content can be expressed in any of a variety of forms known in the art; For example, relative content can be expressed as a percentage of the total amount of cells in a mixture, or can be expressed relative to the amount of a particular cell type. As used herein, percent cell or percent cell composition is the percent of all cells that a particular cell type accounts for in a heterologous cell mixture, such as a microscopic section sampling a tissue.

An array or matrix is an arrangement of addressable locations or addresses on a device. The locations can be arranged in two dimensional arrays, three dimensional arrays, or other matrix formats. The number of locations can range from several to at least hundreds of thousands. Most importantly, each location represents a totally independent reaction site. Arrays include but are not limited to nucleic acid arrays, protein arrays and antibody arrays. A nucleic acid array refers to an array containing nucleic acid probes, such as oligonucleotides, polynucleotides or larger portions of genes. The nucleic acid on the array can be single stranded. Arrays wherein the probes are oligonucleotides are referred to as oligonucleotide arrays or oligonucleotide chips. A microarray, herein also refers to a biochip or biological chip, an array of regions having a density of discrete regions of at least about 100/cm², and can be at least about 1000/cm². The regions in a microarray have typical dimensions, e.g., diameters, in the range of between about 10-250 μm, and are separated from other regions in the array by about the same distance. A protein array refers to an array containing polypeptide probes or protein probes which can be in native form or denatured. An antibody array refers to an array containing antibodies which include but are not limited to monoclonal antibodies (e.g., from a mouse), chimeric antibodies, humanized antibodies or phage antibodies and single chain antibodies as well as fragments from antibodies.

An agonist is an agent that mimics or upregulates (e.g., potentiates or supplements) the bioactivity of a protein. An agonist can be a wild-type protein or derivative thereof having at least one bioactivity of the wild-type protein. An agonist can also be a compound that upregulates expression of a gene or which increases at least one bioactivity of a protein. An agonist can also be a compound which increases the interaction of a polypeptide with another molecule, e.g., a target peptide or nucleic acid.

The terms “polynucleotide” and “nucleic acid molecule” refer to nucleotides of any length, either ribonucleotides or deoxyribonucleotides. This term refers only to the primary structure of the molecule. Thus, this term includes double- and single-stranded DNA and RNA. It also includes known types of modifications, for example, labels which are known in the art, methylation, caps, substitution of one or more of the naturally occurring nucleotides with an analog, internucleotide modifications such as, for example, those with uncharged linkages (e.g., phosphorothioates and phosphorodithioates), those containing pendant moieties, such as, for example, proteins (including, e.g., nucleases, toxins, antibodies, signal peptides, and poly-L-lysine), those with intercalators (e.g., acridine and psoralen), those containing chelators (e.g., metals and radioactive metals), those containing alkylators, those with modified linkages (e.g., alpha anomeric nucleic acids), and those containing nucleotide analogs (e.g., peptide nucleic acids), as well as unmodified forms of the polynucleotide.

A polynucleotide derived from a designated sequence typically is a polynucleotide sequence which is comprised of a sequence of approximately at least about 6 nucleotides, at least about 8 nucleotides, at least about 10-12 nucleotides, or at least about 15-20 nucleotides corresponding to a region of the designated nucleotide sequence. Corresponding polynucleotides are homologous to or complementary to a designated sequence. Typically, the sequence of the region from which the polynucleotide is derived is homologous to or complementary to a sequence that is unique to a gene provided herein.

Recombinant polypeptides are polypeptides made using recombinant techniques, i.e., through the expression of a recombinant nucleic acid. A recombinant polypeptide can be distinguished from naturally occurring polypeptide by at least one or more characteristics. For example, the polypeptide may be isolated or purified away from some or all of the proteins and compounds with which it is normally associated in its wild type host, and thus may be substantially pure. For example, an isolated polypeptide is unaccompanied by at least some of the material with which it is normally associated in its natural state, constituting at least about 0.5%, or at least about 5% by weight of the total protein in a given sample. A substantially pure polypeptide comprises at least about 50-75% by weight of the total protein, at least about 80%, or at least about 90%. The definition includes the production of a polypeptide from one organism in a different organism or host cell. Alternatively, the polypeptide may be made at a significantly higher concentration than is normally seen, through the use of an inducible promoter or high expression promoter, such that the protein is made at increased concentration levels. Alternatively, the polypeptide may be in a form not normally found in nature, as in the addition of an epitope tag or amino acid substitutions, insertions and deletions, as discussed below.

The terms “disease” and “disorder” refer to a pathological condition in an organism resulting from, e.g., infection or genetic defect, and characterized by identifiable symptoms.

The “percent sequence identity” between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained from Fish & Richardson's web site (world wide web at fr.com/blast) or the United States government's National Center for Biotechnology Information web site (world wide web at ncbi.nlm.nih.gov). Instructions explaining how to use the B12seq program can be found in the readme file accompanying BLASTZ. B12seq performs a comparison between two sequences using either the BLASTN or BLASTP algorithm BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: CAB12seq-i c:\seq1.txt-j c:\seq2.txt-p blastn-o c:\output.txt-q−1-r 2. To compare two amino acid sequences, the options of B12seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq-i c:\seq1.txt-j c:\seq2.txt-p blastp-o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (e.g., 100 consecutive nucleotides or amino acid residues from a sequence set forth in an identified sequence), followed by multiplying the resulting value by 100. For example, a nucleic acid sequence that has 1166 matches when aligned with a 1200 bp sequence is 97.1 percent identical to the 1200 bp sequence (i.e., 1166÷1200*100=97.1). It is noted that the percent sequence identity value is rounded to the nearest tenth. For example, 75.11, 75.12, 75.13, and 75.14 is rounded down to 75.1, while 75.15, 75.16, 75.17, 75.18, and 75.19 is rounded up to 75.2. It is also noted that the length value will always be an integer. In another example, a target sequence containing a 20-nucleotide region that aligns with 20 consecutive nucleotides from an identified sequence as follows contains a region that shares 75 percent sequence identity to that identified sequence (i.e., 15÷20*100=75). Polypeptides that at least 90% identical have percent identities from 90 to 100 relative to the reference polypeptides. Identity at a level of 90% or more can be indicative of the fact that, for a polynucleotide length of 100 amino acids no more than 10% (i.e., 10 out of 100) amino acids in the test polypeptide differ from those of the reference polypeptides. Similar comparisons can be made between test and reference polynucleotides. Such differences can be represented as point mutations randomly distributed over the entire length of an amino acid sequence or they can be clustered in one or more locations of varying length up to the maximum allowable, e.g., 10/100 amino acid difference (approximately 90% identity). Differences are defined as nucleic acid or amino acid substitutions, or deletions. At the level of homologies or identities above about 85-90%, the result should be independent of the program and gap parameters set; such high levels of identity can be assessed readily, often without relying on software.

A primer refers to an oligonucleotide containing two or more deoxyribonucleotides or ribonucleotides, typically more than three, from which synthesis of a primer extension product can be initiated. Experimental conditions conducive to synthesis include the presence of nucleoside triphosphates and an agent for polymerization and extension, such as DNA polymerase, and a suitable buffer, temperature and pH.

Animals can include any animal, such as, but are not limited to, goats, cows, deer, sheep, rodents, pigs and humans. Non-human animals, exclude humans as the contemplated animal. The SPs provided herein are from any source, animal, plant, prokaryotic and fungal.

Genetic therapy can involve the transfer of heterologous nucleic acid, such as DNA, into certain cells, target cells, of a mammal, particularly a human, with a disorder or conditions for which such therapy is sought. The nucleic acid, such as DNA, is introduced into the selected target cells in a manner such that the heterologous nucleic acid, such as DNA, is expressed and a therapeutic product encoded thereby is produced. Alternatively, the heterologous nucleic acid, such as DNA, can in some manner mediate expression of DNA that encodes the therapeutic product, or it can encode a product, such as a peptide or RNA that in some manner mediates, directly or indirectly, expression of a therapeutic product. Genetic therapy can also be used to deliver nucleic acid encoding a gene product that replaces a defective gene or supplements a gene product produced by the mammal or the cell in which it is introduced. The introduced nucleic acid can encode a therapeutic compound, such as a growth factor inhibitor thereof, or a tumor necrosis factor or inhibitor thereof, such as a receptor therefor, that is not normally produced in the mammalian host or that is not produced in therapeutically effective amounts or at a therapeutically useful time. The heterologous nucleic acid, such as DNA, encoding the therapeutic product can be modified prior to introduction into the cells of the afflicted host in order to enhance or otherwise alter the product or expression thereof. Genetic therapy can also involve delivery of an inhibitor or repressor or other modulator of gene expression.

A heterologous nucleic acid is nucleic acid that encodes RNA or RNA and proteins that are not normally produced in vivo by the cell in which it is expressed or that mediates or encodes mediators that alter expression of endogenous nucleic acid, such as DNA, by affecting transcription, translation, or other regulatable biochemical processes. Heterologous nucleic acid, such as DNA, can also be referred to as foreign nucleic acid, such as DNA. Any nucleic acid, such as DNA, that one of skill in the art would recognize or consider as heterologous or foreign to the cell in which is expressed is herein encompassed by heterologous nucleic acid; heterologous nucleic acid includes exogenously added nucleic acid that is also expressed endogenously. Examples of heterologous nucleic acid include, but are not limited to, nucleic acid that encodes traceable marker proteins, such as a protein that confers drug resistance, nucleic acid that encodes therapeutically effective substances, such as anti-cancer agents, enzymes and hormones, and nucleic acid, such as DNA, that encodes other types of proteins, such as antibodies. Antibodies that are encoded by heterologous nucleic acid can be secreted or expressed on the surface of the cell in which the heterologous nucleic acid has been introduced. Heterologous nucleic acid is generally not endogenous to the cell into which it is introduced, but has been obtained from another cell or prepared synthetically. Generally, although not necessarily, such nucleic acid encodes RNA and proteins that are not normally produced by the cell in which it is now expressed.

A therapeutically effective product for gene therapy can be a product encoded by heterologous nucleic acid, typically DNA, that, upon introduction of the nucleic acid into a host, a product is expressed that ameliorates or eliminates the symptoms, manifestations of an inherited or acquired disease or that cures the disease. Also included are biologically active nucleic acid molecules, such as RNAi and antisense.

Disease or disorder treatment or compound can include any therapeutic regimen and/or agent that, when used alone or in combination with other treatments or compounds, can alleviate, reduce, ameliorate, prevent, or place or maintain in a state of remission of clinical symptoms or diagnostic markers associated with the disease or disorder.

Nucleic acids include DNA, RNA and analogs thereof, including peptide nucleic acids (PNA) and mixtures thereof. Nucleic acids can be single or double-stranded. When referring to probes or primers, optionally labeled, with a detectable label, such as a fluorescent or radiolabel, single-stranded molecules are contemplated. Such molecules are typically of a length such that their target is statistically unique or of low copy number (typically less than 5, generally less than 3) for probing or priming a library. Generally a probe or primer contains at least 14, 16 or 30 contiguous of sequence complementary to or identical a gene of interest. Probes and primers can be 10, 20, 30, 50, 100 or more nucleic acids long.

Operative linkage of heterologous nucleic acids to regulatory and effector sequences of nucleotides, such as promoters, enhancers, transcriptional and translational stop sites, and other signal sequences refers to the relationship between such nucleic acid, such as DNA, and such sequences of nucleotides. Thus, operatively linked or operationally associated refers to the functional relationship of nucleic acid, such as DNA, with regulatory and effector sequences of nucleotides, such as promoters, enhancers, transcriptional and translational stop sites, and other signal sequences. For example, operative linkage of DNA to a promoter refers to the physical and functional relationship between the DNA and the promoter such that the transcription of such DNA is initiated from the promoter by an RNA polymerase that specifically recognizes, binds to and transcribes the DNA. In order to optimize expression and/or in vitro transcription, it can be necessary to remove, add or alter 5′ untranslated portions of the clones to eliminate extra, potential inappropriate alternative translation initiation (i.e., start) codons or other sequences that can interfere with or reduce expression, either at the level of transcription or translation. Alternatively, consensus ribosome binding sites (see, e.g., Kozak (1991) J. Biol. Chem. 266:19867-19870) can be inserted immediately 5′ of the start codon and can enhance expression. The desirability of (or need for) such modification can be empirically determined.

A sequence complementary to at least a portion of an RNA, with reference to antisense oligonucleotides, means a sequence having sufficient complementarity to be able to hybridize with the RNA, generally under moderate or high stringency conditions, forming a stable duplex; in the case of double-stranded antisense nucleic acids, a single strand of the duplex DNA (or dsRNA) can thus be tested, or triplex formation can be assayed. The ability to hybridize depends on the degree of complementarily and the length of the antisense nucleic acid. Generally, the longer the hybridizing nucleic acid, the more base mismatches with a gene encoding RNA it can contain and still form a stable duplex (or triplex, as the case can be). One skilled in the art can ascertain a tolerable degree of mismatch by use of standard procedures to determine the melting point of the hybridized complex.

Antisense polynucleotides are synthetic sequences of nucleotide bases complementary to mRNA or the sense strand of double-stranded DNA. Admixture of sense and antisense polynucleotides under appropriate conditions leads to the binding of the two molecules, or hybridization. When these polynucleotides bind to (hybridize with) mRNA, inhibition of protein synthesis (translation) occurs. When these polynucleotides bind to double-stranded DNA, inhibition of RNA synthesis (transcription) occurs. The resulting inhibition of translation and/or transcription leads to an inhibition of the synthesis of the protein encoded by the sense strand. Antisense nucleic acid molecules typically contain a sufficient number of nucleotides to specifically bind to a target nucleic acid, generally at least 5 contiguous nucleotides, often at least 14 or 16 or 30 contiguous nucleotides or modified nucleotides complementary to the coding portion of a nucleic acid molecule that encodes a gene of interest.

An antibody is an immunoglobulin, whether natural or partially or wholly synthetically produced, including any derivative thereof that retains the specific binding ability the antibody. Hence antibody includes any protein having a binding domain that is homologous or substantially homologous to an immunoglobulin binding domain. Antibodies include members of any immunoglobulin groups, including, but not limited to, IgG, IgM, IgA, IgD, IgY and IgE.

An antibody fragment is any derivative of an antibody that is less than full-length, retaining at least a portion of the full-length antibody's specific binding ability. Examples of antibody fragments include, but are not limited to, Fab, Fab′, F(ab)₂, single-chain Fvs (scFV), FV, dsFV diabody and Fd fragments. The fragment can include multiple chains linked together, such as by disulfide bridges. An antibody fragment generally contains at least about 50 amino acids and typically at least 200 amino acids.

An Fv antibody fragment is composed of one variable heavy domain (VH) and one variable light domain linked by noncovalent interactions. A dsFV is an Fv with an engineered intermolecular disulfide bond, which stabilizes the VH-VL pair. An F(ab)₂ fragment is an antibody fragment that results from digestion of an immunoglobulin with pepsin at pH 4.0-4.5; it can be recombinantly expressed to produce the equivalent fragment.

Fab fragments are antibody fragments that result from digestion of an immunoglobulin with papain; they can be recombinantly expressed to produce the equivalent fragment.

scFVs refer to antibody fragments that contain a variable light chain (VL) and variable heavy chain (VH) covalently connected by a polypeptide linker in any order. The linker is of a length such that the two variable domains are bridged without substantial interference. Included linkers are (Gly-Ser)n residues with some Glu or Lys residues dispersed throughout to increase solubility.

Humanized antibodies are antibodies that are modified to include human sequences of amino acids so that administration to a human does not provoke an immune response. Methods for preparation of such antibodies are known. For example, to produce such antibodies, the encoding nucleic acid in the hybridoma or other prokaryotic or eukaryotic cell, such as an E. coli or a CHO cell, that expresses the monoclonal antibody is altered by recombinant nucleic acid techniques to express an antibody in which the amino acid composition of the non-variable region is based on human antibodies. Computer programs have been designed to identify such non-variable regions.

Diabodies are dimeric scFV; diabodies typically have shorter peptide linkers than scFvs, and they generally dimerize.

The phrase “production by recombinant means by using recombinant DNA methods” refers to the use of the well known methods of molecular biology for expressing proteins encoded by cloned DNA.

An “effective amount” of a compound for treating a particular disease is an amount that is sufficient to ameliorate, or in some manner reduce the symptoms associated with the disease. Such amount can be administered as a single dosage or can be administered according to a regimen, whereby it is effective. The amount can cure the disease but, typically, is administered in order to ameliorate the symptoms of the disease. Repeated administration can be required to achieve the desired amelioration of symptoms.

A compound that modulates the activity of a gene product either decreases or increases or otherwise alters the activity of the protein or, in some manner up- or down-regulates or otherwise alters expression of the nucleic acid in a cell.

Pharmaceutically acceptable salts, esters or other derivatives of the conjugates include any salts, esters or derivatives that can be readily prepared by those of skill in this art using known methods for such derivatization and that produce compounds that can be administered to animals or humans without substantial toxic effects and that either are pharmaceutically active or are prodrugs.

A drug or compound identified by the screening methods provided herein refers to any compound that is a candidate for use as a therapeutic or as a lead compound for the design of a therapeutic. Such compounds can be small molecules, including small organic molecules, peptides, peptide mimetics, antisense molecules or dsRNA, such as RNAi, antibodies, fragments of antibodies, recombinant antibodies and other such compounds that can serve as drug candidates or lead compounds.

A non-malignant cell adjacent to a malignant cell in a subject is a cell that has a normal morphology (e.g., is not classified as neoplastic or malignant by a pathologist, cell sorter, or other cell classification method), but, while the cell was present intact in the subject, the cell was adjacent to a malignant cell or malignant cells. As provided herein, cells of a particular type (e.g., stroma) adjacent to a malignant cell or malignant cells can display an expression pattern that differs from cells of the same type that are not adjacent to a malignant cell or malignant cells. In accordance with the methods provided herein, cells that are adjacent to malignant cells can be distinguished from cells of the same type that are adjacent to non-malignant cells, according to their differential gene expression. As used herein regarding the location of cells, adjacent refers to a first cell and a second cell being sufficiently proximal such that the first cell influences the gene expression of the second cell. For example, adjacent cells can include cells that are in direct contact with each other, adjacent cell can include cells within 500 microns, 300 microns, 200 microns 100 microns or 50 microns, of each other.

A tumor is a collection of malignant cells. Malignant as applied to a cell refers to a cell that grows in an uncontrolled fashion. In some embodiments, a malignant cell can be anaplastic. In some embodiments, a malignant cell can be capable of metastasizing.

Hybridization stringency for, which can be used to determine percentage mismatch is as follows:

1) high stringency: 0.1×SSPE, 0.1% SDS, 65° C.

2) medium stringency: 0.2×SSPE, 0.1% SDS, 50° C.

3) low stringency: 1.0×SSPE, 0.1% SDS, 50° C.

A vector (or plasmid) refers to discrete elements that can be used to introduce heterologous nucleic acid into cells for either expression or replication thereof. Vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art. An expression vector includes vectors capable of expressing DNA that is operatively linked with regulatory sequences, such as promoter regions, that are capable of effecting expression of such DNA fragments. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those that integrate into the host cell genome.

Disease prognosis refers to a forecast of the probable outcome of a disease or of a probable outcome resultant from a disease. Non-limiting examples of disease prognoses include likely relapse of disease, likely aggressiveness of disease, likely indolence of disease, likelihood of survival of the subject, likelihood of success in treating a disease, condition in which a particular treatment regimen is likely to be more effective than another treatment regimen, and combinations thereof.

Aggressiveness of a tumor or malignant cell is the capacity of one or more cells to attain a position in the body away from the tissue or organ of origin, attach to another portion of the body, and multiply. Experimentally, aggressiveness can be described in one or more manners, including, but not limited to, post-diagnosis survival of subject, relapse of tumor, and metastasis of tumor. Thus, in the disclosures provided herein, data indicative of time length of survival, relapse, non-relapse, time length for metastasis, or non-metastasis, are indicative of the aggressiveness of a tumor or a malignant cell. When survival is considered, one skilled in the art will recognize that aggressiveness is inversely related to the length of time of survival of the subject. When time length for metastasis is considered, one skilled in the art will recognize that aggressiveness is directly related to the length of time of survival of a subject. As used herein, indolence refers to non-aggressiveness of a tumor or malignant cell; thus, the more aggressive a tumor or cell, the less indolent, and vice versa. As an example of a cell attaining a position in the body away from the tissue or organ of origin, a malignant prostate cell can attain an extra-prostatic position, and thus have one characteristic of an aggressive malignant cell. Attachment of cells can be, for example, on the lymph node or bone marrow of a subject, or other sites known in the art.

A composition refers to any mixture. It can be a solution, a suspension, liquid, powder, a paste, aqueous, non-aqueous or any combination thereof.

A fluid is composition that can flow. Fluids thus encompass compositions that are in the form of semi-solids, pastes, solutions, aqueous mixtures, gels, lotions, creams and other such compositions.

### Cell-Type-Associated Patterns of Gene Expression

Primary tissues are composed of many (e.g., two or more) types of cells. Identification of genes expressed in a specific cell type present within a tissue in other methods can require physical separation of that cell type and the cell type's subsequent assay. Although it is possible to physically separate cells according to type, by methods such as laser capture microdissection, centrifugation, FACS, and the like, this is time consuming and costly and in certain embodiments impractical to perform. Known expression profiling assays (either RNA or protein) of primary tissues or other specimens containing multiple cell types either (1) do not take into account that multiple cell types are present or (2) physically separate the component cell types before performing the assay. Other analyses have been performed without regard to the presence of multiple cell types, thereby identifying markers indicative of a shift in the relative proportion of various cell types present in a sample, but not representative of a specific cell type. Previous analytic approaches cannot discern interactions between different types of cells.

Provided herein are methods, compositions and kits based on the development of a model, where the level of each gene product assayed can be correlated to a specific cell type. This approach for determination of cell-type-specific gene expression obviates the need for physical separation of cells from tissues or other specimens with heterogeneous cell content. Furthermore, this method permits determination of the interaction between the different types of cells contained in such heterogeneous mixtures, which would otherwise have been difficult or impossible had the cells been first physically separated and then assayed. Using the approaches provided herein, a number of biomarkers can be identified related to various diseases and disorders. Exemplified herein is the identification of biomarkers for prostate cancer and benign prostatic hypertophy. Such biomarkers can be used in diagnosis and prognosis and treatment decisions.

The methods, compositions, combinations and kits provided herein employ a regression-based approach for identification of cell-type-specific patterns of gene expression in samples containing more than one type of cell. In one example, the methods, compositions, combinations and kits provided herein employ a regression-based approach for identification of cell-type-specific patterns of gene expression in cancer. These methods, compositions, combinations and kits provided herein can be used in the identification of genes that are differentially expressed in malignant versus non-malignant cells and further identify tumor-dependent changes in gene expression of non-malignant cells associated with malignant cells relative to non-malignant cells not associated with malignant cells. The methods, compositions, combinations and kits provided herein also can be used in correlating a phenotype with gene expression in one or more cell types. For example such a method can include determining the relative content of each cell type in two or more related heterogeneous cell samples, wherein at least two of the samples do not contain the same relative content of each cell type, measuring overall levels of one or more gene expression analytes in each sample, determining the regression relationship between the relative content of each cell type and the measured overall levels, and calculating the level of each of the one or more analytes in each cell type according to the regression relationship, where gene expression levels correspond to the calculated levels of analytes. In another example such a method can include determining the relative content of each cell type in two or more related heterogeneous cell samples, wherein at least two of the samples do not contain the same relative content of each cell type, measuring overall levels of two or more gene expression analytes in each sample, determining the regression relationship between the relative content of each cell type and the measured overall levels, and calculating the level of each of the two or more analytes in each cell type according to the regression relationship, where gene expression levels correspond to the calculated levels of analytes. Such methods can further include identifying genes differentially expressed in at least one cell type relative to at least one other cell type. In such methods, the analyte can be a nucleic acid molecule and a protein.

The methods provided herein can be used for determining cell-type-specific gene expression in any heterogeneous cell population. The methods provided herein can find application in samples known to contain a variety of cell types, such as brain tissue samples and muscle tissue samples. The methods provided herein also can find application in samples in which separation of cell type can represent a tedious or time consuming operation, which is no longer required under the methods provided herein. Samples used in the present methods can be any of a variety of samples, including, but not limited to, blood, cells from blood (including, but not limited to, non-blood cells such as epithelial cells in blood), plasma, serum, spinal fluid, lymph fluid, skin, sputum, alimentary and genitourinary samples (including, but not limited to, urine, semen, seminal fluid, prostate aspirate, prostatic fluid, and fluid from the seminal vesicles), saliva, milk, tissue specimens (including, but not limited to, prostate tissue specimens), tumors, organs, and also samples of in vitro cell culture constituents.

In certain embodiments, the methods provided herein can be used to differentiate true markers of tumor cells, hyperplastic cells, and stromal cells of cancer. As exemplified herein, least squares regression using individual cell-type proportions can be used to produce clear predictions of cell-specific expression for a large number of genes. In an example provided herein applied to prostate cancer, many of these predictions are accepted on the basis of prior knowledge of prostate gene expression and biology, which provide confidence in the method. These are illustrated by numerous genes predicted to be preferentially expressed by stromal cells that are characteristic of connective tissue and only poorly expressed or absent in epithelial cells.

In some embodiments, the methods provided herein allow segregation of molecular tumor and nontumor markers into more discrete and informative groups. Thus, genes identified as tumor-associated can be further categorized into tumor versus stroma (epithelial versus mesenchymal) and tumor versus hyperplastic (perhaps reflecting true differences between the malignant cell and its hyperplastic counterpart). The methods provided herein can be used to distinguish tumor and non-tumor markers in a variety of cancers, including, without limitation, cancers classified by site such as cancer of the oral cavity and pharynx (lip, tongue, salivary gland, floor of mouth, gum and other mouth, nasopharynx, tonsil, oropharynx, hypopharynx, other oral/pharynx); cancers of the digestive system (esophagus; stomach; small intestine; colon and rectum; anus, anal canal, and anorectum; liver; intrahepatic bile duct; gallbladder; other biliary; pancreas; retroperitoneum; peritoneum, omentum, and mesentery; other digestive); cancers of the respiratory system (nasal cavity, middle ear, and sinuses; larynx; lung and bronchus; pleura; trachea, mediastinum, and other respiratory); cancers of the mesothelioma; bones and joints; and soft tissue, including heart; skin cancers, including melanomas and other non-epithelial skin cancers; Kaposi's sarcoma and breast cancer; cancer of the female genital system (cervix uteri; corpus uteri; uterus, nos; ovary; vagina; vulva; and other female genital); cancers of the male genital system (prostate gland; testis; penis; and other male genital); cancers of the urinary system (urinary bladder; kidney and renal pelvis; ureter; and other urinary); cancers of the eye and orbit; cancers of the brain and nervous system (brain; and other nervous system); cancers of the endocrine system (thyroid gland and other endocrine, including thymus); lymphomas (Hodgkin's disease and non-Hodgkin's lymphoma), multiple myeloma, and leukemias (lymphocytic leukemia; myeloid leukemia; monocytic leukemia; and other leukemias); and cancers classified by histological type, such as Neoplasm, malignant; carcinoma, NOS; carcinoma, undifferentiated, NOS; giant and spindle cell carcinoma; small cell carcinoma, NOS; papillary carcinoma, NOS; squamous cell carcinoma, NOS; lymphoepithelial carcinoma; basal cell carcinoma, NOS; pilomatrix carcinoma; transitional cell carcinoma, NOS; papillary transitional cell carcinoma; adenocarcinoma, NOS; gastrinoma, malignant; cholangiocarcinoma; hepatocellular carcinoma, NOS; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma, NOS; carcinoid tumor, malignant; bronchiolo-alveolar adenocarcinoma; papillary adenocarcinoma, NOS; ccarcinoma; acidophil carcinoma; oxyphilic adenocarcinoma; basophil carcinoma; clear cell adenocarcinoma, NOS; granular cell carcinoma; follicular adenocarcinoma, NOS; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma, NOS; papillary cystadenocarcinoma, NOS; papillary serous cystadenocarcinoma; mucinous cystadenocarcinoma, NOS; mucinous adenocarcinoma; signet ring cell carcinoma; infiltrating duct carcinoma; medullary carcinoma, NOS; lobular carcinoma; inflammatory carcinoma; Paget's disease, mammary; acinar cell carcinoma; adenosquamous carcinoma; adenocarcinoma with squamous metaplasia; thymoma, malignant; ovarian stromal tumor, malignant; thecoma, malignant; granulosa cell tumor, malignant; androblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma, NOS; amelanotic melanoma; superficial spreading melanoma; malignant melanoma in giant pigmented nevus; epithelioid cell melanoma; blue nevus, malignant; sarcoma, NOS; fibrosarcoma, NOS; fibrous histiocytoma, malignant; myxosarcoma; liposarcoma, NOS; leiomyosarcoma, NOS; rhabdomyosarcoma, NOS; embryonal rhabdomyosarcoma; alveolar rhabdomyosarcoma; stromal sarcoma, NOS; mixed tumor, malignant, NOS; Mullerian mixed tumor; nephroblastoma; hepatoblastoma; carcinosarcoma, NOS; mesenchymoma, malignant; Brenner tumor, malignant; phyllodes tumor, malignant; synovial sarcoma, NOS; mesothelioma, malignant; dysgerminoma; embryonal carcinoma, NOS; teratoma, malignant, NOS; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma, NOS; juxtacortical osteosarcoma; chondrosarcoma, NOS; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma, NOS; astrocytoma, NOS; protoplasmic astrocytoma; fibrillary astrocytoma; astroblastoma; glioblastoma, NOS; oligodendroglioma, NOS; oligodendroblastoma; primitive neuroectodermal; cerebellar sarcoma, NOS; ganglioneuroblastoma; neuroblastoma, NOS; retinoblastoma, NOS; olfactory neurogenic tumor; meningioma, malignant; neurofibrosarcoma; neurilemmoma, malignant; granular cell tumor, malignant; malignant lymphoma, NOS; Hodgkin's disease, NOS; Hodgkin's; paragranuloma, NOS; malignant lymphoma, small lymphocytic; malignant lymphoma, large cell, diffuse; malignant lymphoma, follicular, NOS; mycosis fungoides; other specified non-Hodgkin's lymphomas; malignant histiocytosis; multiple myeloma; mast cell sarcoma; immunoproliferative small intestinal disease; leukemia, NOS; lymphoid leukemia, NOS; plasma cell leukemia; erythroleukemia; lymphosarcoma cell leukemia; myeloid leukemia, NOS; basophilic leukemia; eosinophilic leukemia; monocytic leukemia, NOS; mast cell leukemia; megakaryoblastic leukemia; myeloid sarcoma; and hairy cell leukemia.

In an example comparing the results of a prostate tissue analysis using the methods provided herein to the results of previous methods, the vast majority of markers associated with normal prostate tissues in previous microarray-based studies relate to cells of the stroma. This result is not surprising given that normal samples can be composed of a relatively greater proportion of stromal cells.

In the example of prostate analysis, the strongest single discriminator between benign prostate hyperplasia (BPH) cells and tumor cells was CK15, a result confirmed by immunohistochemistry. CK15 has previously received little attention in this context, but BPH markers play an important role in the diagnosis of ambiguous clinical cases.

Transcripts whose expression levels have high covariance with cross-products of tissue proportions suggest that expression in one cell type depends on the proportion of another tissue, as would be expected in a paracrine mechanism. The stroma transcript with the highest dependence on tumor percentage was TGF-32. Another such stroma cell gene for which immunohistochemistry was practical was desmin, which showed altered staining in the tumor-associated stroma. In fact, a large number of typical stroma cell genes displayed dependence on the proportion of tumor, adding evidence to the speculation that tumor-associated stroma differs from non-associated stroma. Tumor-stroma paracrine signaling can be reflected in peritumor halos of altered gene expression that can present a much bigger target for detection than the tumor cells alone.

The methods provided herein provide a straightforward approach using simple and multiple linear regression to identify genes whose expression in tissue is specifically correlated with a specific cell type (e.g., in prostate tissue with either tumor cells, BPH epithelial cells or stromal cells). Context-dependent expression that is not readily attributable to single cell types is also recognized. The investigative approach described here is also applicable to a wide variety of tumor marker discovery investigations in a variety of tissues and organs. The exemplary prostate analysis results presented herein demonstrate the ability to identify a large number of gene candidates as specific products of various cells involved in prostate cancer pathogenesis.

A model for cell-specific gene expression is established by both (1) determination of the proportion of each constituent cell type (e.g., epithelium, stroma, tumor, or other discriminating entity) within a given type of tissue or specimen (e.g., prostate, breast, colon, marrow, and the like) and (2) assay of the expression profile (e.g., RNA or protein) of that same tissue or specimen. In some embodiments, cell type specific expression of a gene can be determined by fitting this model to data from a collection of tissue samples.

The methods provided herein can include a step of determining the relative content of each cell type in a heterogeneous sample. Identification of a cell type in a sample can include identifying cell types that are present in a sample in amounts greater than about 1%, 2%, 3%, 4% or 5% or greater than 1%, 2%, 3%, 4% or 5%.

Any of a variety of known methods for cell type identification can be used herein. For example, cell type can be determined by an individual skilled in the ability to identify cell types, such as a pathologist or a histologist. In another example, cell types can be determined by cell sorting and/or flow cytometry methods known in the art.

The methods provided herein can be used to determine that the nucleotide or proteins are differentially expressed in at least one cell type relative to at least one other cell type. Such genes include those that are up-regulated (i.e., expressed at a higher level), as well as those that are down-regulated (i.e., expressed at a lower level). Such genes also include sequences that have been altered (i.e., truncated sequences or sequences with substitutions, deletions or insertions, including point mutations) and show either the same expression profile or an altered profile. In certain embodiments, the genes can be from humans; however, as will be appreciated by those in the art, genes from other organisms can be useful in animal models of disease and drug evaluation; thus, other genes are provided, from vertebrates, including mammals, including rodents (e.g., rats, mice, hamsters, and guinea pigs), primates, and farm animals (e.g., sheep, goats, pigs, cows, and horses). In some cases, prokaryotic genes can be useful. Gene expression in any of a variety of organisms can be determined by methods provided herein or otherwise known in the art.

Gene products measured according to the methods provided herein can be nucleic acid molecules, including, but not limited to mRNA or an amplicate or complement thereof, polypeptides, or fragments thereof. Methods and compositions for the detection of nucleic acid molecules and proteins are known in the art. For example, oligonucleotide probes and primers can be used in the detection of nucleic acid molecules, and antibodies can be used in the detection of polypeptides.

In the methods provided herein, one or more gene products can be detected. In some embodiments, two or more gene products are detected. In other embodiments, 3 or more, 4 or more, 5 or more, 7 or more, 10 or more 15 or more, 20 or more 25, or more, 35 or more, 50 or more, 75 or more, or 100 or more gene products can be detected in the methods provided herein.

The expression levels of the marker genes in a sample can be determined by any method or composition known in the art. The expression level can be determined by isolating and determining the level (i.e., amount) of nucleic acid transcribed from each marker gene. Alternatively, or additionally, the level of specific proteins translated from mRNA transcribed from a marker gene can be determined.

Determining the level of expression of specific marker genes can be accomplished by determining the amount of mRNA, or polynucleotides derived therefrom, or protein present in a sample. Any method for determining protein or RNA levels can be used. For example, protein or RNA is isolated from a sample and separated by gel electrophoresis. The separated protein or RNA is then transferred to a solid support, such as a filter. Nucleic acid or protein (e.g., antibody) probes representing one or more markers are then hybridized to the filter by hybridization, and the amount of marker-derived protein or RNA is determined. Such determination can be visual, or machine-aided, for example, by use of a densitometer. Another method of determining protein or RNA levels is by use of a dot-blot or a slot-blot. In this method, protein, RNA, or nucleic acid derived therefrom, from a sample is labeled. The protein, RNA or nucleic acid derived therefrom is then hybridized to a filter containing oligonucleotides or antibodies derived from one or more marker genes, wherein the oligonucleotides or antibodies are placed upon the filter at discrete, easily-identifiable locations. Binding, or lack thereof, of the labeled protein or RNA to the filter is determined visually or by densitometer. Proteins or polynucleotides can be labeled using a radiolabel or a fluorescent (i.e., visible) label.

Methods provided herein can be used to detect mRNA or amplicates thereof, and any fragment thereof. In one example, introns of mRNA or amplicate or fragment thereof can be detected. Processing of mRNA can include splicing, in which introns are removed from the transcript. Detection of introns can be used to detect the presence of the entire mRNA, and also can be used to detect processing of the mRNA, for example, when the intron region alone (e.g., intron not attached to any exons) is detected.

In another embodiment, methods provided herein can be used to detect polypeptides and modifications thereof, where a modification of a polypeptide can be a post-translation modification such as lipidylation, glycosylation, activating proteolysis, and others known in the art, or can include degradational modification such as proteolytic fragments and ubiquitinated polypeptides.

These examples are not intended to be limiting; other methods of determining protein or RNA abundance are known in the art.

Alternatively, proteins can be separated by two-dimensional gel electrophoresis systems. Two-dimensional gel electrophoresis is well-known in the art and can involve isoelectric focusing along a first dimension followed by SDS-PAGE electrophoresis along a second dimension. See, e.g., Hames et al. (1990) Gel Electrophoresis of Proteins: A Practical Approach, IRL Press, New York; Shevchenko et al. (1996) Proc. Natl. Acad. Sci. USA 93:1440-1445; Sagliocco et al. (1996) Yeast 12:1519-1533; and Lander (1996) Science 274:536-539. The resulting electropherograms can be analyzed by numerous techniques, including mass spectrometric techniques, western blotting and immunoblot analysis using polyclonal and monoclonal antibodies.

Alternatively, marker-derived protein levels can be determined by constructing an antibody microarray in which binding sites comprise immobilized antibodies, such as monoclonal antibodies, specific to a plurality of protein species encoded by the cell genome. Antibodies can be present for a substantial fraction of the marker-derived proteins of interest. Methods for making monoclonal antibodies are well known (see, e.g., Harlow and Lane (1988) Antibodies: A Laboratory Manual, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array, and their binding is assayed with assays known in the art. The expression, and the level of expression, of proteins of diagnostic or prognostic interest can be detected through immunohistochemical staining of tissue slices or sections.

In another embodiment, expression of marker genes in a number of tissue specimens can be characterized using a tissue array (Kononen et al. (1998) Nat. Med. 4:844-847). In a tissue array, multiple tissue samples are assessed on the same microarray. The arrays allow in situ detection of RNA and protein levels; consecutive sections allow the analysis of multiple samples simultaneously.

In some embodiments, polynucleotide microarrays are used to measure expression so that the expression status of each of the markers above is assessed simultaneously. In one embodiment, the microarrays provided herein are oligonucleotide or cDNA arrays comprising probes hybridizable to the genes corresponding to the marker genes described herein. A microarray as provided herein can comprise probes hybridizable to the genes corresponding to markers able to distinguish cells, identify phenotypes, identify a disease or disorder, or provide a prognosis of a disease or disorder (e.g., a classifier as described herein). For example, provided herein are polynucleotide arrays comprising probes to a subset or subsets of at least 2, 5, 10, 15, 20, 30, 40, 50, 75, 100, or more than 100 genetic markers, up to the full set of markers present in a classifier as described in the Examples below. Also provided herein are probes to markers with a modified t statistic greater than or equal to 2.5, 3, 3.5, 4, 4.5 or 5. Also provided herein are probes to markers with a modified t statistic less than or equal to −2.5, −3, −3.5, −4, −4.5 or −5. In specific embodiments, the invention provides combinations such as arrays in which the markers described herein comprise at least 50%, 60%, 70%, 80%, 85%, 90%, 95% or 98% of the probes on the combination or array.

General methods pertaining to the construction of microarrays comprising the marker sets and/or subsets above are known in the art as described herein.

Microarrays can be prepared by selecting probes that comprise a polypeptide or polynucleotide sequence, and then immobilizing such probes to a solid support or surface.

For example, the probes can comprise DNA sequences, RNA sequences, or antibodies. The probes can also comprise amino acid, DNA and/or RNA analogues, or combinations thereof. The probes can be prepared by any method known in the art.

The probe or probes used in the methods of the invention can be immobilized to a solid support which can be either porous or non-porous. For example, the probes of the can be attached to a nitrocellulose or nylon membrane or filter. Alternatively, the solid support or surface can be a glass or plastic surface. In another embodiment, hybridization levels are measured to microarrays of probes consisting of a solid phase on the surface of which are immobilized a population of probes. The solid phase can be a nonporous or, optionally, a porous material such as a gel.

In another embodiment, the microarrays are addressable arrays, such as positionally addressable arrays. More specifically, each probe of the array can be located at a known, predetermined position on the solid support such that the identity (i.e., the sequence) of each probe can be determined from its position in the array (i.e., on the support or surface).

A skilled artisan will appreciate that positive control probes, e.g., probes known to be complementary and hybridizable to sequences in target polynucleotide molecules, and negative control probes, e.g., probes known to not be complementary and hybridizable to sequences in target polynucleotide molecules, can be included on the array. In one embodiment, positive controls can be synthesized along the perimeter of the array. In another embodiment, positive controls can be synthesized in diagonal stripes across the array. Other variations are known in the art. Probes can be immobilized on the to solid surface by any of a variety of methods known in the art.

In certain embodiments, this model can be further extended to include sample characteristics, such as cell or organism phenotypes, allowing cell type specific expression to be linked to observable indicia such as clinical indicators and prognosis (e.g., clinical disease progression, response to therapy, and the like). In one embodiment, a model for prostate tissue is provided, resulting in identification of cell-type-specific markers of cancer, epithelial hypertrophy, and disease progression. In another embodiment, a method for studying differential gene expression between subjects with cancers that relapse and those with cancers that do not relapse, is disclosed. Also provided is the framework for studying mixed cell type samples and more flexible models allowing for cross-talk among genes in a sample. Also provided are extensions to defining differences in expression between samples with different characteristics, such as samples from subjects who subsequently relapse versus those who do not.

### Statistical Treatment

The methods provided herein include determining the regression relationship between relative cell content and measured expression levels. For example, the regression relationship can be determined by determining the regression of measured expression levels on cell proportions. Statistical methods for determining regression relationships between variables are known in the art. Such general statistical methods can be used in accordance with the teachings provided herein regarding regression of measured expression levels on cell proportions.

The methods provided herein also include calculating the level of analytes in each cell type based on the regression relationship between relative cell content and expression levels. The regression relationship can be determined according to methods provided herein, and, based on the regression relationship, the level of a particular analyte can be calculated for a particular cell type. The methods provided herein can permit the calculation of any of a variety of analyte for particular cell types. For example, the methods provided herein can permit calculation of a single analyte for a single cell type, or can permit calculation of a plurality of analytes for a single cell type, or can permit calculation of a single analyte for a plurality of cell types, or can permit calculation of a plurality of analytes for a plurality of cell types. Thus, the number of analytes whose level can be calculated for a particular cell type can range from a single analyte to the total number of analytes measured (e.g., the total number of analytes measured using a microarray). In another embodiment, the total number of cell types for which analyte levels can be calculated can range from a single cell type, to all cell types present in a sample at sufficient levels. The levels of analyte for a particular cell type can be used to estimate expression levels of the corresponding gene, as provided elsewhere herein.

The methods provided herein also can include identifying genes differentially expressed in a first cell type relative to a second cell type. Expression levels of one or more genes in a particular cell type can be compared to one or more additional cell types. Differences in expression levels can be represented in any of a variety of manners known in the art, including mathematical or statistical representations, as provided herein. For example, differences in expression level can be represented as a modified t statistic, as described elsewhere herein.

The methods provided herein also can serve as the basis for methods of indicating the presence of a particular cell type in a subject. The methods provided herein can be used for identifying the expression levels in particular cell types. Using any of a variety of classifier methods known in the art, such as a naïve Bayes classifier, gene expression levels in cells of a sample from a subject can be compared to reference expression levels to determine the presence of absence, and, optionally, the relative amount, of a particular cell type in the sample. For example, the markers provided herein as associated with prostate tumor, stroma or BPH can be selected in a prostate tumor classifier in accordance with the modified t statistic associated with each marker provided in the Tables herein. Methods for using a modified t statistic in classifier methods are provided herein and also are known in the art. In another embodiment, the methods provided herein can be used in phenotype-indicating methods such as diagnostic or prognostic methods, in which the gene expression levels in a sample from a subject can be compared to references indicative of one or more particular phenotypes.

For purposes of exemplification, and not for purposes of limitation, an exemplary method of determining gene expression levels in one or more cell types in a heterogeneous cell sample is provided as follows. Suppose that there are four cell types: BPH, Tumor, Stroma, fᵢⱼ(y), iε{BPH, Tumor, Stroma, Cystic Atrophy} and Cystic Atrophy. Supposing that each cell type has a (possibly) different distribution for y, the expression level for a gene j, denoted by:

and that sample k has proportions

Xₖ=(x_{k,BPH},x_{k,Tumor},x_{k,Stroma},x_{k,Cystic Atrophy})

of each cell type is studied. The distribution of the expression level for gene j is then

$${g}_{j} ( y \vert {X}_{k} ) = {\sum }_{i} {x}_{ki} {f}_{ij} ( y )$$

if the expression levels are additive in the cell proportions as they would be if each cell's expression level depends only on the type of cell (and not, say, on what other types of cells can be present in the sample). In a later section this formulation is extended to cases in which the expression of a given cell type depends on what other types of cells are present.

The average expression level in a sample is then the weighted average of the expectations with weights corresponding to the cell proportions:

$${E}_{{g}_{j}} ( y \vert {X}_{k} ) = {\sum }_{i} {x}_{ki} {E}_{{f}_{ij}} ( y )$$

$$or$$

$${y}_{jk} = {\sum }_{i} {x}_{ki} {β}_{ij} + {ε}_{jk}$$

$$where$$

$${E}_{{f}_{ij}} ( y ) = {β}_{ij} and {ε}_{jk} = {y}_{jk} - {E}_{{g}_{j}} ( y \vert {X}_{k} )$$

This is the known form for a multiple linear regression equation (without specifying an intercept), and when multiple samples are available one can estimate the βᵢⱼ. Once these estimates are in hand, estimates for the differences in gene expression of two cell types are of the form:

\hat{β}ᵢ_{₁ⱼ}−\hat{β}ᵢ_{₂ⱼ}

and standard methods for testing linear hypotheses about the coefficients βᵢⱼ can be applied to test whether the average expression levels of cell types i₁ and i₂ are different. The term ‘expression levels’ as used in this exemplification of the method is used in a generic sense: ‘expression levels’ could be readings of mRNA levels, cRNA levels, protein levels, fluorescent intensity from a feature on an array, the logarithm of that reading, some highly post-processed reading, and the like. Thus, differences in the coefficients can correspond to differences, log ratios, or some other functions of the underlying transcript abundance.

For computational convenience, one may in certain embodiments use Z=XT and γ=T⁻¹β setting up T so that one column of T has all zeroes but for a one in position i₁ and a minus one in position i₂ such as

$$T = ( \begin{matrix}1 & 1 & - 1 & 0 \\ 1 & 1 & 1 & 0 \\ 1 & 0 & 0 & 1 \\ 1 & 0 & 0 & 0\end{matrix} )$$

The columns of Z that result are the unit vector (all ones), x_{k,BPH}+x_{k,Tumor}, x_{k,BPH}, −x_{k,Tumor}, and x_{k,Stroma}. With this setup, twice the coefficient of x_{k,BPH}−x_{k,Tumor} estimates the average difference in expression level of a tumor cell versus a BPH cell. With this parametrization, standard software can be used to provide an estimate and a tesmodified t statistic for the average difference of tumor and BPH cells. Further, this can simplify the specification of restricted models in which two or more of the tissue components have the same average expression level.

The data for a study can contain a large number of samples from a smaller number of different men. It is plausible that the samples from one man may tend to share a common level of expression for a given gene, differences among his cells according to their type notwithstanding. This will tend to lead to positive covariance among the measurements of expression level within men. Ordinary least squares (OLS) estimates are less than fully efficient in such circumstances. One alternative to OLS is to use a weighted least squares approach that treats a collection of samples from a single subject as having a common (non-negative) covariance and identical variances.

The estimating equation for this setup can be solved via iterative methods using software such as the gee library from R (Ihaka and Gentleman (1996) J. Comp. Graph. Stat. 5:299-314). When the estimated covariance is negative—as sometimes happens when there is an extreme outlier in the dataset—it can be fixed at zero. Also the sandwich estimate (Liang and Zeger (1986) Biometrika 73:13-22) of the covariance structure can be used.

The estimating equation approach will provide a tesmodified t statistic for a single transcript. Assessment of differential expression among a group of 12625 transcripts is handled by permutation methods that honor a suitable null model. That null model is obtained by regressing the expression level on all design terms except for the ‘BPH-tumor’ term using the exchangeable, non-negative correlation structure just mentioned. For performing permutation tests, the correlation structure in the residuals can be accounted for. Let κ₁ be the set of n₁ indexes of samples for subject 1. First, we find yⱼₖ−ŷⱼₖ=eₗₖ, kεκ₁, as the residuals from that fitted null model for subject 1. The inverse square root of the correlation matrix of these residuals is used to transform them, i.e., \tilde{e}ⱼ=σ^{-1/2}eⱼ, where σ is the (block diagonal) correlation matrix obtained by substituting the estimate of r from gee as the off-diagonal elements of blocks corresponding to measurements for each subject and e_{j.} and \tilde{e}ⱼ are the vector of residuals and transformed residuals for all subjects for gene j. Asymptotically, the \tilde{e}ⱼₖ have means and covariances equal to zero. Random permutations of these, \tilde{e}ⱼ⁽ⁱ⁾, i=M, are obtained and used to form pseudo-observations:

\tilde{y}_{j.}⁽ⁱ⁾=\tilde{y}_{j.}+σ^{1/2}\tilde{e}_{j.}⁽ⁱ⁾

This permutation scheme preserves the null model and enforces its correlation structure asymptotically.

In certain embodiments, the contribution of each type of cell does not depend on what other cell types are present in the sample. However, there can be instances in which contribution of each type of cell does depend on other cell types present in the sample. It may happen that putatively ‘normal’ cells exhibit genomic features that influence both their expression profiles and their potential to become malignant. Such cells would exhibit the same expression pattern when located in normal tissue, but are more likely to be found in samples that also have tumor cells in them. Another possible effect is that signals generated by tumor cells trigger expression changes in nearby cells that would not be seen if those same cells were located in wholly normal tissue. In either case, the contribution of a cell may be more or less than in another tissue environment leading to a setup in which the contributions of individual cell types to the overall profile depend on the proportions of all types present, viz.

$${g}_{j} ( y \vert {X}_{k} ) = {\sum }_{i} {x}_{ki} {f}_{ij} ( y \vert {X}_{k} )$$

as do the expected proportions

$${E}_{{g}_{j}} ( y \vert {X}_{k} ) = {\sum }_{i} {x}_{ki} {E}_{{f}_{ij}} ( y \vert {X}_{k} )$$

$$or$$

$${y}_{jk} = {\sum }_{i} {x}_{ki} {β}_{ij} ( {X}_{k} ) + {ε}_{jk}$$

The methods used herein above can still be applied in the context provided some calculable form is given for βᵢⱼ(Xₖ). One choice is given by

βᵢⱼ(Xₖ)=(φⱼR(Xₖ))ᵢ

where Φⱼ is a 4×m matrix of unknown coefficients and R(Xₖ) is a column vector of m elements. This reduces to the case in which each cell's expression level depends only on the type of cell when Φⱼ is 4×1 matrix and R(Xₖ) is just ‘1’.

Consider the case:

$$\begin{matrix}{φ}_{j} ( {X}_{k} ) R ( {X}_{k} ) = ( \begin{matrix}{v}_{{B}_{j}} & {v}_{{B}_{j}} & {v}_{{B}_{j}} & {v}_{{B}_{j}} \\ {v}_{{T}_{j}} & {v}_{{T}_{j}} & {v}_{{T}_{j}} & {v}_{{T}_{j}} \\ {v}_{{S}_{j}} & {v}_{{S}_{j}} + {δ}_{j} & {v}_{{S}_{j}} & {v}_{{S}_{j}} \\ {v}_{{C}_{j}} & {v}_{{C}_{j}} & {v}_{{C}_{j}} & {v}_{{C}_{j}}\end{matrix} ) ( \begin{matrix}{x}_{k , B} \\ {x}_{k , T} \\ {x}_{k , S} \\ {x}_{k , C}\end{matrix} ) \\ = ( \begin{matrix}{v}_{{B}_{j}} \\ {v}_{{T}_{j}} \\ {v}_{{S}_{j}} + {δ}_{j} {x}_{k , T} \\ {v}_{{C}_{j}}\end{matrix} )\end{matrix}$$

$$\begin{matrix}{φ}_{j} ( {X}_{k} ) R ( {X}_{k} ) = ( \begin{matrix}{v}_{{B}_{j}} & {v}_{{B}_{j}} & {v}_{{B}_{j}} & {v}_{{B}_{j}} \\ {v}_{{T}_{j}} & {v}_{{T}_{j}} & {v}_{{T}_{j}} & {v}_{{T}_{j}} \\ {v}_{{S}_{j}} & {v}_{{S}_{j}} + {δ}_{j} & {v}_{{S}_{j}} & {v}_{{S}_{j}} \\ {v}_{{C}_{j}} & {v}_{{C}_{j}} & {v}_{{C}_{j}} & {v}_{{C}_{j}}\end{matrix} ) ( \begin{matrix}{x}_{k , B} \\ {x}_{k , T} \\ {x}_{k , S} \\ {x}_{k , C}\end{matrix} ) \\ = ( \begin{matrix}{v}_{{B}_{j}} \\ {v}_{{T}_{j}} \\ {v}_{{S}_{j}} + {δ}_{j} {x}_{k , T} \\ {v}_{{C}_{j}}\end{matrix} )\end{matrix}$$

(and recall that ΣⱼX_{k,j}=1.) Here the subscript for Tumor has been abbreviated T₁ etc., for brevity. This setup provides that BPH (B), tumor, and cystic atrophy (C) cells have expression profiles that do not depend on the other cell types in the sample. However, the expression levels of stromal cells (S) depend on the proportion of tumor cells as reflected by the coefficient δⱼ. Notice that

is linear in X_{k,B}, X_{k,T}, X_{k,S}, X_{k,C}, and X_{k,S}X_{k,T} with the unknown coefficients being

(XₖφⱼR(Xₖ)=x_{k,B}ν_{Bj}+x_{k,T}ν_{Tj}+x_{k,S}ν_{Sj}+x_{k,S}x_{k,T}δⱼ+x_{k,C}84 _{Cj}multipliers of those terms. So, the unknowns in this case are linear functions of the gene expression levels and can be determined using standard linear models as was done earlier. The only change here is the addition of the product of X_{k,S} and X_{k,T}. Such a product, when significant, is termed an “interaction” and refers to the product archiving a significance level owing to a correlation of X_{k,S} with X_{k,T}. Thus, it is possible to accommodate variations in gene expression that occur when the level of a transcript in one cell type is influenced by the amount of another cell type in the sample. In one aspect, a setup involving a dependency of tumor on the amount of stroma

$$\begin{matrix}{φ}_{j} ( {X}_{k} ) R ( {X}_{k} ) = ( \begin{matrix}{v}_{{B}_{j}} & {v}_{{B}_{j}} & {v}_{{B}_{j}} & {v}_{{B}_{j}} \\ {v}_{{T}_{j}} & {v}_{{T}_{j}} & {v}_{{T}_{j}} + {δ}_{j} & {v}_{{T}_{j}} \\ {v}_{{S}_{j}} & {v}_{{S}_{j}} & {v}_{{S}_{j}} & {v}_{{S}_{j}} \\ {v}_{{C}_{j}} & {v}_{{C}_{j}} & {v}_{{C}_{j}} & {v}_{{C}_{j}}\end{matrix} ) ( \begin{matrix}{x}_{k , B} \\ {x}_{k , T} \\ {x}_{k , S} \\ {x}_{k , C}\end{matrix} ) \\ = ( \begin{matrix}{v}_{{B}_{j}} \\ {v}_{{T}_{j}} + {δ}_{j} {x}_{k , T} \\ {v}_{{S}_{j}} \\ {v}_{{C}_{j}}\end{matrix} )\end{matrix}$$

the expression for XₖΦⱼR(Xₖ) is precisely as it was just above.

Accordingly, one can screen for dependencies by including as regressors products of the proportions of cell types. In certain embodiments, it may not be possible to detect interactions if two different cell types experience equal and opposite changes—one type expressing more with increases in the other and the other expressing less with increases in the first. In one embodiment, dependence of gene expression refers to the dependence of gene expression in one cell type on the level of gene expression in another cell type. In another embodiment, dependence of gene expression refers to the dependence of gene expression in one cell type on the amount of another cell type.

The contribution of each type of cell can depend on what other cell types are present in the sample, but also can depend on other characteristics of the sample, such as clinical characteristics of the subject who contributed it. For example, clinical characteristics such as disease symptoms, disease prognosis such as relapse and/or aggressiveness of disease, likelihood of success in treating a disease, likelihood of survival, condition in which a particular treatment regimen is likely to be more effective than another treatment regimen, can be correlated with cell expression. For example, cell type specific gene expression can differ between a subject with a cancer that does not relapse after treatment and a subject with a cancer that does relapse after treatment. In this case, the contribution of a cell type may be more or less than in another subject leading to an instance in which the contributions of individual cell types to the overall profile depend on the characteristics of the subject or sample. Here, the model used earlier is extended to allow for dependence on a vector of sample specific covariates, Zₖ:

$${g}_{j} ( y \vert {X}_{k} , {Z}_{k} ) = {\sum }_{i}^{ } {x}_{ki} {f}_{ij} ( y \vert {X}_{k} , {Z}_{k} )$$

as do the expected proportions:

$${E}_{gj} ( y \vert {X}_{k} , {Z}_{k} ) = {\sum }_{i}^{ } {x}_{ki} {E}_{{f}_{ij}} ( y \vert {X}_{k} , {Z}_{k} )$$

$$or$$

$${y}_{jk} = {\sum }_{i}^{ } {x}_{ki} {β}_{ij} ( {X}_{k} , {Z}_{k} ) + {∈}_{jk} \\ where$$

$${E}_{{f}_{ij}} ( y \vert {X}_{k} , {Z}_{k} ) = {β}_{ij} ( {X}_{k} , {Z}_{k} )$$

$$and \\ {∈}_{jk} = {y}_{jk} - {E}_{gj} ( y \vert {X}_{k} , {Z}_{k} ) .$$

The methods used herein above can still be applied in this context provided some reasonable form is given for βᵢⱼ(Xₖ,Zₖ). One useful choice is given by:

βᵢⱼ(Xₖ,Zₖ)=(φⱼR(Zₖ))ᵢ

Where Φⱼ is a 4×m matrix of unknown coefficients and R(Zₖ) is a column vector of m elements.

Consider how this would be used to study differences in gene expression among subjects who relapse and those who do not. In this case, Zₖ is an indicator variable taking the value zero for samples of subjects who do not relapse and one for those who do. Then

$$R ( {Z}_{k} ) = ( \begin{matrix}1 \\ {Z}_{k}\end{matrix} )$$

and Φⱼ is a four by two matrix of coefficients:

$${φ}_{j} = ( \begin{matrix}{v}_{Bj} & {δ}_{Bj} \\ {v}_{Tj} & {δ}_{Tj} \\ {v}_{Sj} & {δ}_{Sj} \\ {v}_{Cj} & {δ}_{Cj}\end{matrix} )$$

Notice that this leads to

XₖφⱼR(Zₖ)=(x_{k,B}ν_{Bj}+x_{k,T}ν_{Tj}+x_{k,S}ν_{Sj}+x_{k,C}ν_{Cj}+x_{k,B}Zₖδ_{Bj}+x_{k,T}Zₖδ_{Tj}+x_{k,S}Zₖδ_{Sj}+x_{k,C}Zₖδ_{Cj}

The ν coefficients give the average expression of the different cell types in subjects who do not relapse, while the δ coefficients give the difference between the average expression of the different cell types in subjects who do relapse and those who do not. Thus, a non-zero value of δ_{T} would indicate that in tumor cells, the average expression level differs for subjects who relapse and those who do not. The above equation is linear in its coefficients, so standard statistical methods can be applied to estimation and inference on the coefficients. Extensions that allow β to depend on both cell proportions and on sample covariates can be determined according to the teachings provided herein or other methods known in the art.

### Nucleic Acids

Provided herein are tables and exhibits listing probe sets and genes associated with the probe set, including, for some tables, GENBANK accession number, and/or locus ID. The tables may include modified t statistics for an Affymetrix microarrays, including associated t statistics for BPH, tumor, stroma and cystic atrophy, for example. Probe IDs for the microarray that map to Probe IDs for a different microarray, and the mapping itself, also may be provided, where the mapping can represent. Probe IDs of microarrays that can hybridize to the same gene. By virtue of such mapping, Probe IDs can be associated with nucleotide sequences. Tables also may list the top genes identified as up- and down-regulated in prostate tumor cells of relapse patients, calculated by linear regression including all samples with prostate cancer. Genes that have greater than, for example, a 1.5 fold ratio of predicted expression between relapse and non-relapse tissue can be identified, as can an absolute difference in expression that exceeds the expression level reported for most genes queried by the array.

The tables provided herein also may list the top genes identified as up- and down-regulated in tumors and/or prostate stroma of relapse patients, calculated by linear regression including all samples with prostate cancer. Exemplary genes whose expression can be examined in methods for identifying or characterizing a sample may be provided, as well as Probe IDs that can be used for such gene expression identification.

Splice variants of genes also may be useful for determining diagnosis and prognosis of prostate cancer. As will be understood in the art, multiple splicing combinations are provided for some genes. Reference herein to one or more genes (including reference to products of genes) also contemplates reference to spliced gene sequences. Similarly, reference herein to one or more protein gene products also contemplates proteins translated from splice variants.

Exemplary, non-limiting examples of genes whose products can be detected in the methods provided herein include IGF-1, microsimino protein, and MTA-1. In one embodiment detection of the expression of one or more of these genes can be performed in combination with detection of expression of one or more additional genes as listed in the tables herein.

Uses of probes and detection of genes identified in the tables may be described and exemplified herein. It is contemplated herein that uses and methods similar to those exemplified can be applied to the probe and gene nucleotide sequences in accordance with the teachings provided herein.

The isolated nucleic acids can contain least 10 nucleotides, 25 nucleotides, 50 nucleotides, 100 nucleotides, 150 nucleotides, or 200 nucleotides or more, contiguous nucleotides of a gene listed herein. In another embodiment, the nucleic acids are smaller than 35, 200 or 500 nucleotides in length.

Also provided are fragments of the above nucleic acids that can be used as probes or primers and that contain at least about 10 nucleotides, at least about 14 nucleotides, at least about 16 nucleotides, or at least about 30 nucleotides. The length of the probe or primer is a function of the size of the genome probed; the larger the genome, the longer the probe or primer required for specific hybridization to a single site. Those of skill in the art can select appropriately sized probes and primers. Probes and primers as described can be single-stranded. Double stranded probes and primers also can be used, if they are denatured when used. Probes and primers derived from the nucleic acid molecules are provided. Such probes and primers contain at least 8, 14, 16, 30, 100 or more contiguous nucleotides. The probes and primers are optionally labeled with a detectable label, such as a radiolabel or a fluorescent tag, or can be mass differentiated for detection by mass spectrometry or other means. Also provided is an isolated nucleic acid molecule that includes the sequence of molecules that is complementary to a nucleotide. Double-stranded RNA (dsRNA), such as RNAi is also provided.

Plasmids and vectors containing the nucleic acid molecules are also provided. Cells containing the vectors, including cells that express the encoded proteins are provided. The cell can be a bacterial cell, a yeast cell, a fungal cell, a plant cell, an insect cell or an animal cell.

For recombinant expression of one or more genes, the nucleic acid containing all or a portion of the nucleotide sequence encoding the genes can be inserted into an appropriate expression vector, i.e., a vector that contains the elements for the transcription and translation of the inserted protein coding sequence. Transcriptional and translational signals also can be supplied by the native promoter for the genes, and/or their flanking regions.

Also provided are vectors that contain nucleic acid encoding a gene listed herein. Cells containing the vectors are also provided. The cells include eukaryotic and prokaryotic cells, and the vectors are any suitable for use therein.

Prokaryotic and eukaryotic cells containing the vectors are provided. Such cells include bacterial cells, yeast cells, fungal cells, plant cells, insect cells and animal cells. The cells can be used to produce an oligonucleotide or polypeptide gene products by (a) growing the above-described cells under conditions whereby the encoded gene is expressed by the cell, and then (b) recovering the expressed compound.

A variety of host-vector systems can be used to express the protein coding sequence. These include, but are not limited to, mammalian cell systems infected with virus (e.g., vaccinia virus and adenovirus); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system used, any one of a number of suitable transcription and translation elements can be used.

Any methods known to those of skill in the art for the insertion of nucleic acid fragments into a vector can be used to construct expression vectors containing a chimeric gene containing appropriate transcriptional/translational control signals and protein coding sequences. These methods can include in vitro recombinant DNA and synthetic techniques and in vivo recombinants (genetic recombination). Expression of nucleic acid sequences encoding polypeptide can be regulated by a second nucleic acid sequence so that the genes or fragments thereof are expressed in a host transformed with the recombinant DNA molecule(s). For example, expression of the proteins can be controlled by any promoter/enhancer known in the art.

### Proteins

Protein products of the genes listed herein, derivatives, and analogs can be produced by various methods known in the art. For example, once a recombinant cell expressing such a polypeptide, or a domain, fragment or derivative thereof, is identified, the individual gene product can be isolated and analyzed. This is achieved by assays based on the physical and/or functional properties of the protein, including, but not limited to, radioactive labeling of the product followed by analysis by gel electrophoresis, immunoassay, cross-linking to marker-labeled product, and assays of protein activity or antibody binding.

Polypeptides can be isolated and purified by standard methods known in the art (either from natural sources or recombinant host cells expressing the complexes or proteins), including but not restricted to column chromatography (e.g., ion exchange, affinity, gel exclusion, reversed-phase high pressure and fast protein liquid), differential centrifugation, differential solubility, or by any other standard technique used for the purification of proteins. Functional properties can be evaluated using any suitable assay known in the art.

Manipulations of polypeptide sequences can be made at the protein level. Also contemplated herein are polypeptide proteins, domains thereof, derivatives or analogs or fragments thereof, which are differentially modified during or after translation, e.g., by glycosylation, acetylation, phosphorylation, amidation, derivatization by known protecting/blocking groups, proteolytic cleavage, linkage to an antibody molecule or other cellular ligand. Any of numerous chemical modifications can be carried out by known techniques, including but not limited to specific chemical cleavage by cyanogen bromide, trypsin, chymotrypsin, papain, V8 protease, NaBH4, acetylation, formulation, oxidation, reduction, metabolic synthesis in the presence of tunicamycin and other such agents.

In addition, domains, analogs and derivatives of a polypeptide provided herein can be chemically synthesized. For example, a peptide corresponding to a portion of a polypeptide provided herein, which includes the desired domain or which mediates the desired activity in vitro can be synthesized by use of a peptide synthesizer. Furthermore, if desired, nonclassical amino acids or chemical amino acid analogs can be introduced as a substitution or addition into the polypeptide sequence. Non-classical amino acids include but are not limited to the D-isomers of the common amino acids, a-amino isobutyric acid, 4-aminobutyric acid, Abu, 2-aminobutyric acid, .epsilon.-Abu, e-Ahx, 6-amino hexanoic acid, Aib, 2-amino isobutyric acid, 3-amino propionoic acid, ornithine, norleucine, norvaline, hydroxyproline, sarcosine, citrulline, cysteic acid, t-butylglycine, t-butylalanine, phenylglycine, cyclohexylalanine, .beta.-alanine, fluoro-amino acids, designer amino acids such as .beta.-methyl amino acids, Ca-methyl amino acids, Na-methyl amino acids, and amino acid analogs in general. Furthermore, the amino acid can be D (dextrorotary) or L (levorotary).

### Screening Methods

Oligonucleotide or polypeptide gene products can be used in a variety of methods to identify compounds that modulate the activity thereof. Nucleotide sequences and genes can be identified in different cell types and in the same cell type in which subject have different phenotypes. Methods are provided herein for screening compounds can include contacting cells with a compound and measuring gene expression levels, wherein a change in expression levels relative to a reference identifies the compound as a compound that modulates a gene expression.

Also provided herein are methods for identification and isolation of agents, such as compounds that bind to products of the genes listed herein. The assays are designed to identify agents that bind to the RNA or polypeptide gene product. The identified compounds are candidates or leads for identification of compounds for treatments of tumors and other disorders and diseases.

A variety of methods can be used, as known in the art. These methods can be performed in solution or in solid phase reactions.

Methods for identifying an agent, such as a compound, that specifically binds to an oligonucleotide or polypeptide encoded by a gene as listed herein also are provided. The method can be practiced by (a) contacting the gene product with one or a plurality of test agents under conditions conducive to binding between the gene product and an agent; and (b) identifying one or more agents within the one or plurality that specifically binds to the gene product. Compounds or agents to be identified can originate from biological samples or from libraries, including, but are not limited to, combinatorial libraries. Exemplary libraries can be fusion-protein-displayed peptide libraries in which random peptides or proteins are presented on the surface of phage particles or proteins expressed from plasmids; support-bound synthetic chemical libraries in which individual compounds or mixtures of compounds are presented on insoluble matrices, such as resin beads, or other libraries known in the art.

### Modulators of the Activity of Gene Products

Provided herein are compounds that modulate the activity of a gene product. These compounds can act by directly interacting with the polypeptide or by altering transcription or translation thereof. Such molecules include, but are not limited to, antibodies that specifically bind the polypeptide, antisense nucleic acids or double-stranded RNA (dsRNA) such as RNAi, that alter expression of the polypeptide, antibodies, peptide mimetics and other such compounds.

Antibodies are provided, including polyclonal and monoclonal antibodies that specifically bind to a polypeptide gene product provided herein. An antibody can be a monoclonal antibody, and the antibody can specifically bind to the polypeptide. The polypeptide and domains, fragments, homologs and derivatives thereof can be used as immunogens to generate antibodies that specifically bind such immunogens. Such antibodies include but are not limited to polyclonal, monoclonal, chimeric, single chain, Fab fragments, and an Fab expression library. In a specific embodiment, antibodies to human polypeptides are produced. Methods for monoclonal and polyclonal antibody production are known in the art. Antibody fragments that specifically bind to the polypeptide or epitopes thereof can be generated by techniques known in the art. For example, such fragments include but are not limited to: the F(ab′)2 fragment, which can be produced by pepsin digestion of the antibody molecule; the Fab′ fragments that can be generated by reducing the disulfide bridges of the F(ab′)2 fragment, the Fab fragments that can be generated by treating the antibody molecular with papain and a reducing agent, and Fv fragments.

Peptide analogs are commonly used in the pharmaceutical industry as non-peptide drugs with properties analogous to those of the template peptide. These types of non-peptide compounds are termed peptide mimetics or peptidomimetics (Luthman et al., A Textbook of Drug Design and Development, 14:386-406, 2nd Ed., Harwood Academic Publishers (1996); Joachim Grante (1994) Angew. Chem. Int. Ed. Engl., 33:1699-1720; Fauchere (1986) J. Adv. Drug Res., 15:29; Veber and Freidinger (1985) TINS, p. 392; and Evans et al. (1987) J. Med. Chem. 30:1229). Peptide mimetics that are structurally similar to therapeutically useful peptides can be used to produce an equivalent or enhanced therapeutic or prophylactic effect. Preparation of peptidomimetics and structures thereof are known to those of skill in this art.

### Prognosis and Diagnosis

Polypeptide products of the coding sequences (e.g., genes) listed herein can be detected in diagnostic methods, such as diagnosis of tumors and other diseases or disorders. Such methods can be used to detect, prognose, diagnose, or monitor various conditions, diseases, and disorders. Exemplary compounds that can be used in such detection methods include polypeptides such as antibodies or fragments thereof that specifically bind to the polypeptides listed herein, and oligonucleotides such as DNA probes or primers that specifically bind oligonucleotides such as RNA encoded by the nucleic acids provided herein.

A set of one or more, or two or more compounds for detection of markers containing a particular nucleotide sequence, complements thereof, fragments thereof, or polypeptides encoded thereby, can be selected for any of a variety of assay methods provided herein. For example, one or more, or two or more such compounds can be selected as diagnostic or prognostic indicators. Methods for selecting such compounds and using such compounds in assay methods such as diagnostic and prognostic indicator applications are known in the art. For example, the Tables provided herein list a modified t statistic associated with each marker, where the modified t statistic indicate the ability of the associated marker to indicate (by presence or absence of the marker, according to the modified t statistic) the presence or absence of a particular cell type in a prostate sample.

In another embodiment, marker selection can be performed by considering both modified t statistics and expected intensity of the signal for a particular marker. For example, markers can be selected that have a strong signal in a cell type whose presence or absence is to be determined, and also have a sufficiently large modified t statistic for gene expression in that cell type. Also, markers can be selected that have little or no signal in a cell type whose presence or absence is to be determined, and also have a sufficiently large negative modified t statistic for gene expression in that cell type.

Exemplary assays include immunoassays such as competitive and non-competitive assay systems using techniques such as western blots, radioimmunoassays, ELISA (enzyme linked immunosorbent assay), sandwich immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Other exemplary assays include hybridization assays which can be carried out by a method by contacting a sample containing nucleic acid with a nucleic acid probe, under conditions such that specific hybridization can occur, and detecting or measuring any resulting hybridization.

Kits for diagnostic use are also provided, that contain in one or more containers an anti-polypeptide antibody, and, optionally, a labeled binding partner to the antibody. A kit is also provided that includes in one or more containers a nucleic acid probe capable of hybridizing to the gene-encoding nucleic acid. In a specific embodiment, a kit can include in one or more containers a pair of primers (e.g., each in the size range of 6-30 nucleotides) that are capable of priming amplification. A kit can optionally further include in a container a predetermined amount of a purified control polypeptide or nucleic acid.

The kits can contain packaging material that is one or more physical structures used to house the contents of the kit, such as invention nucleic acid probes or primers, and the like. The packaging material is constructed by well known methods, and can provide a sterile, contaminant-free environment. The packaging material has a label which indicates that the compounds can be used for detecting a particular oligonucleotide or polypeptide. The packaging materials employed herein in relation to diagnostic systems are those customarily utilized in nucleic acid or protein-based diagnostic systems. A package is to a solid matrix or material such as glass, plastic, paper, foil, and the like, capable of holding within fixed limits an isolated nucleic acid, oligonucleotide, or primer of the present invention. Thus, for example, a package can be a glass vial used to contain milligram quantities of a contemplated nucleic acid, oligonucleotide or primer, or it can be a microtiter plate well to which microgram quantities of a contemplated nucleic acid probe have been operatively affixed. The kits also can include instructions for use, which can include a tangible expression describing the reagent concentration or at least one assay method parameter, such as the relative amounts of reagent and sample to be admixed, maintenance time periods for reagent/sample admixtures, temperature, buffer conditions, and the like.

### Pharmaceutical Compositions and Modes of Administration

Pharmaceutical compositions containing the identified compounds that modulate expression of a gene or bind to a gene product are provided herein. Also provided are combinations of such a compound and another treatment or compound for treatment of a disease or disorder, such as a chemotherapeutic compound.

Expression modulator or binding compound and other compounds can be packaged as separate compositions for administration together or sequentially or intermittently. Alternatively, they can be provided as a single composition for administration or as two compositions for administration as a single composition. The combinations can be packaged as kits.

Compounds and compositions provided herein can be formulated as pharmaceutical compositions, for example, for single dosage administration. The concentrations of the compounds in the formulations are effective for delivery of an amount, upon administration, that is effective for the intended treatment. In certain embodiments, the compositions are formulated for single dosage administration. To formulate a composition, the weight fraction of a compound or mixture thereof is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds can be formulated as the sole pharmaceutically active ingredient in the composition or can be combined with other active ingredients. The active compound is included in the pharmaceutically acceptable carrier in an amount sufficient to exert a therapeutically useful effect in the absence of undesirable side effects on the subject treated. The therapeutically effective concentration can be determined empirically by testing the compounds in known in vitro and in vivo systems. The concentration of active compound in the drug composition depends on absorption, inactivation and excretion rates of the active compound, the physicochemical characteristics of the compound, the dosage schedule, and amount administered as well as other factors known to those of skill in the art. Pharmaceutically acceptable derivatives include acids, salts, esters, hydrates, solvates and prodrug forms. The derivative can be selected such that its pharmacokinetic properties are superior to the corresponding neutral compound. Compounds are included in an amount effective for ameliorating or treating the disorder for which treatment is contemplated.

Formulations suitable for a variety of administrations such as perenteral, intramuscular, subcutaneous, alimentary, transdermal, inhaling and other known methods of administration, are known in the art. The pharmaceutical compositions can also be administered by controlled release means and/or delivery devices as known in the art. Kits containing the compositions and/or the combinations with instructions for administration thereof are provided. The kit can further include a needle or syringe, which can be packaged in sterile form, for injecting the complex, and/or a packaged alcohol pad. Instructions are optionally included for administration of the active agent by a clinician or by the patient.

The compounds can be packaged as articles of manufacture containing packaging material, a compound or suitable derivative thereof provided herein, which is effective for treatment of a diseases or disorders contemplated herein, within the packaging material, and a label that indicates that the compound or a suitable derivative thereof is for treating the diseases or disorders contemplated herein. The label can optionally include the disorders for which the therapy is warranted.

### Methods of Treatment

The compounds provided herein can be used for treating or preventing diseases or disorders in an animal, such as a mammal, including a human. In one embodiment, the method includes administering to a mammal an effective amount of a compound that modulates the expression of a particular gene (e.g., a gene listed herein) or a compound that binds to a product of a gene, whereby the disease or disorder is treated or prevented. Exemplary inhibitors provided herein are those identified by the screening assays. In addition, antibodies and antisense nucleic acids or double-stranded RNA (dsRNA), such as RNAi, are contemplated.

In a specific embodiment, as described hereinabove, gene expression can be inhibited by antisense nucleic acids. The therapeutic or prophylactic use of nucleic acids of at least six nucleotides, up to about 150 nucleotides, that are antisense to a gene or cDNA is provided. The antisense molecule can be complementary to all or a portion of the gene. For example, the oligonucleotide is at least 10 nucleotides, at least 15 nucleotides, at least 100 nucleotides, or at least 125 nucleotides. The oligonucleotides can be DNA or RNA or chimeric mixtures or derivatives or modified versions thereof, single-stranded or double-stranded. The oligonucleotide can be modified at the base moiety, sugar moiety, or phosphate backbone. The oligonucleotide can include other appending groups such as peptides, or agents facilitating transport across the cell membrane, hybridization-triggered cleavage agents or intercalating agents.

RNA interference (RNAi) (see, e.g., Chuang et al. (2000) Proc. Natl. Acad. Sci. U.S.A. 97:4985) can be employed to inhibit the expression of a nucleic acid. Interfering RNA (RNAi) fragments, such as double-stranded (ds) RNAi, can be used to generate loss-of-gene function. Methods relating to the use of RNAi to silence genes in organisms including, mammals, C. elegans, Drosophila and plants, and humans are known. Double-stranded RNA (dsRNA)-expressing constructs are introduced into a host, such as an animal or plant using, a replicable vector that remains episomal or integrates into the genome. By selecting appropriate sequences, expression of dsRNA can interfere with accumulation of endogenous mRNA. RNAi also can be used to inhibit expression in vitro. Regions include at least about 21 (or 21) nucleotides that are selective (i.e., unique) for the selected gene are used to prepare the RNAi. Smaller fragments of about 21 nucleotides can be transformed directly (i.e., in vitro or in vivo) into cells; larger RNAi dsRNA molecules can be introduced using vectors that encode them. dsRNA molecules are at least about 21 bp long or longer, such as 50, 100, 150, 200 and longer. Methods, reagents and protocols for introducing nucleic acid molecules in to cells in vitro and in vivo are known to those of skill in the art.

In an exemplary embodiment, nucleic acids that include a sequence of nucleotides encoding a polypeptide of a gene as listed herein can be administered to promote polypeptide function, by way of gene therapy. Gene therapy refers to therapy performed by administration of a nucleic acid to a subject. In this embodiment, the nucleic acid produces its encoded protein that mediates a therapeutic effect by promoting polypeptide function. Any of the methods for gene therapy available in the art can be used (see, Goldspiel et al., Clinical Pharmacy 12:488-505 (1993); Wu and Wu, Biotherapy 3:87-95 (1991); Tolstoshev, An. Rev. Pharmacol. Toxicol. 32:573-596 (1993); Mulligan, Science 260:926-932 (1993); and Morgan and Anderson, An. Rev. Biochem. 62:191-217 (1993); TIBTECH 11 (5):155-215 (1993).

In some embodiments, vaccines based on the genes and polypeptides provided herein can be developed. For example genes can be administered as DNA vaccines, either single genes or combinations of genes. Naked DNA vaccines are generally known in the art. Methods for the use of genes as DNA vaccines are well known to one of ordinary skill in the art, and include placing a gene or portion of a gene under the control of a promoter for expression in a patient with cancer. The gene used for DNA vaccines can encode full-length proteins, but can encode portions of the proteins including peptides derived from the protein. For example, a patient can be immunized with a DNA vaccine comprising a plurality of nucleotide sequences derived from a particular gene. In another embodiment, it is possible to immunize a patient with a plurality of genes or portions thereof. Without being bound by theory, expression of the polypeptide encoded by the DNA vaccine, cytotoxic T-cells, helper T-cells and antibodies are induced that recognize and destroy or eliminate cells expressing the proteins provided herein.

DNA vaccines can include a gene encoding an adjuvant molecule with the DNA vaccine. Such adjuvant molecules include cytokines that increase the immunogenic response to the polypeptide encoded by the DNA vaccine. Additional or alternative adjuvants are known to those of ordinary skill in the art and find use in the invention.

### Animal Models and Transgenics

Also provided herein, the nucleotide the genes, nucleotide molecules and polypeptides disclosed herein find use in generating animal models of cancers, such as lymphomas and carcinomas. As is appreciated by one of ordinary skill in the art, when one of the genes provided herein is repressed or diminished, gene therapy technology wherein antisense RNA directed to the gene will also diminish or repress expression of the gene. An animal generated as such serves as an animal model that finds use in screening bioactive drug candidates. In another embodiment, gene knockout technology, for example as a result of homologous recombination with an appropriate gene targeting vector, will result in the absence of the protein. When desired, tissue-specific expression or knockout of the protein can be accomplished using known methods.

It is also possible that a protein is overexpressed in cancer. As such, transgenic animals can be generated that overexpress the protein. Depending on the desired expression level, promoters of various strengths can be employed to express the transgene. Also, the number of copies of the integrated transgene can be determined and compared for a determination of the expression level of the transgene. Animals generated by such methods find use as animal models and are additionally useful in screening for bioactive molecules to treat cancer.

### Computer Programs and Methods

The various techniques, methods, and aspects of the methods provided herein can be implemented in part or in whole using computer-based systems and methods. In another embodiment, computer-based systems and methods can be used to augment or enhance the functionality described above, increase the speed at which the functions can be performed, and provide additional features and aspects as a part of or in addition to those of the invention described elsewhere in this document. Various computer-based systems, methods and implementations in accordance with the above-described technology are presented below.

A processor-based system can include a main memory, such as random access memory (RAM), and can also include a secondary memory. The secondary memory can include, for example, a hard disk drive and/or a removable storage drive, representing a floppy disk drive, a magnetic tape drive, or an optical disk drive. The removable storage drive reads from and/or writes to a removable storage medium. Removable storage medium refers to a floppy disk, magnetic tape, optical disk, and the like, which is read by and written to by a removable storage drive. As will be appreciated, the removable storage medium can comprise computer software and/or data.

In alternative embodiments, the secondary memory may include other similar means for allowing computer programs or other instructions to be loaded into a computer system. Such means can include, for example, a removable storage unit and an interface. Examples of such can include a program cartridge and cartridge interface (such as the found in video game devices), a movable memory chip (such as an EPROM or PROM) and associated socket, and other removable storage units and interfaces, which allow software and data to be transferred from the removable storage unit to the computer system.

The computer system can also include a communications interface. Communications interfaces allow software and data to be transferred between computer system and external devices. Examples of communications interfaces can include a modem, a network interface (such as, for example, an Ethernet card), a communications port, a PCMCIA slot and card, and the like. Software and data transferred via a communications interface are in the form of signals, which can be electronic, electromagnetic, optical or other signals capable of being received by a communications interface. These signals are provided to communications interface via a channel capable of carrying signals and can be implemented using a wireless medium, wire or cable, fiber optics or other communications medium. Some examples of a channel can include a phone line, a cellular phone link, an RF link, a network interface, and other communications channels.

In this document, the terms computer program medium and computer usable medium are used to refer generally to media such as a removable storage device, a disk capable of installation in a disk drive, and signals on a channel. These computer program products are means for providing software or program instructions to a computer system.

Computer programs (also called computer control logic) are stored in main memory and/or secondary memory. Computer programs can also be received via a communications interface. Such computer programs, when executed, permit the computer system to perform the features of the invention as discussed herein. In particular, the computer programs, when executed, permit the processor to perform the features of the invention. Accordingly, such computer programs represent controllers of the computer system.

In an embodiment where the elements are implemented using software, the software may be stored in, or transmitted via, a computer program product and loaded into a computer system using a removable storage drive, hard drive or communications interface. The control logic (software), when executed by the processor, causes the processor to perform the functions of the invention as described herein.

In another embodiment, the elements are implemented in hardware using, for example, hardware components such as PALs, application specific integrated circuits (ASICs) or other hardware components Implementation of a hardware state machine so as to perform the functions described herein will be apparent to person skilled in the relevant art(s). In yet another embodiment, elements are implanted using a combination of both hardware and software.

In another embodiment, the computer-based methods can be accessed or implemented over the World Wide Web by providing access via a Web Page to the methods of the invention. Accordingly, the Web Page is identified by a Universal Resource Locator (URL). The URL denotes both the server machine and the particular file or page on that machine. In this embodiment, it is envisioned that a consumer or client computer system interacts with a browser to select a particular URL, which in turn causes the browser to send a request for that URL or page to the server identified in the URL. The server can respond to the request by retrieving the requested page and transmitting the data for that page back to the requesting client computer system (the client/server interaction can be performed in accordance with the hypertext transport protocol (HTTP)). The selected page is then displayed to the user on the client's display screen. The client may then cause the server containing a computer program of the invention to launch an application to, for example, perform an analysis according to the methods provided herein.

### Prostate-Associated Genes

Provided herein are probe and gene sequences that can be indicative of the presence and/or absence of prostate cancer in a subject. Also provided herein are probe and gene sequences that can be indicative of presence and/or absence of benign prostatic hyperplasia (BPH) in a subject. Also provided herein are probe and gene sequences that can be indicative of a prognosis of prostate cancer, where such a prognosis can include likely relapse of prostate cancer, likely aggressiveness of prostate cancer, likely indolence of prostate cancer, likelihood of survival of the subject, likelihood of success in treating prostate cancer, condition in which a particular treatment regimen is likely to be more effective than another treatment regimen, and combinations thereof. In one embodiment, the probe and gene sequences can be indicative of the likely aggressiveness or indolence of prostate cancer.

As provided in the methods and Tables herein, probes have been identified that hybridize to one or more nucleic acids of a prostate sample at different levels according to the presence or absence of prostate tumor, BPH and stroma in the sample. The probes provided herein are listed in conjunction with modified t statistics that represent the ability of that particular probe to indicate the presence or absence of a particular cell type in a prostate sample. Use of modified t statistics for such a determination is described elsewhere herein, and general use of modified t statistics is known in the art. Accordingly, provided herein are nucleotide sequences of probes that can be indicative of the presence or absence of prostate tumor and/or BPH cells, and also can be indicative of the likelihood of prostate tumor relapse in a subject.

Also provided in the methods and Tables herein are nucleotide and predicted amino acid sequences of genes and gene products associated with the probes provided herein. Accordingly, as provided herein, detection of gene products (e.g., mRNA or protein) or other indicators of gene expression, can be indicative of the presence or absence of prostate tumor and/or BPH cells, and also can be indicative of the likelihood of prostate tumor relapse in a subject. As with the probe sequences, the nucleotide and amino acid sequences of these gene products are listed in conjunction with modified t statistics that represent the ability of that particular gene product or indicator thereof to indicate the presence or absence of a particular cell type in a prostate sample.

Methods for determining the presence of prostate tumor and/or BPH cells, the likelihood of prostate tumor relapse in a subject, the likelihood of survival of prostate cancer, the aggressiveness of prostate tumor, the indolence of prostate tumor, survival, and other prognoses of prostate tumor, can be performed in accordance with the teachings and examples provided herein. Also provided herein, a set of probes or gene products can be selected according to their modified t statistic for use in combination (e.g., for use in a microarray) in methods of determining the presence of prostate tumor and/or BPH cells, and/or the likelihood of prostate tumor relapse in a subject.

Also provided herein, the gene products identified as present at increased levels in prostate cancer or in subjects with likely relapse of cancer, can serve as targets for therapeutic compounds and methods. For example an antibody or siRNA targeted to a gene product present at increased levels in prostate cancer can be administered to a subject to decrease the levels of that gene product and to thereby decrease the malignancy of tumor cells, the aggressiveness of a tumor, indolence of a tumor, survival, or the likelihood of tumor relapse. Methods for providing molecules such as antibodies or siRNA to a subject to decrease the level of gene product in a subject are provided herein or are otherwise known in the art.

In some embodiments, gene products identified as present at decreased levels in prostate cancer or in subjects with likely relapse of cancer, can serve as subjects for therapeutic compounds and methods. For example a nucleic acid molecule, such as a gene expression vector encoding a particular gene, can be administered to a individual with decreased levels of the particular gene product to increase the levels of that gene product and to thereby decrease the malignancy of tumor cells, the aggressiveness of a tumor, indolence of a tumor, likelihood of survival, or the likelihood of tumor relapse. Methods for providing gene expression vectors to a subject to increase the level of gene product in a subject are provided herein or are otherwise known in the art.

As used herein, the term “prostate cancer signature” refers to genes that exhibit altered expression (e.g., increased or decreased expression) with prostate cancer as compared to control levels of expression (e.g., in normal prostate tissue). Genes included in a prostate cancer signature can include any of those listed in the tables presented herein (e.g., Tables 3 and 4). For example, one or more (e.g., two, three, four, five, six, seven, eight nine, ten, 15, 20, 25, 30, 35, 40, 45, 50, 55, 60, 65, or more) of the genes listed in Table 3 can be are present in a prostate tissue sample (e.g., a prostate tissue sample containing normal stroma, prostate cancer cells, or both) at a level greater than or less than the level observed in normal, non-cancerous prostate tissue. In some cases, a prostate cancer signature can be a gene expression profile in which at least 10, 20, 30, 40, 50, 60, 70, 80, 90, or 100 percent of the genes listed in a table herein (e.g., Table 3 or Table 4) are expressed at a level greater than or less than their corresponding control levels in non-cancerous tissue.

As used herein, the terms “prostate cell-type predictor” genes and “prostate tissue predictor” genes refer to genes that can, based on their expression levels, serve as indicators as to whether a particular sample of prostate tissue contains particular cell types (e.g., prostate cancer cells, normal stromal cells, epithelial cells of benign prostate hyperplasia, or epithelial cells of dilated cystic glands). Such genes also can indicate the relative amounts of such cell types within the prostate tissue sample.

In some embodiments, this document features methods for identifying a subject as having or not having prostate cancer, comprising: (a) providing a prostate tissue sample from the subject; (b) measuring the level of expression for prostate cancer signature genes in the sample; (c) comparing the measured expression levels to reference expression levels for the prostate cancer signature genes; and (d) if the measured expression levels are significantly greater or less than the reference expression levels, identifying the subject as having prostate cancer, and if the measured expression levels are not significantly greater or less than the reference expression levels, identifying the subject as not having prostate cancer. The prostate tissue sample may not include tumor cells, or the prostate tissue sample may include tumor cells and stromal cells. The prostate cancer signature genes can be selected from the genes listed in the Tables herein (e.g., in Table 3 or Table 4). The method can include determining whether measured expression levels for ten or more prostate cancer signature genes are significantly greater or less than reference expression levels for the ten or more prostate cancer signature genes, and classifying the subject as having prostate cancer that is likely to relapse if the measured expression levels are significantly greater or less than the reference expression levels, or classifying the subject as having prostate cancer not likely to relapse if the measured expression levels are not significantly greater or less than the reference expression levels. The ten or more prostate cancer signature genes can be selected from the genes listed in Table 3 or Table 4 herein, for example. The method can include determining whether measured expression levels for twenty or more prostate cancer signature genes are significantly greater or less than reference expression levels for the twenty or more prostate cancer signature genes, and classifying the subject as having prostate cancer that is likely to relapse if the measured expression levels are significantly greater or less than the reference expression levels, or classifying the subject as having prostate cancer not likely to relapse if the measured expression levels are not significantly greater or less than the reference expression levels. The twenty or more prostate cancer signature genes can be selected from the genes listed in Table 3 or Table 4 herein, for example.

This document also features methods for determining the prognosis of a subject diagnosed as having prostate cancer, comprising: (a) providing a prostate tissue sample from the subject; (b) measuring the level of expression for prostate cancer signature genes in the sample; (c) comparing the measured expression levels to reference expression levels for the prostate cancer signature genes; and (d) if the measured expression levels are not significantly greater or less than the reference expression levels, identifying the subject as having a relatively better prognosis than if the measured expression levels are significantly greater or less than the reference expression levels, or if the measured expression levels are significantly greater or less than the reference expression levels, identifying the subject as having a relatively worse prognosis than if the measured expression levels are not significantly greater or less than the reference expression levels. The prostate tissue sample may not include tumor cells, or the prostate tissue sample may include tumor cells and stromal cells. The prostate cancer signature genes can be selected from the genes listed in the Tables herein (e.g., Table 8A or 8B).

In addition, this document provides methods for identifying a subject as having or not having prostate cancer, comprising: (a) providing a prostate tissue sample from the subject, wherein the sample comprises prostate stromal cells; (b) measuring expression levels for one or more genes in the stromal cells, wherein the one or more genes are prostate cancer signature genes; (c) comparing the measured expression levels to reference expression levels for the one or more genes, wherein the reference expression levels are determined in stromal cells from non-cancerous prostate tissue; and (d) if the measured expression levels are significantly greater or less than the reference expression levels, identifying the subject as having prostate cancer, and if the measured expression levels are not significantly greater or less than the reference expression levels, identifying the subject as not having prostate cancer. The prostate tissue sample may not include tumor cells, or the prostate tissue sample may include tumor cells and stromal cells. The prostate cancer signature genes can be selected from the genes listed in Table 3 or Table 4 herein, for example.

This document also provides methods for determining a prognosis for a subject diagnosed as having prostate cancer, comprising: (a) providing a prostate tissue sample from the subject, wherein the sample comprises prostate stromal cells; (b) measuring expression levels for one or more genes in the stromal cells, wherein the one or more genes are prostate cancer signature genes; (c) comparing the measured expression levels to reference expression levels for the one or more genes, wherein the reference expression levels are determined in stromal cells from non-cancerous prostate tissue; and (d) if the measured expression levels are not significantly greater or less than the reference expression levels, identifying the subject as having a relatively better prognosis than if the measured expression levels are significantly greater or less than the reference expression levels, or if the measured expression levels are significantly greater or less than the reference expression levels, identifying the subject as having a relatively worse prognosis than if the measured expression levels are not significantly greater or less than the reference expression levels. The prostate tissue sample may not include tumor cells, or the prostate tissue sample may include tumor cells and stromal cells. The prostate cancer signature genes can be selected from the genes listed in the tables herein (e.g., Table 3 or Table 4).

Further, this document features a method for identifying a subject as having or not having prostate cancer, comprising: (a) providing a prostate tissue sample from the subject; (b) measuring expression levels for one or more prostate cell-type predictor genes in the sample; (c) determining the percentages of tissue types in the sample based on the measured expression levels; (d) measuring expression levels for one more prostate cancer signature genes in the sample; (e) determining a classifier based on the percentages of tissue types and the measured expression levels; and (f) if the classifier falls into a predetermined range of prostate cancer classifiers, identifying the subject as having prostate cancer, or if the classifier does not fall into the predetermined range, identifying the subject as not having prostate cancer. Steps (b) and (d) can be carried out simultaneously.

This document also features a method for determining a prognosis for a subject diagnosed with and treated for prostate cancer, comprising: (a) providing a prostate tissue sample from the subject; (b) measuring expression levels for one or more prostate tissue predictor genes in the sample; (c) determining the percentages of tissue types in the sample based on the measured expression levels; (d) measuring expression levels for one more prostate cancer signature genes in the sample; (e) determining a classifier based on the percentages of tissue types and the measured expression levels; and (f) if the classifier falls into a predetermined range of prostate cancer relapse classifiers, identifying the subject as being likely to relapse, or if the classifier does not fall into the predetermined range, identifying the subject as not being likely to relapse. Steps (b) and (d) are carried out simultaneously.

In some embodiments, methods as described herein can be used for identifying the proportion of two or more tissue types in a tissue sample. Such methods can include, for example: (a) using a set of other samples of known tissue proportions from a similar anatomical location as the tissue sample in an animal or plant, wherein at least two of the other samples do not contain the same relative content of each of the two or more cell types; (b) measuring overall levels of one or more gene expression or protein analytes in each of the other samples; (c) determining the regression relationship between the relative proportion of each tissue type and the measured overall levels of each gene expression or protein analyte in the other samples; (d) selecting one or more analytes that correlate with tissue proportions in the other samples; (e) measuring overall levels of one or more of the analytes in step (d) in the tissue sample; (f) matching the level of each analyte in the tissue sample with the level of the analyte in step (d) to determine the predicted proportion of each tissue type in the tissue sample; and (g) selecting among predicted tissue proportions for the tissue sample obtained in step (f) using either the median or average proportions of all the estimates. The tissue sample can contain cancer cells (e.g., prostate cancer cells).

Methods described herein can be used for comparing the levels of two or more analytes predicted by one or more methods to be associated with a change in a biological phenomenon in two sets of data each containing more than one measured sample. Such methods can comprise: (a) selecting only analytes that are assayed in both sets of data; (b) ranking the analytes in each set of data using a comparative method such as the highest probability or lowest false discovery rate associated with the change in the biological phenomenon; (c) comparing a set of analytes in each ranked list in step (b) with each other, selecting those that occur in both lists, and determining the number of analytes that occur in both lists and show a change in level associated with the biological phenomenon that is in the same direction; and (d) calculating a concordance score based on the probability that the number of comparisons would show the observed number of change in the same direction, at random. In step (a), the length of each list can be varied to determine the maximum concordance score for the two ranked lists.

The invention will be further described in the following examples, which do not limit the scope of the invention described in the claims.

### EXAMPLES

### Example 1

### Diagnosis of Prostate Cancer without Tumor Cells Using Differentially Expressed Genes in Stroma Adjacent to Tumors

Over one million prostate biopsies are performed in the U.S. every year. Pathology examination is not definitive in a significant percentage of cases, however, due to the presence of equivocal structures or continuing clinical suspicion. To investigate gene expression changes in the tumor microenvironment vs. normal stroma, gene expression profiles from 15 volunteer biopsy specimens were compared to profiles from 13 specimens containing largely tumor-adjacent stroma. As described below, more than a thousand significant expression changes were identified and filtered to eliminate possible age-related genes, as well as genes that also are expressed at detectable levels in tumor cells. A stroma-specific classifier was constructed based on the 114 remaining unique candidate genes (131 Affymetrix probe sets). The classifier was tested on 380 independent cases, including 255 tumor-bearing cases and 125 non-tumor cases (normal biopsies, normal autopsies, remote stroma as well as pure tumor adjacent stroma). The classifier predicted the tumor status of patients with an average accuracy of 97.4% (sensitivity=98.0% and specificity=89.7%), whereas a randomly generated and trained classifier had no diagnostic value. These results indicate that the prostate cancer microenvironment exhibits reproducible changes useful for categorizing stroma as “presence of tumor” and “non-presence of tumor.”

Prostate Cancer Patients Samples and Expression Analysis: Datasets 1 and 2 (Table 1) were obtained using post-prostatectomy frozen tissue samples. All tissues, except where noted, were collected at surgery and escorted to pathology for expedited review, dissection, and snap freezing in liquid nitrogen. RNA for expression analysis was prepared directly from frozen tissue following dissection of OCT (optimum cutting temperature compound) blocks with the aid of a cryostat. For expression analysis, 50 micrograms (10 micrograms for biopsy tissue) of total RNA samples were processed for hybridization to Affymetrix GeneChips.

Dataset 1 consists of 109 post-prostatectomy frozen tissue samples from 87 patients. Twenty-two cases were analyzed twice using one sample from a tumor-enriched specimen and one sample from a non-tumor specimen (more than 1.5 cm away from the tumor), usually the contralateral lobe. In addition, Dataset 1 contains 27 prostate biopsy specimens obtained as fresh snap frozen biopsy cores from 18 normal participants in a clinical trial to evaluate the role of Difluoromethylornithine (DFMO) to decrease the prostate size of normal men (Simoneau et al. (2008) Cancer Epidemiol. Biomarkers Prey. 17:292-299). Finally, Dataset 1 contains 13 cases of normal prostates obtained from the rapid autopsy program of the Sun Health Research Institute, from subjects with an average age of 82 years.

Dataset 2 contains 136 samples from 82 patients, where 54 cases were analyzed as pairs of tumor-enriched samples and, for most cases, non-tumor tissue obtained from the same OCT block as tumor-adjacent tissue. This series includes specimens for which expression coefficients were validated (Stuart et al. (2004) Proc. Natl. Acad. Sci. U.S.A. 101:615-620).

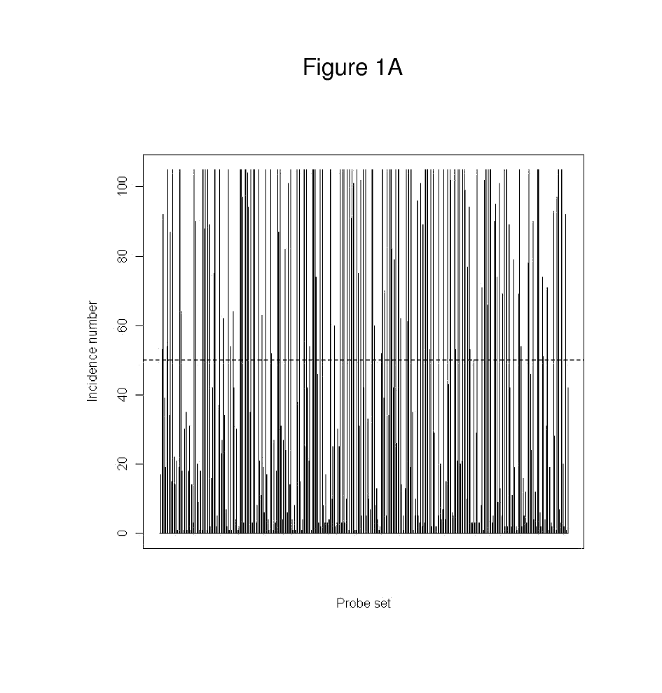

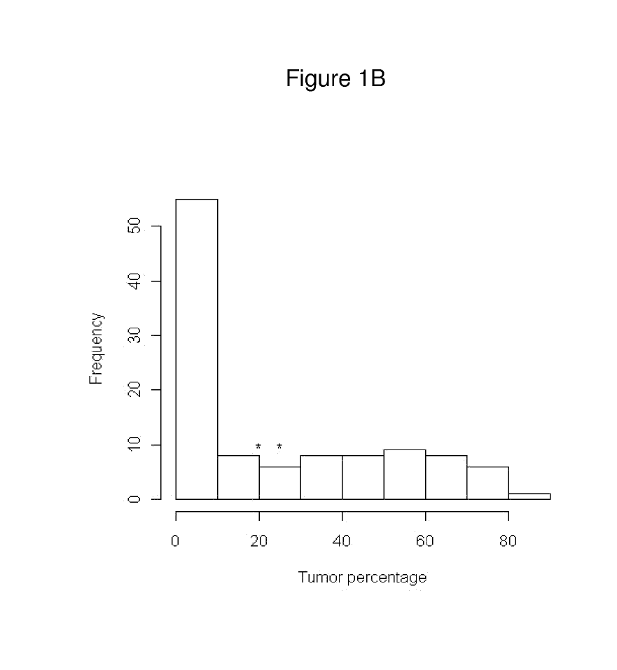

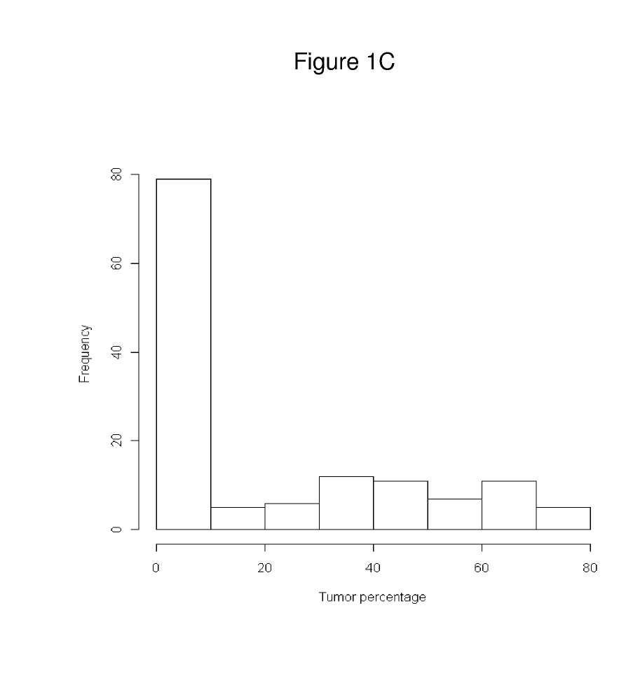

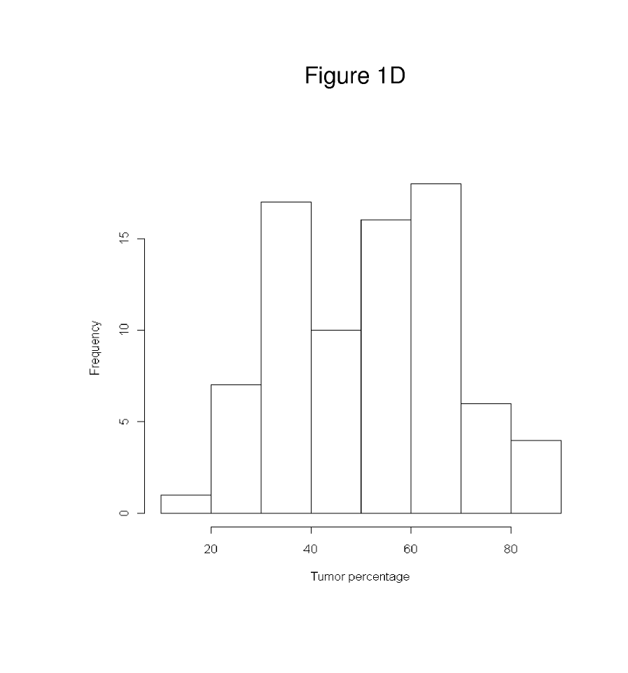

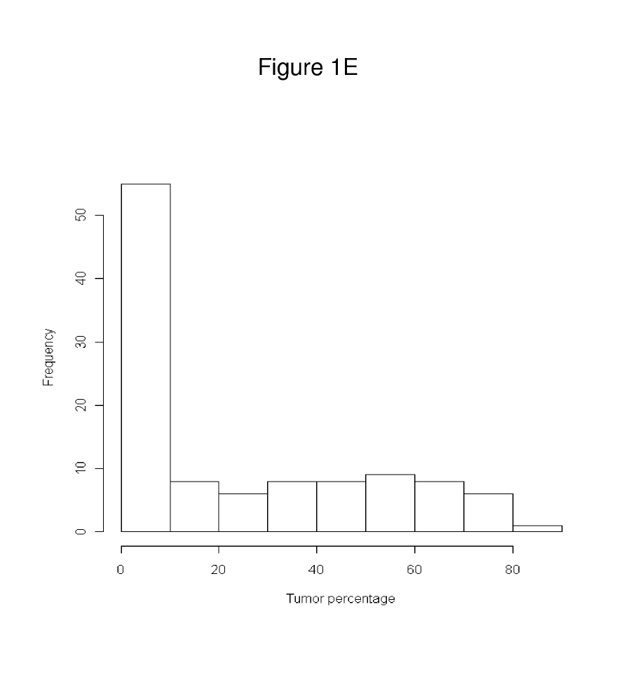

Expression analysis for Datasets 1 and 2 was carried out using Affymetrix U133Plus2 and U133A GeneChips, respectively; the expression data are publicly available at GEO database on the World Wide Web at ncbi.nlm.nih gov/geo, with accession numbers GSE17951 (Dataset 1) and GSE8218 (Dataset 2). For both datasets, cell type distributions for the four principal cell types (tumor epithelial cells, stroma cells, epithelial cells of BPH, and epithelial cells of dilated cystic glands) were determined from frozen sections prepared immediately before and after the sections pooled for RNA preparation by three (Dataset 1) or four (Dataset 2) pathologists whose estimates were averaged as described (Stuart et al., supra). The distributions of tumor percentage for Dataset 1 and 2 are shown in FIGS. 1B and 1C.

Dataset 3 consists of a published series (Stephenson et al. (2005) Cancer 104:290-298) of 79 cases for which expression data were measured with Affymetrix U133A chips. The cell composition was not documented at the time of data collection. Cell composition was estimated using multigene signatures that are invariant with tumor surgical pathology parameters of Gleason and stage by the CellPred program (World Wide Web at webarraydb.org), which confirmed that all 79 samples included tumor cells, with tumor content ranging from 24% to 87% (FIG. 1D). Dataset 4 includes 57 samples from 44 patients, including 13 tumor-adjacent stroma samples and 44 tumor-bearing samples. Gene expression in these 57 samples was measured with Affymetrix U133A GeneChips. Tumor percentage (ranging from 0% to 80%, FIG. 1E) was approximated using the CellPred program.

Dataset 5 consists of 4 pooled normal stromal samples and 12 tumor samples gleaned by Laser Capture Micro dissection (LCM) using frozen tissue samples. Each pooled normal stroma sample was pooled from two LCM captured stroma samples from specimens from which no tumor was recovered in the surgical samples available for the research protocol described herein, whereas tumor samples were LCM-captured prostate cancer cells. Gene expression in these 16 samples (using 10 micrograms of total RNA) was measured using Affymetrix U133Plus2 chips.

Compared to U133A (with ˜22,000 probe sets) used for Datasets 2, 3 and 4, the U133Plus2 platform used for Datasets 1 and 5 had about 30,000 more probe sets. To attain an analysis across multiple datasets, only the probes common to these two platforms were used, i.e., only about 22,000 common probe sets in each Dataset were considered. First, Dataset 1 was quantile-normalized using function ‘normalizeQuantiles( )’ of LIMMA routine (Dalgaard (2002) Statistics and Computing: Introductory Statistics with R, p. 260, Springer-Verlag Inc., New York. Datasets 2-5 were then quantile-normalized by referencing normalized Dataset 1 with a modified function ‘REFnormalizeQuantiles( )’ which is available from ZJ.

TABLE 1
Datasets used in the study¹

| | | | | Array: | |
|---|---|---|---|---|---|
| | | | | Tumor/ | |
| | | Subj. | Array | Nontumor/ | |
| Data | Platform | Num. | Num. | Normal | Ref. |
| 1 | U133Plus2 | P = 87 | 109 | 69/40/0 | GSE17951 |
| Training + | | B = 18 | 27 | 0/0/27 | |
| Test | | A = 13 | 13 | 0/0/13 | |
| 2 | U133A | P = 82 | 136 | 65/71/0 | GSE08218 |
| Test | | | | | |
| 3 | U133A | P = 79 | 79 | 79/0/0 | Stephenson |
| Test | | | | | et al., supra |
| 4 | U133A | P = 44 | 57 | 44/13/0 | http://www.ebi.- |
| Test | | | | | ac.uk/microarray- |
| | | | | | as/ae/browse.- |
| | | | | | html?keywords= |
| | | | | | E-TABM-26 |
| 5 | U133P2 | L = 20 | 16 | 12/0/4 | GSE17951 |
| Test | | | | | |

¹P, B, A, and L represent patient, normal biopsy, normal rapid autopsy, and LCM, respectively. Datasets 1 and 2 were collected from five participating institutions in San Diego County, CA. Demographic, Pathology, and clinical values are individually recorded (Shadow charts) and maintained in the UCI SPECS consortium database including tracking sheets of elapsed times following surgery during sample handling.

Statistical tools implemented in R.: The Linear Models for Microarray Data (LIMMA package from Bioconductor, on the World Wide Web at bioconductor.org) was used to detect differentially expressed genes. Prediction Analysis of Microarray (PAM, implemented by the PAMR package from Bioconductor) was used to develop an expression-based classifier from training set and then applied to the test sets without any change (Guo et al. (2007) Biostatistics 8:86-100). Fisher's Exact Test was used to demonstrate the efficiency of the classifier when it was tested on remote stroma versus tumor adjacent stroma. Fisher's test was used instead of chi-square because chi-square test is not suitable when the expected values in any of the cells of the table are below 10. All statistical analysis was done using R language (World Wide Web at r-project.org).

Multiple Linear Regression Model: A multiple linear regression (MLR) model was used to describe the observed Affymetrix intensity of a gene as the summation of the contributions from different types of cells given the pathological cell constitution data:

$$g = {β}_{0} + {\sum }_{j = 1}^{C} {β}_{j} {p}_{j} + e , \qquad ( 1 )$$

where g is the expression value for a gene, p is the percentage data determined by the pathologists, and β's are the expression coefficients associated with different cell types. In model (1), C is the number of tissue types under consideration. In the present case, three major tissue types were included, i.e., tumor, stroma, and BPH. βⱼ is the estimate of the relative expression level in cell type j (i.e., the expression coefficient) compared to the overall mean expression level β₀. The regression model was applied to the patient cases in Dataset 1 to obtain the model parameters (β's) and their corresponding p-values, which were used to aid subsequent gene screening. The application to prostate cancer expression data and validation by immunohistochemistry and by correlation of derived βⱼ values with LCM-derived samples assayed by qPCR has been described (Stuart et al., supra).

Identification of stroma-derived genes and development of the diagnostic classifier: It was hypothesized that stroma within and directly adjacent to prostate cancer epithelial cell formations of infiltrating tumors exhibit significant RNA expression changes compared to normal prostate stroma. To obtain an initial comparison of tumor-adjacent stroma to normal stroma, normal fresh frozen biopsy tissue was used as a source of normal stroma. Out of 27 normal biopsy samples, 15 were selected from 15 different participants. The remaining 12 biopsy samples were reserved for testing. Gene expression microarray data were obtained and compared to 13 tumor-bearing patient cases from Dataset 1 selected to tumor (T) greater than 0% but less than 10% tumor cell content (the average stroma content is ˜80%). These criteria ensured that the majority of stroma tissues included were close to tumor, while T<10% ensures that the impact from tumor cells was minimal since the aim was to capture altered expression signals from stroma cells rather than from tumor cells.

As the number of biopsies available was limited, a permutation strategy was adopted to maximize their use. First 13 of the 15 normal biopsy samples were selected and their gene expression was compared to the 13 tumor-adjacent stroma samples using the moderated t-test implemented in the LIMMA package of R (Dalgaard, supra). This comparison yielded 3888 expression changes between these two groups with a p value<0.05.

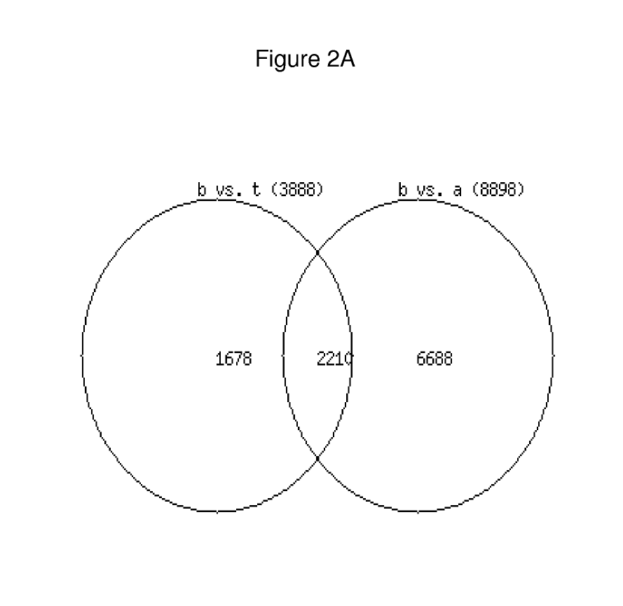

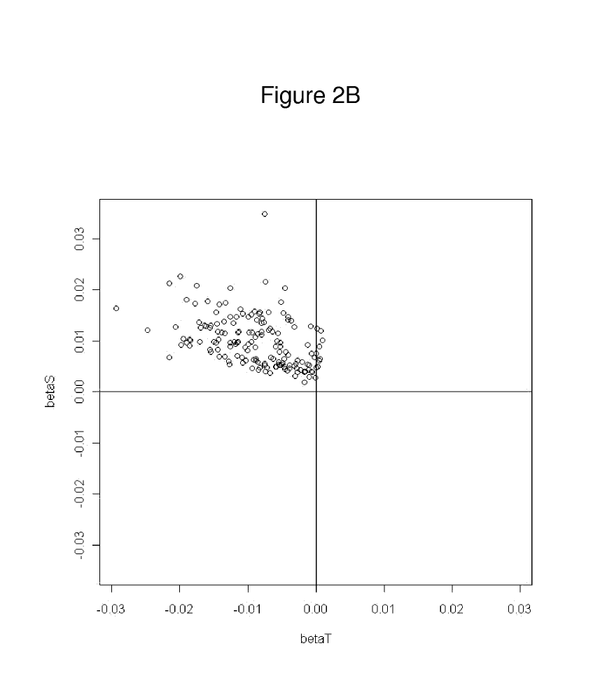

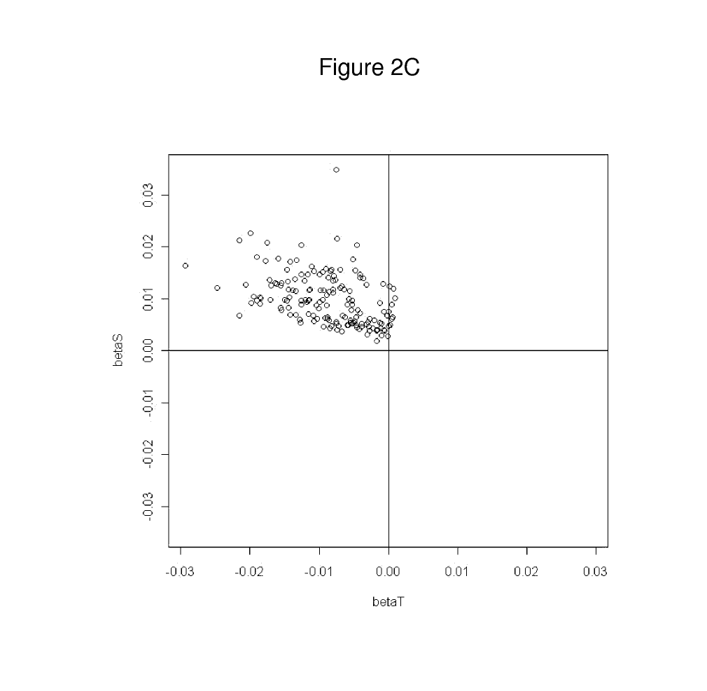

A substantial difference in age existed between the normal stroma group (average age=51.9 years) and the tumor-adjacent stroma group (average age=60.6 years). The overall gene expression of the 13 normal stroma samples used for training was compared to that of 13 normal prostate specimens obtained from the rapid autopsy program (see above), with an average age of 82 years. The comparison revealed 8898 significant expression changes (p<0.05), of which 2210 also were detected in the comparison of normal stroma samples between tumor-adjacent stroma (FIG. 2A). To eliminate potential impact from aging related genes, only 3888−2210=1678 genes were used for further inquiry.

A potential issue related to using patient cases with 10%>T>0% was that the detected expression changes may have included expression changes specific to tumor cells or epithelium cells rather than only to stroma cells. To reduce the possibility that epithelial-cell derived expression changes dominated, a secondary gene screening via MLR analysis was used. MLR was used to determine cell-specific gene expression based on “knowledge” of the percent cell composition of the samples of Dataset 1 as determined by a panel of four pathologists (Stuart et al., supra; the distribution is shown in FIG. 1B for 109 samples from 87 patients of Dataset 1). Thus, the expression data of 109 patient samples was fit with an MLR model by which the comparative signal from individual cell types (i.e., expression coefficients, β's) and corresponding p-values were calculated as described by Stuart et al. (supra). Model diagnostics showed that the fitted model for significant genes (with any significant β's) accounted for >70% of the total variation (or the variation of e in Equation 1 was <30% of the total variation), indicating a plausible modeling scheme. Cell-type specific expression coefficients were then used to identify genes that are largely expressed in stroma by eliminating genes expressed in epithelial cells at greater than 10% of the expression in stroma cells, i.e.,

$${β}_{T} < \frac{1}{10} {β}_{s} .$$

Thus from the 1678 genes of the initial analysis, 160 candidate probe sets with three criteria were selected: (1) γₛ>0, (2) βₛ>10×β_{T}β_{S}>10×β_{T}, and (3) p (βₛ)<0.1. When the values of the βₛ's were compared to the β_{T}'s, it became apparent that the expression levels of these 160 probe sets in stroma cells were substantially higher than in tumor cells (FIG. 2B). Moreover, the average βₛ of these 160 probe sets was 0.011, which was more than two-fold increased compared to the average of any βₛ>0. Thus, the 160 selected probe sets were among the highest expressed stroma genes observed.

The second step for the permutation analysis was then carried out. The above procedure was repeated using a different selections of 13 biopsy samples of the 15 until all 105 possible combinations of 13 normal biopsy samples drawn from 15 (C₁₅¹³=105, where Cₙ^{m} is the number of combinations of m elements chosen from a total of n elements) was complete. A total of 339 probe sets (Table 3) were generated by the 105-fold gene selection procedure with a frequency of selection as summarized in FIG. 1A. Permutation increased the basis set by 339/160, or a 2-fold amplification. Probe sets with at least 50 occurrences (about 50%) of the 105-fold permutation were selected for classifier construction. Prediction Analysis for Microarrays (PAM; Tibshirani et al. (2002) Proc. Natl. Acad. Sci. U.S.A. 99:6567-6572) was used to build a diagnostic classifier. The training set (Table 2, line 1) included all 15 normal biopsies and the 13 tumor-adjacent stroma samples that were used for the derivation of significant differences. Of the 146 PAM-input probe sets, 131 were retained following the 10-fold cross validation procedure of PAM, leading to a prediction accuracy of 96.4%. The separation of normal and tumor-adjacent stroma cases of the training set by the Classifier is illustrated into two distinct populations is shown in FIG. 2C. The complete list of 146 probe-sets, including 131 probe-sets selected by PAM, is given in Table 4. Many of these genes are known by their function and expression in mesenchymal derivatives such as muscle, nerve, and connective tissue.

TABLE 2
Operating characteristics (OC) for training analysis and tests.

| | | | Case | Accura- | Sensitivity | Speci- |
|---|---|---|---|---|---|---|
| | | Dataset | Num. | cy % | % | ficity % |
| 1 | Training set | 1 | 28 | 96.4 | 92.3 | 100 |
| | | | (15 + 13) | | | |
| | Test set | | | | | |
| | Tumor | | | | | |
| 2 | Tumor-bearing | 1 | 55 | 96.4 | 96.4 | NA |
| | | | (68 − 13) | | | |
| 3 | Tumor-bearing | 2 | 65 | 100 | 100 | NA |
| 4 | Tumor-bearing | 3 | 79 | 100 | 100 | NA |
| 5 | Tumor-bearing | 4 | 44 | 100 | 100 | NA |
| | Normals | | | | | |
| 6 | Biopsies (1) | 1 | 7 | 100 | NA | 100 |
| 7 | Biopsies (2) | 1 | 5 | 60 | NA | 60 |
| 8 | Rapid autopsies | 1 | 13 | 92.3 | NA | 92.3 |
| | Manual | | | | | |
| | Microdissected/ | | | | | |
| | LCM | | | | | |
| 9 | Tumor-adjacent | 2 | 71 | 97.1 | 97.1 | NA |
| | Stroma | | | | | |
| 10 | Tumor-adjacent | 4 | 13 | 100 | 100 | NA |
| | Stroma | | | | | |
| 11 | Tumor-adjacent | 1 | 12 | 75 | 75 | NA |
| | Stroma | | | | | |
| 12 | Tumor-bearing | 5 | 12 | 100 | 100 | NA |
| | LCM | | | | | |
| 13 | Normal Stroma | 5 | 4 | 100 | NA | 100 |
| | LCM | | | | | |

Testing with independent datasets: The 131-element classifier was then tested on numerous prostate samples not used for training, including 55 tumor-bearing cases from Dataset 1 and 65 tumor-bearing cases from Dataset 2. Also included were two additional datasets of 79 tumor-bearing cases (Dataset 3) and 44 tumor-bearing cases (Dataset 4), where both the samples and expression analyses were from separate institutes (Table 1). These four test sets were composed entirely of tumor bearing samples (Table 2, lines 2 to 5). In all four tests, almost all samples (n=243) were recognized as “tumor” with high average accuracy ˜99%. FIG. 1B gives the distribution of tumor percentages for the 109 patient cases of Dataset 1. Two misclassified test samples occurred at T=20% and 25% (marked with “*” in FIG. 1B) and therefore are not restricted to the presence of high tumor content. The classification method utilizing PAM did not involve any “knowledge” of cell type content and therefore is successful on samples with a broad range of tumor epithelial cells, including samples with just a low percentage of epithelial cells. Such samples consist of over 90% stroma cells. For the test cases of Dataset 2, tumor cell composition ranges from 2% to 80% (FIG. 1C). For Datasets 3 and 4, the tumor epithelium component was not assessed but was estimated using the CellPred program. This yielded estimates of 24% to over 80% stroma cell content for Dataset 3, and as little as 0% to over 80% stroma cell content for Dataset 4 (FIGS. 1D and 1E). These observations suggested that the classifier is accurate in the classification of independent tumor-bearing samples as “presence of tumor” and does not depend upon “recognition” of gene expression if the tumor epithelial component.

The classifier also was tested using specimens composed mainly of normal prostate stroma and epithelium. First, the classifier was tested on the 12 remaining biopsies from the DMFO study which were separated into two groups. Group 1 (Table 2, line 6) included second biopsies of the same participants whose first biopsy samples were included in the training set, and therefore are not completely independent cases. Group 2 (Table 2, line 7) included the five biopsy samples of cases not used for training. These samples were devoid of tumor but contained normal epithelial components, typically ranging from ˜35% to ˜45%. Microarray data were obtained for these 12 cases and used for testing. The biopsy samples in group 1 were accurately (100%) identified as non-tumor. For group 2, two out of five biopsy samples were categorized as “presence of tumor.” When the histories for these cases were consulted, however, it was found that both had consistently exhibited elevated PSA levels of 6.1, 9.6, and 8 ng/ml (normal values<3 ng/ml), respectively, although no tumor was observed in either of two sets of sextant biopsies obtained from these cases. All other donors of normal biopsies exhibited normal PSA values. The classifier was then tested on 13 specimens obtained by rapid autopsy of individuals dying of unrelated causes (Table 2, line 8). Twelve out of these 13 cases (i.e., 92.3%), were classified as nontumor. Histological examination of all embedded tissue of the two “misclassified” cases revealed multiple foci of small “latent” tumors. The 25 samples which were drawn from normal tissues were correctly classified as having no tumor present, or were classified in accordance with abnormal features that were subsequently uncovered. These results provide further support for the ability of the classifier to discriminate between normal and abnormal prostate tissues in the absence of histologically recognizable tumor cells in the samples studied.

Validation by manual microdissection and LCM of tumor-adjacent and remote stroma: Based on the strong performance with mixed tissue test samples, experiments were conducted to validate the classifier by developing histologically confirmed pure tumor-adjacent stroma samples. Tumor-bearing tissue mounted in OCT blocks in a cryostat were examined by frozen section to visualize the location of the tumor. The OCT-embedded block was etched with a single straight cut with a scalpel to divide the embedded tissue into a tumor zone and tumor-adjacent stroma. Subsequent cryosections were separated into two halves and used for H and E staining to confirm their composition. For sections of tumor-adjacent stroma with a large area (i.e., ˜10 mm²), multiple frozen sections were pooled and used for RNA preparation and microarray hybridization. A final frozen section was stained and examined to confirm that it was free of tumor cells. For smaller areas of the tumor-adjacent zone, the adjacent tissue was removed as a piece, remounted in reverse orientation and a final frozen section was made to confirm that the piece was free of tumor cells. This tissue was then used for RNA preparation and expression analysis.

Seventy-one tumor-adjacent stroma samples were obtained from the samples of Dataset 2, 13 from the samples of Dataset 4, and 12 from the samples of Dataset 1, using the manual microdissection method. These tumor-adjacent stroma samples were then used for expression analysis. The expression values for the 131 classifier probe sets were tested using the PAM procedure. Accuracies of 97.1%, 100%, and 75% were observed for the classification as “presence of tumor” (Table 2, lines 9-11). These results indicate an overall accuracy of 94.7% for the 96 independent samples.

Finally, examined laser capture microdissected samples were prepared from the samples of Dataset 5. Twelve tumor cell samples were prepared as 100% prostate cancer cells, while four pooled stroma control samples were prepared from cases where no tumor had been recovered in the surgical samples available for the research protocol. These samples were categorized by the classifier as 100% “presence of tumor” and 100% “no presence of tumor,” respectively.

Since several cases (especially from Dataset 1) appeared “misclassified,” it was of interest to know how far from a known tumor site the expression changes characteristic of tumor stroma may extend. There was insufficient tissue for a systematic analysis of samples at various known distances, but 28 cases from Dataset 1 were available that were greater than 1.5 cm from the tumor sites of the same gland and generally were from the contralateral lobe of the donor gland. Array data was collected from all pieces and categorized by the classifier. Only ten of the 28 samples (35.7%) were categorized as tumor-associated stroma.

This distribution of classifications was compared to the distribution for the original 12 tumor-adjacent stroma samples manually prepared from samples of Dataset 1 (Table 2, line 11) using the Fisher Exact Test. The distribution for the 28 “remote” samples was significantly different than the category distribution for the 12 authentic tumor-adjacent stroma samples of the same cases as judged by a Fischer Exact test, p=0.038. This result strongly suggests that the expression changes of tumor-adjacent stroma are not inevitable in stroma taken from arbitrary sites of the same tumor-bearing glands, and likely reflect that proximity to tumor affects the expression changes of the genes of the classifier developed here.

Comparison with random-gene classifiers: To further validate the 131-element diagnostic classifier, 100 randomized experiments were carried out. In each experiment, 1,700 probe sets were randomly selected from the 12,901 probe set basis, which was obtained by subtracting 9376 aging related probe sets from the entire 22277 probe sets, where 9376 aging related expression changes were defined exactly as before. Finally, the sampled probe sets were screened with the same MLR criteria used for development of the 131-element classifier, i.e., (1) βₛ>0, (2) βₛ>10×β_{T}, and (3) p (βₛ<0.1). In each random experiment, the genes that survived the MLR filter were used to develop a classifier with PAM exactly as for the 131-probe set classifier. PAM selected an average of 6.2 probe sets (<<131), and the average performance of these random-gene classifiers based on the tests of other datasets are summarized in Table 5. These random-gene classifiers failed to detect the presence of tumor in most of the test sets. The random classifier was particularly poor, however, in defining a normal distribution for Dataset 1, leading an 8.7% (Table 5, line 2) sensitivity suggesting a bias toward “no presence of tumor.” This correlated with the second lack of normal distribution due to a similar bias toward “no presence of tumor,” but this time affecting the normal tissues and thereby giving rise to the appearance of accuracy with an average of 82.3% (Table 5, average lines 6-9 and 13). In general, however, the random model tended to be a normal distribution with poor accuracies in the range of 12.9% to 19.2%, indicating that the results obtained with the developed 131-probe set classifier cannot be attributed to chance.

TABLE 3
Basis set of genes, derived as described herein.

| | | Gene | | | | | |
|---|---|---|---|---|---|---|---|
| Probe Set ID | Gene Title | Symbol | logFC | t | P | Adj. P | B |
| 200067_x_at | sorting nexin 3 | SNX3 | −0.13 | −1.85 | 0.07 | 0.34 | −4.82 |
| 200685_at | splicing factor, | SFRS11 | −0.16 | −2.19 | 0.04 | 0.24 | −4.20 |
| | arginine/serine-rich 11 | | | | | | |
| 200788_s_at | phosphoprotein enriched in | PEA15 | −0.22 | −2.34 | 0.03 | 0.20 | −3.91 |
| | astrocytes 15 | | | | | | |
| 201022_s_at | destrin (actin depolymerizing | DSTN | −0.14 | −2.07 | 0.05 | 0.27 | −4.43 |
| | factor) | | | | | | |
| 201312_s_at | SH3 domain binding glutamic | SH3BGRL | −0.19 | −1.84 | 0.08 | 0.34 | −4.82 |
| | acid-rich protein like | | | | | | |
| 201313_at | enolase 2 (gamma, neuronal) | ENO2 | −0.36 | −2.15 | 0.04 | 0.25 | −4.29 |
| 201344_at | ubiquitin-conjugating enzyme | UBE2D2 | −0.38 | −2.96 | 0.01 | 0.09 | −2.59 |
| | E2D 2 (UBC4/5 homolog, | | | | | | |
| | yeast) | | | | | | |
| 201380_at | cartilage associated protein | CRTAP | −0.22 | −2.00 | 0.05 | 0.29 | −4.56 |
| 201389_at | integrin, alpha 5 (fibronectin | ITGA5 | −0.50 | −2.46 | 0.02 | 0.17 | −3.67 |
| | receptor, alpha polypeptide) | | | | | | |
| 201430_s_at | dihydropyrimidinase-like 3 | DPYSL3 | −0.35 | −1.85 | 0.08 | 0.34 | −4.82 |
| 201431_s_at | dihydropyrimidinase-like 3 | DPYSL3 | −0.40 | −2.78 | 0.01 | 0.12 | −3.00 |
| 201540_at | four and a half LIM domains 1 | FHL1 | −0.23 | −1.94 | 0.06 | 0.31 | −4.66 |
| 201560_at | chloride intracellular channel 4 | CLIC4 | −0.15 | −1.73 | 0.09 | 0.37 | −5.01 |
| 201566_x_at | inhibitor of DNA binding 2, | ID2 | 0.40 | 2.73 | 0.01 | 0.13 | −3.11 |
| | dominant negative helix-loop- | | | | | | |
| | helix protein | | | | | | |
| 201655_s_at | heparan sulfate proteoglycan 2 | HSPG2 | −0.18 | −1.19 | 0.25 | 0.57 | −5.75 |
| 201667_at | gap junction protein, alpha 1, | GJA1 | −0.17 | −1.75 | 0.09 | 0.36 | −4.97 |
| | 43 kDa | | | | | | |
| 201841_s_at | heat shock 27 kDa protein 1 | HSPB1 | −0.44 | −3.97 | 0.00 | 0.02 | −0.12 |
| 201843_s_at | EGF-containing fibulin-like | EFEMP1 | −0.32 | −2.21 | 0.04 | 0.23 | −4.17 |
| | extracellular matrix protein 1 | | | | | | |
| 201980_s_at | Ras suppressor protein 1 | RSU1 | −0.17 | −1.79 | 0.08 | 0.35 | −4.91 |
| 201981_at | pregnancy-associated plasma | PAPPA | −0.24 | −1.51 | 0.14 | 0.45 | −5.34 |
| | protein A, pappalysin 1 | | | | | | |
| 202073_at | optineurin | OPTN | −0.29 | −1.93 | 0.06 | 0.31 | −4.68 |
| 202192_s_at | growth arrest-specific 7 | GAS7 | −0.43 | −1.96 | 0.06 | 0.30 | −4.62 |
| 202196_s_at | dickkopf homolog 3 (Xenopus | DKK3 | −0.15 | −1.29 | 0.21 | 0.53 | −5.63 |
| | laevis) | | | | | | |
| 202202_s_at | laminin, alpha 4 | LAMA4 | −0.35 | −1.83 | 0.08 | 0.34 | −4.85 |
| 202362_at | RAP1A, member of RAS | RAP1A | −0.32 | −1.94 | 0.06 | 0.31 | −4.65 |
| | oncogene family | | | | | | |
| 202422_s_at | acyl-CoA synthetase long- | ACSL4 | −0.16 | −1.08 | 0.29 | 0.62 | −5.87 |
| | chain family member 4 | | | | | | |
| 202432_at | protein phosphatase 3 | PPP3CB | −0.17 | −1.81 | 0.08 | 0.35 | −4.89 |
| | (formerly 2B), catalytic | | | | | | |
| | subunit, beta isoform | | | | | | |
| 202440_s_at | suppression of tumorigenicity 5 | ST5 | −0.17 | −1.26 | 0.22 | 0.54 | −5.66 |
| 202522_at | phosphatidylinositol transfer | PITPNB | −0.16 | −2.85 | 0.01 | 0.11 | −2.85 |
| | protein, beta | | | | | | |
| 202565_s_at | supervillin | SVIL | −0.36 | −2.45 | 0.02 | 0.18 | −3.69 |
| 202588_at | adenylate kinase 1 | AK1 | −0.18 | −1.96 | 0.06 | 0.30 | −4.63 |
| 202613_at | CTP synthase | CTPS | −0.21 | −1.71 | 0.10 | 0.38 | −5.03 |
| 202620_s_at | procollagen-lysine, 2- | PLOD2 | −0.13 | −1.34 | 0.19 | 0.51 | −5.57 |
| | oxoglutarate 5-dioxygenase 2 | | | | | | |
| 202685_s_at | AXL receptor tyrosine kinase | AXL | −0.30 | −1.79 | 0.08 | 0.35 | −4.92 |
| 202796_at | synaptopodin | SYNPO | −0.22 | −1.29 | 0.21 | 0.53 | −5.63 |
| 202806_at | drebrin 1 | DBN1 | −0.43 | −4.08 | 0.00 | 0.02 | 0.17 |
| 202931_x_at | bridging integrator 1 | BIN1 | −0.27 | −2.39 | 0.02 | 0.19 | −3.82 |
| 203151_at | microtubule-associated protein | MAP1A | −0.69 | −4.02 | 0.00 | 0.02 | 0.03 |
| | 1A | | | | | | |
| 203178_at | glycine amidinotransferase (L- | GATM | −0.24 | −1.39 | 0.18 | 0.49 | −5.51 |
| | arginine:glycine | | | | | | |
| | amidinotransferase) | | | | | | |
| 203299_s_at | adaptor-related protein | AP1S2 | −0.41 | −2.77 | 0.01 | 0.12 | −3.01 |
| | complex 1, sigma 2 subunit | | | | | | |
| 203389_at | kinesin family member 3C | KIF3C | −0.26 | −2.39 | 0.02 | 0.19 | −3.82 |
| 203436_at | ribonuclease P/MRP 30 kDa | RPP30 | −0.14 | −1.61 | 0.12 | 0.41 | −5.19 |
| | subunit | | | | | | |
| 203438_at | stanniocalcin 2 | STC2 | −0.37 | −1.80 | 0.08 | 0.35 | −4.90 |
| 203456_at | PRA1 domain family, member 2 | PRAF2 | −0.28 | −2.07 | 0.05 | 0.27 | −4.44 |
| 203501_at | plasma glutamate | PGCP | −0.30 | −2.27 | 0.03 | 0.22 | −4.05 |
| | carboxypeptidase | | | | | | |
| 203597_s_at | WW domain binding protein 4 | WBP4 | −0.34 | −3.56 | 0.00 | 0.04 | −1.17 |
| | (formin binding protein 21) | | | | | | |
| 203705_s_at | frizzled homolog 7 | FZD7 | 0.25 | 1.46 | 0.15 | 0.47 | −5.41 |
| | (Drosophila) | | | | | | |
| 203729_at | epithelial membrane protein 3 | EMP3 | −0.31 | −1.45 | 0.16 | 0.47 | −5.43 |
| 203766_s_at | leiomodin 1 (smooth muscle) | LMOD1 | −0.36 | −2.04 | 0.05 | 0.28 | −4.49 |
| 203939_at | 5′-nucleotidase, ecto (CD73) | NT5E | −0.49 | −3.80 | 0.00 | 0.03 | −0.54 |
| 204030_s_at | schwannomin interacting | SCHIP1 | −0.32 | −1.91 | 0.07 | 0.32 | −4.71 |
| | protein 1 | | | | | | |
| 204036_at | lysophosphatidic acid receptor 1 | LPAR1 | −0.31 | −1.85 | 0.07 | 0.33 | −4.81 |
| 204058_at | malic enzyme 1, NADP(+)- | ME1 | −0.34 | −2.21 | 0.03 | 0.23 | −4.17 |
| | dependent, cytosolic | | | | | | |
| 204059_s_at | malic enzyme 1, NADP(+)- | ME1 | −0.35 | −1.96 | 0.06 | 0.30 | −4.63 |
| | dependent, cytosolic | | | | | | |
| 204115_at | guanine nucleotide binding | GNG11 | −0.22 | −1.34 | 0.19 | 0.51 | −5.57 |
| | protein (G protein), gamma 11 | | | | | | |
| 204134_at | phosphodiesterase 2A, cGMP- | PDE2A | −0.16 | −1.41 | 0.17 | 0.49 | −5.48 |
| | stimulated | | | | | | |
| 204159_at | cyclin-dependent kinase | CDKN2C | −0.46 | −3.42 | 0.00 | 0.05 | −1.49 |
| | inhibitor 2C (p18, inhibits | | | | | | |
| | CDK4) | | | | | | |
| 204302_s_at | KIAA0427 | KIAA0427 | −0.10 | −1.10 | 0.28 | 0.61 | −5.85 |
| 204303_s_at | KIAA0427 | KIAA0427 | −0.35 | −2.17 | 0.04 | 0.24 | −4.25 |
| 204304_s_at | prominin 1 | PROM1 | 0.59 | 1.26 | 0.22 | 0.55 | −5.67 |
| 204365_s_at | receptor accessory protein 1 | REEP1 | −0.29 | −2.18 | 0.04 | 0.24 | −4.23 |
| 204396_s_at | G protein-coupled receptor | GRK5 | −0.46 | −2.09 | 0.05 | 0.27 | −4.40 |
| | kinase 5 | | | | | | |
| 204410_at | eukaryotic translation | EIF1AY | −0.21 | −1.56 | 0.13 | 0.43 | −5.27 |
| | initiation factor 1A, Y-linked | | | | | | |
| 204517_at | peptidylprolyl isomerase C | PPIC | −0.17 | −1.98 | 0.06 | 0.30 | −4.60 |
| | (cyclophilin C) | | | | | | |
| 204557_s_at | DAZ interacting protein 1 | DZIP1 | −0.21 | −1.57 | 0.13 | 0.43 | −5.25 |
| 204570_at | cytochrome c oxidase subunit | COX7A1 | −0.37 | −1.56 | 0.13 | 0.43 | −5.27 |
| | VIIa polypeptide 1 (muscle) | | | | | | |
| 204584_at | L1 cell adhesion molecule | L1CAM | −1.20 | −3.10 | 0.00 | 0.08 | −2.26 |
| 204627_s_at | integrin, beta 3 (platelet | ITGB3 | −0.82 | −3.51 | 0.00 | 0.04 | −1.28 |
| | glycoprotein IIIa, antigen | | | | | | |
| | CD61) | | | | | | |
| 204628_s_at | integrin, beta 3 (platelet | ITGB3 | −0.31 | −2.42 | 0.02 | 0.18 | −3.75 |
| | glycoprotein IIIa, antigen | | | | | | |
| | CD61) | | | | | | |
| 204639_at | adenosine deaminase | ADA | −0.38 | −1.27 | 0.21 | 0.54 | −5.66 |
| 204736_s_at | chondroitin sulfate | CSPG4 | −0.55 | −3.29 | 0.00 | 0.06 | −1.81 |
| | proteoglycan 4 | | | | | | |
| 204777_s_at | mal, T-cell differentiation | MAL | −0.99 | −3.32 | 0.00 | 0.06 | −1.74 |
| | protein | | | | | | |
| 204939_s_at | phospholamban | PLN | −0.45 | −2.53 | 0.02 | 0.16 | −3.53 |
| 204940_at | phospholamban | PLN | −0.49 | −2.45 | 0.02 | 0.18 | −3.70 |
| 204963_at | sarcospan (Kras oncogene- | SSPN | −0.26 | −1.97 | 0.06 | 0.30 | −4.61 |
| | associated gene) | | | | | | |
| 205076_s_at | myotubularin related protein | MTMR11 | −0.57 | −2.92 | 0.01 | 0.10 | −2.69 |
| | 11 | | | | | | |
| 205111_s_at | phospholipase C, epsilon 1 | PLCE1 | −0.35 | −1.53 | 0.14 | 0.44 | −5.30 |
| 205132_at | actin, alpha, cardiac muscle 1 | ACTC1 | −0.99 | −3.28 | 0.00 | 0.06 | −1.83 |
| 205231_s_at | epilepsy, progressive | EPM2A | −0.42 | −2.97 | 0.01 | 0.09 | −2.56 |
| | myoclonus type 2A, Lafora | | | | | | |
| | disease (laforin) | | | | | | |
| 205257_s_at | amphiphysin | AMPH | −0.22 | −1.75 | 0.09 | 0.37 | −4.98 |
| 205265_s_at | SPEG complex locus | SPEG | −0.31 | −1.68 | 0.10 | 0.39 | −5.09 |
| 205303_at | potassium inwardly-rectifying | KCNJ8 | −0.42 | −2.88 | 0.01 | 0.10 | −2.77 |
| | channel, subfamily J, member 8 | | | | | | |
| 205304_s_at | potassium inwardly-rectifying | KCNJ8 | −0.24 | −1.83 | 0.08 | 0.34 | −4.84 |
| | channel, subfamily J, member 8 | | | | | | |
| 205325_at | phytanoyl-CoA 2-hydroxylase | PHYHIP | −0.42 | −1.49 | 0.15 | 0.46 | −5.37 |
| | interacting protein | | | | | | |
| 205368_at | family with sequence | FAM131B | −0.27 | −2.31 | 0.03 | 0.21 | −3.98 |
| | similarity 131, member B | | | | | | |
| 205384_at | FXYD domain containing ion | FXYD1 | −0.52 | −1.81 | 0.08 | 0.34 | −4.87 |
| | transport regulator 1 | | | | | | |
| | (phospholemman) | | | | | | |
| 205398_s_at | SMAD family member 3 | SMAD3 | −0.22 | −1.52 | 0.14 | 0.45 | −5.33 |
| 205433_at | butyrylcholinesterase | BCHE | −0.93 | −2.52 | 0.02 | 0.16 | −3.55 |
| 205475_at | scrapie responsive protein 1 | SCRG1 | −0.45 | −1.87 | 0.07 | 0.33 | −4.78 |
| 205478_at | protein phosphatase 1, | PPP1R1A | −0.36 | −1.58 | 0.12 | 0.43 | −5.24 |
| | regulatory (inhibitor) subunit | | | | | | |
| | 1A | | | | | | |
| 205554_s_at | deoxyribonuclease I-like 3 | DNASE1L3 | 0.35 | 1.57 | 0.13 | 0.43 | −5.25 |
| 205561_at | potassium channel | KCTD17 | −0.32 | −2.77 | 0.01 | 0.12 | −3.02 |
| | tetramerisation domain | | | | | | |
| | containing 17 | | | | | | |
| 205611_at | tumor necrosis factor (ligand) | TNFSF12 | −0.29 | −2.18 | 0.04 | 0.24 | −4.22 |
| | superfamily, member 12 | | | | | | |
| 205618_at | proline rich Gla (G- | PRRG1 | −0.16 | −1.26 | 0.22 | 0.54 | −5.66 |
| | carboxyglutamic acid) 1 | | | | | | |
| 205632_s_at | phosphatidylinositol-4- | PIP5K1B | −0.43 | −1.96 | 0.06 | 0.30 | −4.63 |
| | phosphate 5-kinase, type I, | | | | | | |
| | beta | | | | | | |
| 205674_x_at | FXYD domain containing ion | FXYD2 | −0.14 | −1.10 | 0.28 | 0.61 | −5.85 |
| | transport regulator 2 | | | | | | |
| 205792_at | WNT1 inducible signaling | WISP2 | −0.66 | −1.89 | 0.07 | 0.32 | −4.74 |
| | pathway protein 2 | | | | | | |
| 205954_at | retinoid X receptor, gamma | RXRG | −0.53 | −3.47 | 0.00 | 0.04 | −1.38 |
| 205973_at | fasciculation and elongation | FEZ1 | −0.35 | −2.38 | 0.02 | 0.19 | −3.83 |
| | protein zeta 1 (zygin I) | | | | | | |
| 206024_at | 4-hydroxyphenylpyruvate | HPD | −0.57 | −2.79 | 0.01 | 0.12 | −2.98 |
| | dioxygenase | | | | | | |
| 206132_at | mutated in colorectal cancers | MCC | 0.48 | 2.01 | 0.05 | 0.29 | −4.53 |
| 206201_s_at | mesenchyme homeobox 2 | MEOX2 | −0.53 | −1.65 | 0.11 | 0.40 | −5.13 |
| 206283_s_at | T-cell acute lymphocytic | TAL1 | −0.26 | −1.93 | 0.06 | 0.31 | −4.68 |
| | leukemia 1 | | | | | | |
| 206289_at | homeobox A4 | HOXA4 | −0.29 | −2.36 | 0.03 | 0.20 | −3.88 |
| 206306_at | ryanodine receptor 3 | RYR3 | −0.46 | −1.85 | 0.07 | 0.33 | −4.81 |
| 206331_at | calcitonin receptor-like | CALCRL | −0.27 | −1.80 | 0.08 | 0.35 | −4.90 |
| 206382_s_at | brain-derived neurotrophic | BDNF | −0.62 | −2.89 | 0.01 | 0.10 | −2.74 |
| | factor | | | | | | |
| 206423_at | angiopoietin-like 7 | ANGPTL7 | −0.47 | −1.94 | 0.06 | 0.31 | −4.66 |
| 206425_s_at | transient receptor potential | TRPC3 | −0.57 | −3.31 | 0.00 | 0.06 | −1.77 |
| | cation channel, subfamily C, | | | | | | |
| | member 3 | | | | | | |
| 206510_at | SIX homeobox 2 | SIX2 | −0.60 | −1.61 | 0.12 | 0.42 | −5.19 |
| 206525_at | gamma-aminobutyric acid | GABRR1 | 0.15 | 1.07 | 0.29 | 0.62 | −5.88 |
| | (GABA) receptor, rho 1 | | | | | | |
| 206560_s_at | melanoma inhibitory activity | MIA | −0.19 | −1.72 | 0.10 | 0.38 | −5.03 |
| 206580_s_at | EGF-containing fibulin-like | EFEMP2 | −0.21 | −1.29 | 0.21 | 0.53 | −5.63 |
| | extracellular matrix protein 2 | | | | | | |
| 206874_s_at | — | — | −0.44 | −4.27 | 0.00 | 0.01 | 0.66 |
| 206898_at | cadherin 19, type 2 | CDH19 | −0.48 | −2.00 | 0.05 | 0.29 | −4.56 |
| 207071_s_at | aconitase 1, soluble | ACO1 | −0.27 | −2.90 | 0.01 | 0.10 | −2.72 |
| 207303_at | phosphodiesterase 1C, | PDE1C | −0.24 | −1.74 | 0.09 | 0.37 | −5.00 |
| | calmodulin-dependent 70 kDa | | | | | | |
| 207332_s_at | transferrin receptor (p90, | TFRC | 0.18 | 1.32 | 0.20 | 0.52 | −5.59 |
| | CD71) | | | | | | |
| 207437_at | neuro-oncological ventral | NOVA1 | −0.43 | −1.58 | 0.13 | 0.43 | −5.24 |
| | antigen 1 | | | | | | |
| 207554_x_at | thromboxane A2 receptor | TBXA2R | −0.44 | −2.86 | 0.01 | 0.11 | −2.82 |
| 207834_at | fibulin 1 | FBLN1 | −0.35 | −1.98 | 0.06 | 0.30 | −4.59 |
| 207876_s_at | filamin C, gamma (actin | FLNC | −0.45 | −2.98 | 0.01 | 0.09 | −2.55 |
| | binding protein 280) | | | | | | |
| 208131_s_at | prostaglandin I2 (prostacyclin) | PTGIS | −0.28 | −2.02 | 0.05 | 0.28 | −4.51 |
| | synthase | | | | | | |
| 208760_at | Ubiquitin-conjugating enzyme | UBE2I | −0.24 | −1.84 | 0.08 | 0.34 | −4.83 |
| | E2I (UBC9 homolog, yeast) | | | | | | |
| 208789_at | polymerase I and transcript | PTRF | −0.42 | −2.27 | 0.03 | 0.22 | −4.06 |
| | release factor | | | | | | |
| 208792_s_at | clusterin | CLU | −0.15 | −1.03 | 0.31 | 0.64 | −5.92 |
| 208869_s_at | GABA(A) receptor-associated | GABARAPL1 | −0.19 | −2.73 | 0.01 | 0.13 | −3.11 |
| | protein like 1 | | | | | | |
| 209015_s_at | DnaJ (Hsp40) homolog, | DNAJB6 | −0.29 | −2.61 | 0.01 | 0.15 | −3.36 |
| | subfamily B, member 6 | | | | | | |
| 209086_x_at | melanoma cell adhesion | MCAM | −0.61 | −4.06 | 0.00 | 0.02 | 0.12 |
| | molecule | | | | | | |
| 209087_x_at | melanoma cell adhesion | MCAM | −0.40 | −2.32 | 0.03 | 0.21 | −3.96 |
| | molecule | | | | | | |
| 209167_at | glycoprotein M6B | GPM6B | −0.22 | −2.14 | 0.04 | 0.25 | −4.30 |
| 209168_at | glycoprotein M6B | GPM6B | −0.18 | −1.59 | 0.12 | 0.42 | −5.22 |
| 209169_at | glycoprotein M6B | GPM6B | −0.34 | −3.16 | 0.00 | 0.07 | −2.13 |
| 209170_s_at | glycoprotein M6B | GPM6B | −0.23 | −1.61 | 0.12 | 0.41 | −5.19 |
| 209191_at | tubulin, beta 6 | TUBB6 | −0.51 | −2.92 | 0.01 | 0.10 | −2.67 |
| 209242_at | paternally expressed 3 | PEG3 | −0.25 | −1.64 | 0.11 | 0.41 | −5.15 |
| 209263_x_at | tetraspanin 4 | TSPAN4 | −0.17 | −1.42 | 0.17 | 0.48 | −5.46 |
| 209288_s_at | CDC42 effector protein (Rho | CDC42EP3 | −0.21 | −1.86 | 0.07 | 0.33 | −4.79 |
| | GTPase binding) 3 | | | | | | |
| 209293_x_at | inhibitor of DNA binding 4, | ID4 | 0.18 | 1.60 | 0.12 | 0.42 | −5.21 |
| | dominant negative helix-loop- | | | | | | |
| | helix protein | | | | | | |
| 209298_s_at | intersectin 1 (SH3 domain | ITSN1 | −0.21 | −1.66 | 0.11 | 0.40 | −5.12 |
| | protein) | | | | | | |
| 209356_x_at | EGF-containing fibulin-like | EFEMP2 | −0.23 | −1.49 | 0.15 | 0.46 | −5.36 |
| | extracellular matrix protein 2 | | | | | | |
| 209362_at | mediator complex subunit 21 | MED21 | −0.26 | −2.58 | 0.02 | 0.15 | −3.43 |
| 209454_s_at | TEA domain family member 3 | TEAD3 | −0.23 | −1.71 | 0.10 | 0.38 | −5.04 |
| 209488_s_at | RNA binding protein with | RBPMS | −0.33 | −1.83 | 0.08 | 0.34 | −4.84 |
| | multiple splicing | | | | | | |
| 209524_at | hepatoma-derived growth | HDGFRP3 | −0.14 | −2.18 | 0.04 | 0.24 | −4.22 |
| | factor, related protein 3 | | | | | | |
| 209543_s_at | CD34 molecule | CD34 | −0.15 | −1.58 | 0.12 | 0.42 | −5.23 |
| 209612_s_at | alcohol dehydrogenase 1B | ADH1B | −0.41 | −1.20 | 0.24 | 0.57 | −5.74 |
| | (class I), beta polypeptide | | | | | | |
| 209613_s_at | alcohol dehydrogenase 1B | ADH1B | −0.63 | −1.96 | 0.06 | 0.30 | −4.63 |
| | (class I), beta polypeptide | | | | | | |
| 209614_at | alcohol dehydrogenase 1B | ADH1B | −0.24 | −1.89 | 0.07 | 0.32 | −4.75 |
| | (class I), beta polypeptide | | | | | | |
| 209651_at | transforming growth factor | TGFB1I1 | −0.42 | −2.62 | 0.01 | 0.14 | −3.35 |
| | beta 1 induced transcript 1 | | | | | | |
| 209685_s_at | protein kinase C, beta 1 | PRKCB1 | −0.26 | −1.29 | 0.21 | 0.53 | −5.63 |
| 209686_at | S100 calcium binding protein B | S100B | −0.94 | −3.82 | 0.00 | 0.03 | −0.50 |
| 209758_s_at | microfibrillar associated | MFAP5 | −1.48 | −7.89 | 0.00 | 0.00 | 10.08 |
| | protein 5 | | | | | | |
| 209764_at | mannosyl (beta-1,4-)- | MGAT3 | −0.17 | −1.65 | 0.11 | 0.40 | −5.14 |
| | glycoprotein beta-1,4-N- | | | | | | |
| | acetylglucosaminyltransferase | | | | | | |
| 209765_at | ADAM metallopeptidase | ADAM19 | −0.36 | −1.78 | 0.09 | 0.36 | −4.93 |
| | domain 19 (meltrin beta) | | | | | | |
| 209843_s_at | SRY (sex determining region | SOX10 | −0.61 | −5.58 | 0.00 | 0.00 | 4.16 |
| | Y)-box 10 | | | | | | |
| 209859_at | tripartite motif-containing 9 | TRIM9 | −0.19 | −1.09 | 0.28 | 0.61 | −5.85 |
| 209915_s_at | neurexin 1 | NRXN1 | −0.80 | −4.05 | 0.00 | 0.02 | 0.08 |
| 209981_at | cold shock domain containing | CSDC2 | −0.56 | −2.43 | 0.02 | 0.18 | −3.73 |
| | C2, RNA binding | | | | | | |
| 210198_s_at | proteolipid protein 1 | PLP1 | −1.18 | −4.91 | 0.00 | 0.00 | 2.36 |
| | (Pelizaeus-Merzbacher | | | | | | |
| | disease, spastic paraplegia 2, | | | | | | |
| | uncomplicated) | | | | | | |
| 210201_x_at | bridging integrator 1 | BIN1 | −0.29 | −2.54 | 0.02 | 0.16 | −3.52 |
| 210270_at | regulator of G-protein | RGS6 | −0.17 | −1.55 | 0.13 | 0.43 | −5.28 |
| | signaling 6 | | | | | | |
| 210277_at | adaptor-related protein | AP4S1 | −0.22 | −1.34 | 0.19 | 0.51 | −5.57 |
| | complex 4, sigma 1 subunit | | | | | | |
| 210280_at | myelin protein zero (Charcot- | MPZ | −1.20 | −5.02 | 0.00 | 0.00 | 2.64 |
| | Marie-Tooth neuropathy 1B) | | | | | | |
| 210319_x_at | msh homeobox 2 | MSX2 | 0.45 | 2.31 | 0.03 | 0.21 | −3.98 |
| 210432_s_at | sodium channel, voltage-gated, | SCN3A | −0.46 | −1.94 | 0.06 | 0.31 | −4.66 |
| | type III, alpha subunit | | | | | | |
| 210632_s_at | sarcoglycan, alpha (50 kDa | SGCA | −0.58 | −2.55 | 0.02 | 0.16 | −3.49 |
| | dystrophin-associated | | | | | | |
| | glycoprotein) | | | | | | |
| 210736_x_at | dystrobrevin, alpha | DTNA | −0.22 | −1.59 | 0.12 | 0.42 | −5.23 |
| 210814_at | transient receptor potential | TRPC3 | −0.75 | −3.30 | 0.00 | 0.06 | −1.80 |
| | cation channel, subfamily C, | | | | | | |
| | member 3 | | | | | | |
| 210852_s_at | aminoadipate-semialdehyde | AASS | 0.24 | 2.06 | 0.05 | 0.27 | −4.46 |
| | synthase | | | | | | |
| 210869_s_at | melanoma cell adhesion | MCAM | −0.71 | −3.93 | 0.00 | 0.02 | −0.21 |
| | molecule | | | | | | |
| 210872_x_at | growth arrest-specific 7 | GAS7 | −0.17 | −1.32 | 0.20 | 0.52 | −5.59 |
| 210941_at | protocadherin 7 | PCDH7 | 0.31 | 2.05 | 0.05 | 0.28 | −4.46 |
| 211006_s_at | potassium voltage-gated | KCNB1 | −0.31 | −1.89 | 0.07 | 0.32 | −4.75 |
| | channel, Shab-related | | | | | | |
| | subfamily, member 1 | | | | | | |
| 211275_s_at | glycogenin 1 | GYG1 | −0.20 | −1.66 | 0.11 | 0.40 | −5.12 |
| 211276_at | transcription elongation factor | TCEAL2 | −0.52 | −2.89 | 0.01 | 0.10 | −2.75 |
| | A (SII)-like 2 | | | | | | |
| 211340_s_at | melanoma cell adhesion | MCAM | −0.46 | −3.05 | 0.00 | 0.08 | −2.38 |
| | molecule | | | | | | |
| 211347_at | CDC14 cell division cycle 14 | CDC14B | −0.21 | −2.21 | 0.03 | 0.23 | −4.16 |
| | homolog B (S. cerevisiae) | | | | | | |
| 211348_s_at | CDC14 cell division cycle 14 | CDC14B | −0.17 | −1.72 | 0.10 | 0.38 | −5.02 |
| | homolog B (S. cerevisiae) | | | | | | |
| 211491_at | adrenergic, alpha-1A-, | ADRA1A | −0.28 | −1.80 | 0.08 | 0.35 | −4.90 |
| | receptor | | | | | | |
| 211562_s_at | leiomodin 1 (smooth muscle) | LMOD1 | −0.39 | −1.67 | 0.11 | 0.39 | −5.10 |
| 211564_s_at | PDZ and LIM domain 4 | PDLIM4 | −0.16 | −1.05 | 0.30 | 0.63 | −5.90 |
| 211673_s_at | molybdenum cofactor | MOCS1 | −0.19 | −1.23 | 0.23 | 0.55 | −5.70 |
| | synthesis 1 | | | | | | |
| 211677_x_at | cell adhesion molecule 3 | CADM3 | −0.21 | −2.08 | 0.05 | 0.27 | −4.41 |
| 211717_at | ankyrin repeat domain 40 | ANKRD40 | −0.28 | −2.76 | 0.01 | 0.12 | −3.03 |
| 211954_s_at | importin 5 | IPO5 | −0.15 | −2.05 | 0.05 | 0.28 | −4.46 |
| 211964_at | collagen, type IV, alpha 2 | COL4A2 | −0.39 | −2.27 | 0.03 | 0.22 | −4.06 |
| 212086_x_at | lamin A/C | LMNA | 0.25 | 1.74 | 0.09 | 0.37 | −5.00 |
| 212097_at | caveolin 1, caveolae protein, | CAV1 | −0.38 | −4.57 | 0.00 | 0.01 | 1.46 |
| | 22 kDa | | | | | | |
| 212119_at | ras homolog gene family, | RHOQ | −0.18 | −2.08 | 0.05 | 0.27 | −4.42 |
| | member Q | | | | | | |
| 212120_at | ras homolog gene family, | RHOQ | −0.31 | −2.60 | 0.01 | 0.15 | −3.39 |
| | member Q | | | | | | |
| 212274_at | lipin 1 | LPIN1 | −0.48 | −3.92 | 0.00 | 0.02 | −0.25 |
| 212358_at | CAP-GLY domain containing | CLIP3 | −0.47 | −2.34 | 0.03 | 0.20 | −3.92 |
| | linker protein 3 | | | | | | |
| 212385_at | transcription factor 4 | TCF4 | 0.30 | 2.07 | 0.05 | 0.27 | −4.43 |
| 212457_at | transcription factor binding to | TFE3 | −0.25 | −2.38 | 0.02 | 0.19 | −3.84 |
| | IGHM enhancer 3 | | | | | | |
| 212509_s_at | matrix-remodelling associated 7 | MXRA7 | −0.27 | −2.66 | 0.01 | 0.14 | −3.26 |
| 212526_at | spastic paraplegia 20 (Troyer | SPG20 | −0.17 | −1.91 | 0.07 | 0.32 | −4.71 |
| | syndrome) | | | | | | |
| 212565_at | serine/threonine kinase 38 like | STK38L | −0.58 | −3.83 | 0.00 | 0.03 | −0.47 |
| 212589_at | related RAS viral (r-ras) | RRAS2 | −0.29 | −2.84 | 0.01 | 0.11 | −2.86 |
| | oncogene homolog 2 | | | | | | |
| 212610_at | protein tyrosine phosphatase, | PTPN11 | −0.23 | −2.24 | 0.03 | 0.22 | −4.12 |
| | non-receptor type 11 (Noonan | | | | | | |
| | syndrome 1) | | | | | | |
| 212647_at | related RAS viral (r-ras) | RRAS | −0.39 | −1.71 | 0.10 | 0.38 | −5.05 |
| | oncogene homolog | | | | | | |
| 212707_s_at | RAS p21 protein activator 4 /// | FLJ21767 | −0.20 | −1.40 | 0.17 | 0.49 | −5.49 |
| | hypothetical protein FLJ21767 | /// | | | | | |
| | /// similar to HSPC047 protein | LOC100132214 | | | | | |
| | /// similar to RAS p21 protein | /// | | | | | |
| | activator 4 | LOC100133005 | | | | | |
| | | /// | | | | | |
| | | RASA4 | | | | | |
| 212747_at | ankyrin repeat and sterile | ANKS1A | −0.17 | −1.41 | 0.17 | 0.49 | −5.48 |
| | alpha motif domain containing | | | | | | |
| | 1A | | | | | | |
| 212764_at | zinc finger E-box binding | ZEB1 | −0.24 | −1.79 | 0.08 | 0.35 | −4.92 |
| | homeobox 1 | | | | | | |
| 212793_at | dishevelled associated | DAAM2 | −0.56 | −3.95 | 0.00 | 0.02 | −0.17 |
| | activator of morphogenesis 2 | | | | | | |
| 212848_s_at | chromosome 9 open reading | C9orf3 | −0.27 | −2.22 | 0.03 | 0.23 | −4.16 |
| | frame 3 | | | | | | |
| 212886_at | coiled-coil domain containing | CCDC69 | −0.59 | −3.96 | 0.00 | 0.02 | −0.13 |
| | 69 | | | | | | |
| 212887_at | Sec23 homolog A (S. cerevisiae) | SEC23A | −0.20 | −1.86 | 0.07 | 0.33 | −4.79 |
| 212992_at | AHNAK nucleoprotein 2 | AHNAK2 | −0.60 | −2.71 | 0.01 | 0.13 | −3.14 |
| 213010_at | protein kinase C, delta binding | PRKCDBP | −0.47 | −1.99 | 0.06 | 0.29 | −4.57 |
| | protein | | | | | | |
| 213107_at | TRAF2 and NCK interacting | TNIK | 0.40 | 2.03 | 0.05 | 0.28 | −4.49 |
| | kinase | | | | | | |
| 213181_s_at | molybdenum cofactor | MOCS1 | −0.21 | −1.57 | 0.13 | 0.43 | −5.25 |
| | synthesis 1 | | | | | | |
| 213203_at | small nuclear RNA activating | SNAPC5 | −0.15 | −1.56 | 0.13 | 0.43 | −5.27 |
| | complex, polypeptide 5, | | | | | | |
| | 19 kDa | | | | | | |
| 213231_at | dystrophia myotonica, WD | DMWD | −0.30 | −2.40 | 0.02 | 0.19 | −3.79 |
| | repeat containing | | | | | | |
| 213274_s_at | cathepsin B | CTSB | −0.30 | −1.53 | 0.14 | 0.44 | −5.32 |
| 213428_s_at | collagen, type VI, alpha 1 | COL6A1 | −0.21 | −1.37 | 0.18 | 0.50 | −5.52 |
| 213480_at | vesicle-associated membrane | VAMP4 | −0.24 | −2.61 | 0.01 | 0.15 | −3.36 |
| | protein 4 | | | | | | |
| 213545_x_at | sorting nexin 3 | SNX3 | −0.11 | −1.41 | 0.17 | 0.49 | −5.48 |
| 213547_at | cullin-associated and | CAND2 | −0.31 | −2.41 | 0.02 | 0.18 | −3.77 |
| | neddylation-dissociated 2 | | | | | | |
| | (putative) | | | | | | |
| 213630_at | NAC alpha domain containing | NACAD | −0.18 | −1.42 | 0.16 | 0.48 | −5.46 |
| 213675_at | CDNA FLJ25106 fis, clone | — | −0.44 | −3.25 | 0.00 | 0.06 | −1.92 |
| | CBR01467 | | | | | | |
| 213764_s_at | microfibrillar associated | MFAP5 | −1.73 | −7.18 | 0.00 | 0.00 | 8.33 |
| | protein 5 | | | | | | |
| 213765_at | microfibrillar associated | MFAP5 | −1.36 | −6.40 | 0.00 | 0.00 | 6.31 |
| | protein 5 | | | | | | |
| 213808_at | Clone 23688 mRNA sequence | — | −0.43 | −2.16 | 0.04 | 0.25 | −4.26 |
| 213847_at | peripherin | PRPH | −0.93 | −4.12 | 0.00 | 0.02 | 0.27 |
| 213924_at | Metallophosphoesterase 1 | MPPE1 | −0.26 | −1.72 | 0.10 | 0.38 | −5.02 |
| 214023_x_at | tubulin, beta 2B | TUBB2B | −0.75 | −4.21 | 0.00 | 0.01 | 0.51 |
| 214027_x_at | desmin /// family with | DES /// | −0.42 | −1.97 | 0.06 | 0.30 | −4.61 |
| | sequence similarity 48, | FAM48A | | | | | |
| | member A | | | | | | |
| 214039_s_at | lysosomal associated protein | LAPTM4B | −0.17 | −1.20 | 0.24 | 0.57 | −5.73 |
| | transmembrane 4 beta | | | | | | |
| 214078_at | Primary neuroblastoma cDNA, | — | −0.35 | −1.44 | 0.16 | 0.47 | −5.43 |
| | clone:Nbla04246, full insert | | | | | | |
| | sequence | | | | | | |
| 214121_x_at | PDZ and LIM domain 7 | PDLIM7 | −0.32 | −1.68 | 0.10 | 0.39 | −5.08 |
| | (enigma) | | | | | | |
| 214122_at | PDZ and LIM domain 7 | PDLIM7 | −0.30 | −2.74 | 0.01 | 0.13 | −3.09 |
| | (enigma) | | | | | | |
| 214159_at | Phospholipase C, epsilon 1 | PLCE1 | −0.27 | −1.79 | 0.08 | 0.35 | −4.91 |
| 214174_s_at | PDZ and LIM domain 4 | PDLIM4 | −0.23 | −1.43 | 0.16 | 0.48 | −5.45 |
| 214175_x_at | PDZ and LIM domain 4 | PDLIM4 | −0.27 | −1.54 | 0.14 | 0.44 | −5.30 |
| 214212_x_at | fermitin family homolog 2 | FERMT2 | −0.42 | −3.00 | 0.01 | 0.09 | −2.50 |
| | (Drosophila) | | | | | | |
| 214247_s_at | dickkopf homolog 3 (Xenopus | DKK3 | −0.17 | −1.51 | 0.14 | 0.45 | −5.34 |
| | laevis) | | | | | | |
| 214297_at | chondroitin sulfate | CSPG4 | −0.45 | −1.78 | 0.09 | 0.36 | −4.94 |
| | proteoglycan 4 | | | | | | |
| 214306_at | optic atrophy 1 (autosomal | OPA1 | −0.27 | −2.67 | 0.01 | 0.14 | −3.23 |
| | dominant) | | | | | | |
| 214368_at | RAS guanyl releasing protein | RASGRP2 | −0.23 | −2.08 | 0.05 | 0.27 | −4.40 |
| | 2 (calcium and DAG- | | | | | | |
| | regulated) | | | | | | |
| 214434_at | heat shock 70 kDa protein 12A | HSPA12A | −0.57 | −3.40 | 0.00 | 0.05 | −1.54 |
| 214439_x_at | bridging integrator 1 | BIN1 | −0.29 | −2.56 | 0.02 | 0.16 | −3.47 |
| 214449_s_at | ras homolog gene family, | RHOQ | −0.18 | −1.81 | 0.08 | 0.34 | −4.88 |
| | member Q | | | | | | |
| 214600_at | TEA domain family member 1 | TEAD1 | −0.28 | −1.61 | 0.12 | 0.42 | −5.19 |
| | (SV40 transcriptional enhancer | | | | | | |
| | factor) | | | | | | |
| 214606_at | tetraspanin 2 | TSPAN2 | −0.54 | −4.01 | 0.00 | 0.02 | −0.02 |
| 214643_x_at | bridging integrator 1 | BIN1 | −0.23 | −2.16 | 0.04 | 0.25 | −4.27 |
| 214696_at | chromosome 17 open reading | C17orf91 | 0.50 | 1.92 | 0.07 | 0.31 | −4.70 |
| | frame 91 | | | | | | |
| 214767_s_at | heat shock protein, alpha- | HSPB6 | −0.88 | −4.27 | 0.00 | 0.01 | 0.66 |
| | crystallin-related, B6 | | | | | | |
| 214954_at | sushi domain containing 5 | SUSD5 | −0.98 | −3.42 | 0.00 | 0.05 | −1.51 |
| 214987_at | CDNA clone | — | −0.29 | −1.94 | 0.06 | 0.31 | −4.66 |
| | IMAGE: 4801326 | | | | | | |
| 215000_s_at | fasciculation and elongation | FEZ2 | −0.14 | −1.99 | 0.06 | 0.29 | −4.57 |
| | protein zeta 2 (zygin II) | | | | | | |
| 215104_at | nuclear receptor interacting | NRIP2 | −0.94 | −4.62 | 0.00 | 0.01 | 1.59 |
| | protein 2 | | | | | | |
| 215306_at | MRNA; cDNA | — | −0.48 | −2.66 | 0.01 | 0.14 | −3.26 |
| | DKFZp586N2020 (from clone | | | | | | |
| | DKFZp586N2020) | | | | | | |
| 215534_at | MRNA; cDNA | — | −0.46 | −2.46 | 0.02 | 0.17 | −3.68 |
| | DKFZp586C1923 (from clone | | | | | | |
| | DKFZp586C1923) | | | | | | |
| 216096_s_at | neurexin 1 | NRXN1 | −0.37 | −1.68 | 0.10 | 0.39 | −5.08 |
| 216500_at | HL14 gene encoding beta- | — | −0.29 | −2.31 | 0.03 | 0.21 | −3.98 |
| | galactoside-binding lectin, 3′ | | | | | | |
| | end, clone 2 | | | | | | |
| 216894_x_at | cyclin-dependent kinase | CDKN1C | −0.27 | −2.45 | 0.02 | 0.18 | −3.69 |
| | inhibitor 1C (p57, Kip2) | | | | | | |
| 217066_s_at | dystrophia myotonica-protein | DMPK | −0.29 | −2.11 | 0.04 | 0.26 | −4.37 |
| | kinase | | | | | | |
| 217589_at | RAB40A, member RAS | RAB40A | 0.37 | 1.49 | 0.15 | 0.46 | −5.36 |
| | oncogene family | | | | | | |
| 217764_s_at | RAB31, member RAS | RAB31 | −0.21 | −1.38 | 0.18 | 0.50 | −5.51 |
| | oncogene family | | | | | | |
| 217820_s_at | enabled homolog (Drosophila) | ENAH | −0.19 | −2.12 | 0.04 | 0.26 | −4.33 |
| 217880_at | cell division cycle 27 homolog | CDC27 | −0.16 | −1.54 | 0.13 | 0.44 | −5.30 |
| | (S. cerevisiae) | | | | | | |
| 218087_s_at | sorbin and SH3 domain | SORBS1 | −0.18 | −2.00 | 0.05 | 0.29 | −4.56 |
| | containing 1 | | | | | | |
| 218094_s_at | dysbindin (dystrobrevin | DBNDD2 | −0.41 | −3.66 | 0.00 | 0.03 | −0.90 |
| | binding protein 1) domain | /// SYS1- | | | | | |
| | containing 2 /// SYS1- | DBNDD2 | | | | | |
| | DBNDD2 | | | | | | |
| 218183_at | chromosome 16 open reading | C16orf5 | −0.16 | −1.63 | 0.11 | 0.41 | −5.16 |
| | frame 5 | | | | | | |
| 218204_s_at | FYVE and coiled-coil domain | FYCO1 | −0.16 | −1.57 | 0.13 | 0.43 | −5.25 |
| | containing 1 | | | | | | |
| 218208_at | PQ loop repeat containing 1 /// | LOC100131178 | −0.23 | −1.79 | 0.08 | 0.35 | −4.91 |
| | hypothetical protein | /// | | | | | |
| | LOC100131178 | PQLC1 | | | | | |
| 218266_s_at | frequenin homolog | FREQ | −0.46 | −2.32 | 0.03 | 0.21 | −3.95 |
| | (Drosophila) | | | | | | |
| 218345_at | transmembrane protein 176A | TMEM176A | −0.27 | −1.05 | 0.30 | 0.63 | −5.90 |
| 218435_at | DnaJ (Hsp40) homolog, | DNAJC15 | −0.49 | −2.55 | 0.02 | 0.16 | −3.48 |
| | subfamily C, member 15 | | | | | | |
| 218545_at | coiled-coil domain containing | CCDC91 | −0.31 | −2.97 | 0.01 | 0.09 | −2.57 |
| | 91 | | | | | | |
| 218597_s_at | CDGSH iron sulfur domain 1 | CISD1 | −0.18 | −2.24 | 0.03 | 0.22 | −4.12 |
| 218648_at | CREB regulated transcription | CRTC3 | −0.33 | −3.39 | 0.00 | 0.05 | −1.58 |
| | coactivator 3 | | | | | | |
| 218651_s_at | La ribonucleoprotein domain | LARP6 | −0.34 | −4.00 | 0.00 | 0.02 | −0.03 |
| | family, member 6 | | | | | | |
| 218660_at | dysferlin, limb girdle muscular | DYSF | −0.55 | −3.49 | 0.00 | 0.04 | −1.33 |
| | dystrophy 2B (autosomal | | | | | | |
| | recessive) | | | | | | |
| 218668_s_at | RAP2C, member of RAS | RAP2C | −0.22 | −1.51 | 0.14 | 0.45 | −5.34 |
| | oncogene family | | | | | | |
| 218683_at | polypyrimidine tract binding | PTBP2 | −0.18 | −1.63 | 0.11 | 0.41 | −5.17 |
| | protein 2 | | | | | | |
| 218691_s_at | PDZ and LIM domain 4 | PDLIM4 | −0.42 | −2.50 | 0.02 | 0.16 | −3.58 |
| 218711_s_at | serum deprivation response | SDPR | 0.41 | 2.63 | 0.01 | 0.14 | −3.32 |
| | (phosphatidylserine binding | | | | | | |
| | protein) | | | | | | |
| 218818_at | four and a half LIM domains 3 | FHL3 | −0.36 | −2.29 | 0.03 | 0.21 | −4.02 |
| 218864_at | tensin 1 | TNS1 | −0.30 | −1.72 | 0.10 | 0.38 | −5.03 |
| 218877_s_at | tRNA methyltransferase 11 | TRMT11 | 0.44 | 2.93 | 0.01 | 0.10 | −2.66 |
| | homolog (S. cerevisiae) | | | | | | |
| 218975_at | collagen, type V, alpha 3 | COL5A3 | −0.32 | −1.79 | 0.08 | 0.35 | −4.91 |
| 219058_x_at | tubulointerstitial nephritis | TINAGL1 | −0.14 | −1.50 | 0.14 | 0.45 | −5.35 |
| | antigen-like 1 | | | | | | |
| 219073_s_at | oxysterol binding protein-like | OSBPL10 | −0.37 | −2.24 | 0.03 | 0.22 | −4.11 |
| | 10 | | | | | | |
| 219091_s_at | multimerin 2 | MMRN2 | −0.44 | −3.79 | 0.00 | 0.03 | −0.57 |
| 219102_at | reticulocalbin 3, EF-hand | RCN3 | −0.14 | −1.57 | 0.13 | 0.43 | −5.25 |
| | calcium binding domain | | | | | | |
| 219314_s_at | zinc finger protein 219 | ZNF219 | −0.51 | −4.66 | 0.00 | 0.01 | 1.70 |
| 219336_s_at | activating signal cointegrator 1 | ASCC1 | −0.16 | −1.59 | 0.12 | 0.42 | −5.23 |
| | complex subunit 1 | | | | | | |
| 219416_at | scavenger receptor class A, | SCARA3 | −0.57 | −2.45 | 0.02 | 0.18 | −3.71 |
| | member 3 | | | | | | |
| 219451_at | methionine sulfoxide reductase | MSRB2 | −0.42 | −2.07 | 0.05 | 0.27 | −4.43 |
| | B2 | | | | | | |
| 219488_at | alpha 1,4-galactosyltransferase | A4GALT | −0.14 | −1.56 | 0.13 | 0.43 | −5.26 |
| | (globotriaosylceramide | | | | | | |
| | synthase) | | | | | | |
| 219534_x_at | cyclin-dependent kinase | CDKN1C | −0.23 | −1.86 | 0.07 | 0.33 | −4.80 |
| | inhibitor 1C (p57, Kip2) | | | | | | |
| 219563_at | chromosome 14 open reading | C14orf139 | −0.38 | −2.33 | 0.03 | 0.20 | −3.95 |
| | frame 139 | | | | | | |
| 219656_at | protocadherin 12 | PCDH12 | −0.26 | −1.82 | 0.08 | 0.34 | −4.86 |
| 219689_at | sema domain, immunoglobulin | SEMA3G | −0.22 | −1.23 | 0.23 | 0.56 | −5.71 |
| | domain (Ig), short basic | | | | | | |
| | domain, secreted, | | | | | | |
| | (semaphorin) 3G | | | | | | |
| 219746_at | D4, zinc and double PHD | DPF3 | −0.18 | −1.66 | 0.11 | 0.40 | −5.12 |
| | fingers, family 3 | | | | | | |
| 219902_at | betaine-homocysteine | BHMT2 | −0.33 | −2.26 | 0.03 | 0.22 | −4.07 |
| | methyltransferase 2 | | | | | | |
| 219909_at | matrix metallopeptidase 28 | MMP28 | −0.54 | −3.44 | 0.00 | 0.05 | −1.45 |
| 220050_at | chromosome 9 open reading | C9orf9 | −0.32 | −2.10 | 0.04 | 0.26 | −4.37 |
| | frame 9 | | | | | | |
| 220091_at | solute carrier family 2 | SLC2A6 | −0.18 | −1.37 | 0.18 | 0.50 | −5.53 |
| | (facilitated glucose | | | | | | |
| | transporter), member 6 | | | | | | |
| 220103_s_at | mitochondrial ribosomal | MRPS18C | 0.21 | 1.82 | 0.08 | 0.34 | −4.87 |
| | protein S18C | | | | | | |
| 220148_at | aldehyde dehydrogenase 8 | ALDH8A1 | −0.45 | −1.58 | 0.12 | 0.43 | −5.23 |
| | family, member A1 | | | | | | |
| 220244_at | loss of heterozygosity, 3, | LOH3CR2A | 0.47 | 1.93 | 0.06 | 0.31 | −4.67 |
| | chromosomal region 2, gene A | | | | | | |
| 220276_at | RERG/RAS-like | RERGL | −0.54 | −1.75 | 0.09 | 0.37 | −4.98 |
| 220722_s_at | solute carrier family 5 (choline | SLC5A7 | −0.41 | −2.27 | 0.03 | 0.22 | −4.05 |
| | transporter), member 7 | | | | | | |
| 220765_s_at | LIM and senescent cell | LIMS2 | −0.41 | −2.81 | 0.01 | 0.11 | −2.93 |
| | antigen-like domains 2 | | | | | | |
| 220879_at | — | — | 0.20 | 2.17 | 0.04 | 0.24 | −4.25 |
| 220975_s_at | C1q and tumor necrosis factor | C1QTNF1 | −0.25 | −1.89 | 0.07 | 0.32 | −4.75 |
| | related protein 1 | | | | | | |
| 221014_s_at | RAB33B, member RAS | RAB33B | −0.38 | −2.47 | 0.02 | 0.17 | −3.66 |
| | oncogene family | | | | | | |
| 221030_s_at | Rho GTPase activating protein | ARHGAP24 | −0.27 | −1.66 | 0.11 | 0.40 | −5.11 |
| | 24 | | | | | | |
| 221127_s_at | regulated in glioma | RIG | −0.19 | −1.74 | 0.09 | 0.37 | −4.99 |
| 221193_s_at | zinc finger, CCHC domain | ZCCHC10 | −0.20 | −1.43 | 0.16 | 0.48 | −5.45 |
| | containing 10 | | | | | | |
| 221204_s_at | cartilage acidic protein 1 | CRTAC1 | −0.56 | −4.18 | 0.00 | 0.01 | 0.44 |
| 221246_x_at | tensin 1 | TNS1 | −0.27 | −3.41 | 0.00 | 0.05 | −1.53 |
| 221276_s_at | syncoilin, intermediate | SYNC1 | −0.29 | −1.63 | 0.11 | 0.41 | −5.17 |
| | filament 1 | | | | | | |
| 221447_s_at | glycosyltransferase 8 domain | GLT8D2 | 0.57 | 2.29 | 0.03 | 0.21 | −4.02 |
| | containing 2 | | | | | | |
| 221480_at | heterogeneous nuclear | HNRNPD | −0.36 | −2.27 | 0.03 | 0.22 | −4.06 |
| | ribonucleoprotein D (AU-rich | | | | | | |
| | element RNA binding protein | | | | | | |
| | 1, 37 kDa) | | | | | | |
| 221502_at | karyopherin alpha 3 (importin | KPNA3 | −0.20 | −2.16 | 0.04 | 0.24 | −4.26 |
| | alpha 4) | | | | | | |
| 221527_s_at | par-3 partitioning defective 3 | PARD3 | −0.16 | −1.59 | 0.12 | 0.42 | −5.23 |
| | homolog (C. elegans) | | | | | | |
| 221634_at | ribosomal protein L23a | RPL23AP7 | −0.21 | −2.04 | 0.05 | 0.28 | −4.48 |
| | pseudogene 7 | | | | | | |
| 221667_s_at | heat shock 22 kDa protein 8 | HSPB8 | −0.40 | −2.29 | 0.03 | 0.21 | −4.02 |
| 221748_s_at | tensin 1 | TNS1 | −0.14 | −1.62 | 0.12 | 0.41 | −5.18 |
| 221886_at | DENN/MADD domain | DENND2A | −0.33 | −1.83 | 0.08 | 0.34 | −4.84 |
| | containing 2A | | | | | | |
| 222066_at | Erythrocyte membrane protein | EPB41L1 | −0.20 | −1.76 | 0.09 | 0.36 | −4.97 |
| | band 4.1-like 1 | | | | | | |
| 222101_s_at | dachsous 1 (Drosophila) | DCHS1 | −0.26 | −1.56 | 0.13 | 0.43 | −5.27 |
| 222221_x_at | EH-domain containing 1 | EHD1 | −0.20 | −2.43 | 0.02 | 0.18 | −3.74 |
| 222257_s_at | angiotensin I converting | ACE2 | −0.38 | −1.96 | 0.06 | 0.30 | −4.62 |
| | enzyme (peptidyl-dipeptidase | | | | | | |
| | A) 2 | | | | | | |
| 32094_at | carbohydrate (chondroitin 6) | CHST3 | −0.19 | −1.09 | 0.29 | 0.62 | −5.86 |
| | sulfotransferase 3 | | | | | | |
| 32625_at | natriuretic peptide receptor | NPR1 | −0.22 | −2.46 | 0.02 | 0.17 | −3.68 |
| | A/guanylate cyclase A | | | | | | |
| | (atrionatriuretic peptide | | | | | | |
| | receptor A) | | | | | | |
| 336_at | thromboxane A2 receptor | TBXA2R | −0.65 | −3.37 | 0.00 | 0.05 | −1.62 |
| 33760_at | peroxisomal biogenesis factor | PEX14 | −0.24 | −1.74 | 0.09 | 0.37 | −5.00 |
| | 14 | | | | | | |
| 35776_at | intersectin 1 (SH3 domain | ITSN1 | −0.20 | −1.62 | 0.12 | 0.41 | −5.18 |
| | protein) | | | | | | |
| 35846_at | thyroid hormone receptor, | THRA | −0.46 | −3.87 | 0.00 | 0.02 | −0.38 |
| | alpha (erythroblastic leukemia | | | | | | |
| | viral (v-erb-a) oncogene | | | | | | |
| | homolog, avian) | | | | | | |
| 37996_s_at | dystrophia myotonica-protein | DMPK | −0.39 | −1.83 | 0.08 | 0.34 | −4.84 |
| | kinase | | | | | | |
| 38290_at | regulator of G-protein | RGS14 | −0.17 | −1.18 | 0.25 | 0.57 | −5.76 |
| | signaling 14 | | | | | | |
| 44702_at | synapse defective 1, Rho | SYDE1 | −0.38 | −2.45 | 0.02 | 0.18 | −3.69 |
| | GTPase, homolog 1 (C. elegans) | | | | | | |
| 45714_at | host cell factor C1 regulator 1 | HCFC1R1 | −0.24 | −1.29 | 0.21 | 0.53 | −5.63 |
| | (XPO1 dependent) | | | | | | |
| 52255_s_at | collagen, type V, alpha 3 | COL5A3 | −0.42 | −2.05 | 0.05 | 0.28 | −4.47 |

TABLE 14
146 diagnostic probe sets with incidence number greater than 50 for
105-fold gene selection procedure. The 15 shaded probe sets at the bottom
are deselected by PAM when the 146 probe sets
were used as input for training.

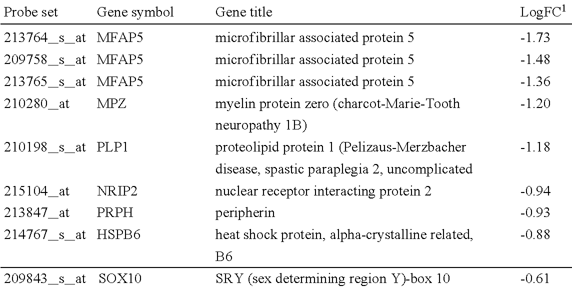

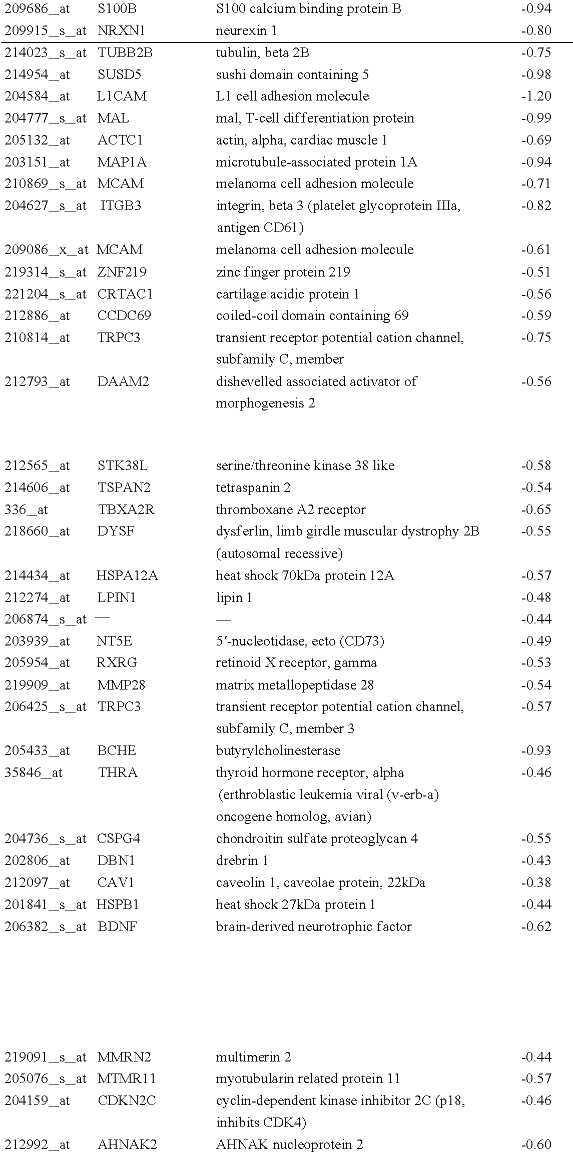

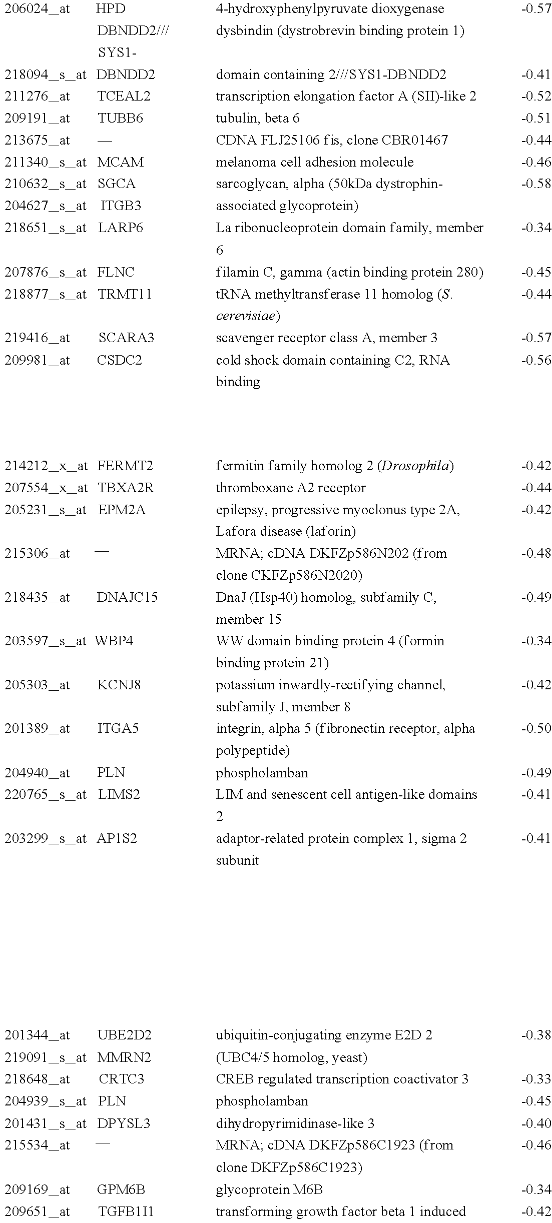

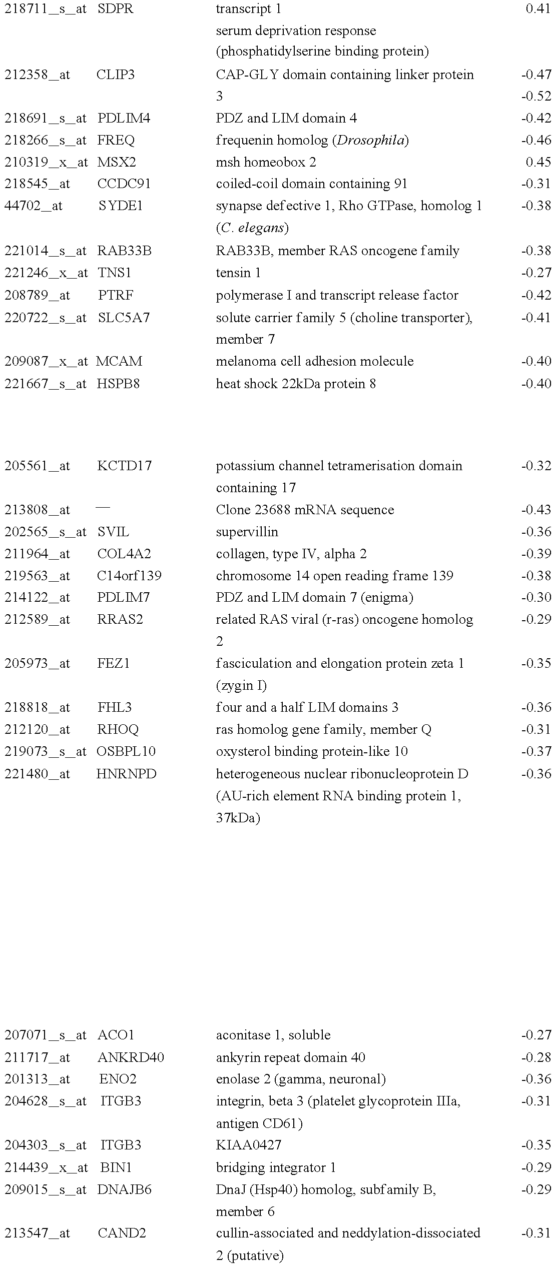

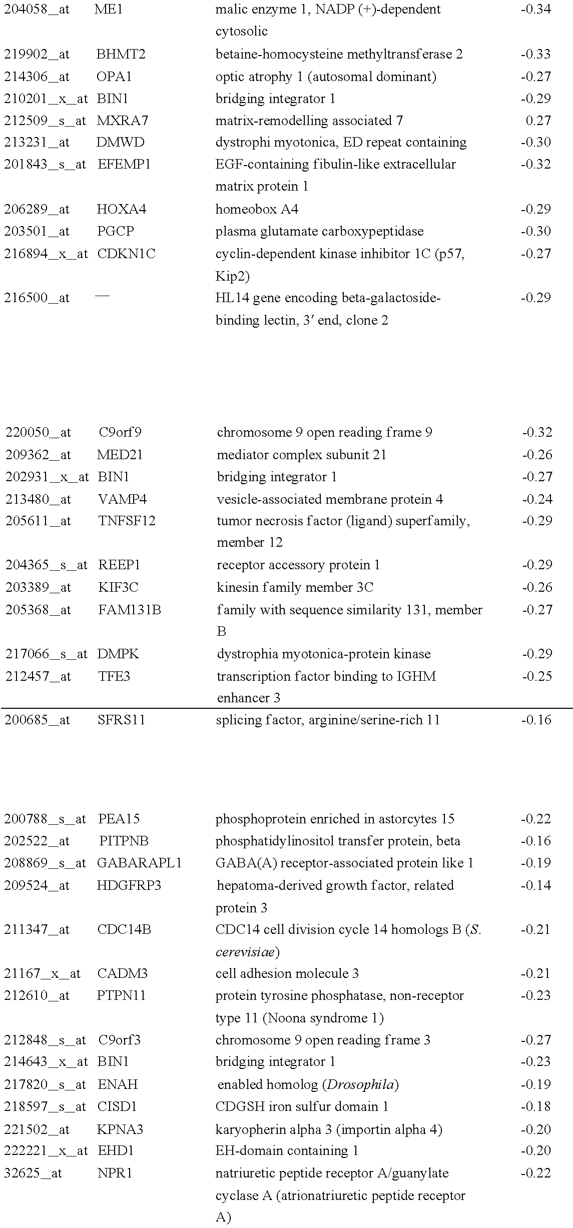

¹logFC is the logarithm Fold Change as tumorous stroma being compared to normal stroma. +/− represents up-/down- regulated expression level in tumorous stroma.

TABLE 5
Comparison of 131-element classifier to classifiers generated from
‘random’ genes. ‘i’ and ‘ii’ denote the 131-probeset
classifier and random-gene classifiers, respectively.

| | | | | Accuracy | | Sensitivity | | Specificity | |
|---|---|---|---|---|---|---|---|---|---|
| | | | | % | | % | | % | |
| | | Dataset | Case Num. | i | ii | i | ii | i | ii |
| 1 | Training set | 1 | 26 (13 + 13) | 96.4 | 67.1 | 92.3 | 32.5 | 100 | 97.1 |
| | Test set | | | | | | | | |
| | Tumor | | | | | | | | |
| 2 | Tumor-bearing | 1 | 55 (68 − 13) | 96.4 | 8.7 | 96.4 | 8.7 | NA | NA |
| 3 | Tumor-bearing | 2 | 65 | 100 | 12.9 | 100 | 12.9 | NA | NA |
| 4 | Tumor-bearing | 3 | 79 | 100 | 13.4 | 100 | 13.4 | NA | NA |
| 5 | Tumor-bearing | 4 | 44 | 100 | 15.9 | 100 | 15.9 | NA | NA |
| | Normal | | | | | | | | |
| 6 | Biopsies (1) | 1 | 7 | 100 | 98.8 | NA | NA | 100 | 98.8 |
| 7 | Biopsies (2) | 1 | 5 | 60.0 | 100 | NA | NA | 60.0 | 100 |
| 8 | Rapid autopsies | 1 | 13 | 92.3 | 67.5 | NA | NA | 92.3 | 67.5 |
| | Manuel | | | | | | | | |
| | Midrodissected/LCM | | | | | | | | |
| 9 | Tumor-adjacent | 2 | 71 | 97.1 | 13.6 | 97.1 | 13.6 | NA | NA |
| | Stroma | | | | | | | | |
| 10 | Tumor-adjacent | 4 | 13 | 100 | 15.9 | 100 | 15.9 | NA | NA |
| | Stroma | | | | | | | | |
| 11 | Tumor-adjacent | 1 | 12 | 75.0 | 5.8 | 75.0 | 5.8 | NA | NA |
| | Stroma | | | | | | | | |
| 12 | Tumor-bearing | 5 | 12 | 100 | 19.2 | 100 | 19.2 | NA | NA |
| 13 | Pooled normal | 5 | 4 | 100 | 79.4 | NA | NA | 100 | 79.4 |
| | stroma | | | | | | | | |

### Example 2

### Development of Predictive Biomarkers of Prostate Cancer

Three methods utilized in the development of predictive gene signature of prostate cancer are described in this example. First, an analytical method based on a linear combination model for the determination of the percent cell composition of the tumor epithelial cells and the stoma cells from array data of mixed cell type prostate tissue is described. The method utilizes fixed expression coefficients of a small (<100) genes that with expression characteristics that are distinct for tumor epithelial and stroma cells.

Second, a new method for the determination of tumor cell specific biomarkers for the prediction of relapse of prostate cancer using an extended linear combination model is described and validated. A gene profile based on the expression of RNA of prostate cancer epithelial cells that predicts the differential gene expression of relapse (aggressive) vs. non relapse (indolent) prostate cancer is derived. These genes are validated by their identification in independent sets of prostate cancer patients (technical retrospective validation) is described. This method may be used to identify aggressive prostate cancer from data obtained at the time of diagnosis. The method and profiles are novel.

Third, an analogous new method for the determination of stroma cell specific biomarkers for the prediction of relapse of prostate cancer is described. Thus the predictions are based on non tumor cell types. A gene profile based on the expression of RNA of stroma cells of tumor-bearing prostate tissue that predicts the differential gene expression of relapse (aggressive) vs. non relapse (indolent) prostate cancer that is validated by prediction of differences of an independent set of prostate cancer patients (technical retrospective validation) is described. These methods and profiles may be used to identify aggressive prostate cancer from data obtained at the time of diagnosis. The results further indicate that the microenvironment of tumor foci of prostate cancer exhibit altered gene expression at the time of diagnosis which is distinct in non relapse and relapsed prostate cancer.

Datasets: The goals of this study were to continue development of predicative biomarkers of prostate cancer. In particular the goal of this study is to use independent datasets to validate genes deduced as predictive based on studies of dataset 1 (infra vide). Here “dataset” refers to the array-based RNA expression data of all cases of a given set together with the clinical data defining whether a given case relapsed (recurred cancer) or remained disease free, a censored quantity. Only the categorical value, relapsed or non relapsed, is used in the analyses described here.

The three datasets used for this study included 1) 148 Affymetrix U133A array data acquired from 91 patients (publicly available in the GEO database as accession no. GSE8218) which is the principal dataset utilized in previous studies; 2) Illumina (of Illumina Inc., San Diego) beads arrays data from 103 patients as analyzed on 115 arrays, a published dataset (Bibilova et al. (2007) Genomics 89:666-672); and 3) Affymetrix U133A array data from 79 patients, also a published dataset (Stephenson et al., supra). These are referred to in this example as datasets 1, 2, and 3 respectively.

For the purposes herein, relapsed prostate cancer is taken as a surrogate of aggressive disease, while non-relapse is taken as indolent disease with a variable degree of indolence that is directly proportional to the disease-free survival time. Dataset 1 contains 40 non-relapse patients and 47 relapse patients; dataset 2 contains 75 non-relapse patients and 22 relapse patients, and dataset 3 contains 42 non-relapse patients and 37 relapse patients. The first two datasets samples have various amount of different tissue and cell types, including tumor cells, stroma cells (a collective term for fibroblasts, myofibroblasts, smooth muscle, and small amounts of nerve and vascular elements), BPH (epithelial cells of benign prostate hypertrophy) and dilated cystic glands (AKA “atrophic” cystic glands), as estimated by four pathologists (Stuart et al., supra) for dataset 1 and one pathologist for dataset 2. Dataset 3 samples were tumor-enriched samples. In this study, published datasets 2 and 3 were used for the purpose of validation only. A major goal of this study was to use “external” published datasets to validate the properties deduced for genes based on analysis of the dataset 1.

Determination of Cell Specific Gene Expression in Prostate Cancer: Using linear models applied to microarray data from prostate tissues with various amounts of different cell types as estimated by a team of four pathologists, identified genes were identified as being specifically expressed in different cell types (tumor, stroma, BPH and dilated cystic glands) of prostate tissue following published methods (Stuart et al., supra). Thus, the following linear models were applied for generating tissue specific genes.

Model 1—For any gene i, the hybridization intensity, Gᵢ, from an Affymetrix GeneChip is due to the sum of the cell contributions to the total mRNA:

Gᵢ=(βₜᵤₘₒᵣ·Pₜᵤₘₒᵣ+βₛₜᵣₒₘₐ·Pₛₜᵣₒₘₐβ_{BPH}·P_{BPH}β_{dilated cystic gland}·P_{dilated cystic gland})ᵢ

Where a “cell contribution” is the amount of the cellular component, P_{cell type}, multiplied times the characteristic expression level of gene i by that cell type, β. Only the β values are unknown and are determined by simple or multiple linear regressions. Note that in general a minimum of four estimates of Gᵢ (i.e. four cases) are required to estimate four unknown β whereas in practice many dozens of cases are available so that the unknown coefficients are “over determined”.

Model 2—Since the epithelia of dilated cystic glands were not a major component of prostate tissue, it may be removed from the linear model to simplify the model.

Gᵢ=(βₜᵤₘₒᵣ·Pₜᵤₘₒᵣ+βₛₜᵣₒₘₐ·Pₛₜᵣₒₘₐ+β_{BPH}·P_{BPH})ᵢ

Models 3˜6—To further simplify the model, cell composition also can be considered as two different cell types, usually one specific cell type and all the other cell types were grouped together.

Gᵢ=(βₜᵤₘₒᵣ·Pₜᵤₘₒᵣ+βₙₒₙ₋ₜᵤₘₒᵣ·Pₙₒₙ₋ₜᵤₘₒᵣ)ᵢ

Gᵢ=(βₛₜᵣₒₘₐ·Pₛₜᵣₒₘₐ+βₙₒₙ₋ₛₜᵣₒₘₐ·Pₙₒₙ₋ₛₜᵣₒₘₐ)ᵢ

Gᵢ=(β_{BPH}·P_{BPH}+β_{non-BPH}·P_{non-BPH})ᵢ

Gᵢ=(β_{dilated cystic gland}·P_{dilated cystic gland}+β_{non-dilated cystic gland}·P_{non-dilated cystic gland})ᵢ

The gene lists (with p<0.001) developed from models 3 and 4 using dataset 1 are listed in Table 6.

A New Method for Determination of Cell Type Composition Prediction Using Gene Expression Profiles Using linear models based on a small list of cell specific genes, i.e., genes from Table 6, the approximate percentage of cell types in samples hybridized to the array may be estimated using only the microarray data utilizing model 3. Potentially all of the genes in Table 6 can be used for cell percent composition prediction. For each individual gene, a new sample's gene expression value from microarray data can be fitted to models 3-6, for a prediction of corresponding cell type percentage. Each gene employed in model 3 provides an estimate of percent tumor cell composition. The median of the predictions based on multiple genes was used to generate a more reliable result estimate of tumor cell content. These prediction genes can be selected/ranked by either their correlation coefficient (for correlation between gene expression level and cell type percentage) or by combination of genes with the best prediction power. In the present case, only a very limited number of genes (8-52 genes) were used for such a prediction. Even fewer genes might be sufficient.

To validate the method of tumor or stroma percent composition determination, the known percent composition figures of dataset 1 were used to predict the tumor cell and stroma cell compositions for dataset 2 with known cell composition. For example, the number of genes used for cell type (tumor epithelial cells or stroma cells) prediction between dataset 1 and dataset 2 ranges from 8 to 52 genes, which are listed in Table 7A. The Pearson correlation coefficient between predicted cell type percentage (tumor epithelial cells or stroma cells) and pathologist estimated percentage ranged from 0.7 to 0.87. Tissue (tumor or stroma) specific genes identified from dataset 2 and used for prediction are listed in Table 7B.

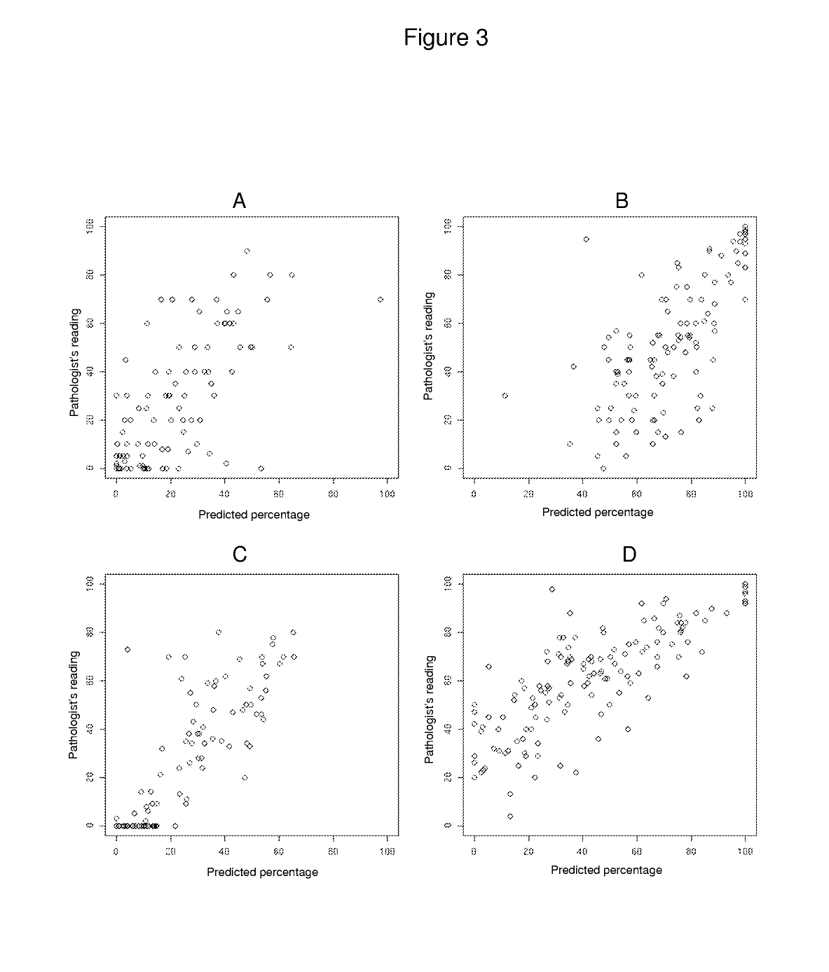

Since dataset 1 and dataset 2 data were based on different array platforms, the cross-platform normalization were applied using median rank scores (MRS) method (Warnat et al. (2005) BMC Bioinformatics 6:265). FIGS. 3A and 3B illustrate the use of the parameters of dataset 1 to predict the cell composition of dataset 2. The Pearson correlation coefficients for the correlation of the observed and calculated cell type compositions is 0.74 and 0.70 respectively. The converse calculations of utilizing the parameters of dataset 2 to calculate the tumor and stroma cell percent compositions of dataset 1 are shown in FIGS. 3C and 3D, respectively. The Pearson correlation coefficients were 0.87 and 0.78 respectively. The range of Pearson coefficients among four pathologists determined independently for composition estimates of the same samples in dataset 1 is 0.85-0.95 (Stuart et al., supra). Thus, the in silico estimates have a correlation that is almost completely subsumed in variation among pathologists, indicating that the in silico estimates are at least similar in performance to a pathologist and leaving open the possibility that the in silico estimates are more accurate than the pathologists.

A New Method for Determination of Cell Specific Relapse Related Genes of Prostate Cancer: Using dataset 1, the genes correlating with patient relapse status were estimated using the following linear models.

### Model 7

Gᵢ=β′_{tumor,i}Pₜᵤₘₒᵣ+β′_{stroma,i}Pₛₜᵣₒₘₐ+β′_{BPH,i}P_{BPH}+β′_{dilated cystic gland,i}P_{dilated cystic gland}+rs(γ_{tumor,i}Pₜᵤₘₒᵣ+γ_{stroma,i}Pₛₜᵣₒₘₐ+γ_{BPH,i}P_{BPH}+γ_{dilated cysstic gland,i}P_{dilated cystic gland})

For any gene i, Gᵢ (the array reported gene intensity)=the sum of 4 cell type contributions for non relapsed cases (β_{cell type,i}×Percent_{cell type})+Sum of 4 cell type contributions for relapsed cases (γ_{cell type,i}×Percent_{cell type})+error term. RS may be either 0 or 1 where 0 is utilized for all non relapse cases and RS=0 is utilized for relapse cases. Thus when RS=0 the expression coefficients β′ for non relapse cases are determined while when RS=1 the coefficients (β′+γ) are determined. Coefficients are numerically determined by multiple linear regression using least squares determination of best fit coefficients±error. The differences in expression between non relapse (β′) and relapse (β′+γ) is just γ and the significance y may be estimated by T-test and other standard statistical methods.

Model 8˜11—The following models also were implemented to simplify the models:

Gᵢ=β′_{tumor,i}Pₜᵤₘₒᵣ+β′_{relapse status,i}RS+β′_{int eraction,i}Pₜᵤₘₒᵣ:RS

Gᵢ=β′_{stroma,i}Pₛₜᵣₒₘₐ+β′_{relapse status,i}RS+β′_{int eraction,i}Pₛₜᵣₒₘₐ:RS

Gᵢ=β′_{Btumor,i}Pₜᵤₘₒᵣ+β′_{relapse status,i}RS+β′_{int eraction,i}Pₜᵤₘₒᵣ:RS

Gᵢ=β′_{dilated cystic gland,i}Pₜᵤₘₒᵣ+β′_{relapse status,i}RS+β′_{int eraction,i}P_{dilated cystic gland}:RS

Only the samples with >0% tumor epithelial cells were used for the above analysis to remove those far-stroma samples (i.e., non-tumor cell bearing samples). This exclusion of “far-stroma” accommodates the possibility that stroma may contain expression changes characteristic of prostates with cancer, but that these changes might be confined to stroma regions near tumor cells. Because multiple samples are used from some subjects, the estimating equations approach implemented in the “gee” library for R (i.e., the open source R bioinformatics analysis package) was used (Zeger and Liang (1986) Biometrics 42:121-130). Cell type (tumor epithelial cells or stroma cells) specific genes showed significant (p<0.005) expression level changes between relapse and non-relapse samples using model 8-9, are listed in Tables 8A and 8B.

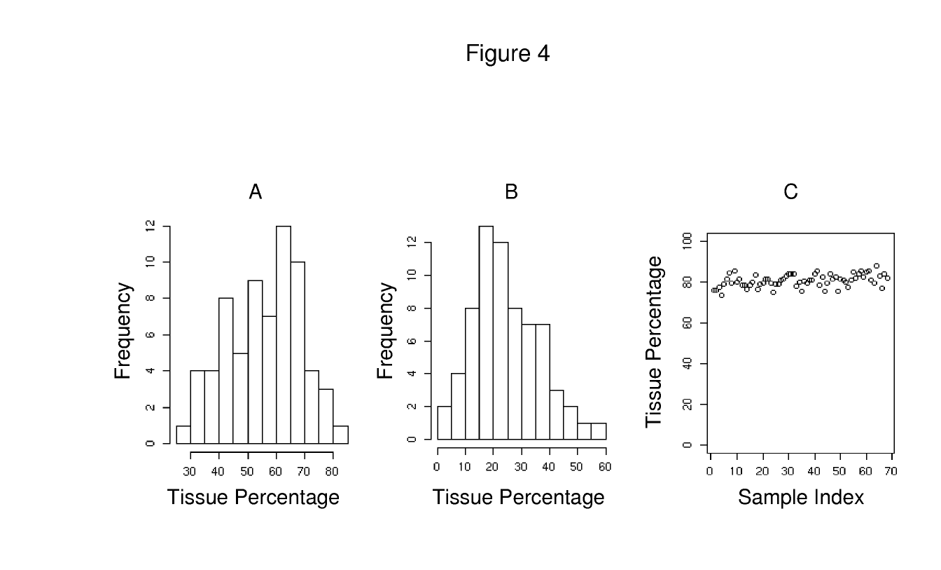

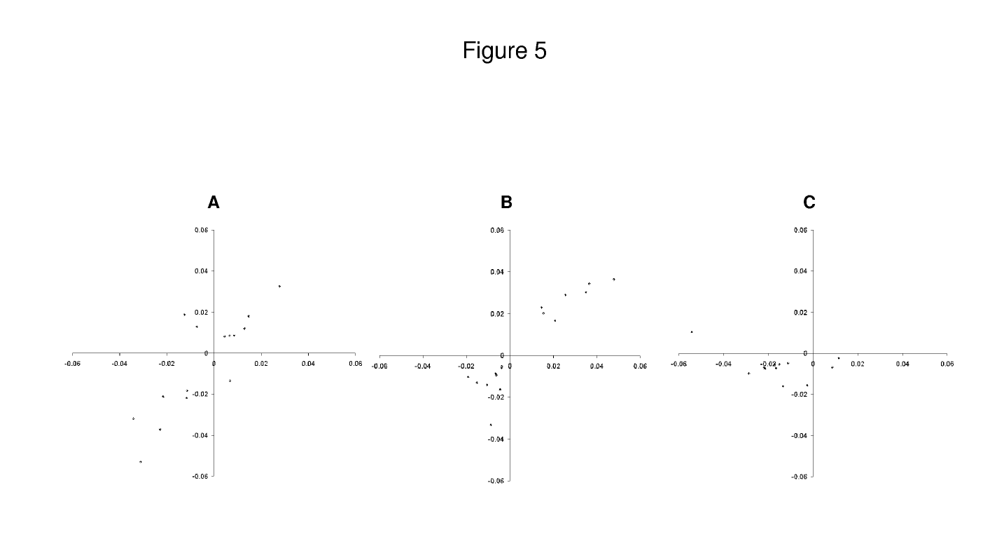

The gene list was then validated using independent dataset 3 to test whether any of the same genes were independently identified. Since dataset 3 has unknown tumor/stroma content, the method was first used for predicting tumor/stroma percentage (FIGS. 4A-4C) before testing the prediction potential of the genes of Tables 8A and 8B. Cell type (tumor epithelial cells or stroma cells) specific relapse related genes were generated using p<0.01 as a cut-off. There were 15 genes that were significantly associated with relapse in tumor cells in both datasets. Twelve genes agreed in identity and sign (direction in relapse). The null hypothesis that 12 genes agreeing and identity and sign was not different from random was tested, yielding a p<0.007. Thus these genes appear validated by the criterion of coincidence. The process is summarized in Table 9. These significant genes presented in both dataset 1 and 3 together with three additional genes that did not agree in sign between the two datasets are plotted in FIG. 5A which compares the expression coefficients for these genes in both datasets. Almost all of these genes showed consistency between two datasets, with a Pearson Correlation Coefficient of 0.83. Thus the coincident genes also agree in amplitude. These genes are listed in Table 10.

An analogous analysis was carried for the determination of stroma cell specific genes (FIG. 5B, Table 9). Sixteen genes exhibited correlation with relapse in both datasets, and all of these genes had the same direction in both datasets (p<0.001). The 16 genes exhibit a Pearson Correlation Coefficient of 0.93. This result indicates that a stroma cell based classifier may have predictive information about relapse. These genes determined from the analysis of datasets 1 and 3 are listed in Table 11.

An analogous analysis was carried out using datasets 1 and 2 with a significance cut off of 0.2 for dataset 2 (Table 9). Thirteen coincident genes were identified at this threshold even though the array of dataset three is relatively small (˜500 genes). Ten of these 13 genes had the same direction in relapse in both datasets (p<0.011), as shown in FIG. 5C. Thus, these 10 genes are validated in an independent dataset by the criterion of coincidence in independent datasets. The common 10 genes which had the same direction are listed in Table 12. One gene, PPAP2B (Affymetrix ID: 212230_at) is down-regulated in relapse cases and is in common with those of datasets 1 and 2.

A similar analysis for stroma-specifically expressed genes revealed BTG2 as a stroma specific relapse gene (Affymetrix ID: 201235_s_at) as a common gene in dataset 1 and 2 that exhibited up-regulation in both datasets.

These results indicate that three sets of validated genes with significant differential expression may be extracted once tumor percentage is taken into account, which may be useful in the prediction of relapse by analysis of expression data obtained at the time of diagnosis.

TABLE 6
Tissue Specific Genes detected using dataset 1 (p < 0.005).
Regular font: up-regulated genes; Italics: down-regulated genes.

| Tumor Specific Genes | Stroma Specific Genes | | |
|---|---|---|---|
| | | 36830_at | 202555_s_at |
| 209424_s_at | 201496_x_at | 203954_x_at | 212730_at |
| 209426_s_at | 208792_s_at | 212449_s_at | 203903_s_at |
| 209425_at | 213068_at | 212445_s_at | 214505_s_at |
| 219360_s_at | 205242_at | 209398_at | 205935_at |
| 203242_s_at | 208791_at | 204875_s_at | 211276_at |
| 221577_x_at | 201058_s_at | 205542_at | 219167_at |
| 216804_s_at | 202222_s_at | 209114_at | 205564_at |
| 204934_s_at | 213746_s_at | 218638_s_at | 204135_at |
| 209813_x_at | 205382_s_at | 209340_at | 209283_at |
| 211144_x_at | 204083_s_at | 217979_at | 207876_s_at |
| 204623_at | 222043_at | 219736_at | 202409_at |
| 215806_x_at | 203413_at | 214774_x_at | 219478_at |
| 203953_s_at | 203186_s_at | 218835_at | 209291_at |
| 221424_s_at | 212865_s_at | 219312_s_at | 208131_s_at |
| 216920_s_at | 218087_s_at | 204973_at | 212843_at |
| 205860_x_at | 213071_at | 221582_at | 209210_s_at |
| 203196_at | 214027_x_at | 206302_s_at | 209292_at |
| 205347_s_at | 210299_s_at | 203397_s_at | 203851_at |
| 217771_at | 202992_at | 203007_x_at | 200953_s_at |
| 215363_x_at | 212233_at | 214469_at | 201431_s_at |
| 211303_x_at | 201539_s_at | 220192_x_at | 202565_s_at |
| 202345_s_at | 212992_at | 205780_at | 203065_s_at |
| 217487_x_at | 203296_s_at | 204305_at | 210002_at |
| 203243_s_at | 210298_x_at | 209623_at | 203324_s_at |
| 206858_s_at | 201495_x_at | 201690_s_at | 215813_s_at |
| 214598_at | 207977_s_at | 214455_at | 209616_s_at |
| 203908_at | 203766_s_at | 204141_at | 210139_s_at |
| 209624_s_at | 214752_x_at | 221669_s_at | 202269_x_at |
| 212412_at | 209763_at | 209696_at | 209156_s_at |
| 213506_at | 217897_at | 216623_x_at | 200906_s_at |
| 218313_s_at | 207390_s_at | 203304_at | 205549_at |
| 201689_s_at | 221667_s_at | 214087_s_at | 208937_s_at |
| 203216_s_at | 204273_at | 205645_at | 202270_at |
| 201839_s_at | 221747_at | 202454_s_at | 212724_at |
| 212218_s_at | 200859_x_at | 213622_at | 200762_at |
| 206558_at | 209170_s_at | 202427_s_at | 201667_at |
| 201688_s_at | 212097_at | 214463_x_at | 217728_at |
| 205776_at | 203951_at | 219856_at | 203323_at |
| 220014_at | 213371_at | 200790_at | 213428_s_at |
| 208579_x_at | 208790_s_at | 205597_at | 212067_s_at |
| 201923_at | 222162_s_at | 210339_s_at | 209351_at |
| 206214_at | 217757_at | 210377_at | 209687_at |
| 203644_s_at | 209651_at | 217850_at | 201842_s_at |
| 204776_at | 210869_s_at | 200862_at | 218730_s_at |
| 46323_at | 200621_at | 203857_s_at | 212977_at |
| 219667_s_at | 204939_s_at | 204170_s_at | 203706_s_at |
| 212686_at | 202202_s_at | 201596_x_at | 209496_at |
| 200644_at | 200907_s_at | 219127_at | 209948_at |
| 216905_s_at | 209209_s_at | 201079_at | 201147_s_at |
| 202890_at | 201615_x_at | 212789_at | 201540_at |
| 204714_s_at | 201105_at | 222121_at | 213994_s_at |
| 200935_at | 202274_at | 209844_at | 204931_at |
| 205830_at | 205128_x_at | 203917_at | 219685_at |
| 218280_x_at | 209355_s_at | 204667_at | 209487_at |
| 217111_at | 205547_s_at | 218922_s_at | 211966_at |
| 201952_at | 209427_at | 211596_s_at | 202748_at |
| 222277_at | 203423_at | 220933_s_at | 218418_s_at |
| 212640_at | 221748_s_at | 208580_x_at | 214247_s_at |
| 203911_at | 203729_at | 218186_at | 206332_s_at |
| 210738_s_at | 214091_s_at | 217912_at | 201641_at |
| 206239_s_at | 204894_s_at | 214290_s_at | 209488_s_at |
| 208837_at | 200931_s_at | 212812_at | 202283_at |
| 202043_s_at | 206116_s_at | 211137_s_at | 204345_at |
| 221732_at | 207957_s_at | 202148_s_at | 209167_at |
| 201014_s_at | 201957_at | 204942_s_at | 209540_at |
| 219584_at | 213139_at | 209369_at | 218718_at |
| 215017_s_at | 202007_at | 215726_s_at | 213093_at |
| 210317_s_at | 201150_s_at | 214651_s_at | 211964_at |
| 203474_at | 218980_at | 204389_at | 212226_s_at |
| 213492_at | 205132_at | 219017_at | 211896_s_at |
| 203739_at | 215016_x_at | 213148_at | 209074_s_at |
| 210787_s_at | 204069_at | 219118_at | 218611_at |
| 210337_s_at | 202920_at | 215779_s_at | 203881_s_at |
| 211689_s_at | 200986_at | 87100_at | 201616_s_at |
| 212252_at | 205475_at | 213943_at | 202995_s_at |
| 201413_at | 208966_x_at | 220926_s_at | 200897_s_at |
| 202457_s_at | 221935_s_at | 212680_x_at | 207480_s_at |
| 220161_s_at | 202566_s_at | 214404_x_at | 202196_s_at |
| 215432_at | 201348_at | 209935_at | 209288_s_at |
| 217973_at | 219295_s_at | 201761_at | 217767_at |
| 202429_s_at | 204288_s_at | 205309_at | 221505_at |
| 208180_s_at | 200930_s_at | 209031_at | 201497_x_at |
| 204394_at | 212254_s_at | 209806_at | 209541_at |
| 215108_x_at | 204570_at | 220116_at | 204041_at |
| 210108_at | 203498_at | 200969_at | 218380_at |
| 210480_s_at | 209286_at | 208490_x_at | 200600_at |
| 218254_s_at | 212136_at | 202740_at | 209621_s_at |
| 219405_at | 201787_at | 209825_s_at | 209087_x_at |
| 201662_s_at | 212813_at | 203485_at | 205384_at |
| 204388_s_at | 203562_at | 207980_s_at | 201313_at |
| 206110_at | 208789_at | 210788_s_at | 212887_at |
| 201951_at | 204731_at | 208527_x_at | 212187_x_at |
| 220380_at | 209191_at | 213246_at | 208637_x_at |
| 205505_at | 209335_at | 218189_s_at | 202073_at |
| 200700_s_at | 209118_s_at | 221019_s_at | 204364_s_at |
| 204485_s_at | 206434_at | 209030_s_at | 212361_s_at |
| 202790_at | 204463_s_at | 219152_at | 201645_at |
| 202668_at | 214265_at | 214106_s_at | 212230_at |
| 212281_s_at | 201430_s_at | 213285_at | 213524_s_at |
| 204319_s_at | 207030_s_at | 207843_x_at | 212091_s_at |
| 201417_at | 200982_s_at | 217736_s_at | 203705_s_at |
| 204751_x_at | 208747_s_at | 202503_s_at | 202760_s_at |
| 206303_s_at | 202994_s_at | 210222_s_at | 205433_at |
| 215071_s_at | 204734_at | 202770_s_at | 207826_s_at |
| 202786_at | 213992_at | 203219_s_at | 209356_x_at |
| 221802_s_at | 220595_at | 202525_at | 218974_at |
| 209459_s_at | 209469_at | 213143_at | 209129_at |
| 217080_s_at | 211340_s_at | 222067_x_at | 219935_at |
| 202241_at | 202440_s_at | 201848_s_at | 213400_s_at |
| 213325_at | 204457_s_at | 218025_s_at | 207836_s_at |
| 213587_s_at | 207961_x_at | 213812_s_at | 204753_s_at |
| 201128_s_at | 204284_at | 222075_s_at | 216598_s_at |
| 214446_at | 201843_s_at | 210719_s_at | 203370_s_at |
| 212295_s_at | 204955_at | 210328_at | 201617_x_at |
| 201577_at | 214212_x_at | 202061_s_at | 220765_s_at |
| 210130_s_at | 203710_at | 218188_s_at | 211813_x_at |
| 219117_s_at | 201061_s_at | 200656_s_at | 202729_s_at |
| 209094_at | 204472_at | 202769_at | 201242_s_at |
| 211559_s_at | 201438_at | 221589_s_at | 204396_s_at |
| 209504_s_at | 204464_s_at | 202605_at | 203131_at |
| 208546_x_at | 204938_s_at | 204231_s_at | 212886_at |
| 201849_at | 218224_at | 201013_s_at | 212288_at |
| 202722_s_at | 211562_s_at | 221782_at | 206938_at |
| 74694_s_at | 220532_s_at | 207824_s_at | 204424_s_at |
| 212745_s_at | 212993_at | 217875_s_at | 214266_s_at |
| 214765_s_at | 204940_at | 218931_at | 204036_at |
| 222209_s_at | 205934_at | 209836_x_at | 211980_at |
| 205924_at | 201631_s_at | 218979_at | 209047_at |
| 220187_at | 202177_at | 213085_s_at | 202719_s_at |
| 219806_s_at | 210078_s_at | 211576_s_at | 206070_s_at |
| 213892_s_at | 206433_s_at | 205248_at | 213338_at |
| 202005_at | 201792_at | 215380_s_at | 217764_s_at |
| 202687_s_at | 204030_s_at | 201582_at | 200696_s_at |
| 203716_s_at | 213258_at | 201724_s_at | 219090_at |
| 203138_at | 209685_s_at | 202826_at | 204359_at |
| 212744_at | 202133_at | 209113_s_at | 203680_at |
| 202089_s_at | 200974_at | 203430_at | 218094_s_at |
| 221781_s_at | 212713_at | 212694_s_at | 209470_s_at |
| 209366_x_at | 202350_s_at | 219555_s_at | 211748_x_at |
| 213712_at | 213293_s_at | 219518_s_at | 212736_at |
| 211724_x_at | 213800_at | 202088_at | 221760_at |
| 219395_at | 203603_s_at | 201543_s_at | 212509_s_at |
| 203180_at | 209583_s_at | 206352_s_at | 206701_x_at |
| 218909_at | 212764_at | 221561_at | 205407_at |
| 205133_s_at | 204964_s_at | 219476_at | 218162_at |
| 205769_at | 204602_at | 203029_s_at | 211343_s_at |
| 212115_at | 213572_s_at | 200806_s_at | 209663_s_at |
| 218258_at | 205157_s_at | 218027_at | 200911_s_at |
| 200078_s_at | 212423_at | 209460_at | 212236_x_at |
| 221865_at | 217763_s_at | 217901_at | 203748_x_at |
| 205003_at | 204963_at | 201890_at | 212848_s_at |
| 205566_at | 221584_s_at | 219649_at | 200795_at |
| 207098_s_at | 213568_at | 219388_at | 206580_s_at |
| 201760_s_at | 209868_s_at | 212183_at | 200824_at |
| 221923_s_at | 213924_at | 213106_at | 218934_s_at |
| 213288_at | 211981_at | 216483_s_at | 214761_at |
| 218248_at | 209655_s_at | 210541_s_at | 222108_at |
| 201912_s_at | 204163_at | 210652_s_at | 200808_s_at |
| 212310_at | 201893_x_at | 219015_s_at | 202393_s_at |
| 200903_s_at | 214039_s_at | 210293_s_at | 211864_s_at |
| 212255_s_at | 213010_at | 219266_at | 200878_at |
| 222258_s_at | 201560_at | 202688_at | 206377_at |
| 206860_s_at | 209101_at | 214243_s_at | 202664_at |
| 201583_s_at | 217437_s_at | 204957_at | 37996_s_at |
| 203386_at | 217762_s_at | 218140_x_at | 212624_s_at |
| 201127_s_at | 208029_s_at | 207260_at | 211663_x_at |
| 204567_s_at | 202403_s_at | 212543_at | 212354_at |
| 202893_at | 212135_s_at | 205757_at | 209612_s_at |
| 218035_s_at | 205725_at | 201735_s_at | 218518_at |
| 203642_s_at | 206631_at | 212448_at | 204777_s_at |
| 217752_s_at | 212551_at | 208658_at | 202732_at |
| 209585_s_at | 201798_s_at | 200970_s_at | 204072_s_at |
| 202929_s_at | 201820_at | 212978_at | 209200_at |
| 208190_s_at | 209613_s_at | 209854_s_at | 210986_s_at |
| 221754_s_at | 202075_s_at | 213555_at | 212419_at |
| 203030_s_at | 202822_at | 209693_at | 212914_at |
| 205942_s_at | 207266_x_at | 221927_s_at | 221127_s_at |
| 203931_s_at | 221276_s_at | 202489_s_at | 212358_at |
| 209934_s_at | 200923_at | 204121_at | 208430_s_at |
| 209302_at | 212667_at | 201563_at | 213564_x_at |
| 204026_s_at | 204223_at | 202363_at | 209337_at |
| 40093_at | 205200_at | 220432_s_at | 202728_s_at |
| 210041_s_at | 201462_at | 204238_s_at | 211985_s_at |
| 218696_at | 210987_x_at | 212816_s_at | 213001_at |
| 209367_at | 208370_s_at | 205937_at | 219064_at |
| 202871_at | 201109_s_at | 215794_x_at | 212647_at |
| 209478_at | 204442_x_at | 208523_x_at | 209550_at |
| 205052_at | 204400_at | 207431_s_at | 219747_at |
| 205155_s_at | 213675_at | 205833_s_at | 212344_at |
| 206385_s_at | 210764_s_at | 214097_at | 221872_at |
| 222216_s_at | 205803_s_at | 212181_s_at | 209883_at |
| 200971_s_at | 211160_x_at | 212563_at | 218901_at |
| 200832_s_at | 208944_at | 222125_s_at | 201603_at |
| 221027_s_at | 211538_s_at | 202599_s_at | 214696_at |
| 218388_at | 216474_x_at | 200698_at | 214104_at |
| 203663_s_at | 206211_at | 204416_x_at | 201300_s_at |
| 201704_at | 204754_at | 221024_s_at | 205083_at |
| 217919_s_at | 204793_at | 218605_at | 213262_at |
| 202941_at | 204037_at | 216251_s_at | 205404_at |
| 218194_at | 209821_at | 211494_s_at | 203921_at |
| 203011_at | 201215_at | 212474_at | 201030_x_at |
| 222140_s_at | 205792_at | 201892_s_at | 202949_s_at |
| 218039_at | 201841_s_at | 217851_s_at | 58780_s_at |
| 212916_at | 204352_at | 210720_s_at | 210072_at |
| 213900_at | 201389_at | 211715_s_at | 213438_at |
| 202721_s_at | 211323_s_at | 213280_at | 214071_at |
| 219121_s_at | 209656_s_at | 203557_s_at | 203638_s_at |
| 221880_s_at | 213993_at | 214437_s_at | 212646_at |
| 209357_at | 202686_s_at | 218789_s_at | 204748_at |
| 222315_at | 219179_at | 202889_x_at | 211564_s_at |
| 202286_s_at | 219440_at | 217986_s_at | 209264_s_at |
| 214733_s_at | 205573_s_at | 201219_at | 214077_x_at |
| 209163_at | 203570_at | 200852_x_at | 221900_at |
| 200052_s_at | 221541_at | 50400_at | 209154_at |
| 202546_at | 203088_at | 220606_s_at | 212104_s_at |
| 200894_s_at | 202759_s_at | 203228_at | 207016_s_at |
| 203966_s_at | 211535_s_at | 218961_s_at | 221814_at |
| 211935_at | 212190_at | 201943_s_at | 203640_at |
| 212282_at | 218223_s_at | 212116_at | 201601_x_at |
| 206351_s_at | 212845_at | 203164_at | 213004_at |
| 213410_at | 203810_at | 203641_s_at | 206391_at |
| 200946_x_at | 201426_s_at | 212692_s_at | 203254_s_at |
| 209917_s_at | 211126_s_at | 209694_at | 205683_x_at |
| 218556_at | 213974_at | 209911_x_at | 201170_s_at |
| 218654_s_at | 202551_s_at | 218211_s_at | 212501_at |
| 200807_s_at | 205856_at | 218218_at | 201151_s_at |
| 206770_s_at | 217890_s_at | 203616_at | 209436_at |
| 212347_x_at | 204802_at | 206502_s_at | 218499_at |
| 202718_at | 212675_s_at | 206170_at | 218204_s_at |
| 219411_at | 823_at | 201416_at | 209285_s_at |
| 201647_s_at | 206392_s_at | 218888_s_at | 207134_x_at |
| 217942_at | 218711_s_at | 51158_at | 219654_at |
| 200681_at | 213503_x_at | 200670_at | 203295_s_at |
| 209531_at | 201329_s_at | 203215_s_at | 216733_s_at |
| 207414_s_at | 203620_s_at | 211297_s_at | 212274_at |
| 210547_x_at | 214724_at | 219065_s_at | 204497_at |
| 204331_s_at | 221755_at | 209389_x_at | 210427_x_at |
| 208788_at | 208636_at | 204175_at | 209169_at |
| 208737_at | 201590_x_at | 206429_at | 218330_s_at |
| 203041_s_at | 205127_at | 217749_at | 202766_s_at |
| 208398_s_at | 203571_s_at | 218592_s_at | 204749_at |
| 221345_at | 203688_at | 217809_at | 209473_at |
| 203387_s_at | 210517_s_at | 221590_s_at | 219647_at |
| 207949_s_at | 209897_s_at | 218261_at | 201387_s_at |
| 205925_s_at | 209406_at | 209916_at | 218824_at |
| 203224_at | 201559_s_at | 205698_s_at | 215382_x_at |
| 208802_at | 211737_x_at | 218387_s_at | 201060_x_at |
| 218883_s_at | 57588_at | 210715_s_at | 212805_at |
| 210024_s_at | 212535_at | 218465_at | 217996_at |
| 202836_s_at | 201536_at | 207606_s_at | 209466_x_at |
| 214875_x_at | 209465_x_at | 209605_at | 212677_s_at |
| 215696_s_at | 221676_s_at | 222262_s_at | 213982_s_at |
| 203593_at | 204621_s_at | 220625_s_at | 210145_at |
| 212186_at | 212566_at | 222155_s_at | 211984_at |
| 202109_at | 202086_at | | AFFX- |
| 218865_at | 204422_s_at | 202064_s_at | HSAC07/X00351_5_at |
| 201401_s_at | 206932_at | 204127_at | 201289_at |
| 205042_at | 207547_s_at | 201825_s_at | 207574_s_at |
| 201579_at | 204058_at | 218582_at | 213290_at |
| 219276_x_at | 203637_s_at | 215471_s_at | 1598_g_at |
| 211498_s_at | 204688_at | 202939_at | 202794_at |
| 201268_at | 213005_s_at | 218557_at | 219410_at |
| 201900_s_at | 219922_s_at | 219166_at | 202762_at |
| 211404_s_at | 212554_at | 205768_s_at | 213156_at |
| 209149_s_at | 204114_at | 209759_s_at | 204099_at |
| 217803_at | 212203_x_at | 209502_s_at | 214022_s_at |
| 212160_at | 205802_at | 220547_s_at | 202898_at |
| 212741_at | 209959_at | 204608_at | 208962_s_at |
| 203115_at | 209287_s_at | 205078_at | 221583_s_at |
| 218608_at | 213194_at | 218531_at | 202796_at |
| 211048_s_at | 210095_s_at | 217043_s_at | 201148_s_at |
| 218275_at | 218285_s_at | 202279_at | 202157_s_at |
| 203009_at | 201867_s_at | 211070_x_at | 208228_s_at |
| 218086_at | 208690_s_at | 217894_at | 201069_at |
| 218434_s_at | 202554_s_at | 201660_at | 215388_s_at |
| 204052_s_at | 201602_s_at | 203594_at | 202720_at |
| 201940_at | 212489_at | 219115_s_at | 205381_at |
| 203765_at | 209305_s_at | 200652_at | 65718_at |
| 204905_s_at | 211965_at | 217823_s_at | 212526_at |
| 204233_s_at | 203892_at | 212989_at | 203002_at |
| 215438_x_at | 209135_at | 201963_at | 210084_x_at |
| 37117_at | 204271_s_at | 200825_s_at | 203636_at |
| 219038_at | 205304_s_at | 221941_at | 218678_at |
| 202183_s_at | 209542_x_at | 91816_f_at | 218963_s_at |
| 219133_at | 201315_x_at | 218049_s_at | 218694_at |
| 221823_at | 209645_s_at | 209665_at | 202388_at |
| 207981_s_at | 201037_at | 220638_s_at | 204149_s_at |
| 203545_at | 205608_s_at | 203630_s_at | 218864_at |
| 212064_x_at | 201328_at | 205102_at | 209199_s_at |
| 218145_at | 205743_at | 209706_at | 201655_s_at |
| 218676_s_at | 216331_at | 201486_at | 217023_x_at |
| 220226_at | 206117_at | 208583_x_at | 219829_at |
| 201115_at | 203411_s_at | 208910_s_at | 206874_s_at |
| 221586_s_at | 205265_s_at | 210241_s_at | 211577_s_at |
| 220642_x_at | 206359_at | 213996_at | 201042_at |
| 203775_at | 212817_at | 204143_s_at | 204418_x_at |
| 201734_at | 201136_at | 202655_at | 208965_s_at |
| 221648_s_at | 202499_s_at | 214109_at | 216264_s_at |
| 212307_s_at | 204803_s_at | 215125_s_at | 209242_at |
| 212204_at | 202609_at | 208796_s_at | 218051_s_at |
| 209625_at | 202404_s_at | 213600_at | 215464_s_at |
| 209600_s_at | 202587_s_at | 214240_at | 203884_s_at |
| 203225_s_at | 216887_s_at | 211971_s_at | 213016_at |
| 200654_at | 216321_s_at | 217483_at | 218368_s_at |
| 206656_s_at | 221729_at | 221882_s_at | 219506_at |
| 207549_x_at | 207191_s_at | 218996_at | 213656_s_at |
| 208787_at | 201482_at | 200895_s_at | 212151_at |
| 213441_x_at | 200904_at | 205420_at | 201719_s_at |
| 203524_s_at | 202465_at | 219819_s_at | 205168_at |
| 202778_s_at | 204059_s_at | 207275_s_at | 209304_x_at |
| 212652_s_at | 201243_s_at | 221931_s_at | 214121_x_at |
| 222118_at | 204268_at | 204066_s_at | 219427_at |
| 200863_s_at | 209447_at | 201516_at | 204929_s_at |
| 204404_at | 221773_at | 210243_s_at | 221718_s_at |
| 209265_s_at | 218421_at | 217826_s_at | 212669_at |
| 201520_s_at | 202074_s_at | 208702_x_at | 212353_at |
| 211899_s_at | 207542_s_at | 201976_s_at | 218502_s_at |
| 210996_s_at | 210105_s_at | 214710_s_at | 201868_s_at |
| 209036_s_at | 202401_s_at | 212573_at | 212793_at |
| 201091_s_at | 202917_s_at | 218458_at | 204304_s_at |
| 208840_s_at | 201149_s_at | 217871_s_at | 201272_at |
| 214919_s_at | 212077_at | 212749_s_at | 215127_s_at |
| 212774_at | 204865_at | 203207_s_at | 208949_s_at |
| 203431_s_at | 209318_x_at | 219217_at | 213274_s_at |
| 202395_at | 204755_x_at | 217908_s_at | 202504_at |
| 218423_x_at | 201153_s_at | 200093_s_at | 201869_s_at |
| 218792_s_at | 218298_s_at | 201264_at | 201508_at |
| 215227_x_at | 210471_s_at | 216074_x_at | 209205_s_at |
| 218073_s_at | 212488_at | 211747_s_at | 213411_at |
| 218969_at | 215707_s_at | 209593_s_at | 203973_s_at |
| 201947_s_at | 202071_at | 213059_at | 203607_at |
| 209905_at | 221766_s_at | 219787_s_at | 211719_x_at |
| 212279_at | 208816_x_at | 201691_s_at | 203725_at |
| 203284_s_at | 203140_at | 200968_s_at | 213275_x_at |
| 203517_at | 204115_at | 204168_at | 213714_at |
| 201066_at | 219505_at | 201075_s_at | 212240_s_at |
| 209224_s_at | 201369_s_at | 208612_at | 202132_at |
| 213244_at | 222101_s_at | 208918_s_at | 201008_s_at |
| 220030_at | 209293_x_at | 218439_s_at | 91703_at |
| 203139_at | 212587_s_at | 212922_s_at | 205051_s_at |
| 218984_at | 211962_s_at | 205293_x_at | 221796_at |
| 211549_s_at | 210896_s_at | 218291_at | 212253_x_at |
| 202918_s_at | 212757_s_at | 216305_s_at | 205303_at |
| 201088_at | 45297_at | 221739_at | 209086_x_at |
| 202961_s_at | 206458_s_at | 202418_at | 205620_at |
| 218001_at | 204990_s_at | 206299_at | 209298_s_at |
| 218500_at | 201152_s_at | 218206_x_at | 207741_x_at |
| 202428_x_at | 221246_x_at | 64486_at | 212195_at |
| 220753_s_at | 214464_at | 209776_s_at | 202411_at |
| 220892_s_at | 221045_s_at | 212165_at | 214660_at |
| 201736_s_at | 212464_s_at | 218704_at | 218486_at |
| 208309_s_at | 222288_at | 218944_at | 203939_at |
| 218966_at | 201235_s_at | 214214_s_at | 212276_at |
| 213308_at | 210036_s_at | 203102_s_at | 209307_at |
| 201722_s_at | 203325_s_at | 211733_x_at | 201958_s_at |
| 205807_s_at | 212430_at | 214096_s_at | 213364_s_at |
| 202660_at | 212086_x_at | 219215_s_at | 220751_s_at |
| 202606_s_at | 218435_at | 210396_s_at | 213381_at |
| 39817_s_at | 202724_s_at | 202138_x_at | 222303_at |
| 214157_at | 207002_s_at | 212570_at | 203753_at |
| 206103_at | 213069_at | 202346_at | 209505_at |
| 201096_s_at | 214439_x_at | 209482_at | 203178_at |
| 209147_s_at | 206375_s_at | 220741_s_at | 213891_s_at |
| 213423_x_at | 202228_s_at | 203148_s_at | 205109_s_at |
| 209921_at | 205752_s_at | 213734_at | 205207_at |
| 201193_at | 201312_s_at | 220342_x_at | 206481_s_at |
| 210886_x_at | 203886_s_at | 203415_at | 201743_at |
| 201941_at | 205952_at | 200606_at | 210495_x_at |
| 214522_x_at | 210198_s_at | 213234_at | 203632_s_at |
| 209228_x_at | 211026_s_at | 208764_s_at | 215193_x_at |
| 208722_s_at | 205251_at | 210018_x_at | 204140_at |
| 218788_s_at | 212463_at | 206790_s_at | 204517_at |
| 203629_s_at | 203695_s_at | 221637_s_at | 212197_x_at |
| 208852_s_at | 219902_at | 210296_s_at | 216215_s_at |
| 207655_s_at | 206022_at | 218328_at | 201744_s_at |
| 200803_s_at | 209090_s_at | 202233_s_at | 209374_s_at |
| 218981_at | 212192_at | 217900_at | 212386_at |
| 217962_at | 33760_at | 205750_at | 202291_s_at |
| 202543_s_at | 210276_s_at | 212085_at | 212239_at |
| 217755_at | 211671_s_at | 202785_at | 202947_s_at |
| 214358_at | 206355_at | | AFFX- |
| 202296_s_at | 208146_s_at | 212685_s_at | HSAC07/X00351_M_at |
| 219920_s_at | 201185_at | 217956_s_at | 204518_s_at |
| 202144_s_at | 216442_x_at | 200044_at | 203477_at |
| 203116_s_at | 203813_s_at | 220980_s_at | 201604_s_at |
| 219521_at | 201234_at | 211497_x_at | 202180_s_at |
| 207362_at | 201858_s_at | 201135_at | 218574_s_at |
| 221610_s_at | 201565_s_at | 202178_at | 221502_at |
| 213713_s_at | 216565_x_at | 221786_at | 214894_x_at |
| 208653_s_at | 212268_at | 218989_x_at | 214771_x_at |
| 201962_s_at | 208335_s_at | 210962_s_at | 201082_s_at |
| 210087_s_at | 218683_at | 212219_at | 221870_at |
| 218647_s_at | 219371_s_at | 208841_s_at | 213519_s_at |
| 219362_at | 210632_s_at | 218652_s_at | 208767_s_at |
| 209903_s_at | 203868_s_at | 202960_s_at | 204151_x_at |
| 213301_x_at | 216235_s_at | 202793_at | 202878_s_at |
| 208843_s_at | 215706_x_at | 208950_s_at | 213901_x_at |
| 203008_x_at | 204855_at | 220080_at | 205364_at |
| 200910_at | 213154_s_at | 205294_at | 203071_at |
| 203213_at | 204687_at | 214281_s_at | 213547_at |
| 213843_x_at | 222146_s_at | 202697_at | 218656_s_at |
| 202406_s_at | 208633_s_at | 211034_s_at | 202644_s_at |
| 218680_x_at | 201995_at | 203124_s_at | 203264_s_at |
| 219061_s_at | 212242_at | 200929_at | 202519_at |
| 203721_s_at | 213135_at | 208800_at | 204993_at |
| 205047_s_at | 213620_s_at | 212688_at | 200771_at |
| 200599_s_at | 205022_s_at | 201523_x_at | 212878_s_at |
| 219762_s_at | 218236_s_at | 214156_at | 209646_x_at |
| 218375_at | 205262_at | 202779_s_at | 203687_at |
| 214005_at | 200611_s_at | 212305_s_at | 212387_at |
| 201284_s_at | 213134_x_at | 201503_at | 212071_s_at |
| 220942_x_at | 209896_s_at | 201790_s_at | 208760_at |
| 200947_s_at | 37408_at | 218357_s_at | 212382_at |
| 204949_at | 205577_at | 201830_s_at | 216033_s_at |
| 204427_s_at | 209197_at | 218928_s_at | 211990_at |
| 213116_at | 210613_s_at | 212536_at | 204730_at |
| 218046_s_at | 202156_s_at | 221539_at | 205782_at |
| 205073_at | 211653_x_at | 200873_s_at | 201445_at |
| 219041_s_at | 204797_s_at | 203201_at | 212148_at |
| 209109_s_at | 211991_s_at | 214472_at | 218031_s_at |
| 206307_s_at | 204260_at | 202539_s_at | 212690_at |
| 200750_s_at | 210762_s_at | 203165_s_at | 213306_at |
| 220189_s_at | 203233_at | 218213_s_at | 209699_x_at |
| 204927_at | 215870_s_at | 211423_s_at | 203887_s_at |
| 218016_s_at | 203068_at | 221827_at | 203604_at |
| 211754_s_at | 205578_at | 213501_at | 204790_at |
| 209796_s_at | 202432_at | 202832_at | 221016_s_at |
| 209873_s_at | 209568_s_at | 204123_at | 202117_at |
| 219060_at | 214577_at | 201004_at | 219228_at |
| 65133_i_at | 213110_s_at | 201931_at | 201648_at |
| 202857_at | 202946_s_at | 210186_s_at | 209379_s_at |
| 201549_x_at | 205120_s_at | 201961_s_at | 213316_at |
| 201791_s_at | 203232_s_at | 202194_at | 207118_s_at |
| 204386_s_at | 204344_s_at | 221688_s_at | 204049_s_at |
| 209326_at | 221730_at | 208799_at | 204640_s_at |
| 202996_at | 212605_s_at | 200875_s_at | 209967_s_at |
| 201821_s_at | 212143_s_at | 218982_s_at | 201721_s_at |
| 209971_x_at | 212457_at | 220094_s_at | 205011_at |
| 209695_at | 202908_at | 200098_s_at | 205824_at |
| 218003_s_at | 212923_s_at | 210739_x_at | 202765_s_at |
| 218112_at | 209312_x_at | 222001_x_at | 203017_s_at |
| 212527_at | 214040_s_at | 201587_s_at | 202207_at |
| 213720_s_at | 213138_at | 201653_at | 202205_at |
| 205449_at | 214608_s_at | 205774_at | 202047_s_at |
| 200037_s_at | 213401_s_at | 203484_at | 209263_x_at |
| 208864_s_at | 208723_at | 201479_at | 202008_s_at |
| 217870_s_at | 204979_s_at | 201341_at | 205348_s_at |
| 217761_at | 203749_s_at | 205244_s_at | 205624_at |
| 208674_x_at | 200838_at | 209773_s_at | 202450_s_at |
| 209872_s_at | 202821_s_at | 218192_at | 200816_s_at |
| 213166_x_at | 203231_s_at | 203918_at | 205478_at |
| 213490_s_at | 217795_s_at | 209104_s_at | 201785_at |
| 218919_at | 201425_at | 213995_at | 218880_at |
| 211778_s_at | 212681_at | 208801_at | 207453_s_at |
| 213132_s_at | 217997_at | 202300_at | 210976_s_at |
| 36936_at | 215146_s_at | 213152_s_at | 200609_s_at |
| 201524_x_at | 212561_at | 65517_at | 217506_at |
| 205661_s_at | 212998_x_at | 217827_s_at | 201696_at |
| 207121_s_at | 209691_s_at | 201074_at | 202643_s_at |
| 213498_at | 210751_s_at | 200055_at | 205805_s_at |
| 217301_x_at | 201666_at | 203126_at | 212503_s_at |
| 53968_at | 209443_at | 201819_at | 211819_s_at |
| 203880_at | 204682_at | 203316_s_at | 212518_at |
| 209739_s_at | 202112_at | 206724_at | 202613_at |
| 201772_at | 211986_at | 201512_s_at | 202422_s_at |
| 201622_at | 204491_at | 208447_s_at | 218892_at |
| 201698_s_at | 221903_s_at | 202787_s_at | 202242_at |
| 219293_s_at | 209582_s_at | 202934_at | 203060_s_at |
| 221962_s_at | 207173_x_at | 217551_at | 205548_s_at |
| 208959_s_at | 205383_s_at | 219869_s_at | 203066_at |
| 202983_at | 203590_at | 214779_s_at | 200839_s_at |
| 201098_at | 208963_x_at | 215091_s_at | 203339_at |
| 209150_s_at | 212494_at | 214167_s_at | 35776_at |
| 202308_at | 201108_s_at | 218163_at | 208609_s_at |
| 219733_s_at | 212549_at | 218732_at | 201795_at |
| 210627_s_at | 208096_s_at | 218427_at | 213075_at |
| 208264_s_at | 210973_s_at | 202712_s_at | 212565_at |
| 214011_s_at | 215306_at | 202799_at | 200985_s_at |
| 212767_at | 202931_x_at | 209522_s_at | 200671_s_at |
| 209545_s_at | 201865_x_at | 201619_at | 203889_at |
| 204332_s_at | 201137_s_at | 213365_at | 213422_s_at |
| 211574_s_at | 222024_s_at | 200820_at | 202856_s_at |
| 219913_s_at | 212851_at | 202299_s_at | 209474_s_at |
| 210907_s_at | 201968_s_at | 209110_s_at | 214055_x_at |
| 201339_s_at | 210202_s_at | 218009_s_at | 202501_at |
| 211762_s_at | 212350_at | 212316_at | 204655_at |
| 222077_s_at | 208634_s_at | 220584_at | 202052_s_at |
| 218681_s_at | 216840_s_at | 205145_s_at | 214767_s_at |
| 218962_s_at | 200653_s_at | 217868_s_at | 219165_at |
| 204333_s_at | 205961_s_at | 210859_x_at | 201311_s_at |
| 218695_at | 207978_s_at | 203272_s_at | 218641_at |
| 218532_s_at | 204550_x_at | 207147_at | 208306_x_at |
| 218045_x_at | 205870_at | 201568_at | 201009_s_at |
| 219053_s_at | 201506_at | 205687_at | 208848_at |
| 208689_s_at | 203185_at | 212194_s_at | 203028_s_at |
| 200889_s_at | 212099_at | 200048_s_at | 202284_s_at |
| 218882_s_at | 210201_x_at | 214315_x_at | 203964_at |
| 209433_s_at | 218902_at | 209180_at | 202950_at |
| 214173_x_at | 201537_s_at | 218834_s_at | 203510_at |
| 217846_at | 210875_s_at | 201953_at | 201020_at |
| 200967_at | 204948_s_at | 217716_s_at | 205933_at |
| 209108_at | 205738_s_at | 211162_x_at | 209737_at |
| 201016_at | 212567_s_at | 221475_s_at | 33850_at |
| 204142_at | 209708_at | 202802_at | 214297_at |
| 217645_at | 209082_s_at | 202095_s_at | 217226_s_at |
| 205107_s_at | 203698_s_at | 208675_s_at | 204670_x_at |
| 215519_x_at | 218804_at | 201659_s_at | 210935_s_at |
| 214857_at | 218376_s_at | 218110_at | 202446_s_at |
| 202381_at | 203828_s_at | 221620_s_at | 217066_s_at |
| 206949_s_at | 212414_s_at | 203235_at | 219416_at |
| 214542_x_at | 201850_at | 208638_at | 209015_s_at |
| 205622_at | 243_g_at | 202670_at | 202598_at |
| 202666_s_at | 219304_s_at | 217772_s_at | 203156_at |
| 210250_x_at | 209501_at | 212202_s_at | 201310_s_at |
| 202886_s_at | 207358_x_at | 218756_s_at | 204134_at |
| 218326_s_at | 200601_at | 205812_s_at | 220108_at |
| 218448_at | 218309_at | 202736_s_at | 216333_x_at |
| 201586_s_at | 215543_s_at | 218321_x_at | 204759_at |
| 201909_at | 207124_s_at | 220721_at | 203662_s_at |
| 207721_x_at | 218667_at | 209175_at | 202803_s_at |
| 203827_at | 207317_s_at | 208951_at | 205960_at |
| 212891_s_at | 212328_at | 218268_at | 218648_at |
| 220768_s_at | 207630_s_at | 210357_s_at | 203661_s_at |
| 211936_at | 204863_s_at | 221797_at | 204310_s_at |
| 212496_s_at | 57715_at | 212828_at | 204000_at |
| 204343_at | 209846_s_at | 205074_at | 204820_s_at |
| 201614_s_at | 218152_at | 50374_at | 201161_s_at |
| 213947_s_at | 222088_s_at | 203576_at | 218084_x_at |
| 213379_at | 201266_at | 221003_s_at | 209454_s_at |
| 214117_s_at | 216944_s_at | 212461_at | 207691_x_at |
| 215812_s_at | 212120_at | 201942_s_at | 220955_x_at |
| 210559_s_at | 55081_at | 205538_at | 209598_at |
| 204922_at | 211974_x_at | 218272_at | 215222_x_at |
| 217785_s_at | 207714_s_at | 213988_s_at | 203794_at |
| 207165_at | 205559_s_at | 203379_at | 217211_at |
| 205875_s_at | 217820_s_at | 208639_x_at | 201566_x_at |
| 205938_at | 209437_s_at | 222231_s_at | 204854_at |
| 201011_at | 206710_s_at | 216338_s_at | 218454_at |
| 209300_s_at | 213015_at | 201816_s_at | 220326_s_at |
| 219874_at | 202208_s_at | 201764_at | 206104_at |
| 212825_at | 213309_at | 209407_s_at | 201169_s_at |
| 221462_x_at | 213249_at | 208436_s_at | 213058_at |
| 217927_at | 222158_s_at | 212740_at | 208070_s_at |
| 217970_s_at | 209786_at | 208826_x_at | 212188_at |
| 208872_s_at | 203585_at | 201629_s_at | 202273_at |
| 214271_x_at | 201718_s_at | 203605_at | 214085_x_at |
| 202737_s_at | 209106_at | 219076_s_at | 212259_s_at |
| 202558_s_at | 215333_x_at | 221691_x_at | 219514_at |
| 204244_s_at | 219985_at | 212175_s_at | 211203_s_at |
| 204290_s_at | 218183_at | 210854_x_at | 205081_at |
| 213687_s_at | 212117_at | 200693_at | 212609_s_at |
| 202211_at | 212792_at | 221041_s_at | 209584_x_at |
| 209998_at | 212158_at | 201521_s_at | 205529_s_at |
| 217748_at | 202951_at | 205355_at | 213170_at |
| 91684_g_at | 49452_at | 201972_at | 212223_at |
| 201263_at | 218284_at | 207563_s_at | 212263_at |
| 201406_at | 202820_at | 213399_x_at | 206071_s_at |
| 203270_at | 214736_s_at | 213897_s_at | 205116_at |
| 200082_s_at | 219221_at | 218567_x_at | 203853_s_at |
| 203360_s_at | 212063_at | 207668_x_at | 202552_s_at |
| 209509_s_at | 206382_s_at | 218270_at | 221816_s_at |
| 212311_at | 213451_x_at | 209142_s_at | 218232_at |
| 220587_s_at | 203151_at | 203926_x_at | 204308_s_at |
| 202932_at | 200694_s_at | 209434_s_at | 204438_at |
| 212739_s_at | 37005_at | 200657_at | 202158_s_at |
| 209100_at | 221884_at | 205980_s_at | 205076_s_at |
| 219048_at | 38671_at | 201576_s_at | 219058_x_at |
| 218241_at | 215000_s_at | 220647_s_at | 219025_at |
| 209864_at | 209787_s_at | 39729_at | 221898_at |
| 212322_at | 204794_at | 201501_s_at | 211944_at |
| 219492_at | 201980_s_at | 210532_s_at | 218472_s_at |
| 212637_s_at | 221881_s_at | 220104_at | 212110_at |
| 202469_s_at | 216594_x_at | 202119_s_at | 202123_s_at |
| 211787_s_at | 209198_s_at | 218512_at | 200758_s_at |
| 205077_s_at | 212937_s_at | 206782_s_at | 219737_s_at |
| 218008_at | 212221_x_at | 204128_s_at | 221565_s_at |
| 209262_s_at | 212080_at | 202813_at | 204341_at |
| 218358_at | 212111_at | 200088_x_at | 218627_at |
| 200715_x_at | 209765_at | 214983_at | 218723_s_at |
| 208828_at | 217833_at | 221580_s_at | 222240_s_at |
| 208905_at | 202172_at | 221984_s_at | 212658_at |
| 206492_at | 203811_s_at | 217791_s_at | 200791_s_at |
| 208985_s_at | 201155_s_at | 201327_s_at | 205100_at |
| 201371_s_at | 202616_s_at | 200961_at | 221527_s_at |
| 204941_s_at | 203501_at | 205329_s_at | 213348_at |
| 201530_x_at | 202497_x_at | 218633_x_at | 221666_s_at |
| 208778_s_at | 203256_at | 201317_s_at | 207838_x_at |
| 214442_s_at | 204834_at | 212953_x_at | 214369_s_at |
| 219517_at | 220975_s_at | 218972_at | 209297_at |
| 202425_x_at | 200788_s_at | 219283_at | 205795_at |
| 202705_at | 203518_at | 203997_at | 204436_at |
| 222212_s_at | 219561_at | 213607_x_at | 202371_at |
| 216958_s_at | 208712_at | 204435_at | 219489_s_at |
| 204228_at | 203685_at | 208967_s_at | 200966_x_at |
| 219732_at | 207761_s_at | 218219_s_at | 209960_at |
| 215300_s_at | 202957_at | 202645_s_at | 204735_at |
| 205512_s_at | 203639_s_at | 213292_s_at | 214812_s_at |
| 204005_s_at | 202861_at | 203942_s_at | 203597_s_at |
| 218684_at | 203787_at | 207439_s_at | 202577_s_at |
| 218481_at | 211998_at | 216640_s_at | 220677_s_at |
| 210386_s_at | 218823_s_at | 204675_at | 211518_s_at |
| 206004_at | 204150_at | 221868_at | 209539_at |
| 209617_s_at | 208030_s_at | 220865_s_at | 202953_at |
| 212623_at | 218651_s_at | 218548_x_at | 202069_s_at |
| 212544_at | 202305_s_at | 201478_s_at | 220272_at |
| 213119_at | 201605_x_at | 208654_s_at | 219229_at |
| 205164_at | 209083_at | 222025_s_at | 201828_x_at |
| 209317_at | 212196_at | 204391_x_at | 202723_s_at |
| 200997_at | 203756_at | 218563_at | 206813_at |
| 208805_at | 60471_at | 201872_s_at | 203986_at |
| 215280_s_at | 208679_s_at | 218741_at | 202508_s_at |
| 207833_s_at | 211654_x_at | 221206_at | 212610_at |
| 202096_s_at | 202048_s_at | 204659_s_at | 210829_s_at |
| 213836_s_at | 204028_s_at | 201463_s_at | 212371_at |
| 218816_at | 212702_s_at | 211036_x_at | 200702_s_at |
| 201023_at | 209702_at | 211061_s_at | 214175_x_at |
| 209323_at | 202734_at | 218503_at | 203404_at |
| 202168_at | 205018_s_at | 218529_at | 209071_s_at |
| 218509_at | 202003_s_at | 220742_s_at | 201930_at |
| 218037_at | 212822_at | 204340_at | 211002_s_at |
| 203133_at | 202362_at | 212053_at | 207233_s_at |
| 203252_at | 211473_s_at | 221253_s_at | 213151_s_at |
| 208756_at | 203340_s_at | 220525_s_at | 200836_s_at |
| 218866_s_at | 213455_at | 214830_at | 202439_s_at |
| 219188_s_at | 219024_at | 220782_x_at | 202561_at |
| 218398_at | 203104_at | 210027_s_at | 218345_at |
| 212340_at | 218128_at | 210667_s_at | 207397_s_at |
| 201584_s_at | 45714_at | 217746_s_at | 212604_at |
| 219223_at | 203909_at | 209714_s_at | 200920_s_at |
| 218440_at | 210605_s_at | 200809_x_at | 201021_s_at |
| 201338_x_at | 208112_x_at | 212995_x_at | 219370_at |
| 218857_s_at | 205648_at | 204825_at | 209203_s_at |
| 213041_s_at | 207966_s_at | 203647_s_at | 201120_s_at |
| 211202_s_at | 212670_at | 202738_s_at | 216236_s_at |
| 219342_at | 212367_at | 201359_at | 200905_x_at |
| 212902_at | 205231_s_at | 217725_x_at | 212758_s_at |
| 208977_x_at | 214721_x_at | 220235_s_at | 209194_at |
| 202614_at | 209365_s_at | 204264_at | 205139_s_at |
| 204545_at | 202910_s_at | 218198_at | 212017_at |
| 201077_s_at | 214725_at | 212826_s_at | 209834_at |
| 211177_s_at | 209546_s_at | 218252_at | 209435_s_at |
| 205084_at | 212119_at | 201113_at | 209321_s_at |
| 218202_x_at | 210628_x_at | 58696_at | 222065_s_at |
| 214855_s_at | 212169_at | 218795_at | 213295_at |
| 206499_s_at | 211031_s_at | 212129_at | 209506_s_at |
| 201490_s_at | 215235_at | 205219_s_at | 43427_at |
| 201376_s_at | 206510_at | 208941_s_at | 202617_s_at |
| 213188_s_at | 218831_s_at | 217797_at | 222221_x_at |
| 208687_x_at | 213395_at | 212015_x_at | 218935_at |
| 211758_x_at | 208611_s_at | 212433_x_at | 203305_at |
| 204025_s_at | 218675_at | 212109_at | 221922_at |
| 209391_at | 205611_at | 204067_at | 210089_s_at |
| 213913_s_at | 221485_at | 213726_x_at | 207069_s_at |
| 212247_at | 209075_s_at | 204967_at | 209039_x_at |
| 204263_s_at | 212294_at | 212330_at | 213603_s_at |
| 207831_x_at | 212660_at | 213017_at | 216100_s_at |
| 204824_at | 217911_s_at | 211558_s_at | 215096_s_at |
| 218320_s_at | 211776_s_at | 217256_x_at | 212409_s_at |
| 203744_at | 213817_at | 221689_s_at | 201336_at |
| 202347_s_at | 202756_s_at | 206723_s_at | 205079_s_at |
| 217964_at | 218127_at | 219809_at | 202522_at |
| 203014_x_at | 212608_s_at | 201177_s_at | 200672_x_at |
| 204212_at | 201022_s_at | 212597_s_at | 202638_s_at |
| 217812_at | 209270_at | 201293_x_at | 212706_at |
| 217007_s_at | 212082_s_at | 218361_at | 203414_at |
| 201415_at | 218425_at | 218764_at | 218634_at |
| 204624_at | 219431_at | 211765_x_at | 220407_s_at |
| 219742_at | 201649_at | 211033_s_at | 1405_i_at |
| 207239_s_at | 200655_s_at | 206527_at | 218660_at |
| 200699_at | 218631_at | 205339_at | 212441_at |
| 204853_at | 36030_at | 200691_s_at | 220634_at |
| 210946_at | 213434_at | 201256_at | 202336_s_at |
| 210594_x_at | 212179_at | 202282_at | 213766_x_at |
| 207348_s_at | 202656_s_at | 201588_at | 200713_s_at |
| 202272_s_at | 204249_s_at | 210192_at | 213925_at |
| 219575_s_at | 202897_at | 212415_at | 202254_at |
| 222206_s_at | 203883_s_at | 220607_x_at | 209324_s_at |
| 220354_at | 209732_at | 204767_s_at | 200951_s_at |
| 201630_s_at | 204045_at | 214831_at | 212829_at |
| 202514_at | 211892_s_at | 320_at | 210840_s_at |
| 204039_at | 202657_s_at | 210434_x_at | 205525_at |
| 208757_at | 219525_at | 208716_s_at | 212408_at |
| 214431_at | 208491_s_at | 212396_s_at | 210702_s_at |
| 65588_at | 201040_at | 218282_at | 202510_s_at |
| 209399_at | 204365_s_at | 203311_s_at | 39582_at |
| 219324_at | 212655_at | 214129_at | 38487_at |
| 202900_s_at | 208740_at | 212508_at | 203508_at |
| 212290_at | 218537_at | 209925_at | 203063_at |
| 213427_at | 220233_at | 217726_at | 209009_at |
| 212127_at | 205280_at | 201489_at | 1294_at |
| 218688_at | 202784_s_at | 200925_at | 202328_s_at |
| 218160_at | 209563_x_at | 202534_x_at | 212798_s_at |
| 209421_at | 219670_at | 219211_at | 203332_s_at |
| 202105_at | 214937_x_at | 219203_at | 213034_at |
| 207871_s_at | 216210_x_at | 211113_s_at | 214719_at |
| 219709_x_at | 209069_s_at | 214737_x_at | 209121_x_at |
| 204266_s_at | 211976_at | 206831_s_at | 204912_at |
| 209014_at | 61734_at | 212416_at | 201090_x_at |
| 213610_s_at | 203503_s_at | 213581_at | 208615_s_at |
| 200046_at | 215059_at | 218305_at | 207172_s_at |
| 214789_x_at | 210001_s_at | 221665_s_at | 211700_s_at |
| 201675_at | 203823_at | 208696_at | 215990_s_at |
| 204295_at | 203281_s_at | 220285_at | 202116_at |
| 201458_s_at | 203726_s_at | 218908_at | 200813_s_at |
| 201682_at | 200984_s_at | 202246_s_at | 202646_s_at |
| 212378_at | 201474_s_at | 210023_s_at | 212504_at |
| 203230_at | 200801_x_at | 210523_at | 219451_at |
| 213223_at | 213261_at | 201322_at | 212855_at |
| 205486_at | 217765_at | 218540_at | 206093_x_at |
| 221654_s_at | 212235_at | 217861_s_at | 203891_s_at |
| 209261_s_at | 213567_at | 219302_s_at | 207571_x_at |
| 211378_x_at | 200712_s_at | 203023_at | 205259_at |
| | AFFX- | 216583_x_at | 205325_at |
| 205246_at | HSAC07/X00351_3_at | 218562_s_at | 32094_at |
| 218725_at | 214687_x_at | 203312_x_at | 203249_at |
| 201385_at | 219563_at | 218590_at | 219496_at |
| 209275_s_at | 210785_s_at | 200081_s_at | 203812_at |
| 205850_s_at | 212917_x_at | 205310_at | 204556_s_at |
| 216895_at | 210401_at | 201548_s_at | 200784_s_at |
| 208214_at | 211000_s_at | 200739_s_at | 32259_at |
| 212661_x_at | 218815_s_at | 208709_s_at | 213646_x_at |
| 219289_at | 212420_at | 218436_at | 44702_at |
| 219428_s_at | 201538_s_at | 204031_s_at | 205153_s_at |
| 203287_at | 204136_at | 33814_at | 201885_s_at |
| 209429_x_at | 201380_at | 208676_s_at | 210073_at |
| 209777_s_at | 221447_s_at | 215947_s_at | 211945_s_at |
| 204247_s_at | 209343_at | 218511_s_at | 220230_s_at |
| 219860_at | 214632_at | 201723_s_at | 213688_at |
| 217720_at | 205082_s_at | 201913_s_at | 211948_x_at |
| 222362_at | 207302_at | 204811_s_at | 213939_s_at |
| 206254_at | 203300_x_at | 209238_at | 207071_s_at |
| 200786_at | 202594_at | 202072_at | 212632_at |
| 219862_s_at | 219305_x_at | 203458_at | 213658_at |
| 200074_s_at | 213327_s_at | 213083_at | 202136_at |
| 209284_s_at | 201502_s_at | 205617_at | 201361_at |
| 218661_at | 206453_s_at | 213009_s_at | 205266_at |
| 210149_s_at | 216205_s_at | 45526_g_at | 218691_s_at |
| 202329_at | 210664_s_at | 212484_at | 221503_s_at |
| 216306_x_at | 208671_at | 200651_at | 204421_s_at |
| 218408_at | 213113_s_at | 215159_s_at | 222111_at |
| 202788_at | 204736_s_at | 207168_s_at | 215051_x_at |
| 221772_s_at | 212157_at | 219786_at | 212958_x_at |
| 218653_at | 221905_at | 218130_at | 204606_at |
| 215482_s_at | 209485_s_at | 221791_s_at | 203369_x_at |
| 219676_at | 220911_s_at | 208968_s_at | 212747_at |
| 200009_at | 212262_at | 209520_s_at | 211458_s_at |
| 201218_at | 219523_s_at | 220966_x_at | 206868_at |
| 222234_s_at | 204294_at | 202190_at | 214909_s_at |
| 219129_s_at | 40016_g_at | 202791_s_at | 208454_s_at |
| 221807_s_at | 220974_x_at | 217724_at | 206757_at |
| 204478_s_at | 213867_x_at | 221826_at | 204192_at |
| 203040_s_at | 210926_at | 204133_at | 203735_x_at |
| 213912_at | 215606_s_at | 201290_at | 214808_at |
| 220174_at | 37022_at | 204027_s_at | 213531_s_at |
| 207396_s_at | 212936_at | 218780_at | 204062_s_at |
| 200068_s_at | 219993_at | 200740_s_at | 202795_x_at |
| 218264_at | 203409_at | 40359_at | 203530_s_at |
| 217930_s_at | 218012_at | 212838_at | 202578_s_at |
| 205709_s_at | 214656_x_at | 200022_at | 221885_at |
| 200734_s_at | 219939_s_at | 218123_at | 219278_at |
| 211978_x_at | 211573_x_at | 201613_s_at | 212938_at |
| 203465_at | 210968_s_at | 203713_s_at | 202174_s_at |
| 221018_s_at | 205088_at | 212769_at | 218062_x_at |
| 218689_at | 204542_at | 201771_at | 203879_at |
| 218829_s_at | 221752_at | 212121_at | 46665_at |
| 209440_at | 219602_s_at | 208822_s_at | 219961_s_at |
| 210005_at | 213386_at | 212269_s_at | 205104_at |
| 209804_at | 211058_x_at | 44065_at | 212759_s_at |
| 208466_at | 209193_at | 219075_at | 212302_at |
| 211271_x_at | 214433_s_at | 208917_x_at | 218032_at |
| 214806_at | 202206_at | 206722_s_at | 203586_s_at |
| 221817_at | 211769_x_at | 213699_s_at | 219770_at |
| 212351_at | 212752_at | 214310_s_at | 209840_s_at |
| 213435_at | 212796_s_at | 213941_x_at | 208981_at |
| 221587_s_at | 213944_x_at | 208009_s_at | 215537_x_at |
| 208369_s_at | 221928_at | 219148_at | 40560_at |
| 202978_s_at | 208206_s_at | 219080_s_at | 205786_s_at |
| 218316_at | 202364_at | 220773_s_at | 203919_at |
| 217903_at | 204174_at | 214481_at | 206972_s_at |
| 219931_s_at | 204683_at | 211052_s_at | 214318_s_at |
| 201758_at | 211994_at | 202433_at | 208617_s_at |
| 203208_s_at | 209901_x_at | 210927_x_at | 213394_at |
| 218817_at | 205479_s_at | 202658_at | 219213_at |
| 208072_s_at | 211997_x_at | 208759_at | 211003_x_at |
| 211658_at | 209606_at | 206066_s_at | 214298_x_at |
| 201095_at | 203499_at | 219851_at | 207053_at |
| 221652_s_at | 219767_s_at | 212436_at | 202590_s_at |
| 218101_s_at | 205398_s_at | 203867_s_at | 205341_at |
| 215023_s_at | 218669_at | 219209_at | 204537_s_at |
| 204169_at | 212299_at | 201097_s_at | 214791_at |
| 218636_s_at | 208982_at | 207262_at | 202022_at |
| 208393_s_at | 202575_at | 202063_s_at | 221656_s_at |
| 203500_at | 205006_s_at | 205761_s_at | 202733_at |
| 202189_x_at | 212639_x_at | 204003_s_at | 48031_r_at |
| 201876_at | 218496_at | 204618_s_at | 212803_at |
| 213189_at | 201183_s_at | 204034_at | 218626_at |
| 213082_s_at | 214449_s_at | 218151_x_at | 201375_s_at |
| 208824_x_at | 203278_s_at | 211972_x_at | 200879_s_at |
| 218199_s_at | 220092_s_at | 203192_at | 204552_at |
| 217127_at | 214177_s_at | 205441_at | 220818_s_at |
| 203573_s_at | 219137_s_at | 217968_at | 209402_s_at |
| 213601_at | 204334_at | 221196_x_at | 211006_s_at |
| 208842_s_at | 203592_s_at | 218226_s_at | 203320_at |
| 202059_s_at | 202564_x_at | 212048_s_at | 212895_s_at |
| 212315_s_at | 212360_at | 202632_at | 210115_at |
| 217740_x_at | 212076_at | 212479_s_at | 203599_s_at |
| 214661_s_at | 220142_at | 202331_at | 202455_at |
| 219562_at | 208869_s_at | 219189_at | 219436_s_at |
| 218070_s_at | 204984_at | 200057_s_at | 212468_at |
| 204798_at | 222073_at | 217910_x_at | 200066_at |
| 213762_x_at | 218820_at | 218598_at | 204462_s_at |
| 217961_at | 201752_s_at | 219429_at | 205112_at |
| 213708_s_at | 215493_x_at | 218735_s_at | 218215_s_at |
| 218565_at | 213326_at | 218766_s_at | 205902_at |
| 202159_at | 204633_s_at | 204883_s_at | 201379_s_at |
| 208856_x_at | 202998_s_at | 203314_at | 213203_at |
| 37831_at | 211072_x_at | 201330_at | 37384_at |
| 217466_x_at | 200051_at | 201716_at | 210794_s_at |
| 33307_at | 210102_at | 203719_at | 202262_x_at |
| 207812_s_at | 209867_s_at | 211392_s_at | 218373_at |
| 212118_at | 208786_s_at | 205324_s_at | 209688_s_at |
| 214537_at | 213095_x_at | 203022_at | 209721_s_at |
| 35201_at | 213417_at | 221891_x_at | 206649_s_at |
| 201349_at | 218870_at | 219723_x_at | 213940_s_at |
| 205634_x_at | 203047_at | 207654_x_at | 213513_x_at |
| 203677_s_at | 215346_at | 203869_at | 208859_s_at |
| 201886_at | 222379_at | 221572_s_at | 218266_s_at |
| 204962_s_at | 204882_at | 209145_s_at | 204198_s_at |
| 204488_at | 203894_at | 203358_s_at | 211043_s_at |
| 37950_at | 209251_x_at | 206919_at | 40472_at |
| 221818_at | 202039_at | 203947_at | 205240_at |
| 200627_at | 204989_s_at | 206109_at | 202921_s_at |
| 201459_at | 221473_x_at | 201709_s_at | 207895_at |
| 201391_at | 202652_at | 202217_at | 202806_at |
| 218868_at | 208018_s_at | 221777_at | 217946_s_at |
| 212395_s_at | 202579_x_at | 200843_s_at | 221484_at |
| 210761_s_at | 203944_x_at | 209053_s_at | 218997_at |
| 201420_s_at | 201460_at | 216397_s_at | 213260_at |
| 218289_s_at | 202916_s_at | 219033_at | 211701_s_at |
| 216652_s_at | 203456_at | 211720_x_at | 203733_at |
| 209188_x_at | 213630_at | 219176_at | 213644_at |
| 32209_at | 208868_s_at | 218797_s_at | 210574_s_at |
| 204117_at | 213030_s_at | 218455_at | 214179_s_at |
| 219050_s_at | 204428_s_at | 215982_s_at | 52651_at |
| 213885_at | 213556_at | 205909_at | 202783_at |
| 202488_s_at | 206284_x_at | 212871_at | 200759_x_at |
| 204809_at | 203167_at | 216985_s_at | 221779_at |
| 204695_at | 202858_at | 220661_s_at | 219457_s_at |
| 219797_at | 208964_s_at | 209592_s_at | 211668_s_at |
| 204108_at | 222199_s_at | 218953_s_at | 209866_s_at |
| 205429_s_at | 208158_s_at | 206194_at | 214181_x_at |
| 204423_at | 213698_at | 218855_at | 203197_s_at |
| 201033_x_at | 217362_x_at | 213237_at | 221991_at |
| 212719_at | 212715_s_at | 213115_at | 203674_at |
| 209618_at | 219520_s_at | 203160_s_at | 53720_at |
| 205963_s_at | 202530_at | 212486_s_at | 207629_s_at |
| 218874_s_at | 210224_at | 205111_s_at | 217904_s_at |
| 204954_s_at | 212642_s_at | 209831_x_at | 40446_at |
| 221800_s_at | 213876_x_at | 215311_at | 218310_at |
| 206173_x_at | 222171_s_at | 52975_at | 204763_s_at |
| 219154_at | 202092_s_at | 205447_s_at | 212227_x_at |
| 203046_s_at | 206178_at | 212818_s_at | 211750_x_at |
| 218988_at | 204044_at | 206637_at | 205111_s_at |
| 204561_x_at | 214853_s_at | 204636_at | 211780_x_at |
| 204903_x_at | 208741_at | 210140_at | 215253_s_at |
| 50965_at | 37152_at | 204502_at | 206050_s_at |
| 218159_at | 214285_at | 205543_at | 210692_s_at |
| 217839_at | 214823_at | 219838_at | 219620_x_at |
| 209830_s_at | 219628_at | 219801_at | 219243_at |
| 43977_at | 209726_at | 210408_s_at | 203062_s_at |
| 208648_at | 201934_at | 211871_x_at | 200886_s_at |
| 65086_at | 206009_at | 219815_at | 206122_at |
| 210410_s_at | 213252_at | 214078_at | 202640_s_at |
| 213608_s_at | 36829_at | 204221_x_at | 212550_at |
| 219828_at | 209204_at | 209827_s_at | 205405_at |
| 216086_at | 202894_at | 217965_s_at | 204513_s_at |
| 201759_at | 212695_at | 207375_s_at | 220027_s_at |
| 221591_s_at | 212427_at | 213804_at | 204303_s_at |
| 204717_s_at | 213270_at | 207436_x_at | 218844_at |
| 221222_s_at | 220937_s_at | 212550_at | 208103_s_at |
| 221738_at | 218337_at | 219821_s_at | 221506_s_at |
| 212429_s_at | 219367_s_at | 209716_at | 200673_at |
| 208903_at | 207984_s_at | 213533_at | 221021_s_at |
| 202945_at | 203666_at | 219970_at | 209877_at |
| 204578_at | 212134_at | 209603_at | 221552_at |
| 204366_s_at | 205528_s_at | 53991_at | 212130_x_at |
| 222081_at | 212045_at | 202744_at | 218950_at |
| 206688_s_at | 217025_s_at | 203217_s_at | 212447_at |
| 220631_at | 203045_at | 205192_at | 207971_s_at |
| 220144_s_at | 222217_s_at | 207614_s_at | 203757_s_at |
| 203483_at | 201471_s_at | 207457_s_at | 31845_at |
| 221886_at | 202098_s_at | 204437_s_at | 208858_s_at |
| 203010_at | 208325_s_at | 203187_at | 212024_x_at |
| 217452_s_at | 205121_at | 220452_x_at | 205270_s_at |
| 214617_at | 205918_at | 64942_at | 204502_at |
| 202663_at | 208174_x_at | 203734_at | 205632_s_at |
| 211256_x_at | 206518_s_at | 204879_at | 211809_x_at |
| 213906_at | 215767_at | 219390_at | 209716_at |
| 220246_at | 53991_at | 214033_at | 217721_at |
| 204982_at | 211316_x_at | 215506_s_at | 213906_at |
| 218029_at | 203514_at | 208213_s_at | 210648_x_at |
| 204504_s_at | 210880_s_at | 212823_s_at | 212516_at |
| 221832_s_at | 204627_s_at | 205112_at | 202191_s_at |
| 219738_s_at | 213066_at | 203598_s_at | 209534_x_at |
| 219464_at | 218424_s_at | 35846_at | 204038_s_at |
| 209243_s_at | 205192_at | 211843_x_at | 218999_at |
| 206403_at | 211871_x_at | 202530_at | 204747_at |
| 200015_s_at | 219195_at | 204552_at | 64942_at |
| 206009_at | 221090_s_at | 205121_at | 209789_at |
| 206178_at | 201184_s_at | 210692_s_at | 208044_s_at |
| 203798_s_at | 209320_at | 200066_at | 211401_s_at |
| 203741_s_at | 200015_s_at | 218805_at | 219815_at |
| 211072_x_at | 215439_x_at | 219213_at | 203734_at |
| 221753_at | 35846_at | 212639_x_at | 210140_at |
| 213509_x_at | 205001_s_at | 204513_s_at | 206682_at |
| 211194_s_at | 214604_at | 205255_x_at | 202828_s_at |
| 212130_x_at | 208213_s_at | 218266_s_at | 207375_s_at |
| 216017_s_at | 204043_at | 206050_s_at | 205447_s_at |
| 203348_s_at | 40420_at | 218997_at | 213012_at |
| 212227_x_at | 207747_s_at | 201515_s_at | 209401_s_at |
| 209789_at | 203598_s_at | 212926_at | 212486_s_at |
| 217914_at | 221551_x_at | 204642_at | 212672_at |
| 40472_at | 207643_s_at | 213030_s_at | 218497_s_at |
| 37152_at | 217965_s_at | 213066_at | 219677_at |
| 217721_at | 213467_at | 203045_at | 219821_s_at |
| 209940_at | 214436_at | 214118_x_at | 212823_s_at |
| 210882_s_at | 209243_s_at | 205760_s_at | 217220_at |
| 220027_s_at | 219593_at | 214285_at | 219801_at |
| 204043_at | 201515_s_at | 203167_at | 219616_at |
| 217220_at | 207988_s_at | 204038_s_at | 204504_s_at |
| 211330_s_at | 214078_at | 218677_at | 212970_at |
| 52837_at | 202410_x_at | 202410_x_at | 214036_at |
| 221044_s_at | 211366_x_at | 40560_at | 213266_at |
| 221656_s_at | 221699_s_at | 218950_at | 218805_at |
| 211809_x_at | 205575_at | 205240_at | 207034_s_at |
| 214995_s_at | 211729_x_at | 211780_x_at | 35617_at |
| 211325_x_at | 209970_x_at | 213932_x_at | 219039_at |
| 219114_at | 219114_at | 219529_at | 211256_x_at |
| 203197_s_at | 207614_s_at | 213922_at | 212836_at |
| 210079_x_at | 207457_s_at | 203456_at | 216705_s_at |
| 212079_s_at | 221901_at | 219616_at | 52837_at |
| 37384_at | 213269_at | 221779_at | 221753_at |
| 221552_at | 221883_at | 214853_s_at | 217691_x_at |
| 207053_at | 219944_at | 208325_s_at | 203187_at |
| 212134_at | 210079_x_at | 219195_at | 202663_at |
| 221699_s_at | 204982_at | 203069_at | 212818_s_at |
| 220016_at | 336_at | 215439_x_at | 219390_at |
| 206191_at | 213804_at | 202092_s_at | 32502_at |
| 210794_s_at | 216017_s_at | 206087_x_at | 203904_x_at |
| 219768_at | 212400_at | 204627_s_at | 635_s_at |
| 52651_at | 218775_s_at | 200886_s_at | 205543_at |
| 221551_x_at | 219970_at | 205159_at | 203490_at |
| 218775_s_at | 218029_at | 209688_s_at | 208460_at |
| 36829_at | 204642_at | 203592_s_at | 210882_s_at |
| 210347_s_at | 213530_at | 213644_at | 220452_x_at |
| 211058_x_at | 221234_s_at | 203047_at | 201270_x_at |
| 209877_at | 205277_at | 218807_at | 213885_at |
| 220937_s_at | 203488_at | 205405_at | 50965_at |
| 207747_s_at | 205599_at | 203757_s_at | 209171_at |
| 209320_at | 48117_at | 207984_s_at | 212280_x_at |
| 202098_s_at | 203348_s_at | 204047_s_at | 209618_at |
| 203530_s_at | 38149_at | 204428_s_at | 221052_at |
| 204747_at | 212748_at | 217312_s_at | 215734_at |
| 201934_at | 218429_s_at | 202652_at | 204234_s_at |
| 209721_s_at | 202256_at | 218802_at | 208842_s_at |
| 218310_at | 221832_s_at | 212695_at | 219148_at |
| 217608_at | 210144_at | 206033_s_at | 205429_s_at |
| 213269_at | 214617_at | 204044_at | 214806_at |
| 31845_at | 45749_at | 222217_s_at | 203046_s_at |
| 208103_s_at | 205911_at | 202590_s_at | 207654_x_at |
| 213270_at | 210607_at | 220142_at | 221036_s_at |
| 217993_s_at | 205560_at | 213646_x_at | 218766_s_at |
| 217904_s_at | 220399_at | 204763_s_at | 211801_x_at |
| 207988_s_at | 220144_s_at | 219767_s_at | 208393_s_at |
| 211892_s_at | 206688_s_at | 213100_at | 202059_s_at |
| 213630_at | 213679_at | 219684_at | 201977_s_at |
| 211401_s_at | 207018_s_at | 212076_at | 212479_s_at |
| 211668_s_at | 209910_at | 204174_at | 201420_s_at |
| 207971_s_at | 212790_x_at | 204589_at | 219238_at |
| 213467_at | 34221_at | 203666_at | 217910_x_at |
| 205104_at | 217598_at | 202191_s_at | 209145_s_at |
| 221234_s_at | 219154_at | 205528_s_at | 205243_at |
| 205008_s_at | 210410_s_at | 204177_s_at | 212436_at |
| 215767_at | 209745_at | 201294_s_at | 204883_s_at |
| 208018_s_at | 208903_at | 209257_s_at | 213685_at |
| 210702_s_at | 214210_at | 61734_at | 212719_at |
| 210736_x_at | 213608_s_at | 201090_x_at | 220661_s_at |
| 212360_at | 43977_at | 209841_s_at | 217930_s_at |
| 209534_x_at | 202945_at | 204633_s_at | 218868_at |
| 212803_at | 205909_at | 216187_x_at | 207396_s_at |
| 205786_s_at | 209672_s_at | 209308_s_at | 205850_s_at |
| 209867_s_at | 221550_at | 204556_s_at | 218558_s_at |
| 220071_x_at | 213393_at | 206122_at | 213237_at |
| 218424_s_at | 205432_at | 201183_s_at | 202791_s_at |
| 40446_at | 218953_s_at | 219134_at | 221818_at |
| 221885_at | 221738_at | 204736_s_at | 219538_at |
| 212373_at | 207059_at | 210785_s_at | 203208_s_at |
| 214036_at | 211720_x_at | 219628_at | 218874_s_at |
| 212427_at | 218159_at | 205902_at | 208009_s_at |
| 214909_s_at | 219635_at | 203278_s_at | 204809_at |
| 219602_s_at | 213115_at | 202831_at | 214481_at |
| 40837_at | 218146_at | 53720_at | 209195_s_at |
| 212235_at | 219723_x_at | 213260_at | 212395_s_at |
| 215493_x_at | 208648_at | 215411_s_at | 213063_at |
| 214436_at | 208569_at | 221795_at | 208955_at |
| 209866_s_at | 33307_at | 200813_s_at | 218562_s_at |
| 211366_x_at | 204402_at | 219243_at | 204476_s_at |
| 212299_at | 222018_at | 203879_at | 213223_at |
| 218373_at | 218598_at | 203944_x_at | 204798_at |
| 220634_at | 213601_at | 219563_at | 213009_s_at |
| 203586_s_at | 204903_x_at | 212706_at | 219209_at |
| 200697_at | 201033_x_at | 202646_s_at | 208856_x_at |
| 205632_s_at | 203947_at | 206032_at | 217740_x_at |
| 212468_at | 216652_s_at | 204882_at | 203790_s_at |
| 204062_s_at | 219033_at | 209726_at | 208923_at |
| 205453_at | 202632_at | 203369_x_at | 211378_x_at |
| 202783_at | 44065_at | 220818_s_at | 204003_s_at |
| 208158_s_at | 209188_x_at | 211006_s_at | 221018_s_at |
| 202022_at | 221508_at | 205325_at | 39966_at |
| 204063_s_at | 220773_s_at | 211316_x_at | 219129_s_at |
| 207895_at | 215215_s_at | 212629_s_at | 203040_s_at |
| 214298_x_at | 202063_s_at | 202522_at | 206919_at |
| 219436_s_at | 209440_at | 219961_s_at | 213708_s_at |
| 206972_s_at | 204169_at | 218691_s_at | 203287_at |
| 202733_at | 204423_at | 208869_s_at | 208778_s_at |
| 203812_at | 218199_s_at | 212796_s_at | 218988_at |
| 213095_x_at | 208696_at | 210926_at | 211765_x_at |
| 215606_s_at | 218797_s_at | 205525_at | 201709_s_at |
| 202578_s_at | 218249_at | 221484_at | 210192_at |
| 214725_at | 208822_s_at | 203853_s_at | 212127_at |
| 211701_s_at | 206587_at | 202206_at | 213083_at |
| 39582_at | 203800_s_at | 209901_x_at | 208968_s_at |
| 204334_at | 213189_at | 221991_at | 211658_at |
| 203662_s_at | 218511_s_at | 202254_at | 201771_at |
| 208206_s_at | 218316_at | 213394_at | 209777_s_at |
| 38487_at | 217961_at | 211657_at | 212121_at |
| 212715_s_at | 202031_s_at | 221901_at | 204008_at |
| 219545_at | 202331_at | 219939_s_at | 212342_at |
| 208616_s_at | 210005_at | 202116_at | 203500_at |
| 209970_x_at | 37831_at | 214791_at | 204853_at |
| 200916_at | 215482_s_at | 204198_s_at | 204618_s_at |
| 203320_at | 211972_x_at | 203894_at | 222362_at |
| 219520_s_at | 220966_x_at | 201146_at | 217256_x_at |
| 212157_at | 206109_at | 222171_s_at | 201489_at |
| 210073_at | 208985_s_at | 214629_x_at | 221156_x_at |
| 213203_at | 203677_s_at | 201361_at | 205928_at |
| 221473_x_at | 211212_s_at | 203661_s_at | 211113_s_at |
| 202795_x_at | 211978_x_at | 203037_s_at | 34764_at |
| 207571_x_at | 219080_s_at | 219523_s_at | 201723_s_at |
| 202998_s_at | 219742_at | 209332_s_at | 219562_at |
| 203797_at | 207262_at | 203919_at | 204353_s_at |
| 203508_at | 203573_s_at | 220677_s_at | 212155_at |
| 203074_at | 219075_at | 205231_s_at | 219066_at |
| 200673_at | 213941_x_at | 48031_r_at | 204050_s_at |
| 203599_s_at | 209925_at | 201380_at | 218911_at |
| 218032_at | 202713_s_at | 214177_s_at | 202306_at |
| 215990_s_at | 209429_x_at | 209402_s_at | 200651_at |
| 213590_at | 218392_x_at | 202000_at | 218289_s_at |
| 219597_s_at | 204488_at | 219014_at | 218725_at |
| 37022_at | 214864_s_at | 220108_at | 213435_at |
| 222073_at | 201758_at | 210401_at | 218688_at |
| 214052_x_at | 216945_x_at | 202613_at | 201293_x_at |
| 203249_at | 221791_s_at | 32094_at | 208596_s_at |
| 205398_s_at | 219097_x_at | 205611_at | 207168_s_at |
| 213271_s_at | 208369_s_at | 211031_s_at | 203816_at |
| 221928_at | 218160_at | 204421_s_at | 212661_x_at |
| 213556_at | 200739_s_at | 213217_at | 203330_s_at |
| 222221_x_at | 209284_s_at | 202328_s_at | 40359_at |
| 204683_at | 212015_x_at | 213478_at | 202272_s_at |
| 211368_s_at | 200734_s_at | 207071_s_at | 220318_at |
| 204912_at | 215947_s_at | 205823_at | 200068_s_at |
| 205479_s_at | 202105_at | 213113_s_at | 200022_at |
| 46665_at | 208466_at | 202965_s_at | 218512_at |
| 44702_at | 201113_at | 212409_s_at | 218540_at |
| 202449_s_at | 210761_s_at | 211726_s_at | 218070_s_at |
| 208786_s_at | 216380_x_at | 210089_s_at | 208687_x_at |
| 32259_at | 219223_at | 218487_at | 205339_at |
| 208112_x_at | 208941_s_at | 209703_x_at | 218817_at |
| 204462_s_at | 203713_s_at | 208964_s_at | 205371_s_at |
| 210224_at | 58696_at | 213326_at | 219321_at |
| 203185_at | 204247_s_at | 204606_at | 222206_s_at |
| 216594_x_at | 205634_x_at | 215059_at | 202487_s_at |
| 200788_s_at | 218741_at | AFFX- | |
| 218669_at | 201209_at | HSAC07/X00351_3_at | 201913_s_at |
| 218634_at | 202282_at | 216100_s_at | 221196_x_at |
| 214604_at | 219463_at | 209198_s_at | 208072_s_at |
| 218820_at | 217968_at | 220092_s_at | 218653_at |
| 221905_at | 213699_s_at | 218935_at | 209391_at |
| 202579_x_at | 221807_s_at | 204150_at | 201239_s_at |
| 203063_at | 208759_at | 209015_s_at | 209421_at |
| 215051_x_at | 200657_at | 212855_at | 213427_at |
| 211675_s_at | 217944_at | 213531_s_at | 216895_at |
| 208491_s_at | 218069_at | 213295_at | 200809_x_at |
| 201474_s_at | 207871_s_at | 209474_s_at | 204378_at |
| 200801_x_at | 222234_s_at | 205116_at | 219255_x_at |
| 217802_s_at | 209238_at | 213513_x_at | 203437_at |
| 213567_at | 212861_at | 219496_at | 214271_x_at |
| 202897_at | 218123_at | 208859_s_at | 220603_s_at |
| 204546_at | 222025_s_at | 201718_s_at | 219203_at |
| 212326_at | 219289_at | 220974_x_at | 201512_s_at |
| 212262_at | 217976_s_at | 207691_x_at | 201672_s_at |
| 209606_at | 209262_s_at | 204537_s_at | 204360_s_at |
| 213867_x_at | 213912_at | 213925_at | 217791_s_at |
| 203650_at | 212351_at | 205259_at | 205441_at |
| 208454_s_at | 218101_s_at | 218815_s_at | 218436_at |
| 204341_at | 215023_s_at | 211819_s_at | 202811_at |
| 203811_s_at | 206556_at | 36030_at | 218636_s_at |
| 200713_s_at | 211098_x_at | 212177_at | 209804_at |
| 218472_s_at | 207156_at | 201375_s_at | 202900_s_at |
| 214808_at | 221696_s_at | 212371_at | 206004_at |
| 222008_at | 202322_s_at | 204134_at | 204295_at |
| 215313_x_at | 206492_at | 211000_s_at | 201629_s_at |
| 201537_s_at | 202488_s_at | 215346_at | 202514_at |
| 205088_at | 212433_x_at | 203482_at | 208659_at |
| 219431_at | 91684_g_at | 200984_s_at | 219676_at |
| 201980_s_at | 211036_x_at | 204136_at | 206831_s_at |
| 209602_s_at | 210768_x_at | 205315_s_at | 201077_s_at |
| 221485_at | 214442_s_at | 218731_s_at | 209617_s_at |
| 204436_at | 218834_s_at | 221503_s_at | 205761_s_at |
| 211769_x_at | 221826_at | 209598_at | 211558_s_at |
| 209960_at | 215300_s_at | 203499_at | 219786_at |
| 219764_at | 204478_s_at | 210875_s_at | 206533_at |
| 218012_at | 202433_at | 218425_at | 201614_s_at |
| 210840_s_at | 201886_at | 218128_at | 201385_at |
| 216210_x_at | 204034_at | 212082_s_at | 207833_s_at |
| 209039_x_at | 210594_x_at | 218651_s_at | 205617_at |
| 206243_at | 207827_x_at | 202910_s_at | 218209_s_at |
| 213766_x_at | 208107_s_at | 200676_s_at | 36475_at |
| 201403_s_at | 203252_at | 209840_s_at | 212740_at |
| 217109_at | 210023_s_at | 210880_s_at | 218252_at |
| 202561_at | 206066_s_at | 202136_at | 203738_at |
| 213034_at | 203569_s_at | 202048_s_at | 217958_at |
| 33850_at | 213188_s_at | 212504_at | 200740_s_at |
| 213817_at | 208821_at | 43427_at | 214831_at |
| 212188_at | 201613_s_at | 209765_at | 213610_s_at |
| 207317_s_at | 201588_at | 214297_at | 219307_at |
| 60471_at | 219709_x_at | 217066_s_at | 200691_s_at |
| 202510_s_at | 203926_x_at | 200758_s_at | 209317_at |
| 202439_s_at | 219428_s_at | 201785_at | 206722_s_at |
| 222199_s_at | 220607_x_at | 212798_s_at | 209433_s_at |
| 213658_at | 200875_s_at | 221875_x_at | 220934_s_at |
| 205795_at | 220174_at | 209570_s_at | 201095_at |
| 209719_x_at | 220647_s_at | 200900_s_at | 205512_s_at |
| 208617_s_at | 202190_at | 213940_s_at | 219860_at |
| 213434_at | 218180_s_at | 221805_at | 219575_s_at |
| 205006_s_at | 203682_s_at | 212758_s_at | 203458_at |
| 221447_s_at | 218509_at | 220911_s_at | 204088_at |
| 209203_s_at | 218133_s_at | 204222_s_at | 218780_at |
| 212408_at | 202852_s_at | 218844_at | 204675_at |
| 203535_at | 217249_x_at | 207302_at | 210927_x_at |
| 204308_s_at | 219771_at | 209539_at | 202705_at |
| 202856_s_at | 214011_s_at | 219058_x_at | 218198_at |
| 220230_s_at | 200088_x_at | 205139_s_at | 203925_at |
| 210829_s_at | 201175_at | 204365_s_at | 211061_s_at |
| 220115_s_at | 218481_at | 202803_s_at | 200925_at |
| 213939_s_at | 203154_s_at | 212658_at | 221206_at |
| 211776_s_at | 209323_at | 210561_s_at | 207563_s_at |
| 206868_at | 201478_s_at | 202362_at | 205140_at |
| 205005_s_at | 219324_at | 205551_at | 208805_at |
| 204045_at | 201682_at | 218062_x_at | 207831_x_at |
| 203409_at | 208405_s_at | 218127_at | 219188_s_at |
| 212196_at | 202604_x_at | 205267_at | 200750_s_at |
| 201885_s_at | 206527_at | 220955_x_at | 214789_x_at |
| 210976_s_at | 203621_at | 202861_at | 220334_at |
| 204542_at | 217835_x_at | 209009_at | 219874_at |
| 243_g_at | 217861_s_at | 220272_at | 204862_s_at |
| 214812_s_at | 222001_x_at | 219451_at | 203312_x_at |
| 209435_s_at | 217720_at | 203909_at | 221797_at |
| 219514_at | 203014_x_at | 211653_x_at | 206782_s_at |
| 212792_at | 218008_at | 207714_s_at | 204212_at |
| 217211_at | 212426_s_at | 204989_s_at | 204228_at |
| 218345_at | 217797_at | 219670_at | 221253_s_at |
| 207069_s_at | 211202_s_at | 202594_at | 208756_at |
| 204215_at | 204025_s_at | 1294_at | 202671_s_at |
| 203567_s_at | 219302_s_at | 212822_at | 212902_at |
| 209083_at | 217929_s_at | 212169_at | 218005_at |
| 203787_at | 219851_at | 38671_at | 207439_s_at |
| 207838_x_at | 221817_at | 201021_s_at | 220865_s_at |
| 203340_s_at | 201338_x_at | 218332_at | 202697_at |
| 212567_s_at | 204811_s_at | 212294_at | 210409_at |
| 206854_s_at | 209434_s_at | 201828_x_at | 212508_at |
| 201506_at | 201256_at | 205738_s_at | 204244_s_at |
| 211203_s_at | 213913_s_at | 204249_s_at | 221654_s_at |
| 209297_at | 218756_s_at | 207705_s_at | 217772_s_at |
| 209699_x_at | 212416_at | 202656_s_at | 203152_at |
| 213603_s_at | 210532_s_at | 215222_x_at | 219809_at |
| 1405_i_at | 207147_at | 209702_at | 212597_s_at |
| 208096_s_at | 202329_at | 203726_s_at | 218270_at |
| 213395_at | 212006_at | 204151_x_at | 202120_x_at |
| 202617_s_at | 216295_s_at | 201649_at | 201371_s_at |
| 205076_s_at | 214156_at | 221527_s_at | 212622_at |
| 215867_x_at | 218788_s_at | 203503_s_at | 210386_s_at |
| 218660_at | 209399_at | 214937_x_at | 209817_at |
| 204834_at | 220587_s_at | 212565_at | 218684_at |
| 201336_at | 217785_s_at | 213698_at | 213307_at |
| 209563_x_at | 218529_at | 209194_at | 201909_at |
| 201287_s_at | 202788_at | 203151_at | 213947_s_at |
| 209732_at | 205190_at | 207397_s_at | 218264_at |
| 213261_at | 219293_s_at | 212441_at | 200997_at |
| 201795_at | 212637_s_at | 202657_s_at | 221689_s_at |
| 206382_s_at | 221868_at | 202378_s_at | 209104_s_at |
| 207233_s_at | 204167_at | 201155_s_at | 214983_at |
| 214369_s_at | 206993_at | 221730_at | 218320_s_at |
| 219305_x_at | 212995_x_at | 219025_at | 213607_x_at |
| 213151_s_at | 220525_s_at | 209454_s_at | 220495_s_at |
| 205082_s_at | 218398_at | 202158_s_at | 214006_s_at |
| 207453_s_at | 210250_x_at | 211997_x_at | 204161_s_at |
| 206071_s_at | 221597_s_at | 213386_at | 220235_s_at |
| 201022_s_at | 217812_at | 202784_s_at | 202658_at |
| 205079_s_at | 218689_at | 204682_at | 203744_at |
| 205153_s_at | 220285_at | 202273_at | 218361_at |
| 203883_s_at | 219517_at | 211473_s_at | 205774_at |
| 209834_at | 203987_at | 212063_at | 205770_at |
| 201108_s_at | 217932_at | 211458_s_at | 208906_at |
| 212660_at | 218764_at | 217820_s_at | 210058_at |
| 204048_s_at | 217809_at | 209569_x_at | 218882_s_at |
| 204482_at | 212129_at | 202820_at | 33814_at |
| 202478_at | 204263_s_at | 202756_s_at | 202802_at |
| 214656_x_at | 218795_at | 204438_at | 200620_at |
| 219416_at | 201349_at | 218631_at | 203647_s_at |
| 218084_x_at | 219733_s_at | 203698_s_at | 213292_s_at |
| 206600_s_at | 211787_s_at | 207124_s_at | 220104_at |
| 218648_at | 202813_at | 220326_s_at | 209100_at |
| 203794_at | 35671_at | 219229_at | 209407_s_at |
| 212223_at | 222231_s_at | 202501_at | 213897_s_at |
| 203332_s_at | 218358_at | 212420_at | 219053_s_at |
| 208030_s_at | 200693_at | 202577_s_at | 202144_s_at |
| 209365_s_at | 201530_x_at | 213455_at | 219211_at |
| 205559_s_at | 207165_at | 214577_at | 218772_x_at |
| 202957_at | 221539_at | 200655_s_at | 202799_at |
| 212457_at | 201458_s_at | 218368_s_at | 201456_s_at |
| 202552_s_at | 202347_s_at | 49452_at | 217827_s_at |
| 203828_s_at | 214751_at | 218641_at | 217898_at |
| 214624_at | 202645_s_at | 213138_at | 204067_at |
| 212702_s_at | 212415_at | 204948_s_at | 201576_s_at |
| 200791_s_at | 210854_x_at | 211700_s_at | 201415_at |
| 202723_s_at | 214173_x_at | 202508_s_at | 209014_at |
| 203756_at | 201317_s_at | 202003_s_at | 212544_at |
| 214211_at | 221475_s_at | 205100_at | 221665_s_at |
| 203104_at | 201406_at | 212080_at | 203942_s_at |
| 221565_s_at | 204435_at | 212367_at | 212519_at |
| 203281_s_at | 218341_at | 214460_at | 204624_at |
| 211518_s_at | 208613_s_at | 208763_s_at | 218282_at |
| 216944_s_at | 218440_at | 212259_s_at | 217746_s_at |
| 205870_at | 222212_s_at | 208070_s_at | 202168_at |
| 218309_at | 218427_at | 220975_s_at | 50374_at |
| 202371_at | 203351_s_at | 219561_at | 206949_s_at |
| 218831_s_at | 201023_at | 204670_x_at | 218202_x_at |
| 209321_s_at | 220354_at | 35776_at | 217748_at |
| 200920_s_at | 218866_s_at | 212917_x_at | 205661_s_at |
| 208671_at | 217726_at | 200694_s_at | 219060_at |
| 202259_s_at | 218219_s_at | 209582_s_at | 218111_s_at |
| 216840_s_at | 218695_at | 219525_at | 200037_s_at |
| 210605_s_at | 201587_s_at | 205648_at | 213498_at |
| 212263_at | 202025_x_at | 204979_s_at | 202670_at |
| 204797_s_at | 221462_x_at | 205207_at | 200082_s_at |
| 205529_s_at | 212825_at | 204011_at | 219492_at |
| 215096_s_at | 201501_s_at | 209081_s_at | 217716_s_at |
| 200884_at | 201003_x_at | 220952_s_at | 212461_at |
| 216894_x_at | 207722_s_at | 209437_s_at | 207121_s_at |
| 212117_at | 202767_at | 204854_at | 202959_at |
| 209485_s_at | 202320_at | 204000_at | 206723_s_at |
| 213737_x_at | 205161_s_at | 212851_at | 201341_at |
| 202616_s_at | 218163_at | 206458_s_at | 217200_x_at |
| 210762_s_at | 209130_at | 206375_s_at | 208757_at |
| 214823_at | 202738_s_at | 210201_x_at | 219215_s_at |
| 214736_s_at | 209479_at | 202446_s_at | 204266_s_at |
| 209075_s_at | 203270_at | 209506_s_at | 36936_at |
| 209307_at | 209233_at | 213058_at | 210523_at |
| 202575_at | 218037_at | 204820_s_at | 219521_at |
| 200702_s_at | 201074_at | 210102_at | 207668_x_at |
| 200609_s_at | 208270_s_at | 212494_at | 204066_s_at |
| 208679_s_at | 210357_s_at | 205824_at | 204290_s_at |
| 201040_at | 202787_s_at | 218183_at | 218491_s_at |
| 218627_at | 220768_s_at | 202734_at | 208674_x_at |
| 208712_at | 39729_at | 218284_at | 209509_s_at |
| 215000_s_at | 202614_at | 202047_s_at | 212739_s_at |
| 213422_s_at | 200715_x_at | 210973_s_at | 203213_at |
| 209069_s_at | 204264_at | 216033_s_at | 205329_s_at |
| 202291_s_at | 216640_s_at | 219165_at | 218110_at |
| 201121_s_at | 205317_s_at | 219489_s_at | 219732_at |
| 206813_at | 203576_at | 212221_x_at | 209110_s_at |
| 209546_s_at | 215812_s_at | 212503_s_at | 201586_s_at |
| 202117_at | 209142_s_at | 219370_at | 204985_s_at |
| 203501_at | 221003_s_at | 212111_at | 212953_x_at |
| 212518_at | 201675_at | 218454_at | 212316_at |
| 211944_at | 209971_x_at | 212158_at | 217970_s_at |
| 210968_s_at | 211758_x_at | 212586_at | 215519_x_at |
| 210628_x_at | 205246_at | 202643_s_at | 206254_at |
| 205044_at | 212032_s_at | 208306_x_at | 200098_s_at |
| 212119_at | 218567_x_at | 201730_s_at | 213490_s_at |
| 202450_s_at | 209180_at | 222240_s_at | 217959_s_at |
| 212179_at | 202886_s_at | 214660_at | 210434_x_at |
| 208335_s_at | 213687_s_at | 204790_at | 204340_at |
| 202464_s_at | 205084_at | 201311_s_at | 208799_at |
| 207118_s_at | 205687_at | 209967_s_at | 203316_s_at |
| 57715_at | 218493_at | 222024_s_at | 220742_s_at |
| 209263_x_at | 215091_s_at | 203749_s_at | 201780_s_at |
| 203071_at | 217846_at | 209596_at | 204343_at |
| 218667_at | 218563_at | 201721_s_at | 201931_at |
| 205805_s_at | 205145_s_at | 33322_i_at | 214167_s_at |
| 201605_x_at | 218548_x_at | 204794_at | 201016_at |
| 209343_at | 208852_s_at | 211796_s_at | 201479_at |
| 203518_at | 203317_at | 201696_at | 200055_at |
| 203597_s_at | 208864_s_at | 202172_at | 201826_s_at |
| 218892_at | 214117_s_at | 213249_at | 211033_s_at |
| 207542_s_at | 202923_s_at | 204260_at | 208800_at |
| 204310_s_at | 208436_s_at | 213170_at | 209739_s_at |
| 202765_s_at | 200831_s_at | 204344_s_at | 203272_s_at |
| 204491_at | 217127_at | 202208_s_at | 200087_s_at |
| 200611_s_at | 210312_s_at | 204294_at | 222356_at |
| 203156_at | 65133_i_at | 212120_at | 212527_at |
| 205201_at | 218503_at | 210632_s_at | 207181_s_at |
| 203339_at | 218321_x_at | 205478_at | 203246_s_at |
| 210915_x_at | 202300_at | 217795_s_at | 200942_s_at |
| 218723_s_at | 204391_x_at | 218902_at | 213245_at |
| 212878_s_at | 203133_at | 209312_x_at | 212219_at |
| 214085_x_at | 213720_s_at | 215306_at | 201066_at |
| 200905_x_at | 205244_s_at | 221898_at | 205355_at |
| 212197_x_at | 212340_at | 213519_s_at | 218732_at |
| 214894_x_at | 221511_x_at | 202908_at | 208959_s_at |
| 215543_s_at | 212165_at | 202305_s_at | 218448_at |
| 208634_s_at | 218357_s_at | 204803_s_at | 218816_at |
| 205857_at | 202710_at | 212353_at | 220925_at |
| 203889_at | 201630_s_at | 218152_at | 202138_x_at |
| 55081_at | 213843_x_at | 214771_x_at | 221620_s_at |
| 214608_s_at | 211708_s_at | 208760_at | 216958_s_at |
| 202931_x_at | 217284_x_at | 208502_s_at | 219041_s_at |
| 204730_at | 211177_s_at | 201743_at | 217824_at |
| 219304_s_at | 203581_at | 201120_s_at | 201011_at |
| 219024_at | 201463_s_at | 200985_s_at | 201830_s_at |
| 203028_s_at | 209545_s_at | 200816_s_at | 219819_s_at |
| 213316_at | 218857_s_at | 219985_at | 219913_s_at |
| 212549_at | 205980_s_at | 33323_r_at | 204466_s_at |
| 218196_at | 206724_at | 213348_at | 207721_x_at |
| 207966_s_at | 208801_at | 209645_s_at | 210186_s_at |
| 217226_s_at | 218010_x_at | 217997_at | 201772_at |
| 208633_s_at | 218016_s_at | 212561_at | 221588_x_at |
| 202878_s_at | 215280_s_at | 211998_at | 209776_s_at |
| 210202_s_at | 39817_s_at | 219534_x_at | 201653_at |
| 203233_at | 202119_s_at | 201648_at | 213379_at |
| 208615_s_at | 212751_at | 213309_at | 212246_at |
| 205782_at | 200873_s_at | 202821_s_at | 218112_at |
| 201752_s_at | 202737_s_at | 203264_s_at | 214240_at |
| 208835_s_at | 203827_at | 212071_s_at | 202666_s_at |
| 206710_s_at | 205750_at | 213182_x_at | 212563_at |
| 203639_s_at | 205294_at | 211990_at | 218969_at |
| 202422_s_at | 201268_at | 211974_x_at | 202299_s_at |
| 203068_at | 212053_at | 219221_at | 201819_at |
| 205898_at | 208264_s_at | 203964_at | 214542_x_at |
| 205577_at | 219125_s_at | 215706_x_at | 203605_at |
| 218376_s_at | 202502_at | 205348_s_at | 213116_at |
| 208146_s_at | 210859_x_at | 221816_s_at | 203918_at |
| 205882_x_at | 221786_at | 222158_s_at | 202195_s_at |
| 58916_at | 205613_at | 218823_s_at | 217870_s_at |
| 208848_at | 204333_s_at | 202156_s_at | 208702_x_at |
| 202180_s_at | 219342_at | 218804_at | 212406_s_at |
| 212604_at | 200961_at | 212923_s_at | 209998_at |
| 201859_at | 201597_at | 213901_x_at | 205709_s_at |
| 213075_at | 214140_at | 218656_s_at | 213836_s_at |
| 203017_s_at | 201619_at | 205961_s_at | 209864_at |
| 209374_s_at | 203544_s_at | 204993_at | 201947_s_at |
| 205933_at | 203177_x_at | 213620_s_at | 203360_s_at |
| 212510_at | 201523_x_at | 209379_s_at | 218046_s_at |
| 209086_x_at | 213132_s_at | 215146_s_at | 201733_at |
| 201869_s_at | 206307_s_at | 219228_at | 220945_x_at |
| 209786_at | 203024_s_at | 212253_x_at | 208764_s_at |
| 202432_at | 219283_at | 221676_s_at | 208843_s_at |
| 202341_s_at | 213166_x_at | 212681_at | 208639_x_at |
| 201958_s_at | 200910_at | 201137_s_at | 218174_s_at |
| 215333_x_at | 208638_at | 202242_at | 201549_x_at |
| 204655_at | 209921_at | 201037_at | 208654_s_at |
| 214721_x_at | 201410_at | 205011_at | 220721_at |
| 211991_s_at | 204426_at | 203695_s_at | 205486_at |
| 209298_s_at | 208826_x_at | 212350_at | 201216_at |
| 209787_s_at | 210627_s_at | 201559_s_at | 213059_at |
| 221884_at | 202983_at | 201995_at | 214779_s_at |
| 203685_at | 209175_at | 219936_s_at | 213017_at |
| 202008_s_at | 212767_at | 215193_x_at | 203997_at |
| 201968_s_at | 218375_at | 204759_at | 219787_s_at |
| 212430_at | 203880_at | 209846_s_at | 210136_at |
| 221870_at | 211971_s_at | 204640_s_at | 205807_s_at |
| 214121_x_at | 213152_s_at | 203178_at | 203415_at |
| 213547_at | 201622_at | 221666_s_at | 201096_s_at |
| 203813_s_at | 203379_at | 209568_s_at | 214472_at |
| 218675_at | 218681_s_at | 203604_at | 209872_s_at |
| 211986_at | 201359_at | 201566_x_at | 201972_at |
| 203619_s_at | 218647_s_at | 211026_s_at | 218001_at |
| 204028_s_at | 204123_at | 205624_at | 218944_at |
| 209691_s_at | 208951_at | 213135_at | 212311_at |
| 204140_at | 209036_s_at | 204735_at | 201486_at |
| 206453_s_at | 200967_at | 202132_at | 209593_s_at |
| 209612_s_at | 205938_at | 213015_at | 214895_s_at |
| 209197_at | 212109_at | 204049_s_at | 215125_s_at |
| 213306_at | 208886_at | AFFX- | |
| 202207_at | 221531_at | HSAC07/X00351_M_at | 205622_at |
| 213714_at | 200699_at | 219737_s_at | 221041_s_at |
| 208767_s_at | 220584_at | 37408_at | 220342_x_at |
| 202401_s_at | 215923_s_at | 213154_s_at | 213491_x_at |
| 201604_s_at | 201659_s_at | 213364_s_at | 217551_at |
| 218486_at | 208074_s_at | 206355_at | 206103_at |
| 212414_s_at | 213119_at | 201858_s_at | 205875_s_at |
| 221016_s_at | 217868_s_at | 203590_at | 212175_s_at |
| 201153_s_at | 202233_s_at | 205262_at | 203148_s_at |
| 220233_at | 210087_s_at | 202947_s_at | 203123_s_at |
| 202946_s_at | 219036_at | 212328_at | 209576_at |
| 209082_s_at | 218633_x_at | 204021_s_at | 218073_s_at |
| 215870_s_at | 202558_s_at | 200839_s_at | 214096_s_at |
| 203868_s_at | 208716_s_at | 203939_at | 201524_x_at |
| 222146_s_at | 202712_s_at | 216235_s_at | 208918_s_at |
| 203325_s_at | 214214_s_at | 214055_x_at | 203207_s_at |
| 205022_s_at | 201091_s_at | 212143_s_at | 218928_s_at |
| 221502_at | 213996_at | 208723_at | 221827_at |
| 202950_at | 221984_s_at | 204863_s_at | 218272_at |
| 202644_s_at | 214855_s_at | 205120_s_at | 53968_at |
| 202411_at | 203582_s_at | 218204_s_at | 220761_s_at |
| 205168_at | 214710_s_at | 213290_at | 209227_at |
| 213228_at | 200804_at | 212382_at | 201358_s_at |
| 201655_s_at | 209007_s_at | 221246_x_at | 213857_s_at |
| 207741_x_at | 219061_s_at | 202724_s_at | 209482_at |
| 222101_s_at | 218283_at | 221718_s_at | 204949_at |
| 204802_at | 216338_s_at | 201719_s_at | 219200_at |
| 214439_x_at | 200846_s_at | 212268_at | 205698_s_at |
| 218683_at | 210739_x_at | 209473_at | 201722_s_at |
| 209584_x_at | 210296_s_at | 201744_s_at | 208722_s_at |
| 205127_at | 202308_at | 203140_at | 204039_at |
| 210896_s_at | 202425_x_at | 213656_s_at | 203235_at |
| 209737_at | 212688_at | 203232_s_at | 217927_at |
| 211538_s_at | 203721_s_at | 200653_s_at | 204427_s_at |
| 219902_at | 219603_s_at | 204304_s_at | 218039_at |
| 209199_s_at | 201115_at | 203687_at | 201698_s_at |
| 205109_s_at | 203139_at | 212566_at | 208796_s_at |
| 200838_at | 206827_s_at | 201666_at | 202832_at |
| 91703_at | 222155_s_at | 212086_x_at | 218680_x_at |
| 212387_at | 214857_at | 218864_at | 201736_s_at |
| 203231_s_at | 221542_s_at | 205265_s_at | 205293_x_at |
| 203510_at | 208787_at | 204497_at | 217908_s_at |
| 222288_at | 220638_s_at | 213262_at | 202838_at |
| 201152_s_at | 205073_at | 209318_x_at | 218984_at |
| 216215_s_at | 205107_s_at | 201310_s_at | 216064_s_at |
| 205752_s_at | 65517_at | 218574_s_at | 206790_s_at |
| 221796_at | 209608_s_at | 215707_s_at | 210946_at |
| 212488_at | 211034_s_at | 201621_at | 201961_s_at |
| 205548_s_at | 213129_s_at | 212757_s_at | 215438_x_at |
| 212099_at | 217900_at | 204550_x_at | 210962_s_at |
| 205578_at | 218268_at | 207191_s_at | 218792_s_at |
| 201009_s_at | 205019_s_at | 203725_at | 201520_s_at |
| 201234_at | 219762_s_at | 213891_s_at | 202996_at |
| 206481_s_at | 213995_at | 210198_s_at | 218192_at |
| 218051_s_at | 202606_s_at | 33760_at | 218241_at |
| 218711_s_at | 202793_at | 204929_s_at | 204922_at |
| 205620_at | 200889_s_at | 212148_at | 203484_at |
| 202074_s_at | 202603_at | 220751_s_at | 202346_at |
| 212276_at | 216074_x_at | 201149_s_at | 209300_s_at |
| 210036_s_at | 219335_at | 205792_at | 218972_at |
| 204271_s_at | 202543_s_at | 222303_at | 201264_at |
| 213069_at | 204301_at | 209406_at | 200968_s_at |
| 209121_x_at | 213050_at | 213401_s_at | 211416_x_at |
| 209613_s_at | 220189_s_at | 202587_s_at | 212322_at |
| 204518_s_at | 221648_s_at | 203884_s_at | 209064_x_at |
| 207002_s_at | 201078_at | 210276_s_at | 204392_at |
| 213381_at | 218291_at | 209242_at | 212305_s_at |
| 211002_s_at | 211936_at | 221671_x_at | 217964_at |
| 201482_at | 202064_s_at | 209270_at | 204927_at |
| 209959_at | 203201_at | 212489_at | 202918_s_at |
| 201868_s_at | 205876_at | 210751_s_at | 209218_at |
| 45297_at | 200820_at | 202898_at | 210816_s_at |
| 204517_at | 211404_s_at | 201508_at | 209150_s_at |
| 210105_s_at | 218500_at | 201425_at | 209662_at |
| 202762_at | 201098_at | 204058_at | 218439_s_at |
| 216331_at | 221941_at | 203002_at | 203971_at |
| 213982_s_at | 212496_s_at | 219506_at | 212536_at |
| 209447_at | 202418_at | 202609_at | 213234_at |
| 212690_at | 208653_s_at | 218236_s_at | 201892_s_at |
| 201368_at | 205593_s_at | 203753_at | 218275_at |
| 212817_at | 220094_s_at | 205251_at | 218981_at |
| 214767_s_at | 204175_at | 201865_x_at | 214005_at |
| 213134_x_at | 220741_s_at | 204149_s_at | 203102_s_at |
| 202796_at | 203225_s_at | 203256_at | 208802_at |
| 212386_at | 219848_s_at | 205381_at | 210886_x_at |
| 216887_s_at | 203008_x_at | 215382_x_at | 218206_x_at |
| 203411_s_at | 217790_s_at | 205743_at | 218888_s_at |
| 201151_s_at | 202096_s_at | 201286_at | 213301_x_at |
| 209090_s_at | 201568_at | 221773_at | 210024_s_at |
| 209305_s_at | 201005_at | 208963_x_at | 200806_s_at |
| 212793_at | 205812_s_at | 206117_at | 214522_x_at |
| 210145_at | 209873_s_at | 216264_s_at | 200929_at |
| 216565_x_at | 209265_s_at | 201312_s_at | 213308_at |
| 221651_x_at | 213410_at | 203607_at | 201953_at |
| 204205_at | 221882_s_at | 215127_s_at | 200803_s_at |
| 203886_s_at | 219048_at | 221900_at | 202655_at |
| 37005_at | 218826_at | 201599_at | 218326_s_at |
| 205383_s_at | 201790_s_at | 201536_at | 205164_at |
| 201148_s_at | 218704_at | 207761_s_at | 206557_at |
| 201387_s_at | 218701_at | 1598_g_at | 205594_at |
| 206104_at | 219217_at | 212239_at | 208840_s_at |
| 204422_s_at | 216305_s_at | 221045_s_at | 202194_at |
| 210613_s_at | 204386_s_at | 209264_s_at | 214307_at |
| 201012_at | 203775_at | 212646_at | 214281_s_at |
| 212463_at | 202395_at | 212669_at | 204608_at |
| 219829_at | 200048_s_at | 218678_at | 208910_s_at |
| 205364_at | 203165_s_at | 218934_s_at | 200599_s_at |
| 221766_s_at | 218532_s_at | 202917_s_at | 204127_at |
| 203585_at | 220942_x_at | 215388_s_at | 202211_at |
| 202720_at | 210243_s_at | 202228_s_at | 210241_s_at |
| 203066_at | 210907_s_at | 202465_at | 202660_at |
| 208430_s_at | 219065_s_at | 204115_at | 212623_at |
| 204059_s_at | 221586_s_at | 214464_at | 212410_at |
| AFFX- | | 212805_at | 205077_s_at |
| HSAC07/X00351_5_at | 211747_s_at | 218421_at | 205538_at |
| 215464_s_at | 211754_s_at | 202157_s_at | 201219_at |
| 208965_s_at | 201339_s_at | 202388_at | 218883_s_at |
| 201185_at | 214875_x_at | 201008_s_at | 205160_at |
| 212195_at | 218213_s_at | 210471_s_at | 206299_at |
| 201272_at | 213365_at | 213993_at | 201401_s_at |
| 213158_at | 204967_at | 209135_at | 218328_at |
| 218502_s_at | 202406_s_at | 210072_at | 217871_s_at |
| 209287_s_at | 221688_s_at | 201867_s_at | 204332_s_at |
| 210517_s_at | 201943_s_at | 204037_at | 213600_at |
| 206359_at | 211497_x_at | 58780_s_at | 204331_s_at |
| 221276_s_at | 212741_at | 212240_s_at | 218003_s_at |
| 206022_at | 209250_at | 212358_at | 203431_s_at |
| 219647_at | 213399_x_at | 212845_at | 217986_s_at |
| 201289_at | 218989_x_at | 211962_s_at | 209759_s_at |
| 212535_at | 202296_s_at | 203810_at | 204160_s_at |
| 204114_at | 212307_s_at | 204455_at | 202960_s_at |
| 211984_at | 212116_at | 219427_at | 204142_at |
| 204755_x_at | 200636_s_at | 212203_x_at | 213518_at |
| 219505_at | 201284_s_at | 201329_s_at | 206429_at |
| 209604_s_at | 219920_s_at | 209200_at | 212685_s_at |
| 209883_at | 64486_at | 212354_at | 218676_s_at |
| 213004_at | 208872_s_at | 202766_s_at | 208612_at |
| 204621_s_at | 215227_x_at | 212077_at | 211574_s_at |
| 209505_at | 214358_at | 201389_at | 218608_at |
| 203636_at | 201135_at | 203688_at | 212064_x_at |
| 213110_s_at | 219076_s_at | 218435_at | 201955_at |
| 221583_s_at | 220625_s_at | 214724_at | 204233_s_at |
| 217023_x_at | 221920_s_at | 206932_at | 206351_s_at |
| 201602_s_at | 208689_s_at | 214077_x_at | 200052_s_at |
| 202086_at | 200863_s_at | 201315_x_at | 212749_s_at |
| 204688_at | 202857_at | 57588_at | 209326_at |
| 212151_at | 217645_at | 213274_s_at | 202279_at |
| 212554_at | 205937_at | 200808_s_at | 218145_at |
| 202759_s_at | 212279_at | 201109_s_at | 200895_s_at |
| 202794_at | 221637_s_at | 207547_s_at | 201004_at |
| 211564_s_at | 209796_s_at | 202728_s_at | 218049_s_at |
| 203570_at | 201962_s_at | 213016_at | 201941_at |
| 201850_at | 202785_at | 204072_s_at | 211899_s_at |
| 203088_at | 201976_s_at | 217890_s_at | 218027_at |
| 209047_at | 218962_s_at | 212526_at | 221739_at |
| 212274_at | 217755_at | 206211_at | 217483_at |
| 203254_s_at | 203524_s_at | 200904_at | 220753_s_at |
| 205303_at | 218961_s_at | 209293_x_at | 208950_s_at |
| 206874_s_at | 50400_at | 212501_at | 207655_s_at |
| 212587_s_at | 219362_at | 205304_s_at | 200807_s_at |
| 212190_at | 213988_s_at | 216733_s_at | 212922_s_at |
| 204777_s_at | 217962_at | 209897_s_at | 221823_at |
| 212242_at | 218194_at | 203620_s_at | 213713_s_at |
| 206701_x_at | 200652_at | 203637_s_at | 212314_at |
| 213974_at | 218557_at | 209470_s_at | 208309_s_at |
| 202686_s_at | 201791_s_at | 204990_s_at | 219133_at |
| 218298_s_at | 210018_x_at | 219179_at | 213501_at |
| 217996_at | 217800_s_at | 213438_at | 209149_s_at |
| 212344_at | 204905_s_at | 218499_at | 204238_s_at |
| 210084_x_at | 220642_x_at | 213275_x_at | 213280_at |
| 211323_s_at | 214315_x_at | 201060_x_at | 215471_s_at |
| 221755_at | 204168_at | 201565_s_at | 203116_s_at |
| 204749_at | 217956_s_at | 203295_s_at | 209357_at |
| 202071_at | 213441_x_at | 201069_at | 218592_s_at |
| 205051_s_at | 222262_s_at | 203921_at | 215696_s_at |
| 204418_x_at | 220892_s_at | 208816_x_at | 204404_at |
| 204099_at | 201890_at | 202554_s_at | 218261_at |
| 209663_s_at | 218996_at | 211981_at | 208583_x_at |
| 218854_at | 202836_s_at | 221814_at | 212186_at |
| 208944_at | 209224_s_at | 201601_x_at | 203641_s_at |
| 211671_s_at | 218923_at | 214022_s_at | 210541_s_at |
| 201136_at | 91816_f_at | 209285_s_at | 206352_s_at |
| 214071_at | 200825_s_at | 202760_s_at | 202721_s_at |
| 205683_x_at | 200093_s_at | 209101_at | 218546_at |
| 210095_s_at | 219166_at | 212886_at | 222216_s_at |
| 205433_at | 218789_s_at | 219440_at | 218652_s_at |
| 212624_s_at | 217825_s_at | 203640_at | 219301_s_at |
| 204687_at | 205757_at | 209656_s_at | 209164_s_at |
| 213411_at | 203517_at | 206377_at | 209694_at |
| 218223_s_at | 207809_s_at | 203632_s_at | 221345_at |
| 212677_s_at | 212570_at | 209154_at | 202778_s_at |
| 208636_at | 203224_at | 201560_at | 217803_at |
| 204352_at | 202961_s_at | 201426_s_at | 201912_s_at |
| 201328_at | 219115_s_at | 213675_at | 211075_s_at |
| 213010_at | 200044_at | 211577_s_at | 202540_s_at |
| 207134_x_at | 220080_at | 217764_s_at | 217851_s_at |
| 218330_s_at | 222118_at | 202664_at | 214274_s_at |
| 211160_x_at | 203629_s_at | 210764_s_at | 208398_s_at |
| 213005_s_at | 201940_at | 202551_s_at | 214097_at |
| 65718_at | 207414_s_at | 213001_at | 219038_at |
| 204223_at | 205768_s_at | 218901_at | 218605_at |
| 212419_at | 221590_s_at | 212104_s_at | 209502_s_at |
| 202732_at | 203931_s_at | 208228_s_at | 219276_x_at |
| 219922_s_at | 216251_s_at | 209583_s_at | 214157_at |
| 201603_at | 218387_s_at | 209469_at | 222125_s_at |
| 201243_s_at | 220980_s_at | 217762_s_at | 202889_x_at |
| 211535_s_at | 203557_s_at | 202729_s_at | 218865_at |
| 205802_at | 208841_s_at | 218285_s_at | 217758_s_at |
| 216474_x_at | 219551_at | 212764_at | 210371_s_at |
| 201170_s_at | 209147_s_at | 221760_at | 203228_at |
| 212675_s_at | 218458_at | 219064_at | 201543_s_at |
| 214696_at | 212202_s_at | 216321_s_at | 211498_s_at |
| 204430_s_at | 207949_s_at | 204754_at | 211778_s_at |
| 209205_s_at | 201579_at | 221584_s_at | 203594_at |
| 222108_at | 200894_s_at | 209466_x_at | 212474_at |
| 37996_s_at | 202939_at | 204424_s_at | 214437_s_at |
| 208370_s_at | 206656_s_at | 204748_at | 203663_s_at |
| 214266_s_at | 200852_x_at | 212647_at | 212652_s_at |
| 221127_s_at | 200947_s_at | 202719_s_at | 218434_s_at |
| 209016_s_at | 209665_at | 211985_s_at | 211715_s_at |
| 201841_s_at | 202941_at | 212423_at | 203115_at |
| 208949_s_at | 209605_at | 209436_at | 201647_s_at |
| 201369_s_at | 211733_x_at | 204268_at | 202718_at |
| 209655_s_at | 212347_x_at | 208690_s_at | 212204_at |
| 203603_s_at | 213244_at | 217763_s_at | 211417_x_at |
| 205803_s_at | 221428_s_at | 204971_at | 217168_s_at |
| 206433_s_at | 209108_at | 219410_at | 212989_at |
| 212914_at | 201825_s_at | 212993_at | 209228_x_at |
| 203748_x_at | 203545_at | 206580_s_at | 221245_s_at |
| 218824_at | 203616_at | 204472_at | 203124_s_at |
| 205608_s_at | 201116_s_at | 201430_s_at | 210996_s_at |
| 201313_at | 220226_at | 211562_s_at | 201760_s_at |
| 202075_s_at | 200654_at | 204163_at | 209919_x_at |
| 204396_s_at | 205925_s_at | 202133_at | 213812_s_at |
| 209465_x_at | 218720_x_at | 201215_at | 205155_s_at |
| 213924_at | 217894_at | 218094_s_at | 205420_at |
| 207935_s_at | 217942_at | 204753_s_at | 207131_x_at |
| 218162_at | 212160_at | 204442_x_at | 202843_at |
| 213194_at | 218654_s_at | 203680_at | 210547_x_at |
| 205952_at | 211297_s_at | 213400_s_at | 211576_s_at |
| 206391_at | 202599_s_at | 202403_s_at | 217919_s_at |
| 218518_at | 217761_at | 217437_s_at | 201761_at |
| 211965_at | 218966_at | 209868_s_at | 220547_s_at |
| 214104_at | 202178_at | 210096_at | 221923_s_at |
| 205200_at | 214109_at | 213524_s_at | 212694_s_at |
| 209621_s_at | 218140_x_at | 202949_s_at | 201661_s_at |
| 208962_s_at | 203630_s_at | 205934_at | 208523_x_at |
| 209821_at | 200698_at | 212509_s_at | 209905_at |
| 212713_at | 201127_s_at | 201030_x_at | 218388_at |
| 212736_at | 212916_at | 200696_s_at | 203009_at |
| 202822_at | 205074_at | 202177_at | 209109_s_at |
| 212848_s_at | 207606_s_at | 209542_x_at | 203765_at |
| 207266_x_at | 214919_s_at | 208029_s_at | 209917_s_at |
| 201300_s_at | 202183_s_at | 212288_at | 209916_at |
| 204855_at | 217043_s_at | 204940_at | 208783_s_at |
| 212135_s_at | 211048_s_at | 210427_x_at | 207260_at |
| 212667_at | 207981_s_at | 201893_x_at | 207980_s_at |
| 205573_s_at | 218582_at | 205083_at | 212680_x_at |
| 209337_at | 214243_s_at | 206392_s_at | 220030_at |
| 200911_s_at | 205003_at | 204793_at | 219649_at |
| 206631_at | 213900_at | 213800_at | 204170_s_at |
| 213572_s_at | 203215_s_at | 207016_s_at | 217826_s_at |
| 201792_at | 218423_x_at | 210986_s_at | 209302_at |
| 212551_at | 217749_at | 208637_x_at | 203387_s_at |
| 219654_at | 214308_s_at | 211864_s_at | 209836_x_at |
| 200878_at | 212816_s_at | 200795_at | 202016_at |
| 211980_at | 215794_x_at | 202393_s_at | 221610_s_at |
| 205229_s_at | 221782_at | 211737_x_at | 202539_s_at |
| 219935_at | 218931_at | 204938_s_at | 203966_s_at |
| 823_at | 201197_at | 219090_at | 211935_at |
| 202073_at | 201691_s_at | 201617_x_at | 202109_at |
| 204602_at | 201900_s_at | 214039_s_at | 209600_s_at |
| 213258_at | 203011_at | 220532_s_at | 201013_s_at |
| 220765_s_at | 220816_at | 203370_s_at | 220187_at |
| 209550_at | 222140_s_at | 209863_s_at | 213143_at |
| 214761_at | 200946_x_at | 215813_s_at | 218218_at |
| 212361_s_at | 204026_s_at | 201798_s_at | 204567_s_at |
| 212091_s_at | 218465_at | 200824_at | 205 309_at |
| 201462_at | 208284_x_at | 211966_at | 201 735_s_at |
| 210987_x_at | 203138_at | 204359_at | 206170_at |
| 211813_x_at | 221754_s_at | 211964_at | 201704_at |
| 2051 28_x_at | 200903_s_at | 200600_at | 220606_s_at |
| 207836_s_at | 204143_s_at | 213 338_at | 221788_at |
| 203705_s_at | 211494_s_at | 201616_s_at | 205833_s_at |
| 204030_s_at | 218924_s_at | 200982_s_at | 202061_s_at |
| 214265_at | 207431_s_at | 201061_s_at | 204957_at |
| 213503_x_at | 202871_at | 206434_at | 209113_s_at |
| 209356_x_at | 206385_s_at | 207826_s_at | 205042_at |
| 201590_x_at | 203130_s_at | 204345_at | 203 593_at |
| 203638_s_at | 221027_s_at | 202920_at | 216483_s_at |
| 213156_at | 201734_at | 213293_s_at | 212692_s_at |
| 204412_s_at | 219395_at | 206332_s_at | 214446_at |
| 202504_at | 205078_at | 203 710_at | 204121_at |
| 212887_at | 213423_x_at | 218974_at | 206069_s_at |
| 216598_s_at | 219152_at | 200974_at | 212573_at |
| 211343_s_at | 213943_at | 205384_at | 212899_at |
| 203892_at | 219121_s_at | 203571_s_at | 202363_at |
| 219747_at | 207362_at | 210078_s_at | 207824_s_at |
| 209118_s_at | 209772_s_at | 202350_s_at | 219933_at |
| 218694_at | 207549_x_at | 206070_s_at | 218556_at |
| 211340_s_at | 201660_at | 208789_at | 202929_s_at |
| 209087_x_at | 2053 16_at | 218963_sat | 219555_s_at |
| 204963_at | 212282_at | 207961_x_at | 221927_s_at |
| 209191_at | 218531_at | 207957_s_at | 213 148_at |
| 209129_at | 200681_at | 200930_s_at | 202503_s_at |
| 204964_s_at | 205566_at | 204041_at | 209625_at |
| 217767_at | 203164_at | 221935_s_at | 210108_at |
| 213564_x_at | 202023_at | 202994_s_at | 209504_s_at |
| 221872_at | 2072 75_s_at | 209488_s_at | 222315_at |
| 203562_at | 201130_s_at | 218224_at | 218979_at |
| 209685_s_at | 2 17823_s_at | 204731_at | 201577_at |
| 219250_s_at | 221781_s_at | 203498_at | 215407_s_at |
| 204036_at | 37117_at | 203881_s_at | 205133_s_at |
| 211126_s_at | 205942_s_at | 201147_s_at | 209367_at |
| 201438_at | 2 15380_s_at | 213994_s_at | 200970_s_at |
| 214212_x_at | 2 19518_s_at | 206938_at | 202605_at |
| 213568_at | 200971_s_at | 205609_at | 63825_at |
| 201631_s_at | 221874_at | 201645_at | 205505_at |
| 202440_s_at | 212978_at | 209496_at | 218025_s_at |
| 212977_at | 210720_s_at | 212067_s_at | 206110_at |
| 221541_at | 218188_s_at | 204364_s_at | 204942_s_at |
| 200923_at | 201724_s_at | 212236_x_at | 217111_at |
| 220595_at | 208737_at | 212813_at | 203219_s_at |
| 204284_at | 218909_at | 218380_at | 204019_s_at |
| 208747_s_at | 209531_at | 212230_at | 212295_s_at |
| 203131_at | 201417_at | 218418_s_at | 209855_s_at |
| 201242_s_at | 202893_at | 205132_at | 221024_s_at |
| 204463_s_at | 218086_at | 200931_s_at | 221865_at |
| 204464_s_at | 51158_at | 209427_at | 203386_at |
| 201843_s_at | 219411_at | 204288_s_at | 210719_s_at |
| 202748_at | 218258_at | 218730_s_at | 221880_s_at |
| 202018_s_at | 201583_s_at | 218980_at | 220432_s_at |
| 208966_x_at | 209825_s_at | 213371_at | 202546_at |
| 209209_s_at | 222121_at | 203706_s_at | 211423_s_at |
| 200897_s_at | 204388_s_at | 205856_at | 217736_s_at |
| 209487_at | 219850_s_at | 221748_s_at | 207098_s_at |
| 210869_s_at | 204389_at | 200907_s_at | 200606_at |
| 211896_s_at | 215108_x_at | 222162_s_at | 219388_at |
| 219295_s_at | 201196_s_at | 209286_at | 213085_s_at |
| 209335_at | 209478_at | 204955_at | 200078_s_at |
| 211663_x_at | 214733_s_at | 212843_at | 206860_s_at |
| 202566_s_at | 205769_at | 205157_s_at | 202668_at |
| 204570_at | 209030_s_at | 204069_at | 218248_at |
| 209074_s_at | 201014_s_at | 200953_s_at | 219584_at |
| 201348_at | 202005_at | 203851_at | 211559_s_at |
| 201957_at | 206068_s_at | 205725_at | 206303_s_at |
| 202202_s_at | 203029_s_at | 212226_s_at | 205248_at |
| 213428_s_at | 203430_at | 208131_s_at | 217776_at |
| 201497_x_at | 219015_s_at | 200621_at | 201963_at |
| 213992_at | 200700_s_at | 211748_x_at | 202769_at |
| 218611_at | 212181_s_at | 207977_s_at | 213325_at |
| 212254_s_at | 205102_at | 207876_s_at | 209585_s_at |
| 209948_at | 204319_s_at | 206116_s_at | 208580_x_at |
| 217757_at | 200670_at | 204273_at | 202790_at |
| 204457_s_at | 266_s_at | 201787_at | 204141_at |
| 221505_at | 210787_s_at | 209651_at | 218696_at |
| 201540_at | 206770_s_at | 204931_at | 209514_s_at |
| 200986_at | 214106_s_at | 202283_at | 210480_s_at |
| 200906_s_at | 203042_at | 209687_at | 212744_at |
| 203729_at | 210715_s_at | 201842_s_at | 209934_s_at |
| 218718_at | 212448_at | 201431_s_at | 215432_at |
| 214091_s_at | 212115_at | 209156_s_at | 202428_x_at |
| 202196_s_at | 87100_at | 202269_x_at | 217014_s_at |
| 204400_at | 200656_s_at | 202007_at | 209693_at |
| 201105_at | 213892_s_at | 219167_at | 211596_s_at |
| 209288_s_at | 208658_at | 201150_s_at | 222258_s_at |
| 214505_s_at | 203030_s_at | 202565_s_at | 204394_at |
| 200762_at | 220014_at | 209616_s_at | 208788_at |
| 212136_at | 217912_at | 214247_s_at | 213288_at |
| 203423_at | 210293_s_at | 209283_at | 209031_at |
| 201641_at | 211724_x_at | 212187_x_at | 221589_s_at |
| 213093_at | 202148_s_at | 217728_at | 213712_at |
| 202995_s_at | 221019_s_at | 201539_s_at | 201951_at |
| 204939_s_at | 212183_at | 210298_x_at | 203180_at |
| 204894_s_at | 201193_at | 205547_s_at | 208190_s_at |
| 215016_x_at | 201582_at | 207030_s_at | 203642_s_at |
| 210139_s_at | 208527_x_at | 209167_at | 218211_s_at |
| 219685_at | 202770_s_at | 209291_at | 202826_at |
| 201495_x_at | 210951_x_at | 213068_at | 208180_s_at |
| 203065_s_at | 212745_s_at | 209351_at | 219017_at |
| 205549_at | 207843_x_at | 209170_s_at | 219405_at |
| 203324_s_at | 217775_s_at | 202222_s_at | 205645_at |
| 219478_at | 40093_at | 202992_at | 203717_at |
| 209210_s_at | 212252_at | 213746_s_at | 201079_at |
| 203323_at | 204776_at | 208791_at | 209389_x_at |
| 212768_s_at | 210738_s_at | 208792_s_at | 210041_s_at |
| 204135_at | 222067_x_at | 205564_at | 202688_at |
| 213071_at | 201848_s_at | 204734_at | 210652_s_at |
| 202274_at | 205221_at | 201058_s_at | 203946_s_at |
| 209540_at | 209366_x_at | 205382_s_at | 202088_at |
| 209355_s_at | 219266_at | 205242_at | 202457_s_at |
| 33767_at | 210337_s_at | 201496_x_at | 200832_s_at |
| 201615_x_at | 201131_s_at | | 202722_s_at |
| 209541_at | 202786_at | | 209706_at |
| 212724_at | 208546_x_at | | 204583_x_at |
| 213139_at | 202740_at | | 220933_s_at |
| 212233_at | 220926_s_at | | 214404_x_at |
| 203903_s_at | 211070_x_at | | 213246_at |
| 207480_s_at | 213920_at | | 222209_s_at |
| 208790_s_at | 209094_at | | 200969_at |
| 210299_s_at | 220380_at | | 213285_at |
| 221747_at | 215779_s_at | | 202429_s_at |
| 205935_at | 202708_s_at | | 210387_at |
| 201820_at | 213106_at | | 203911_at |
| 209292_at | 200790_at | | 217875_s_at |
| 212992_at | 209911_x_at | | 221802_s_at |
| 202409_at | 208490_x_at | | 201128_s_at |
| 203766_s_at | 204751_x_at | | 219118_at |
| 203186_s_at | 212310_at | | 219667_s_at |
| 212730_at | 203041_s_at | | 210130_s_at |
| 212097_at | 216623_x_at | | 203739_at |
| 217897_at | 214329_x_at | | 204231_s_at |
| 203951_at | 212281_s_at | | 215726_s_at |
| 200859_x_at | 210317_s_at | | 205052_at |
| 222043_at | 217850_at | | 214765_s_at |
| 221667_s_at | 218922_s_at | | 201849_at |
| 211276_at | 213555_at | | 209460_at |
| 201667_at | 201413_at | | 222277_at |
| 214752_x_at | 217752_s_at | | 213587_s_at |
| 212865_s_at | 210222_s_at | | 210377_at |
| 218087_s_at | 204582_s_at | | 213622_at |
| 203296_s_at | 221561_at | | 222075_s_at |
| 208937_s_at | 202286_s_at | | 202525_at |
| 214027_x_at | 74694_s_at | | 204485_s_at |
| 202555_s_at | 209806_at | | 212543_at |
| 207390_s_at | 209163_at | | 220116_at |
| 209763_at | 212255_s_at | | 214774_x_at |
| 204083_s_at | 205924_at | | 203304_at |
| | 208650_s_at | | 218035_s_at |
| | 203644_s_at | | 201596_x_at |
| | 217901_at | | 205597_at |
| | 214463_x_at | | 209844_at |
| | 219127_at | | 217973_at |
| | 201562_s_at | | 209459_s_at |
| | 219117_s_at | | 202427_s_at |
| | 218254_s_at | | 214290_s_at |
| | 221582_at | | 214469_at |
| | 209696_at | | 219312_s_at |
| | 216905_s_at | | 209623_at |
| | 200935_at | | 219736_at |
| | 203485_at | | 211137_s_at |
| | 202687_s_at | | 46323_at |
| | 212640_at | | 219856_at |
| | 202089_s_at | | 218186_at |
| | 218189_s_at | | 206302_s_at |
| | 214651_s_at | | 212686_at |
| | 201952_at | | 203007_x_at |
| | 215017_s_at | | 202454_s_at |
| | 208837_at | | 206558_at |
| | 203857_s_at | | 202043_s_at |
| | 212812_at | | 214087_s_at |
| | 209935_at | | 205830_at |
| | 201662_s_at | | 209173_at |
| | 204973_at | | 205780_at |
| | 200644_at | | 218280_x_at |
| | 204305_at | | 204875_s_at |
| | 220161_s_at | | 209369_at |
| | 201923_at | | 202890_at |
| | 221732_at | | 205776_at |
| | 208579_x_at | | 212789_at |
| | 219806_s_at | | 221669_s_at |
| | 202489_s_at | | 218638_s_at |
| | 201563_at | | 217979_at |
| | 217080_s_at | | 36830_at |
| | 214455_at | | 218835_at |
| | 210328_at | | 203954_x_at |
| | 211478_s_at | | 210339_s_at |
| | 209340_at | | 203397_s_at |
| | 210788_s_at | | 220192_x_at |
| | 203716_s_at | | 209114_at |
| | 206214_at | | 209398_at |
| | 219476_at | | 212449_s_at |
| | 204667_at | | 211689_s_at |
| | 215071_s_at | | 203216_s_at |
| | 209854_s_at | | 206858_s_at |
| | 203917_at | | 212445_s_at |
| | 205862_at | | 201690_s_at |
| | 200862_at | | 212412_at |
| | 203474_at | | 203243_s_at |
| | 209624_s_at | | 211303_x_at |
| | 212218_s_at | | 204623_at |
| | 201688_s_at | | 215363_x_at |
| | 205542_at | | 205347_s_at |
| | 201839_s_at | | 219360_s_at |
| | 202345_s_at | | 203196_at |
| | 213506_at | | 203953_s_at |
| | 218313_s_at | | 205860_x_at |
| | 214598_at | | 216920_s_at |
| | 221424_s_at | | 215806_x_at |
| | 217487_x_at | | 221577_x_at |
| | 216804_s_at | | 211144_x_at |
| | 201689_s_at | | 209813_x_at |
| | 204934_s_at | | 209425_at |
| | 217771_at | | 209426_s_at |
| | 203908_at | | 209424_s_at |
| | 203242_s_at | | |

TABLE 7A
Tissue (tumor or stroma) specific genes used for prediction. Regular font:
up-regulated genes. Italics: down-regulated genes. Tumor Specific Gene List 1 - genes
used for tumor percentage prediction based on models developed by dataset 1. Tumor
Specific Gene List 2 - genes used for tumor percentage prediction based on models
developed by dataset 2. Stroma Specific Gene List 1 - genes used for stroma percentage
prediction based on models developed by dataset 1. Stroma Specific Gene List 2 - genes
used for stroma percentage prediction based on models developed by dataset 2.

| Tumor Specific | | Tumor Specific | Stroma Specific | Stroma Specific |
|---|---|---|---|---|
| Gene List 1 | | Gene List 2 | Gene List 1 | Gene List 2 |
| 211194_s_at | 201739_at | 214460_at | 202088_at | 209854_s_at |
| 202310_s_at | 209854_s_at | 201394_s_at | 200931_s_at | 200795_at |
| 216062_at | 33322_i_at | 202525_at | 209854_s_at | 207169_x_at |
| 211872_s_at | 209706_at | 201577_at | 205780_at | 212647_at |
| 215240_at | 205780_at | 205645_at | 217487_x_at | 201131_s_at |
| 204748_at | 205780_at | 203425_s_at | 221788_at | 214800_x_at |
| 204742_s_at | 201577_at | 202404_s_at | 202089_s_at | 202404_s_at |
| 204926_at | 209706_at | 200795_at | 211194_s_at | 219960_s_at |
| 205042_at | 200931_s_at | 214800_x_at | | 201615_x_at |
| 222043_at | 202088_at | 207169_x_at | | 205541_s_at |
| 212984_at | 202436_s_at | 209854_s_at | | 203084_at |
| 215775_at | 209283_at | | | 207956_x_at |
| 204742_s_at | 202088_at | | | 201995_at |
| 203698_s_at | 202088_at | | | 205645_at |
| 209771_x_at | 215350_at | | | 201577_at |
| 202089_s_at | | | | 201394_s_at |
| 209771_x_at | | | | 202525_at |
| 201839_s_at | | | | 214460_at |
| 205834_s_at | | | | |
| 209935_at | | | | |
| 211834_s_at | | | | |
| 221788_at | | | | |
| 210930_s_at | | | | |
| 212230_at | | | | |
| 202089_s_at | | | | |
| 201409_s_at | | | | |
| 201555_at | | | | |
| 33322_i_at | | | | |
| 217487_x_at | | | | |
| 201744_s_at | | | | |
| 201215_at | | | | |
| 211748_x_at | | | | |
| 221788_at | | | | |
| 215564_at | | | | |
| 201555_at | | | | |
| 33322_i_at | | | | |
| 211964_at | | | | |

TABLE 7B
Tissue (tumor or stroma) specific genes identified from dataset 2
used for prediction.

| Tumor specific, | Tumor specific, | Stroma specific, | Stroma specific, |
|---|---|---|---|
| up-regulated | down-regulated | up-regulated | down regulated |
| SIM2 | EXT1 | TBXA2R | STRA13 |
| AMACR | ANXA2 | XLKD1 | ZABC1 |
| MKI67 | TIMP2 | DCC | SIAT1 |
| CRISP3 | KIAA0172 | SLIT3 | ARFIP2 |
| HOXC6 | VCL | FGF18 | SLC39A6 |
| RET_var1 | MET | STAC | TUSC3 |
| DNAH5 | ILK | GNAZ | STEAP2 |
| MELK | TGFB2 | NTRK3 | CAMKK2 |
| HPN_var1 | STOM | SYNE1 | BNIP3 |
| PCGEM1 | MLCK | DAT1 | BDH |
| GI_2094528 | TGFBR3 | MAL | REPS2 |
| TMSNB | MEIS2 | NGFB | GDF15 |
| MYBL2 | KIP2 | DF | TMEPAI |
| UBE2C | PDLIM7 | SIAT7D | ATP2C1 |
| FOLH1 | PPAP2B | NTN1 | GI_22761402 |
| DKFZp434C0931 | IGF2 | CES1 | GI_4884218 |
| F5 | UB1 | ZAKI-4 | memD |
| HPN_var2 | CRYAB | FGF2 | tom1-like |
| RAB3B | CNN1 | G6PD | TNFSF10 |
| HNF-3-alpha | FZD7 | EDNRB | PRSS8 |
| EZH2 | KAI1 | IFI27 | MCCC2 |
| ECT2 | NBL1 | GSTP1 | TFAP2C |
| CDC6 | MMP2 | GSTM4 | ACPP |
| NY-REN-41 | SERPINF1 | GAS1 | DHCR24 |
| GPR43 | UNC5C | ITGA5 | MLP |
| NETO2 | CAV2 | RRAS | ERBB3 |
| D-PCa-2_mRNA | HNMP-1 | BC008967 | LIPH |
| BIK | GJA1 | MMP2 | PYCR1 |
| GALNT3 | TGFB3 | ITGB3 | NSP |
| PTTG1 | ITPR1 | AKAP2 | LOC129642 |
| FBP1 | GSTM3 | LAMA4 | CLUL1 |
| rap1GAP | CLU | BCL2_beta | TSPAN-1 |
| GI_3360414 | TU3A | SOLH | NKX3-1 |
| KIAA0869 | CAV1 | UNC5C | hAG-2/R |
| MLP | GSTM4 | CAV1 | hRVP1 |
| TACSTD1 | ZAKI-4 | KIAK0002 | CDH1 |
| GI_10437016 | TGFB2_cds | CLU | MOAT-B |
| MCCC2 | LTBP4 | PLS3 | SYT7 |
| STEAP | ITGB3 | ITPR1 | KLK4 |
| LOC129642 | BC008967 | HNMP-1 | STEAP |
| GI_4884218 | KIAK0002 | COL4A2 | NY-REN-41 |
| ERBB3 | GSTM5 | FZD7 | GI_3360414 |
| KIAA0389 | EDNRB | GSTM5 | GI_10437016 |
| PYCR1 | KIAA0003 | LOC119587 | FBP1 |
| memD | PTGS2 | LTBP4 | NETO2 |
| GI_22761402 | RRAS | HGF | BMPR1B |
| LIM | GAS1 | CAV2 | GPR43 |
| GALNT1 | G6PD | TRAF5 | TACSTD1 |
| BMPR1B | ALDH1A2 | COL5A2 | MYBL2 |
| SLC43A1 | FGF2 | GJA1 | GALNT3 |
| MCM2 | LSAMP | TGFB2_cds | KIAA0869 |
| COBLL1 | BCL2_beta | KIAA0003 | ESM1 |
| REPS2 | MAL | KIP2 | UBE2C |
| NKX3-1 | ITGA5 | UB1 | F5 |
| NME1 | FGFR2 | GSTM3 | D-PCa-2_var2 |
| DKFZP564B167 | FGF18 | CRYAB | GI_2094528 |
| HSD17B4 | SLIT3 | ANTXR1 | MELK |
| TMEPAI | TRIM29 | CNN1 | HOXC6 |
| CAMKK2 | SIAT7D | TU3A | SPDEF |
| GDF15 | GSTP1 | IGF2 | RET_var1 |
| P1 | GNAZ | SERPINF1 | rap1GAP |
| PAICS | XLKD1 | PDLIM7 | HPN_var2 |
| | NTRK3 | PPAP2B | BIK |
| | DF | TGFBR3 | MKI67 |
| | CES1 | GI_2056367 | HNF-3-alpha |
| | SYNE1 | ANGPTL2 | D-PCa-2_var1 |
| | NTN1 | ILK | D-PCa-2_mRNA |
| | SRD5A2 | ITSN | TRPM8 |
| | DCC | COL1A1 | DNAH5 |
| | STAC | STOM | CRISP3 |
| | TBXA2R | VCL | RAB3B |
| | CCK | KAI1 | AMACR |
| | | CAPL | HPN_var1 |
| | | MLCK | TMSNB |
| | | KIAA0172 | FOLH1 |
| | | SPARCL1 | PCGEM1 |
| | | MMP14 | DD3 |
| | | TIMP2 | SIM2 |
| | | CALM1 | |
| | | MEIS2 | |
| | | EXT1 | |

TABLE 8A
Tissue (tumor or stroma) specific relapse related genes.

| Tumor Specific Relapse Related Genes | | | Stroma Specific Relapse Related Genes | | |
|---|---|---|---|---|---|
| U95 Probe | U133 Probe | | U95 Probe | U133 Probe | |
| Set ID | Set ID | Gene Symbol | Set ID | Set ID | Gene Symbol |
| 1019_g_at | 206213_at | WNT10B | 1019_g_at | 206213_at | WNT10B |
| 1042_at | 206392_s_at | RARRES1 | 1050_at | 206426_at | MLA |
| 1052_s_at | 203973_s_at | CEBPD | 1051_g_at | 206426_at | MLA |
| 1078_at | 206346_at | PRLR | 1052_s_at | 203973_s_at | CEBPD |
| 1079_g_at | 206346_at | PRLR | 1134_at | 203839_s_at | TNK2 |
| 1087_at | 209962_at | EPOR | 1157_s_at | 204191_at | IFR1 |
| 1087_at | 209963_s_at | EPOR | 1176_at | 216261_at | ITGB3 |
| 1158_s_at | 200623_s_at | CALM3 | 117_at | 213418_at | HSPA6 |
| 1162_g_at | 203307_at | GNL1 | 1206_at | 204247_s_at | CDK5 |
| 1206_at | 204247_s_at | CDK5 | 1229_at | 205076_s_at | MTMR11 |
| 1229_at | 205076_s_at | MTMR11 | 1278_at | 202686_s_at | AXL |
| 54581_at | 213900_at | C9orf61 | 54581_at | 213900_at | C9orf61 |
| 54673_s_at | 218221_at | ARNT | 1284_at | 211084_x_at | PRKD3 |
| 54690_at | 210674_s_at | | 1318_at | 217301_x_at | RBBP4 |
| 1318_at | 217301_x_at | RBBP4 | 1337_s_at | 211605_s_at | RARA |
| 1343_s_at | 209720_s_at | SERPINB3 | 1343_s_at | 209720_s_at | SERPINB3 |
| 1368_at | 202948_at | IL1R1 | 1368_at | 202948_at | IL1R1 |
| 1385_at | 201506_at | TGFBI | 1385_at | 201506_at | TGFBI |
| 1397_at | 203652_at | MAP3K11 | 1408_at | 206783_at | FGF4 |
| 1398_g_at | 203652_at | MAP3K11 | 1460_g_at | 205171_at | PTPN4 |
| 139_at | 206490_at | DLGAP1 | 1536_at | 203967_at | CDC6 |
| 1456_s_at | 206332_s_at | IFI16 | 1543_at | 205699_at | — |
| 1456_s_at | 208966_x_at | IFI16 | 1560_g_at | 205962_at | PAK2 |
| 1499_at | 200090_at | FNTA | 1565_s_at | 215075_s_at | GRB2 |
| 1499_at | 200090_at | FNTA | 1598_g_at | 202177_at | GAS6 |
| 1504_s_at | 207501_s_at | FGF12 | 1610_s_at | 202533_s_at | DHFR /// |
| | | | | | LOC643509 /// |
| | | | | | LOC653874 |
| 1507_s_at | 204464_s_at | EDNRA | 1707_g_at | 201895_at | ARAF |
| 1536_at | 203967_at | CDC6 | 1747_at | 214992_s_at | DSE2 |
| 1543_at | 205699_at | — | 1747_at | 209831_x_at | DSE2 |
| 1565_s_at | 215075_s_at | GRB2 | 1749_at | 208369_s_at | GCDH |
| 1575_at | 209993_at | ABCB1 | 1749_at | 203500_at | GCDH |
| 1576_g_at | 209993_at | ABCB1 | 1754_at | 201763_s_at | DAXX |
| 1598_g_at | 202177_at | GAS6 | 1755_i_at | 208367_x_at | CYP3A4 |
| 160030_at | 205498_at | GHR | 1786_at | 206028_s_at | MERTK |
| 1610_s_at | 202533_s_at | DHFR /// | 178_f_at | 214473_x_at | PMS2L3 |
| | | LOC643509 /// | | | |
| | | LOC653874 | | | |
| 1627_at | 221715_at | MYST3 | 1794_at | 201700_at | CCND3 |
| 1747_at | 214992_s_at | DSE2 | 1795_g_at | 201700_at | CCND3 |
| 1747_at | 209831_x_at | DSE2 | 1875_f_at | 214473_x_at | PMS2L3 |
| 1749_at | 208369_s_at | GCDH | 190_at | 209959_at | NR4A3 |
| 1749_at | 203500_at | GCDH | 1915_s_at | 209189_at | FOS |
| 1750_at | 216602_s_at | FARSLA | 1945_at | 214710_s_at | CCNB1 |
| 1754_at | 201763_s_at | DAXX | 1951_at | 205572_at | ANGPT2 |
| 1761_at | 205226_at | PDGFRL | 1951_at | 211148_s_at | ANGPT2 |
| 177_at | 205203_at | PLD1 | 1954_at | 203934_at | KDR |
| 178_f_at | 214756_x_at | PMS2L1 | 2008_s_at | 211832_s_at | MDM2 |
| 178_f_at | 216525_x_at | PMS2L3 | 2039_s_at | 210105_s_at | FYN |
| 178_f_at | 214473_x_at | PMS2L3 | 2080_s_at | 207347_at | ERCC6 |
| 1875_f_at | 216525_x_at | PMS2L3 | 222_at | 201995_at | EXT1 |
| 1875_f_at | 214473_x_at | PMS2L3 | 243_g_at | 200836_s_at | MAP4 |
| 1875_f_at | 214756_x_at | PMS2L1 | 266_s_at | 216379_x_at | CD24 |
| 1880_at | 205386_s_at | MDM2 | 266_s_at | 209771_x_at | CD24 |
| 1945_at | 214710_s_at | CCNB1 | 266_s_at | 208651_x_at | CD24 |
| 1954_at | 203934_at | KDR | 284_at | 207156_at | HIST1H2AG |
| 201_s_at | 216231_s_at | B2M | 285_g_at | 207156_at | HIST1H2AG |
| 2042_s_at | 204798_at | MYB | 310_s_at | 206401_s_at | MAPT |
| 2055_s_at | 215878_at | ITGB1 | 310_s_at | 203928_x_at | MAPT |
| 2065_s_at | 208478_s_at | BAX | 31343_at | 216244_at | IL1RN |
| 2066_at | 208478_s_at | BAX | 31464_at | 216513_at | DCT |
| 2067_f_at | 208478_s_at | BAX | 31465_g_at | 216513_at | DCT |
| 242_at | 200836_s_at | MAP4 | 31478_at | 207077_at | ELA2B |
| 243_g_at | 200836_s_at | MAP4 | 31478_at | 206446_s_at | ELA2A |
| 262_at | 201196_s_at | AMD1 | 31506_s_at | 205033_s_at | DEFA1 /// DEFA3 |
| | | | | | /// LOC653600 |
| 263_g_at | 201196_s_at | AMD1 | 31523_f_at | 208527_x_at | HIST1H2BE |
| 272_at | 206326_at | GRP | 31524_f_at | 208523_x_at | HIST1H2BI |
| 273_g_at | 206326_at | GRP | 31574_i_at | 216405_at | LGALS1 |
| 307_at | 204446_s_at | ALOX5 | 31619_at | 217126_at | — |
| 310_s_at | 206401_s_at | MAPT | 31621_s_at | 216269_s_at | ELN |
| 310_s_at | 203928_x_at | MAPT | 31631_f_at | 214557_at | PTTG2 |
| 31343_at | 216244_at | IL1RN | 31663_at | 211111_at | — |
| 31382_f_at | 211682_x_at | UGT2B28 | 31723_at | 207925_at | CST5 |
| 31478_at | 207077_at | ELA2B | 31815_r_at | 204381_at | LRP3 |
| 31478_at | 206446_s_at | ELA2A | 31843_at | 207981_s_at | ESRRG |
| 31479_f_at | 216659_at | LOC647294 /// | 31854_at | 211208_s_at | CASK |
| | | LOC652593 | | | |
| 31506_s_at | 205033_s_at | DEFA1 /// DEFA3 | 31862_at | 205990_s_at | WNT5A |
| | | /// LOC653600 | | | |
| 31508_at | 201010_s_at | TXNIP | 31889_at | 206426_at | MLA |
| 31509_at | 208929_x_at | RPL13 | 31897_at | 204135_at | DOC1 |
| 31512_at | 216207_x_at | IGKV1D-13 /// | 31941_s_at | 207936_x_at | RFPL3 |
| | | LOC649876 | | | |
| 31525_s_at | 211745_x_at | HBA1 | 31941_s_at | 207227_x_at | RFPL2 |
| 31525_s_at | 204018_x_at | HBA1 /// HBA2 | 32001_s_at | 207414_s_at | PCSK6 |
| 31525_s_at | 209458_x_at | HBA1 /// HBA2 | 32004_s_at | 215329_s_at | CDC2L1 /// |
| | | | | | CDC2L2 |
| 31525_s_at | 211699_x_at | HBA1 /// HBA2 | 32028_at | 203201_at | PMM2 |
| 31525_s_at | 217414_x_at | HBA1 /// HBA2 | 32033_at | 204193_at | CHKB /// CPT1B |
| 31574_i_at | 216405_at | LGALS1 | 32045_at | 213213_at | DIDO1 |
| 31584_at | 212869_x_at | TPT1 | 32076_at | 203498_at | DSCR1L1 |
| 31600_s_at | 214756_x_at | PMS2L1 | 32138_at | 215116_s_at | DNM1 |
| 31619_at | 217126_at | — | 32146_s_at | 214726_x_at | ADD1 |
| 31631_f_at | 214557_at | PTTG2 | 32176_at | 212707_s_at | RASA4 /// |
| | | | | | FLJ21767 /// |
| | | | | | LOC648426 |
| 31663_at | 211111_at | — | 32177_s_at | 208534_s_at | RASA4 /// |
| | | | | | FLJ21767 |
| 31769_at | 207612_at | WNT8B | 32263_at | 202705_at | CCNB2 |
| 31806_at | 205666_at | FMO1 | 32267_at | 207236_at | ZNF345 |
| 31815_r_at | 204381_at | LRP3 | 32313_at | 204083_s_at | TPM2 |
| 31835_at | 206226_at | HRG | 32314_g_at | 204083_s_at | TPM2 |
| 31843_at | 207981_s_at | ESRRG | 32338_at | 216028_at | DKFZP564C152 |
| 31879_at | 212824_at | FUBP3 | 32420_at | 214655_at | GPR6 |
| 31897_at | 204135_at | DOC1 | 32521_at | 202037_s_at | SFRP1 |
| 31941_s_at | 207936_x_at | RFPL3 | 32542_at | 201540_at | FHL1 |
| 31941_s_at | 207227_x_at | RFPL2 | 32543_at | 200935_at | CALR |
| 32001_s_at | 207414_s_at | PCSK6 | 32543_at | 212953_x_at | CALR |
| 32004_s_at | 215329_s_at | CDC2L1 /// | 32556_at | 218382_s_at | U2AF2 |
| | | CDC2L2 | | | |
| 32028_at | 203201_at | PMM2 | 32571_at | 200769_s_at | MAT2A |
| 32045_at | 213213_at | DIDO1 | 32622_at | 202253_s_at | DNM2 |
| 32076_at | 203498_at | DSCR1L1 | 32642_at | 205143_at | CSPG3 |
| 32104_i_at | 212669_at | CAMK2G | 32649_at | 205255_x_at | TCF7 |
| 32138_at | 215116_s_at | DNM1 | 32668_at | 203787_at | SSBP2 |
| 32146_s_at | 214726_x_at | ADD1 | 32689_s_at | 210831_s_at | PTGER3 |
| 32176_at | 212707_s_at | RASA4 /// | 32710_at | 208213_s_at | KCB1 |
| | | FLJ21767 /// | | | |
| | | LOC648426 | | | |
| 32222_at | 212809_at | NFATC2IP | 32712_at | 210016_at | MYT1L |
| 32267_at | 207236_at | ZNF345 | 32728_at | 205257_s_at | AMPH |
| 32318_s_at | 200801_x_at | ACTB | 32758_g_at | 211318_s_at | RAE1 |
| 32318_s_at | 224594_x_at | ACTB | 32759_at | 211318_s_at | RAE1 |
| 32318_s_at | 213867_x_at | ACTB | 32780_at | 212254_s_at | DST |
| 32338_at | 216028_at | DKFZP564C152 | 32805_at | 204151_x_at | AKR1C1 |
| 32420_at | 214655_at | GPR6 | 32813_s_at | 203163_at | KATNB1 |
| 32435_at | 200029_at | RPL19 | 32826_at | 209473_at | — |
| 32435_at | 200029_at | RPL19 | 32885_f_at | 207752_x_at | PRB1 /// PRB2 |
| 32521_at | 202037_s_at | SFRP1 | 32885_f_at | 211531_x_at | PRB1 /// PRB2 |
| 32543_at | 200935_at | CALR | 32885_f_at | 210597_x_at | PRB1 /// PRB2 |
| 32561_at | 212523_s_at | KIAA0146 | 32906_at | 207254_at | SLC15A1 |
| 32571_at | 200769_s_at | MAT2A | 32935_at | 214758_at | WDR21A |
| 32577_s_at | 213951_s_at | PSMC3IP | 32971_at | 213900_at | C9orf61 |
| 32577_s_at | 205956_x_at | PSMC3IP | 32980_f_at | 208527_x_at | HIST1H2BE |
| 32622_at | 202253_s_at | DNM2 | 33015_at | 215768_at | SOX5 |
| 32642_at | 205143_at | CSPG3 | 33023_at | 214481_at | HIST1H2AM |
| 32649_at | 205255_x_at | TCF7 | 33127_at | 202998_s_at | LOXL2 |
| 32676_at | 221588_x_at | ALDH6A1 | 33170_at | 212911_at | DJC16 |
| 32676_at | 204290_s_at | ALDH6A1 | 33215_g_at | 204331_s_at | MRPS12 |
| 32689_s_at | 210831_s_at | PTGER3 | 33282_at | 203287_at | LAD1 |
| 32710_at | 208213_s_at | KCB1 | 33329_at | 206929_s_at | NFIC |
| 32712_at | 210016_at | MYT1L | 33427_s_at | 211852_s_at | ATRN |
| 32728_at | 205257_s_at | AMPH | 33435_r_at | 202710_at | BET1 |
| 32775_r_at | 202430_s_at | PLSCR1 | 33460_at | 207455_at | P2RY1 |
| 32779_s_at | 211323_s_at | ITPR1 | 33520_at | 207300_s_at | F7 |
| 32793_at | 213193_x_at | TRBV19 /// | 33527_at | 207142_at | KCNJ3 |
| | | TRBC1 | | | |
| 32794_g_at | 213193_x_at | TRBV19 /// | 33533_at | 203811_s_at | DJB4 |
| | | TRBC1 | | | |
| 32813_s_at | 203163_at | KATNB1 | 33534_at | 208394_x_at | ESM1 |
| 32817_at | 204541_at | SEC14L2 | 33536_at | 207505_at | PRKG2 |
| 32860_g_at | 200887_s_at | STAT1 | 33540_at | 216211_at | C10orf18 |
| 32885_f_at | 207752_x_at | PRB1 /// PRB2 | 33572_at | 206683_at | ZNF165 |
| 32885_f_at | 211531_x_at | PRB1 /// PRB2 | 33620_at | 208414_s_at | HOXB3 |
| 32885_f_at | 210597_x_at | PRB1 /// PRB2 | 33641_g_at | 215051_x_at | AIF1 |
| 32971_at | 213900_at | C9orf61 | 33673_r_at | 207245_at | UGT2B17 |
| 33015_at | 215768_at | SOX5 | 33690_at | 215322_at | LONRF1 |
| 33092_at | 214560_at | FPRL2 | 33698_at | 204251_s_at | CEP164 |
| 33127_at | 202998_s_at | LOXL2 | 33700_at | 204011_at | SPRY2 |
| 33153_at | 213952_s_at | ALOX5 | 33722_at | 212517_at | ATRN |
| 33166_at | 213443_at | TRADD | 33729_at | 204587_at | SLC25A14 |
| 33207_at | 221742_at | CUGBP1 | 33729_at | 211855_s_at | SLC25A14 |
| 33215_g_at | 204331_s_at | MRPS12 | 33746_at | 203013_at | ECD |
| 33243_at | 208296_x_at | TNFAIP8 | 33773_at | 205408_at | MLLT10 |
| 33329_at | 206929_s_at | NFIC | 33804_at | 203110_at | PTK2B |
| 33424_at | 201011_at | RPN1 | 33819_at | 201030_x_at | LDHB |
| 33425_at | 200990_at | TRIM28 | 33819_at | 213564_x_at | LDHB |
| 33435_r_at | 202710_at | BET1 | 33883_at | 204400_at | EFS |
| 33505_at | 206392_s_at | RARRES1 | 33883_at | 210880_s_at | EFS |
| 33515_at | 207503_at | TCP10 | 33884_s_at | 215533_s_at | UBE4B |
| 33520_at | 207300_s_at | F7 | 33884_s_at | 202316_x_at | UBE4B |
| 33527_at | 207142_at | KCNJ3 | 33892_at | 207717_s_at | PKP2 |
| 33533_at | 203811_s_at | DJB4 | 33920_at | 209190_s_at | DIAPH1 |
| 33534_at | 208394_x_at | ESM1 | 33936_at | 204417_at | GALC |
| 33540_at | 216211_at | C10orf18 | 33938_g_at | 215433_at | DPY19L1 |
| 33546_at | 213796_at | SPRR1A | 33991_g_at | 211298_s_at | ALB |
| 33586_at | 216006_at | WIRE | 33992_at | 211298_s_at | ALB |
| 33601_at | 215767_at | C2orf10 | 34016_s_at | 202805_s_at | ABCC1 |
| 33613_at | 215118_s_at | IGHG1 | 34033_s_at | 207857_at | LILRA2 |
| 33620_at | 208414_s_at | HOXB3 | 34052_at | 207346_at | STX2 |
| 33633_at | 214546_s_at | P2RY11 | 34065_at | 207676_at | ONECUT2 |
| 33641_g_at | 215051_x_at | AIF1 | 34090_at | 216065_at | — |
| 33641_g_at | 209901_x_at | AIF1 | 34096_at | 215170_s_at | CEP152 |
| 33650_at | 221780_s_at | DDX27 | 34187_at | 205228_at | RBMS2 |
| 33673_r_at | 207245_at | UGT2B17 | 34191_at | 212919_at | DCP2 |
| 33690_at | 215322_at | LONRF1 | 34226_at | 203553_s_at | MAP4K5 |
| 33698_at | 204251_s_at | CEP164 | 34227_i_at | 206007_at | PRG4 |
| 33700_at | 204011_at | SPRY2 | 34228_r_at | 206007_at | PRG4 |
| 33722_at | 212517_at | ATRN | 34243_i_at | 210306_at | L3MBTL |
| 33729_at | 204587_at | SLC25A14 | 34288_at | 212977_at | CMKOR1 |
| 33729_at | 211855_s_at | SLC25A14 | 34312_at | 212867_at | — |
| 33746_at | 203013_at | ECD | 34379_at | 212087_s_at | ERAL1 |
| 33758_f at | 206570_s_at | PSG1 /// PSG4 /// | 34385_at | 202004_x_at | SDHC /// |
| | | PSG7 /// PSG11 | | | LOC642502 |
| | | /// PSG8 | | | |
| 33766_at | 205019_s_at | VIPR1 | 34395_at | 203026_at | ZBTB5 |
| 33773_at | 205408_at | MLLT10 | 34476_r_at | 205767_at | EREG |
| 33819_at | 201030_x_at | LDHB | 34497_at | 216941_s_at | TAF1B |
| 33819_at | 213564_x_at | LDHB | 34594_at | 204761_at | USP6NL |
| 33857_at | 217830_s_at | NSFL1C | 34617_at | 210614_at | TTPA /// |
| | | | | | LOC649495 |
| 33861_at | 217798_at | CNOT2 | 34622_at | 207814_at | DEFA6 |
| 33883_at | 204400_at | EFS | 34631_at | 207327_at | EYA4 |
| 33883_at | 210880_s_at | EFS | 34647_at | 200033_at | DDX5 |
| 33884_s_at | 215533_s_at | UBE4B | 34647_at | 200033_at | DDX5 |
| 33884_s_at | 202316_x_at | UBE4B | 34699_at | 203593_at | CD2AP |
| 33891_at | 201560_at | CLIC4 | 34724_at | 202045_s_at | GRLF1 |
| 33892_at | 207717_s_at | PKP2 | 34726_at | 209530_at | CACNB3 |
| 33920_at | 209190_s_at | DIAPH1 | 34735_at | 214578_s_at | LOC651633 |
| 33936_at | 204417_at | GALC | 34735_at | 213044_at | LOC651633 |
| 33938_g_at | 215433_at | DPY19L1 | 34736_at | 214710_s_at | CCNB1 |
| 33991_g_at | 211298_s_at | ALB | 34778_at | 213909_at | LRRC15 |
| 33992_at | 211298_s_at | ALB | 34789_at | 211474_s_at | SERPINB6 |
| 34016_s_at | 202805_s_at | ABCC1 | 34820_at | 209465_x_at | PTN |
| 34033_s_at | 207857_at | LILRA2 | 34902_at | 215109_at | KIAA0492 |
| 34065_at | 207676_at | ONECUT2 | 34959_at | 206760_s_at | FCER2 |
| 34090_at | 216065_at | — | 34959_at | 206759_at | FCER2 |
| 34096_at | 215170_s_at | CEP152 | 34964_at | 214472_at | HIST1H3D |
| 34148_at | 206634_at | SIX3 | 34973_at | 210192_at | ATP8A1 |
| 34187_at | 205228_at | RBMS2 | 35005_at | 205851_at | NME6 |
| 34191_at | 212919_at | DCP2 | 35031_r_at | 215052_at | — |
| 34226_at | 203553_s_at | MAP4K5 | 35043_at | 207347_at | ERCC6 |
| 34243_i_at | 210306_at | L3MBTL | 35048_at | 206730_at | GRIA3 |
| 34257_at | 209737_at | MAGI2 | 35049_g_at | 206730_at | GRIA3 |
| 34312_at | 212867_at | — | 35057_at | 214775_at | N4BP3 |
| 34364_at | 202494_at | PPIE | 35074_at | 206734_at | JRKL |
| 34379_at | 212087_s_at | ERAL1 | 35106_at | 210642_at | CCIN |
| 34395_at | 203026_at | ZBTB5 | 35152_at | 205326_at | RAMP3 |
| 34470_at | 206715_at | TFEC | 35203_at | 212462_at | — |
| 34476_r_at | 205767_at | EREG | 35207_at | 203453_at | SCNN1A |
| 34521_at | 206249_at | MAP3K13 | 35211_at | 209632_at | PPP2R3A |
| 34594_at | 204761_at | USP6NL | 35214_at | 203343_at | UGDH |
| 34631_at | 207327_at | EYA4 | 35216_at | 204663_at | ME3 |
| 34644_at | 216231_s_at | B2M | 35224_at | 214696_at | MGC14376 |
| 34647_at | 200033_at | DDX5 | 35249_at | 205034_at | CCNE2 |
| 34647_at | 200033_at | DDX5 | 35265_at | 203172_at | FXR2 |
| 34678_at | 201798_s_at | FER1L3 | 35302_at | 208922_s_at | NXF1 |
| 34718_at | 203627_at | IGF1R | 35337_at | 201178_at | FBXO7 |
| 34724_at | 202045_s_at | GRLF1 | 35352_at | 202986_at | ARNT2 |
| 34726_at | 209530_at | CACNB3 | 35361_at | 209018_s_at | PINK1 |
| 34837_at | 212480_at | KIAA0376 | 35391_at | 206616_s_at | ADAM22 |
| 34894_r_at | 205847_at | PRSS22 | 35392_g_at | 206616_s_at | ADAM22 |
| 34902_at | 215109_at | KIAA0492 | 35394_at | 214778_at | MEGF8 |
| 34964_at | 214472_at | HIST1H3D | 35469_at | 207135_at | HTR2A |
| 34964_at | 214522_x_at | HIST1H3D | 35472_at | 210119_at | KCNJ15 |
| 34973_at | 210192_at | ATP8A1 | 35549_at | 210115_at | RPL39L |
| 35005_at | 205851_at | NME6 | 35576_f_at | 208523_x_at | HIST1H2BI |
| 35069_at | 208312_s_at | PRAMEF1 /// | 35588_at | 205928_at | ZNF443 |
| | | PRAMEF2 | | | |
| 35071_s_at | 214106_s_at | GMDS | 35614_at | 204849_at | TCFL5 |
| 35074_at | 206734_at | JRKL | 35650_at | 212717_at | PLEKHM1 |
| 35106_at | 210642_at | CCIN | 35666_at | 209730_at | SEMA3F |
| 35137_at | 205610_at | MYOM1 | 35677_at | 213528_at | C1orf156 |
| 35152_at | 205326_at | RAMP3 | 35683_at | 203956_at | MORC2 |
| 35203_at | 212462_at | — | 35683_at | 216863_s_at | MORC2 |
| 35205_at | 202757_at | COBRA1 | 35689_at | 206183_s_at | HERC3 |
| 35207_at | 203453_at | SCNN1A | 35693_at | 212552_at | HPCAL1 |
| 35211_at | 209632_at | PPP2R3A | 356_at | 202183_s_at | KIF22 |
| 35352_at | 202986_at | ARNT2 | 35744_at | 201978_s_at | KIAA0141 |
| 35361_at | 209018_s_at | PINK1 | 35755_at | 210740_s_at | ITPK1 |
| 35385_at | 210820_x_at | COQ7 | 35803_at | 212724_at | RND3 |
| 35394_at | 214778_at | MEGF8 | 35817_at | 209072_at | MBP |
| 35472_at | 210119_at | KCNJ15 | 35859_f_at | 214473_x_at | PMS2L3 |
| 35549_at | 210115_at | RPL39L | 35933_f_at | 214473_x_at | PMS2L3 |
| 35614_at | 204849_at | TCFL5 | 35938_at | 210145_at | PLA2G4A |
| 35677_at | 213528_at | C1orf156 | 35988_i_at | 221820_s_at | MYST1 |
| 35698_at | 203854_at | CFI | 35995_at | 204026_s_at | ZWINT |
| 35744_at | 201978_s_at | KIAA0141 | 36004_at | 209929_s_at | IKBKG |
| 35755_at | 210740_s_at | ITPK1 | 36037_g_at | 208416_s_at | SPTB |
| 35859_f_at | 214473_x_at | PMS2L3 | 36043_at | 214111_at | OPCML |
| 35859_f_at | 216525_x_at | PMS2L3 | 36057_at | 203404_at | ARMCX2 |
| 35907_at | 204826_at | CCNF | 36059_at | 212850_s_at | LRP4 |
| 35926_s_at | 213975_s_at | LYZ /// LILRB1 | 36061_at | 213169_at | — |
| 35927_r_at | 213975_s_at | LYZ /// LILRB1 | 36066_at | 212814_at | KIAA0828 |
| 35933_f_at | 216525_x_at | PMS2L3 | 36067_at | 210072_at | CCL19 |
| 35933_f_at | 214473_x_at | PMS2L3 | 36087_at | 203170_at | KIAA0409 |
| 35954_at | 206803_at | PDYN | 36103_at | 205114_s_at | CCL3 /// CCL3L1 |
| | | | | | /// CCL3L3 /// |
| | | | | | LOC643930 |
| 35988_i_at | 221820_s_at | MYST1 | 36139_at | 215411_s_at | TRAF3IP2 |
| 35995_at | 204026_s_at | ZWINT | 36146_at | 201365_at | OAZ2 |
| 36004_at | 209929_s_at | IKBKG | 36183_at | 202676_x_at | FASTK |
| 36037_g_at | 208416_s_at | SPTB | 36183_at | 214114_x_at | FASTK |
| 36043_at | 214111_at | OPCML | 36183_at | 210975_x_at | FASTK |
| 36052_at | 205268_s_at | ADD2 | 36214_at | 220266_s_at | KLF4 |
| 36059_at | 212850_s_at | LRP4 | 36229_at | 205707_at | IL17RA |
| 36061_at | 213169_at | — | 36272_r_at | 206826_at | PMP2 |
| 36066_at | 212814_at | KIAA0828 | 36347_f_at | 208527_x_at | HIST1H2BE |
| 36067_at | 210072_at | CCL19 | 36374_at | 215304_at | — |
| 36079_at | 210609_s_at | TP53I3 | 36412_s_at | 208436_s_at | IRF7 |
| 36083_at | 203227_s_at | TSPAN31 | 36451_at | 213198_at | ACVR1B |
| 36103_at | 205114_s_at | CCL3 /// CCL3L1 | 36452_at | 202796_at | SYNPO |
| | | /// CCL3L3 /// | | | |
| | | LOC643930 | | | |
| 36139_at | 215411_s_at | TRAF3IP2 | 36459_at | 204161_s_at | ENPP4 |
| 36144_at | 209197_at | SYT11 | 36577_at | 209210_s_at | PLEKHC1 |
| 36146_at | 201365_at | OAZ2 | 36607_at | 202944_at | GA |
| 36151_at | 201050_at | PLD3 | 36658_at | 200862_at | DHCR24 |
| 36191_at | 203177_x_at | TFAM | 36669_at | 202768_at | FOSB |
| 36214_at | 220266_s_at | KLF4 | 36685_at | 201197_at | AMD1 |
| 36229_at | 205707_at | IL17RA | 36711_at | 205193_at | MAFF |
| 36256_at | 214460_at | LSAMP | 36735_f_at | 216907_x_at | KIR3DL2 |
| 36272_r_at | 206826_at | PMP2 | 36739_at | 205960_at | PDK4 |
| 36318_at | 206376_at | SLC6A15 | 36746_s_at | 207886_s_at | CALCR |
| 36326_at | 215228_at | NHLH2 | 36751_at | 206107_at | RGS11 |
| 36374_at | 215304_at | — | 36757_at | 206110_at | HIST1H3H |
| 36412_s_at | 208436_s_at | IRF7 | 36782_s_at | 202410_x_at | IGF2 |
| 36451_at | 213198_at | ACVR1B | 36782_s_at | 210881_s_at | IGF2 |
| 36452_at | 202796_at | SYNPO | 36825_at | 213293_s_at | TRIM22 |
| 36459_at | 204161_s_at | ENPP4 | 36858_at | 209567_at | RRS1 |
| 36460_at | 209317_at | POLR1C | 36861_at | 209596_at | MXRA5 |
| 36462_at | 209516_at | SMYD5 | 36915_at | 203758_at | CTSO |
| 36551_at | 213701_at | C12orf29 | 36917_at | 213519_s_at | LAMA2 |
| 36600_at | 200814_at | PSME1 | 36917_at | 216840_s_at | LAMA2 |
| 36621_at | 204551_s_at | AHSG | 36970_at | 212056_at | KIAA0182 |
| 36627_at | 200795_at | SPARCL1 | 37011_at | 215051_x_at | AIF1 |
| 36735_f_at | 216907_x_at | KIR3DL2 | 37013_at | 209749_s_at | ACE |
| 36746_s_at | 207886_s_at | CALCR | 37022_at | 204223_at | PRELP |
| 36748_at | 210315_at | SYN2 | 37088_at | 211107_s_at | AURKC |
| 36782_s_at | 202410_x_at | IGF2 | 37098_at | 204788_s_at | PPOX |
| 36782_s_at | 210881_s_at | IGF2 | 37103_at | 214068_at | BEAN |
| 36790_at | 210987_x_at | TPM1 | 37124_i_at | 205765_at | CYP3A5 |
| 36791_g_at | 210987_x_at | TPM1 | 37156_at | 221911_at | ETV1 |
| 36792_at | 210986_s_at | TPM1 | 37161_at | 213750_at | — |
| 36825_at | 213293_s_at | TRIM22 | 37162_at | 204716_at | CCDC6 |
| 36861_at | 209596_at | MXRA5 | 37163_at | 213497_at | ABTB2 |
| 36890_at | 203407_at | PPL | 37164_at | 210429_at | RHD |
| 36915_at | 203758_at | CTSO | 37192_at | 204505_s_at | EPB49 |
| 36917_at | 213519_s_at | LAMA2 | 37205_at | 213249_at | FBXL7 |
| 36917_at | 216840_s_at | LAMA2 | 37260_at | 208562_s_at | ABCC9 |
| 36942_at | 200851_s_at | KIAA0174 | 37260_at | 208561_at | ABCC9 |
| 36970_at | 212056_at | KIAA0182 | 37264_at | 214741_at | ZNF131 |
| 37011_at | 209901_x_at | AIF1 | 37264_at | 221842_s_at | ZNF131 |
| 37011_at | 215051_x_at | AIF1 | 37281_at | 202771_at | FAM38A |
| 37022_at | 204223_at | PRELP | 37322_s_at | 211549_s_at | HPGD |
| 37043_at | 207826_s_at | ID3 | 37353_g_at | 202864_s_at | SP100 |
| 37088_at | 211107_s_at | AURKC | 37353_g_at | 202863_at | SP100 |
| 37098_at | 204788_s_at | PPOX | 37356_r_at | 201832_s_at | VDP |
| 37103_at | 214068_at | BEAN | 37407_s_at | 207961_x_at | MYH11 |
| 37124_i_at | 205765_at | CYP3A5 | 37423_at | 204404_at | SLC12A2 |
| 37156_at | 221911_at | ETV1 | 37457_at | 206408_at | LRRTM2 |
| 37161_at | 213750_at | — | 37469_at | 206316_s_at | KNTC1 |
| 37162_at | 204716_at | CCDC6 | 37519_at | 206743_s_at | ASGR1 |
| 37163_at | 213497_at | ABTB2 | 37548_at | 216239_at | PTHB1 |
| 37189_at | 203467_at | PMM1 | 37549_g_at | 216239_at | PTHB1 |
| 37192_at | 204505_s_at | EPB49 | 37561_at | 204108_at | NFYA |
| 37237_at | 203410_at | AP3M2 | 37565_at | 203414_at | MMD |
| 37238_s_at | 204267_x_at | PKMYT1 | 37630_at | 209763_at | CHRDL1 |
| 37260_at | 208562_s_at | ABCC9 | 37635_at | 213780_at | TCHH |
| 37260_at | 208561_at | ABCC9 | 37690_at | 202993_at | ILVBL |
| 37264_at | 214741_at | ZNF131 | 37690_at | 210624_s_at | ILVBL |
| 37264_at | 221842_s_at | ZNF131 | 37709_at | 203974_at | HDHD1A |
| 37281_at | 202771_at | FAM38A | 37721_at | 207831_x_at | DHPS |
| 37322_s_at | 211549_s_at | HPGD | 37722_s_at | 207831_x_at | DHPS |
| 37335_at | 203816_at | DGUOK | 37762_at | 201324_at | EMP1 |
| 37335_at | 209549_s_at | DGUOK | 37762_at | 201325_s_at | EMP1 |
| 37347_at | 201897_s_at | CKS1B | 37828_at | 213694_at | RSBN1 |
| 37356_r_at | 201832_s_at | VDP | 37835_at | 205987_at | CD1C |
| 37415_at | 214070_s_at | ATP10B | 37874_at | 205776_at | FMO5 |
| 37423_at | 204404_at | SLC12A2 | 37919_at | 204368_at | SLCO2A1 |
| 37449_i_at | 214548_x_at | GS | 37939_at | 209584_x_at | APOBEC3C |
| 37449_i_at | 200780_x_at | GS | 37960_at | 203921_at | CHST2 |
| 37449_i_at | 212273_x_at | GS | 37963_at | 204443_at | ARSA |
| 37449_i_at | 200981_x_at | GS | 38004_at | 214297_at | CSPG4 |
| 37450_r_at | 214548_x_at | GS | 38004_at | 204736_s_at | CSPG4 |
| 37450_r_at | 200780_x_at | GS | 38044_at | 209074_s_at | FAM107A |
| 37450_r_at | 212273_x_at | GS | 38099_r_at | 202422_s_at | ACSL4 |
| 37450_r_at | 200981_x_at | GS | 38139_at | 205140_at | FPGT |
| 37458_at | 204126_s_at | CDC45L | 38150_at | 204956_at | MTAP |
| 37469_at | 206316_s_at | KNTC1 | 38153_at | 204884_s_at | HUS1 |
| 37498_at | 214595_at | KCNG1 | 38158_at | 204817_at | ESPL1 |
| 37548_at | 216239_at | PTHB1 | 38169_s_at | 207626_s_at | SLC7A2 |
| 37549_g_at | 216239_at | PTHB1 | 38181_at | 203878_s_at | MMP11 |
| 37565_at | 203414_at | MMD | 38195_at | 204525_at | PHF14 |
| 37686_s_at | 202330_s_at | UNG | 38249_at | 215729_s_at | VGLL1 |
| 37690_at | 202993_at | ILVBL | 38256_s_at | 213794_s_at | C14orf120 |
| 37690_at | 210624_s_at | ILVBL | 38257_at | 203190_at | NDUFS8 |
| 37709_at | 203974_at | HDHD1A | 38257_at | 203189_s_at | NDUFS8 |
| 37721_at | 211558_s_at | DHPS | 38262_at | 213288_at | — |
| 37722_s_at | 211558_s_at | DHPS | 38277_at | 209817_at | PPP3CB |
| 37762_at | 201324_at | EMP1 | 38281_at | 207181_s_at | CASP7 |
| 37762_at | 201325_s_at | EMP1 | 38323_at | 208146_s_at | CPVL |
| 37765_at | 203766_s_at | LMOD1 | 38342_at | 212660_at | PHF15 |
| 37814_g_at | 214968_at | DDX51 | 38391_at | 201850_at | CAPG |
| 37828_at | 213694_at | RSBN1 | 38394_at | 212510_at | GPD1L |
| 37835_at | 205987_at | CD1C | 38414_at | 202870_s_at | CDC20 |
| 37874_at | 205776_at | FMO5 | 38445_at | 203055_s_at | ARHGEF1 |
| 37887_at | 210416_s_at | CHEK2 | 38449_at | 201886_at | WDR23 |
| 37919_at | 204368_at | SLCO2A1 | 38453_at | 204683_at | ICAM2 |
| 37937_at | 203866_at | NLE1 | 38454_g_at | 213620_s_at | ICAM2 |
| 37939_at | 209584_x_at | APOBEC3C | 38454_g_at | 204683_at | ICAM2 |
| 37969_at | 205127_at | PTGS1 | 38466_at | 202450_s_at | CTSK |
| 37992_s_at | 203926_x_at | ATP5D | 38477_at | 202632_at | DPH1 /// OVCA2 |
| 37993_at | 203926_x_at | ATP5D | 38510_at | 213817_at | — |
| 38000_at | 204476_s_at | PC | 38535_at | 208216_at | DLX4 |
| 38047_at | 209487_at | RBPMS | 38546_at | 205227_at | IL1RAP |
| 38052_at | 203305_at | F13A1 | 38574_at | 213353_at | ABCA5 |
| 38068_at | 202203_s_at | AMFR | 38576_at | 209911_x_at | HIST1H2BD |
| 38079_at | 212294_at | GNG12 | 38625_g_at | 209402_s_at | SLC12A4 |
| 38089_at | 201377_at | UBAP2L | 38625_g_at | 211112_at | SLC12A4 |
| 38105_at | 202302_s_at | FLJ11021 | 38628_at | 202182_at | GCN5L2 |
| 38139_at | 205140_at | FPGT | 38637_at | 215446_s_at | LOX |
| 38150_at | 204956_at | MTAP | 38666_at | 202880_s_at | PSCD1 |
| 38153_at | 204884_s_at | HUS1 | 38674_at | 213233_s_at | KLHL9 |
| 38169_s_at | 207626_s_at | SLC7A2 | 38721_at | 209002_s_at | CALCOCO1 |
| 38192_at | 204576_s_at | CLUAP1 | 38723_at | 209450_at | OSGEP |
| 38194_s_at | 214836_x_at | IGKC /// IGKV1-5 | 38743_f_at | 201244_s_at | RAF1 |
| 38249_at | 215729_s_at | VGLL1 | 38752_r_at | 209492_x_at | ATP5I |
| 38254_at | 212956_at | TBC1D9 | 38752_r_at | 207335_x_at | ATP5I |
| 38256_s_at | 213794_s_at | C14orf120 | 38795_s_at | 214881_s_at | UBTF |
| 38262_at | 213288_at | — | 38810_at | 202455_at | HDAC5 |
| 38263_at | 214044_at | — | 38816_at | 202289_s_at | TACC2 |
| 38271_at | 204225_at | HDAC4 | 38816_at | 211382_s_at | TACC2 |
| 38281_at | 207181_s_at | CASP7 | 38847_at | 204825_at | MELK |
| 38323_at | 208146_s_at | CPVL | 38858_at | 205262_at | KCNH2 |
| 38342_at | 212660_at | PHF15 | 38875_r_at | 205862_at | GREB1 |
| 38368_at | 209932_s_at | DUT | 38883_at | 217615_at | LRRC37A |
| 38434_at | 201511_at | AAMP | 38915_at | 206088_at | LOC474170 |
| 38449_at | 201886_at | WDR23 | 38976_at | 209083_at | CORO1A |
| 38453_at | 204683_at | ICAM2 | 38982_at | 201174_s_at | TERF2IP |
| 38454_g_at | 213620_s_at | ICAM2 | 39053_at | 202251_at | PRPF3 |
| 38454_g_at | 204683_at | ICAM2 | 39064_at | 203433_at | MTHFS |
| 38487_at | 204150_at | STAB1 | 39070_at | 201564_s_at | FSCN1 |
| 38510_at | 213817_at | — | 39070_at | 210933_s_at | FSCN1 |
| 38543_at | 208211_s_at | ALK | 39086_g_at | 202591_s_at | SSBP1 |
| 38543_at | 208212_s_at | ALK | 39103_s_at | 213279_at | DHRS1 |
| 38546_at | 205227_at | IL1RAP | 39111_s_at | 217407_x_at | PPIL2 |
| 38574_at | 213353_at | ABCA5 | 39111_s_at | 209299_x_at | PPIL2 |
| 38576_at | 209911_x_at | HIST1H2BD | 39111_s_at | 214986_x_at | PPIL2 |
| 38617_at | 202193_at | LIMK2 | 39111_s_at | 206063_x_at | PPIL2 |
| 38617_at | 210582_s_at | LIMK2 | 39115_at | 203368_at | CRELD1 |
| 38625_g_at | 209402_s_at | SLC12A4 | 39140_at | 212648_at | DHX29 |
| 38625_g_at | 211112_at | SLC12A4 | 39224_at | 213618_at | CENTD1 |
| 38637_at | 215446_s_at | LOX | 39284_at | 205800_at | SLC3A1 |
| 38646_s_at | 209752_at | REG1A | 39306_at | 208165_s_at | PRSS16 |
| 38665_at | 210701_at | CFDP1 | 39309_at | 218175_at | CCDC92 |
| 38666_at | 202880_s_at | PSCD1 | 39319_at | 205270_s_at | LCP2 |
| 38674_at | 213233_s_at | KLHL9 | 39319_at | 205269_at | LCP2 |
| 38721_at | 209002_s_at | CALCOCO1 | 39332_at | 214023_x_at | TUBB2B |
| 38723_at | 209450_at | OSGEP | 39412_at | 202702_at | TRIM26 |
| 38729_at | 200895_s_at | FKBP4 | 39416_at | 209154_at | TAX1BP3 |
| 38749_at | 212909_at | LYPD1 | 39416_at | 215464_s_at | TAX1BP3 |
| 38763_at | 201563_at | SORD | 39430_at | 202561_at | TNKS |
| 38795_s_at | 214881_s_at | UBTF | 39565_at | 204832_s_at | BMPR1A |
| 38810_at | 202455_at | HDAC5 | 39609_at | 208157_at | SIM2 |
| 38816_at | 202289_s_at | TACC2 | 39610_at | 205453_at | HOXB2 |
| 38816_at | 211382_s_at | TACC2 | 39629_at | 206178_at | PLA2G5 |
| 38823_s_at | 202693_s_at | STK17A | 39629_at | 215870_s_at | PLA2G5 |
| 38826_at | 212414_s_at | SEPT6 /// N-PAC | 39642_at | 213712_at | ELOVL2 |
| 38826_at | 212413_at | 6-Sep | 39677_at | 206102_at | GINS1 |
| 38858_at | 205262_at | KCNH2 | 39690_at | 209621_s_at | PDLIM3 |
| 38875_r_at | 205862_at | GREB1 | 39702_at | 203436_at | RPP30 |
| 388_at | 207105_s_at | PIK3R2 | 39704_s_at | 206074_s_at | HMGA1 |
| 38908_s_at | 208070_s_at | REV3L | 39737_at | 203326_x_at | — |
| 38915_at | 206088_at | LOC474170 | 39737_at | 213818_x_at | — |
| 38976_at | 209083_at | CORO1A | 39748_at | 212295_s_at | SLC7A1 |
| 39007_at | 201069_at | MMP2 | 39797_at | 212760_at | UBR2 |
| 39053_at | 202251_at | PRPF3 | 39845_at | 211152_s_at | HTRA2 |
| 39064_at | 203433_at | MTHFS | 39846_at | 203657_s_at | CTSF |
| 39069_at | 201792_at | AEBP1 | 39854_r_at | 212705_x_at | PNPLA2 |
| 39070_at | 210933_s_at | FSCN1 | 39885_at | 213598_at | HSA9761 |
| 39086_g_at | 202591_s_at | SSBP1 | 39897_at | 212455_at | YTHDC1 |
| 39103_s_at | 213279_at | DHRS1 | 39904_at | 214065_s_at | CIB2 |
| 39111_s_at | 217407_x_at | PPIL2 | 40023_at | 206382_s_at | BDNF |
| 39111_s_at | 209299_x_at | PPIL2 | 40090_at | 207628_s_at | WBSCR22 |
| 39111_s_at | 214986_x_at | PPIL2 | 40092_at | 201354_s_at | BAZ2A |
| 39111_s_at | 206063_x_at | PPIL2 | 40118_at | 212684_at | ZNF3 |
| 39115_at | 203368_at | CRELD1 | 40145_at | 201292_at | TOP2A |
| 39120_at | 204326_x_at | MT1X | 40148_at | 213419_at | APBB2 |
| 39120_at | 208581_x_at | MT1X | 40151_s_at | 203244_at | PEX5 |
| 39141_at | 200045_at | ABCF1 | 40194_at | 215470_at | DKFZP686M0199 |
| 39141_at | 200045_at | ABCF1 | 40203_at | 212227_x_at | EIF1 |
| 39172_at | 212500_at | C10orf22 | 40235_at | 203839_s_at | TNK2 |
| 39215_at | 206801_at | NPPB | 40322_at | 207526_s_at | IL1RL1 |
| 39224_at | 213618_at | CENTD1 | 40330_at | 205111_s_at | PLCE1 |
| 39284_at | 205800_at | SLC3A1 | 40330_at | 214159_at | PLCE1 |
| 39291_at | 205450_at | PHKA1 | 40371_at | 216924_s_at | DRD2 |
| 39332_at | 214023_x_at | TUBB2B | 40409_at | 202054_s_at | ALDH3A2 |
| 39412_at | 202702_at | TRIM26 | 40412_at | 203554_x_at | PTTG1 |
| 39416_at | 209154_at | TAX1BP3 | 40443_at | 208407_s_at | CTNND1 |
| 39503_s_at | 205493_s_at | DPYSL4 | 40480_s_at | 210105_s_at | FYN |
| 39530_at | 203370_s_at | PDLIM7 | 40522_at | 215001_s_at | GLUL |
| 39565_at | 204832_s_at | BMPR1A | 40576_f_at | 209068_at | HNRPDL |
| 39570_at | 212712_at | CAMSAP1 | 40659_at | 209959_at | NR4A3 |
| 39606_at | 211381_x_at | SPAG11 | 40674_s_at | 206858_s_at | HOXC6 |
| 39629_at | 206178_at | PLA2G5 | 40681_at | 205422_s_at | ITGBL1 |
| 39629_at | 215870_s_at | PLA2G5 | 40691_at | 204937_s_at | ZNF274 |
| 39637_at | 205097_at | SLC26A2 | 40717_at | 210074_at | CTSL2 |
| 39638_at | 205688_at | TFAP4 | 40734_r_at | 210319_x_at | MSX2 |
| 39642_at | 213712_at | ELOVL2 | 40756_at | 205129_at | NPM3 |
| 39677_at | 206102_at | GINS1 | 40775_at | 202746_at | ITM2A |
| 39704_s_at | 206074_s_at | HMGA1 | 40820_at | 217856_at | RBM8A |
| 39710_at | 201310_s_at | C5orf13 | 40823_s_at | 210555_s_at | NFATC3 |
| 39748_at | 212295_s_at | SLC7A1 | 40823_s_at | 210556_at | NFATC3 |
| 39797_at | 212760_at | UBR2 | 40856_at | 202283_at | SERPINF1 |
| 39854_r_at | 212705_x_at | PNPLA2 | 40890_at | 210386_s_at | MTX1 |
| 39885_at | 213598_at | HSA9761 | 40893_at | 202930_s_at | SUCLA2 |
| 39897_at | 212455_at | YTHDC1 | 40939_at | 205332_at | RCE1 |
| 39904_at | 214065_s_at | CIB2 | 40991_at | 213963_s_at | SAP30 |
| 39995_s_at | 210695_s_at | WWOX | 41015_at | 209799_at | PRKAA1 |
| 40023_at | 206382_s_at | BDNF | 41024_f_at | 207854_at | GYPE |
| 40118_at | 212684_at | ZNF3 | 41024_f_at | 216833_x_at | GYPB /// GYPE |
| 40124_at | 201614_s_at | RUVBL1 | 41024_f_at | 214407_x_at | GYPB |
| 40127_at | 220974_x_at | SFXN3 | 41061_at | 205425_at | HIP1 |
| 40127_at | 217226_s_at | SFXN3 | 41070_r_at | 204871_at | MTERF |
| 40148_at | 213419_at | APBB2 | 41100_at | 204950_at | CARD8 |
| 40194_at | 215470_at | DKFZP686M0199 | 41106_at | 204401_at | KCNN4 |
| 40322_at | 207526_s_at | IL1RL1 | 41107_at | 205104_at | SNPH |
| 40330_at | 205111_s_at | PLCE1 | 41110_at | 203533_s_at | CUL5 |
| 40330_at | 214159_at | PLCE1 | 41161_at | 201763_s_at | DAXX |
| 40336_at | 207813_s_at | FDXR | 41229_at | 213029_at | NFIB |
| 40409_at | 202054_s_at | ALDH3A2 | 41359_at | 209873_s_at | PKP3 |
| 40414_at | 201797_s_at | VARS | 41414_at | 204402_at | RHBDD3 |
| 40419_at | 201061_s_at | STOM | 41484_r_at | 214326_x_at | JUND |
| 40449_at | 208021_s_at | RFC1 | 41509_at | 200690_at | HSPA9B |
| 40489_at | 208871_at | ATN1 | 41549_s_at | 203300_x_at | AP1S2 |
| 40522_at | 215001_s_at | GLUL | 41562_at | 202265_at | BMI1 |
| 40537_at | 201025_at | EIF5B | 41638_at | 213483_at | PPWD1 |
| 40544_g_at | 209987_s_at | ASCL1 | 41646_at | 221508_at | TAOK3 |
| 40598_at | 213820_s_at | STARD5 | 41665_at | 203378_at | PCF11 |
| 40646_at | 205898_at | CX3CR1 | 41693_r_at | 204573_at | CROT |
| 40673_at | 205355_at | ACADSB | 41715_at | 204484_at | PIK3C2B |
| 40674_s_at | 206858_s_at | HOXC6 | 41762_at | 202406_s_at | TIAL1 |
| 40679_at | 206058_at | SLC6A12 | 41763_g_at | 202406_s_at | TIAL1 |
| 40681_at | 205422_s_at | ITGBL1 | 41816_at | 210026_s_at | CARD10 |
| 40691_at | 204937_s_at | ZNF274 | 41851_at | 213250_at | CCDC85B |
| 40734_r_at | 210319_x_at | MSX2 | 42980_at | 226912_at | ZDHHC23 |
| 40756_at | 205129_at | NPM3 | 43022_at | 224728_at | ATPAF1 |
| 40767_at | 213258_at | TFPI | 43511_s_at | 221861_at | — |
| 40775_at | 202746_at | ITM2A | 43525_at | 217721_at | — |
| 40820_at | 217856_at | RBM8A | 43579_at | 242440_at | CUGBP1 |
| 40823_s_at | 210555_s_at | NFATC3 | 43646_at | 219854_at | ZNF14 |
| 40823_s_at | 210556_at | NFATC3 | 43827_s_at | 201030_x_at | LDHB |
| 40856_at | 202283_at | SERPINF1 | 43827_s_at | 213564_x_at | LDHB |
| 40893_at | 202930_s_at | SUCLA2 | 43839_f_at | 221510_s_at | GLS |
| 40899_at | 201650_at | KRT19 | 43919_at | 226824_at | CPXM2 |
| 40939_at | 205332_at | RCE1 | 44026_at | 226350_at | CHML |
| 40991_at | 213963_s_at | SAP30 | 44060_at | 226317_at | PPP4R2 |
| 41024_f_at | 207854_at | GYPE | 440_at | 206929_s_at | NFIC |
| 41024_f_at | 216833_x_at | GYPB /// GYPE | 440_at | 213298_at | NFIC |
| 41024_f_at | 214407_x_at | GYPB | 44108_at | 211952_at | RANBP5 |
| 41044_at | 214061_at | WDR67 | 44131_s_at | 231714_s_at | AP4B1 |
| 41100_at | 204950_at | CARD8 | 44603_at | 228555_at | CAMK2D |
| 41106_at | 204401_at | KCNN4 | 44659_at | 219034_at | PARP16 |
| 41107_at | 205104_at | SNPH | 44787_s_at | 217913_at | VPS4A |
| 41110_at | 203533_s_at | CUL5 | 447_g_at | 202574_s_at | CSNK1G2 |
| 41161_at | 201763_s_at | DAXX | 44841_at | 218284_at | SMAD3 |
| 41316_s_at | 201748_s_at | SAFB | 44967_r_at | 242724_x_at | NR6A1 |
| 41321_s_at | 213297_at | RMND5B | 44973_at | 218950_at | CENTD3 |
| 41359_at | 209873_s_at | PKP3 | 44986_s_at | 218284_at | SMAD3 |
| 41484_r_at | 214326_x_at | JUND | 45114_at | 226363_at | ABCC5 |
| 41489_at | 203221_at | TLE1 | 45322_at | 225022_at | GOPC |
| 41505_r_at | 209348_s_at | MAF | 45441_r_at | 204915_s_at | SOX11 |
| 41509_at | 200690_at | HSPA9B | 45490_s_at | 226214_at | MIR16 |
| 41524_at | 202794_at | INPP1 | 45536_at | 205348_s_at | DYNC1I1 |
| 41549_s_at | 203300_x_at | AP1S2 | 45538_s_at | 218704_at | RNF43 |
| 41562_at | 202265_at | BMI1 | 45541_s_at | 227044_at | TBC1D22A |
| 41582_at | 205539_at | AVIL | 45652_at | 227812_at | TNFRSF19 |
| 41598_at | 214257_s_at | SEC22B | 45799_at | 218009_s_at | PRC1 |
| 41606_at | 202810_at | DRG1 | 45820_at | 218934_s_at | HSPB7 |
| 41638_at | 213483_at | PPWD1 | 45880_at | 223737_x_at | CHST9 |
| 41643_at | 215043_s_at | SMA3 /// SMA5 | 45880_at | 224400_s_at | CHST9 |
| 41646_at | 221508_at | TAOK3 | 46037_at | 243767_at | — |
| 41650_at | 203536_s_at | WDR39 | 46242_at | 218298_s_at | C14orf159 |
| 41665_at | 203378_at | PCF11 | 46256_at | 221769_at | SPSB3 |
| 41693_r_at | 204573_at | CROT | 46426_at | 219758_at | TTC26 |
| 41715_at | 204484_at | PIK3C2B | 47300_s_at | 219801_at | ZNF34 |
| 41809_at | 204215_at | C7orf23 | 47688_at | 240131_at | — |
| 41816_at | 210026_s_at | CARD10 | 48079_at | 226985_at | FGD5 |
| 42327_at | 233076_at | C10orf39 | 48364_at | 219089_s_at | ZNF576 |
| 42342_r_at | 242531_at | RRAGC | 48561_g_at | 221851_at | LOC90379 |
| 428_s_at | 216231_s_at | B2M | 48762_r_at | 218552_at | ECHDC2 |
| 42980_at | 226912_at | ZDHHC23 | 49111_at | 221861_at | — |
| 43046_at | 209167_at | GPM6B | 49125_at | 222810_s_at | RASAL2 |
| 43468_at | 226914_at | ARPC5L | 49173_at | 218731_s_at | VWA1 |
| 43468_at | 226915_s_at | ARPC5L | 49187_at | 218372_at | MED9 |
| 43511_s_at | 221861_at | — | 49316_at | 218704_at | RNF43 |
| 43569_at | 244586_x_at | ALS2CR19 | 49810_s_at | 237685_at | LOC339760 /// |
| | | | | | LOC651281 |
| 43579_at | 242440_at | CUGBP1 | 508_at | 201484_at | SUPT4H1 |
| 43727_at | 235665_at | PTOV1 | 50926_s_at | 219429_at | FA2H |
| 43827_s_at | 201030_x_at | LDHB | 51145_at | 226286_at | RBED1 |
| 43827_s_at | 213564_x_at | LDHB | 51318_r_at | 236002_at | RPS2 |
| 43839_f_at | 221510_s_at | GLS | 51406_at | 219507_at | RSRC1 |
| 43927_at | 218927_s_at | CHST12 | 51543_at | 222536_s_at | ZNF395 |
| 44060_at | 226317_at | PPP4R2 | 51625_at | 204495_s_at | C15orf39 |
| 440_at | 206929_s_at | NFIC | 51803_g_at | 218999_at | TMEM140 |
| 440_at | 213298_at | NFIC | 51822_at | 230780_at | — |
| 44131_s_at | 231714_s_at | AP4B1 | 51848_at | 227542_at | — |
| 44259_at | 228630_at | ZNF84 | 51850_s_at | 221860_at | HNRPL |
| 44603_at | 228555_at | CAMK2D | 51856_at | 219686_at | STK32B |
| 44615_at | 226969_at | LOC149448 | 51871_at | 219687_at | HHAT |
| 44659_at | 219034_at | PARP16 | 51936_at | 238332_at | ANKRD29 |
| 44787_s_at | 217913_at | VPS4A | 52204_at | 239574_at | ECHDC3 |
| 44967_r_at | 242724_x_at | NR6A1 | 52207_at | 220764_at | PPP4R2 |
| 44973_at | 218950_at | CENTD3 | 52327_s_at | 225688_s_at | PHLDB2 |
| 44983_at | 213193_x_at | TRBV19 /// | 52576_s_at | 218638_s_at | SPON2 |
| | | TRBC1 | | | |
| 45114_at | 226363_at | ABCC5 | 52658_at | 222088_s_at | SLC2A3 |
| 45299_at | 218001_at | MRPS2 | 526_s_at | 209805_at | PMS2 /// |
| | | | | | PMS2CL |
| 45322_at | 225022_at | GOPC | 52837_at | 221901_at | KIAA1644 |
| 45341_at | 201278_at | DAB2 | 52941_at | 221823_at | LOC90355 |
| 45342_at | 217844_at | CTDSP1 | 53122_at | 218933_at | SPATA5L1 |
| 45383_at | 203926_x_at | ATP5D | 53122_at | 222163_s_at | SPATA5L1 |
| 45385_g_at | 222597_at | SP29 | 53550_at | 236038_at | — |
| 45536_at | 205348_s_at | DYNC1I1 | 53784_at | 227894_at | KIAA1924 |
| 45538_s_at | 218704_at | RNF43 | 53835_at | 212528_at | — |
| 45541_s_at | 227044_at | TBC1D22A | 54000_at | 223203_at | TMEM29 /// |
| | | | | | LOC653094 /// |
| | | | | | LOC653504 /// |
| | | | | | LOC653507 |
| 45598_at | 219403_s_at | HPSE | 54077_at | 218888_s_at | NETO2 |
| 45652_at | 227812_at | TNFRSF19 | 54093_at | 218403_at | TRIAP1 |
| 45676_at | 218741_at | C22orf18 | 54280_at | 240555_at | MITF |
| 45799_at | 218009_s_at | PRC1 | 54420_at | 221218_s_at | TPK1 |
| 45880_at | 223737_x_at | CHST9 | 54420_at | 223686_at | TPK1 |
| 45880_at | 224400_s_at | CHST9 | 54886_at | 225688_s_at | PHLDB2 |
| 46037_at | 243767_at | — | 55013_at | 225147_at | PSCD3 |
| 46137_at | 229962_at | FLJ34306 | 55028_at | 224715_at | WDR34 |
| 46256_at | 221769_at | SPSB3 | 55117_at | 243453_at | — |
| 46290_at | 217961_at | FLJ20551 | 55150_at | 239413_at | CEP152 |
| 46295_at | 221515_s_at | LCMT1 | 55185_at | 239436_at | CHORDC1 |
| 46364_at | 236537_at | — | 55449_i_at | 229459_at | FAM19A5 |
| 46426_at | 219758_at | TTC26 | 55639_at | 215974_at | HCG4P6 |
| 46595_at | 221780_s_at | DDX27 | 55868_at | 230157_at | CDH24 |
| 46659_at | 226702_at | LOC129607 | 56126_at | 219370_at | RPRM |
| 46694_at | 218162_at | OLFML3 | 56142_r_at | 230698_at | — |
| 47088_at | 229598_at | COBLL1 | 56251_at | 212177_at | C6orf111 |
| 47110_at | 227174_at | WDR72 | 56295_at | 225075_at | PDRG1 |
| 47550_at | 219042_at | LZTS1 | 57205_at | 223007_s_at | C9orf5 |
| 47688_at | 240131_at | — | 57302_at | 206783_at | FGF4 |
| 47778_at | 230357_at | GMDS | 56401_at | 218005_at | ZNF22 |
| 47884_at | 236456_at | PTPN5 | 56712_at | 236704_at | PDE4DIP |
| 48079_at | 226985_at | FGD5 | 56812_at | 219148_at | PBK |
| 480_at | 204267_x_at | PKMYT1 | 56819_at | 230184_at | — |
| 48114_g_at | 218865_at | MOSC1 | 56870_g_at | 219222_at | RBKS |
| 48364_at | 219089_s_at | ZNF576 | 57013_s_at | 218996_at | TFPT |
| 48384_at | 229661_at | SALL4 | 57085_s_at | 215411_s_at | TRAF3IP2 |
| 48550_at | 218454_at | FLJ22662 | 57531_at | 228448_at | MAP6 |
| 48581_at | 225187_at | KIAA1967 | 57534_at | 226987_at | RBM15B |
| 49111_at | 221861_at | — | 57539_at | 221848_at | ZGPAT |
| 49125_at | 222810_s_at | RASAL2 | 57540_at | 219222_at | RBKS |
| 49161_at | 240512_x_at | KCTD4 | 57781_at | 244648_at | CCDC93 |
| 49187_at | 218372_at | MED9 | 57954_at | 225407_at | MBP |
| 49316_at | 218704_at | RNF43 | 57984_at | 236284_at | KIAA0146 |
| 49519_at | 218037_at | C2orf17 | 58082_at | 232237_at | MDGA1 |
| 49587_at | 218873_at | GON4L | 58366_at | 228694_at | — |
| 49589_g_at | 218873_at | GON4L | 583_s_at | 203868_s_at | VCAM1 |
| 49810_s_at | 237685_at | LOC339760 /// | 58622_at | 230466_s_at | RASSF3 |
| | | LOC651281 | | | |
| 49874_at | 229592_at | — | 58799_at | 229191_at | TBCD |
| 50098_at | 220979_s_at | ST6GALC5 | 58984_at | 229672_at | C20orf44 |
| 50354_at | 219117_s_at | FKBP11 | 59616_at | 229121_at | — |
| 50926_s_at | 219429_at | FA2H | 59658_at | 215731_s_at | MPHOSPH9 |
| 51092_at | 221816_s_at | PHF11 | 59658_at | 221965_at | MPHOSPH9 |
| 51145_at | 226286_at | RBED1 | 59661_at | 227614_at | HKDC1 |
| 51406_at | 219507_at | RSRC1 | 599_at | 214438_at | HLX1 |
| 51543_at | 222536_s_at | ZNF395 | 600_at | 206113_s_at | RAB5A |
| 51625_at | 204495_s_at | C15orf39 | 60199_at | 218521_s_at | UBE2W |
| 51702_at | 238649_at | PITPNC1 | 60517_at | 228717_at | PANK1 |
| 51755_at | 220107_s_at | C14orf140 | 60535_g_at | 221042_s_at | CLMN |
| 51816_at | 219078_at | GPATC2 | 61003_at | 243139_at | SV2C |
| 51822_at | 230780_at | — | 61119_at | 204039_at | CEBPA |
| 51848_at | 227542_at | — | 61274_s_at | 208772_at | ANKHD1 /// |
| | | | | | MASK-BP3 |
| 51856_at | 219686_at | STK32B | 615_s_at | 210355_at | PTHLH |
| 51871_at | 219687_at | HHAT | 61659_at | 227188_at | C21orf63 |
| 51936_at | 238332_at | ANKRD29 | 62210_at | 218996_at | TFPT |
| 52170_at | 204037_at | EDG2 /// | 63325_at | 221860_at | HNRPL |
| | | LOC644923 | | | |
| 52204_at | 239574_at | ECHDC3 | 63361_at | 218638_s_at | SPON2 |
| 52327_s_at | 225688_s_at | PHLDB2 | 63388_at | 200856_x_at | NCOR1 /// |
| | | | | | C20orf191 |
| 52574_at | 243424_at | SOX6 | 63872_g_at | 218552_at | ECHDC2 |
| 52720_r_at | 236705_at | MGC42090 | 64184_at | 219596_at | THAP10 |
| 52837_at | 221901_at | KIAA1644 | 64339_s_at | 218636_s_at | MAN1B1 |
| 52941_at | 221823_at | LOC90355 | 64364_at | 201354_s_at | BAZ2A |
| 53122_at | 218933_at | SPATA5L1 | 64475_at | 221447_s_at | GLT8D2 |
| 53122_at | 222163_s_at | SPATA5L1 | 64489_at | 218039_at | NUSAP1 |
| 53550_at | 236038_at | — | 65079_at | 226668_at | WDSUB1 |
| 53714_at | 222540_s_at | RSF1 | 65492_at | 225835_at | SLC12A2 |
| 53784_at | 227894_at | KIAA1924 | 65720_at | 218418_s_at | ANKRD25 |
| 53835_at | 212528_at | — | 65884_at | 218636_s_at | MAN1B1 |
| 53911_at | 218220_at | C12orf10 | 65983_at | 218284_at | SMAD3 |
| 53968_at | 221818_at | INTS5 | 66148_i_at | 244231_at | — |
| 54000_at | 223203_at | TMEM29 /// | 679_at | 205653_at | CTSG |
| | | LOC653094 /// | | | |
| | | LOC653504 /// | | | |
| | | LOC653507 | | | |
| 54280_at | 240555_at | MITF | 69680_at | 207445_s_at | CCR9 |
| 54420_at | 221218_s_at | TPK1 | 71949_at | 202903_at | LSM5 |
| 54420_at | 223686_at | TPK1 | 72441_at | 202885_s_at | PPP2R1B |
| 54886_at | 225688_s_at | PHLDB2 | 744_at | 203334_at | DHX8 |
| 55009_at | 224452_s_at | MGC12966 | 76343_at | 218658_s_at | ACTR8 |
| 55013_at | 225147_at | PSCD3 | 767_at | 207961_x_at | MYH11 |
| 55026_at | 219142_at | RASL11B | 773_at | 201496_x_at | MYH11 |
| 55093_at | 221799_at | CSGlcA-T | 774_g_at | 201496_x_at | MYH11 |
| 55117_at | 243453_at | — | 78359_at | 219125_s_at | RAG1AP1 |
| 55150_at | 239413_at | CEP152 | 78684_at | 212230_at | PPAP2B |
| 55185_at | 239436_at | CHORDC1 | 80446_at | 204883_s_at | HUS1 |
| 55449_i_at | 229459_at | FAM19A5 | 80572_at | 201540_at | FHL1 |
| 55469_at | 205521_at | ENDOGL1 | 806_at | 204958_at | PLK3 |
| 55650_at | 218656_s_at | LHFP | 809_at | 209514_s_at | RAB27A |
| 55798_at | 218775_s_at | WWC2 | 809_at | 210951_x_at | RAB27A |
| 55806_at | 235430_at | C14orf43 | 823_at | 203687_at | CX3CL1 |
| 55853_at | 219923_at | TRIM45 | 828_at | 206631_at | PTGER2 |
| 55912_at | 218534_s_at | AGGF1 | 829_s_at | 200824_at | GSTP1 |
| 56126_at | 219370_at | RPRM | 83193_at | 222073_at | COL4A3 |
| 56142_r_at | 230698_at | — | 85141_at | 202970_at | — |
| 56251_at | 212177_at | C6orf111 | 85822_at | 219797_at | MGAT4A |
| 56295_at | 225075_at | PDRG1 | 873_at | 213844_at | HOXA5 |
| 56305_at | 219316_s_at | C14orf58 | 877_at | 204314_s_at | CREB1 |
| 57205_at | 223007_s_at | C9orf5 | 877_at | 204313_s_at | CREB1 |
| 57272_at | 210695_s_at | WWOX | 88242_at | 209527_at | EXOSC2 |
| 57404_at | 241224_x_at | DSCR8 | 89217_at | 213722_at | SOX2 |
| 56409_at | 218087_s_at | SORBS1 | 89799_at | 219997_s_at | COPS7B |
| 56504_at | 218584_at | FLJ21127 | 89919_s_at | 209154_at | TAX1BP3 |
| 56712_at | 236704_at | PDE4DIP | 89919_s_at | 215464_s_at | TAX1BP3 |
| 56967_at | 219606_at | PHF20L1 | 90412_i_at | 219538_at | WDR5B |
| 57085_s_at | 215411_s_at | TRAF3IP2 | 90414_f_at | 219538_at | WDR5B |
| 57516_at | 222120_at | MGC13138 | 90695_at | 222307_at | LOC282997 |
| 57567_at | 226031_at | FLJ20097 | 91099_i_at | 214695_at | UBAP2L |
| 57684_at | 221049_s_at | POLL | 91101_r_at | 214695_at | UBAP2L |
| 57718_at | 224694_at | ANTXR1 | 91137_at | 214695_at | UBAP2L |
| 57755_at | 231165_at | DDHD1 | 914_g_at | 211626_x_at | ERG |
| 57781_at | 244648_at | CCDC93 | 914_g_at | 213541_s_at | ERG |
| 57839_g_at | 220788_s_at | RNF31 | 993_at | 205546_s_at | TYK2 |
| 57954_at | 225407_at | MBP | | 200784_s_at | LRP1 |
| 58082_at | 232237_at | MDGA1 | | 200923_at | LGALS3BP |
| 58329_at | 218944_at | PYCRL | | 201044_x_at | DUSP1 |
| 58356_at | 219100_at | OBFC1 | | 201169_s_at | BHLHB2 |
| 58366_at | 228694_at | — | | 201208_s_at | TNFAIP1 |
| 58472_f_at | 238570_at | — | | 201297_s_at | MOBK1B |
| 58589_s_at | 214460_at | LSAMP | | 201367_s_at | ZFP36L2 |
| 58622_at | 230466_s_at | RASSF3 | | 201371_s_at | CUL3 |
| 58666_at | 242178_at | LIPI | | 201685_s_at | C14orf92 |
| 58798_at | 201590_x_at | ANXA2 | | 201739_at | SGK |
| 58799_at | 229191_at | TBCD | | 201793_x_at | SMG7 |
| 58984_at | 229672_at | C20orf44 | | 201796_s_at | VARS |
| 59038_at | 228784_at | ST3GAL2 | | 202186_x_at | PPP2R5A |
| 59616_at | 229121_at | — | | 202358_s_at | SNX19 |
| 59658_at | 215731_s_at | MPHOSPH9 | | 202924_s_at | PLAGL2 |
| 59658_at | 221965_at | MPHOSPH9 | | 202935_s_at | SOX9 |
| 59661_at | 227614_at | HKDC1 | | 203383_s_at | GOLGA1 |
| 59719_at | 229191_at | TBCD | | 203479_s_at | OTUD4 |
| 59766_at | 230640_at | PRPF40B | | 203597_s_at | WBP4 |
| 599_at | 214438_at | HLX1 | | 204298_s_at | LOX |
| 60034_at | 226360_at | ZNRF3 | | 205625_s_at | CALB1 |
| 600_at | 206113_s_at | RAB5A | | 205915_x_at | GRIN1 |
| 60517_at | 228717_at | PANK1 | | 207045_at | FLJ20097 |
| 60535_g_at | 221042_s_at | CLMN | | 207331_at | CENPF |
| 61003_at | 243139_at | SV2C | | 207465_at | — |
| 61119_at | 204039_at | CEBPA | | 207746_at | POLQ |
| 61274_s_at | 208772_at | ANKHD1 /// | | 207902_at | IL5RA |
| | | MASK-BP3 | | | |
| 61342_at | 227934_at | — | | 208144_s_at | — |
| 61538_r_at | 214600_at | TEAD1 | | 208461_at | HIC1 |
| 615_s_at | 210355_at | PTHLH | | 208504_x_at | PCDHB11 |
| 61931_at | 228270_at | DKFZp434J1015 | | 208545_x_at | TAF4 |
| | | /// | | | |
| | | DKFZp547K054 | | | |
| 61931_at | 232884_s_at | DKFZp434J1015 | | 208583_x_at | HIST1H2AJ |
| 62940_f_at | 221872_at | RARRES1 | | 209034_at | PNRC1 |
| 62941_r_at | 221872_at | RARRES1 | | 209052_s_at | WHSC1 |
| 63361_at | 218638_s_at | SPON2 | | 209053_s_at | WHSC1 |
| 63388_at | 200856_x_at | NCOR1 /// | | 209078_s_at | TXN2 |
| | | C20orf191 | | | |
| 63396_at | 222258_s_at | SH3BP4 | | 209368_at | EPHX2 |
| 634_at | 202525_at | PRSS8 | | 209677_at | PRKCI |
| 63883_at | 222130_s_at | FTSJ2 | | 210197_at | ITPK1 |
| 639_s_at | 202819_s_at | TCEB3 | | 210245_at | ABCC8 |
| 64006_s_at | 218656_s_at | LHFP | | 210256_s_at | PIP5K1A |
| 64048_at | 218396_at | VPS13C | | 210572_at | PCDHA2 |
| 64145_at | 218741_at | C22orf18 | | 210712_at | LDHAL6B |
| 64292_s_at | 218312_s_at | ZNF447 | | 211001_at | TRIM29 |
| 64339_s_at | 218636_s_at | MAN1B1 | | 211077_s_at | TLK1 |
| 64526_at | 220595_at | PDZRN4 | | 211127_x_at | EDA |
| 64881_at | 219986_s_at | ACAD10 | | 211304_x_at | KCNJ5 |
| 649_s_at | 217028_at | CXCR4 | | 211310_at | EZH1 |
| 65079_at | 226668_at | WDSUB1 | | 211337_s_at | 76P |
| 65443_at | 218272_at | FLJ20699 | | 211427_s_at | KCNJ13 |
| 65484_f_at | 221510_s_at | GLS | | 211502_s_at | PFTK1 |
| 65492_at | 225835_at | SLC12A2 | | 211520_s_at | GRIA1 |
| 65604_at | 218730_s_at | OGN | | 211572_s_at | SLC23A2 |
| 65613_at | 218331_s_at | C10orf18 | | 211731_x_at | SSX3 |
| 656_at | 202794_at | INPP1 | | 211776_s_at | EPB41L3 |
| 65710_at | 217832_at | SYNCRIP | | 211864_s_at | FER1L3 |
| 65884_at | 218636_s_at | MAN1B1 | | 212283_at | AGRN |
| 66148_i_at | 244231_at | — | | 212743_at | RCHY1 |
| 668_s_at | 204259_at | MMP7 | | 212862_at | CDS2 |
| 669_s_at | 202531_at | IRF1 | | 213006_at | CEBPD |
| 671_at | 200665_s_at | SPARC | | 213274_s_at | CTSB |
| 675_at | 214022_s_at | IFITM1 | | 213328_at | NEK1 |
| 675_at | 201601_x_at | IFITM1 | | 213772_s_at | GGA2 |
| 676_g_at | 214022_s_at | IFITM1 | | 214250_at | NUMA1 |
| 676_g_at | 201601_x_at | IFITM1 | | 214283_at | TMEM97 |
| 679_at | 205653_at | CTSG | | 214366_s_at | ALOX5 |
| 73236_g_at | 202269_x_at | GBP1 | | 214842_s_at | ALB |
| 740_at | 216615_s_at | HTR3A | | 215103_at | CYP2C18 |
| 740_at | 217002_s_at | HTR3A | | 215198_s_at | CALD1 |
| 744_at | 203334_at | DHX8 | | 215249_at | RPL35A |
| 74576_at | 219660_s_at | ATP8A2 | | 215531_s_at | GABRA5 /// |
| | | | | | LOC653222 |
| 74779_s_at | 205666_at | FMO1 | | 215560_x_at | MTRF1L |
| 74932_at | 202333_s_at | UBE2B | | 215611_at | TCF12 |
| 75229_at | 213732_at | TCF3 | | 215615_x_at | RERE |
| 753_at | 204114_at | NID2 | | 215637_at | TSGA14 |
| 75722_at | 219634_at | CHST11 | | 215758_x_at | ZNF93 |
| 769_s_at | 201590_x_at | ANXA2 | | 215779_s_at | HIST1H2BG |
| 77595_at | 221189_s_at | TARSL1 | | 215978_x_at | LOC152719 |
| 78107_at | 213741_s_at | KP1 | | 216002_at | FNTB |
| 78622_r_at | 218312_s_at | ZNF447 | | 216017_s_at | B2 |
| 78684_at | 212230_at | PPAP2B | | 216146_at | — |
| 78737_at | 201408_at | PPP1CB | | 216161_at | SBNO1 |
| 80446_at | 204883_s_at | HUS1 | | 216284_at | — |
| 80456_s_at | 208676_s_at | PA2G4 | | 216319_at | — |
| 806_at | 204958_at | PLK3 | | 216340_s_at | CYP2A7P1 |
| 809_at | 209514_s_at | RAB27A | | 216422_at | PA2G4 |
| 809_at | 210951_x_at | RAB27A | | 216522_at | OR2B6 |
| 81410_at | 214681_at | GK | | 216583_x_at | — |
| 820_at | 204168_at | MGST2 | | 216592_at | MAGEC3 |
| 828_at | 206631_at | PTGER2 | | 216810_at | KRTAP4-7 |
| 829_s_at | 200824_at | GSTP1 | | 216860_s_at | GDF11 |
| 83413_at | 231432_at | GRP | | 216928_at | TAL1 |
| 85141_at | 202970_at | — | | 217112_at | PDGFB |
| 873_at | 213844_at | HOXA5 | | 217136_at | PPIAL4 /// |
| | | | | | LOC653505 /// |
| | | | | | LOC653598 |
| 877_at | 204314_s_at | CREB1 | | 217362_x_at | HLA-DRB6 |
| 877_at | 204313_s_at | CREB1 | | 217612_at | TIMM50 |
| 87833_at | 213732_at | TCF3 | | 218182_s_at | CLDN1 |
| 881_at | 208083_s_at | ITGB6 | | 218564_at | RFWD3 |
| 881_at | 208084_at | ITGB6 | | 218621_at | HEMK1 |
| 89799_at | 219997_s_at | COPS7B | | 218744_s_at | PACSIN3 |
| 89882_at | 214022_s_at | IFITM1 | | 220444_at | ZNF557 |
| 89898_at | 222006_at | LETM1 | | 220549_at | RAD54B |
| 89919_s_at | 209154_at | TAX1BP3 | | 220631_at | OSGEPL1 |
| 89960_at | 202333_s_at | UBE2B | | 220791_x_at | SCN11A |
| 90410_at | 219055_at | SRBD1 | | 221358_at | NPBWR2 |
| 90695_at | 222307_at | LOC282997 | | 221409_at | OR2S2 |
| 914_g_at | 211626_x_at | ERG | | 221595_at | — |
| 914_g_at | 213541_s_at | ERG | | 221905_at | CYLD |
| 916_at | 204945_at | PTPRN | | 222038_s_at | UTP18 |
| 917_g_at | 204945_at | PTPRN | | 222184_at | — |
| | 1552286_at | ATP6V1E2 | | 222264_at | HNRPUL2 |
| | 1557372_at | ATP6V1E2 | | 31845_at | ELF4 |
| | 1561574_at | SLIT3 | | 35776_at | ITSN1 |
| | 201060_x_at | STOM | | 40359_at | RASSF7 |
| | 201137_s_at | HLA-DPB1 | | 52651_at | COL8A2 |
| | 201309_x_at | C5orf13 | | 65884_at | MAN1B1 |
| | 201793_x_at | SMG7 | | 52651_at | COL8A2 |
| | 201796_s_at | VARS | | 65884_at | MAN1B1 |
| | 201905_s_at | CTDSPL | | | |
| | 202255_s_at | SIPA1L1 | | | |
| | 202291_s_at | MGP | | | |
| | 202358_s_at | SNX19 | | | |
| | 202472_at | MPI | | | |
| | 202897_at | SIRPA | | | |
| | 202935_s_at | SOX9 | | | |
| | 203290_at | HLA-DQA1 | | | |
| | 203398_s_at | GALNT3 | | | |
| | 203532_x_at | CUL5 | | | |
| | 203705_s_at | FZD7 | | | |
| | 203793_x_at | PCGF2 | | | |
| | 203810_at | DJB4 | | | |
| | 203813_s_at | SLIT3 | | | |
| | 204036_at | EDG2 | | | |
| | 204111_at | HNMT | | | |
| | 204222_s_at | GLIPR1 | | | |
| | 204298_s_at | LOX | | | |
| | 204364_s_at | REEP1 | | | |
| | 204514_at | DPH2 | | | |
| | 204939_s_at | PLN | | | |
| | 205158_at | RSE4 | | | |
| | 205371_s_at | DBT | | | |
| | 205625_s_at | CALB1 | | | |
| | 206389_s_at | PDE3A | | | |
| | 207511_s_at | C2orf24 | | | |
| | 207772_s_at | PRMT8 | | | |
| | 207797_s_at | LRP2BP | | | |
| | 208180_s_at | HIST1H4H | | | |
| | 208504_x_at | PCDHB11 | | | |
| | 209034_at | PNRC1 | | | |
| | 209053_s_at | WHSC1 | | | |
| | 209078_s_at | TXN2 | | | |
| | 209168_at | GPM6B | | | |
| | 209247_s_at | ABCF2 | | | |
| | 209288_s_at | CDC42EP3 | | | |
| | 209291_at | ID4 | | | |
| | 209423_s_at | PHF20 | | | |
| | 209500_x_at | TNFSF13 /// | | | |
| | | TNFSF12- | | | |
| | | TNFSF13 | | | |
| | 209658_at | CDC16 | | | |
| | 209802_at | PHLDA2 | | | |
| | 210132_at | EF3 | | | |
| | 210256_s_at | PIP5K1A | | | |
| | 210314_x_at | TNFSF13 /// | | | |
| | | TNFSF12- | | | |
| | | TNFSF13 | | | |
| | 210572_at | PCDHA2 | | | |
| | 210635_s_at | KLHL20 | | | |
| | 210712_at | LDHAL6B | | | |
| | 210718_s_at | ARL17P1 | | | |
| | 210931_at | RNF6 | | | |
| | 211077_s_at | TLK1 | | | |
| | 211310_at | EZH1 | | | |
| | 211337_s_at | 76P | | | |
| | 211389_x_at | KIR3DL1 | | | |
| | 211427_s_at | KCNJ13 | | | |
| | 211520_s_at | GRIA1 | | | |
| | 211776_s_at | EPB41L3 | | | |
| | 212092_at | PEG10 | | | |
| | 212671_s_at | HLA-DQA1 /// | | | |
| | | HLA-DQA2 /// | | | |
| | | LOC650946 | | | |
| | 212743_at | RCHY1 | | | |
| | 213006_at | CEBPD | | | |
| | 213490_s_at | MAP2K2 | | | |
| | 213688_at | CALM1 | | | |
| | 213957_s_at | CEP350 | | | |
| | 214252_s_at | CLN5 | | | |
| | 214283_at | TMEM97 | | | |
| | 214543_x_at | QKI | | | |
| | 214649_s_at | MTMR2 | | | |
| | 214675_at | NUP188 | | | |
| | 215187_at | FLJ11292 | | | |
| | 215198_s_at | CALD1 | | | |
| | 215468_at | LOC647070 | | | |
| | 215637_at | TSGA14 | | | |
| | 216002_at | FNTB | | | |
| | 216091_s_at | BTRC | | | |
| | 216161_at | SBNO1 | | | |
| | 216216_at | SLIT3 | | | |
| | 216315_x_at | UBE2V1 /// Kua- | | | |
| | | UEV | | | |
| | 216354_at | — | | | |
| | 216514_at | — | | | |
| | 216592_at | MAGEC3 | | | |
| | 216810_at | KRTAP4-7 | | | |
| | 216813_at | — | | | |
| | 216850_at | SNRPN | | | |
| | 216969_s_at | KIF22 | | | |
| | 217071_s_at | MTHFR | | | |
| | 217187_at | MUC5AC | | | |
| | 217209_at | — | | | |
| | 217362_x_at | HLA-DRB6 | | | |
| | 217392_at | CAPZA1 | | | |
| | 217401_at | — | | | |
| | 217448_s_at | C14orf92 | | | |
| | 217538_at | RUTBC1 | | | |
| | 217612_at | TIMM50 | | | |
| | 217618_x_at | HUS1 | | | |
| | 218182_s_at | CLDN1 | | | |
| | 218564_at | RFWD3 | | | |
| | 218589_at | P2RY5 | | | |
| | 218621_at | HEMK1 | | | |
| | 218744_s_at | PACSIN3 | | | |
| | 219451_at | MSRB2 | | | |
| | 219810_at | VCPIP1 | | | |
| | 220037_s_at | XLKD1 | | | |
| | 220564_at | C10orf59 | | | |
| | 220584_at | FLJ22184 | | | |
| | 220631_at | OSGEPL1 | | | |
| | 220789_s_at | TBRG4 | | | |
| | 220791_x_at | SCN11A | | | |
| | 220908_at | CCDC33 | | | |
| | 221356_x_at | P2RX2 | | | |
| | 221440_s_at | RBBP9 | | | |
| | 221595_at | — | | | |
| | 221683_s_at | CEP290 | | | |
| | 222038_s_at | UTP18 | | | |
| | 222141_at | KLHL22 | | | |
| | 222170_at | LOC440334 | | | |
| | 222176_at | PTEN | | | |
| | 222247_at | DXS542 | | | |
| | 34868_at | SMG5 | | | |
| | 35776_at | ITSN1 | | | |
| | 37278_at | TAZ | | | |
| | 40489_at | ATN1 | | | |
| | 53968_at | INTS5 | | | |
| | 42447_at | SLIT3 | | | |
| | | GI_3253412 | | | |
| | | GI_9120119 | | | |
| | | PRO1489 | | | |

TABLE 8B
Tissue (tumor or stroma) specific relapse related genes. Normal font: up-regulated
genes. Italics: down-regulated genes.

| | | Stroma Specific Relapse Related Genes | |
|---|---|---|---|
| Tumor Specific Relapse Related Genes | | U133 Probe | |
| U133 Probe Set ID | Gene Symbol | Set ID | Gene Symbol |
| 218312_s_at | ZNF447 | 209959_at | NR4A3 |
| 209737_at | MAGI2 | 202935_s_at | SOX9 |
| 201137_s_at | HLA-DPB1 | 201650_at | KRT19 |
| 201408_at | PPP1CB | 201496_x_at | MYH11 |
| 208180_s_at | HIST1H4H | 203453_at | SCNN1A |
| 213789_at | — | 213629_x_at | MT1F |
| 214600_at | TEAD1 | 210915_x_at | TRBV19 /// TRBC1 |
| 210314_x_at | TNFSF13 /// | 218888_s_at | NETO2 |
| | TNFSF12- | | |
| | TNFSF13 | | |
| 204384_at | GOLGA2 | 203932_at | HLA-DMB |
| 204916_at | RAMP1 | 206391_at | RARRES1 |
| 212909_at | LYPD1 | 200923_at | LGALS3BP |
| 209078_s_at | TXN2 | 201044_x_at | DUSP1 |
| 221799_at | CSGlcA-T | 213564_x_at | LDHB |
| 216450_x_at | HSP90B1 | 213746_s_at | FL |
| 205226_at | PDGFRL | 210299_s_at | FHL1 |
| 201267_s_at | PSMC3 | 218731_s_at | VWA1 |
| 220584_at | FLJ22184 | 222162_s_at | ADAMTS1 |
| 214472_at | HIST1H3D | 204135_at | DOC1 |
| 203467_at | PMM1 | 222073_at | COL4A3 |
| 202525_at | PRSS8 | 201367_s_at | ZFP36L2 |
| 200811_at | CIRBP | 202222_s_at | DES |
| 214522_x_at | HIST1H3D | 201495_x_at | MYH11 |
| 209500_x_at | TNFSF13 /// | 201030_x_at | LDHB |
| | TNFSF12- | | |
| | TNFSF13 | | |
| 211558_s_at | DHPS | 211864_s_at | FER1L3 |
| 201748_s_at | SAFB | 202269_x_at | GBP1 |
| 208490_x_at | HIST1H2BF | 205928_at | ZNF443 |
| 208579_x_at | H2BFS | 216860_s_at | GDF11 |
| 201797_s_at | VARS | 213293_s_at | TRIM22 |
| 208546_x_at | HIST1H2BH | 211417_x_at | GGT1 |
| 201101_s_at | BCLAF1 | 207826_s_at | ID3 |
| 219660_s_at | ATP8A2 | 201297_s_at | MOBK1B |
| 205750_at | BPHL | 200974_at | ACTA2 |
| 219438_at | FAM77C | 200953_s_at | CCND2 |
| 208523_x_at | HIST1H2BI | 212254_s_at | DST |
| 205371_s_at | DBT | 207961_x_at | MYH11 |
| 221742_at | CUGBP1 | 201787_at | FBLN1 |
| 202102_s_at | BRD4 | 201235_s_at | BTG2 |
| 212684_at | ZNF3 | 202283_at | SERPINF1 |
| 201897_s_at | CKS1B | 201169_s_at | BHLHB2 |
| 216354_at | — | 205383_s_at | ZBTB20 |
| 209218_at | SQLE | 210298_x_at | FHL1 |
| 214460_at | LSAMP | 222088_s_at | SLC2A3 |
| 205480_s_at | UGP2 | 210072_at | CCL19 |
| 203368_at | CRELD1 | 201540_at | FHL1 |
| 53968_at | INTS5 | 201310_s_at | C5orf13 |
| 210052_s_at | TPX2 | 211798_x_at | IGLJ3 |
| 205376_at | INPP4B | 213258_at | TFPI |
| 210410_s_at | MSH5 | 209154_at | TAX1BP3 |
| 204343_at | ABCA3 | 215016_x_at | DST |
| 211389_x_at | KIR3DL1 | 203851_at | IGFBP6 |
| 207950_s_at | ANK3 | 201484_at | SUPT4H1 |
| 209317_at | POLR1C | 214040_s_at | GSN |
| 203767_s_at | STS | 202498_s_at | SLC2A3 |
| 207156_at | HIST1H2AG | 202688_at | TNFSF10 |
| 204173_at | MYL6B | 217741_s_at | ZA20D2 |
| 222130_s_at | FTSJ2 | 211634_x_at | IGHM |
| 208583_x_at | HIST1H2AJ | 212150_at | KIAA0143 |
| 219464_at | CA14 | 202561_at | TNKS |
| 206667_s_at | SCAMP1 | 204079_at | TPST2 |
| 211697_x_at | LOC56902 | 215464_s_at | TAX1BP3 |
| 208675_s_at | DDOST | 208966_x_at | IFI16 |
| 220480_at | HAND2 | 215446_s_at | LOX |
| 203221_at | TLE1 | 211653_x_at | |
| 217968_at | TSSC1 | 211573_x_at | TGM2 |
| 217844_at | CTDSP1 | 201280_s_at | DAB2 |
| 203557_s_at | PCBD1 | 218418_s_at | ANKRD25 |
| 220107_s_at | C14orf140 | 218552_at | ECHDC2 |
| 210820_x_at | COQ7 | 212203_x_at | IFITM3 |
| 208478_s_at | BAX | 209699_x_at | AKR1C2 |
| 209805_at | PMS2 /// | 216269_s_at | ELN |
| | PMS2CL | | |
| 201791_s_at | DHCR7 | 204151_x_at | AKR1C1 |
| 206226_at | HRG | 203890_s_at | DAPK3 |
| 218873_at | GON4L | 202450_s_at | CTSK |
| 213272_s_at | LOC57146 | 211429_s_at | SERPI1 |
| 209302_at | POLR2H | 211991_s_at | HLA-DPA1 |
| 208676_s_at | PA2G4 | 201506_at | TGFBI |
| 215198_s_at | CALD1 | 219370_at | RPRM |
| 218636_s_at | MAN1B1 | 205471_s_at | DACH1 |
| 210589_s_at | GBA /// GBAP | 206332_s_at | IFI16 |
| 209516_at | SMYD5 | 202084_s_at | SEC14L1 |
| 218001_at | MRPS2 | 212937_s_at | COL6A1 |
| 216813_at | — | 202177_at | GAS6 |
| 209059_s_at | EDF1 | 209034_at | PNRC1 |
| 201405_s_at | COPS6 | 201371_s_at | CUL3 |
| 214061_at | WDR67 | 209083_at | CORO1A |
| 209701_at | ARTS-1 | 208146_s_at | CPVL |
| 213336_at | GTF2I | 213249_at | FBXL7 |
| 203720_s_at | ERCC1 | 202827_s_at | MMP14 |
| 208312_s_at | PRAMEF1 /// | 220595_at | PDZRN4 |
| | PRAMEF2 | | |
| 210501_x_at | EIF3S12 | 219179_at | DACT1 |
| 212487_at | KIAA0553 | 208091_s_at | ECOP |
| 204431_at | TLE2 | 209118_s_at | TUBA3 |
| 200708_at | GOT2 | 204298_s_at | LOX |
| 204676_at | C16orf51 | 217173_s_at | LDLR |
| 214546_s_at | P2RY11 | 210105_s_at | FYN |
| 203926_x_at | ATP5D | 204456_s_at | GAS1 |
| 214784_x_at | XPO6 | 222154_s_at | DPTP6 |
| 207501_s_at | FGF12 | 210269_s_at | RP13-297E16.1 |
| 203147_s_at | TRIM14 | 200033_at | DDX5 |
| 218168_s_at | CABC1 | 209168_at | GPM6B |
| 201904_s_at | CTDSPL | 206360_s_at | SOCS3 |
| 218548_x_at | TEX264 | 215116_s_at | DNM1 |
| 209247_s_at | ABCF2 | 203300_x_at | AP1S2 |
| 216315_x_at | UBE2V1 /// Kua- | 37408_at | MRC2 |
| | UEV | | |
| 215535_s_at | AGPAT1 | 209932_s_at | DUT |
| 220908_at | CCDC33 | 201278_at | DAB2 |
| 216525_x_at | PMS2L3 | 200784_s_at | LRP1 |
| 218464_s_at | C17orf63 | 213780_at | TCHH |
| 217872_at | NOP17 | 40359_at | RASSF7 |
| 203410_at | AP3M2 | 215411_s_at | TRAF3IP2 |
| 201511_at | AAMP | 216583_x_at | — |
| 210635_s_at | KLHL20 | 211536_x_at | MAP3K7 |
| 200895_s_at | FKBP4 | 201354_s_at | BAZ2A |
| 210113_s_at | LP1 | 204352_at | TRAF5 |
| 217961_at | FLJ20551 | 203854_at | CFI |
| 214473_x_at | PMS2L3 | 212938_at | COL6A1 |
| 213893_x_at | PMS2L5 /// | 204525_at | PHF14 |
| | LOC441259 /// | | |
| | LOC641799 /// | | |
| | LOC641800 /// | | |
| | LOC645243 /// | | |
| | LOC645248 | | |
| 217586_x_at | — | 222264_at | HNRPUL2 |
| 203364_s_at | KIAA0652 | 203567_s_at | TRIM38 |
| 217094_s_at | ITCH | 214366_s_at | ALOX5 |
| 218037_at | C2orf17 | 218290_at | PLEKHJ1 |
| 207511_s_at | C2orf24 | 215051_x_at | AIF1 |
| 219403_s_at | HPSE | 216028_at | DKFZP564C152 |
| 205795_at | NRXN3 | 208306_x_at | HLA-DRB1 |
| 214756_x_at | PMS2L1 | 202286_s_at | TACSTD2 |
| 218944_at | PYCRL | 213233_s_at | KLHL9 |
| 222006_at | LETM1 | 210026_s_at | CARD10 |
| 218004_at | BSDC1 | 209566_at | INSIG2 |
| 218673_s_at | ATG7 | 204907_s_at | BCL3 |
| 222176_at | PTEN | 217798_at | CNOT2 |
| 216843_x_at | PMS2L1 | 218864_at | TNS1 |
| 200851_s_at | KIAA0174 | 211065_x_at | PFKL |
| 221189_s_at | TARSL1 | 58780_s_at | FLJ10357 |
| 200990_at | TRIM28 | 221774_x_at | FAM48A |
| 221780_s_at | DDX27 | 209877_at | SNCG |
| 216267_s_at | TMEM115 | 211776_s_at | EPB41L3 |
| 220789_s_at | TBRG4 | 204150_at | STAB1 |
| 201905_s_at | CTDSPL | 208461_at | HIC1 |
| 209741_x_at | ZNF291 | 218454_at | FLJ22662 |
| 211127_x_at | EDA | 214250_at | NUMA1 |
| 218621_at | HEMK1 | 206743_s_at | ASGR1 |
| 202394_s_at | ABCF3 | 221901_at | KIAA1644 |
| 204476_s_at | PC | 209826_at | EGFL8 /// LOC653870 |
| 217209_at | — | 220318_at | EPN3 |
| 215321_at | RPIB9 | 204108_at | NFYA |
| 216514_at | — | 204882_at | ARHGAP25 |
| 214116_at | — | 218999_at | TMEM140 |
| 213957_s_at | CEP350 | 205135_s_at | NUFIP1 |
| 205610_at | MYOM1 | 217362_x_at | HLA-DRB6 |
| 214507_s_at | EXOSC2 | 209659_s_at | CDC16 |
| 217830_s_at | NSFL1C | 212552_at | HPCAL1 |
| 205851_at | NME6 | 219653_at | LSM14B |
| 217187_at | MUC5AC | 211001_at | TRIM29 |
| 202255_s_at | SIPA1L1 | 218614_at | C12orf35 |
| 205910_s_at | CEL | 209280_at | MRC2 |
| 204212_at | ACOT8 | 221934_s_at | DALRD3 |
| 214283_at | TMEM97 | 221447_s_at | GLT8D2 |
| 217485_x_at | PMS2L1 | 202099_s_at | DGCR2 |
| 206389_s_at | PDE3A | 209929_s_at | IKBKG |
| 221515_s_at | LCMT1 | 221483_s_at | ARPP-19 |
| 212712_at | CAMSAP1 | 203172_at | FXR2 |
| 207505_at | PRKG2 | 210245_at | ABCC8 |
| 221219_s_at | KLHDC4 | 205453_at | HOXB2 |
| 220444_at | ZNF557 | 201700_at | CCND3 |
| 207631_at | NBR2 | 204407_at | TTF2 |
| 210132_at | EF3 | 209777_s_at | SLC19A1 |
| 202570_s_at | DLGAP4 | 219729_at | PRRX2 |
| 202472_at | MPI | 206616_s_at | ADAM22 |
| 201377_at | UBAP2L | 211605_s_at | RARA |
| 203793_x_at | PCGF2 | 211208_s_at | CASK |
| 210022_at | PCGF1 | 213772_s_at | GGA2 |
| 206376_at | SLC6A15 | 202380_s_at | NKTR |
| 34868_at | SMG5 | 217125_at | — |
| 221049_s_at | POLL | 218182_s_at | CLDN1 |
| 217618_x_at | HUS1 | 221297_at | GPRC5D |
| 214199_at | SFTPD | 216928_at | TAL1 |
| 205631_at | KIAA0586 | 216017_s_at | B2 |
| 201966_at | NDUFS2 | 214084_x_at | LOC648998 /// |
| | | | LOC653361 /// |
| | | | LOC653840 |
| 222247_at | DXS542 | 210831_s_at | PTGER3 |
| 208420_x_at | SUPT6H | 216627_s_at | B4GALT1 |
| 211381_x_at | SPAG11 | 213443_at | TRADD |
| 219451_at | MSRB2 | 211322_s_at | SARDH |
| 218220_at | C12orf10 | 210344_at | OSBPL7 |
| 213952_s_at | ALOX5 | 220577_at | GVIN1 |
| 210695_s_at | WWOX | 211432_s_at | TYRO3 |
| 222120_at | MGC13138 | 221039_s_at | DDEF1 |
| 216568_x_at | — | 212869_x_at | TPT1 |
| 222184_at | — | 215242_at | PIGC |
| 218564_at | RFWD3 | 214327_x_at | TPT1 |
| 204883_s_at | HUS1 | 212284_x_at | TPT1 |
| 203918_at | PCDH1 | 211838_x_at | PCDHA5 |
| 215043_s_at | SMA3 /// SMA5 | 207676_at | ONECUT2 |
| 214070_s_at | ATP10B | 213888_s_at | TRAF3IP3 |
| 209165_at | AATF | 214390_s_at | BCAT1 |
| 221818_at | INTS5 | 221358_at | NPBWR2 |
| 222228_s_at | ALKBH4 | 205950_s_at | CA1 |
| 211977_at | GPR107 | 217136_at | PPIAL4 /// LOC653505 /// |
| | | | LOC653598 |
| 209743_s_at | ITCH | 221233_s_at | KIAA1411 |
| 222170_at | LOC440334 | 216839_at | LAMA2 |
| 204283_at | FARS2 | 215231_at | ABP1 |
| 216222_s_at | MYO10 | 216814_at | — |
| 212087_s_at | ERAL1 | 217321_x_at | ATXN3 |
| 213847_at | PRPH | 216819_at | — |
| 217538_at | RUTBC1 | 202865_at | DJB12 |
| 210192_at | ATP8A1 | 206490_at | DLGAP1 |
| 222064_s_at | AARSD1 | 207479_at | — |
| 219022_at | C12orf43 | 219688_at | BBS7 |
| 209423_s_at | PHF20 | 220791_x_at | SCN11A |
| 205699_at | — | 207465_at | — |
| 32402_s_at | SYMPK | AFFX- | — |
| | | PheX-5_at | |
| 220967_s_at | ZNF696 | 204884_s_at | HUS1 |
| 215931_s_at | ARFGEF2 | 217392_at | CAPZA1 |
| 202513_s_at | PPP2R5D | 214702_at | FN1 |
| 205666_at | FMO1 | 214636_at | CALCB |
| 212238_at | ASXL1 | 208181_at | HIST1H4H |
| 216091_s_at | BTRC | 215228_at | NHLH2 |
| 220086_at | ZNFN1A5 | 220507_s_at | UPB1 |
| 216204_at | COMT | 205539_at | AVIL |
| 210701_at | CFDP1 | 220869_at | UBE1L2 |
| 204717_s_at | SLC29A2 | 204945_at | PTPRN |
| 205334_at | S100A1 | 217048_at | — |
| 206941_x_at | SEMA3E | 215053_at | SRCAP |
| 212523_s_at | KIAA0146 | 221617_at | TAF9B |
| 206611_at | C2orf27 | 214222_at | DH7 |
| 219420_s_at | C1orf163 | 210520_at | FETUB |
| 214675_at | NUP188 | 220832_at | TLR8 |
| 217448_s_at | C14orf92 | 211310_at | EZH1 |
| 221440_s_at | RBBP9 | 221414_s_at | DEFB126 |
| 201763_s_at | DAXX | 206731_at | CNKSR2 |
| 216658_at | — | 215615_x_at | RERE |
| 212743_at | RCHY1 | 222048_at | ADRBK2 |
| 214842_s_at | ALB | 212743_at | RCHY1 |
| 204183_s_at | ADRBK2 | 213631_x_at | HP |
| 211566_x_at | BRE | 222176_at | PTEN |
| 204514_at | DPH2 | 213909_at | LRRC15 |
| 201184_s_at | CHD4 | 215611_at | TCF12 |
| 205355_at | ACADSB | 221409_at | OR2S2 |
| 217612_at | TIMM50 | 220793_at | SAGE1 |
| 215412_x_at | PMS2L2 | 206730_at | GRIA3 |
| 215430_at | GK2 | 217112_at | PDGFB |
| 200029_at | RPL19 | 215560_x_at | MTRF1L |
| 210712_at | LDHAL6B | 216422_at | PA2G4 |
| 204757_s_at | TMEM24 | 220776_at | KCNJ14 |
| 210197_at | ITPK1 | 206249_at | MAP3K13 |
| 220793_at | SAGE1 | 220764_at | PPP4R2 |
| 209802_at | PHLDA2 | 215768_at | SOX5 |
| 205115_s_at | RBM19 | 216536_at | OR7E19P |
| 214655_at | GPR6 | 207615_s_at | C16orf3 |
| 211402_x_at | NR6A1 | 203866_at | NLE1 |
| 219997_s_at | COPS7B | 205336_at | PVALB |
| 207044_at | THRB | 207254_at | SLC15A1 |
| 202707_at | UMPS | 203998_s_at | SYT1 |
| 220122_at | MCTP1 | 207236_at | ZNF345 |
| 205741_s_at | DT | 215652_at | |
| 221949_at | LOC222070 | 214675_at | NUP188 |
| 207772_s_at | PRMT8 | 210712_at | LDHAL6B |
| 202508_s_at | SP25 | 214655_at | GPR6 |
| 200045_at | ABCF1 | 221049_s_at | POLL |
| 207797_s_at | LRP2BP | 219997_s_at | COPS7B |
| 205322_s_at | MTF1 | 219928_s_at | CABYR |
| 202819_s_at | TCEB3 | 204191_at | IFR1 |
| 204652_s_at | NRF1 | 219711_at | ZNF586 |
| 203998_s_at | SYT1 | 215249_at | RPL35A |
| 221683_s_at | CEP290 | 215868_x_at | SOX5 |
| 219316_s_at | C14orf58 | 211402_x_at | NR6A1 |
| 220070_at | JMJD5 | 214245_at | RPS14 |
| 208145_at | LOC642671 | 207409_at | LECT2 |
| 207602_at | TMPRSS11D | 217612_at | TIMMS50 |
| 201684_s_at | C14orf92 | 207902_at | IL5RA |
| 206249_at | MAP3K13 | 210695_s_at | WWOX |
| 217454_at | LOC203510 | 216340_s_at | CYP2A7P1 |
| 220875_at | — | 217171_at | SMPD1 |
| 212092_at | PEG10 | 214842_s_at | ALB |
| 37278_at | TAZ | 221905_at | CYLD |
| 214901_at | ZNF8 | 205610_at | MYOM1 |
| 207459_x_at | GYPB | 210197_at | ITPK1 |
| 203866_at | NLE1 | 207045_at | FLJ20097 |
| 215834_x_at | SCARB1 | 210701_at | CFDP1 |
| 215768_at | SOX5 | 212308_at | CLASP2 |
| 213514_s_at | DIAPH1 | 201763_s_at | DAXX |
| 217238_s_at | ALDOB | 216661_x_at | CYP2C9 |
| 217071_s_at | MTHFR | 220122_at | MCTP1 |
| 216422_at | PA2G4 | 211318_s_at | RAE1 |
| 219198_at | GTF3C4 | 205915_x_at | GRIN1 |
| 210345_s_at | DH9 | 208281_x_at | DAZ1 /// DAZ3 /// DAZ2 |
| | | | /// DAZ4 |
| 210476_s_at | PRLR | 218564_at | RFWD3 |
| 206731_at | CNKSR2 | 213971_s_at | SUZ12 /// SUZ12P |
| 213732_at | TCF3 | 213957_s_at | CEP350 |
| 204945_at | PTPRN | 203839_s_at | TNK2 |
| 205521_at | ENDOGL1 | 214283_at | TMEM97 |
| 210520_at | FETUB | 217830_s_at | NSFL1C |
| 208537_at | EDG5 | 207331_at | CENPF |
| 213909_at | LRRC15 | 218621_at | HEMK1 |
| 208904_s_at | RPS28 /// | 207455_at | P2RY1 |
| | LOC645899 /// | | |
| | LOC646195 /// | | |
| | LOC651434 | | |
| 214557_at | PTTG2 | 220444_at | ZNF557 |
| 208140_s_at | LRRC48 | 201208_s_at | TNFAIP1 |
| 207254_at | SLC15A1 | 204283_at | FARS2 |
| 215656_at | LMAN2 | 202885_s_at | PPP2R1B |
| 219810_at | VCPIP1 | 203383_s_at | GOLGA1 |
| 207545_s_at | NUMB | 209072_at | MBP |
| 215228_at | NHLH2 | 203171_s_at | KIAA0409 |
| 216043_x_at | RAB11FIP3 | 202550_s_at | VAPB |
| 211310_at | EZH1 | 205851_at | NME6 |
| 219606_at | PHF20L1 | 217721_at | — |
| 215187_at | FLJ11292 | 210005_at | GART |
| 205539_at | AVIL | 207735_at | RNF125 |
| 216659_at | LOC647294 /// | 212087_s_at | ERAL1 |
| | LOC652593 | | |
| 221697_at | MAP1LC3C | 222184_at | — |
| 217048_at | — | 205238_at | CXorf34 |
| 216718_at | C1orf46 | 214526_x_at | PMS2L1 |
| 215433_at | DPY19L1 | 219543_at | MAWBP |
| 220564_at | C10orf59 | 204883_s_at | HUS1 |
| 217392_at | CAPZA1 | 217094_s_at | ITCH |
| 207465_at | — | 214756_x_at | PMS2L1 |
| 207331_at | CENPF | 207511_s_at | C2orf24 |
| 215419_at | KIAA1086 | 219854_at | ZNF14 |
| 217401_at | — | 213893_x_at | PMS2L5 /// LOC441259 /// |
| | | | LOC641799 /// |
| | | | LOC641800 /// |
| | | | LOC645243 /// |
| | | | LOC645248 |
| 210316_at | FLT4 | 207505_at | PRKG2 |
| 220049_s_at | PDCD1LG2 | 203436_at | RPP30 |
| 205106_at | MTCP1 | 205829_at | HSD17B1 |
| 206490_at | DLGAP1 | 201905_s_at | CTDSPL |
| 204884_s_at | HUS1 | 214507_s_at | EXOSC2 |
| AFFX-PheX-5_at | — | 209677_at | PRKCI |
| 44040_at | FBXO41 | 208676_s_at | PA2G4 |
| 211306_s_at | FCAR | 207347_at | ERCC6 |
| 220791_x_at | SCN11A | 201961_s_at | RNF41 |
| 220031_at | ZA20D1 | 209029_at | COPS7A |
| 216819_at | — | 219797_at | MGAT4A |
| 215516_at | LAMB4 | 219596_at | THAP10 |
| 216839_at | LAMA2 | 221984_s_at | C2orf17 |
| 204267_x_at | PKMYT1 | 222006_at | LETM1 |
| 215468_at | LOC647070 | 222192_s_at | FLJ21820 |
| 217136_at | PPIAL4 /// | 202004_x_at | SDHC /// LOC642502 |
| | LOC653505 /// | | |
| | LOC653598 | | |
| 220037_s_at | XLKD1 | 217586_x_at | — |
| 206962_x_at | — | 218540_at | THTPA |
| 204111_at | HNMT | 215198_s_at | CALD1 |
| 214681_at | GK | 217931_at | TNRC5 |
| 213888_s_at | TRAF3IP3 | 202801_at | PRKACA |
| 212284_x_at | TPT1 | 202821_s_at | LPP |
| 203015_s_at | SSX2IP | 208157_at | SIM2 |
| 204551_s_at | AHSG | 218636_s_at | MAN1B1 |
| 214327_x_at | TPT1 | 202924_s_at | PLAGL2 |
| 220491_at | HAMP | 219222_at | RBKS |
| 210931_at | RNF6 | 213328_at | NEK1 |
| 219901_at | FGD6 | 214473_x_at | PMS2L3 |
| 207503_at | TCP10 | 210187_at | FKBP1A |
| 219634_at | CHST11 | 200786_at | PSMB7 |
| 212869_x_at | TPT1 | 209222_s_at | OSBPL2 |
| 201319_at | MRCL3 | 205355_at | ACADSB |
| 219616_at | FLJ21963 | 214481_at | HIST1H2AM |
| 208018_s_at | HCK | 214315_x_at | CALR |
| 213273_at | ODZ4 | 221838_at | KLHL22 |
| 214543_x_at | QKI | 216315_x_at | UBE2V1 /// Kua-UEV |
| 213443_at | TRADD | 205047_s_at | ASNS |
| 208929_x_at | RPL13 | 218026_at | CCDC56 |
| 221356_x_at | P2RX2 | 204173_at | MYL6B |
| 209929_s_at | IKBKG | 211127_x_at | EDA |
| 220673_s_at | KIAA1622 | 207831_x_at | DHPS |
| 214649_s_at | MTMR2 | 218711_s_at | SDPR |
| 206715_at | TFEC | 203190_at | NDUFS8 |
| 201025_at | EIF5B | 202406_s_at | TIAL1 |
| 217687_at | ADCY2 | 52651_at | COL8A2 |
| 221447_s_at | GLT8D2 | 212684_at | ZNF3 |
| 209826_at | EGFL8 /// | 201791_s_at | DHCR7 |
| | LOC653870 | | |
| 212961_x_at | CXorf40B | 206667_s_at | SCAMP1 |
| 206801_at | NPPB | 214117_s_at | BTD |
| 218182_s_at | CLDN1 | 203368_at | CRELD1 |
| 219594_at | NINJ2 | 218658_s_at | ACTR8 |
| 203652_at | MAP3K11 | 219278_at | MAP3K6 |
| 221907_at | C14orf172 | 207156_at | HIST1H2AG |
| 213688_at | CALM1 | 214460_at | LSAMP |
| 204989_s_at | ITGB4 | 65884_at | MAN1B1 |
| 202055_at | KP1 | 221058_s_at | CKLF |
| 217362_x_at | HLA-DRB6 | 202903_at | LSM5 |
| 219055_at | SRBD1 | 201685_s_at | C14orf92 |
| 206987_x_at | FGF18 | 209231_s_at | DCTN5 |
| 201309_x_at | C5orf13 | 212862_at | CDS2 |
| 203017_s_at | SSX2IP | 219736_at | TRIM36 |
| 203227_s_at | TSPAN31 | 212283_at | AGRN |
| 207616_s_at | TANK | 202186_x_at | PPP2R5A |
| 221901_at | KIAA1644 | 209527_at | EXOSC2 |
| 202302_s_at | FLJ11021 | 200868_s_at | ZNF313 |
| 210933_s_at | FSCN1 | 209247_s_at | ABCF2 |
| 222148_s_at | RHOT1 | 204089_x_at | MAP3K4 |
| 213095_x_at | AIF1 | 214695_at | UBAP2L |
| 212613_at | BTN3A2 | 215203_at | GOLGA4 |
| 218013_x_at | DCTN4 | 203189_s_at | NDUFS8 |
| 210831_s_at | PTGER3 | 218830_at | RPL26L1 |
| 211776_s_at | EPB41L3 | 221860_at | HNRPL |
| 212535_at | MEF2A | 208523_x_at | HIST1H2BI |
| 201594_s_at | PPP4R1 | 218996_at | TFPT |
| 58780_s_at | FLJ10357 | 203593_at | CD2AP |
| 209658_at | CDC16 | 219125_s_at | RAG1AP1 |
| 202000_at | NDUFA6 | 218403_at | TRIAP1 |
| 205479_s_at | PLAU | 208490_x_at | HIST1H2BF |
| 211323_s_at | ITPR1 | 221261_x_at | MAGED4 /// LOC653210 |
| 210473_s_at | GPR125 | 208527_x_at | HIST1H2BE |
| 215051_x_at | AIF1 | 205501_at | — |
| 219078_at | GPATC2 | 209078_s_at | TXN2 |
| 212371_at | C1orf121 | 206110_at | HIST1H3H |
| 200978_at | MDH1 | 202098_s_at | PRMT2 |
| 202286_s_at | TACSTD2 | 208546_x_at | HIST1H2BH |
| 203705_s_at | FZD7 | 208579_x_at | H2BFS |
| 216583_x_at | — | 219538_at | WDR5B |
| 210102_at | LOH11CR2A | 212744_at | BBS4 |
| 203177_x_at | TFAM | 214472_at | HIST1H3D |
| 218534_s_at | AGGF1 | 215779_s_at | HIST1H2BG |
| 204215_at | C7orf23 | 208180_s_at | HIST1H4H |
| 218454_at | FLJ22662 | 214469_at | HIST1H2AE |
| 202794_at | INPP1 | 211474_s_at | SERPINB6 |
| 204037_at | EDG2 /// | 208583_x_at | HIST1H2AJ |
| | LOC644923 | | |
| 213233_s_at | KLHL9 | 215978_x_at | LOC152719 |
| 212222_at | PSME4 | 217775_s_at | RDH11 |
| 204222_s_at | GLIPR1 | 213789_at | — |
| 204456_s_at | GAS1 | 214455_at | HIST1H2BC |
| 211945_s_at | ITGB1 | 209210_s_at | PLEKHC1 |
| 217798_at | CNOT2 | | |
| 203567_s_at | TRIM38 | | |
| 203854_at | CFI | | |
| 200982_s_at | ANXA6 | | |
| 216231_s_at | B2M | | |
| 209901_x_at | AIF1 | | |
| 209083_at | CORO1A | | |
| 215116_s_at | DNM1 | | |
| 215411_s_at | TRAF3IP2 | | |
| 212314_at | KIAA0746 | | |
| 218047_at | OSBPL9 | | |
| 210273_at | PCDH7 | | |
| 217732_s_at | ITM2B | | |
| 208070_s_at | REV3L | | |
| 204150_at | STAB1 | | |
| 208985_s_at | EIF3S1 | | |
| 201278_at | DAB2 | | |
| 209550_at | NDN | | |
| 213741_s_at | KP1 | | |
| 210285_x_at | WTAP | | |
| 201887_at | IL13RA1 | | |
| 206117_at | TPM1 | | |
| 213716_s_at | SECTM1 | | |
| 202693_s_at | STK17A | | |
| 212500_at | C10orf22 | | |
| 219179_at | DACT1 | | |
| 219140_s_at | RBP4 | | |
| 203868_s_at | VCAM1 | | |
| 212294_at | GNG12 | | |
| 204298_s_at | LOX | | |
| 215313_x_at | HLA-A | | |
| 205698_s_at | MAP2K6 | | |
| 220955_x_at | RAB23 | | |
| 203300_x_at | AP1S2 | | |
| 209191_at | TUBB6 | | |
| 210915_x_at | TRBV19 /// | | |
| | TRBC1 | | |
| 200033_at | DDX5 | | |
| 202810_at | DRG1 | | |
| 218396_at | VPS13C | | |
| 204114_at | NID2 | | |
| 204364_s_at | REEP1 | | |
| 219687_at | HHAT | | |
| 201590_x_at | ANXA2 | | |
| 209168_at | GPM6B | | |
| 201060_x_at | STOM | | |
| 212203_x_at | IFITM3 | | |
| 213258_at | TFPI | | |
| 202450_s_at | CTSK | | |
| 204244_s_at | DBF4 | | |
| 210416_s_at | CHEK2 | | |
| 209932_s_at | DUT | | |
| 208146_s_at | CPVL | | |
| 203153_at | IFIT1 | | |
| 214252_s_at | CLN5 | | |
| 203961_at | NEBL | | |
| 204168_at | MGST2 | | |
| 40489_at | ATN1 | | |
| 209034_at | PNRC1 | | |
| 201280_s_at | DAB2 | | |
| 213572_s_at | SERPINB1 | | |
| 212586_at | CAST | | |
| 203323_at | CAV2 | | |
| 221816_s_at | PHF11 | | |
| 219370_at | RPRM | | |
| 201506_at | TGFBI | | |
| 201540_at | FHL1 | | |
| 211429_s_at | SERPI1 | | |
| 218656_s_at | LHFP | | |
| 210275_s_at | ZA20D2 | | |
| 201842_s_at | EFEMP1 | | |
| 201061_s_at | STOM | | |
| 209648_x_at | SOCS5 | | |
| 222088_s_at | SLC2A3 | | |
| 203706_s_at | FZD7 | | |
| 201132_at | HNRPH2 | | |
| 210139_s_at | PMP22 | | |
| 212149_at | KIAA0143 | | |
| 214257_s_at | SEC22B | | |
| 214022_s_at | IFITM1 | | |
| 218741_at | C22orf18 | | |
| 221523_s_at | RRAGD | | |
| 220595_at | PDZRN4 | | |
| 201601_x_at | IFITM1 | | |
| 202446_s_at | PLSCR1 | | |
| 206662_at | GLRX | | |
| 201560_at | CLIC4 | | |
| 206332_s_at | IFI16 | | |
| 217741_s_at | ZA20D2 | | |
| 202609_at | EPS8 | | |
| 202936_s_at | SOX9 | | |
| 209154_at | TAX1BP3 | | |
| 203305_at | F13A1 | | |
| 212824_at | FUBP3 | | |
| 208296_x_at | TNFAIP8 | | |
| 209498_at | CEACAM1 | | |
| 217832_at | SYNCRIP | | |
| 212533_at | WEE1 | | |
| 213193_x_at | TRBV19 /// | | |
| | TRBC1 | | |
| 204472_at | GEM | | |
| 205898_at | CX3CR1 | | |
| 200887_s_at | STAT1 | | |
| 209170_s_at | GPM6B | | |
| 209488_s_at | RBPMS | | |
| 210986_s_at | TPM1 | | |
| 204036_at | EDG2 | | |
| 208966_x_at | IFI16 | | |
| 202283_at | SERPINF1 | | |
| 203640_at | MBNL2 | | |
| 203810_at | DJB4 | | |
| 210072_at | CCL19 | | |
| 213791_at | PENK | | |
| 212230_at | PPAP2B | | |
| 210987_x_at | TPM1 | | |
| 205110_s_at | FGF13 | | |
| 212097_at | CAV1 | | |
| 215716_s_at | ATP2B1 | | |
| 200935_at | CALR | | |
| 218162_at | OLFML3 | | |
| 201645_at | TNC | | |
| 203710_at | ITPR1 | | |
| 211864_s_at | FER1L3 | | |
| 204939_s_at | PLN | | |
| 202430_s_at | PLSCR1 | | |
| 209487_at | RBPMS | | |
| 202037_s_at | SFRP1 | | |
| 204135_at | DOC1 | | |
| 206991_s_at | CCR5 /// | | |
| | LOC653725 | | |
| 200836_s_at | MAP4 | | |
| 209167_at | GPM6B | | |
| 212417_at | SCAMP1 | | |
| 210299_s_at | FHL1 | | |
| 209288_s_at | CDC42EP3 | | |
| 212671_s_at | HLA-DQA1 /// | | |
| | HLA-DQA2 /// | | |
| | LOC650946 | | |
| 209684_at | RIN2 | | |
| 201310_s_at | C5orf13 | | |
| 201196_s_at | AMD1 | | |
| 202269_x_at | GBP1 | | |
| 201798_s_at | FER1L3 | | |
| 204955_at | SRPX | | |
| 201787_at | FBLN1 | | |
| 209687_at | CXCL12 | | |
| 202291_s_at | MGP | | |
| 219117_s_at | FKBP11 | | |
| 207826_s_at | ID3 | | |
| 218730_s_at | OGN | | |
| 209291_at | ID4 | | |
| 209541_at | IGF1 | | |
| 204464_s_at | EDNRA | | |
| 201030_x_at | LDHB | | |
| 204172_at | CPOX | | |
| 217546_at | MT1M | | |
| 203453_at | SCNN1A | | |
| 203932_at | HLA-DMB | | |
| 205498_at | GHR | | |
| 213293_s_at | TRIM22 | | |
| 218087_s_at | SORBS1 | | |
| 205158_at | RSE4 | | |
| 216598_s_at | CCL2 | | |
| 213975_s_at | LYZ /// LILRB1 | | |
| 221510_s_at | GLS | | |
| 202258_s_at | PFAAP5 | | |
| 205097_at | SLC26A2 | | |
| 202333_s_at | UBE2B | | |
| 218589_at | P2RY5 | | |
| 202935_s_at | SOX9 | | |
| 213564_x_at | LDHB | | |
| 214836_x_at | IGKC /// IGKV1-5 | | |
| 204070_at | RARRES3 | | |
| 206392_s_at | RARRES1 | | |
| 218331_s_at | C10orf18 | | |
| 204259_at | MMP7 | | |
| 217028_at | CXCR4 | | |
| 221872_at | RARRES1 | | |
| 201650_at | KRT19 | | |

TABLE 9
Summary of Use of Independent Prostate Case Sets for
Gene Validation

| | p | up- | down- |
|---|---|---|---|
| Validation | threshlod | regulated | regulated |
| Significant Tumor Specific | | | |
| Relapse-associated Genes | | | |
| (Data set 1 & 3) | | | |
| data set 1 | p < 0.005 | 332 | 258 |
| data set 3 | p < 0.01 | 310 | 147 |
| Number of genes presented in | 22283 | | |
| both data set | | | |
| Number of overlapping significant | 15 | | |
| genes | | | |
| Number of overlapping significant | 12 | | |
| genes agreed in sign | | | |
| p value | 0.007 | | |
| Significant Stroma Specific | | | |
| Relapse-associated Genes | | | |
| (Data set 1 & 3) | | | |
| data set 1 | p < 0.005 | 197 | 219 |
| data set 3 | p < 0.01 | 200 | 474 |
| Number of genes presented in | 22283 | | |
| both data set | | | |
| Number of overlapping significant | 16 | | |
| genes | | | |
| Number of overlapping significant | 16 | | |
| genes agreed in sign | | | |
| p value | <0.001 | | |
| Significant Tumor Specific | | | |
| Relapse-associated Genes | | | |
| (Data set 1 & 2) | | | |
| data set 1 | p < 0.005 | 10 | 20 |
| data set 2 | p < 0.2 | 108 | 142 |
| Number of genes presented in | 730 | | |
| both data set | | | |
| Number of overlapping significant | 13 | | |
| genes | | | |
| Number of overlapping significant | 10 | | |
| genes agreed in sign | | | |
| p value | 0.011 | | |

TABLE 10
Tumor specific relapse related genes, identified by both dataset 1 and
dataset 3 using linear model.

| | U133A ID | Gene Symbol |
|---|---|---|
| Genes up-regulated | 208180_s_at | HIST1H4H |
| in relapse samples | 210052_s_at | TPX2 |
| | 219464_at | CA14 |
| | 221189_s_at | TARSL1 |
| | 205699_at | — |
| | 215768_at | SOX5 |
| Genes down-regulated | 215411_s_at | TRAF3IP2 |
| in relapse samples | 218047_at | OSBPL9 |
| | 212230_at | PPAP2B |
| | 202037_s_at | SFRP1 |
| | 205498_at | GHR |
| | 218589_at | P2RY5 |

TABLE 11
Stroma specific relapse related genes, identified by both dataset 1 and
dataset 3 using linear model.

| | U133A ID | Gene Symbol |
|---|---|---|
| Genes up-regulated | 201496_x_at | MYH11 |
| in relapse samples | 201367_s_at | ZFP36L2 |
| | 201495_x_at | MYH11 |
| | 203851_at | IGFBP6 |
| | 218552_at | ECHDC2 |
| | 215116_s_at | DNM1 |
| | 215411_s_at | TRAF3IP2 |
| Genes down-regulated | 220791_x_at | SCN11A |
| in relapse samples | 217392_at | CAPZA1 |
| | 220869_at | UBE1L2 |
| | 215768_at | SOX5 |
| | 215652_at | |
| | 208281_x_at | DAZ1 /// DAZ3 /// |
| | | DAZ2 /// DAZ4 |
| | 204883_s_at | HUS1 |
| | 214481_at | HIST1H2AM |
| | 212862_at | CDS2 |

TABLE 12
Tumor specific relapse related genes, identified by both dataset 1 and
dataset 2 using linear model.

| | U133A ID | Gene Symbol |
|---|---|---|
| Genes down-regulated | 209541_at | IGF1 |
| in relapse samples | 212097_at | CAV1 |
| | 212230_at | PPAP2B |
| | 201061_s_at | STOM |
| | 203323_at | CAV2 |
| | 201060_x_at | STOM |
| | 201590_x_at | ANXA2 |
| | 204298_s_at | LOX |
| | 211945_s_at | ITGB1 |

### Example 3

### In Silico Estimates of Tissue Components in Cancer Tissue Based on Expression Profiling Data

This example relates to the use of linear models to predict the tissue component of prostate samples based on microarray data. This strategy can be used to estimate the proportion of tissue components in each case and thereby reduce the impact of tissue proportions as a major source of variability among samples. The prediction model was tested by 10-fold cross validation within each data set, and also by mutual prediction across independent data sets.

Prostate cancer microarray data sets: Four publicly available prostate cancer data sets (datasets 1 through 4) with pathologist-estimated tissue component information were included in this study (Table 13). For all data sets, four major tissue components (tumor cells, stroma cells, epithelial cells of BPH, and epithelial cells of dilated cystic glands) were determined from sections prepared immediately before and after the sections pooled for RNA preparation by pathologists. The tissue component distributions for the four data sets are shown in Table 13.

Four publicly available microarray data sets (datasets 5 through 8) also were collected. These included a total of 238 arrays that were generated from 219 tumor enriched and 19 non-tumor parts of prostate tissue, as shown in Table 14. Dataset 5 consists of two groups (37 recurrence and 42 non-recurrence) for a total of 79 cases. The samples used in these four datasets do not have associated details of tissue component information.

Selection of Genes for Model-Training: Subsets of genes were selected to train the prediction model using two strategies. In the first strategy, each gene was ranked by the correlation coefficient between its intensity values and the percentage of a given tissue component across all samples. In the second strategy, the genes were ranked by their F-statistic, a measure of their fit in the multiple linear regression model as described below. The two strategies produced very similar results.

Multiple Linear Regression Model: A multi-variate linear regression model was used for prediction of tissue components. This is based on the assumption that the observed gene expression intensity of a gene is the summation of the contributions from different types of cells:

$$g = {β}_{0} + {\sum }_{j = 1}^{C} {β}_{j} {p}_{j} + e , \qquad ( 1 )$$

where g is the expression value for a gene, pⱼ is the percentage of a given tissue component determined by the pathologists, and βⱼ is the expression coefficient associated with a given cell type. In this model, C is the number of tissue types under consideration. In the current study, only β's of two major tissue types, tumor and stroma, were estimated to minimize the noise caused by other minority cell types. The contribution of other cell types to the total intensity g is subsumed into β₀ and e. Note that βⱼ is suggestive of the relative expression level in cell type j compared to the overall mean expression level β₀. The regression model was used to predict the percentage of tissue components after the parameters were determined on a training data set.

Cross-validation within data sets: Ten-fold cross-validation was used to estimate the prediction error rates for each data set. Briefly, one tenth of the samples were randomly selected as the test set using a boot strapping strategy and the remaining nine tenths of the samples were used as training set. Prediction models are constructed using the training sets with a pre-defined number of genes selected with the strategy mentioned above. The prediction is then tested on the test set. The sample selection and prediction step are repeated 10 times using different test samples each time until all the samples are used as test samples only once. This whole procedure is repeated five times using different sets of 10% of the data in each iteration to generate reliable results.

Validation between data sets: Mutual predictions were performed among datasets 1, 2, 3 and 4 to assess the applicability of prediction models across different data sets. Because the microarray platforms differ among the four data sets, quantile normalization are applied to preprocess the microarray data (Bolstad et al. (2003) Bioinformatics 19:185-193) with one modification. Quantile normalization method was applied on the test data set with the entire training set as the reference. This change means that the training set that is used to build prediction models will not be re-calculated and the prediction models will likely stay the same.

The mapping of probe sets from different Affymetrix platforms is based on the array comparison files downloaded from the Affymetrix website (World Wide Web at affymetrix.com). Probe sets of Probes in Affymetrix U133A array are a sublist of those in Affymetrix U133Plus2.0 array, and the DNA sequences of the common probes of two platforms are identical, suggesting these two platforms are very similar. The Illumina DASL platform used in data set 4 only provided gene symbols as the probe annotation, which was used to map to Affymetrix platforms. The numbers of genes mapped among different platforms are shown in Table 15.

Prediction on data sets that do not have pathologist's estimates of tissue proportions: Datasets 5, 6, 7, and 8 do not have previous estimates of tissue composition (Table 14). Datasets 1, 5, and 6 were generated from Affymetrix U133A arrays. Thus, the prediction models constructed with data set 1 were used to predict tissue components of samples used in datasets 5 and 6. Likewise, datasets 2, 7, and 8 were generated with Affymetrix U133Plus2.0 arrays, so prediction models constructed with dataset 2 were used to predict tissue components of samples used in datasets 7 and 8. The modified quantile normalization method described above was used for preprocessing the test data sets.

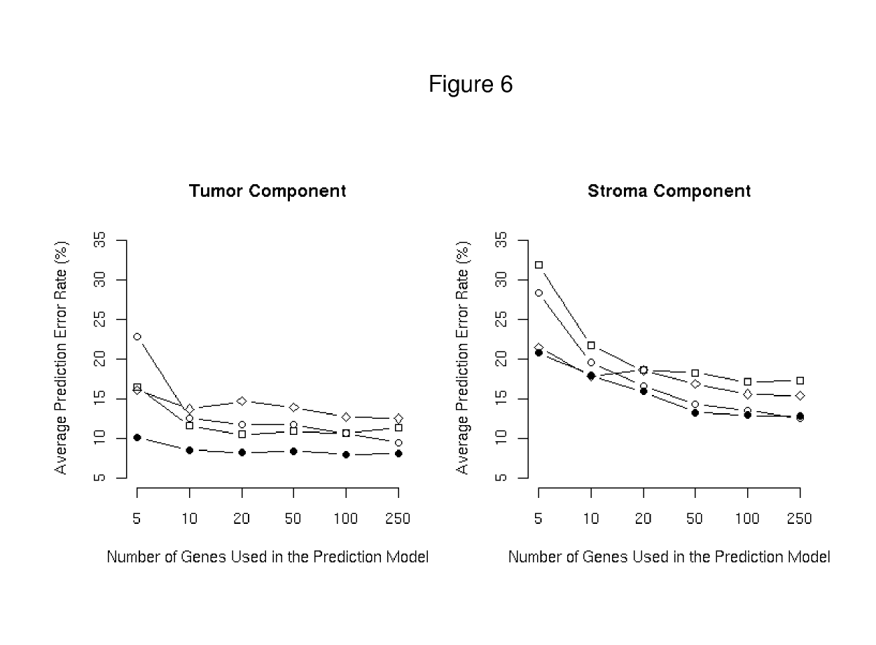

Comparison of in silico predictions and pathologist's estimates within the same data set: Four sets of microarray expression data for which tissue percentages had been determined by pathologists (Table 13), were used to develop in silico models that could predict tissue percentages in other samples that had array data but did not have pathologist data on tissue percentages. The discrepancies between in silico predictions and pathologist's estimates were measured by the mean absolute difference between values predicted in silico and the observation values estimated by pathologists. Ten-fold cross-validation was used to estimate the prediction discrepancies for datasets 1, 2, 3 and 4. To determine the best number of genes for constructing prediction model, the most significant 5, 10, 20, 50, 100 or 250 genes were compared. The prediction results are shown in FIGS. 6A and 6B, and Tables 16 and 17.

Among the four datasets, dataset 1 has the most similar in silico prediction to the pathologist's estimation, with 8% average discrepancy rate for tumor and 16% average discrepancy rate for stroma using the 250-gene model. This may because: 1) this dataset has four pathologists' estimation of tissue components, which will certainly be more accurate than that by one pathologist; 2) fresh frozen tissues were used which generate intact RNA for profiling; and/or 3) relatively larger sample size. Dataset 4 has the least accurate prediction, which may be because: 1) the dataset was generated from degraded total RNA samples from the FFPE blocks; and/or 2) the total number of genes on the Illumina DASL array platform are much less than that of other array platforms (511 probes versus 12626 or more probe sets for the other data sets).

The predictions of tumor components are slightly better than that of stroma, which may be explained in part by the fact that prostate stroma is a mixture of fibroblast cells, smooth muscle cells, blood vessels et al.

As shown in FIG. 6, the prediction model does not require many genes. The prediction model can reliable predict tumor components with as few as 10 genes, and predict stroma components with 50 genes.

Dataset 2 contains twelve laser capture micro-dissected tumor samples, the average in silico predicted tumor components for these samples are 91% in average. Assuming these samples really are all nearly pure tumor then the error rate is 9% or less for these samples, which is close to the average error rates of all samples in dataset 2.

The possibility of predicting of two other prostate cell types—the epithelial cells of BPH and dilated cystic glands by extending the current multi-variate model—also were explored. It was found that in silico prediction on these two tissue components are much less accurate than tumor and stroma component, largely because their percentage values are usually small and the pathologists differed in their estimates of these tissues. The extended prediction model including these tissues also slightly lowers the prediction accuracy of tumor and stroma components.

In the original study for dataset 3, agreement analysis on the tissue components that were estimated by four pathologists were assessed as inter-observer Pearson correlation coefficients. The average coefficients for tumor and stroma were 0.92 and 0.77. This is better than the correlation coefficients between in silico prediction and pathologist's estimation for the same dataset, which is 0.72 for the tumor component and 0.57 for stroma component. However, pathologists reviewed the same sections and the tissue components of the adjacent but non-identical samples processed for array assay may differ.

One indication that the prediction model may be optimized to the limits of the data available is the fact that the discrepancy between in silico predicted tissue components and pathologist's estimate for the predictions made on the test sets is often barely 1% different from that of the predictions made on the training set. See the example of 250-gene model as below. Data on other models were very similar.

Data set 1 (training/test): tumor 7.6%/8.1%; stroma 11.7%/12.8%.

Data set 2 (training/test): tumor 8.4%/9.5%; stroma 11.5%/12.5%.

Data set 3 (training/test): tumor 10.3%/11.4%; stroma 15.2%/17.3%.

Data set 4 (training/test): tumor 11.9%/12.5%; stroma 14.7%/15.4%.

To construct the best prediction models from each data set, a 10-fold permutation strategy was adopted to select the most suitable genes to be used in the final prediction model. To construct a n (i.e., 5, 10, 20, 50, 100, 250) gene model for each data set, only nine tenths of randomly chosen samples were used in the multi-variate linear regression analysis for selecting the n most significant genes. This step was repeated nine more times until all the samples were used nine times, which also means that all samples were skipped once. All selected genes (n×10) were pooled and ranked by their incidence. The n genes with the most hits, which are listed in Table 18, were used to construct prediction models that are integrated into CellPred program, as described below.

Comparison between in silico predictions across data sets and pathologist's estimates: Discrepancies for predictions made across different data sets are shown in Table 19. The 250-gene model is used for the mutual prediction. The prediction models constructed on fewer genes also were performed, and the prediction was less accurate than the 250-gene model. In general, the in silico predictions across different datasets are less similar to the pathologist's estimates than the in silico prediction made within the same dataset. However, the discrepancy in predictions across datasets is similar to the discrepancy within datasets when the array platforms are very similar (Affymetrix U133A and U133Plus2.0) and sample types are the same (i.e., fresh frozen sample). For the example of datasets 1 and 2, the prediction discrepancy is 11.0% for tumor and 16.7% for stroma when data set 1 was used as a training set, whereas vice versa, the numbers are 11.6% for tumor and 11.8% for stroma. In the case that microarray platforms and sample types vary (between fresh frozen and FFPE, for example), the cross data set prediction error rates increase and vary largely from 12.1% 28.6% for tumor and 14.7% to 38.2% for stroma depending on the comparison. The mutual prediction results strongly suggest that the feasibility of tissue components prediction across data sets when array platform and sample type are the same. For other cases, prediction of tissue percentages is also possible, but has a large error.

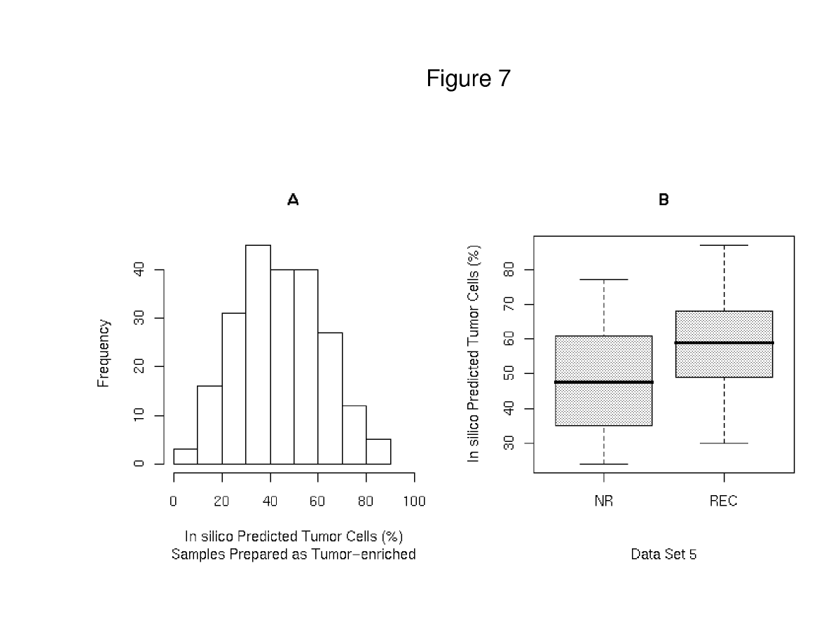

In silico prediction of tissue components of samples in publicly available prostate data sets: The in silico predicted tumor and stroma components of 238 samples used in datasets 5, 6, 7, and 8 are documented in Table 17. When 219 of 238 samples were prepared as tumor-enriched prostate tissue, the in silico predicted tumor proportions for these 219 samples showed a wide range from 0 to 87% tumor cells. There are 44 (20.1%) samples predicted with less than 30% tumor cells, as shown in FIG. 7A. These 44 samples with low amounts of predicted tumor appeared in dataset 5 (5 out of 79 tumor samples, 6.3%), dataset 6 (7 out of 44 tumor samples, 15.9%), dataset 7 (2 out of 13 tumor samples, 15.4%), and dataset 8 (30 out of 83 tumor samples, 36.1%), suggesting a large variation of tumor enrichment occurred in all the different data sets.

Dataset 5 includes information regarding recurrence of cancer after prostatectomy for patients, which was used to divide the samples into two groups for comparison (Stephenson, supra). The average tumor tissue component predicted for the recurrence group (58.5%) was noted to be about 10% higher than that of non-recurrence group (48.0%), as shown in FIG. 7B. Unless recognized and taken into account, this skew has the potential to provide false data regarding recurrence. Thus, tumor-specific genes are enriched in univariate analysis of the recurrent cases simply because such genes are naturally enriched in samples with more tumor cells.

To further illustrate this effect, the percentage of tumor predicted on dataset 5 using the dataset 1 in silico model was plotted as the x axis in a heat map with the non-recurrence and recurrence groups plotted separately. The Y axis consists of the expression levels in data set 5 of the top 100 (50 up- and 50 down-regulated) significant differential expressed genes between tumor and normal tissue identified in dataset 6. The gradient effects from left to right on two groups (non-recurrence and recurrence group) of samples from dataset 5 shows that expression levels of tissue specific genes selected from dataset 6 greatly correlate with the in silico predicted tumor contents with the prediction models developed from dataset 1. Moreover, samples in the recurrence group show slightly higher expression levels in up-regulated genes and lower expression level in down-regulated genes (also shown in FIG. 7B), indicating that the tumor components vary among two groups that may cause bias if two groups were compared directly without corrections.

Software for prostate cancer tissue prediction: CellPred, a web service freely available on the World Wide Web at webarraydb.org, was designed for prediction of the tissue components of prostate samples used in high-throughput expression studies, such as microarrays. CellPred was developed on a LAMP system (a GNU Linux server with Apache, MySQL and Python). The modules were written in python (World Wide Web at python.org) while analysis functions were written in R language (World Wide Web at r-project.org). The R script for modeling/training/prediction is downloadable from the World Wide Web at webarraydb.org/softwares/CellPred/. Users have the option to choose the number of genes for constructing the model. Genes used for generating the model are provided as an output file. Other details about the program can be found in the online help document.

Users can upload their own data sets for construction of prediction models. However, as an example, data has already been uploaded to allow prediction models constructed on datasets 1, 2 and 3 to be used for making predictions for a user-supplied data set. The user needs to upload the Affymetrix Cel file or any other type of microarray intensity file processed appropriately to make it compatible for making predictions. The most accurate prediction is made for Affymetrix U133A, U133Plus2.0 and U95Av2 array data using the prediction models developed on dataset 1, 2, or 3 respectively. For all other types of microarray platforms, prediction is likely quite noisy. In such cases, probes/probe sets on the platform of the test sets will be mapped to the probes on the training set of choice based on the gene symbols, gene IDs (i.e. GenBank IDs, refSeq IDs) or a mapping file (Xia et al. (2009) Bioinformatics 25:2425-2429). Modified quantile normalization is integrated for preprocessing the intensity values of the test arrays. Then the prediction is made on the test sets using the prediction models constructed with the training set. High-throughput expression sequence tags are accepted by the program if the data are condensed into a file equivalent to an intensity file, along with gene names or IDs that can be mapped to the training data sets.

TABLE 13
Prostate cancer microarray data sets with known tissue component information.

| | | Data Set 1 | Data Set 2 | Data Set 3 | Data Set 4 |
|---|---|---|---|---|---|
| Microarray Platform | | U133A | U133Plus2 | U95Av2 | Illumina DASL |
| | | | | | arrays |
| Sample Type | | Fresh | Fresh | Fresh Frozen | FFPE |
| | | Frozen | Frozen | | |
| n. of Arrays | | 136 | 149 | 88 | 114 |
| Sample Source | Prostatectomy | 132 | 110 | 88 | 114 |
| | Autopsy* | 4 | 13 | | |
| | LCM** | | 16\hat{ } | | |
| | Prostate | | 10 | | |
| | Biopsy | | | | |
| Data Source | | GSE8218 | GSE17951 | GSE1431*** | **** |
| n. of Probes or Probe | | | | | |
| Sets | | 22283 | 54675 | 12626 | 511 |
| n. of Pathologists | | 4 | 1 | 4 | 1 |
| Tumor (%) | Maximum | 80 | 100 | 80 | 90 |
| | Mean | 20 | 26 | 17 | 24 |
| | Minimum | 0 | 0 | 0 | 0 |
| Stroma (%) | Maximum | 100 | 100 | 100 | 100 |
| | Mean | 61 | 63 | 59 | 54 |
| | Minimum | 4 | 0 | 4 | 0 |
| Epithelium from BPH | Maximum | 50 | 53 | 55 | 60 |
| (%) | Mean | 11 | 6 | 12 | 14 |
| | Minimum | 0 | 0 | 0 | 0 |
| Atrophic Gland (%) | Maximum | 20 | 49 | 32 | 50 |
| | Mean | 6 | 4 | 7 | 7 |
| | Minimum | 0 | 0 | 0 | 0 |

*Autopsy prostate samples from normal subjects.
**Laser capture micro-dissected samples;
\hat{ }12 tumor samples and 4 stroma samples.
***Stuart et al., supra
**** Bibikova et al. (2007) Genomics 89: 666-672

TABLE 14
Prostate cancer microarray data sets without known tissue component information.

| | Data Set 5 | Data Set 6 | Data Set 7 | Data Set 8 |
|---|---|---|---|---|
| Array Platform | U133A | U133A | U133Plus2 | U133Plus2 |
| n. of Arrays | 79 | 57 | 19 | 83 |
| Sample Type | Fresh | Fresh Frozen | Fresh | Fresh |
| | Frozen | | Frozen | Frozen |
| Tumor-enriched | 79 | 44 | 13 | 83 |
| Samples | | | | |
| Stroma Samples | 0 | 13 | 6 | 0 |
| Data Source | * | http://www.ebi.ac.uk/microarray- | GSE3225 | GSE2109 |
| | | as/ae/browse.html?keywords=E- | | |
| | | TABM-26 | | |

TABLE 15
In silico tissue components (tumor/stroma) prediction discrepancies
(%) and correlation coefficients compared to pathologist's
estimates using 10-fold cross validation.

| | | Data Set 1 | Data Set 2 | Data Set 3 | Data Set 4 |
|---|---|---|---|---|---|
| 5-gene model | Tumor | 10.1/0.78 | 22.9/0.41 | 16.5/0.48 | 16.1/0.64 |
| | Cells | | | | |
| | Stroma | 20.8/0.51 | 28.4/0.38 | 31.9/0.16 | 21.5/0.5 |
| 10-gene model | Tumor | 8.5/0.83 | 12.6/0.84 | 11.6/0.7 | 13.7/0.71 |
| | Cells | | | | |
| | Stroma | 18/0.57 | 19.6/0.61 | 21.7/0.52 | 17.8/0.62 |
| 20-gene model | Tumor | 8.2/0.85 | 11.8/0.86 | 10.5/0.74 | 14.7/0.63 |
| | Cells | | | | |
| | Stroma | 15.9/0.64 | 16.6/0.72 | 18.6/0.5 | 18.6/0.6 |
| 50-gene model | Tumor | 8.4/0.86 | 11.7/0.85 | 10.9/0.72 | 13.9/0.69 |
| | Cells | | | | |
| | Stroma | 13.3/0.72 | 14.3/0.78 | 18.3/0.55 | 16.9/0.66 |
| 100-gene | Tumor | 8/0.87 | 10.6/0.87 | 10.6/0.75 | 12.7/0.7 |
| model | Cells | | | | |
| | Stroma | 12.9/0.74 | 13.5/0.79 | 17.1/0.56 | 15.6/0.7 |
| 250-gene | Tumor | 8.1/0.87 | 9.5/0.9 | 11.4/0.72 | 12.5/0.73 |
| model | Cells | | | | |
| | Stroma | 12.8/0.73 | 12.5/0.82 | 17.3/0.57 | 15.4/0.72 |

TABLE 16
Number of probes/probe sets mapped across different
microarray platforms.

| | | | | Illumina |
|---|---|---|---|---|
| | U133A | U133Plus2.0 | U95Av2 | DASL array |
| U133A | — | — | — | — |
| U133Plus2.0 | 22277 | — | — | — |
| U95Av2 | 12310 | 12323 | — | — |
| Illumina DASL array | 359 | 359 | 330 | — |

TABLE 17
In silico predicted tissue components for datasets 5, 6, 7 and 8 (%).

| Data Sets | sample name | sample type | Platform | Tumor | Stroma |
|---|---|---|---|---|---|
| Data Set 5 | SL_U133A_PG_12 | tumor-enriched samples | U133A | 75 | 25 |
| Data Set 5 | SL_U133A_PG_42 | tumor-enriched samples | U133A | 42 | 48 |
| Data Set 5 | SL_U133A_PG_45 | tumor-enriched samples | U133A | 42 | 58 |
| Data Set 5 | SL_U133A_PG_50 | tumor-enriched samples | U133A | 70 | 30 |
| Data Set 5 | SL_U133A_PG_53 | tumor-enriched samples | U133A | 31 | 69 |
| Data Set 5 | SL_U133A_PG_8 | tumor-enriched samples | U133A | 38 | 60 |
| Data Set 5 | SL_U133A_PR22.T | tumor-enriched samples | U133A | 61 | 29 |
| Data Set 5 | SL_U133A_PR24.T | tumor-enriched samples | U133A | 63 | 34 |
| Data Set 5 | SL_U133A_PR25.T | tumor-enriched samples | U133A | 61 | 31 |
| Data Set 5 | SL_U133A_PR28.T | tumor-enriched samples | U133A | 35 | 65 |
| Data Set 5 | SL_U133A_PR31.T | tumor-enriched samples | U133A | 52 | 47 |
| Data Set 5 | SL_U133A_PR32.T | tumor-enriched samples | U133A | 60 | 33 |
| Data Set 5 | SL_U133A_PR33.T | tumor-enriched samples | U133A | 39 | 46 |
| Data Set 5 | SL_U133A_PR35.T | tumor-enriched samples | U133A | 62 | 37 |
| Data Set 5 | SL_U133A_PR37.T | tumor-enriched samples | U133A | 77 | 23 |
| Data Set 5 | SL_U133A_PR39.T | tumor-enriched samples | U133A | 31 | 69 |
| Data Set 5 | SL_U133A_PR40.T | tumor-enriched samples | U133A | 47 | 52 |
| Data Set 5 | SL_U133A_PR41.T | tumor-enriched samples | U133A | 25 | 75 |
| Data Set 5 | SL_U133A_PR42.T | tumor-enriched samples | U133A | 61 | 32 |
| Data Set 5 | SL_U133A_PR43.T | tumor-enriched samples | U133A | 66 | 34 |
| Data Set 5 | SL_U133A_PR44.T | tumor-enriched samples | U133A | 35 | 53 |
| Data Set 5 | SL_U133A_PR45.T | tumor-enriched samples | U133A | 37 | 31 |
| Data Set 5 | SL_U133A_PR47.T | tumor-enriched samples | U133A | 66 | 34 |
| Data Set 5 | SL_U133A_PR50.T | tumor-enriched samples | U133A | 48 | 45 |
| Data Set 5 | SL_U133A_PR52.T | tumor-enriched samples | U133A | 69 | 30 |
| Data Set 5 | SL_U133A_PR53.T | tumor-enriched samples | U133A | 56 | 42 |
| Data Set 5 | SL_U133A_PR54.T | tumor-enriched samples | U133A | 65 | 35 |
| Data Set 5 | SL_U133A_PR55.T | tumor-enriched samples | U133A | 25 | 47 |
| Data Set 5 | SL_U133A_PR56.T | tumor-enriched samples | U133A | 51 | 31 |
| Data Set 5 | SL_U133A_PR57.T | tumor-enriched samples | U133A | 27 | 57 |
| Data Set 5 | SL_U133A_PR58.T | tumor-enriched samples | U133A | 33 | 42 |
| Data Set 5 | SL_U133A_PR59.T.REP | tumor-enriched samples | U133A | 32 | 68 |
| Data Set 5 | SL_U133A_PR60.T | tumor-enriched samples | U133A | 55 | 45 |
| Data Set 5 | SL_U133A_PR61.T | tumor-enriched samples | U133A | 60 | 35 |
| Data Set 5 | SL_U133A_PR62.T | tumor-enriched samples | U133A | 24 | 50 |
| Data Set 5 | SL_U133A_PR64.T | tumor-enriched samples | U133A | 45 | 55 |
| Data Set 5 | SL_U133A_PR65.T | tumor-enriched samples | U133A | 57 | 43 |
| Data Set 5 | SL_U133A_PR66.T | tumor-enriched samples | U133A | 53 | 47 |
| Data Set 5 | SL_U133A_PR68.T | tumor-enriched samples | U133A | 45 | 42 |
| Data Set 5 | SL_U133A_PR69.T | tumor-enriched samples | U133A | 33 | 56 |
| Data Set 5 | SL_U133A_PR70.T | tumor-enriched samples | U133A | 29 | 71 |
| Data Set 5 | SL_U133A_PR71.T | tumor-enriched samples | U133A | 35 | 48 |
| Data Set 5 | SL_U133A_PG_13 | tumor-enriched samples | U133A | 67 | 33 |
| Data Set 5 | SL_U133A_PG_15 | tumor-enriched samples | U133A | 33 | 64 |
| Data Set 5 | SL_U133A_PG_37 | tumor-enriched samples | U133A | 72 | 28 |
| Data Set 5 | SL_U133A_PG_41 | tumor-enriched samples | U133A | 59 | 35 |
| Data Set 5 | SL_U133A_PG_46 | tumor-enriched samples | U133A | 49 | 51 |
| Data Set 5 | SL_U133A_PG_52 | tumor-enriched samples | U133A | 64 | 36 |
| Data Set 5 | SL_U133A_PR10.T | tumor-enriched samples | U133A | 60 | 40 |
| Data Set 5 | SL_U133A_PR11.T | tumor-enriched samples | U133A | 35 | 61 |
| Data Set 5 | SL_U133A_PR12.Trpt | tumor-enriched samples | U133A | 46 | 54 |
| Data Set 5 | SL_U133A_PR13.T | tumor-enriched samples | U133A | 60 | 31 |
| Data Set 5 | SL_U133A_PR14.T | tumor-enriched samples | U133A | 41 | 46 |
| Data Set 5 | SL_U133A_PR15.T | tumor-enriched samples | U133A | 52 | 39 |
| Data Set 5 | SL_U133A_PR16.T | tumor-enriched samples | U133A | 87 | 13 |
| Data Set 5 | SL_U133A_PR17.T | tumor-enriched samples | U133A | 61 | 31 |
| Data Set 5 | SL_U133A_PR18.T | tumor-enriched samples | U133A | 73 | 27 |
| Data Set 5 | SL_U133A_PR19.T | tumor-enriched samples | U133A | 68 | 32 |
| Data Set 5 | SL_U133A_PR1.Tredo | tumor-enriched samples | U133A | 39 | 45 |
| Data Set 5 | SL_U133A_PR20.T | tumor-enriched samples | U133A | 57 | 43 |
| Data Set 5 | SL_U133A_PR21.Trep | tumor-enriched samples | U133A | 62 | 38 |
| Data Set 5 | SL_U133A_PR26.T | tumor-enriched samples | U133A | 34 | 66 |
| Data Set 5 | SL_U133A_PR27.T | tumor-enriched samples | U133A | 42 | 51 |
| Data Set 5 | SL_U133A_PR29.T | tumor-enriched samples | U133A | 82 | 18 |
| Data Set 5 | SL_U133A_PR2.Tredo | tumor-enriched samples | U133A | 50 | 50 |
| Data Set 5 | SL_U133A_PR3.TREDO | tumor-enriched samples | U133A | 59 | 41 |
| Data Set 5 | SL_U133A_PR48.T | tumor-enriched samples | U133A | 74 | 26 |
| Data Set 5 | SL_U133A_PR49.T | tumor-enriched samples | U133A | 53 | 38 |
| Data Set 5 | SL_U133A_PR4.TREDO | tumor-enriched samples | U133A | 30 | 60 |
| Data Set 5 | SL_U133A_PR51.T | tumor-enriched samples | U133A | 58 | 30 |
| Data Set 5 | SL_U133A_PR5.TREDO | tumor-enriched samples | U133A | 82 | 18 |
| Data Set 5 | SL_U133A_PR63.T | tumor-enriched samples | U133A | 48 | 51 |
| Data Set 5 | SL_U133A_PR6.TREDO | tumor-enriched samples | U133A | 61 | 39 |
| Data Set 5 | SL_U133A_PR72.T | tumor-enriched samples | U133A | 72 | 28 |
| Data Set 5 | SL_U133A_PR73.T | tumor-enriched samples | U133A | 68 | 21 |
| Data Set 5 | SL_U133A_PR74.B | tumor-enriched samples | U133A | 84 | 16 |
| Data Set 5 | SL_U133A_PR7.TRED02 | tumor-enriched samples | U133A | 49 | 32 |
| Data Set 5 | SL_U133A_PR8.TREDO | tumor-enriched samples | U133A | 76 | 24 |
| Data Set 5 | SL_U133A_PR9.TREDO | tumor-enriched samples | U133A | 56 | 44 |
| Data Set 6 | A-1940339465.CEL | tumor-enriched samples | U133A | 37 | 33 |
| Data Set 6 | A-2393346053.CEL | tumor-enriched samples | U133A | 62 | 30 |
| Data Set 6 | A-3010184133.CEL | tumor-enriched samples | U133A | 67 | 28 |
| Data Set 6 | A-3435720971.CEL | tumor-enriched samples | U133A | 59 | 35 |
| Data Set 6 | A-4418592762.CEL | tumor-enriched samples | U133A | 62 | 30 |
| Data Set 6 | A-4464625690.CEL | tumor-enriched samples | U133A | 12 | 34 |
| Data Set 6 | A-4472570235.CEL | tumor-enriched samples | U133A | 61 | 36 |
| Data Set 6 | A-4917290232.CEL | tumor-enriched samples | U133A | 74 | 19 |
| Data Set 6 | A-4963842013.CEL | tumor-enriched samples | U133A | 18 | 63 |
| Data Set 6 | A-5173529673.CEL | tumor-enriched samples | U133A | 62 | 38 |
| Data Set 6 | A-5292628126.CEL | tumor-enriched samples | U133A | 37 | 39 |
| Data Set 6 | A-5642567629.CEL | tumor-enriched samples | U133A | 80 | 18 |
| Data Set 6 | A-7270793196.CEL | tumor-enriched samples | U133A | 0 | 84 |
| Data Set 6 | A-7350218006.CEL | tumor-enriched samples | U133A | 20 | 53 |
| Data Set 6 | A-8500920543.CEL | tumor-enriched samples | U133A | 44 | 45 |
| Data Set 6 | A-9763059872.CEL | tumor-enriched samples | U133A | 43 | 36 |
| Data Set 6 | 111T-A.CEL | tumor-enriched samples | U133A | 44 | 43 |
| Data Set 6 | A-135T.CEL | tumor-enriched samples | U133A | 38 | 39 |
| Data Set 6 | A-169T.CEL | tumor-enriched samples | U133A | 45 | 49 |
| Data Set 6 | A-171T.CEL | tumor-enriched samples | U133A | 62 | 38 |
| Data Set 6 | A-185N.CEL | stroma samples | U133A | 0 | 69 |
| Data Set 6 | 185T-A.CEL | tumor-enriched samples | U133A | 49 | 31 |
| Data Set 6 | 195T-A.CEL | tumor-enriched samples | U133A | 46 | 42 |
| Data Set 6 | A-226T.CEL | tumor-enriched samples | U133A | 43 | 46 |
| Data Set 6 | A-237T.CEL | tumor-enriched samples | U133A | 37 | 57 |
| Data Set 6 | A-23N.CEL | stroma samples | U133A | 19 | 78 |
| Data Set 6 | A-23T.CEL | tumor-enriched samples | U133A | 48 | 52 |
| Data Set 6 | 243T-A.CEL | tumor-enriched samples | U133A | 53 | 38 |
| Data Set 6 | 246T-A.CEL | tumor-enriched samples | U133A | 45 | 55 |
| Data Set 6 | A-257T.CEL | tumor-enriched samples | U133A | 58 | 39 |
| Data Set 6 | A-340N.CEL | stroma samples | U133A | 25 | 52 |
| Data Set 6 | 340T.CEL | tumor-enriched samples | U133A | 32 | 68 |
| Data Set 6 | 357T.CEL | tumor-enriched samples | U133A | 51 | 49 |
| Data Set 6 | 362T.CEL | tumor-enriched samples | U133A | 46 | 54 |
| Data Set 6 | 370T.CEL | tumor-enriched samples | U133A | 36 | 50 |
| Data Set 6 | A-399N.CEL | stroma samples | U133A | 0 | 63 |
| Data Set 6 | 399T.CEL | tumor-enriched samples | U133A | 15 | 85 |
| Data Set 6 | 405T.CEL | tumor-enriched samples | U133A | 38 | 39 |
| Data Set 6 | A-EP01N.CEL | stroma samples | U133A | 0 | 77 |
| Data Set 6 | A-EP01T.CEL | tumor-enriched samples | U133A | 24 | 73 |
| Data Set 6 | A-EP02N.CEL | stroma samples | U133A | 5 | 71 |
| Data Set 6 | A-EP02T.CEL | tumor-enriched samples | U133A | 38 | 62 |
| Data Set 6 | A-EP03N.CEL | stroma samples | U133A | 8 | 56 |
| Data Set 6 | A-EP03T.CEL | tumor-enriched samples | U133A | 41 | 53 |
| Data Set 6 | A-EP04N.CEL | stroma samples | U133A | 0 | 65 |
| Data Set 6 | A-EP04T.CEL | tumor-enriched samples | U133A | 30 | 53 |
| Data Set 6 | A-EP06N.CEL | stroma samples | U133A | 0 | 76 |
| Data Set 6 | A-EP06T.CEL | tumor-enriched samples | U133A | 38 | 61 |
| Data Set 6 | A-V16N.CEL | stroma samples | U133A | 7 | 69 |
| Data Set 6 | A-V16T2.CEL | tumor-enriched samples | U133A | 13 | 73 |
| Data Set 6 | A-V19N.CEL | stroma samples | U133A | 0 | 67 |
| Data Set 6 | A-V19T.CEL | tumor-enriched samples | U133A | 32 | 56 |
| Data Set 6 | A-V21N.CEL | stroma samples | U133A | 10 | 82 |
| Data Set 6 | A-V21T.CEL | tumor-enriched samples | U133A | 58 | 42 |
| Data Set 6 | A-V29N.CEL | stroma samples | U133A | 0 | 82 |
| Data Set 6 | A-V29T.CEL | tumor-enriched samples | U133A | 42 | 38 |
| Data Set 6 | A-V30T.CEL | tumor-enriched samples | U133A | 41 | 30 |
| Data Set 7 | GSM74875.CEL | stroma samples | U133P2 | 9 | 91 |
| Data Set 7 | GSM74876.CEL | stroma samples | U133P2 | 21 | 68 |
| Data Set 7 | GSM74877.CEL | stroma samples | U133P2 | 2 | 98 |
| Data Set 7 | GSM74878.CEL | stroma samples | U133P2 | 19 | 76 |
| Data Set 7 | GSM74879.CEL | stroma samples | U133P2 | 10 | 90 |
| Data Set 7 | GSM74880.CEL | stroma samples | U133P2 | 9 | 91 |
| Data Set 7 | GSM74881.CEL | tumor-enriched samples | U133P2 | 33 | 67 |
| Data Set 7 | GSM74882.CEL | tumor-enriched samples | U133P2 | 26 | 74 |
| Data Set 7 | GSM74883.CEL | tumor-enriched samples | U133P2 | 37 | 63 |
| Data Set 7 | GSM74884.CEL | tumor-enriched samples | U133P2 | 41 | 59 |
| Data Set 7 | GSM74885.CEL | tumor-enriched samples | U133P2 | 32 | 68 |
| Data Set 7 | GSM74886.CEL | tumor-enriched samples | U133P2 | 34 | 66 |
| Data Set 7 | GSM74887.CEL | tumor-enriched samples | U133P2 | 34 | 66 |
| Data Set 7 | GSM74888.CEL | tumor-enriched samples | U133P2 | 82 | 18 |
| Data Set 7 | GSM74889.CEL | tumor-enriched samples | U133P2 | 76 | 24 |
| Data Set 7 | GSM74890.CEL | tumor-enriched samples | U133P2 | 61 | 39 |
| Data Set 7 | GSM74891.CEL | tumor-enriched samples | U133P2 | 59 | 41 |
| Data Set 7 | GSM74892.CEL | tumor-enriched samples | U133P2 | 75 | 25 |
| Data Set 7 | GSM74893.CEL | tumor-enriched samples | U133P2 | 72 | 28 |
| Data Set 8 | GSM38079.CEL | tumor-enriched samples | U133P2 | 29 | 71 |
| Data Set 8 | GSM46837.CEL | tumor-enriched samples | U133P2 | 58 | 42 |
| Data Set 8 | GSM46866.CEL | tumor-enriched samples | U133P2 | 40 | 60 |
| Data Set 8 | GSM137971.CEL | tumor-enriched samples | U133P2 | 54 | 46 |
| Data Set 8 | GSM138038.CEL | tumor-enriched samples | U133P2 | 48 | 36 |
| Data Set 8 | GSM152575.CEL | tumor-enriched samples | U133P2 | 51 | 49 |
| Data Set 8 | GSM152611.CEL | tumor-enriched samples | U133P2 | 64 | 32 |
| Data Set 8 | GSM152617.CEL | tumor-enriched samples | U133P2 | 23 | 73 |
| Data Set 8 | GSM152622.CEL | tumor-enriched samples | U133P2 | 19 | 76 |
| Data Set 8 | GSM152631.CEL | tumor-enriched samples | U133P2 | 20 | 80 |
| Data Set 8 | GSM152772.CEL | tumor-enriched samples | U133P2 | 38 | 62 |
| Data Set 8 | GSM152778.CEL | tumor-enriched samples | U133P2 | 59 | 41 |
| Data Set 8 | GSM152783.CEL | tumor-enriched samples | U133P2 | 36 | 64 |
| Data Set 8 | GSM179790.CEL | tumor-enriched samples | U133P2 | 27 | 73 |
| Data Set 8 | GSM179792.CEL | tumor-enriched samples | U133P2 | 31 | 69 |
| Data Set 8 | GSM179843.CEL | tumor-enriched samples | U133P2 | 28 | 72 |
| Data Set 8 | GSM179849.CEL | tumor-enriched samples | U133P2 | 15 | 85 |
| Data Set 8 | GSM102498.CEL | tumor-enriched samples | U133P2 | 46 | 54 |
| Data Set 8 | GSM102510.CEL | tumor-enriched samples | U133P2 | 35 | 65 |
| Data Set 8 | GSM117726.CEL | tumor-enriched samples | U133P2 | 57 | 43 |
| Data Set 8 | GSM117727.CEL | tumor-enriched samples | U133P2 | 36 | 64 |
| Data Set 8 | GSM117741.CEL | tumor-enriched samples | U133P2 | 29 | 69 |
| Data Set 8 | GSM76640.CEL | tumor-enriched samples | U133P2 | 28 | 49 |
| Data Set 8 | GSM76648.CEL | tumor-enriched samples | U133P2 | 45 | 55 |
| Data Set 8 | GSM88977.CEL | tumor-enriched samples | U133P2 | 57 | 43 |
| Data Set 8 | GSM89017.CEL | tumor-enriched samples | U133P2 | 59 | 41 |
| Data Set 8 | GSM102435.CEL | tumor-enriched samples | U133P2 | 22 | 78 |
| Data Set 8 | GSM53061.CEL | tumor-enriched samples | U133P2 | 32 | 68 |
| Data Set 8 | GSM53114.CEL | tumor-enriched samples | U133P2 | 30 | 60 |
| Data Set 8 | GSM53152.CEL | tumor-enriched samples | U133P2 | 62 | 38 |
| Data Set 8 | GSM53162.CEL | tumor-enriched samples | U133P2 | 67 | 33 |
| Data Set 8 | GSM76516.CEL | tumor-enriched samples | U133P2 | 44 | 56 |
| Data Set 8 | GSM76544.CEL | tumor-enriched samples | U133P2 | 17 | 83 |
| Data Set 8 | GSM76553.CEL | tumor-enriched samples | U133P2 | 55 | 45 |
| Data Set 8 | GSM325799.CEL | tumor-enriched samples | U133P2 | 45 | 55 |
| Data Set 8 | GSM325802.CEL | tumor-enriched samples | U133P2 | 11 | 89 |
| Data Set 8 | GSM325804.CEL | tumor-enriched samples | U133P2 | 33 | 67 |
| Data Set 8 | GSM325810.CEL | tumor-enriched samples | U133P2 | 23 | 77 |
| Data Set 8 | GSM353882.CEL | tumor-enriched samples | U133P2 | 49 | 51 |
| Data Set 8 | GSM353884.CEL | tumor-enriched samples | U133P2 | 19 | 81 |
| Data Set 8 | GSM353891.CEL | tumor-enriched samples | U133P2 | 52 | 48 |
| Data Set 8 | GSM353892.CEL | tumor-enriched samples | U133P2 | 56 | 44 |
| Data Set 8 | GSM353893.CEL | tumor-enriched samples | U133P2 | 29 | 65 |
| Data Set 8 | GSM353894.CEL | tumor-enriched samples | U133P2 | 23 | 61 |
| Data Set 8 | GSM353899.CEL | tumor-enriched samples | U133P2 | 33 | 67 |
| Data Set 8 | GSM353910.CEL | tumor-enriched samples | U133P2 | 44 | 56 |
| Data Set 8 | GSM353917.CEL | tumor-enriched samples | U133P2 | 41 | 59 |
| Data Set 8 | GSM353940.CEL | tumor-enriched samples | U133P2 | 29 | 71 |
| Data Set 8 | GSM179901.CEL | tumor-enriched samples | U133P2 | 56 | 44 |
| Data Set 8 | GSM179903.CEL | tumor-enriched samples | U133P2 | 27 | 73 |
| Data Set 8 | GSM179954.CEL | tumor-enriched samples | U133P2 | 58 | 42 |
| Data Set 8 | GSM203677.CEL | tumor-enriched samples | U133P2 | 17 | 83 |
| Data Set 8 | GSM203707.CEL | tumor-enriched samples | U133P2 | 24 | 76 |
| Data Set 8 | GSM203711.CEL | tumor-enriched samples | U133P2 | 30 | 70 |
| Data Set 8 | GSM203715.CEL | tumor-enriched samples | U133P2 | 37 | 63 |
| Data Set 8 | GSM203722.CEL | tumor-enriched samples | U133P2 | 25 | 75 |
| Data Set 8 | GSM203740.CEL | tumor-enriched samples | U133P2 | 45 | 55 |
| Data Set 8 | GSM203764.CEL | tumor-enriched samples | U133P2 | 47 | 53 |
| Data Set 8 | GSM203778.CEL | tumor-enriched samples | U133P2 | 59 | 39 |
| Data Set 8 | GSM203786.CEL | tumor-enriched samples | U133P2 | 52 | 48 |
| Data Set 8 | GSM231872.CEL | tumor-enriched samples | U133P2 | 57 | 43 |
| Data Set 8 | GSM231876.CEL | tumor-enriched samples | U133P2 | 10 | 90 |
| Data Set 8 | GSM231881.CEL | tumor-enriched samples | U133P2 | 24 | 76 |
| Data Set 8 | GSM231888.CEL | tumor-enriched samples | U133P2 | 28 | 72 |
| Data Set 8 | GSM231894.CEL | tumor-enriched samples | U133P2 | 30 | 70 |
| Data Set 8 | GSM231944.CEL | tumor-enriched samples | U133P2 | 37 | 63 |
| Data Set 8 | GSM231951.CEL | tumor-enriched samples | U133P2 | 23 | 57 |
| Data Set 8 | GSM231957.CEL | tumor-enriched samples | U133P2 | 57 | 43 |
| Data Set 8 | GSM231978.CEL | tumor-enriched samples | U133P2 | 41 | 59 |
| Data Set 8 | GSM231979.CEL | tumor-enriched samples | U133P2 | 36 | 57 |
| Data Set 8 | GSM231990.CEL | tumor-enriched samples | U133P2 | 29 | 71 |
| Data Set 8 | GSM277677.CEL | tumor-enriched samples | U133P2 | 12 | 82 |
| Data Set 8 | GSM277683.CEL | tumor-enriched samples | U133P2 | 55 | 45 |
| Data Set 8 | GSM277694.CEL | tumor-enriched samples | U133P2 | 40 | 60 |
| Data Set 8 | GSM301659.CEL | tumor-enriched samples | U133P2 | 15 | 85 |
| Data Set 8 | GSM301665.CEL | tumor-enriched samples | U133P2 | 3 | 78 |
| Data Set 8 | GSM301666.CEL | tumor-enriched samples | U133P2 | 14 | 66 |
| Data Set 8 | GSM301670.CEL | tumor-enriched samples | U133P2 | 30 | 70 |
| Data Set 8 | GSM301674.CEL | tumor-enriched samples | U133P2 | 16 | 84 |
| Data Set 8 | GSM301679.CEL | tumor-enriched samples | U133P2 | 42 | 58 |
| Data Set 8 | GSM301701.CEL | tumor-enriched samples | U133P2 | 34 | 66 |
| Data Set 8 | GSM301709.CEL | tumor-enriched samples | U133P2 | 46 | 54 |
| Data Set 8 | GSM38053.CEL | tumor-enriched samples | U133P2 | 39 | 61 |

TABLE 18
Genes identified by permutation strategy to select the most suitable genes for the final prediction model

| DataSet | geneModel | uniqueID | Gene Symbol | Gene Description |
|---|---|---|---|---|
| Data Set 1 | 5 gene model | 202555_s_at | MYLK | myosin, light polypeptide kinase /// myosin, light polypeptide kinase |
| Data Set 1 | 5 gene model | 219360_s_at | TRPM4 | transient receptor potential cation channel, subfamily M, member 4 |
| Data Set 1 | 5 gene model | 209825_s_at | UCK2 | uridine-cytidine kinase 2 |
| Data Set 1 | 5 gene model | 204973_at | GJB1 | gap junction protein, beta 1, 32 kDa (connexin 32, Charcot-Marie-Tooth |
| | | | | neuropathy, X-linked) |
| Data Set 1 | 5 gene model | 214027_x_at | DES /// FAM48A | desmin /// family with sequence similarity 48, member A |
| Data Set 1 | 10 gene model | 202222_s_at | DES | desmin |
| Data Set 1 | 10 gene model | 205547_s_at | TAGLN | transgelin |
| Data Set 1 | 10 gene model | 203766_s_at | LMOD1 | leiomodin 1 (smooth muscle) |
| Data Set 1 | 10 gene model | 217728_at | S100A6 | S100 calcium binding protein A6 (calcyclin) |
| Data Set 1 | 10 gene model | 209825_s_at | UCK2 | uridine-cytidine kinase 2 |
| Data Set 1 | 10 gene model | 208792_s_at | CLU | clusterin (complement lysis inhibitor, SP-40,40, sulfated glycoprotein 2, |
| | | | | testosterone-repressed prostate message 2, apolipoprotein J) |
| Data Set 1 | 10 gene model | 212412_at | PDLIM5 | PDZ and LIM domain 5 |
| Data Set 1 | 10 gene model | 219360_s_at | TRPM4 | transient receptor potential cation channel, subfamily M, member 4 |
| Data Set 1 | 10 gene model | 201061_s_at | STOM | stomatin |
| Data Set 1 | 10 gene model | 209283_at | CRYAB | crystallin, alpha B |
| Data Set 1 | 20 gene model | 200982_s_at | ANXA6 | annexin A6 |
| Data Set 1 | 20 gene model | 218094_s_at | C20orf35 | chromosome 20 open reading frame 35 |
| Data Set 1 | 20 gene model | 203951_at | CNN1 | calponin 1, basic, smooth muscle |
| Data Set 1 | 20 gene model | 209356_x_at | EFEMP2 | EGF-containing fibulin-like extracellular matrix protein 2 |
| Data Set 1 | 20 gene model | 206580_s_at | EFEMP2 | EGF-containing fibulin-like extracellular matrix protein 2 |
| Data Set 1 | 20 gene model | 201590_x_at | ANXA2 | annexin A2 |
| Data Set 1 | 20 gene model | 219167_at | RASL12 | RAS-like, family 12 |
| Data Set 1 | 20 gene model | 201105_at | LGALS1 | lectin, galactoside-binding, soluble, 1 (galectin 1) |
| Data Set 1 | 20 gene model | 206558_at | SIM2 | single-minded homolog 2 (Drosophila) |
| Data Set 1 | 20 gene model | 217728_at | S100A6 | S100 calcium binding protein A6 (calcyclin) |
| Data Set 1 | 20 gene model | 202148_s_at | PYCR1 | pyrroline-5-carboxylate reductase 1 |
| Data Set 1 | 20 gene model | 205547_s_at | TAGLN | transgelin |
| Data Set 1 | 20 gene model | 209825_s_at | UCK2 | uridine-cytidine kinase 2 |
| Data Set 1 | 20 gene model | 212412_at | PDLIM5 | PDZ and LIM domain 5 |
| Data Set 1 | 20 gene model | 209283_at | CRYAB | crystallin, alpha B |
| Data Set 1 | 20 gene model | 205645_at | REPS2 | RALBP1 associated Eps domain containing 2 |
| Data Set 1 | 20 gene model | 203766_s_at | LMOD1 | leiomodin 1 (smooth muscle) |
| Data Set 1 | 20 gene model | 208792_s_at | CLU | clusterin (complement lysis inhibitor, SP-40,40, sulfated glycoprotein 2 |
| | | | | testosterone-repressed prostate message 2, apolipoprotein J) |
| Data Set 1 | 20 gene model | 201061_s_at | STOM | stomatin |
| Data Set 1 | 20 gene model | 201820_at | KRT5 | keratin 5 (epidermolysis bullosa simplex, Dowling-Meara/Kobner/Weber- |
| | | | | Cockayne types) |
| Data Set 1 | 50 gene model | 200621_at | CSRP1 | cysteine and glycine-rich protein 1 |
| Data Set 1 | 50 gene model | 212236_x_at | KRT17 | keratin 17 |
| Data Set 1 | 50 gene model | 205856_at | SLC14A1 | solute carrier family 14 (urea transporter), member 1 (Kidd blood group) |
| Data Set 1 | 50 gene model | 207949_s_at | ICA1 | islet cell autoantigen 1, 69 kDa |
| Data Set 1 | 50 gene model | 205505_at | GCNT1 | glucosaminyl (N-acetyl) transferase 1, core 2 (beta-1,6-N-acetylglucosa- |
| | | | | minyltransferase) |
| Data Set 1 | 50 gene model | 205935_at | FOXF1 | forkhead box F1 |
| Data Set 1 | 50 gene model | 213503_x_at | ANXA2 | annexin A2 |
| Data Set 1 | 50 gene model | 210427_x_at | ANXA2 | annexin A2 |
| Data Set 1 | 50 gene model | 208816_x_at | ANXA2P2 | annexin A2 pseudogene 2 |
| Data Set 1 | 50 gene model | 203638_s_at | FGFR2 | fibroblast growth factor receptor 2 (bacteria-expressed kinase, |
| | | | | keratinocyte growth factor receptor, craniofacial dysostosis 1, Crouzon |
| | | | | syndrome, Pfeiffer syndrome, Jackson-Weiss syndrome) |
| Data Set 1 | 50 gene model | 203892_at | WFDC2 | WAP four-disulfide core domain 2 |
| Data Set 1 | 50 gene model | 210986_s_at | TPM1 | tropomyosin 1 (alpha) |
| Data Set 1 | 50 gene model | 202565_s_at | SVIL | supervillin |
| Data Set 1 | 50 gene model | 203228_at | PAFAH1B3 | platelet-activating factor acetylhydrolase, isoform Ib, gamma subunit 29 kDa |
| Data Set 1 | 50 gene model | 213288_at | OACT2 | O-acyltransferase (membrane bound) domain containing 2 |
| Data Set 1 | 50 gene model | 204394_at | SLC43A1 | solute carrier family 43, member 1 |
| Data Set 1 | 50 gene model | 203243_s_at | PDLIM5 | PDZ and LIM domain 5 |
| Data Set 1 | 50 gene model | 201431_s_at | DPYSL3 | dihydropyrimidinase-like 3 |
| Data Set 1 | 50 gene model | 219736_at | TRIM36 | tripartite motif-containing 36 |
| Data Set 1 | 50 gene model | 201058_s_at | MYL9 | myosin, light polypeptide 9, regulatory |
| Data Set 1 | 50 gene model | 212509_s_at | MXRA7 | matrix-remodelling associated 7 |
| Data Set 1 | 50 gene model | 46323_at | CANT1 | calcium activated nucleotidase 1 |
| Data Set 1 | 50 gene model | 205309_at | SMPDL3B | sphingomyelin phosphodiesterase, acid-like 3B |
| Data Set 1 | 50 gene model | 209545_s_at | RIPK2 | receptor-interacting serine-threonine kinase 2 |
| Data Set 1 | 50 gene model | 209763_at | CHRDL1 | chordin-like 1 |
| Data Set 1 | 50 gene model | 205687_at | UBPH | ubiquitin-binding protein homolog |
| Data Set 1 | 50 gene model | 202283_at | SERPINF1 | serpin peptidase inhibitor, clade F (alpha-2 antiplasmin, pigment |
| | | | | epithelium derived factor), member 1 |
| Data Set 1 | 50 gene model | 203323_at | CAV2 | caveolin 2 |
| Data Set 1 | 50 gene model | 210869_s_at | MCAM | melanoma cell adhesion molecule |
| Data Set 1 | 50 gene model | 212116_at | RFP | ret finger protein |
| Data Set 1 | 50 gene model | 221732_at | CANT1 | calcium activated nucleotidase 1 |
| Data Set 1 | 50 gene model | 219478_at | WFDC1 | WAP four-disulfide core domain 1 |
| Data Set 1 | 50 gene model | 218865_at | MOSC1 | MOCO sulphurase C-terminal domain containing 1 |
| Data Set 1 | 50 gene model | 200897_s_at | KIAA0992 | palladin |
| Data Set 1 | 50 gene model | 203632_s_at | GPRC5B | G protein-coupled receptor, family C, group 5, member B |
| Data Set 1 | 50 gene model | 211576_s_at | SLC19A1 | solute carrier family 19 (folate transporter), member 1 |
| Data Set 1 | 50 gene model | 212886_at | DKFZP434C171 | DKFZP434C171 protein |
| Data Set 1 | 50 gene model | 202949_s_at | FHL2 | four and a half LIM domains 2 |
| Data Set 1 | 50 gene model | 208690_s_at | PDLIM1 | PDZ and LIM domain 1 (elfin) |
| Data Set 1 | 50 gene model | 217912_at | DUS1L | dihydrouridine synthase 1-like (S. cerevisiae) |
| Data Set 1 | 50 gene model | 206580_s_at | EFEMP2 | EGF-containing fibulin-like extracellular matrix protein 2 |
| Data Set 1 | 50 gene model | 212097_at | CAV1 | caveolin 1, caveolae protein, 22 kDa |
| Data Set 1 | 50 gene model | 202274_at | ACTG2 | actin, gamma 2, smooth muscle, enteric |
| Data Set 1 | 50 gene model | 212813_at | JAM3 | junctional adhesion molecule 3 |
| Data Set 1 | 50 gene model | 201105_at | LGALS1 | lectin, galactoside-binding, soluble, 1 (galectin 1) |
| Data Set 1 | 50 gene model | 201014_s_at | PAICS | phosphoribosylaminoimidazole carboxylase, phosphoribosyl- |
| | | | | aminoimidazole succinocarboxamide synthetase |
| Data Set 1 | 50 gene model | 206558_at | SIM2 | single-minded homolog 2 (Drosophila) |
| Data Set 1 | 50 gene model | 202440_s_at | ST5 | suppression of tumorigenicity 5 |
| Data Set 1 | 50 gene model | 200795_at | SPARCL1 | SPARC-like 1 (mast9, hevin) |
| Data Set 1 | 50 gene model | 212724_at | RND3 | Rho family GTPase 3 |
| Data Set 1 | 100 gene model | 202740_at | ACY1 | aminoacylase 1 |
| Data Set 1 | 100 gene model | 204400_at | EFS | embryonal Fyn-associated substrate |
| Data Set 1 | 100 gene model | 204570_at | COX7A1 | cytochrome c oxidase subunit VIIa polypeptide 1 (muscle) |
| Data Set 1 | 100 gene model | 201272_at | AKR1B1 | aldo-keto reductase family 1, member B1 (aldose reductase) |
| Data Set 1 | 100 gene model | 201284_s_at | APEH | N-acylaminoacyl-peptide hydrolase |
| Data Set 1 | 100 gene model | 214156_at | MYRIP | myosin VIIA and Rab interacting protein |
| Data Set 1 | 100 gene model | 203562_at | FEZ1 | fasciculation and elongation protein zeta 1 (zygin I) |
| Data Set 1 | 100 gene model | 209170_s_at | GPM6B | glycoprotein M6B |
| Data Set 1 | 100 gene model | 202429_s_at | PPP3CA | protein phosphatase 3 (formerly 2B), catalytic subunit, alpha |
| | | | | isoform (calcineurin A alpha) |
| Data Set 1 | 100 gene model | 212680_x_at | PPP1R14B | protein phosphatase 1, regulatory (inhibitor) subunit 14B |
| Data Set 1 | 100 gene model | 213996_at | YPEL1 | yippee-like 1 (Drosophila) |
| Data Set 1 | 100 gene model | 200700_s_at | KDELR2 | KDEL (Lys-Asp-Glu-Leu) endoplasmic reticulum protein |
| | | | | retention receptor 2 |
| Data Set 1 | 100 gene model | 216565_x_at | LOC391020 | similar to Interferon-induced transmembrane protein 3 (Interferon- |
| | | | | inducible protein 1-8U) |
| Data Set 1 | 100 gene model | 213001_at | ANGPTL2 | angiopoietin-like 2 |
| Data Set 1 | 100 gene model | 221586_s_at | E2F5 | E2F transcription factor 5, p130-binding |
| Data Set 1 | 100 gene model | 200971_s_at | SERP1 | stress-associated endoplasmic reticulum protein 1 |
| Data Set 1 | 100 gene model | 200923_at | LGALS3BP | lectin, galactoside-binding, soluble, 3 binding protein |
| Data Set 1 | 100 gene model | 202073_at | OPTN | optineurin |
| Data Set 1 | 100 gene model | 203498_at | DSCR1L1 | Down syndrome critical region gene 1-like 1 |
| Data Set 1 | 100 gene model | 206860_s_at | FLJ20323 | hypothetical protein FLJ20323 |
| Data Set 1 | 100 gene model | 217973_at | DCXR | dicarbonyl/L-xylulose reductase |
| Data Set 1 | 100 gene model | 209616_s_at | CES1 | carboxylesterase 1 (monocyte/macrophage serine esterase 1) |
| Data Set 1 | 100 gene model | 204754_at | HLF | Hepatic leukemia factor |
| Data Set 1 | 100 gene model | 209550_at | NDN | necdin homolog (mouse) |
| Data Set 1 | 100 gene model | 208131_s_at | PTGIS | prostaglandin I2 (prostacyclin) synthase /// prostaglandin I2 |
| | | | | (prostacyclin) synthase |
| Data Set 1 | 100 gene model | 203729_at | EMP3 | epithelial membrane protein 3 |
| Data Set 1 | 100 gene model | 203892_at | WFDC2 | WAP four-disulfide core domain 2 |
| Data Set 1 | 100 gene model | 202794_at | INPP1 | inositol polyphosphate-1-phosphatase |
| Data Set 1 | 100 gene model | 209210_s_at | PLEKHC1 | pleckstrin homology domain containing, family C (with FERM |
| | | | | domain) member 1 |
| Data Set 1 | 100 gene model | 209191_at | TUBB6 | tubulin, beta 6 |
| Data Set 1 | 100 gene model | 217897_at | FXYD6 | FXYD domain containing ion transport regulator 6 |
| Data Set 1 | 100 gene model | 209434_s_at | PPAT | phosphoribosyl pyrophosphate amidotransferase |
| Data Set 1 | 100 gene model | 202427_s_at | BRP44 | brain protein 44 |
| Data Set 1 | 100 gene model | 204041_at | MAOB | monoamine oxidase B |
| Data Set 1 | 100 gene model | 202177_at | GAS6 | growth arrest-specific 6 |
| Data Set 1 | 100 gene model | 212067_s_at | C1R | complement component 1, r subcomponent |
| Data Set 1 | 100 gene model | 214247_s_at | DKK3 | dickkopf homolog 3 (Xenopus laevis) |
| Data Set 1 | 100 gene model | 205780_at | BIK | BCL2-interacting killer (apoptosis-inducing) |
| Data Set 1 | 100 gene model | 205776_at | FMO5 | flavin containing monooxygenase 5 |
| Data Set 1 | 100 gene model | 220192_x_at | SPDEF | SAM pointed domain containing ets transcription factor |
| Data Set 1 | 100 gene model | 218922_s_at | LASS4 | LAG1 longevity assurance homolog 4 (S. cerevisiae) |
| Data Set 1 | 100 gene model | 200907_s_at | KIAA0992 | palladin |
| Data Set 1 | 100 gene model | 207836_s_at | RBPMS | RNA binding protein with multiple splicing |
| Data Set 1 | 100 gene model | 203638_s_at | FGFR2 | fibroblast growth factor receptor 2 (bacteria-expressed kinase, |
| | | | | keratinocyte growth factor receptor, craniofacial dysostosis 1, |
| | | | | Crouzon syndrome, Pfeiffer syndrome, Jackson-Weiss syndrome) |
| Data Set 1 | 100 gene model | 203242_s_at | PDLIM5 | PDZ and LIM domain 5 |
| Data Set 1 | 100 gene model | 209624_s_at | MCCC2 | methylcrotonoyl-Coenzyme A carboxylase 2 (beta) |
| Data Set 1 | 100 gene model | 212736_at | C16orf45 | chromosome 16 open reading frame 45 |
| Data Set 1 | 100 gene model | 206116_s_at | TPM1 | tropomyosin 1 (alpha) |
| Data Set 1 | 100 gene model | 212843_at | NCAM1 | neural cell adhesion molecule 1 |
| Data Set 1 | 100 gene model | 202947_s_at | GYPC | glycophorin C (Gerbich blood group) |
| Data Set 1 | 100 gene model | 207876_s_at | FLNC | filamin C, gamma (actin binding protein 280) |
| Data Set 1 | 100 gene model | 204069_at | MEIS1 | Meis1, myeloid ecotropic viral integration site 1 homolog (mouse) |
| Data Set 1 | 100 gene model | 209087_x_at | MCAM | melanoma cell adhesion molecule |
| Data Set 1 | 100 gene model | 212236_x_at | KRT17 | keratin 17 |
| Data Set 1 | 100 gene model | 204394_at | SLC43A1 | solute carrier family 43, member 1 |
| Data Set 1 | 100 gene model | 212115_at | C16orf34 | chromosome 16 open reading frame 34 |
| Data Set 1 | 100 gene model | 202074_s_at | OPTN | optineurin |
| Data Set 1 | 100 gene model | 222043_at | CLU | clusterin (complement lysis inhibitor, SP-40,40, sulfated glycoprotein |
| | | | | 2, testosterone-repressed prostate message 2, apolipoprotein J) |
| Data Set 1 | 100 gene model | 206858_s_at | HOXC6 | homeo box C6 |
| Data Set 1 | 100 gene model | 218418_s_at | ANKRD25 | ankyrin repeat domain 25 |
| Data Set 1 | 100 gene model | 213924_at | MPPE1 | Metallophosphoesterase 1 |
| Data Set 1 | 100 gene model | 202504_at | TRIM29 | tripartite motif-containing 29 |
| Data Set 1 | 100 gene model | 205937_at | CGREF1 | cell growth regulator with EF-hand domain 1 |
| Data Set 1 | 100 gene model | 208837_at | TMED3 | transmembrane emp24 protein transport domain containing 3 |
| Data Set 1 | 100 gene model | 216804_s_at | PDLIM5 | PDZ and LIM domain 5 |
| Data Set 1 | 100 gene model | 203911_at | RAP1GA1 | RAP1, GTPase activating protein 1 |
| Data Set 1 | 100 gene model | 210299_s_at | FHL1 | four and a half LIM domains 1 |
| Data Set 1 | 100 gene model | 210427_x_at | ANXA2 | annexin A2 |
| Data Set 1 | 100 gene model | 210987_x_at | TPM1 | tropomyosin 1 (alpha) |
| Data Set 1 | 100 gene model | 210243_s_at | B4GALT3 | UDP-Gal:betaGlcNAc beta 1,4-galactosyltransferase, polypeptide 3 |
| Data Set 1 | 100 gene model | 209665_at | CYB561D2 | cytochrome b-561 domain containing 2 |
| Data Set 1 | 100 gene model | 210986_s_at | TPM1 | tropomyosin 1 (alpha) |
| Data Set 1 | 100 gene model | 203243_s_at | PDLIM5 | PDZ and LIM domain 5 |
| Data Set 1 | 100 gene model | 205856_at | SLC14A1 | solute carrier family 14 (urea transporter), member 1 (Kidd blood group) |
| Data Set 1 | 100 gene model | 200974_at | ACTA2 | actin, alpha 2, smooth muscle, aorta |
| Data Set 1 | 100 gene model | 202283_at | SERPINF1 | serpin peptidase inhibitor, clade F (alpha-2 antiplasmin, pigment epithelium |
| | | | | derived factor), member 1 |
| Data Set 1 | 100 gene model | 209545_s_at | RIPK2 | receptor-interacting serine-threonine kinase 2 |
| Data Set 1 | 100 gene model | 203228_at | PAFAH1B3 | platelet-activating factor acetylhydrolase, isoform Ib, gamma subunit 29 kDa |
| Data Set 1 | 100 gene model | 201058_s_at | MYL9 | myosin, light polypeptide 9, regulatory |
| Data Set 1 | 100 gene model | 205309_at | SMPDL3B | sphingomyelin phosphodiesterase, acid-like 3B |
| Data Set 1 | 100 gene model | 212116_at | RFP | ret finger protein |
| Data Set 1 | 100 gene model | 212509_s_at | MXRA7 | matrix-remodelling associated 7 |
| Data Set 1 | 100 gene model | 209118_s_at | TUBA3 | tubulin, alpha 3 |
| Data Set 1 | 100 gene model | 202565_s_at | SVIL | supervillin |
| Data Set 1 | 100 gene model | 218865_at | MOSC1 | MOCO sulphurase C-terminal domain containing 1 |
| Data Set 1 | 100 gene model | 203632_s_at | GPRC5B | G protein-coupled receptor, family C, group 5, member B |
| Data Set 1 | 100 gene model | 201431_s_at | DPYSL3 | dihydropyrimidinase-like 3 |
| Data Set 1 | 100 gene model | 207949_s_at | ICA1 | islet cell autoantigen 1, 69 kDa |
| Data Set 1 | 100 gene model | 209948_at | KCNMB1 | potassium large conductance calcium-activated channel, subfamily M, |
| | | | | beta member 1 |
| Data Set 1 | 100 gene model | 209426_s_at | AMACR | alpha-methylacyl-CoA racemase |
| Data Set 1 | 100 gene model | 209424_s_at | AMACR | alpha-methylacyl-CoA racemase |
| Data Set 1 | 100 gene model | 209425_at | AMACR | alpha-methylacyl-CoA racemase |
| Data Set 1 | 100 gene model | 204083_s_at | TPM2 | tropomyosin 2 (beta) |
| Data Set 1 | 100 gene model | 204934_s_at | HPN | hepsin (transmembrane protease, serine 1) |
| Data Set 1 | 100 gene model | 211276_at | TCEAL2 | transcription elongation factor A (SII)-like 2 |
| Data Set 1 | 100 gene model | 201061_s_at | STOM | stomatin |
| Data Set 1 | 100 gene model | 204973_at | GJB1 | gap junction protein, beta 1, 32 kDa (connexin 32, Charcot-Marie-Tooth |
| | | | | neuropathy, X-linked) |
| Data Set 1 | 100 gene model | 200824_at | GSTP1 | glutathione S-transferase pi |
| Data Set 1 | 100 gene model | 202555_s_at | MYLK | myosin, light polypeptide kinase /// myosin, light polypeptide kinase |
| Data Set 1 | 100 gene model | 214027_x_at | DES /// FAM48A | desmin /// family with sequence similarity 48, member A |
| Data Set 1 | 250 gene model | 222199_s_at | BIN3 | bridging integrator 3 |
| Data Set 1 | 250 gene model | 209623_at | MCCC2 | methylcrotonoyl-Coenzyme A carboxylase 2 (beta) |
| Data Set 1 | 250 gene model | 202889_x_at | MAP7 | microtubule-associated protein 7 |
| Data Set 1 | 250 gene model | 200862_at | DHCR24 | 24-dehydrocholesterol reductase |
| Data Set 1 | 250 gene model | 217736_s_at | EIF2AK1 | eukaryotic translation initiation factor 2-alpha kinase 1 |
| Data Set 1 | 250 gene model | 209813_x_at | TRGC2 /// TRGV9 | T cell receptor gamma constant 2 /// T cell receptor gamma constant 2 /// |
| | | | /// LOC442532 /// | T cell receptor gamma variable 9 /// T cell receptor gamma variable 9 /// |
| | | | LOC442670 /// | similar to T-cell receptor gamma chain C region PT-gamma-1/2 /// similar |
| | | | TARP | to T-cell receptor gamma chain C region PT-gamma-1/2 /// similar to T-cell |
| | | | | receptor gamma chain V region PT-gamma-1/2 precursor /// similar to T-cell |
| | | | | receptor gamma chain V region PT-gamma-1/2 precursor /// TCR gamma |
| | | | | alternate reading frame protein /// TCR gamma alternate reading frame protein |
| Data Set 1 | 250 gene model | 215806_x_at | TRGC2 /// TRGV9 | T cell receptor gamma constant 2 /// T cell receptor gamma variable 9 /// |
| | | | /// LOC442532 /// | similar to T-cell receptor gamma chain C region PT-gamma-1/2 /// similar to |
| | | | LOC442670 /// | T-cell receptor gamma chain V region PT-gamma-1/2 precursor /// TCR |
| | | | TARP | gamma alternate reading frame protein |
| Data Set 1 | 250 gene model | 222121_at | SGEF | Src homology 3 domain-containing guanine nucleotide exchange factor |
| Data Set 1 | 250 gene model | 216920_s_at | TRGC2 /// TRGV9 | T cell receptor gamma constant 2 /// T cell receptor gamma variable 9 |
| | | | /// LOC442532 /// | /// similar to T-cell receptor gamma chain C region PT-gamma-1/2 /// |
| | | | LOC442670 /// | similar to T-cell receptor gamma chain V region PT-gamma-1/2 precursor |
| | | | TARP | /// TCR gamma alternate reading frame protein |
| Data Set 1 | 250 gene model | 202729_s_at | LTBP1 | latent transforming growth factor beta binding protein 1 |
| Data Set 1 | 250 gene model | 204667_at | FOXA1 | forkhead box A1 |
| Data Set 1 | 250 gene model | 209584_x_at | APOBEC3C | apolipoprotein B mRNA editing enzyme, catalytic polypeptide-like 3C |
| Data Set 1 | 250 gene model | 203662_s_at | TMOD1 | tropomodulin 1 |
| Data Set 1 | 250 gene model | 203629_s_at | COG5 | component of oligomeric golgi complex 5 |
| Data Set 1 | 250 gene model | 201839_s_at | TACSTD1 | tumor-associated calcium signal transducer 1 |
| Data Set 1 | 250 gene model | 201128_s_at | ACLY | ATP citrate lyase |
| Data Set 1 | 250 gene model | 214106_s_at | GMDS | GDP-mannose 4,6-dehydratase |
| Data Set 1 | 250 gene model | 210224_at | MR1 | major histocompatibility complex, class I-related |
| Data Set 1 | 250 gene model | 202071_at | SDC4 | syndecan 4 (amphiglycan, ryudocan) |
| Data Set 1 | 250 gene model | 214733_s_at | YIPF1 | Yip1 domain family, member 1 |
| Data Set 1 | 250 gene model | 219806_s_at | FN5 | FN5 protein |
| Data Set 1 | 250 gene model | 213506_at | F2RL1 | coagulation factor II (thrombin) receptor-like 1 |
| Data Set 1 | 250 gene model | 221565_s_at | FAM26B | family with sequence similarity 26, member B |
| Data Set 1 | 250 gene model | 219920_s_at | GMPPB | GDP-mannose pyrophosphorylase B |
| Data Set 1 | 250 gene model | 221027_s_at | PLA2G12A | phospholipase A2, group XIIA /// phospholipase A2, group XIIA |
| Data Set 1 | 250 gene model | 209086_x_at | MCAM | melanoma cell adhesion molecule |
| Data Set 1 | 250 gene model | 207957_s_at | PRKCB1 | Protein kinase C, beta 1 |
| Data Set 1 | 250 gene model | 221880_s_at | LOC400451 | hypothetical gene supported by AK075564; BC060873 |
| Data Set 1 | 250 gene model | 221669_s_at | ACAD8 | acyl-Coenzyme A dehydrogenase family, member 8 |
| Data Set 1 | 250 gene model | 205248_at | C21orf5 | chromosome 21 open reading frame 5 |
| Data Set 1 | 250 gene model | 206656_s_at | C20orf3 | chromosome 20 open reading frame 3 |
| Data Set 1 | 250 gene model | 202566_s_at | SVIL | supervillin |
| Data Set 1 | 250 gene model | 214765_s_at | ASAHL | N-acylsphingosine amidohydrolase (acid ceramidase)-like |
| Data Set 1 | 250 gene model | 210652_s_at | C1orf34 | chromosome 1 open reading frame 34 |
| Data Set 1 | 250 gene model | 202202_s_at | LAMA4 | laminin, alpha 4 |
| Data Set 1 | 250 gene model | 201605_x_at | CNN2 | calponin 2 |
| Data Set 1 | 250 gene model | 212551_at | CAP2 | CAP, adenylate cyclase-associated protein, 2 (yeast) |
| Data Set 1 | 250 gene model | 201136_at | PLP2 | proteolipid protein 2 (colonic epithelium-enriched) |
| Data Set 1 | 250 gene model | 218328_at | COQ4 | coenzyme Q4 homolog (yeast) |
| Data Set 1 | 250 gene model | 219786_at | MTL5 | metallothionein-like 5, testis-specific (tesmin) |
| Data Set 1 | 250 gene model | 206375_s_at | HSPB3 | heat shock 27 kDa protein 3 |
| Data Set 1 | 250 gene model | 212563_at | BOP1 | block of proliferation 1 |
| Data Set 1 | 250 gene model | 218792_s_at | BSPRY | B-box and SPRY domain containing |
| Data Set 1 | 250 gene model | 209270_at | LAMB3 | laminin, beta 3 |
| Data Set 1 | 250 gene model | 221898_at | PDPN | podoplanin |
| Data Set 1 | 250 gene model | 206110_at | HIST1H3H | histone 1, H3h |
| Data Set 1 | 250 gene model | 213547_at | CAND2 | cullin-associated and neddylation-dissociated 2 (putative) |
| Data Set 1 | 250 gene model | 204345_at | COL16A1 | collagen, type XVI, alpha 1 |
| Data Set 1 | 250 gene model | 208579_x_at | H2BFS | H2B histone family, member S |
| Data Set 1 | 250 gene model | 205850_s_at | GABRB3 | gamma-aminobutyric acid (GABA) A receptor, beta 3 |
| Data Set 1 | 250 gene model | 205304_s_at | KCNJ8 | potassium inwardly-rectifying channel, subfamily J, member 8 |
| Data Set 1 | 250 gene model | 201284_s_at | APEH | N-acylaminoacyl-peptide hydrolase |
| Data Set 1 | 250 gene model | 208490_x_at | HIST1H2BF | histone 1, H2bf |
| Data Set 1 | 250 gene model | 218944_at | PYCRL | pyrroline-5-carboxylate reductase-like |
| Data Set 1 | 250 gene model | 209154_at | TAX1BP3 | Tax1 (human T-cell leukemia virus type I) binding protein 3 |
| Data Set 1 | 250 gene model | 215380_s_at | C7orf24 | chromosome 7 open reading frame 24 |
| Data Set 1 | 250 gene model | 219517_at | ELL3 | elongation factor RNA polymerase II-like 3 |
| Data Set 1 | 250 gene model | 213275_x_at | CTSB | cathepsin B |
| Data Set 1 | 250 gene model | 201300_s_at | PRNP | prion protein (p27-30) (Creutzfeld-Jakob disease, Gerstmann- |
| | | | | Strausler-Scheinker syndrome, fatal familial insomnia) |
| Data Set 1 | 250 gene model | 204294_at | AMT | aminomethyltransferase (glycine cleavage system protein T) |
| Data Set 1 | 250 gene model | 219935_at | ADAMTS5 | ADAM metallopeptidase with thrombospondin type 1 motif, 5 |
| | | | | (aggrecanase-2) |
| Data Set 1 | 250 gene model | 201030_x_at | LDHB | lactate dehydrogenase B |
| Data Set 1 | 250 gene model | 217890_s_at | PARVA | parvin, alpha |
| Data Set 1 | 250 gene model | 213148_at | LOC257407 | hypothetical protein LOC257407 |
| Data Set 1 | 250 gene model | 203931_s_at | MRPL12 | mitochondrial ribosomal protein L12 |
| Data Set 1 | 250 gene model | 214077_x_at | MEIS4 | Meis1, myeloid ecotropic viral integration site 1 homolog 4 (mouse) |
| Data Set 1 | 250 gene model | 221505_at | ANP32E | acidic (leucine-rich) nuclear phosphoprotein 32 family, member E |
| Data Set 1 | 250 gene model | 218087_s_at | SORBS1 | sorbin and SH3 domain containing 1 |
| Data Set 1 | 250 gene model | 217764_s_at | RAB31 | RAB31, member RAS oncogene family |
| Data Set 1 | 250 gene model | 205011_at | LOH11CR2A | loss of heterozygosity, 11, chromosomal region 2, gene A |
| Data Set 1 | 250 gene model | 213293_s_at | TRIM22 | tripartite motif-containing 22 |
| Data Set 1 | 250 gene model | 204231_s_at | FAAH | fatty acid amide hydrolase |
| Data Set 1 | 250 gene model | 200878_at | EPAS1 | endothelial PAS domain protein 1 |
| Data Set 1 | 250 gene model | 203296_s_at | ATP1A2 | ATPase, Na+/K+ transporting, alpha 2 (+) polypeptide |
| Data Set 1 | 250 gene model | 202724_s_at | FOXO1A | forkhead box O1A (rhabdomyosarcoma) |
| Data Set 1 | 250 gene model | 201952_at | ALCAM | activated leukocyte cell adhesion molecule |
| Data Set 1 | 250 gene model | 208658_at | PDIA4 | protein disulfide isomerase family A, member 4 |
| Data Set 1 | 250 gene model | 203857_s_at | PDIA5 | protein disulfide isomerase family A, member 5 |
| Data Set 1 | 250 gene model | 219395_at | RBM35B | RNA binding motif protein 35B |
| Data Set 1 | 250 gene model | 209776_s_at | SLC19A1 | solute carrier family 19 (folate transporter), member 1 |
| Data Set 1 | 250 gene model | 209806_at | HIST1H2BK | histone 1, H2bk |
| Data Set 1 | 250 gene model | 211144_x_at | TRGC2 | T cell receptor gamma constant 2 |
| Data Set 1 | 250 gene model | 216905_s_at | ST14 | suppression of tumorigenicity 14 (colon carcinoma, matriptase, epithin) |
| Data Set 1 | 250 gene model | 218275_at | SLC25A10 | solute carrier family 25 (mitochondrial carrier; dicarboxylate |
| | | | | transporter), member 10 |
| Data Set 1 | 250 gene model | 203921_at | CHST2 | carbohydrate (N-acetylglucosamine-6-O) sulfotransferase 2 |
| Data Set 1 | 250 gene model | 202429_s_at | PPP3CA | protein phosphatase 3 (formerly 2B), catalytic subunit, alpha isoform |
| | | | | (calcineurin A alpha) |
| Data Set 1 | 250 gene model | 201185_at | HTRA1 | HtrA serine peptidase 1 |
| Data Set 1 | 250 gene model | 204141_at | TUBB2 | tubulin, beta 2 |
| Data Set 1 | 250 gene model | 219561_at | COPZ2 | coatomer protein complex, subunit zeta 2 |
| Data Set 1 | 250 gene model | 204123_at | LIG3 | ligase III, DNA, ATP-dependent |
| Data Set 1 | 250 gene model | 204777_s_at | MAL | mal, T-cell differentiation protein |
| Data Set 1 | 250 gene model | 205157_s_at | KRT17 | keratin 17 |
| Data Set 1 | 250 gene model | 212347_x_at | MXD4 | MAX dimerization protein 4 |
| Data Set 1 | 250 gene model | 213143_at | LOC257407 | hypothetical protein LOC257407 |
| Data Set 1 | 250 gene model | 202920_at | ANK2 | ankyrin 2, neuronal |
| Data Set 1 | 250 gene model | 217551_at | LOC441453 | similar to olfactory receptor, family 7, subfamily A, member 17 |
| Data Set 1 | 250 gene model | 212233_at | MAP1B | Microtubule-associated protein 1B /// Homo sapiens, clone IMAGE: |
| | | | | 5535936, mRNA |
| Data Set 1 | 250 gene model | 205429_s_at | MPP6 | membrane protein, palmitoylated 6 (MAGUK p55 subfamily member 6) |
| Data Set 1 | 250 gene model | 202180_s_at | MVP | major vault protein |
| Data Set 1 | 250 gene model | 213982_s_at | RABGAP1L | RAB GTPase activating protein 1-like |
| Data Set 1 | 250 gene model | 211126_s_at | CSRP2 | cysteine and glycine-rich protein 2 |
| Data Set 1 | 250 gene model | 205132_at | ACTC | actin, alpha, cardiac muscle |
| Data Set 1 | 250 gene model | 213071_at | DPT | dermatopontin |
| Data Set 1 | 250 gene model | 208430_s_at | DTNA | dystrobrevin, alpha |
| Data Set 1 | 250 gene model | 206453_s_at | NDRG2 | NDRG family member 2 |
| Data Set 1 | 250 gene model | 218979_at | C9orf76 | chromosome 9 open reading frame 76 |
| Data Set 1 | 250 gene model | 220751_s_at | C5orf4 | chromosome 5 open reading frame 4 |
| Data Set 1 | 250 gene model | 213564_x_at | LDHB | lactate dehydrogenase B |
| Data Set 1 | 250 gene model | 209651_at | TGFB1I1 | transforming growth factor beta 1 induced transcript 1 |
| Data Set 1 | 250 gene model | 218224_at | PNMA1 | paraneoplastic antigen MA1 |
| Data Set 1 | 250 gene model | 203219_s_at | APRT | adenine phosphoribosyltransferase |
| Data Set 1 | 250 gene model | 201798_s_at | FER1L3 | fer-1-like 3, myoferlin (C. elegans) |
| Data Set 1 | 250 gene model | 201462_at | SCRN1 | secernin 1 |
| Data Set 1 | 250 gene model | 212254_s_at | DST | dystonin |
| Data Set 1 | 250 gene model | 204352_at | TRAF5 | TNF receptor-associated factor 5 |
| Data Set 1 | 250 gene model | 201583_s_at | SEC23B | Sec23 homolog B (S. cerevisiae) |
| Data Set 1 | 250 gene model | 218073_s_at | TMEM48 | transmembrane protein 48 |
| Data Set 1 | 250 gene model | 209934_s_at | ATP2C1 | ATPase, Ca++ transporting, type 2C, member 1 |
| Data Set 1 | 250 gene model | 204099_at | SMARCD3 | SWI/SNF related, matrix associated, actin dependent regulator of |
| | | | | chromatin, subfamily d, member 3 |
| Data Set 1 | 250 gene model | 205128_x_at | PTGS1 | prostaglandin-endoperoxide synthase 1 (prostaglandin G/H synthase |
| | | | | and cyclooxygenase) |
| Data Set 1 | 250 gene model | 219127_at | MGC11242 | hypothetical protein MGC11242 |
| Data Set 1 | 250 gene model | 203281_s_at | UBE1L | ubiquitin-activating enzyme E1-like |
| Data Set 1 | 250 gene model | 203705_s_at | FZD7 | frizzled homolog 7 (Drosophila) |
| Data Set 1 | 250 gene model | 217979_at | TM4SF13 | Tetraspanin 13 |
| Data Set 1 | 250 gene model | 823_at | CX3CL1 | chemokine (C—X3—C motif) ligand 1 |
| Data Set 1 | 250 gene model | 210298_x_at | FHL1 | four and a half LIM domains 1 |
| Data Set 1 | 250 gene model | 208789_at | PTRF | polymerase I and transcript release factor |
| Data Set 1 | 250 gene model | 221016_s_at | TCF7L1 | transcription factor 7-like 1 (T-cell specific, HMG-box) /// |
| | | | | transcription factor 7-like 1 (T-cell specific, HMG-box) |
| Data Set 1 | 250 gene model | 200807_s_at | HSPD1 | heat shock 60 kDa protein 1 (chaperonin) |
| Data Set 1 | 250 gene model | 201900_s_at | AKR1A1 | aldo-keto reductase family 1, member A1 (aldehyde reductase) |
| Data Set 1 | 250 gene model | 202269_x_at | GBP1 | guanylate binding protein 1, interferon-inducible, 67 kDa /// |
| | | | | guanylate binding protein 1, interferon-inducible, 67 kDa |
| Data Set 1 | 250 gene model | 204793_at | GPRASP1 | G protein-coupled receptor associated sorting protein 1 |
| Data Set 1 | 250 gene model | 212187_x_at | PTGDS | prostaglandin D2 synthase 21 kDa (brain) |
| Data Set 1 | 250 gene model | 201923_at | PRDX4 | peroxiredoxin 4 |
| Data Set 1 | 250 gene model | 210751_s_at | RGN | regucalcin (senescence marker protein-30) |
| Data Set 1 | 250 gene model | 209288_s_at | CDC42EP3 | CDC42 effector protein (Rho GTPase binding) 3 |
| Data Set 1 | 250 gene model | 207414_s_at | PCSK6 | proprotein convertase subtilisin/kexin type 6 |
| Data Set 1 | 250 gene model | 204875_s_at | GMDS | GDP-mannose 4,6-dehydratase |
| Data Set 1 | 250 gene model | 219405_at | TRIM68 | tripartite motif-containing 68 |
| Data Set 1 | 250 gene model | 205364_at | ACOX2 | acyl-Coenzyme A oxidase 2, branched chain |
| Data Set 1 | 250 gene model | 214404_x_at | SPDEF | SAM pointed domain containing ets transcription factor |
| Data Set 1 | 250 gene model | 202732_at | PKIG | protein kinase (cAMP-dependent, catalytic) inhibitor gamma |
| Data Set 1 | 250 gene model | 212463_at | CD59 | CD59 antigen p18-20 (antigen identified by monoclonal antibodies |
| | | | | 16.3A5, EJ16, EJ30, EL32 and G344) |
| Data Set 1 | 250 gene model | 217762_s_at | RAB31 | RAB31, member RAS oncogene family |
| Data Set 1 | 250 gene model | 201850_at | CAPG | capping protein (actin filament), gelsolin-like |
| Data Set 1 | 250 gene model | 217763_s_at | RAB31 | RAB31, member RAS oncogene family |
| Data Set 1 | 250 gene model | 213010_at | PRKCDBP | protein kinase C, delta binding protein |
| Data Set 1 | 250 gene model | 219518_s_at | ELL3 | elongation factor RNA polymerase II-like 3 |
| Data Set 1 | 250 gene model | 201689_s_at | TPD52 | tumor protein D52 |
| Data Set 1 | 250 gene model | 214505_s_at | FHL1 | four and a half LIM domains 1 |
| Data Set 1 | 250 gene model | 201601_x_at | IFITM1 | interferon induced transmembrane protein 1 (9-27) |
| Data Set 1 | 250 gene model | 209074_s_at | TU3A | TU3A protein |
| Data Set 1 | 250 gene model | 218427_at | SDCCAG3 | serologically defined colon cancer antigen 3 |
| Data Set 1 | 250 gene model | 204753_s_at | HLF | hepatic leukemia factor |
| Data Set 1 | 250 gene model | 214598_at | CLDN8 | claudin 8 |
| Data Set 1 | 250 gene model | 201631_s_at | IER3 | immediate early response 3 |
| Data Set 1 | 250 gene model | 204400_at | EFS | embryonal Fyn-associated substrate |
| Data Set 1 | 250 gene model | 217771_at | GOLPH2 | golgi phosphoprotein 2 |
| Data Set 1 | 250 gene model | 219152_at | PODXL2 | podocalyxin-like 2 |
| Data Set 1 | 250 gene model | 202454_s_at | ERBB3 | v-erb-b2 erythroblastic leukemia viral oncogene homolog 3 (avian) |
| Data Set 1 | 250 gene model | 214039_s_at | LAPTM4B | lysosomal associated protein transmembrane 4 beta |
| Data Set 1 | 250 gene model | 205303_at | KCNJ8 | potassium inwardly-rectifying channel, subfamily J, member 8 |
| Data Set 1 | 250 gene model | 209583_s_at | CD200 | CD200 antigen |
| Data Set 1 | 250 gene model | 205743_at | STAC | SH3 and cysteine rich domain |
| Data Set 1 | 250 gene model | 204284_at | PPP1R3C | protein phosphatase 1, regulatory (inhibitor) subunit 3C |
| Data Set 1 | 250 gene model | 218611_at | IER5 | immediate early response 5 |
| Data Set 1 | 250 gene model | 207030_s_at | CSRP2 | cysteine and glycine-rich protein 2 |
| Data Set 1 | 250 gene model | 201690_s_at | TPD52 | tumor protein D52 |
| Data Set 1 | 250 gene model | 214091_s_at | GPX3 | glutathione peroxidase 3 (plasma) |
| Data Set 1 | 250 gene model | 211724_x_at | FLJ20323 | hypothetical protein FLJ20323 /// hypothetical protein FLJ20323 |
| Data Set 1 | 250 gene model | 201539_s_at | FHL1 | four and a half LIM domains 1 |
| Data Set 1 | 250 gene model | 201060_x_at | STOM | stomatin |
| Data Set 1 | 250 gene model | 203966_s_at | PPM1A | protein phosphatase 1A (formerly 2C), magnesium-dependent, alpha |
| | | | | isoform /// protein phosphatase 1A (formerly 2C), magnesium-dependent, |
| | | | | alpha isoform |
| Data Set 1 | 250 gene model | 203851_at | IGFBP6 | insulin-like growth factor binding protein 6 |
| Data Set 1 | 250 gene model | 200903_s_at | AHCY | S-adenosylhomocysteine hydrolase |
| Data Set 1 | 250 gene model | 215016_x_at | DST | dystonin |
| Data Set 1 | 250 gene model | 209291_at | ID4 | inhibitor of DNA binding 4, dominant negative helix-loop-helix protein |
| Data Set 1 | 250 gene model | 207480_s_at | MEIS2 | Meis1, myeloid ecotropic viral integration site 1 homolog 2 (mouse) |
| Data Set 1 | 250 gene model | 219856_at | C1orf116 | chromosome 1 open reading frame 116 |
| Data Set 1 | 250 gene model | 201272_at | AKR1B1 | aldo-keto reductase family 1, member B1 (aldose reductase) |
| Data Set 1 | 250 gene model | 216251_s_at | KIAA0153 | KIAA0153 protein |
| Data Set 1 | 250 gene model | 213085_s_at | KIBRA | KIBRA protein |
| Data Set 1 | 250 gene model | 205769_at | SLC27A2 | solute carrier family 27 (fatty acid transporter), member 2 |
| Data Set 1 | 250 gene model | 203423_at | RBP1 | retinol binding protein 1, cellular |
| Data Set 1 | 250 gene model | 203186_s_at | S100A4 | S100 calcium binding protein A4 (calcium protein, calvasculin, |
| | | | | metastasin, murine placental homolog) |
| Data Set 1 | 250 gene model | 212445_s_at | NEDD4L | neural precursor cell expressed, developmentally down-regulated 4-like |
| Data Set 1 | 250 gene model | 220933_s_at | ZCCHC6 | zinc finger, CCHC domain containing 6 |
| Data Set 1 | 250 gene model | 218186_at | RAB25 | RAB25, member RAS oncogene family |
| Data Set 1 | 250 gene model | 212640_at | PTPLB | protein tyrosine phosphatase-like (proline instead of catalytic arginine), |
| | | | | member b |
| Data Set 1 | 250 gene model | 209550_at | NDN | necdin homolog (mouse) |
| Data Set 1 | 250 gene model | 201348_at | GPX3 | glutathione peroxidase 3 (plasma) |
| Data Set 1 | 250 gene model | 207266_x_at | RBMS1 | RNA binding motif, single stranded interacting protein 1 |
| Data Set 1 | 250 gene model | 203397_s_at | GALNT3 | UDP-N-acetyl-alpha-D-galactosamine:polypeptide N-acetylgalactosaminyl |
| | | | | transferase 3 (GalNAc-T3) |
| Data Set 1 | 250 gene model | 218198_at | DHX32 | DEAH (Asp-Glu-Ala-His) box polypeptide 32 |
| Data Set 1 | 250 gene model | 200986_at | SERPING1 | serpin peptidase inhibitor, clade G (C1 inhibitor), member 1 |
| | | | | (angioedema, hereditary) |
| Data Set 1 | 250 gene model | 221582_at | HIST3H2A | histone 3, H2a |
| Data Set 1 | 250 gene model | 204570_at | COX7A1 | cytochrome c oxidase subunit VIIa polypeptide 1 (muscle) |
| Data Set 1 | 250 gene model | 200644_at | MARCKSL1 | MARCKS-like 1 |
| Data Set 1 | 250 gene model | 201667_at | GJA1 | gap junction protein, alpha 1, 43 kDa (connexin 43) |
| Data Set 1 | 250 gene model | 211715_s_at | BDH | 3-hydroxybutyrate dehydrogenase (heart, mitochondrial) /// |
| | | | | 3-hydroxybutyrate dehydrogenase (heart, mitochondrial) |
| Data Set 1 | 250 gene model | 217080_s_at | HOMER2 | homer homolog 2 (Drosophila) |
| Data Set 1 | 250 gene model | 219121_s_at | RBM35A | RNA binding motif protein 35A |
| Data Set 1 | 250 gene model | 218223_s_at | CKIP-1 | CK2 interacting protein 1; HQ0024c protein |
| Data Set 1 | 250 gene model | 213288_at | OACT2 | O-acyltransferase (membrane bound) domain containing 2 |
| Data Set 1 | 250 gene model | 209863_s_at | TP73L | tumor protein p73-like |
| Data Set 1 | 250 gene model | 202005_at | ST14 | suppression of tumorigenicity 14 (colon carcinoma, matriptase, epithin) |
| Data Set 1 | 250 gene model | 203324_s_at | CAV2 | caveolin 2 |
| Data Set 1 | 250 gene model | 205265_s_at | APEG1 | aortic preferentially expressed gene 1 |
| Data Set 1 | 250 gene model | 208747_s_at | C1S | complement component 1, s subcomponent |
| Data Set 1 | 250 gene model | 212647_at | RRAS | related RAS viral (r-ras) oncogene homolog |
| Data Set 1 | 250 gene model | 214156_at | MYRIP | myosin VIIA and Rab interacting protein |
| Data Set 1 | 250 gene model | 203065_s_at | CAV1 | caveolin 1, caveolae protein, 22 kDa |
| Data Set 1 | 250 gene model | 200923_at | LGALS3BP | lectin, galactoside-binding, soluble, 3 binding protein |
| Data Set 1 | 250 gene model | 203748_x_at | RBMS1 | RNA binding motif, single stranded interacting protein 1 |
| Data Set 1 | 250 gene model | 205578_at | ROR2 | receptor tyrosine kinase-like orphan receptor 2 |
| Data Set 1 | 250 gene model | 212430_at | RNPC1 | RNA-binding region (RNP1, RRM) containing 1 /// RNA-binding |
| | | | | region (RNP1, RRM) containing 1 |
| Data Set 1 | 250 gene model | 218980_at | FHOD3 | formin homology 2 domain containing 3 |
| Data Set 1 | 250 gene model | 200895_s_at | FKBP4 | FK506 binding protein 4, 59 kDa |
| Data Set 1 | 250 gene model | 219829_at | ITGB1BP2 | integrin beta 1 binding protein (melusin) 2 |
| Data Set 1 | 250 gene model | 201482_at | QSCN6 | quiescin Q6 |
| Data Set 1 | 250 gene model | 203545_at | ALG8 | asparagine-linked glycosylation 8 homolog (yeast, alpha-1,3-glucosyl- |
| | | | | transferase) |
| Data Set 1 | 250 gene model | 217973_at | DCXR | dicarbonyl/L-xylulose reductase |
| Data Set 1 | 250 gene model | 201315_x_at | IFITM2 | interferon induced transmembrane protein 2 (1-8D) |
| Data Set 1 | 250 gene model | 203706_s_at | FZD7 | frizzled homolog 7 (Drosophila) |
| Data Set 1 | 250 gene model | 221462_x_at | KLK15 | kallikrein 15 |
| Data Set 1 | 250 gene model | 209170_s_at | GPM6B | glycoprotein M6B |
| Data Set 1 | 250 gene model | 204993_at | GNAZ | guanine nucleotide binding protein (G protein), alpha z polypeptide |
| Data Set 1 | 250 gene model | 209114_at | TSPAN1 | tetraspanin 1 |
| Data Set 1 | 250 gene model | 219685_at | TMEM35 | transmembrane protein 35 |
| Data Set 1 | 250 gene model | 209691_s_at | DOK4 | docking protein 4 |
| Data Set 1 | 250 gene model | 212203_x_at | IFITM3 | interferon induced transmembrane protein 3 (1-8U) |
| Data Set 1 | 250 gene model | 205542_at | STEAP1 | six transmembrane epithelial antigen of the prostate 1 |
| Data Set 1 | 250 gene model | 212680_x_at | PPP1R14B | protein phosphatase 1, regulatory (inhibitor) subunit 14B |
| Data Set 1 | 250 gene model | 1598_g_at | GAS6 | growth arrest-specific 6 |
| Data Set 1 | 250 gene model | 209340_at | UAP1 | UDP-N-acteylglucosamine pyrophosphorylase 1 |
| Data Set 1 | 250 gene model | 208131_s_at | PTGIS | prostaglandin I2 (prostacyclin) synthase /// prostaglandin I2 (prostacyclin) |
| | | | | synthase |
| Data Set 1 | 250 gene model | 213004_at | ANGPTL2 | angiopoietin-like 2 |
| Data Set 1 | 250 gene model | 203892_at | WFDC2 | WAP four-disulfide core domain 2 |
| Data Set 1 | 250 gene model | 203911_at | RAP1GA1 | RAP1, GTPase activating protein 1 |
| Data Set 1 | 250 gene model | 206860_s_at | FLJ20323 | hypothetical protein FLJ20323 |
| Data Set 1 | 250 gene model | 209696_at | FBP1 | fructose-1,6-bisphosphatase 1 |
| Data Set 1 | 250 gene model | 210547_x_at | ICA1 | islet cell autoantigen 1, 69 kDa |
| Data Set 1 | 250 gene model | 204734_at | KRT15 | keratin 15 |
| Data Set 1 | 250 gene model | 203638_s_at | FGFR2 | fibroblast growth factor receptor 2 (bacteria-expressed kinase, keratinocyte |
| | | | | growth factor receptor, craniofacial dysostosis 1, Crouzon syndrome, |
| | | | | Pfeiffer syndrome, Jackson-Weiss syndrome) |
| Data Set 1 | 250 gene model | 200971_s_at | SERP1 | stress-associated endoplasmic reticulum protein 1 |
| Data Set 1 | 250 gene model | 216565_x_at | LOC391020 | similar to Interferon-induced transmembrane protein 3 (Interferon-inducible |
| | | | | protein 1-8U) |
| Data Set 1 | 250 gene model | 209434_s_at | PPAT | phosphoribosyl pyrophosphate amidotransferase |
| Data Set 1 | 250 gene model | 209804_at | DCLRE1A | DNA cross-link repair 1A (PSO2 homolog, S. cerevisiae) |
| Data Set 1 | 250 gene model | 202893_at | UNC13B | unc-13 homolog B (C. elegans) |
| Data Set 1 | 250 gene model | 218313_s_at | GALNT7 | UDP-N-acetyl-alpha-D-galactosamine:polypeptide N-acetyl- |
| | | | | galactosaminyltransferase 7 (GalNAc-T7) |
| Data Set 2 | 5 gene model | 200982_s_at | ANXA6 | annexin A6 |
| Data Set 2 | 5 gene model | 205304_s_at | KCNJ8 | potassium inwardly-rectifying channel, subfamily J, member 8 |
| Data Set 2 | 5 gene model | 227554_at | LOC402560 | Hypothetical LOC402560 |
| Data Set 2 | 5 gene model | 235867_at | GSTM3 | glutathione S-transferase M3 (brain) |
| Data Set 2 | 5 gene model | 213556_at | LOC390940 | similar to R28379_1 |
| Data Set 2 | 10 gene model | 213924_at | MPPE1 | Metallophosphoesterase 1 |
| Data Set 2 | 10 gene model | 205303_at | KCNJ8 | potassium inwardly-rectifying channel, subfamily J, member 8 |
| Data Set 2 | 10 gene model | 208792_s_at | CLU | clusterin |
| Data Set 2 | 10 gene model | 230087_at | PRIMA1 | proline rich membrane anchor 1 |
| Data Set 2 | 10 gene model | 218094_s_at | DBNDD2 | dysbindin (dystrobrevin binding protein 1) domain containing 2 |
| Data Set 2 | 10 gene model | 205304_s_at | KCNJ8 | potassium inwardly-rectifying channel, subfamily J, member 8 |
| Data Set 2 | 10 gene model | 1553102_a_at | CCDC69 | coiled-coil domain containing 69 |
| Data Set 2 | 10 gene model | 227554_at | LOC402560 | Hypothetical LOC402560 |
| Data Set 2 | 10 gene model | 209434_s_at | PPAT | phosphoribosyl pyrophosphate amidotransferase |
| Data Set 2 | 10 gene model | 231118_at | ANKRD35 | ankyrin repeat domain 35 |
| Data Set 2 | 20 gene model | 201798_s_at | FER1L3 | fer-1-like 3, myoferlin (C. elegans) |
| Data Set 2 | 20 gene model | 222043_at | CLU | clusterin |
| Data Set 2 | 20 gene model | 219670_at | C1orf165 | chromosome 1 open reading frame 165 |
| Data Set 2 | 20 gene model | 223843_at | SCARA3 | scavenger receptor class A, member 3 |
| Data Set 2 | 20 gene model | 203323_at | CAV2 | caveolin 2 |
| Data Set 2 | 20 gene model | 230067_at | FLJ30707 | Hypothetical protein FLJ30707 |
| Data Set 2 | 20 gene model | 212736_at | C16orf45 | chromosome 16 open reading frame 45 |
| Data Set 2 | 20 gene model | 221898_at | PDPN | podoplanin |
| Data Set 2 | 20 gene model | 205577_at | PYGM | phosphorylase, glycogen; muscle (McArdle syndrome, glycogen |
| | | | | storage disease type V) |
| Data Set 2 | 20 gene model | 204099_at | SMARCD3 | SWI/SNF related, matrix associated, actin dependent regulator of |
| | | | | chromatin, subfamily d, member 3 |
| Data Set 2 | 20 gene model | 224710_at | RAB34 | RAB34, member RAS oncogene family |
| Data Set 2 | 20 gene model | 203151_at | MAP1A | microtubule-associated protein 1A |
| Data Set 2 | 20 gene model | 201590_x_at | ANXA2 | annexin A2 |
| Data Set 2 | 20 gene model | 210427_x_at | ANXA2 | annexin A2 |
| Data Set 2 | 20 gene model | 218421_at | CERK | ceramide kinase |
| Data Set 2 | 20 gene model | 209356_x_at | EFEMP2 | EGF-containing fibulin-like extracellular matrix protein 2 |
| Data Set 2 | 20 gene model | 208792_s_at | CLU | clusterin |
| Data Set 2 | 20 gene model | 219525_at | FLJ10847 | hypothetical protein FLJ10847 |
| Data Set 2 | 20 gene model | 204777_s_at | MAL | mal, T-cell differentiation protein |
| Data Set 2 | 20 gene model | 213503_x_at | ANXA2 | annexin A2 |
| Data Set 2 | 50 gene model | 1552701_a_at | COP1 | caspase-1 dominant-negative inhibitor pseudo-ICE |
| Data Set 2 | 50 gene model | 204115_at | GNG11 | guanine nucleotide binding protein (G protein), gamma 11 |
| Data Set 2 | 50 gene model | 244111_at | KA21 | truncated type I keratin KA21 |
| Data Set 2 | 50 gene model | 220751_s_at | C5orf4 | chromosome 5 open reading frame 4 |
| Data Set 2 | 50 gene model | 244050_at | PTPLAD2 | protein tyrosine phosphatase-like A domain containing 2 |
| Data Set 2 | 50 gene model | 214027_x_at | DES /// FAM48A | desmin /// family with sequence similarity 48, member A |
| Data Set 2 | 50 gene model | 222744_s_at | TMLHE | trimethyllysine hydroxylase, epsilon |
| Data Set 2 | 50 gene model | 1553995_a_at | NT5E | 5′-nucleotidase, ecto (CD73) |
| Data Set 2 | 50 gene model | 208791_at | CLU | clusterin |
| Data Set 2 | 50 gene model | 201136_at | PLP2 | proteolipid protein 2 (colonic epithelium-enriched) |
| Data Set 2 | 50 gene model | 226047_at | MRVI1 | Murine retrovirus integration site 1 homolog |
| Data Set 2 | 50 gene model | 236383_at | — | Transcribed locus |
| Data Set 2 | 50 gene model | 211562_s_at | LMOD1 | leiomodin 1 (smooth muscle) |
| Data Set 2 | 50 gene model | 222669_s_at | SBDS | Shwachman-Bodian-Diamond syndrome |
| Data Set 2 | 50 gene model | 207030_s_at | CSRP2 | cysteine and glycine-rich protein 2 |
| Data Set 2 | 50 gene model | 204735_at | PDE4A | phosphodiesterase 4A, cAMP-specific (phosphodiesterase E2 |
| | | | | dunce homolog, Drosophila) |
| Data Set 2 | 50 gene model | 218864_at | TNS1 | tensin 1 |
| Data Set 2 | 50 gene model | 214369_s_at | RASGRP2 | RAS guanyl releasing protein 2 (calcium and DAG-regulated) |
| Data Set 2 | 50 gene model | 205578_at | ROR2 | receptor tyrosine kinase-like orphan receptor 2 |
| Data Set 2 | 50 gene model | 204099_at | SMARCD3 | SWI/SNF related, matrix associated, actin dependent regulator of |
| | | | | chromatin, subfamily d, member 3 |
| Data Set 2 | 50 gene model | 213309_at | PLCL2 | phospholipase C-like 2 |
| Data Set 2 | 50 gene model | 207836_s_at | RBPMS | RNA binding protein with multiple splicing |
| Data Set 2 | 50 gene model | 203921_at | CHST2 | carbohydrate (N-acetylglucosamine-6-O) sulfotransferase 2 |
| Data Set 2 | 50 gene model | 203951_at | CNN1 | calponin 1, basic, smooth muscle |
| Data Set 2 | 50 gene model | 217111_at | AMACR | alpha-methylacyl-CoA racemase |
| Data Set 2 | 50 gene model | 210869_s_at | MCAM | melanoma cell adhesion molecule |
| Data Set 2 | 50 gene model | 226926_at | ZD52F10 | dermokine |
| Data Set 2 | 50 gene model | 220034_at | IRAK3 | interleukin-1 receptor-associated kinase 3 |
| Data Set 2 | 50 gene model | 238151_at | TUBB6 | Tubulin, beta 6 |
| Data Set 2 | 50 gene model | 201842_s_at | EFEMP1 | EGF-containing fibulin-like extracellular matrix protein 1 |
| Data Set 2 | 50 gene model | 209651_at | TGFB1I1 | transforming growth factor beta 1 induced transcript 1 |
| Data Set 2 | 50 gene model | 203632_s_at | GPRC5B | G protein-coupled receptor, family C, group 5, member B |
| Data Set 2 | 50 gene model | 49452_at | ACACB | acetyl-Coenzyme A carboxylase beta |
| Data Set 2 | 50 gene model | 203766_s_at | LMOD1 | leiomodin 1 (smooth muscle) |
| Data Set 2 | 50 gene model | 225381_at | LOC399959 | hypothetical gene supported by BX647608 |
| Data Set 2 | 50 gene model | 209948_at | KCNMB1 | potassium large conductance calcium-activated channel, subfamily |
| | | | | M, beta member 1 |
| Data Set 2 | 50 gene model | 235657_at | — | Transcribed locus |
| Data Set 2 | 50 gene model | 213426_s_at | CAV2 | caveolin 2 |
| Data Set 2 | 50 gene model | 205088_at | CXorf6 | chromosome X open reading frame 6 |
| Data Set 2 | 50 gene model | 227006_at | PPP1R14A | protein phosphatase 1, regulatory (inhibitor) subunit 14A |
| Data Set 2 | 50 gene model | 211276_at | TCEAL2 | transcription elongation factor A (SII)-like 2 |
| Data Set 2 | 50 gene model | 221016_s_at | TCF7L1 | transcription factor 7-like 1 (T-cell specific, HMG-box) /// transcription |
| | | | | factor 7-like 1 (T-cell specific, HMG-box) |
| Data Set 2 | 50 gene model | 207390_s_at | SMTN | smoothelin |
| Data Set 2 | 50 gene model | 211340_s_at | MCAM | melanoma cell adhesion molecule |
| Data Set 2 | 50 gene model | 228080_at | LAYN | layilin |
| Data Set 2 | 50 gene model | 214767_s_at | HSPB6 | heat shock protein, alpha-crystallin-related, B6 |
| Data Set 2 | 50 gene model | 242170_at | ZNF154 | Zinc finger protein 154 (pHZ-92) |
| Data Set 2 | 50 gene model | 205577_at | PYGM | phosphorylase, glycogen; muscle (McArdle syndrome, glycogen |
| | | | | storage disease type V) |
| Data Set 2 | 50 gene model | 230519_at | FLJ30707 | hypothetical protein FLJ30707 |
| Data Set 2 | 50 gene model | 222043_at | CLU | clusterin |
| Data Set 2 | 100 gene model | 203892_at | WFDC2 | WAP four-disulfide core domain 2 |
| Data Set 2 | 100 gene model | 239911_at | — | Full-length cDNA clone CS0DJ013YP06 of T cells (Jurkat cell line) |
| | | | | Cot 10-normalized of Homo sapiens (human) |
| Data Set 2 | 100 gene model | 216548_x_at | HMG4L | high-mobility group (nonhistone chromosomal) protein 4-like |
| Data Set 2 | 100 gene model | 207016_s_at | ALDH1A2 | aldehyde dehydrogenase 1 family, member A2 |
| Data Set 2 | 100 gene model | 210224_at | MR1 | major histocompatibility complex, class I-related |
| Data Set 2 | 100 gene model | 226638_at | ARHGAP23 | Rho GTPase activating protein 23 |
| Data Set 2 | 100 gene model | 214369_s_at | RASGRP2 | RAS guanyl releasing protein 2 (calcium and DAG-regulated) |
| Data Set 2 | 100 gene model | 227188_at | C21orf63 | chromosome 21 open reading frame 63 |
| Data Set 2 | 100 gene model | 205478_at | PPP1R1A | protein phosphatase 1, regulatory (inhibitor) subunit 1A |
| Data Set 2 | 100 gene model | 202949_s_at | FHL2 | four and a half LIM domains 2 |
| Data Set 2 | 100 gene model | 235593_at | ZFHX1B | zinc finger homeobox 1b |
| Data Set 2 | 100 gene model | 228202_at | PLN | Phospholamban |
| Data Set 2 | 100 gene model | 204940_at | PLN | phospholamban |
| Data Set 2 | 100 gene model | 206030_at | ASPA | aspartoacylase (Canavan disease) |
| Data Set 2 | 100 gene model | 212358_at | CLIPR-59 | CLIP-170-related protein |
| Data Set 2 | 100 gene model | 227862_at | LOC388610 | hypothetical LOC388610 |
| Data Set 2 | 100 gene model | 227236_at | TSPAN2 | tetraspanin 2 |
| Data Set 2 | 100 gene model | 225288_at | — | Full-length cDNA clone CS0DI001YP15 of Placenta Cot 25-normalized |
| | | | | of Homo sapiens (human) |
| Data Set 2 | 100 gene model | 218691_s_at | PDLIM4 | PDZ and LIM domain 4 |
| Data Set 2 | 100 gene model | 1552703_s_at | CASP1 /// COP1 | caspase 1, apoptosis-related cysteine peptidase (interleukin 1, beta, |
| | | | | convertase) /// caspase-1 dominant-negative inhibitor pseudo-ICE |
| Data Set 2 | 100 gene model | 231292_at | EID3 | E1A-like inhibitor of differentiation 3 |
| Data Set 2 | 100 gene model | 210102_at | LOH11CR2A | loss of heterozygosity, 11, chromosomal region 2, gene A |
| Data Set 2 | 100 gene model | 206355_at | GNAL | guanine nucleotide binding protein (G protein), alpha activating |
| | | | | activity polypeptide, olfactory type |
| Data Set 2 | 100 gene model | 227742_at | CLIC6 | chloride intracellular channel 6 |
| Data Set 2 | 100 gene model | 231202_at | ALDH1L2 | aldehyde dehydrogenase 1 family, member L2 |
| Data Set 2 | 100 gene model | 205132_at | ACTC | actin, alpha, cardiac muscle |
| Data Set 2 | 100 gene model | 209087_x_at | MCAM | melanoma cell adhesion molecule |
| Data Set 2 | 100 gene model | 236936_at | — | — |
| Data Set 2 | 100 gene model | 211126_s_at | CSRP2 | cysteine and glycine-rich protein 2 |
| Data Set 2 | 100 gene model | 202794_at | INPP1 | inositol polyphosphate-1-phosphatase |
| Data Set 2 | 100 gene model | 241803_s_at | — | — |
| Data Set 2 | 100 gene model | 204037_at | EDG2 /// | endothelial differentiation, lysophosphatidic acid G-protein-coupled |
| | | | LOC644923 | receptor, 2 /// hypothetical protein LOC644923 |
| Data Set 2 | 100 gene model | 204993_at | GNAZ | guanine nucleotide binding protein (G protein), alpha z polypeptide |
| Data Set 2 | 100 gene model | 1555630_a_at | RAB34 | RAB34, member RAS oncogene family |
| Data Set 2 | 100 gene model | 209789_at | CORO2B | coronin, actin binding protein, 2B |
| Data Set 2 | 100 gene model | 244167_at | SERGEF | Secretion regulating guanine nucleotide exchange factor |
| Data Set 2 | 100 gene model | 203851_at | IGFBP6 | insulin-like growth factor binding protein 6 |
| Data Set 2 | 100 gene model | 229648_at | — | Transcribed locus |
| Data Set 2 | 100 gene model | 202196_s_at | DKK3 | dickkopf homolog 3 (Xenopus laevis) |
| Data Set 2 | 100 gene model | 226303_at | PGM5 | phosphoglucomutase 5 |
| Data Set 2 | 100 gene model | 201431_s_at | DPYSL3 | dihydropyrimidinase-like 3 |
| Data Set 2 | 100 gene model | 213746_s_at | FLNA | filamin A, alpha (actin binding protein 280) |
| Data Set 2 | 100 gene model | 212091_s_at | COL6A1 | collagen, type VI, alpha 1 |
| Data Set 2 | 100 gene model | 1569956_at | — | Homo sapiens, clone IMAGE: 4413783, mRNA |
| Data Set 2 | 100 gene model | 203650_at | PROCR | protein C receptor, endothelial (EPCR) |
| Data Set 2 | 100 gene model | 204310_s_at | NPR2 | natriuretic peptide receptor B/guanylate cyclase B (atrionatriuretic |
| | | | | peptide receptor B) |
| Data Set 2 | 100 gene model | 222669_s_at | SBDS | Shwachman-Bodian-Diamond syndrome |
| Data Set 2 | 100 gene model | 205578_at | ROR2 | receptor tyrosine kinase-like orphan receptor 2 |
| Data Set 2 | 100 gene model | 212813_at | JAM3 | junctional adhesion molecule 3 |
| Data Set 2 | 100 gene model | 230271_at | — | Homo sapiens, clone IMAGE: 4512785, mRNA |
| Data Set 2 | 100 gene model | 236383_at | — | Transcribed locus |
| Data Set 2 | 100 gene model | 210880_s_at | EFS | embryonal Fyn-associated substrate |
| Data Set 2 | 100 gene model | 206813_at | CTF1 | cardiotrophin 1 |
| Data Set 2 | 100 gene model | 45297_at | EHD2 | EH-domain containing 2 |
| Data Set 2 | 100 gene model | 200621_at | CSRP1 | cysteine and glycine-rich protein 1 |
| Data Set 2 | 100 gene model | 226280_at | — | CDNA FLJ43545 fis, clone PROST2011631 |
| Data Set 2 | 100 gene model | 213170_at | GPX7 | glutathione peroxidase 7 |
| Data Set 2 | 100 gene model | 1552785_at | FLJ37549 | hypothetical protein FLJ37549 |
| Data Set 2 | 100 gene model | 203370_s_at | PDLIM7 | PDZ and LIM domain 7 (enigma) |
| Data Set 2 | 100 gene model | 223842_s_at | SCARA3 | scavenger receptor class A, member 3 |
| Data Set 2 | 100 gene model | 206465_at | ACSBG1 | acyl-CoA synthetase bubblegum family member 1 |
| Data Set 2 | 100 gene model | 201136_at | PLP2 | proteolipid protein 2 (colonic epithelium-enriched) |
| Data Set 2 | 100 gene model | 43427_at | ACACB | acetyl-Coenzyme A carboxylase beta |
| Data Set 2 | 100 gene model | 204735_at | PDE4A | phosphodiesterase 4A, cAMP-specific (phosphodiesterase E2 |
| | | | | dunce homolog, Drosophila) |
| Data Set 2 | 100 gene model | 213010_at | PRKCDBP | protein kinase C, delta binding protein |
| Data Set 2 | 100 gene model | 223095_at | MARVELD1 | MARVEL domain containing 1 |
| Data Set 2 | 100 gene model | 226304_at | HSPB6 | heat shock protein, alpha-crystallin-related, B6 |
| Data Set 2 | 100 gene model | 243209_at | KCNQ4 | potassium voltage-gated channel, KQT-like subfamily, member 4 |
| Data Set 2 | 100 gene model | 244111_at | KA21 | truncated type I keratin KA21 |
| Data Set 2 | 100 gene model | 1552701_a_at | COP1 | caspase-1 dominant-negative inhibitor pseudo-ICE |
| Data Set 2 | 100 gene model | 207836_s_at | RBPMS | RNA binding protein with multiple splicing |
| Data Set 2 | 100 gene model | 211564_s_at | PDLIM4 | PDZ and LIM domain 4 |
| Data Set 2 | 100 gene model | 208690_s_at | PDLIM1 | PDZ and LIM domain 1 (elfin) |
| Data Set 2 | 100 gene model | 207030_s_at | CSRP2 | cysteine and glycine-rich protein 2 |
| Data Set 2 | 100 gene model | 217111_at | AMACR | alpha-methylacyl-CoA racemase |
| Data Set 2 | 100 gene model | 214027_x_at | DES /// FAM48A | desmin /// family with sequence similarity 48, member A |
| Data Set 2 | 100 gene model | 211562_s_at | LMOD1 | leiomodin 1 (smooth muscle) |
| Data Set 2 | 100 gene model | 244050_at | PTPLAD2 | protein tyrosine phosphatase-like A domain containing 2 |
| Data Set 2 | 100 gene model | 1553995_a_at | NT5E | 5′-nucleotidase, ecto (CD73) |
| Data Set 2 | 100 gene model | 204069_at | MEIS1 | Meis1, myeloid ecotropic viral integration site 1 homolog (mouse) |
| Data Set 2 | 100 gene model | 206122_at | SOX15 | SRY (sex determining region Y)-box 15 |
| Data Set 2 | 100 gene model | 210869_s_at | MCAM | melanoma cell adhesion molecule |
| Data Set 2 | 100 gene model | 204115_at | GNG11 | guanine nucleotide binding protein (G protein), gamma 11 |
| Data Set 2 | 100 gene model | 225381_at | LOC399959 | hypothetical gene supported by BX647608 |
| Data Set 2 | 100 gene model | 226926_at | ZD52F10 | dermokine |
| Data Set 2 | 100 gene model | 204099_at | SMARCD3 | SWI/SNF related, matrix associated, actin dependent regulator of |
| | | | | chromatin, subfamily d, member 3 |
| Data Set 2 | 100 gene model | 205088_at | CXorf6 | chromosome X open reading frame 6 |
| Data Set 2 | 100 gene model | 203632_s_at | GPRC5B | G protein-coupled receptor, family C, group 5, member B |
| Data Set 2 | 100 gene model | 203921_at | CHST2 | carbohydrate (N-acetylglucosamine-6-O) sulfotransferase 2 |
| Data Set 2 | 100 gene model | 228080_at | LAYN | layilin |
| Data Set 2 | 100 gene model | 218864_at | TNS1 | tensin 1 |
| Data Set 2 | 100 gene model | 203951_at | CNN1 | calponin 1, basic, smooth muscle |
| Data Set 2 | 100 gene model | 220751_s_at | C5orf4 | chromosome 5 open reading frame 4 |
| Data Set 2 | 100 gene model | 208791_at | CLU | clusterin |
| Data Set 2 | 100 gene model | 212886_at | CCDC69 | coiled-coil domain containing 69 |
| Data Set 2 | 100 gene model | 229480_at | LOC402560 | hypothetical LOC402560 |
| Data Set 2 | 100 gene model | 209434_s_at | PPAT | phosphoribosyl pyrophosphate amidotransferase |
| Data Set 2 | 100 gene model | 213556_at | LOC390940 | similar to R28379_1 |
| Data Set 2 | 100 gene model | 231118_at | ANKRD35 | ankyrin repeat domain 35 |
| Data Set 2 | 100 gene model | 205083_at | AOX1 | aldehyde oxidase 1 |
| Data Set 2 | 250 gene model | 202274_at | ACTG2 | actin, gamma 2, smooth muscle, enteric |
| Data Set 2 | 250 gene model | 213290_at | COL6A2 | collagen, type VI, alpha 2 |
| Data Set 2 | 250 gene model | 210139_s_at | PMP22 | peripheral myelin protein 22 |
| Data Set 2 | 250 gene model | 229127_at | ATP5J | ATP synthase, H+ transporting, mitochondrial F0 complex, subunit F6 |
| Data Set 2 | 250 gene model | 209427_at | SMTN | smoothelin |
| Data Set 2 | 250 gene model | 223786_at | CHST6 | carbohydrate (N-acetylglucosamine 6-O) sulfotransferase 6 |
| Data Set 2 | 250 gene model | 206600_s_at | SLC16A5 | solute carrier family 16 (monocarboxylic acid transporters), member 5 |
| Data Set 2 | 250 gene model | 219213_at | JAM2 | junctional adhesion molecule 2 |
| Data Set 2 | 250 gene model | 206580_s_at | EFEMP2 | EGF-containing fibulin-like extracellular matrix protein 2 |
| Data Set 2 | 250 gene model | 228141_at | LOC493869 | Similar to RIKEN cDNA 2310016C16 |
| Data Set 2 | 250 gene model | 227862_at | LOC388610 | hypothetical LOC388610 |
| Data Set 2 | 250 gene model | 204570_at | COX7A1 | cytochrome c oxidase subunit VIIa polypeptide 1 (muscle) |
| Data Set 2 | 250 gene model | 227998_at | S100A16 | S100 calcium binding protein A16 |
| Data Set 2 | 250 gene model | 228726_at | — | — |
| Data Set 2 | 250 gene model | 213106_at | — | — |
| Data Set 2 | 250 gene model | 205392_s_at | CCL14 /// CCL15 | chemokine (C-C motif) ligand 14 /// chemokine (C-C motif) ligand 15 |
| Data Set 2 | 250 gene model | 238657_at | UBXD3 | UBX domain containing 3 |
| Data Set 2 | 250 gene model | 216594_x_at | AKR1C1 | aldo-keto reductase family 1, member C1 (dihydrodiol dehydrogenase 1; |
| | | | | 20-alpha (3-alpha)-hydroxysteroid dehydrogenase) |
| Data Set 2 | 250 gene model | 212647_at | RRAS | related RAS viral (r-ras) oncogene homolog |
| Data Set 2 | 250 gene model | 230264_s_at | AP1S2 | adaptor-related protein complex 1, sigma 2 subunit |
| Data Set 2 | 250 gene model | 210619_s_at | HYAL1 | hyaluronoglucosaminidase 1 |
| Data Set 2 | 250 gene model | 224724_at | SULF2 | sulfatase 2 |
| Data Set 2 | 250 gene model | 225242_s_at | CCDC80 | coiled-coil domain containing 80 |
| Data Set 2 | 250 gene model | 218454_at | FLJ22662 | hypothetical protein FLJ22662 |
| Data Set 2 | 250 gene model | 220933_s_at | ZCCHC6 | zinc finger, CCHC domain containing 6 |
| Data Set 2 | 250 gene model | 230933_at | — | Transcribed locus |
| Data Set 2 | 250 gene model | 218423_x_at | VPS54 | vacuolar protein sorting 54 (S. cerevisiae) |
| Data Set 2 | 250 gene model | 218660_at | DYSF | dysferlin, limb girdle muscular dystrophy 2B (autosomal recessive) |
| Data Set 2 | 250 gene model | 213139_at | SNAI2 | snail homolog 2 (Drosophila) |
| Data Set 2 | 250 gene model | 228494_at | PPP1R9A | protein phosphatase 1, regulatory (inhibitor) subunit 9A |
| Data Set 2 | 250 gene model | 201300_s_at | PRNP | prion protein (p27-30) (Creutzfeldt-Jakob disease, Gerstmann-Strausler- |
| | | | | Scheinker syndrome, fatal familial insomnia) |
| Data Set 2 | 250 gene model | 214212_x_at | PLEKHC1 | pleckstrin homology domain containing, family C (with FERM domain) |
| | | | | member 1 |
| Data Set 2 | 250 gene model | 200795_at | SPARCL1 | SPARC-like 1 (mast9, hevin) |
| Data Set 2 | 250 gene model | 1556696_s_at | FLJ42709 | Hypothetical gene supported by AK124699 |
| Data Set 2 | 250 gene model | 200859_x_at | FLNA | filamin A, alpha (actin binding protein 280) |
| Data Set 2 | 250 gene model | 207480_s_at | MEIS2 | Meis1, myeloid ecotropic viral integration site 1 homolog 2 (mouse) |
| Data Set 2 | 250 gene model | 202222_s_at | DES | desmin |
| Data Set 2 | 250 gene model | 201060_x_at | STOM | stomatin |
| Data Set 2 | 250 gene model | 220795_s_at | KIAA1446 | likely ortholog of rat brain-enriched guanylate kinase-associated protein |
| Data Set 2 | 250 gene model | 212097_at | CAV1 | caveolin 1, caveolae protein, 22 kDa |
| Data Set 2 | 250 gene model | 227826_s_at | SORBS2 | Sorbin and SH3 domain containing 2 |
| Data Set 2 | 250 gene model | 1555127_at | MOCS1 | molybdenum cofactor synthesis 1 |
| Data Set 2 | 250 gene model | 212793_at | DAAM2 | dishevelled associated activator of morphogenesis 2 |
| Data Set 2 | 250 gene model | 213001_at | ANGPTL2 | angiopoietin-like 2 |
| Data Set 2 | 250 gene model | 205560_at | PCSK5 | proprotein convertase subtilisin/kexin type 5 |
| Data Set 2 | 250 gene model | 201234_at | ILK | integrin-linked kinase |
| Data Set 2 | 250 gene model | 227899_at | VIT | vitrin |
| Data Set 2 | 250 gene model | 234015_at | NAALADL2 | N-acetylated alpha-linked acidic dipeptidase-like 2 |
| Data Set 2 | 250 gene model | 227066_at | MOBKL2C | MOB1, Mps One Binder kinase activator-like 2C (yeast) |
| Data Set 2 | 250 gene model | 209118_s_at | TUBA3 | tubulin, alpha 3 |
| Data Set 2 | 250 gene model | 202422_s_at | ACSL4 | acyl-CoA synthetase long-chain family member 4 |
| Data Set 2 | 250 gene model | 242874_at | C14orf161 | Chromosome 14 open reading frame 161 |
| Data Set 2 | 250 gene model | 236270_at | NFATC4 | nuclear factor of activated T-cells, cytoplasmic, calcineurin-dependent 4 |
| Data Set 2 | 250 gene model | 221748_s_at | TNS1 | tensin 1 /// tensin 1 |
| Data Set 2 | 250 gene model | 204793_at | GPRASP1 | G protein-coupled receptor associated sorting protein 1 |
| Data Set 2 | 250 gene model | 238115_at | DNAJC18 | DnaJ (Hsp40) homolog, subfamily C, member 18 |
| Data Set 2 | 250 gene model | 220911_s_at | KIAA1305 | KIAA1305 |
| Data Set 2 | 250 gene model | 227233_at | TSPAN2 | tetraspanin 2 |
| Data Set 2 | 250 gene model | 227565_at | — | Transcribed locus |
| Data Set 2 | 250 gene model | 229014_at | FLJ42709 | hypothetical gene supported by AK124699 |
| Data Set 2 | 250 gene model | 201425_at | ALDH2 | aldehyde dehydrogenase 2 family (mitochondrial) |
| Data Set 2 | 250 gene model | 226225_at | MCC | mutated in colorectal cancers |
| Data Set 2 | 250 gene model | 242086_at | SPATA6 | Spermatogenesis associated 6 |
| Data Set 2 | 250 gene model | 239183_at | ANGPTL1 | angiopoietin-like 1 |
| Data Set 2 | 250 gene model | 1568868_at | FLJ16008 | FLJ16008 protein |
| Data Set 2 | 250 gene model | 202148_s_at | PYCR1 | pyrroline-5-carboxylate reductase 1 |
| Data Set 2 | 250 gene model | 204030_s_at | SCHIP1 | schwannomin interacting protein 1 |
| Data Set 2 | 250 gene model | 214066_x_at | NPR2 | natriuretic peptide receptor B/guanylate cyclase B (atrionatriuretic |
| | | | | peptide receptor B) |
| Data Set 2 | 250 gene model | 221436_s_at | CDCA3 | cell division cycle associated 3 /// cell division cycle associated 3 |
| Data Set 2 | 250 gene model | 209685_s_at | PRKCB1 | protein kinase C, beta 1 |
| Data Set 2 | 250 gene model | 227486_at | NT5E | 5′-nucleotidase, ecto (CD73) |
| Data Set 2 | 250 gene model | 1559477_s_at | MEIS1 | Meis1, myeloid ecotropic viral integration site 1 homolog (mouse) |
| Data Set 2 | 250 gene model | 217220_at | — | — |
| Data Set 2 | 250 gene model | 232276_at | HS6ST3 | heparan sulfate 6-O-sulfotransferase 3 |
| Data Set 2 | 250 gene model | 58916_at | KCTD14 | potassium channel tetramerisation domain containing 14 |
| Data Set 2 | 250 gene model | 238463_at | — | Homo sapiens, clone IMAGE: 5309572, mRNA |
| Data Set 2 | 250 gene model | 220974_x_at | SFXN3 | sideroflexin 3 /// sideroflexin 3 |
| Data Set 2 | 250 gene model | 209735_at | ABCG2 | ATP-binding cassette, sub-family G (WHITE), member 2 |
| Data Set 2 | 250 gene model | 228113_at | RAB37 | RAB37, member RAS oncogene family |
| Data Set 2 | 250 gene model | 223395_at | ABI3BP | ABI gene family, member 3 (NESH) binding protein |
| Data Set 2 | 250 gene model | 235897_at | COPZ2 | coatomer protein complex, subunit zeta 2 |
| Data Set 2 | 250 gene model | 241310_at | — | Transcribed locus |
| Data Set 2 | 250 gene model | 202409_at | C11orf43 | chromosome 11 open reading frame 43 |
| Data Set 2 | 250 gene model | 210632_s_at | SGCA | sarcoglycan, alpha (50 kDa dystrophin-associated glycoprotein) |
| Data Set 2 | 250 gene model | 204879_at | PDPN | podoplanin |
| Data Set 2 | 250 gene model | 213068_at | DPT | dermatopontin |
| Data Set 2 | 250 gene model | 211682_x_at | UGT2B28 | UDP glucuronosyltransferase 2 family, polypeptide B28 /// UDP |
| | | | | glucuronosyltransferase 2 family, polypeptide B28 |
| Data Set 2 | 250 gene model | 205547_s_at | TAGLN | transgelin |
| Data Set 2 | 250 gene model | 220113_x_at | POLR1B | polymerase (RNA) I polypeptide B, 128 kDa |
| Data Set 2 | 250 gene model | 57588_at | SLC24A3 | solute carrier family 24 (sodium/potassium/calcium exchanger), member 3 |
| Data Set 2 | 250 gene model | 1554206_at | TMLHE | trimethyllysine hydroxylase, epsilon |
| Data Set 2 | 250 gene model | 204688_at | SGCE | sarcoglycan, epsilon |
| Data Set 2 | 250 gene model | 228584_at | SGCB | sarcoglycan, beta (43 kDa dystrophin-associated glycoprotein) |
| Data Set 2 | 250 gene model | 203510_at | MET | met proto-oncogene (hepatocyte growth factor receptor) |
| Data Set 2 | 250 gene model | 226955_at | FLJ36748 | hypothetical protein FLJ36748 |
| Data Set 2 | 250 gene model | 208335_s_at | DARC | Duffy blood group, chemokine receptor |
| Data Set 2 | 250 gene model | 204418_x_at | GSTM2 | glutathione S-transferase M2 (muscle) |
| Data Set 2 | 250 gene model | 220541_at | MMP26 | matrix metallopeptidase 26 |
| Data Set 2 | 250 gene model | 204955_at | SRPX | sushi-repeat-containing protein, X-linked |
| Data Set 2 | 250 gene model | 207397_s_at | HOXD13 | homeobox D13 |
| Data Set 2 | 250 gene model | 225721_at | SYNPO2 | synaptopodin 2 |
| Data Set 2 | 250 gene model | 225782_at | MSRB3 | methionine sulfoxide reductase B3 |
| Data Set 2 | 250 gene model | 227827_at | SORBS2 | Sorbin and SH3 domain containing 2 |
| Data Set 2 | 250 gene model | 221870_at | EHD2 | EH-domain containing 2 |
| Data Set 2 | 250 gene model | 223623_at | ECRG4 | esophageal cancer related gene 4 protein |
| Data Set 2 | 250 gene model | 225020_at | DAB2IP | DAB2 interacting protein |
| Data Set 2 | 250 gene model | 208131_s_at | PTGIS | prostaglandin I2 (prostacyclin) synthase /// prostaglandin I2 (prostacyclin) |
| | | | | synthase |
| Data Set 2 | 250 gene model | 238526_at | RAB3IP | RAB3A interacting protein (rabin3) |
| Data Set 2 | 250 gene model | 204750_s_at | DSC2 | desmocollin 2 |
| Data Set 2 | 250 gene model | 212276_at | LPIN1 | lipin 1 |
| Data Set 2 | 250 gene model | 229839_at | SCARA5 | Scavenger receptor class A, member 5 (putative) |
| Data Set 2 | 250 gene model | 230986_at | KLF8 | Kruppel-like factor 8 |
| Data Set 2 | 250 gene model | 238877_at | — | — |
| Data Set 2 | 250 gene model | 204422_s_at | FGF2 | fibroblast growth factor 2 (basic) |
| Data Set 2 | 250 gene model | 228554_at | — | MRNA; cDNA DKFZp586G0321 (from clone DKFZp586G0321) |
| Data Set 2 | 250 gene model | 204430_s_at | SLC2A5 | solute carrier family 2 (facilitated glucose/fructose transporter), member 5 |
| Data Set 2 | 250 gene model | 217728_at | S100A6 | S100 calcium binding protein A6 (calcyclin) |
| Data Set 2 | 250 gene model | 204149_s_at | GSTM4 | glutathione S-transferase M4 |
| Data Set 2 | 250 gene model | 210188_at | GABPA /// | GA binding protein transcription factor, alpha subunit 60 kDa /// GA |
| | | | GABPAP | binding protein transcription factor, alpha subunit pseudogene |
| Data Set 2 | 250 gene model | 231137_at | ACSBG1 | Acyl-CoA synthetase bubblegum family member 1 |
| Data Set 2 | 250 gene model | 226627_at | 8-Sep | septin 8 |
| Data Set 2 | 250 gene model | 201841_s_at | HSPB1 | heat shock 27 kDa protein 1 |
| Data Set 2 | 250 gene model | 227249_at | NDE1 | NudE nuclear distribution gene E homolog 1 (A. nidulans) |
| Data Set 2 | 250 gene model | 209583_s_at | CD200 | CD200 molecule |
| Data Set 2 | 250 gene model | 201348_at | GPX3 | glutathione peroxidase 3 (plasma) |
| Data Set 2 | 250 gene model | 219761_at | CLEC1A | C-type lectin domain family 1, member A |
| Data Set 2 | 250 gene model | 214247_s_at | DKK3 | dickkopf homolog 3 (Xenopus laevis) |
| Data Set 2 | 250 gene model | 224964_s_at | GNG2 | guanine nucleotide binding protein (G protein), gamma 2 |
| Data Set 2 | 250 gene model | 229313_at | — | — |
| Data Set 2 | 250 gene model | 209763_at | CHRDL1 | chordin-like 1 |
| Data Set 2 | 250 gene model | 221781_s_at | DNAJC10 | DnaJ (Hsp40) homolog, subfamily C, member 10 |
| Data Set 2 | 250 gene model | 218980_at | FHOD3 | formin homology 2 domain containing 3 |
| Data Set 2 | 250 gene model | 214121_x_at | PDLIM7 | PDZ and LIM domain 7 (enigma) |
| Data Set 2 | 250 gene model | 226834_at | — | Transcribed locus, strongly similar to NP_079045.1 adipocyte-specific |
| | | | | adhesion molecule; CAR-like membrane protein [Homo sapiens] |
| Data Set 2 | 250 gene model | 1559266_s_at | FLJ45187 | hypothetical protein LOC387640 |
| Data Set 2 | 250 gene model | 244710_at | FLJ32786 | hypothetical protein FLJ32786 |
| Data Set 2 | 250 gene model | 225912_at | TP53INP1 | tumor protein p53 inducible nuclear protein 1 |
| Data Set 2 | 250 gene model | 225464_at | FRMD6 | FERM domain containing 6 |
| Data Set 2 | 250 gene model | 210096_at | CYP4B1 | cytochrome P450, family 4, subfamily B, polypeptide 1 |
| Data Set 2 | 250 gene model | 213386_at | RNF20 | Ring finger protein 20 |
| Data Set 2 | 250 gene model | 204058_at | ME1 | Malic enzyme 1, NADP(+)-dependent, cytosolic |
| Data Set 2 | 250 gene model | 225288_at | — | Full-length cDNA clone CS0DI001YP15 of Placenta Cot 25-normalized |
| | | | | of Homo sapiens (human) |
| Data Set 2 | 250 gene model | 239503_at | — | CDNA clone IMAGE: 5301910 |
| Data Set 2 | 250 gene model | 241198_s_at | C11orf70 | chromosome 11 open reading frame 70 |
| Data Set 2 | 250 gene model | 228195_at | MGC13057 | Hypothetical protein MGC13057 |
| Data Set 2 | 250 gene model | 210105_s_at | FYN | FYN oncogene related to SRC, FGR, YES |
| Data Set 2 | 250 gene model | 205384_at | FXYD1 | FXYD domain containing ion transport regulator 1 (phospholemman) |
| Data Set 2 | 250 gene model | 225968_at | PRICKLE2 | prickle-like 2 (Drosophila) |
| Data Set 2 | 250 gene model | 220532_s_at | LR8 | LR8 protein |
| Data Set 2 | 250 gene model | 207957_s_at | PRKCB1 | Protein kinase C, beta 1 |
| Data Set 2 | 250 gene model | 206816_s_at | SPAG8 | sperm associated antigen 8 |
| Data Set 2 | 250 gene model | 200911_s_at | TACC1 | transforming, acidic coiled-coil containing protein 1 |
| Data Set 2 | 250 gene model | 226436_at | RASSF4 | Ras association (RalGDS/AF-6) domain family 4 |
| Data Set 2 | 250 gene model | 204400_at | EFS | embryonal Fyn-associated substrate |
| Data Set 2 | 250 gene model | 244289_at | LOC134466 | hypothetical protein LOC134466 |
| Data Set 2 | 250 gene model | 238484_s_at | — | MRNA; clone CD 43T7 |
| Data Set 2 | 250 gene model | 32094_at | CHST3 | carbohydrate (chondroitin 6) sulfotransferase 3 |
| Data Set 2 | 250 gene model | 228260_at | ELAVL2 | ELAV (embryonic lethal, abnormal vision, Drosophila)-like 2 (Hu antigen B) |
| Data Set 2 | 250 gene model | 204205_at | APOBEC3G | apolipoprotein B mRNA editing enzyme, catalytic polypeptide-like 3G |
| Data Set 2 | 250 gene model | 212914_at | CBX7 | chromobox homolog 7 |
| Data Set 2 | 250 gene model | 206625_at | RDS | retinal degeneration, slow |
| Data Set 2 | 250 gene model | 222666_s_at | RCL1 | RNA terminal phosphate cyclase-like 1 |
| Data Set 2 | 250 gene model | 222744_s_at | TMLHE | trimethyllysine hydroxylase, epsilon |
| Data Set 2 | 250 gene model | 219478_at | WFDC1 | WAP four-disulfide core domain 1 |
| Data Set 2 | 250 gene model | 211535_s_at | FGFR1 | fibroblast growth factor receptor 1 (fms-related tyrosine kinase 2, |
| | | | | Pfeiffer syndrome) |
| Data Set 2 | 250 gene model | 209191_at | TUBB6 | tubulin, beta 6 |
| Data Set 2 | 250 gene model | 225790_at | MSRB3 | methionine sulfoxide reductase B3 |
| Data Set 2 | 250 gene model | 238613_at | ZAK | sterile alpha motif and leucine zipper containing kinase AZK |
| Data Set 2 | 250 gene model | 241386_at | — | Transcribed locus |
| Data Set 2 | 250 gene model | 203939_at | NT5E | 5′-nucleotidase, ecto (CD73) |
| Data Set 2 | 250 gene model | 200986_at | SERPING1 | serpin peptidase inhibitor, Glade G (C1 inhibitor), member 1, (angioedema, |
| | | | | hereditary) |
| Data Set 2 | 250 gene model | 204940_at | PLN | phospholamban |
| Data Set 2 | 250 gene model | 225798_at | tcag7.981 | juxtaposed with another zinc finger gene 1 |
| Data Set 2 | 250 gene model | 222722_at | OGN | osteoglycin (osteoinductive factor, mimecan) |
| Data Set 2 | 250 gene model | 203619_s_at | FAIM2 | Fas apoptotic inhibitory molecule 2 |
| Data Set 2 | 250 gene model | 220233_at | FBXO17 | F-box protein 17 |
| Data Set 2 | 250 gene model | 231672_at | — | Transcribed locus, strongly similar to NP_057364.1 carboxylesterase 4-like; |
| | | | | carboxylesterase-related protein [Homo sapiens] |
| Data Set 2 | 250 gene model | 204894_s_at | AOC3 | amine oxidase, copper containing 3 (vascular adhesion protein 1) |
| Data Set 2 | 250 gene model | 202794_at | INPP1 | inositol polyphosphate-1-phosphatase |
| Data Set 2 | 250 gene model | 221935_s_at | C3orf64 | chromosome 3 open reading frame 64 |
| Data Set 2 | 250 gene model | 207961_x_at | MYH11 | myosin, heavy polypeptide 11, smooth muscle |
| Data Set 2 | 250 gene model | 205973_at | FEZ1 | fasciculation and elongation protein zeta 1 (zygin I) |
| Data Set 2 | 250 gene model | 223734_at | OSAP | ovary-specific acidic protein |
| Data Set 2 | 250 gene model | 228802_at | RBPMS2 | RNA binding protein with multiple splicing 2 |
| Data Set 2 | 250 gene model | 204939_s_at | PLN | phospholamban |
| Data Set 2 | 250 gene model | 227188_at | C21orf63 | chromosome 21 open reading frame 63 |
| Data Set 2 | 250 gene model | 202242_at | TSPAN7 | tetraspanin 7 |
| Data Set 2 | 250 gene model | 227915_at | ASB2 | ankyrin repeat and SOCS box-containing 2 |
| Data Set 2 | 250 gene model | 201185_at | HTRA1 | HtrA serine peptidase 1 |
| Data Set 2 | 250 gene model | 205475_at | SCRG1 | scrapie responsive protein 1 |
| Data Set 2 | 250 gene model | 203892_at | WFDC2 | WAP four-disulfide core domain 2 |
| Data Set 2 | 250 gene model | 210102_at | LOH11CR2A | loss of heterozygosity, 11, chromosomal region 2, gene A |
| Data Set 2 | 250 gene model | 228585_at | ENTPD1 | Ectonucleoside triphosphate diphosphohydrolase 1 |
| Data Set 2 | 250 gene model | 209686_at | S100B | S100 calcium binding protein, beta (neural) |
| Data Set 2 | 250 gene model | 232298_at | LOC401093 | hypothetical LOC401093 |
| Data Set 2 | 250 gene model | 212509_s_at | MXRA7 | matrix-remodelling associated 7 |
| Data Set 2 | 250 gene model | 203068_at | KLHL21 | kelch-like 21 (Drosophila) |
| Data Set 2 | 250 gene model | 65718_at | GPR124 | G protein-coupled receptor 124 |
| Data Set 2 | 250 gene model | 203729_at | EMP3 | epithelial membrane protein 3 |
| Data Set 2 | 250 gene model | 212274_at | LPIN1 | lipin 1 |
| Data Set 2 | 250 gene model | 214606_at | TSPAN2 | tetraspanin 2 |
| Data Set 2 | 250 gene model | 202796_at | SYNPO | synaptopodin |
| Data Set 2 | 250 gene model | 209343_at | EFHD1 | EF-hand domain family, member D1 |
| Data Set 2 | 250 gene model | 227115_at | — | Full-length cDNA clone CS0DF020YJ04 of Fetal brain of Homo sapiens |
| | | | | (human) |
| Data Set 2 | 250 gene model | 205573_s_at | SNX7 | sorting nexin 7 |
| Data Set 2 | 250 gene model | 208789_at | PTRF | polymerase I and transcript release factor |
| Data Set 2 | 250 gene model | 219167_at | RASL12 | RAS-like, family 12 |
| Data Set 2 | 250 gene model | 213415_at | CLIC2 | chloride intracellular channel 2 |
| Data Set 2 | 250 gene model | 205132_at | ACTC | actin, alpha, cardiac muscle |
| Data Set 2 | 250 gene model | 228807_at | — | — |
| Data Set 2 | 250 gene model | 202949_s_at | FHL2 | four and a half LIM domains 2 |
| Data Set 2 | 250 gene model | 218691_s_at | PDLIM4 | PDZ and LIM domain 4 |
| Data Set 2 | 250 gene model | 224929_at | LOC340061 | hypothetical protein LOC340061 |
| Data Set 2 | 250 gene model | 231798_at | NOG | Noggin |
| Data Set 2 | 250 gene model | 231292_at | EID3 | E1A-like inhibitor of differentiation 3 |
| Data Set 2 | 250 gene model | 227742_at | CLIC6 | chloride intracellular channel 6 |
| Data Set 2 | 250 gene model | 243481_at | RHOJ | ras homolog gene family, member J |
| Data Set 2 | 250 gene model | 236936_at | — | — |
| Data Set 2 | 250 gene model | 206194_at | HOXC4 | homeobox C4 |
| Data Set 2 | 250 gene model | 221747_at | TNS1 | Tensin 1 /// Tensin 1 |
| Data Set 2 | 250 gene model | 235737_at | TSLP | thymic stromal lymphopoietin |
| Data Set 2 | 250 gene model | 223506_at | ZC3H8 | zinc finger CCCH-type containing 8 |
| Data Set 2 | 250 gene model | 211864_s_at | FER1L3 | fer-1-like 3, myoferlin (C. elegans) |
| Data Set 2 | 250 gene model | 228202_at | PLN | Phospholamban |
| Data Set 2 | 250 gene model | 235898_at | — | Transcribed locus |
| Data Set 2 | 250 gene model | 238584_at | IQCA | IQ motif containing with AAA domain |
| Data Set 2 | 250 gene model | 207547_s_at | FAM107A | family with sequence similarity 107, member A |
| Data Set 2 | 250 gene model | 229480_at | LOC402560 | hypothetical LOC402560 |
| Data Set 2 | 250 gene model | 212886_at | CCDC69 | coiled-coil domain containing 69 |
| Data Set 2 | 250 gene model | 227976_at | LOC644538 | hypothetical protein LOC644538 |
| Data Set 2 | 250 gene model | 209434_s_at | PPAT | phosphoribosyl pyrophosphate amidotransferase |
| Data Set 2 | 250 gene model | 205083_at | AOX1 | aldehyde oxidase 1 |
| Data Set 2 | 250 gene model | 213556_at | LOC390940 | similar to R28379_1 |
| Data Set 2 | 250 gene model | 205304_s_at | KCNJ8 | potassium inwardly-rectifying channel, subfamily J, member 8 |
| Data Set 2 | 250 gene model | 227554_at | LOC402560 | Hypothetical LOC402560 |
| Data Set 2 | 250 gene model | 231118_at | ANKRD35 | ankyrin repeat domain 35 |
| Data Set 2 | 250 gene model | 230087_at | PRIMA1 | proline rich membrane anchor 1 |
| Data Set 2 | 250 gene model | 200982_s_at | ANXA6 | annexin A6 |
| Data Set 2 | 250 gene model | 1553102_a_at | CCDC69 | coiled-coil domain containing 69 |
| Data Set 2 | 250 gene model | 203324_s_at | CAV2 | caveolin 2 |
| Data Set 2 | 250 gene model | 221898_at | PDPN | podoplanin |
| Data Set 2 | 250 gene model | 235867_at | GSTM3 | glutathione S-transferase M3 (brain) |
| Data Set 2 | 250 gene model | 205303_at | KCNJ8 | potassium inwardly-rectifying channel, subfamily J, member 8 |
| Data Set 2 | 250 gene model | 209356_x_at | EFEMP2 | EGF-containing fibulin-like extracellular matrix protein 2 |
| Data Set 2 | 250 gene model | 218094_s_at | DBNDD2 | dysbindin (dystrobrevin binding protein 1) domain containing 2 |
| Data Set 2 | 250 gene model | 204777_s_at | MAL | mal, T-cell differentiation protein |
| Data Set 2 | 250 gene model | 208792_s_at | CLU | clusterin |
| Data Set 2 | 250 gene model | 242170_at | ZNF154 | Zinc finger protein 154 (pHZ-92) |
| Data Set 2 | 250 gene model | 213924_at | MPPE1 | Metallophosphoesterase 1 |
| Data Set 2 | 250 gene model | 209488_s_at | RBPMS | RNA binding protein with multiple splicing |
| Data Set 3 | 5 gene model | 1251_g_at | RAP1GAP | RAP1 GTPase activating protein |
| Data Set 3 | 5 gene model | 32565_at | SMARCD3 | SWI/SNF related, matrix associated, actin dependent regulator of |
| | | | | chromatin, subfamily d, member 3 |
| Data Set 3 | 5 gene model | 36495_at | FBP1 | fructose-1,6-bisphosphatase 1 |
| Data Set 3 | 5 gene model | 31444_s_at | ANXA2 /// | annexin A2 /// annexin A2 pseudogene 1 /// annexin A2 pseudogene 3 |
| | | | ANXA2P1 /// | |
| | | | ANXA2P3 | |
| Data Set 3 | 5 gene model | 575_s_at | TACSTD1 | tumor-associated calcium signal transducer 1 |
| Data Set 3 | 10 gene model | 36495_at | FBP1 | fructose-1,6-bisphosphatase 1 |
| Data Set 3 | 10 gene model | 33121_g_at | RGS10 | regulator of G-protein signalling 10 |
| Data Set 3 | 10 gene model | 39598_at | GJB1 | gap junction protein, beta 1, 32 kDa (connexin 32, Charcot-Marie-Tooth |
| | | | | neuropathy, X-linked) |
| Data Set 3 | 10 gene model | 36666_at | P4HB | procollagen-proline, 2-oxoglutarate 4-dioxygenase (proline 4-hydroxylase), |
| | | | | beta polypeptide |
| Data Set 3 | 10 gene model | 40060_r_at | PDLIM5 | PDZ and LIM domain 5 |
| Data Set 3 | 10 gene model | 36931_at | TAGLN | transgelin |
| Data Set 3 | 10 gene model | 34203_at | CNN1 | calponin 1, basic, smooth muscle |
| Data Set 3 | 10 gene model | 32444_at | ATP6V0E2L | ATPase, H+ transporting V0 subunit E2-like (rat) |
| Data Set 3 | 10 gene model | 32531_at | GJA1 | gap junction protein, alpha 1, 43 kDa (connexin 43) |
| Data Set 3 | 10 gene model | 34800_at | LRIG1 | leucine-rich repeats and immunoglobulin-like domains 1 |
| Data Set 3 | 20 gene model | 38098_at | LPIN1 | lipin 1 |
| Data Set 3 | 20 gene model | 691_g_at | P4HB | procollagen-proline, 2-oxoglutarate 4-dioxygenase (proline 4-hydroxylase), |
| | | | | beta polypeptide |
| Data Set 3 | 20 gene model | 36785_at | HSPB1 | heat shock 27 kDa protein 1 |
| Data Set 3 | 20 gene model | 38716_at | CAMKK2 | calcium/calmodulin-dependent protein kinase kinase 2, beta |
| Data Set 3 | 20 gene model | 35071_s_at | GMDS | GDP-mannose 4,6-dehydratase |
| Data Set 3 | 20 gene model | 36495_at | FBP1 | fructose-1,6-bisphosphatase 1 |
| Data Set 3 | 20 gene model | 35823_at | PPIB | peptidylprolyl isomerase B (cyclophilin B) |
| Data Set 3 | 20 gene model | 32135_at | SREBF1 | sterol regulatory element binding transcription factor 1 |
| Data Set 3 | 20 gene model | 38435_at | PRDX4 | peroxiredoxin 4 |
| Data Set 3 | 20 gene model | 37000_at | BRP44 | brain protein 44 |
| Data Set 3 | 20 gene model | 34885_at | SYNGR2 | synaptogyrin 2 |
| Data Set 3 | 20 gene model | 41163_at | TMED3 | transmembrane emp24 protein transport domain containing 3 |
| Data Set 3 | 20 gene model | 39965_at | RAC3 | ras-related C3 botulinum toxin substrate 3 (rho family, small GTP binding |
| | | | | protein Rac3) |
| Data Set 3 | 20 gene model | 37648_at | TTLL12 | tubulin tyrosine ligase-like family, member 12 |
| Data Set 3 | 20 gene model | 33121_g_at | RGS10 | regulator of G-protein signalling 10 |
| Data Set 3 | 20 gene model | 33396_at | GSTP1 | glutathione S-transferase pi |
| Data Set 3 | 20 gene model | 41839_at | GAS1 | growth arrest-specific 1 |
| Data Set 3 | 20 gene model | 34678_at | FER1L3 | fer-1-like 3, myoferlin (C. elegans) |
| Data Set 3 | 20 gene model | 40776_at | DES | desmin |
| Data Set 3 | 20 gene model | 41306_at | APBA2BP | amyloid beta (A4) precursor protein-binding, family A, member 2 binding |
| | | | | protein |
| Data Set 3 | 50 gene model | 37730_at | SND1 | staphylococcal nuclease domain containing 1 |
| Data Set 3 | 50 gene model | 37809_at | HOXA9 | homeobox A9 |
| Data Set 3 | 50 gene model | 36624_at | IMPDH2 | IMP (inosine monophosphate) dehydrogenase 2 |
| Data Set 3 | 50 gene model | 38044_at | FAM107A | family with sequence similarity 107, member A |
| Data Set 3 | 50 gene model | 35071_s_at | GMDS | GDP-mannose 4,6-dehydratase |
| Data Set 3 | 50 gene model | 39315_at | ANGPT1 | angiopoietin 1 |
| Data Set 3 | 50 gene model | 36791_g_at | TPM1 | tropomyosin 1 (alpha) |
| Data Set 3 | 50 gene model | 37958_at | TMEM47 | transmembrane protein 47 |
| Data Set 3 | 50 gene model | 36073_at | NDN | necdin homolog (mouse) |
| Data Set 3 | 50 gene model | 32971_at | C9orf61 | chromosome 9 open reading frame 61 |
| Data Set 3 | 50 gene model | 32542_at | FHL1 | four and a half LIM domains 1 |
| Data Set 3 | 50 gene model | 41163_at | TMED3 | transmembrane emp24 protein transport domain containing 3 |
| Data Set 3 | 50 gene model | 38719_at | NSF | N-ethylmaleimide-sensitive factor |
| Data Set 3 | 50 gene model | 41696_at | C7orf24 | chromosome 7 open reading frame 24 |
| Data Set 3 | 50 gene model | 33308_at | GUSB | glucuronidase, beta |
| Data Set 3 | 50 gene model | 41812_s_at | NUP210 | nucleoporin 210 kDa |
| Data Set 3 | 50 gene model | 41742_s_at | OPTN | optineurin |
| Data Set 3 | 50 gene model | 37917_at | FLJ20323 | hypothetical protein FLJ20323 |
| Data Set 3 | 50 gene model | 40437_at | TMEM87A | transmembrane protein 87A |
| Data Set 3 | 50 gene model | 1424_s_at | YWHAH | tyrosine 3-monooxygenase/tryptophan 5-monooxygenase activation |
| | | | | protein, eta polypeptide |
| Data Set 3 | 50 gene model | 34739_at | FNBP1L | formin binding protein 1-like |
| Data Set 3 | 50 gene model | 37000_at | BRP44 | brain protein 44 |
| Data Set 3 | 50 gene model | 37599_at | AOX1 | aldehyde oxidase 1 |
| Data Set 3 | 50 gene model | 829_s_at | GSTP1 | glutathione S-transferase pi |
| Data Set 3 | 50 gene model | 38262_at | — | Clone 23620 mRNA sequence |
| Data Set 3 | 50 gene model | 33371_s_at | RAB31 | RAB31, member RAS oncogene family |
| Data Set 3 | 50 gene model | 33611_g_at | CLDN8 | claudin 8 |
| Data Set 3 | 50 gene model | 36617_at | ID1 | inhibitor of DNA binding 1, dominant negative helix-loop-helix protein |
| Data Set 3 | 50 gene model | 40674_s_at | HOXC6 | homeobox C6 |
| Data Set 3 | 50 gene model | 661_at | GAS1 | growth arrest-specific 1 |
| Data Set 3 | 50 gene model | 38435_at | PRDX4 | peroxiredoxin 4 |
| Data Set 3 | 50 gene model | 39031_at | COX7A1 | cytochrome c oxidase subunit VIIa polypeptide 1 (muscle) |
| Data Set 3 | 50 gene model | 39099_at | SEC23A | Sec23 homolog A (S. cerevisiae) |
| Data Set 3 | 50 gene model | 32787_at | ERBB3 | v-erb-b2 erythroblastic leukemia viral oncogene homolog 3 (avian) |
| Data Set 3 | 50 gene model | 36931_at | TAGLN | transgelin |
| Data Set 3 | 50 gene model | 36432_at | MCCC2 | methylcrotonoyl-Coenzyme A carboxylase 2 (beta) |
| Data Set 3 | 50 gene model | 41745_at | IFITM3 | interferon induced transmembrane protein 3 (1-8U) |
| Data Set 3 | 50 gene model | 32314_g_at | TPM2 | tropomyosin 2 (beta) |
| Data Set 3 | 50 gene model | 36673_at | MPI | mannose phosphate isomerase |
| Data Set 3 | 50 gene model | 456_at | SMARCD3 | SWI/SNF related, matrix associated, actin dependent regulator of |
| | | | | chromatin, subfamily d, member 3 |
| Data Set 3 | 50 gene model | 34775_at | TSPAN1 | tetraspanin 1 |
| Data Set 3 | 50 gene model | 38098_at | LPIN1 | lipin 1 |
| Data Set 3 | 50 gene model | 38716_at | CAMKK2 | calcium/calmodulin-dependent protein kinase kinase 2, beta |
| Data Set 3 | 50 gene model | 1237_at | IER3 | immediate early response 3 |
| Data Set 3 | 50 gene model | 33891_at | CLIC4 | chloride intracellular channel 4 |
| Data Set 3 | 50 gene model | 39965_at | RAC3 | ras-related C3 botulinum toxin substrate 3 (rho family, small GTP |
| | | | | binding protein Rac3) |
| Data Set 3 | 50 gene model | 41306_at | APBA2BP | amyloid beta (A4) precursor protein-binding, family A, member 2 |
| | | | | binding protein |
| Data Set 3 | 50 gene model | 1257_s_at | QSCN6 | quiescin Q6 |
| Data Set 3 | 50 gene model | 41273_at | MXRA7 | matrix-remodelling associated 7 |
| Data Set 3 | 50 gene model | 38298_at | KCNMB1 | potassium large conductance calcium-activated channel, subfamily M, |
| | | | | beta member 1 |
| Data Set 3 | 100 gene model | 37043_at | ID3 | inhibitor of DNA binding 3, dominant negative helix-loop-helix protein |
| Data Set 3 | 100 gene model | 37539_at | RGL1 | ral guanine nucleotide dissociation stimulator-like 1 |
| Data Set 3 | 100 gene model | 39351_at | CD59 | CD59 molecule, complement regulatory protein |
| Data Set 3 | 100 gene model | 38422_s_at | FHL2 | four and a half LIM domains 2 |
| Data Set 3 | 100 gene model | 31684_at | ANXA2P1 | annexin A2 pseudogene 1 |
| Data Set 3 | 100 gene model | 38739_at | ETS2 | v-ets erythroblastosis virus E26 oncogene homolog 2 (avian) |
| Data Set 3 | 100 gene model | 36591_at | TUBA1 | tubulin, alpha 1 (testis specific) |
| Data Set 3 | 100 gene model | 36614_at | HSPA5 | heat shock 70 kDa protein 5 (glucose-regulated protein, 78 kDa) |
| Data Set 3 | 100 gene model | 32109_at | FXYD1 | FXYD domain containing ion transport regulator 1 (phospholemman) |
| Data Set 3 | 100 gene model | 38634_at | RBP1 | retinol binding protein 1, cellular |
| Data Set 3 | 100 gene model | 37326_at | PLP2 | proteolipid protein 2 (colonic epithelium-enriched) |
| Data Set 3 | 100 gene model | 35771_at | DEAF1 | deformed epidermal autoregulatory factor 1 (Drosophila) |
| Data Set 3 | 100 gene model | 1363_at | FGFR2 | fibroblast growth factor receptor 2 (bacteria-expressed kinase, keratinocyte |
| | | | | growth factor receptor, craniofacial dysostosis 1, Crouzon syndrome, |
| | | | | Pfeiffer syndrome, Jackson-Weiss syndrome) |
| Data Set 3 | 100 gene model | 40674_s_at | HOXC6 | homeobox C6 |
| Data Set 3 | 100 gene model | 36617_at | ID1 | inhibitor of DNA binding 1, dominant negative helix-loop-helix protein |
| Data Set 3 | 100 gene model | 38802_at | PGRMC1 | progesterone receptor membrane component 1 |
| Data Set 3 | 100 gene model | 34793_s_at | PLS3 | plastin 3 (T isoform) |
| Data Set 3 | 100 gene model | 33317_at | CDK7 | cyclin-dependent kinase 7 (MO15 homolog, Xenopus laevis, cdk-activating |
| | | | | kinase) |
| Data Set 3 | 100 gene model | 34310_at | APRT | adenine phosphoribosyltransferase |
| Data Set 3 | 100 gene model | 38328_at | SLC25A13 | solute carrier family 25, member 13 (citrin) |
| Data Set 3 | 100 gene model | 35631_at | POLR2H | polymerase (RNA) II (DNA directed) polypeptide H |
| Data Set 3 | 100 gene model | 36650_at | CCND2 | cyclin D2 |
| Data Set 3 | 100 gene model | 1814_at | TGFBR2 | transforming growth factor, beta receptor II (70/80 kDa) |
| Data Set 3 | 100 gene model | 34320_at | PTRF | polymerase I and transcript release factor |
| Data Set 3 | 100 gene model | 33610_at | CLDN8 | claudin 8 |
| Data Set 3 | 100 gene model | 38326_at | G0S2 | G0/G1switch 2 |
| Data Set 3 | 100 gene model | 212_at | ROR2 | receptor tyrosine kinase-like orphan receptor 2 |
| Data Set 3 | 100 gene model | 31693_f_at | HIST1H2AD /// | histone 1, H2ad /// histone 1, H3d |
| | | | HIST1H3D | |
| Data Set 3 | 100 gene model | 37599_at | AOX1 | aldehyde oxidase 1 |
| Data Set 3 | 100 gene model | 38921_at | PDE1B | phosphodiesterase 1B, calmodulin-dependent |
| Data Set 3 | 100 gene model | 41720_r_at | FADS1 | fatty acid desaturase 1 |
| Data Set 3 | 100 gene model | 33102_at | ADD3 | adducin 3 (gamma) |
| Data Set 3 | 100 gene model | 35071_s_at | GMDS | GDP-mannose 4,6-dehydratase |
| Data Set 3 | 100 gene model | 286_at | HIST2H2AA /// | histone 2, H2aa /// similar to Histone H2A.o (H2A/o) (H2A.2) (H2a-615) |
| | | | LOC653610 /// | /// histone H2A/r |
| | | | H2A/R | |
| Data Set 3 | 100 gene model | 32609_at | HIST2H2AA /// | histone 2, H2aa /// similar to Histone H2A.o (H2A/o) (H2A.2) (H2a-615) |
| | | | LOC653610 /// | /// histone H2A/r |
| | | | H2A/R | |
| Data Set 3 | 100 gene model | 153_f_at | HIST1H2BJ | histone 1, H2bj |
| Data Set 3 | 100 gene model | 31524_f_at | HIST1H2BI | histone 1, H2bi |
| Data Set 3 | 100 gene model | 32971_at | C9orf61 | chromosome 9 open reading frame 61 |
| Data Set 3 | 100 gene model | 32819_at | HIST1H2BK | histone 1, H2bk |
| Data Set 3 | 100 gene model | 1662_r_at | — | — |
| Data Set 3 | 100 gene model | 35127_at | HIST1H2AE | histone 1, H2ae |
| Data Set 3 | 100 gene model | 36347_f_at | HIST1H2BN | histone 1, H2bn |
| Data Set 3 | 100 gene model | 37485_at | SLC27A2 | solute carrier family 27 (fatty acid transporter), member 2 |
| Data Set 3 | 100 gene model | 37761_at | BAIAP2 | BAI1-associated protein 2 |
| Data Set 3 | 100 gene model | 31528_f_at | HIST1H2BM | histone 1, H2bm |
| Data Set 3 | 100 gene model | 1929_at | ANGPT1 | angiopoietin 1 |
| Data Set 3 | 100 gene model | 37917_at | FLJ20323 | hypothetical protein FLJ20323 |
| Data Set 3 | 100 gene model | 35576_f_at | HIST1H2BL | histone 1, H2bl |
| Data Set 3 | 100 gene model | 33308_at | GUSB | glucuronidase, beta |
| Data Set 3 | 100 gene model | 33766_at | VIPR1 | vasoactive intestinal peptide receptor 1 |
| Data Set 3 | 100 gene model | 34769_at | FAAH | fatty acid amide hydrolase |
| Data Set 3 | 100 gene model | 35628_at | TM7SF2 | transmembrane 7 superfamily member 2 |
| Data Set 3 | 100 gene model | 38719_at | NSF | N-ethylmaleimide-sensitive factor |
| Data Set 3 | 100 gene model | 35770_at | ATP6AP1 | ATPase, H+ transporting, lysosomal accessory protein 1 |
| Data Set 3 | 100 gene model | 41812_s_at | NUP210 | nucleoporin 210 kDa |
| Data Set 3 | 100 gene model | 38279_at | GNAZ | guanine nucleotide binding protein (G protein), alpha z polypeptide |
| Data Set 3 | 100 gene model | 31816_at | GAA | glucosidase, alpha; acid (Pompe disease, glycogen storage disease type II) |
| Data Set 3 | 100 gene model | 32700_at | GBP2 | guanylate binding protein 2, interferon-inducible |
| Data Set 3 | 100 gene model | 32151_at | RANGAP1 | Ran GTPase activating protein 1 |
| Data Set 3 | 100 gene model | 32526_at | JAM3 | junctional adhesion molecule 3 |
| Data Set 3 | 100 gene model | 41139_at | MAGED1 | melanoma antigen family D, 1 |
| Data Set 3 | 100 gene model | 40436_g_at | SLC25A6 | solute carrier family 25 (mitochondrial carrier; adenine nucleotide |
| | | | | translocator), member 6 |
| Data Set 3 | 100 gene model | 1980_s_at | NME2 | non-metastatic cells 2, protein (NM23B) expressed in |
| Data Set 3 | 100 gene model | 770_at | GPX3 | glutathione peroxidase 3 (plasma) |
| Data Set 3 | 100 gene model | 40069_at | SVIL | supervillin |
| Data Set 3 | 100 gene model | 37713_at | ACY1 | aminoacylase 1 |
| Data Set 3 | 100 gene model | 36073_at | NDN | necdin homolog (mouse) |
| Data Set 3 | 100 gene model | 1519_at | ETS2 | v-ets erythroblastosis virus E26 oncogene homolog 2 (avian) |
| Data Set 3 | 100 gene model | 33708_at | SLC43A1 | solute carrier family 43, member 1 |
| Data Set 3 | 100 gene model | 38218_at | GCNT1 | glucosaminyl (N-acetyl) transferase 1, core 2 (beta-1,6-N-acetyl- |
| | | | | glucosaminyltransferase) |
| Data Set 3 | 100 gene model | 39852_at | SPG20 | spastic paraplegia 20, spartin (Troyer syndrome) |
| Data Set 3 | 100 gene model | 40521_at | RGL2 | ral guanine nucleotide dissociation stimulator-like 2 |
| Data Set 3 | 100 gene model | 34050_at | ACSM1 | acyl-CoA synthetase medium-chain family member 1 |
| Data Set 3 | 100 gene model | 40435_at | SLC25A6 | solute carrier family 25 (mitochondrial carrier; adenine nucleotide |
| | | | | translocator), member 6 |
| Data Set 3 | 100 gene model | 37630_at | CHRDL1 | chordin-like 1 |
| Data Set 3 | 100 gene model | 2011_s_at | BIK | BCL2-interacting killer (apoptosis-inducing) |
| Data Set 3 | 100 gene model | 38146_at | ST18 | suppression of tumorigenicity 18 (breast carcinoma) (zinc finger protein) |
| Data Set 3 | 100 gene model | 39082_at | ANXA6 | annexin A6 |
| Data Set 3 | 100 gene model | 39243_s_at | PSIP1 | PC4 and SFRS1 interacting protein 1 |
| Data Set 3 | 100 gene model | 41814_at | FUCA1 | fucosidase, alpha-L-1, tissue |
| Data Set 3 | 100 gene model | 38044_at | FAM107A | family with sequence similarity 107, member A |
| Data Set 3 | 100 gene model | 36432_at | MCCC2 | methylcrotonoyl-Coenzyme A carboxylase 2 (beta) |
| Data Set 3 | 100 gene model | 36160_s_at | PTPRN2 | protein tyrosine phosphatase, receptor type, N polypeptide 2 |
| Data Set 3 | 100 gene model | 34739_at | FNBP1L | formin binding protein 1-like |
| Data Set 3 | 100 gene model | 36596_r_at | GATM | glycine amidinotransferase (L-arginine:glycine amidinotransferase) |
| Data Set 3 | 100 gene model | 31685_at | FEV | FEV (ETS oncogene family) |
| Data Set 3 | 100 gene model | 1911_s_at | GADD45A | growth arrest and DNA-damage-inducible, alpha |
| Data Set 3 | 100 gene model | 1424_s_at | YWHAH | tyrosine 3-monooxygenase/tryptophan 5-monooxygenase activation |
| | | | | protein, eta polypeptide |
| Data Set 3 | 100 gene model | 40301_at | GPR161 | G protein-coupled receptor 161 |
| Data Set 3 | 100 gene model | 39315_at | ANGPT1 | angiopoietin 1 |
| Data Set 3 | 100 gene model | 34213_at | WWC1 | WW, C2 and coiled-coil domain containing 1 |
| Data Set 3 | 100 gene model | 38435_at | PRDX4 | peroxiredoxin 4 |
| Data Set 3 | 100 gene model | 33900_at | FSTL3 | follistatin-like 3 (secreted glycoprotein) |
| Data Set 3 | 100 gene model | 38791_at | DDOST | dolichyl-diphosphooligosaccharide-protein glycosyltransferase |
| Data Set 3 | 100 gene model | 1597_at | GAS6 | growth arrest-specific 6 |
| Data Set 3 | 100 gene model | 41207_at | C9orf3 | chromosome 9 open reading frame 3 |
| Data Set 3 | 100 gene model | 38262_at | — | Clone 23620 mRNA sequence |
| Data Set 3 | 100 gene model | 33611_g_at | CLDN8 | claudin 8 |
| Data Set 3 | 100 gene model | 37000_at | BRP44 | brain protein 44 |
| Data Set 3 | 100 gene model | 634_at | PRSS8 | protease, serine, 8 (prostasin) |
| Data Set 3 | 250 gene model | 1248_at | POLR2H | polymerase (RNA) II (DNA directed) polypeptide H |
| Data Set 3 | 250 gene model | 36955_at | LMAN2 | lectin, mannose-binding 2 |
| Data Set 3 | 250 gene model | 33135_at | SLC19A1 | solute carrier family 19 (folate transporter), member 1 |
| Data Set 3 | 250 gene model | 41804_at | FLJ22531 | hypothetical protein FLJ22531 |
| Data Set 3 | 250 gene model | 33924_at | RAB6IP1 | RAB6 interacting protein 1 |
| Data Set 3 | 250 gene model | 40663_at | REPS2 | RALBP1 associated Eps domain containing 2 |
| Data Set 3 | 250 gene model | 40771_at | MSN | moesin |
| Data Set 3 | 250 gene model | 37939_at | APOBEC3C | apolipoprotein B mRNA editing enzyme, catalytic polypeptide-like 3C |
| Data Set 3 | 250 gene model | 36452_at | SYNPO | synaptopodin |
| Data Set 3 | 250 gene model | 37407_s_at | MYH11 | myosin, heavy polypeptide 11, smooth muscle |
| Data Set 3 | 250 gene model | 33824_at | KRT8 | keratin 8 |
| Data Set 3 | 250 gene model | 773_at | MYH11 | myosin, heavy polypeptide 11, smooth muscle |
| Data Set 3 | 250 gene model | 41137_at | PPP1R12B | protein phosphatase 1, regulatory (inhibitor) subunit 12B |
| Data Set 3 | 250 gene model | 41281_s_at | PEX10 | peroxisome biogenesis factor 10 |
| Data Set 3 | 250 gene model | 330_s_at | — | — |
| Data Set 3 | 250 gene model | 39714_at | SH3BGRL | SH3 domain binding glutamic acid-rich protein like |
| Data Set 3 | 250 gene model | 41788_i_at | TSC22D2 | TSC22 domain family, member 2 |
| Data Set 3 | 250 gene model | 36761_at | OVOL2 | ovo-like 2 (Drosophila) |
| Data Set 3 | 250 gene model | 39100_at | SPOCK1 | Sparc/osteonectin, cwcv and kazal-like domains proteoglycan (testican) 1 |
| Data Set 3 | 250 gene model | 33466_at | LOC90355 | hypothetical gene supported by AF038182; BC009203 |
| Data Set 3 | 250 gene model | 35630_at | LLGL2 | lethal giant larvae homolog 2 (Drosophila) |
| Data Set 3 | 250 gene model | 37929_at | IGSF4 | immunoglobulin superfamily, member 4 |
| Data Set 3 | 250 gene model | 39356_at | NEDD4L | neural precursor cell expressed, developmentally down-regulated 4-like |
| Data Set 3 | 250 gene model | 297_g_at | — | — |
| Data Set 3 | 250 gene model | 1270_at | RAP1GAP | RAP1 GTPase activating protein |
| Data Set 3 | 250 gene model | 32435_at | RPL19 | ribosomal protein L19 |
| Data Set 3 | 250 gene model | 35147_at | MCF2L | MCF.2 cell line derived transforming sequence-like |
| Data Set 3 | 250 gene model | 39331_at | TUBB2A | tubulin, beta 2A |
| Data Set 3 | 250 gene model | 1225_g_at | PCTK1 | PCTAIRE protein kinase 1 |
| Data Set 3 | 250 gene model | 33448_at | SPINT1 | serine peptidase inhibitor, Kunitz type 1 |
| Data Set 3 | 250 gene model | 41468_at | TRGC2 /// TRGV2 | T cell receptor gamma constant 2 /// T cell receptor gamma variable 2 /// |
| | | | /// TRGV9 /// | T cell receptor gamma variable 9 /// TCR gamma alternate reading frame |
| | | | TARP /// | protein /// hypothetical protein LOC642083 |
| | | | LOC642083 | |
| Data Set 3 | 250 gene model | 38410_at | CETN2 | centrin, EF-hand protein, 2 |
| Data Set 3 | 250 gene model | 1693_s_at | TIMP1 | TIMP metallopeptidase inhibitor 1 |
| Data Set 3 | 250 gene model | 33876_at | WWTR1 | WW domain containing transcription regulator 1 |
| Data Set 3 | 250 gene model | 40856_at | SERPINF1 | serpin peptidase inhibitor, clade F (alpha-2 antiplasmin, pigment |
| | | | | epithelium derived factor), member 1 |
| Data Set 3 | 250 gene model | 2057_g_at | FGFR1 | fibroblast growth factor receptor 1 (fms-related tyrosine kinase 2, |
| | | | | Pfeiffer syndrome) |
| Data Set 3 | 250 gene model | 37247_at | TCF21 | transcription factor 21 |
| Data Set 3 | 250 gene model | 39170_at | CD59 | CD59 molecule, complement regulatory protein |
| Data Set 3 | 250 gene model | 37576_at | PCP4 | Purkinje cell protein 4 |
| Data Set 3 | 250 gene model | 35871_s_at | SLC4A4 | solute carrier family 4, sodium bicarbonate cotransporter, member 4 |
| Data Set 3 | 250 gene model | 34955_at | ABCC4 | ATP-binding cassette, sub-family C (CFTR/MRP), member 4 |
| Data Set 3 | 250 gene model | 31528_f_at | HIST1H2BM | histone 1, H2bm |
| Data Set 3 | 250 gene model | 36790_at | TPM1 | tropomyosin 1 (alpha) |
| Data Set 3 | 250 gene model | 36533_at | PTGIS | prostaglandin I2 (prostacyclin) synthase |
| Data Set 3 | 250 gene model | 40127_at | SFXN3 | sideroflexin 3 |
| Data Set 3 | 250 gene model | 41504_s_at | MAF | v-maf musculoaponeurotic fibrosarcoma oncogene homolog (avian) |
| Data Set 3 | 250 gene model | 39544_at | DMN | desmuslin |
| Data Set 3 | 250 gene model | 501_g_at | CYP2J2 | cytochrome P450, family 2, subfamily J, polypeptide 2 |
| Data Set 3 | 250 gene model | 34684_at | RECQL | RecQ protein-like (DNA helicase Q1-like) |
| Data Set 3 | 250 gene model | 718_at | HTRA1 | HtrA serine peptidase 1 |
| Data Set 3 | 250 gene model | 35285_at | SLC4A4 | solute carrier family 4, sodium bicarbonate cotransporter, member 4 |
| Data Set 3 | 250 gene model | 39409_at | C1R /// | complement component 1, r subcomponent /// similar to Complement |
| | | | LOC643676 | C1r subcomponent precursor (Complement component 1, r subcomponent) |
| Data Set 3 | 250 gene model | 34091_s_at | VIM | vimentin |
| Data Set 3 | 250 gene model | 32535_at | FBN1 | fibrillin 1 |
| Data Set 3 | 250 gene model | 36757_at | HIST1H3H | histone 1, H3h |
| Data Set 3 | 250 gene model | 39165_at | NIFUN | NifU-like N-terminal domain containing |
| Data Set 3 | 250 gene model | 35365_at | ILK | integrin-linked kinase |
| Data Set 3 | 250 gene model | 32553_at | MAZ | MYC-associated zinc finger protein (purine-binding transcription factor) |
| Data Set 3 | 250 gene model | 32543_at | CALR | calreticulin |
| Data Set 3 | 250 gene model | 36589_at | AKR1B1 | aldo-keto reductase family 1, member B1 (aldose reductase) |
| Data Set 3 | 250 gene model | 39697_at | HSD11B2 | hydroxysteroid (11-beta) dehydrogenase 2 |
| Data Set 3 | 250 gene model | 33710_at | OACT5 | O-acyltransferase (membrane bound) domain containing 5 |
| Data Set 3 | 250 gene model | 32566_at | CHPF | chondroitin polymerizing factor |
| Data Set 3 | 250 gene model | 38831_f_at | GNB2 | guanine nucleotide binding protein (G protein), beta polypeptide 2 |
| Data Set 3 | 250 gene model | 565_at | SRD5A2 | steroid-5-alpha-reductase, alpha polypeptide 2 (3-oxo-5 alpha-steroid |
| | | | | delta 4-dehydrogenase alpha 2) |
| Data Set 3 | 250 gene model | 36204_at | PTPRF | protein tyrosine phosphatase, receptor type, F |
| Data Set 3 | 250 gene model | 38324_at | LSR | lipolysis stimulated lipoprotein receptor |
| Data Set 3 | 250 gene model | 40422_at | IGFBP2 | insulin-like growth factor binding protein 2, 36 kDa |
| Data Set 3 | 250 gene model | 32574_at | SMPD1 | sphingomyelin phosphodiesterase 1, acid lysosomal (acid |
| | | | | sphingomyelinase) |
| Data Set 3 | 250 gene model | 41368_at | SLC13A3 | solute carrier family 13 (sodium-dependent dicarboxylate transporter), |
| | | | | member 3 |
| Data Set 3 | 250 gene model | 868_at | TAF10 | TAF10 RNA polymerase II, TATA box binding protein |
| | | | | (TBP)-associated factor, 30 kDa |
| Data Set 3 | 250 gene model | 34843_at | ZNF516 | zinc finger protein 516 |
| Data Set 3 | 250 gene model | 35749_at | TADA3L | transcriptional adaptor 3 (NGG1 homolog, yeast)-like |
| Data Set 3 | 250 gene model | 1243_at | DDB2 | damage-specific DNA binding protein 2, 48 kDa |
| Data Set 3 | 250 gene model | 38292_at | HOMER2 | homer homolog 2 (Drosophila) |
| Data Set 3 | 250 gene model | 38425_at | HMGCL | 3-hydroxymethyl-3-methylglutaryl-Coenzyme A lyase |
| | | | | (hydroxymethylglutaricaciduria) |
| Data Set 3 | 250 gene model | 39752_at | CYB561D2 | cytochrome b-561 domain containing 2 |
| Data Set 3 | 250 gene model | 37016_at | ECHS1 | enoyl Coenzyme A hydratase, short chain, 1, mitochondrial |
| Data Set 3 | 250 gene model | 40570_at | FOXO1A | forkhead box O1A (rhabdomyosarcoma) |
| Data Set 3 | 250 gene model | 1135_at | GRK5 | G protein-coupled receptor kinase 5 |
| Data Set 3 | 250 gene model | 33862_at | PPAP2B | phosphatidic acid phosphatase type 2B |
| Data Set 3 | 250 gene model | 37704_at | BCKDHA | branched chain keto acid dehydrogenase E1, alpha polypeptide |
| Data Set 3 | 250 gene model | 1985_s_at | NME1 | non-metastatic cells 1, protein (NM23A) expressed in |
| Data Set 3 | 250 gene model | 32747_at | ALDH2 | aldehyde dehydrogenase 2 family (mitochondrial) |
| Data Set 3 | 250 gene model | 38408_at | TSPAN7 | tetraspanin 7 |
| Data Set 3 | 250 gene model | 36232_at | FGF13 | fibroblast growth factor 13 |
| Data Set 3 | 250 gene model | 40548_at | BICD1 | bicaudal D homolog 1 (Drosophila) |
| Data Set 3 | 250 gene model | 40775_at | ITM2A | integral membrane protein 2A |
| Data Set 3 | 250 gene model | 36690_at | NR3C1 | nuclear receptor subfamily 3, group C, member 1 (glucocorticoid receptor) |
| Data Set 3 | 250 gene model | 37225_at | ANKRD15 | ankyrin repeat domain 15 |
| Data Set 3 | 250 gene model | 39366_at | PPP1R3C | protein phosphatase 1, regulatory (inhibitor) subunit 3C |
| Data Set 3 | 250 gene model | 37343_at | ITPR3 | inositol 1,4,5-triphosphate receptor, type 3 |
| Data Set 3 | 250 gene model | 34987_s_at | HNRPA1 /// | heterogeneous nuclear ribonucleoprotein A1 /// hypothetical protein |
| | | | LOC644245 | LOC644245 |
| Data Set 3 | 250 gene model | 36676_at | RPN2 | ribophorin II |
| Data Set 3 | 250 gene model | 33253_at | TRIM14 | tripartite motif-containing 14 |
| Data Set 3 | 250 gene model | 40300_g_at | GPR161 | G protein-coupled receptor 161 |
| Data Set 3 | 250 gene model | 34695_at | SMARCD2 | SWI/SNF related, matrix associated, actin dependent regulator of chromatin, |
| | | | | subfamily d, member 2 |
| Data Set 3 | 250 gene model | 36965_at | ANK3 | ankyrin 3, node of Ranvier (ankyrin G) |
| Data Set 3 | 250 gene model | 36950_at | TMED9 | transmembrane emp24 protein transport domain containing 9 |
| Data Set 3 | 250 gene model | 33404_at | CAP2 | CAP, adenylate cyclase-associated protein, 2 (yeast) |
| Data Set 3 | 250 gene model | 38161_at | ALG3 | asparagine-linked glycosylation 3 homolog (S. cerevisiae, alpha-1,3-′ |
| | | | | mannosyltransferase) |
| Data Set 3 | 250 gene model | 37930_at | ATP7B | ATPase, Cu++ transporting, beta polypeptide |
| Data Set 3 | 250 gene model | 37022_at | PRELP | proline/arginine-rich end leucine-rich repeat protein |
| Data Set 3 | 250 gene model | 32579_at | SMARCA4 | SWI/SNF related, matrix associated, actin dependent regulator of |
| | | | | chromatin, subfamily a, member 4 |
| Data Set 3 | 250 gene model | 32246_g_at | METTL3 | methyltransferase like 3 |
| Data Set 3 | 250 gene model | 39657_at | KRT4 | keratin 4 |
| Data Set 3 | 250 gene model | 39925_at | COL9A2 | collagen, type IX, alpha 2 |
| Data Set 3 | 250 gene model | 914_g_at | ERG | v-ets erythroblastosis virus E26 oncogene like (avian) |
| Data Set 3 | 250 gene model | 1120_at | GSTM3 | glutathione S-transferase M3 (brain) |
| Data Set 3 | 250 gene model | 36147_at | SSR2 | signal sequence receptor, beta (translocon-associated protein beta) |
| Data Set 3 | 250 gene model | 36515_at | GNE | glucosamine (UDP-N-acetyl)-2-epimerase/N-acetylmannosamine kinase |
| Data Set 3 | 250 gene model | 31575_f_at | — | — |
| Data Set 3 | 250 gene model | 34699_at | CD2AP | CD2-associated protein |
| Data Set 3 | 250 gene model | 32573_at | SFRS9 | splicing factor, arginine/serine-rich 9 |
| Data Set 3 | 250 gene model | 36660_at | RAB11A | RAB11A, member RAS oncogene family |
| Data Set 3 | 250 gene model | 409_at | YWHAQ | tyrosine 3-monooxygenase/tryptophan 5-monooxygenase activation |
| | | | | protein, theta polypeptide |
| Data Set 3 | 250 gene model | 1798_at | SLC39A6 | solute carrier family 39 (zinc transporter), member 6 |
| Data Set 3 | 250 gene model | 41750_at | PDIA6 | protein disulfide isomerase family A, member 6 |
| Data Set 3 | 250 gene model | 38684_at | ATP2C1 | ATPase, Ca++ transporting, type 2C, member 1 |
| Data Set 3 | 250 gene model | 40881_at | ACLY | ATP citrate lyase |
| Data Set 3 | 250 gene model | 38041_at | GALNT1 | UDP-N-acetyl-alpha-D-galactosamine:polypeptide N-acetylgalactosaminyl- |
| | | | | transferase 1 (GalNAc-T1) |
| Data Set 3 | 250 gene model | 34823_at | DPP4 | dipeptidyl-peptidase 4 (CD26, adenosine deaminase complexing protein 2) |
| Data Set 3 | 250 gene model | 254_at | H3F3A | H3 histone, family 3A |
| Data Set 3 | 250 gene model | 32203_at | C20orf18 | chromosome 20 open reading frame 18 |
| Data Set 3 | 250 gene model | 32506_at | TBC1D1 | TBC1 (tre-2/USP6, BUB2, cdc16) domain family, member 1 |
| Data Set 3 | 250 gene model | 39023_at | IDH1 | isocitrate dehydrogenase 1 (NADP+), soluble |
| Data Set 3 | 250 gene model | 36252_at | CTF1 | cardiotrophin 1 |
| Data Set 3 | 250 gene model | 36572_r_at | ARL6IP | ADP-ribosylation factor-like 6 interacting protein |
| Data Set 3 | 250 gene model | 38010_at | BNIP3 | BCL2/adenovirus E1B 19 kDa interacting protein 3 |
| Data Set 3 | 250 gene model | 153_f_at | HIST1H2BJ | histone 1, H2bj |
| Data Set 3 | 250 gene model | 38666_at | PSCD1 | pleckstrin homology, Sec7 and coiled-coil domains 1(cytohesin 1) |
| Data Set 3 | 250 gene model | 39056_at | PAICS | phosphoribosylaminoimidazole carboxylase, phosphoribosylaminoimidazole |
| | | | | succinocarboxamide synthetase |
| Data Set 3 | 250 gene model | 31532_at | MDS1 | myelodysplasia syndrome 1 |
| Data Set 3 | 250 gene model | 32245_at | METTL3 | methyltransferase like 3 |
| Data Set 3 | 250 gene model | 32609_at | HIST2H2AA /// | histone 2, H2aa /// similar to Histone H2A.o (H2A/o) (H2A.2) (H2a-615) |
| | | | LOC653610 /// | /// histone H2A/r |
| | | | H2A/R | |
| Data Set 3 | 250 gene model | 286_at | HIST2H2AA /// | histone 2, H2aa /// similar to Histone H2A.o (H2A/o) (H2A.2) (H2a-615) |
| | | | LOC653610 /// | /// histone H2A/r |
| | | | H2A/R | |
| Data Set 3 | 250 gene model | 40607_at | DPYSL2 | dihydropyrimidinase-like 2 |
| Data Set 3 | 250 gene model | 37117_at | ARHGAP8 /// | Rho GTPase activating protein 8 /// PRR5-ARHGAP8 fusion |
| | | | LOC553158 | |
| Data Set 3 | 250 gene model | 39236_s_at | FAAH | fatty acid amide hydrolase |
| Data Set 3 | 250 gene model | 31662_at | VPS45A | vacuolar protein sorting 45A (yeast) |
| Data Set 3 | 250 gene model | 36894_at | CBX7 | chromobox homolog 7 |
| Data Set 3 | 250 gene model | 40786_at | PPP2R5C | protein phosphatase 2, regulatory subunit B (B56), gamma isoform |
| Data Set 3 | 250 gene model | 38354_at | CEBPB | CCAAT/enhancer binding protein (C/EBP), beta |
| Data Set 3 | 250 gene model | 36591_at | TUBA1 | tubulin, alpha 1 (testis specific) |
| Data Set 3 | 250 gene model | 1739_at | FOLH1 | folate hydrolase (prostate-specific membrane antigen) 1 |
| Data Set 3 | 250 gene model | 33358_at | PPM1H | protein phosphatase 1H (PP2C domain containing) |
| Data Set 3 | 250 gene model | 36963_at | PGD | phosphogluconate dehydrogenase |
| Data Set 3 | 250 gene model | 1513_at | — | — |
| Data Set 3 | 250 gene model | 1336_s_at | PRKCB1 | protein kinase C, beta 1 |
| Data Set 3 | 250 gene model | 34835_at | NCSTN | nicastrin |
| Data Set 3 | 250 gene model | 41585_at | KIAA0746 | KIAA0746 protein |
| Data Set 3 | 250 gene model | 1514_g_at | — | — |
| Data Set 3 | 250 gene model | 35615_at | BOP1 /// | block of proliferation 1 /// similar to block of proliferation 1 |
| | | | LOC653119 | |
| Data Set 3 | 250 gene model | 38614_s_at | OGT | O-linked N-acetylglucosamine (GlcNAc) transferase (UDP-N-acetyl- |
| | | | | glucosamine:polypeptide-N-acetylglucosaminyl transferase) |
| Data Set 3 | 250 gene model | 41098_at | DAAM2 | dishevelled associated activator of morphogenesis 2 |
| Data Set 3 | 250 gene model | 34840_at | SERINC5 | Serine incorporator 5 |
| Data Set 3 | 250 gene model | 36986_at | LYPLA2 | lysophospholipase II |
| Data Set 3 | 250 gene model | 32224_at | FCHSD2 | FCH and double SH3 domains 2 |
| Data Set 3 | 250 gene model | 38527_at | NONO | non-POU domain containing, octamer-binding |
| Data Set 3 | 250 gene model | 41720_r_at | FADS1 | fatty acid desaturase 1 |
| Data Set 3 | 250 gene model | 41526_at | HMG20B | high-mobility group 20B |
| Data Set 3 | 250 gene model | 38986_at | PDIA3 | protein disulfide isomerase family A, member 3 |
| Data Set 3 | 250 gene model | 35146_at | TGFB1I1 | transforming growth factor beta 1 induced transcript 1 |
| Data Set 3 | 250 gene model | 39063_at | ACTC | actin, alpha, cardiac muscle |
| Data Set 3 | 250 gene model | 40841_at | TACC1 | transforming, acidic coiled-coil containing protein 1 |
| Data Set 3 | 250 gene model | 36811_at | LOXL1 | lysyl oxidase-like 1 |
| Data Set 3 | 250 gene model | 40994_at | GRK5 | G protein-coupled receptor kinase 5 |
| Data Set 3 | 250 gene model | 37573_at | ANGPTL2 | angiopoietin-like 2 |
| Data Set 3 | 250 gene model | 36937_s_at | PDLIM1 | PDZ and LIM domain 1 (elfin) |
| Data Set 3 | 250 gene model | 37211_at | BDH1 | 3-hydroxybutyrate dehydrogenase, type 1 |
| Data Set 3 | 250 gene model | 31816_at | GAA | glucosidase, alpha; acid (Pompe disease, glycogen storage disease type II) |
| Data Set 3 | 250 gene model | 36126_at | COASY | Coenzyme A synthase |
| Data Set 3 | 250 gene model | 32798_at | GSTM3 | glutathione S-transferase M3 (brain) |
| Data Set 3 | 250 gene model | 33863_at | HYOU1 | hypoxia up-regulated 1 |
| Data Set 3 | 250 gene model | 37956_at | ALDH3B2 | aldehyde dehydrogenase 3 family, member B2 |
| Data Set 3 | 250 gene model | 39521_at | SLC12A4 | solute carrier family 12 (potassium/chloride transporters), member 4 |
| Data Set 3 | 250 gene model | 1020_s_at | CIB1 | calcium and integrin binding 1 (calmyrin) |
| Data Set 3 | 250 gene model | 34291_at | FARSLA | phenylalanine-tRNA synthetase-like, alpha subunit |
| Data Set 3 | 250 gene model | 38151_at | LOH11CR2A | loss of heterozygosity, 11, chromosomal region 2, gene A |
| Data Set 3 | 250 gene model | 40666_at | ENTPD5 | ectonucleoside triphosphate diphosphohydrolase 5 |
| Data Set 3 | 250 gene model | 1121_g_at | GSTM3 | glutathione S-transferase M3 (brain) |
| Data Set 3 | 250 gene model | 518_at | NR1H2 | nuclear receptor subfamily 1, group H, member 2 |
| Data Set 3 | 250 gene model | 35631_at | POLR2H | polymerase (RNA) II (DNA directed) polypeptide H |
| Data Set 3 | 250 gene model | 212_at | ROR2 | receptor tyrosine kinase-like orphan receptor 2 |
| Data Set 3 | 250 gene model | 37761_at | BAIAP2 | BAI1-associated protein 2 |
| Data Set 3 | 250 gene model | 37582_at | KRT15 | keratin 15 |
| Data Set 3 | 250 gene model | 32108_at | SPR | sepiapterin reductase (7,8-dihydrobiopterin:NADP+ oxidoreductase) |
| Data Set 3 | 250 gene model | 35127_at | HIST1H2AE | histone 1, H2ae |
| Data Set 3 | 250 gene model | 33362_at | CDC42EP3 | CDC42 effector protein (Rho GTPase binding) 3 |
| Data Set 3 | 250 gene model | 32544_s_at | RSU1 | Ras suppressor protein 1 |
| Data Set 3 | 250 gene model | 39781_at | IGFBP4 | insulin-like growth factor binding protein 4 |
| Data Set 3 | 250 gene model | 41870_at | PDPN | podoplanin |
| Data Set 3 | 250 gene model | 31791_at | TP73L | tumor protein p73-like |
| Data Set 3 | 250 gene model | 39753_at | ITGA5 | integrin, alpha 5 (fibronectin receptor, alpha polypeptide) |
| Data Set 3 | 250 gene model | 39123_s_at | TRPC1 | transient receptor potential cation channel, subfamily C, member 1 |
| Data Set 3 | 250 gene model | 1740_g_at | FOLH1 /// | folate hydrolase (prostate-specific membrane antigen) 1 /// growth- |
| | | | PSMAL | inhibiting protein 26 |
| Data Set 3 | 250 gene model | 31527_at | RPS2 | ribosomal protein S2 |
| Data Set 3 | 250 gene model | 35711_at | GLS2 | glutaminase 2 (liver, mitochondrial) |
| Data Set 3 | 250 gene model | 1931_at | ABCC4 | ATP-binding cassette, sub-family C (CFTR/MRP), member 4 |
| Data Set 3 | 250 gene model | 41139_at | MAGED1 | melanoma antigen family D, 1 |
| Data Set 3 | 250 gene model | 32260_at | PEA15 | phosphoprotein enriched in astrocytes 15 |
| Data Set 3 | 250 gene model | 36093_at | FLJ30092 | AF-1 specific protein phosphatase |
| Data Set 3 | 250 gene model | 38087_s_at | S100A4 | S100 calcium binding protein A4 (calcium protein, calvasculin, metastasin, |
| | | | | murine placental homolog) |
| Data Set 3 | 250 gene model | 37743_at | FEZ1 | fasciculation and elongation protein zeta 1 (zygin I) |
| Data Set 3 | 250 gene model | 296_at | — | — |
| Data Set 3 | 250 gene model | 35783_at | VAMP3 | vesicle-associated membrane protein 3 (cellubrevin) |
| Data Set 3 | 250 gene model | 38653_at | PMP22 | peripheral myelin protein 22 |
| Data Set 3 | 250 gene model | 37827_r_at | DOPEY2 | dopey family member 2 |
| Data Set 3 | 250 gene model | 37043_at | ID3 | inhibitor of DNA binding 3, dominant negative helix-loop-helix protein |
| Data Set 3 | 250 gene model | 39124_r_at | TRPC1 | transient receptor potential cation channel, subfamily C, member 1 |
| Data Set 3 | 250 gene model | 40414_at | VARS | valyl-tRNA synthetase |
| Data Set 3 | 250 gene model | 32533_s_at | VAMP5 | vesicle-associated membrane protein 5 (myobrevin) |
| Data Set 3 | 250 gene model | 33883_at | EFS | embryonal Fyn-associated substrate |
| Data Set 3 | 250 gene model | 1815_g_at | TGFBR2 | transforming growth factor, beta receptor II (70/80 kDa) |
| Data Set 3 | 250 gene model | 1585_at | ERBB3 | v-erb-b2 erythroblastic leukemia viral oncogene homolog 3 (avian) |
| Data Set 3 | 250 gene model | 1470_at | POLD2 | polymerase (DNA directed), delta 2, regulatory subunit 50 kDa |
| Data Set 3 | 250 gene model | 41223_at | COX5A | cytochrome c oxidase subunit Va |
| Data Set 3 | 250 gene model | 39396_at | LYPLA1 | lysophospholipase I |
| Data Set 3 | 250 gene model | 37680_at | AKAP12 | A kinase (PRKA) anchor protein (gravin) 12 |
| Data Set 3 | 250 gene model | 36677_at | COPB2 | coatomer protein complex, subunit beta 2 (beta prime) |
| Data Set 3 | 250 gene model | 31693_f_at | HIST1H2AD /// | histone 1, H2ad /// histone 1, H3d |
| | | | HIST1H3D | |
| Data Set 3 | 250 gene model | 36618_g_at | ID1 | inhibitor of DNA binding 1, dominant negative helix-loop-helix protein |
| Data Set 3 | 250 gene model | 34162_at | RBPMS | RNA binding protein with multiple splicing |
| Data Set 3 | 250 gene model | 924_s_at | PPP2CB | protein phosphatase 2 (formerly 2A), catalytic subunit, beta isoform |
| Data Set 3 | 250 gene model | 38780_at | AKR1A1 | aldo-keto reductase family 1, member A1 (aldehyde reductase) |
| Data Set 3 | 250 gene model | 38635_at | SSR4 | signal sequence receptor, delta (translocon-associated protein delta) |
| Data Set 3 | 250 gene model | 31524_f_at | HIST1H2BI | histone 1, H2bi |
| Data Set 3 | 250 gene model | 31684_at | ANXA2P1 | annexin A2 pseudogene 1 |
| Data Set 3 | 250 gene model | 1452_at | LMO4 | LIM domain only 4 |
| Data Set 3 | 250 gene model | 41225_at | DUSP3 | dual specificity phosphatase 3 (vaccinia virus phosphatase VH1-related) |
| Data Set 3 | 250 gene model | 40327_at | HOXB13 | homeobox B13 |
| Data Set 3 | 250 gene model | 37599_at | AOX1 | aldehyde oxidase 1 |
| Data Set 3 | 250 gene model | 33610_at | CLDN8 | claudin 8 |
| Data Set 3 | 250 gene model | 41289_at | NCAM1 | neural cell adhesion molecule 1 |
| Data Set 3 | 250 gene model | 33709_at | PDE9A | phosphodiesterase 9A |
| Data Set 3 | 250 gene model | 38396_at | — | 3′UTR of hypothetical protein (ORF1) |
| Data Set 3 | 250 gene model | 36521_at | DZIP1 | DAZ interacting protein 1 |
| Data Set 3 | 250 gene model | 38429_at | FASN | fatty acid synthase |
| Data Set 3 | 250 gene model | 33630_s_at | SPTBN2 | spectrin, beta, non-erythrocytic 2 |
| Data Set 3 | 250 gene model | 40093_at | BCAM | basal cell adhesion molecule (Lutheran blood group) |
| Data Set 3 | 250 gene model | 844_at | PPP1R1A | protein phosphatase 1, regulatory (inhibitor) subunit 1A |
| Data Set 3 | 250 gene model | 38183_at | FOXF1 | forkhead box F1 |
| Data Set 3 | 250 gene model | 34264_at | RUSC1 | RUN and SH3 domain containing 1 |
| Data Set 3 | 250 gene model | 38326_at | G0S2 | G0/G1switch 2 |
| Data Set 3 | 250 gene model | 39351_at | CD59 | CD59 molecule, complement regulatory protein |
| Data Set 3 | 250 gene model | 38921_at | PDE1B | phosphodiesterase 1B, calmodulin-dependent |
| Data Set 3 | 250 gene model | 33932_at | GSPT1 | G1 to S phase transition 1 |
| Data Set 3 | 250 gene model | 38642_at | ALCAM | activated leukocyte cell adhesion molecule |
| Data Set 3 | 250 gene model | 35742_at | C16orf45 | chromosome 16 open reading frame 45 |
| Data Set 3 | 250 gene model | 39169_at | SEC61G | Sec61 gamma subunit |
| Data Set 4 | 5 gene model | AKAP2 | | |
| Data Set 4 | 5 gene model | CAV1 | | |
| Data Set 4 | 5 gene model | TACSTD1 | | |
| Data Set 4 | 5 gene model | HPN_var1 | | |
| Data Set 4 | 5 gene model | CAMKK2 | | |
| Data Set 4 | 10 gene model | rap1GAP | | |
| Data Set 4 | 10 gene model | RAB3B | | |
| Data Set 4 | 10 gene model | TACSTD1 | | |
| Data Set 4 | 10 gene model | EXT1 | | |
| Data Set 4 | 10 gene model | TGFB3 | | |
| Data Set 4 | 10 gene model | LOC129642 | | |
| Data Set 4 | 10 gene model | SYNE1 | | |
| Data Set 4 | 10 gene model | GI_10437016 | | |
| Data Set 4 | 10 gene model | AKAP2 | | |
| Data Set 4 | 10 gene model | ITGB3 | | |
| Data Set 4 | 20 gene model | MLCK | | |
| Data Set 4 | 20 gene model | IFI27 | | |
| Data Set 4 | 20 gene model | MLP | | |
| Data Set 4 | 20 gene model | GNAZ | | |
| Data Set 4 | 20 gene model | STOM | | |
| Data Set 4 | 20 gene model | TACSTD1 | | |
| Data Set 4 | 20 gene model | KIP2 | | |
| Data Set 4 | 20 gene model | RRAS | | |
| Data Set 4 | 20 gene model | TIMP2 | | |
| Data Set 4 | 20 gene model | ILK | | |
| Data Set 4 | 20 gene model | XLKD1 | | |
| Data Set 4 | 20 gene model | EXT1 | | |
| Data Set 4 | 20 gene model | STEAP | | |
| Data Set 4 | 20 gene model | PYCR1 | | |
| Data Set 4 | 20 gene model | GSTP1 | | |
| Data Set 4 | 20 gene model | MEIS2 | | |
| Data Set 4 | 20 gene model | CDH1 | | |
| Data Set 4 | 20 gene model | RAB3B | | |
| Data Set 4 | 20 gene model | SYNE1 | | |
| Data Set 4 | 20 gene model | GI_10437016 | | |
| Data Set 4 | 50 gene model | SIAT1 | | |
| Data Set 4 | 50 gene model | GI_4884218 | | |
| Data Set 4 | 50 gene model | LIM | | |
| Data Set 4 | 50 gene model | CCK | | |
| Data Set 4 | 50 gene model | NBL1 | | |
| Data Set 4 | 50 gene model | PAICS | | |
| Data Set 4 | 50 gene model | NKX3-1 | | |
| Data Set 4 | 50 gene model | BMPR1B | | |
| Data Set 4 | 50 gene model | REPS2 | | |
| Data Set 4 | 50 gene model | IFI27 | | |
| Data Set 4 | 50 gene model | ARFIP2 | | |
| Data Set 4 | 50 gene model | D-PCa-2_mRNA | | |
| Data Set 4 | 50 gene model | ATP2C1 | | |
| Data Set 4 | 50 gene model | EDNRB | | |
| Data Set 4 | 50 gene model | BCL2_beta | | |
| Data Set 4 | 50 gene model | GI_3360414 | | |
| Data Set 4 | 50 gene model | P1 | | |
| Data Set 4 | 50 gene model | MKI67 | | |
| Data Set 4 | 50 gene model | CLU | | |
| Data Set 4 | 50 gene model | MMP2 | | |
| Data Set 4 | 50 gene model | PLS3 | | |
| Data Set 4 | 50 gene model | GALNT3 | | |
| Data Set 4 | 50 gene model | LSAMP | | |
| Data Set 4 | 50 gene model | ERBB3 | | |
| Data Set 4 | 50 gene model | LTBP4 | | |
| Data Set 4 | 50 gene model | SPARCL1 | | |
| Data Set 4 | 50 gene model | TGFB2_cds | | |
| Data Set 4 | 50 gene model | HPN_var2 | | |
| Data Set 4 | 50 gene model | KIAK0002 | | |
| Data Set 4 | 50 gene model | TNFSF10 | | |
| Data Set 4 | 50 gene model | KIAA0172 | | |
| Data Set 4 | 50 gene model | memD | | |
| Data Set 4 | 50 gene model | DNAH5 | | |
| Data Set 4 | 50 gene model | PDLIM7 | | |
| Data Set 4 | 50 gene model | SIM2 | | |
| Data Set 4 | 50 gene model | KIP2 | | |
| Data Set 4 | 50 gene model | STRA13 | | |
| Data Set 4 | 50 gene model | TGFBR3 | | |
| Data Set 4 | 50 gene model | HNF-3-alpha | | |
| Data Set 4 | 50 gene model | GNAZ | | |
| Data Set 4 | 50 gene model | EXT1 | | |
| Data Set 4 | 50 gene model | STAC | | |
| Data Set 4 | 50 gene model | MEIS2 | | |
| Data Set 4 | 50 gene model | MLP | | |
| Data Set 4 | 50 gene model | MLCK | | |
| Data Set 4 | 50 gene model | TACSTD1 | | |
| Data Set 4 | 50 gene model | XLKD1 | | |
| Data Set 4 | 50 gene model | PYCR1 | | |
| Data Set 4 | 50 gene model | STEAP | | |
| Data Set 4 | 50 gene model | CDH1 | | |
| Data Set 4 | 100 gene model | TRAF5 | | |
| Data Set 4 | 100 gene model | LIPH | | |
| Data Set 4 | 100 gene model | TP73 | | |
| Data Set 4 | 100 gene model | CALM1 | | |
| Data Set 4 | 100 gene model | TSPAN-1 | | |
| Data Set 4 | 100 gene model | SEC14L2 | | |
| Data Set 4 | 100 gene model | CD38 | | |
| Data Set 4 | 100 gene model | ROBO1 | | |
| Data Set 4 | 100 gene model | GSTM3 | | |
| Data Set 4 | 100 gene model | SLC39A6 | | |
| Data Set 4 | 100 gene model | ALDH1A2 | | |
| Data Set 4 | 100 gene model | TU3A | | |
| Data Set 4 | 100 gene model | RGS10 | | |
| Data Set 4 | 100 gene model | UB1 | | |
| Data Set 4 | 100 gene model | TRIM29 | | |
| Data Set 4 | 100 gene model | KAI1 | | |
| Data Set 4 | 100 gene model | DCC | | |
| Data Set 4 | 100 gene model | ECT2 | | |
| Data Set 4 | 100 gene model | NKX3-1 | | |
| Data Set 4 | 100 gene model | NTN1 | | |
| Data Set 4 | 100 gene model | GSTM5 | | |
| Data Set 4 | 100 gene model | IFI27 | | |
| Data Set 4 | 100 gene model | EZH2 | | |
| Data Set 4 | 100 gene model | PROK1 | | |
| Data Set 4 | 100 gene model | TRPM8 | | |
| Data Set 4 | 100 gene model | CLUL1 | | |
| Data Set 4 | 100 gene model | ZABC1 | | |
| Data Set 4 | 100 gene model | MOAT-B | | |
| Data Set 4 | 100 gene model | LIM | | |
| Data Set 4 | 100 gene model | MET | | |
| Data Set 4 | 100 gene model | NY-REN-41 | | |
| Data Set 4 | 100 gene model | KIAA0389 | | |
| Data Set 4 | 100 gene model | RPL13A | | |
| Data Set 4 | 100 gene model | PCGEM1 | | |
| Data Set 4 | 100 gene model | MAL | | |
| Data Set 4 | 100 gene model | ITPR1 | | |
| Data Set 4 | 100 gene model | GAS1 | | |
| Data Set 4 | 100 gene model | DHCR24 | | |
| Data Set 4 | 100 gene model | SPDEF | | |
| Data Set 4 | 100 gene model | SIAT1 | | |
| Data Set 4 | 100 gene model | PTTG1 | | |
| Data Set 4 | 100 gene model | MYBL2 | | |
| Data Set 4 | 100 gene model | PPP1R12A | | |
| Data Set 4 | 100 gene model | ANGPTL2 | | |
| Data Set 4 | 100 gene model | PRSS8 | | |
| Data Set 4 | 100 gene model | TGFB2 | | |
| Data Set 4 | 100 gene model | CCK | | |
| Data Set 4 | 100 gene model | HNMP-1 | | |
| Data Set 4 | 100 gene model | XBP1 | | |
| Data Set 4 | 100 gene model | SRD5A2 | | |
| Data Set 4 | 100 gene model | ANXA2 | | |
| Data Set 4 | 100 gene model | D-PCa-2_mRNA | | |
| Data Set 4 | 100 gene model | KIAA0003 | | |
| Data Set 4 | 100 gene model | SLC14A1 | | |
| Data Set 4 | 100 gene model | GDF15 | | |
| Data Set 4 | 100 gene model | HSD17B4 | | |
| Data Set 4 | 100 gene model | PAICS | | |
| Data Set 4 | 100 gene model | COL5A2 | | |
| Data Set 4 | 100 gene model | REPS2 | | |
| Data Set 4 | 100 gene model | NBL1 | | |
| Data Set 4 | 100 gene model | ARFIP2 | | |
| Data Set 4 | 100 gene model | BMPR1B | | |
| Data Set 4 | 100 gene model | D-PCa-2_var1 | | |
| Data Set 4 | 100 gene model | GJA1 | | |
| Data Set 4 | 100 gene model | DF | | |
| Data Set 4 | 100 gene model | GALNT3 | | |
| Data Set 4 | 100 gene model | PLS3 | | |
| Data Set 4 | 100 gene model | P1 | | |
| Data Set 4 | 100 gene model | HOXC6 | | |
| Data Set 4 | 100 gene model | EDNRB | | |
| Data Set 4 | 100 gene model | ZAKI-4 | | |
| Data Set 4 | 100 gene model | SYT7 | | |
| Data Set 4 | 100 gene model | TBXA2R | | |
| Data Set 4 | 100 gene model | MMP2 | | |
| Data Set 4 | 100 gene model | FBP1 | | |
| Data Set 4 | 100 gene model | AMACR | | |
| Data Set 4 | 100 gene model | SLIT3 | | |
| Data Set 4 | 100 gene model | BC008967 | | |
| Data Set 4 | 100 gene model | CNN1 | | |
| Data Set 4 | 100 gene model | KIAA0869 | | |
| Data Set 4 | 100 gene model | BIK | | |
| Data Set 4 | 100 gene model | XLKD1 | | |
| Data Set 4 | 100 gene model | CRYAB | | |
| Data Set 4 | 100 gene model | AKAP2 | | |
| Data Set 4 | 100 gene model | TMSNB | | |
| Data Set 4 | 100 gene model | HPN_var1 | | |
| Data Set 4 | 100 gene model | CAV1 | | |
| Data Set 4 | 100 gene model | ILK | | |
| Data Set 4 | 100 gene model | ITGB3 | | |
| Data Set 4 | 100 gene model | TGFB3 | | |
| Data Set 4 | 100 gene model | CAMKK2 | | |
| Data Set 4 | 100 gene model | LOC129642 | | |
| Data Set 4 | 100 gene model | PYCR1 | | |
| Data Set 4 | 100 gene model | rap1GAP | | |
| Data Set 4 | 100 gene model | ITGA5 | | |
| Data Set 4 | 100 gene model | STOM | | |
| Data Set 4 | 100 gene model | CDH1 | | |
| Data Set 4 | 100 gene model | TACSTD1 | | |
| Data Set 4 | 100 gene model | GSTP1 | | |
| Data Set 4 | 100 gene model | DNAH5 | | |
| Data Set 4 | 250 gene model | ESM1 | | |
| Data Set 4 | 250 gene model | MT3 | | |
| Data Set 4 | 250 gene model | RIG | | |
| Data Set 4 | 250 gene model | PEX5 | | |
| Data Set 4 | 250 gene model | SERPINB5 | | |
| Data Set 4 | 250 gene model | KLK2 | | |
| Data Set 4 | 250 gene model | KLK3 | | |
| Data Set 4 | 250 gene model | RET_var2 | | |
| Data Set 4 | 250 gene model | RBP1 | | |
| Data Set 4 | 250 gene model | CKTSF1B1 | | |
| Data Set 4 | 250 gene model | ODC1 | | |
| Data Set 4 | 250 gene model | BMP5 | | |
| Data Set 4 | 250 gene model | PPFIA3 | | |
| Data Set 4 | 250 gene model | HSA250839 | | |
| Data Set 4 | 250 gene model | ERBB2 | | |
| Data Set 4 | 250 gene model | SLC2A3 | | |
| Data Set 4 | 250 gene model | TRAP1 | | |
| Data Set 4 | 250 gene model | HUEL | | |
| Data Set 4 | 250 gene model | OXCT | | |
| Data Set 4 | 250 gene model | OSBPL8 | | |
| Data Set 4 | 250 gene model | PMI1 | | |
| Data Set 4 | 250 gene model | CDC42BPA | | |
| Data Set 4 | 250 gene model | BC-2 | | |
| Data Set 4 | 250 gene model | PTGDR | | |
| Data Set 4 | 250 gene model | THBS1 | | |
| Data Set 4 | 250 gene model | MMP7 | | |
| Data Set 4 | 250 gene model | CPXM | | |
| Data Set 4 | 250 gene model | NDUFA2 | | |
| Data Set 4 | 250 gene model | ITGA1 | | |
| Data Set 4 | 250 gene model | NGFB | | |
| Data Set 4 | 250 gene model | DDR1 | | |
| Data Set 4 | 250 gene model | PTOV1 | | |
| Data Set 4 | 250 gene model | LOC283431 | | |
| Data Set 4 | 250 gene model | ADAMTS1 | | |
| Data Set 4 | 250 gene model | GI_2094528 | | |
| Data Set 4 | 250 gene model | GUCY1A3 | | |
| Data Set 4 | 250 gene model | KIAA1946 | | |
| Data Set 4 | 250 gene model | HGF | | |
| Data Set 4 | 250 gene model | SPARC | | |
| Data Set 4 | 250 gene model | AKR1C3 | | |
| Data Set 4 | 250 gene model | HLTF | | |
| Data Set 4 | 250 gene model | TROAP | | |
| Data Set 4 | 250 gene model | TNFRSF6 | | |
| Data Set 4 | 250 gene model | LOX | | |
| Data Set 4 | 250 gene model | ITGB1 | | |
| Data Set 4 | 250 gene model | MAP2K1IP1 | | |
| Data Set 4 | 250 gene model | GALNT1 | | |
| Data Set 4 | 250 gene model | SND1 | | |
| Data Set 4 | 250 gene model | HNRPAB | | |
| Data Set 4 | 250 gene model | GI_1178507 | | |
| Data Set 4 | 250 gene model | D-PCa-2_var2 | | |
| Data Set 4 | 250 gene model | MMP9 | | |
| Data Set 4 | 250 gene model | PTEN | | |
| Data Set 4 | 250 gene model | MCM2 | | |
| Data Set 4 | 250 gene model | BTG2 | | |
| Data Set 4 | 250 gene model | CD44 | | |
| Data Set 4 | 250 gene model | CST3 | | |
| Data Set 4 | 250 gene model | COL1A1 | | |
| Data Set 4 | 250 gene model | PRC1 | | |
| Data Set 4 | 250 gene model | ALG-2 | | |
| Data Set 4 | 250 gene model | PGM3 | | |
| Data Set 4 | 250 gene model | C7 | | |
| Data Set 4 | 250 gene model | JUNB | | |
| Data Set 4 | 250 gene model | NIPA2 | | |
| Data Set 4 | 250 gene model | SULF1 | | |
| Data Set 4 | 250 gene model | COBLL1 | | |
| Data Set 4 | 250 gene model | PIM1 | | |
| Data Set 4 | 250 gene model | BCL2_alpha | | |
| Data Set 4 | 250 gene model | ERG_var1 | | |
| Data Set 4 | 250 gene model | CCNE2 | | |
| Data Set 4 | 250 gene model | RGS11 | | |
| Data Set 4 | 250 gene model | SFN | | |
| Data Set 4 | 250 gene model | CDH11 | | |
| Data Set 4 | 250 gene model | MME | | |
| Data Set 4 | 250 gene model | RGS5 | | |
| Data Set 4 | 250 gene model | G6PD | | |
| Data Set 4 | 250 gene model | ITSN | | |
| Data Set 4 | 250 gene model | LUM | | |
| Data Set 4 | 250 gene model | NRIP1 | | |
| Data Set 4 | 250 gene model | GI_839562 | | |
| Data Set 4 | 250 gene model | ID2 | | |
| Data Set 4 | 250 gene model | FGF18 | | |
| Data Set 4 | 250 gene model | ALDH4A1 | | |
| Data Set 4 | 250 gene model | LIPH | | |
| Data Set 4 | 250 gene model | NSP | | |
| Data Set 4 | 250 gene model | CALD1 | | |
| Data Set 4 | 250 gene model | IMPDH2 | | |
| Data Set 4 | 250 gene model | KIP | | |
| Data Set 4 | 250 gene model | DKFZp434C0931 | | |
| Data Set 4 | 250 gene model | CTHRC1 | | |
| Data Set 4 | 250 gene model | CRISP3 | | |
| Data Set 4 | 250 gene model | UCHL5 | | |
| Data Set 4 | 250 gene model | FBP1 | | |
| Data Set 4 | 250 gene model | BC008967 | | |
| Data Set 4 | 250 gene model | CRYAB | | |
| Data Set 4 | 250 gene model | AMACR | | |
| Data Set 4 | 250 gene model | KIAA0869 | | |
| Data Set 4 | 250 gene model | CNN1 | | |
| Data Set 4 | 250 gene model | AKAP2 | | |
| Data Set 4 | 250 gene model | BIK | | |
| Data Set 4 | 250 gene model | CAV1 | | |
| Data Set 4 | 250 gene model | SLIT3 | | |
| Data Set 4 | 250 gene model | TMSNB | | |
| Data Set 4 | 250 gene model | ITGB3 | | |
| Data Set 4 | 250 gene model | MEIS2 | | |
| Data Set 4 | 250 gene model | HPN_var1 | | |
| Data Set 4 | 250 gene model | XLKD1 | | |
| Data Set 4 | 250 gene model | rap1GAP | | |
| Data Set 4 | 250 gene model | MLP | | |
| Data Set 4 | 250 gene model | CAMKK2 | | |
| Data Set 4 | 250 gene model | CAV2 | | |
| Data Set 4 | 250 gene model | TGFB3 | | |
| Data Set 4 | 250 gene model | CDH1 | | |
| Data Set 4 | 250 gene model | TACSTD1 | | |
| Data Set 4 | 250 gene model | RAB3B | | |
| Data Set 4 | 250 gene model | NTRK3 | | |
| Data Set 4 | 250 gene model | KIP2 | | |
| Data Set 4 | 250 gene model | RRAS | | |
| Data Set 4 | 250 gene model | ITGA5 | | |
| Data Set 4 | 250 gene model | STEAP | | |
| Data Set 4 | 250 gene model | ILK | | |
| Data Set 4 | 250 gene model | KIAA0172 | | |
| Data Set 4 | 250 gene model | SYNE1 | | |
| Data Set 4 | 250 gene model | GNAZ | | |
| Data Set 4 | 250 gene model | PYCR1 | | |
| Data Set 4 | 250 gene model | LOC129642 | | |
| Data Set 4 | 250 gene model | MMP2 | | |
| Data Set 4 | 250 gene model | EXT1 | | |
| Data Set 4 | 250 gene model | GSTP1 | | |
| Data Set 4 | 250 gene model | ERBB3 | | |
| Data Set 4 | 250 gene model | GI_10437016 | | |
| Data Set 4 | 250 gene model | STOM | | |
| Data Set 4 | 250 gene model | STAC | | |
| Data Set 4 | 250 gene model | FOLH1 | | |
| Data Set 4 | 250 gene model | DNAH5 | | |
| Data Set 4 | 250 gene model | TIMP2 | | |
| Data Set 4 | 250 gene model | PDLIM7 | | |
| Data Set 4 | 250 gene model | TGFBR3 | | |
| Data Set 4 | 250 gene model | HNF-3-alpha | | |
| Data Set 4 | 250 gene model | SIM2 | | |
| Data Set 4 | 250 gene model | MLCK | | |
| Data Set 4 | 250 gene model | memD | | |
| Data Set 4 | 250 gene model | TNFSF10 | | |
| Data Set 4 | 250 gene model | KIAK0002 | | |
| Data Set 4 | 250 gene model | MAL | | |
| Data Set 4 | 250 gene model | STRA13 | | |
| Data Set 4 | 250 gene model | ARFIP2 | | |
| Data Set 4 | 250 gene model | MKI67 | | |
| Data Set 4 | 250 gene model | TBXA2R | | |
| Data Set 4 | 250 gene model | ZAKI-4 | | |
| Data Set 4 | 250 gene model | BCL2_beta | | |
| Data Set 4 | 250 gene model | CLU | | |
| Data Set 4 | 250 gene model | P1 | | |
| Data Set 4 | 250 gene model | GALNT3 | | |
| Data Set 4 | 250 gene model | GAS1 | | |
| Data Set 4 | 250 gene model | COL5A2 | | |
| Data Set 4 | 250 gene model | LTBP4 | | |
| Data Set 4 | 250 gene model | PLS3 | | |
| Data Set 4 | 250 gene model | GI_4884218 | | |
| Data Set 4 | 250 gene model | SYT7 | | |
| Data Set 4 | 250 gene model | HPN_var2 | | |
| Data Set 4 | 250 gene model | TGFB2_cds | | |
| Data Set 4 | 250 gene model | HOXC6 | | |
| Data Set 4 | 250 gene model | PAICS | | |
| Data Set 4 | 250 gene model | LSAMP | | |
| Data Set 4 | 250 gene model | NBL1 | | |
| Data Set 4 | 250 gene model | GDF15 | | |
| Data Set 4 | 250 gene model | ITPR1 | | |
| Data Set 4 | 250 gene model | REPS2 | | |
| Data Set 4 | 250 gene model | ANGPTL2 | | |
| Data Set 4 | 250 gene model | BMPR1B | | |
| Data Set 4 | 250 gene model | GI_3360414 | | |
| Data Set 4 | 250 gene model | ATP2C1 | | |
| Data Set 4 | 250 gene model | RPL13A | | |
| Data Set 4 | 250 gene model | SPARCL1 | | |
| Data Set 4 | 250 gene model | PRSS8 | | |
| Data Set 4 | 250 gene model | SLC14A1 | | |
| Data Set 4 | 250 gene model | DF | | |
| Data Set 4 | 250 gene model | D-PCa-2_mRNA | | |
| Data Set 4 | 250 gene model | EDNRB | | |
| Data Set 4 | 250 gene model | SIAT1 | | |
| Data Set 4 | 250 gene model | D-PCa-2_var1 | | |
| Data Set 4 | 250 gene model | XBP1 | | |
| Data Set 4 | 250 gene model | KIAA0003 | | |
| Data Set 4 | 250 gene model | VCL | | |
| Data Set 4 | 250 gene model | KIAA0389 | | |
| Data Set 4 | 250 gene model | HNMP-1 | | |
| Data Set 4 | 250 gene model | MOAT-B | | |
| Data Set 4 | 250 gene model | SRD5A2 | | |
| Data Set 4 | 250 gene model | PPP1R12A | | |
| Data Set 4 | 250 gene model | IFI27 | | |
| Data Set 4 | 250 gene model | PCGEM1 | | |
| Data Set 4 | 250 gene model | ZABC1 | | |
| Data Set 4 | 250 gene model | HSD17B4 | | |
| Data Set 4 | 250 gene model | PPAP2B | | |
| Data Set 4 | 250 gene model | SPDEF | | |
| Data Set 4 | 250 gene model | TP73 | | |
| Data Set 4 | 250 gene model | RGS10 | | |
| Data Set 4 | 250 gene model | ANXA2 | | |
| Data Set 4 | 250 gene model | DHCR24 | | |
| Data Set 4 | 250 gene model | CCK | | |
| Data Set 4 | 250 gene model | NY-REN-41 | | |
| Data Set 4 | 250 gene model | MYBL2 | | |
| Data Set 4 | 250 gene model | NTN1 | | |
| Data Set 4 | 250 gene model | NKX3-1 | | |
| Data Set 4 | 250 gene model | TGFB2 | | |
| Data Set 4 | 250 gene model | GJA1 | | |
| Data Set 4 | 250 gene model | MET | | |
| Data Set 4 | 250 gene model | EZH2 | | |
| Data Set 4 | 250 gene model | PTTG1 | | |
| Data Set 4 | 250 gene model | FZD7 | | |
| Data Set 4 | 250 gene model | TRPM8 | | |
| Data Set 4 | 250 gene model | DCC | | |
| Data Set 4 | 250 gene model | UB1 | | |
| Data Set 4 | 250 gene model | CLUL1 | | |
| Data Set 4 | 250 gene model | LIM | | |
| Data Set 4 | 250 gene model | SCUBE2 | | |
| Data Set 4 | 250 gene model | tom1-like | | |
| Data Set 4 | 250 gene model | TSPAN-1 | | |
| Data Set 4 | 250 gene model | SEC14L2 | | |
| Data Set 4 | 250 gene model | SERPINF1 | | |
| Data Set 4 | 250 gene model | GSTM5 | | |
| Data Set 4 | 250 gene model | CALM1 | | |
| Data Set 4 | 250 gene model | DAT1 | | |
| Data Set 4 | 250 gene model | MCCC2 | | |
| Data Set 4 | 250 gene model | BNIP3 | | |
| Data Set 4 | 250 gene model | TFAP2C | | |
| Data Set 4 | 250 gene model | KAI1 | | |
| Data Set 4 | 250 gene model | TGFB1 | | |
| Data Set 4 | 250 gene model | NEFH | | |
| Data Set 4 | 250 gene model | ALDH1A2 | | |
| Data Set 4 | 250 gene model | ECT2 | | |
| Data Set 4 | 250 gene model | COL4A2 | | |
| Data Set 4 | 250 gene model | TU3A | | |
| Data Set 4 | 250 gene model | CHAF1A | | |
| Data Set 4 | 250 gene model | CD38 | | |
| Data Set 4 | 250 gene model | CES1 | | |
| Data Set 4 | 250 gene model | DKFZP564B167 | | |
| Data Set 4 | 250 gene model | STEAP2 | | |
| Data Set 4 | 250 gene model | COL4A1 | | |
| Data Set 4 | 250 gene model | SLC39A6 | | |
| Data Set 4 | 250 gene model | UNC5C | | |
| Data Set 4 | 250 gene model | TMEPAI | | |
| Data Set 4 | 250 gene model | GI_2056367 | | |
| Data Set 4 | 250 gene model | Prostein | | |
| Data Set 4 | 250 gene model | GPR43 | | |
| Data Set 4 | 250 gene model | GI_22761402 | | |
| Data Set 4 | 250 gene model | PROK1 | | |
| Data Set 4 | 250 gene model | TRIM29 | | |
| Data Set 4 | 250 gene model | ANTXR1 | | |

TABLE 19
In silico tissue components (tumor/stroma) prediction discrepancies (%) and
correlation coefficients compared to pathologist's estimates across data sets.

| Test | Training Set | | | |
|---|---|---|---|---|
| Set | Data Set 1 | Data Set 2 | Data Set 3 | Data Set 4 |
| Data Set 1 | NA | 11.6/11.8(0.82/0.73) | 23.7/27(0.86/0.74) | 13.3/18.8(0.82/0.75) |
| Data Set 2 | 11/16.7(0.89/0.76) | NA | 22.1/38.2(0.84/0.63) | 28.6/25.8(0.79/0.72) |
| Data Set 3 | 14.5/15.1(0.76/0.64 | 13.7/22.3(0.75/0.59) | NA | 17.4/14.7(0.71/0.59) |
| Data Set 4 | 12.1/24.5(0.76/0.62) | 12.7/23.7(0.73/0.62) | 12.8/19.9(0.72/0.61) | NA |

### Example 4

### Identification of Tissue Specific Genes in Prostate Cancer

Genes specifically expressed in different cell types (tumor, stroma, BPH and atrophic gland) of prostate tissue were identified.

### Tissue Content Prediction Using Gene Expression Profile

Using linear models based on a small list of tissue specific genes, the tissue components of samples hybridized to the array is predictable. These genes are listed in Table 20.

### Tissue Specific Relapse Related Genes

Some tissue specific genes showed significant expression level changes between relapse and non-relapse samples. The gene list is shown in Table 8 above.

TABLE 20
Tissue specific genes for tissue prediction.

| Tissue Type | | | Gene | RefSeq | Rep. | UniGene |
|---|---|---|---|---|---|---|
| Predicted | U133A ID | Gene Title | Symbol | Transcript ID | Public ID | ID |
| Tumor | 211194_s_at | tumor protein p73- | TP73L | NM_003722 | AB010153 | Hs.137569 |
| | | like | | | | |
| Tumor | 202310_s_at | collagen, type I, | COL1A1 | NM_000088 | K01228 | Hs.172928 |
| | | alpha 1 | | | | |
| Tumor | 216062_at | CD44 molecule | CD44 | NM_000610 /// | AW851559 | Hs.502328 |
| | | (Indian blood | | NM_001001389 | | |
| | | group) | | /// | | |
| | | | | NM_001001390 | | |
| | | | | /// | | |
| | | | | NM_001001391 | | |
| | | | | /// | | |
| | | | | NM_001001392 | | |
| Tumor | 211872_s_at | regulator of G- | RGS11 | NM_003834 /// | AB016929 | Hs.65756 |
| | | protein signalling | | NM_183337 | | |
| | | 11 | | | | |
| Tumor | 215240_at | integrin, beta 3 | ITGB3 | NM_000212 | AI189839 | Hs.218040 |
| | | (platelet | | | | |
| | | glycoprotein IIIa, | | | | |
| | | antigen CD61) | | | | |
| Tumor | 204748_at | prostaglandin- | PTGS2 | NM_000963 | NM_000963 | Hs.196384 |
| | | endoperoxide | | | | |
| | | synthase 2 | | | | |
| | | (prostaglandin G/H | | | | |
| | | synthase and | | | | |
| | | cyclooxygenase) | | | | |
| Tumor | 204926_at | inhibin, beta A | INHBA | NM_002192 | NM_002192 | Hs.583348 |
| | | (activin A, activin | | | | |
| | | AB alpha | | | | |
| | | polypeptide) | | | | |
| Tumor | 205042_at | glucosamine | GNE | NM_005476 | NM_005476 | Hs.5920 |
| | | (UDP-N-acetyl)-2- | | | | |
| | | epimerase/N- | | | | |
| | | acetylmannosamine | | | | |
| | | kinase | | | | |
| Tumor | 222043_at | clusterin | CLU | NM_001831 /// | AI982754 | Hs.436657 |
| | | | | NM_203339 | | |
| Tumor | 212984_at | activating | ATF2 | NM_001880 | BE786164 | Hs.591614 |
| | | transcription factor 2 | | | | |
| Tumor | 215775_at | Thrombospondin 1 | THBS1 | NM_003246 | BF084105 | Hs.164226 |
| Tumor | 204742_s_at | androgen-induced | APRIN | NM_015032 | NM_015032 | Hs.567425 |
| | | proliferation | | | | |
| | | inhibitor | | | | |
| Tumor | 203698_s_at | frizzled-related | FRZB | NM_001463 | NM_001463 | Hs.128453 |
| | | protein | | | | |
| Tumor | 209771_x_at | CD24 molecule | CD24 | NM_013230 | AA761181 | Hs.632285 |
| Tumor | 201839_s_at | tumor-associated | TACSTD1 | NM_002354 | NM_002354 | Hs.542050 |
| | | calcium signal | | | | |
| | | transducer 1 | | | | |
| Tumor | 205834_s_at | Prostate androgen- | PART1 | — | NM_016590 | Hs.146312 |
| | | regulated transcript 1 | | | | |
| Tumor | 209935_at | ATPase, Ca++ | ATP2C1 | NM_001001485 | AF225981 | Hs.584884 |
| | | transporting, type | | /// | | |
| | | 2C, member 1 | | NM_001001486 | | |
| | | | | /// | | |
| | | | | NM_001001487 | | |
| | | | | /// NM_014382 | | |
| Tumor | 211834_s_at | tumor protein p73- | TP73L | NM_003722 | AB042841 | Hs.137569 |
| | | like | | | | |
| Tumor | 210930_s_at | v-erb-b2 | ERBB2 | NM_001005862 | AF177761 | Hs.446352 |
| | | erythroblastic | | /// NM_004448 | | |
| | | leukemia viral | | | | |
| | | oncogene homolog | | | | |
| | | 2, | | | | |
| | | neuro/glioblastoma | | | | |
| | | derived oncogene | | | | |
| | | homolog (avian) | | | | |
| Tumor | 212230_at | phosphatidic acid | PPAP2B | NM_003713 /// | AV725664 | Hs.405156 |
| | | phosphatase type | | NM_177414 | | |
| | | 2B | | | | |
| Tumor | 202089_s_at | solute carrier | SLC39A6 | NM_012319 | NM_012319 | Hs.79136 |
| | | family 39 (zinc | | | | |
| | | transporter), | | | | |
| | | member 6 | | | | |
| Tumor | 201409_s_at | protein | PPP1CB | NM_002709 /// | NM_002709 | Hs.591571 |
| | | phosphatase 1, | | NM_206876 /// | | |
| | | catalytic subunit, | | NM_206877 | | |
| | | beta isoform | | | | |
| Tumor | 201555_at | MCM3 | MCM3 | NM_002388 | NM_002388 | Hs.179565 |
| | | minichromosome | | | | |
| | | maintenance | | | | |
| | | deficient 3 (S. cerevisiae) | | | | |
| Tumor | 217487_x_at | folate hydrolase | FOLH1 | NM_001014986 | AF254357 | Hs.380325 |
| | | (prostate-specific | | /// NM_004476 | | |
| | | membrane antigen) 1 | | | | |
| Tumor | 201744_s_at | lumican | LUM | NM_002345 | NM_002345 | Hs.406475 |
| Tumor | 201215_at | plastin 3 (T | PLS3 | NM_005032 | NM_005032 | Hs.496622 |
| | | isoform) | | | | |
| Tumor | 211748_x_at | prostaglandin D2 | PTGDS | NM_000954 | BC005939 | Hs.446429 |
| | | synthase 21 kDa | | | | |
| | | (brain) /// | | | | |
| | | prostaglandin D2 | | | | |
| | | synthase 21 kDa | | | | |
| | | (brain) | | | | |
| Tumor | 221788_at | Phosphoglucomutase 3 | PGM3 | NM_015599 | AV727934 | Hs.598312 |
| Tumor | 215564_at | Amphiregulin | AREG | NM_001657 | AV652031 | Hs.270833 |
| | | (schwannoma- | | | | |
| | | derived growth | | | | |
| | | factor) | | | | |
| Tumor | 211964_at | collagen, type IV, | COL4A2 | NM_001846 | X05610 | Hs.508716 |
| | | alpha 2 | | | | |
| Tumor | 201739_at | serum/glucocorticoid | SGK | NM_005627 | NM_005627 | Hs.510078 |
| | | regulated kinase | | | | |
| Tumor | 209854_s_at | kallikrein 2, | KLK2 | NM_001002231 | AA595465 | Hs.515560 |
| | | prostatic | | /// | | |
| | | | | NM_001002232 | | |
| | | | | /// NM_005551 | | |
| Tumor | 33322_i_at | stratifin | SFN | NM_006142 | X57348 | Hs.523718 |
| Tumor | 205780_at | BCL2-interacting | BIK | NM_001197 | NM_001197 | Hs.475055 |
| | | killer (apoptosis- | | | | |
| | | inducing) | | | | |
| Tumor | 201577_at | non-metastatic | NME1 | NM_000269 /// | NM_000269 | Hs.463456 |
| | | cells 1, protein | | NM_198175 | | |
| | | (NM23A) | | | | |
| | | expressed in | | | | |
| Tumor | 209706_at | NK3 transcription | NKX3-1 | NM_006167 | AF247704 | Hs.55999 |
| | | factor related, | | | | |
| | | locus 1 | | | | |
| | | (Drosophila) | | | | |
| Tumor | 200931_s_at | vinculin | VCL | NM_003373 /// | NM_014000 | Hs.500101 |
| | | | | NM_014000 | | |
| Tumor | 202436_s_at | cytochrome P450, | CYP1B1 | NM_000104 | AU144855 | Hs.154654 |
| | | family 1, | | | | |
| | | subfamily B, | | | | |
| | | polypeptide 1 | | | | |
| Tumor | 209283_at | crystallin, alpha B | CRYAB | NM_001885 | AF007162 | Hs.408767 |
| Tumor | 202088_at | solute carrier | SLC39A6 | NM_012319 | AI635449 | Hs.79136 |
| | | family 39 (zinc | | | | |
| | | transporter), | | | | |
| | | member 6 | | | | |
| Tumor | 215350_at | spectrin repeat | SYNE1 | NM_015293 /// | AB033088 | Hs.12967 |
| | | containing, nuclear | | NM_033071 /// | | |
| | | envelope 1 | | NM_133650 /// | | |
| | | | | NM_182961 | | |
| Stroma | 202088_at | solute carrier | SLC39A6 | NM_012319 | AI635449 | Hs.79136 |
| | | family 39 (zinc | | | | |
| | | transporter), | | | | |
| | | member 6 | | | | |
| Stroma | 200931_s_at | vinculin | VCL | NM_003373 /// | NM_014000 | Hs.500101 |
| | | | | NM_014000 | | |
| Stroma | 209854_s_at | kallikrein 2, | KLK2 | NM_001002231 | AA595465 | Hs.515560 |
| | | prostatic | | /// | | |
| | | | | NM_001002232 | | |
| | | | | /// NM_005551 | | |
| Stroma | 205780_at | BCL2-interacting | BIK | NM_001197 | NM_001197 | Hs.475055 |
| | | killer (apoptosis- | | | | |
| | | inducing) | | | | |
| Stroma | 217487_x_at | folate hydrolase | FOLH1 | NM_001014986 | AF254357 | Hs.380325 |
| | | (prostate-specific | | /// NM_004476 | | |
| | | membrane antigen) 1 | | | | |
| Stroma | 221788_at | Phosphoglucomutase 3 | PGM3 | NM_015599 | AV727934 | Hs.598312 |
| Stroma | 202089_s_at | solute carrier | SLC39A6 | NM_012319 | NM_012319 | Hs.79136 |
| | | family 39 (zinc | | | | |
| | | transporter), | | | | |
| | | member 6 | | | | |
| Stroma | 211194_s_at | tumor protein p73- | TP73L | NM_003722 | AB010153 | Hs.137569 |
| | | like | | | | |
| BPH | 205659_at | histone deacetylase 9 | HDAC9 | NM_014707 /// | NM_014707 | Hs.196054 |
| | | | | NM_058176 /// | | |
| | | | | NM_058177 /// | | |
| | | | | NM_178423 /// | | |
| | | | | NM_178425 | | |
| BPH | 215350_at | spectrin repeat | SYNE1 | NM_015293 /// | AB033088 | Hs.12967 |
| | | containing, nuclear | | NM_033071 /// | | |
| | | envelope 1 | | NM_133650 /// | | |
| | | | | NM_182961 | | |
| BPH | 201577_at | non-metastatic | NME1 | NM_000269 /// | NM_000269 | Hs.463456 |
| | | cells 1, protein | | NM_198175 | | |
| | | (NM23A) | | | | |
| | | expressed in | | | | |
| BPH | 215564_at | Amphiregulin | AREG | NM_001657 | AV652031 | Hs.270833 |
| | | (schwannoma- | | | | |
| | | derived growth | | | | |
| | | factor) | | | | |
| BPH | 210984_x_at | epidermal growth | EGFR | NM_005228 /// | U95089 | Hs.488293 |
| | | factor receptor | | NM_201282 /// | | |
| | | (erythroblastic | | NM_201283 /// | | |
| | | leukemia viral (v- | | NM_201284 | | |
| | | erb-b) oncogene | | | | |
| | | homolog, avian) | | | | |
| BPH | 33322_i_at | stratifin | SFN | NM_006142 | X57348 | Hs.523718 |
| BPH | 202312_s_at | collagen, type I, | COL1A1 | NM_000088 | NM_000088 | Hs.172928 |
| | | alpha 1 | | | | |
| BPH | 211834_s_at | tumor protein p73- | TP73L | NM_003722 | AB042841 | Hs.137569 |
| | | like | | | | |
| BPH | 204777_s_at | mal, T-cell | MAL | NM_002371 /// | NM_002371 | Hs.80395 |
| | | differentiation | | NM_022438 /// | | |
| | | protein | | NM_022439 /// | | |
| | | | | NM_022440 | | |
| BPH | 201667_at | gap junction | GJA1 | NM_000165 | NM_000165 | Hs.74471 |
| | | protein, alpha 1, | | | | |
| | | 43 kDa (connexin | | | | |
| | | 43) | | | | |
| BPH | 202436_s_at | cytochrome P450, | CYP1B1 | NM_000104 | AU144855 | Hs.154654 |
| | | family 1, | | | | |
| | | subfamily B, | | | | |
| | | polypeptide 1 | | | | |
| BPH | 210930_s_at | v-erb-b2 | ERBB2 | NM_001005862 | AF177761 | Hs.446352 |
| | | erythroblastic | | /// NM_004448 | | |
| | | leukemia viral | | | | |
| | | oncogene homolog | | | | |
| | | 2, | | | | |
| | | neuro/glioblastoma | | | | |
| | | derived oncogene | | | | |
| | | homolog (avian) | | | | |
| BPH | 214403_x_at | SAM pointed | SPDEF | NM_012391 | AI307915 | Hs.485158 |
| | | domain containing | | | | |
| | | ets transcription | | | | |
| | | factor | | | | |
| BPH | 212230_at | phosphatidic acid | PPAP2B | NM_003713 /// | AV725664 | Hs.405156 |
| | | phosphatase type | | NM_177414 | | |
| | | 2B | | | | |
| BPH | 33767_at | neurofilament, | NEFH | NM_021076 | X15306 | Hs.198760 |
| | | heavy polypeptide | | | | |
| | | 200 kDa | | | | |
| BPH | 200931_s_at | vinculin | VCL | NM_003373 /// | NM_014000 | Hs.500101 |
| | | | | NM_014000 | | |
| BPH | 217995_at | sulfide quinone | SQRDL | NM_021199 | NM_021199 | Hs.511251 |
| | | reductase-like | | | | |
| | | (yeast) | | | | |
| BPH | 204734_at | keratin 15 | KRT15 | NM_002275 | NM_002275 | — |
| BPH | 209706_at | NK3 transcription | NKX3-1 | NM_006167 | AF247704 | Hs.55999 |
| | | factor related, | | | | |
| | | locus 1 | | | | |
| | | (Drosophila) | | | | |
| BPH | 214399_s_at | Keratin 8 | KRT8 | NM_002273 | BF588953 | Hs.533782 |
| BPH | 211964_at | collagen, type IV, | COL4A2 | NM_001846 | X05610 | Hs.508716 |
| | | alpha 2 | | | | |
| BPH | 203372_s_at | suppressor of | SOCS2 | NM_003877 | AB004903 | Hs.485572 |
| | | cytokine signaling 2 | | | | |
| BPH | 211156_at | cyclin-dependent | CDKN2A | NM_000077 /// | AF115544 | Hs.512599 |
| | | kinase inhibitor 2A | | NM_058195 /// | | |
| | | (melanoma, p16, | | NM_058197 | | |
| | | inhibits CDK4) | | | | |
| BPH | 205780_at | BCL2-interacting | BIK | NM_001197 | NM_001197 | Hs.475055 |
| | | killer (apoptosis- | | | | |
| | | inducing) | | | | |
| BPH | 212142_at | MCM4 | MCM4 | NM_005914 /// | AI936566 | Hs.460184 |
| | | minichromosome | | NM_182746 | | |
| | | maintenance | | | | |
| | | deficient 4 (S. cerevisiae) | | | | |
| BPH | 201130_s_at | cadherin 1, type 1, | CDH1 | NM_004360 | L08599 | Hs.461086 |
| | | E-cadherin | | | | |
| | | (epithelial) | | | | |
| BPH | 201109_s_at | thrombospondin 1 | THBS1 | NM_003246 | AV726673 | Hs.164226 |
| BPH | 215775_at | Thrombospondin 1 | THBS1 | NM_003246 | BF084105 | Hs.164226 |
| BPH | 201262_s_at | biglycan | BGN | NM_001711 | NM_001711 | Hs.821 |
| BPH | 204625_s_at | integrin, beta 3 | ITGB3 | NM_000212 | BF115658 | Hs.218040 |
| | | (platelet | | | | |
| | | glycoprotein IIIa, | | | | |
| | | antigen CD61) | | | | |
| BPH | 216062_at | CD44 molecule | CD44 | NM_000610 /// | AW851559 | Hs.502328 |
| | | (Indian blood | | NM_001001389 | | |
| | | group) | | /// | | |
| | | | | NM_001001390 | | |
| | | | | /// | | |
| | | | | NM_001001391 | | |
| | | | | /// | | |
| | | | | NM_001001392 | | |
| BPH | 222043_at | clusterin | CLU | NM_001831 /// | AI982754 | Hs.436657 |
| | | | | NM_203339 | | |
| BPH | 204748_at | prostaglandin- | PTGS2 | NM_000963 | NM_000963 | Hs.196384 |
| | | endoperoxide | | | | |
| | | synthase 2 | | | | |
| | | (prostaglandin G/H | | | | |
| | | synthase and | | | | |
| | | cyclooxygenase) | | | | |
| BPH | 215240_at | integrin, beta 3 | ITGB3 | NM_000212 | AI189839 | Hs.218040 |
| | | (platelet | | | | |
| | | glycoprotein IIIa, | | | | |
| | | antigen CD61) | | | | |
| BPH | 219197_s_at | signal peptide, | SCUBE2 | NM_020974 | AI424243 | Hs.523468 |
| | | CUB domain, | | | | |
| | | EGF-like 2 | | | | |
| BPH | 211194_s_at | tumor protein p73- | TP73L | NM_003722 | AB010153 | Hs.137569 |
| | | like | | | | |
| Tumor | 214460_at | limbic system- | LSAMP | NM_002338 | NM_002338 | Hs.26479 |
| | | associated | | | | |
| | | membrane protein | | | | |
| Tumor | 201394_s_at | RNA binding | RBM5 | NM_005778 | U23946 | Hs.439480 |
| | | motif protein 5 | | | | |
| Tumor | 202525_at | protease, serine, 8 | PRSS8 | NM_002773 | NM_002773 | Hs.75799 |
| | | (prostasin) | | | | |
| Tumor | 201577_at | non-metastatic | NME1 | NM_000269 /// | NM_000269 | Hs.463456 |
| | | cells 1, protein | | NM_198175 | | |
| | | (NM23A) | | | | |
| | | expressed in | | | | |
| Tumor | 205645_at | RALBP1 | REPS2 | NM_004726 | NM_004726 | Hs.186810 |
| | | associated Eps | | | | |
| | | domain containing 2 | | | | |
| Tumor | 203425_s_at | insulin-like growth | IGFBP5 | NM_000599 | NM_000599 | Hs.369982 |
| | | factor binding | | | | |
| | | protein 5 | | | | |
| Tumor | 202404_s_at | collagen, type I, | COL1A2 | NM_000089 | NM_000089 | Hs.489142 |
| | | alpha 2 | | | | |
| Tumor | 200795_at | SPARC-like 1 | SPARCL1 | NM_004684 | NM_004684 | Hs.62886 |
| | | (mast9, hevin) | | | | |
| Tumor | 214800_x_at | basic transcription | BTF3 | NM_001037637 | R83000 | Hs.591768 |
| | | factor 3 | | /// NM_001207 | | |
| Tumor | 207169_x_at | discoidin domain | DDR1 | NM_001954 /// | NM_001954 | Hs.631988 |
| | | receptor family, | | NM_013993 /// | | |
| | | member 1 | | NM_013994 | | |
| Tumor | 209854_s_at | kallikrein 2, | KLK2 | NM_001002231 | AA595465 | Hs.515560 |
| | | prostatic | | /// | | |
| | | | | NM_001002232 | | |
| | | | | /// NM_005551 | | |
| Stroma | 209854_s_at | kallikrein 2, | KLK2 | NM_001002231 | AA595465 | Hs.515560 |
| | | prostatic | | /// | | |
| | | | | NM_001002232 | | |
| | | | | /// NM_005551 | | |
| Stroma | 200795_at | SPARC-like 1 | SPARCL1 | NM_004684 | NM_004684 | Hs.62886 |
| | | (mast9, hevin) | | | | |
| Stroma | 207169_x_at | discoidin domain | DDR1 | NM_001954 /// | NM_001954 | Hs.631988 |
| | | receptor family, | | NM_013993 /// | | |
| | | member 1 | | NM_013994 | | |
| Stroma | 212647_at | related RAS viral | RRAS | NM_006270 | NM_006270 | Hs.515536 |
| | | (r-ras) oncogene | | | | |
| | | homolog | | | | |
| Stroma | 201131_s_at | cadherin 1, type 1, | CDH1 | NM_004360 | NM_004360 | Hs.461086 |
| | | E-cadherin | | | | |
| | | (epithelial) | | | | |
| Stroma | 214800_x_at | basic transcription | BTF3 | NM_001037637 | R83000 | Hs.591768 |
| | | factor 3 | | /// NM_001207 | | |
| Stroma | 202404_s_at | collagen, type I, | COL1A2 | NM_000089 | NM_000089 | Hs.489142 |
| | | alpha 2 | | | | |
| Stroma | 219960_s_at | ubiquitin carboxyl- | UCHL5 | NM_015984 | NM_015984 | Hs.591458 |
| | | terminal hydrolase | | | | |
| | | L5 | | | | |
| Stroma | 201615_x_at | caldesmon 1 | CALD1 | NM_004342 /// | AI685060 | Hs.490203 |
| | | | | NM_033138 /// | | |
| | | | | NM_033139 /// | | |
| | | | | NM_033140 /// | | |
| | | | | NM_033157 | | |
| Stroma | 205541_s_at | G1 to S phase | GSPT2 | NM_018094 | NM_018094 | Hs.59523 |
| | | transition 2 /// G1 | | | | |
| | | to S phase | | | | |
| | | transition 2 | | | | |
| Stroma | 203084_at | transforming | TGFB1 | NM_000660 | NM_000660 | Hs.155218 |
| | | growth factor, beta | | | | |
| | | 1 (Camurati- | | | | |
| | | Engelmann | | | | |
| | | disease) | | | | |
| Stroma | 207956_x_at | androgen-induced | APRIN | NM_015032 | NM_015928 | Hs.567425 |
| | | proliferation | | | | |
| | | inhibitor | | | | |
| Stroma | 201995_at | exostoses | EXT1 | NM_000127 | NM_000127 | Hs.492618 |
| | | (multiple) 1 | | | | |
| Stroma | 205645_at | RALBP1 | REPS2 | NM_004726 | NM_004726 | Hs.186810 |
| | | associated Eps | | | | |
| | | domain containing 2 | | | | |
| Stroma | 201577_at | non-metastatic | NME1 | NM_000269 /// | NM_000269 | Hs.463456 |
| | | cells 1, protein | | NM_198175 | | |
| | | (NM23A) | | | | |
| | | expressed in | | | | |
| Stroma | 201394_s_at | RNA binding | RBM5 | NM_005778 | U23946 | Hs.439480 |
| | | motif protein 5 | | | | |
| Stroma | 202525_at | protease, serine, 8 | PRSS8 | NM_002773 | NM_002773 | Hs.75799 |
| | | (prostasin) | | | | |
| Stroma | 214460_at | limbic system- | LSAMP | NM_002338 | NM_002338 | Hs.26479 |
| | | associated | | | | |
| | | membrane protein | | | | |
| BPH | 201109_s_at | thrombospondin 1 | THBS1 | NM_003246 | AV726673 | Hs.164226 |
| BPH | 202786_at | serine threonine | STK39 | NM_013233 | NM_013233 | Hs.276271 |
| | | kinase 39 | | | | |
| | | (STE20/SPS1 | | | | |
| | | homolog, yeast) | | | | |
| BPH | 203323_at | caveolin 2 | CAV2 | NM_001233 /// | BF197655 | Hs.212332 |
| | | | | NM_198212 | | |
| BPH | 211945_s_at | integrin, beta 1 | ITGB1 | NM_002211 /// | BG500301 | Hs.429052 |
| | | (fibronectin | | NM_033666 /// | | |
| | | receptor, beta | | NM_033667 /// | | |
| | | polypeptide, | | NM_033668 /// | | |
| | | antigen CD29 | | NM_033669 /// | | |
| | | includes MDF2, | | NM_133376 | | |
| | | MSK12) | | | | |
| BPH | 204470_at | chemokine (C—X—C | CXCL1 | NM_001511 | NM_001511 | Hs.789 |
| | | motif) ligand 1 | | | | |
| | | (melanoma growth | | | | |
| | | stimulating | | | | |
| | | activity, alpha) | | | | |

### Example 5

### Development of Predictive Biomarkers of Prostate Cancer

Cancer gene expression profiling studies often measure bulk tumor samples that contain a wide range of mixtures of multiple cell types. The differences in tissue components add noise to any measurement of expression in tumor cells. Such noise would be reduced by taking tissue percentages into account. However, such information does not exist for most available datasets.

Linear models for predicting tissue components (tumor, stroma, and benign prostatic hyperplasia) using two large public prostate cancer expression microarray datasets whose tissue components were estimated by pathologists (datasets 1 and 2) were developed. Mutual in silico predictions of tissue percentages between datasets 1 and 2 correlated with pathologists' estimates for tumor, stroma and BPH (pairwise comparisons for each tissue p<0.0001). The model from dataset 2 was used to predict tissue percentages of a third large public dataset, for which tissue percentages were unknown. Then datasets 1 and 3 were used to identify candidate recurrence-related genes. The number of concordant recurrence-related markers significantly increased when the predicted tissue components were used. The most significant candidates are listed herein. This is the first known endeavor that finds genes predicative of outcome in two or more independent prostate cancer datasets. Given that tumors are highly heterogeneous and include many irrelevant changes, some markers in adjacent stroma or epithelial tissues could be reliable alternative sensors for recurrent versus non-recurrent cancers. The candidate biomarkers associated with recurrence after prostatectomy are included here.

Previously, a modification of the linear combination model of Stuart et al. 2004 was demonstrated and validated. This method is then employed to correct the independent data to that expected based on cell composition. The corrected data is used to validate genes discovered by analysis of the data to exhibit significant differential expression between non-recurrent and recurrent (aggressive) prostate cancer. The biomarkers of this and previous approaches are compared.

Herein, the result of further manipulation of the data is presented in Table form. A list of genes is provided that cross validate across the U01/SPECS dataset (dataset 1, which has tissue percentage estimated) and the dataset of Stephenson et al. (supra), dataset 3 where tissue percentages are estimated by applying a model based on tissue percentages in Bibilova et al. (supra).

Previous reports summarized efforts toward the development of enhanced methods and specification of genes for the prediction of the outcome of prostate cancer. The current report summarizes continued development of predictive biomarkers of Prostate Cancer.

The goals of this study are to continue development of predicative biomarkers of prostate cancer. In particular the goal of the work summarized here is to use independent datasets to validate genes deduced as predictive based on studies of dataset 1 (infra vide). Here “dataset” refers to the array-based RNA expression data of all cases of a given set together with the clinical data defining whether a given case recurred or remained disease free, a censored quantity. Only the categorical value, recurrent or non recurrent, is used in the analyses described here.

For the purposes of the present work, recurrent prostate cancer is taken as a surrogate of aggressive disease while a non-recurrent patient is taken as indolent disease with a variable degree of indolence that is directly proportional to the disease-free survival time. The dataset 1 contains 26 non-recurrent patients, 29 recurrent patients, the dataset 2 contains 63 non-recurrent patients, 18 recurrent patients, and the dataset 3 contains 29 non-recurrent patients and 42 recurrent patients. The data used for this analysis are subsets of previous datasets. Only samples containing more than 0% tumor and follow-up times longer than 2 years for non-recurrent and 4 years for recurrent cases were included for this particular analysis. The first two datasets' samples have various amount of different tissue and cell types, including tumor cells, stroma cells (a collective term for fibroblasts, myofibroblasts, smooth muscle, and small amounts of nerve and vascular elements), BPH (epithelial cells of benign prostate hypertrophy) and dilated cystic glands (AKA “atrophic” cystic glands), as estimated by four pathologists (Stuart et al., supra) for dataset 1 and one pathologist for dataset 2. Dataset 3 samples were tumor-enriched samples, as claimed by the authors (a coauthor of that study, Steven Goodison, is also a coauthor of Stuart et al. PNAS 2004). In this study, published datasets 2 and 3 were used for the purpose of validation only. A major goal of this study is to use “external” published datasets to validate the properties deduced for genes based on analysis of the dataset 1.

Linear regression analysis was performed on the SPECS (dataset 1) and Goodison (dataset 3) arrays, separately. Estimates of significance of association with recurrence were determined as described in previous updates. The accompanying table filters this data as follows. First, genes associated with recurrence with p<0.1 in any tissue in either dataset were retained. Those genes that showed expression changes that were concordant between datasets were retained. However, the confidence in tissue assignment is not great because stroma and tumor tissue percentages are naturally anti-correlated. Thus, the data was also filtered for genes with p<0.1 which appeared to move in opposite directions in these two tissues across datasets as these are about as likely to be real changes and concordant changes in one tissue across datasets. In addition, genes that had a p<0.01 in one tissue in one dataset were also retained even if the other dataset did not show a significant change, if the fold change in either stroma or tumor was consistent across datasets and there was at least a two-fold change in both datasets. Following these procedures and criteria we observed the results listed in Table 21.

This is the first known endeavor that finds genes predicative of outcome in two or more independent prostate cancer datasets. In addition, some of the identified prognosticators are likely to occur in stroma or in BPH rather than in tumor. Such markers in stroma or BPH may be more easily observed as these tissues are more prevalent and more genetically homogeneous than tumor cells.

TABLE 21
Prognosticators for prostate cancer recurrence after prostatectomy.

(A) Genes predicted to be down regulated in prostate tumor cells
or up regulated in prostate stroma cells in patients in which prostate
cancer will recur after prostatectomy.
(A1) Genes predicted to have expression changes greater than 2-fold
in the current datasets.
201042_at
201169_s_at
201170_s_at
201288_at
201465_s_at
201531_at
201566_x_at
201720_s_at
201721_s_at
202269_x_at
202531_at
202627_s_at
202628_s_at
202643_s_at
203290_at
203932_at
203973_s_at
204070_at
204135_at
204670_x_at
206332_s_at
206360_s_at
206392_s_at
208966_x_at
209138_x_at
209457_at
209823_x_at
210915_x_at
211003_x_at
211430_s_at
211573_x_at
211635_x_at
211637_x_at
211644_x_at
211650_x_at
211798_x_at
213541_s_at
214669_x_at
214768_x_at
214777_at
214836_x_at
214916_x_at
215121_x_at
215193_x_at
(A2) Genes predicted to have expression changes less than
2-fold in the current datasets.
179_at
200748_s_at
200795_at
201367_s_at
201496_x_at
201539_s_at
201540_at
201645_at
201650_at
202205_at
202283_at
202574_s_at
202637_s_at
202748_at
203028_s_at
203052_at
203269_at
203416_at
203591_s_at
203640_at
203748_x_at
203758_at
203760_s_at
203851_at
203923_s_at
204116_at
204192_at
204265_s_at
204438_at
204446_s_at
204561_x_at
204789_at
204790_at
204820_s_at
204890_s_at
204940_at
205375_at
205459_s_at
205476_at
205508_at
205582_s_at
206366_x_at
207201_s_at
207334_s_at
207629_s_at
208110_x_at
208146_s_at
208278_s_at
208461_at
208734_x_at
208889_s_at
209182_s_at
209320_at
209346_s_at
209402_s_at
209447_at
209685_s_at
209873_s_at
209880_s_at
210051_at
210166_at
210190_at
210225_x_at
210298_x_at
210299_s_at
210785_s_at
210845_s_at
210933_s_at
211230_s_at
211628_x_at
211633_x_at
211639_x_at
211649_x_at
211835_at
212016_s_at
212230_at
212613_at
212860_at
212938_at
213095_x_at
213176_s_at
213193_x_at
213293_s_at
213422_s_at
213497_at
213556_at
213958_at
214040_s_at
214219_x_at
214252_s_at
214326_x_at
214450_at
214551_s_at
214567_s_at
215116_s_at
215388_s_at
216224_s_at
216248_s_at
216984_x_at
217227_x_at
217236_x_at
217239_x_at
217326_x_at
217360_x_at
217384_x_at
217478_s_at
217691_x_at
217883_at
218047_at
218087_s_at
218232_at
218301_at
218368_s_at
218718_at
218965_s_at
219202_at
219256_s_at
219541_at
219677_at
221237_s_at
221293_s_at
221667_s_at
221882_s_at
222079_at
222100_at
222210_at
(B) Genes predicted to be up regulated in prostate tumor cells
or down regulated in prostate stroma cells in patients in which
prostate cancer will recur after prostatectomy.
(B1) Genes predicted to have expression changes greater than
2-fold in the current datasets.
201660_at
201661_s_at
201824_at
203791_at
205311_at
205489_at
205860_x_at
211303_x_at
213331_s_at
213510_x_at
214109_at
215363_x_at
217483_at
217487_x_at
217566_s_at
217894_at
217900_at
218224_at
218518_at
218519_at
218930_s_at
219368_at
219685_at
220724_at
221802_s_at
(B2) Genes predicted to have expression changes less than
2-fold in the current datasets.
201782_s_at
202053_s_at
202056_at
202070_s_at
202322_s_at
202337_at
202352_s_at
202538_s_at
202592_at
202596_at
202892_at
202903_at
202919_at
202959_at
203207_s_at
203359_s_at
203503_s_at
203531_at
203538_at
203667_at
203814_s_at
203869_at
204045_at
204159_at
204173_at
204496_at
204554_at
205005_s_at
205055_at
205107_s_at
205160_at
205161_s_at
205303_at
205371_s_at
205565_s_at
205609_at
205830_at
205953_at
205955_at
206571_s_at
206587_at
206920_s_at
206973_at
207071_s_at
207628_s_at
207747_s_at
207769_s_at
208281_x_at
208839_s_at
208873_s_at
208942_s_at
209111_at
209162_s_at
209274_s_at
209585_s_at
209662_at
209817_at
210988_s_at
212208_at
212530_at
212652_s_at
213026_at
213031_s_at
213217_at
213555_at
213701_at
213794_s_at
213893_x_at
214455_at
214527_s_at
214811_at
215412_x_at
216105_x_at
216308_x_at
217645_at
217775_s_at
218009_s_at
218085_at
218197_s_at
218230_at
218260_at
218291_at
218296_x_at
218333_at
218344_s_at
218373_at
218403_at
218499_at
218510_x_at
218521_s_at
218532_s_at
218583_s_at
218633_x_at
218896_s_at
218962_s_at
219007_at
219038_at
219174_at
219206_x_at
219451_at
219467_at
219833_s_at
219997_s_at
220094_s_at
220606_s_at
221265_s_at
221559_s_at
221826_at
222011_s_at
222081_at
47530_at
(C) Genes predicted to be down regulated in benign prostatic
hyperplasia in patients in which prostate cancer will recur after
prostatectomy.
(C1) Genes predicted to have expression changes greater than
2-fold in the current datasets.
200924_s_at
201418_s_at
202415_s_at
203421_at
203577_at
203590_at
204282_s_at
204775_at
206328_at
206866_at
206894_at
206964_at
207631_at
207769_s_at
208141_s_at
210128_s_at
210678_s_at
211512_s_at
212389_at
214311_at
214316_x_at
214819_at
216397_s_at
217264_s_at
217660_at
218372_at
218778_x_at
218965_s_at
219082_at
220388_at
220562_at
221141_x_at
222080_s_at
(C2) Genes predicted to have expression changes less than
2-fold in the current datasets.
200051_at
201640_x_at
202159_at
203128_at
203162_s_at
203321_s_at
206109_at
207484_s_at
207896_s_at
208110_x_at
208278_s_at
208906_at
209202_s_at
209927_s_at
212127_at
212292_at
212456_at
212931_at
213057_at
214778_at
216199_s_at
217468_at
218144_s_at
218744_s_at
219111_s_at
219379_x_at
219986_s_at
221418_s_at
221525_at
221800_s_at
34260_at
(D) Genes predicted to be up regulated in benign prostatic
hyperplasia in patients in which prostate cancer will recur
after prostatectomy.
(D1) Genes predicted to have expression changes greater than
2-fold in the current datasets.
200795_at
201304_at
201435_s_at
201554_x_at
201617_x_at
201745_at
202118_s_at
202437_s_at
202538_s_at
203065_s_at
203224_at
203640_at
204045_at
204438_at
204725_s_at
204940_at
205105_at
205549_at
205609_at
206434_at
208800_at
208839_s_at
208884_s_at
208924_at
209274_s_at
209362_at
209406_at
210299_s_at
210986_s_at
210987_x_at
211562_s_at
211749_s_at
212698_s_at
213325_at
214455_at
216304_x_at
218718_at
218730_s_at
218962_s_at
219410_at
219685_at
219902_at
222150_s_at
222209_s_at
(D2) Genes predicted to have expression changes less than
2-fold in the current datasets.
201133_s_at
201447_at
201448_at
201865_x_at
202056_at
202265_at
202442_at
202666_s_at
202918_s_at
202919_at
203225_s_at
203544_s_at
203562_at
204496_at
205140_at
205659_at
207483_s_at
208290_s_at
208767_s_at
208925_at
209821_at
209882_at
210371_s_at
211727_s_at
211760_s_at
212112_s_at
212397_at
212408_at
212530_at
212607_at
212652_s_at
213102_at
213168_at
213374_x_at
213988_s_at
214686_at
215171_s_at
216115_at
217900_at
218209_s_at
218583_s_at
218729_at
218989_x_at
219230_at
219292_at
221553_at

### Example 6

### Development of Predictive Biomarkers of Prostate Cancer

Datasets Used in this Study

The two datasets used for this study include 1) 148 Affymetrix U133A arrays from 91 patients we acquired (publicly available in the GEO database as accession no. GSE8218, not otherwise published, also referred to as “our data”) which is the principal data set utilized in previous studies; 2) Illumina (of Illumina Inc., San Diego) beads arrays data from 103 patients as analyzed on 115 arrays, a published data set (Bibikova et al., supra);

The two datasets samples have various amount of different tissue and cell types, including tumor cells, stroma cells (a collective term for fibroblasts, myofibroblasts, smooth muscle, and small amounts of nerve and vascular elements), BPH (epithelial cells of benign prostate hypertrophy) and dilated cystic glands (AKA “atrophic” cystic glands), as estimated by four pathologists (Stuart et al., supra) for dataset 1 and one pathologist for dataset 2.

### Determination of Cell Specific Gene Expression in Prostate Cancer

Linear models (Model 1˜3, below) were applied to microarray data from prostate tissues with various amounts of different cell types as estimated by a team of four pathologists. We identified genes specifically expressed in different cell types (tumor, stroma, BPH and dilated cystic glands) of prostate tissue following our published methods (Stuart et al. 2003).

### Model 1˜3:

Cell composition can also be considered as two different cell types; one specific cell type versus all the other cell types, grouped together.

Gᵢ=(βₜᵤₘₒᵣ·Pₜᵤₘₒᵣ+βₙₒₙ₋ₜᵤₘₒᵣ·Pₙₒₙ₋ₜᵤₘₒᵣ)ᵢ

Gᵢ=(βₛₜᵣₒₘₐ·Pₛₜᵣₒₘₐ+βₙₒₙ₋ₛₜᵣₒₘₐ·Pₙₒₙ₋ₛₜᵣₒₘₐ)ᵢ

Gᵢ=(β_{BPH}·P_{BPH}+β_{non-BPH}·P_{non-BPH})ᵢ

The correlation (between probe hybridization intensity and tissue percentages) parameters, such as intercept, slope, probability, standard error, was developed for all the genes on the array from model 1, 2 and 3 using dataset 1 and dataset 2.

### A New Method for the Determination of Cell Type Composition Prediction Using Gene Expression Profiles

Using linear models 1-3, the approximate percents of cell types in samples hybridized to the array may be estimated using only the microarray data based on a sub-list of genes on the array. For example, each gene employed in Model 1 provides an estimate of percent tumor cell composition. We used the median of the predictions based on multiple genes for each tissue type. In our case, only a very limited number of the best tissue-specific genes (5˜41 genes) were used for the prediction. Even fewer genes might be sufficient.

In order to validate the method of tumor or stroma percent composition determination, we utilized the known percent composition figures of data set 1 to predict the tumor cell and stroma cell compositions for data set 2 with known cell composition. For example, the number of genes used for cell type (tumor epithelial cells, stroma cells or BPH epithelial cells) prediction between dataset 1 and dataset 2 ranges from 5 to 41 non-redundant genes, which are listed in Table 20 herein. The Pearson correlation coefficient between predicted cell type percentage (tumor epithelial cells, stroma cells or BPH epithelial cells) and pathologist estimated percentage ranges from 0.45˜0.87.

Since dataset 1 and dataset 2 data were based on different array platforms, the cross-platform normalization were applied using median rank scores (MRS) method (Warnat et al., supra).

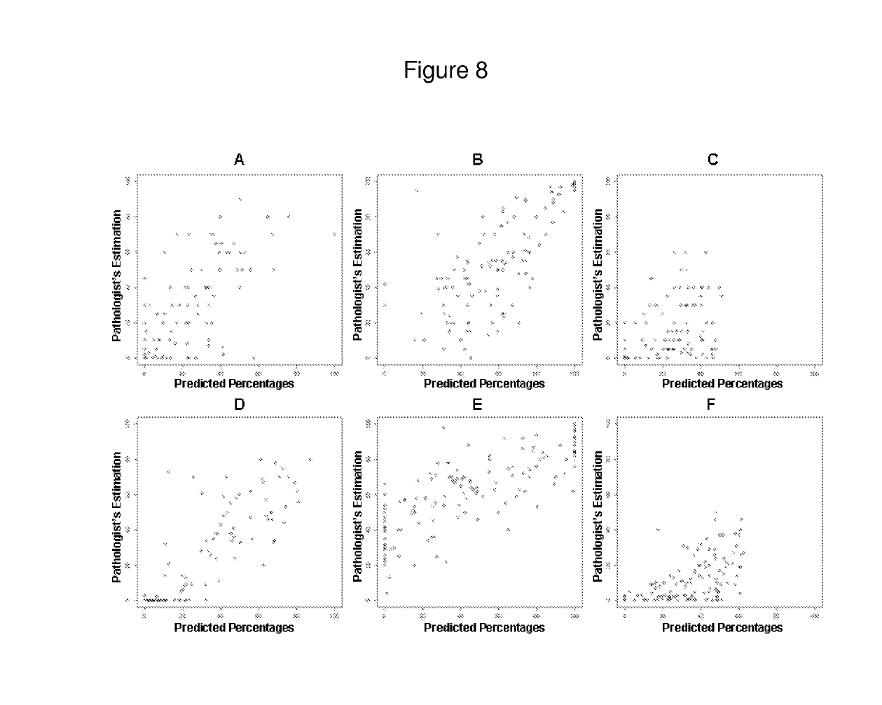

The method of deducing cell type percentage from array data of whole prostate tissue as illustrated here is claimed as novel. FIGS. 8A, 4B and 4C illustrate the use of the parameters of data set 1 to predict the cell composition of data set 2. The Pearson correlation coefficients for the correlation of the observed and calculated cell type compositions is 0.74, 0.70 and 0.45 respectively. The converse calculations of utilizing the parameters of data set 2 to calculate the tumor and stroma cell percent compositions of data set 1 are shown in FIGS. 8D, 4E and 4F respectively, The Pearson Correlation Coefficients are 0.87, 0.78 and 0.57 respectively. The range of Pearson coefficients among four pathologist for composition estimates of the same samples in dataset 1 are 0.92, 0.77 and 0.73 for tumor, stroma and BPH cells respectively (Stuart et al. supra). Thus, the in silico estimates have a correlation that is almost completely subsumed in variation among pathologist, indicating that the in silico estimates are at least similar in performance to a pathologist and leaving open the possibility that the in silico estimates are more accurate than the pathologists.

### Example 7

### Evaluation of Predictive Signatures of Prostate Cancer

Dietary factors have long been considered major factors influencing the development and progression of prostate cancer and Dr. Gordon Saxe of UCSD has published small scale clinical trials showing that diet and life style alterations have a significant impact on the progression of relapsed prostate cancer (Nguyen, Major et al. 2006); (Saxe, Major et al. 2006)). The UCI SPECS study has accepted a “piggy back” project funded by a subcontract from UCSD (G. Saxe, PI) for carrying out a computerized survey of dietary habits of all patients recruited into the SPECS trial at UCI and UCSD. The questionnaire is self administered by providing a laptop computer to postoperative patients and is directly transmitted to Viocare (world wide web at viocare.com), the developers for the questionnaire, where the results are evaluated and provided with comparative statistics for study use. Blood samples are obtained and assessed for carotenoid carotenoids, vitamin D, and other dietary markers (as a validation of reported habits), as well as sex steroid hormones, IG-1, IGFBP-3, and cytokines. Body mass and BMI is measured by standard anthropometry and dexascanning will be introduced shortly to enable more precise evaluation of body composition. The information will be used to independently model diet/nutrition—disease outcome associations and also correlated with our gene expression results to examine diet-gene interactions.

Bioinformatics Identification and Technical Validation of expression biomarkers using Independent test sets of prostate cancer cases. This is focused on the technical and experimental validation of candidate genes that have been identified as differentially expressed in relapsed (aggressive) and non-relapsed (indolent, good prognosis) prostate cancer. Efforts utilized standard approaches such as recursive partitioning (Koziol 2008)PAM, and VSM to identify potential biomarkers. These efforts showed that genes could be defined that preferentially identified cases that relapse early, within two years of prostatectomy, but were not general. This may be due to the heterogeneity of expression in prostate cancer and the need to identify different signatures for different subclasses of prostate cancer, i.e. the development of a true classifier drawn from the appropriate signatures. Efforts have led to significant progress toward this goal. Two factors are particularly significant. First we have made extensive use of multiple linear regression (MLR) analysis first developed by us for analysis of expression of prostate cancer during the predecessor “Director's Challenge” project (Stuart 2004). Second, we have utilized our data set of 147 U133 arrays together with five additional independent data sets of expression data (Table 22). The data sets of Table 22 are a unique resource for validation. The extended MLR approach provides for determining cell-type specific gene expression for four cell types in non-relapsed prostate cancer cases and for the determination of significant changes in expression for the four cell types for relapsed cases, i.e. significantly differentially expressed genes by cell-type in high risk cases. This model is summarized in equation 1:

Gᵢ=β′_{tumor,i}Pₜᵤₘₒᵣ+β′_{stroma,i}Pₛₜᵣₒₘₐ+β′_{BPH,i}P_{BPH}+′_{dilcys gland,i}P_{dilcys gland}+rs(γ_{tumor,i}Pₜᵤₘₒᵣ+γ_{stroma,i}Pₛₜᵣₒₘₐ+γ_{BPH,i}P_{BPH}+γ_{dilcys gland,i}P_{odilcys gland}) (eqn. 1)

where Gᵢ is the observed Affymetrix total Gene expression, the β are the cell-type specific expression coefficients, the P's are the percent of each cell type of the samples applied to the arrays, and the γ's are the differentially expressed component of gene expression for the relapsed cases. When rs=0, no relapse cases are included and the equation is that for gene expression by nonrelapse cases only. The percentages, P, may be determined by examination of H and E slides of the tissue used for RNA preparation by a team of four experienced pathologists. Only two of the six data sets (our cases and those of the Illumina data set, Table 22) have had P's determined by pathologists. Therefore it was first necessary to estimate the percent cell type distribution in all cases of the other four data sets. This was done by using profiles of 40-80 genes for each cell type identified as described (Stuart 2004) that do not vary whether a case is relapse or nonrelapse and are independent of Gleason etc. This method was validated by predicting the percent tumor and stroma cell content of the cases of the Illumina data set which confirmed that the method was accurate (Wang 2007; Wang 2008).

We then applied equation one to our data to identify genes with significant (p<0.01) differential expression in relapsed cases. To validate these genes the process was repeated with each of the five data sets. For each data set we considered a gene as validated if (1) the γ again exhibited p<0.01, (2) were represented by identical Affymetrix probe sets or mapped probe set, and (3) exhibited the same direction change in differential expression. For the tumor cells and stroma cell probe sets, the magnitude of differential expression (the γ) of the two data sets are highly correlated (rₚₑₐᵣₛₒₙ>0.7). Approximately 1000 probe sets were identified that were validated in our data set and one other data set. The number of genes validated in this way is highly significantly greater than the number that may be expected to meet the validation criteria for two data sets by chance. These probe sets represent approximately 693 unique genes owing to a number of genes that were validated in two or more pairs of data sets. Numerous genes correspond to those previously reported by others as related to outcome in prostate cancer and these and many others are functionally related to processes thought important in the progression of prostate cancer. For example several members of the Wnt signal transduction pathway are apparent and are being examined using the TMA.

Discussion. The statistical and biochemical properties of many of these genes support the conclusion that an important signature of outcome for prostate cancer has been obtained. We believe that this is the first use of multiple independent data sets for the validation of signatures of outcome for prostate cancer. Not all validated genes exhibit significant differential expression on all data sets. This provides a picture of the diversity of expression of genes as they appear in independent data sets. Thus, it is possible to construct a true classifier that represents the diversity of all six data sets and this effort is underway. The recognition of diversity among published data sets by a consistent set of criteria provides an explanation for the difficulty of finding a signature based on analyses of one or two data sets.

Experimental validation. As originally proposed, archived prostate cancer cases of the predecessor “Director's Challenge” program that have not been examined by expression analysis are being measured using the U133 plus 2 platform. These cases were recruited in the period 2000-2004. Approximately 25% of these cases have exhibited evidence of relapse. Thus, these cases provide additional valuable material for validating the predictive properties of the recently developed classifiers. The candidate biomarker genes and their ability to function in classifiers identified above will be tested by comparison of the categorization of these new cases with observed survival results. Approximately 300 fresh frozen prostate cancer cases with clinical follow-up have been characterized with respect to tumor content and approximately 80 have sufficient tumor content for analysis. The percent cell-type distribution has been determined by one pathologist and will be refined by use of the four pathologist analysis. Nearly all cases analyzed have yielded excellent RNA and to date 63 cases have been applied to U133 plus 2 arrays and 27 of these cases also have been applied to EXON arrays. Purified RNA and DNA have been banked from all of these cases and may be used, for example, for PCR validation. The analyzed cases were chosen to (2) maximize tumor content and (2) to be approximately equally divided among relapse and nonrelapse cases in order to maximize statistical power for the testing of differential expression. Owing to these criteria, only 15-20 additional cases from the set of 300 will be useful.

The goal of this set of studies is to identify SNP variations and to determine whether particular SNPs correlate with gene expression changes. The potential significance of this study is that SNP sequence maybe determined for any patient from somatic cells such a blood cells or buccal smears. Thus SNP changes that are found to correlate with predictive expression changes may provide to a much more versatile predictive assay. Moreover this information may provide an understanding of the basis of the of the differential expression changes in terms of the properties of location of the correlated SNP.

The platform that is being utilized by D. Duggan is the Illumina one million SNP array and technology. This is the largest coverage array available and provides for sampling of >1 million SNP sequences. The arrays focus on SNP sites near known genes. Over half of all sampled SNPs are within 10 Kb of a gene.

Twenty one nontumor samples from tumor-bearing prostates have been provided and have now been examined on the Illumina platform. These samples are taken from the same 300-case validation set being analyzed by U133 plus 2 and Exon arrays. Approximately equal numbers of know relapse and nonrelapse cases have been provided. All cases have been used to prepare both RNA and DNA. The RNA is archived while the DNA has been applied to the Illumina platform. All cases analyzed have yielded over 90% present calls indicating excellent DNA qc. The data from these first 42 samples will be used for an interim analysis. Owing to the open ended nature of correlating all differentially expressed genes with multiple SNPs, power of the analysis increases with sample numbers and the current plan is to utilize all samples provided to U133 plus 2 arrays to the SNP analysis included relapse and nonrelapse cases.

Tissue microarray development. The goal is to fabricate prostate cancer TMAs to (1) validate newly identified biomarkers, (2) to validate cell-type specific express on the protein level, and (3) to identify antibody reagents for prognostic assay development. To date 494 prostate cancer cases have been provided and 254 have been used for TMA fabrication (Table 23). The major criterion for the selection of cases is that >5 years of survival data be available (except for normal prostate controls) and most of the cases from UCI and LBVA (Long Beach Veterans Administration Medical Center, an associated hospital of the UCI SOM) have 10-19 years of survival data. The original clinical slides of all cases are examined by two pathologists (P. Carpenter and J. Wang-Rodriquez) who regrade Gleason scores and color-encircle zones for core punching. Cores are taken to represent tumor, BPH, tumor-adjacent stroma, far stroma, dilated cystic glands and, where applicable, PIN. TMA fabrication is carried out at the Burnham Institute for Medical Research (S. Krajewski and J. Reed), All chosen fields are represented by two cores. Thus typically each case is represented by 5×2=10 cores. To date 254 cases array contains ˜4000 cores. The four cell types are placed on separate slide arrays so that specialized studies of one cell type do not needlessly consume material. The 494 cases that have been collected for the TMA are entirely independent of all other cases of this study. For approximately two dozen “Director's Challenge” cases that have been used for U133 plus 2 expression analysis there is FFPE tissue which will be applied to the TMA as a means of directly comparing RNA expression and IHC results.

In addition to multiple cell types, several unique features are being developed. Normal prostate control tissue is being incorporated to represent the same cell types as for the cancer cases. These are provided by Sun Health Research Institute (T. Beach and J. Rodgers) based on their rapid autopsy program. These cases are carefully vetted by two pathologists (P. Carpenter and J. Wang-Rodriquez). In addition the time from death to freezing for all cases is recorded and averages 4.25 h for all 65 cases acquired so far but 3.9 h for the cases of the last year. As a further assessment of quality, RNA has been assessed using the Agilent Bioanalyzer for 38 cases (Y. Wang and H. Yao) which indicates intact RNA in 80% of cases and degraded RNA in 10% of cases. Thus, these normal prostates promise to provide an extensive and approximately age-appropriate control panel. A small number of cases contain prostate cancer and may provide an opportunity to determine protein expression differences between clinical and occult disease.

Another unique feature of the TMAs is the collaborative development of quantization being carried out between the BIMR and Aperio Biotechnologies of San Marcos, Calif. This system provides very high resolution line scanning which is stored on a devoted server at BIMR. Specialized software allows retrieval of high power images of any field for remote viewing by participating pathologists via a secure web-based portal (Scancope). Thus finished TMAs are being examined by two pathologists to determine that selected cores indeed represent the Gleason pattern and cell type intended. Moreover, the software provides a database for the survival data associated with each case. Algorithms have been developed by Allen Olson and colleagues of Aperio for the separation of two colors of TMAs labeled with two antibodies developed with different chromagens. In this method a standard antibody that identifies tumor such a AMACR is used for IHC in parallel with a test antibody (second color). Only pixels of the test antibody labeling that colocalizes with AMACR are then selected for correlation with survival data. An example of two color separation using our TMA was published recently (Krajewska, Olson et al. 2007). Quantification is in advanced stages of development.

Numerous antibodies have been screened for use on FFPE sections and 36 have been optimized, applied to one or more of the TMA slides, and digitized as summarized in Table 24. Several antibodies with known behavior in prostate cancer (anti-PSMA, AMACR, E-Cadherin, beta-Catenin, etc.) have been chosen to characterize the arrays while others (anti-Frzd7. SFRP1, PAP, ANX2, etc.) correspond to predicative biomarkers of this study. A number of apoptosis related biomarkers have be identified and the use of BCL-B as a biomarker in prostate and other epithelial tumors has been published recently (Krajewska 2008; Krajewska 2008b).

It is planned to (1) emphasize visual and electronic scoring of the IHC-labeled TMA, (2) validate electronic scoring and (3) evaluate the relationship of antibody labeling and outcome parameters using the Cox-proportional hazard analysis of Kaplan-Meier plots. A second priority will be to continue to expand the TMA to the full 594 case array.

Prognostic test of predicative gene profiles. The goal is to recruit new prostate cancer cases and utilize fresh surgical specimens and biopsies to assess outcome using the current predictive gene profile and to prospectively compare the predicted outcome to observed outcome during year five and as a follow-on long term project. Cases for this study are being recruited in four centers: NWU, UCI, UCSD (SDVA and Thornton Hospitals), and SKCC (Kaiser Permanent Hospital, San Diego). In addition, plans are underway to add the UCI-associated hospital in Long Beach, LBVA. The total number of cases recruited over the past year and from the inception of the study is summarized in Table 25 and associated Demographic, Grading, and Staging data is summarized in Tables 26 and 27. Nearly 1500 cases have been recruited by informed consent to date, over 1300 frozen tissues obtained of which approximately 520 contain tumor. The original goal is to validate selected biomarkers by PCR. Should array costs continue to decrease it may be possible to carryout complete pangenomic expression analysis. By present RNA requirements, conservatively 260 samples would support this effort. Many of these cases have provided blood and post-DRE urine specimens (Table 25) as a further basis for the determination of biomarker expression in more accessible fluids. Shadow charts with baseline data and follow-up data are being developed for all cases.

Diet SPECS study. Patients being recruited for the prostate cancer prospective are being consented to participate in the “piggy back” SPECS diet survey study. To date 27 cases have been consented of which 21 have had blood drawn and provided to the NIH-sponsored General Clinical Research Centers of USCD and UCI (Table 28). In addition 8 patients have completed the computerized questionnaire (Table 28). It is the planned to extend the UCI study to include a second clinic of Dr. D. Ornstein at UCI in addition to the present clinic of A. Ahlering and to continue to enroll all future patients that will be recruited for the prospective study at UCI and UCSD over the coming year. A longer range goal of this study is to utilize the present observational study as a proof of principle that sample acquisition and data base resources are available for the development of a potential phase II trial in which relapsed patients may be offered participation in a randomized intervention trial to test the efficacy of diet and life style change to modify the subsequent course of disease. This initiative will require the development of a new proposal for follow-on funding to the SPECS study.

### REFERENCES

Bibikova, M., E. Chudin, et al. (2007). “Expression signatures that correlated with Gleason score and relapse in prostate cancer.” Genomics 89(6): 666-72.
Koziol, J., Jia, Zhenyu, and Mercola, Dan (2008). “The Wisdom of the Commons: Ensemble Tree Classifiers for Prostate Cancer Prognosis.” Biofinformatics (in revision).
Krajewska, M., Jane N. Winter, Daina Variakojis, Alan Lichtenstein, Dayong Zhai, Michael Cuddy, Xianshu Huang, Frederic Luciano, Cheryl H. Baker, Hoguen Kim, Eunah Shin, Susan Kennedy, Allen H. Olson, Andrzej Badzio, Jacek Jassem, Ivo Meinhold-Heerlein, Michael J. Duffy, Aaron D. Schimmer, Ming Tsao, Ewan Brown, Dan Mercola, Stan Krajewski, John C. Reed. (2008). “Bel-B expression in human epithelial and non-epithelial malignancies.” Proceedings of the 99th Annual Meeting of the American Association for Cancer Research; 2008 Apr. 12-16; San Diego, Calif. (abstract no. 2180.).
Krajewska, M., A. H. Olson, et al. (2007). “Claudin-1 immunohistochemistry for distinguishing malignant from benign epithelial lesions of prostate.” Prostate 67(9): 907-10.
Krajewska, M., Shinichi Kitada, Jane N. Winter, Daina Variakojis, Alan Lichtenstein, Dayong Zhai, Michael Cuddy, Xianshu Huang, Frederic Luciano, Cheryl H. Baker, Hoguen Kim6, Eunah Shin, Susan Kennedy, Allen H. Olson, Andrzej Badzio, Jacek Jassem, Ivo Meinhold-Heerlein, Michael J. Duffy, Aaron D. Schimmer, Ming Tsao3, Ewan Brown, Anne Sawyers, Michael Andreeff, Dan Mercola, Stan Krajewski and John C. (2008b). Reed. Bcl-B Expression in Human Epithelial and Nonepithelial Malignancies Clinical Cancer Research 14, 14: 3011-3021.
LaTulippe, E., J. Satagopan, et al. (2002). “Comprehensive gene expression analysis of prostate cancer reveals distinct transcriptional programs associated with metastatic disease.” Cancer Res 62(15): 4499-506.
Nguyen, J. Y., J. M. Major, et al. (2006). “Adoption of a plant-based diet by patients with recurrent prostate cancer.” Integr Cancer Ther 5(3): 214-23.
Saxe, G. A., J. M. Major, et al. (2006). “Potential attenuation of disease progression in recurrent prostate cancer with plant-based diet and stress reduction.” Integr Cancer Ther 5(3): 206-13.
Singh, D., P. G. Febbo, et al. (2002). “Gene expression correlates of clinical prostate cancer behavior.” Cancer Cell 1(2): 203-9.
Stephenson, A. J., A. Smith, et al. (2005). “Integration of gene expression profiling and clinical variables to predict prostate carcinoma recurrence after radical prostatectomy.” Cancer 104(2): 290-8.
Stuart, R. 0., W. Wachsman, et al. (2004). “In silico dissection of cell-type-associated patterns of gene expression in prostate cancer.” Proc Natl Acad Sci USA 101(2): 615-20.
Wang, Y., Zhenyu Jia, Michael McClelland, and Dan Mercola. (2008). “In silico estimates of tissue percentage improve cross-validation of potential relapse biomarkers in prostate cancer and adjacent stroma.” Proceedings of the 99th Annual Meeting of the American Association for Cancer Research; 2008 Apr. 12-16; San Diego, Calif. (abstract no. 999.).
Wang, Y. K., James; Goodison, Steve; JainJua, Yu, Mercola, Dan, McClelland, Michael. (2007). “Toward the development of a predicative signature of prostate cancer.” Proceedings of the American Association of Cancer Research, Annual Meeting 2007.
Yu, Y. P., D. Landsittel, et al. (2004). “Gene expression alterations in prostate cancer predicting tumor aggression and preceding development of malignancy.” J Clin Oncol 22(14): 2790-9.

The goal of these studies remains the development of a multigene profile that identifies at the time of diagnosis, prostate cancer patients with poor prognosis and good prognosis. Biomarkers have been identified that are validated in at least one independent data set of six data sets available. Moreover the biomarkers represent the diversity of expression among independent data sets. Thus, a true classifier may be formed for the prognosis of prostate cancer.

Current biomarker information is be utilized to develop a test based on the use of FFPE patient tissue, a widely available resource, that may provide improved guidance for prostate cancer patients.

A 254-case TMA is being used to validate selected biomarkers at the protein expression level. The TMA is composed of cases that are independent of the cases utilized to define the biomarkers. Antibodies that perform well may be useful reagents for the development of an IHC-based assay for determining outcome using FFPE prostatectomy tissue or using preoperative biopsy tissue.

Pangenomic expression data has been collected on 60 cases archived from the “Director's Challenge” program and 25 of these cases have also been profiled on the Illumina million SNP chip. This analysis will continue and when suitable numbers are available, SNP alterations that correlate with expression changes will be determined in order that blood cells may provide a means to determine susceptibility to expression of genes associated with behavior to define SNPs with predictive properties. SNPs can be assessed from any tissue, buccal smears or prostate cancer. Patients that are reliably recognized as belonging to either of these groups will be provided with increased knowledge of the likely outcome of their disease and, therefore, may opt for a wider and more appropriate spectrum of treatment.

Patients are being recruited for prospective testing. In addition, certain dietary features are being determined by questionnaire and blood analysis. Patient of this cohort that relapse but do not seek immediate hormonal or radiation therapy may be offered a diet-life style intervention trial. In particular, the over use of radical prostatectomy may be reduced at considerably decreased morbidity, anguish, and expense.

A variety of efforts have been initiated to translate the results into practical tests. High throughput gene expression analysis will allow us to use all 1000 probe sets that we have determined have predictive value to assess risk and compare the assessment to the clinical indicators of risk such as preop PSA, Gleason, and stage and well as outcome over the next few years. Strong indications of predictive value will indicate that biopsy samples should routinely be made available in the fresh state for RNA analysis and provide preoperative information about patients at high risk of disease that may not be cured by surgery and may provide guidance of who would profit from adjuvant therapy. Finally, patients that relapse following surgery commonly have slowly rising PSA values (low PSA doubling time) and many specialists do not immediately recommend hormone or radiation treatment. Such cases may be offered a diet regimen. Our current “piggy back” observational diet study may set the frame work for evaluating the role of diet. In addition the gene signature of such patients will be known and correlations may be carried out to assess whether there is a signature predictive of response. Similarly, by correlating the response to treatment with the known gene expression results, other signatures predictive of response-to-therapy may be determined. These possibilities require that our prospective cohort be examined by expression analysis which requires a large number of arrays not provided for in the original proposal. Thus, work with the prospective cohort will require additional funding for continuation of the translation of the SPECS studies and planning needs to focus on this issue.

TABLE 22
Data Sets Utilized for Identification and Validation of Biomarkers of Relapse of
Prostate Cancer Following Prostatectomy

| | | | | | Time to | | | | | |
|---|---|---|---|---|---|---|---|---|---|---|
| | | | | Non- | Relapse | preO | | | | |
| Data | Array | | Relapse | Relapse | data | P- | | TNM | | |
| Sets | platform | Targets^{d} | (total) | (total) | available? | PSA | Gleason | stage | | Ref. |
| 1^{a,b} | U133A2 | 22,283 | 85 | 57 | yes | yes | yes | yes | yes | 1 |
| 2^{a} | Illumina | 511 | 25 | 84 | partial | no | yes | yes | no | 2 |
| | | | | | (only for | | | | | |
| | | | | | relapse | | | | | |
| | | | | | samples) | | | | | |
| 3^{c} | U133A | 22,283 | 37 | 42 | no | yes | yes | yes | no | 3 |
| 4 | U95Av2 | 12,626 | 8 | 13 | no | no | no | no | no | 4 |
| 5 | U95Av2, B, C | 37,891 | 23 | 25 | yes | yes | yes | yes | no | 5 |
| 6 | U95Av2 | 12,626 | 9 | 14 | no | yes | yes | yes | no | 6 |

^{a}Contains data on tissue percentages.
^{b}These data sets contain information on follow-up time. Relapse was defined as PSA reaches detectable level after prostatectomy within the first four years. All non-relapse cases were cases followed-up over two years and showed no sign of relapse.
^{c}These data sets contain information on follow-up time. Relapse was defined as three consecutive PSA increases >0.1 ng/ml within the first four years. All non-relapse cases were cases followed-up over two years and showed no sign of relapse.
^{d}Number of target transcripts represented on the array.
Ref. 1, (Stuart, Wachsman et al. 2004)
Ref. 2, (Bibikova, Chudin et al. 2007)
Ref. 3, (Stephenson, Smith et al. 2005)
Ref. 4, (Singh, Febbo et al. 2002)
Ref. 5, (Yu, Landsittel et al. 2004)
Ref. 6, (LaTulippe, Satagopan et al. 2002)

TABLE 23
UCI SPECS Tissue Microarray (TMA) Development Status

| | Since Inception of Study | year 2 |
|---|---|---|
| Characteristic | | |
| Prostate Cases on the Array | 254 | |
| as of May 01, 2008 | (~1000 cores) | |
| Prostate Cases by Source on or | 494 | 219 |
| available for the Array | | |
| 1. UCI Medical Center Cases | 203 | 95 |
| 2. Long Beach VA Medical | 165 | 90 |
| Center Cases | | |
| 3. SKCC | 66 | |
| 4. Sun Health Res. Inst | 60 | 34 |
| Grade and Stage Distribution | | |
| (UCI/LBVA) | | |
| Gleason 4-7 | 159 | 135 |
| Gleason 8-10 | 26 | 50 |
| High Grade Prostate | 95 | 161 |
| Intraepithelial Neoplasia (PIN) | | |
| Lymph Node Metastasis | 9 | 2 |

TABLE 24
Antibodies applied to the SPECS TMA

| | | | | Digitized | Digitized |
|---|---|---|---|---|---|
| Standardization | | | | Virtual | Virtual |
| Antibody | Type | Antibody | Array ID# | slide | Block |
| AMACR | Rb- | DAKO#M3616 | TMA# 83-84; | yes | TMA# 83- |
| E-Cadhedrin | MAB | BD#610181 | TMA# 83-84; | yes | TMA# 83; 95 |
| PSA | MAB | DAKO | TMA# 83-84; | yes | TMA# 83- |
| PSMA | | no antibody | TMA #83-84; | no | |
| Beta-Catenin | MAB | BD | TMA# 83-84; | yes | TMA# 83- |
| | | Transduction | 94-97 | | 84; 95 |
| | | Lab; #610154 | | | |
| Prostate-Acid | Rb polyclonal | Sigma# P56641 | TMA# 83-84; | yes | TMA# 83- |
| [symbol] | | | [symbol] | | [symbol] |
| SFRP1 | Rb polyclonal | Novus; NB600- | TMA #83-84; | yes | no |
| | | 499 | TMA 94-97 | | |
| FRZD7 | Rb | GenWay 18- | TMA #83-84; | yes | no |
| | polyclonal/Aff | 141-10554 | TMA 94-97 | | |
| | [symbol] | 18-003-42797 | | | |
| Annexin 2 | | | TMA #83 | yes | no |
| IL-6 | Mouse | GenWay 20- | TMA #83-84; | yes | no |
| | [symbol] | [symbol] | [symbol] | | |
| Bnip3 | Rb polyclonal | BIMR/AR-46 | TMA #83-84; | yes | no |
| | | [symbol] | [symbol] | | |
| 14-3-3 zeta, | Rb polyclonal | Abcam 18706 | TMA #83- | yes | no |
| [symbol] | | | [symbol] | | |
| CD46 | Goat antihu | R&D: AF2005 | TMA #83- | yes | no |
| PED/PEA 15 | Rb polyconal | Novus ab 1832 | TMA #83- | yes | no |
| Phosphospecific | | R&D AF 0225 | 84/sub | | |
| PAR4 (R- | Rb polyconal | SC-1807 | TMA #83- | yes | no |
| Cart. | Rat | ABD Serotec; | TMA #83- | yes | no |
| Matrix Prot | antihuman | MCA 1455 | 84/sub | | |
| HIF1-alpha | MAB | Novus, 100123 | TMA #83-84 | yes | no |
| Siah2 (SR) | MAB | Sigma; (Ronai | TMA #83-84 | yes | no |
| | | [symbol] | | | |
| Sip- | Rat | (Ronai Collab) | TMA #83-84 | yes | no |
| | Rab | BIMR/AR-75 | TMA #83-84 | yes | no |
| | | BIMR/AR-75 | TMA #83-84 | yes | no |
| PHD3 | MAB | (Ronai Collab) | TMA #83- | yes | no |
| Claudin 1 | Rb-poly | Zymed#: 51- | TMA# 83-84; | yes | no |
| BclG | Rb polyconal | BIMR AR-120; - | TMA# 83-84; | yes | yes |
| | | 121 | 94-97 | | |
| BclB | Rb polyconal | BIMR/AR-49 | TMA #83-84 | yes | yes |
| PDGF-c | Rb polyconal | Santa Cruz; (c- | TMA #83 | yes | no |
| | | [symbol] | | | |
| DDR1 | Rb polyconal | Collab-China | TMA#83; 94- | yes | No |
| ER-beta | MAB | GeneTex | TMA #83 | yes | Yes |
| BFL1 | Rb | BIMR/BR-50 | TMA #83-84 | yes | Yes |

Pending

| | | | | | |
|---|---|---|---|---|---|
| ELF3 | Mouse | 20-372-60074 | Not tested | no | No |
| ANNEXIN 1 | | | Not tested | no | No |

Double Staining

| | | | | | |
|---|---|---|---|---|---|
| Claudin + Amacr | Rb poly/Mono | | TMA #83-84 | yes | Yes |
| AR&PSA | Rb poly/MAB | Santa Cruz: | TMA# 94-97 | yes | TMA#; 95 |
| | | [symbol] | | | |
| BCL2/TR3 | Rb/MAB | AR- | TMA#83; 94- | yes | TMA# 95 |
| | | 01/R&D#: | 97 | | |
| BAX/HIF1alpha | Rb/MAB | AR-02/Novus: | TMA#83; 94- | yes | TMA# 95 |
| | | NB100-123 | 97 | | |

[symbol]

indicates data missing or illegible when filed

TABLE 25
Summary of samples collected for prospective study during the
current funding period and since the inception of the study.
Interval Summary of Consented SPECS Patients since Jul. 1, 2007

| | SKCC | | UCSD/VAMC- | | |
|---|---|---|---|---|---|
| Characteristic | (KPH) | NWU | SD | UCI | |
| Consented Cases | 45 | 335 | 295 | 85 | |
| BPH | | 9 | 47 | | |
| Prostate Cancer | | 339 | 100 | | |
| Tissues Obtained (frozen) | 40 | 267 | 147 | | |
| Samples with Tumor | 45% | 34 (13%) | | 53 | (62%) |
| Samples without Tumor | 55% | unknown | | 32 | (48%) |

| Sample Review Pending | | 238 | | | 0 |
|---|---|---|---|---|---|
| Mean Sample Tumor % | | 16% | | | |
| Banked Plasma | 40 | 78 | 215 | | 55 |
| Banked Urine | 40 | 78 | 238 | (94 postDRE) | 39 |

Consented SPECS Patients since inception of the study (Sep. 30, 2005)

| | SKCC | | | | | |
|---|---|---|---|---|---|---|
| | (KPH) | NWU¹ | UCSD/VAMC-SD | | UCI | |
| Consented (TOTAL 1489) | 59 | 711 | 404 | | 304 | |
| Mean Age | 60.5 | 62.4 | 64 | (41-85) | 62 | |
| BPH | 0 | 10 | 81 | | | |
| Mean PSA (ng/ml) | | unknown | 2.8 | (<0.15-30.8) | 6.66 overall av | |
| Prostate Cancer | 59 | 274 | 175 | | 213 | |
| Mean PSA (ng/ml) | | 5.6 ± 3.6 | 7.53 | (0.22-77.8) | 6.66 overall av | |
| Tissues Obtained (frozen) | 59 | 572 | 210 | | 420 | |
| Samples with Tumor | | 127 | 30% | | 213 | (51%) |
| Samples without Tumor | | Unknown | 30% | | 145 | (49%) |
| Sample Review Pending | | 466 | 40% | | 0 | |
| Mean Sample Tumor % | | 12.2% | 53% | | | |
| Banked Plasma | 59 | 176 | 317 | | 209 | |
| Banked Urine | 59 | 174 | 339 | (94postDRE) | 174 | (postDRE) |

| | | |
|---|---|---|
| Number/percent NED since surg | 75% | |
| Number/percent chemical | 3% | 0 |
| relapse (PSA > 0.2 ng/ml) | | |
| Number/percent neg postop | 74% | 150 |
| PSA | | |
| Number/percent pos postop PSA | 8% | 3 |
| Number pending PSA | 18% | |

TABLE 26
Ethnicity of Consented Cases for Prospective Analysis

| | UCSD | | | | | | UCI | | NWU | | SKCC | |
|---|---|---|---|---|---|---|---|---|---|---|---|---|
| | n = 181 | | UCSD | | UCSD | | n = 302 | | n = 711 | | n = 59 | |
| | Consented | | n = 140 | | n = 41 | | Consented | | Consented | | Consented | |
| Characteristic | Pts | | PCA | | BPH | | Pts | | Pts | | Pts. | |
| Mean age at | 64 | (41-85) | 62 | | 66 | | 62 | | 62.4 | | 60.5 | (47-73) |
| enrollment | | | | | | | | | | | | |
| Median age at | 63 | (41-85) | 61 | (41-84) | 64 | (54-85) | 62 | | | | 60.0 | (47-73) |
| enrollment | | | | | | | | | | | | |
| Ethnicity | 181 | | 140 | | 41 | | | | | | 59 | |
| African-American | 19 | (10%) | 17 | (12%) | 2 | (5%) | 2 | (0.7%) | 39 | (0.5%) | 2 | (3%) |
| Asian/Pacific | 2 | (1%) | 2 | (1%) | 0 | | 14 | (4.7%) | 4 | (.05%) | 1 | (2%) |
| Islander | | | | | | | | | | | | |
| Caucasian | 139 | (77%) | 105 | (75%) | 35 | (87%) | 184 | (61%) | 579 | (81%) | 19 | (32%) |
| Filipino | 5 | (3%) | 5 | (3.5%) | 0 | | 0 | | unknown | | | |
| Native American | 1 | (<1%) | 1 | (<1%) | 0 | | 0 | | unknown | | | |
| Hispanic | 8 | (4%) | 5 | (3.5%) | 3 | (7.5%) | 1 | (0.03%) | 13 | (1.8%) | 5 | (8%) |
| Hawaiian | 1 | (<1%) | 1 | (<1%) | 0 | | 0 | | n/a | | | |
| Other Ethnicity | 2 | (1%) | 1 | (<1%) | 1 | (2.5%) | 45 | (15%) | n/a | | | |
| Not | 4 | (2%) | 4 | (3%) | 0 | | 56 | (19%) | 76 | (11%) | 32 | (54%) |
| Reported/unknown | | | | | | | | | | | | |
| Subtotals | 181 | | 140 | | 41 | | 302 | | 711 | | 59 | |
| Totals | | | | | | | | | | | 1434 | |

TABLE 27
Gleason Score Distribution and Stage Distribution for
Consented Cases for Prospective Analysis

| | UCSD | NWU | UCI | SKCC |
|---|---|---|---|---|
| GLEASON | | | | |
| 2 + 3 = 5 | 1 | 0 | 1 | 0 |
| 3 + 2 = 5 | 2 | 0 | 1 | 0 |
| 2 + 4 = 6 | 1 | 0 | 0 | 0 |
| 3 + 3 = 6 | 47 | 145 | 80 | 19 |
| 3 + 4 = 7 | 37 | 108 | 123 | 23 |
| 4 + 3 = 7 | 13 | 21 | 49 | 3 |
| 3 + 5 = 8 | 2 | 0 | 2 | 1 |
| 5 + 3 = 8 | 1 | 1 | 0 | 0 |
| 4 + 4 = 8 | 12 | 6 | 7 | 0 |
| 4 + 5 = 9 | 10 | 7 | 13 | 0 |
| 5 + 4 = 9 | 5 | 3 | 0 | 0 |
| 5 + 5 = 10 | 1 | 0 | 0 | 1 |
| | 132 | 291 | 276 | 59 |
| No PCA on Path | 4 | na | 2 | 13 |
| Pathology Pending | 7 | na | 0 | na |
| | 143 | 291 | 278 | 59 |
| STAGE | | | | |
| pT0 | 2 | na | 2 | 0 |
| pT2a | 14 | na | 27 | 3 |
| pT2b | 6 | na | 0 | 0 |
| pT2c | 88 | na | 170 | 35 |
| pT3a | 10 | na | 54 | 5 |
| pT3b | 9 | na | 5 | 3 |
| pt3(a + b) | na | na | 10 | 0 |
| pT2 | na | na | 2 | |
| pT3 | na | na | 4 | |
| pT4 | na | na | 4 | |
| | 129 | | 278 | 43 |
| Channel TURP | 4 | | na | 0 |
| Missing Path Stage | 4 | | na | 13 |
| Pathology Pending | 7 | | na | 0 |
| | 144 | 291 | 278 | 59 |

TABLE 28
Summary of cases consented for the observational diet SPECS study

| | | | | | Scheduled |
|---|---|---|---|---|---|
| | | | Blood | | for |
| | | | to | Questionnaire | home |
| Site | Start | Consented | GCRC | completed | completion |
| UCSD | December | 23 | 18 | 7 | 2 |
| | 2007 | | | | |
| UCI | April 2008 | 18 | 17 | 11 | 7 |
| Total | | 41 | 35 | 18 | 9 |

### The Challenge of Developing Predictive Signatures for the Outcome of Newly Diagnosed Prostate Cancer Based on Expression Analysis and Genetic Changes of Tumor and Non-Tumor Cells

Linear regression analysis was used to determine the average gene expression profile of four cell types, including tumor and stroma cells, in a set of 88 prostatectomy samples (1). By combining these cases with 55 additional cases with Affymetrix U133A gene expression data, we were able to select 63 cases in which disease relapsed over a period of three or more years following prostatectomy. Linear regression analysis of the non-relapse and relapse sets revealed changes in hundreds of gene expression values, including genes primarily expressed in stroma cells that were associated with the relapse status. These genes were used to generate classifiers using two other independent Affymetrix expression datasets generated from enriched prostate tumors. One dataset of 79 samples (37 relapse, Affymetrix U133A array; training-set) was used as the training set (2), and one dataset of 48 samples (23 relapse, Affymetrix U95Av2/U95B/U95C array was used as the test-set (3). Probe sets across platforms were mapped using the Affymetrix array comparison spreadsheet and normalized using quantile discretization (4). Classifier genes were determined by use of recursive partitioning (RP) in which a handful of genes are used sequentially for classification (5), as well as Prediction Analysis of Microarrays (PAM)(6), in which case outcomes were predicted via a nearest shrunken centroid method from gene expression data (1). RP classification trees using up to five genes, and sometimes including pre-operative PSA, routinely classified each independent dataset into three survival groups, non-relapse, early relapse, and late relapse with p<0.005. Classifiers generated by PAM using tumor specific genes predicted by linear regression as input was as good (accuracy, sensitivity, specificity) as the best classifiers using all of the expression data, indicating an enrichment for relevant genes by the linear regression method (SVM was dropped from here since it did not perform better than PAM). However classifier performance decreased with increased disease-free survival of the cases. A 59-gene classifier determined by PAM using all cases of the training set with times-to-relapse of <2 years yielded a specificity of 75.9% and a sensitivity of 88.0% with an overall accuracy of 73.4% when tested with the second independent data set for cases of the same time period. All three performance values decreased continuously upon inclusion of longer time periods to <4 y. No reliable PAM classifiers could be generated for late relapse cases. RP consistently yielded a major group of nonrelapse cases and two classes of relapse cases, one of which consists of very early relapse cases with disease-free survival of <2 years. The distinction of late relapse cases from nonrelapse cases using PAM remains a challenge and may reflect the similarity of gene expression profiles of nonrelapse cases from those destined to relapse relatively late after diagnosis. Prediction of early relapse at the time of diagnosis may be a realistic goal.
1. Stuart, R., et al. PNAS 2004; 201:615-20; 2. Stephenson et al. Cancer. 2005; 104:290-8. 3. Yu Y., et al. J. Clin. Oncol. 2004; 22:1790.4. Warnat, P., et al. BMC Bioinformatics. 2005; 6:265. 5. Koziol, J., et al. Cancer Res. 2003; 9:5120-6. 6. Tibshirani, R. et al. PNAS 2002; 99:6567-72.

### A New Bi-Model Approach for the Development of a Classifier for Predicting Outcomes of Prostate Cancer Patients

Prostate cancer is the most common malignancy of males. However, the majority of cases are “indolent” and may not threaten lives. In order to improve disease management, reliable molecular indicators are needed to distinguish the indolent cancer from the cancer that will progress. Statistical methods, such as hierarchical clustering, PAM and SVM, have been widely used for classifier development for various cancers. However, those methods can not be immediately applied to prostate cancer research because the tissue samples collected from patients are very heterogeneous in cell composition. The observed expression level of any gene for a given sample is not solely for tumor cells; rather, it is the sum of contributions from all types of cells within that sample. In current study, we propose a novel method where the expression level of any gene is illustrated with a linear model considering the contributions from different types of cells and their interactions with aggression phases (relapse or non-relapse). ANOVA is used to identify cell specific relapse associated genes that possess discriminative power. The expression patterns of those selected genes may be described using two Gaussian models on the basis of disease phases; thus they can be used for predicting outcomes of newly diagnosed. The new method is compared to other conventional methods based on simulated data. A predictive classifier is created by training a real dataset generated for prostate cancer research. The performance of the new classifier is compared to the nomogram and other clinical parameters with predictive value.

### In Silico Estimates of Tissue Percentage Improve Cross-Validation of Potential Relapse Biomarkers in Prostate Cancer and Adjacent Stroma

Differences in RNA levels that correlated with relapse versus non-relapse were calculated for two public expression microarray data sets using two models. One model did not take into account tumor and stroma tissue percentages in each sample, and the other used these percentages in a linear model. The latter model led to a highly significant increase in the number of candidate relapse-associated biomarkers cross-validated between both data sets. Many of these relapse-associated changes in transcript levels occurred in adjacent stroma. Estimates of tissue percentages based on expression data applied between data sets correlated almost as well as multiple pathologists correlated with each other within a data set. This in silico model to predict tissue percentage was applied to a third public data set, for which no tissue percentages exist. Cross-validation of relapse-associated genes between data sets was again highly significantly improved using the linear model, and included changes in stroma. The third data set was heavily skewed towards a previously unrecognized higher tumor percentage in relapse versus non-relapse cases, a bias that is taken into account by the linear model. In summary, the use of tissue percentages determined by a pathologist or inferred from in silico data increased the power to detect concordant changes associated with a clinical parameter in separate data sets, and assigned these changes to different tissue compartments. The strategy should be applicable for biomarkers other than RNA and for samples from any type of disease that contains measurable mixed tissues.

### Improved Identification of RNA Prognostic Biomarkers for Prostate Cancer Using in Silico Tissue Percentage Estimates

Although many studies of detecting RNA-based prognosticators for prostate cancer have been performed, they have limited agreement with each other. One contributing factor may be the variations in the proportion of tissue components in prostate tissue samples, which leads to considerable noise and even misleading results in mining microarrays data.

We assembled six microarray data sets for RNA expression in prostate cancer samples with associated relapse information, including two large data sets of our own. Our two datasets, and one other, included estimates of tissue percentages made by pathologists. These data sets were used to identify genes that were then used to build a simple linear model for tissue percentage prediction. Estimates of tissue percentages based on expression data applied between data sets correlated almost as well as multiple pathologists correlated with each other within a data set.

Using a multiple linear regression (MLR) model which integrates tissue component percentages, we identified a list of tumor- and reactive stroma-associated prognostic RNA biomarkers in all six data sets. The level of each RNA is expressed as a linear model of contributions from the different cell types and their interactions with relapse status

$$g = {b}_{0} + {\sum }_{j = 1}^{C} {b}_{j} {p}_{j} + RS \times {\sum }_{j = 1}^{C} {γ}_{j} {p}_{j} + e ,$$

where g is expression intensity, C is the number of cell types, RS is relapse status indicator, e is random error, and b's and y's are regression coefficients. ANOVA is used to identify cell specific genes that are differentially expressed between relapsed and non-relapsed cases, i.e., the genes with significant γ's. Markers were then cross-validated between the six different microarray data sets. There were 185 genes that occurred in more than one data set, and 152 of 185 (82.2%) showed the same direction of change in differential expression between relapse and non-relapse patient samples (p<10⁻¹⁸). Most of these prognostic markers were not previously identified by other studies and some were potentially differentially expressed in stroma.

In summary, the use of tissue percentages determined by a pathologist or inferred from in silico data increased the power to detect differential expressed genes associated with a clinical parameter and assigned these changes to different tissue compartments. The strategy should be applicable for biomarkers other than RNA and for samples from any type of disease that contains measurable mixed tissues.

A Bi-Model Classifier that Allows RNA Expression in Mixed Tissues to Be Used in Prostate Cancer Prognosis

Introduction: Reliable molecular indicators are needed to distinguish indolent prostate cancer from cancer that will progress. Statistical methods, such as hierarchical clustering, PAM and SVM, have been widely used to develop classifiers of prognostic molecular markers that estimate risk. However, one barrier to the efficient use of classifiers in prostate cancer is the variable mixture of different cell types in most clinical samples. The observed level of any marker for a given sample is due to the sum of contributions from all types of cells within the tumor. Elsewhere [1], we propose a novel classification method in which the expression level of any gene is expressed as a linear model of contributions from the different cell types and their interactions with relapse status. While this method provides biomarkers with greater confidence by deconvoluting the effect of tissue percentages in each sample, the problem of how to construct a classifier for mixed populations remains.

Methods: We propose that the expression patterns of prognostic RNAs may be described using either of two Gaussian models, one for relapsed cases and the other one for non-relapsed cases, both of which include calculation with cell constitute information. A likelihood-ratio statistic (LR) can be developed by contrasting the probability of being risk free to the probability of undergoing relapse based on fitting expression values of selected biomarkers and the cell composition data of each sample to these two differential models. A patient is diagnosed as having high risk of relapse if LR≧k₁, or is diagnosed as being of low risk if LR≦k₂, where k₁ and k₂ are pre-selected cutoffs with k₁>1>k₂.

Results: In a simulation study, the new method outperformed the conventional classification methods PAM and SVM. A prognostic classifier was then created by training an expression dataset generated from Affymetrix U133P2 arrays from prostatectomies with known tissue composition, which yielded a 50 gene classifier with an accuracy of 94% following cross validation. When the predictive classifier was applied to an independent “test” data set based on 35 Affymetrix U133A arrays, an accuracy of 80% was achieved

Conclusion: This novel classifier may be useful for assessing risk of relapse at the time of diagnosis in clinical samples with variable amounts of cancer tissue.

### REFERENCE

[1] Wang, Y., et al., Proc. 100^{th} Annual meeting of the AACR. [abstract].

### The Prostate Tumor Microenvironment Exhibits Numerous Differentially Expressed Genes Useful for Diagnosis

Introduction: There are over one million prostate biopsies performed in the U.S. annually. Pathology examination misses the tumor entirely in a few percent of cases. In an additional 10-20% of cases the biopsies are not definitive due to atypical foci, PIN, or other caveats, often leading to a “repeat biopsy” in 6-12 months. We observed that the microenvironment of prostate tumor cells exhibits numerous differential gene expression changes compared to remote stroma tissue of the same cases. Such changes could be useful to form a classifier for the diagnosis of prostate cancer when tumor is present in very low amounts or is barely missed by a biopsy.

Methods: A training set of 105 prostate cancer cases was created with known cell type composition for the three major cell types of tumor tissue (tumor epithelial cells, epithelial cells of BPH and stroma cells) as assessed by four pathologists. RNA expression was measured on U133plus2 GeneChips. A linear model defined the total signal as the sum of expression values of the three cell types each weighted by its percent composition figure for a given case:

Gi=βtumorPtumor+βstromaPstroma+βBPHPBPH

where Gi is the fluorescence intensity for a gene of a case, Pi are the percents of the indicated cell type and βi are cell-specific expression coefficients (signal/percent cell type). The model was applied separately to tumor-bearing tissues and tumor-free remote stroma tissues. Differential gene expression was derived by subtraction of the values for the two series.
Results: The ˜200 most significant differences were used as input to PAM. Tenfold cross-validation dichotomized the training set into tumor-bearing and remote stroma tissues, yielding a classifier of 36 genes that had a 94% accuracy. This classifier was then tested using an independent set of 82 cases, as well as 13 control normal prostate stroma tissues. The classifier had an accuracy of 83% on the test set. Correct classification was also achieved for five of six biopsies from normal males and all seven cases from the rapid autopsy. Several genes such as myosin VI, collagen IX, and destrin, known to be highly expressed in mesenchymal derivatives, are preferentially expressed in tumor-adjacent stroma.
Conclusions: The differential gene expression changes observed here most likely represent differences in expression between tumor-adjacent stroma and remote stroma. These differences may be due to paracrine or “field effect” mechanisms involving interaction with the tumor adjacent to the affected stroma. The reaction of stroma to nearby prostate cancer is well-known but, as observed here, involves many more gene changes than previously recognized. These changes can be exploited to develop a classifier that accurately categorizes tumor-bearing tissues, remote tissues of the same cases and normal tissues. Such a classifier could enhance diagnosis from false negative and equivocal biopsy results.

TABLE 29
125 Genes generated by one of the two methods for identifying reactive stroma genes

| Probe.Set.ID | Gene.Title | Gene.Symbol |
|---|---|---|
| 204934_s_at | hepsin (transmembrane protease, serine 1) | HPN |
| 209426_s_at | alpha-methylacyl-CoA racemase /// C1q and tumor | AMACR /// C1QTNF3 |
| | necrosis factor related protein 3 | |
| 64486_at | coronin, actin binding protein, 1B | CORO1B |
| 203755_at | BUB1 budding uninhibited by benzimidazoles 1 | BUB1B |
| | homolog beta (yeast) | |
| 203317_at | pleckstrin and Sec7 domain containing 4 | PSD4 |
| 211576_s_at | solute carrier family 19 (folate transporter), member 1 | SLC19A1 |
| 202148_s_at | pyrroline-5-carboxylate reductase 1 | PYCR1 |
| 205339_at | SCL/TAL1 interrupting locus | STIL |
| 211984_at | calmodulin 1 (phosphorylase kinase, delta) /// | CALM1 /// CALM2 /// |
| | calmodulin 2 (phosphorylase kinase, delta) /// | CALM3 |
| | calmodulin 3 (phosphorylase kinase, delta) | |
| 217912_at | dihydrouridine synthase 1-like (S. cerevisiae) | DUS1L |
| 218275_at | solute carrier family 25 (mitochondrial carrier; | SLC25A10 |
| | dicarboxylate transporter), member 10 | |
| 202645_s_at | multiple endocrine neoplasia I | MEN1 |
| 209424_s_at | alpha-methylacyl-CoA racemase /// C1q and tumor | AMACR /// C1QTNF3 |
| | necrosis factor related protein 3 | |
| 206558_at | single-minded homolog 2 (Drosophila) | SIM2 |
| 219360_s_at | transient receptor potential cation channel, subfamily | TRPM4 |
| | M, member 4 | |
| 220584_at | hypothetical protein FLJ22184 | FLJ22184 |
| 201420_s_at | WD repeat domain 77 | WDR77 |
| 218683_at | polypyrimidine tract binding protein 2 | PTBP2 |
| 208190_s_at | lipolysis stimulated lipoprotein receptor | LSR |
| 219809_at | WD repeat domain 55 | WDR55 |
| 219395_at | RNA binding motif protein 35B | RBM35B |
| 207239_s_at | PCTAIRE protein kinase 1 | PCTK1 |
| 218180_s_at | EPS8-like 2 | EPS8L2 |
| 203287_at | ladinin 1 | LAD1 |
| 33814_at | p21(CDKN1A)-activated kinase 4 | PAK4 |
| 218365_s_at | aspartyl-tRNA synthetase 2, mitochondrial | DARS2 |
| 208824_x_at | PCTAIRE protein kinase 1 | PCTK1 |
| 219148_at | PDZ binding kinase | PBK |
| 201819_at | scavenger receptor class B, member 1 | SCARB1 |
| 218874_s_at | chromosome 6 open reading frame 134 | C6orf134 |
| 204532_x_at | UDP glucuronosyltransferase 1 family, polypeptide | UGT1A1 /// |
| | A10 /// UDP glucuronosyltransferase 1 family, | UGT1A10 /// |
| | polypeptide A8 /// UDP glucuronosyltransferase 1 | UGT1A4 /// UGT1A6 |
| | family, polypeptide A6 /// UDP | /// UGT1A8 /// |
| | glucuronosyltransferase 1 family, polypeptide A9 /// | UGT1A9 |
| | UDP glucuronosyltransferase 1 family, polypeptide | |
| | A4 /// UDP glucuronosyltransferase 1 family, | |
| | polypeptide A1 | |
| 217099_s_at | gem (nuclear organelle) associated protein 4 | GEMIN4 |
| 214393_at | Rho family GTPase 2 | RND2 |
| 204714_s_at | coagulation factor V (proaccelerin, labile factor) | F5 |
| 209972_s_at | JTV1 gene | JTV1 |
| 213464_at | SHC (Src homology 2 domain containing) | SHC2 |
| | transforming protein 2 | |
| 221665_s_at | EPS8-like 1 | EPS8L1 |
| 202740_at | aminoacylase 1 | ACY1 |
| 209015_s_at | DnaJ (Hsp40) homolog, subfamily B, member 6 | DNAJB6 |
| 200678_x_at | granulin | GRN |
| 210480_s_at | myosin VI | MYO6 |
| 220354_at | similar to hCG1774568 | LOC100134018 |
| 210627_s_at | glucosidase I | GCS1 |
| 218130_at | chromosome 17 open reading frame 62 | C17orf62 |
| 217736_s_at | eukaryotic translation initiation factor 2-alpha kinase 1 | EIF2AK1 |
| 209709_s_at | hyaluronan-mediated motility receptor (RHAMM) | HMMR |
| 204927_at | Ras association (RalGDS/AF-6) domain family (N- | RASSF7 |
| | terminal) member 7 | |
| 213945_s_at | Nucleoporin 210 kDa | NUP210 |
| 202178_at | protein kinase C, zeta | PRKCZ |
| 212886_at | coiled-coil domain containing 69 | CCDC69 |
| 215931_s_at | ADP-ribosylation factor guanine nucleotide- | ARFGEF2 |
| | exchange factor 2 (brefeldin A-inhibited) | |
| 205527_s_at | gem (nuclear organelle) associated protein 4 | GEMIN4 |
| 212431_at | KIAA0194 protein | KIAA0194 |
| 220564_at | chromosome 10 open reading frame 59 | C10orf59 |
| 207414_s_at | proprotein convertase subtilisin/kexin type 6 | PCSK6 |
| 201022_s_at | destrin (actin depolymerizing factor) | DSTN |
| 201613_s_at | adaptor-related protein complex 1, gamma 2 subunit | AP1G2 |
| 213947_s_at | nucleoporin 210 kDa | NUP210 |
| 206094_x_at | UDP glucuronosyltransferase 1 family, polypeptide | UGT1A1 /// |
| | A10 /// UDP glucuronosyltransferase 1 family, | UGT1A10 /// |
| | polypeptide A8 /// UDP glucuronosyltransferase 1 | UGT1A3 /// UGT1A4 |
| | family, polypeptide A7 /// UDP | /// UGT1A5 /// |
| | glucuronosyltransferase 1 family, polypeptide A6 /// | UGT1A6 /// UGT1A7 |
| | UDP glucuronosyltransferase 1 family, polypeptide | /// UGT1A8 /// |
| | A5 /// UDP glucuronosyltransferase 1 family, | UGT1A9 |
| | polypeptide A9 /// UDP glucuronosyltransferase 1 | |
| | family, polypeptide A4 /// UDP | |
| | glucuronosyltransferase 1 family, polypeptide A1 /// | |
| | UDP glucuronosyltransferase 1 family, polypeptide | |
| | A3 | |
| 218073_s_at | transmembrane protein 48 | TMEM48 |
| 202329_at | c-src tyrosine kinase | CSK |
| 206723_s_at | lysophosphatidic acid receptor 2 | LPAR2 |
| 40359_at | Ras association (RalGDS/AF-6) domain family (N- | RASSF7 |
| | terminal) member 7 | |
| 218115_at | ASF1 anti-silencing function 1 homolog B (S. cerevisiae) | ASF1B |
| 207416_s_at | nuclear factor of activated T-cells, cytoplasmic, | NFATC3 |
| | calcineurin-dependent 3 | |
| 204503_at | envoplakin | EVPL |
| 215125_s_at | UDP glucuronosyltransferase 1 family, polypeptide | UGT1A1 /// |
| | A10 /// UDP glucuronosyltransferase 1 family, | UGT1A10 /// |
| | polypeptide A8 /// UDP glucuronosyltransferase 1 | UGT1A3 /// UGT1A4 |
| | family, polypeptide A7 /// UDP | /// UGT1A5 /// |
| | glucuronosyltransferase 1 family, polypeptide A6 /// | UGT1A6 /// UGT1A7 |
| | UDP glucuronosyltransferase 1 family, polypeptide | /// UGT1A8 /// |
| | A5 /// UDP glucuronosyltransferase 1 family, | UGT1A9 |
| | polypeptide A9 /// UDP glucuronosyltransferase 1 | |
| | family, polypeptide A4 /// UDP | |
| | glucuronosyltransferase 1 family, polypeptide A1 /// | |
| | UDP glucuronosyltransferase 1 family, polypeptide | |
| | A3 | |
| 219935_at | ADAM metallopeptidase with thrombospondin type | ADAMTS5 |
| | 1 motif, 5 (aggrecanase-2) | |
| 219874_at | solute carrier family 12 (potassium/chloride | SLC12A8 |
| | transporters), member 8 | |
| 203573_s_at | Rab geranylgeranyltransferase, alpha subunit | RABGGTA |
| 213442_x_at | SAM pointed domain containing ets transcription | SPDEF |
| | factor | |
| 209425_at | alpha-methylacyl-CoA racemase /// C1q and tumor | AMACR /// C1QTNF3 |
| | necrosis factor related protein 3 | |
| 218295_s_at | nucleoporin 50 kDa | NUP50 |
| 204765_at | Rho guanine nucleotide exchange factor (GEF) 5 | ARHGEF5 |
| 203154_s_at | p21(CDKN1A)-activated kinase 4 | PAK4 |
| 213441_x_at | SAM pointed domain containing ets transcription | SPDEF |
| | factor | |
| 205309_at | sphingomyelin phosphodiesterase, acid-like 3B | SMPDL3B |
| 218931_at | RAB17, member RAS oncogene family | RAB17 |
| 203148_s_at | tripartite motif-containing 14 | TRIM14 |
| 214779_s_at | small G protein signaling modulator 3 | SGSM3 |
| 202364_at | MAX interactor 1 | MXI1 |
| 211952_at | importin 5 | IPO5 |
| 218518_at | chromosome 5 open reading frame 5 | C5orf5 |
| 205423_at | adaptor-related protein complex 1, beta 1 subunit | AP1B1 |
| 219188_s_at | MACRO domain containing 1 | MACROD1 |
| 211985_s_at | calmodulin 1 (phosphorylase kinase, delta) /// | CALM1 /// CALM2 /// |
| | calmodulin 2 (phosphorylase kinase, delta) /// | CALM3 |
| | calmodulin 3 (phosphorylase kinase, delta) | |
| 203215_s_at | myosin VI | MYO6 |
| 203214_x_at | cell division cycle 2, G1 to S and G2 to M | CDC2 |
| 50965_at | RAB26, member RAS oncogene family | RAB26 |
| 218387_s_at | 6-phosphogluconolactonase | PGLS |
| 212307_s_at | O-linked N-acetylglucosamine (GlcNAc) transferase | OGT |
| | (UDP-N-acetylglucosamine:polypeptide-N- | |
| | acetylglucosaminyl transferase) | |
| 212436_at | tripartite motif-containing 33 | TRIM33 |
| 218780_at | hook homolog 2 (Drosophila) | HOOK2 |
| 46142_at | lipase maturation factor 1 | LMF1 |
| 213622_at | collagen, type IX, alpha 2 | COL9A2 |
| 207901_at | interleukin 12B (natural killer cell stimulatory factor | IL12B |
| | 2, cytotoxic lymphocyte maturation factor 2, p40) | |
| 221592_at | TBC1 domain family, member 8 (with GRAM | TBC1D8 |
| | domain) | |
| 209379_s_at | KIAA1128 | KIAA1128 |
| 217551_at | similar to olfactory receptor, family 7, subfamily A, | LOC441453 |
| | member 17 | |
| 207165_at | hyaluronan-mediated motility receptor (RHAMM) | HMMR |
| 215249_at | ribosomal protein L35a | RPL35A |
| 205938_at | protein phosphatase 1E (PP2C domain containing) | PPM1E |
| 205231_s_at | epilepsy, progressive myoclonus type 2A, Lafora | EPM2A |
| | disease (laforin) | |
| 207833_s_at | holocarboxylase synthetase (biotin-(proprionyl- | HLCS |
| | Coenzyme A-carboxylase (ATP-hydrolysing)) ligase) | |
| 212070_at | G protein-coupled receptor 56 | GPR56 |
| 210181_s_at | calcium binding protein 1 | CABP1 |
| 214403_x_at | SAM pointed domain containing ets transcription | SPDEF |
| | factor | |
| 209367_at | syntaxin binding protein 2 | STXBP2 |
| 218779_x_at | EPS8-like 1 | EPS8L1 |
| 209624_s_at | methylcrotonoyl-Coenzyme A carboxylase 2 (beta) | MCCC2 |
| 212218_s_at | fatty acid synthase | FASN |
| 218248_at | family with sequence similarity 111, member A | FAM111A |
| 203431_s_at | Rho GTPase-activating protein | RICS |
| 208430_s_at | dystrobrevin, alpha | DTNA |
| 202721_s_at | glutamine-fructose-6-phosphate transaminase 1 | GFPT1 |
| 202605_at | glucuronidase, beta | GUSB |
| 200637_s_at | protein tyrosine phosphatase, receptor type, F | PTPRF |
| 210026_s_at | caspase recruitment domain family, member 10 | CARD10 |
| 200873_s_at | chaperonin containing TCP1, subunit 8 (theta) | CCT8 |
| 201021_s_at | destrin (actin depolymerizing factor) | DSTN |
| 91826_at | EPS8-like 1 | EPS8L1 |
| 216338_s_at | Yip1 domain family, member 3 | YIPF3 |
| 201189_s_at | inositol 1,4,5-triphosphate receptor, type 3 | ITPR3 |
| 219259_at | sema domain, immunoglobulin domain (Ig), | SEMA4A |
| | transmembrane domain (TM) and short cytoplasmic | |
| | domain, (semaphorin) 4A | |

TABLE 30
36 Genes generated by one of the two methods for identifying reactive stroma genes

| Probe.Set.ID | Gene.Title | Gene.Symbol |
|---|---|---|
| 204934_s_at | hepsin (transmembrane protease, serine 1) | HPN |
| 209426_s_at | alpha-methylacyl-CoA racemase /// C1q and tumor | AMACR /// |
| | necrosis factor related protein 3 | C1QTNF3 |
| 64486_at | coronin, actin binding protein, 1B | CORO1B |
| 203755_at | BUB1 budding uninhibited by benzimidazoles 1 | BUB1B |
| | homolog beta (yeast) | |
| 203317_at | pleckstrin and Sec7 domain containing 4 | PSD4 |
| 211576_s_at | solute carrier family 19 (folate transporter), member 1 | SLC19A1 |
| 202148_s_at | pyrroline-5-carboxylate reductase 1 | PYCR1 |
| 205339_at | SCL/TAL1 interrupting locus | STIL |
| 211984_at | calmodulin 1 (phosphorylase kinase, delta) /// | CALM1 /// CALM2 |
| | calmodulin 2 (phosphorylase kinase, delta) /// | /// CALM3 |
| | calmodulin 3 (phosphorylase kinase, delta) | |
| 217912_at | dihydrouridine synthase 1-like (S. cerevisiae) | DUS1L |
| 218275_at | solute carrier family 25 (mitochondrial carrier; | SLC25A10 |
| | dicarboxylate transporter), member 10 | |
| 202645_s_at | multiple endocrine neoplasia I | MEN1 |
| 209424_s_at | alpha-methylacyl-CoA racemase /// C1q and tumor | AMACR /// |
| | necrosis factor related protein 3 | C1QTNF3 |
| 206558_at | single-minded homolog 2 (Drosophila) | SIM2 |
| 219360_s_at | transient receptor potential cation channel, subfamily | TRPM4 |
| | M, member 4 | |
| 220584_at | hypothetical protein FLJ22184 | FLJ22184 |
| 201420_s_at | WD repeat domain 77 | WDR77 |
| 218683_at | polypyrimidine tract binding protein 2 | PTBP2 |
| 208190_s_at | lipolysis stimulated lipoprotein receptor | LSR |
| 219809_at | WD repeat domain 55 | WDR55 |
| 219395_at | RNA binding motif protein 35B | RBM35B |
| 207239_s_at | PCTAIRE protein kinase 1 | PCTK1 |
| 218180_s_at | EPS8-like 2 | EPS8L2 |
| 203287_at | ladinin 1 | LAD1 |
| 33814_at | p21(CDKN1A)-activated kinase 4 | PAK4 |
| 218365_s_at | aspartyl-tRNA synthetase 2, mitochondrial | DARS2 |
| 208824_x_at | PCTAIRE protein kinase 1 | PCTK1 |
| 219148_at | PDZ binding kinase | PBK |
| 201819_at | scavenger receptor class B, member 1 | SCARB1 |
| 218874_s_at | chromosome 6 open reading frame 134 | C6orf134 |
| 204532_x_at | UDP glucuronosyltransferase 1 family, polypeptide | UGT1A1 /// |
| | A10 /// UDP glucuronosyltransferase 1 family, | UGT1A10 /// |
| | polypeptide A8 /// UDP glucuronosyltransferase 1 | UGT1A4 /// |
| | family, polypeptide A6 /// UDP | UGT1A6 /// |
| | glucuronosyltransferase 1 family, polypeptide A9 /// | UGT1A8 /// |
| | UDP glucuronosyltransferase 1 family, polypeptide | UGT1A9 |
| | A4 /// UDP glucuronosyltransferase 1 family, | |
| | polypeptide A1 | |
| 217099_s_at | gem (nuclear organelle) associated protein 4 | GEMIN4 |
| 214393_at | Rho family GTPase 2 | RND2 |
| 204714_s_at | coagulation factor V (proaccelerin, labile factor) | F5 |
| 209972_s_at | JTV1 gene | JTV1 |

### Example 8

### Quantitative Tissue Imaging For Clinical Diagnosis and Prognosis of Prostate Cancer

### Specific Aims

Projects that use antibodies for clinical diagnosis or prognosis must take into account the huge biological differences that occur between patients and between clinical samples. One way to minimize the clinical variation is to use a panel of diagnostic or prognostic antibodies, each of which are known to capture relevant information in a subset of patients or a subset of clinical samples. However, there are also technical challenges that cause difference in staining within and between samples. One way to minimize the impact of technical variation would be to multiplex diagnostic and prognostic markers together with “reference” antibodies that that identify within tissues particular cell type rather than outcomes. These reference antibodies, under the same technical influences and in the same tissue section, can then be used to identify the signals observed for the diagnostic and prognostic antibodies of the relevant cell types which can then be quantified far more accurately than would be possible using separate hybridizations. In the case of prostate cancer, where diagnostic and prognostic antibodies are likely to be relevant in a highly variable and often rare fraction of the cancer cells or adjacent stroma cells in a patient or clinical sample, and where changes from normal tissue may often be subtle rather than “all-or-nothing”, it is likely that only the inclusion of reference antibodies in the same visualization will make it possible to identify the distinct clinically relevant regions with any confidence.

Fortunately, the technology that would be able to perform multiplex antibody staining of individual samples exists with the use of fluorescent dyes. The overall goal over this two phase project is to develop an automated quantitative image-based assay of the expression level of a panel of 5-10 diagnostic and 5-10 prognostic antibody biomarkers in Prostate cancer. Quantification of each antibody biomarker will be carried for specific cell types by utilizing co-localization of each test antibody biomarker of the panel with a reference antibody that is known to specifically identify total epithelium or tumor epithelial cells or tumor-adjacent stroma cells.

In Phase 1 of this project we will focus on the identification and characterization of the reference antibodies that reliably identify total epithelium or tumor epithelium or tumor adjacent stroma in both formalin-fixed and paraffin-embedded (FFPE) and frozen tissue sections. It is likely that a set of reference markers that distinguish different types of epithelial/tumor and fibroblast/smooth muscle stroma, could be useful for automated screening of samples for diagnosis. Phase II will then build on this reference set with additional markers of diagnostic and prognostic use.

In phase I, whole frozen and FFPE sections as well as prostate cancer tissue microarrays (TMAs) will be used to survey candidate reference antibodies and the reproducibility, variability, and accuracy of labeling will be determined for all cases of the TMA as well as by comparison to standard cell lines and normal prostate tissue specimens. This aim is non-trivial as antibodies can have optima for immunohistochemistry that differ markedly from each other. Optimizing a multiplex application may require examining may different types of antibody for each marker as well as a variety of conditions in order to uncover a standard conditions and a standard set of antibodies. Reproducibility, variability, and accuracy of the intensity data will be carefully assessed using positive and negative controls, TMA statistics, and repeated hybridizations on different days for adjacent slices of tissue, including the TMAs. Data storage consistent with the DICOM standard will take place by porting our data to a freeware database and visualization system (ConQuest).

The quantitative properties of the multiplex antibody system will be generated automatically using the proprietary scanning microcytometer developed by Vala Sciences Inc. using multiple fluorphores and validated by comparison to direct visual assessment of the binding location and intensity of representative candidate antibody biomarkers. Each section used for quantitative immunofluorescence (IF) will then be used to prepare DAB (bisdiazobenzidene) chromagen labeled version with hematoxyl counter stain and provided to a panel of four pathologists for estimation of labeling intensity and percent positively labeled epithelial cells or tumor epithelial cells or tumor-adjacent stroma cells. Visual scores for DAB and for fluorescence labeled sections will by quantitative compared to the automated output of the Vala system, using a linear model of the relationship between automated intensity and visual intensity. There is no strict necessity for an antibody to map exactly to a tissue type as assessed by a pathologist, but the scorings should be consistently different for any particular sample, in order to be confident that the antibody is measuring something slightly different, consistently. Zones of authentic tumor and stroma will be defined and the coincidence with colocalized pixels or cells will be quantitatively evaluated.

Workflow will be streamlined and then an SOP created to allow automatic image analysis to be completed with 4-5 days.

### B. BACKGROUND AND SIGNIFICANCE

### Overview

Despite advances in our understanding of cancer and the development of new therapeutics, cancer remains the number two killer in the US with mortality rates of many cancers remaining relatively unchanged for decades. Prostate cancer is the most common cancer and second leading cause of cancer-related death among males of Western countries [1-3]. While PSA screening has been a valuable marker increasing early detection of prostate cancer, PSA testing currently suffers from several limitations including lack of specificity and inability to accurately predict disease progression [1, 2, 4-8]. There is a critical unmet need to identify reliable novel biomarkers to assist in early detection of prostate cancer, and, most critically, to determine risk of prostate cancer rercurrence following initial therapy such as prostatectomy. Currently the major treatment modality for newly diagnosed prostate cancer remains radical prostatectomy. Radical prostatectomy provides an excellent outcome for organ-confined disease. However, 15%-20% or more of all surgical patients ultimately experience rercurrence indicating the presence of residual disease, local invasion and/or metastatic deposits at the time of surgery [7-11]. Traditional clinical parameters including tumor staging, Gleason score, and PSA levels, stage or their combinations based on preoperative values have not adequately predicted the patient risk of rercurrence [11, 12]. It is now recognized that prostate cancer exhibits hundreds of altered gene expression changes many of which may represent genes that directly influence outcome [13-19]. However a recent consensus statement by a panel of prostate SPORE leaders (the Inter-SPORE Prostate Biomarkers Study and NBN Pilot group) has tersely summarized that few or none have proven reliable enough to advance to clinical use (http://prostatenbnpilot.nci.nih.gov/aboutpilot ipbs.asp).

We are developing a new test using novel methods that identify cell-specific biomarkers that can be applied at the time of diagnosis to determine whether the tumor has the potential to recur after surgery. The development of a clinical test capable of distinguishing indolent and aggressive forms of the disease at the time of diagnosis will provide crucial guidance. First, this information will provide guidance as to who needs treatment thereby providing the option of avoiding surgery and the associated morbidity for those patients with a high risk of recurrence. Second, this information will also provide guidance as to who may profit from postsurgery or immediate adjuvant therapy thereby utilizing a period of many months or years during which recurrence otherwise could develop unopposed. Moreover, integration of gene expression signatures with clinical data has recently been shown to improve the accuracy of predicting progression, and metastasis [13, 14, 20]. One purpose of this proposal is the translation of a prostate cancer gene expression classifier into an antibody panel capable of rapid and reliable prediction of disease recurrence using (a) generally available clinical material such as biopsy specimens or, (b) as a guide to adjuvant therapy and patient counseling using post prostatectomy surgical pathology blocks. A crucial advantage of protein markers over RNA markers is that the protein markers provide spatial resolution of cell types and can detect cell-type-localized co-expression of markers, information that is lost in bulk RNA samples.

Moreover there remain critical challenges to diagnosis by biopsy. Over one million prostate biopsies are carried out per year in the U.S. Most are negative. Approximately 20% of these negative biopsies are judged insufficient for a definitive diagnosis owing to small foci or read as “atypical glands” only seen or other ambiguities, i.e. ˜100,000 such cases per year. The microenvironment of these sites contains potential information for diagnosis. We have observed that the tumor adjacent stroma of prostate cancer exhibits hundreds of altered mRNA expression changes and have derived a gene list that accurately identifies tumor adjacent stroma tissue. Thus, antibodies of selected gene products may be potentially useful to assist in diagnosis of traditionally nondiagnositic biopsies.

### Importance of Identifying Diagnostic and Prognostic Prostate Biomarkers.

To date, only a limited number of diagnostic biomarkers that are differentially regulated in prostate carcinoma have been identified such as prostate-specific antigen [2, 5, 6, 23-25], prostate specific membrane antigen [26, 27], and human glandular kallikrein 2 [10, 28-32], and PCA3. While these antigens have been useful in the development of early diagnostics and for the directed delivery of therapeutics to prostate cancer in preclinical models [33, 34] these markers do not address the need to identify biomarkers that characterize early or advanced stages of prostate carcinogenesis and metastasis. Recent studies have identified circulating urokinase-like plasminogen activator receptor forms that may be used alone or in combination with other prostate cancer biomarkers (hK2,PSA) to predict the presence of prostate cancer [35]. Other potential prognostic markers include early prostate cancer antigen (EPCA), AMACR, human kallikrein 11, macrophage inhibitory cytokine 1 (MIC-1), PCA3, and prostate cancer specific autoantibodies [5, 36-42].

The search for novel prostate cancer biomarkers has turned to the use of global genomic and proteomic profiling to facilitate the discovery of multiple markers with both diagnostic and prognostic significance [5, 18, 36-42]. Gene-expression profiling comparing gene expression from normal prostate tissue, BPH tissue, and prostate cancer tissue has identified many potential genes that are differentially regulated in prostate cancer [14, 15]. These include hepsin, a serine protease, alpha-methylacyl-CoA racemase (AMACR), macrophage inhibitory cytokine (MIC-1), and insulin-like growth factor binding protein 3 (IGFBP3) [40], TGFβ1, IL-6, and many others. Validation of these markers at the protein level from patient tissue or serum samples and clinical validation of these markers as true diagnostic and prognostic tools are necessary. While some of these candidates have appeared in meta analyses (e.g., Rhodes, 2002), as noted, the recent consensus statement of the InterSPORE study has noted that none have proven sufficiently reliable for clinical use and none have been used to form a panel that predicts outcome of multiple independent case sets.

Current clinical parameters including Gleason score, PSA, and tumor staging have been inadequate in predicting patient outcome. Combinations of clinical criteria have been assembled into predictive nomograms in attempts to improve diagnosis of indolent vs. advanced disease [11, 12]. While these studies suggest improved diagnostic and prognostic capabilities, those based solely on preoperative clinical values perform less well and they await widespread clinical validation. One major challenge has been that the majority of prostate cancers share similar histological features (Gleason score) or clinical markers (PSA) but exhibit widely different clinical outcomes. Recently multigene profiles of biomarkers that are predictive of the outcome of prostate cancer at the time of diagnosis have been developed [14, 20, 44-46]. Singh identified a 5-gene classifier capable of predicting prostate cancer recurrence better than clinical parameters of preop PSA or tumor stage [46]. Stephenson identified a set of 10 genes highly correlative with prostate cancer recurrence. An analysis combining clinical variables with the 10-gene classifier greatly improved prediction of clinical outcome [20]. Henshall identified >200 genes that correlate with prostate cancer recurrence better than preoperative PSA [14]. From these studies it is clear that molecular correlates have the potential to provide a considerable increase in information related to outcome than current clinical parameters. In addition to prediction of outcome, it is likely that several of these unique biomarkers are functional and therefore provide intervention opportunities. The proper identification of the molecular determinants predictive of prostate cancer rercurrence, their validation at the protein level, and the translation of the data into a robust clinical test is the challenge addressed in our current proposal. We have developed improvements in both the identification and validation of candidate genes that will enable a rapid and robust transition to a clinical test.

### Improved Gene Lists

We have developed new methods that have helped in the development of gene signatures for the diagnosis and for prognosis based on expression values of tissue obtained at about the time of the original diagnosis. First, as described herein, we have used a linear combination model together with knowledge of cell composition as determined by a panel of four pathologist to determine gene expression by cell type [18]. These studies revealed cohorts of genes that are differentially expressed by tumor epithelium compared to epithelium of PBH or dilated cystic glands or stroma [18]. This observation has important practical considerations. While most global genome studies have looked at differences between normal and cancerous prostate epithelial cells, considering the contribution of stromal cells as “contamination”, we have found that stroma exhibit dozens of significantly differential gene expression changes between tumor-adjacent stroma and stroma remote from tumor sites [18] and dozens of differential expression changes between tumor-adjacent stroma of recurrent PCa cases compared to nonrecurrent cases [43]; [44]. We have identified two separate subsets of genes. The first consists of tumor epithelium specific and stroma cells specific genes that are differentially expressed between recurrent PCa (“aggressive” cancer, relapsed PCa) and nonrecurrent PCa (“indolent” cancer, nonrelapsed PCa). Since nearly all PCa tissue specimens contain stroma or reactive stroma in the immediate microenvironment of tumor, the proper inclusion of antibodies sensitive to stromal change provides an important ingredient of a “classifier” for prognostic use. These expression changes may be used to predict outcome ([43] [44]).

Second, we have identified a separate subset of tumor-adjacent stroma specific genes. These genes are differentially expressed between tumor-adjacent stroma and remote stroma. These expression changes may be used to detect tumor-adjacent stroma at foci of “nondiagnostic” or “atypical” tumor in biopsies of equivocal cases thereby potentially converting “nondiagnostic” cases to a definitive determination. We propose to use these gene lists as the starting point for the development of panels of 5-10 antibodies for application to biopsy or postoperative FFPE tissue specimens that are routinely available for all patients with a confirmed or suspected diagnosis of prostate cancer. While RNA may be retrieved from these samples, the preservation of a particular set of transcripts with the crucial information in all cases and in proportion to the amounts in fresh tissue is problematic. In contrast, antibody based diagnosis from FFPE is well established. In Phase II we plan to utilize a high throughput scanning microscope to identify the best antibodies for inclusion in the panels. TMAs consisting of 254 prostate cancer cases, normal prostate tissue and defined cell lines will be used for the survey. The TMAs to be used here have been constructed to contain cores especially rich in tumor-adjacent stroma and remote stroma. These cores will allow us to evaluate whether the differential expression observed between relapsed and nonrelpased cases may be observed in adjacent nontumor tissue or even in remote nontumor tissue and to confirm that diagnosis based on tumor-adjacent stroma is reliable.

Additional potential applications include the detection of tumor-adjacent stroma in “negative” biopsies that may have narrowly “missed” frank tumor. This possibility is of considerable significance given that most of the million biopsies performed each year are “negative”.

### Biomarker Validation Using Tissue Microarrays (TMAs).

The heterogeneous nature of DNA changes in prostate cancer makes it unlikely that a single biomarker will be adequate for proper determination of prostate cancer severity and risk of rercurrence. What is needed is the identification of a panel of biomarkers that can be shown to correlate with different aspects of disease progression and risk of rercurrence in the population of cancer patients. The screening of tissue by use of microarrays (TMAs) is ideal for identification of markers that statistically correlate with disease progression and outcome [45-48]. Screening of TMAs is a powerful tool for validation of the microarray results, for extension of the RNA expression results to protein expression and for the identification of antibodies of biomarkers that are widely expressed and readily available from samples routinely taken at time of diagnosis. TMAs are constructed using hundreds of different patient samples that span the entire range of clinical pathology and outcome. Furthermore, it requires only small amounts of tissue that can be collected at the time of diagnosis such as biopsy samples and is amendable to high throughput analysis using multiple antibody probes. TMAs may be made from selected archived cases with clinical annotation spanning many years detailing survival and other parameters, such as treatment history.

Numerous studies have used TMAs to identify or validate prostate cancer biomarkers associated with disease progression, response to therapy, rercurrence, and metastasis [45-48, 49, 50]. TMA analysis was used to validate a seven antibody panel derived from a 48 gene expression signature enabling more accurate classification between Gleason grade 3 and 4 tumors [47]. Multiple TMA studies have identified several markers indicative of prostate cancer progression including Amacr (alpha-methyl acyl racemase) AMACR, AR, Bcl-2, CD10, ECAD, Ki67, and p53 [45]. TMA analysis has identified 13 genes associated with prostate cancer rercurrence. These include AKT, □-catenin, NFκB, Stat-3, hMSH2, Hepsin, PIM1, syndecan-1, Bcl-2, Ki67, and ECAD [45]. Few have been formed into a coherent predictive panel and evaluated as a panel. Therefore, the performance of a panel compared to individual antibodies and the potential of combinations to overcome the diversity of prostate cancer is unknown. Nearly all studies ignore the stroma although smooth muscle alpha actin has been examined by Rowley and coworkers [51]. Others suffer the caveats noted by interSPORE group. Several, such as AMACR are utilized as an aid to diagnosis in surgical pathology but are not used routinely in risk assessment. We propose the systematic evaluation of over 50 predicted prognostic biomarkers (Phase I and Phase II) taken from a predictive panel of known performance at the RNA level.

### High Throughput Analysis and Quantification.

The current study will address several obstacles that have precluded the development of a rapid and reliable biomarker panel ready for clinical testing. While TMAs contain a wealth of potential data, the ability to properly identify and quantify the cell-specific staining patterns of antibodies currently relies on manual identification or pattern recognition programs that are both time consuming and subject to bias and error. Therefore we will utilize an automated digitizing scanning system developed by Vala Sciences Inc. (http://www.valasciences.com/). This system can rapidly record histological sections labeled with up to 10 distinct fluorophores with pixel level subcellular resolution including for TMAs and display each color separately. The system has been acquired by Beckman Coulter Instruments Inc. (Fullerton, Calif.) (http://www.beckmancoulter.com/hr/pressroom/oc pressReleases detail.asp? Key=4764&Date1=12/11/2003) and developed as the Beckman-Coulter IC 100 system. Our application requires only two colors. The reference antibody will be applied to locate all epithelial cells or the subset of epithelial tumor cells or stroma cells and a test antibody will be applied in with a second fluorophore and the pixels of colocalization of test antibody with bona fide epithelia or tumor or stroma will be determined as well as the pixels of not colocalized with target cells. The intensity of antibody labeling at target sites will then be integrated, normalized and compared to nonlocalized binding or to the known clinical outcome. Thus specificity, sensitivity, and accuracy may be determined by existing technology and software. As a gold standard, Phase I will establish the utility of the reference antibodies in comparison to the visual results of a panel of pathologists.

### Phase II Studies

Development of clinical studies. Phase II will involve forming and validating the multiplex application of antibodies as prognositic panel and as a diagnositic panel in clinical trials. The diagnoistic and clinicaol performance of candidate antibodies will be determined. Teo pandel will be formed composed of antibodies with (1) maximum performance by the criteria of intensity, specificity, and sensitivity and (2) superior accuracy with subsets of cases not equally achieved by other antibodies.
Acquisition and tests of monoclonal versions of panel members. All polyconal antibodies will be converted to monoclonal counterparts by commercial license from existin vendors or commission using sources that can provide GMP product. GMP manufacture of the predictive antibody will be initiated and a clinical protocol developed for recruitment and testing on prostate cancer patients in a CLIA setting.
Expansion of biomarker discovery/validation platform; In Phase II we will continue to validate novel prostate cancer gene classifiers on an expanding set of TMAs. We will also examine whether circulating protein biomarkers have predictive value.

### C. PRELIMINARY DATA

### C.1. Derivation of Diagnositic and Predictive Genes Signatures.

While the importance of the tumor microenvironment on tumor progression and metastasis has been well documented [19, 40, 49, 51-54], very few studies such as Tuxhorn et al. (2002) [51] and [55] have identified genetic markers of reactive stroma. We have utilized linear regression to define expression profiles of the four major cell types contained within prostate tissue samples including tumor cells, stromal cells, and two additional normal epithelial components [18]. In the linear model, the observed expression of any gene (the expression array result for that gene) in a complex piece of dissected prostate tissue used for RNA preparation and Affymetrix analysis is considered to be due to the sum of contributions from the principal cell types in the sample. Each contribution is in turn due to the proportion or percent of each cell type in the sample and the characteristic expression coefficient for the particular gene in a particular cell type:

Gᵢ=β′_{tumor,i}Pₜᵤₘₒᵣ+β′_{stroma,i}Pₛₜᵣₒₘₐ+β′_{BPH,i}P_{BPH}+β′_{dilcys gland,i}P_{dilcys gland}. (egn. 1)

where Gᵢ is the observed Affymetrix total Gene expression, β′ are the cell-type specific expression coefficients, and the P's are the percent of each cell type of the sample used for the array. The percentages, P, may be determined by examination of H and E slides of the tissue used for RNA preparation by a team of four experienced pathologists. The expression coefficients are determined by multiple linear regression (MLR) analysis. For grossly microdissected tissue enriched in tumor, there are four major cell types as expressed in eqn. 1. We showed that there is very high and statistically significant agreement both between and amongst the four pathologists for the determination of cell-type percentages [18]. In this initial study we sought to determine genes that were consistently expressed predominately by one cell type or another without regard to outcome, i.e. genes that were characteristic of cell type in prostate cancer specimens. We observed 3384 genes were statistically significantly expressed predominately by one cell type. For example, 1096 were consistently expressed by tumor epithelial cells while 496 genes were significantly associated with BPH epithelial cells. Cell type specific expression has been validated by comparison to the literature, by quantitative PCR of LCM samples, and by immunohistochemistry [18].

C.1.A. Diagnostic multigene signature. These initial studies indicate that numerous, perhaps hundreds, of genes may be differentially expressed in the microenviroment of tumor cells which may be useful in diagnosis in supplement to or even in the absence of data from the tumor cell component [18]. Three methods have employed to identify such genes. We adopted the model that it is mainly tumor-adjacent stroma that exhibits the most and largest differential expression changes between the microenviroment around tumor cells and normal or remote stroma. We also assumed that stroma remote from tumor sites of PCa-bearing prostate glands could be used to approximate the expression of normal stroma. We utilized publicly available expression data from 91 cases applied to 148 U133A Affymetrix GeneChips (GEO accession number GSE8218). These cases were the same as those previously studied on the U95av platform [18] plus additional cases. The percent cell composition determined exactly as described [18]. The goal is to find the genes that have altered expression levels between normal stroma cells and the stroma cells close to the tumor cells. We divided U133A samples into two subgroups: 91 tumor-bearing cases and 57 non-tumor-bearing portions of tissue from the same cases. These portions are largely remote stroma. We then applied eqn. 1 to each set thereby determining two β values for stroma: tumor-adjacent stroma and tumor-remote stroma. Note that neither recurrence status or any other clinical parameter such as the Gleason score indicating differences among the tumor bearing portions was considered. Thus only β characteristic of stroma were determined together with a least-squares estimate of error for each β value. Note also that β which are large relative to error must be uniformly or characteristic of tumor-adjacent stroma or remote stroma, i.e. independent of clinical values such as Gleason scores that might indicate differences in aggressiveness. Such β favor high T values in significance tests. The significant differences between the β values for tumor-adjacent stroma and remote stroma were determined. This method produced 208 genes. These significant genes are candidate genes as specifically differentially expressed in the tumor-adjacent microenvironment.

In a second method eqn 1 was extended to include a cross-product:

$${G}_{i} = {β}_{tumor , i}^{′} {P}_{tumor} + {β}_{stroma , i}^{′} {P}_{stroma} + {β}_{BPH , i}^{′} {P}_{BPH} + {β}_{dilcys gland , i}^{′} {P}_{dilcys gland} , + {β}_{stroma , i} ( {P}_{stroma} * {P}_{tumor} ) , \qquad Eqn 2$$

The cross-product term is used for modeling the interaction between tumor and stroma cells. The significant interaction can be treated as the altered expression trait of stroma caused by the adjacent tumor cells. Egn 2 was applied to the U133A plus data set thereby 1820 significant cross-product terms (˜8% of the probe sets). Finally a third gene list was determined by application of Egn. 2 to and independent set of 91 cases measured on the pangenomic Affymetrix U133A plus2 GeneChips (unpublished data, D. Mercola). This third data set could be used as a test set for the genes determined using the U133A arrays however the differences in platform means that testing can not be applied without cross platform normalization, a process that introduces additional error. Therefore we applied eqn. 2 to the third data set ab initio and sought genes that met the same significance criterion yielding 4533 significant cross-product terms (also ˜8% of probe sets).

Finally we asked which of these genes were common with to all three determinations (the maximum intersect is 208 genes). This three-way intersect yielded 90 genes, i.e. 90 genes which appeared on all three calculations using the two different case sets. These genes may be used to diagnosis the presence of tumor-adjacent gene changes entirely from stroma tissue in the absence of tumor cells.

To test the consistency of these genes PAM (Prediction Analysis for Microarrays) was employed using all 90 genes as a classifier to distinguish tumor and nontumor tissues of the U133A and the U133 plus2 data sets. This method does not utilize information of percent cell type composition.

First, we extracted relevant expression values for these 90 genes from U133plus2 data as a training set. Then we used PAM to analyze these extracted expression data, with tumor/non-tumor as relevant classification variable. Via cross validation, PAM identified 21 genes out of 90 as the best predictor for classification variable. The classifier was tested on the U133A data which yielded a specificity of 100% and a sensitivity of 94.4% (accuracy>94.4%).

Conclusions. The observations indicate that it is possible to diagnosis the presence of prostate cancer in a large proportion of cases solely from an analysis of the expression of tumor-adjacent tissue, i.e. in the absence of tumor cells. This has a very important potential application to the understanding of patient biopsy material. Moreover, by repeating the above analysis by applying egns. 1 and 2 only to U133A, (two list input in forming the intersect) the final analysis would be free of any input from the test set and stringently objective. We plan to the 21 gene set in this way and to use the resulting list as the starting point for the identification of antibodies suitable for formation of a diagnosis panel for Phase II.

C.1.B. Prognostic multigene signature. MLR may be extended to identify genes differentially expressed by a given cell type between indolent and aggressive tumor cases where “aggression” is defined by chemical recurrence. In the simplest application of this method, eqn. 1 is applied separately to each class of cases—indolent or aggressive cases—and significant differences in β for these two classes of cases for each cell type are determined. Using these methods for a series of 91 patients examined on 131 U133A GeneChips, we observed 1212 genes were significantly and differentially expressed by tumor cells (p<0.05).

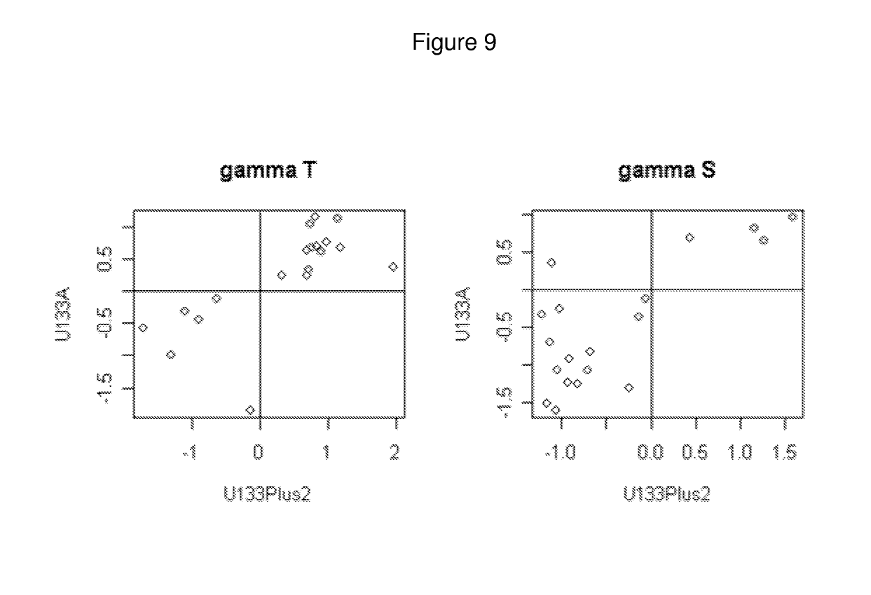

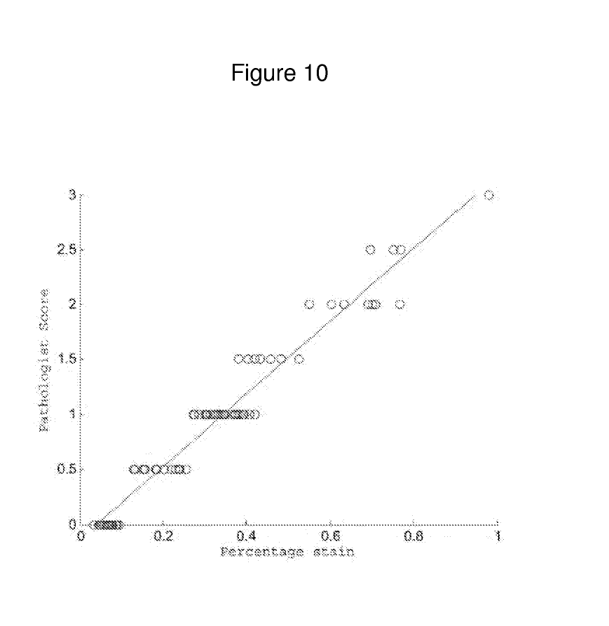

In order to validate these differential expression changes, the process was then repeated using the independent 86 cases assessed on the U133A plus2 platform. Again, no cross platform normalization is required. 1373 significantly differentially expressed (p<0.05) genes were identified. “Validated” genes were then defined by four criteria: (i) two or more probe sets of each platform mapped to the same gene; (ii) where multiple probe sets for the same gene were present, all probe sets for the same gene met criteria (iii) and (iv); (iii) differential expression changes for each case set were significant with p<0.05, (iv) the differential expression of identified genes are in the same direction for each case set. We observed that 18 tumor cell specific genes and 19 stroma cell specific gene met these criteria. The chances that that 37 genes could appear to meet the significance criteria for both case sets and be of the same sign by chance is a vanishingly small p<zx indicating supporting that the validated gene list is specific. Moreover, the magnitude of differential express of these genes for the two cases sets is positively and significantly correlated (FIG. 9) further demonstrating the relatedness of the validated genes. None of the genes are the same as those determined for the diagnostic multigene signature.

Conclusions. These preliminary calculations indicate that it is readily possible to identify multigene signatures that exhibit reproducible differential expression changes that discriminate indolent for aggressive disease. These calculations account for the cell type heterogeneity that is an essential part of the structure of prostate cancer and leads to the heterogeneity of sample collections assessed by others. Therefore our approach may overcome a major problem plaguing the development of a reliable prognostic classifier. In addition we employed two independent data sets. As a result of accounting for percent cell type composition, we have observed separate gene signatures for tumor epithelial cells and for tumor-adjacent stroma cells. Thus, it may be possible to utilize tissue with sparse tumor content to enhance the prognostic value of the specimens. We plan to use the 38 identified genes as a starting point for the identification and screen of antibodies for our antibody panel in Phase II. This study with TMAs will further validate the prognostic properties of our signature. Numerous additional studies are in progress. We need to test our classifier on published independent data sets by calculation of operating characteristics. We plan to use PAM to further refine our gene list and assess the accuracy by as for the diagnostic profile. These and other refinements are in progress.

C.2. Fully Automated Fluorescence and Absorption Microscopy Analyses. The scanning microscopy and separate image representation from multiple color labeled slides to be used here has been developed by Vala Sciences Inc. of San Diego by J. Price, President and CEO, and co-workers and has been utilized for a variety of publications (61-84). This system, known as the Q3DM Eidag™ 100 robotic microscopy instrument runs on the Beckman Coulter's CytoShop™ version 2.0. This instrument includes a Nikon (Melville, N.Y.) Eclipse microscope with an automated stage interfaced to a fluorescence light source and filter wheel of up to 10 narrow band base optical filters in the range 413 nm-663 nm. Numerous supporting software packages has been developed. The system is supported by a variety of antibody-based kits prepared by Vala. Each product contains staining reagents that are targeted towards particular proteins of interest along with a software program (Thora™) that can be used on virtually any computer system. The original instrumentation was developed by a predecessor company, Q3DM Inc. by J. Price focused on the development of high throughput microscopy instrumentation oriented primarily toward automated fluorescence image cytometry (61-84). This instrumentation was designed with accurate image segmentation (81, 83, 84), fluorescent excitation arc lamp stabilization (68, 82), and autofocus for producing fluorescence imaging (69). This system was sold to Beckman Coulter and developed as the Beckman-Coulter IC 100. The current instrumentation is a further generation scanning microcytomer and includes a slide holder hotel for automated scanning of 100 prepared slides.

Two modes, immunofluorescence (IF) with fluorophore-labeled antibodies and immunohistochemistry using absorption chromophores will be employed in the present study. For both methods spectral separation of multiple labeled sections is achieved by capturing multiple images using multiple fixed band pass filters. Up to ten fixed band pass filters are automatically rotated into the optical path of the light either in front of the light source or in front of the camera. Therefore up to 10 images per section are recorded on a monochrome CCD camera creating a “spectral stack”. Spectral unmixing from the data of the spectral stack is sensitive to errors in registration of images of the spectral stack to chromatic aberration. Multiple precautions have been included in the software correct for effects.

For IF the narrow emission of fluorophores of different colors are resolved directly by the appropriate filter of the spectral stack and the corresponding image may be used for pixel-level analysis (for examples see Progozhina et al 2007).

For IHC the broad absorption bands of typical chromophores such as DAB (bisdiazobenzidene), hematoxyln, and others require analysis of multiple images of the spectral stack as previously developed (3). Briefly, spectral unmixing of the observed intensity is based on a model expressed in matrix notation as a linear combination of chormophores where each chromophore contribution is the product of amount of binding and fluorescence intensity or absorption in a given wavelength range. Emission and absorption spectra for all chromaphores to be used here are known and the desired unknown are relative amounts of each chromaphore contributing to a given pixel intensity. These are determined by the method of Non-negative Matrix Factorization (NMF) (Rabinovitch et al. unpublished). Effective multicolor separation of tissue images usually requires knowledge of the individual chromaphores interacting with the tissue. Based on NMF, the Vala system is the first system capable of performing this color decomposition in a fully automated manner without reference to individual chromaphore-tissue absorption or fluorescence spectra. Instrumentation and software implementing these methods have been developed, characterized and validated on TMAs using objective standards and expert visual scoring and the results are described in reference (Rabinovitch et al. unublished, Rabinovich et al. 2006).

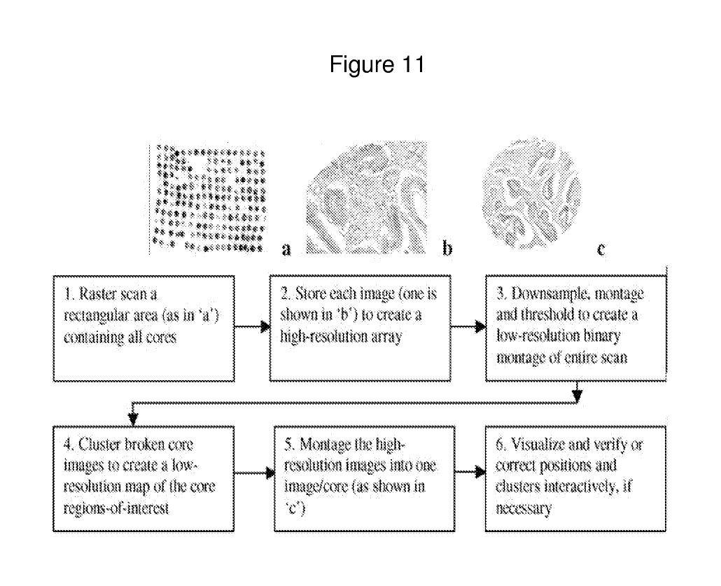

Supportive additional features of imaging technology and software include: (i) the ability to regroup broken core images which are common in TMA fabrication. None of the currently available software other than that of Vala has addressed this to our knowledge. This problem solved this problem by using the K-means clustering algorithm (53, 54), which provides an automatic method for grouping objects (e.g., pixels) based on distance. Details can be found in the Vala TMA software “framework” article (Rabinovich et al. 2006). (ii) Online viewing, computerized entry of TMA Scoring and Storage is implemented. The tissue microarray core images are organized by software for viewing, interactive entry of expression scores and storing of the data in an organized format. The user can click on any of these thumbnails to view an enlarged image of the entire core and/or a full magnification subfield of the image of the core. Data can then be entered by selecting the data entry pop-up window. The storage format for the images is standard TIF or BMP. Further details can be found in reference (Rabinovich et al. 2006). (iii) Fully Automated Densitometry IF- or IHC-labeled TMAs using Unsupervised Multispectral Unmixing has been developed and implemented (Rabinovich et al. 2006). FIG. 11 summarizes major steps in data acquisition and analysis.

We propose to utilize reference antibodies in one color to identify particular cell types and double label the same section in a second color to localize a candidate or test antibody binding. The amount of test antibody binding to target cells such as tumor cells will be determined by colocalization: determination of the pixels of test antibody binding at the site (pixels) of reference antibody labeling. The integrated pixel values of non-colocalized test antibody also will be determined as a measure of lack of specificity.

Two separate uses of colocalization are planned. For routine high throughput screening of candidate antibodies (Phase II), IF will be used as IF has is more sensitive, enjoys greater dynamic range and more amenable to the application of multiple proven antibodies to patient material. For characterization of reference antibodies (Phase I) by comparison to the gold standard of visual score by an expert panel of pathologist, IHC will used in order to provide slides that can be directly assessed by pathologists and compared to the results of colocalization by spectral deconvolution.

C.3. Accuracy of Spectral Unmixing of IHC labeled TMAs: comparison to single labeling and to visual scoring. Cell type specific labeling of candidate biomarkers in an automated fashion proposed here relies on colocalization of candidate antibodies with the cell of interest as identified by a reference antibody using a second color. The resolution of separate fluorphore labeling patterns from multiple labeled tissue section may be obtained directly from images of multiple narrow band base filters. However absorption/transmission based images of IHC are more challenging and require spectral separation using nonmatrix factorization (NMF). We have evaluated this approach by using double labeled TMAs by the following procedure. Using a set of 97 cores, we first applied the DAB stain and captured 437 multispectral image stacks 9), an average of 4.5 fields of view per core. We then added the hematoxylin stain and acquired a second image stack. The second stack served as the input to our algorithm and the resulting decomposition, which estimated the DAB staining, was compared with the first stack, which serves as the ground truth. We then experimentally evaluated the use of NMF for the color decomposition problem. While reconstruction error represents a quantitative measure, it does not provide a standard for judging how accurately the estimated components represent the dye concentrations. We quantified the performance by comparing the ground truth single-stained image to the corresponding automatically extracted component of the doubly-stained tissue sample as proposed by Rabinovich et al. (Rabinovitch et al. unpublished).

Using this procedure the average decomposition error over all samples was 6.73% with standard deviation of 1.81%. This therefore provides one objective assessment of the accuracy of spectral devolution in comparison to the single chromophore labeled section.

With the accuracy of densitometry via multispectral unmixing established, we asked how this quantitative measurement compares with the subjective scoring of a human expert. A panel of four trained pathologists (M. Krajewska, S. Krajewski, D. Mercola, A. Shabaik) evaluated the 97 tissue biopsies for the expression of antibody protein (DAB). The scoring was performed according to pathology conventions and each tissue section was graded on a scale from 0.0 to 3.0 in increments of 0.5. For correlation of the visual and analytical results, we analyzed the performance of a linear model y=mx+c, where x is the score reported by NMF decomposition, y is the pathologist's score, m is the slope and c is the y-intercept. Linear regression was used to fit the model. The fitting error for regression may be an indication of the prediction error of the model. However, depending on the complexity of the model and the amount of data available, the regression error can be significantly different from the true prediction error of the model. Thus, an effort was made to estimate the prediction error and report it instead of the fitting error. The simplest and most widely used method for reporting prediction error when the data is scarce is cross-validation (86). Ten-fold cross validation resulted in a mean squared error of 0.02 with a standard deviation of 0.01. This is equivalent to a root mean squared (RMS) error of 0.163, which also translates to an average of 5.4% error on the pathologist's scale. A major result of the validation study is that the 5.4% error is considerably larger than the corresponding signal:noise ratio of the camera detector. Thus the validation makes available a greatly increased dynamic range of electronic signal detection of the camera-based microscope over the visual system with a “noise” value of ˜3×5.4%=16.2% vs.<1% for the camera. The increased dynamic range for quantified antibody binding overcome a major limitation of antibody labeling using visual or IHC methods and greatly increases the ability to identify antibodies that correlate with survival data and other important clinical co variants. This advantage is extended many times for fluorescence-based antibody labeling.

Another decomposition of the form A=BC that is widely used is Independent Component Analysis (ICA) (Hyvarinen, J., Karhunen, and E. Oj a, Independent Component. Analysis, John Wiley & Sons, 2001). ICA is based on the assumption that the matrix A is the result of the superposition of a number of stochastically independent processes. This is a more reasonable description of the staining process where each stain can be assumed to be independent of the other stain. Classically, however, ICA algorithms do not enforce non-negativity and that makes them unsuited for stain recovery as well. We experimentally evaluated the use of NMF and ICA for the color decomposition problem. While reconstruction error represents a simple quantitative measure, it does not provide a standard for judging how accurately the estimated components represent the dye concentrations. We quantify the performance by comparing the ground truth single-stained DAB image to the corresponding automatically extracted component of the doubly-stained DAB/hematoxyln tissue sample. Quantitatively, the overall for four images sets was 50% larger for ICA compared to NMF (the images are available at hppt//vision.ucsd.edu/). Both NMF and ICA provide good results however there is an observable increase in fidelity to ground truth for the NMF analysis. We propose to utilize NMF for the studies proposed here.

Conclusions. 1. These studies provide support for the ability to successfully decompose multicolor labeled TMAs to component images. The application proposed here is simpler as separate 2D images are unnecessary. We plan to extract a subset of pixel intensities, those of chromaphore A that are co-localized with the pixels of chromaphore B where chromaphore A predominately binds to cells of interest such as tumor or epithelial cells or stroma cells. We have not completed this task however only minor modifications to existing software, pixel integration, is required and is proposed as a milestone of Phase I. The data of co-localized chromaphore B, the test chromaphore, would then be analyzed by Cox-regression and ANOVA analysis with covariates of disease progression currently available for the cases of the PCa TMA. 2, The automated ability to scan TMAs and extract quantified data will greatly facilitate antibody screening.

C. 4. Multicolor IF separation at the subcellualar level. The design goal of the Vala scanning robotic microscope is subcellular segmentation using pixel level resolution. It is important to note, therefore, that this capability exceeds the needs of cellular resolution required here which is well within current level of the instrumentation development. This was insured by the successful development of an automated membrane algorithm of the Thora package (Prigozhina 2007). For example mouse skin tumors were labeled with three fluorophores, two to identify proteins of interest, the membrane binding E-cadherin and the epithelial localizing antibody anti-K-14, and a cell localizing label for nuclei, DAPI. In this context, K14 is a putative marker for tumorigenic epidermal cells that invade the deeper skin layers. Cells exhibiting K14 signal (high red channel fluorescence) were clustered within the tumor loci. Areas of the section that stained brightly for K14 stained relatively dimly for cadherins, whereas surrounding tissue stained poorly for K14 and brightly for cadherins. To quantify K14 and cadherins, Thora separated the three primary cellular compartments (membrane, nucleus, and cytosol) from the dualcolor image of pan-cadherin and nuclear fluorescence. Thora estimated the cell boundaries in both the normal cells bordering the tumor where the cadherin signal was strong and in the tumor where it was relatively weak. To measure cadherin reduction in K14-positive cells, TMIs (total membrane intensity by pixel integration by boundary recognition) in the cadherin channel were collated for K14 cells with ACI (average cytoplasmic intensity) of 30 (the ACI range was 0 ACI-255 for the 8-bit images). By visual inspection and comparison of the intensity measurements of different cellular regions, ACI values below 30 arose from background staining that was not cell-specific. The mean pan-cadherin TMI for K14-positive cells was just 34% of that for K14-negative cells, and this difference was highly significant (P<0.01). Thus, the K14-positive cells representing invading tumor exhibited quantifiably reduced cadherin expression relative to the surrounding cells. Other examples and details of the development have been described in detail (Prigozina 2007).

For the applications proposed in this SBIR project membrane boundary recognition is less crucial as it is only necessary to identify zones of tumor epithelial cells and zones of nonepithelial stroma and those subareas of test antibody labeling that colocalize with either tumor or, for nonspecific labeling nontumor labeling. It is of course important to recognize that colocalized tumor labeling may only be increased on average compared to non tumor labeling and, like cadherin, this may be readily quantified.

### C. 5. TMA Construction.

The Prostate cancer TMAs to be used here have been fabricated as part of the NIH-supported UCI SPECS (Strategic Partners for the Evaluation of Cancer Signatures) consortium at the Burnham Institute of Medical Research, a consortium member of the UCI SPECS program and are available here as an NIH resource of NIH-sponsored projects. The TMAs have been specifically fabricated to validate the cell-specificity of candidate biomarkers of prostate cancer. 272 cases with known clinical outcome have been included to date. FFPE blocks and clinical follow-up were retrieved from two participating institutes of the SPECS consortium according to an IRB-approved and HIPPA-compliant protocol and consist of cases provided by SKCC (60 cancer cases, 12 normal cases) with the rest of the cases drawn from UCI that have 10-19 years of clinical follow-up with clinical characteristics as previously described in T. Ahlering and coworkers [75]. All cases have been re-examined by two clinical pathologists who confirmed the Gleason score and defined areas of tumor, BPH, stroma adjacent to tumor, stroma away from tumor, and epithelium of dilated cystic glands and PIN cores. In order to validate cell-specific binding properties of candidate biomarker antibodies, each case on the TMAs is represented by 4-5 cores from 4-5 zones of pure cell types as defined by two pathologists. Duplicate cores from the chosen zones were used for array fabrication so that all zones are represented in duplicate. Thus these TMAs are unusual in that they have 4−5×2 cores per case on the array. The TMAs are under continuous construction with the next phase to include 100 additional UCI cases so that the arrays available for the proposed study will exceed the present 272 case set. The prototype array at the 66 case stage have been utilized for the evaluation of several potential antibody by markers including Claudin I and Bcl-B (Krajewska et al. 2007; Krajewska et al. 2008).

C. 6. Colocalization. The studies of Krajewska et al. (Krajewska 2007; Krajewska 2008) utilized double antibody labeling of the same TMA section using anti-Claudin I and anti-cytokeratin in the double chromagen mode. For colocalization the two color were separated using a segmentation program developed by Aperio Technologies and represented individually and provide clear indication of the epithelial binding pattern of anti-Claudin-I. Pixel count and quantification of colorcalization as well as nonlocalized binding is readily possible although non specific binding for anti-Claudin-I is negligible in this example. The method is less easily generalized to three or more colors or to IF as yet and therefore is less versatile than the Thora system of Vala preferred for this application however it provides further illustration of our early experience in the methods proposed here.

Conclusions. Candidate gene expression levels for diagnosis and prognosis have been derived. Methods for the high throughput and quantitative assessment of labeling by corresponding antibodies are available. The wedding of this methods promises to provide the means of developing reference and assessment antibodies for new ICON-compliant clinical assays which solve significant unmet needs.

Phase I. Here we focus on attaining milestones that support the goal of demonstrating that reference antibodies and methods are available for the reliable and quantitative identification of cells of interest for use in Phase II, the systematic assessment of candidate biomarker antibodies for the development of panels for the multiplex determination of diagnosis and prognosis

Milestone 1. Develop an automated optimized imaging assay and SOP for prostate stroma and epithelial/tumor cells using three or more antibodies for immunohistochemistry and immunofluorescence.

Unstained sections of formalin-fixed paraffin-embedded prostate tumors, unstained sections of our prostate cancer TMAs and frozen sections of frozen prostate carcinoma-bearing tissues will be utilized. FFPE blocks will be taken from the extensive collection used for construction of the TMAs. Frozen tissues are available from the UCI SPECS program. Antibodies for the labeling of all epithelial structures, just tumor epithelium, and the fibroblast/myofibroblasts component of stroma will be optimized separately for all three tissue preparations. Screening studies will be carried out using chromagen labeling by indirect IHC using DAB for ease of visual monitoring and optimization will be extended to indirect IF.

Panepithelial labeling. Panepithelial labeling will be used as a reference to define candidate antibody biomarker labeling that colocalizes with bona fide epithelium in prostate cancer sections and therefore to derive a ratio of epithelial:nonepithelial labeling as a measure of specificity. Panepithelial labeling will be optimized for two antibodies and the best one of these used for all subsequent studies. Anti-high molecular cytokeratin (anti-HMW keratin; Dako clone 34βE12 mouse monoclonal anticytokeratin) will be used at the starting conditions that we have previously employed for the prostate cancer TMAs (Krajewski 2007). The antibody labels squamous, ductal and complex epithelia containing cytokeratins 1, 5, 10, and 14 (68, 58, 56.5′ and 50 kDa proteins).

A second anti-panepithelial antibody is AE3/AE4 (Dako AE3/AE4 MNF116 mouse monoclonal antihuman) which is in standard clinical use in the Pathology Department at UCI for the identification of epithelial components especially in the investigation of metastatic spread of carcinomas in distant tissues. The antibody labels multiple cytokeratins (65-67, 64, 59, 58, 56.5, 56, 54, 52, 50, 48 and 40 kDa cytokeratins) in either FFPE or frozen tissue.

Tumor epithelial cell labeling. Tumor epithelial cell labeling will be used as a reference to define the colocalization of labeling by candidate antibody biomarkers with bona fide tumor cells and therefore to derive the ratio tumor cell labling:non tumor cell labeling as a measure of specificity. Prostate cancer tumor epithelial cell labeling provides a more specific reference site for co-localization studies to be carried out in Phase II but is a challenging reference target owing to the limited number of antigens accepted as expressed in prostate cancer epithelial cells independent of the degree of differentiation or other histological properties such as Gleason score. We previously examined the expression pattern at the RNA level for a series of 55 tumors where expression could be resolved to the principal cells types (tumor epithelial cells, BPH epithelial cells, dilated cystic gland lining epithelium and stroma) which revealed that several classically expressed antigens such as PSMA (prostate specific membrane antigen), PAP (prostate acid phosphatase), and AMACR (a-methyl acyl CoA racemase) where significantly expressed at the RNA in nearly all tumor cells independent of grade and stage (Stuart et al. 2004). In this study we validated the protein expression was specific in seven representative cases (Stuart et al. 2004) using IHC.

Anti-AMACR is now in widespread clinical use for the identification of metastatic prostate cancer and has been reviewed extensively (e.g. Rubin 2004). In an analysis of anti-AMACR labeling of a prostate cancer TMA of 70 cases including “foamy” cell carcinoma with low expression of AMACR, labeling was detected in 91% percent of cases (Rubin 2004). Specificity and sensitivity were examined by quantitative receiver operator characteristic which yields an AUC was 0.9 (p<0.00001). These values are highly encouraging for the approach proposed here. It is not necessary to identify all prostate cancer cells but rather label a statistically valid sampling in order to assess, on this sample, the colocalization properties of candidate antibody biomarkers. Thus, a 91% labeling efficiency is very acceptable. We will employ the same commercial antibody and procedures as for Rubin et al. (Rubin 2004): mouse monoclonal anti-AMACR p504s (Zeta Corp., Sierra Madre, Calif.) at a starting dilution for optimization (see below) of 1:25. The optimization protocol to be used here encompasses the conditions of Rubin et al. (Rubin 2004). A major potential advantage of anti-AMACR is that the weak or absent labeling of normal epithelial components will facilitate quantification of nonspecific labeling (“noncolocalized labeling”) by candidate biomarker antibodies to be developed in Phase II.

Other potential tumor epithelial cell antibodies include anti-PSMA, anti-PSA, and anti-PAP. Antibodies to these products react with epithelium of normal and malignant cells. Anti-PSMA is extensively studied, is FDA approved (clone 7E11) for radiological detection of PCa metastases, labels nearly 100% of tumors in histological sections, and consistently label tumors at greater intensity that benign prostate epithelium (Chang 2004). We will optimize the labeling of FFPE, TMAs, and frozen sections test with our quantitative IF methods can exploit this property to distinguish tumor from benign labeling in comparison to anti-AMACR and visual scoring. We will utilize a mouse monoclonal anti-human PSMA (Dako clone 3E6).

Stroma cell labeling. “Stroma” as used here is a collective term consistent largely of fibroblasts, myofibroblasts and less proportion of vascular, neural, and other elements. Fibroblast and myofibroblasts labeling will be used as a reference to identify colocalization of stroma-binding candidate biomarker antibodies and to derive the ration of stroma:nonstroma labeling by the candidate antibodies. Widely accepted markers that may make suitable reference antibodies consist of anti-desmin, anti-vimentin, and smooth type α-actin and others (Castellucci 1996; Tuxhorn 2002; Ayala 2003; Tomas 2004; Ao 2006; Jiang 2007). We have previously utilized anti-desmin for the IHC analysis of prostate cancer (Stuart 2004). Considerable literature has accumulated indicating that Vimentin and smooth muscle type α-alpha vary in expression in PCa depending on the extent of epithelial-mesenchymal transformation and reactive stroma formation, two processes that correlate with aggression (Tuxhorn 2002; Ayala 2003:Hyanagisawa 2007; Yang 2008)). These phenomena appear to be proximal to the site of PCa. These markers therefore have the potential to delimit the “field” effects that are associated with differential gene expression of tumor-adjacent stroma. These observation correlate well with our observations that tumor-adjacent stroma contain numerous differentially expressed genes useful for diagnosis and for prognosis. Indeed, as noted, the mRNA levels of desmin and vimentin are significantly increased in stroma of our PCa samples compared to the epithelial components (Stuart et al. 2004). We plane, therefore, to optimize all three antibodies and determine their suitability as reference antibodies for stroma in general and tumor-adjacent stroma in particular. Previously characterized stroma reference antibodies include: anti-desmin mouse monoclonal antibody Dako clone D33 (Stuart 2004); anti-vimentin goat polyclonal sera cat. No. AB1620 from Chemicon (Temecula, Calif.) (Tuxhorn 2002); and anti-smooth muscle α-actin Dako clone IA4 (Tuxhorn 2002). For the development of stable renewable reagent sources it is highly desirable to work with monoclonal antibodies where source licensing can be organized. Therefore for anti-vimentin we will also examin mouse monoclona antibody from Dako, clone V9.

Optimization and SOP development. The primary antibodies will be applied using an automated immunostainer (DAKO Universal Staining System) and employing the Envision-Plus-horseradish peroxidase system (DakoCytomation, Inc.) secondary labeling system for DAB. FFPE sections will be deparaffinized by xylene overnight followed by microwave treatment and 0.4 power for 30 min. in a 6.0-pH citrate buffer. No enzymes or other “antigen retrieval” processes will be applied here or any of the labeling conditions considered here in order to minimize the variables required in developing panels of multiple antibodies with compatible protocols (Phase II). Sections will be pre-treated with normal mouse serum for 40 min. and washed in PBS with automated stirring three times. For optimization, primary antibodies will be applied at room temperature for 40 min in two-fold serial dilution from 1:30 through 1:960 or higher dilutions if practical. The optimal titre (as well as the preceding and following titre value) as judged by visual appearance (D. Mercola, F.C.A.P.) of specific labeling intensity to background labeling intensity will be re-tested on sections with increased deparaffinization steps (see IF procedure) including an over night baking step and reduced as well as extended microwaving to check for an improvement in signal to background labeling intensity. Finally, the time and temperature of application of the primary antibody will be optimized by comparing exposure to primary antibodies for 2 h and 24 h at room temperature and 24 at 4 deg. C.

These steps will be applied to both FFPE and frozen sections of fresh tissue. In the case of fresh tissue, we will utilize samples that have been cryopreserved in liquid nitrogen from the time of initial freezing. All samples for the UCI SPECS project are obtained directly from the O.R. and processed by an expedited surgical pathology grossing procedure. Sample for research are taken from tissue adjacent to the grossly identified tumor site or, for “remote” tissue control samples, taken from the contralateral prostate. Tracking sheets are maintained on all samples giving the elapsed time from the O.R. to freezing. Representative samples are used for RNA q.c. as an indication of preservation by analysis of total RNA using an Agilent Bioanalyzer which indicates high levels of preservation in over 95% of samples. Frozen sections will be prepared from these tissues directly from the frozen state without thawing. The sections will be fixed for 60 sec. in 95% methanol or 100% acetone or 70% EtOH all at −22 deg. C., air-dried, and used directly for antibody optimization.

TMA confirmation. Optimized labeling protocols developed on FFPE sections will be tested by application to our TMA with 272 cases including cores of tumor-adjacent and remote stroma. Labeling of the TMAs will provide information of the generality of labeling across cases and the reproducibility of specific labeling for tumor and stroma. To insure that optimization has been achieved for the TMAs, the last steps of the optimization procedure will be repeated using the TMA sections, i.e. the application of primary antibody using the three best titre values and the following steps. Progress will be monitored by visual inspection of the DAB labeled slides (D. Mercola, F.C.A.P). Optimal conditions will be judged by the most cases of the TMA that reflect the desired criteria of the greatest differential expression between target cell type with “background” intensity. All informative slides will be stored in a temperature controlled laboratory for scanning and quantitative assessment of variability, accuracy, and reproducibility assessment of Milestones 3 and 4.

Immunofluorescence. Immunofluoresce is the intended method of choice owing to the much higher dynamic range and sensitivity of antigen detection. Indeed, we anticipate that primary antibodies can be extended to high titres by factors of 10× or more. The major challenge is selection of conditions that minimize “background” or “autofluorescence”. Background fluorescence can be minimize by using fluorophores with long wavelength emission (>500 nm), use of sections with rigorous deparaffinization procedures (i.e. the overnight deparaffinzation xylene treatment and used of prolong baking of unstained FFPE sections, above), use of pretested acid washed slides and coverslipping reagents, and use of a configuration of the robotic microscope with optical filter wheel located before the monochrome CCD camera. These methods have been optimized previously (Rabinovich 2006). The characterized fluorophore-conjugated secondary antibodies to be used previously that will be applied here are: Texas Red-labeled goat anti-mouse (catalog number 115-075-146, Jackson Laboratories, Bar Harbor, Me.) and Alexa Fluor 488-labeled goat anti-mouse (catalog number A21121, Molecular Probes, Eugene, Oreg.). These reagents can be used at dilutions in the range 1:1,000 to 1:10,000. The optimum concentration will be determined for sections of our TMAs.

Visual assessment of optimum conditions require counter staining. Sections will be stained with DAPI (Molecular Probes, Eugene, Oreg.) at 75 ng/ml (in 10 mM TRIS, 10 mM EDTA, 100 mM NaCl) for 45 mM prior to sealing with coverslips. Visual assessment will be carried out by J. Price and D. Mercola.

Milestone 2. Storage and visualization will utilize exiting technology of the Vala Sciences Inc. system. All data will also be placed in a free database that is DICOM compliant.

In this project the bulk of data collection, storage, and analysis will be by the Vala Science robotic scanning microscope and associated software and storage capacity. As reviewed here (Preliminary Studies), Throra and associated software for data acquisition, analysis and storage are advanced. These are most completely described in the specialty publications of Rabinovich et al. (Rabinovich 2006) and Prignoshima et al. (Prigoshina 2007). Moreover Proven Inc. and Vala Sciences Inc. are committed to the development of completely DICOM complaint storage and data sharing (http:/www.sph.sc.edu/comd/rorden/dicom.html). The primary data of the assay proposed here, a multiplexed antibody assay utilizing indirect IF, will consist of a spectral stack of multiple color images of histological section of biopsies or postprostatectomy tissue sections together with standard hematoxylin and eosin stained sections of the same section used for IF labeling. Such images represent a novel data set for diagnosis and prognosis without direct precedent in the DICOM standard. Since Phase II is focused on product development for diagnosis and prognosis in the CLIA reference lab setting, Vala Science Inc. is very interested in developing a DICOM-compatible format for the storage and transmission of primary tissue images. It is planned to develop a demonstration format using DICOM heading and other features in analogy of other imaging systems.

Milestone 3. SOPs will be developed for specimen collection, processing, and stability of the cell types in the imaging assay.

SOPs for the acquisition of tissues and blocks have been developed by the UCI SPECS program and are maintained as date pdf files and in an SOP workbook. These SOPs describe procedure for informed-consent based patient recruitment at all participating sides and methods of tissue collection at O.R rooms, expedited processing and storage together with diagrammatic illustrations of dissection procedures and additional tracking forms for each specimen. All procedures are UCI 1RB-approved and HIPPA-compliant. In addition the UCI SPECS program maintains “shadow charts” for all recruited patients including the signed witness informed consent, tracking sheets, and CRFs of baseline clinical data together with source documentation of all values recorded in the SPECS data base. The data base is maintained on a devoted server hosted by a participating institute, the Sidney Kimmel Cancer Center of San Diego, in a locked server room under the control of the SKCC IT department. The server is accessed remotely via a password protected web-based portal by approved clinical coordinators and the data base manager. All personnel are UCI employees. The SOPs will be incorporated into the SOPs generated for phase I of this project.

SOPs describing the optimized procedures and reagents of Milestone 1 will be developed as final conditions are determined. The methods for the fabrication of the TMAs will be included. These will include methods for periodic testing to insure stability of the labeling results. The current TMAs contain cores of fixed cultured prostate cells including standard tumor cells (LnCAP, PC3, DU145, M12) and normal immortalized cells (RWPE1, p69) will will be used to record quantified labeling intensity. Upon the completion of Milestone 1, multiple section of the TMA block containing cell cores will be prepared as a master lot for periodic qc and for standardizing new lots of renewable reagents. These procedures will be included in the SOPs.

It is a major goal of phase II to initiate a prospective validation program using newly recruited clinical patients and UCI and applying the multiplex panel to research biopsies and post surgery tissue specimens in the CLIA lab of the molecular pathology core of the UCI Department of Pathology and Laboratory Medicine. In anticipation of this study, All SOPs, master lot preparations, and DICOM-capatible image storage will be coordinated with CLIA requirements of this laboratory.

### Specific Aim 1: Generation and Initial Characterization of Predictive Antibodies.

1. Acquisition of 25 candidate antibodies against antigens identified as predictive of prostate cancer progression or recurrence based upon the preliminary studies (Section C).
2. Western analysis and IHC analysis of 25 candidate antibodies in order to confirm cell-specific expression and specificity.
3. Prioritize antibodies for testing on TMAs (Aim 2) based upon the intensity of cell-specific tissue labeling, the specificity as judged by the observation of predominate binding to a protein of the predicted molecular weight in Western analysis, and sensitivity as judged by percent of cells of the expected type in IHC labeled tissue sections.

### Specific Aim 2: Validation of Prostate Cancer Predictive Antibodies on Tissue Microarrays (TMAs).

1. IHC analysis of 6-10 prioritized candidate antibodies on TMAs constructed from 254 annotated clinical prostate cancer cases. Analysis will consist of the determination of manual “immunoscores” by three pathologists.
2. Kaplan-Meier analysis comparison of immunoscores with clinical outcomes for 5-8 candidate antibodies.
3. Prioritize antibodies for clinical development based upon sensitivity, specificity, and accuracy as determined from the Kaplan-Meier analysis of Aim 2-2 and the magnitude of the differential expression between non-recurrent and recurrent cases. Antibodies also will be prioritized by their ability to contribute to a classifier” panel of antibodies, i.e. the minimum number of antibodies that encompass the “diversity” of the 254 cases. The measure of “encompassing diversity” will be the number of cases whose survival category is uniquely recognized by that antibody. These criteria insure the development of the smallest antibody panel necessary. Since the TMAs are fabricated from cases entirely independent of those used for MLR, confirmation of differential express here extends the generality of the biomarker antibodies and, ipso facto, extends the biomarkers to the protein level. The panel of antibodies successful at this level will represent both significant changes in tumor cell expression between recurrent and nonrecurrent cases and will include tumor microenvironment changes in between recurrent and nonrecurrent cases, a key ingredient in building a robust classifier.

### Specific Aim 3: Automated and Improved Quantification of TMA Readout.

1. Quantify and validate the two-color separation method by (i) quantification of pixel intensity of test antibodies only at the locus pixels of specific cell types such as all epithelium or all prostate cancer as defined by cell-specific markers such as anti-cytokeratin or anti-Amacr (Aim 2-1) and (ii) validate the quantification approach by correlation with visual immunoscores. Pearson and Spearman correlation coefficients will be determined, together with probabilities of the correlation coefficients as well as the degree of relatedness (slope) of visual and quantified scores.

### D. METHODS

Specific Aim 1: Generation and initial characterization of predictive antibodies to epithelial and stroma tumor antigens. Antibodies against known prostate cancer antigens and against putative prostate cancer biomarkers identified by gene expression analysis will be obtained from commercial sources and characterized using Western blotting and immunohistochemistry. Candidate antibodies that demonstrate the ability to detect discrete proteins on Western Blots prepared from fresh prostate tissue samples (stroma or tumor) and the ability to differentially label cell types in paraffin-embedded prostate cancer tissue sections will identified. Their ability to predict clinical outcome will be tested in specific aim 2.

D.1.a. Description of Antibodies

Commercial antibodies will be purchased, if available. Other antibodies will be generated (Lampire Biologicals, San Diego, Calif.). Numerous antibodies used in our separate projects have been developed in cooperation with Lampire Biologicals [50, 68-74].

Three classes of antibodies will be tested:

1. Antibodies that label prostate tumor cells, normal epithelium, or stromal cells to be used as internal standards will be used to identify specific cell-types within prostate tissue samples. Those on hand of particular importance for the identification of epithelial components include anti-high molecular weight cytokeratin (HMW cytokeratin), anti-PSA, anti-PAP, anti-PSMA, and anti-Amacr. Those intended for the identification of stroma include anti-Desmin and anti-smooth muscle alpha actin (Anti-ACTA). We have optimized all of these for use with FFPE tissue sections and described results in previous studies [18, 67].

2. Antibodies against potential prognostic markers identified by gene expression analysis. Twelve commercially available antibodies against predicted antigens have been obtained and screened using standard sections of FFPE prostate cancer tissue blocks. Five of these antibodies are very promising for detailed characterization as proposed here. Antibodies that are not available or exhibit poor labeling or background properties in screening will be commissioned de novo as described below.

3. The selection and screening of additional antibodies will be prioritized by starting with antibodies to gene products that exhibit the largest differential labeling (largest difference in immunoscore or normalized pixel intensity) between nonrecurrent and recurrent prostate cancer cases. As noted above, approximately half of the antibodies screened so far do exhibit excellent signal to background properties on test sections of FFPE prostate cancer.

D.1.b. Criteria for Inclusion of Antibodies for TMA Analysis Will Include: Path to Monoclonal Antibody Production.

1. Antibodies are suggested by the results of MLR (Preliminary Data, Section C1).

Candidate antibodies first will be vetted by Western analysis to test for the detection of antigen of correct molecular weight in prostate tumor tissue extracts or alternative molecular weights previously reported as prostate cancer-variants. Previous experience [18] has revealed that an important factor in meeting these criteria is knowledge of the origin of the antigen. The linear regression results identify probe sets of Affymetrix GeneChips which correspond to precise genes and introns of genes. Commercial antibodies against recombinant proteins or large fragments of proteins likely correspond to the identified gene product and so are useful for testing whether genes of probe sets are expressed at the protein level. Similarly, commercial antibodies against highly pure native proteins of a carefully characterized molecular weight that agrees with that expected value on the basis of the Affymetrix-predicted gene product also may be expected to be confirmed by Western analysis. However, antibodies produced against proteins purified from natural sources may contain alternative spliced products and/or other gene family member proteins as well as closely related proteins or fragments that are difficult to separate during purification may lead to antibodies reactive to a range of molecular weights with an unclear relationship to the gene product corresponding to the Affymetrix probe set. Monoclonal antibodies against recombinant or synthetic peptides more often meet the need for single gene product specificity and will be preferred. In addition monoclonal (mouse, rat) define a potentially renewable resource that may be contracted as a stable supplier of test kit reagents. Therefore, all polyclonal antibodies characterized here for inclusion on the final antibody classifier will replicated by the commissioned preparation of the corresponding monoclonal antibody as part of phase II.

2. Consistent and robust IHC signal of antigens from formalin-fixed and paraffin-embedded (FFPE) tissue. TMAs provide a major advantage in that the fraction of cases exhibiting increased or decreased IHC signal may be quantified readily. In order to develop an assay with maximum reproducibility, methods that minimize reliance on “antigen retrieval” strategies will be adopted. This will select for robust antibodies capable of recognizing antigens on archived samples.

3. Consistent and robust IHC signal of antigens from archived (>10 years) FFPE tissue. IHC labeling intensity for each antibody will be correlated with the age of the sample on the TMA. An advantage of our TMAs is the presence of cases from 2 to 19 years old.

4. Cell-specific labeling. Cell identity (normal epithelium, stroma, BPH) will be determined by manual inspection or staining with cell-specific antibodies. IHC intensity for each antibody will be immunoscored for staining intensity and cell specificity as described below (Sections D.2.c. or D.3.b.)

D.1.b. Tissue source for Western blotting. Tissues will be obtained from the UCI SPECS prostate project tissue bank This is a resource of the NIH-supported UCI SPECS prostate project. Prostate samples were obtained from patients (UCI) that were preoperatively staged as having organ-confined prostate cancer. Institutional Review Board-approved informed consent for participation in this project was obtained from all patients. Tissue samples were collected in the operating room, and specimens were immediately transported to institutional pathologists who provided fresh portions of grossly identifiable or suspected tumor tissue and separate portions of uninvolved tissues that were excess to patient care needs (surgical pathology staging and confirmatory diagnosis). All excess tissue was snap frozen upon receipt and maintained in liquid nitrogen until used for frozen section preparation at −22° C. Fifty five percent of all cases collected in this series contained histologically confirmed tumor tissue. Portions of frozen samples enriched for tumor, stroma, BPH, and dilated cystic glands are identified by examination of frozen sections. When suitable tissues are identified, thick frozen sections of 20 microns are collected in separate Eppendorf tubes for lysis and Western analysis.

Additionally, the ability of antibodies to visualize antigens of correct MW on Western blots from tissue extracts established from a panel of human prostate cell lines will be determined. This panel will include androgen resistant prostate cancer cells (PC3, DU145), androgen sensitive prostate cancer cells (LnCAP), primary immortalized RWPE-1 epithelial cells. Cancer cells of alternative derivation (lung, breast, colon), and several normal cell lines (fibroblasts, myoblasts) (ATCC) (these cells have also been applied to the TMAs as sections of formalin-fixed cell pellets).

D.1.c. Western Blotting

Tissues or cultured cells will be lysed in either 1×Laemmli solution lacking bromophenol blue or in RIPA buffer (0.15 mM NaCl/0.05 mM Tris.HCl, pH 7.2/1% Triton X-100/1% sodium deoxycholate/0.1% sodium dodecyl sulfate) containing protease inhibitors including the caspase inhibitors 100 μM Z-Asp-2.6-dichlorobenzoyloxymethyl-ketone (Bachem) and Z-Val-Ala-Asp-fmk (Calbiochem). Total protein content will be quantified by either the Bradford or bicinchoninic acid methods (Pierce). SDS/PAGE and immunoblotting with enhanced chemiluminescence-based detection (Amersham Pharmacia) will be performed [50, 69-71].

Antibody reactivity will be semiquantified by comparison of reaction intensity of tissue and cellular extracts with extracts of prostate cancer cells (PC3, LNCaP) and negative control cells (bacterial cultures and female normal breast epithelial cells, MCF10A) of known total protein mass.

### D.1.d Immunohistochemistry.

Our methods for optimization and detection of antibody labeling have been described extensively [50, 68-74]. Briefly, the cell specificity of the identified antibody for normal and malignant prostate tissue will be tested by comparing the binding patterns on a series of normal and malignant prostate tissue specimens. FFPE tissue sections (5 μm) will be deparaffinized, microwave-heated, and immunolabeled by indirect staining using either a conjugated secondary antibody for avidin-biotin complex formation with horseradish peroxidase (HRP) using the Vecta labeling reagents (Vector Laboratories) followed by addition of diaminobenzidine (DAB) for colorimetric detection or the Envision-Plus-HRP system (Dako) with a Dako Universal Staining System. A range of antibody concentrations will be tested to optimize signal detection and specificity. For all tissues examined, the immunostaining procedure will be performed in parallel by using either preimmune serum (polyclonals) to verify specificity, or the antiserum reabsorbed with 5-10 μg/ml of synthetic peptide or recombinant protein immunogen where available. Positive controls for cell-type specificity will be determined by staining sections with a “cocktail” of antibodies directed against pan-cytokeratin (Sigma) to identify epithelial cells and antibodies against Desmin, alpha-smooth muscle actin, or prolyl-4-hydroxylase to identify stromal cells

Specific Aim 2: Validation of prostate cancer predictive antibodies on tissue microarrays (TMAs). Our TMAs have been constructed from archived prostate tissue samples with known clinical outcomes from SKCC and UCI. IHC staining will be performed using antibodies developed in Specific Aim 1. IHC staining levels will be immunoscored (below) and compared to clinical outcomes by Kaplan-Meier analysis. Significance of discrimination of survival groups will be determined by the Cox Proportional Hazards model.

Visual determination is carried out by three pathologists (SK, MK, and DAM) and averaged. Candidate antibodies demonstrating the greatest sensitivity, specificity, and accuracy for the prediction of clinical outcome by the Kaplan-Meier criterion will be selected for the antibody panel for prognostic validation of clinical samples in Phase II.

D2.b. Immunohistochemistry on TMAs. Immunohistochemistry on TMAs will be performed as described previously [50, 69-71] and above (Section D.1.d.)

D.2.c. Immunoscoring of TMA Readouts

Immunoscores are determined visually and are formed as a product of the percent of a given cell type that is positive 1-100 percent) times the intensity on a three point scale yielding a range of values from 1-300 [68-70, 72, 73]. For the three-point scale intensity is j judged as 0, negative; 1+, weak; 2+, moderate; and 3+, strong [70]. Samples will be additionally scored for percentage of immunopositive malignant cells, estimating the percentage in increments of 10% (0%, 10%, 20%, 30%, and so on) from a minimum of five representative medium-power fields. The scoring will then be based on the percentage of immunopositive cells (0 to 100) multiplied by staining intensity score (0/1/2/3), yielding scores of 0 to 300. Scoring is conducted in a joint session of the three pathologists utilizing the original glass slides and a multihead microscrope in order to insure identical viewing times and field exposures. The reproducibility and agreement among pathologists following this format has been assessed [18] and immunoscoring using the above scales has been used in several studies [50, 69-71].

D.1.d. Statistical Analysis

Data will be analyzed using the JMP Statistics software package (SAS Institute, Cary, N.C.), and STATISTICA Software (StatSoft, Tulsa, Okla.). Comparisons of antibody immunostaining data with patient survival will be made using the Cox proportional hazards model and the comparison of Kaplan-Meier survival curves. An unpaired t test method was used for correlation of immunoscores with the available patient data. All statistical methods will be supervised by our biostatistician, Zhenyu Jia, consultant for Phases I and II of this project (see Biosketch, Z. Jia and letter).

Antibody performance will be judged by conventional operating characteristics (accuracy, sensitivity, and specificity) but also by criteria that produce the smallest panels that maximizes the percent of cases of the TMA accurately discriminated as aggressive or nonagressive by survival and other criteria. This is an important consideration, as a true classifier panel should contain biomarkers effective with cases that other biomarkers may be insensitive to, i.e. cover the diversity of prostate cancer. Thus, individual antibodies will be scored by the number of cases unique classified with very large or very small odds ratios that other antibodies fail to distinguish (i.e. the number of unique cases accurately classified). These criteria further insure that the minimum number of antibodies to discriminate all amendable cases of the TMA will be formed.

Specific Aim 3: Automation and improved quantification of TMA readout. The discriminatory power and the rate of characterization of the prognostic antibodies identified in Specific Aim 2 may be improved using image analysis that provides for quantitative determination of antibody labeling intensity. Rapid scanning, digitization, and the use of a newly developed algorithm for two-color separation are established at the BIMR largely as the developmental work of one of the applicants (SK). Digitized IHC labeled prostate TMA are maintain on a server located at the BIMR and accessible by all participants via a secure portal (https://scanscope.burnham.org/Login.php). This greatly facilitates the monitoring of IHC results and planning of next steps and immunoscoring sessions. UCI SPECS pathologists utilize high resolution line scanned H and E and IHC images of this site for immunoscoring of other projects and confirmed the histological features of the TMAs such as Gleason scores, presence of PIN, etc. This technology allows for automated quantification of cell-specific antibody staining of TMA samples without reliance on “shape recognition” or manual inspection to determine cell-type. This technology will be tested using the panel of prognostic antibodies developed in the first two specific aims.

Specific Aim 3: Automation and Improved Quantification of TMA Readout. D.3.a. Double Labeling.

Double labeling places constraints on the combination of standard (anti-PSMA, anti-AMACR, and anti-cytokeratin) and candidate antibody combinations owing to the need to use secondary antibodies for the development of two different chromagens. The methods that we have previously used for double labeling (Krajewski 2007; Krajewska 2008) will be followed closely. In general candidate antibodies will be derived from rabbit sera. Indirect IHC using biotin labeled anti-rabbit IgG will be applied for development of DAB (3,3¶-diaminobenzidine chromagen, DAKOCytomation; brown). Mouse monoclonal antibodies to AMACR, PSMA, or cytokeratin will be identified by addition of biotin-labeled anti-mouse for development of the black SG precipitate (Serotec; SG chromagen, Vector Lab., Inc.; black). No or very light counter staining with Nuclear Red (DAKOCytomation) will be applied

D.3.b. Validation of prostate cancer predictive antibodies on tissue microarrays (TMAs). Color unmixing has been validated for sections labeled with hematoxyln and DAB (Preliminary Data). As noted, actual isolation of subsets of pixels that co-localize with epithelial or tumor cells is a milestone of Phase I. Validation will be extended to DAB and SG double labeled sections and to colocalized integrated and normalize pixel values. For this purpose it is important to note that visual scores are traditional obtained as the product of the intensity of labeling (on a 0 to 3+scale) times the percent of tumor or epithelial cells that exhibit positive labeling. Here both factors will be used to validate co-localization. A test system utilizing a polyclonal anti-AMACR (DAB) and monoclonal anti-cytokeratin (SG) alone and in combination will be applied to both the tumor TMA and to the BPH TMA. First, analogous to the hematoxyln-DAB system, deconvolution results (reconstructed DAB image and reconstructed SG image) for the combination labeling will be compared to individual labeling (ground truth). These tests will define the accuracy as percent error+/−standard deviation for each chromagen. Second, colocalized pixel sums for AMACR labeling as a “standard” for binding to a high percentage of tumor cells will be determined. This is the sum of pixel intensity for DAB at pixels positive for SG. The pixel sum for DAB will be normalized to SG for all cases to correct for the variable amount of total epithelium on each core. The normalized sums are expected to be maximal for tumor sections where AMACR expression is commonly positive in most cells of most tumors but to exhibit minimum overlap in cases of BPH. Indeed simple thresholding may succeed defining a single value that best separates average tumor from average BPH. This may be expected since AMACR labeling will be applied based on optimization of tumor sections. Third, visual score by two pathologists (S. Krajewski and D. Mercola) will be acquired for all the single-antibody (DAB or SG) labeled TMAs. The results of spectral unmixing for DAB and SG will be compared to visual scoring for these chromagens as for the previous studies. Finally, the normalized DAB pixel sum is expected accurately correlate with the percent tumor cell component determined by the pathology and especially to correlate with the ration of percent DAB positive tumor cells over percent positive SG cytokeratin cells Thus, globally we predict:

$$\frac{\begin{matrix}Case average co \text{-} localization \\ pixel sum for A M A C R ( D A B ) \end{matrix} }{Case average pixel sum for Cytokeratin ( S G )} ∼ \frac{Case average vis . \% positive A M A C R}{Case average vis . \% positive Cytokeratin}$$

On a case by case basis plots of normalize DAB/SG vs. percent DAB positive/percent SG are predicted to have a high Pearson correlation with a slope ˜1 and error similar to the preliminary Results of <10%. Validation of spectral unmizing for this chromaphore system will provide a major milestone of Phase I and means of automated antibody biomarker screening of Phase II.

Candidate stroma biomarker antibodies will be treated in a converse fashion. Mutually exclusive pixel sums (all pixels other than cytokeratin-positive pixels) will be integrated. This guarantees that epithelial components. These values will be normalized to the nonepithelial pixel sum intensity for a trichrome stain of the TMA using a second spectral unmixing calculation to identify connective tissue component (blue).

### Antibodies

We are aware that the quantification method being developed here has numerous additional standardization issues. It is entirely dependent on the properties of reference antibodies to define “cell-type”. Antiamacr is in wide clinical use for the identification of prostate tumor cells in non prostate tissue in the presence of other components including glands. Nevertheless it is not unchallenged and “negative” results have been noted to occur for up to 30% of prostate cancer cells [76-81]. Thus pixels identified by these criteria may only “sample” a large proportion of tumor cells. This may be acceptable unless particular classes of tumor cells such as those expressing genes correlating with, say, rercurrence, are preferentially negative. It will be important to utilize other criteria such as visual inspection by trained pathologist and the use of other faithful tumor cell markers reveal significant bias.

We have identified a large panel of genes that are preferentially expressed by prostate tumor cells [18]. In addition, standard alternatives such as antiPSA and antiPSMA may be compared to determine labeling deficiency by antiAmacr.

We have chosen to concentrate on the use of monoclonal antibodies for these studies as they generally display higher specificity and consistency compared to polyclonals and are therefore better adapted to commercialization into clinical development. Polyclonal antibodies are commercially available and might prove to be more sensitive in FFPE tissues, and therefore may be explored. Commissioned monoclonal antibodies are amenable to clear definition of ownership and path to market.

Many antibodies against prostate cancer tissues are commercially available. However, antibodies against important biomarkers that are not currently commercially available or that fail to meet quality control specified in specific aim 1 will be made using peptide antigens (Lampire Biologicals, San Diego, Calif.) as for previous studies [50, 68-74].

Finally an important challenge in Phase II will be the combining of multiple antibodies with possible individual optimization protocols to a single tissue section. If this can not be achieved conveniently, i.e. without serial application, the panel will be applied on multiple slides using 2-3 different antibodies of the panel per slide. Although less convenient, the use of two or possible three serial sections of patient biopsy tissue does materially effect the ability to derive prognosis from our predictive antibody panel.

### E. BIBLIOGRAPHY

1. Flaig, T. W., et al., Conference report and review: current status of biomarkers potentially associated with prostate cancer outcomes. J Urol, 2007. 177(4): p. 1229-37.
2. Steuber, T., P. Helo, and H. Lilja, Circulating biomarkers for prostate cancer. World J Urol, 2007. 25(2): p. 111-9.
3. Reynolds, M. A., et al., Molecular markers for prostate cancer. Cancer Lett, 2007. 249(1): p. 5-13.
4. Lilja, H., D. Ulmert, and A. J. Vickers, Prostate-specific antigen and prostate cancer: prediction, detection and monitoring. Nat Rev Cancer, 2008. 8(4): p. 268-78.
5. Stephan, C., et al., PSA and new biomarkers within multivariate models to improve early detection of prostate cancer. Cancer Lett, 2007. 249(1): p. 18-29.
6. Loeb, S, and W. J. Catalona, Prostate-specific antigen in clinical practice. Cancer Lett, 2007. 249(1): p. 30-9.
7. Loeb, S, and W. J. Catalona, Early versus delayed intervention for prostate cancer: the case for early intervention. Nat Clin Pract Urol, 2007. 4(7): p. 348-9.
8. Graif, T., et al., Under diagnosis and over diagnosis of prostate cancer. J Urol, 2007. 178(1): p. 88-92.
9. Loeb, S., et al., Risk of prostate cancer for young men with a prostate specific antigen less than their age specific median. J Urol, 2007. 177(5): p. 1745-8.
10. Steuber, T., et al., Risk assessment for biochemical rercurrence prior to radical prostatectomy: significant enhancement contributed by human glandular kallikrein 2 (hK2) and free prostate specific antigen (PSA) in men with moderate PSA-elevation in serum. Int J Cancer, 2006. 118(5): p. 1234-40.
11. Nam, R. K., et al., Assessing individual risk for prostate cancer. J Clin Oncol, 2007. 25(24): p. 3582-8.
12. May, M., et al., Validity of the CAPRA score to predict biochemical rercurrence free survival after radical prostatectomy. Results from a european multicenter survey of 1,296 patients. J Urol, 2007. 178(5): p. 1957-62; discussion 1962.
13. Bibikova, M., et al., Expression signatures that correlated with Gleason score and relapse in prostate cancer. Genomics, 2007. 89(6): p. 666-72.
14. Henshall, S. M., et al., Survival analysis of genome-wide gene expression profiles of prostate cancers identifies new prognostic targets of disease relapse. Cancer Res, 2003. 63(14): p. 4196-203.
15. Quinn, D. I., S. M. Henshall, and R. L. Sutherland, Molecular markers of prostate cancer outcome. Eur J Cancer, 2005. 41(6): p. 858-87.
16. Henshall, S. M., et al., Zinc-alpha2-glycoprotein expression as a predictor of metastatic prostate cancer following radical prostatectomy. J Natl Cancer Inst, 2006. 98(19): p. 1420-4.
17. Stephenson, R. A., et al., Metastatic model for human prostate cancer using orthotopic implantation in nude mice. Journal of the National Cancer Inst, 1992. 84: p. 951-957.
18. Stuart, R. O., et al., In silico dissection of cell-type-associated patterns of gene expression in prostate cancer. Proc Natl Acad Sci USA, 2004. 101(2): p. 615-20.
19. Richardson, A. M., et al., Global expression analysis of prostate cancer-associated stroma and epithelia. Diagn Mol Pathol, 2007. 16(4): p. 189-97.
20. Stephenson, A. J., et al., Integration of gene expression profiling and clinical variables to predict prostate carcinoma rercurrence after radical prostatectomy. Cancer, 2005. 104(2): p. 290-8.
21. Denmeade, S. R., et al., Dissociation between androgen responsiveness for malignant growth vs. expression of prostate specific differentiation markers PSA, hK2, and PSMA in human prostate cancer models. Prostate, 2003. 54(4): p. 249-57.
22. de la Taille, A., et al., Hormone-refractory prostate cancer: a multistep and multi-event process. Prostate Cancer and Prostatic Diseases, 2001. 4: p. 204-212.
23. Yu, X., et al., The association between total prostate specific antigen concentration and prostate specific antigen velocity. J Urol, 2007e. 177(4): p. 1298-302; discussion 1301-2.
24. Loeb, S., et al., Use of prostate-specific antigen velocity to follow up patients with isolated high-grade prostatic intraepithelial neoplasia on prostate biopsy. Urology, 2007. 69(1): p. 108-12.
25. Loeb, S., et al., Prostate specific antigen velocity threshold for predicting prostate cancer in young men. J Urol, 2007. 177(3): p. 899-902.
26. Gong, M. C., et al., Prostate-specific membrane antigen (PSMA)-specific monoclonal antibodies in the treatment of prostate and other cancers. Cancer Metastasis Rev, 1999. 18(4): p. 483-90.
27. Elgamal, A. A., et al., Prostate-specific membrane antigen (PSMA): current benefits and future value. Semin Surg Oncol, 2000. 18(1): p. 10-6.
28. Recker, F., et al., Human glandular kallikrein as a tool to improve discrimination of poorly differentiated and non-organ-confined prostate cancer compared with prostate-specific antigen. Urology, 2000. 55(4): p. 481-5.
29. Raaijmakers, R., et al., hK2 and Free PSA, a Prognostic Combination in Predicting Minimal Prostate Cancer in Screen-Detected Men within the PSA Range 4-10 ng/ml. Eur Urol, 2007.
30. Paliouras, M., C. Borgono, and E. P. Diamandis, Human tissue kallikreins: the cancer biomarker family. Cancer Lett, 2007. 249(1): p. 61-79.
31. Nam, R. K., et al., Variants of the hK2 protein gene (KLK2) are associated with serum hK2 levels and predict the presence of prostate cancer at biopsy. Clin Cancer Res, 2006. 12(21): p. 6452-8.
32. Diamandis, E. P. and G. M. Yousef, Human tissue kallikreins: a family of new cancer biomarkers. Clin Chem, 2002. 48(8): p. 1198-205.
33. Perambakam, S., et al., Induction of Tc2 cells with specificity for prostate-specific antigen from patients with hormone-refractory prostate cancer. Cancer Immunol Immunother, 2002. 51(5): p. 263-70.
34. McDevitt, M. R., et al., An alpha particle emitting antibody ([213BI]J591) for radioimmunotherapy of prostate cancer. Cancer Res, 2000. 60(21): p. 6095-100.
35. Steuber, T., et al., Free PSA isoforms and intact and cleaved forms of urokinase plasminogen activator receptor in serum improve selection of patients for prostate cancer biopsy. Int J Cancer, 2007. 120(7): p. 1499-504.
36. Wang, X., et al., Autoantibody signatures in prostate cancer. N Engl J Med, 2005. 353(12): p. 1224-35.
37. Stephan, C., et al., Three new serum markers for prostate cancer detection within a percent free PSA-based artificial neural network. Prostate, 2006. 66(6): p. 651-9.
38. Miyake, H., I. Hara, and H. Eto, Prediction of the extent of prostate cancer by the combined use of systematic biopsy and serum level of cathepsin D. Int J Urol, 2003. 10(4): p. 196-200.
39. Leman, E. S., et al., EPCA-2: a highly specific serum marker for prostate cancer. Urology, 2007. 69(4): p. 714-20.
40. Jiang, Z., et al., Discovery and clinical application of a novel prostate cancer marker: alpha-methylacyl CoA racemase (P504S). Am J Clin Pathol, 2004. 122(2): p. 275-89.
41. Hara, I., et al., Serum cathepsin D and its density in men with prostate cancer as new predictors of disease progression. Oncol Rep, 2002. 9(6): p. 1379-83.
42. Bradford, T. J., X. Wang, and A. M. Chinnaiyan, Cancer immunomics: using autoantibody signatures in the early detection of prostate cancer. Urol Oncol, 2006. 24(3): p. 237-42.
43. Wang, Y., et al., The challenge of developing predictive signatures for the outcome of newly diagnosed prostate cancer based on expression analysis and genetic changes of tumro and non-tumor cells, in 2007 American Association for Cancer Research Annual Meeting. 2007: Los Angeles, Calif.
44. Koziol, J. A., et al., The Wisdom of the Commons: Ensemble Tree Classifiers for Prostate Cancer Prognosis. Bioinformatics, 2008.
45. Datta, M. W., et al., The role of tissue microarrays in prostate cancer biomarker discovery. Adv Anat Pathol, 2007. 14(6): p. 408-18.
46. Diallo, J. S., et al., NOXA and PUMA expression add to clinical markers in predicting biochemical rercurrence of prostate cancer patients in a survival tree model. Clin Cancer Res, 2007. 13(23): p. 7044-52.
47. McDonnell, T. J., et al., Biomarker expression patterns that correlate with high grade features in treatment naive, organ-confined prostate cancer. BMC Med Genomics, 2008. 1: p. 1.
48. Prowatke, I., et al., Expression analysis of imbalanced genes in prostate carcinoma using tissue microarrays. Br J Cancer, 2007. 96(1): p. 82-8.
49. Ayala, G. E., et al., Stromal antiapoptotic paracrine loop in perineural invasion of prostatic carcinoma. Cancer Res, 2006. 66(10): p. 5159-64.
50. Krajewska, M., et al., Claudin-1 immunohistochemistry for distinguishing malignant from benign epithelial lesions of prostate. Prostate, 2007. 67(9): p. 907-10.
51. Tuxhorn, J. A., et al., Reactive stroma in human prostate cancer: induction of myofibroblast phenotype and extracellular matrix remodeling. Clin Cancer Res, 2002. 8(9): p. 2912-23.
52. Rowley, D. R., What might a stromal response mean to prostate cancer progression?Cancer Metastasis Rev, 1998. 17(4): p. 411-9.
53. Wang, Y., et al., Sex hormone-induced carcinogenesis in Rb-deficient prostate tissue. Cancer Res, 2000. 60(21): p. 6008-17.
54. Tuxhorn, J. A., G. E. Ayala, and D. R. Rowley, Reactive stroma in prostate cancer progression. J Urol, 2001. 166(6): p. 2472-83.
55. van der Heul-Nieuwenhuijsen, L., et al., Gene expression profiling of the human prostate zones. BJU Int, 2006. 98(4): p. 886-97.
56. Pflug, B. R., R. E. Reiter, and J. B. Nelson, Caveolin expression is decreased following androgen deprivation in human prostate cancer cell lines. Prostate, 1999. 40(4): p. 269-73.
57. Xin, W., et al., Dysregulation of the annexin family protein family is associated with prostate cancer progression. Am J Pathol, 2003. 162(1): p. 255-61.
58. Haywood-Reid, P. L., D. R. Zipf, and W. R. Springer, Quantification of integrin subunits on human prostatic cell lines—comparison of nontumorigenic and tumorigenic lines. Prostate, 1997. 31(1): p. 1-8.
59. Bae, I., et al., BRCA1 regulates gene expression for orderly mitotic progression. Cell Cycle, 2005. 4(11): p. 1641-66.
60. Sahadevan, K., et al., Selective over-expression of fibroblast growth factor receptors 1 and 4 in clinical prostate cancer. J Pathol, 2007. 213(1): p. 82-90.
61. Rhodes, D. R., et al., Meta-analysis of microarrays: interstudy validation of gene expression profiles reveals pathway dysregulation in prostate cancer. Cancer Res, 2002. 62(15): p. 4427-33.
62. Warnat, P., R. Eils, and B. Brors, Cross platformanalysis of cancer microarray data improves gene expression based classification of phenotypes. BMC Bioinformatics, 2005. 6: p. 265.
63. Yang, H. P., et al., Genetic variation in interleukin 8 and its receptor genes and its influence on the risk and prognosis of prostate cancer among Finnish men in a large cancer prevention trial. Eur J Cancer Prey, 2006. 15(3): p. 249-53.
64. DeConde, R. P., et al., Combining results of microarray experiments: a rank aggregation approach. Stat Appl Genet Mol Biol, 2006. 5: p. Articlel5.
65. Rodriguez-Canales, J., et al., Identification of a unique epigenetic sub-microenvironment in prostate cancer. J Pathol, 2007. 211(4): p. 410-9.
66. Ruifrok, A. C. and D. A. Johnston, Quantification of histochemical staining by color deconvolution. Anal Quant Cytol Histol, 2001. 23(4): p. 291-9.
67. Krajewska, M., Shinichi Kitada, Jane N. Winter, Daina Variakojis, Alan Lichtenstein, Dayong Zhai, Michael Cuddy, Xianshu Huang, Frederic Luciano, Cheryl H. Baker, Hoguen Kim6, Eunah Shin7, Susan Kennedy, Allen H. Olson, Andrzej Badzio, Jacek Jassem, Ivo Meinhold-Heerlein, Michael J. Duffy, Aaron D. Schimmer, Ming Tsao3, Ewan Brown, Anne Sawyers, Michael Andreeff1, Dan Mercola, Stan Krajewski and John C. Reed., Bcl-B Expression in Human Epithelial and Nonepithelial Malignancies Clinical Cancer Research, 2008. 14: p. 3011-3021.
68. Krajewska, M., et al., Analysis of apoptosis protein expression in early-stage colorectal cancer suggests opportunities for new prognostic biomarkers. Clin Cancer Res, 2005b 11(15): p. 5451-61.
69. Krajewska, M., et al., Tumor-associated alterations in caspase-14 expression in epithelial malignancies. Clin Cancer Res, 2005a. 11(15): p. 5462-71.
70. Turner, B. C., et al., BAG-1: a novel biomarker predicting long-term survival in early-stage breast cancer. J Clin Oncol, 2001. 19(4): p. 992-1000.
71. Krajewski, S., et al., Release of caspase-9 from mitochondria during neuronal apoptosis and cerebral ischemia. Proc Natl Acad Sci USA, 1999. 96(10): p. 5752-7.
72. Rabinovich, A., et al., Framework for parsing, visualizing and scoring tissue microarray images. IEEE Trans Inf Technol Biomed, 2006. 10(2): p. 209-19.
73. Krajewska, M., et al., Expression of BAG-1 protein correlates with aggressive behavior of prostate cancers. Prostate, 2006. 66(8): p. 801-10.
74. Meinhold-Heerlein, I., et al., Expression and potential role of Fas-associated phosphatase-1 in ovarian cancer. Am J Pathol, 2001. 158(4): p. 1335-44.
75. Ahlering, T. E. and D. W. Skarecky, Long-term outcome of detectable PSA levels after radical prostatectomy. Prostate Cancer Prostatic Dis, 2005. 8(2): p. 163-6.
76. Adley, B. P. and X. J. Yang, Application of alpha-methylacyl coenzyme A racemase immunohistochemistry in the diagnosis of prostate cancer: a review. Anal Quant Cytol Histol, 2006. 28(1): p. 1-13.
77. Hameed, O., J. Sublett, and P. A. Humphrey, Immunohistochemical stains for p63 and alpha-methylacyl-CoA racemase, versus a cocktail comprising both, in the diagnosis of prostatic carcinoma: a comparison of the immunohistochemical staining of 430 foci in radical prostatectomy and needle biopsy tissues. Am J Surg Pathol, 2005. 29(5): p. 579-87.
78. Herawi, M. and J. I. Epstein, Specialized stromal tumors of the prostate: a clinicopathologic study of 50 cases. Am J Surg Pathol, 2006. 30(6): p. 694-704.
79. Epstein, J. I. and M. Herawi, Prostate needle biopsies containing prostatic intraepithelial neoplasia or atypical foci suspicious for carcinoma: implications for patient care. J Urol, 2006. 175(3 Pt 1): p. 820-34.
80. Gonzalgo, M. L., et al., Relationship between primary Gleason pattern on needle biopsy and clinicopathologic outcomes among men with Gleason score 7 adenocarcinoma of the prostate. Urology, 2006. 67(1): p. 115-9.
81. Varma, M. and B. Jasani, Diagnostic utility of immunohistochemistry in morphologically difficult prostate cancer: review of current literature. Histopathology, 2005. 47(1): p. 1-16.
82. Rimm, D. L., et al., Tissue microarray: a new technology for amplification of tissue resources. Cancer J, 2001. 7(1): p. 24-31.
83. Camp, R. L., G. G. Chung, and D. L. Rimm, Automated subcellular localization and quantification of protein expression in tissue microarrays. Nat Med, 2002. 8(11): p. 1323-7.
84. Rubin, M. A., et al., Quantitative determination of expression of the prostate cancer protein alpha-methylacyl-CoA racemase using automated quantitative analysis (AQUA): a novel paradigm for automated and continuous biomarker measurements. Am J Pathol, 2004. 164(3): p. 831-40.
85. Prigozhina, N. L., et al., Plasma membrane assays and three-compartment image cytometry for high content screening. Assay Drug Dev Technol, 2007. 5(1): p. 29-48.
86. Mikic, I., et al., A live cell, image-based approach to understanding the enzymology and pharmacology of 2-bromopalmitate and palmitoylation. Methods Enzymol, 2006. 414: p. 150-87.

### Example 9

### Conversion of a Novel RNA-Based Prognostic Test for Prostate Cancer into a Clinical Assay

A. SPECIFIC AIMS. Nomograms are sets of clinical parameters that are used to estimate the risk of prostate cancer recurrence [1, 2]. We propose to improve on the current nomograms by including predictions based on gene expression.

We have used a novel strategy to identify and validate genes whose expression correlates with prostate cancer progression in either tumor tissue or in stroma near to tumor, across multiple independent microarray datasets. We will convert this set of expression differences into a clinical assay. Our proposed strategy involves monitoring a panel of RNAs, including some RNAs that predict the risk of disease recurrence, some RNAs for housekeeping genes (internal controls), and some RNAs that are used to determine the tissue composition of a prostate sample (tumor, stroma, BPH). The inclusion of RNAs to monitor tissue percentage allows only suitable prognostic markers to be monitored in each sample; those prognostic markers that are directed towards the primary tissue in that particular sample.

We will use an RNA detection strategy (QuantiGene Plex 2.0) that works on both fresh frozen and FFPE samples, and that can accurately monitor up to 36 different RNAs, simultaneously. The assay runs on the FDA-approved Luminex platform, already used in clinical labs. We will first screen our candidate RNAs for those that perform well on this platform using RNA from fresh frozen samples with known microarray expression patterns. Panels will then be applied to 150 tumor-enriched FFPE samples and 150 stroma-enriched (near to tumor), from prostate cancer patients, with up to two decades of clinical history. The best performing subset of genes will be assembled into two panels for clinical use, one for use in stroma-enriched samples, and the other to be used in tumor-enriched samples.

The long-term goal is to validate the classifiers in a prospective study on newly recruited prostatectomy samples.

B. Background and Significance.

Cancer and the Need for Prognostic Markers. Prostate cancer is the most common malignancy of males in the United States [3]. Patients newly diagnosed with advanced prostate cancer that do not yet have evidence of metastases are generally advised to submit to invasive therapies such as radical prostatectomy or radiation treatment. However, the majority of prostate cancers are a slow growing indolent form with a low risk of mortality. Patients with early stage disease and extremely favorable nomogram scores, suggesting indolence of the cancer, can instead opt for intensive vigilance. We propose the development of a gene-expression-based clinical test that makes a differential prognostic prediction between indolent and aggressive forms of prostate cancer. This test would provide an additional key aid to prostate cancer patients, and doctors, in making their treatment decisions, and will be particularly useful for those patients that are not at the extremes of the current nomogram scoring systems [1, 2].

While other studies to detect RNA-based prognosticators for prostate cancer have been performed, they have limited agreement with each other, and very limited overlap with prognosticators found by other methods [4-7]. We have developed a different method that identifies prognostic markers and we have cross-validated them across different data sets (detailed below). We now propose to convert a panel of these prognosticators into a useful clinical assay. We will use the QuantiGene Plex 2.0 Assay (Panomics, Inc., Fremont, Calif.), which is as sensitive as real time PCR but can be much more extensively multiplexed [8, 9]. The assay can detect up to 36 targets per well. The assay is based on the branched DNA (bDNA) technology, which amplifies signal directly from captured target RNA without purification or reverse transcription. RNA quantitation is performed directly from fresh frozen tissue or from formalin-fixed, paraffin-embedded (FFPE) tissue homogenates, and is relatively insensitive to RNA degradation and to chemical modifications introduced by formalin-fixation [10, 11]. The method is already in the FDA-approved clinical diagnostic VERSANT 3.0 assays for HIV, HCV and HBV viral load [12] and has been used in biomarker discovery, secondary screening, microarray validation, quantification of RNAi knockdowns and predictive toxicology [11, 13-15].

C. PRELIMINARY STUDIES. The key to this project is the set of genes that we will put into the prognostic assay. We describe how we obtained these genes in some detail here.

We previously developed methods to determine the genes preferentially expressed by the three major cell types of tumor-bearing prostate tissue: tumor epithelial cells, benign epithelial cells (BPH) and stromal cells [16]. We have now extended this method so that we can now identify transcription changes that correlate with early cancer recurrence in one or more of these three cell types. In addition to transcription changes in tumor cells that correlate with recurrence, we find that prognostic changes also occur in stroma near to tumor but not in BPH. We have validated a subset of these new recurrence-related genes using independent publicly available microarray data sets. Table 31 summarizes the data sets we have analyzed from various sources, including our own prostatectomy samples.

TABLE 31
Prostate cancer expression microarray data sets

| | Array | | | Non- | |
|---|---|---|---|---|---|
| Data Sets | platform | Targets | Recurrent | Recurrent | Reference |
| 1 | U133Plus2 | 54,675 | 27 | 38 | Our |
| | | | | | unpublished |
| | | | | | data |
| 2 | U133A | 22,283 | 30 | 26 | Our |
| | | | | | unpublished |
| | | | | | data |
| 3 | Illumina | 511 | 18 | 63 | [4] |
| 4 | U133A | 22,283 | 29 | 42 | [7] |
| 5 | U95Av2 | 12,626 | 8 | 13 | [6] |
| 6 | U95Av2 | 12,626 | 9 | 14 | [5] |

Identification of cell-specific genes. Most previous experiments to determine expression profiles of solid tumors using microarrays involved “enriched” tumor fractions. There are three limitations of this strategy. First, samples vary in purity, introducing an error due to various amounts of accompanying tissue types. Second, the change in gene expression of other cell types is subsumed in a single number, obscuring the unique profiles of these accompanying cell types. Third, substantial amounts of stroma are intrinsic to the structure of nearly all prostate tumors. We devised a method for the deconvolution of average cell-specific gene expression from a set of samples containing different mixtures of cell types [16]. Estimates of the amount of three major cell types were made: tumor epithelial cells (tumor, T), epithelium of benign prostatic hyperplasia (BPH, B), and stromal cells (S, including pooled smooth muscle, connective tissue, infiltrating immune cells, and vascular elements). The amount of mRNA (Affymetrix signal intensity, GO from a given gene is the sum of the amount of each cell type multiplied by the intrinsic expression, A, of that gene by the given cell type:

Gᵢⱼ=β_{BPH,j}x_{BPH,i}+β_{T,j}x_{T,i}β_{S,j}x_{S,i}εᵢⱼ (1)

where Xᵢ is the proportion of each cell type and ε is the error. The model identified hundreds of genes significantly more expressed in only one tissue and examples were validated by laser capture micro-dissection and immunohistochemistry [16].

In silico estimates of tissue percentages. Estimates of tissue percentages made by pathologists for all the samples in data set 1, 2 and 3 allowed identification of individual transcript levels that correlated best with tissue percentage. The expression levels of each of these overlapping genes were fitted to a simple linear model for each tissue type and were ranked by their correlation coefficient. A subset of the top genes from one data set was subsequently used to predict tissue percentage in the other data set. The Pearson correlation coefficients between predicted cell type percentage (tumor, stroma and BPH cells) and pathologist's estimates for all pairwise predictions of the three data sets range from 0.45-0.87 (p<0.001 in all comparisons).

Estimation of cell type percentage proved to be highly relevant. In data set 4, recurrent cases had a systematically higher percentage of tumor tissue than non-recurrent cases. Unless recognized and taken into account, this skew would generate false expression-derived estimates regarding recurrence.

Identification of cell-specific biomarkers of aggressive prostate cancer. We have now extended equation 1 to identify genes specific to cell-type and aggression, for cases with known follow-up history. To obtain cell-specific gene expression for both recurrent and non-recurrent cases, the summation of equation 1 is simply segregated to reserve terms with A coefficients for non-recurrent cases and denoting recurrent cases (rs) at the end with a separate coefficient, γ

Gᵢⱼ=β_{BPH,j}x_{BPH,i}+β_{T,j}x_{T,i}+β_{S,j}x_{S,i})+rs(γ_{BPH,j}x_{BPH,i}+γ_{T,j}x_{T,i}+γ_{S,j}x_{S,i})+εᵢⱼ (2)

Multiple linear regression (MLR) analysis was carried out leading to the calculation of all βⱼ, all γⱼ, and their associated t-statistic values. Thus, estimates of the intrinsic expression of three cell types (T, S and BPH) for non-recurrent and recurrent prostate cancer were derived.

In data set 1 (U133Plus2.0 array), for example, 928 differentially regulated genes were identified in early recurrent cancer types at an adjusted p value of less than 0.05, including 405 tumor- and 561 stroma-related prognostic genes. In both data sets 1 and 2, the most significant changes were observed in the stromal tissue portion of specimens that were from near tumor (reactive stroma). The ability to look for changes in expression in stroma during recurrence is one of the major advantages of our approach.

Confirmation of Prognostic Genes using Independent Data Sets (Cross-Validation). The six available expression microarray data sets with information on prostate cancer recurrence (Table 31) allowed identification of that subset of candidate prognosticators that could be validated. We filtered all sets for y with p<0.05; then mapped identical Affymetrix probes (data set 1, 2, 4, 5 and 6) or gene symbol (data set 2). Finally, we identified genes that occurred in both compared data sets, and showed the same direction of change in differential expression between recurrent and non-recurring samples. Overall, 152 of 185 (82.2%) genes were concordant across pairs of data sets (p<10⁻¹⁸). About one third of the 152 concordant genes correspond to those previously reported by others as related to outcome in prostate cancer. About a quarter may be in error (false discovery rate given that 31 of 185 were not concordant). Some sets of genes are functionally related to biological processes considered important in the progression of prostate cancer, exemplified by several members of the Wnt signal transduction pathway.

The enormous tissue percentage diversity among published data sets (all “tumor enriched” sets had some samples with less than 30% tumor, according to our in silico analysis) and a frequent bias in tumor percentages between recurrent and non-recurrent cases (leading to any tumor-specific gene being erroneously associated with recurrence) provides two explanations for the previous struggle of the community to find a valid recurrence-specific signature in any one data set.

Gene Expression Quantification Using the QuantiGene Plex 2.0 assay. We have tested the sensitivity and the technical and biological accuracy of the assay using a panel of genes in a 10-Plex. The ten-gene panel included two housekeeping genes and eight genes with cell type percentage predictive power for prostate tumor, stroma, and BPH. The assay was performed on 12 fresh frozen prostate cancer samples and 9 FPEE samples with various amounts of tumor, stroma, and BPH.

A standard curve for the housekeeping gene ribosomal protein S20 proved that the Plex 2.0 assay is highly reproducible and sensitive with a wide dynamic range (not shown).

Transcripts for all ten genes were accurately measured over a wide dynamic range when the template amount was over 33 ng. The gene expression levels for all eight tissue-specific genes detected by either the Plex 2.0 assay, or the Affymetrix U133P2 array using the same RNA samples, had correlation coefficients ranging from 0.64 to 0.89. Moreover, all eight tissue-enriched genes showed good correlations with their respective cell type percentages in FFPE samples. These preliminary experiments demonstrate that the Plex 2.0 assay is a very sensitive and reproducible method, consistent with microarray data.

D. RESEARCH DESIGN AND METHODS. The thousands of tissue specific genes and over 150 candidate prognostic genes that we have identified will vary in their practical usefulness. Furthermore, not all of these genes will translate to a particular assay platform, due to circumstances such as splicing variants that may not behave identically. This project will find a subset of high performance genes for our chosen assay strategy, gleaned from among the many high-confidence candidate genes we have identified.

We will convert the gene markers into an assay that can be easily adapted in a clinical lab, using the Plex 2.0 assay on FFPE samples (no RNA extraction or reverse transcription required). For probe validation, assays will be performed on 24 total RNA samples which already have previously reported microarray data. Probes that correlate best with the microarray data will be used to analyze 150 FFPE samples with annotated recurrence status (over a decade of post-surgery follow-up in most cases). A classifier that can distinguish indolent and/or aggressive cases will be developed and outcome prediction accuracy will be estimated by cross-validation.

Step 1. Select Candidate Genes for Further Validation. We have selected a list of gene biomarkers for further analysis, including 75 prognostic marker genes from our studies and 25 that are found in at least one of our datasets and in the literature, 30 tissue component prediction genes, and 4 housekeeping genes which represent relatively low, medium and high expression levels.

Step 2. QuantiGene Plex Assay Probe Design and Validation.

Frozen Tissue Samples. 24 total RNA samples that already have Affymetrix gene expression data will be used in the Plex 2.0 assay. The RNA samples will be selected to encompass a wide range of tissue percentages and equal numbers of non-recurrent and recurrent cases. Probes of the Plex 2.0 assay will be designed by Panomics. Each panel of the Plex 2.0 assay will contain up to 36 genes. We will test four panels, totaling 130 or more candidate genes. The assay will be performed using our Bio-Plex system which relies on FACS sorting of fluorescently encoded beads.

Selection of Genes for Future Use. Genes that show significant correlation between the Plex assay and Affymetrix assay will be kept for further analysis. Genes with very low signal or low variance in these assays will be eliminated from further analysis. We will combine the top performing genes into three panels (36 genes per panel) for further study. If necessary, more potentially useful prognostic or tissue-enriched transcripts will be screened.

Step 3. Develop Classifiers for Recurrence Prediction. FFPE Samples. We will acquire a set of 150 archived prostate cancer samples from the SPECS study for validation. Two samples will be selected from each block. One will be tumor-enriched (>70% tumor cells) and the other stroma-enriched (>70% stroma cells near to tumor: “Reactive stroma”) as estimated by pathologists. These blocks have 8-20 years of associated clinical data and represent a range of overall survival and time to recurrence. Gleason scores range from 5˜8. Samples will be coded for blind analysis. Plex 2.0 Assays will be performed on the three panels of above selected genes.

Outcome Prediction. We will first use a subset of the samples with the pathologists' estimates of cell type percentages to develop linear models of cell type component prediction. Cell type percentages of the remaining samples will be estimated using these linear models and the most predictive markers will be identified to be retained in the ultimate clinical assay.

Samples will be divided into tumor-enriched samples, stroma-enriched samples. Those samples that prove not to be suitably enriched will be set aside. We will use the appropriate tissue-enriched samples to develop classifiers that distinguish aggressive and indolent cancers using Prediction Analysis for Microarrays (PAM) [17] and Support Vector Machine (SVM) [18, 19] approaches. Misclassification error will be estimated by the 10-fold cross-validation or the leave one out strategy. These tools will be implemented in R (http://www.r-projectorg/). Two classifiers will be developed, one for tumor-enriched samples and one for stroma-enriched samples.

We will also attempt in silico correction of transcript levels based on the tissue percentage markers present in each multiplex. We will attempt to adjust signals to reflect the tissue percentages by simple linear regression and determine if this variable improves disease outcome prediction.

Pre- and post operation PSA, pathology T stage, and Gleason scores are available for all cases. Thus, using these parameters plus our RNA-based classifier, the nomogram-predicted disease free survival can be calculated.

Final predictive set. The initial four panels of up to 36 genes, each, will be reduced to three panels after initial screening. Then these three panels used in the FFPE study will be further condensed into just two panels that contain only useful genes for tissue percentage estimation and for prognosis: one panel for stroma-enriched samples and one for tumor-enriched samples. Both panels will measure up to 10 RNAs for estimating tissue percentage, 25 RNAs for prognosis, and 3 or more housekeeping controls.

Further Studies.

Application to Biopsies. We have found biopsies to be an excellent source of RNA. If any stroma biomarkers are associated with recurrence, we will test the Plex 2.0 assay on 10 of our hundreds of snap frozen biopsy samples to determine technical feasibility. It is possible that biopsies that are negative for cancer may still have regions that are close enough to the missed tumor that they show “reactive” gene changes. This would revolutionize the assessment of patients that are negative for cancer upon biopsy.

More Sophisticated Class Prediction Algorithms. In this project, we propose to use in silico cell type composition prediction to estimate tumor percentages only for sample quality control. However, knowledge of tissue composition opens up opportunities for many intellectual advances in data analysis. We are developing a new classification method which takes advantage of cell composition information without rejecting any high quality data, and results in better performance than PAM and SVM-based predictions [20].

Signaling Pathway Analysis for Understanding Prostate Cancer Progression. Our preliminary study on pathway analysis shows that our newly identified predictive markers for recurrence are significantly enriched for elements involved in cancer related pathways, exemplified by the Wnt signaling pathway. One of our long term goals is to explore the mechanisms of cancer-related pathways that are cross-validated in multiple data sets using tools such as DAVID (The Database for Annotation, Visualization and Integrated Discovery) [21, 22]. These pathways are potential targets for novel therapeutic treatment.

1. Unique in silico tissue composition prediction strategy based on gene expression profiling. Large variations in the proportion of tissue components in prostate cancer tissue samples lead to considerable noise and even misleading results in mining microarrays data for prognosticators. We have generated and validated linear models for tissue component estimations based on gene expression levels. Lists of 10˜20 genes that define tumor, stroma and BPH tissue, allow the proportion of each of these tissues to be determined from gene expression profiles, alone. This novel approach of in silico tissue component prediction will be used for quality control by determining the major cell components in each clinical RNA sample.

2. Unique prognostic gene biomarkers. Using a multiple linear regression model which integrates tissue component percentages, we have identified a list of tumor- and reactive stroma-associated prognostic biomarkers, which can distinguish indolent and aggressive prostate cancer. Markers were then cross-validated between different microarray data sets produced by different research groups. Most of these prognostic markers were not previously identified by other studies. This is a simple and yet novel approach to find better, more precise, prognosticators for disease progression.

3. Accurate and sensitive multiple gene expression quantitation. A single prostate cancer prognostic marker is unlikely to be able to classify patients. Instead, a group of markers will be needed to account for the genetic variability of patients and the variability in cancer progression. The QuantiGene Plex 2.0 assay (Panomics, Inc) allows simultaneous quantification of multiple RNA targets directly from tissue homogenates. The assay does not require RNA purification, reverse transcription, or target amplification, because it combines branched DNA (bDNA) signal amplification technology and xMAP® (multi-analyte profiling) beads. The assay uses the FDA approved Luminex system already found in clinical labs.

Our data prove the accuracy and sensitivity of the assay, and the ability to predict tissue proportions in FFPE samples. We will convert a large number of previously identified and successfully cross-validated prognostic genes into the QuantiGene assay system that can then be easily adopted by clinical labs. The QuantiGene assay gene panel will be tested on our large collection of FFPE samples that have up to decades of patient data after surgery.

### REFERENCES

1. Han, W. D., et al., Up-regulation of LRP16 mRNA by 17beta-estradiol through activation of estrogen receptor alpha (ERalpha), but not ERbeta, and promotion of human breast cancer MCF-7 cell proliferation: a preliminary report. Endocr Relat Cancer, 2003. 10(2): p. 217-24.
2. Kattan, M. W., T. M. Wheeler, and P. T. Scardino, Postoperative nomogram for disease recurrence after radical prostatectomy for prostate cancer. J Clin Oncol, 1999. 17(5): p. 1499-507.
3. Reis, L., Eisner, M., Kosary, C., Hankey, B., Miller, B., Clegg, L., Edwards, B., SEER Cancer Statistics Review, 1973-1999. book, National Institutes of Health, Betheda, Md., 2002 (2002).
4. Bibikova, M., et al., Expression signatures that correlated with Gleason score and relapse in prostate cancer. Genomics, 2007. 89(6): p. 666-72.
5. LaTulippe, E., et al., Comprehensive gene expression analysis of prostate cancer reveals distinct transcriptional programs associated with metastatic disease. Cancer Res, 2002. 62(15): p. 4499-506.
6. Singh, D., et al., Gene expression correlates of clinical prostate cancer behavior. Cancer Cell, 2002. 1(2): p. 203-9.
7. Stephenson, A. J., et al., Integration of gene expression profiling and clinical variables to predict prostate carcinoma recurrence after radical prostatectomy. Cancer, 2005. 104(2): p. 290-8.
8. Arikawa, E., et al., Cross-platform comparison of SYBR Green real-time PCR with TaqMan PCR, microarrays and other gene expression measurement technologies evaluated in the MicroArray Quality Control (MAQC) study. BMC Genomics, 2008. 9: p. 328.
9. Canales, R. D., et al., Evaluation of DNA microarray results with quantitative gene expression platforms. Nat Biotechnol, 2006. 24(9): p. 1115-22.
10. Beer, D. G., et al., Gene-expression profiles predict survival of patients with lung adenocarcinoma. Nat Med, 2002. 8(8): p. 816-24.
11. Knudsen, B. S., et al., Evaluation of the branched-chain DNA assay for measurement of RNA in formalin-fixed tissues. J Mol Diagn, 2008. 10(2): p. 169-76.
12. Elbeik, T., et al., Multicenter evaluation of the performance characteristics of the bayer VERSANT HCV RNA 3.0 assay (bDNA). J Clin Microbiol, 2004. 42(2): p. 563-9.
13. Calcagno, A. M., et al., Single-step doxorubicin-selected cancer cells overexpress the ABCG2 drug transporter through epigenetic changes. Br J Cancer, 2008. 98(9): p. 1515-24.
14. John, M., et al., Effective RNAi-mediated gene silencing without interruption of the endogenous microRNA pathway. Nature, 2007. 449(7163): p. 745-7.
15. Yang, W., et al., Direct quantification of gene expression in homogenates of formalin-fixed, paraffin-embedded tissues. Biotechniques, 2006. 40(4): p. 481-6.
16. Stuart, R. O., Wachsman William, Berry Charles C., Arden Karen, Goodison Steven, Klacansky Igor, McClelland Michael, Wang-Rodriquez Jessica, Wasserman Linda, Sawyers, Ann, Yipeng, Wang, Kalcheva, Iveata, Tarin David, Mercola Dan., In silico dissection of cell-type associated patterns of gene expression in prostate cancer. Proceeding of the National Academy of Sciences U.S.A., 2004. 101: p. 615-620.
17. Tibshirani, R., et al., Diagnosis of multiple cancer types by shrunken centroids of gene expression. Proc Natl Acad Sci USA, 2002. 99(10): p. 6567-72.
18. Ramaswamy, S., et al., Multiclass cancer diagnosis using tumor gene expression signatures. Proc Natl Acad Sci USA, 2001. 98(26): p. 15149-54.
19. Su, A. I., et al., Molecular classification of human carcinomas by use of gene expression signatures. Cancer Res, 2001. 61(20): p. 7388-93.
20. Wang, Y., et al., A New Bi-Model Classifier for Predicting Outcomes of Prostate Cancer Patients. JSM Proceedings, 2008.
21. Dennis, G., Jr., et al., DAVID: Database for Annotation, Visualization, and Integrated Discovery. Genome Biol, 2003. 4(5): p. P3.
22. Huang da, W., et al., DAVID Bioinformatics Resources: expanded annotation database and novel algorithms to better extract biology from large gene lists. Nucleic Acids Res, 2007. 35(Web Server issue): p. W169-75.

### Example 10

### Increasing Sample Size does not Boost Power If Confounding Factors are not Controlled—A Study of Prostate Cancer with Microarray

### Analysis of Prostate Cancer Data

We recently published a dataset for prostate cancer study (publicly available at GEO database with access number GSE8218) [3]. This dataset consists of 136 samples from 82 patients who went through prostatectomy. Of these 82 patients, 45 underwent disease relapse, 33 did not and the remaining 4 were unknown. Here we used the 130 samples with definitive relapse status for this study. In some cases, more than one sample was collected from different regions of prostate of the same patient, for example, from tumor-enriched microdissected tissue and from nontumor tissue from 1.5 cm from tumor (usually the contralateral lobe). For each sample which was used for microarray assay, four pathologists independently reviewed the hematoxylin and eosin (H&E) stained sections and estimated the percentages of three major cell components, i.e., tumor, stroma and BPH. The goal of this study is to identify genes that are associated with disease progression in tumor cells or maybe in other types of cells which indicate gene expression changes in the tumor micro-environment [16].

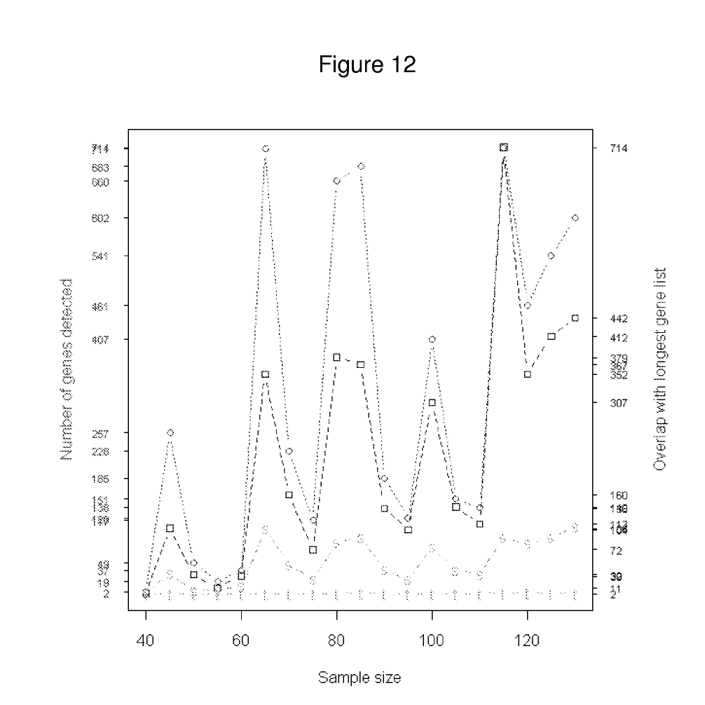

At first, we did differential analysis on all the 130 samples using the LIMMA package (http://www.bioconductor.org) in R [5]. We identified 602 altered genes between relapse and non-relapse groups by the criterion of B>0, where B represents log-likelihood-ratio of being differentially expressed versus being equivalently expressed. Thus, B>0 indicates that the gene under consideration has altered expression between relapse and non-relapse groups. The same criterion applied to the gene selection in the subsequent analyses. We then randomly selected a subset of 40, 45, . . . , 120, 125 samples from the data and carried out differential expression analysis respectively. If increase of sample size boosts power, we expect to see that more genes are detected when sample size becomes larger and the overlap of the signatures detected at different sample sizes is large, i.e., the circles and squares in FIG. 12 are supposed to stay close to each other and go upward steadily. Nevertheless, as shown in FIG. 12, the number of detected genes fluctuated as sample size increased with maximum detection (666 genes) when 120 randomly selected samples were used (circles). We compared different gene lists identified to the longest gene list of 666 genes in FIG. 12 (squares) which showed only moderate overlap.

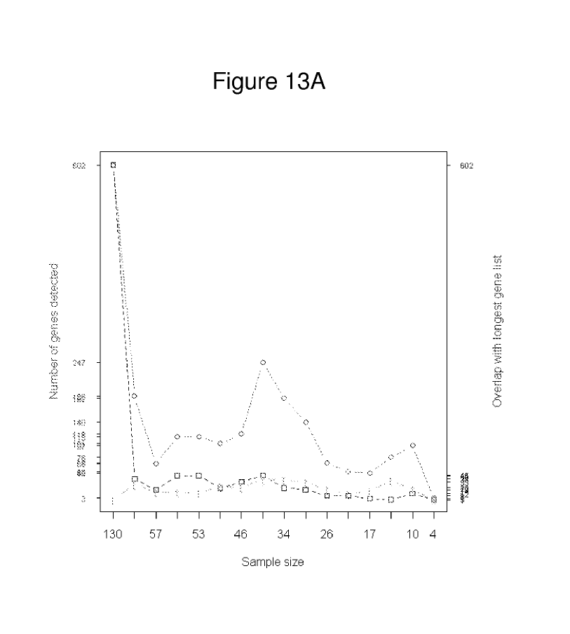

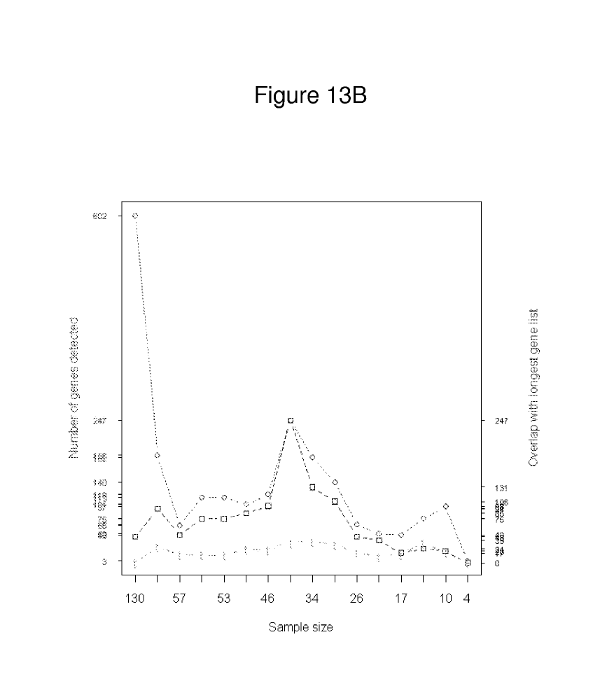

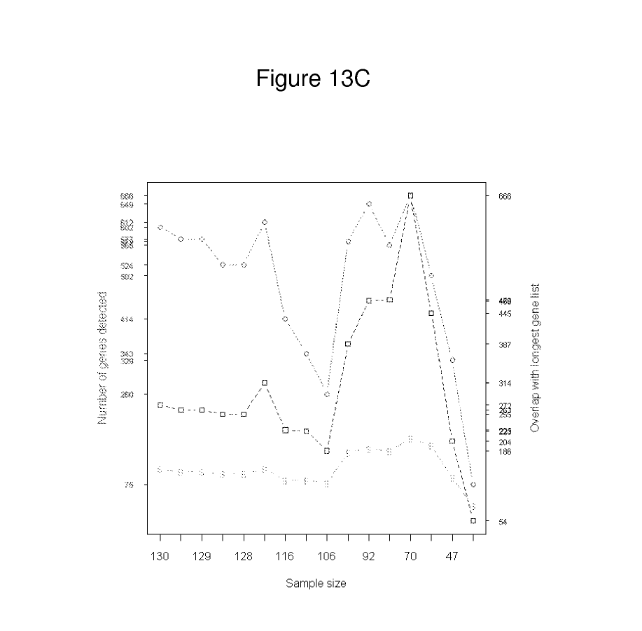

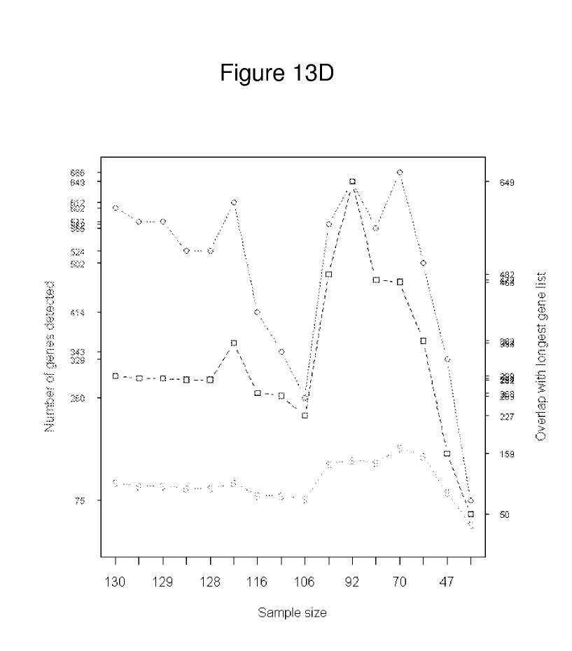

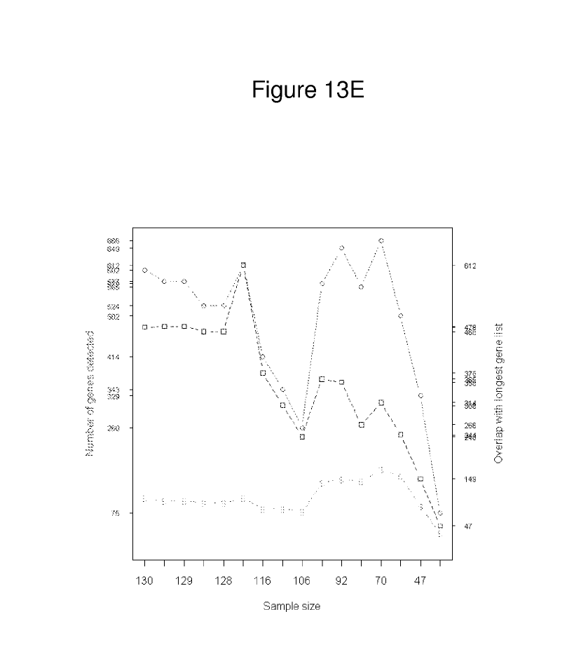

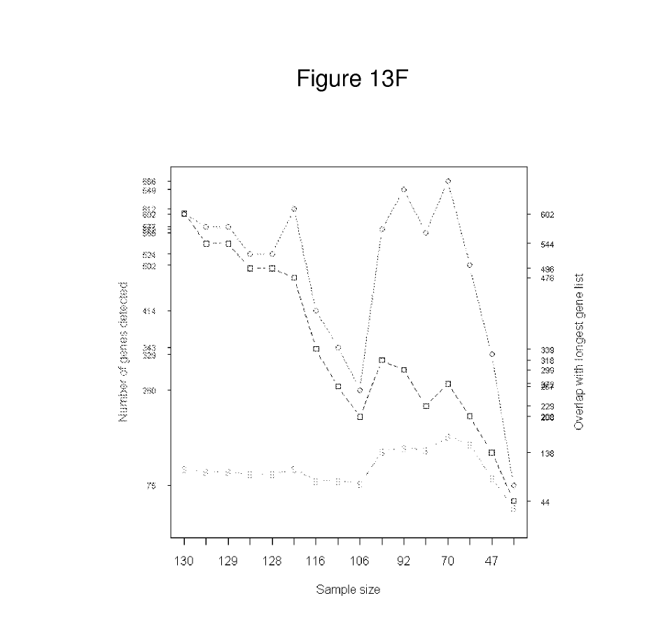

Next, we selected samples by stepwise enriching the tumor or stroma components which are two major types of cells in prostate tissue. Specifically, we used T, k % (k=0, 5, . . . , 70, 75) as cutoff for sample selection, where T stands for the percentage for tumor component. The number of genes identified in each case were summarized in FIG. 13A. The maximum detection (602 genes) occurred when all 130 samples were included in the analysis. However, the overlap between these 602 genes with the gene lists detected at other points were very low (the squares were very much separated from the circles). In particular, the overlap between these 602 genes with the gene lists detected for tumor enriched samples in the right half of the plot was very low, indicating that many of the 602 genes were false discoveries due to the diversity in terms of cell composition of samples. This suggested that employing all the 130 samples available is not the optimal strategy. However, there was another peak for the curve indicated by the circles when 40 samples (with tumor component greater than 35%) were used. The overlap between the detected genes at this point (as new reference gene list) with other gene lists near this point (sample size 22 to 49) was plotted in FIG. 13B. The overlaps were high (80%, curves indicated by circles and squares stuck together within this region), suggesting consistent discoveries among these assays (FIG. 13B). We observed that at the right end of the plot the number of detected genes rises at sample size=17 and less but the overlap with the list of 247 genes (identified at sample size=40; Table 33) kept dropping. This odd behavior was ascribed to the tiny sample size, for example, only 4 to 17 samples were included, which diminished power but enlarged chance of incurring false positives.

A similar phenomenon was observed when we investigate relapse-associated stromal genes. There were two peaks for the genes predicted to associated with recurrence (circles) at sample size 70 and 92 in the right half of the plot (stroma enriched samples). The overlap between the genes identified at these two points and gene lists around these two points (24 to 106) were fairly high (≧76%, see FIGS. 13C and 13D). In the left half of the plot, the detection rates were also high when most samples were included (sample size=128 in FIG. 13E; sample size=130 in FIG. 13F). However, the overlap between the detected genes at those points and gene lists identified at right end of the plot is very low, indicating that many detected genes were false positives if most samples were included. Note that the sample size at the right end of these plots is still reasonably large (34 to 60) compared to that of plots for genes putatively from tumor; therefore, we did not see the bending up of the curve indicated by the circles that occurs in FIGS. 13A-13B which indicated increased false positives. However, owing to the reduced power caused by fewer samples, many interested genes were missed (low detection rate at the right end of the plots compared to the detection rates when sample size=70 to 92).

The original paper dealt with the heterogeneous samples via using a multiple-linear-regression (MLR) model by which the observed Affymetrix gene expression values are described as linear combination of the contribution from different types of cells [3] [17]. Specifically, the following model was applied to the expression data for each gene,

$$g = {b}_{0} + {\sum }_{j = 1}^{C} {b}_{j} {p}_{j} + I ( RS = 1 ) \times {\sum }_{j = 1}^{C} {γ}_{j} {p}_{j} + ɛ , \qquad ( 1 )$$

where g is the observed expression for a gene, b₀ is the grand mean, C=3 indicating 3 types of cell component, p, is the percentage of cell type j, bⱼ represent the expression of this gene in cell type j when the case is non-relapse, γⱼ is the extra expression (either up- or down-regulated) in cell type j when the case relapses, and finally I(RS=1) is an indicator variable with I=1 if the case relapses (denoted by RS=1) and I=0 if the case does not recur (denoted by RS=0). We reanalyzed the data with exactly the same method and detected 119 relapse-associated genes in tumor and 247 relapse-associated gene in stroma. These two gene lists have 36 and 169 genes in common respectively with the 247 genes identified for tumor (sample size=40 in FIG. 13B) and 666 genes identified in stroma (sample size=70 in FIG. 13C) by t-test. We considered that the MLR analysis was more desirable than t-test (e.g., LIMMA) because (1) using the percentage data as covariates for regression analysis is more accurate than selecting samples based on the percentage cutoff, and (2) all samples are effectively used for calculation leading to increased power. However, precise percentage estimation data are not commonly available for many studies; in most cases, samples were only roughly classified into either tumor-enriched or stroma-enriched categories. Therefore, t-test still applies prevalently. To compare the results from these two analyses (t-test based on enriched samples and MLR), we added green/gold curve to each plot of FIG. 12 and FIG. 13 denoting the overlap between each identified gene lists by t-test and tumor/stroma genes identified with MLR. Here we assume that cell-type specific genes identified with MLR are more reliable based on above reasoning; thus, we try to validate results of t-test by MLR results. For random experiment (FIG. 12), the overlaps were limited and did not demonstrate any visible pattern as sample size increased. However, for stepwise enrichment experiment (FIG. 13), the overlaps were much improved and showed bell-shaped pattern as expected (with maximum at peaks of blue curves FIG. 13B-13D). We presume that these 247 tumor genes and 666 stroma genes identified by t-test were most close to reality because the optimal subset of samples were used by balancing sample size and homogeneity between samples. We also calculated the empirical p-values for the overlap between tumor/stroma gene lists identified with these two approaches as follows.

Suppose we calculate significance level for overlap of two tumor gene lists, i.e., 119 genes by MLR and 247 genes by t-test. Let count=0. From ˜22,000 genes, we randomly selected two gene lists of length 119 and 247, respectively. Not that 119 and 247 are the lengths of genes identified separately by t-test and MLR. If the overlap of the two randomly selected gene lists is equal or greater than 36 (observed overlap between these two tumor gene lists), we let count increase by 1. We repeated this process 10,000 times and the p-value of the observed overlap of tumor genes is calculated as

p=count/10000.

By the same means, we calculated the significance level for overlap of two stroma gene lists as well. Both p-values for tumor overlapping genes and stroma overlapping genes were ≦0.0001. This again verified the discoveries by t-test with stepwise enriched samples.

Simulated Study

In this section, we generated a dataset consisting of 200 samples each of which is composed of three types of cells. This is to mimic the situation we are facing for prostate cancer study. We randomly assigned the 200 samples into either case group (denoted by 1) or control group (denoted by 0). Here case means aggressive prostate cancers which will progress even after surgical removal prostate gland; while control denotes indolent prostate cancer which will not recur after prostatectomy. For each sample, the percentages of three cell types were simulated as follows. We let cell type 3 (BPH) be the minority cell which takes up to 10% volume in tissues; thus, we first generated the percentage of cell type 3 (×3) from uniform distribution U(0, 0.1). We then generated the percentage of cell type 1 (×1 for tumor) from U(0,1−×3), and the percentage of cell type 2 (×2 for stroma) is therefore 1−×1−×3. For each sample, we simulated expression data for 1000 gene as follows. We let gene 1 to 60 have altered expression in cell type 1 between case and control. The differences in terms of expression for gene 1 to 20, gene 21 to 40 and gene 41 to 60 are set to 0.5, 1.0 and 2.0, respectively. The same setting was used for generating differentially expressed genes for cell type 2 (gene 61 to 120). Due to the small load for cell type 3, we assume that the difference in cell type 3 between case and control is undetectable, so we did not simulate differentially expressed genes for cell type 3.

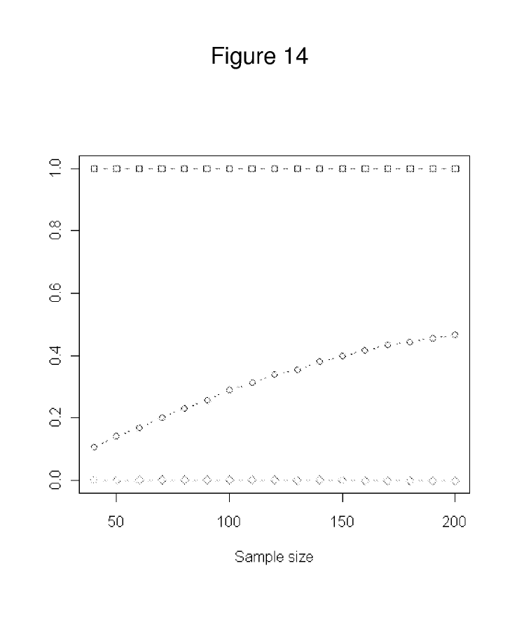

First, we randomly selected a subset of 40, 50, . . . , 190, 200 samples from the data and carried out differential expression analysis using LIMMA. The sensitivity, specificity and false discovery rate had been logged in each situation. Such analysis was repeated 100 times and the average operating characteristic is summarized in FIG. 14. The sensitivity or power went up as sample size increased, however, the detection rate was limited (maximum 46.7%). Note that the specificity and false discovery rate were steadily satisfactory (very close to 0).

Considering the heterogeneity in cell composition, we then selected samples by stepwise enriching one type of cell. Specifically, we included samples with ×1, k % (k=0, 5, . . . , 85, 90) in expression comparison procedure, and then identified genes that are differentially expressed in cell type 1 between case and control. With varying cutoff, the number of samples included in analysis and the sensitivity or power achieved by these samples are summarized in Table 32. Obviously, the maximum sensitivity or power is 73.3% which is much higher than any figures attained by randomly selected sample in FIG. 14. In addition, the maximum sensitivity or power achieved when ×1, 65%, neither too small nor too large in terms of the content of cell type 1 (or the number of samples included in the calculation). If the selected cutoff is too small, most samples will be included. This is like what we observed in previous assay when sample size is close to upper limit (see FIG. 14). In this case, the variation caused by mixed tissue is likely to impair detection power. However, if the selected cutoff is too large, too few samples will be included in the analysis, leading to a reduced power. For example, if we use ×1, 90% for sample selection, only 9 samples (5 controls and 4 cases) were selected. The sensitivity or power in this situation is only 43%. This is very similar to the observation in prostate cancer data analysis which showed a bending-down detection curve when sample size is near 0 (FIG. 13A-13B). There is a trade off between size and level of homogeneity of samples. Both factors positively contribute to power but never benefit from each other as if type I and type II errors in statistical hypothesis test. This lesson tells us that carefully selecting samples from resource is superior to utilizing all available samples indiscriminately.

Finally, we applied MLR to the simulated data and the results were much improved compared to the regular t-test with enriched samples (Table 32). This is what we expected and attested plausibility of validating results of t-test by using results of MLR analysis.

TABLE 32
Operating characteristics for MLR analysis.

| | Sensitivity | Specificity |
|---|---|---|
| Tumor genes | 91.7% | 96.0% |
| Stroma genes | 96.7% | 96.0% |

### REFERENCES

1. Blalock, E. M., Geddes, J. W., Chen, K. C., Porter, N. M., Markesbery, W. R., Landfield, P. W.: Incipient alzheimer's disease: Microarray correlation analyses reveal major transcriptional and tumor suppressor responses. Proceedings of the National Academy of Sciences of the United States of America 101 (2004) 2173-2178
2. Schena, M., Shalon, D., Davis, R. W., Brown, P. O.: Quantitative monitoring of gene-expression patterns with a complementary-dna microarray. Science 270(5235) (1995) 467-470
3. Stuart, R. O., Wachsman, W., Berry, C. C., Wang-Rodriguez, J., Wasserman, L., Klacansky, I., Masys, D., Arden, K., Goodison, S., McClelland, M., Wang, Y. P., Sawyers, A., Kalcheva, I., Tarin, D., Mercola, D.: In silico dissection of cell-type-associated patterns of gene expression in prostate cancer. Proceedings of the National Academy of Sciences of the United States of America 101(2) (2004) 615-620
4. Koziol, J. A., Feng, A. C., Jia, Z. Y., Wang, Y. P., Goodison, S., McClelland, M., Mercola, D.: The wisdom of the commons: ensemble tree classifiers for prostate cancer prognosis. Bioinformatics 25(1) (2009) 54-60
5. Smyth, G. K.: Linear models and empirical bayes methods for assessing differential expression in microarray experiments. Statistical Applications in Genetics and Molecular Biology 3 (2004) Article 3
6. Tusher, V. G., Tibshirani, R., Chu, G.: Significance analysis of microarrays applied to the ionizing radiation response. Proceedings of the National Academy of Sciences of the United States of America 98 (2001) 5116-5121
7. Jia, Z., Xu, S.: Bayesian mixture model analysis for detecting differentially expressed genes. International Journal of Plant Genomics 2008 (2008) Article ID 892927, 12 pages
8. Fan, C., Oh, D. S., Wessels, L., Weigelt, B., Nuyten, D. S. A., Nobel, A. B., van't Veer, L. J., Perou, C. M.: Concordance among gene-expression-based predictors for breast cancer. New England Journal of Medicine 355(6) (2006) 560-569
9. Chang, H. Y., Sneddon, J. B., Alizadeh, A. A., Sood, R., West, R. B., Montgomery, K., Chi, J. T., van de Rijn, M., Botstein, D., Brown, P. O.: Gene expression signature of fibroblast serum response predicts human cancer progression: Similarities between tumors and wounds. Plos Biology 2(2) (2004) 206-214
10. Paik, S., Shak, S., Tang, G., Kim, C., Baker, J., Cronin, M., Baehner, F. L., Walker, M. G., Watson, D., Park, T., Hiller, W., Fisher, E. R., Wickerham, D. L., Bryant, J., Wolmark, N.: A multigene assay to predict recurrence of tamoxifen-treated, node-negative breast cancer. New England Journal of Medicine 351(27) (2004) 2817-2826
11. Sorlie, T., Perou, C. M., Tibshirani, R., Aas, T., Geisler, S., Johnsen, H., Hastie, T., Eisen, M. B., van de Rijn, M., Jeffrey, S. S., Thorsen, T., Quist, H., Matese, J. C., Brown, P. O., Botstein, D., Lonning, P. E., Borresen-Dale, A. L.: Gene expression patterns of breast carcinomas distinguish tumor subclasses with clinical implications. Proceedings of the National Academy of Sciences of the United States of America 98(19) (2001) 10869-10874
12. Sorlie, T., Tibshirani, R., Parker, J., Hastie, T., Marron, J. S., Nobel, A., Deng, S., Johnsen, H., Pesich, R., Geisler, S., Demeter, J., Perou, C. M., Lonning, P. E., Brown, P. O., Borresen-Dale, A. L., Botstein, D.: Repeated observation of breast tumor subtypes in independent gene expression data sets. Proceedings of the National Academy of Sciences of the United States of America 100(14) (2003) 8418-8423
13. Sotiriou, C., Neo, S. Y., McShane, L. M., Korn, E. L., Long, P. M., Jazaeri, A., Martiat, P., Fox, S. B., Harris, A. L., Liu, E. T.: Breast cancer classification and prognosis based on gene expression profiles from a population-based study. Proceedings of the National Academy of Sciences of the United States of America 100(18) (2003) 10393-10398
14. van de Vijver, M. J., He, Y. D., van der Veer, L. J., Dai, H., Hart, A. A. M., Voskuil, D. W., Schreiber, G. J., Peterse, J. L., Roberts, C., Marton, M. J., Parrish, M., Atsma, D., Witteveen, A., Glas, A., Delahaye, L., van der Velde, T., Bartelink, H., Rodenhuis, S., Rutgers, E. T., Friend, S. H., Bernards, R.: A gene-expression signature as a predictor of survival in breast cancer. New England Journal of Medicine 347(25) (2002) 1999-2009
15. van't Veer, L. J., Dai, H. Y., van de Vijver, M. J., He, Y. D. D., Hart, A. A. M., Mao, M., Peterse, H. L., van der Kooy, K., Marton, M. J., Witteveen, A. T., Schreiber, G. J., Kerkhoven, R. M., Roberts, C., Linsley, P. S., Bernards, R., Friend, S. H.: Gene expression profiling predicts clinical outcome of breast cancer. Nature 415(6871) (2002) 530-536
16. Cunha, G. R., Hayward, S. W., Wang, Y. Z., Ricke, W. A.: Role of the stromal microenvironment in carcinogenesis of the prostate. International Journal of Cancer 107(1) (2003) 1-10
17. Jia, Z., Wang, Y., Koziol, J., McClelland, M., Mercola, D.: A new bi-model classifier for predicting outcomes of prostate cancer patients. in JSM Proceedings, Biometrics Section. Denver, Colo.: American Statistical Association. (2008)

TABLE 33
Prognostic prostate cancer genes (biomarkers) in stroma cells identified by t-test following triage of training cases based on
calculated low tumor cell percentage

| | Probe.Set.ID | Gene.Title |
|---|---|---|
| 9212 | 209724_s_at | zinc finger protein 161 homolog (mouse) |
| 8569 | 209075_s_at | iron-sulfur cluster scaffold homolog (E. coli) |
| 5558 | 206031_s_at | ubiquitin specific peptidase 5 (isopeptidase T) |
| 2137 | 202609_at | epidermal growth factor receptor pathway substrate 8 |
| 17587 | 218222_x_at | aryl hydrocarbon receptor nuclear translocator |
| 20870 | 221507_at | transportin 2 (importin 3, karyopherin beta 2b) |
| 3319 | 203792_x_at | polycomb group ring finger 2 |
| 254 | 200726_at | protein phosphatase 1, catalytic subunit, gamma isoform |
| 687 | 201159_s_at | N-myristoyltransferase 1 |
| 18431 | 219067_s_at | non-SMC element 4 homolog A (S. cerevisiae) |
| 9148 | 209659_s_at | cell division cycle 16 homolog (S. cerevisiae) |
| 10469 | 211023_at | pyruvate dehydrogenase (lipoamide) beta |
| 21176 | 221816_s_at | PHD finger protein 11 |
| 3636 | 204109_s_at | nuclear transcription factor Y, alpha |
| 11450 | 212064_x_at | MYC-associated zinc finger protein (purine-binding transcription factor) |
| 4295 | 204768_s_at | flap structure-specific endonuclease 1 |
| 12711 | 213330_s_at | stress-induced-phosphoprotein 1 (Hsp70/Hsp90-organizing protein) |
| 18080 | 218716_x_at | mitochondrial translation optimization 1 homolog (S. cerevisiae) |
| 728 | 201200_at | cellular repressor of E1A-stimulated genes 1 |
| 1825 | 202297_s_at | RER1 retention in endoplasmic reticulum 1 homolog (S. cerevisiae) |
| 18419 | 219055_at | S1 RNA binding domain 1 |
| 3811 | 204284_at | protein phosphatase 1, regulatory (inhibitor) subunit 3C |
| 8782 | 209288_s_at | CDC42 effector protein (Rho GTPase binding) 3 |
| 12103 | 212718_at | poly(A) polymerase alpha |
| 3791 | 204264_at | carnitine palmitoyltransferase II |
| 17188 | 217823_s_at | ubiquitin-conjugating enzyme E2, J1 (UBC6 homolog, yeast) |
| 21817 | 34868_at | Smg-5 homolog, nonsense mediated mRNA decay factor (C. elegans) |
| 12250 | 212865_s_at | collagen, type XIV, alpha 1 |
| 11396 | 212009_s_at | stress-induced-phosphoprotein 1 (Hsp70/Hsp90-organizing protein) |
| 11407 | 212021_s_at | antigen identified by monoclonal antibody Ki-67 |
| 21773 | 32541_at | protein phosphatase 3 (formerly 2B), catalytic subunit, gamma isoform |
| 15404 | 216032_s_at | ERGIC and golgi 3 |
| 2460 | 202931_x_at | bridging integrator 1 |
| 17360 | 217995_at | sulfide quinone reductase-like (yeast) |
| 8725 | 209231_s_at | dynactin 5 (p25) |
| 21295 | 221935_s_at | chromosome 3 open reading frame 64 |
| 22178 | 65517_at | adaptor-related protein complex 1, mu 2 subunit |
| 20785 | 221422_s_at | chromosome 9 open reading frame 45 |
| 17290 | 217925_s_at | chromosome 6 open reading frame 106 |
| 2905 | 203378_at | PCF11, cleavage and polyadenylation factor subunit, homolog (S. cerevisiae) |
| 14114 | 214738_s_at | NIMA (never in mitosis gene a)-related kinase 9 |
| 2706 | 203178_at | glycine amidinotransferase (L-arginine:glycine amidinotransferase) |
| 19211 | 219847_at | histone deacetylase 11 |
| 17855 | 218490_s_at | zinc finger protein 302 |
| 10113 | 210648_x_at | sorting nexin 3 |
| 20886 | 221523_s_at | Ras-related GTP binding D |
| 11565 | 212179_at | splicing factor, arginine/serine-rich 18 |
| 19134 | 219770_at | glycosyltransferase-like domain containing 1 |
| 5199 | 205672_at | xeroderma pigmentosum, complementation group A |
| 3167 | 203640_at | muscleblind-like 2 (Drosophila) |
| 10433 | 210986_s_at | tropomyosin 1 (alpha) |
| 88 | 200067_x_at | sorting nexin 3 |
| 13818 | 214439_x_at | bridging integrator 1 |
| 2399 | 202871_at | TNF receptor-associated factor 4 |
| 11570 | 212184_s_at | mitogen-activated protein kinase kinase kinase 7 interacting protein 2 |
| 9418 | 209932_s_at | deoxyuridine triphosphatase |
| 21148 | 221788_at | CDNA FLJ11614 fis, clone HEMBA1004015 |
| 12476 | 213093_at | protein kinase C, alpha |
| 13966 | 214588_s_at | Microfibrillar-associated protein 3 |
| 2851 | 203324_s_at | caveolin 2 |
| 21207 | 221847_at | hypothetical protein LOC100129361 |
| 18159 | 218795_at | acid phosphatase 6, lysophosphatidic |
| 11533 | 212147_at | Smg-5 homolog, nonsense mediated mRNA decay factor (C. elegans) |
| 873 | 201345_s_at | ubiquitin-conjugating enzyme E2D 2 (UBC4/5 homolog, yeast) |
| 14634 | 215260_s_at | transcription factor 3 (E2A immunoglobulin enhancer binding factors E12/E47) |
| 16339 | 216969_s_at | kinesin family member 22 |
| 12895 | 213514_s_at | diaphanous homolog 1 (Drosophila) |
| 1911 | 202383_at | jumonji, AT rich interactive domain 1C |
| 11497 | 212111_at | syntaxin 12 |
| 4074 | 204547_at | RAB40B, member RAS oncogene family |
| 19713 | 220349_s_at | endo-beta-N-acetylglucosaminidase |
| 6528 | 207002_s_at | pleiomorphic adenoma gene-like 1 |
| 17271 | 217906_at | kelch domain containing 2 |
| 7906 | 208405_s_at | CD164 molecule, sialomucin |
| 9685 | 210201_x_at | bridging integrator 1 |
| 12557 | 213175_s_at | small nuclear ribonucleoprotein polypeptides B and B1 |
| 5636 | 206110_at | histone cluster 1, H3h |
| 3411 | 203884_s_at | RAB11 family interacting protein 2 (class I) |
| 795 | 201267_s_at | proteasome (prosome, macropain) 26S subunit, ATPase, 3 |
| 4490 | 204963_at | sarcospan (Kras oncogene-associated gene) |
| 14375 | 215000_s_at | fasciculation and elongation protein zeta 2 (zygin II) |
| 21934 | 39549_at | neuronal PAS domain protein 2 |
| 9513 | 210028_s_at | origin recognition complex, subunit 3-like (yeast) |
| 14256 | 214881_s_at | upstream binding transcription factor, RNA polymerase I |
| 9676 | 210192_at | ATPase, aminophospholipid transporter (APLT), class I, type 8A, member 1 |
| 17714 | 218349_s_at | Zwilch, kinetochore associated, homolog (Drosophila) |
| 758 | 201230_s_at | ariadne homolog 2 (Drosophila) |
| 6748 | 207223_s_at | ROD1 regulator of differentiation 1 (S. pombe) |
| 11624 | 212238_at | additional sex combs like 1 (Drosophila) |
| 9009 | 209516_at | SMYD family member 5 |
| 9763 | 210283_x_at | poly(A) binding protein interacting protein 1 /// hypothetical LOC645139 /// similar |
| | | to poly(A) binding protein interacting protein 1 isoform |
| 2347 | 202819_s_at | transcription elongation factor B (SIII), polypeptide 3 (110 kDa, elongin A) |
| 3641 | 204114_at | nidogen 2 (osteonidogen) |
| 17544 | 218179_s_at | chromosome 4 open reading frame 41 |
| 2420 | 202892_at | cell division cycle 23 homolog (S. cerevisiae) |
| 17880 | 218515_at | chromosome 21 open reading frame 66 |
| 12084 | 212699_at | secretory carrier membrane protein 5 |
| 18062 | 218698_at | APAF1 interacting protein |
| 5138 | 205611_at | tumor necrosis factor (ligand) superfamily, member 12 |
| 8201 | 208706_s_at | eukaryotic translation initiation factor 5 |
| 13554 | 214175_x_at | PDZ and LIM domain 4 |
| 4466 | 204939_s_at | phospholamban |
| 8451 | 208956_x_at | deoxyuridine triphosphatase |
| 10085 | 210620_s_at | general transcription factor IIIC, polypeptide 2, beta 110 kDa |
| 17458 | 218093_s_at | ankyrin repeat domain 10 |
| 19049 | 219685_at | transmembrane protein 35 |
| 20799 | 221436_s_at | cell division cycle associated 3 |
| 17196 | 217831_s_at | NSFL1 (p97) cofactor (p47) |
| 8707 | 209213_at | carbonyl reductase 1 |
| 11700 | 212315_s_at | nucleoporin 210 kDa |
| 12779 | 213398_s_at | chromosome 14 open reading frame 124 |
| 17874 | 218509_at | lipid phosphate phosphatase-related protein type 2 |
| 12018 | 212633_at | KIAA0776 |
| 11483 | 212097_at | caveolin 1, caveolae protein, 22 kDa |
| 11077 | 211675_s_at | MyoD family inhibitor domain containing |
| 13258 | 213878_at | Pyridine nucleotide-disulphide oxidoreductase domain 1 |
| 3045 | 203518_at | lysosomal trafficking regulator |
| 13715 | 214336_s_at | coatomer protein complex, subunit alpha |
| 6056 | 206530_at | RAB30, member RAS oncogene family |
| 21792 | 33760_at | peroxisomal biogenesis factor 14 |
| 12821 | 213440_at | RAB1A, member RAS oncogene family |
| 11882 | 212497_at | mitogen-activated protein kinase 1 interacting protein 1-like |
| 2181 | 202653_s_at | membrane-associated ring finger (C3HC4) 7 |
| 1361 | 201833_at | histone deacetylase 2 |
| 5330 | 205803_s_at | transient receptor potential cation channel, subfamily C, member 1 |
| 2493 | 202964_s_at | regulatory factor X, 5 (influences HLA class II expression) |
| 18531 | 219167_at | RAS-like, family 12 |
| 14074 | 214698_at | ROD1 regulator of differentiation 1 (S. pombe) |
| 7438 | 207922_s_at | macrophage erythroblast attacher |
| 17412 | 218047_at | oxysterol binding protein-like 9 |
| 2057 | 202529_at | phosphoribosyl pyrophosphate synthetase-associated protein 1 |
| 2857 | 203330_s_at | syntaxin 5 |
| 462 | 200934_at | DEK oncogene (DNA binding) |
| 11200 | 211804_s_at | cyclin-dependent kinase 2 |
| 535 | 201007_at | hydroxyacyl-Coenzyme A dehydrogenase/3-ketoacyl-Coenzyme |
| | | A thiolase/enoyl-Coenzyme A hydratase (trifunctional protein), beta |
| 3466 | 203939_at | 5′-nucleotidase, ecto (CD73) |
| 12354 | 212971_at | cysteinyl-tRNA synthetase |
| 1302 | 201774_s_at | non-SMC condensin I complex, subunit D2 |
| 3552 | 204025_s_at | programmed cell death 2 |
| 13816 | 214437_s_at | serine hydroxymethyltransferase 2 (mitochondrial) |
| 3313 | 203786_s_at | tumor protein D52-like 1 |
| 550 | 201022_s_at | destrin (actin depolymerizing factor) |
| 11942 | 212557_at | zinc finger protein 451 |
| 450 | 200922_at | KDEL (Lys-Asp-Glu-Leu) endoplasmic reticulum protein retention receptor 1 |
| 20636 | 221273_s_at | ring finger protein 208 /// similar to ring finger protein 208 |
| 2546 | 203017_s_at | synovial sarcoma, X breakpoint 2 interacting protein |
| 10425 | 210978_s_at | transgelin 2 |
| 20106 | 220742_s_at | N-glycanase 1 |
| 6380 | 206854_s_at | mitogen-activated protein kinase kinase kinase 7 |
| 12864 | 213483_at | peptidylprolyl isomerase domain and WD repeat containing 1 |
| 19458 | 220094_s_at | coiled-coil domain containing 90A |
| 4482 | 204955_at | sushi-repeat-containing protein, X-linked |
| 3927 | 204400_at | embryonal Fyn-associated substrate |
| 20553 | 221190_s_at | chromosome 18 open reading frame 8 |
| 14854 | 215481_s_at | peroxisomal biogenesis factor 5 |
| 9947 | 210470_x_at | non-POU domain containing, octamer-binding |
| 7458 | 207943_x_at | pleiomorphic adenoma gene-like 1 |
| 18479 | 219115_s_at | interleukin 20 receptor, alpha |
| 1794 | 202266_at | TRAF and TNF receptor associated protein |
| 18133 | 218769_s_at | ankyrin repeat, family A (RFXANK-like), 2 |
| 7033 | 207511_s_at | chromosome 2 open reading frame 24 |
| 11562 | 212176_at | splicing factor, arginine/serine-rich 18 |
| 4578 | 205051_s_at | v-kit Hardy-Zuckerman 4 feline sarcoma viral oncogene homolog |
| 1960 | 202432_at | protein phosphatase 3 (formerly 2B), catalytic subunit, beta isoform |
| 7579 | 208070_s_at | REV3-like, catalytic subunit of DNA polymerase zeta (yeast) |
| 1655 | 202127_at | PRP4 pre-mRNA processing factor 4 homolog B (yeast) |
| 14198 | 214823_at | zinc finger protein 204 (pseudogene) |
| 4467 | 204940_at | phospholamban |
| 19299 | 219935_at | ADAM metallopeptidase with thrombospondin type 1 motif, 5 (aggrecanase-2) |
| 12388 | 213005_s_at | KN motif and ankyrin repeat domains 1 |
| 3233 | 203706_s_at | frizzled homolog 7 (Drosophila) |
| 16813 | 217448_s_at | TOX high mobility group box family member 4 /// similar to KIAA0737 protein |
| 20865 | 221502_at | karyopherin alpha 3 (importin alpha 4) |
| 11630 | 212244_at | glutamate receptor, ionotropic, N-methyl D-aspartate-like 1A /// GRINL1A combined protein |
| 1593 | 202065_s_at | protein tyrosine phosphatase, receptor type, f polypeptide (PTPRF), interacting protein (liprin), alpha 1 |
| 8726 | 209232_s_at | dynactin 5 (p25) |
| 17131 | 217766_s_at | transmembrane protein 50A |
| 3776 | 204249_s_at | LIM domain only 2 (rhombotin-like 1) |
| 7785 | 208281_x_at | deleted in azoospermia 1 /// deleted in azoospermia 3 /// deleted in |
| | | azoospermia 2 /// deleted in azoospermia 4 /// similar to deleted in a [symbol] like |
| 17228 | 217863_at | protein inhibitor of activated STAT, 1 |
| 14501 | 215127_s_at | RNA binding motif, single stranded interacting protein 1 |
| 13906 | 214527_s_at | polyglutamine binding protein 1 |
| 12674 | 213293_s_at | tripartite motif-containing 22 |
| 6464 | 206938_at | steroid-5-alpha-reductase, alpha polypeptide 2 (3-oxo-5 alpha-steroid delta 4-dehydrogenase alpha 2) |
| 2711 | 203183_s_at | SWI/SNF related, matrix associated, actin dependent regulator of chromatin, subfamily d, member 1 |
| 12083 | 212698_s_at | septin 10 |
| 9042 | 209550_at | necdin homolog (mouse) |
| 11083 | 211681_s_at | PDZ and LIM domain 5 |
| 20841 | 221478_at | BCL2/adenovirus E1B 19 kDa interacting protein 3-like |
| 18981 | 219617_at | chromosome 2 open reading frame 34 |
| 13702 | 214323_s_at | UPF3 regulator of nonsense transcripts homolog A (yeast) |
| 8662 | 209168_at | glycoprotein M6B |
| 13151 | 213771_at | interferon regulatory factor 2 binding protein 1 |
| 20946 | 221584_s_at | potassium large conductance calcium-activated channel, subfamily M, alpha member 1 |
| 1131 | 201603_at | protein phosphatase 1, regulatory (inhibitor) subunit 12A |
| 20510 | 221147_x_at | WW domain containing oxidoreductase |
| 14312 | 214937_x_at | pericentriolar material 1 |
| 19162 | 219798_s_at | methylphosphate capping enzyme |
| 20996 | 221634_at | ribosomal protein L23a pseudogene 7 |
| 17452 | 218087_s_at | sorbin and SH3 domain containing 1 |
| 975 | 201447_at | TIA1 cytotoxic granule-associated RNA binding protein |
| 3991 | 204464_s_at | endothelin receptor type A |
| 4563 | 205036_at | LSM6 homolog, U6 small nuclear RNA associated (S. cerevisiae) |
| 19141 | 219777_at | GTPase, IMAP family member 6 |
| 11488 | 212102_s_at | karyopherin alpha 6 (importin alpha 7) |
| 1730 | 202202_s_at | laminin, alpha 4 |
| 6437 | 206911_at | tripartite motif-containing 25 |
| 15666 | 216294_s_at | KIAA1109 |
| 2220 | 202692_s_at | upstream binding transcription factor, RNA polymerase I |
| 8786 | 209292_at | Inhibitor of DNA binding 4, dominant negative helix-loop-helix protein |
| 1846 | 202318_s_at | SUMO1/sentrin specific peptidase 6 |
| 12643 | 213262_at | spastic ataxia of Charlevoix-Saguenay (sacsin) |
| 12288 | 212904_at | leucine rich repeat containing 47 |
| 5630 | 206104_at | ISL LIM homeobox 1 |
| 15760 | 216389_s_at | WD repeat domain 23 |
| 3217 | 203690_at | tubulin, gamma complex associated protein 3 |
| 1721 | 202193_at | LIM domain kinase 2 |
| 12866 | 213485_s_at | ATP-binding cassette, sub-family C (CFTR/MRP), member 10 |
| 18742 | 219378_at | NMDA receptor regulated 1-like |
| 15919 | 216549_s_at | TBC1 domain family, member 22B |
| 3932 | 204405_x_at | DIM1 dimethyladenosine transferase 1-like (S. cerevisiae) |
| 12080 | 212695_at | cryptochrome 2 (photolyase-like) |
| 12365 | 212982_at | zinc finger, DHHC-type containing 17 |
| 14210 | 214835_s_at | succinate-CoA ligase, GDP-forming, beta subunit |
| 8870 | 209377_s_at | high mobility group nucleosomal binding domain 3 |
| 4427 | 204900_x_at | Sin3A-associated protein, 30 kDa |
| 2850 | 203323_at | caveolin 2 |
| 3965 | 204438_at | mannose receptor, C type 1 /// mannose receptor, C type 1-like 1 |
| 17047 | 217682_at | CDNA FLJ37032 fis, clone BRACE2011265 |
| 1661 | 202133_at | WW domain containing transcription regulator 1 |
| 17157 | 217792_at | sorting nexin 5 |
| 18811 | 219447_s_at | solute carrier family 35, member C2 /// hypothetical protein LOC100128167 |
| 1890 | 202362_at | RAP1A, member of RAS oncogene family |
| 10969 | 211564_s_at | PDZ and LIM domain 4 |
| 11680 | 212294_at | guanine nucleotide binding protein (G protein), gamma 12 |
| 1095 | 201567_s_at | golgi autoantigen, golgin subfamily a, 4 |
| 8812 | 209318_x_at | pleiomorphic adenoma gene-like 1 |
| 2833 | 203306_s_at | solute carrier family 35 (CMP-sialic acid transporter), member A1 |
| 4220 | 204693_at | CDC42 effector protein (Rho GTPase binding) 1 |
| 5568 | 206042_x_at | small nuclear ribonucleoprotein polypeptide N /// SNRPN upstream reading frame |
| 20179 | 220815_at | catenin (cadherin-associated protein), alpha 3 |
| 279 | 200751_s_at | heterogeneous nuclear ribonucleoprotein C (C1/C2) |
| 12687 | 213306_at | multiple PDZ domain protein |
| 9307 | 209821_at | interleukin 33 |
| 18058 | 218694_at | armadillo repeat containing, X-linked 1 |
| 1678 | 202150_s_at | neural precursor cell expressed, developmentally down-regulated 9 |
| 11506 | 212120_at | ras homolog gene family, member Q |

[symbol]

indicates data missing or illegible when filed

TABLE 34
Prognostic prostate cancer genes (biomarkers) in stroma cells identified by t-test following triage of training cases based on
calculated low stroma cell percentage

| | Probe.Set.ID | Gene.Title | Gene.Symbol |
|---|---|---|---|
| 4409 | 204882_at | Rho GTPase activating protein 25 | ARHGAP25 |
| 10218 | 210757_x_at | disabled homolog 2, mitogen-responsive phosphoprotein (Drosophila) | DAB2 |
| 12214 | 212829_at | phosphatidylinositol-5-phosphate 4-kinase, type II, alpha | PIP4K2A |
| 5360 | 205833_s_at | prostate androgen-regulated transcript 1 | PART1 |
| 597 | 201069_at | matrix metallopeptidase 2 (gelatinase A, 72 kDa gelatinase, 72 kDa | MMP2 |
| | | type IV collagenase) | |
| 2486 | 202957_at | hematopoietic cell-specific Lyn substrate 1 | HCLS1 |
| 747 | 201219_at | C-terminal binding protein 2 | CTBP2 |
| 4090 | 204563_at | selectin L (lymphocyte adhesion molecule 1) | SELL |
| 807 | 201279_s_at | disabled homolog 2, mitogen-responsive phosphoprotein (Drosophila) | DAB2 |
| 13281 | 213902_at | N-acylsphingosine amidohydrolase (acid ceramidase) 1 | ASAH1 |
| 2887 | 203360_s_at | c-myc binding protein | MYCBP |
| 17122 | 217757_at | alpha-2-macroglobulin | A2M |
| 4389 | 204862_s_at | non-metastatic cells 3, protein expressed in | NME3 |
| 18011 | 218647_s_at | yrdC domain containing (E. coli) | YRDC |
| 12983 | 213603_s_at | ras-related C3 botulinum toxin substrate 2 (rho family, small GTP | RAC2 |
| | | binding protein Rac2) | |
| 17155 | 217790_s_at | signal sequence receptor, gamma (translocon-associated protein | SSR3 |
| | | gamma) | |
| 4797 | 205270_s_at | lymphocyte cytosolic protein 2 (SH2 domain containing leukocyte protein of 76 | LCP2 |
| | | kDa) | |
| 12129 | 212744_at | Bardet-Biedl syndrome 4 | BBS4 |
| 19941 | 220577_at | GTPase, very large interferon inducible 1 | GVIN1 |
| 2193 | 202665_s_at | WAS/WASL interacting protein family, member 1 | WIPF1 |
| 11688 | 212302_at | Rtf1, Paf1/RNA polymerase II complex component, homolog (S. cerevisiae) | RTF1 |
| 6383 | 206857_s_at | FK506 binding protein 1B, 12.6 kDa | FKBP1B |
| 2859 | 203332_s_at | inositol polyphosphate-5-phosphatase, 145 kDa | INPP5D |
| 514 | 200986_at | serpin peptidase inhibitor, clade G (C1 inhibitor), member 1, | SERPING1 |
| | | (angioedema, hereditary) | |
| 18285 | 218921_at | single immunoglobulin and toll-interleukin 1 receptor (TIR) domain | SIGIRR |
| 2957 | 203430_at | heme binding protein 2 | HEBP2 |
| 20298 | 220934_s_at | hypothetical protein MGC3196 | MGC3196 |
| 9589 | 210105_s_at | FYN oncogene related to SRC, FGR, YES | FYN |
| 4178 | 204651_at | nuclear respiratory factor 1 | NRF1 |
| 1133 | 201605_x_at | calponin 2 | CNN2 |
| 9182 | 209694_at | 6-pyruvoyltetrahydropterin synthase | PTS |
| 114 | 200093_s_at | histidine triad nucleotide binding protein 1 | HINT1 |
| 21957 | 40420_at | serine/threonine kinase 10 | STK10 |
| 4603 | 205076_s_at | myotubularin related protein 11 | MTMR11 |
| 4818 | 205291_at | interleukin 2 receptor, beta | IL2RB |
| 3702 | 204175_at | zinc finger protein 593 | ZNF593 |
| 128 | 200600_at | moesin | MSN |
| 2717 | 203189_s_at | NADH dehydrogenase (ubiquinone) Fe—S protein 8, 23 kDa (NADH- | NDUFS8 |
| | | coenzyme Q reductase) | |
| 12130 | 212745_s_at | Bardet-Biedl syndrome 4 | BBS4 |
| 15405 | 216033_s_at | FYN oncogene related to SRC, FGR, YES | FYN |
| 12384 | 213001_at | angiopoietin-like 2 | ANGPTL2 |
| 20618 | 221255_s_at | transmembrane protein 93 | TMEM93 |
| 1249 | 201721_s_at | lysosomal associated multispanning membrane protein 5 | LAPTM5 |
| 481 | 200953_s_at | cyclin D2 | CCND2 |
| 3822 | 204295_at | surfeit 1 | SURF1 |
| 21049 | 221688_s_at | IMP3, U3 small nucleolar ribonucleoprotein, homolog (yeast) | IMP3 |
| 17527 | 218162_at | olfactomedin-like 3 | OLFML3 |
| 17449 | 218084_x_at | FXYD domain containing ion transport regulator 5 | FXYD5 |
| 11705 | 212320_at | tubulin, beta | TUBB |
| 9039 | 209546_s_at | apolipoprotein L, 1 | APOL1 |
| 1955 | 202427_s_at | brain protein 44 | BRP44 |
| 21014 | 221653_x_at | apolipoprotein L, 2 | APOL2 |
| 4439 | 204912_at | interleukin 10 receptor, alpha | IL10RA |
| 11060 | 211656_x_at | major histocompatibility complex, class II, DQ beta 1 | HLA-DQB1 |
| 2458 | 202929_s_at | D-dopachrome tautomerase | DDT |
| 1824 | 202296_s_at | RER1 retention in endoplasmic reticulum 1 homolog (S. cerevisiae) | RER1 |
| 9159 | 209670_at | T cell receptor alpha constant | TRAC |
| 9247 | 209759_s_at | dodecenoyl-Coenzyme A delta isomerase (3,2 trans-enoyl-Coenzyme | DCI |
| | | A isomerase) | |
| 6394 | 206868_at | StAR-related lipid transfer (START) domain containing 8 | STARD8 |
| 3190 | 203663_s_at | cytochrome c oxidase subunit Va | COX5A |
| 5676 | 206150_at | CD27 molecule | CD27 |
| 3846 | 204319_s_at | regulator of G-protein signaling 10 | RGS10 |
| 12542 | 213159_at | pecanex homolog (Drosophila) | PCNX |
| 3724 | 204197_s_at | runt-related transcription factor 3 | RUNX3 |
| 18737 | 219373_at | dolichyl-phosphate mannosyltransferase polypeptide 3 | DPM3 |
| 3213 | 203686_at | N-methylpurine-DNA glycosylase | MPG |
| 21576 | 222216_s_at | mitochondrial ribosomal protein L17 | MRPL17 |
| 2576 | 203047_at | serine/threonine kinase 10 | STK10 |
| 451 | 200923_at | lectin, galactoside-binding, soluble, 3 binding protein | LGALS3BP |
| 1353 | 201825_s_at | saccharopine dehydrogenase (putative) | SCCPDH |
| 2331 | 202803_s_at | integrin, beta 2 (complement component 3 receptor 3 and 4 subunit) | ITGB2 |
| 21927 | 38964_r_at | Wiskott-Aldrich syndrome (eczema-thrombocytopenia) | WAS |
| 10103 | 210638_s_at | F-box protein 9 | FBXO9 |
| 510 | 200982_s_at | annexin A6 | ANXA6 |
| 12098 | 212713_at | microfibrillar-associated protein 4 | MFAP4 |
| 9109 | 209619_at | CD74 molecule, major histocompatibility complex, class II invariant | CD74 |
| | | chain | |
| 19176 | 219812_at | poliovirus receptor related immunoglobulin domain containing | PVRIG |
| 10245 | 210785_s_at | chromosome 1 open reading frame 38 | C1orf38 |
| 1194 | 201666_at | TIMP metallopeptidase inhibitor 1 | TIMP1 |
| 11431 | 212045_at | golgi apparatus protein 1 | GLG1 |
| 21908 | 38149_at | Rho GTPase activating protein 25 | ARHGAP25 |
| 4322 | 204795_at | proline rich 3 | PRR3 |
| 11729 | 212344_at | sulfatase 1 | SULF1 |
| 17946 | 218581_at | abhydrolase domain containing 4 | ABHD4 |
| 13115 | 213735_s_at | cytochrome c oxidase subunit Vb | COX5B |
| 1286 | 201758_at | tumor susceptibility gene 101 | TSG101 |
| 69 | 200048_s_at | jumping translocation breakpoint | JTB |
| 12936 | 213555_at | RWD domain containing 2A | RWDD2A |
| 12175 | 212790_x_at | ribosomal protein L13a | RPL13A |
| 374 | 200846_s_at | protein phosphatase 1, catalytic subunit, alpha isoform | PPP1CA |
| 4627 | 205100_at | glutamine-fructose-6-phosphate transaminase 2 | GFPT2 |
| 19796 | 220432_s_at | cytochrome P450, family 39, subfamily A, polypeptide 1 | CYP39A1 |
| 12270 | 212885_at | M-phase phosphoprotein 10 (U3 small nucleolar ribonucleoprotein) | MPHOSPH10 |
| 8321 | 208826_x_at | histidine triad nucleotide binding protein 1 | HINT1 |
| 19040 | 219676_at | zinc finger and SCAN domain containing 16 | ZSCAN16 |
| 3913 | 204386_s_at | mitochondrial ribosomal protein 63 | MRP63 |
| 3739 | 204212_at | acyl-CoA thioesterase 8 | ACOT8 |
| 9791 | 210312_s_at | intraflagellar transport 20 homolog (Chlamydomonas) | IFT20 |
| 222 | 200694_s_at | DEAD (Asp-Glu-Ala-Asp) box polypeptide 24 | DDX24 |
| 22079 | 52169_at | protein kinase LYK5 | LYK5 |
| 20810 | 221447_s_at | glycosyltransferase 8 domain containing 2 | GLT8D2 |
| 8975 | 209482_at | processing of precursor 7, ribonuclease P/MRP subunit (S. cerevisiae) | POP7 |
| 2633 | 203104_at | colony stimulating factor 1 receptor, formerly McDonough feline | CSF1R |
| | | sarcoma viral (v-fms) oncogene homolog | |
| 2895 | 203368_at | cysteine-rich with EGF-like domains 1 | CRELD1 |
| 12961 | 213581_at | programmed cell death 2 | PDCD2 |
| 4450 | 204923_at | SAM and SH3 domain containing 3 | SASH3 |
| 4703 | 205176_s_at | integrin beta 3 binding protein (beta3-endonexin) | ITGB3BP |
| 17623 | 218258_at | polymerase (RNA) I polypeptide D, 16 kDa | POLR1D |
| 954 | 201426_s_at | vimentin | VIM |
| 4538 | 205011_at | loss of heterozygosity, 11, chromosomal region 2, gene A | LOH11CR2A |
| 1248 | 201720_s_at | lysosomal associated multispanning membrane protein 5 | LAPTM5 |
| 2617 | 203088_at | fibulin 5 | FBLN5 |
| 5085 | 205558_at | TNF receptor-associated factor 6 | TRAF6 |
| 9115 | 209625_at | phosphatidylinositol glycan anchor biosynthesis, class H | PIGH |
| 9095 | 209605_at | thiosulfate sulfurtransferase (rhodanese) | TST |
| 1096 | 201568_at | ubiquinol-cytochrome c reductase, complex III subunit VII, 9.5 kDa | UQCRQ |
| 2799 | 203272_s_at | tumor suppressor candidate 2 | TUSC2 |
| 17368 | 218003_s_at | FK506 binding protein 3, 25 kDa | FKBP3 |
| 13622 | 214243_s_at | serine hydrolase-like /// serine hydrolase-like 2 | SERHL /// SERHL2 |
| 7068 | 207547_s_at | family with sequence similarity 107, member A | FAM107A |
| 3000 | 203473_at | solute carrier organic anion transporter family, member 2B1 | SLCO2B1 |
| 5592 | 206066_s_at | RAD51 homolog C (S. cerevisiae) | RAD51C |
| 7810 | 208306_x_at | Major histocompatibility complex, class II, DR beta 3 | HLA-DRB1 |
| 17928 | 218563_at | NADH dehydrogenase (ubiquinone) 1 alpha subcomplex, 3, 9 kDa | NDUFA3 |
| 3701 | 204174_at | arachidonate 5-lipoxygenase-activating protein | ALOX5AP |
| 20998 | 221637_s_at | chromosome 11 open reading frame 48 | C11orf48 |
| 5303 | 205776_at | flavin containing monooxygenase 5 | FMO5 |
| 16727 | 217362_x_at | major histocompatibility complex, class II, DR beta 6 (pseudogene) | HLA-DRB6 |
| 3005 | 203478_at | NADH dehydrogenase (ubiquinone) 1, subcomplex unknown, 1, | NDUFC1 |
| | | 6 kDa | |
| 329 | 200801_x_at | actin, beta | ACTB |
| 13476 | 214097_at | ribosomal protein S21 | RPS21 |
| 4521 | 204994_at | myxovirus (influenza virus) resistance 2 (mouse) | MX2 |
| 3837 | 204310_s_at | natriuretic peptide receptor B/guanylate cyclase B (atrionatriuretic | NPR2 |
| | | peptide receptor B) | |
| 2052 | 202524_s_at | sparc/osteonectin, cwcv and kazal-like domains proteoglycan | SPOCK2 |
| | | (testican) 2 | |
| 8796 | 209302_at | polymerase (RNA) II (DNA directed) polypeptide H | POLR2H |
| 18643 | 219279_at | dedicator of cytokinesis 10 | DOCK10 |
| 8695 | 209201_x_at | chemokine (C—X—C motif) receptor 4 | CXCR4 |
| 1931 | 202403_s_at | collagen, type I, alpha 2 | COL1A2 |
| 1711 | 202183_s_at | kinesin family member 22 | KIF22 |
| 1481 | 201953_at | calcium and integrin binding 1 (calmyrin) | CIB1 |
| 453 | 200925_at | cytochrome c oxidase subunit VIa polypeptide 1 | COX6A1 |
| 17794 | 218429_s_at | hypothetical protein FLJ11286 | FLJ11286 |
| 3262 | 203735_x_at | PTPRF interacting protein, binding protein 1 (liprin beta 1) | PPFIBP1 |
| 18482 | 219118_at | FK506 binding protein 11, 19 kDa | FKBP11 |
| 209 | 200681_at | glyoxalase I | GLO1 |
| 2832 | 203305_at | coagulation factor XIII, A1 polypeptide | F13A1 |
| 17945 | 218580_x_at | aurora kinase A interacting protein 1 | AURKAIP1 |
| 12551 | 213169_at | sema domain, seven thrombospondin repeats (type 1 and type 1-like), | SEMA5A |
| | | transmembrane domain (TM) and short cytoplasmic domain, | |
| | | (semaphorin) 5A | |
| 9322 | 209836_x_at | bolA homolog 2 (E. coli) /// bolA homolog 2B (E. coli) | BOLA2 /// BOLA2B |
| 988 | 201460_at | mitogen-activated protein kinase-activated protein kinase 2 | MAPKAPK2 |
| 19126 | 219762_s_at | ribosomal protein L36 | RPL36 |
| 3380 | 203853_s_at | GRB2-associated binding protein 2 | GAB2 |
| 3963 | 204436_at | pleckstrin homology domain containing, family O member 2 | PLEKHO2 |
| 16485 | 217118_s_at | chromosome 22 open reading frame 9 | C22orf9 |
| 43 | 200022_at | ribosomal protein L18 | RPL18 |
| 21435 | 222075_s_at | ornithine decarboxylase antizyme 3 | OAZ3 |
| 9014 | 209521_s_at | angiomotin | AMOT |
| 5307 | 205780_at | BCL2-interacting killer (apoptosis-inducing) | BIK |
| 9098 | 209608_s_at | acetyl-Coenzyme A acetyltransferase 2 | ACAT2 |
| 13165 | 213785_at | importin 9 | IPO9 |
| 18169 | 218805_at | GTPase, IMAP family member 5 | GIMAP5 |
| 1320 | 201792_at | AE binding protein 1 | AEBP1 |
| 21338 | 221978_at | major histocompatibility complex, class I, F | HLA-F |
| 20797 | 221434_s_at | chromosome 14 open reading frame 156 | C14orf156 |
| 12496 | 213113_s_at | solute carrier family 43, member 3 | SLC43A3 |
| 3838 | 204311_at | ATPase, Na+/K+ transporting, beta 2 polypeptide | ATP1B2 |
| 10333 | 210879_s_at | RAB11 family interacting protein 5 (class I) | RAB11FIP5 |
| 1268 | 201740_at | NADH dehydrogenase (ubiquinone) Fe—S protein 3, 30 kDa (NADH- | NDUFS3 |
| | | coenzyme Q reductase) | |
| 13374 | 213995_at | ATP synthase, H+ transporting, mitochondrial F0 complex, subunit s | ATP5S |
| | | (factor B) | |
| 2559 | 203030_s_at | protein tyrosine phosphatase, receptor type, N polypeptide 2 | PTPRN2 |
| 19115 | 219751_at | SET domain containing 6 | SETD6 |
| 1811 | 202283_at | serpin peptidase inhibitor, clade F (alpha-2 antiplasmin, pigment | SERPINF1 |
| | | epithelium derived factor), member 1 | |
| 9721 | 210241_s_at | TP53 activated protein 1 | TP53AP1 |
| 20821 | 221458_at | 5-hydroxytryptamine (serotonin) receptor 1F | HTR1F |
| 570 | 201042_at | transglutaminase 2 (C polypeptide, protein-glutamine-gamma- | TGM2 |
| | | glutamyltransferase) | |
| 143 | 200615_s_at | adaptor-related protein complex 2, beta 1 subunit | AP2B1 |
| 22228 | AFFX- | actin, beta | ACTB |
| | HSAC07/X00351_3_at | | |
| 11555 | 212169_at | FK506 binding protein 9, 63 kDa | FKBP9 |
| 2964 | 203437_at | transmembrane protein 11 | TMEM11 |
| 12381 | 212998_x_at | major histocompatibility complex, class II, DQ beta 1 /// major | hCG_1998957 /// HLA-DQB1 /// |
| | | histocompatibility complex, class II, DQ beta 2 /// major | HLA-DQB2 /// HLA-DRB1 /// |
| | | histocompatibility complex, class II, DR beta 1 /// major | HLA-DRB2 /// HLA-DRB3 /// |
| | | histocompatibility complex, class II, DR beta 2 (pseudogene) /// | HLA-DRB4 /// HLA-DRB5 /// |
| | | major histocompatibility complex, class II, DR beta 3 /// major | LOC100133484 /// |
| | | histocompatibility complex, class II, DR beta 4 /// major | LOC100133583 /// |
| | | histocompatibility complex, class II, DR beta 5 /// ribonuclease, | LOC100133661 /// |
| | | RNase A family, 2 (liver, eosinophil-derived neurotoxin) /// zinc | LOC100133811 /// LOC730415 /// |
| | | finger protein 749 /// hypothetical protein LOC730415 /// similar to | RNASE2 /// ZNF749 |
| | | Major histocompatibility complex, class II, DR beta 4 /// similar to | |
| | | major histocompatibility complex, class II, DQ beta 1 /// similar to | |
| | | HLA class II histocompatibility antigen, DR-W53 beta chain /// | |
| | | similar to hCG1992647 | |
| 17360 | 217995_at | sulfide quinone reductase-like (yeast) | SQRDL |
| 3867 | 204340_at | transmembrane protein 187 | TMEM187 |
| 10757 | 211339_s_at | IL2-inducible T-cell kinase | ITK |
| 3858 | 204331_s_at | mitochondrial ribosomal protein S12 | MRPS12 |
| 8838 | 209345_s_at | phosphatidylinositol 4-kinase type 2 alpha | PI4K2A |
| 3192 | 203665_at | heme oxygenase (decycling) 1 | HMOX1 |
| 12575 | 213193_x_at | T cell receptor beta constant 1 | TRBC1 |
| 18505 | 219141_s_at | autophagy/beclin-1 regulator 1 | AMBRA1 |
| 9864 | 210386_s_at | metaxin 1 | MTX1 |
| 3035 | 203508_at | tumor necrosis factor receptor superfamily, member 1B | TNFRSF1B |
| 2718 | 203190_at | NADH dehydrogenase (ubiquinone) Fe—S protein 8, 23 kDa (NADH- | NDUFS8 |
| | | coenzyme Q reductase) | |
| 16614 | 217249_x_at | cytochrome c oxidase subunit VIIa polypeptide 2 (liver) | COX7A2 |
| 347 | 200819_s_at | ribosomal protein S15 | RPS15 |
| 647 | 201119_s_at | cytochrome c oxidase subunit 8A (ubiquitous) | COX8A |
| 8598 | 209104_s_at | nucleolar protein family A, member 2 (H/ACA small nucleolar RNPs) | NOLA2 |
| 3832 | 204305_at | mitochondrial intermediate peptidase | MIPEP |
| 1083 | 201555_at | minichromosome maintenance complex component 3 | MCM3 |
| 18261 | 218897_at | transmembrane protein 177 | TMEM177 |
| 21091 | 221731_x_at | versican | VCAN |
| 9912 | 210434_x_at | jumping translocation breakpoint | JTB |
| 17597 | 218232_at | complement component 1, q subcomponent, A chain | C1QA |
| 290 | 200762_at | dihydropyrimidinase-like 2 | DPYSL2 |
| 8862 | 209369_at | annexin A3 | ANXA3 |
| 12835 | 213454_at | apoptosis-inducing, TAF9-like domain 1 | APITD1 |
| 2327 | 202799_at | ClpP caseinolytic peptidase, ATP-dependent, proteolytic subunit | CLPP |
| | | homolog (E. coli) | |
| 18314 | 218950_at | centaurin, delta 3 | CENTD3 |
| 70 | 200049_at | MYST histone acetyltransferase 2 | MYST2 |
| 8859 | 209366_x_at | cytochrome b5 type A (microsomal) | CYB5A |
| 8144 | 208647_at | farnesyl-diphosphate farnesyltransferase 1 | FDFT1 |
| 12562 | 213180_s_at | golgi SNAP receptor complex member 2 | GOSR2 |
| 11893 | 212508_at | modulator of apoptosis 1 | MOAP1 |
| 16783 | 217418_x_at | membrane-spanning 4-domains, subfamily A, member 1 | MS4A1 |
| 10423 | 210976_s_at | phosphofructokinase, muscle | PFKM |
| 4695 | 205168_at | discoidin domain receptor tyrosine kinase 2 | DDR2 |
| 1129 | 201601_x_at | interferon induced transmembrane protein 1 (9-27) | IFITM1 |
| 10109 | 210644_s_at | leukocyte-associated immunoglobulin-like receptor 1 | LAIR1 |
| 7350 | 207831_x_at | deoxyhypusine synthase | DHPS |
| 15680 | 216308_x_at | glyoxylate reductase/hydroxypyruvate reductase | GRHPR |
| 20105 | 220741_s_at | pyrophosphatase (inorganic) 2 | PPA2 |
| 13677 | 214298_x_at | septin 6 | 6-Sep |
| 1838 | 202310_s_at | collagen, type I, alpha 1 | COL1A1 |
| 7092 | 207571_x_at | chromosome 1 open reading frame 38 | C1orf38 |
| 17411 | 218046_s_at | mitochondrial ribosomal protein S16 | MRPS16 |
| 18734 | 219370_at | reprimo, TP53 dependent G2 arrest mediator candidate | RPRM |
| 3432 | 203905_at | poly(A)-specific ribonuclease (deadenylation nuclease) | PARN |
| 1376 | 201848_s_at | BCL2/adenovirus E1B 19 kDa interacting protein 3 | BNIP3 |
| 8813 | 209320_at | adenylate cyclase 3 | ADCY3 |
| 12178 | 212793_at | dishevelled associated activator of morphogenesis 2 | DAAM2 |
| 316 | 200788_s_at | phosphoprotein enriched in astrocytes 15 | PEA15 |
| 19357 | 219993_at | SRY (sex determining region Y)-box 17 | SOX17 |
| 3778 | 204251_s_at | centrosomal protein 164 kDa | CEP164 |
| 17500 | 218135_at | ERGIC and golgi 2 | ERGIC2 |
| 17890 | 218525_s_at | hypoxia-inducible factor 1, alpha subunit inhibitor | HIF1AN |
| 10976 | 211571_s_at | versican | VCAN |
| 13655 | 214276_at | Kruppel-like factor 12 | KLF12 |
| 1380 | 201852_x_at | collagen, type III, alpha 1 (Ehlers-Danlos syndrome type IV, | COL3A1 |
| | | autosomal dominant) | |
| 193 | 200665_s_at | secreted protein, acidic, cysteine-rich (osteonectin) | SPARC |
| 12801 | 213420_at | DEAH (Asp-Glu-Ala-Asp/His) box polypeptide 57 | DHX57 |
| 18564 | 219200_at | FAST kinase domains 3 | FASTKD3 |
| 1226 | 201698_s_at | splicing factor, arginine/serine-rich 9 | SFRS9 |
| 17970 | 218605_at | transcription factor B2, mitochondrial | TFB2M |
| 13247 | 213867_x_at | actin, beta | ACTB |
| 5528 | 206001_at | neuropeptide Y | NPY |
| 9733 | 210253_at | HIV-1 Tat interactive protein 2, 30 kDa | HTATIP2 |
| 4142 | 204615_x_at | isopentenyl-diphosphate delta isomerase 1 | IDI1 |
| 1483 | 201955_at | cyclin C | CCNC |
| 12276 | 212891_s_at | growth arrest and DNA-damage-inducible, gamma interacting protein 1 | GADD45GIP1 |
| 8081 | 208583_x_at | histone cluster 1, H2ai /// histone cluster 1, H2ak /// histone cluster 1, | HIST1H2AG /// HIST1H2AI /// |
| | | H2aj /// histone cluster 1, H2al /// histone cluster 1, H2am /// histone | HIST1H2AJ /// HIST1H2AK /// |
| | | cluster 1, H3f /// histone cluster 1, H2ag | HIST1H2AL /// HIST1H2AM /// |
| | | | HIST1H3F |
| 22071 | 51200_at | chromosome 19 open reading frame 60 | C19orf60 |
| 8242 | 208747_s_at | complement component 1, s subcomponent | C1S |
| 17782 | 218417_s_at | hypothetical protein FLJ20489 | FLJ20489 |
| 12535 | 213152_s_at | splicing factor, arginine/serine-rich 2B | SFRS2B |
| 2493 | 202964_s_at | regulatory factor X, 5 (influences HLA class II expression) | RFX5 |
| 12628 | 213246_at | chromosome 14 open reading frame 109 | C14orf109 |
| 12378 | 212995_x_at | family with sequence similarity 128, member B /// family with | FAM128A /// FAM128B |
| | | sequence similarity 128, member A | |
| 4983 | 205456_at | CD3e molecule, epsilon (CD3-TCR complex) | CD3E |
| 20800 | 221437_s_at | mitochondrial ribosomal protein S15 | MRPS15 |
| 17553 | 218188_s_at | translocase of inner mitochondrial membrane 13 homolog (yeast) | TIMM13 |
| 9284 | 209796_s_at | canopy 2 homolog (zebrafish) | CNPY2 |
| 3498 | 203971_at | solute carrier family 31 (copper transporters), member 1 | SLC31A1 |
| 3533 | 204006_s_at | Fc fragment of IgG, low affinity IIIa, receptor (CD16a) /// Fc | FCGR3A /// FCGR3B |
| | | fragment of IgG, low affinity IIIb, receptor (CD16b) | |
| 4611 | 205084_at | B-cell receptor-associated protein 29 | BCAP29 |
| 1618 | 202090_s_at | ubiquinol-cytochrome c reductase, 6.4 kDa subunit | UQCR |
| 22086 | 52940_at | single immunoglobulin and toll-interleukin 1 receptor (TIR) domain | SIGIRR |
| 12387 | 213004_at | angiopoietin-like 2 | ANGPTL2 |
| 3759 | 204232_at | Fc fragment of IgE, high affinity I, receptor for; gamma polypeptide | FCER1G |
| 2671 | 203143_s_at | KIAA0040 | KIAA0040 |
| 2470 | 202941_at | NADH dehydrogenase (ubiquinone) flavoprotein 2, 24 kDa | NDUFV2 |
| 19458 | 220094_s_at | coiled-coil domain containing 90A | CCDC90A |
| 8461 | 208966_x_at | interferon, gamma-inducible protein 16 | IFI16 |
| 12055 | 212670_at | elastin (supravalvular aortic stenosis, Williams-Beuren syndrome) | ELN |
| 4315 | 204788_s_at | protoporphyrinogen oxidase | PPOX |
| 3709 | 204182_s_at | zinc finger and BTB domain containing 43 | ZBTB43 |
| 3458 | 203931_s_at | mitochondrial ribosomal protein L12 | MRPL12 |
| 12370 | 212987_at | F-box protein 9 | FBXO9 |
| 4079 | 204552_at | CDNA FLJ34214 fis, clone FCBBF3021807 | — |
| 8928 | 209435_s_at | rho/rac guanine nucleotide exchange factor (GEF) 2 | ARHGEF2 |
| 10362 | 210915_x_at | T cell receptor beta constant 1 | TRBC1 |
| 14423 | 215049_x_at | CD163 molecule | CD163 |
| 15622 | 216250_s_at | leupaxin | LPXN |
| 8707 | 209213_at | carbonyl reductase 1 | CBR1 |
| 1210 | 201682_at | peptidase (mitochondrial processing) beta | PMPCB |
| 3719 | 204192_at | CD37 molecule | CD37 |
| 20674 | 221311_x_at | LYR motif containing 2 | LYRM2 |
| 2029 | 202501_at | microtubule-associated protein, RP/EB family, member 2 | MAPRE2 |
| 17085 | 217720_at | coiled-coil-helix-coiled-coil-helix domain containing 2 | CHCHD2 |
| 3051 | 203524_s_at | mercaptopyruvate sulfurtransferase | MPST |
| 2482 | 202953_at | complement component 1, q subcomponent, B chain | C1QB |
| 20963 | 221601_s_at | Fas apoptotic inhibitory molecule 3 | FAIM3 |
| 11378 | 211991_s_at | major histocompatibility complex, class II, DP alpha 1 | HLA-DPA1 |
| 18035 | 218671_s_at | ATPase inhibitory factor 1 | ATPIF1 |
| 5515 | 205988_at | CD84 molecule | CD84 |
| 4140 | 204613_at | phospholipase C, gamma 2 (phosphatidylinositol-specific) | PLCG2 |
| 18709 | 219345_at | bolA homolog 1 (E. coli) | BOLA1 |
| 8718 | 209224_s_at | NADH dehydrogenase (ubiquinone) 1 alpha subcomplex, 2, 8 kDa | NDUFA2 |
| 3765 | 204238_s_at | chromosome 6 open reading frame 108 | C6orf108 |
| 14108 | 214732_at | Sp1 transcription factor | SP1 |
| 156 | 200628_s_at | tryptophanyl-tRNA synthetase | WARS |
| 9204 | 209716_at | colony stimulating factor 1 (macrophage) | CSF1 |
| 1849 | 202321_at | geranylgeranyl diphosphate synthase 1 | GGPS1 |
| 5506 | 205979_at | secretoglobin, family 2A, member 1 | SCGB2A1 |
| 13214 | 213834_at | IQ motif and Sec7 domain 3 /// similar to IQ motif and Sec7 domain 3 | IQSEC3 /// LOC100134209 /// |
| | | /// similar to IQ motif and Sec7 domain-containing protein 3 | LOC731035 |
| 2524 | 202995_s_at | fibulin 1 | FBLN1 |
| 432 | 200904_at | major histocompatibility complex, class I, E | HLA-E |
| 21200 | 221840_at | protein tyrosine phosphatase, receptor type, E | PTPRE |
| 4420 | 204893_s_at | zinc finger, FYVE domain containing 9 | ZFYVE9 |
| 10252 | 210792_x_at | SIVA1, apoptosis-inducing factor | SIVA1 |
| 2942 | 203415_at | programmed cell death 6 | PDCD6 |
| 1871 | 202343_x_at | cytochrome c oxidase subunit Vb | COX5B |
| 4564 | 205037_at | RAB, member of RAS oncogene family-like 4 | RABL4 |
| 348 | 200820_at | proteasome (prosome, macropain) 26S subunit, non-ATPase, 8 | PSMD8 |
| 7242 | 207721_x_at | histidine triad nucleotide binding protein 1 | HINT1 |
| 14167 | 214791_at | hypothetical protein BC004921 | LOC93349 |
| 11453 | 212067_s_at | complement component 1, r subcomponent | C1R |
| 9320 | 209834_at | carbohydrate (chondroitin 6) sulfotransferase 3 | CHST3 |
| 13271 | 213892_s_at | adenine phosphoribosyltransferase | APRT |
| 21878 | 37408_at | mannose receptor, C type 2 | MRC2 |
| 4579 | 205052_at | AU RNA binding protein/enoyl-Coenzyme A hydratase | AUH |
| 19285 | 219921_s_at | dedicator of cytokinesis 5 | DOCK5 |
| 9396 | 209910_at | solute carrier family 25 (mitochondrial carrier; Graves disease | SLC25A16 |
| | | autoantigen), member 16 | |
| 2756 | 203228_at | platelet-activating factor acetylhydrolase, isoform Ib, gamma subunit | PAFAH1B3 |
| | | 29 kDa | |
| 3948 | 204421_s_at | fibroblast growth factor 2 (basic) | FGF2 |
| 2753 | 203225_s_at | riboflavin kinase | RFK |
| 19547 | 220183_s_at | nudix (nucleoside diphosphate linked moiety X)-type motif 6 | NUDT6 |
| 17338 | 217973_at | dicarbonyl/L-xylulose reductase | DCXR |
| 19297 | 219933_at | glutaredoxin 2 | GLRX2 |
| 12655 | 213274_s_at | cathepsin B | CTSB |
| 2324 | 202796_at | synaptopodin | SYNPO |
| 12353 | 212970_at | MRNA; cDNA DKFZp434E033 (from clone DKFZp434E033) | — |
| 9239 | 209751_s_at | trafficking protein particle complex 2 /// spondyloepiphyseal | SEDLP /// TRAPPC2 /// ZNF547 |
| | | dysplasia, late, pseudogene /// zinc finger protein 547 | |
| 5356 | 205829_at | hydroxysteroid (17-beta) dehydrogenase 1 | HSD17B1 |
| 21763 | 32094_at | carbohydrate (chondroitin 6) sulfotransferase 3 | CHST3 |
| 11912 | 212527_at | family with sequence similarity 152, member B | FAM152B |
| 7362 | 207843_x_at | cytochrome b5 type A (microsomal) | CYB5A |
| 2166 | 202638_s_at | intercellular adhesion molecule 1 (CD54), human rhinovirus receptor | ICAM1 |
| 18699 | 219335_at | armadillo repeat containing, X-linked 5 | ARMCX5 |
| 2214 | 202686_s_at | AXL receptor tyrosine kinase | AXL |
| 3146 | 203619_s_at | Fas apoptotic inhibitory molecule 2 | FAIM2 |
| 10156 | 210692_s_at | solute carrier family 43, member 3 | SLC43A3 |
| 13921 | 214542_x_at | histone cluster 1, H3f | HIST1H3F |
| 17200 | 217835_x_at | chromosome 20 open reading frame 24 | C20orf24 |
| 3318 | 203791_at | Dmx-like 1 | DMXL1 |
| 2313 | 202785_at | NADH dehydrogenase (ubiquinone) 1 alpha subcomplex, 7, 14.5 kDa | NDUFA7 |
| 11873 | 212488_at | collagen, type V, alpha 1 | COL5A1 |
| 8284 | 208789_at | polymerase I and transcript release factor | PTRF |
| 138 | 200610_s_at | nucleolin | NCL |
| 18915 | 219551_at | ELL associated factor 2 | EAF2 |
| 99 | 200078_s_at | ATPase, H+ transporting, lysosomal 21 kDa, V0 subunit b | ATP6V0B |
| 18869 | 219505_at | cat eye syndrome chromosome region, candidate 1 | CECR1 |
| 11466 | 212080_at | Myeloid/lymphoid or mixed-lineage leukemia (trithorax homolog, | MLL |
| | | Drosophila) | |
| 21263 | 221903_s_at | cylindromatosis (turban tumor syndrome) | CYLD |
| 19396 | 220032_at | chromosome 7 open reading frame 58 | C7orf58 |
| 577 | 201049_s_at | ribosomal protein S18 /// hypothetical protein LOC100130553 | LOC100130553 /// RPS18 |
| 17685 | 218320_s_at | NADH dehydrogenase (ubiquinone) 1 beta subcomplex, 11, 17.3 kDa | NDUFB11 |
| 958 | 201430_s_at | dihydropyrimidinase-like 3 | DPYSL3 |
| 4932 | 205405_at | sema domain, seven thrombospondin repeats (type 1 and type 1-like), | SEMA5A |
| | | transmembrane domain (TM) and short cytoplasmic domain, | |
| | | (semaphorin) 5A | |
| 17488 | 218123_at | chromosome 21 open reading frame 59 | C21orf59 |
| 19293 | 219929_s_at | zinc finger, FYVE domain containing 21 | ZFYVE21 |
| 10963 | 211558_s_at | deoxyhypusine synthase | DHPS |
| 20929 | 221566_s_at | nucleolar protein 3 (apoptosis repressor with CARD domain) | NOL3 |
| 5591 | 206065_s_at | dihydropyrimidinase | DPYS |
| 3605 | 204078_at | synaptonemal complex protein SC65 | SC65 |
| 20306 | 220942_x_at | chromosome 3 open reading frame 28 | C3orf28 |
| 21615 | 222256_s_at | hypothetical protein LOC8681 /// hypothetical protein | LOC100137047 /// |
| | | LOC100137047 | LOC100137047-PLA2G4B |
| 21151 | 221791_s_at | coiled-coil domain containing 72 | CCDC72 |
| 19362 | 219998_at | galectin-related protein | HSPC159 |
| 18747 | 219383_at | protor-2 | FLJ14213 |
| 21686 | 222327_x_at | olfactory receptor, family 7, subfamily E, member 156 pseudogene | OR7E156P |
| 18018 | 218654_s_at | mitochondrial ribosomal protein S33 | MRPS33 |
| 8577 | 209083_at | coronin, actin binding protein, 1A | CORO1A |
| 1614 | 202086_at | myxovirus (influenza virus) resistance 1, interferon-inducible protein | MX1 |
| | | p78 (mouse) | |
| 13276 | 213897_s_at | mitochondrial ribosomal protein L23 | MRPL23 |
| 1602 | 202074_s_at | optineurin | OPTN |
| 1825 | 202297_s_at | RER1 retention in endoplasmic reticulum 1 homolog (S. cerevisiae) | RER1 |
| 19961 | 220597_s_at | ADP-ribosylation-like factor 6 interacting protein 4 /// 2-oxoglutarate | ARL6IP4 /// OGFOD2 |
| | | and iron-dependent oxygenase domain containing 2 | |
| 4660 | 205133_s_at | heat shock 10 kDa protein 1 (chaperonin 10) | HSPE1 |
| 20597 | 221234_s_at | BTB and CNC homology 1, basic leucine zipper transcription factor 2 | BACH2 |
| 9980 | 210510_s_at | neuropilin 1 | NRP1 |
| 9539 | 210054_at | chromosome 4 open reading frame 15 | C4orf15 |
| 3044 | 203517_at | metaxin 2 | MTX2 |
| 642 | 201114_x_at | proteasome (prosome, macropain) subunit, alpha type, 7 | PSMA7 |
| 8436 | 208941_s_at | selenophosphate synthetase 1 | SEPHS1 |
| 663 | 201135_at | enoyl Coenzyme A hydratase, short chain, 1, mitochondrial | ECHS1 |
| 17571 | 218206_x_at | SCAN domain containing 1 | SCAND1 |
| 5031 | 205504_at | Bruton agammaglobulinemia tyrosine kinase | BTK |
| 7346 | 207827_x_at | synuclein, alpha (non A4 component of amyloid precursor) | SNCA |
| 843 | 201315_x_at | interferon induced transmembrane protein 2 (1-8D) | IFITM2 |
| 6097 | 206571_s_at | mitogen-activated protein kinase kinase kinase kinase 4 | MAP4K4 |
| 9403 | 209917_s_at | TP53 activated protein 1 | TP53AP1 |
| 3534 | 204007_at | Fc fragment of IgG, low affinity IIIb, receptor (CD16b) | FCGR3B |
| 4569 | 205042_at | glucosamine (UDP-N-acetyl)-2-epimerase/N-acetylmannosamine | GNE |
| | | kinase | |
| 11462 | 212076_at | myeloid/lymphoid or mixed-lineage leukemia (trithorax homolog, | MLL |
| | | Drosophila) | |
| 3407 | 203880_at | COX17 cytochrome c oxidase assembly homolog (S. cerevisiae) | COX17 |
| 17307 | 217942_at | mitochondrial ribosomal protein S35 | MRPS35 |
| 4672 | 205145_s_at | myosin, light chain 5, regulatory /// similar to Superfast myosin | LOC649851 /// MYL5 |
| | | regulatory light chain 2 (MyLC-2) (MYLC2) (Myosin regulatory light | |
| | | chain 5) | |
| 5313 | 205786_s_at | integrin, alpha M (complement component 3 receptor 3 subunit) | ITGAM |
| 16890 | 217525_at | olfactomedin-like 1 | OLFML1 |
| 7255 | 207734_at | lymphocyte transmembrane adaptor 1 | LAX1 |
| 18299 | 218935_at | EH-domain containing 3 | EHD3 |
| 8716 | 209222_s_at | oxysterol binding protein-like 2 | OSBPL2 |
| 12207 | 212822_at | HEG homolog 1 (zebrafish) | HEG1 |
| 2160 | 202632_at | DPH1 homolog (S. cerevisiae) /// candidate tumor suppressor in | DPH1 /// OVCA2 |
| | | ovarian cancer 2 | |
| 3409 | 203882_at | interferon regulatory factor 9 | IRF9 |
| 10111 | 210646_x_at | ribosomal protein L13a | RPL13A |
| 19017 | 219653_at | LSM14B, SCD6 homolog B (S. cerevisiae) | LSM14B |
| 15019 | 215646_s_at | versican | VCAN |
| 21485 | 222125_s_at | hypoxia-inducible factor prolyl 4-hydroxylase | PH-4 |
| 1451 | 201923_at | peroxiredoxin 4 | PRDX4 |
| 18677 | 219313_at | GRAM domain containing 1C | GRAMD1C |
| 17706 | 218341_at | phosphopantothenoylcysteine synthetase | PPCS |
| 21854 | 36830_at | mitochondrial intermediate peptidase | MIPEP |
| 11328 | 211940_x_at | H3 histone, family 3A /// H3 histone, family 3B (H3.3B) /// H3 | H3F3A /// H3F3B /// LOC440926 |
| | | histone, family 3A pseudogene | |
| 1886 | 202358_s_at | sorting nexin 19 | SNX19 |
| 2481 | 202952_s_at | ADAM metallopeptidase domain 12 (meltrin alpha) | ADAM12 |
| 6824 | 207300_s_at | coagulation factor VII (serum prothrombin conversion accelerator) | F7 |
| 21746 | 31637_s_at | thyroid hormone receptor, alpha (erythroblastic leukemia viral (v-erb- | NR1D1 /// THRA |
| | | a) oncogene homolog, avian) /// nuclear receptor subfamily 1, group | |
| | | D, member 1 | |
| 2917 | 203390_s_at | kinesin family member 3C | KIF3C |
| 13901 | 214522_x_at | histone cluster 1, H2ad /// histone cluster 1, H2bn /// histone cluster 1, | HIST1H2AD /// HIST1H2BN /// |
| | | H3a /// histone cluster 1, H3d /// histone cluster 1, H3c /// histone | HIST1H3A /// HIST1H3B /// |
| | | cluster 1, H3e /// histone cluster 1, H3i /// histone cluster 1, H3g /// | HIST1H3C /// HIST1H3D /// |
| | | histone cluster 1, H3j /// histone cluster 1, H3h /// histone cluster 1, | HIST1H3E /// HIST1H3F /// |
| | | H3b /// histone cluster 1, H3f | HIST1H3G /// HIST1H3H /// |
| | | | HIST1H3I /// HIST1H3J |
| 13113 | 213733_at | myosin IF | MYO1F |
| 12668 | 213287_s_at | keratin 10 (epidermolytic hyperkeratosis; keratosis palmaris et | KRT10 |
| | | plantaris) | |
| 20944 | 221582_at | histone cluster 3, H2a | HIST3H2A |
| 9096 | 209606_at | pleckstrin homology, Sec7 and coiled-coil domains, binding protein | PSCDBP |
| 21187 | 221827_at | RanBP-type and C3HC4-type zinc finger containing 1 | RBCK1 |
| 13051 | 213671_s_at | methionyl-tRNA synthetase | MARS |
| 21839 | 36030_at | intermediate filament family orphan | IFFO |
| 8640 | 209146_at | sterol-C4-methyl oxidase-like | SC4MOL |
| 17692 | 218327_s_at | synaptosomal-associated protein, 29 kDa | SNAP29 |
| 4678 | 205151_s_at | KIAA0644 gene product | KIAA0644 |
| 17189 | 217824_at | ubiquitin-conjugating enzyme E2, J1 (UBC6 homolog, yeast) | UBE2J1 |
| 17568 | 218203_at | asparagine-linked glycosylation 5 homolog (S. cerevisiae, dolichyl- | ALG5 |
| | | phosphate beta-glucosyltransferase) | |
| 17477 | 218112_at | mitochondrial ribosomal protein S34 | MRPS34 |
| 10354 | 210907_s_at | programmed cell death 10 | PDCD10 |
| 3440 | 203913_s_at | hydroxyprostaglandin dehydrogenase 15-(NAD) | HPGD |
| 22195 | 78383_at | similar to hCG1811779 | LOC100129250 |
| 8971 | 209478_at | stimulated by retinoic acid 13 homolog (mouse) | STRA13 |
| 18286 | 218922_s_at | LAG1 homolog, ceramide synthase 4 | LASS4 |
| 4209 | 204682_at | latent transforming growth factor beta binding protein 2 | LTBP2 |
| 17765 | 218400_at | 2′-5′-oligoadenylate synthetase 3, 100 kDa | OAS3 |
| 10374 | 210927_x_at | jumping translocation breakpoint | JTB |
| 2525 | 202996_at | polymerase (DNA-directed), delta 4 | POLD4 |
| 13653 | 214274_s_at | acetyl-Coenzyme A acyltransferase 1 (peroxisomal 3-oxoacyl- | ACAA1 |
| | | Coenzyme A thiolase) | |
| 19241 | 219877_at | zinc finger, matrin type 4 | ZMAT4 |
| 19226 | 219862_s_at | nuclear prelamin A recognition factor | NARF |
| 20640 | 221277_s_at | pseudouridylate synthase 3 | PUS3 |
| 15099 | 215726_s_at | cytochrome b5 type A (microsomal) | CYB5A |
| 4691 | 205164_at | glycine C-acetyltransferase (2-amino-3-ketobutyrate coenzyme A | GCAT |
| | | ligase) | |
| 8376 | 208881_x_at | isopentenyl-diphosphate delta isomerase 1 | IDI1 |
| 9365 | 209879_at | selectin P ligand | SELPLG |
| 11619 | 212233_at | microtubule-associated protein 1B | MAP1B |
| 3016 | 203489_at | SIVA1, apoptosis-inducing factor | SIVA1 |
| 18647 | 219283_at | C1GALT1-specific chaperone 1 | C1GALT1C1 |
| 21053 | 221692_s_at | mitochondrial ribosomal protein L34 | MRPL34 |
| 1707 | 202179_at | bleomycin hydrolase | BLMH |
| 11732 | 212347_x_at | MAX dimerization protein 4 | MXD4 |
| 11576 | 212190_at | serpin peptidase inhibitor, clade E (nexin, plasminogen activator | SERPINE2 |
| | | inhibitor type 1), member 2 | |
| 17466 | 218101_s_at | NADH dehydrogenase (ubiquinone) 1, subcomplex unknown, 2, | NDUFC2 |
| | | 14.5 kDa | |
| 11577 | 212191_x_at | ribosomal protein L13 | RPL13 |
| 9435 | 209949_at | neutrophil cytosolic factor 2 (65 kDa, chronic granulomatous disease, | NCF2 |
| | | autosomal 2) | |
| 8806 | 209312_x_at | major histocompatibility complex, class II, DQ beta 1 /// major | hCG_1998957 /// HLA-DQB1 /// |
| | | histocompatibility complex, class II, DQ beta 2 /// major | HLA-DQB2 /// HLA-DRB1 /// |
| | | histocompatibility complex, class II, DR beta 1 /// major | HLA-DRB2 /// HLA-DRB3 /// |
| | | histocompatibility complex, class II, DR beta 2 (pseudogene) /// | HLA-DRB4 /// HLA-DRB5 /// |
| | | major histocompatibility complex, class II, DR beta 3 /// major | LOC100133484 /// |
| | | histocompatibility complex, class II, DR beta 4 /// major | LOC100133583 /// |
| | | histocompatibility complex, class II, DR beta 5 /// ribonuclease, | LOC100133661 /// |
| | | RNase A family, 2 (liver, eosinophil-derived neurotoxin) /// zinc | LOC100133811 /// LOC730415 /// |
| | | finger protein 749 /// hypothetical protein LOC730415 /// similar to | RNASE2 /// ZNF749 |
| | | Major histocompatibility complex, class II, DR beta 4 /// similar to | |
| | | major histocompatibility complex, class II, DQ beta 1 /// similar to | |
| | | HLA class II histocompatibility antigen, DR-W53 beta chain /// | |
| | | similar to hCG1992647 | |
| 12466 | 213083_at | solute carrier family 35, member D2 | SLC35D2 |
| 3351 | 203824_at | tetraspanin 8 | TSPAN8 |
| 13603 | 214224_s_at | protein (peptidylprolyl cis/trans isomerase) NIMA-interacting, 4 | PIN4 |
| | | (parvulin) | |
| 6874 | 207351_s_at | SH2 domain protein 2A | SH2D2A |
| 17896 | 218531_at | transmembrane protein 134 | TMEM134 |
| 1421 | 201893_x_at | decorin | DCN |
| 21204 | 221844_x_at | CDNA clone IMAGE: 6208446 | — |
| 4012 | 204485_s_at | target of myb1 (chicken)-like 1 | TOM1L1 |
| 241 | 200713_s_at | microtubule-associated protein, RP/EB family, member 1 | MAPRE1 |
| 3561 | 204034_at | ethylmalonic encephalopathy 1 | ETHE1 |
| 10458 | 211012_s_at | promyelocytic leukemia /// hypothetical protein LOC161527 | LOC161527 /// PML |
| 11192 | 211796_s_at | T cell receptor beta constant 1 | TRBC1 |
| 10471 | 211025_x_at | cytochrome c oxidase subunit Vb | COX5B |
| 13519 | 214140_at | solute carrier family 25 (mitochondrial carrier; Graves disease | SLC25A16 |
| | | autoantigen), member 16 | |
| 4395 | 204868_at | immature colon carcinoma transcript 1 | ICT1 |
| 5278 | 205751_at | SH3-domain GRB2-like 2 | SH3GL2 |
| 7212 | 207691_x_at | ectonucleoside triphosphate diphosphohydrolase 1 | ENTPD1 |
| 3969 | 204442_x_at | latent transforming growth factor beta binding protein 4 | LTBP4 |
| 11486 | 212100_s_at | polymerase (DNA-directed), delta interacting protein 3 | POLDIP3 |
| 607 | 201079_at | synaptogyrin 2 | SYNGR2 |
| 15854 | 216483_s_at | chromosome 19 open reading frame 10 | C19orf10 |
| 18483 | 219119_at | LSM8 homolog, U6 small nuclear RNA associated (S. cerevisiae) | LSM8 |
| 4132 | 204605_at | cell growth regulator with ring finger domain 1 | CGRRF1 |
| 4686 | 205159_at | colony stimulating factor 2 receptor, beta, low-affinity (granulocyte- | CSF2RB |
| | | macrophage) | |
| 4874 | 205347_s_at | thymosin-like 8 /// thymosin beta15b | MGC39900 /// TMSL8 |
| 11632 | 212246_at | multiple coagulation factor deficiency 2 | MCFD2 |
| 18881 | 219517_at | elongation factor RNA polymerase II-like 3 | ELL3 |
| 9285 | 209797_at | canopy 2 homolog (zebrafish) | CNPY2 |
| 17263 | 217898_at | chromosome 15 open reading frame 24 | C15orf24 |
| 3362 | 203835_at | leucine rich repeat containing 32 | LRRC32 |
| 20972 | 221610_s_at | signal transducing adaptor family member 2 | STAP2 |
| 1315 | 201787_at | fibulin 1 /// similar to Fibulin 1 | FBLN1 /// LOC100133843 |
| 12031 | 212646_at | raftlin, lipid raft linker 1 | RFTN1 |
| 8995 | 209502_s_at | BAI1-associated protein 2 | BAIAP2 |
| 2385 | 202857_at | canopy 2 homolog (zebrafish) | CNPY2 |
| 18145 | 218781_at | structural maintenance of chromosomes 6 | SMC6 |
| 3143 | 203616_at | polymerase (DNA directed), beta | POLB |
| 21790 | 336_at | thromboxane A2 receptor | TBXA2R |
| 533 | 201005_at | CD9 molecule | CD9 |
| 17236 | 217871_s_at | macrophage migration inhibitory factor (glycosylation-inhibiting | MIF |
| | | factor) | |
| 12631 | 213249_at | F-box and leucine-rich repeat protein 7 | FBXL7 |
| 21186 | 221826_at | angel homolog 2 (Drosophila) | ANGEL2 |
| 502 | 200974_at | actin, alpha 2, smooth muscle, aorta | ACTA2 |
| 17277 | 217912_at | dihydrouridine synthase 1-like (S. cerevisiae) | DUS1L |
| 4348 | 204821_at | butyrophilin, subfamily 3, member A3 | BTN3A3 |
| 6549 | 207023_x_at | keratin 10 (epidermolytic hyperkeratosis; keratosis palmaris et | KRT10 |
| | | plantaris) | |
| 8437 | 208942_s_at | SEC62 homolog (S. cerevisiae) | SEC62 |
| 10502 | 211058_x_at | tubulin, alpha 1b | TUBA1B |
| 2499 | 202970_at | dual-specificity tyrosine-(Y)-phosphorylation regulated kinase 2 | DYRK2 |
| 8424 | 208929_x_at | ribosomal protein L13 | RPL13 |
| 18333 | 218969_at | mitochondria-associated protein involved in granulocyte-macrophage | Magmas |
| | | colony-stimulating factor signal transduction | |
| 4336 | 204809_at | ClpX caseinolytic peptidase X homolog (E. coli) | CLPX |
| 3843 | 204316_at | regulator of G-protein signaling 10 | RGS10 |
| 19859 | 220495_s_at | thioredoxin domain containing 15 | TXNDC15 |
| 17644 | 218279_s_at | histone cluster 2, H2aa3 | HIST2H2AA3 |
| 12581 | 213199_at | C2 calcium-dependent domain containing 3 | C2CD3 |
| 2268 | 202740_at | aminoacylase 1 | ACY1 |
| 12671 | 213290_at | collagen, type VI, alpha 2 | COL6A2 |
| 3381 | 203854_at | complement factor I | CFI |
| 17662 | 218297_at | chromosome 10 open reading frame 97 | C10orf97 |
| 19698 | 220334_at | regulator of G-protein signaling 17 | RGS17 |
| 13343 | 213964_x_at | CDNA FLJ37852 fis, clone BRSSN2014513 | — |
| 3919 | 204392_at | calcium/calmodulin-dependent protein kinase I | CAMK1 |
| 15667 | 216295_s_at | clathrin, light chain (Lca) | CLTA |
| 3174 | 203647_s_at | ferredoxin 1 | FDX1 |
| 13267 | 213888_s_at | TRAF3 interacting protein 3 /// hypothetical protein LOC100133233 | LOC100133233 /// TRAF3IP3 |
| 18230 | 218866_s_at | polymerase (RNA) III (DNA directed) polypeptide K, 12.3 kDa | POLR3K |
| 18379 | 219015_s_at | asparagine-linked glycosylation 13 homolog (S. cerevisiae) /// | ALG13 /// CXorf45 |
| | | chromosome X open reading frame 45 | |
| 4092 | 204565_at | thioesterase superfamily member 2 | THEM2 |
| 8332 | 208837_at | transmembrane emp24 protein transport domain containing 3 | TMED3 |
| 6644 | 207118_s_at | matrix metallopeptidase 23B /// matrix metallopeptidase 23A | MMP23A /// MMP23B |
| | | (pseudogene) | |
| 7131 | 207610_s_at | egf-like module containing, mucin-like, hormone receptor-like 2 | EMR2 |
| 21448 | 222088_s_at | solute carrier family 2 (facilitated glucose transporter), member 3 /// | SLC2A14 /// SLC2A3 |
| | | solute carrier family 2 (facilitated glucose transporter), member 14 | |
| 2106 | 202578_s_at | DEAD (Asp-Glu-Ala-As) box polypeptide 19A | DDX19A |
| 11917 | 212532_s_at | LSM12 homolog (S. cerevisiae) | LSM12 |
| 9279 | 209791_at | peptidyl arginine deiminase, type II | PADI2 |
| 2680 | 203152_at | mitochondrial ribosomal protein L40 | MRPL40 |
| 9556 | 210072_at | chemokine (C—C motif) ligand 19 | CCL19 |
| 3725 | 204198_s_at | runt-related transcription factor 3 | RUNX3 |
| 6059 | 206533_at | cholinergic receptor, nicotinic, alpha 5 | CHRNA5 |
| 886 | 201358_s_at | coatomer protein complex, subunit beta 1 | COPB1 |
| 9222 | 209734_at | NCK-associated protein 1-like | NCKAP1L |
| 3074 | 203547_at | CD4 molecule | CD4 |
| 11589 | 212203_x_at | interferon induced transmembrane protein 3 (1-8U) | IFITM3 |
| 4866 | 205339_at | SCL/TAL1 interrupting locus | STIL |
| 20450 | 221087_s_at | apolipoprotein L, 3 | APOL3 |
| 12424 | 213041_s_at | ATP synthase, H+ transporting, mitochondrial F1 complex, delta | ATP5D |
| | | subunit | |
| 13711 | 214332_s_at | Ts translation elongation factor, mitochondrial | TSFM |
| 9369 | 209883_at | glycosyltransferase 25 domain containing 2 | GLT25D2 |
| 1128 | 201600_at | prohibitin 2 | PHB2 |
| 1484 | 201956_s_at | glyceronephosphate O-acyltransferase | GNPAT |
| 215 | 200687_s_at | splicing factor 3b, subunit 3, 130 kDa | SF3B3 |
| 10831 | 211421_s_at | ret proto-oncogene | RET |
| 3449 | 203922_s_at | cytochrome b-245, beta polypeptide (chronic granulomatous disease) | CYBB |
| 2943 | 203416_at | CD53 molecule | CD53 |
| 5126 | 205599_at | TNF receptor-associated factor 1 | TRAF1 |
| 19082 | 219718_at | FGGY carbohydrate kinase domain containing | FGGY |
| 15935 | 216565_x_at | — | — |
| 11115 | 211714_x_at | tubulin, beta | TUBB |
| 9299 | 209813_x_at | TCR gamma alternate reading frame protein | TARP |
| 18452 | 219088_s_at | zinc finger protein 576 | ZNF576 |
| 9072 | 209582_s_at | CD200 molecule | CD200 |
| 65 | 200044_at | splicing factor, arginine/serine-rich 9 | SFRS9 |
| 9315 | 209829_at | chromosome 6 open reading frame 32 | C6orf32 |
| 3791 | 204264_at | carnitine palmitoyltransferase II | CPT2 |
| 19566 | 220202_s_at | ring finger and CCCH-type zinc finger domains 2 | RC3H2 |
| 5296 | 205769_at | solute carrier family 27 (fatty acid transporter), member 2 | SLC27A2 |
| 2165 | 202637_s_at | intercellular adhesion molecule 1 (CD54), human rhinovirus receptor | ICAM1 |
| 4147 | 204620_s_at | versican | VCAN |
| 3193 | 203666_at | chemokine (C—X—C motif) ligand 12 (stromal cell-derived factor 1) | CXCL12 |
| 5187 | 205660_at | 2′-5′-oligoadenylate synthetase-like | OASL |
| 7937 | 208438_s_at | Gardner-Rasheed feline sarcoma viral (v-fgr) oncogene homolog | FGR |
| 17633 | 218268_at | TBC1 domain family, member 15 | TBC1D15 |
| 11307 | 211919_s_at | chemokine (C—X—C motif) receptor 4 | CXCR4 |
| 14338 | 214963_at | nucleoporin 160 kDa | NUP160 |
| 9032 | 209539_at | Rac/Cdc42 guanine nucleotide exchange factor (GEF) 6 | ARHGEF6 |
| 6860 | 207336_at | SRY (sex determining region Y)-box 5 | SOX5 |
| 4764 | 205237_at | ficolin (collagen/fibrinogen domain containing) 1 | FCN1 |
| 13842 | 214463_x_at | histone cluster 1, H4j | HIST1H4J |
| 18481 | 219117_s_at | FK506 binding protein 11, 19 kDa | FKBP11 |
| 11641 | 212255_s_at | ATPase, Ca++ transporting, type 2C, member 1 | ATP2C1 |
| 675 | 201147_s_at | TIMP metallopeptidase inhibitor 3 (Sorsby fundus dystrophy, | TIMP3 |
| | | pseudoinflammatory) | |
| 7916 | 208415_x_at | inhibitor of growth family, member 1 | ING1 |
| 3521 | 203994_s_at | chromosome 21 open reading frame 2 | C21orf2 |
| 10246 | 210786_s_at | Friend leukemia virus integration 1 | FLI1 |
| 17805 | 218440_at | methylcrotonoyl-Coenzyme A carboxylase 1 (alpha) | MCCC1 |
| 13737 | 214358_at | acetyl-Coenzyme A carboxylase alpha | ACACA |
| 18440 | 219076_s_at | peroxisomal membrane protein 2, 22 kDa | PXMP2 |
| 9277 | 209789_at | coronin, actin binding protein, 2B | CORO2B |
| 19509 | 220145_at | microtubule-associated protein 9 | MAP9 |
| 2752 | 203224_at | riboflavin kinase | RFK |
| 19335 | 219971_at | interleukin 21 receptor | IL21R |
| 13379 | 214000_s_at | Regulator of G-protein signaling 10 | RGS10 |
| 2843 | 203316_s_at | small nuclear ribonucleoprotein polypeptide E | SNRPE |
| 959 | 201431_s_at | dihydropyrimidinase-like 3 | DPYSL3 |
| 1219 | 201691_s_at | tumor protein D52 | TPD52 |
| 12131 | 212746_s_at | centrosomal protein 170 kDa | CEP170 |
| 1837 | 202309_at | methylenetetrahydrofolate dehydrogenase (NADP+ dependent) 1, | MTHFD1 |
| | | methenyltetrahydrofolate cyclohydrolase, formyltetrahydrofolate | |
| | | synthetase | |
| 3289 | 203762_s_at | dynein, cytoplasmic 2, light intermediate chain 1 | DYNC2LI1 |
| 1696 | 202168_at | TAF9 RNA polymerase II, TATA box binding protein (TBP)- | TAF9 |
| | | associated factor, 32 kDa | |
| 2367 | 202839_s_at | NADH dehydrogenase (ubiquinone) 1 beta subcomplex, 7, 18 kDa | NDUFB7 |
| 634 | 201106_at | glutathione peroxidase 4 (phospholipid hydroperoxidase) | GPX4 |
| 18457 | 219093_at | phosphotyrosine interaction domain containing 1 | PID1 |
| 19064 | 219700_at | plexin domain containing 1 | PLXDC1 |
| 4512 | 204985_s_at | trafficking protein particle complex 6A | TRAPPC6A |
| 13631 | 214252_s_at | ceroid-lipofuscinosis, neuronal 5 | CLN5 |
| 20380 | 221016_s_at | transcription factor 7-like 1 (T-cell specific, HMG-box) | TCF7L1 |
| 3050 | 203523_at | lymphocyte-specific protein 1 | LSP1 |
| 1666 | 202138_x_at | JTV1 gene | JTV1 |
| 2915 | 203388_at | arrestin, beta 2 | ARRB2 |
| 1191 | 201663_s_at | structural maintenance of chromosomes 4 | SMC4 |
| 2425 | 202897_at | signal-regulatory protein alpha | SIRPA |
| 11834 | 212449_s_at | lysophospholipase I | LYPLA1 |
| 14070 | 214694_at | myosin phosphatase-Rho interacting protein /// similar to Myosin | LOC729143 /// M-RIP |
| | | phosphatase Rho-interacting protein (Rho-interacting protein 3) (M- | |
| | | RIP) (RIP3) (p116Rip) | |
| 10128 | 210663_s_at | kynureninase (L-kynurenine hydrolase) | KYNU |
| 17957 | 218592_s_at | cat eye syndrome chromosome region, candidate 5 | CECR5 |
| 2747 | 203219_s_at | adenine phosphoribosyltransferase | APRT |
| 4923 | 205396_at | SMAD family member 3 | SMAD3 |
| 13528 | 214149_s_at | ATPase, H+ transporting, lysosomal 9 kDa, V0 subunit e1 | ATP6V0E1 |
| 9209 | 209721_s_at | intermediate filament family orphan | IFFO |
| 1708 | 202180_s_at | major vault protein | MVP |
| 11871 | 212486_s_at | FYN oncogene related to SRC, FGR, YES | FYN |
| 10719 | 211296_x_at | ribosomal protein S27a /// ubiquitin B /// ubiquitin C | RPS27A /// UBB /// UBC |
| 2625 | 203096_s_at | Rap guanine nucleotide exchange factor (GEF) 2 | RAPGEF2 |
| 21046 | 221685_s_at | coiled-coil domain containing 99 | CCDC99 |
| 9080 | 209590_at | bone morphogenetic protein 7 (osteogenic protein 1) | BMP7 |
| 17132 | 217767_at | complement component 3 | C3 |
| 16391 | 217021_at | cytochrome b5 type A (microsomal) | CYB5A |
| 12705 | 213324_at | v-src sarcoma (Schmidt-Ruppin A-2) viral oncogene homolog (avian) | SRC |
| 4937 | 205410_s_at | ATPase, Ca++ transporting, plasma membrane 4 | ATP2B4 |
| 4005 | 204478_s_at | RAB interacting factor | RABIF |
| 2450 | 202921_s_at | ankyrin 2, neuronal | ANK2 |
| 17587 | 218222_x_at | aryl hydrocarbon receptor nuclear translocator | ARNT |
| 11739 | 212354_at | sulfatase 1 | SULF1 |
| 17563 | 218198_at | DEAH (Asp-Glu-Ala-His) box polypeptide 32 | DHX32 |
| 2998 | 203471_s_at | pleckstrin | PLEK |
| 817 | 201289_at | cysteine-rich, angiogenic inducer, 61 | CYR61 |
| 13208 | 213828_x_at | H3 histone, family 3A /// H3 histone, family 3B (H3.3B) /// H3 | H3F3A /// H3F3B /// LOC440926 |
| | | histone, family 3A pseudogene | |
| 2643 | 203114_at | Sjogren syndrome/scleroderma autoantigen 1 | SSSCA1 |
| 11155 | 211755_s_at | ATP synthase, H+ transporting, mitochondrial F0 complex, subunit | ATP5F1 |
| | | B1 | |
| 313 | 200785_s_at | low density lipoprotein-related protein 1 (alpha-2-macroglobulin | LOC100134190 /// LRP1 |
| | | receptor) /// similar to low density lipoprotein-related protein 1 | |
| | | (alpha-2-macroglobulin receptor) | |
| 3107 | 203580_s_at | solute carrier family 7 (cationic amino acid transporter, y+ system), | SLC7A6 /// TRPV6 |
| | | member 6 /// transient receptor potential cation channel, subfamily V, | |
| | | member 6 | |
| 1797 | 202269_x_at | guanylate binding protein 1, interferon-inducible, 67 kDa | GBP1 |
| 6616 | 207090_x_at | zinc finger protein 30 homolog (mouse) | ZFP30 |
| 22150 | 61734_at | reticulocalbin 3, EF-hand calcium binding domain | RCN3 |
| 1605 | 202077_at | NADH dehydrogenase (ubiquinone) 1, alpha/beta subcomplex, 1, | NDUFAB1 |
| | | 8 kDa | |
| 9392 | 209906_at | complement component 3a receptor 1 | C3AR1 |
| 11125 | 211725_s_at | BH3 interacting domain death agonist | BID |
| 22063 | 50374_at | chromosome 17 open reading frame 90 | C17orf90 |
| 11116 | 211715_s_at | 3-hydroxybutyrate dehydrogenase, type 1 | BDH1 |
| 6371 | 206845_s_at | ring finger protein 40 | RNF40 |
| 3047 | 203520_s_at | zinc finger protein 318 | ZNF318 |
| 2069 | 202541_at | small inducible cytokine subfamily E, member 1 (endothelial | SCYE1 |
| | | monocyte-activating) | |
| 11842 | 212457_at | transcription factor binding to IGHM enhancer 3 | TFE3 |
| 22172 | 64942_at | G protein-coupled receptor 153 | GPR153 |
| 4297 | 204770_at | transporter 2, ATP-binding cassette, sub-family B (MDR/TAP) | TAP2 |
| 3406 | 203879_at | phosphoinositide-3-kinase, catalytic, delta polypeptide | PIK3CD |
| 10098 | 210633_x_at | keratin 10 (epidermolytic hyperkeratosis; keratosis palmaris et | KRT10 |
| | | plantaris) | |
| 8568 | 209074_s_at | family with sequence similarity 107, member A | FAM107A |
| 8970 | 209477_at | emerin (Emery-Dreifuss muscular dystrophy) | EMD |
| 12512 | 213129_s_at | glycine cleavage system protein H (aminomethyl carrier) /// similar to | GCSH /// LOC730107 |
| | | Glycine cleavage system H protein, mitochondrial | |
| 14534 | 215160_x_at | similar to FRG1 protein (FSHD region gene 1 protein) | LOC642236 |
| 14490 | 215116_s_at | dynamin 1 | DNM1 |
| 4994 | 205467_at | caspase 10, apoptosis-related cysteine peptidase | CASP10 |
| 8941 | 209448_at | HIV-1 Tat interactive protein 2, 30 kDa | HTATIP2 |
| 10061 | 210596_at | magnesium transporter 1 /// similar to PRO0756 | LOC100129513 /// |
| | | | LOC100133276 /// MAGT1 |
| 3441 | 203914_x_at | hydroxyprostaglandin dehydrogenase 15-(NAD) | HPGD |

The multiple linear regression method was extended to divide tumor cases into those with good outcome (never relapsed following surgery, i.e. appear to be cured) from bad outcome, i.e. in several months or years following surgery their tumor reappeared. The genes that are specifically differentially expressed in the bad outcome cases were identified (the list). These genes or a subset of them may be measure in a new patient to determine whether he matches a good or bad outcome profile. In summary, differences in RNA levels that correlated with relapse versus non-relapse were calculated for four expression microarray data sets (data set 1, 2, 3 and 4) using multiple linear regression models which used these percentages in a linear model. Many of these relapse-associated changes in transcript levels occurred in adjacent stroma. Data set 3 does not have pathologist's estimation of tissue percentage and in silico tissue prediction model was used to predict tissue percentages. The identified genes are listed in Tables 35-42.

TABLE 35
dataset 1 (stroma)

| | | | log base 2 | |
|---|---|---|---|---|
| uniqueID | GeneSymb | GeneDesc | fold change | probS |
| 203381_s_at | APOE | apolipoprotein E | 2.912890091 | 4.87E−05 |
| 201465_s_at | JUN | v-jun sarcoma virus 17 oncogene homolog (avian) | 2.770294931 | 0.005679 |
| 221011_s_at | LBH | likely ortholog of mouse limb-bud and heart gene /// likely ortholog of | 2.758949055 | 0.008186 |
| | | mouse limb-bud and heart gene | | |
| 203382_s_at | APOE | apolipoprotein E | 2.683386924 | 2.64E−06 |
| 201428_at | CLDN4 | claudin 4 | 2.681309084 | 4.34E−05 |
| 203953_s_at | CLDN3 | claudin 3 | 2.547551209 | 0.009406 |
| 203400_s_at | TF | transferrin | 2.544986614 | 0.000238 |
| 200923_at | LGALS3BP | lectin, galactoside-binding, soluble, 3 binding protein | 2.278659056 | 3.32E−05 |
| 212016_s_at | PTBP1 | polypyrimidine tract binding protein 1 | 2.119201234 | 0.004097 |
| 213986_s_at | C19orf6 | chromosome 19 open reading frame 6 | 2.088646742 | 0.005784 |
| 203954_x_at | CLDN3 | claudin 3 | 2.084660768 | 0.001916 |
| 219127_at | MGC11242 | hypothetical protein MGC11242 | 2.072553416 | 0.002204 |
| 201670_s_at | MARCKS | myristoylated alanine-rich protein kinase C substrate | 2.071304593 | 0.003602 |
| 200796_s_at | MCL1 | myeloid cell leukemia sequence 1 (BCL2-related) | 2.065871088 | 0.000335 |
| 211919_s_at | CXCR4 | chemokine (C—X—C motif) receptor 4 /// chemokine (C—X—C motif) | 2.007551333 | 0.005134 |
| | | receptor 4 | | |
| 208960_s_at | KLF6 | Kruppel-like factor 6 | 1.98241705 | 0.001315 |
| 203878_s_at | MMP11 | matrix metallopeptidase 11 (stromelysin 3) | 1.946981553 | 0.000644 |
| 208961_s_at | KLF6 | Kruppel-like factor 6 | 1.928324021 | 0.001944 |
| 201418_s_at | SOX4 | SRY (sex determining region Y)-box 4 | 1.886396014 | 0.00012 |
| 209201_x_at | CXCR4 | chemokine (C—X—C motif) receptor 4 | 1.875714407 | 0.004034 |
| 218010_x_at | C20orf149 | chromosome 20 open reading frame 149 | 1.875322213 | 0.001173 |
| 201169_s_at | BHLHB2 | basic helix-loop-helix domain containing, class B, 2 | 1.860022182 | 0.001173 |
| 211796_s_at | TRBV21-1 /// | T cell receptor beta variable 21-1 /// T cell receptor beta variable 19 /// T | 1.852987215 | 0.006799 |
| | TRBV19 /// | cell receptor beta variable 5-4 /// T cell receptor beta variable 3-1 /// T | | |
| | TRBV5-4 /// | cell receptor beta constant 1 | | |
| | TRBV3-1 /// | | | |
| | TRBC1 | | | |
| 210827_s_at | ELF3 | E74-like factor 3 (ets domain transcription factor, epithelial-specific) | 1.852964164 | 0.00592 |
| 201235_s_at | BTG2 | BTG family, member 2 | 1.834631974 | 0.005075 |
| 204103_at | CCL4 | chemokine (C—C motif) ligand 4 | 1.830777096 | 0.002071 |
| 211656_x_at | HLA-DQB1 | major histocompatibility complex, class II, DQ beta 1 /// major | 1.826633903 | 0.000461 |
| | | histocompatibility complex, class II, DQ beta 1 | | |
| 217478_s_at | HLA-DMA | major histocompatibility complex, class II, DM alpha | 1.804916186 | 0.002235 |
| 211991_s_at | HLA-DPA1 | major histocompatibility complex, class II, DP alpha 1 | 1.778213183 | 0.009104 |
| 201467_s_at | NQO1 | NAD(P)H dehydrogenase, quinone 1 | 1.724578298 | 0.008336 |
| 214063_s_at | TF | transferrin | 1.719846565 | 5.39E−06 |
| 204151_x_at | AKR1C1 | aldo-keto reductase family 1, member C1 (dihydrodiol dehydrogenase 1; | 1.703218877 | 0.005958 |
| | | 20-alpha (3-alpha)-hydroxysteroid dehydrogenase) | | |
| 213002_at | MARCKS | Myristoylated alanine-rich protein kinase C substrate | 1.695018082 | 0.003443 |
| 212884_x_at | APOE | Apolipoprotein E | 1.690228647 | 3.37E−05 |
| 210130_s_at | TM7SF2 | transmembrane 7 superfamily member 2 | 1.636804642 | 0.0033 |
| 203876_s_at | MMP11 | matrix metallopeptidase 11 (stromelysin 3) | 1.633132547 | 0.004348 |
| 221267_s_at | C19orf27 | chromosome 19 open reading frame 27 /// chromosome 19 open | 1.591104024 | 0.000266 |
| | | reading frame 27 | | |
| 203206_at | FAM53B | family with sequence similarity 53, member B | 1.546295766 | 0.009117 |
| 202803_s_at | ITGB2 | integrin, beta 2 (antigen CD18 (p95), lymphocyte function-associated | 1.525053858 | 0.001955 |
| | | antigen 1; macrophage antigen 1 (mac-1) beta subunit) | | |
| 204805_s_at | H1FX | H1 histone family, member X | 1.51177251 | 0.000833 |
| 203085_s_at | TGFB1 | transforming growth factor, beta 1 (Camurati-Engelmann disease) | 1.496141641 | 0.006601 |
| 201102_s_at | PFKL | phosphofructokinase, liver | 1.494139392 | 0.003596 |
| 222037_at | MCM4 | MCM4 minichromosome maintenance deficient 4 (S. cerevisiae) | 1.492902786 | 0.008226 |
| 213603_s_at | RAC2 | ras-related C3 botulinum toxin substrate 2 (rho family, small GTP | 1.421863261 | 0.001516 |
| | | binding protein Rac2) | | |
| 211065_x_at | PFKL | phosphofructokinase, liver /// phosphofructokinase, liver | 1.419074476 | 0.003626 |
| 209872_s_at | PKP3 | plakophilin 3 | 1.41708755 | 0.003264 |
| 218500_at | C8orf55 | chromosome 8 open reading frame 55 | 1.40325073 | 0.000829 |
| 222001_x_at | — | LOC440669 | 1.381519268 | 0.001264 |
| 201721_s_at | LAPTM5 | lysosomal associated multispanning membrane protein 5 | 1.364245523 | 0.004506 |
| 204480_s_at | C9orf16 | chromosome 9 open reading frame 16 | 1.362119551 | 0.002395 |
| 202424_at | MAP2K2 | mitogen-activated protein kinase kinase 2 | 1.348325659 | 0.008211 |
| 218400_at | OAS3 | 2′-5′-oligoadenylate synthetase 3, 100 kDa | 1.347681406 | 0.006973 |
| 218388_at | PGLS | 6-phosphogluconolactonase | 1.331769877 | 0.003923 |
| 1405_i_at | CCL5 | chemokine (C—C motif) ligand 5 | 1.330972453 | 0.006929 |
| 209083_at | CORO1A | coronin, actin binding protein, 1A | 1.330478184 | 0.00188 |
| 217580_x_at | ARL6IP2 | ADP-ribosylation factor-like 6 interacting protein 2 | 1.326808911 | 0.000724 |
| 212575_at | C19orf6 | chromosome 19 open reading frame 6 | 1.322896709 | 0.008636 |
| 201137_s_at | HLA-DPB1 | major histocompatibility complex, class II, DP beta 1 | 1.317915963 | 0.000445 |
| 222329_x_at | ANKRD17 | Ankyrin repeat domain 17 | 1.305908564 | 0.003892 |
| 213756_s_at | HSF1 | heat shock transcription factor 1 | 1.28322941 | 0.001029 |
| 211528_x_at | HLA-G | HLA-G histocompatibility antigen, class I, G | 1.254908775 | 0.007445 |
| 208306_x_at | HLA-DRB5 | Major histocompatibility complex, class II, DR beta 3 | 1.252929469 | 0.009424 |
| 203669_s_at | DGAT1 | diacylglycerol O-acyltransferase homolog 1 (mouse) | 1.251384492 | 0.002648 |
| 209034_at | PNRC1 | proline-rich nuclear receptor coactivator 1 | 1.22881177 | 0.00706 |
| 218387_s_at | PGLS | 6-phosphogluconolactonase | 1.218121181 | 0.000973 |
| 216641_s_at | LAD1 | ladinin 1 | 1.216435002 | 0.001758 |
| 41047_at | C9orf16 | chromosome 9 open reading frame 16 | 1.216180961 | 0.00049 |
| 215256_x_at | SNX26 | sorting nexin 26 | 1.214981235 | 0.007326 |
| 203655_at | XRCC1 | X-ray repair complementing defective repair in Chinese hamster cells 1 | 1.208264842 | 5.44E−06 |
| 216905_s_at | ST14 | suppression of tumorigenicity 14 (colon carcinoma, matriptase, epithin) | 1.204813841 | 5.15E−05 |
| 219709_x_at | C16orf24 | chromosome 16 open reading frame 24 | 1.201397295 | 0.002638 |
| 216237_s_at | MCM5 | MCM5 minichromosome maintenance deficient 5, cell division cycle 46 | 1.196420347 | 0.00013 |
| | | (S. cerevisiae) | | |
| 214630_at | CYP11B2 | cytochrome P450, family 11, subfamily B, polypeptide 2 | 1.192597779 | 0.00666 |
| 216180_s_at | SYNJ2 | synaptojanin 2 | 1.181448547 | 0.003155 |
| 219986_s_at | ACAD10 | acyl-Coenzyme A dehydrogenase family, member 10 | 1.170864828 | 0.008871 |
| 209617_s_at | CTNND2 | catenin (cadherin-associated protein), delta 2 (neural plakophilin-related | 1.151479966 | 0.008055 |
| | | arm-repeat protein) | | |
| 205462_s_at | HPCAL1 | hippocalcin-like 1 | 1.147720101 | 0.004591 |
| 221410_x_at | PCDHB3 | protocadherin beta 3 | 1.130409165 | 0.007588 |
| 216526_x_at | HLA-C | major histocompatibility complex, class I, C | 1.129954807 | 0.002574 |
| 202574_s_at | CSNK1G2 | casein kinase 1, gamma 2 | 1.127593225 | 0.003117 |
| 210974_s_at | AP3D1 | adaptor-related protein complex 3, delta 1 subunit | 1.121470759 | 0.007399 |
| 216603_at | SLC7A8 | Solute carrier family 7 (cationic amino acid transporter, y+ system), | 1.115393112 | 0.002945 |
| | | member 8 | | |
| 218414_s_at | NDE1 | nudE nuclear distribution gene E homolog 1 (A. nidulans) | 1.109296453 | 0.005488 |
| 201750_s_at | ECE1 | endothelin converting enzyme 1 | 1.105981324 | 0.001328 |
| 213072_at | CYHR1 | cysteine/histidine-rich 1 | 1.098248049 | 0.001836 |
| 202005_at | ST14 | suppression of tumorigenicity 14 (colon carcinoma, matriptase, epithin) | 1.075171037 | 4.02E−05 |
| 217456_x_at | HLA-E | major histocompatibility complex, class I, E | 1.074487488 | 0.009378 |
| 219189_at | FBXL6 | F-box and leucine-rich repeat protein 6 | 1.069653172 | 0.002043 |
| 209969_s_at | STAT1 | signal transducer and activator of transcription 1, 91 kDa | 1.061838965 | 0.006707 |
| 205050_s_at | MAPK8IP2 | mitogen-activated protein kinase 8 interacting protein 2 | 1.052857019 | 0.000511 |
| 217834_s_at | SYNCRIP | synaptotagmin binding, cytoplasmic RNA interacting protein | 1.038522393 | 0.005806 |
| 204192_at | CD37 | CD37 antigen | 1.029167905 | 0.00175 |
| 203421_at | TP53I11 | tumor protein p53 inducible protein 11 | 1.024354916 | 0.00015 |
| 205131_x_at | CLEC11A | C-type lectin domain family 11, member A | 1.023564752 | 0.006935 |
| 65133_i_at | ZNHIT4 | zinc finger, HIT type 4 | 1.01215222 | 0.005505 |
| 205312_at | SPI1 | spleen focus forming virus (SFFV) proviral integration oncogene spi1 | 1.008672462 | 0.000102 |
| 209266_s_at | SLC39A8 | solute carrier family 39 (zinc transporter), member 8 | 1.007342042 | 0.008898 |
| 206782_s_at | DNAJC4 | DnaJ (Hsp40) homolog, subfamily C, member 4 | 1.00438868 | 0.005432 |
| 210776_x_at | TCF3 | transcription factor 3 (E2A immunoglobulin enhancer binding factors | 0.984301782 | 0.002414 |
| | | E12/E47) | | |
| 205641_s_at | TRADD | TNFRSF1A-associated via death domain | 0.981998647 | 0.00963 |
| 215616_s_at | JMJD2B | jumonji domain containing 2B | 0.974423755 | 0.005036 |
| 218524_at | E4F1 | E4F transcription factor 1 | 0.972090779 | 0.00304 |
| 209166_s_at | MAN2B1 | mannosidase, alpha, class 2B, member 1 | 0.965388011 | 0.00909 |
| 210783_x_at | CLEC11A | C-type lectin domain family 11, member A | 0.964623024 | 0.00825 |
| 212312_at | BCL2L1 | BCL2-like 1 /// BCL2-like 1 | 0.956568938 | 0.003998 |
| 201309_x_at | C5orf13 | chromosome 5 open reading frame 13 | 0.943074822 | 0.009672 |
| 211622_s_at | ARF3 | ADP-ribosylation factor 3 /// ADP-ribosylation factor 3 | 0.940173907 | 0.002443 |
| 221525_at | DKFZp761I2123 | hypothetical protein DKFZp761I2123 | 0.931597762 | 3.08E−06 |
| 219040_at | CORO7 | coronin 7 | 0.931315331 | 0.000566 |
| 210862_s_at | SARDH | sarcosine dehydrogenase | 0.925137006 | 0.000327 |
| 218900_at | CNNM4 | cyclin M4 | 0.916639249 | 0.001065 |
| 218161_s_at | CLN6 | ceroid-lipofuscinosis, neuronal 6, late infantile, variant | 0.902839478 | 0.008702 |
| 1729_at | TRADD | TNFRSF1A-associated via death domain | 0.901121935 | 0.007888 |
| 204876_at | ZNF646 | zinc finger protein 646 | 0.8981329 | 0.0083 |
| 205165_at | CELSR3 | cadherin, EGF LAG seven-pass G-type receptor 3 (flamingo homolog, | 0.896931251 | 0.003155 |
| | | Drosophila) | | |
| 201806_s_at | ATXN2L | ataxin 2-like | 0.882606769 | 0.002438 |
| 209906_at | C3AR1 | complement component 3a receptor 1 | 0.880463808 | 0.003461 |
| 218151_x_at | GPR172A | G protein-coupled receptor 172A | 0.876852613 | 0.002933 |
| 209973_at | NFKBIL1 | nuclear factor of kappa light polypeptide gene enhancer in B-cells | 0.869189181 | 0.003246 |
| | | inhibitor-like 1 | | |
| 201755_at | MCM5 | MCM5 minichromosome maintenance deficient 5, cell division cycle 46 | 0.86573888 | 0.005865 |
| | | (S. cerevisiae) | | |
| 218060_s_at | FLJ13154 | hypothetical protein FLJ13154 | 0.861692665 | 0.008244 |
| 34260_at | KIAA0683 | KIAA0683 gene product | 0.861468194 | 0.002057 |
| 219227_at | FLJ14166 | hypothetical protein FLJ14166 | 0.844958683 | 0.00054 |
| 206179_s_at | TPPP | brain-specific protein p25 alpha | 0.840038822 | 0.006931 |
| 220408_x_at | FAM48A | family with sequence similarity 48, member A | 0.834810375 | 0.003026 |
| 205459_s_at | NPAS2 | neuronal PAS domain protein 2 | 0.834483527 | 0.00215 |
| 202828_s_at | MMP14 | matrix metallopeptidase 14 (membrane-inserted) | 0.832258469 | 0.008966 |
| 215660_s_at | MAST2 | microtubule associated serine/threonine kinase 2 | 0.825371167 | 0.006286 |
| 217399_s_at | FOXO3A | forkhead box O3A | 0.818163753 | 0.006906 |
| 203713_s_at | LLGL2 | lethal giant larvae homolog 2 (Drosophila) | 0.806116539 | 0.00397 |
| 218778_x_at | EPS8L1 | EPS8-like 1 | 0.804607035 | 0.004115 |
| 217635_s_at | POLG | polymerase (DNA directed), gamma | 0.79874289 | 0.008862 |
| 207484_s_at | EHMT2 | euchromatic histone-lysine N-methyltransferase 2 | 0.794528405 | 0.001093 |
| 217318_x_at | KIR3DL2 /// | killer cell immunoglobulin-like receptor, three domains, long cytoplasmic | 0.785885277 | 0.007021 |
| | KIR2DL5A /// | tail, 2 /// killer cell immunoglobulin-like receptor, two domains, long | | |
| | KIR3DL3 /// | cytoplasmic tail, 5A /// killer cell immunoglobulin-like receptor, three | | |
| | KIR3DP1 /// | domains, long cytoplasmic tail, 3 /// killer cell immunoglobulin-like | | |
| | KIR2DL5B | receptor, three domains, pseudogene 1 /// killer cell immunoglobulin-like | | |
| | | receptor, two domains, long cytoplasmic tail, 5B | | |
| 216269_s_at | ELN | elastin (supravalvular aortic stenosis, Williams-Beuren syndrome) | 0.782926943 | 0.004981 |
| 203110_at | PTK2B | PTK2B protein tyrosine kinase 2 beta | 0.782294713 | 0.00925 |
| 221956_at | LRCH4 | leucine-rich repeats and calponin homology (CH) domain containing 4 | 0.776309624 | 0.000405 |
| 209346_s_at | PI4KII | phosphatidylinositol 4-kinase type II | 0.776079 | 0.00567 |
| 213772_s_at | GGA2 | golgi associated, gamma adaptin ear containing, ARF binding protein 2 | 0.775961201 | 0.000385 |
| 205056_s_at | GPR162 | G protein-coupled receptor 162 | 0.772934808 | 0.004891 |
| 221827_at | C20orf18 | chromosome 20 open reading frame 18 | 0.770810095 | 0.008335 |
| 211631_x_at | B4GALT1 | UDP-Gal:betaGlcNAc beta 1,4-galactosyltransferase, polypeptide 1 /// | 0.769453748 | 0.006482 |
| | | UDP-Gal:betaGlcNAc beta 1,4-galactosyltransferase, polypeptide 1 | | |
| 204561_x_at | APOC2 | apolipoprotein C-II | 0.768790428 | 0.006691 |
| 209606_at | PSCDBP | pleckstrin homology, Sec7 and coiled-coil domains, binding protein /// | 0.767819454 | 0.000536 |
| | | pleckstrin homology, Sec7 and coiled-coil domains, binding protein | | |
| 204654_s_at | TFAP2A | transcription factor AP-2 alpha (activating enhancer binding protein 2 | 0.765548068 | 0.005104 |
| | | alpha) | | |
| 215501_s_at | DUSP10 | dual specificity phosphatase 10 | 0.764372765 | 0.001137 |
| 201638_s_at | CPSF1 | cleavage and polyadenylation specific factor 1, 160 kDa | 0.760097664 | 0.0073 |
| 208110_x_at | MED25 | mediator of RNA polymerase II transcription, subunit 25 homolog (yeast) | 0.754612516 | 0.004973 |
| | | /// mediator of RNA polymerase II transcription, subunit 25 homolog | | |
| | | (yeast) | | |
| 203904_x_at | CD82 | CD82 antigen | 0.75448255 | 0.002761 |
| 218033_s_at | SNN | stannin | 0.75046509 | 0.003139 |
| 221819_at | RAB35 | RAB35, member RAS oncogene family | 0.750420795 | 0.002767 |
| 204740_at | CNKSR1 | connector enhancer of kinase suppressor of Ras 1 | 0.733951109 | 0.00534 |
| 207684_at | TBX6 | T-box 6 | 0.733730783 | 0.001236 |
| 217239_x_at | — | Similar to immunoglobulin M chain /// (fetal) Ig rearranged H-chain VDJ- | 0.729465376 | 0.004266 |
| | | region mRNA, clone 60P2 /// Immunoglobulin heavy chain variable | | |
| | | region (VH III family) from IgM rheumatoid factor | | |
| 222206_s_at | NCLN | nicalin homolog (zebrafish) | 0.726650572 | 0.003808 |
| 221769_at | SPSB3 | splA/ryanodine receptor domain and SOCS box containing 3 | 0.714802911 | 0.008419 |
| 203665_at | HMOX1 | heme oxygenase (decycling) 1 | 0.713086293 | 0.003608 |
| 216397_s_at | BOP1 | block of proliferation 1 | 0.710439053 | 0.00661 |
| 216486_x_at | ZNF79 | zinc finger protein 79 (pT7) | 0.70278943 | 0.00744 |
| 210618_at | RAP1GA1 | RAP1, GTPase activating protein 1 | 0.701787237 | 0.008246 |
| 220279_at | TRIM17 | tripartite motif-containing 17 | 0.701696588 | 0.005732 |
| 212860_at | ZDHHC18 | zinc finger, DHHC-type containing 18 | 0.692179196 | 0.001172 |
| 206397_x_at | GDF1 /// LASS1 | growth differentiation factor 1 /// LAG1 longevity assurance homolog 1 | 0.681040048 | 0.00893 |
| | | (S. cerevisiae) | | |
| 216253_s_at | PARVB | parvin, beta | 0.68008837 | 0.004465 |
| 203388_at | ARRB2 | arrestin, beta 2 | 0.676275378 | 0.008795 |
| 212909_at | LYPD1 | LY6/PLAUR domain containing 1 | 0.66440939 | 0.007554 |
| 220565_at | CCR10 | chemokine (C—C motif) receptor 10 | 0.654341383 | 0.00317 |
| 202835_at | TXNL4A | thioredoxin-like 4A | 0.639106937 | 0.008589 |
| 209664_x_at | NFATC1 | nuclear factor of activated T-cells, cytoplasmic, calcineurin-dependent 1 | 0.634834568 | 0.005169 |
| 217348_x_at | ARHGEF15 | Rho guanine nucleotide exchange factor (GEF) 15 | 0.629597609 | 0.009076 |
| 221964_at | TULP3 | tubby like protein 3 | 0.586438249 | 0.005707 |
| 212541_at | FLAD1 | Fad1, flavin adenine dinucleotide synthetase, homolog (yeast) | 0.582246433 | 0.008041 |
| 216928_at | TAL1 | T-cell acute lymphocytic leukemia 1 | 0.552267983 | 0.009048 |
| 207129_at | CA5B | carbonic anhydrase VB, mitochondrial | 0.548399587 | 0.005982 |
| 222226_at | SAA3P | serum amyloid A3 pseudogene | 0.540537928 | 0.004708 |
| 212210_at | DKFZP586J0619 | DKFZP586J0619 protein | 0.531909885 | 0.007105 |
| 221302_at | KLF15 | Kruppel-like factor 15 | 0.529771199 | 0.006305 |
| 214154_s_at | PKP2 | plakophilin 2 | 0.515217352 | 0.009465 |
| 219794_at | FLJ10979 | hypothetical protein FLJ10979 | 0.497258115 | 0.001629 |
| 221605_s_at | PIPOX | pipecolic acid oxidase | −0.50431013 | 0.008656 |
| 209640_at | PML | promyelocytic leukemia | −0.55944007 | 0.004261 |
| 210197_at | ITPK1 | inositol 1,3,4-triphosphate 5/6 kinase | −0.57382402 | 0.002728 |
| 204627_s_at | ITGB3 | integrin, beta 3 (platelet glycoprotein IIIa, antigen CD61) | −0.63106292 | 0.004923 |
| 207776_s_at | CACNB2 | calcium channel, voltage-dependent, beta 2 subunit | −0.65065517 | 0.00724 |
| 205743_at | STAC | SH3 and cysteine rich domain | −0.65441404 | 0.005835 |
| 218515_at | C21orf66 | Chromosome 21 open reading frame 66 | −0.65673727 | 0.004143 |
| 213556_at | LOC390940 | similar to R28379_1 | −0.67610942 | 0.00278 |
| 204101_at | MTM1 | myotubularin 1 | −0.68206735 | 0.008859 |
| 202783_at | NNT | nicotinamide nucleotide transhydrogenase | −0.68667172 | 0.006744 |
| 206648_at | ZNF571 | zinc finger protein 571 | −0.7028254 | 0.005997 |
| 203339_at | SLC25A12 | solute carrier family 25 (mitochondrial carrier, Aralar), member 12 | −0.7041933 | 0.009364 |
| 205610_at | MYOM1 | myomesin 1 (skelemin) 185 kDa | −0.71121509 | 0.006047 |
| 221347_at | CHRM5 | cholinergic receptor, muscarinic 5 | −0.71869285 | 0.002223 |
| 221883_at | PKNOX1 | PBX/knotted 1 homeobox 1 | −0.72499071 | 0.008725 |
| 210751_s_at | RGN | regucalcin (senescence marker protein-30) | −0.73027121 | 0.00376 |
| 214945_at | NY-REN-7 /// | NY-REN-7 antigen /// similar to KIAA0752 protein | −0.730904 | 0.005715 |
| | LOC389347 | | | |
| 203661_s_at | TMOD1 | tropomodulin 1 | −0.74604145 | 0.003351 |
| 221044_s_at | TRIM34 /// | tripartite motif-containing 34 /// tripartite motif-containing 6 and tripartite | −0.74753844 | 0.007554 |
| | TRIM6-TRIM34 | motif-containing 34 | | |
| 203067_at | PDHX | pyruvate dehydrogenase complex, component X | −0.76522922 | 0.007264 |
| 213149_at | DLAT | dihydrolipoamide S-acetyltransferase (E2 component of pyruvate | −0.77065763 | 0.008295 |
| | | dehydrogenase complex) | | |
| 219217_at | FLJ23441 | hypothetical protein FLJ23441 | −0.82350455 | 0.004043 |
| 222184_at | — | Clone 25015 mRNA sequence | −0.82451004 | 0.005988 |
| 219464_at | CA14 | carbonic anhydrase XIV | −0.85151131 | 0.001788 |
| 203678_at | KIAA1018 | KIAA1018 protein | −0.85238538 | 0.008184 |
| 203493_s_at | PIG8 | translokin | −0.86026192 | 0.009846 |
| 218284_at | SMAD3 | SMAD, mothers against DPP homolog 3 (Drosophila) | −0.86334284 | 0.007209 |
| 203340_s_at | SLC25A12 | solute carrier family 25 (mitochondrial carrier, Aralar), member 12 | −0.86434437 | 0.003291 |
| 212549_at | STAT5B | signal transducer and activator of transcription 5B | −0.89053653 | 0.000704 |
| 202520_s_at | MLH1 | mutL homolog 1, colon cancer, nonpolyposis type 2 (E. coli) | −0.89205598 | 0.002419 |
| 219292_at | THAP1 | THAP domain containing, apoptosis associated protein 1 | −0.9081982 | 0.003527 |
| 203295_s_at | ATP1A2 | ATPase, Na+/K+ transporting, alpha 2 (+) polypeptide | −0.91450659 | 0.005197 |
| 205088_at | CXorf6 | chromosome X open reading frame 6 | −0.93252411 | 0.006048 |
| 213455_at | LOC283677 | hypothetical LOC283677 | −0.93533955 | 0.004083 |
| 204273_at | EDNRB | endothelin receptor type B | −0.95041426 | 0.001573 |
| 213661_at | DKFZP586H2123 | regeneration associated muscle protease | −0.97931329 | 0.000103 |
| 214660_at | PELO | Pelota homolog (Drosophila) | −0.99965707 | 0.001013 |
| 209314_s_at | HBS1L | HBS1-like (S. cerevisiae) | −1.01951087 | 0.003451 |
| 218043_s_at | AZI2 | 5-azacytidine induced 2 | −1.02481557 | 0.009637 |
| 217721_at | 7-Sep | Septin 7 | −1.02559647 | 1.04E−05 |
| 222150_s_at | LOC54103 | hypothetical protein LOC54103 | −1.03784254 | 0.000709 |
| 212297_at | ATP13A3 | ATPase type 13A3 | −1.04040016 | 0.003333 |
| 205348_s_at | DNCI1 | dynein, cytoplasmic, intermediate polypeptide 1 | −1.04536482 | 0.004605 |
| 216944_s_at | ITPR1 | inositol 1,4,5-triphosphate receptor, type 1 | −1.05338499 | 0.002504 |
| 212757_s_at | CAMK2G | calcium/calmodulin-dependent protein kinase (CaM kinase) II gamma | −1.05360528 | 0.003609 |
| 219764_at | FZD10 | frizzled homolog 10 (Drosophila) | −1.06313139 | 0.008364 |
| 205355_at | ACADSB | acyl-Coenzyme A dehydrogenase, short/branched chain | −1.07225397 | 0.00024 |
| 211423_s_at | SC5DL | sterol-C5-desaturase (ERG3 delta-5-desaturase homolog, fungal)-like | −1.09101617 | 0.00355 |
| 218842_at | FLJ21908 | hypothetical protein FLJ21908 | −1.09131478 | 0.009608 |
| 201517_at | NCBP2 | nuclear cap binding protein subunit 2, 20 kDa | −1.09569572 | 0.003334 |
| 202629_at | APPBP2 | amyloid beta precursor protein (cytoplasmic tail) binding protein 2 | −1.09597872 | 0.006989 |
| 203368_at | CRELD1 | cysteine-rich with EGF-like domains 1 | −1.10296371 | 0.006024 |
| 218926_at | MYNN | myoneurin | −1.10682045 | 0.005249 |
| 203102_s_at | MGAT2 | mannosyl (alpha-1,6-)-glycoprotein beta-1,2-N- | −1.12479778 | 0.009853 |
| | | acetylglucosaminyltransferase | | |
| 204640_s_at | SPOP | speckle-type POZ protein | −1.14057396 | 0.008894 |
| 209085_x_at | RFC1 | replication factor C (activator 1) 1, 145 kDa | −1.14678312 | 0.009435 |
| 216887_s_at | LDB3 | LIM domain binding 3 | −1.15951792 | 6.69E−05 |
| 218446_s_at | FAM18B | family with sequence similarity 18, member B | −1.19486247 | 0.008233 |
| 203607_at | INPP5F | inositol polyphosphate-5-phosphatase F | −1.21721914 | 0.00778 |
| 208962_s_at | FADS1 | fatty acid desaturase 1 | −1.23095952 | 0.009261 |
| 203301_s_at | DMTF1 | cyclin D binding myb-like transcription factor 1 | −1.23757435 | 0.002026 |
| 209616_s_at | CES1 | carboxylesterase 1 (monocyte/macrophage serine esterase 1) | −1.23772255 | 0.006259 |
| 205091_x_at | RECQL | RecQ protein-like (DNA helicase Q1-like) | −1.26382967 | 0.002155 |
| 212408_at | TOR1AIP1 | torsin A interacting protein 1 | −1.26884999 | 0.000794 |
| 209115_at | UBE1C | ubiquitin-activating enzyme E1C (UBA3 homolog, yeast) | −1.28406789 | 0.006194 |
| 205609_at | ANGPT1 | angiopoietin 1 | −1.29995843 | 0.000936 |
| 221556_at | CDC14B | CDC14 cell division cycle 14 homolog B (S. cerevisiae) | −1.33129421 | 0.000541 |
| 212470_at | SPAG9 | sperm associated antigen 9 | −1.33265021 | 0.001111 |
| 218356_at | FTSJ2 | FtsJ homolog 2 (E. coli) | −1.33926239 | 0.009562 |
| 203359_s_at | MYCBP | c-myc binding protein | −1.35166348 | 0.001626 |
| 217976_s_at | DNCLI1 | dynein, cytoplasmic, light intermediate polypeptide 1 | −1.35231538 | 0.006603 |
| 202364_at | MXI1 | MAX interactor 1 /// MAX interactor 1 | −1.37647331 | 0.007104 |
| 218285_s_at | DHRS6 | dehydrogenase/reductase (SDR family) member 6 | −1.39658249 | 0.009223 |
| 203066_at | GALNAC4S-6ST | B cell RAG associated protein | −1.39669919 | 0.002459 |
| 203638_s_at | FGFR2 | fibroblast growth factor receptor 2 (bacteria-expressed kinase, | −1.39817144 | 0.003227 |
| | | keratinocyte growth factor receptor, craniofacial dysostosis 1, Crouzon | | |
| | | syndrome, Pfeiffer syndrome, Jackson-Weiss syndrome) | | |
| 202566_s_at | SVIL | supervillin | −1.41043106 | 0.003067 |
| 202666_s_at | ACTL6A | actin-like 6A | −1.41181602 | 0.00432 |
| 217645_at | C14orf112 | Chromosome 14 open reading frame 112 | −1.42306469 | 0.001156 |
| 214464_at | CDC42BPA | CDC42 binding protein kinase alpha (DMPK-like) | −1.42358933 | 0.007193 |
| 218583_s_at | DCUN1D1 | DCN1, defective in cullin neddylation 1, domain containing 1 (S. cerevisiae) | −1.42862207 | 8.49E−05 |
| 218696_at | EIF2AK3 | eukaryotic translation initiation factor 2-alpha kinase 3 | −1.43324674 | 0.002507 |
| 218458_at | GCL | germ cell-less homolog 1 (Drosophila) | −1.43562848 | 0.000817 |
| 204847_at | ZBTB11 | zinc finger and BTB domain containing 11 | −1.45652565 | 0.003812 |
| 212508_at | MOAP1 | modulator of apoptosis 1 | −1.46228963 | 0.009805 |
| 209512_at | HSDL2 | hydroxysteroid dehydrogenase like 2 | −1.46513595 | 0.005694 |
| 221559_s_at | MIS12 | MIS12 homolog (yeast) | −1.48224324 | 0.005882 |
| 208857_s_at | PCMT1 | protein-L-isoaspartate (D-aspartate) O-methyltransferase | −1.51253251 | 0.00136 |
| 204496_at | STRN3 | striatin, calmodulin binding protein 3 | −1.51677917 | 6.66E−07 |
| 209248_at | GHITM | growth hormone inducible transmembrane protein | −1.53299358 | 0.004751 |
| 205501_at | — | CDNA FLJ25677 fis, clone TST04054 | −1.54125794 | 0.004143 |
| 217894_at | KCTD3 | potassium channel tetramerisation domain containing 3 | −1.54669027 | 0.00242 |
| 218432_at | FBXO3 | F-box protein 3 | −1.55082805 | 0.006021 |
| 212476_at | CENTB2 | centaurin, beta 2 | −1.57248456 | 0.000834 |
| 213111_at | PIP5K3 | phosphatidylinositol-3-phosphate/phosphatidylinositol 5-kinase, type III | −1.59084559 | 0.00612 |
| 220595_at | PDZRN4 | PDZ domain containing RING finger 4 | −1.59949653 | 0.002427 |
| 205202_at | PCMT1 | protein-L-isoaspartate (D-aspartate) O-methyltransferase | −1.60114799 | 0.00871 |
| 213510_x_at | LOC220594 | TL132 protein | −1.60917878 | 0.004543 |
| 214110_s_at | — | Similar to lymphocyte-specific protein 1 | −1.61029591 | 0.005937 |
| 208990_s_at | HNRPH3 | heterogeneous nuclear ribonucleoprotein H3 (2H9) | −1.616026 | 0.002598 |
| 221543_s_at | SPFH2 | SPFH domain family, member 2 | −1.63311048 | 0.008106 |
| 205407_at | RECK | reversion-inducing-cysteine-rich protein with kazal motifs | −1.63514289 | 0.009982 |
| 213331_s_at | NEK1 | NIMA (never in mitosis gene a)-related kinase 1 | −1.64214114 | 0.004306 |
| 212806_at | KIAA0367 | KIAA0367 | −1.67732615 | 0.008455 |
| 208927_at | SPOP | speckle-type POZ protein | −1.69297515 | 0.000958 |
| 204793_at | GPRASP1 | G protein-coupled receptor associated sorting protein 1 | −1.72462939 | 0.007174 |
| 209005_at | FBXL5 | F-box and leucine-rich repeat protein 5 | −1.7915898 | 0.005041 |
| 218499_at | MASK | Mst3 and SOK1-related kinase | −1.81965417 | 0.00809 |
| 213714_at | CACNB2 | calcium channel, voltage-dependent, beta 2 subunit | −1.86889018 | 0.001614 |
| 218683_at | PTBP2 | polypyrimidine tract binding protein 2 | −1.88451684 | 0.002306 |
| 217776_at | RDH11 | retinol dehydrogenase 11 (all-trans and 9-cis) | −1.88682983 | 0.005793 |
| 213371_at | LDB3 | LIM domain binding 3 | −1.89439777 | 0.000105 |
| 219685_at | TMEM35 | transmembrane protein 35 | −2.01663255 | 0.004303 |
| 212397_at | RDX | radixin | −2.01924579 | 0.006075 |
| 205083_at | AOX1 | aldehyde oxidase 1 | −2.1175354 | 0.001062 |
| 221553_at | DKFZp564K142 | implantation-associated protein | −2.16829747 | 0.000416 |
| 204412_s_at | NEFH | neurofilament, heavy polypeptide 200 kDa | −2.18137802 | 0.006076 |
| 201617_x_at | CALD1 | caldesmon 1 | −2.40787957 | 0.00202 |
| 209763_at | CHRDL1 | chordin-like 1 | −2.61605999 | 0.001164 |
| 217775_s_at | RDH11 | retinol dehydrogenase 11 (all-trans and 9-cis) | −2.71917266 | 0.002765 |
| 212530_at | NEK7 | NIMA (never in mitosis gene a)-related kinase 7 | −2.86398048 | 3.07E−05 |
| 33767_at | NEFH | neurofilament, heavy polypeptide 200 kDa | −3.4120129 | 0.005992 |
| 203946_s_at | ARG2 | arginase, type II | −3.7410222 | 0.008657 |

TABLE 36
dataset 1 (tumor)

| | | | log base | |
|---|---|---|---|---|
| | | | 2 fold | |
| uniqueID | Gene Symbol | Gene Title | change | p value |
| 214455_at | HIST1H2BC | histone 1, H2bc | 4.358572 | 0.000901 |
| 214469_at | HIST1H2AE | histone 1, H2ae | 4.062953 | 0.00297 |
| 205347_s_at | TMSL8 | thymosin-like 8 | 3.770383 | 0.001191 |
| 205009_at | TFF1 | trefoil factor 1 (breast cancer, estrogen-inducible | 3.595782 | 0.004612 |
| | | sequence expressed in) | | |
| 215779_s_at | HIST1H2BG | histone 1, H2bg | 3.42953 | 4.59E−05 |
| 218280_x_at | HIST2H2AA | histone 2, H2aa | 2.907689 | 8.37E−05 |
| AFFX-r2-Ec- | bioB | biotin synthesis, sulfur insertion? | 2.742559 | 0.002921 |
| bioB-M_at | | | | |
| 214472_at | HIST1H3D | histone 1, H3d | 2.69731 | 0.009665 |
| 205501_at | — | CDNA FLJ25677 fis, clone TST04054 | 2.609067 | 0.001198 |
| 203164_at | SLC33A1 | solute carrier family 33 (acetyl-CoA transporter), member 1 | 2.565196 | 0.001372 |
| 208180_s_at | HIST1H4H | histone 1, H4h | 2.558291 | 0.000912 |
| 207076_s_at | ASS | argininosuccinate synthetase | 2.342411 | 0.000472 |
| AFFX-r2-Ec- | bioB | Biotin synthase /// biotin synthesis, sulfur insertion? | 2.167319 | 0.006457 |
| bioB-3_at | | | | |
| AFFX-BioB- | bioB | Biotin synthase /// biotin synthesis, sulfur insertion? | 2.150147 | 0.004866 |
| M_at | | | | |
| 201962_s_at | RNF41 | ring finger protein 41 | 2.095234 | 0.001988 |
| AFFX-r2-Ec- | bioC | biotin biosynthesis; reaction prior to pimeloyl CoA | 2.030088 | 0.004128 |
| bioC-5_at | | | | |
| 206110_at | HIST1H3H | histone 1, H3h | 1.973389 | 0.001869 |
| 214683_s_at | CLK1 | CDC-like kinase 1 | 1.967806 | 0.002233 |
| 222067_x_at | HIST1H2BD | histone 1, H2bd | 1.967367 | 4.18E−05 |
| 208546_x_at | HIST1H2BH | histone 1, H2bh | 1.956574 | 9.08E−06 |
| 214290_s_at | HIST2H2AA | histone 2, H2aa | 1.926708 | 0.000733 |
| 200888_s_at | RPL23 | ribosomal protein L23 | 1.901666 | 0.002246 |
| AFFX-BioC- | bioC | biotin biosynthesis; reaction prior to pimeloyl CoA | 1.854454 | 0.00595 |
| 3_at | | | | |
| 201636_at | FXR1 | fragile X mental retardation, autosomal homolog 1 | 1.850738 | 1.92E−05 |
| 201031_s_at | HNRPH1 | heterogeneous nuclear ribonucleoprotein H1 (H) | 1.844054 | 0.001705 |
| AFFX-r2-Ec- | bioC | biotin biosynthesis; reaction prior to pimeloyl CoA | 1.804444 | 0.004125 |
| bioC-3_at | | | | |
| 218085_at | CHMP5 | chromatin modifying protein 5 | 1.775694 | 0.002059 |
| 202666_s_at | ACTL6A | actin-like 6A | 1.772138 | 0.001759 |
| 213704_at | RABGGTB | Rab geranylgeranyltransferase, beta subunit | 1.746765 | 0.006789 |
| 202903_at | LSM5 | LSM5 homolog, U6 small nuclear RNA associated (S. cerevisiae) | 1.691493 | 0.00515 |
| 207543_s_at | P4HA1 | procollagen-proline, 2-oxoglutarate 4-dioxygenase | 1.659193 | 0.008062 |
| | | (proline 4-hydroxylase), alpha polypeptide I | | |
| 202425_x_at | PPP3CA | protein phosphatase 3 (formerly 2B), catalytic subunit, | 1.647563 | 0.008555 |
| | | alpha isoform (calcineurin A alpha) | | |
| 208490_x_at | HIST1H2BF | histone 1, H2bf | 1.637573 | 0.000595 |
| 216177_at | LOC283412 /// | similar to 60S ribosomal protein L29 (Cell surface heparin | 1.635539 | 0.001375 |
| | LOC284064 | binding protein HIP) /// similar to 60S ribosomal protein | | |
| | | L29 (Cell surface heparin binding protein HIP) | | |
| 215088_s_at | SDHC | succinate dehydrogenase complex, subunit C, integral | 1.624131 | 0.00957 |
| | | membrane protein, 15 kDa | | |
| 218268_at | TBC1D15 | TBC1 domain family, member 15 | 1.597015 | 0.008018 |
| 209248_at | GHITM | growth hormone inducible transmembrane protein | 1.572931 | 0.003096 |
| 219375_at | CEPT1 | choline/ethanolamine phosphotransferase 1 | 1.551122 | 0.001931 |
| 217850_at | GNL3 | guanine nucleotide binding protein-like 3 (nucleolar) | 1.533389 | 0.00342 |
| 217775_s_at | RDH11 | retinol dehydrogenase 11 (all-trans and 9-cis) | 1.531528 | 0.00246 |
| 208523_x_at | HIST1H2BI | histone 1, H2bi | 1.499348 | 1.43E−05 |
| 201407_s_at | PPP1CB | protein phosphatase 1, catalytic subunit, beta isoform | 1.488251 | 0.004231 |
| 214522_x_at | HIST1H2AD /// | histone 1, H2ad /// histone 1, H3d | 1.485638 | 0.003751 |
| | HIST1H3D | | | |
| 218643_s_at | CRIPT | postsynaptic protein CRIPT | 1.485566 | 0.006706 |
| 218605_at | TFB2M | transcription factor B2, mitochondrial | 1.482999 | 0.002284 |
| 206100_at | CPM | carboxypeptidase M | 1.480325 | 0.008133 |
| 215071_s_at | HIST1H2AC | histone 1, H2ac | 1.461777 | 0.000361 |
| 208527_x_at | HIST1H2BE | histone 1, H2be | 1.449344 | 0.003147 |
| 208828_at | POLE3 | polymerase (DNA directed), epsilon 3 (p17 subunit) | 1.408762 | 0.005599 |
| 209797_at | TMEM4 | transmembrane protein 4 | 1.399449 | 0.005354 |
| 219048_at | PIGN | phosphatidylinositol glycan, class N | 1.303567 | 0.005323 |
| 209911_x_at | HIST1H2BD | histone 1, H2bd | 1.281097 | 0.000582 |
| 219889_at | FRAT1 | frequently rearranged in advanced T-cell lymphomas | 1.228008 | 0.005585 |
| 217368_at | — | — | 1.204159 | 0.005441 |
| 214542_x_at | HIST1H2AI | histone 1, H2ai | 1.201637 | 0.001861 |
| 218583_s_at | DCUN1D1 | DCN1, defective in cullin neddylation 1, domain | 1.195575 | 0.003238 |
| | | containing 1 (S. cerevisiae) | | |
| 208646_at | RPS14 | ribosomal protein S14 | 1.161299 | 0.003706 |
| 205355_at | ACADSB | acyl-Coenzyme A dehydrogenase, short/branched chain | 1.155481 | 0.004407 |
| 201542_at | SAR1A | SAR1 gene homolog A (S. cerevisiae) | 1.152993 | 0.008667 |
| 222279_at | FLJ35429 | hypothetical protein FLJ35429 | 1.146027 | 0.001236 |
| AFFX-r2-P1- | — | — | 1.14421 | 0.009331 |
| cre-3_at | | | | |
| 214481_at | HIST1H2AM | Histone 1, H2am | 1.124449 | 0.000726 |
| 54632_at | THADA | thyroid adenoma associated | 1.11474 | 0.006338 |
| 202244_at | PSMB4 | proteasome (prosome, macropain) subunit, beta type, 4 | 1.109895 | 0.004437 |
| 209755_at | NMNAT2 | nicotinamide nucleotide adenylyltransferase 2 | 1.090024 | 0.009247 |
| 201456_s_at | BUB3 | BUB3 budding uninhibited by benzimidazoles 3 homolog | 1.077827 | 0.008934 |
| | | (yeast) | | |
| 218926_at | MYNN | myoneurin | 1.074881 | 0.009357 |
| 202361_at | SEC24C | SEC24 related gene family, member C (S. cerevisiae) | 1.00409 | 0.009599 |
| 218882_s_at | WDR3 | WD repeat domain 3 | 0.998624 | 0.004399 |
| 211423_s_at | SC5DL | sterol-C5-desaturase (ERG3 delta-5-desaturase homolog, | 0.997797 | 0.007958 |
| | | fungal)-like | | |
| 204496_at | STRN3 | striatin, calmodulin binding protein 3 | 0.989084 | 0.003394 |
| 220445_s_at | CSAG2 | CSAG family, member 2 | 0.956467 | 0.003469 |
| 202029_x_at | RPL38 | ribosomal protein L38 | 0.932832 | 0.003047 |
| 218712_at | C1orf109 | chromosome 1 open reading frame 109 | 0.924297 | 0.002317 |
| 219940_s_at | FLJ11305 | hypothetical protein FLJ11305 | 0.906085 | 0.003188 |
| 218615_s_at | TMEM39A | transmembrane protein 39A | 0.905335 | 0.008103 |
| 202231_at | hfl-B5 | dendritic cell protein | 0.895731 | 0.007247 |
| 213253_at | SMC2L1 | SMC2 structural maintenance of chromosomes 2-like 1 | 0.881586 | 0.007795 |
| | | (yeast) | | |
| 217347_at | — | — | 0.879581 | 0.003568 |
| 217598_at | CINP | Cyclin-dependent kinase 2-interacting protein | 0.860204 | 0.00932 |
| 200043_at | ERH | enhancer of rudimentary homolog (Drosophila) /// | 0.858756 | 0.002082 |
| | | enhancer of rudimentary homolog (Drosophila) | | |
| 216382_s_at | TNRC21 | trinucleotide repeat containing 21 | 0.847784 | 0.007086 |
| 200025_s_at | RPL27 | ribosomal protein L27 /// ribosomal protein L27 | 0.817494 | 0.008265 |
| 204120_s_at | ADK | adenosine kinase | 0.786247 | 0.002474 |
| 218598_at | RINT-1 | Rad50-interacting protein 1 | 0.77458 | 0.008568 |
| 201377_at | UBAP2L | ubiquitin associated protein 2-like | 0.76542 | 0.004964 |
| 217019_at | RPS4X | ribosomal protein S4, X-linked | 0.746504 | 0.00445 |
| 214583_at | RSC1A1 | regulatory solute carrier protein, family 1, member 1 | 0.731596 | 0.00277 |
| 220167_s_at | TP53TG3 | TP53TG3 protein | 0.723961 | 0.006065 |
| 216317_x_at | RHCE | Rhesus blood group, CcEe antigens | 0.72354 | 0.005877 |
| 215819_s_at | RHCE /// RHD | Rhesus blood group, CcEe antigens /// Rhesus blood | 0.704674 | 0.003491 |
| | | group, D antigen | | |
| 221465_at | OR6A2 | olfactory receptor, family 6, subfamily A, member 2 | 0.698869 | 0.005448 |
| 218104_at | TEX10 | testis expressed sequence 10 | 0.697213 | 0.007688 |
| 219464_at | CA14 | carbonic anhydrase XIV | 0.675065 | 0.003769 |
| 204191_at | IFNAR1 | interferon (alpha, beta and omega) receptor 1 | 0.646818 | 0.008478 |
| 213903_s_at | RQCD1 | RCD1 required for cell differentiation1 homolog (S. pombe) | 0.634636 | 0.007719 |
| 208241_at | NRG1 | neuregulin 1 | 0.612895 | 0.009468 |
| 219711_at | ZNF586 | zinc finger protein 586 | 0.588872 | 0.003791 |
| 210315_at | SYN2 | synapsin II | 0.567586 | 0.008766 |
| 207418_s_at | DDO | D-aspartate oxidase | 0.546003 | 0.009539 |
| 210197_at | ITPK1 | inositol 1,3,4-triphosphate 5/6 kinase | 0.522487 | 0.007189 |
| 216643_at | MGC29898 | Hypothetical protein MGC29898 | 0.488018 | 0.006114 |
| 214599_at | IVL | involucrin | 0.479332 | 0.006939 |
| 213888_s_at | TRAF3IP3 | TRAF3 interacting protein 3 | −0.51908 | 0.002587 |
| 211217_s_at | KCNQ1 | potassium voltage-gated channel, KQT-like subfamily, | −0.54572 | 0.002446 |
| | | member 1 | | |
| 207238_s_at | PTPRC | protein tyrosine phosphatase, receptor type, C | −0.5739 | 0.009871 |
| 219677_at | SPSB1 | splA/ryanodine receptor domain and SOCS box | −0.61119 | 0.009945 |
| | | containing 1 | | |
| 213211_s_at | TAF6L | TAF6-like RNA polymerase II, p300/CBP-associated | −0.71299 | 0.008806 |
| | | factor (PCAF)-associated factor, 65 kDa | | |
| 215411_s_at | TRAF3IP2 | TRAF3 interacting protein 2 | −0.72938 | 0.003376 |
| 207334_s_at | TGFBR2 | transforming growth factor, beta receptor II (70/80 kDa) | −0.74428 | 0.005555 |
| 56197_at | PLSCR3 /// | phospholipid scramblase 3 /// hypothetical protein | −0.77096 | 0.000375 |
| | MGC40107 | MGC40107 | | |
| 221638_s_at | STX16 | syntaxin 16 | −0.77643 | 0.008744 |
| 211082_x_at | MARK2 | MAP/microtubule affinity-regulating kinase 2 /// | −0.77848 | 0.000404 |
| | | MAP/microtubule affinity-regulating kinase 2 | | |
| 217798_at | CNOT2 | CCR4-NOT transcription complex, subunit 2 | −0.78477 | 0.009738 |
| 213815_x_at | C19orf29 | chromosome 19 open reading frame 29 | −0.80837 | 0.003387 |
| 213209_at | TAF6L | TAF6-like RNA polymerase II, p300/CBP-associated | −0.80961 | 0.003306 |
| | | factor (PCAF)-associated factor, 65 kDa | | |
| 216237_s_at | MCM5 | MCM5 minichromosome maintenance deficient 5, cell | −0.8119 | 0.002492 |
| | | division cycle 46 (S. cerevisiae) | | |
| 215994_x_at | KIAA0676 | KIAA0676 protein | −0.82092 | 0.009066 |
| 219211_at | USP18 | ubiquitin specific peptidase 18 | −0.82139 | 0.002558 |
| 202180_s_at | MVP | major vault protein | −0.8218 | 0.003661 |
| 202594_at | LEPROTL1 | leptin receptor overlapping transcript-like 1 | −0.82262 | 0.005424 |
| 220319_s_at | MYLIP | myosin regulatory light chain interacting protein | −0.82297 | 0.006616 |
| 203167_at | TIMP2 | TIMP metallopeptidase inhibitor 2 | −0.8333 | 0.00033 |
| 212054_x_at | KIAA0676 | KIAA0676 protein | −0.83381 | 0.003755 |
| 201638_s_at | CPSF1 | cleavage and polyadenylation specific factor 1, 160 kDa | −0.83768 | 0.001261 |
| 217635_s_at | POLG | polymerase (DNA directed), gamma | −0.84235 | 0.00803 |
| 218062_x_at | CDC42EP4 | CDC42 effector protein (Rho GTPase binding) 4 | −0.84668 | 0.009831 |
| 52940_at | SIGIRR | single immunoglobulin and toll-interleukin 1 receptor (TIR) | −0.84946 | 0.00038 |
| | | domain | | |
| 206431_x_at | KIAA0676 | KIAA0676 protein | −0.8733 | 0.004129 |
| 216017_s_at | NAB2 | NGFI-A binding protein 2 (EGR1 binding protein 2) | −0.90116 | 0.002577 |
| 212155_at | RNF187 | ring finger protein 187 | −0.90251 | 0.004585 |
| 203733_at | DEXI | dexamethasone-induced transcript | −0.90678 | 0.00441 |
| 214564_s_at | PCDHGC3 | protocadherin gamma subfamily C, 3 | −0.9124 | 0.00484 |
| 213932_x_at | HLA-A | Major histocompatibility complex, class I, A | −0.91489 | 0.004634 |
| 209151_x_at | TCF3 | transcription factor 3 (E2A immunoglobulin enhancer | −0.9237 | 0.006165 |
| | | binding factors E12/E47) | | |
| 219777_at | GIMAP6 | GTPase, IMAP family member 6 | −0.92938 | 0.004519 |
| 200784_s_at | LRP1 | low density lipoprotein-related protein 1 (alpha-2- | −0.93157 | 0.002028 |
| | | macroglobulin receptor) | | |
| 218921_at | SIGIRR | single immunoglobulin and toll-interleukin 1 receptor (TIR) | −0.93915 | 0.000191 |
| | | domain | | |
| 218337_at | RAI16 | retinoic acid induced 16 | −0.94326 | 0.00886 |
| 218454_at | FLJ22662 | hypothetical protein FLJ22662 | −0.9525 | 0.001467 |
| 210449_x_at | MAPK14 | mitogen-activated protein kinase 14 | −0.96009 | 0.003314 |
| 210972_x_at | TRA@ /// TRDV2 | T cell receptor alpha locus /// T cell receptor delta variable | −0.96124 | 0.009068 |
| | /// TRAV20 /// | 2 /// T cell receptor alpha variable 20 /// T cell receptor | | |
| | TRAJ17 /// TRAC | alpha joining 17 /// T cell receptor alpha constant | | |
| 212613_at | BTN3A2 | butyrophilin, subfamily 3, member A2 | −0.966 | 0.003251 |
| 210594_x_at | MPZL1 | myelin protein zero-like 1 | −0.9726 | 0.001493 |
| 211656_x_at | HLA-DQB1 | major histocompatibility complex, class II, DQ beta 1 /// | −0.97359 | 0.008209 |
| | | major histocompatibility complex, class II, DQ beta 1 | | |
| 212969_x_at | EML3 | echinoderm microtubule associated protein like 3 | −0.97783 | 0.002431 |
| 217399_s_at | FOXO3A | forkhead box O3A | −0.98874 | 0.000866 |
| 38241_at | BTN3A3 | butyrophilin, subfamily 3, member A3 | −0.98912 | 0.005224 |
| 211779_x_at | AP2A2 | adaptor-related protein complex 2, alpha 2 subunit /// | −0.98928 | 0.002939 |
| | | adaptor-related protein complex 2, alpha 2 subunit | | |
| 204821_at | BTN3A3 | butyrophilin, subfamily 3, member A3 | −0.99177 | 0.00376 |
| 202108_at | PEPD | peptidase D | −0.9956 | 0.001981 |
| 205641_s_at | TRADD | TNFRSF1A-associated via death domain | −1.00386 | 0.005591 |
| 208452_x_at | MYO9B | myosin IXB | −1.00471 | 0.008675 |
| 1729_at | TRADD | TNFRSF1A-associated via death domain | −1.00623 | 0.003376 |
| 205582_s_at | GGTLA1 | gamma-glutamyltransferase-like activity 1 | −1.00714 | 0.005459 |
| 203417_at | MFAP2 | microfibrillar-associated protein 2 | −1.01063 | 0.004099 |
| 203567_s_at | TRIM38 | tripartite motif-containing 38 | −1.01518 | 0.00722 |
| 221807_s_at | PP2447 | hypothetical protein PP2447 | −1.01859 | 0.002653 |
| 212294_at | GNG12 | guanine nucleotide binding protein (G protein), gamma 12 | −1.02169 | 0.007683 |
| 213537_at | HLA-DPA1 | major histocompatibility complex, class II, DP alpha 1 | −1.02887 | 0.007775 |
| 205057_s_at | IDUA | iduronidase, alpha-L- | −1.02952 | 0.008204 |
| 202253_s_at | DNM2 | dynamin 2 | −1.0356 | 0.003634 |
| 204989_s_at | ITGB4 | integrin, beta 4 | −1.03812 | 0.005783 |
| 216587_s_at | FZD8 | frizzled homolog 8 (Drosophila) | −1.04165 | 0.000195 |
| 209038_s_at | EHD1 | EH-domain containing 1 | −1.07689 | 0.000435 |
| 200879_s_at | EPAS1 | endothelial PAS domain protein 1 | −1.07827 | 0.005357 |
| 208887_at | EIF3S4 | eukaryotic translation initiation factor 3, subunit 4 delta, | −1.07907 | 0.005199 |
| | | 44 kDa | | |
| 200754_x_at | SFRS2 | splicing factor, arginine/serine-rich 2 | −1.08153 | 0.001966 |
| 209166_s_at | MAN2B1 | mannosidase, alpha, class 2B, member 1 | −1.08936 | 0.009711 |
| 204715_at | PANX1 | pannexin 1 | −1.09622 | 0.002005 |
| 201271_s_at | RALY | RNA binding protein, autoantigenic (hnRNP-associated | −1.10474 | 0.008964 |
| | | with lethal yellow homolog (mouse)) | | |
| 212201_at | KIAA0692 | KIAA0692 protein | −1.12509 | 0.008471 |
| 203300_x_at | AP1S2 | adaptor-related protein complex 1, sigma 2 subunit | −1.13007 | 0.000427 |
| 200753_x_at | SFRS2 | splicing factor, arginine/serine-rich 2 | −1.13784 | 0.001244 |
| 215489_x_at | HOMER3 | homer homolog 3 (Drosophila) | −1.15018 | 0.001966 |
| 211065_x_at | PFKL | phosphofructokinase, liver /// phosphofructokinase, liver | −1.16035 | 0.003765 |
| 200704_at | LITAF | lipopolysaccharide-induced TNF factor | −1.16415 | 0.006365 |
| 210778_s_at | MXD4 | MAX dimerization protein 4 | −1.16878 | 0.001405 |
| 203749_s_at | RARA | retinoic acid receptor, alpha | −1.17184 | 0.002959 |
| 203669_s_at | DGAT1 | diacylglycerol O-acyltransferase homolog 1 (mouse) | −1.17387 | 0.00441 |
| 213072_at | CYHR1 | cysteine/histidine-rich 1 | −1.18141 | 0.00089 |
| 214459_x_at | HLA-C | major histocompatibility complex, class I, C | −1.19253 | 0.008783 |
| 1405_i_at | CCL5 | chemokine (C-C motif) ligand 5 | −1.19347 | 0.002527 |
| 213716_s_at | SECTM1 | secreted and transmembrane 1 | −1.19552 | 0.000349 |
| 201309_x_at | C5orf13 | chromosome 5 open reading frame 13 | −1.19692 | 0.002882 |
| 210930_s_at | ERBB2 | v-erb-b2 erythroblastic leukemia viral oncogene homolog | −1.20882 | 0.004858 |
| | | 2, neuro/glioblastoma derived oncogene homolog (avian) | | |
| 215116_s_at | DNM1 | dynamin 1 | −1.21115 | 0.000173 |
| 210269_s_at | DXYS155E | DNA segment on chromosome X and Y (unique) 155 | −1.21645 | 0.00788 |
| | | expressed sequence | | |
| 219575_s_at | PDF /// COG8 | peptide deformylase-like protein /// component of | −1.22854 | 0.002583 |
| | | oligomeric golgi complex 8 | | |
| 200706_s_at | LITAF | lipopolysaccharide-induced TNF factor | −1.23478 | 0.008528 |
| 221785_at | WIZ | widely-interspaced zinc finger motifs | −1.23555 | 0.003656 |
| 201137_s_at | HLA-DPB1 | major histocompatibility complex, class II, DP beta 1 | −1.26192 | 0.005489 |
| 213901_x_at | RBM9 | RNA binding motif protein 9 | −1.26856 | 0.004455 |
| 210973_s_at | FGFR1 | fibroblast growth factor receptor 1 (fms-related tyrosine | −1.27192 | 0.000627 |
| | | kinase 2, Pfeiffer syndrome) | | |
| 220189_s_at | MGAT4B | mannosyl (alpha-1,3-)-glycoprotein beta-1,4-N- | −1.27367 | 0.007236 |
| | | acetylglucosaminyltransferase, isoenzyme B | | |
| 209083_at | CORO1A | coronin, actin binding protein, 1A | −1.27403 | 0.00115 |
| 203274_at | F8A1 | coagulation factor VIII-associated (intronic transcript) 1 | −1.28233 | 0.00521 |
| 210757_x_at | DAB2 | disabled homolog 2, mitogen-responsive phosphoprotein | −1.28618 | 0.002714 |
| | | (Drosophila) | | |
| 203005_at | LTBR | lymphotoxin beta receptor (TNFR superfamily, member 3) | −1.29124 | 0.006501 |
| 203771_s_at | BLVRA | biliverdin reductase A | −1.29211 | 0.001352 |
| 212829_at | — | CDNA FLJ13267 fis, clone OVARC1000964 /// CDNA | −1.32372 | 0.002998 |
| | | FLJ13267 fis, clone OVARC1000964 | | |
| 212764_at | — | — | −1.32423 | 0.006375 |
| 208306_x_at | HLA-DRB5 | Major histocompatibility complex, class II, DR beta 3 | −1.32972 | 0.004616 |
| 201609_x_at | ICMT | isoprenylcysteine carboxyl methyltransferase | −1.33083 | 0.001386 |
| 204938_s_at | PLN | phospholamban | −1.34398 | 0.005261 |
| 217430_x_at | COL1A1 | collagen, type I, alpha 1 | −1.35597 | 0.006694 |
| 210202_s_at | BIN1 | bridging integrator 1 | −1.36271 | 0.009731 |
| 217757_at | A2M | alpha-2-macroglobulin | −1.36418 | 0.006867 |
| 209129_at | TRIP6 | thyroid hormone receptor interactor 6 | −1.36573 | 0.0087 |
| 210974_s_at | AP3D1 | adaptor-related protein complex 3, delta 1 subunit | −1.3677 | 0.000717 |
| 209350_s_at | GPS2 | G protein pathway suppressor 2 | −1.37152 | 0.001217 |
| 217912_at | DUS1L | dihydrouridine synthase 1-like (S. cerevisiae) | −1.39203 | 0.00833 |
| 204820_s_at | BTN3A3 /// | butyrophilin, subfamily 3, member A3 /// butyrophilin, | −1.40039 | 0.000145 |
| | BTN3A2 | subfamily 3, member A2 | | |
| 216603_at | SLC7A8 | Solute carrier family 7 (cationic amino acid transporter, y+ | −1.40095 | 0.007789 |
| | | system), member 8 | | |
| 201809_s_at | ENG | endoglin (Osler-Rendu-Weber syndrome 1) | −1.40133 | 0.004058 |
| 209356_x_at | EFEMP2 | EGF-containing fibulin-like extracellular matrix protein 2 | −1.40444 | 7.15E−05 |
| 213125_at | OLFML2B | olfactomedin-like 2B | −1.40665 | 0.008364 |
| 204655_at | CCL5 | chemokine (C-C motif) ligand 5 /// chemokine (C-C motif) | −1.41262 | 0.004745 |
| | | ligand 5 | | |
| 204941_s_at | ALDH3B2 | aldehyde dehydrogenase 3 family, member B2 | −1.41969 | 0.006283 |
| 202086_at | MX1 | myxovirus (influenza virus) resistance 1, interferon- | −1.42531 | 0.004155 |
| | | inducible protein p78 (mouse) /// myxovirus (influenza | | |
| | | virus) resistance 1, interferon-inducible protein p78 | | |
| | | (mouse) | | |
| 201279_s_at | DAB2 | disabled homolog 2, mitogen-responsive phosphoprotein | −1.42604 | 0.0003 |
| | | (Drosophila) | | |
| 208270_s_at | RNPEP | arginyl aminopeptidase (aminopeptidase B) | −1.4374 | 0.00073 |
| 202424_at | MAP2K2 | mitogen-activated protein kinase kinase 2 | −1.44086 | 0.006954 |
| 216526_x_at | HLA-C | major histocompatibility complex, class I, C | −1.44302 | 0.00023 |
| 207826_s_at | ID3 | inhibitor of DNA binding 3, dominant negative helix-loop- | −1.44509 | 0.009601 |
| | | helix protein | | |
| 217762_s_at | RAB31 | RAB31, member RAS oncogene family | −1.44782 | 0.008251 |
| 200752_s_at | CAPN1 | calpain 1, (mu/I) large subunit | −1.46225 | 0.002717 |
| 213193_x_at | TRBV19 /// | T cell receptor beta variable 19 /// T cell receptor beta | −1.46489 | 0.001138 |
| | TRBC1 | variable 19 /// T cell receptor beta constant 1 /// T cell | | |
| | | receptor beta constant 1 | | |
| 212025_s_at | FLII | flightless I homolog (Drosophila) | −1.46962 | 0.005018 |
| 212646_at | RAFTLIN | raft-linking protein | −1.47769 | 0.004248 |
| 208947_s_at | RENT1 | regulator of nonsense transcripts 1 | −1.48065 | 0.001272 |
| 210892_s_at | GTF2I | general transcription factor II, i | −1.49638 | 0.005113 |
| 202326_at | EHMT2 | euchromatic histone-lysine N-methyltransferase 2 | −1.49951 | 0.004361 |
| 209264_s_at | TSPAN4 | tetraspanin 4 | −1.50061 | 0.003844 |
| 201102_s_at | PFKL | phosphofructokinase, liver | −1.50118 | 0.000642 |
| 212940_at | COL6A1 | collagen, type VI, alpha 1 | −1.53744 | 0.008081 |
| 211796_s_at | TRBV21-1 /// | T cell receptor beta variable 21-1 /// T cell receptor beta | −1.54546 | 0.004773 |
| | TRBV19 /// | variable 19 /// T cell receptor beta variable 5-4 /// T cell | | |
| | TRBV5-4 /// | receptor beta variable 3-1 /// T cell receptor beta constant 1 | | |
| | TRBV3-1 /// | | | |
| | TRBC1 | | | |
| 205748_s_at | RNF126 | ring finger protein 126 | −1.54774 | 0.001837 |
| 213603_s_at | RAC2 | ras-related C3 botulinum toxin substrate 2 (rho family, | −1.57268 | 2.79E−05 |
| | | small GTP binding protein Rac2) | | |
| 221267_s_at | C19orf27 | chromosome 19 open reading frame 27 /// chromosome | −1.58204 | 2.26E−05 |
| | | 19 open reading frame 27 | | |
| 212687_at | LIMS1 | LIM and senescent cell antigen-like domains 1 | −1.58394 | 0.001718 |
| 205462_s_at | HPCAL1 | hippocalcin-like 1 | −1.59498 | 0.000239 |
| 214022_s_at | IFITM1 | interferon induced transmembrane protein 1 (9-27) | −1.60767 | 0.005327 |
| 203758_at | CTSO | cathepsin O | −1.60908 | 0.00246 |
| 217729_s_at | AES | amino-terminal enhancer of split | −1.61466 | 0.000731 |
| 209034_at | PNRC1 | proline-rich nuclear receptor coactivator 1 | −1.61945 | 0.001488 |
| 205683_x_at | TPSAB1 | tryptase alpha/beta 1 | −1.62549 | 0.005441 |
| 218559_s_at | MAFB | v-maf musculoaponeurotic fibrosarcoma oncogene | −1.62729 | 0.000277 |
| | | homolog B (avian) | | |
| 217456_x_at | HLA-E | major histocompatibility complex, class I, E | −1.63437 | 0.00014 |
| 212154_at | SDC2 | syndecan 2 (heparan sulfate proteoglycan 1, cell surface- | −1.64398 | 0.004244 |
| | | associated, fibroglycan) | | |
| 220266_s_at | KLF4 | Kruppel-like factor 4 (gut) | −1.65117 | 0.009049 |
| 210084_x_at | TPSAB1 | tryptase alpha/beta 1 | −1.65813 | 0.008036 |
| 201278_at | DAB2 | Disabled homolog 2, mitogen-responsive phosphoprotein | −1.6602 | 7.45E−05 |
| | | (Drosophila) | | |
| 217823_s_at | UBE2J1 | ubiquitin-conjugating enzyme E2, J1 (UBC6 homolog, | −1.67896 | 0.005511 |
| | | yeast) | | |
| 201508_at | IGFBP4 | insulin-like growth factor binding protein 4 | −1.68522 | 0.002388 |
| 202803_s_at | ITGB2 | integrin, beta 2 (antigen CD18 (p95), lymphocyte function- | −1.69425 | 0.00015 |
| | | associated antigen 1; macrophage antigen 1 (mac-1) beta | | |
| | | subunit) | | |
| 221972_s_at | SDF4 | stromal cell derived factor 4 | −1.70034 | 0.000312 |
| 204452_s_at | FZD1 | frizzled homolog 1 (Drosophila) | −1.70124 | 0.000198 |
| 221875_x_at | HLA-F | major histocompatibility complex, class I, F | −1.70772 | 0.004049 |
| 201641_at | BST2 | bone marrow stromal cell antigen 2 | −1.7259 | 0.002772 |
| 202269_x_at | GBP1 | guanylate binding protein 1, interferon-inducible, 67 kDa /// | −1.72769 | 0.003229 |
| | | guanylate binding protein 1, interferon-inducible, 67 kDa | | |
| 213290_at | COL6A2 | collagen, type VI, alpha 2 | −1.72876 | 3.96E−05 |
| 216594_x_at | AKR1C1 | aldo-keto reductase family 1, member C1 (dihydrodiol | −1.73395 | 0.00763 |
| | | dehydrogenase 1; 20-alpha (3-alpha)-hydroxysteroid | | |
| | | dehydrogenase) | | |
| 215313_x_at | HLA-A | major histocompatibility complex, class I, A | −1.7586 | 0.000347 |
| 217897_at | FXYD6 | FXYD domain containing ion transport regulator 6 | −1.75875 | 0.004604 |
| 205315_s_at | SNTB2 | syntrophin, beta 2 (dystrophin-associated protein A1, | −1.75932 | 0.00632 |
| | | 59 kDa, basic component 2) | | |
| 209261_s_at | NR2F6 | nuclear receptor subfamily 2, group F, member 6 | −1.76202 | 5.32E−05 |
| 204135_at | DOC1 | downregulated in ovarian cancer 1 | −1.78565 | 0.007502 |
| 201315_x_at | IFITM2 | interferon induced transmembrane protein 2 (1-8D) | −1.7927 | 0.006411 |
| 215923_s_at | PSD4 | pleckstrin and Sec7 domain containing 4 | −1.79879 | 0.007615 |
| 208966_x_at | IFI16 | interferon, gamma-inducible protein 16 | −1.80351 | 0.0082 |
| 212575_at | C19orf6 | chromosome 19 open reading frame 6 | −1.81288 | 5.94E−05 |
| 215464_s_at | TAX1BP3 | Tax1 (human T-cell leukemia virus type I) binding protein 3 | −1.81314 | 3.61E−05 |
| 216474_x_at | TPSAB1 /// | tryptase alpha/beta 1 /// tryptase beta 2 | −1.81811 | 0.004967 |
| | TPSB2 | | | |
| 208637_x_at | ACTN1 | actinin, alpha 1 | −1.82179 | 0.005568 |
| 201540_at | FHL1 | four and a half LIM domains 1 | −1.82184 | 0.008282 |
| 201727_s_at | ELAVL1 | ELAV (embryonic lethal, abnormal vision, Drosophila)-like | −1.84045 | 0.002956 |
| | | 1 (Hu antigen R) | | |
| 213293_s_at | TRIM22 | tripartite motif-containing 22 | −1.85487 | 0.000663 |
| 212671_s_at | HLA-DQA1 /// | major histocompatibility complex, class II, DQ alpha 1 /// | −1.85494 | 0.005757 |
| | HLA-DQA2 | major histocompatibility complex, class II, DQ alpha 2 | | |
| 200795_at | SPARCL1 | SPARC-like 1 (mast9, hevin) | −1.88146 | 0.001011 |
| 201601_x_at | IFITM1 | interferon induced transmembrane protein 1 (9-27) | −1.8845 | 0.002461 |
| 204806_x_at | HLA-F | major histocompatibility complex, class I, F | −1.8901 | 0.009947 |
| 201149_s_at | TIMP3 | TIMP metallopeptidase inhibitor 3 (Sorsby fundus | −1.8904 | 0.001284 |
| | | dystrophy, pseudoinflammatory) | | |
| 210072_at | CCL19 | chemokine (C-C motif) ligand 19 | −1.89355 | 0.004993 |
| 208998_at | UCP2 | uncoupling protein 2 (mitochondrial, proton carrier) | −1.9024 | 0.001612 |
| 201401_s_at | ADRBK1 | adrenergic, beta, receptor kinase 1 | −1.90273 | 0.00701 |
| 213422_s_at | MXRA8 | matrix-remodelling associated 8 | −1.91604 | 8.71E−05 |
| 210915_x_at | TRBV19 /// | T cell receptor beta variable 19 /// T cell receptor beta | −1.92403 | 0.000631 |
| | TRBC1 | constant 1 | | |
| 201666_at | TIMP1 | TIMP metallopeptidase inhibitor 1 | −1.92682 | 0.004328 |
| 207788_s_at | SORBS3 | sorbin and SH3 domain containing 3 | −1.93591 | 0.003418 |
| 202994_s_at | FBLN1 | fibulin 1 | −1.93867 | 0.007311 |
| 204151_x_at | AKR1C1 | aldo-keto reductase family 1, member C1 (dihydrodiol | −1.95545 | 0.000524 |
| | | dehydrogenase 1; 20-alpha (3-alpha)-hydroxysteroid | | |
| | | dehydrogenase) | | |
| 203942_s_at | MARK2 | MAP/microtubule affinity-regulating kinase 2 | −1.96174 | 7.52E−05 |
| 208747_s_at | C1S | complement component 1, s subcomponent | −1.97406 | 0.000158 |
| 207808_s_at | PROS1 | protein S (alpha) | −1.97435 | 0.00286 |
| 211991_s_at | HLA-DPA1 | major histocompatibility complex, class II, DP alpha 1 | −1.99289 | 0.00046 |
| 212067_s_at | C1R | complement component 1, r subcomponent | −2.00528 | 0.000954 |
| 219607_s_at | MS4A4A | membrane-spanning 4-domains, subfamily A, member 4 | −2.00653 | 0.000276 |
| 211990_at | HLA-DPA1 | major histocompatibility complex, class II, DP alpha 1 | −2.00996 | 0.000895 |
| 218162_at | OLFML3 | olfactomedin-like 3 | −2.01228 | 0.000241 |
| 214247_s_at | DKK3 | dickkopf homolog 3 (Xenopus laevis) | −2.01752 | 0.008837 |
| 200796_s_at | MCL1 | myeloid cell leukemia sequence 1 (BCL2-related) | −2.02536 | 0.000545 |
| 212423_at | C10orf56 | chromosome 10 open reading frame 56 | −2.05422 | 0.008811 |
| 218718_at | PDGFC | platelet derived growth factor C | −2.05444 | 0.007694 |
| 201670_s_at | MARCKS | myristoylated alanine-rich protein kinase C substrate | −2.05799 | 0.002113 |
| 217478_s_at | HLA-DMA | major histocompatibility complex, class II, DM alpha | −2.07162 | 0.00266 |
| 200696_s_at | GSN | gelsolin (amyloidosis, Finnish type) | −2.0889 | 0.001634 |
| 212937_s_at | COL6A1 | collagen, type VI, alpha 1 | −2.10637 | 0.000273 |
| 203964_at | NMI | N-myc (and STAT) interactor | −2.11187 | 0.007323 |
| 210130_s_at | TM7SF2 | transmembrane 7 superfamily member 2 | −2.11234 | 2.97E−06 |
| 203766_s_at | LMOD1 | leiomodin 1 (smooth muscle) | −2.1405 | 0.000436 |
| 211813_x_at | DCN | decorin | −2.14944 | 0.000876 |
| 207016_s_at | ALDH1A2 | aldehyde dehydrogenase 1 family, member A2 | −2.15142 | 0.000544 |
| 203131_at | PDGFRA | platelet-derived growth factor receptor, alpha polypeptide | −2.15729 | 0.000843 |
| 219295_s_at | PCOLCE2 | procollagen C-endopeptidase enhancer 2 | −2.15942 | 0.001554 |
| 202283_at | SERPINF1 | serpin peptidase inhibitor, clade F (alpha-2 antiplasmin, | −2.16096 | 3.69E−05 |
| | | pigment epithelium derived factor), member 1 | | |
| 209209_s_at | PLEKHC1 | pleckstrin homology domain containing, family C (with | −2.16279 | 0.008575 |
| | | FERM domain) member 1 | | |
| 201044_x_at | DUSP1 | dual specificity phosphatase 1 | −2.17605 | 0.003233 |
| 202177_at | GAS6 | growth arrest-specific 6 | −2.17954 | 0.001073 |
| 201147_s_at | TIMP3 | TIMP metallopeptidase inhibitor 3 (Sorsby fundus | −2.19114 | 0.001147 |
| | | dystrophy, pseudoinflammatory) | | |
| 217767_at | C3 | complement component 3 | −2.20807 | 0.000409 |
| 204875_s_at | GMDS | GDP-mannose 4,6-dehydratase | −2.21051 | 0.001802 |
| 203851_at | IGFBP6 | insulin-like growth factor binding protein 6 | −2.22037 | 8.04E−05 |
| 209487_at | RBPMS | RNA binding protein with multiple splicing | −2.22901 | 0.006667 |
| 200974_at | ACTA2 | actin, alpha 2, smooth muscle, aorta | −2.2617 | 0.00324 |
| 208790_s_at | PTRF | polymerase I and transcript release factor | −2.26239 | 0.00114 |
| 201311_s_at | SH3BGRL | SH3 domain binding glutamic acid-rich protein like | −2.26733 | 0.008612 |
| 201280_s_at | DAB2 | disabled homolog 2, mitogen-responsive phosphoprotein | −2.26916 | 0.00016 |
| | | (Drosophila) | | |
| 211864_s_at | FER1L3 | fer-1-like 3, myoferlin (C. elegans) | −2.30992 | 0.009752 |
| 214505_s_at | FHL1 | four and a half LIM domains 1 | −2.31098 | 0.002028 |
| 209113_s_at | HMG20B | high-mobility group 20B | −2.32179 | 0.003738 |
| 201852_x_at | COL3A1 | collagen, type III, alpha 1 (Ehlers-Danlos syndrome type | −2.32476 | 0.006918 |
| | | IV, autosomal dominant) | | |
| 208944_at | TGFBR2 | transforming growth factor, beta receptor II (70/80 kDa) | −2.37037 | 0.008003 |
| 201893_x_at | DCN | decorin | −2.37312 | 0.000542 |
| 212091_s_at | COL6A1 | collagen, type VI, alpha 1 | −2.39348 | 9.55E−06 |
| 213428_s_at | COL6A1 | collagen, type VI, alpha 1 | −2.40702 | 0.001384 |
| 219922_s_at | LTBP3 | latent transforming growth factor beta binding protein 3 | −2.44169 | 0.00145 |
| 209286_at | CDC42EP3 | CDC42 effector protein (Rho GTPase binding) 3 | −2.44278 | 0.009432 |
| 204442_x_at | LTBP4 | latent transforming growth factor beta binding protein 4 | −2.44389 | 0.00061 |
| 204942_s_at | ALDH3B2 | aldehyde dehydrogenase 3 family, member B2 | −2.44641 | 0.006218 |
| 201668_x_at | MARCKS | myristoylated alanine-rich protein kinase C substrate | −2.45345 | 0.000485 |
| 200907_s_at | KIAA0992 | palladin | −2.47332 | 0.007584 |
| 204163_at | EMILIN1 | elastin microfibril interfacer 1 | −2.48498 | 0.001723 |
| 202409_at | LOC492304 | putative insulin-like growth factor II associated protein | −2.53724 | 0.002963 |
| 203085_s_at | TGFB1 | transforming growth factor, beta 1 (Camurati-Engelmann | −2.54778 | 3.26E−06 |
| | | disease) | | |
| 212016_s_at | PTBP1 | polypyrimidine tract binding protein 1 | −2.55013 | 0.000697 |
| 204931_at | TCF21 | transcription factor 21 | −2.56319 | 0.001324 |
| 200904_at | HLA-E | major histocompatibility complex, class I, E | −2.57234 | 2.07E−05 |
| 207961_x_at | MYH11 | myosin, heavy polypeptide 11, smooth muscle | −2.57354 | 0.004572 |
| 209619_at | CD74 | CD74 antigen (invariant polypeptide of major | −2.58672 | 0.008069 |
| | | histocompatibility complex, class II antigen-associated) | | |
| 209488_s_at | RBPMS | RNA binding protein with multiple splicing | −2.60676 | 0.003118 |
| 210299_s_at | FHL1 | four and a half LIM domains 1 | −2.61111 | 0.006014 |
| 203890_s_at | DAPK3 | death-associated protein kinase 3 | −2.62193 | 0.005771 |
| 202222_s_at | DES | desmin | −2.6422 | 0.008325 |
| 201787_at | FBLN1 | fibulin 1 | −2.65593 | 0.000172 |
| 221667_s_at | HSPB8 | heat shock 22 kDa protein 8 | −2.72126 | 0.004204 |
| 201539_s_at | FHL1 | four and a half LIM domains 1 | −2.77375 | 0.005409 |
| 209651_at | TGFB1I1 | transforming growth factor beta 1 induced transcript 1 | −2.80218 | 0.007543 |
| 206391_at | RARRES1 | retinoic acid receptor responder (tazarotene induced) 1 | −2.8163 | 0.000645 |
| 201551_s_at | LAMP1 | lysosomal-associated membrane protein 1 | −2.81852 | 0.005412 |
| 203453_at | SCNN1A | sodium channel, nonvoltage-gated 1 alpha | −2.90666 | 0.002618 |
| 201497_x_at | MYH11 | myosin, heavy polypeptide 11, smooth muscle | −2.98474 | 0.008825 |
| 221872_at | RARRES1 | retinoic acid receptor responder (tazarotene induced) 1 | −3.05437 | 0.001816 |
| 200986_at | SERPING1 | serpin peptidase inhibitor, clade G (C1 inhibitor), member | −3.0594 | 2.65E−05 |
| | | 1, (angioedema, hereditary) | | |
| 212574_x_at | C19orf6 | chromosome 19 open reading frame 6 | −3.13178 | 1.84E−05 |
| 200923_at | LGALS3BP | lectin, galactoside-binding, soluble, 3 binding protein | −3.18309 | 5.59E−06 |
| 214040_s_at | GSN | gelsolin (amyloidosis, Finnish type) | −3.18941 | 4.71E−07 |
| 213986_s_at | C19orf6 | chromosome 19 open reading frame 6 | −3.19533 | 6.08E−06 |
| 210298_x_at | FHL1 | four and a half LIM domains 1 | −3.2298 | 0.002585 |
| 202995_s_at | FBLN1 | fibulin 1 | −3.23923 | 7.20E−06 |
| 214251_s_at | NUMA1 | nuclear mitotic apparatus protein 1 | −3.31103 | 0.000616 |
| 214752_x_at | FLNA | filamin A, alpha (actin binding protein 280) | −3.33076 | 0.002287 |
| 206392_s_at | RARRES1 | retinoic acid receptor responder (tazarotene induced) 1 | −3.42531 | 0.0001 |
| 216971_s_at | PLEC1 | plectin 1, intermediate filament binding protein 500 kDa | −3.54374 | 2.04E−05 |
| 211564_s_at | PDLIM4 | PDZ and LIM domain 4 | −3.58703 | 0.000624 |
| 200859_x_at | FLNA | filamin A, alpha (actin binding protein 280) | −3.61765 | 0.000526 |
| 209156_s_at | COL6A2 | collagen, type VI, alpha 2 | −3.65904 | 0.002408 |
| 213176_s_at | LTBP4 | latent transforming growth factor beta binding protein 4 | −3.83594 | 1.59E−07 |
| 213746_s_at | FLNA | filamin A, alpha (actin binding protein 280) | −4.20067 | 0.001409 |

TABLE 37
dataset 2 (stroma)

| uniqueID | slopeS | probS |
|---|---|---|
| HLA-DPB1 | 1.098455644 | 0.008690666 |
| DKFZp761D221 | 0.934159835 | 0.007873718 |
| GI_22761402 | −0.815995448 | 0.005679887 |

TABLE 38
dataset 2 (tumor)

| uniqueID | slopeT | probT |
|---|---|---|
| GI_2094528 | 5.453123 | 7.29E−08 |
| GRP | 4.33782 | 1.81E−10 |
| CSMD1 | 3.474385 | 0.003859 |
| MKI67 | 2.539772 | 0.001274 |
| MMP9 | 2.213091 | 0.003619 |
| GI_22761402 | 2.036472 | 3.80E−10 |
| GI_16550429 | 1.974206 | 5.21E−05 |
| NRG1 | 1.586801 | 0.008504 |
| CSPG2 | 1.546295 | 0.00394 |
| ATP6V1E2 | 1.478015 | 0.007254 |
| SALL4 | 1.477018 | 0.002289 |
| KHDRBS3 | 1.444605 | 0.001988 |
| CDKN2A | 1.350577 | 0.009437 |
| WISP1 | 1.146656 | 0.000726 |
| KIAA0389 | 0.91715 | 0.006525 |
| MNAT1 | 0.912261 | 0.007793 |
| ARFIP2 | 0.876391 | 0.00014 |
| LAMA4 | 0.864271 | 0.003849 |
| BAX | 0.841154 | 0.007583 |
| AML1 | 0.810606 | 0.006099 |
| ITGA5 | 0.780658 | 0.009293 |
| CTBP1 | 0.678406 | 0.009205 |
| KIAA1109 | 0.67286 | 0.007531 |
| MAP2K1IP1 | −0.53796 | 0.007869 |
| G2AN | −0.58591 | 0.006474 |
| Prostein | −0.83684 | 0.008957 |
| AZGP1 | −1.16268 | 0.000864 |
| DKFZp586J0119 | −1.1717 | 0.002842 |
| HLA-DPB1 | −1.39602 | 0.002492 |
| DLG2 | −1.70437 | 0.000225 |
| PART1 | −2.25762 | 0.000612 |
| MCCC2 | −2.27693 | 1.48E−05 |
| GDEP | −2.88929 | 0.000495 |
| CCK | −3.66888 | 4.84E−05 |
| GABRG2 | −5.02487 | 0.000587 |

TABLE 39
dataset 3 (stroma)

| | | | log base | |
|---|---|---|---|---|
| | | | 2 fold | |
| uniqueID | Gene Symbol | Gene Title | change | p value |
| 203413_at | NELL2 | NEL-like 2 (chicken) /// NEL-like 2 (chicken) | 5.489204 | 1.80E−06 |
| 218835_at | SFTPA2 | surfactant, pulmonary-associated protein A2 | 4.99448 | 0.000853 |
| 213068_at | DPT | dermatopontin | 4.941844 | 8.05E−10 |
| 213791_at | PENK | proenkephalin | 4.627489 | 1.54E−22 |
| 202409_at | LOC492304 | putative insulin-like growth factor II associated protein | 4.301564 | 7.02E−25 |
| 212187_x_at | PTGDS | prostaglandin D2 synthase 21 kDa (brain) | 4.282127 | 4.81E−08 |
| 211663_x_at | PTGDS | prostaglandin D2 synthase 21 kDa (brain) /// prostaglandin D2 | 4.162189 | 3.70E−11 |
| | | synthase 21 kDa (brain) | | |
| 206211_at | SELE | selectin E (endothelial adhesion molecule 1) | 4.141967 | 0.000374 |
| 219230_at | FLJ10970 | hypothetical protein FLJ10970 | 4.136925 | 2.21E−06 |
| 207977_s_at | DPT | dermatopontin | 4.006443 | 8.87E−07 |
| 205431_s_at | BMP5 | bone morphogenetic protein 5 | 3.951413 | 4.18E−08 |
| 211748_x_at | PTGDS | prostaglandin D2 synthase 21 kDa (brain) /// prostaglandin D2 | 3.934415 | 1.50E−07 |
| | | synthase 21 kDa (brain) | | |
| 202746_at | ITM2A | integral membrane protein 2A | 3.796333 | 5.80E−08 |
| 221491_x_at | HLA-DRB1 | major histocompatibility complex, class II, DR beta 1 /// major | 3.790244 | 1.91E−08 |
| | | histocompatibility complex, class II, DR beta 1 | | |
| 208727_s_at | CDC42 | cell division cycle 42 (GTP binding protein, 25 kDa) | 3.757378 | 0.001057 |
| 209170_s_at | GPM6B | glycoprotein M6B | 3.755294 | 2.75E−16 |
| 211734_s_at | FCER1A | Fc fragment of IgE, high affinity I, receptor for; alpha polypeptide | 3.664566 | 5.87E−12 |
| | | /// Fc fragment of IgE, high affinity I, receptor for; alpha | | |
| | | polypeptide | | |
| 203382_s_at | APOE | apolipoprotein E | 3.63542 | 1.20E−06 |
| 205200_at | CLEC3B | C-type lectin domain family 3, member B | 3.629254 | 9.42E−25 |
| 205404_at | HSD11B1 | hydroxysteroid (11-beta) dehydrogenase 1 | 3.571465 | 1.15E−17 |
| 209167_at | GPM6B | glycoprotein M6B | 3.553804 | 4.51E−10 |
| 219759_at | LRAP | leukocyte-derived arginine aminopeptidase | 3.542274 | 4.11E−05 |
| 219266_at | ZNF350 | zinc finger protein 350 | 3.31832 | 0.003402 |
| 212999_x_at | HLA-DQB1 | Major histocompatibility complex, class II, DQ beta 1 /// Major | 3.309683 | 2.92E−11 |
| | | histocompatibility complex, class II, DQ beta 1 | | |
| 209466_x_at | PTN | pleiotrophin (heparin binding growth factor 8, neurite growth- | 3.296314 | 0.000107 |
| | | promoting factor 1) | | |
| 206049_at | SELP | selectin P (granule membrane protein 140 kDa, antigen CD62) | 3.293419 | 1.72E−24 |
| 219360_s_at | TRPM4 | transient receptor potential cation channel, subfamily M, member 4 | 3.232353 | 0.000393 |
| 213071_at | DPT | dermatopontin | 3.140798 | 2.80E−08 |
| 211737_x_at | PTN | pleiotrophin (heparin binding growth factor 8, neurite growth- | 3.135266 | 0.001864 |
| | | promoting factor 1) /// pleiotrophin (heparin binding growth factor | | |
| | | 8, neurite growth-promoting factor 1) | | |
| 209480_at | HLA-DQB1 | Major histocompatibility complex, class II, DQ beta 1 /// Major | 3.104849 | 1.14E−06 |
| | | histocompatibility complex, class II, DQ beta 1 | | |
| 203381_s_at | APOE | apolipoprotein E | 3.103849 | 0.000211 |
| 208335_s_at | FY | Duffy blood group | 3.05992 | 2.03E−06 |
| 58916_at | KCTD14 | potassium channel tetramerisation domain containing 14 | 3.017711 | 2.41E−08 |
| 209168_at | GPM6B | glycoprotein M6B | 3.017066 | 1.50E−15 |
| 221690_s_at | NALP2 | NACHT, leucine rich repeat and PYD containing 2 | 3.008828 | 0.001826 |
| 205392_s_at | CCL14 /// CCL15 | chemokine (C-C motif) ligand 14 /// chemokine (C-C motif) ligand | 2.90463 | 7.54E−08 |
| | | 15 | | |
| 220532_s_at | LR8 | LR8 protein | 2.890372 | 6.71E−08 |
| 201325_s_at | EMP1 | epithelial membrane protein 1 | 2.866127 | 0.000366 |
| 218922_s_at | LASS4 | LAG1 longevity assurance homolog 4 (S. cerevisiae) | 2.847613 | 4.92E−09 |
| 205207_at | IL6 | interleukin 6 (interferon, beta 2) | 2.837542 | 0.000239 |
| 205430_at | BMP5 | bone morphogenetic protein 5 | 2.825151 | 9.03E−05 |
| 205242_at | CXCL13 | chemokine (C—X—C motif) ligand 13 (B-cell chemoattractant) | 2.796628 | 2.07E−10 |
| 204602_at | DKK1 | dickkopf homolog 1 (Xenopus laevis) | 2.781827 | 0.007679 |
| 211654_x_at | HLA-DQB1 | major histocompatibility complex, class II, DQ beta 1 /// major | 2.780187 | 1.03E−06 |
| | | histocompatibility complex, class II, DQ beta 1 | | |
| 207430_s_at | MSMB | microseminoprotein, beta- | 2.759453 | 0.000438 |
| 209465_x_at | PTN | pleiotrophin (heparin binding growth factor 8, neurite growth- | 2.745541 | 0.004492 |
| | | promoting factor 1) | | |
| 205898_at | CX3CR1 | chemokine (C—X3—C motif) receptor 1 | 2.741153 | 0.000124 |
| 203868_s_at | VCAM1 | vascular cell adhesion molecule 1 | 2.684526 | 7.86E−06 |
| 212884_x_at | APOE | Apolipoprotein E | 2.662926 | 6.87E−07 |
| 210297_s_at | MSMB | microseminoprotein, beta- | 2.655622 | 9.22E−05 |
| 209747_at | TGFB3 | transforming growth factor, beta 3 | 2.634028 | 2.10E−08 |
| 206071_s_at | EPHA3 | EPH receptor A3 | 2.620419 | 3.86E−11 |
| 205882_x_at | ADD3 | adducin 3 (gamma) | 2.609889 | 2.58E−08 |
| 202747_s_at | ITM2A | integral membrane protein 2A | 2.604758 | 1.12E−07 |
| 213258_at | TFPI | Tissue factor pathway inhibitor (lipoprotein-associated | 2.580382 | 2.48E−08 |
| | | coagulation inhibitor) | | |
| 213395_at | MLC1 | megalencephalic leukoencephalopathy with subcortical cysts 1 | 2.572899 | 4.39E−10 |
| 203052_at | C2 | complement component 2 | 2.568309 | 3.99E−06 |
| 212998_x_at | HLA-DQB1 | major histocompatibility complex, class II, DQ beta 1 /// major | 2.534972 | 0.000877 |
| | | histocompatibility complex, class II, DQ beta 1 | | |
| 201324_at | EMP1 | epithelial membrane protein 1 | 2.530238 | 0.003386 |
| 201842_s_at | EFEMP1 | EGF-containing fibulin-like extracellular matrix protein 1 | 2.525356 | 0.001269 |
| 215145_s_at | CNTNAP2 | contactin associated protein-like 2 | 2.514392 | 0.003233 |
| 204545_at | PEX6 | peroxisomal biogenesis factor 6 | 2.49286 | 9.98E−06 |
| 220283_at | KIAA1822L | KIAA1822-like | 2.475417 | 1.19E−13 |
| 206682_at | CLEC10A | C-type lectin domain family 10, member A | 2.443806 | 7.31E−12 |
| 202269_x_at | GBP1 | guanylate binding protein 1, interferon-inducible, 67 kDa /// | 2.438516 | 3.33E−05 |
| | | guanylate binding protein 1, interferon-inducible, 67 kDa | | |
| 219867_at | CHODL | chondrolectin | 2.414547 | 7.74E−08 |
| 209443_at | SERPINA5 | serpin peptidase inhibitor, clade A (alpha-1 antiproteinase, | 2.410818 | 3.05E−12 |
| | | antitrypsin), member 5 | | |
| 204368_at | SLCO2A1 | solute carrier organic anion transporter family, member 2A1 | 2.39768 | 0.000189 |
| 222304_x_at | OR7E47P | olfactory receptor, family 7, subfamily E, member 47 pseudogene | 2.381455 | 0.000604 |
| 208894_at | HLA-DRA | major histocompatibility complex, class II, DR alpha /// major | 2.377035 | 7.05E−05 |
| | | histocompatibility complex, class II, DR alpha | | |
| 221698_s_at | CLEC7A | C-type lectin domain family 7, member A /// C-type lectin domain | 2.371779 | 2.31E−07 |
| | | family 7, member A | | |
| 209201_x_at | CXCR4 | chemokine (C—X—C motif) receptor 4 | 2.369772 | 0.002292 |
| 219140_s_at | RBP4 | retinol binding protein 4, plasma | 2.366078 | 3.36E−21 |
| 201034_at | ADD3 | adducin 3 (gamma) | 2.346457 | 1.49E−05 |
| 205326_at | RAMP3 | receptor (calcitonin) activity modifying protein 3 | 2.295126 | 1.88E−07 |
| 214761_at | ZNF423 | zinc finger protein 423 | 2.278218 | 5.59E−12 |
| 207118_s_at | MMP23B /// | matrix metallopeptidase 23B /// matrix metallopeptidase 23A | 2.266738 | 4.31E−15 |
| | MMP23A | | | |
| 215193_x_at | HLA-DRB1 | major histocompatibility complex, class II, DR beta 1 | 2.246954 | 7.79E−07 |
| 202112_at | VWF | von Willebrand factor | 2.23189 | 3.57E−05 |
| 216598_s_at | CCL2 | chemokine (C-C motif) ligand 2 | 2.226284 | 0.002007 |
| 204394_at | SLC43A1 | solute carrier family 43, member 1 | 2.225854 | 3.83E−06 |
| 205073_at | CYP2J2 | cytochrome P450, family 2, subfamily J, polypeptide 2 | 2.2213 | 0.008273 |
| 215116_s_at | DNM1 | dynamin 1 | 2.219382 | 3.06E−08 |
| 209573_s_at | C18orf1 | chromosome 18 open reading frame 1 | 2.216492 | 6.80E−14 |
| 205432_at | OVGP1 | oviductal glycoprotein 1, 120 kDa (mucin 9, oviductin) | 2.198928 | 6.02E−07 |
| 203186_s_at | S100A4 | S100 calcium binding protein A4 (calcium protein, calvasculin, | 2.185376 | 0.000729 |
| | | metastasin, murine placental homolog) | | |
| 214598_at | CLDN8 | claudin 8 | 2.182942 | 0.001672 |
| 209735_at | ABCG2 | ATP-binding cassette, sub-family G (WHITE), member 2 | 2.149332 | 5.36E−08 |
| 206030_at | ASPA | aspartoacylase (Canavan disease) | 2.145077 | 2.69E−05 |
| 220988_s_at | C1QTNF3 | C1q and tumor necrosis factor related protein 3 /// C1q and | 2.142632 | 0.000899 |
| | | tumor necrosis factor related protein 3 | | |
| 208814_at | HSPA4 | Heat shock 70 kDa protein 4 | 2.140177 | 0.000139 |
| 219269_at | FLJ21616 | hypothetical protein FLJ21616 | 2.137572 | 0.001848 |
| 209398_at | HIST1H1C | histone 1, H1c | 2.129817 | 0.001397 |
| 218345_at | HCA112 | hepatocellular carcinoma-associated antigen 112 | 2.128669 | 2.01E−07 |
| 213059_at | CREB3L1 | cAMP responsive element binding protein 3-like 1 | 2.113389 | 7.06E−06 |
| 201858_s_at | PRG1 | proteoglycan 1, secretory granule | 2.111824 | 0.00024 |
| 210982_s_at | HLA-DRA | major histocompatibility complex, class II, DR alpha | 2.098275 | 0.000226 |
| 205347_s_at | TMSL8 | thymosin-like 8 | 2.090751 | 0.002065 |
| 204670_x_at | HLA-DRB1 | major histocompatibility complex, class II, DR beta 1 | 2.080329 | 9.22E−07 |
| 211919_s_at | CXCR4 | chemokine (C—X—C motif) receptor 4 /// chemokine (C—X—C motif) | 2.078332 | 0.004165 |
| | | receptor 4 | | |
| 210889_s_at | FCGR2B | Fc fragment of IgG, low affinity IIb, receptor (CD32) | 2.069481 | 5.10E−07 |
| 204260_at | CHGB | chromogranin B (secretogranin 1) | 2.062107 | 6.67E−08 |
| 203104_at | CSF1R | colony stimulating factor 1 receptor, formerly McDonough feline | 2.058921 | 1.12E−05 |
| | | sarcoma viral (v-fms) oncogene homolog /// colony stimulating | | |
| | | factor 1 receptor, formerly McDonough feline sarcoma viral (v- | | |
| | | fms) oncogene homolog | | |
| 201752_s_at | ADD3 | adducin 3 (gamma) | 2.051831 | 1.04E−05 |
| 205316_at | SLC15A2 | Solute carrier family 15 (H+/peptide transporter), member 2 | 2.050784 | 0.006469 |
| 201666_at | TIMP1 | TIMP metallopeptidase inhibitor 1 | 2.047738 | 0.007282 |
| 202242_at | TSPAN7 | tetraspanin 7 | 2.045386 | 1.02E−06 |
| 209312_x_at | HLA-DRB1 | major histocompatibility complex, class II, DR beta 1 /// major | 2.030949 | 8.04E−06 |
| | | histocompatibility complex, class II, DR beta 1 | | |
| 208949_s_at | LGALS3 /// | lectin, galactoside-binding, soluble, 3 (galectin 3) /// galectin-3 | 2.014096 | 8.43E−05 |
| | GALIG | internal gene | | |
| 201843_s_at | EFEMP1 | EGF-containing fibulin-like extracellular matrix protein 1 | 2.012838 | 8.96E−05 |
| 204955_at | SRPX | sushi-repeat-containing protein, X-linked | 2.010724 | 0.001692 |
| 202643_s_at | TNFAIP3 | tumor necrosis factor, alpha-induced protein 3 | 2.010615 | 0.00946 |
| 219405_at | TRIM68 | tripartite motif-containing 68 | 2.006525 | 0.005106 |
| 212670_at | ELN | elastin (supravalvular aortic stenosis, Williams-Beuren | 2.005247 | 0.000189 |
| | | syndrome) | | |
| 201108_s_at | THBS1 | thrombospondin 1 | 2.000481 | 0.008471 |
| 201137_s_at | HLA-DPB1 | major histocompatibility complex, class II, DP beta 1 | 1.994721 | 4.66E−05 |
| 204037_at | EDG2 | endothelial differentiation, lysophosphatidic acid G-protein- | 1.987127 | 3.55E−07 |
| | | coupled receptor, 2 | | |
| 205648_at | WNT2 | wingless-type MMTV integration site family member 2 | 1.982491 | 1.03E−05 |
| 201753_s_at | ADD3 | adducin 3 (gamma) | 1.980488 | 0.001529 |
| 218574_s_at | LMCD1 | LIM and cysteine-rich domains 1 | 1.970663 | 2.97E−06 |
| 215059_at | — | MRNA; cDNA DKFZp564G112 (from clone DKFZp564G112) | 1.968959 | 1.37E−09 |
| 213601_at | SLIT1 | slit homolog 1 (Drosophila) | 1.968699 | 1.30E−05 |
| 205620_at | F10 | coagulation factor X | 1.948339 | 1.70E−09 |
| 204820_s_at | BTN3A3 /// | butyrophilin, subfamily 3, member A3 /// butyrophilin, subfamily 3, | 1.943927 | 0.002256 |
| | BTN3A2 | member A2 | | |
| 212310_at | KIAA0268 /// | C219-reactive peptide /// AAAP6077 /// similar to C219-reactive | 1.94176 | 0.000253 |
| | UNQ6077 /// | peptide | | |
| | LOC440751 | | | |
| 213418_at | HSPA6 | heat shock 70 kDa protein 6 (HSP70B′) | 1.937575 | 0.000133 |
| 203619_s_at | FAIM2 | Fas apoptotic inhibitory molecule 2 | 1.928405 | 8.84E−11 |
| 208983_s_at | PECAM1 | platelet/endothelial cell adhesion molecule (CD31 antigen) | 1.917568 | 0.000819 |
| 202992_at | C7 | complement component 7 | 1.909132 | 0.000337 |
| 202410_x_at | IGF2 | insulin-like growth factor 2 (somatomedin A) | 1.908886 | 3.76E−10 |
| 219602_s_at | FAM38B | family with sequence similarity 38, member B | 1.894541 | 2.52E−16 |
| 210978_s_at | TAGLN2 | transgelin 2 | 1.893889 | 0.000161 |
| 213519_s_at | LAMA2 | laminin, alpha 2 (merosin, congenital muscular dystrophy) | 1.89175 | 4.73E−05 |
| 213844_at | HOXA5 | homeo box A5 | 1.891152 | 0.001277 |
| 210508_s_at | KCNQ2 | potassium voltage-gated channel, KQT-like subfamily, member 2 | 1.88277 | 5.65E−09 |
| 221087_s_at | APOL3 | apolipoprotein L, 3 | 1.880867 | 0.000279 |
| 320_at | PEX6 | peroxisomal biogenesis factor 6 | 1.878554 | 9.65E−05 |
| 205759_s_at | SULT2B1 | sulfotransferase family, cytosolic, 2B, member 1 | 1.875884 | 0.000175 |
| 219436_s_at | EMCN | endomucin | 1.869907 | 9.20E−08 |
| 209652_s_at | PGF | placental growth factor, vascular endothelial growth factor- | 1.861116 | 4.35E−11 |
| | | related protein | | |
| 219729_at | PRRX2 | paired related homeobox 2 | 1.853068 | 1.75E−06 |
| 205978_at | KL | klotho | 1.846235 | 0.001963 |
| 211603_s_at | ETV4 | ets variant gene 4 (E1A enhancer binding protein, E1AF) /// ets | 1.840957 | 3.24E−09 |
| | | variant gene 4 (E1A enhancer binding protein, E1AF) | | |
| 218084_x_at | FXYD5 | FXYD domain containing ion transport regulator 5 | 1.839466 | 1.14E−07 |
| 203886_s_at | FBLN2 | fibulin 2 | 1.831612 | 0.000219 |
| 219991_at | SLC2A9 | solute carrier family 2 (facilitated glucose transporter), member 9 | 1.828705 | 7.10E−11 |
| 221944_at | LOC283874 | Hypothetical protein LOC283874 | 1.823408 | 1.33E−09 |
| 204103_at | CCL4 | chemokine (C-C motif) ligand 4 | 1.822669 | 0.000153 |
| 221529_s_at | PLVAP | plasmalemma vesicle associated protein | 1.820398 | 0.00012 |
| 219747_at | FLJ23191 | hypothetical protein FLJ23191 | 1.818384 | 0.000831 |
| 205608_s_at | ANGPT1 | angiopoietin 1 | 1.815849 | 3.89E−06 |
| 210002_at | GATA6 | GATA binding protein 6 | 1.811896 | 0.004116 |
| 216114_at | NCKIPSD | NCK interacting protein with SH3 domain | 1.79883 | 1.44E−10 |
| 211991_s_at | HLA-DPA1 | major histocompatibility complex, class II, DP alpha 1 | 1.798669 | 0.001184 |
| 203979_at | CYP27A1 | cytochrome P450, family 27, subfamily A, polypeptide 1 | 1.784908 | 0.000374 |
| 217478_s_at | HLA-DMA | major histocompatibility complex, class II, DM alpha | 1.779529 | 0.00548 |
| 209619_at | CD74 | CD74 antigen (invariant polypeptide of major histocompatibility | 1.779286 | 1.70E−05 |
| | | complex, class II antigen-associated) | | |
| 204311_at | ATP1B2 | ATPase, Na+/K+ transporting, beta 2 polypeptide | 1.778801 | 5.05E−21 |
| 209823_x_at | HLA-DQB1 | major histocompatibility complex, class II, DQ beta 1 | 1.760789 | 0.009111 |
| 205818_at | DBC1 | deleted in bladder cancer 1 | 1.759038 | 0.000258 |
| 205779_at | RAMP2 | receptor (calcitonin) activity modifying protein 2 | 1.758379 | 1.58E−07 |
| 221593_s_at | RPL31 | ribosomal protein L31 | 1.757848 | 0.007055 |
| 218857_s_at | ASRGL1 | asparaginase like 1 | 1.744043 | 0.009616 |
| 210881_s_at | IGF2 | insulin-like growth factor 2 (somatomedin A) | 1.742502 | 5.58E−14 |
| 210452_x_at | CYP4F2 | cytochrome P450, family 4, subfamily F, polypeptide 2 | 1.741893 | 1.80E−06 |
| 212305_s_at | KIAA0268 /// | C219-reactive peptide /// AAAP6077 /// similar to C219-reactive | 1.734093 | 5.87E−06 |
| | UNQ6077 /// | peptide | | |
| | LOC440751 | | | |
| 219050_s_at | ZNHIT2 | zinc finger, HIT type 2 | 1.732035 | 3.14E−09 |
| 206666_at | GZMK | granzyme K (granzyme 3; tryptase II) /// granzyme K (granzyme | 1.720426 | 7.11E−06 |
| | | 3; tryptase II) | | |
| 211813_x_at | DCN | decorin | 1.719793 | 8.78E−05 |
| 204919_at | PRR4 | proline rich 4 (lacrimal) | 1.713232 | 0.000424 |
| 202270_at | GBP1 | guanylate binding protein 1, interferon-inducible, 67 kDa /// | 1.711127 | 0.006492 |
| | | guanylate binding protein 1, interferon-inducible, 67 kDa | | |
| 207114_at | LY6G6C | lymphocyte antigen 6 complex, locus G6C | 1.703258 | 3.10E−13 |
| 205987_at | CD1C | CD1C antigen, c polypeptide | 1.695706 | 2.06E−05 |
| 209325_s_at | RGS16 | regulator of G-protein signalling 16 | 1.693634 | 2.42E−07 |
| 219725_at | TREM2 | triggering receptor expressed on myeloid cells 2 | 1.693001 | 7.13E−08 |
| 205518_s_at | CMAH | cytidine monophosphate-N-acetylneuraminic acid hydroxylase | 1.684399 | 0.002381 |
| | | (CMP-N-acetylneuraminate monooxygenase) | | |
| 204122_at | TYROBP | TYRO protein tyrosine kinase binding protein | 1.677151 | 0.000344 |
| 221701_s_at | STRA6 | stimulated by retinoic acid gene 6 homolog (mouse) /// stimulated | 1.669373 | 2.01E−06 |
| | | by retinoic acid gene 6 homolog (mouse) | | |
| 201149_s_at | TIMP3 | TIMP metallopeptidase inhibitor 3 (Sorsby fundus dystrophy, | 1.657828 | 0.000357 |
| | | pseudoinflammatory) | | |
| 219666_at | MS4A6A | membrane-spanning 4-domains, subfamily A, member 6A | 1.655295 | 0.000556 |
| 211896_s_at | DCN | decorin | 1.652321 | 0.00251 |
| 213524_s_at | G0S2 | G0/G1switch 2 | 1.64485 | 0.001741 |
| 208306_x_at | HLA-DRB5 | Major histocompatibility complex, class II, DR beta 3 | 1.64335 | 4.75E−05 |
| 218380_at | NALP1 | NACHT, leucine rich repeat and PYD (pyrin domain) containing 1 | 1.638032 | 0.000218 |
| 210684_s_at | DLG4 | discs, large homolog 4 (Drosophila) | 1.629744 | 0.000385 |
| 201204_s_at | RRBP1 | Ribosome binding protein 1 homolog 180 kDa (dog) | 1.629572 | 0.005089 |
| 209869_at | ADRA2A | adrenergic, alpha-2A-, receptor /// adrenergic, alpha-2A-, | 1.624862 | 0.003948 |
| | | receptor | | |
| 210063_at | SARDH | sarcosine dehydrogenase | 1.620661 | 9.60E−05 |
| 206932_at | CH25H | cholesterol 25-hydroxylase | 1.614504 | 0.001957 |
| 202637_s_at | ICAM1 | intercellular adhesion molecule 1 (CD54), human rhinovirus | 1.613227 | 0.000977 |
| | | receptor | | |
| 204036_at | EDG2 | endothelial differentiation, lysophosphatidic acid G-protein- | 1.612794 | 0.005969 |
| | | coupled receptor, 2 | | |
| 221923_s_at | NPM1 | nucleophosmin (nucleolar phosphoprotein B23, numatrin) | 1.607706 | 6.82E−05 |
| 209083_at | CORO1A | coronin, actin binding protein, 1A | 1.605729 | 5.68E−05 |
| 64486_at | CORO1B | coronin, actin binding protein, 1B | 1.603119 | 4.13E−07 |
| 203923_s_at | CYBB | cytochrome b-245, beta polypeptide (chronic granulomatous | 1.595935 | 2.48E−07 |
| | | disease) | | |
| 205619_s_at | MEOX1 | mesenchyme homeo box 1 | 1.593023 | 0.000432 |
| 209473_at | ENTPD1 | ectonucleoside triphosphate diphosphohydrolase 1 | 1.589396 | 7.98E−05 |
| 207691_x_at | ENTPD1 | ectonucleoside triphosphate diphosphohydrolase 1 | 1.583364 | 0.002043 |
| 213566_at | RNASE6 | ribonuclease, RNase A family, k6 /// ribonuclease, RNase A | 1.582904 | 0.003242 |
| | | family, k6 | | |
| 213603_s_at | RAC2 | ras-related C3 botulinum toxin substrate 2 (rho family, small GTP | 1.576754 | 0.006208 |
| | | binding protein Rac2) | | |
| 204595_s_at | STC1 | stanniocalcin 1 | 1.575287 | 0.000307 |
| 220584_at | FLJ22184 | hypothetical protein FLJ22184 | 1.573809 | 0.000495 |
| 217511_at | KAZALD1 | Kazal-type serine peptidase inhibitor domain 1 | 1.570805 | 8.43E−06 |
| 220951_s_at | ACF | apobec-1 complementation factor | 1.563946 | 1.14E−09 |
| 201150_s_at | TIMP3 | TIMP metallopeptidase inhibitor 3 (Sorsby fundus dystrophy, | 1.560587 | 3.64E−06 |
| | | pseudoinflammatory) | | |
| 220818_s_at | TRPC4 | transient receptor potential cation channel, subfamily C, member 4 | 1.555885 | 3.57E−05 |
| 220894_x_at | PRDM12 | PR domain containing 12 | 1.554104 | 0.000536 |
| 205067_at | IL1B | interleukin 1, beta | 1.553715 | 0.001479 |
| 201426_s_at | VIM | vimentin | 1.551604 | 0.000102 |
| 215864_at | INPP4B | Inositol polyphosphate-4-phosphatase, type II, 105 kDa | 1.549052 | 1.19E−07 |
| 52255_s_at | COL5A3 | collagen, type V, alpha 3 | 1.546181 | 6.91E−26 |
| 204118_at | CD48 | CD48 antigen (B-cell membrane protein) /// CD48 antigen (B-cell | 1.543715 | 0.001868 |
| | | membrane protein) | | |
| 204655_at | CCL5 | chemokine (C-C motif) ligand 5 /// chemokine (C-C motif) ligand 5 | 1.538412 | 0.00225 |
| 210051_at | RAPGEF3 | Rap guanine nucleotide exchange factor (GEF) 3 | 1.536953 | 0.002695 |
| 208189_s_at | MYO7A | myosin VIIA | 1.531819 | 8.82E−05 |
| 220827_at | CACNA1C | Calcium channel, voltage-dependent, L type, alpha 1C subunit | 1.531756 | 2.71E−10 |
| 219407_s_at | LAMC3 | laminin, gamma 3 | 1.531194 | 3.10E−05 |
| 201422_at | IFI30 | interferon, gamma-inducible protein 30 | 1.528154 | 0.003337 |
| 202283_at | SERPINF1 | serpin peptidase inhibitor, clade F (alpha-2 antiplasmin, pigment | 1.52572 | 0.007076 |
| | | epithelium derived factor), member 1 | | |
| 221372_s_at | P2RX2 | purinergic receptor P2X, ligand-gated ion channel, 2 | 1.523015 | 3.08E−08 |
| 205859_at | LY86 | lymphocyte antigen 86 | 1.522632 | 8.40E−08 |
| 201487_at | CTSC | cathepsin C | 1.520159 | 0.00021 |
| 207704_s_at | GAS7 | growth arrest-specific 7 | 1.515128 | 3.24E−09 |
| 206852_at | EPHA7 | EPH receptor A7 | 1.51479 | 0.002947 |
| 216464_x_at | GPR44 | G protein-coupled receptor 44 | 1.514045 | 2.60E−07 |
| 206090_s_at | DISC1 | disrupted in schizophrenia 1 | 1.505648 | 0.00607 |
| 204440_at | CD83 | CD83 antigen (activated B lymphocytes, immunoglobulin | 1.500999 | 3.29E−05 |
| | | superfamily) | | |
| 206756_at | CHST7 | carbohydrate (N-acetylglucosamine 6-O) sulfotransferase 7 | 1.500986 | 1.11E−08 |
| 205582_s_at | GGTLA1 | gamma-glutamyltransferase-like activity 1 | 1.493693 | 0.004216 |
| 204852_s_at | PTPN7 | protein tyrosine phosphatase, non-receptor type 7 | 1.493524 | 2.75E−05 |
| 214219_x_at | MAP4K1 | mitogen-activated protein kinase kinase kinase kinase 1 | 1.493364 | 1.16E−09 |
| 203813_s_at | SLIT3 | slit homolog 3 (Drosophila) | 1.490279 | 4.88E−06 |
| 33197_at | MYO7A | myosin VIIA | 1.48536 | 1.67E−05 |
| 209824_s_at | ARNTL | aryl hydrocarbon receptor nuclear translocator-like | 1.484842 | 5.05E−05 |
| 210785_s_at | C1orf38 | chromosome 1 open reading frame 38 | 1.478611 | 0.000219 |
| 214781_at | — | — | 1.478349 | 5.38E−06 |
| 208966_x_at | IFI16 | interferon, gamma-inducible protein 16 | 1.471898 | 0.004326 |
| 202827_s_at | MMP14 | matrix metallopeptidase 14 (membrane-inserted) | 1.468315 | 0.006623 |
| 219993_at | SOX17 | SRY (sex determining region Y)-box 17 | 1.468222 | 9.01E−05 |
| 206883_x_at | GP9 | glycoprotein IX (platelet) | 1.462466 | 1.19E−12 |
| 206498_at | OCA2 | oculocutaneous albinism II (pink-eye dilution homolog, mouse) | 1.454155 | 3.50E−06 |
| 206978_at | CCR2 | chemokine (C-C motif) receptor 2 /// chemokine (C-C motif) | 1.453387 | 1.88E−05 |
| | | receptor 2 | | |
| 210971_s_at | ARNTL | aryl hydrocarbon receptor nuclear translocator-like | 1.452728 | 1.10E−08 |
| 221840_at | PTPRE | protein tyrosine phosphatase, receptor type, E | 1.450219 | 0.008859 |
| 201721_s_at | LAPTM5 | lysosomal associated multispanning membrane protein 5 | 1.449572 | 0.002062 |
| 221065_s_at | CHST8 | carbohydrate (N-acetylgalactosamine 4-0) sulfotransferase 8 | 1.449315 | 4.63E−06 |
| 219143_s_at | RPP25 | ribonuclease P 25 kDa subunit | 1.446895 | 9.50E−06 |
| 216842_x_at | RBMY1A1 /// | RNA binding motif protein, Y-linked, family 1, member A1 /// RNA | 1.446631 | 2.73E−13 |
| | RBMY2FP /// | binding motif protein, Y-linked, family 2, member F pseudogene | | |
| | RBMY1F /// | /// RNA binding motif protein, Y-linked, family 1, member F /// | | |
| | RBMY1B /// | RNA binding motif protein, Y-linked, family 1, member B /// RNA | | |
| | RBMY1D /// | binding motif protein, Y-linked, family 1, member D /// RNA | | |
| | RBMY1E /// | binding motif protein, Y-linked, family 1, member E /// RNA | | |
| | RBMY1J | binding motif protein, Y-linked, family 1, member J | | |
| 203508_at | TNFRSF1B | tumor necrosis factor receptor superfamily, member 1B | 1.443054 | 2.60E−09 |
| 216601_at | LOC90586 | AOC3 pseudogene | 1.438038 | 1.14E−06 |
| 211656_x_at | HLA-DQB1 | major histocompatibility complex, class II, DQ beta 1 /// major | 1.437311 | 0.004604 |
| | | histocompatibility complex, class II, DQ beta 1 | | |
| 210288_at | KLRG1 | killer cell lectin-like receptor subfamily G, member 1 | 1.436072 | 3.71E−06 |
| 203417_at | MFAP2 | microfibrillar-associated protein 2 | 1.435479 | 0.00902 |
| 215253_s_at | DSCR1 | Down syndrome critical region gene 1 | 1.432121 | 4.58E−05 |
| 203088_at | FBLN5 | fibulin 5 | 1.430714 | 0.001472 |
| 215161_at | CAMK1G | calcium/calmodulin-dependent protein kinase IG | 1.430099 | 6.76E−07 |
| 206105_at | AFF2 | AF4/FMR2 family, member 2 | 1.42814 | 1.18E−05 |
| 203761_at | SLA | Src-like-adaptor /// Src-like-adaptor | 1.42803 | 0.00108 |
| 213176_s_at | LTBP4 | latent transforming growth factor beta binding protein 4 | 1.425834 | 0.001008 |
| 218072_at | COMMD9 | COMM domain containing 9 | 1.423254 | 7.50E−07 |
| 203591_s_at | CSF3R | colony stimulating factor 3 receptor (granulocyte) /// colony | 1.422208 | 4.64E−06 |
| | | stimulating factor 3 receptor (granulocyte) | | |
| 211888_x_at | CASP10 | caspase 10, apoptosis-related cysteine peptidase | 1.420268 | 4.45E−08 |
| 204393_s_at | ACPP | acid phosphatase, prostate | 1.418571 | 0.002979 |
| 205752_s_at | GSTM5 | glutathione S-transferase M5 | 1.417412 | 0.005095 |
| 218744_s_at | PACSIN3 | protein kinase C and casein kinase substrate in neurons 3 | 1.413561 | 3.69E−07 |
| 219014_at | PLAC8 | placenta-specific 8 | 1.408531 | 0.004038 |
| 221409_at | OR2S2 | olfactory receptor, family 2, subfamily S, member 2 | 1.407145 | 3.32E−06 |
| 218047_at | OSBPL9 | oxysterol binding protein-like 9 | 1.406243 | 0.000941 |
| 205014_at | FGFBP1 | fibroblast growth factor binding protein 1 | 1.405974 | 0.000149 |
| 205915_x_at | GRIN1 | glutamate receptor, ionotropic, N-methyl D-aspartate 1 | 1.404451 | 0.001843 |
| 209993_at | ABCB1 | ATP-binding cassette, sub-family B (MDR/TAP), member 1 | 1.403813 | 0.000736 |
| 203047_at | STK10 | serine/threonine kinase 10 | 1.40223 | 4.81E−06 |
| 219916_s_at | RNF39 | ring finger protein 39 | 1.401976 | 5.02E−05 |
| 210237_at | ARTN | artemin | 1.400828 | 0.002656 |
| 213620_s_at | ICAM2 | intercellular adhesion molecule 2 | 1.40024 | 0.001242 |
| 221949_at | LOC222070 | hypothetical protein LOC222070 | 1.393796 | 3.46E−07 |
| 210321_at | GZMH | granzyme H (cathepsin G-like 2, protein h-CCPX) /// granzyme H | 1.391566 | 5.33E−08 |
| | | (cathepsin G-like 2, protein h-CCPX) | | |
| 220684_at | TBX21 | T-box 21 | 1.389553 | 9.12E−05 |
| 202972_s_at | FAM13A1 | family with sequence similarity 13, member A1 | 1.387619 | 0.001369 |
| 204923_at | CXorf9 | chromosome X open reading frame 9 | 1.385232 | 2.03E−06 |
| 209474_s_at | ENTPD1 | ectonucleoside triphosphate diphosphohydrolase 1 | 1.38397 | 0.000529 |
| 211312_s_at | WISP1 | WNT1 inducible signaling pathway protein 1 | 1.382509 | 9.48E−09 |
| 213745_at | ATRNL1 | attractin-like 1 | 1.382329 | 9.43E−10 |
| 202465_at | PCOLCE | procollagen C-endopeptidase enhancer | 1.374989 | 0.00685 |
| 217442_at | IGSF4B | Immunoglobulin superfamily, member 4B | 1.374732 | 2.13E−11 |
| 209734_at | NCKAP1L | NCK-associated protein 1-like | 1.374667 | 3.85E−06 |
| 202012_s_at | EXT2 | exostoses (multiple) 2 | 1.374388 | 4.43E−06 |
| 213744_at | ATRNL1 | attractin-like 1 | 1.373034 | 1.90E−07 |
| 207671_s_at | VMD2 | vitelliform macular dystrophy 2 (Best disease, bestrophin) | 1.372551 | 3.05E−11 |
| 219550_at | ROBO3 | roundabout, axon guidance receptor, homolog 3 (Drosophila) | 1.37161 | 0.004909 |
| 205159_at | CSF2RB | colony stimulating factor 2 receptor, beta, low-affinity | 1.370481 | 5.48E−05 |
| | | (granulocyte-macrophage) /// colony stimulating factor 2 | | |
| | | receptor, beta, low-affinity (granulocyte-macrophage) | | |
| 215283_at | LOC400642 | hypothetical gene supported by BC041875; BX648984 | 1.370296 | 6.23E−05 |
| 206296_x_at | MAP4K1 | mitogen-activated protein kinase kinase kinase kinase 1 | 1.367559 | 1.27E−06 |
| 211483_x_at | CAMK2B | calcium/calmodulin-dependent protein kinase (CaM kinase) II | 1.365512 | 4.47E−05 |
| | | beta | | |
| 207389_at | GP1BA | glycoprotein Ib (platelet), alpha polypeptide | 1.358464 | 9.64E−11 |
| 221610_s_at | STAP2 | signal-transducing adaptor protein-2 | 1.356235 | 0.001381 |
| 203423_at | RBP1 | retinol binding protein 1, cellular | 1.353443 | 1.52E−06 |
| 220331_at | CYP46A1 | cytochrome P450, family 46, subfamily A, polypeptide 1 | 1.349657 | 4.10E−05 |
| 221356_x_at | P2RX2 | purinergic receptor P2X, ligand-gated ion channel, 2 | 1.349092 | 9.10E−05 |
| 207244_x_at | CYP2A6 | cytochrome P450, family 2, subfamily A, polypeptide 6 | 1.347315 | 1.60E−05 |
| 215869_at | ABCA1 | ATP-binding cassette, sub-family A (ABC1), member 1 | 1.345443 | 1.63E−06 |
| 218870_at | ARHGAP15 | Rho GTPase activating protein 15 | 1.343762 | 0.000274 |
| 210263_at | KCNF1 | potassium voltage-gated channel, subfamily F, member 1 | 1.342529 | 3.03E−05 |
| 203562_at | FEZ1 | fasciculation and elongation protein zeta 1 (zygin I) | 1.341803 | 2.62E−06 |
| 205602_x_at | PSG7 | pregnancy specific beta-1-glycoprotein 7 | 1.338459 | 0.00018 |
| 208606_s_at | WNT4 | wingless-type MMTV integration site family, member 4 /// | 1.337541 | 0.000233 |
| | | wingless-type MMTV integration site family, member 4 | | |
| 207578_s_at | HTR4 | 5-hydroxytryptamine (serotonin) receptor 4 | 1.337137 | 1.09E−05 |
| 203391_at | FKBP2 | FK506 binding protein 2, 13 kDa | 1.336908 | 0.001163 |
| 207442_at | CSF3 | colony stimulating factor 3 (granulocyte) | 1.336667 | 8.72E−06 |
| 211898_s_at | EPHB1 | EPH receptor B1 | 1.335632 | 5.09E−08 |
| 206481_s_at | LDB2 | LIM domain binding 2 | 1.333839 | 0.003625 |
| 213854_at | SYNGR1 | synaptogyrin 1 | 1.332841 | 1.22E−08 |
| 220275_at | CUZD1 | CUB and zona pellucida-like domains 1 | 1.332314 | 5.94E−10 |
| 209335_at | DCN | decorin | 1.332115 | 0.008296 |
| 204677_at | CDH5 | cadherin 5, type 2, VE-cadherin (vascular epithelium) | 1.331911 | 0.003247 |
| 220323_at | FLJ13265 | hypothetical protein FLJ13265 | 1.328675 | 4.00E−06 |
| 216033_s_at | FYN | FYN oncogene related to SRC, FGR, YES | 1.326662 | 0.001122 |
| 217143_s_at | TRA@ /// TRD@ | T cell receptor alpha locus /// T cell receptor delta locus | 1.325576 | 2.34E−07 |
| 205575_at | C1QL1 | complement component 1, q subcomponent-like 1 | 1.320599 | 1.46E−06 |
| 206185_at | CRYBB1 | crystallin, beta B1 | 1.316754 | 4.21E−06 |
| 204232_at | FCER1G | Fc fragment of IgE, high affinity I, receptor for; gamma | 1.316049 | 0.006303 |
| | | polypeptide | | |
| 206901_at | MGC11271 | hypothetical protein MGC11271 | 1.314038 | 6.63E−08 |
| 204046_at | PLCB2 | phospholipase C, beta 2 | 1.313606 | 1.11E−10 |
| 202921_s_at | ANK2 | ankyrin 2, neuronal | 1.311235 | 4.22E−05 |
| 204174_at | ALOX5AP | arachidonate 5-lipoxygenase-activating protein | 1.30909 | 0.002048 |
| 218975_at | COL5A3 | collagen, type V, alpha 3 | 1.308812 | 3.78E−06 |
| 200862_at | DHCR24 | 24-dehydrocholesterol reductase | 1.308416 | 0.000115 |
| 206690_at | ACCN1 | amiloride-sensitive cation channel 1, neuronal (degenerin) | 1.308361 | 4.65E−07 |
| 219607_s_at | MS4A4A | membrane-spanning 4-domains, subfamily A, member 4 | 1.306072 | 0.00058 |
| 218931_at | RAB17 | RAB17, member RAS oncogene family | 1.305796 | 0.000941 |
| 203471_s_at | PLEK | pleckstrin | 1.303243 | 2.73E−05 |
| 216670_at | KLK13 | kallikrein 13 | 1.301028 | 8.63E−06 |
| 220114_s_at | STAB2 | stabilin 2 | 1.300534 | 1.10E−05 |
| 203131_at | PDGFRA | platelet-derived growth factor receptor, alpha polypeptide | 1.300041 | 0.006105 |
| 205049_s_at | CD79A | CD79A antigen (immunoglobulin-associated alpha) /// CD79A | 1.298628 | 7.07E−05 |
| | | antigen (immunoglobulin-associated alpha) | | |
| 222033_s_at | FLT1 | Fms-related tyrosine kinase 1 (vascular endothelial growth | 1.294185 | 3.73E−05 |
| | | factor/vascular permeability factor receptor) | | |
| 33579_i_at | GALR3 | galanin receptor 3 | 1.293842 | 7.63E−05 |
| 208658_at | PDIA4 | protein disulfide isomerase family A, member 4 | 1.292984 | 0.001238 |
| 211177_s_at | TXNRD2 | thioredoxin reductase 2 | 1.292756 | 0.008969 |
| 217700_at | MGC40499 | Hypothetical protein MGC40499 | 1.289032 | 0.002271 |
| 220934_s_at | MGC3196 | hypothetical protein MGC3196 | 1.28763 | 5.49E−08 |
| 219173_at | MYO15B | myosin XVB pseudogene | 1.286818 | 0.003646 |
| 203028_s_at | CYBA | cytochrome b-245, alpha polypeptide | 1.286411 | 0.001666 |
| 212611_at | DTX4 | deltex 4 homolog (Drosophila) | 1.283227 | 0.001018 |
| 204912_at | IL10RA | interleukin 10 receptor, alpha | 1.279827 | 0.002413 |
| 213095_x_at | AIF1 | allograft inflammatory factor 1 | 1.279464 | 2.76E−05 |
| 215333_x_at | GSTM1 | glutathione S-transferase M1 | 1.279061 | 0.0003 |
| 206130_s_at | ASGR2 | asialoglycoprotein receptor 2 | 1.278545 | 8.22E−05 |
| 203823_at | RGS3 | regulator of G-protein signalling 3 | 1.278094 | 0.007327 |
| 216010_x_at | FUT3 | fucosyltransferase 3 (galactoside 3(4)-L-fucosyltransferase, | 1.277343 | 3.58E−09 |
| | | Lewis blood group included) | | |
| 219594_at | NINJ2 | ninjurin 2 | 1.274351 | 0.003505 |
| 205116_at | LAMA2 | laminin, alpha 2 (merosin, congenital muscular dystrophy) | 1.272944 | 4.38E−06 |
| 209901_x_at | AIF1 | allograft inflammatory factor 1 | 1.271631 | 6.47E−05 |
| 215051_x_at | AIF1 | allograft inflammatory factor 1 | 1.27141 | 0.001009 |
| 204468_s_at | TIE1 | tyrosine kinase with immunoglobulin-like and EGF-like domains 1 | 1.268268 | 0.004301 |
| 218961_s_at | PNKP | polynucleotide kinase 3′-phosphatase | 1.266761 | 0.000323 |
| 217001_x_at | HLA-DOA | major histocompatibility complex, class II, DO alpha | 1.265885 | 2.33E−05 |
| 203547_at | CD4 | CD4 antigen (p55) /// CD4 antigen (p55) | 1.264987 | 1.15E−07 |
| 206192_at | CDSN | corneodesmosin | 1.262778 | 2.20E−06 |
| 205098_at | CCR1 | chemokine (C-C motif) receptor 1 | 1.262468 | 2.33E−05 |
| 214355_x_at | CTAGE4 /// | CTAGE family, member 4 /// similar to FLJ00261 protein /// | 1.261934 | 0.000487 |
| | LOC441296 /// | hypothetical protein LOC442780 | | |
| | LOC442780 | | | |
| 211465_x_at | FUT6 | fucosyltransferase 6 (alpha (1,3) fucosyltransferase) | 1.261367 | 0.000632 |
| 203388_at | ARRB2 | arrestin, beta 2 | 1.260163 | 3.70E−07 |
| 211439_at | SFRS7 | splicing factor, arginine/serine-rich 7, 35 kDa | 1.256575 | 8.70E−05 |
| 213661_at | DKFZP586H2123 | regeneration associated muscle protease | 1.256497 | 0.004417 |
| 204685_s_at | ATP2B2 | ATPase, Ca++ transporting, plasma membrane 2 | 1.256397 | 0.000148 |
| 208018_s_at | HCK | hemopoietic cell kinase | 1.255764 | 1.28E−05 |
| 204882_at | ARHGAP25 | Rho GTPase activating protein 25 | 1.254924 | 0.002311 |
| 211107_s_at | AURKC | aurora kinase C | 1.254824 | 0.000197 |
| 213877_x_at | TCEB2 | transcription elongation factor B (SIII), polypeptide 2 (18 kDa, | 1.25469 | 7.17E−05 |
| | | elongin B) | | |
| 219134_at | ELTD1 | EGF, latrophilin and seven transmembrane domain containing 1 | 1.254081 | 0.002919 |
| 222302_at | — | — | 1.252659 | 4.84E−05 |
| 210764_s_at | CYR61 | cysteine-rich, angiogenic inducer, 61 | 1.252464 | 0.004922 |
| 210992_x_at | FCGR2C | Fc fragment of IgG, low affinity IIc, receptor for (CD32) | 1.252432 | 0.000325 |
| 219812_at | MGC2463 | hypothetical protein MGC2463 | 1.25233 | 0.000195 |
| 218198_at | DHX32 | DEAH (Asp-Glu-Ala-His) box polypeptide 32 | 1.250753 | 0.001116 |
| 220424_at | NPHS2 | nephrosis 2, idiopathic, steroid-resistant (podocin) | 1.248843 | 0.000644 |
| 215261_at | BTBD9 | BTB (POZ) domain containing 9 | 1.246809 | 0.001911 |
| 204179_at | MB | myoglobin | 1.244098 | 0.001062 |
| 216635_at | — | MRNA; cDNA DKFZp566M0524 (from clone DKFZp566M0524) | 1.24263 | 0.006897 |
| 221655_x_at | EPS8L1 | EPS8-like 1 | 1.242194 | 0.001336 |
| 221241_s_at | BCL2L14 | BCL2-like 14 (apoptosis facilitator) /// BCL2-like 14 (apoptosis | 1.240116 | 0.008184 |
| | | facilitator) | | |
| 218180_s_at | EPS8L2 | EPS8-like 2 | 1.239582 | 0.000931 |
| 205574_x_at | BMP1 | bone morphogenetic protein 1 | 1.239223 | 0.000207 |
| 214619_at | CRHR1 | corticotropin releasing hormone receptor 1 | 1.235597 | 3.86E−05 |
| 221612_at | HT017 | HT017 protein | 1.235493 | 2.30E−05 |
| 219188_s_at | LRP16 | LRP16 protein | 1.232948 | 0.000289 |
| 209166_s_at | MAN2B1 | mannosidase, alpha, class 2B, member 1 | 1.232609 | 0.006471 |
| 204683_at | ICAM2 | intercellular adhesion molecule 2 | 1.231781 | 2.80E−05 |
| 220846_s_at | FLJ31031 | Chromosome 1 open reading frame 86 | 1.231192 | 2.59E−06 |
| 205508_at | SCN1B | sodium channel, voltage-gated, type I, beta | 1.230635 | 5.10E−05 |
| 202767_at | ACP2 | acid phosphatase 2, lysosomal | 1.230351 | 0.000279 |
| 211368_s_at | CASP1 | caspase 1, apoptosis-related cysteine peptidase (interleukin 1, | 1.22893 | 9.67E−05 |
| | | beta, convertase) | | |
| 205911_at | PTHR1 | parathyroid hormone receptor 1 | 1.227918 | 0.001313 |
| 221306_at | GPR27 | G protein-coupled receptor 27 | 1.225707 | 3.54E−07 |
| 206118_at | STAT4 | signal transducer and activator of transcription 4 | 1.225404 | 6.36E−05 |
| 206868_at | STARD8 | START domain containing 8 | 1.225035 | 0.001739 |
| 204265_s_at | GPSM3 | G-protein signalling modulator 3 (AGS3-like, C. elegans) | 1.222665 | 9.26E−05 |
| 201893_x_at | DCN | decorin | 1.219407 | 0.002361 |
| 216431_at | — | — | 1.217426 | 0.000193 |
| 220758_s_at | ROBO4 | roundabout homolog 4, magic roundabout (Drosophila) | 1.215253 | 2.55E−09 |
| 201415_at | GSS | glutathione synthetase | 1.214211 | 0.003929 |
| 219740_at | FLJ12505 | hypothetical protein FLJ12505 | 1.213626 | 0.006106 |
| 221134_at | ANGPT4 | angiopoietin 4 | 1.210957 | 2.90E−06 |
| 206702_at | TEK | TEK tyrosine kinase, endothelial (venous malformations, multiple | 1.208943 | 0.000266 |
| | | cutaneous and mucosal) | | |
| 210013_at | HPX | hemopexin | 1.207515 | 4.72E−07 |
| 218753_at | XKR8 | X Kell blood group precursor-related family, member 8 | 1.207513 | 2.39E−05 |
| 212974_at | DENND3 | DENN/MADD domain containing 3 | 1.205504 | 9.79E−07 |
| 216981_x_at | SPN | sialophorin (gpL115, leukosialin, CD43) | 1.198066 | 0.001143 |
| 209949_at | NCF2 | neutrophil cytosolic factor 2 (65 kDa, chronic granulomatous | 1.196403 | 5.62E−07 |
| | | disease, autosomal 2) | | |
| 220217_x_at | SPANXC | SPANX family, member C | 1.195915 | 3.34E−05 |
| 200916_at | TAGLN2 | transgelin 2 | 1.193798 | 0.005828 |
| 206838_at | TBX19 | T-box 19 | 1.193417 | 2.83E−05 |
| 204189_at | RARG | retinoic acid receptor, gamma | 1.190107 | 4.63E−06 |
| 212494_at | TENC1 | tensin like C1 domain containing phosphatase (tensin 2) | 1.189217 | 0.00742 |
| 207484_s_at | EHMT2 | euchromatic histone-lysine N-methyltransferase 2 | 1.18355 | 3.55E−05 |
| 209057_x_at | CDC5L | CDC5 cell division cycle 5-like (S. pombe) | 1.182148 | 0.001456 |
| 203474_at | IQGAP2 | IQ motif containing GTPase activating protein 2 | 1.181481 | 0.007674 |
| 218436_at | SIL1 | SIL1 homolog, endoplasmic reticulum chaperone (S. cerevisiae) | 1.18025 | 0.000107 |
| 216145_at | DGCR14 | DiGeorge syndrome critical region gene 14 | 1.17926 | 0.001273 |
| 221119_at | FLJ20184 | hypothetical protein FLJ20184 | 1.179235 | 3.03E−05 |
| 209723_at | SERPINB9 | serpin peptidase inhibitor, clade B (ovalbumin), member 9 | 1.178844 | 0.009258 |
| 208594_x_at | LILRA6 | leukocyte immunoglobulin-like receptor, subfamily A (with TM | 1.178449 | 0.000131 |
| | | domain), member 6 | | |
| 204550_x_at | GSTM1 | glutathione S-transferase M1 | 1.175901 | 0.009622 |
| 204116_at | IL2RG | interleukin 2 receptor, gamma (severe combined | 1.175542 | 0.00388 |
| | | immunodeficiency) | | |
| 204442_x_at | LTBP4 | latent transforming growth factor beta binding protein 4 | 1.174073 | 0.004392 |
| 221314_at | GDF9 | growth differentiation factor 9 | 1.173884 | 1.20E−07 |
| 214498_at | ASIP | agouti signaling protein, nonagouti homolog (mouse) | 1.172638 | 0.000285 |
| 33307_at | CGI-96 | CGI-96 protein | 1.172066 | 0.003255 |
| 213030_s_at | PLXNA2 | plexin A2 | 1.171819 | 0.000205 |
| 215085_x_at | DLEC1 | deleted in lung and esophageal cancer 1 | 1.171125 | 5.23E−07 |
| 214560_at | FPRL2 | formyl peptide receptor-like 2 | 1.170717 | 2.43E−06 |
| 210753_s_at | EPHB1 | EPH receptor B1 | 1.170166 | 3.28E−06 |
| 204445_s_at | ALOX5 | arachidonate 5-lipoxygenase | 1.16959 | 6.51E−06 |
| 212443_at | NBEAL2 | neurobeachin-like 2 | 1.169153 | 0.001722 |
| 38149_at | ARHGAP25 | Rho GTPase activating protein 25 | 1.168602 | 3.29E−06 |
| 206475_x_at | CSH1 | chorionic somatomammotropin hormone 1 (placental lactogen) | 1.16758 | 3.38E−06 |
| 214955_at | TMPRSS6 | transmembrane protease, serine 6 | 1.167176 | 0.001639 |
| 206713_at | NTNG1 | netrin G1 | 1.166645 | 0.00048 |
| 213851_at | MGC52022 | Similar to RIKEN cDNA 1810038N08 gene | 1.16496 | 8.79E−05 |
| 218421_at | CERK | ceramide kinase | 1.16472 | 0.000359 |
| 216637_at | ITSN1 | Intersectin 1 (SH3 domain protein) | 1.164142 | 0.000137 |
| 211101_x_at | LILRA2 | leukocyte immunoglobulin-like receptor, subfamily A (with TM | 1.163844 | 5.90E−06 |
| | | domain), member 2 | | |
| 209897_s_at | SLIT2 | slit homolog 2 (Drosophila) | 1.163723 | 0.001552 |
| 205147_x_at | NCF4 | neutrophil cytosolic factor 4, 40 kDa | 1.16372 | 5.96E−05 |
| 211397_x_at | KIR2DL2 | killer cell immunoglobulin-like receptor, two domains, long | 1.16366 | 0.000218 |
| | | cytoplasmic tail, 2 | | |
| 207669_at | KRTHB3 | keratin, hair, basic, 3 | 1.163186 | 7.51E−08 |
| 206366_x_at | XCL2 | chemokine (C motif) ligand 2 | 1.162929 | 0.004354 |
| 206819_at | DKFZP434P211 | POM121-like protein | 1.162553 | 5.94E−05 |
| 215042_at | BMP6 | bone morphogenetic protein 6 | 1.161377 | 0.000244 |
| 209588_at | EPHB2 | EPH receptor B2 | 1.160957 | 1.33E−05 |
| 210629_x_at | LST1 | leukocyte specific transcript 1 | 1.158822 | 7.95E−06 |
| 210571_s_at | CMAH | cytidine monophosphate-N-acetylneuraminic acid hydroxylase | 1.158728 | 0.000676 |
| | | (CMP-N-acetylneuraminate monooxygenase) | | |
| 219559_at | C20orf59 | chromosome 20 open reading frame 59 | 1.158237 | 0.000201 |
| 217275_at | TSSK2 | testis-specific serine kinase 2 | 1.15798 | 8.86E−05 |
| 211581_x_at | LST1 | leukocyte specific transcript 1 | 1.157886 | 1.22E−07 |
| 201011_at | RPN1 | ribophorin I | 1.157681 | 0.001796 |
| 206227_at | CILP | cartilage intermediate layer protein, nucleotide | 1.155692 | 7.85E−05 |
| | | pyrophosphohydrolase | | |
| 205186_at | DNALI1 | dynein, axonemal, light intermediate polypeptide 1 | 1.154983 | 0.003285 |
| 205984_at | CRHBP | corticotropin releasing hormone binding protein | 1.15356 | 0.000122 |
| 209961_s_at | HGF | hepatocyte growth factor (hepapoietin A; scatter factor) | 1.152891 | 0.000229 |
| 213897_s_at | MRPL23 | mitochondrial ribosomal protein L23 | 1.15272 | 2.04E−06 |
| 90265_at | CENTA1 | centaurin, alpha 1 | 1.15202 | 1.23E−05 |
| 214181_x_at | LST1 | leukocyte specific transcript 1 | 1.149659 | 0.001679 |
| 218843_at | FNDC4 | fibronectin type III domain containing 4 | 1.148459 | 0.000618 |
| 214339_s_at | MAP4K1 | mitogen-activated protein kinase kinase kinase kinase 1 | 1.146132 | 0.000207 |
| 211873_s_at | PCDHGA9 | protocadherin gamma subfamily A, 9 | 1.145308 | 1.52E−05 |
| 219593_at | SLC15A3 | solute carrier family 15, member 3 | 1.144956 | 0.000108 |
| 209169_at | GPM6B | glycoprotein M6B | 1.144321 | 3.01E−06 |
| 204319_s_at | RGS10 | regulator of G-protein signalling 10 | 1.144314 | 0.001006 |
| 213112_s_at | SQSTM1 | sequestosome 1 | 1.143514 | 0.00015 |
| 209365_s_at | ECM1 | extracellular matrix protein 1 | 1.142208 | 0.000612 |
| 206804_at | CD3G | CD3G antigen, gamma polypeptide (TiT3 complex) | 1.141646 | 7.59E−08 |
| 204800_s_at | FLJ13639 | hypothetical protein FLJ13639 | 1.14114 | 0.006305 |
| 221297_at | GPRC5D | G protein-coupled receptor, family C, group 5, member D | 1.140954 | 1.86E−05 |
| 213832_at | — | Clone 24405 mRNA sequence | 1.140383 | 0.006423 |
| 219113_x_at | DHRS10 | dehydrogenase/reductase (SDR family) member 10 | 1.139395 | 0.001779 |
| 213895_at | EMP1 | epithelial membrane protein 1 | 1.139386 | 0.004267 |
| 206402_s_at | NPFF | neuropeptide FF-amide peptide precursor | 1.13774 | 0.00623 |
| 219310_at | C20orf39 | chromosome 20 open reading frame 39 | 1.137368 | 0.00271 |
| 215550_at | SRGAP3 | SLIT-ROBO Rho GTPase activating protein 3 | 1.136009 | 1.30E−08 |
| 202005_at | ST14 | suppression of tumorigenicity 14 (colon carcinoma, matriptase, | 1.1355 | 0.000832 |
| | | epithin) | | |
| 211557_x_at | SLCO2B1 | solute carrier organic anion transporter family, member 2B1 | 1.13542 | 0.009672 |
| 208474_at | CLDN6 | claudin 6 | 1.135153 | 0.000232 |
| 202947_s_at | GYPC | glycophorin C (Gerbich blood group) | 1.134178 | 0.000687 |
| 207334_s_at | TGFBR2 | transforming growth factor, beta receptor II (70/80 kDa) | 1.132974 | 0.00068 |
| 221640_s_at | LRDD | leucine-rich repeats and death domain containing | 1.132909 | 0.000615 |
| 214234_s_at | CYP3A5 | cytochrome P450, family 3, subfamily A, polypeptide 5 | 1.130604 | 0.006804 |
| 217652_at | KIAA0892 | KIAA0892 | 1.129858 | 3.28E−06 |
| 205677_s_at | DLEU1 | deleted in lymphocytic leukemia, 1 | 1.129101 | 9.99E−05 |
| 206097_at | SLC22A18AS | solute carrier family 22 (organic cation transporter), member 18 | 1.127572 | 0.001618 |
| | | antisense | | |
| 203561_at | FCGR2A | Fc fragment of IgG, low affinity IIa, receptor (CD32) | 1.12705 | 0.005208 |
| 217044_s_at | PLEKHG3 | pleckstrin homology domain containing, family G (with RhoGef | 1.1264 | 0.000122 |
| | | domain) member 3 | | |
| 220747_at | HSPC072 | HSPC072 protein | 1.126193 | 3.21E−05 |
| 221578_at | RASSF4 | Ras association (RalGDS/AF-6) domain family 4 | 1.125902 | 2.37E−06 |
| 204574_s_at | MMP19 | matrix metallopeptidase 19 | 1.125668 | 0.009642 |
| 217312_s_at | COL7A1 | collagen, type VII, alpha 1 (epidermolysis bullosa, dystrophic, | 1.125297 | 0.006292 |
| | | dominant and recessive) | | |
| 209436_at | SPON1 | spondin 1, extracellular matrix protein | 1.123601 | 0.000215 |
| 200696_s_at | GSN | gelsolin (amyloidosis, Finnish type) | 1.123572 | 0.003018 |
| 211738_x_at | ELA3A | elastase 3A, pancreatic /// elastase 3A, pancreatic | 1.123323 | 0.000461 |
| 220805_at | HRH2 | histamine receptor H2 | 1.123186 | 9.48E−07 |
| 219103_at | DDEFL1 | development and differentiation enhancing factor-like 1 | 1.120327 | 0.00046 |
| 215411_s_at | TRAF3IP2 | TRAF3 interacting protein 2 | 1.120294 | 5.14E−05 |
| 201148_s_at | TIMP3 | TIMP metallopeptidase inhibitor 3 (Sorsby fundus dystrophy, | 1.120049 | 0.000928 |
| | | pseudoinflammatory) | | |
| 213695_at | PON3 | paraoxonase 3 | 1.119743 | 5.72E−05 |
| 218965_s_at | RBM21 | RNA binding motif protein 21 | 1.11951 | 0.00334 |
| 204674_at | LRMP | lymphoid-restricted membrane protein | 1.118861 | 0.000553 |
| 218245_at | LRRC54 | leucine rich repeat containing 54 | 1.11885 | 0.000789 |
| 207376_at | VENTX | VENT homeobox homolog (Xenopus laevis) | 1.117574 | 8.52E−06 |
| 207960_at | — | — | 1.116504 | 0.000935 |
| 215610_at | — | Transcribed locus | 1.112932 | 1.05E−08 |
| 204358_s_at | FLRT2 | fibronectin leucine rich transmembrane protein 2 | 1.112246 | 0.000143 |
| 207784_at | ARSD | arylsulfatase D | 1.11146 | 2.37E−05 |
| 203409_at | DDB2 /// LHX3 | damage-specific DNA binding protein 2, 48 kDa /// LIM homeobox 3 | 1.11139 | 0.005919 |
| 219707_at | CPNE7 | copine VII | 1.110303 | 0.002104 |
| 215906_at | — | — | 1.109794 | 0.000269 |
| 212613_at | BTN3A2 | butyrophilin, subfamily 3, member A2 | 1.108914 | 0.004853 |
| 209324_s_at | RGS16 | regulator of G-protein signalling 16 | 1.108761 | 6.77E−05 |
| 215971_at | — | CDNA FLJ12058 fis, clone HEMBB1002092 | 1.107792 | 7.39E−06 |
| 205129_at | NPM3 | nucleophosmin/nucleoplasmin, 3 | 1.107268 | 0.000756 |
| 213223_at | RPL28 | ribosomal protein L28 | 1.107263 | 0.001166 |
| 209970_x_at | CASP1 | caspase 1, apoptosis-related cysteine peptidase (interleukin 1, | 1.107025 | 0.000298 |
| | | beta, convertase) | | |
| 208048_at | TACR1 | tachykinin receptor 1 | 1.107001 | 1.38E−08 |
| 208468_at | SOX21 | SRY (sex determining region Y)-box 21 | 1.106596 | 2.15E−05 |
| 215010_s_at | BRSK2 | BR serine/threonine kinase 2 | 1.105591 | 3.18E−07 |
| 217509_x_at | GRIK5 | glutamate receptor, ionotropic, kainate 5 | 1.105381 | 1.79E−05 |
| 215701_at | — | — | 1.102759 | 8.62E−05 |
| 204220_at | GMFG | glia maturation factor, gamma | 1.102296 | 0.001946 |
| 215082_at | ELOVL5 | ELOVL family member 5, elongation of long chain fatty acids | 1.101982 | 9.24E−05 |
| | | (FEN1/Elo2, SUR4/Elo3-like, yeast) | | |
| 202818_s_at | TCEB3 | transcription elongation factor B (SIII), polypeptide 3 (110 kDa, | 1.101175 | 0.000278 |
| | | elongin A) | | |
| 214357_at | C1orf105 | chromosome 1 open reading frame 105 | 1.101112 | 5.08E−05 |
| 217285_at | DGCR13 | DiGeorge syndrome critical region gene 13 | 1.100987 | 0.001335 |
| 207767_s_at | EGR4 | early growth response 4 | 1.099535 | 0.000511 |
| 201206_s_at | RRBP1 | ribosome binding protein 1 homolog 180 kDa (dog) | 1.099139 | 0.006128 |
| 215515_at | KIRREL | Kin of IRRE like (Drosophila) | 1.098938 | 0.000903 |
| 214195_at | TPP1 | tripeptidyl peptidase I | 1.098226 | 7.27E−07 |
| 217391_x_at | — | — | 1.098124 | 0.000774 |
| 210958_s_at | MAST4 | microtubule associated serine/threonine kinase family member 4 | 1.097492 | 0.008785 |
| 219821_s_at | GFOD1 | glucose-fructose oxidoreductase domain containing 1 | 1.095218 | 3.14E−07 |
| 210038_at | PRKCQ | protein kinase C, theta | 1.094495 | 0.000198 |
| 205617_at | PRRG2 | proline rich Gla (G-carboxyglutamic acid) 2 | 1.094276 | 1.11E−06 |
| 210795_s_at | MEG3 | Maternally expressed 3 | 1.093571 | 3.76E−07 |
| 201374_x_at | PPP2CB | protein phosphatase 2 (formerly 2A), catalytic subunit, beta | 1.091421 | 0.001955 |
| | | isoform | | |
| 210414_at | FLRT1 | fibronectin leucine rich transmembrane protein 1 | 1.090188 | 0.001513 |
| 216919_at | TP53I11 | tumor protein p53 inducible protein 11 | 1.090037 | 0.000901 |
| 221565_s_at | FAM26B | family with sequence similarity 26, member B | 1.08976 | 0.000534 |
| 216535_at | IGSF4B | immunoglobulin superfamily, member 4B | 1.08963 | 0.00034 |
| 220447_at | HRH3 | histamine receptor H3 | 1.089199 | 0.000484 |
| 222013_x_at | APP | amyloid beta (A4) precursor protein (peptidase nexin-II, | 1.087231 | 0.000364 |
| | | Alzheimer disease) | | |
| 210924_at | OLFM1 | olfactomedin 1 | 1.08674 | 0.000904 |
| 214050_at | — | — | 1.08424 | 0.000395 |
| 216174_at | HCRP1 | hepatocellular carcinoma-related HCRP1 | 1.083556 | 0.000134 |
| 215989_at | CBX2 | chromobox homolog 2 (Pc class homolog, Drosophila) | 1.082325 | 0.000209 |
| 220142_at | HAPLN2 | hyaluronan and proteoglycan link protein 2 | 1.082306 | 0.001665 |
| 216318_at | — | — | 1.081527 | 8.88E−06 |
| 207552_at | ATP5G2 | ATP synthase, H+ transporting, mitochondrial F0 complex, | 1.081272 | 0.00162 |
| | | subunit c (subunit 9), isoform 2 | | |
| 204770_at | TAP2 | transporter 2, ATP-binding cassette, sub-family B (MDR/TAP) | 1.080895 | 0.000451 |
| 221310_at | FGF14 | fibroblast growth factor 14 | 1.078813 | 5.24E−06 |
| 216730_at | — | CDNA: FLJ20908 fis, clone ADSE00417 | 1.078621 | 0.000573 |
| 209946_at | VEGFC | vascular endothelial growth factor C | 1.078162 | 0.003348 |
| 219716_at | APOL6 | apolipoprotein L, 6 | 1.077216 | 0.002077 |
| 219746_at | DPF3 | D4, zinc and double PHD fingers, family 3 | 1.077036 | 2.25E−05 |
| 206586_at | CNR2 | cannabinoid receptor 2 (macrophage) | 1.076953 | 0.000126 |
| 211469_s_at | CXCR6 | chemokine (C—X—C motif) receptor 6 | 1.07681 | 3.84E−05 |
| 202803_s_at | ITGB2 | integrin, beta 2 (antigen CD18 (p95), lymphocyte function- | 1.076421 | 0.009872 |
| | | associated antigen 1; macrophage antigen 1 (mac-1) beta | | |
| | | subunit) | | |
| 211125_x_at | GRIN1 | glutamate receptor, ionotropic, N-methyl D-aspartate 1 | 1.074783 | 3.85E−05 |
| 208198_x_at | KIR2DS1 | killer cell immunoglobulin-like receptor, two domains, short | 1.073912 | 0.003583 |
| | | cytoplasmic tail, 1 | | |
| 204395_s_at | GRK5 | G protein-coupled receptor kinase 5 | 1.073395 | 0.001142 |
| 219569_s_at | TMEM22 | transmembrane protein 22 | 1.073144 | 8.29E−05 |
| 221347_at | CHRM5 | cholinergic receptor, muscarinic 5 | 1.072709 | 0.003841 |
| 212032_s_at | PTOV1 | prostate tumor overexpressed gene 1 | 1.070938 | 0.003341 |
| 215689_s_at | SHBG | sex hormone-binding globulin | 1.070663 | 3.59E−05 |
| 204006_s_at | FCGR3A /// | Fc fragment of IgG, low affinity IIIa, receptor (CD16a) /// Fc | 1.069524 | 0.006449 |
| | FCGR3B | fragment of IgG, low affinity IIIb, receptor (CD16b) | | |
| 220279_at | TRIM17 | tripartite motif-containing 17 | 1.068909 | 0.002536 |
| 219152_at | PODXL2 | podocalyxin-like 2 | 1.068572 | 0.002093 |
| 216204_at | ARVCF | Armadillo repeat gene deletes in velocardiofacial syndrome | 1.067051 | 7.40E−05 |
| 208551_at | HIST1H4G | histone 1, H4g | 1.066869 | 3.87E−05 |
| 216471_x_at | SSX2 | synovial sarcoma, X breakpoint 2 | 1.064977 | 2.02E−07 |
| 216832_at | RUNX1T1 | runt-related transcription factor 1; translocated to, 1 (cyclin D- | 1.06483 | 0.000944 |
| | | related) | | |
| 222261_at | KIAA1609 | KIAA1609 protein | 1.064259 | 0.000799 |
| 213381_at | C10orf72 | Chromosome 10 open reading frame 72 | 1.064227 | 0.004801 |
| 211582_x_at | LST1 | leukocyte specific transcript 1 | 1.063432 | 0.000141 |
| 213676_at | — | Similar to expressed sequence AW125688 | 1.063194 | 0.000577 |
| 209975_at | CYP2E1 | cytochrome P450, family 2, subfamily E, polypeptide 1 | 1.05977 | 0.001648 |
| 205468_s_at | IRF5 | interferon regulatory factor 5 | 1.059766 | 2.42E−05 |
| 204844_at | ENPEP | glutamyl aminopeptidase (aminopeptidase A) | 1.058753 | 8.44E−08 |
| 211121_s_at | DOK1 | docking protein 1, 62 kDa (downstream of tyrosine kinase 1) | 1.05813 | 5.73E−05 |
| 217033_x_at | NTRK3 | neurotrophic tyrosine kinase, receptor, type 3 | 1.057614 | 0.00093 |
| 209199_s_at | MEF2C | MADS box transcription enhancer factor 2, polypeptide C | 1.057181 | 0.002233 |
| | | (myocyte enhancer factor 2C) | | |
| 214543_x_at | QKI | quaking homolog, KH domain RNA binding (mouse) | 1.057106 | 0.000111 |
| 211472_at | PLXNB2 | Plexin B2 | 1.056568 | 7.40E−05 |
| 211254_x_at | RHAG | Rhesus blood group-associated glycoprotein | 1.055625 | 0.000136 |
| 217716_s_at | SEC61A1 | Sec61 alpha 1 subunit (S. cerevisiae) | 1.055173 | 0.000467 |
| 204880_at | MGMT | O-6-methylguanine-DNA methyltransferase | 1.054187 | 0.0011 |
| 207473_at | MLN | motilin | 1.053301 | 0.000847 |
| 202828_s_at | MMP14 | matrix metallopeptidase 14 (membrane-inserted) | 1.053203 | 3.43E−06 |
| 201358_s_at | COPB | coatomer protein complex, subunit beta | 1.052447 | 0.001585 |
| 214574_x_at | LST1 | leukocyte specific transcript 1 | 1.051954 | 2.67E−07 |
| 205627_at | CDA | cytidine deaminase | 1.051772 | 1.81E−05 |
| 219170_at | FSD1 | fibronectin type III and SPRY domain containing 1 | 1.05101 | 4.82E−05 |
| 205592_at | SLC4A1 | Solute carrier family 4, anion exchanger, member 1 (erythrocyte | 1.050982 | 0.000456 |
| | | membrane protein band 3, Diego blood group) | | |
| 221982_x_at | DXS9879E | DNA segment on chromosome X (unique) 9879 expressed | 1.050854 | 2.03E−05 |
| | | sequence | | |
| 211507_s_at | MTMR3 | myotubularin related protein 3 | 1.050169 | 0.001274 |
| 206612_at | CACNG1 | calcium channel, voltage-dependent, gamma subunit 1 | 1.049659 | 9.41E−06 |
| 211809_x_at | COL13A1 | collagen, type XIII, alpha 1 | 1.046958 | 0.00606 |
| 220748_s_at | ZNF580 | zinc finger protein 580 | 1.046107 | 0.007542 |
| 207677_s_at | NCF4 | neutrophil cytosolic factor 4, 40 kDa /// neutrophil cytosolic factor | 1.044207 | 2.78E−06 |
| | | 4, 40 kDa | | |
| 202206_at | ARL7 | ADP-ribosylation factor-like 7 | 1.044112 | 0.007809 |
| 217018_at | — | — | 1.04192 | 0.000153 |
| 214398_s_at | IKBKE | inhibitor of kappa light polypeptide gene enhancer in B-cells, | 1.038623 | 2.70E−05 |
| | | kinase epsilon | | |
| 219752_at | RASAL1 | RAS protein activator like 1 (GAP1 like) | 1.03815 | 0.001556 |
| 216840_s_at | LAMA2 | laminin, alpha 2 (merosin, congenital muscular dystrophy) | 1.037806 | 0.003018 |
| 203982_s_at | ABCD4 | ATP-binding cassette, sub-family D (ALD), member 4 | 1.037405 | 5.80E−06 |
| 221010_s_at | SIRT5 | sirtuin (silent mating type information regulation 2 homolog) 5 | 1.035556 | 2.47E−06 |
| | | (S. cerevisiae) /// sirtuin (silent mating type information regulation 2 | | |
| | | homolog) 5 (S. cerevisiae) | | |
| 213592_at | AGTRL1 | angiotensin II receptor-like 1 | 1.034585 | 8.33E−05 |
| 219652_s_at | CXorf36 | chromosome X open reading frame 36 | 1.033314 | 2.06E−06 |
| 212283_at | AGRN | agrin | 1.032403 | 2.05E−05 |
| 216700_at | TRIO | Triple functional domain (PTPRF interacting) | 1.030811 | 0.000696 |
| 213706_at | GPD1 | glycerol-3-phosphate dehydrogenase 1 (soluble) | 1.030612 | 5.58E−07 |
| 204834_at | FGL2 | fibrinogen-like 2 | 1.030404 | 0.006944 |
| 222210_at | KIAA0195 | KIAA0195 gene product | 1.029435 | 2.85E−06 |
| 217357_at | — | — | 1.027727 | 0.000145 |
| 219365_s_at | CAMKV | CaM kinase-like vesicle-associated | 1.027098 | 0.000579 |
| 214487_s_at | RAP2A /// RAP2B | RAP2A, member of RAS oncogene family /// RAP2B, member of | 1.026884 | 0.000181 |
| | | RAS oncogene family | | |
| 210880_s_at | EFS | embryonal Fyn-associated substrate | 1.024848 | 0.007538 |
| 221001_at | X102 | X102 gene /// X102 gene | 1.024812 | 4.99E−07 |
| 221237_s_at | OSBP2 | oxysterol binding protein 2 /// oxysterol binding protein 2 | 1.024408 | 0.001013 |
| 206213_at | WNT10B | wingless-type MMTV integration site family, member 10B | 1.024375 | 0.000127 |
| 214549_x_at | SPRR1A | small proline-rich protein 1A | 1.024205 | 0.002567 |
| 221005_s_at | PTDSS2 | phosphatidylserine synthase 2 /// phosphatidylserine synthase 2 | 1.023927 | 0.000239 |
| 215292_s_at | MKL1 | megakaryoblastic leukemia (translocation) 1 | 1.023718 | 6.17E−05 |
| 207648_at | DRP2 | dystrophin related protein 2 | 1.023445 | 0.000731 |
| 214460_at | LSAMP | limbic system-associated membrane protein | 1.021305 | 0.009662 |
| 208320_at | CABP1 | calcium binding protein 1 (calbrain) | 1.020849 | 0.000154 |
| 201107_s_at | THBS1 | thrombospondin 1 | 1.020722 | 0.006437 |
| 213309_at | PLCL2 | phospholipase C-like 2 | 1.020016 | 7.49E−07 |
| 222029_x_at | HKE2 | HLA class II region expressed gene KE2 | 1.019963 | 0.008569 |
| 205614_x_at | MST1 | macrophage stimulating 1 (hepatocyte growth factor-like) | 1.019198 | 0.000311 |
| 207835_at | FBLN1 | fibulin 1 | 1.018196 | 0.00032 |
| 208110_x_at | MED25 | mediator of RNA polymerase II transcription, subunit 25 homolog | 1.017807 | 2.44E−06 |
| | | (yeast) /// mediator of RNA polymerase II transcription, subunit | | |
| | | 25 homolog (yeast) | | |
| 210399_x_at | FUT6 | fucosyltransferase 6 (alpha (1,3) fucosyltransferase) | 1.017012 | 0.000529 |
| 221391_at | TAS2R14 | taste receptor, type 2, member 14 | 1.016935 | 1.05E−06 |
| 89977_at | FLJ20581 | hypothetical protein FLJ20581 | 1.01608 | 0.000219 |
| 210310_s_at | FGF5 | fibroblast growth factor 5 | 1.014682 | 9.00E−05 |
| 216343_at | PCDHGA3 | protocadherin gamma subfamily A, 3 | 1.01452 | 0.000553 |
| 214750_at | PLAC4 | placenta-specific 4 | 1.014185 | 0.000205 |
| 213681_at | CYHR1 | cysteine/histidine-rich 1 | 1.014052 | 0.004029 |
| 216355_at | PCDHB17 | protocadherin beta 17 pseudogene | 1.013899 | 1.86E−05 |
| 215833_s_at | SPPL2B | signal peptide peptidase-like 2B | 1.013712 | 0.001556 |
| 215461_at | ZNRF4 | zinc and ring finger 4 | 1.013446 | 0.000183 |
| 207421_at | CA5A | carbonic anhydrase VA, mitochondrial | 1.013271 | 5.34E−05 |
| 204153_s_at | MFNG | manic fringe homolog (Drosophila) | 1.013229 | 4.86E−05 |
| 215540_at | TRA@ | T cell receptor alpha locus | 1.013047 | 3.91E−06 |
| 214227_at | GNG7 | Guanine nucleotide binding protein (G protein), gamma 7 | 1.012821 | 1.33E−07 |
| 211230_s_at | PIK3CD | phosphoinositide-3-kinase, catalytic, delta polypeptide | 1.012684 | 0.000832 |
| 221300_at | C15orf2 | chromosome 15 open reading frame 2 | 1.011462 | 6.27E−05 |
| 215633_x_at | LST1 | leukocyte specific transcript 1 | 1.011338 | 0.000293 |
| 206863_x_at | — | — | 1.010909 | 0.000248 |
| 215715_at | SLC6A2 | solute carrier family 6 (neurotransmitter transporter, | 1.010879 | 1.45E−05 |
| | | noradrenalin), member 2 | | |
| 220061_at | FLJ20581 | hypothetical protein FLJ20581 | 1.010812 | 9.24E−05 |
| 200743_s_at | TPP1 | tripeptidyl peptidase I | 1.010374 | 0.007319 |
| 209977_at | PLG | plasminogen | 1.009697 | 3.08E−08 |
| 206967_at | CCNT1 | cyclin T1 | 1.009678 | 0.002755 |
| 336_at | TBXA2R | thromboxane A2 receptor | 1.009425 | 0.005581 |
| 213673_x_at | SCIRP10 | Neuron derived neurotrophic factor | 1.009335 | 0.000132 |
| 214646_at | HIST1H3J | Histone 1, H3j | 1.009183 | 0.001027 |
| 208250_s_at | DMBT1 | deleted in malignant brain tumors 1 | 1.00721 | 0.000584 |
| 219815_at | GAL3ST4 | galactose-3-O-sulfotransferase 4 | 1.006456 | 8.73E−05 |
| 206811_at | ADCY8 | adenylate cyclase 8 (brain) | 1.006309 | 7.18E−05 |
| 215798_at | ALDH1L1 | aldehyde dehydrogenase 1 family, member L1 | 1.006185 | 0.004725 |
| 208014_x_at | AD7C-NTP | neuronal thread protein AD7c-NTP | 1.005731 | 0.004158 |
| 202665_s_at | WASPIP | Wiskott-Aldrich syndrome protein interacting protein | 1.005529 | 3.84E−06 |
| 220538_at | ADM2 | adrenomedullin 2 | 1.00539 | 0.000509 |
| 205688_at | TFAP4 | transcription factor AP-4 (activating enhancer binding protein 4) | 1.004625 | 0.000174 |
| 209873_s_at | PKP3 | plakophilin 3 | 1.003699 | 0.003234 |
| 211541_s_at | DYRK1A | dual-specificity tyrosine-(Y)-phosphorylation regulated kinase 1A | 1.003632 | 0.001865 |
| 220027_s_at | RASIP1 | Ras interacting protein 1 | 1.003009 | 0.005147 |
| 204811_s_at | CACNA2D2 | calcium channel, voltage-dependent, alpha 2/delta subunit 2 | 1.002381 | 0.000191 |
| 221844_x_at | — | CDNA clone IMAGE: 6208446 | 1.001411 | 0.002373 |
| 207933_at | ZP2 | zona pellucida glycoprotein 2 (sperm receptor) | 1.00048 | 3.25E−05 |
| 206913_at | BAAT | bile acid Coenzyme A: amino acid N-acyltransferase (glycine N- | 0.99993 | 3.36E−05 |
| | | choloyltransferase) | | |
| 208257_x_at | PSG1 | pregnancy specific beta-1-glycoprotein 1 | 0.999826 | 0.008624 |
| 206560_s_at | MIA | melanoma inhibitory activity | 0.999537 | 0.005338 |
| 217423_at | TTLL2 | Tubulin tyrosine ligase-like family, member 2 | 0.999515 | 0.00178 |
| 216143_at | CLN6 | Ceroid-lipofuscinosis, neuronal 6, late infantile, variant | 0.999408 | 1.13E−05 |
| 211100_x_at | LILRA2 | leukocyte immunoglobulin-like receptor, subfamily A (with TM | 0.999034 | 5.40E−07 |
| | | domain), member 2 | | |
| 205032_at | ITGA2 | integrin, alpha 2 (CD49B, alpha 2 subunit of VLA-2 receptor) | 0.998939 | 0.002165 |
| 217108_at | — | — | 0.998791 | 0.002145 |
| 206327_s_at | CDH15 | cadherin 15, M-cadherin (myotubule) | 0.998169 | 0.001107 |
| 215502_at | — | IMAGE cDNA clone 26881 | 0.997939 | 0.002203 |
| 35150_at | CD40 | CD40 antigen (TNF receptor superfamily member 5) | 0.997766 | 0.000269 |
| 210619_s_at | HYAL1 | hyaluronoglucosaminidase 1 | 0.996516 | 8.49E−06 |
| 209320_at | ADCY3 | adenylate cyclase 3 | 0.996384 | 0.000106 |
| 207377_at | PPP1R2P9 | protein phosphatase 1, regulatory (inhibitor) subunit 2 | 0.996091 | 1.12E−06 |
| | | pseudogene 9 | | |
| 216375_s_at | ETV5 | ets variant gene 5 (ets-related molecule) | 0.995764 | 5.57E−05 |
| 209906_at | C3AR1 | complement component 3a receptor 1 | 0.994516 | 0.005283 |
| 209334_s_at | PSMD9 | proteasome (prosome, macropain) 26S subunit, non-ATPase, 9 | 0.993686 | 0.000779 |
| 38671_at | PLXND1 | plexin D1 | 0.993379 | 0.007427 |
| 209197_at | SYT11 | synaptotagmin XI | 0.993298 | 0.000702 |
| 217207_s_at | BTNL3 | butyrophilin-like 3 | 0.992646 | 0.003392 |
| 203922_s_at | CYBB | cytochrome b-245, beta polypeptide (chronic granulomatous | 0.992087 | 0.000227 |
| | | disease) | | |
| 216767_at | PARVA | Parvin, alpha | 0.9917 | 0.000276 |
| 215951_at | KIAA1055 | KIAA1055 protein | 0.991587 | 6.06E−07 |
| 214866_at | PLAUR | plasminogen activator, urokinase receptor | 0.991117 | 0.000182 |
| 220151_at | FLJ10490 | hypothetical protein FLJ10490 | 0.990848 | 3.63E−07 |
| 220125_at | DNAI1 | dynein, axonemal, intermediate polypeptide 1 | 0.990424 | 0.000197 |
| 221627_at | TRIM10 | tripartite motif-containing 10 | 0.989742 | 0.000151 |
| 220798_x_at | PRG2 | plasticity-related gene 2 | 0.989689 | 0.000961 |
| 207878_at | KRT2B | cytokeratin 2 | 0.989357 | 0.000104 |
| 218644_at | PLEK2 | pleckstrin 2 | 0.988125 | 0.000537 |
| 216034_at | SUHW1 | suppressor of hairy wing homolog 1 (Drosophila) | 0.988102 | 0.000254 |
| 221792_at | RAB6B | RAB6B, member RAS oncogene family | 0.987376 | 0.000358 |
| 207610_s_at | EMR2 | egf-like module containing, mucin-like, hormone receptor-like 2 | 0.986348 | 1.60E−06 |
| 214617_at | PRF1 | perforin 1 (pore forming protein) /// perforin 1 (pore forming | 0.986237 | 0.000203 |
| | | protein) | | |
| 205786_s_at | ITGAM | integrin, alpha M (complement component receptor 3, alpha; also | 0.98524 | 0.000228 |
| | | known as CD11b (p170), macrophage antigen alpha | | |
| | | polypeptide) /// integrin, alpha M (complement component | | |
| | | receptor 3, alpha; also known as CD11b (p170), macrophage | | |
| | | antigen alpha polypeptide) | | |
| 211203_s_at | CNTN1 | contactin 1 | 0.985236 | 0.002938 |
| 220006_at | FLJ12057 | hypothetical protein FLJ12057 | 0.984517 | 0.003694 |
| 203729_at | EMP3 | epithelial membrane protein 3 | 0.983296 | 0.004107 |
| 202273_at | PDGFRB | platelet-derived growth factor receptor, beta polypeptide | 0.981187 | 0.004208 |
| 204495_s_at | DKFZP434H132 | DKFZP434H132 protein | 0.98108 | 0.002842 |
| 214194_at | KIAA1008 | KIAA1008 | 0.981061 | 0.000111 |
| 216320_x_at | MST1 | macrophage stimulating 1 (hepatocyte growth factor-like) | 0.980436 | 0.000234 |
| 205082_s_at | AOX1 | aldehyde oxidase 1 | 0.980009 | 0.006711 |
| 206362_x_at | MAP3K10 | mitogen-activated protein kinase kinase kinase 10 | 0.979762 | 6.68E−05 |
| 208391_s_at | GLP1R | glucagon-like peptide 1 receptor | 0.979549 | 0.001333 |
| 203666_at | CXCL12 | chemokine (C—X—C motif) ligand 12 (stromal cell-derived factor 1) | 0.979442 | 0.002696 |
| 219403_s_at | HPSE | heparanase | 0.978728 | 5.65E−09 |
| 211806_s_at | KCNJ15 | potassium inwardly-rectifying channel, subfamily J, member 15 | 0.977504 | 0.002924 |
| 220737_at | RPS6KA6 | ribosomal protein S6 kinase, 90 kDa, polypeptide 6 | 0.976875 | 6.18E−06 |
| 220727_at | KCNK10 | potassium channel, subfamily K, member 10 | 0.975527 | 0.000212 |
| 220678_at | FLJ20712 | hypothetical protein FLJ20712 | 0.974765 | 3.79E−06 |
| 213323_s_at | ZC3H7B | zinc finger CCCH-type containing 7B | 0.97462 | 2.91E−05 |
| 206673_at | GPR | putative G protein coupled receptor | 0.973485 | 0.000642 |
| 211718_at | MGC2889 | hypothetical protein MGC2889 /// hypothetical protein MGC2889 | 0.973295 | 3.41E−05 |
| 207968_s_at | MEF2C | MADS box transcription enhancer factor 2, polypeptide C | 0.972413 | 0.000414 |
| | | (myocyte enhancer factor 2C) | | |
| 213685_at | TCEB3 | Transcription elongation factor B (SIII), polypeptide 3 (110 kDa, | 0.972175 | 0.005332 |
| | | elongin A) | | |
| 206707_x_at | C6orf32 | chromosome 6 open reading frame 32 | 0.971474 | 0.000624 |
| 207902_at | IL5RA | interleukin 5 receptor, alpha | 0.971188 | 3.52E−05 |
| 207026_s_at | ATP2B3 | ATPase, Ca++ transporting, plasma membrane 3 | 0.969925 | 2.38E−05 |
| 220288_at | MYO15A | myosin XVA | 0.969919 | 0.000667 |
| 202509_s_at | TNFAIP2 | tumor necrosis factor, alpha-induced protein 2 | 0.969386 | 5.70E−05 |
| 208053_at | GUCY2F | guanylate cyclase 2F, retinal | 0.969147 | 3.38E−07 |
| 220611_at | DAB1 | disabled homolog 1 (Drosophila) | 0.969066 | 0.000388 |
| 219937_at | TRHDE | thyrotropin-releasing hormone degrading enzyme | 0.968986 | 9.74E−08 |
| 215761_at | DMXL2 | Dmx-like 2 | 0.968794 | 3.38E−06 |
| 219994_at | APBB1IP | amyloid beta (A4) precursor protein-binding, family B, member 1 | 0.968502 | 1.25E−07 |
| | | interacting protein | | |
| 206367_at | REN | renin | 0.968477 | 0.00119 |
| 221022_s_at | PMFBP1 | polyamine modulated factor 1 binding protein 1 /// polyamine | 0.968236 | 3.56E−05 |
| | | modulated factor 1 binding protein 1 | | |
| 213994_s_at | SPON1 | spondin 1, extracellular matrix protein | 0.968006 | 4.23E−06 |
| 214558_at | GPR12 | G protein-coupled receptor 12 | 0.964812 | 0.001611 |
| 211438_at | TRHR | thyrotropin-releasing hormone receptor | 0.963805 | 7.84E−05 |
| 216911_s_at | HIC2 | hypermethylated in cancer 2 | 0.963379 | 8.08E−05 |
| 207359_at | CAMKK2 | calcium/calmodulin-dependent protein kinase kinase 2, beta | 0.962789 | 3.20E−05 |
| 203190_at | NDUFS8 | NADH dehydrogenase (ubiquinone) Fe—S protein 8, 23 kDa | 0.962197 | 0.004504 |
| | | (NADH-coenzyme Q reductase) | | |
| 220196_at | MUC16 | mucin 16 | 0.962065 | 6.60E−06 |
| 207408_at | SLC22A14 | solute carrier family 22 (organic cation transporter), member 14 | 0.961864 | 0.00172 |
| 217095_x_at | NCR1 | natural cytotoxicity triggering receptor 1 | 0.961826 | 0.001417 |
| 203332_s_at | INPP5D | inositol polyphosphate-5-phosphatase, 145 kDa | 0.961279 | 0.00864 |
| 220918_at | C21orf96 | chromosome 21 open reading frame 96 | 0.961257 | 0.000842 |
| 221948_s_at | KELCHL | Kelch-like 22 (Drosophila) | 0.960731 | 0.000541 |
| 212822_at | HEG1 | HEG homolog 1 (zebrafish) | 0.960187 | 3.48E−06 |
| 219781_s_at | LOC51333 | mesenchymal stem cell protein DSC43 | 0.960093 | 6.58E−05 |
| 212161_at | AP2A2 | adaptor-related protein complex 2, alpha 2 subunit | 0.959755 | 0.004453 |
| 53968_at | KIAA1698 | KIAA1698 protein | 0.958987 | 0.001682 |
| 206630_at | TYR | tyrosinase (oculocutaneous albinism IA) | 0.95882 | 0.000248 |
| 207890_s_at | MMP25 | matrix metallopeptidase 25 | 0.958581 | 0.002151 |
| 205033_s_at | DEFA1 /// DEFA3 | defensin, alpha 1 /// defensin, alpha 3, neutrophil-specific | 0.958401 | 5.03E−07 |
| 205504_at | BTK | Bruton agammaglobulinemia tyrosine kinase | 0.958311 | 0.000679 |
| 208574_at | SOX14 | SRY (sex determining region Y)-box 14 | 0.957942 | 0.000388 |
| 221099_at | — | — | 0.957269 | 0.001654 |
| 211307_s_at | FCAR | Fc fragment of IgA, receptor for | 0.956468 | 6.73E−06 |
| 214447_at | ETS1 | v-ets erythroblastosis virus E26 oncogene homolog 1 (avian) | 0.955524 | 0.000177 |
| 220743_at | — | — | 0.955227 | 0.000285 |
| 201063_at | RCN1 | reticulocalbin 1, EF-hand calcium binding domain | 0.954974 | 0.008775 |
| 204588_s_at | SLC7A7 | solute carrier family 7 (cationic amino acid transporter, y+ | 0.954551 | 0.000692 |
| | | system), member 7 | | |
| 216507_at | FLJ90013 | Hypothetical protein FLJ90013 | 0.954496 | 8.96E−09 |
| 203539_s_at | CHN2 | chimerin (chimaerin) 2 | 0.953171 | 3.98E−05 |
| 220309_at | TTC22 | tetratricopeptide repeat domain 22 | 0.951776 | 0.003222 |
| 217365_at | PRAMEF11 | PRAME family member 11 | 0.951383 | 0.000119 |
| 219720_s_at | C14orf118 | chromosome 14 open reading frame 118 | 0.951278 | 0.000245 |
| 214506_at | ADMR | adrenomedullin receptor | 0.95117 | 4.74E−06 |
| 213945_s_at | — | — | 0.950091 | 0.000512 |
| 221908_at | FLJ14627 | Hypothetical protein FLJ14627 | 0.950037 | 4.77E−06 |
| 220795_s_at | KIAA1446 | likely ortholog of rat brain-enriched guanylate kinase-associated | 0.949336 | 0.000245 |
| | | protein | | |
| 215126_at | — | CDNA FLJ42949 fis, clone BRSTN2006583 | 0.948775 | 0.000922 |
| 206508_at | TNFSF7 | tumor necrosis factor (ligand) superfamily, member 7 | 0.948023 | 2.94E−06 |
| 208300_at | PTPRH | protein tyrosine phosphatase, receptor type, H | 0.947916 | 3.23E−06 |
| 208191_x_at | PSG4 | pregnancy specific beta-1-glycoprotein 4 | 0.947811 | 0.001919 |
| 219243_at | GIMAP4 | GTPase, IMAP family member 4 | 0.947322 | 0.004675 |
| 214344_at | LOC92973 | hypothetical protein LOC92973 | 0.94722 | 0.000315 |
| 211499_s_at | MAPK11 | mitogen-activated protein kinase 11 | 0.946144 | 0.000412 |
| 214376_at | — | Clone 24626 mRNA sequence | 0.94599 | 0.000136 |
| 210223_s_at | MR1 | major histocompatibility complex, class I-related | 0.945776 | 4.67E−05 |
| 221240_s_at | B3GNT4 | UDP-GlcNAc:betaGal beta-1,3-N-acetylglucosaminyltransferase | 0.943408 | 0.001771 |
| | | 4 /// UDP-GlcNAc:betaGal beta-1,3-N- | | |
| | | acetylglucosaminyltransferase 4 | | |
| 213045_at | MAST3 | microtubule associated serine/threonine kinase 3 | 0.943103 | 0.000934 |
| 217120_s_at | CRSP2 | cofactor required for Sp1 transcriptional activation, subunit 2, | 0.943059 | 2.81E−08 |
| | | 150 kDa | | |
| 210369_at | SWAP70 | SWAP-70 protein | 0.942556 | 0.001198 |
| 210325_at | CD1A | CD1a antigen | 0.942355 | 2.44E−05 |
| 218660_at | DYSF | dysferlin, limb girdle muscular dystrophy 2B (autosomal | 0.941437 | 4.46E−05 |
| | | recessive) | | |
| 207919_at | ART1 | ADP-ribosyltransferase 1 | 0.941168 | 0.003683 |
| 210140_at | CST7 | cystatin F (leukocystatin) | 0.940948 | 0.000868 |
| 222187_x_at | G3BP | Ras-GTPase-activating protein SH3-domain-binding protein | 0.940886 | 0.002833 |
| 207537_at | PFKFB1 | 6-phosphofructo-2-kinase/fructose-2,6-biphosphatase 1 | 0.940421 | 0.000124 |
| 204016_at | LARS2 | leucyl-tRNA synthetase 2, mitochondrial | 0.939642 | 0.002428 |
| 205874_at | ITPKA | inositol 1,4,5-trisphosphate 3-kinase A | 0.939383 | 0.0017 |
| 210060_at | PDE6G | phosphodiesterase 6G, cGMP-specific, rod, gamma | 0.939259 | 0.000417 |
| 205456_at | CD3E | CD3E antigen, epsilon polypeptide (TiT3 complex) | 0.938427 | 0.003697 |
| 204896_s_at | PTGER4 | prostaglandin E receptor 4 (subtype EP4) | 0.938419 | 0.000833 |
| 219699_at | LGI2 | leucine-rich repeat LGI family, member 2 | 0.936908 | 0.00089 |
| 211102_s_at | LILRA2 | leukocyte immunoglobulin-like receptor, subfamily A (with TM | 0.935461 | 1.89E−05 |
| | | domain), member 2 | | |
| 206168_at | ZC3H7B | zinc finger CCCH-type containing 7B | 0.935152 | 0.00403 |
| 220524_at | EPB41L4B | erythrocyte membrane protein band 4.1 like 4B | 0.93495 | 6.07E−05 |
| 216501_at | VAC14 | Vac14 homolog (S. cerevisiae) | 0.934679 | 0.000341 |
| 214609_at | PHOX2A | paired-like (aristaless) homeobox 2a | 0.934516 | 0.00064 |
| 209827_s_at | IL16 | interleukin 16 (lymphocyte chemoattractant factor) | 0.934203 | 0.000219 |
| 220213_at | ZNF218 | zinc finger protein 218 | 0.934187 | 0.009603 |
| 206598_at | INS | insulin | 0.933682 | 0.000163 |
| 218409_s_at | DNAJC1 | DnaJ (Hsp40) homolog, subfamily C, member 1 | 0.933182 | 0.002171 |
| 40472_at | LOC254531 | PLSC domain containing protein | 0.932897 | 0.007037 |
| 208315_x_at | TRAF3 | TNF receptor-associated factor 3 | 0.932159 | 9.75E−07 |
| 218702_at | SARS2 | seryl-tRNA synthetase 2 | 0.932111 | 0.001478 |
| 213733_at | MYO1F | myosin IF | 0.931715 | 0.005239 |
| 202759_s_at | PALM2-AKAP2 | PALM2-AKAP2 protein | 0.931709 | 0.006223 |
| 221148_at | — | — | 0.931128 | 0.001105 |
| 205291_at | IL2RB | interleukin 2 receptor, beta /// interleukin 2 receptor, beta | 0.929637 | 0.003666 |
| 220792_at | PRDM5 | PR domain containing 5 | 0.929591 | 7.18E−05 |
| 218384_at | CARHSP1 | calcium regulated heat stable protein 1, 24 kDa | 0.929406 | 9.38E−05 |
| 210064_s_at | UPK1B | uroplakin 1B | 0.929291 | 0.001785 |
| 214443_at | PVR | poliovirus receptor | 0.929274 | 3.00E−06 |
| 221162_at | HHLA1 | HERV-H LTR-associating 1 | 0.929239 | 0.002818 |
| 209913_x_at | — | — | 0.929173 | 0.007018 |
| 207330_at | PZP | pregnancy-zone protein | 0.928844 | 0.00035 |
| 209851_at | KIAA0853 | KIAA0853 | 0.926285 | 0.00018 |
| 211262_at | PCSK6 | proprotein convertase subtilisin/kexin type 6 | 0.926089 | 0.001872 |
| 207066_at | HRC | histidine rich calcium binding protein | 0.925906 | 0.000666 |
| 211380_s_at | PRKG1 | protein kinase, cGMP-dependent, type I | 0.924419 | 0.000129 |
| 221629_x_at | C8orf30A | chromosome 8 open reading frame 30A | 0.923667 | 0.006093 |
| 1405_i_at | CCL5 | chemokine (C-C motif) ligand 5 | 0.922542 | 0.009512 |
| 206793_at | PNMT | phenylethanolamine N-methyltransferase | 0.922358 | 2.42E−05 |
| 221828_s_at | C9orf28 | chromosome 9 open reading frame 28 | 0.922291 | 0.001901 |
| 214863_at | — | Full length insert cDNA clone ZC35F11 | 0.921942 | 2.04E−06 |
| 215484_at | TRIM3 | Tripartite motif-containing 3 | 0.920328 | 0.005954 |
| 216082_at | NEU3 | Sialidase 3 (membrane sialidase) | 0.920173 | 0.002161 |
| 207915_at | — | — | 0.919943 | 7.38E−05 |
| 214925_s_at | SPTAN1 | spectrin, alpha, non-erythrocytic 1 (alpha-fodrin) | 0.919839 | 0.000206 |
| 208463_at | GABRA4 | gamma-aminobutyric acid (GABA) A receptor, alpha 4 | 0.919755 | 0.000125 |
| 208429_x_at | HNF4A | hepatocyte nuclear factor 4, alpha | 0.918856 | 0.004035 |
| 206340_at | NR1H4 | nuclear receptor subfamily 1, group H, member 4 | 0.918757 | 0.001697 |
| 64942_at | GPR153 | G protein-coupled receptor 153 | 0.918574 | 8.95E−05 |
| 206625_at | RDS | retinal degeneration, slow | 0.918354 | 2.14E−05 |
| 210135_s_at | SHOX2 | short stature homeobox 2 | 0.917223 | 3.89E−07 |
| 206800_at | MTHFR | 5,10-methylenetetrahydrofolate reductase (NADPH) | 0.917052 | 0.00011 |
| 219838_at | TTC23 | tetratricopeptide repeat domain 23 | 0.916964 | 0.001938 |
| 212707_s_at | RASA4 /// | RAS p21 protein activator 4 /// hypothetical protein FLJ21767 | 0.916446 | 0.004167 |
| | FLJ21767 | | | |
| 208126_s_at | CYP2C18 | cytochrome P450, family 2, subfamily C, polypeptide 18 /// | 0.915609 | 7.20E−05 |
| | | cytochrome P450, family 2, subfamily C, polypeptide 18 | | |
| 210258_at | RGS13 | regulator of G-protein signalling 13 | 0.915366 | 8.42E−08 |
| 211815_s_at | GGA3 | golgi associated, gamma adaptin ear containing, ARF binding | 0.915231 | 0.007716 |
| | | protein 3 | | |
| 206123_at | LLGL1 | lethal giant larvae homolog 1 (Drosophila) | 0.91445 | 0.001826 |
| 206663_at | SP4 | Sp4 transcription factor | 0.914144 | 9.78E−05 |
| 209997_x_at | PCM1 | pericentriolar material 1 | 0.913982 | 8.80E−05 |
| 205106_at | MTCP1 | mature T-cell proliferation 1 | 0.913926 | 0.000114 |
| 205403_at | IL1R2 | interleukin 1 receptor, type II | 0.913454 | 0.008767 |
| 216514_at | — | — | 0.912848 | 0.000234 |
| 205182_s_at | ZNF324 | zinc finger protein 324 | 0.912844 | 0.003284 |
| 205524_s_at | HAPLN1 | hyaluronan and proteoglycan link protein 1 | 0.911608 | 0.00132 |
| 206191_at | ENTPD3 | ectonucleoside triphosphate diphosphohydrolase 3 | 0.91112 | 0.007054 |
| 210442_at | IL1RL1 | interleukin 1 receptor-like 1 | 0.911106 | 0.000988 |
| 208439_s_at | FCN2 | ficolin (collagen/fibrinogen domain containing lectin) 2 (hucolin) | 0.910393 | 0.002278 |
| 205639_at | AOAH | acyloxyacyl hydrolase (neutrophil) | 0.910339 | 0.001896 |
| 207395_at | BTN1A1 | butyrophilin, subfamily 1, member A1 | 0.909998 | 0.000847 |
| 221820_s_at | MYST1 | MYST histone acetyltransferase 1 | 0.909873 | 9.13E−06 |
| 210080_x_at | ELA3A | elastase 3A, pancreatic | 0.909449 | 0.003787 |
| 211389_x_at | KIR3DL1 | killer cell immunoglobulin-like receptor, three domains, long | 0.909362 | 0.00063 |
| | | cytoplasmic tail, 1 | | |
| 203103_s_at | PRPF19 | PRP19/PSO4 pre-mRNA processing factor 19 homolog (S. cerevisiae) | 0.909287 | 0.000708 |
| 220826_at | C21orf77 | chromosome 21 open reading frame 77 | 0.907489 | 5.77E−05 |
| 222346_at | LAMA1 | laminin, alpha 1 | 0.905613 | 0.005743 |
| 210441_at | CDC14A | CDC14 cell division cycle 14 homolog A (S. cerevisiae) | 0.905417 | 3.47E−06 |
| 220815_at | CTNNA3 | catenin (cadherin-associated protein), alpha 3 | 0.904306 | 0.00121 |
| 201884_at | CEACAM5 | carcinoembryonic antigen-related cell adhesion molecule 5 | 0.903545 | 0.007521 |
| 210617_at | PHEX | phosphate regulating endopeptidase homolog, X-linked | 0.9035 | 0.004225 |
| | | (hypophosphatemia, vitamin D resistant rickets) | | |
| 207167_at | IGSF2 | immunoglobulin superfamily, member 2 | 0.902945 | 1.20E−06 |
| 209951_s_at | MAP2K7 | mitogen-activated protein kinase kinase 7 | 0.901329 | 0.002096 |
| 213917_at | — | — | 0.900237 | 2.62E−06 |
| 201265_at | — | — | 0.899375 | 0.006351 |
| 205929_at | GPA33 | glycoprotein A33 (transmembrane) | 0.899265 | 0.003078 |
| 207075_at | CIAS1 | cold autoinflammatory syndrome 1 | 0.899193 | 0.000191 |
| 220246_at | CAMK1D | calcium/calmodulin-dependent protein kinase ID | 0.898767 | 0.004189 |
| 210843_s_at | MFAP3L | microfibrillar-associated protein 3-like | 0.898522 | 0.000468 |
| 206531_at | DPF1 | D4, zinc and double PHD fingers family 1 | 0.898467 | 0.000134 |
| 217728_at | S100A6 | S100 calcium binding protein A6 (calcyclin) | 0.898182 | 0.00582 |
| 64440_at | IL17RC | interleukin 17 receptor C | 0.897985 | 0.000724 |
| 220571_at | PRDM11 | PR domain containing 11 | 0.897562 | 0.001026 |
| 207353_s_at | HMX1 | homeo box (H6 family) 1 | 0.897475 | 0.00024 |
| 217473_x_at | SLC11A1 | solute carrier family 11 (proton-coupled divalent metal ion | 0.897153 | 8.45E−06 |
| | | transporters), member 1 | | |
| 206486_at | LAG3 | lymphocyte-activation gene 3 | 0.897007 | 0.000625 |
| 207568_at | CHRNA6 | cholinergic receptor, nicotinic, alpha polypeptide 6 | 0.896591 | 7.49E−05 |
| 211372_s_at | IL1R2 | interleukin 1 receptor, type II | 0.896299 | 4.16E−09 |
| 213085_s_at | KIBRA | KIBRA protein | 0.896232 | 0.003845 |
| 216354_at | — | — | 0.896107 | 0.000145 |
| 216849_at | FLJ16124 | FLJ16124 protein | 0.895984 | 0.000779 |
| 213820_s_at | STARD5 | START domain containing 5 | 0.89595 | 0.000457 |
| 212045_at | GLG1 | golgi apparatus protein 1 | 0.895905 | 8.27E−05 |
| 221724_s_at | CLEC4A | C-type lectin domain family 4, member A | 0.89571 | 0.000548 |
| 217261_at | TTTY2 | testis-specific transcript, Y-linked 2 | 0.895076 | 3.98E−05 |
| 214406_s_at | SLC7A4 | solute carrier family 7 (cationic amino acid transporter, y+ | 0.894468 | 4.20E−05 |
| | | system), member 4 | | |
| 1431_at | CYP2E1 | cytochrome P450, family 2, subfamily E, polypeptide 1 | 0.893819 | 8.64E−09 |
| 207501_s_at | FGF12 | fibroblast growth factor 12 | 0.892461 | 9.33E−06 |
| 205233_s_at | PAFAH2 | platelet-activating factor acetylhydrolase 2, 40 kDa | 0.892418 | 0.003492 |
| 208417_at | FGF6 | fibroblast growth factor 6 | 0.891957 | 2.75E−05 |
| 218742_at | NARFL | nuclear prelamin A recognition factor-like | 0.891804 | 0.001888 |
| 221042_s_at | CLMN | calmin (calponin-like, transmembrane) | 0.891435 | 0.00507 |
| 216678_at | WDR10 | WD repeat domain 10 | 0.890952 | 0.000611 |
| 211902_x_at | TRA@ | T cell receptor alpha locus | 0.890799 | 0.000465 |
| 219692_at | KREMEN2 | kringle containing transmembrane protein 2 | 0.890418 | 0.000429 |
| 211395_x_at | FCGR2C | Fc fragment of IgG, low affinity IIc, receptor for (CD32) | 0.89012 | 0.009727 |
| 216376_x_at | — | — | 0.889859 | 0.00407 |
| 207360_s_at | NTSR1 | neurotensin receptor 1 (high affinity) | 0.888996 | 0.000173 |
| 205175_s_at | KHK | ketohexokinase (fructokinase) | 0.88859 | 0.000288 |
| 179_at | PMS2L11 | postmeiotic segregation increased 2-like 11 | 0.88773 | 0.001602 |
| 222058_at | RNF130 | ring finger protein 130 | 0.887109 | 0.004514 |
| 203331_s_at | INPP5D | inositol polyphosphate-5-phosphatase, 145 kDa | 0.886934 | 0.000161 |
| 218584_at | FLJ21127 | hypothetical protein FLJ21127 | 0.886789 | 0.000317 |
| 211189_x_at | CD84 | CD84 antigen (leukocyte antigen) | 0.886564 | 0.000311 |
| 216055_at | PDGFB | Platelet-derived growth factor beta polypeptide (simian sarcoma | 0.88617 | 0.000195 |
| | | viral (v-sis) oncogene homolog) | | |
| 217158_at | LOC401525 | similar to tumor suppressor deleted in oral cancer-related 1 | 0.886142 | 0.001433 |
| 216584_at | — | — | 0.885923 | 0.001746 |
| 210838_s_at | ACVRL1 | activin A receptor type II-like 1 | 0.885889 | 4.69E−07 |
| 210461_s_at | ABLIM1 | actin binding LIM protein 1 | 0.885788 | 0.000383 |
| 216909_at | KIAA0690 | KIAA0690 | 0.885774 | 4.81E−05 |
| 221997_s_at | MRPL52 | Mitochondrial ribosomal protein L52 | 0.885656 | 0.000125 |
| 209407_s_at | DEAF1 | deformed epidermal autoregulatory factor 1 (Drosophila) | 0.885363 | 0.009904 |
| 205790_at | SCAP1 | src family associated phosphoprotein 1 | 0.885175 | 0.000807 |
| 203888_at | THBD | thrombomodulin | 0.884706 | 0.005264 |
| 216912_at | ARHGEF4 | Rho guanine nucleotide exchange factor (GEF) 4 | 0.884548 | 6.04E−05 |
| 214301_s_at | DPYSL4 | Dihydropyrimidinase-like 4 | 0.884202 | 0.004768 |
| 208242_at | RAX | retina and anterior neural fold homeobox | 0.884195 | 0.002392 |
| 216356_x_at | BAIAP3 | BAI1-associated protein 3 | 0.882958 | 0.007904 |
| 207258_at | DSCR4 | Down syndrome critical region gene 4 | 0.882771 | 0.001306 |
| 203441_s_at | CDH2 | cadherin 2, type 1, N-cadherin (neuronal) | 0.882416 | 0.001164 |
| 221080_s_at | DENND1C | DENN/MADD domain containing 1C | 0.882251 | 1.72E−06 |
| 220476_s_at | C1orf183 | chromosome 1 open reading frame 183 | 0.881818 | 0.00247 |
| 214969_at | MAP3K9 | mitogen-activated protein kinase kinase kinase 9 | 0.881787 | 0.000995 |
| 65585_at | FAM86B1 | family with sequence similarity 86, member B1 | 0.881319 | 0.005841 |
| 216540_at | — | TCR V alpha 14.1/J alpha 32/C alpha = melanoma antigen- | 0.881124 | 0.005302 |
| | | specific T-cell receptor alpha chain {V-J-C junction, alternatively | | |
| | | spliced, clone TIL C10-1} [human, CD8+ tumor-infiltrating | | |
| | | lymphocytes, mRNA Partial, 108 nt] | | |
| 202473_x_at | HCFC1 | host cell factor C1 (VP16-accessory protein) | 0.880976 | 0.000185 |
| 221859_at | SYT13 | synaptotagmin XIII | 0.880516 | 0.004475 |
| 211441_x_at | CYP3A43 | cytochrome P450, family 3, subfamily A, polypeptide 43 | 0.879752 | 0.000585 |
| 208260_at | AVPR1B | arginine vasopressin receptor 1B | 0.879447 | 0.004078 |
| 206325_at | SERPINA6 | serpin peptidase inhibitor, clade A (alpha-1 antiproteinase, | 0.879177 | 2.10E−05 |
| | | antitrypsin), member 6 | | |
| 214070_s_at | ATP10B | ATPase, Class V, type 10B | 0.878654 | 0.00107 |
| 204138_s_at | ZNF42 | zinc finger protein 42 (myeloid-specific retinoic acid-responsive) | 0.878602 | 0.00105 |
| 207701_at | C22orf24 | chromosome 22 open reading frame 24 | 0.878251 | 0.003312 |
| 206617_s_at | RENBP | renin binding protein | 0.877962 | 0.006423 |
| 217629_at | — | Transcribed locus, weakly similar to XP_498467.1 PREDICTED: | 0.877538 | 0.000221 |
| | | hypothetical protein XP_498467 [Homo sapiens] | | |
| 216285_at | DGCR14 | DiGeorge syndrome critical region gene 14 | 0.876436 | 2.11E−05 |
| 212873_at | HA-1 | minor histocompatibility antigen HA-1 | 0.876282 | 0.006107 |
| 213113_s_at | SLC43A3 | solute carrier family 43, member 3 | 0.87567 | 0.000886 |
| 216865_at | COL14A1 | collagen, type XIV, alpha 1 (undulin) | 0.875622 | 6.83E−09 |
| 214197_s_at | SETDB1 | SET domain, bifurcated 1 | 0.874204 | 0.002121 |
| 220032_at | FLJ21986 | hypothetical protein FLJ21986 | 0.874084 | 0.002551 |
| 211794_at | FYB | FYN binding protein (FYB-120/130) | 0.873983 | 1.02E−05 |
| 214205_x_at | TXNL2 | thioredoxin-like 2 | 0.87394 | 0.005241 |
| 204462_s_at | SLC16A2 | solute carrier family 16 (monocarboxylic acid transporters), | 0.873773 | 4.73E−05 |
| | | member 2 | | |
| 203920_at | NR1H3 | nuclear receptor subfamily 1, group H, member 3 | 0.872932 | 0.001946 |
| 220873_at | REPS2 | RALBP1 associated Eps domain containing 2 | 0.87271 | 4.44E−05 |
| 214391_x_at | PTGER1 | prostaglandin E receptor 1 (subtype EP1), 42 kDa | 0.872005 | 2.05E−05 |
| 213484_at | — | Clone 23700 mRNA sequence | 0.871888 | 0.0054 |
| 205150_s_at | KIAA0644 | KIAA0644 gene product | 0.87179 | 0.00065 |
| 221313_at | GPR52 | G protein-coupled receptor 52 | 0.871711 | 0.00133 |
| 221250_s_at | MXD3 | MAX dimerization protein 3 /// MAX dimerization protein 3 | 0.871569 | 0.001593 |
| 216688_at | — | MRNA; cDNA DKFZp434C2331 (from clone DKFZp434C2331) | 0.871559 | 0.002427 |
| 208287_at | HCG9 | HLA complex group 9 | 0.871307 | 3.03E−06 |
| 216330_s_at | POU6F1 | POU domain, class 6, transcription factor 1 | 0.870981 | 0.00084 |
| 211590_x_at | TBXA2R | thromboxane A2 receptor | 0.870258 | 0.00259 |
| 205801_s_at | RASGRP3 | RAS guanyl releasing protein 3 (calcium and DAG-regulated) | 0.869696 | 6.14E−06 |
| 219784_at | FBXO31 | F-box protein 31 | 0.869542 | 4.76E−05 |
| 204038_s_at | EDG2 | endothelial differentiation, lysophosphatidic acid G-protein- | 0.869435 | 0.001318 |
| | | coupled receptor, 2 | | |
| 39763_at | HPX | hemopexin | 0.869381 | 0.000679 |
| 215697_at | KIAA0318 | RIM binding protein 2 | 0.868829 | 0.004419 |
| 210865_at | FASLG | Fas ligand (TNF superfamily, member 6) | 0.868775 | 0.000269 |
| 220977_x_at | EPB41L5 | erythrocyte membrane protein band 4.1 like 5 /// erythrocyte | 0.86835 | 0.001454 |
| | | membrane protein band 4.1 like 5 | | |
| 220514_at | NAP1L4 | Nucleosome assembly protein 1-like 4 | 0.867935 | 2.69E−05 |
| 204002_s_at | ICA1 | islet cell autoantigen 1, 69 kDa | 0.867793 | 0.006207 |
| 208303_s_at | CRLF2 | cytokine receptor-like factor 2 | 0.867666 | 0.000715 |
| 202999_s_at | LOXL2 | lysyl oxidase-like 2 | 0.867293 | 0.003275 |
| 207955_at | CCL27 | chemokine (C-C motif) ligand 27 | 0.86648 | 0.00029 |
| 214926_at | SPTAN1 | spectrin, alpha, non-erythrocytic 1 (alpha-fodrin) | 0.865929 | 1.02E−05 |
| 206602_s_at | HOXD3 | homeo box D3 | 0.865777 | 0.000871 |
| 221187_s_at | FLJ22688 | hypothetical protein FLJ22688 | 0.86524 | 0.007743 |
| 221754_s_at | CORO1B | coronin, actin binding protein, 1B | 0.86466 | 0.001857 |
| 219579_at | RAB3IL1 | RAB3A interacting protein (rabin3)-like 1 | 0.864289 | 0.003327 |
| 207463_x_at | PRSS3 | protease, serine, 3 (mesotrypsin) | 0.863898 | 2.23E−05 |
| 52940_at | SIGIRR | single immunoglobulin and toll-interleukin 1 receptor (TIR) | 0.863651 | 0.000105 |
| | | domain | | |
| 202252_at | RAB13 | RAB13, member RAS oncogene family | 0.863116 | 0.003683 |
| 217032_at | FOXD4L1 | forkhead box D4-like 1 | 0.862573 | 0.003821 |
| 207432_at | VMD2L1 | vitelliform macular dystrophy 2-like 1 | 0.862103 | 0.0077 |
| 205031_at | EFNB3 | ephrin-B3 | 0.861805 | 0.001803 |
| 208807_s_at | CHD3 | chromodomain helicase DNA binding protein 3 | 0.861487 | 0.000109 |
| 204893_s_at | ZFYVE9 | zinc finger, FYVE domain containing 9 | 0.860821 | 0.004671 |
| 215654_at | BCAT2 | branched chain aminotransferase 2, mitochondrial | 0.860771 | 0.004391 |
| 216824_at | LOC442334 /// | ADP-ribosylation factor 1 pseudogene /// similar to dJ133P16.1 | 0.860345 | 0.003856 |
| | LOC442711 | (ADP-ribosylation factor 1) | | |
| 205797_s_at | TCP11L1 | t-complex 11 (mouse) like 1 | 0.860241 | 7.53E−06 |
| 215003_at | DGCR9 | DiGeorge syndrome critical region gene 9 | 0.860115 | 0.001958 |
| 218921_at | SIGIRR | single immunoglobulin and toll-interleukin 1 receptor (TIR) | 0.859653 | 0.009406 |
| | | domain | | |
| 207637_at | KIAA0789 | KIAA0789 gene product | 0.858257 | 0.000183 |
| 216750_at | APBB2 | amyloid beta (A4) precursor protein-binding, family B, member 2 | 0.858248 | 7.16E−05 |
| | | (Fe65-like) | | |
| 220875_at | — | — | 0.858226 | 0.000393 |
| 204701_s_at | STOML1 | stomatin (EPB72)-like 1 | 0.858146 | 0.001115 |
| 206199_at | CEACAM7 | carcinoembryonic antigen-related cell adhesion molecule 7 | 0.857617 | 0.002432 |
| 220563_s_at | SHANK1 | SH3 and multiple ankyrin repeat domains 1 | 0.856415 | 3.12E−05 |
| 220439_at | RIN3 | Ras and Rab interactor 3 | 0.856404 | 0.004883 |
| 206807_s_at | ADD2 | adducin 2 (beta) | 0.856229 | 0.004691 |
| 211118_x_at | ESR2 | estrogen receptor 2 (ER beta) | 0.856026 | 9.23E−05 |
| 210798_x_at | MASP2 | mannan-binding lectin serine peptidase 2 | 0.855827 | 0.000537 |
| 221354_s_at | GPR24 | G protein-coupled receptor 24 | 0.855768 | 1.51E−05 |
| 216371_at | — | — | 0.855255 | 0.00016 |
| 210651_s_at | EPHB2 | EPH receptor B2 | 0.854712 | 0.002339 |
| 204684_at | NPTX1 | neuronal pentraxin I | 0.854664 | 0.00138 |
| 212524_x_at | H2AFX | H2A histone family, member X | 0.854261 | 0.000333 |
| 205988_at | CD84 | CD84 antigen (leukocyte antigen) | 0.854212 | 0.006972 |
| 213003_s_at | — | — | 0.853995 | 0.005828 |
| 211884_s_at | CIITA | class II, major histocompatibility complex, transactivator | 0.853094 | 8.89E−05 |
| 202107_s_at | MCM2 | MCM2 minichromosome maintenance deficient 2, mitotin (S. cerevisiae) | 0.852926 | 0.00294 |
| 216757_at | GALNT7 | UDP-N-acetyl-alpha-D-galactosamine:polypeptide N- | 0.852494 | 0.002395 |
| | | acetylgalactosaminyltransferase 7 (GalNAc-T7) | | |
| 216346_at | SEC14L3 | SEC14-like 3 (S. cerevisiae) | 0.851003 | 4.87E−05 |
| 215295_at | DTNB | dystrobrevin, beta | 0.850667 | 6.22E−07 |
| 219246_s_at | FLJ13491 | hypothetical protein FLJ13491 | 0.850492 | 0.003471 |
| 38710_at | OTUB1 | OTU domain, ubiquitin aldehyde binding 1 | 0.85044 | 0.000192 |
| 207477_at | — | — | 0.850423 | 0.00288 |
| 208285_at | OR7A5 | olfactory receptor, family 7, subfamily A, member 5 | 0.850122 | 0.008317 |
| 210663_s_at | KYNU | kynureninase (L-kynurenine hydrolase) | 0.8498 | 5.42E−06 |
| 208559_at | IPF1 | insulin promoter factor 1, homeodomain transcription factor | 0.849776 | 0.000599 |
| 219610_at | RGNEF | Rho-guanine nucleotide exchange factor | 0.849436 | 0.004942 |
| 207864_at | SCN7A | sodium channel, voltage-gated, type VII, alpha | 0.848979 | 0.000165 |
| 221140_s_at | GPR132 | G protein-coupled receptor 132 | 0.848849 | 1.80E−05 |
| 219457_s_at | RIN3 | Ras and Rab interactor 3 | 0.848234 | 0.000158 |
| 204272_at | LGALS4 | lectin, galactoside-binding, soluble, 4 (galectin 4) | 0.848178 | 0.003667 |
| 217150_s_at | NF2 | neurofibromin 2 (bilateral acoustic neuroma) | 0.847791 | 0.005105 |
| 216168_at | RHOH | Ras homolog gene family, member H | 0.847527 | 0.000364 |
| 91920_at | BCAN | brevican | 0.846724 | 0.001148 |
| 201596_x_at | KRT18 | keratin 18 | 0.846269 | 0.001081 |
| 206655_s_at | GP1BB | glycoprotein Ib (platelet), beta polypeptide | 0.845864 | 0.00953 |
| 38691_s_at | SFTPC | surfactant, pulmonary-associated protein C | 0.845532 | 0.009071 |
| 207459_x_at | GYPB | glycophorin B (includes Ss blood group) | 0.845525 | 0.004905 |
| 207351_s_at | SH2D2A | SH2 domain protein 2A | 0.845047 | 0.004523 |
| 206358_at | PRM1 | protamine 1 | 0.844166 | 3.18E−05 |
| 207349_s_at | UCP3 | uncoupling protein 3 (mitochondrial, proton carrier) | 0.843948 | 3.37E−06 |
| 220778_x_at | SEMA6B | sema domain, transmembrane domain (TM), and cytoplasmic | 0.843882 | 0.000109 |
| | | domain, (semaphorin) 6B | | |
| 216182_at | SYNJ2 | Synaptojanin 2 | 0.843175 | 0.001066 |
| 205982_x_at | SFTPC | surfactant, pulmonary-associated protein C | 0.842955 | 0.009112 |
| 216857_at | — | T-cell receptor active beta-chain (V10-D-J-C) mRNA, clone | 0.842781 | 0.002451 |
| | | PL3.9 | | |
| 218454_at | FLJ22662 | hypothetical protein FLJ22662 | 0.842399 | 0.002987 |
| 216921_s_at | KRTHA5 | keratin, hair, acidic, 5 | 0.842264 | 5.04E−05 |
| 213874_at | SERPINA4 | serpin peptidase inhibitor, clade A (alpha-1 antiproteinase, | 0.841801 | 0.00078 |
| | | antitrypsin), member 4 | | |
| AFFX- | — | — | 0.8415 | 0.001008 |
| M27830_3_at | | | | |
| 210677_at | SOAT2 | sterol O-acyltransferase 2 | 0.841056 | 0.000485 |
| 220107_s_at | C14orf140 | chromosome 14 open reading frame 140 | 0.840942 | 2.07E−05 |
| 206524_at | T | T, brachyury homolog (mouse) | 0.840398 | 0.002165 |
| 203317_at | PSD4 | pleckstrin and Sec7 domain containing 4 | 0.840178 | 0.008102 |
| 214192_at | RABEP1 | Rabaptin, RAB GTPase binding effector protein 1 | 0.840071 | 0.003721 |
| 204848_x_at | HBG1 | hemoglobin, gamma A /// hemoglobin, gamma A | 0.840017 | 0.002107 |
| 216705_s_at | ADA | adenosine deaminase | 0.839774 | 0.000153 |
| 205447_s_at | MAP3K12 | mitogen-activated protein kinase kinase kinase 12 | 0.839549 | 0.000975 |
| 216662_at | MYO7B | myosin VIIB | 0.839088 | 0.002118 |
| 205203_at | PLD1 | phospholipase D1, phophatidylcholine-specific | 0.838684 | 0.00398 |
| 205495_s_at | GNLY | granulysin /// granulysin | 0.838495 | 0.005164 |
| 206398_s_at | CD19 | CD19 antigen | 0.838434 | 3.81E−05 |
| 210506_at | FUT7 | fucosyltransferase 7 (alpha (1,3) fucosyltransferase) | 0.838299 | 0.000825 |
| 207206_s_at | ALOX12 | arachidonate 12-lipoxygenase | 0.838067 | 1.87E−05 |
| 205847_at | PRSS22 | protease, serine, 22 | 0.837831 | 0.0017 |
| 214490_at | ARSF | arylsulfatase F | 0.836557 | 0.001553 |
| 207697_x_at | LILRB2 | leukocyte immunoglobulin-like receptor, subfamily B (with TM | 0.835008 | 0.00753 |
| | | and ITIM domains), member 2 | | |
| 216993_s_at | COL11A2 | collagen, type XI, alpha 2 | 0.834892 | 0.003152 |
| 219718_at | FLJ10986 | hypothetical protein FLJ10986 | 0.834723 | 0.001015 |
| 211532_x_at | KIR2DS2 | killer cell immunoglobulin-like receptor, two domains, short | 0.8347 | 0.000112 |
| | | cytoplasmic tail, 2 | | |
| 217152_at | NCOR1 | Nuclear receptor co-repressor 1 | 0.834399 | 0.008458 |
| 204450_x_at | APOA1 /// | apolipoprotein A-I /// similar to Apolipoprotein A-I precursor (Apo- | 0.834267 | 0.000601 |
| | LOC440837 | AI) | | |
| 208346_at | PPBPL2 | pro-platelet basic protein-like 2 | 0.834157 | 2.48E−07 |
| 220460_at | SLCO1C1 | solute carrier organic anion transporter family, member 1C1 | 0.833961 | 0.000188 |
| 207925_at | CST5 | cystatin D | 0.83379 | 0.001118 |
| 222254_at | — | — | 0.8335 | 0.006338 |
| 205888_s_at | KIAA0555 | Jak and microtubule interacting protein 2 | 0.83333 | 5.55E−06 |
| 217222_at | V-set and | Similar to Ig heavy chain V-I region HG3 precursor | 0.833258 | 0.000354 |
| | immunoglobulin | | | |
| | domain | | | |
| | containing 6 | | | |
| 215413_at | EXOC7 | exocyst complex component 7 | 0.832972 | 0.001793 |
| 202837_at | TRAFD1 | TRAF-type zinc finger domain containing 1 | 0.832726 | 0.000737 |
| 208122_x_at | KIR2DS3 | killer cell immunoglobulin-like receptor, two domains, short | 0.832409 | 0.00342 |
| | | cytoplasmic tail, 3 | | |
| 213421_x_at | PRSS3 | protease, serine, 3 (mesotrypsin) | 0.832239 | 0.001226 |
| 220577_at | GVIN1 | GTPase, very large interferon inducible 1 | 0.832187 | 0.001014 |
| 217606_at | — | MRNA; cDNA DKFZp686P24158 (from clone DKFZp686P24158) | 0.83157 | 0.005491 |
| 200055_at | TAF10 | TAF10 RNA polymerase II, TATA box binding protein (TBP)- | 0.831507 | 0.000508 |
| | | associated factor, 30 kDa /// TAF10 RNA polymerase II, TATA | | |
| | | box binding protein (TBP)-associated factor, 30 kDa | | |
| 204397_at | EML2 | echinoderm microtubule associated protein like 2 | 0.831093 | 0.002218 |
| 221407_at | CX36 | connexin-36 | 0.830803 | 0.002076 |
| 205484_at | SIT1 | signaling threshold regulating transmembrane adaptor 1 /// | 0.830778 | 3.05E−05 |
| | | signaling threshold regulating transmembrane adaptor 1 | | |
| 217397_at | — | T cell receptor V alpha gene segment V-alpha-w24, clone | 0.830644 | 0.000102 |
| | | IGRa02 | | |
| 204874_x_at | BAIAP3 | BAI1-associated protein 3 | 0.830529 | 0.006974 |
| 206176_at | BMP6 | bone morphogenetic protein 6 | 0.829926 | 0.008036 |
| 218624_s_at | MGC2752 | hypothetical protein MGC2752 | 0.829469 | 0.006444 |
| 219745_at | C10orf77 | chromosome 10 open reading frame 77 | 0.827648 | 0.000216 |
| 210039_s_at | PRKCQ | protein kinase C, theta | 0.827445 | 0.001937 |
| 204419_x_at | HBG1 /// HBG2 | hemoglobin, gamma A /// hemoglobin, gamma A /// hemoglobin, | 0.827344 | 0.003002 |
| | | gamma G /// hemoglobin, gamma G | | |
| 206444_at | PDE1B | phosphodiesterase 1B, calmodulin-dependent | 0.827286 | 0.000697 |
| 203597_s_at | WBP4 | WW domain binding protein 4 (formin binding protein 21) | 0.827136 | 0.002215 |
| 216363_at | — | — | 0.827097 | 0.000529 |
| 202338_at | TK1 | thymidine kinase 1, soluble | 0.826562 | 0.000195 |
| 221032_s_at | TMPRSS5 | transmembrane protease, serine 5 (spinesin) /// transmembrane | 0.826283 | 0.000563 |
| | | protease, serine 5 (spinesin) | | |
| 209521_s_at | AMOT | angiomotin | 0.826042 | 5.88E−05 |
| 215712_s_at | IGFALS | insulin-like growth factor binding protein, acid labile subunit | 0.825709 | 0.000395 |
| 204845_s_at | ENPEP | glutamyl aminopeptidase (aminopeptidase A) | 0.825585 | 2.23E−06 |
| 215036_at | IGLC2 | Immunoglobulin lambda constant 1 (Mcg marker) | 0.824869 | 0.000167 |
| 207770_x_at | CSH2 | chorionic somatomammotropin hormone 2 | 0.824469 | 0.006528 |
| 216577_at | — | — | 0.824371 | 0.00566 |
| 217192_s_at | PRDM1 | PR domain containing 1, with ZNF domain | 0.824299 | 0.002806 |
| 216745_x_at | LRCH1 | Leucine-rich repeats and calponin homology (CH) domain | 0.824287 | 0.001504 |
| | | containing 1 | | |
| 207682_s_at | KIF25 | kinesin family member 25 | 0.824051 | 1.74E−05 |
| 221603_at | PEX16 | peroxisomal biogenesis factor 16 | 0.823451 | 0.004913 |
| 41856_at | UNC5B | Unc-5 homolog B (C. elegans) | 0.823341 | 0.000741 |
| 206703_at | CHRNB1 | cholinergic receptor, nicotinic, beta polypeptide 1 (muscle) | 0.823278 | 0.006994 |
| 214312_at | FOXA2 | Forkhead box A2 | 0.823255 | 0.001685 |
| 208221_s_at | SLIT1 | slit homolog 1 (Drosophila) | 0.823089 | 0.001039 |
| 204890_s_at | LCK | lymphocyte-specific protein tyrosine kinase | 0.823026 | 0.009338 |
| 211182_x_at | RUNX1 | runt-related transcription factor 1 (acute myeloid leukemia 1; | 0.822846 | 0.003972 |
| | | aml1 oncogene) | | |
| 214062_x_at | NFKBIB | nuclear factor of kappa light polypeptide gene enhancer in B- | 0.822183 | 0.003967 |
| | | cells inhibitor, beta | | |
| 217537_x_at | — | Transcribed locus, weakly similar to NP_055301.1 neuronal | 0.821786 | 0.000808 |
| | | thread protein AD7c-NTP [Homo sapiens] | | |
| 215688_at | RASGRF1 | Ras protein-specific guanine nucleotide-releasing factor 1 | 0.821194 | 6.10E−05 |
| 220628_s_at | SDK2 | sidekick homolog 2 (chicken) | 0.82084 | 0.000193 |
| 205101_at | CIITA | class II, major histocompatibility complex, transactivator | 0.820817 | 0.001039 |
| 215996_at | — | — | 0.820763 | 0.003043 |
| 206749_at | CD1B | CD1b antigen /// CD1b antigen | 0.820728 | 0.000449 |
| 200715_x_at | RPL13A | ribosomal protein L13a | 0.820563 | 0.000682 |
| 207146_at | KRTHA2 | keratin, hair, acidic, 2 | 0.820305 | 5.97E−05 |
| 215811_at | SNCA | Synuclein, alpha (non A4 component of amyloid precursor) | 0.819527 | 0.004545 |
| 214817_at | UNC13A | unc-13 homolog A (C. elegans) | 0.819064 | 2.41E−05 |
| 208267_at | TRPV5 | transient receptor potential cation channel, subfamily V, member 5 | 0.818969 | 2.12E−05 |
| 210822_at | LOC283345 | RPL13-2 pseudogene | 0.818945 | 0.000532 |
| 217453_at | — | MRNA; cDNA DKFZp434E2028 (from clone DKFZp434E2028) | 0.818861 | 0.001843 |
| 215485_s_at | ICAM1 | intercellular adhesion molecule 1 (CD54), human rhinovirus | 0.818667 | 0.000583 |
| | | receptor | | |
| 208179_x_at | KIR2DL3 | killer cell immunoglobulin-like receptor, two domains, long | 0.81765 | 1.50E−05 |
| | | cytoplasmic tail, 3 | | |
| 219884_at | LHX6 | LIM homeobox 6 | 0.81745 | 0.003771 |
| 220630_s_at | CHIA | chitinase, acidic | 0.817113 | 0.000965 |
| 204329_s_at | ZNF202 | zinc finger protein 202 | 0.816722 | 2.97E−05 |
| 207129_at | CA5B | carbonic anhydrase VB, mitochondrial | 0.816256 | 0.001177 |
| 205623_at | ALDH3A1 | aldehyde dehydrogenase 3 family, memberA1 | 0.815949 | 0.000173 |
| 210708_x_at | CASP10 | caspase 10, apoptosis-related cysteine peptidase | 0.815312 | 0.001296 |
| 222281_s_at | — | — | 0.815191 | 0.000175 |
| 204689_at | HHEX | hematopoietically expressed homeobox | 0.814661 | 0.000764 |
| 216409_at | ACSL6 | Acyl-CoA synthetase long-chain family member 6 | 0.81463 | 0.001007 |
| 214066_x_at | NPR2 | natriuretic peptide receptor B/guanylate cyclase B | 0.814606 | 3.98E−05 |
| | | (atrionatriuretic peptide receptor B) | | |
| 209933_s_at | CD300A | CD300A antigen | 0.813986 | 0.001651 |
| 216696_s_at | PRODH2 | proline dehydrogenase (oxidase) 2 | 0.813827 | 0.003259 |
| 220938_s_at | GMEB1 | glucocorticoid modulatory element binding protein 1 | 0.81236 | 0.003955 |
| 216271_x_at | SYDE1 | synapse defective 1, Rho GTPase, homolog 1 (C. elegans) | 0.812133 | 0.000592 |
| 203806_s_at | FANCA | Fanconi anemia, complementation group A /// Fanconi anemia, | 0.811827 | 0.000662 |
| | | complementation group A | | |
| 210623_at | LOC51035 | unknown protein LOC51035 | 0.811715 | 8.90E−06 |
| 208160_at | FLJ10232 | hypothetical protein FLJ10232 | 0.811444 | 0.000632 |
| 217646_at | SURF1 | surfeit 1 | 0.811438 | 0.001205 |
| 222179_at | — | — | 0.810842 | 0.000746 |
| 204484_at | PIK3C2B | phosphoinositide-3-kinase, class 2, beta polypeptide | 0.810695 | 0.00495 |
| 220698_at | MGC4294 | hypothetical protein MGC4294 | 0.810408 | 0.000573 |
| 212480_at | KIAA0376 | KIAA0376 protein | 0.810372 | 0.001581 |
| 204509_at | CA12 | carbonic anhydrase XII | 0.81026 | 0.000575 |
| 205013_s_at | ADORA2A | adenosine A2a receptor | 0.809904 | 2.19E−06 |
| 221845_s_at | CLPB | ClpB caseinolytic peptidase B homolog (E. coli) | 0.809604 | 0.003307 |
| 221293_s_at | DEF6 | differentially expressed in FDCP 6 homolog (mouse) | 0.80938 | 0.009208 |
| 203364_s_at | KIAA0652 | KIAA0652 gene product | 0.8092 | 0.001189 |
| 216502_at | ISG20L2 | interferon stimulated exonuclease gene 20 kDa-like 2 | 0.808479 | 0.000383 |
| 216811_at | — | — | 0.80722 | 0.002073 |
| 38707_r_at | E2F4 | E2F transcription factor 4, p107/p130-binding | 0.807098 | 0.003481 |
| 206791_s_at | PDE4C | phosphodiesterase 4C, cAMP-specific (phosphodiesterase E1 | 0.806942 | 0.001461 |
| | | dunce homolog, Drosophila) | | |
| 217072_at | CD300A | CD300A antigen | 0.8069 | 0.000193 |
| 213717_at | LDB3 | LIM domain binding 3 | 0.806833 | 0.003669 |
| 205685_at | CD86 | CD86 antigen (CD28 antigen ligand 2, B7-2 antigen) | 0.806044 | 0.00043 |
| 208040_s_at | MYBPC3 | myosin binding protein C, cardiac /// myosin binding protein C, | 0.805482 | 0.006134 |
| | | cardiac | | |
| 206215_at | OPCML | opioid binding protein/cell adhesion molecule-like | 0.804443 | 0.004654 |
| 200024_at | RPS5 | ribosomal protein S5 /// ribosomal protein S5 | 0.804373 | 2.71E−08 |
| 216434_at | FLJ20699 | Hypothetical protein FLJ20699 | 0.803173 | 0.008806 |
| 219887_at | FLJ10786 | hypothetical protein FLJ10786 | 0.80284 | 0.002043 |
| 215293_s_at | FRAG1 | FGF receptor activating protein 1 | 0.802814 | 0.003327 |
| 211001_at | TRIM29 | tripartite motif-containing 29 | 0.801186 | 0.00352 |
| 215592_at | — | CDNA FLJ12232 fis, clone MAMMA1001206 | 0.80007 | 0.00056 |
| 222023_at | AKAP13 | A kinase (PRKA) anchor protein 13 | 0.799597 | 1.76E−06 |
| 207601_at | SULT1B1 | sulfotransferase family, cytosolic, 1B, member 1 | 0.799559 | 0.003508 |
| 205804_s_at | TRAF3IP3 | TRAF3 interacting protein 3 | 0.799237 | 0.009694 |
| 205038_at | ZNFN1A1 | Zinc finger protein, subfamily 1A, 1 (Ikaros) | 0.798172 | 0.002108 |
| 1294_at | UBE1L | ubiquitin-activating enzyme E1-like | 0.798154 | 0.007833 |
| 209958_s_at | PTHB1 | parathyroid hormone-responsive B1 | 0.797717 | 0.001779 |
| 206361_at | GPR44 | G protein-coupled receptor 44 | 0.797095 | 0.000497 |
| 208117_s_at | LAS1L | LAS1-like (S. cerevisiae) /// LAS1-like (S. cerevisiae) | 0.796825 | 0.00129 |
| 211201_at | FSHR | follicle stimulating hormone receptor | 0.796597 | 0.000255 |
| 222331_at | — | — | 0.796315 | 0.000304 |
| 210221_at | CHRNA3 | cholinergic receptor, nicotinic, alpha polypeptide 3 | 0.796218 | 0.00672 |
| 222319_at | NAP1L4 | Nucleosome assembly protein 1-like 4 | 0.79601 | 0.004068 |
| 202002_at | ACAA2 | acetyl-Coenzyme A acyltransferase 2 (mitochondrial 3-oxoacyl- | 0.796005 | 0.001231 |
| | | Coenzyme A thiolase) | | |
| 36907_at | MVK | mevalonate kinase (mevalonic aciduria) | 0.795749 | 3.82E−05 |
| 205336_at | PVALB | parvalbumin | 0.795282 | 0.000948 |
| 216423_at | CRSP7 | Cofactor required for Sp1 transcriptional activation, subunit 7, | 0.795102 | 0.001266 |
| | | 70 kDa | | |
| 206517_at | CDH16 | cadherin 16, KSP-cadherin | 0.794476 | 0.000345 |
| 219554_at | RHCG | Rhesus blood group, C glycoprotein | 0.794444 | 5.69E−05 |
| 206991_s_at | CCR5 | chemokine (C-C motif) receptor 5 | 0.794388 | 0.001413 |
| 211365_s_at | PCDHA2 | protocadherin alpha 2 | 0.794364 | 0.001216 |
| 211261_at | — | — | 0.793915 | 0.000379 |
| 204558_at | RAD54L | RAD54-like (S. cerevisiae) | 0.793736 | 0.003131 |
| 209864_at | FRAT2 | frequently rearranged in advanced T-cell lymphomas 2 | 0.793445 | 0.007654 |
| 206886_x_at | GH1 | growth hormone 1 | 0.792857 | 0.000646 |
| 211831_s_at | THPO | thrombopoietin (myeloproliferative leukemia virus oncogene | 0.79236 | 0.009934 |
| | | ligand, megakaryocyte growth and development factor) | | |
| 203234_at | UPP1 | uridine phosphorylase 1 | 0.792279 | 0.000147 |
| 210644_s_at | LAIR1 | leukocyte-associated Ig-like receptor 1 | 0.791897 | 0.00404 |
| 214332_s_at | TSFM | Ts translation elongation factor, mitochondrial | 0.791705 | 2.98E−05 |
| 213570_at | EIF4E2 | eukaryotic translation initiation factor 4E member 2 | 0.79138 | 0.00194 |
| 217447_at | MAG | myelin associated glycoprotein | 0.79125 | 0.000143 |
| 214330_at | ATPAF2 | ATP synthase mitochondrial F1 complex assembly factor 2 | 0.790537 | 0.0039 |
| 203475_at | CYP19A1 | cytochrome P450, family 19, subfamily A, polypeptide 1 | 0.79051 | 0.00017 |
| 211403_x_at | VCX2 | variable charge, X-linked 2 | 0.790168 | 9.40E−05 |
| 207044_at | THRB | thyroid hormone receptor, beta (erythroblastic leukemia viral (v- | 0.789673 | 0.008531 |
| | | erb-a) oncogene homolog 2, avian) | | |
| 204987_at | ITIH2 | inter-alpha (globulin) inhibitor H2 | 0.789563 | 0.001902 |
| 39854_r_at | PNPLA2 | patatin-like phospholipase domain containing 2 | 0.789506 | 0.001013 |
| 210577_at | CASR | calcium-sensing receptor (hypocalciuric hypercalcemia 1, severe | 0.789336 | 0.002927 |
| | | neonatal hyperparathyroidism) | | |
| 215473_at | — | Hypothetical protein LOC284215 | 0.789187 | 0.000111 |
| 205487_s_at | VGLL1 | vestigial like 1 (Drosophila) | 0.78883 | 0.000461 |
| 214504_at | ABO | ABO blood group (transferase A, alpha 1-3-N- | 0.788684 | 0.002557 |
| | | acetylgalactosaminyltransferase; transferase B, alpha 1-3- | | |
| | | galactosyltransferase) | | |
| 216101_at | — | Full length insert cDNA clone YR67C11 | 0.787912 | 0.001857 |
| 208384_s_at | MID2 | midline 2 | 0.787341 | 0.004796 |
| 214713_at | YLPM1 | YLP motif containing 1 | 0.787238 | 0.003838 |
| 205024_s_at | RAD51 | RAD51 homolog (RecA homolog, E. coli) (S. cerevisiae) | 0.786701 | 1.72E−05 |
| 206725_x_at | BMP1 | bone morphogenetic protein 1 | 0.786448 | 0.002992 |
| 205666_at | FMO1 | flavin containing monooxygenase 1 | 0.78642 | 0.003307 |
| 219039_at | SEMA4C | sema domain, immunoglobulin domain (Ig), transmembrane | 0.785394 | 0.002913 |
| | | domain (TM) and short cytoplasmic domain, (semaphorin) 4C | | |
| 218725_at | SLC25A22 | solute carrier family 25 (mitochondrial carrier: glutamate), | 0.785388 | 0.009577 |
| | | member 22 | | |
| 205162_at | ERCC8 | excision repair cross-complementing rodent repair deficiency, | 0.785353 | 0.002648 |
| | | complementation group 8 | | |
| 216630_at | — | — | 0.785298 | 0.000289 |
| 217521_at | HAL | Histidine ammonia-lyase | 0.785231 | 0.009181 |
| 207460_at | GZMM | granzyme M (lymphocyte met-ase 1) | 0.78516 | 0.006595 |
| 220003_at | LRRC36 | leucine rich repeat containing 36 | 0.785023 | 0.00475 |
| 208844_at | VDAC3 | voltage-dependent anion channel 3 | 0.785 | 0.007067 |
| 211010_s_at | NCR3 | natural cytotoxicity triggering receptor 3 | 0.784971 | 0.008985 |
| 207861_at | CCL22 | chemokine (C-C motif) ligand 22 | 0.784835 | 0.00558 |
| 204951_at | RHOH | ras homolog gene family, member H | 0.784037 | 0.001379 |
| 219239_s_at | ZNF654 | zinc finger protein 654 | 0.783512 | 0.008124 |
| 220962_s_at | PADI1 | peptidyl arginine deiminase, type I /// peptidyl arginine | 0.783287 | 0.001691 |
| | | deiminase, type I | | |
| 216102_at | PHLDB1 | pleckstrin homology-like domain, family B, member 1 | 0.782607 | 0.004162 |
| 215167_at | CRSP2 | cofactor required for Sp1 transcriptional activation, subunit 2, | 0.78231 | 0.001092 |
| | | 150 kDa | | |
| 221173_at | USH1C | Usher syndrome 1C (autosomal recessive, severe) | 0.782221 | 0.000363 |
| 210952_at | AP4S1 | adaptor-related protein complex 4, sigma 1 subunit | 0.78188 | 2.07E−05 |
| 216512_s_at | DCT | dopachrome tautomerase (dopachrome delta-isomerase, | 0.781505 | 0.000445 |
| | | tyrosine-related protein 2) | | |
| 202877_s_at | C1QR1 | complement component 1, q subcomponent, receptor 1 /// | 0.781404 | 0.001327 |
| | | complement component 1, q subcomponent, receptor 1 | | |
| 210323_at | TEKT2 | tektin 2 (testicular) | 0.781101 | 0.005703 |
| 203996_s_at | C21orf2 | chromosome 21 open reading frame 2 | 0.780976 | 0.001676 |
| 200704_at | LITAF | lipopolysaccharide-induced TNF factor | 0.780512 | 0.006062 |
| 216018_at | RNF5 | ring finger protein 5 | 0.779803 | 0.004741 |
| 219928_s_at | CABYR | calcium binding tyrosine-(Y)-phosphorylation regulated | 0.77966 | 0.006812 |
| | | (fibrousheathin 2) | | |
| 217051_s_at | SS18 | Synovial sarcoma translocation, chromosome 18 | 0.778712 | 0.003873 |
| 207762_at | LPAL2 | lipoprotein, Lp(a)-like 2 | 0.778267 | 0.000605 |
| 213551_x_at | PCGF2 | polycomb group ring finger 2 | 0.778177 | 0.008632 |
| 204441_s_at | POLA2 | polymerase (DNA directed), alpha 2 (70 kD subunit) | 0.777742 | 0.006103 |
| 219019_at | LRDD | leucine-rich repeats and death domain containing | 0.777704 | 0.004462 |
| 221385_s_at | GPR41 /// GPR42 | G protein-coupled receptor 41 /// G protein-coupled receptor 42 | 0.777162 | 0.001749 |
| 206916_x_at | TAT | tyrosine aminotransferase | 0.77691 | 7.20E−05 |
| 205297_s_at | CD79B | CD79B antigen (immunoglobulin-associated beta) | 0.775791 | 0.001851 |
| 211188_at | CD84 | CD84 antigen (leukocyte antigen) | 0.775755 | 0.000579 |
| 220135_s_at | SLC7A9 | solute carrier family 7 (cationic amino acid transporter, y+ | 0.775623 | 0.001369 |
| | | system), member 9 | | |
| 216096_s_at | NRXN1 | neurexin 1 | 0.775147 | 5.45E−05 |
| 217096_at | PCLO | piccolo (presynaptic cytomatrix protein) | 0.774706 | 0.008978 |
| 220152_at | C10orf95 | chromosome 10 open reading frame 95 | 0.774477 | 0.000671 |
| 215247_at | LOC339692 | hypothetical protein LOC339692 | 0.774086 | 0.005562 |
| 206154_at | RLBP1 | retinaldehyde binding protein 1 | 0.773343 | 0.003012 |
| 213432_at | MUC5B | mucin 5, subtype B, tracheobronchial | 0.77328 | 3.33E−05 |
| 208535_x_at | COL13A1 | collagen, type XIII, alpha 1 | 0.772756 | 0.000316 |
| 206480_at | LTC4S | leukotriene C4 synthase | 0.772733 | 0.000977 |
| 218960_at | TMPRSS4 | transmembrane protease, serine 4 | 0.772683 | 0.008895 |
| 222169_x_at | SH2D3A | SH2 domain containing 3A | 0.772376 | 0.005787 |
| 204789_at | FMNL1 | formin-like 1 | 0.771858 | 0.002657 |
| 220965_s_at | RSHL1 | radial spokehead-like 1 /// radial spokehead-like 1 | 0.771549 | 0.000651 |
| 214947_at | FLJ11127 | Hypothetical protein FLJ11127 | 0.771387 | 0.001583 |
| 201759_at | TBCD | tubulin-specific chaperone d | 0.770595 | 0.007336 |
| 206156_at | GJB5 | gap junction protein, beta 5 (connexin 31.1) | 0.770371 | 0.001556 |
| 206672_at | AQP2 | aquaporin 2 (collecting duct) | 0.770297 | 0.006014 |
| 215040_at | FLJ11712 | Hypothetical protein FLJ11712 | 0.770163 | 0.001169 |
| 211053_at | KCNG1 | potassium voltage-gated channel, subfamily G, member 1 /// | 0.770058 | 0.003538 |
| | | potassium voltage-gated channel, subfamily G, member 1 | | |
| 208278_s_at | — | — | 0.769908 | 0.000496 |
| 217374_x_at | TRGV5 | T cell receptor gamma variable 5 | 0.769771 | 0.003118 |
| 213870_at | COL11A2 | collagen, type XI, alpha 2 | 0.768957 | 0.004164 |
| 216240_at | — | — | 0.768727 | 2.27E−05 |
| 217969_at | C11orf2 | chromosome 11 open reading frame2 | 0.768471 | 0.00017 |
| 221108_at | LOC51233 | hypothetical protein LOC51233 | 0.767947 | 0.000851 |
| 220423_at | PLA2G2D | phospholipase A2, group IID | 0.767944 | 0.005162 |
| 200019_s_at | FAU | Finkel-Biskis-Reilly murine sarcoma virus (FBR-MuSV) | 0.767922 | 1.31E−05 |
| | | ubiquitously expressed (fox derived); ribosomal protein S30 /// | | |
| | | Finkel-Biskis-Reilly murine sarcoma virus (FBR-MuSV) | | |
| | | ubiquitously expressed (fox derived); ribosomal protein S30 | | |
| 211153_s_at | TNFSF11 | tumor necrosis factor (ligand) superfamily, member 11 | 0.767237 | 0.003445 |
| 218429_s_at | FLJ11286 | hypothetical protein FLJ11286 | 0.767151 | 0.003267 |
| 220181_x_at | SLC30A5 | solute carrier family 30 (zinc transporter), member 5 | 0.766698 | 0.005938 |
| 214576_at | KRTHA6 | keratin, hair, acidic, 6 | 0.766311 | 0.000486 |
| 207715_at | CRYGB | crystallin, gamma B | 0.765847 | 7.38E−07 |
| 211332_x_at | HFE | hemochromatosis | 0.765304 | 0.004529 |
| 219748_at | TREML2 | triggering receptor expressed on myeloid cells-like 2 | 0.765109 | 0.000312 |
| 220017_x_at | CYP2C9 | cytochrome P450, family 2, subfamily C, polypeptide 9 | 0.76507 | 0.006102 |
| 214626_s_at | GANAB | glucosidase, alpha; neutral AB | 0.764832 | 0.004482 |
| 220466_at | CCDC15 | coiled-coil domain containing 15 | 0.764772 | 0.008919 |
| 217233_at | — | — | 0.764632 | 0.001359 |
| 220533_at | — | — | 0.764354 | 0.003745 |
| 221371_at | TNFSF18 | tumor necrosis factor (ligand) superfamily, member 18 | 0.764067 | 0.000419 |
| 207923_x_at | PAX8 | paired box gene 8 | 0.763831 | 0.003757 |
| 206433_s_at | SPOCK3 | sparc/osteonectin, cwcv and kazal-like domains proteoglycan | 0.76366 | 0.004355 |
| | | (testican) 3 | | |
| 216815_at | — | — | 0.763609 | 0.000491 |
| 210938_at | IPF1 | insulin promoter factor 1, homeodomain transcription factor | 0.763097 | 0.000252 |
| 632_at | GSK3A | glycogen synthase kinase 3 alpha | 0.763058 | 0.005278 |
| 217031_at | KRTHB4 | keratin, hair, basic, 4 | 0.763017 | 2.05E−05 |
| 220449_at | MGC5566 | Hypothetical protein MGC5566 | 0.761084 | 0.00779 |
| 205071_x_at | XRCC4 | X-ray repair complementing defective repair in Chinese hamster | 0.76088 | 0.004838 |
| | | cells 4 | | |
| 208959_s_at | TXNDC4 | thioredoxin domain containing 4 (endoplasmic reticulum) | 0.760576 | 0.00595 |
| 215788_at | LOC339457 | hypothetical protein LOC339457 | 0.760313 | 0.000587 |
| 213783_at | MFNG | Manic fringe homolog (Drosophila) | 0.760231 | 0.002112 |
| 203069_at | SV2A | synaptic vesicle glycoprotein 2A | 0.760159 | 0.002689 |
| 212750_at | PPP1R16B | protein phosphatase 1, regulatory (inhibitor) subunit 16B | 0.760146 | 0.001868 |
| 207739_s_at | GAGE1 /// | G antigen 1 /// G antigen 2 /// G antigen 3 /// G antigen 4 /// G | 0.75952 | 9.99E−05 |
| | GAGE2 /// | antigen 5 /// G antigen 6 /// G antigen 7 /// G antigen 7B /// G | | |
| | GAGE3 /// | antigen 8 | | |
| | GAGE4 /// | | | |
| | GAGE5 /// | | | |
| | GAGE6 /// | | | |
| | GAGE7 /// | | | |
| | GAGE7B /// | | | |
| | GAGE8 | | | |
| 206435_at | GALGT | UDP-N-acetyl-alpha-D-galactosamine:(N-acetylneuraminyl)- | 0.759372 | 0.003169 |
| | | galactosylglucosylceramide N-acetylgalactosaminyltransferase | | |
| | | (GalNAc-T) | | |
| 205900_at | KRT1 | keratin 1 (epidermolytic hyperkeratosis) | 0.759183 | 0.003742 |
| 205148_s_at | CLCN4 | chloride channel 4 | 0.758576 | 0.005136 |
| 209791_at | PADI2 | peptidyl arginine deiminase, type II | 0.758121 | 0.006275 |
| 220042_x_at | HIVEP3 | human immunodeficiency virus type I enhancer binding protein 3 | 0.757471 | 0.00162 |
| 219755_at | CBX8 | chromobox homolog 8 (Pc class homolog, Drosophila) | 0.757315 | 0.005612 |
| 210921_at | — | — | 0.757085 | 0.001124 |
| 205783_at | KLK13 | kallikrein 13 | 0.756988 | 0.008731 |
| 217590_s_at | TRPA1 | transient receptor potential cation channel, subfamily A, member 1 | 0.756755 | 0.000977 |
| 206573_at | KCNQ3 | potassium voltage-gated channel, KQT-like subfamily, member 3 | 0.756527 | 0.009349 |
| 218153_at | FLJ12118 | hypothetical protein FLJ12118 | 0.756084 | 0.008173 |
| 203768_s_at | STS | steroid sulfatase (microsomal), arylsulfatase C, isozyme S | 0.755637 | 0.001909 |
| 221373_x_at | PSPN | persephin | 0.755625 | 2.28E−05 |
| 211825_s_at | FLI1 | Friend leukemia virus integration 1 | 0.755472 | 0.001633 |
| 201182_s_at | CHD4 | chromodomain helicase DNA binding protein 4 | 0.755267 | 0.001491 |
| 203257_s_at | MGC4707 | MGC4707 protein | 0.754948 | 0.001332 |
| 213834_at | IQSEC3 | IQ motif and Sec7 domain 3 | 0.75487 | 0.001324 |
| 214080_x_at | PRKCSH | protein kinase C substrate 80K-H | 0.754731 | 0.000808 |
| 219879_s_at | MGC3130 | hypothetical protein MGC3130 | 0.753807 | 0.003639 |
| 215154_at | ULK2 | Unc-51-like kinase 2 (C. elegans) | 0.753204 | 0.004838 |
| 219072_at | BCL7C | B-cell CLL/lymphoma 7C | 0.753019 | 0.006191 |
| 214846_s_at | ALPK3 | alpha-kinase 3 | 0.752754 | 0.005488 |
| 205130_at | RAGE | renal tumor antigen | 0.750887 | 0.008519 |
| 58367_s_at | ZNF419 | zinc finger protein 419 | 0.750783 | 0.000734 |
| 208352_x_at | ANK1 | ankyrin 1, erythrocytic | 0.750502 | 0.005223 |
| 206778_at | CRYBB2 | crystallin, beta B2 | 0.750164 | 0.002763 |
| 206927_s_at | GUCY1A2 | guanylate cyclase 1, soluble, alpha 2 | 0.750102 | 0.000677 |
| 211049_at | TLX2 | T-cell leukemia, homeobox 2 /// T-cell leukemia, homeobox 2 | 0.748417 | 0.003895 |
| 207354_at | CCL16 | chemokine (C-C motif) ligand 16 | 0.748113 | 1.99E−05 |
| 48580_at | CXXC1 | CXXC finger 1 (PHD domain) | 0.747662 | 0.003842 |
| 207509_s_at | LAIR2 | leukocyte-associated Ig-like receptor 2 | 0.746935 | 0.001224 |
| 208277_at | PITX3 | paired-like homeodomain transcription factor 3 | 0.746771 | 0.006171 |
| 209976_s_at | CYP2E1 | cytochrome P450, family 2, subfamily E, polypeptide 1 | 0.746595 | 0.002227 |
| 219309_at | FLJ23584 | hypothetical protein FLJ23584 | 0.745824 | 0.000907 |
| 220139_at | DNMT3L | DNA (cytosine-5-)-methyltransferase 3-like | 0.745726 | 0.000366 |
| 210747_at | HLA-DQB1 | major histocompatibility complex, class II, DQ beta 1 | 0.74506 | 0.000739 |
| 216019_x_at | PHLDB1 | pleckstrin homology-like domain, family B, member 1 | 0.74487 | 0.006501 |
| 210654_at | TNFRSF10D | tumor necrosis factor receptor superfamily, member 10d, decoy | 0.744763 | 0.001281 |
| | | with truncated death domain | | |
| 213448_at | GBA | Glucosidase, beta; acid (includes glucosylceramidase) | 0.742498 | 0.001709 |
| 205710_at | LRP2 | low density lipoprotein-related protein 2 | 0.741673 | 0.00125 |
| 216991_at | ZNF224 | zinc finger protein 224 | 0.741594 | 0.000518 |
| 216611_s_at | SLC6A2 | solute carrier family 6 (neurotransmitter transporter, | 0.741249 | 0.00774 |
| | | noradrenalin), member 2 | | |
| 222100_at | CYP2E1 | Cytochrome P450, family 2, subfamily E, polypeptide 1 | 0.74094 | 0.000145 |
| 221914_at | SYN1 | synapsin I | 0.73969 | 0.004662 |
| 210451_at | PKLR | pyruvate kinase, liver and RBC | 0.739627 | 0.001054 |
| 216275_at | BUB1 | BUB1 budding uninhibited by benzimidazoles 1 homolog (yeast) | 0.739568 | 0.002039 |
| 206747_at | KIAA0514 | KIAA0514 | 0.739474 | 0.005984 |
| 211442_x_at | CYP3A43 | cytochrome P450, family 3, subfamily A, polypeptide 43 | 0.739357 | 0.000429 |
| 213751_at | LOC284352 | hypothetical protein LOC284352 | 0.739197 | 0.004583 |
| 214178_s_at | SOX2 | SRY (sex determining region Y)-box 2 | 0.739108 | 0.001426 |
| 222300_at | CAPZA1 | Capping protein (actin filament) muscle Z-line, alpha 1 | 0.739085 | 0.005602 |
| 216141_at | — | — | 0.738963 | 0.004804 |
| 219632_s_at | TRPV1 | transient receptor potential cation channel, subfamily V, member 1 | 0.738926 | 0.004412 |
| 222374_at | BTRC | beta-transducin repeat containing | 0.738846 | 0.003263 |
| 221969_at | PAX5 | Paired box gene 5 (B-cell lineage specific activator) | 0.73839 | 5.62E−05 |
| 219704_at | YBX2 | Y box binding protein 2 | 0.738303 | 0.000803 |
| 217225_x_at | NOMO2 | NODAL modulator 2 | 0.737947 | 0.005815 |
| 208564_at | KCNA2 | potassium voltage-gated channel, shaker-related subfamily, | 0.737875 | 0.000582 |
| | | member 2 | | |
| 207404_s_at | HTR1E | 5-hydroxytryptamine (serotonin) receptor 1E | 0.737621 | 0.002553 |
| 221439_at | RBBP9 | retinoblastoma binding protein 9 | 0.736647 | 3.29E−05 |
| 213550_s_at | PRO1580 | hypothetical protein PRO1580 | 0.736426 | 0.005736 |
| 205646_s_at | PAX6 | paired box gene 6 (aniridia, keratitis) | 0.73633 | 0.003352 |
| 220384_at | TXNDC3 | thioredoxin domain containing 3 (spermatozoa) | 0.735947 | 0.001248 |
| 219331_s_at | FLJ10748 | hypothetical protein FLJ10748 | 0.735735 | 0.001527 |
| 207716_at | KRTHA8 | keratin, hair, acidic, 8 | 0.735276 | 0.000705 |
| 220763_at | HSPA12A | heat shock 70 kDa protein 12A | 0.734892 | 0.005677 |
| 220733_at | SLC26A1 | solute carrier family 26 (sulfate transporter), member 1 | 0.734381 | 0.007889 |
| 216157_at | — | CDNA FLJ14169 fis, clone NT2RP2002056 | 0.734356 | 0.007663 |
| 214025_at | DDX28 | DEAD (Asp-Glu-Ala-Asp) box polypeptide 28 | 0.73419 | 0.001568 |
| 205812_s_at | TMED9 | transmembrane emp24 protein transport domain containing 9 | 0.734073 | 0.001812 |
| 218365_s_at | DARS2 | aspartyl-tRNA synthetase 2 (mitochondrial) | 0.733985 | 0.001568 |
| 208434_at | MDS1 | myelodysplasia syndrome 1 | 0.733976 | 0.000405 |
| 220045_at | NEUROD6 | neurogenic differentiation 6 | 0.733942 | 0.002713 |
| 217172_at | — | — | 0.73366 | 3.75E−05 |
| 216800_at | — | CDNA: FLJ23416 fis, clone HEP20790 | 0.733561 | 0.000902 |
| 209840_s_at | LRRN3 | leucine rich repeat neuronal 3 | 0.733237 | 4.43E−05 |
| 215644_at | ZNF518 | zinc finger protein 518 | 0.732356 | 0.000236 |
| 217928_s_at | SAPS3 | SAPS domain family, member 3 | 0.732275 | 0.000443 |
| 216968_at | MASP2 | mannan-binding lectin serine peptidase 2 | 0.732254 | 0.008193 |
| 207841_at | SPIN2 | spindlin family, member 2 | 0.731694 | 0.001292 |
| 204561_x_at | APOC2 | apolipoprotein C-II | 0.730618 | 0.005238 |
| 207396_s_at | ALG3 | asparagine-linked glycosylation 3 homolog (yeast, alpha-1,3- | 0.729692 | 0.004543 |
| | | mannosyltransferase) | | |
| 208230_s_at | NRG1 | neuregulin 1 | 0.728905 | 0.005491 |
| 220146_at | TLR7 | toll-like receptor 7 | 0.728674 | 0.00041 |
| 220033_at | — | — | 0.728663 | 0.002745 |
| 217611_at | ERICH1 | glutamate-rich 1 | 0.72831 | 0.002727 |
| 214380_at | PRPF31 | PRP31 pre-mRNA processing factor 31 homolog (yeast) | 0.727856 | 0.006891 |
| 214407_x_at | GYPB | glycophorin B (includes Ss blood group) | 0.727724 | 0.000463 |
| 206076_at | B7 | B7 gene | 0.727497 | 0.007895 |
| 215639_at | SH2D3C | SH2 domain containing 3C | 0.72684 | 0.000244 |
| 221684_s_at | NYX | nyctalopin | 0.72661 | 0.006153 |
| 206126_at | BLR1 | Burkitt lymphoma receptor 1, GTP binding protein (chemokine | 0.726313 | 0.007684 |
| | | (C—X—C motif) receptor 5) | | |
| 215005_at | EFCBP2 | EF-hand calcium binding protein 2 | 0.725687 | 0.003372 |
| 212850_s_at | LRP4 | low density lipoprotein receptor-related protein 4 | 0.725597 | 0.002745 |
| 213450_s_at | ICOSLG | inducible T-cell co-stimulator ligand | 0.724751 | 0.008206 |
| 201995_at | EXT1 | exostoses (multiple) 1 | 0.724582 | 0.004546 |
| 48659_at | IIP45 | invasion inhibitory protein 45 | 0.724086 | 0.007762 |
| 222327_x_at | OR7E156P | olfactory receptor, family 7, subfamily E, member 156 | 0.723604 | 0.005347 |
| | | pseudogene | | |
| 218070_s_at | GMPPA | GDP-mannose pyrophosphorylase A | 0.722533 | 0.007225 |
| 214639_s_at | HOXA1 | homeo box A1 | 0.722029 | 6.38E−06 |
| 203268_s_at | DRG2 | developmentally regulated GTP binding protein 2 | 0.721389 | 5.99E−05 |
| 211133_x_at | LILRB2 /// | leukocyte immunoglobulin-like receptor, subfamily B (with TM | 0.721255 | 0.002849 |
| | LILRB3 | and ITIM domains), member 2 /// leukocyte immunoglobulin-like | | |
| | | receptor, subfamily B (with TM and ITIM domains), member 3 | | |
| 216156_at | RECK | reversion-inducing-cysteine-rich protein with kazal motifs | 0.720137 | 1.80E−07 |
| 206489_s_at | DLGAP1 | discs, large (Drosophila) homolog-associated protein 1 | 0.71899 | 0.007626 |
| 221164_x_at | CHST5 | carbohydrate (N-acetylglucosamine 6-O) sulfotransferase 5 | 0.718825 | 0.001232 |
| 211154_at | THPO | thrombopoietin (myeloproliferative leukemia virus oncogene | 0.717864 | 0.000856 |
| | | ligand, megakaryocyte growth and development factor) | | |
| 216794_at | — | CDNA clone IMAGE: 30320498 | 0.7177 | 0.003651 |
| 211565_at | SH3GL3 | SH3-domain GRB2-like 3 | 0.717444 | 0.00335 |
| 202415_s_at | HSPBP1 | hsp70-interacting protein | 0.717136 | 0.000913 |
| 215374_at | PAPOLA | Poly(A) polymerase alpha | 0.716785 | 0.001155 |
| 208253_at | SIGLEC8 | sialic acid binding Ig-like lectin 8 | 0.716701 | 5.56E−05 |
| 212346_s_at | MXD4 | MAX dimerization protein 4 | 0.716128 | 0.006525 |
| 217295_at | MUC8 | mucin 8, tracheobronchial | 0.716037 | 0.006062 |
| 31861_at | IGHMBP2 | immunoglobulin mu binding protein 2 | 0.716002 | 0.001943 |
| 221077_at | ARMC4 | armadillo repeat containing 4 | 0.715692 | 0.001454 |
| 221084_at | HTR3B | 5-hydroxytryptamine (serotonin) receptor 3B | 0.715425 | 0.003807 |
| 207698_at | C6orf123 | chromosome 6 open reading frame 123 | 0.71537 | 0.003091 |
| 205177_at | TNNI1 | troponin I type 1 (skeletal, slow) | 0.714778 | 0.009876 |
| 206166_s_at | CLCA2 | chloride channel, calcium activated, family member 2 | 0.714564 | 0.005892 |
| 1494_f_at | CYP2A6 | cytochrome P450, family 2, subfamily A, polypeptide 6 | 0.714226 | 0.006749 |
| 214539_at | SERPINB10 | serpin peptidase inhibitor, clade B (ovalbumin), member 10 | 0.714086 | 0.008971 |
| 202339_at | SYMPK | symplekin | 0.713519 | 0.000504 |
| 221131_at | A4GNT | alpha-1,4-N-acetylglucosaminyltransferase | 0.713416 | 0.000258 |
| 216561_x_at | — | — | 0.713341 | 0.002312 |
| 211524_at | NFKB2 | nuclear factor of kappa light polypeptide gene enhancer in B- | 0.71322 | 2.28E−05 |
| | | cells 2 (p49/p100) | | |
| 217760_at | TRIM44 | tripartite motif-containing 44 | 0.713136 | 0.005436 |
| 220120_s_at | EPB41L4A | erythrocyte membrane protein band 4.1 like 4A | 0.713067 | 0.000411 |
| 208922_s_at | NXF1 | nuclear RNA export factor 1 | 0.711864 | 0.008214 |
| 216432_at | SLC28A2 | Solute carrier family 28 (sodium-coupled nucleoside transporter), | 0.71158 | 0.006507 |
| | | member 2 | | |
| 210422_x_at | SLC11A1 | solute carrier family 11 (proton-coupled divalent metal ion | 0.711371 | 0.004348 |
| | | transporters), member 1 | | |
| 204639_at | ADA | adenosine deaminase | 0.710666 | 0.000152 |
| 208465_at | GRM2 | glutamate receptor, metabotropic 2 | 0.710545 | 0.00449 |
| 210343_s_at | SLC22A6 | solute carrier family 22 (organic anion transporter), member 6 | 0.710136 | 0.009276 |
| 220421_at | BTNL8 | butyrophilin-like 8 | 0.709998 | 0.001877 |
| 208516_at | MTNR1B | melatonin receptor 1B | 0.7089 | 0.001154 |
| 221102_s_at | TRPM6 | transient receptor potential cation channel, subfamily M, member 6 | 0.708872 | 0.004025 |
| 211897_s_at | CRHR1 | corticotropin releasing hormone receptor 1 | 0.708683 | 0.008851 |
| 208279_s_at | CDRT1 | CMT1A duplicated region transcript 1 | 0.708091 | 0.000593 |
| 214851_at | HNF4A | hepatocyte nuclear factor 4, alpha | 0.707757 | 0.001902 |
| 211545_at | GHRHR | growth hormone releasing hormone receptor | 0.707102 | 0.000342 |
| 205629_s_at | CRH | corticotropin releasing hormone | 0.706933 | 0.003064 |
| 213565_s_at | SMAD6 | SMAD, mothers against DPP homolog 6 (Drosophila) | 0.706596 | 0.007998 |
| 206561_s_at | AKR1B10 | aldo-keto reductase family 1, member B10 (aldose reductase) | 0.706432 | 0.000769 |
| 216390_at | LCAT | Lecithin-cholesterol acyltransferase | 0.705841 | 0.001722 |
| 218805_at | GIMAP5 | GTPase, IMAP family member 5 /// GTPase, IMAP family | 0.705832 | 0.007558 |
| | | member 5 | | |
| 204579_at | FGFR4 | fibroblast growth factor receptor 4 | 0.705574 | 0.00644 |
| 219674_s_at | PRO2900 | hypothetical protein PRO2900 | 0.705443 | 0.000727 |
| 216677_at | ZNF154 | zinc finger protein 154 (pHZ-92) | 0.70535 | 0.001091 |
| 204556_s_at | DZIP1 | DAZ interacting protein 1 | 0.705181 | 0.005691 |
| 205339_at | SIL | TAL1 (SCL) interrupting locus | 0.704952 | 0.005112 |
| 211241_at | ANXA2P3 | annexin A2 pseudogene 3 | 0.70495 | 0.004679 |
| 221287_at | RNASEL | ribonuclease L (2′,5′-oligoisoadenylate synthetase-dependent) | 0.7045 | 0.000899 |
| 215097_at | CAPZB | capping protein (actin filament) muscle Z-line, beta | 0.704142 | 0.00133 |
| 220505_at | C9orf53 | chromosome 9 open reading frame 53 | 0.703581 | 0.003953 |
| 210955_at | CASP10 | caspase 10, apoptosis-related cysteine peptidase | 0.703033 | 0.004801 |
| 210118_s_at | IL1A | interleukin 1, alpha | 0.7026 | 1.00E−06 |
| 216128_at | TBCD | tubulin-specific chaperone d | 0.701967 | 0.003251 |
| 211259_s_at | BMP7 | bone morphogenetic protein 7 (osteogenic protein 1) | 0.701912 | 0.007575 |
| 211258_s_at | TGFA | transforming growth factor, alpha | 0.701745 | 0.002606 |
| 220388_at | FER1L4 | fer-1-like 4 (C. elegans) | 0.701102 | 0.006934 |
| 203472_s_at | SLCO2B1 | solute carrier organic anion transporter family, member 2B1 | 0.701062 | 0.004284 |
| 207235_s_at | GRM5 | glutamate receptor, metabotropic 5 | 0.70104 | 5.94E−05 |
| 201482_at | QSCN6 | quiescin Q6 | 0.701013 | 0.008192 |
| 32699_s_at | PVR | poliovirus receptor | 0.700801 | 2.20E−05 |
| 214228_x_at | TNFRSF4 | tumor necrosis factor receptor superfamily, member 4 | 0.700764 | 0.000871 |
| 216290_x_at | — | CDNA FLJ10002 fis, clone HEMBA1000046 | 0.700045 | 0.000296 |
| 217508_s_at | C18orf25 | chromosome 18 open reading frame 25 | 0.699784 | 0.002469 |
| 206000_at | MEP1A | meprin A, alpha (PABA peptide hydrolase) | 0.699657 | 0.00511 |
| 207073_at | CDKL2 | cyclin-dependent kinase-like 2 (CDC2-related kinase) | 0.699645 | 0.001776 |
| 208630_at | HADHA | hydroxyacyl-Coenzyme A dehydrogenase/3-ketoacyl-Coenzyme | 0.698483 | 5.30E−05 |
| | | A thiolase/enoyl-Coenzyme A hydratase (trifunctional protein), | | |
| | | alpha subunit | | |
| 214099_s_at | PDE4DIP | phosphodiesterase 4D interacting protein (myomegalin) | 0.698245 | 0.007284 |
| 210274_at | MAGEA8 | melanoma antigen family A, 8 | 0.697886 | 0.000904 |
| 210957_s_at | AFF2 | AF4/FMR2 family, member 2 | 0.696872 | 0.000701 |
| 207527_at | KCNJ9 | potassium inwardly-rectifying channel, subfamily J, member 9 | 0.696617 | 0.006359 |
| 211855_s_at | SLC25A14 | solute carrier family 25 (mitochondrial carrier, brain), member 14 | 0.696571 | 0.003166 |
| 220508_at | CESK1 | T-complex protein 1 | 0.695884 | 0.002589 |
| 205491_s_at | GJB3 | gap junction protein, beta 3, 31 kDa (connexin 31) | 0.695622 | 0.007144 |
| 58900_at | LOC222070 | hypothetical protein LOC222070 | 0.695468 | 0.003959 |
| 221388_at | OR1A1 | olfactory receptor, family 1, subfamily A, member 1 | 0.695239 | 0.00079 |
| 216208_s_at | CREBL1 | cAMP responsive element binding protein-like 1 | 0.693932 | 0.000465 |
| 216545_at | LOC441886 | similar to Aspartate aminotransferase, mitochondrial precursor | 0.693901 | 5.69E−06 |
| | | (Transaminase A) (Glutamate oxaloacetate transaminase-2) | | |
| 210672_s_at | C16orf35 | chromosome 16 open reading frame 35 | 0.69376 | 0.006977 |
| 206671_at | SAG | S-antigen; retina and pineal gland (arrestin) | 0.693712 | 0.000763 |
| 216959_x_at | NRCAM | neuronal cell adhesion molecule | 0.693669 | 0.003507 |
| 211039_at | CHRNA1 | cholinergic receptor, nicotinic, alpha polypeptide 1 (muscle) /// | 0.693657 | 0.00497 |
| | | cholinergic receptor, nicotinic, alpha polypeptide 1 (muscle) | | |
| 207897_at | CRHR2 | corticotropin releasing hormone receptor 2 | 0.693251 | 0.001701 |
| 205149_s_at | CLCN4 | chloride channel 4 | 0.692806 | 0.001034 |
| 220871_at | — | — | 0.692677 | 0.000183 |
| 205372_at | PLAG1 | pleiomorphic adenoma gene 1 | 0.692605 | 0.006095 |
| 206774_at | FRMPD1 | FERM and PDZ domain containing 1 | 0.691499 | 0.001573 |
| 215784_at | CD1E | CD1E antigen, e polypeptide | 0.691485 | 0.001671 |
| 221469_at | GPR32 | G protein-coupled receptor 32 | 0.691044 | 0.000573 |
| 210923_at | SLC1A7 | solute carrier family 1 (glutamate transporter), member 7 | 0.69054 | 0.006606 |
| 206939_at | DCC | deleted in colorectal carcinoma | 0.689678 | 0.002502 |
| 217180_at | — | (clone 9F2L) Ig rearranged L-chain mRNA V-region, 5′ end | 0.689054 | 0.008912 |
| 207772_s_at | HRMT1L4 | HMT1 hnRNP methyltransferase-like 4 (S. cerevisiae) | 0.68874 | 0.000267 |
| 207427_at | ACR | acrosin | 0.688695 | 0.006429 |
| 206867_at | GCKR | glucokinase (hexokinase 4) regulator | 0.688571 | 0.005492 |
| 210313_at | LILRA4 | leukocyte immunoglobulin-like receptor, subfamily A (with TM | 0.688113 | 0.007393 |
| | | domain), member 4 | | |
| 201271_s_at | RALY | RNA binding protein, autoantigenic (hnRNP-associated with | 0.687496 | 0.00124 |
| | | lethal yellow homolog (mouse)) | | |
| 203422_at | POLD1 | polymerase (DNA directed), delta 1, catalytic subunit 125 kDa | 0.687435 | 0.004104 |
| 221175_at | FLJ22173 | hypothetical protein LOC80111 | 0.687281 | 4.03E−05 |
| 207184_at | SLC6A13 | solute carrier family 6 (neurotransmitter transporter, GABA), | 0.686468 | 0.00648 |
| | | member 13 | | |
| 207528_s_at | SLC7A11 | solute carrier family 7, (cationic amino acid transporter, y+ | 0.686428 | 0.007897 |
| | | system) member 11 | | |
| 214901_at | ZNF8 | zinc finger protein 8 (clone HF.18) | 0.686388 | 0.009915 |
| 220635_at | PSORS1C2 | psoriasis susceptibility 1 candidate 2 | 0.686387 | 0.008048 |
| 215480_at | KIAA0509 | KIAA0509 protein | 0.686352 | 0.000874 |
| 207084_at | POU3F2 | POU domain, class 3, transcription factor 2 | 0.685917 | 0.00014 |
| 219224_x_at | ZNF408 | zinc finger protein 408 | 0.6858 | 0.004119 |
| 205125_at | PLCD1 | phospholipase C, delta 1 | 0.685605 | 0.009962 |
| 208007_at | — | — | 0.685302 | 0.004525 |
| 215449_at | BZRPL1 | benzodiazapine receptor (peripheral)-like 1 | 0.685249 | 0.006847 |
| 222098_s_at | ZNF42 | Zinc finger protein 42 (myeloid-specific retinoic acid-responsive) | 0.685202 | 2.19E−05 |
| 207214_at | SPINK4 | serine peptidase inhibitor, Kazal type 4 | 0.684494 | 0.001924 |
| 214480_at | ETV3 | ets variant gene 3 | 0.68431 | 0.000781 |
| 214847_s_at | GPSM3 | G-protein signalling modulator 3 (AGS3-like, C. elegans) | 0.68402 | 0.000718 |
| 221128_at | ADAM19 | ADAM metallopeptidase domain 19 (meltrin beta) | 0.683503 | 0.004478 |
| 220507_s_at | UPB1 | ureidopropionase, beta | 0.682742 | 0.009328 |
| 207663_x_at | GAGE3 | G antigen 3 | 0.68228 | 0.003566 |
| 216946_at | HLA-DOA | major histocompatibility complex, class II, DO alpha | 0.68216 | 0.000664 |
| 214780_s_at | MYO9B | myosin IXB | 0.682094 | 0.005034 |
| 207368_at | HTR1D | 5-hydroxytryptamine (serotonin) receptor 1D | 0.68205 | 0.002086 |
| 214342_at | ATXN7L1 /// | ataxin 7-like 1 /// ataxin 7-like 4 | 0.681833 | 0.000681 |
| | ATXN7L4 | | | |
| 218406_x_at | NENF | neuron derived neurotrophic factor | 0.681533 | 0.009775 |
| 217458_at | — | — | 0.681019 | 0.004983 |
| 215845_x_at | — | CDNA: FLJ22840 fis, clone KAIA4709 | 0.680687 | 0.004799 |
| 207773_x_at | CYP3A43 | cytochrome P450, family 3, subfamily A, polypeptide 43 | 0.679836 | 0.004517 |
| 215507_x_at | — | — | 0.678624 | 9.67E−07 |
| 206979_at | C8B | complement component 8, beta polypeptide | 0.678523 | 0.001294 |
| 214950_at | IL9R /// | interleukin 9 receptor /// similar to interleukin 9 receptor | 0.678421 | 0.005302 |
| | LOC400481 | | | |
| 48612_at | N4BP1 | Nedd4 binding protein 1 | 0.677876 | 0.006656 |
| 216015_s_at | CIAS1 | cold autoinflammatory syndrome 1 | 0.677614 | 0.003641 |
| 215340_at | ADCY1 | adenylate cyclase 1 (brain) | 0.677261 | 0.008956 |
| 215129_at | PIK3C2G | phosphoinositide-3-kinase, class 2, gamma polypeptide | 0.676962 | 0.001151 |
| 206706_at | NTF3 | neurotrophin 3 | 0.676784 | 0.001065 |
| 205742_at | TNNI3 | troponin I type 3 (cardiac) | 0.676275 | 0.000732 |
| 211030_s_at | SLC6A6 | solute carrier family 6 (neurotransmitter transporter, taurine), | 0.675893 | 0.004243 |
| | | member 6 /// solute carrier family 6 (neurotransmitter transporter, | | |
| | | taurine), member 6 | | |
| 208458_at | SCNN1D | sodium channel, nonvoltage-gated 1, delta | 0.67583 | 0.006252 |
| 207091_at | P2RX7 | purinergic receptor P2X, ligand-gated ion channel, 7 | 0.675672 | 0.004169 |
| 220813_at | CYSLTR2 | cysteinyl leukotriene receptor 2 | 0.675225 | 0.000809 |
| 208234_x_at | FGFR2 | fibroblast growth factor receptor 2 (bacteria-expressed kinase, | 0.675055 | 0.002147 |
| | | keratinocyte growth factor receptor, craniofacial dysostosis 1, | | |
| | | Crouzon syndrome, Pfeiffer syndrome, Jackson-Weiss | | |
| | | syndrome) | | |
| 208006_at | FOXI1 | forkhead box I1 | 0.674062 | 0.006671 |
| 208409_at | SLC14A2 | solute carrier family 14 (urea transporter), member 2 | 0.673791 | 0.00052 |
| 216582_at | LOC441135 | similar to b24o18.3 (POM121 membrane glycoprotein (rat | 0.673689 | 0.003291 |
| | | homolog)-like 2) | | |
| 210411_s_at | GRIN2B | glutamate receptor, ionotropic, N-methyl D-aspartate 2B | 0.673602 | 0.001328 |
| 207310_s_at | NOS1 | nitric oxide synthase 1 (neuronal) | 0.673555 | 6.66E−05 |
| 218399_s_at | CDCA4 | cell division cycle associated 4 | 0.673371 | 0.002387 |
| 216519_s_at | PROSC | Proline synthetase co-transcribed homolog (bacterial) | 0.673158 | 9.50E−05 |
| 216599_x_at | SLC22A6 | solute carrier family 22 (organic anion transporter), member 6 | 0.672989 | 0.009312 |
| 210885_s_at | TRIM15 | tripartite motif-containing 15 | 0.672987 | 0.005886 |
| 216866_s_at | COL14A1 | collagen, type XIV, alpha 1 (undulin) | 0.672729 | 0.002016 |
| 216201_at | — | CDNA: FLJ21586 fis, clone COL06920 | 0.672581 | 0.000223 |
| 220531_at | FLJ14126 | hypothetical protein FLJ14126 | 0.672223 | 0.002803 |
| 206008_at | TGM1 | transglutaminase 1 (K polypeptide epidermal type I, protein- | 0.671929 | 0.000167 |
| | | glutamine-gamma-glutamyltransferase) | | |
| 212173_at | AK2 | adenylate kinase 2 | 0.671486 | 0.004892 |
| 218939_at | LETM1 | leucine zipper-EF-hand containing transmembrane protein 1 | 0.671473 | 0.009256 |
| 207299_s_at | GRM1 | glutamate receptor, metabotropic 1 | 0.670659 | 0.005754 |
| 205999_x_at | CYP3A4 | cytochrome P450, family 3, subfamily A, polypeptide 4 | 0.67042 | 0.004553 |
| 210773_s_at | FPRL1 | formyl peptide receptor-like 1 /// formyl peptide receptor-like 1 | 0.67022 | 0.002169 |
| 204680_s_at | RAPGEF5 | Rap guanine nucleotide exchange factor (GEF) 5 | 0.670041 | 0.006308 |
| 208521_at | OR5I1 | olfactory receptor, family 5, subfamily I, member 1 | 0.67003 | 0.00512 |
| 212092_at | PEG10 | paternally expressed 10 | 0.669851 | 0.002749 |
| 207921_x_at | PAX8 | paired box gene 8 | 0.669082 | 0.006122 |
| 216340_s_at | CYP2A7P1 | cytochrome P450, family 2, subfamily A, polypeptide 7 | 0.668582 | 0.002646 |
| | | pseudogene 1 | | |
| 214405_at | CUGBP2 | CUG triplet repeat, RNA binding protein 2 | 0.667683 | 0.000104 |
| 216270_at | ILVBL | ilvB (bacterial acetolactate synthase)-like | 0.667477 | 0.007231 |
| 201815_s_at | TBC1D5 | TBC1 domain family, member 5 | 0.667378 | 0.009907 |
| 204705_x_at | ALDOB | aldolase B, fructose-bisphosphate | 0.666853 | 0.009626 |
| 208483_x_at | KRTHA3A | keratin, hair, acidic, 3A | 0.666273 | 0.000862 |
| 216970_at | RGS7 | regulator of G-protein signalling 7 | 0.665812 | 0.000289 |
| 214628_at | NHLH1 | nescient helix loop helix 1 | 0.665803 | 0.007019 |
| 208365_s_at | GRK4 | G protein-coupled receptor kinase 4 | 0.664082 | 0.003763 |
| 217490_at | — | — | 0.663949 | 0.004796 |
| 208585_at | BTN2A3 | butyrophilin, subfamily 2, member A3 | 0.662932 | 0.009763 |
| 214135_at | CLDN18 | claudin 18 | 0.662862 | 0.009927 |
| 221421_s_at | ADAMTS12 | ADAM metallopeptidase with thrombospondin type 1 motif, 12 /// | 0.662405 | 0.008913 |
| | | ADAM metallopeptidase with thrombospondin type 1 motif, 12 | | |
| 222370_x_at | LOC388906 | Hypothetical gene supported by BC039496 | 0.661134 | 0.000577 |
| 207429_at | SLC22A2 | solute carrier family 22 (organic cation transporter), member 2 | 0.661115 | 0.005383 |
| 217183_at | LDLR | low density lipoprotein receptor (familial hypercholesterolemia) | 0.660799 | 0.00832 |
| 210888_s_at | ITIH1 | inter-alpha (globulin) inhibitor H1 | 0.660776 | 0.000405 |
| 38487_at | STAB1 | stabilin 1 | 0.659954 | 0.002558 |
| 207316_at | HAS1 | hyaluronan synthase 1 | 0.659204 | 0.000687 |
| 219535_at | HUNK | hormonally upregulated Neu-associated kinase | 0.658977 | 0.001516 |
| 216725_at | DCAMKL2 | Doublecortin and CaM kinase-like 2 | 0.65883 | 0.002664 |
| 220599_s_at | CARD14 | caspase recruitment domain family, member 14 | 0.658331 | 0.006057 |
| 207373_at | HOXD10 | homeo box D10 | 0.657965 | 0.001713 |
| 206873_at | CA6 | carbonic anhydrase VI | 0.657297 | 0.000275 |
| 203564_at | FANCG | Fanconi anemia, complementation group G | 0.656636 | 0.00802 |
| 207803_s_at | CSN3 | casein kappa | 0.656344 | 0.000671 |
| 222245_s_at | FER1L4 | fer-1-like 4 (C. elegans) | 0.65633 | 0.001014 |
| 220208_at | ADAMTS13 | ADAM metallopeptidase with thrombospondin type 1 motif, 13 | 0.65628 | 0.000316 |
| 215479_at | SEMA6A | Sema domain, transmembrane domain (TM), and cytoplasmic | 0.655939 | 0.000832 |
| | | domain, (semaphorin) 6A | | |
| 219452_at | DPEP2 | dipeptidase 2 | 0.65569 | 0.007827 |
| 219369_s_at | OTUB2 | OTU domain, ubiquitin aldehyde binding 2 | 0.655527 | 0.001941 |
| 210240_s_at | CDKN2D | cyclin-dependent kinase inhibitor 2D (p19, inhibits CDK4) | 0.655421 | 0.001936 |
| 219144_at | DUSP26 | dual specificity phosphatase 26 (putative) | 0.655364 | 0.007142 |
| 207143_at | CDK6 | cyclin-dependent kinase 6 | 0.65494 | 0.003864 |
| 210784_x_at | LILRB2 /// | leukocyte immunoglobulin-like receptor, subfamily B (with TM | 0.654114 | 0.008924 |
| | LILRB3 | and ITIM domains), member 2 /// leukocyte immunoglobulin-like | | |
| | | receptor, subfamily B (with TM and ITIM domains), member 3 | | |
| 39318_at | TCL1A | T-cell leukemia/lymphoma 1A | 0.65396 | 0.000332 |
| 216937_s_at | RS1 | retinoschisis (X-linked, juvenile) 1 | 0.653486 | 0.001534 |
| 205985_x_at | CLCNKB | chloride channel Kb | 0.653162 | 0.005956 |
| 212821_at | PLEKHG3 | pleckstrin homology domain containing, family G (with RhoGef | 0.652689 | 0.004061 |
| | | domain) member 3 | | |
| 220683_at | RDH8 | retinol dehydrogenase 8 (all-trans) | 0.651726 | 0.003389 |
| 215128_at | — | CDNA FLJ11682 fis, clone HEMBA1004880 | 0.651235 | 0.004292 |
| 210660_at | LILRA1 | leukocyte immunoglobulin-like receptor, subfamily A (with TM | 0.651118 | 7.02E−05 |
| | | domain), member 1 /// leukocyte immunoglobulin-like receptor, | | |
| | | subfamily A (with TM domain), member 1 | | |
| 207203_s_at | NR1I2 | nuclear receptor subfamily 1, group I, member 2 | 0.651057 | 0.008407 |
| 210349_at | CAMK4 | calcium/calmodulin-dependent protein kinase IV | 0.650668 | 0.002781 |
| 217272_s_at | SERPINB13 | serpin peptidase inhibitor, clade B (ovalbumin), member 13 | 0.650189 | 5.23E−05 |
| 207433_at | IL10 | interleukin 10 | 0.649524 | 0.009087 |
| 216203_at | SPTLC2 | serine palmitoyltransferase, long chain base subunit 2 | 0.648129 | 0.006588 |
| 211127_x_at | EDA | ectodysplasin A | 0.647757 | 3.94E−05 |
| 221106_at | SLC22A17 | solute carrier family 22 (organic cation transporter), member 17 | 0.647437 | 0.009469 |
| 214535_s_at | ADAMTS2 | ADAM metallopeptidase with thrombospondin type 1 motif, 2 | 0.647426 | 0.006177 |
| 207194_s_at | ICAM4 | intercellular adhesion molecule 4, Landsteiner-Wiener blood | 0.647368 | 0.006822 |
| | | group | | |
| 205473_at | ATP6V1B1 | ATPase, H+ transporting, lysosomal 56/58 kDa, V1 subunit B, | 0.647213 | 0.001514 |
| | | isoform 1 (Renal tubular acidosis with deafness) | | |
| 220025_at | TBR1 | T-box, brain, 1 | 0.647042 | 0.000433 |
| 215015_at | LOC92558 | hypothetical protein LOC92558 | 0.647 | 0.008419 |
| 211807_x_at | PCDHGB5 | protocadherin gamma subfamily B, 5 | 0.645647 | 0.000406 |
| 206058_at | SLC6A12 | solute carrier family 6 (neurotransmitter transporter, | 0.645204 | 0.003786 |
| | | betaine/GABA), member 12 | | |
| 210318_at | RBP3 | retinol binding protein 3, interstitial | 0.644939 | 0.00617 |
| 213796_at | SPRR1A | small proline-rich protein 1A | 0.644378 | 0.002666 |
| 214883_at | THRA | thyroid hormone receptor, alpha (erythroblastic leukemia viral (verb- | 0.644145 | 0.001489 |
| | | a) oncogene homolog, avian) | | |
| 203861_s_at | ACTN2 | actinin, alpha 2 | 0.644141 | 0.009782 |
| 216663_s_at | ZMYND10 | zinc finger, MYND-type containing 10 | 0.643923 | 0.005071 |
| 217568_at | FAM12A | family with sequence similarity 12, member A | 0.642689 | 0.001106 |
| 217280_x_at | GABRA5 | gamma-aminobutyric acid (GABA) A receptor, alpha 5 | 0.64247 | 0.000332 |
| 217686_at | PTPN1 | protein tyrosine phosphatase, non-receptor type 1 | 0.642061 | 0.006585 |
| 204585_s_at | L1CAM | L1 cell adhesion molecule | 0.641976 | 0.005439 |
| 214393_at | LOC284062 | hypothetical protein LOC284062 | 0.641908 | 0.002076 |
| 211301_at | KCND3 | potassium voltage-gated channel, Shal-related subfamily, | 0.641723 | 0.006252 |
| | | member 3 | | |
| 210841_s_at | NRP2 | neuropilin 2 | 0.64116 | 0.006339 |
| 221446_at | ADAM30 | ADAM metallopeptidase domain 30 | 0.641056 | 0.001598 |
| 210933_s_at | FSCN1 | fascin homolog 1, actin-bundling protein (Strongylocentrotus | 0.639664 | 0.004596 |
| | | purpuratus) | | |
| 213985_s_at | C19orf6 | Chromosome 19 open reading frame 6 | 0.639385 | 0.009485 |
| 211624_s_at | DRD2 | dopamine receptor D2 /// dopamine receptor D2 | 0.63907 | 0.008961 |
| 222260_at | — | — | 0.638943 | 0.004649 |
| 216117_at | EXOSC2 | exosome component 2 | 0.638391 | 0.009761 |
| 216651_s_at | GAD2 | glutamate decarboxylase 2 (pancreatic islets and brain, 65 kDa) | 0.638329 | 0.00349 |
| 214655_at | GPR6 | G protein-coupled receptor 6 | 0.638125 | 0.001913 |
| 214478_at | SPP2 | secreted phosphoprotein 2, 24 kDa | 0.637981 | 0.009715 |
| 220488_s_at | BCAS3 | breast carcinoma amplified sequence 3 | 0.637797 | 0.00115 |
| 211190_x_at | CD84 | CD84 antigen (leukocyte antigen) | 0.637131 | 0.007378 |
| 220263_at | DAMS | SMAD in the antisense orientation | 0.636198 | 0.001032 |
| 211222_s_at | HAP1 | huntingtin-associated protein 1 (neuroan 1) | 0.635069 | 0.007545 |
| 215858_at | — | CDNA FLJ12301 fis, clone MAMMA1001858 | 0.635059 | 0.002254 |
| 217444_at | C20orf112 | Chromosome 20 open reading frame 112 | 0.635005 | 0.002125 |
| 205445_at | PRL | prolactin | 0.634649 | 0.008128 |
| 217667_at | — | — | 0.63437 | 0.003206 |
| 203811_s_at | DNAJB4 | DnaJ (Hsp40) homolog, subfamily B, member 4 | 0.633838 | 0.00937 |
| 210141_s_at | INHA | inhibin, alpha | 0.632752 | 0.007205 |
| 209995_s_at | TCL1A | T-cell leukemia/lymphoma 1A /// T-cell leukemia/lymphoma 1A | 0.632732 | 0.006924 |
| 221413_at | KCNAB3 | potassium voltage-gated channel, shaker-related subfamily, beta | 0.632716 | 0.00132 |
| | | member 3 | | |
| 220350_at | ZNF235 | zinc finger protein 235 | 0.632367 | 0.003665 |
| 213008_at | FLJ10719 | hypothetical protein FLJ10719 | 0.632358 | 0.002705 |
| 207612_at | WNT8B | wingless-type MMTV integration site family, member 8B | 0.631405 | 0.003129 |
| 215406_at | TRIO | Triple functional domain (PTPRF interacting) | 0.631368 | 3.81E−05 |
| 205587_at | FGFR1OP | FGFR1 oncogene partner | 0.631259 | 0.006694 |
| 220185_at | SPTBN4 | spectrin, beta, non-erythrocytic 4 | 0.630963 | 0.008977 |
| 217035_at | V-set and | Similar to Ig heavy chain V-I region HG3 precursor | 0.630534 | 0.002426 |
| | immunoglobulin | | | |
| | domain | | | |
| | containing 6 | | | |
| 221266_s_at | TM7SF4 | transmembrane 7 superfamily member 4 /// transmembrane 7 | 0.63045 | 0.00336 |
| | | superfamily member 4 | | |
| 210821_x_at | CENPA | centromere protein A, 17 kDa | 0.630091 | 0.00604 |
| 222015_at | CSNK1E | Casein kinase 1, epsilon | 0.629986 | 0.006464 |
| 221742_at | CUGBP1 | CUG triplet repeat, RNA binding protein 1 | 0.62962 | 0.003909 |
| 211135_x_at | LILRB2 /// | leukocyte immunoglobulin-like receptor, subfamily B (with TM | 0.629619 | 0.004875 |
| | LILRB3 | and ITIM domains), member 2 /// leukocyte immunoglobulin-like | | |
| | | receptor, subfamily B (with TM and ITIM domains), member 3 | | |
| 221384_at | UCP1 | uncoupling protein 1 (mitochondrial, proton carrier) | 0.629528 | 0.003518 |
| 216054_x_at | MYL4 | myosin, light polypeptide 4, alkali; atrial, embryonic | 0.629321 | 0.0095 |
| 206198_s_at | CEACAM7 | carcinoembryonic antigen-related cell adhesion molecule 7 | 0.628859 | 0.000456 |
| 214615_at | P2RY10 | purinergic receptor P2Y, G-protein coupled, 10 | 0.628501 | 0.001264 |
| 219635_at | ZNF606 | zinc finger protein 606 | 0.628266 | 0.005787 |
| 204933_s_at | TNFRSF11B | tumor necrosis factor receptor superfamily, member 11b | 0.628073 | 0.009766 |
| | | (osteoprotegerin) | | |
| 211863_x_at | HFE | hemochromatosis | 0.62797 | 0.001587 |
| 220593_s_at | FLJ20753 | hypothetical protein FLJ20753 | 0.627656 | 0.007883 |
| 217086_at | CHRNB3 | cholinergic receptor, nicotinic, beta polypeptide 3 | 0.62761 | 0.002219 |
| 206709_x_at | GPT | glutamic-pyruvate transaminase (alanine aminotransferase) | 0.627482 | 0.001786 |
| 221343_at | OR11A1 | olfactory receptor, family 11, subfamily A, member 1 | 0.627306 | 0.00126 |
| 208321_s_at | CABP1 | calcium binding protein 1 (calbrain) | 0.626533 | 0.003268 |
| 205789_at | CD1D | CD1D antigen, d polypeptide /// CD1D antigen, d polypeptide | 0.626018 | 0.00363 |
| 219404_at | EPS8L3 | EPS8-like 3 | 0.625934 | 0.002602 |
| 210647_x_at | PLA2G6 | phospholipase A2, group VI (cytosolic, calcium-independent) | 0.625339 | 0.006877 |
| 221292_at | PTCH2 | patched homolog 2 (Drosophila) | 0.625336 | 0.001284 |
| 213257_at | SARM1 | sterile alpha and TIR motif containing 1 | 0.62507 | 0.004291 |
| 213723_s_at | IDUA | Iduronidase, alpha-L- | 0.624433 | 0.005599 |
| 206062_at | GUCA1A | guanylate cyclase activator 1A (retina) | 0.624433 | 0.005125 |
| 220616_at | — | — | 0.624111 | 0.004223 |
| 203299_s_at | AP1S2 | adaptor-related protein complex 1, sigma 2 subunit | 0.622611 | 0.003786 |
| 205936_s_at | HK3 | hexokinase 3 (white cell) | 0.622537 | 0.001779 |
| 32625_at | NPR1 | natriuretic peptide receptor A/guanylate cyclase A | 0.621574 | 0.005781 |
| | | (atrionatriuretic peptide receptor A) | | |
| 216191_s_at | TRA@ /// TRD@ | T cell receptor alpha locus /// T cell receptor delta locus | 0.621343 | 0.001205 |
| 221668_s_at | DNAI2 | dynein, axonemal, intermediate polypeptide 2 | 0.620678 | 0.006114 |
| 214317_x_at | RPS9 | Ribosomal protein S9 | 0.62026 | 0.004392 |
| 213866_at | LOC201191 | hypothetical protein LOC201191 | 0.620043 | 0.001367 |
| 210637_at | TACR1 | tachykinin receptor 1 | 0.61959 | 0.003206 |
| 217723_x_at | — | — | 0.619563 | 0.001015 |
| 215841_at | GUCA1B | guanylate cyclase activator 1B (retina) | 0.618949 | 0.005487 |
| 65635_at | FLJ21865 | endo-beta-N-acetylglucosaminidase | 0.618868 | 0.004338 |
| 207270_x_at | CD300C | CD300C antigen | 0.618761 | 0.00465 |
| 207301_at | EFNA5 | ephrin-A5 | 0.61795 | 0.005435 |
| 205819_at | MARCO | macrophage receptor with collagenous structure /// macrophage | 0.617499 | 0.008831 |
| | | receptor with collagenous structure | | |
| 215885_at | — | — | 0.616977 | 0.002018 |
| 211460_at | TTTY9 /// TTY9 | testis-specific transcript, Y-linked 9 /// testis-specific transcript, Y- | 0.616537 | 0.001348 |
| | | linked 9, centromeric | | |
| 208592_s_at | CD1E | CD1E antigen, e polypeptide /// CD1E antigen, e polypeptide | 0.616321 | 0.004826 |
| 217135_x_at | — | — | 0.616148 | 0.007796 |
| 221611_s_at | PHF7 | PHD finger protein 7 | 0.615092 | 0.006569 |
| 207927_at | HTR7 | 5-hydroxytryptamine (serotonin) receptor 7 (adenylate cyclase- | 0.614367 | 0.006351 |
| | | coupled) | | |
| 215503_at | — | Serine protease inhibitor Kunitz type 1 (SPINT1) | 0.613802 | 0.00124 |
| 221623_at | BCAN | brevican | 0.612912 | 0.001198 |
| 213950_s_at | PPP3CC | Protein phosphatase 3 (formerly 2B), catalytic subunit, gamma | 0.612743 | 0.000199 |
| | | isoform (calcineurin A gamma) | | |
| 207755_at | — | — | 0.611983 | 0.001338 |
| 216965_x_at | SPG20 | spastic paraplegia 20, spartin (Troyer syndrome) | 0.611828 | 0.009612 |
| 215251_at | — | Similar to SH3 and multiple ankyrin repeat domains protein 2 | 0.611778 | 0.004974 |
| | | (Shank2) (Proline-rich synapse associated protein 1) (ProSAP1) | | |
| | | (Cortactin-binding protein 1) (CortBP1) (GKAP/SAPAP | | |
| | | interacting protein) (SPANK-3) | | |
| 208570_at | WNT1 | wingless-type MMTV integration site family, member 1 | 0.611661 | 0.008122 |
| 202408_s_at | PRPF31 | PRP31 pre-mRNA processing factor 31 homolog (yeast) | 0.61135 | 0.001575 |
| 216568_x_at | — | — | 0.611144 | 0.004441 |
| 204945_at | PTPRN | protein tyrosine phosphatase, receptor type, N | 0.610554 | 0.000631 |
| 216869_at | PDE1C | phosphodiesterase 1C, calmodulin-dependent 70 kDa | 0.610234 | 0.00822 |
| 210363_s_at | SCN2B | sodium channel, voltage-gated, type II, beta | 0.61014 | 0.001105 |
| 215705_at | PPP5C | protein phosphatase 5, catalytic subunit | 0.609351 | 0.004181 |
| 206305_s_at | C8A | complement component 8, alpha polypeptide | 0.608955 | 0.004941 |
| 217434_at | MC2R | melanocortin 2 receptor (adrenocorticotropic hormone) | 0.608871 | 0.007118 |
| 220406_at | TGFB2 | transforming growth factor, beta 2 | 0.60829 | 0.004133 |
| 217251_x_at | — | Clone ds1-1 immunoglobulin lambda chain VJ region, (IGL) | 0.607862 | 0.002746 |
| 222213_x_at | — | — | 0.607578 | 0.002609 |
| 47105_at | DUS2L | dihydrouridine synthase 2-like (SMM1, S. cerevisiae) | 0.607575 | 0.000847 |
| 208049_s_at | TACR1 | tachykinin receptor 1 | 0.607313 | 0.004046 |
| 213993_at | SPON1 | spondin 1, extracellular matrix protein | 0.606896 | 0.002538 |
| 211402_x_at | NR6A1 | nuclear receptor subfamily 6, group A, member 1 | 0.605736 | 0.004913 |
| 208588_at | FKSG2 | apoptosis inhibitor | 0.605522 | 0.009692 |
| 207083_s_at | CC2D1A | coiled-coil and C2 domain containing 1A | 0.605344 | 0.009042 |
| 207488_at | — | — | 0.605326 | 0.00153 |
| 222223_s_at | IL1F5 | interleukin 1 family, member 5 (delta) | 0.603558 | 0.003638 |
| 208023_at | TNFRSF4 | tumor necrosis factor receptor superfamily, member 4 | 0.603404 | 0.007414 |
| 210481_s_at | CLEC4M | C-type lectin domain family 4, member M | 0.602978 | 0.004123 |
| 207215_at | HS322B1A | hypothetical protein HS322B1A | 0.602251 | 0.002055 |
| 203993_x_at | C21orf2 | Chromosome 21 open reading frame 2 | 0.601152 | 0.006718 |
| 214292_at | ITGB4 | integrin, beta 4 | 0.599488 | 0.00067 |
| 45687_at | MGC3121 | hypothetical protein MGC3121 | 0.599175 | 0.005663 |
| 210348_at | 4-Sep | septin 4 | 0.598963 | 0.00847 |
| 220300_at | RGS3 | regulator of G-protein signalling 3 | 0.598707 | 0.005017 |
| 214075_at | NENF | neuron derived neurotrophic factor | 0.59817 | 0.008603 |
| 202849_x_at | GRK6 | G protein-coupled receptor kinase 6 | 0.598143 | 0.00911 |
| 210550_s_at | RASGRF1 | Ras protein-specific guanine nucleotide-releasing factor 1 | 0.59651 | 0.001138 |
| 208461_at | HIC1 | hypermethylated in cancer 1 | 0.596316 | 0.008114 |
| 36888_at | KIAA0841 | KIAA0841 | 0.595873 | 0.004555 |
| 219273_at | CCNK | cyclin K | 0.595401 | 0.003298 |
| 215448_at | KCNH7 | Potassium voltage-gated channel, subfamily H (eag-related), | 0.594725 | 0.00273 |
| | | member 7 | | |
| 219672_at | ERAF | erythroid associated factor | 0.594655 | 0.002988 |
| 221160_s_at | CABP3 /// CABP5 | calcium binding protein 3 /// calcium binding protein 5 | 0.594624 | 0.008229 |
| 207894_s_at | TCL6 | T-cell leukemia/lymphoma 6 | 0.594094 | 0.003606 |
| 205385_at | MDM2 | Mdm2, transformed 3T3 cell double minute 2, p53 binding | 0.593924 | 0.000771 |
| | | protein (mouse) | | |
| 209892_at | FUT4 | fucosyltransferase 4 (alpha (1,3) fucosyltransferase, myeloid- | 0.593762 | 0.009594 |
| | | specific) | | |
| 221319_at | PCDHB8 | protocadherin beta 8 | 0.593487 | 0.000153 |
| 201974_s_at | C7orf28A /// | chromosome 7 open reading frame 28A /// chromosome 7 open | 0.592952 | 0.007782 |
| | C7orf28B /// | reading frame 28B /// MGC72080 pseudogene | | |
| | MGC72080 | | | |
| 222364_at | — | — | 0.592852 | 3.10E−06 |
| 208479_at | KCNA1 | potassium voltage-gated channel, shaker-related subfamily, | 0.59255 | 0.005249 |
| | | member 1 (episodic ataxia with myokymia) | | |
| 219486_at | DUS2L | dihydrouridine synthase 2-like (SMM1, S. cerevisiae) | 0.592424 | 0.005128 |
| 203701_s_at | TRMT1 | TRM1 tRNA methyltransferase 1 homolog (S. cerevisiae) | 0.592209 | 0.008997 |
| 215311_at | — | MRNA full length insert cDNA clone EUROIMAGE 21920 | 0.59055 | 0.007108 |
| 208077_at | C9orf38 | chromosome 9 open reading frame 38 | 0.589884 | 0.006422 |
| 205563_at | KISS1 | KiSS-1 metastasis-suppressor | 0.589787 | 0.000333 |
| 210848_at | — | — | 0.587265 | 0.003366 |
| 214541_s_at | QKI | quaking homolog, KH domain RNA binding (mouse) | 0.587238 | 0.005989 |
| 38158_at | ESPL1 | extra spindle poles like 1 (S. cerevisiae) | 0.587158 | 0.004525 |
| 208204_s_at | CAV3 | caveolin 3 | 0.586018 | 0.008961 |
| 208402_at | IL17 | interleukin 17 (cytotoxic T-lymphocyte-associated serine | 0.585653 | 0.003654 |
| | | esterase 8) | | |
| 215509_s_at | BUB1 | BUB1 budding uninhibited by benzimidazoles 1 homolog (yeast) | 0.5854 | 0.000708 |
| 217076_s_at | — | — | 0.584745 | 0.006329 |
| 205670_at | GAL3ST1 | galactose-3-O-sulfotransferase 1 | 0.583995 | 0.001825 |
| 206946_at | HCN4 | hyperpolarization activated cyclic nucleotide-gated potassium | 0.58382 | 0.003471 |
| | | channel 4 | | |
| 220043_s_at | MFI2 | antigen p97 (melanoma associated) identified by monoclonal | 0.583669 | 0.003066 |
| | | antibodies 133.2 and 96.5 | | |
| 211082_x_at | MARK2 | MAP/microtubule affinity-regulating kinase 2 /// MAP/microtubule | 0.583576 | 0.003936 |
| | | affinity-regulating kinase 2 | | |
| 213362_at | PTPRD | Protein tyrosine phosphatase, receptor type, D | 0.582878 | 0.008042 |
| 220518_at | ABI3BP | ABI gene family, member 3 (NESH) binding protein | 0.582699 | 0.00063 |
| 215056_at | — | CDNA FLJ42813 fis, clone BRCAN2012355 | 0.582688 | 0.003358 |
| 217308_at | OR1F2 | olfactory receptor, family 1, subfamily F, member 2 | 0.581504 | 0.002035 |
| 214735_at | PIP3-E | phosphoinositide-binding protein PIP3-E | 0.580796 | 0.003536 |
| 204932_at | TNFRSF11B | tumor necrosis factor receptor superfamily, member 11b | 0.58071 | 0.001173 |
| | | (osteoprotegerin) | | |
| 220310_at | TUBAL3 | tubulin, alpha-like 3 | 0.579534 | 0.003962 |
| 49327_at | SIRT3 | sirtuin (silent mating type information regulation 2 homolog) 3 | 0.578671 | 0.006642 |
| | | (S. cerevisiae) | | |
| 209893_s_at | FUT4 | fucosyltransferase 4 (alpha (1,3) fucosyltransferase, myeloid- | 0.577649 | 0.009857 |
| | | specific) | | |
| 221107_at | CHRNA9 | cholinergic receptor, nicotinic, alpha polypeptide 9 | 0.576864 | 0.007318 |
| 208408_at | PTN | pleiotrophin (heparin binding growth factor 8, neurite growth- | 0.576188 | 0.006901 |
| | | promoting factor 1) | | |
| 206647_at | HBZ | hemoglobin, zeta /// hemoglobin, zeta | 0.573944 | 0.001229 |
| 207195_at | CNTN6 | contactin 6 | 0.573912 | 0.001116 |
| 209810_at | SFTPB | surfactant, pulmonary-associated protein B | 0.573774 | 0.00395 |
| 59433_at | LOC286434 | hypothetical protein LOC286434 | 0.573142 | 0.008378 |
| 211510_s_at | CRHR2 | corticotropin releasing hormone receptor 2 | 0.571793 | 0.004504 |
| 216664_at | TTTY6 | Testis-specific transcript, Y-linked 6B | 0.571502 | 0.000258 |
| 207230_at | CDON | Cdon homolog (mouse) | 0.571185 | 0.002757 |
| 205227_at | IL1RAP | interleukin 1 receptor accessory protein | 0.570456 | 0.009194 |
| 219612_s_at | FGG | fibrinogen gamma chain | 0.569725 | 0.00635 |
| 211553_x_at | APAF1 | apoptotic peptidase activating factor | 0.569634 | 0.002359 |
| 206519_x_at | — | — | 0.569361 | 8.32E−05 |
| 210770_s_at | CACNA1A | calcium channel, voltage-dependent, P/Q type, alpha 1A subunit | 0.568927 | 0.005628 |
| 220438_at | QPCTL | glutaminyl-peptide cyclotransferase-like | 0.568565 | 0.005419 |
| 206470_at | PLXNC1 | plexin C1 | 0.568486 | 0.006338 |
| 33778_at | TBC1D22A | TBC1 domain family, member 22A | 0.56835 | 0.007009 |
| 216144_at | — | MRNA; cDNA DKFZp434K1126 (from clone DKFZp434K1126) | 0.568339 | 0.002858 |
| 214503_x_at | GPR135 | G protein-coupled receptor 135 | 0.5679 | 0.007842 |
| 218860_at | NOC4L | nucleolar complex associated 4 homolog (S. cerevisiae) | 0.567886 | 0.008642 |
| 216463_at | — | MRNA; cDNA DKFZp434P2435 (from clone DKFZp434P2435) | 0.567236 | 0.000328 |
| 205015_s_at | TGFA | transforming growth factor, alpha | 0.566861 | 0.00543 |
| 210894_s_at | CEP2 | centrosomal protein 2 | 0.566449 | 0.00108 |
| 221337_s_at | ADAM29 | ADAM metallopeptidase domain 29 | 0.56616 | 0.005438 |
| 222050_at | FXR2 | Fragile X mental retardation, autosomal homolog 2 | 0.566051 | 0.009145 |
| 216490_x_at | LOC442175 | similar to dJ408B20.3 (novel protein similar to 60S acidic | 0.564668 | 3.54E−05 |
| | | ribosomal protein P2 (RPLP2)) | | |
| 207800_at | AKAP5 | A kinase (PRKA) anchor protein 5 | 0.563558 | 0.000584 |
| 217174_s_at | APC2 | adenomatosis polyposis coli 2 | 0.563107 | 0.002844 |
| 215319_at | ATP8B3 | ATPase, Class I, type 8B, member 3 | 0.562293 | 0.000492 |
| 222194_at | — | LOC441340 | 0.561939 | 0.00039 |
| 206229_x_at | PAX2 | paired box gene 2 | 0.561416 | 0.008025 |
| 215232_at | KIAA0672 | KIAA0672 gene product | 0.561288 | 0.006133 |
| 221468_at | XCR1 | chemokine (C motif) receptor 1 | 0.559982 | 0.001816 |
| 207033_at | GIF | gastric intrinsic factor (vitamin B synthesis) | 0.559472 | 0.002153 |
| 210295_at | MAGEA10 | melanoma antigen family A, 10 | 0.559326 | 0.00481 |
| 213442_x_at | SPDEF | SAM pointed domain containing ets transcription factor | 0.558993 | 0.008483 |
| 217265_at | PLLP | plasma membrane proteolipid (plasmolipin) | 0.558915 | 0.000801 |
| 207640_x_at | NTN2L | netrin 2-like (chicken) | 0.558788 | 0.00901 |
| 204465_s_at | INA | internexin neuronal intermediate filament protein, alpha | 0.558478 | 0.00847 |
| 216900_s_at | CHRNA4 | cholinergic receptor, nicotinic, alpha polypeptide 4 | 0.55825 | 0.005206 |
| 220339_s_at | TPSG1 | tryptase gamma 1 | 0.558183 | 0.007343 |
| 216846_at | IGLJ3 | Immunoglobulin lambda joining 3 | 0.55741 | 0.002518 |
| 216706_x_at | IGHD /// IGHG1 | immunoglobulin heavy constant delta /// immunoglobulin heavy | 0.556765 | 0.002512 |
| | /// IGHM /// | constant gamma 1 (G1m marker) /// immunoglobulin heavy | | |
| | MGC27165 | constant mu /// hypothetical protein MGC27165 | | |
| 205477_s_at | AMBP | alpha-1-microglobulin/bikunin precursor | 0.55564 | 0.002929 |
| 207731_at | — | — | 0.555337 | 0.002242 |
| 217078_s_at | CD300A | CD300A antigen | 0.555085 | 0.001646 |
| 221678_at | ABHD6 | abhydrolase domain containing 6 | 0.554718 | 0.0014 |
| 201740_at | NDUFS3 | NADH dehydrogenase (ubiquinone) Fe—S protein 3, 30 kDa | 0.554653 | 0.008695 |
| | | (NADH-coenzyme Q reductase) | | |
| 207454_at | GRIK3 | glutamate receptor, ionotropic, kainate 3 | 0.554453 | 0.003656 |
| 206411_s_at | ABL2 | v-abl Abelson murine leukemia viral oncogene homolog 2 (arg, | 0.553519 | 0.004637 |
| | | Abelson-related gene) | | |
| 221135_s_at | HT001 | HT001 protein | 0.553333 | 0.002145 |
| 211260_at | BMP7 | bone morphogenetic protein 7 (osteogenic protein 1) | 0.552383 | 0.007889 |
| 217114_at | ASCC3L1 | activating signal cointegrator 1 complex subunit 3-like 1 | 0.551896 | 0.005451 |
| 216303_s_at | MTMR1 | myotubularin related protein 1 | 0.549384 | 0.005959 |
| 205785_at | ITGAM | integrin, alpha M (complement component receptor 3, alpha; also | 0.549087 | 0.009891 |
| | | known as CD11b (p170), macrophage antigen alpha | | |
| | | polypeptide) /// integrin, alpha M (complement component | | |
| | | receptor 3, alpha; also known as CD11b (p170), macrophage | | |
| | | antigen alpha polypeptide) | | |
| 216673_at | TTTY1 | testis-specific transcript, Y-linked 1 | 0.548649 | 0.003036 |
| 210215_at | TFR2 | transferrin receptor 2 | 0.548623 | 0.004671 |
| 211661_x_at | PTAFR | platelet-activating factor receptor /// platelet-activating factor | 0.548494 | 0.006088 |
| | | receptor | | |
| 207924_x_at | PAX8 | paired box gene 8 | 0.546927 | 0.001416 |
| 220828_s_at | FLJ11292 | hypothetical protein FLJ11292 | 0.546658 | 0.001122 |
| 220693_at | HSPC048 | HSPC048 protein | 0.545684 | 0.005326 |
| 220863_at | MIP | major intrinsic protein of lens fiber | 0.545251 | 0.00387 |
| 220209_at | PYY2 | peptide YY, 2 (seminalplasmin) | 0.544447 | 0.006375 |
| 202685_s_at | AXL | AXL receptor tyrosine kinase | 0.543616 | 0.00483 |
| 35160_at | LDB1 | LIM domain binding 1 | 0.543069 | 0.00939 |
| 217385_at | — | — | 0.543059 | 0.001748 |
| 206164_at | CLCA2 | chloride channel, calcium activated, family member 2 | 0.542638 | 0.000997 |
| 210395_x_at | MYL4 | myosin, light polypeptide 4, alkali; atrial, embryonic | 0.542577 | 0.008382 |
| 222384_at | DKFZP564C196 | DKFZP564C196 protein | 0.542523 | 0.002015 |
| 210914_at | — | — | 0.542031 | 0.001128 |
| 219592_at | MCPH1 | microcephaly, primary autosomal recessive 1 | 0.541931 | 0.003706 |
| 220469_at | COPE | coatomer protein complex, subunit epsilon | 0.541034 | 0.006578 |
| 220429_at | NDST3 | N-deacetylase/N-sulfotransferase (heparan glucosaminyl) 3 | 0.540881 | 0.009309 |
| 208342_x_at | CSH2 | chorionic somatomammotropin hormone 2 | 0.540756 | 0.006221 |
| 220325_at | TAF7L | TAF7-like RNA polymerase II, TATA box binding protein (TBP)- | 0.540362 | 0.006113 |
| | | associated factor, 50 kDa | | |
| 220479_at | PRO0132 | PRO0132 protein | 0.53925 | 0.000824 |
| 206907_at | TNFSF9 | tumor necrosis factor (ligand) superfamily, member 9 | 0.538952 | 0.003153 |
| 201355_s_at | BAZ2A | bromodomain adjacent to zinc finger domain, 2A | 0.538397 | 0.009457 |
| 220829_s_at | B3GALT1 | UDP-Gal:betaGlcNAc beta 1,3-galactosyltransferase, | 0.536755 | 0.006489 |
| | | polypeptide 1 | | |
| 216403_at | — | CDNA clone MGC: 43345 IMAGE: 5266022 | 0.536098 | 0.006951 |
| 206219_s_at | VAV1 | vav 1 oncogene | 0.536011 | 0.006448 |
| 215874_at | SIAT4A | ST3 beta-galactoside alpha-2,3-sialyltransferase 1 | 0.535488 | 0.006241 |
| 220515_at | DUSP21 | dual specificity phosphatase 21 | 0.535001 | 0.009688 |
| 207794_at | CCR2 | chemokine (C-C motif) receptor 2 /// chemokine (C-C motif) | 0.534671 | 0.001444 |
| | | receptor 2 | | |
| 205154_at | LRRN5 | leucine rich repeat neuronal 5 | 0.534503 | 0.009075 |
| 219107_at | BCAN | brevican | 0.534363 | 0.009847 |
| 208428_at | TAP2 | transporter 2, ATP-binding cassette, sub-family B (MDR/TAP) | 0.532656 | 0.00402 |
| 207710_at | LCE2B | late cornified envelope 2B | 0.531796 | 0.002212 |
| 208339_at | XKRY /// XKRY2 | X Kell blood group precursor-related, Y-linked /// X Kell blood | 0.531381 | 0.006964 |
| | | group precursor-related, Y-linked 2 | | |
| 219945_at | DDX25 | DEAD (Asp-Glu-Ala-Asp) box polypeptide 25 | 0.530814 | 0.001354 |
| 210575_at | NUDC | nuclear distribution gene C homolog (A. nidulans) | 0.530429 | 0.003926 |
| 207587_at | CRYGA | crystallin, gamma A | 0.529813 | 0.00288 |
| 217038_at | — | — | 0.529219 | 0.009708 |
| 214981_at | POSTN | Periostin, osteoblast specific factor | 0.528711 | 5.28E−07 |
| AFFX-ThrX-5_at | — | — | 0.528669 | 0.006018 |
| 214031_s_at | KRT7 | Keratin 7 | 0.528565 | 0.003093 |
| 216788_at | — | CDNA: FLJ21911 fis, clone HEP03855 | 0.528291 | 0.001129 |
| 203684_s_at | BCL2 | B-cell CLL/lymphoma 2 | 0.527834 | 0.00326 |
| 214346_at | C1QL1 | Complement component 1, q subcomponent-like 1 | 0.527031 | 0.006684 |
| 214689_at | PAPPA2 | pappalysin 2 | 0.525972 | 0.009606 |
| 220383_at | ABCG5 | ATP-binding cassette, sub-family G (WHITE), member 5 (sterolin | 0.523406 | 0.002407 |
| | | 1) | | |
| 222215_at | FLJ10815 | amino acid transporter | 0.523224 | 0.002118 |
| 219391_at | BPNT1 | 3′(2′),5′-bisphosphate nucleotidase 1 | 0.523005 | 0.000582 |
| 31807_at | DDX49 | DEAD (Asp-Glu-Ala-Asp) box polypeptide 49 | 0.522692 | 0.007755 |
| 206568_at | TNP1 | transition protein 1 (during histone to protamine replacement) | 0.517882 | 0.006294 |
| 206610_s_at | F11 | coagulation factor XI (plasma thromboplastin antecedent) | 0.517517 | 0.003981 |
| 219912_s_at | ENPP3 | ectonucleotide pyrophosphatase/phosphodiesterase 3 | 0.51727 | 0.000826 |
| 216342_x_at | — | — | 0.517105 | 0.008785 |
| 210959_s_at | SRD5A1 | steroid-5-alpha-reductase, alpha polypeptide 1 (3-oxo-5 alpha- | 0.516774 | 0.00461 |
| | | steroid delta 4-dehydrogenase alpha 1) | | |
| 207797_s_at | LRP2BP | LRP2 binding protein | 0.514998 | 0.004767 |
| 204296_at | DCTN1 | dynactin 1 (p150, glued homolog, Drosophila) | 0.514549 | 0.002948 |
| 215593_at | GCNT2 | Glucosaminyl (N-acetyl) transferase 2, I-branching enzyme | 0.513994 | 0.008838 |
| 205869_at | PRSS1 | protease, serine, 1 (trypsin 1) | 0.513576 | 0.001762 |
| 220856_x_at | — | — | 0.512404 | 0.004678 |
| 211927_x_at | EEF1G | eukaryotic translation elongation factor 1 gamma | 0.512289 | 0.00134 |
| 212212_s_at | DKFZP586J0619 | DKFZP586J0619 protein | 0.505998 | 0.007375 |
| 215972_at | — | Clone 24820 mRNA sequence | 0.504998 | 0.005462 |
| 206283_s_at | TAL1 | T-cell acute lymphocytic leukemia 1 | 0.503202 | 0.002938 |
| 210464_at | DKFZP434F122 | DKFZP434F122 protein | 0.503043 | 0.000257 |
| 207293_s_at | AGTR2 | angiotensin II receptor, type 2 | 0.502279 | 0.006392 |
| 215928_at | ABLIM2 | Actin binding LIM protein family, member 2 | 0.501773 | 0.003583 |
| 205959_at | MMP13 | matrix metallopeptidase 13 (collagenase 3) /// matrix | 0.501616 | 0.003445 |
| | | metallopeptidase 13 (collagenase 3) | | |
| 205638_at | BAI3 | brain-specific angiogenesis inhibitor 3 | 0.501583 | 0.006815 |
| 214218_s_at | XIST | X (inactive)-specific transcript | 0.501335 | 0.007846 |
| 216414_at | — | — | 0.500838 | 0.007487 |
| 206408_at | LRRTM2 | leucine rich repeat transmembrane neuronal 2 | 0.500571 | 0.005991 |
| 221412_at | VN1R1 | vomeronasal 1 receptor 1 | 0.499936 | 0.007492 |
| 204551_s_at | AHSG | alpha-2-HS-glycoprotein | 0.499858 | 0.00195 |
| 216715_at | — | — | 0.498304 | 0.001478 |
| 207783_x_at | HUWE1 | HECT, UBA and WWE domain containing 1 | 0.497568 | 0.000327 |
| 220762_s_at | GNB1L | guanine nucleotide binding protein (G protein), beta polypeptide | 0.497334 | 0.00341 |
| | | 1-like | | |
| 211095_at | NF1 | neurofibromin 1 (neurofibromatosis, von Recklinghausen | 0.496407 | 0.00303 |
| | | disease, Watson disease) | | |
| 207113_s_at | TNF | tumor necrosis factor (TNF superfamily, member 2) | 0.495712 | 0.007599 |
| 211803_at | CDK2 | cyclin-dependent kinase 2 | 0.494575 | 0.003554 |
| 217177_s_at | PTPRB | Protein tyrosine phosphatase, receptor type, B | 0.49433 | 0.002134 |
| 213790_at | ADAM12 | ADAM metallopeptidase domain 12 (meltrin alpha) | 0.493682 | 0.001824 |
| 220848_x_at | OBP2A | odorant binding protein 2A | 0.492329 | 0.007613 |
| 208533_at | SOX1 | SRY (sex determining region Y)-box 1 | 0.491415 | 0.004709 |
| 216543_at | ATXN3L | Ataxin 3-like | 0.490895 | 0.001243 |
| 220539_at | C10orf92 | chromosome 10 open reading frame 92 | 0.490415 | 0.001845 |
| 219574_at | 1-Mar | membrane-associated ring finger (C3HC4) 1 | 0.489991 | 0.003456 |
| 210420_at | SLC24A1 | solute carrier family 24 (sodium/potassium/calcium exchanger), | 0.489204 | 0.005491 |
| | | member 1 | | |
| 221157_s_at | FBXO24 | F-box protein 24 | 0.488886 | 0.009943 |
| 213522_s_at | SLC16A3 | Solute carrier family 16 (monocarboxylic acid transporters), | 0.487437 | 0.002822 |
| | | member 3 | | |
| 208443_x_at | SHOX2 | short stature homeobox 2 | 0.487165 | 0.001235 |
| 206454_s_at | RHO | rhodopsin (opsin 2, rod pigment) (retinitis pigmentosa 4, | 0.487143 | 0.006863 |
| | | autosomal dominant) | | |
| 214242_at | MAN1A2 | Mannosidase, alpha, class 1A, member 2 | 0.486975 | 0.008671 |
| 216328_at | SIGLEC8 | sialic acid binding Ig-like lectin 8 | 0.485087 | 0.000897 |
| 214865_at | DOT1L | DOT1-like, histone H3 methyltransferase (S. cerevisiae) | 0.484427 | 0.006673 |
| 214856_at | SPTBN1 | spectrin, beta, non-erythrocytic 1 | 0.484266 | 0.002145 |
| 201978_s_at | KIAA0141 | KIAA0141 | 0.484076 | 0.009636 |
| 217290_at | — | — | 0.483435 | 0.00094 |
| 91580_at | HT017 | HT017 protein | 0.482078 | 0.004105 |
| 206112_at | ANKRD7 | ankyrin repeat domain 7 | 0.4805 | 0.002927 |
| 207546_at | ATP4B | ATPase, H+/K+ exchanging, beta polypeptide | 0.478202 | 0.00138 |
| 214963_at | NUP160 | nucleoporin 160 kDa | 0.475484 | 0.00253 |
| 206651_s_at | CPB2 | carboxypeptidase B2 (plasma, carboxypeptidase U) | 0.475279 | 0.007868 |
| 206810_at | ZNF44 | zinc finger protein 44 (KOX 7) | 0.474827 | 0.001909 |
| 214371_at | TSSK2 | testis-specific serine kinase 2 | 0.473235 | 0.009611 |
| 206476_s_at | NOVA2 | neuro-oncological ventral antigen 2 | 0.470506 | 0.001733 |
| 219722_s_at | GDPD3 | glycerophosphodiester phosphodiesterase domain containing 3 | 0.469645 | 0.005162 |
| 205626_s_at | CALB1 | calbindin 1, 28 kDa | 0.468146 | 0.000913 |
| 206815_at | SPAG8 | sperm associated antigen 8 | 0.4679 | 0.006234 |
| 216362_at | — | — | 0.466559 | 0.006444 |
| 208387_s_at | MMP24 | matrix metallopeptidase 24 (membrane-inserted) | 0.466276 | 0.00844 |
| 207237_at | KCNA3 | potassium voltage-gated channel, shaker-related subfamily, | 0.46477 | 0.007942 |
| | | member 3 | | |
| 214602_at | COL4A4 | collagen, type IV, alpha 4 | 0.464434 | 0.005412 |
| 204317_at | GTSE1 /// | G-2 and S-phase expressed 1 /// hypothetical gene supported by | 0.464334 | 0.008554 |
| | LOC440834 | BC069212 | | |
| 216112_at | PKN2 | Protein kinase N2 | 0.464282 | 0.002523 |
| 208136_s_at | MGC3771 | hypothetical protein MGC3771 /// hypothetical protein MGC3771 | 0.463322 | 0.009919 |
| 216718_at | C1orf46 | chromosome 1 open reading frame 46 | 0.458812 | 0.000355 |
| 206462_s_at | NTRK3 | neurotrophic tyrosine kinase, receptor, type 3 | 0.458332 | 0.003598 |
| 216575_at | — | — | 0.45577 | 0.004607 |
| 221312_at | GLP2R | glucagon-like peptide 2 receptor | 0.455731 | 0.00488 |
| 216398_at | GYPB | glycophorin B (includes Ss blood group) | 0.455666 | 0.004296 |
| 220333_at | PAQRS | progestin and adipoQ receptor family member V | 0.45466 | 0.003078 |
| AFFX-r2- | — | — | 0.453581 | 0.005425 |
| Bs-phe-5_at | | | | |
| 216884_at | PTPN12 | protein tyrosine phosphatase, non-receptor type 12 | 0.452717 | 0.007439 |
| 206588_at | DAZL | deleted in azoospermia-like | 0.452577 | 0.00995 |
| 216440_at | RAB6IP2 | RAB6 interacting protein 2 | 0.448637 | 0.007838 |
| 201766_at | ELAC2 | elaC homolog 2 (E. coli) | 0.446211 | 0.002651 |
| 222051_s_at | E2F5 | E2F transcription factor 5, p130-binding | 0.444596 | 0.005832 |
| 206739_at | HOXC5 | homeo box C5 | 0.442931 | 0.004886 |
| 220402_at | P53AIP1 | p53-regulated apoptosis-inducing protein 1 | 0.441655 | 0.001081 |
| 220409_at | CAMSAP1 | calmodulin regulated spectrin-associated protein 1 | 0.43934 | 0.003158 |
| 217498_at | CIP29 | Cytokine induced protein 29 kDa | 0.438961 | 0.005393 |
| 216419_at | CROCC | ciliary rootlet coiled-coil, rootletin | 0.437163 | 0.007556 |
| 216436_at | PIK3R4 | Phosphoinositide-3-kinase, regulatory subunit 4, p150 | 0.436556 | 0.001587 |
| 221225_at | FLJ22955 | hypothetical protein FLJ22955 | 0.435122 | 0.003978 |
| 207089_at | NRAP | nebulin-related anchoring protein | 0.434536 | 0.008213 |
| 38398_at | MADD | MAP-kinase activating death domain | 0.429347 | 0.009013 |
| 210599_at | ZNF614 | zinc finger protein 614 | 0.426104 | 0.007446 |
| 216660_at | MYO7B | myosin VIIB | 0.424596 | 0.003008 |
| 215286_s_at | PHTF2 | putative homeodomain transcription factor 2 | 0.424586 | 0.009203 |
| 220891_at | FLJ35725 | hypothetical protein FLJ35725 | 0.424337 | 0.005043 |
| 217582_at | — | — | 0.424318 | 0.002472 |
| 205532_s_at | CDH6 | cadherin 6, type 2, K-cadherin (fetal kidney) | 0.42264 | 0.005493 |
| 215332_s_at | CD8B1 | CD8 antigen, beta polypeptide 1 (p37) | 0.418972 | 0.001269 |
| 205612_at | MMRN1 | multimerin 1 | 0.417059 | 0.006233 |
| 207694_at | POU3F4 | POU domain, class 3, transcription factor 4 | 0.414459 | 0.005265 |
| 215170_s_at | Cep152 | KIAA0912 protein | 0.410486 | 0.005335 |
| 216999_at | EPOR | erythropoietin receptor | 0.409791 | 0.005034 |
| 215730_at | VGLL1 | vestigial like 1 (Drosophila) | 0.409122 | 0.009912 |
| 206432_at | HAS2 | hyaluronan synthase 2 | 0.408043 | 0.006749 |
| 220854_at | — | — | 0.402616 | 0.008704 |
| 204913_s_at | SOX11 | SRY (sex determining region Y)-box 11 | 0.394032 | 0.004428 |
| 210548_at | CCL23 | chemokine (C-C motif) ligand 23 | 0.393334 | 0.009452 |
| 206637_at | P2RY14 | purinergic receptor P2Y, G-protein coupled, 14 | 0.39227 | 0.008732 |
| 211636_at | IGHA2 /// IGHG1 | immunoglobulin heavy constant alpha 2 (A2m marker) /// | 0.375105 | 0.005719 |
| | /// IGHG4 /// | immunoglobulin heavy constant alpha 2 (A2m marker) /// | | |
| | MGC27165 | immunoglobulin heavy constant gamma 1 (G1m marker) /// | | |
| | | immunoglobulin heavy constant gamma 1 (G1m marker) /// | | |
| | | immunoglobulin heavy constant gamma 4 (G4m marker) /// | | |
| | | immunoglobulin heavy constant gamma 4 (G4m marker) /// | | |
| | | hypothetical protein MGC27165 /// hypothetical protein | | |
| | | MGC27165 | | |
| 37004_at | SFTPB | surfactant, pulmonary-associated protein B | 0.372737 | 0.009383 |
| 215721_at | IGHG1 | immunoglobulin heavy constant gamma 1 (G1m marker) | 0.358356 | 0.003471 |
| 215993_at | ODZ2 | Odz, odd Oz/ten-m homolog 2 (Drosophila) | 0.358243 | 0.001751 |
| 213458_at | KIAA0974 | KIAA0974 | 0.356203 | 0.005884 |
| 215320_at | C9orf36 /// | chromosome 9 open reading frame 36 /// similar to chromosome | 0.346752 | 0.00244 |
| | LOC389730 /// | 9 open reading frame 36 /// similar to hypothetical protein /// | | |
| | LOC441448 /// | hypothetical LOC441452 | | |
| | DKFZp434M131 | | | |
| 217093_at | RNASE1 | ribonuclease, RNase A family, 1 (pancreatic) | 0.34182 | 0.007687 |
| 220229_s_at | AP4E1 | adaptor-related protein complex 4, epsilon 1 subunit | 0.340351 | 0.00864 |
| 214685_at | C4orf10 | chromosome 4 open reading frame 10 | −0.37907 | 0.004104 |
| 214764_at | KIAA0507 | KIAA0507 | −0.40738 | 0.004086 |
| 203830_at | NJMU-R1 | protein kinase Njmu-R1 | −0.42557 | 0.00978 |
| 217564_s_at | CPS1 | carbamoyl-phosphate synthetase 1, mitochondrial | −0.43025 | 0.004007 |
| 210598_at | — | — | −0.43636 | 0.00214 |
| 203840_at | BLZF1 | basic leucine zipper nuclear factor 1 (JEM-1) | −0.44266 | 0.004682 |
| 209148_at | RXRB | retinoid X receptor, beta | −0.44969 | 0.00571 |
| 220709_at | ZNF556 | zinc finger protein 556 | −0.45311 | 0.006952 |
| 200801_x_at | ACTB | actin, beta | −0.46595 | 0.003297 |
| 219560_at | FLJ21125 | hypothetical protein FLJ21125 | −0.47065 | 0.003378 |
| 216850_at | SNRPN | small nuclear ribonucleoprotein polypeptide N | −0.47712 | 0.005213 |
| 213867_x_at | ACTB | actin, beta | −0.48238 | 0.000108 |
| 205191_at | RP2 | retinitis pigmentosa 2 (X-linked recessive) | −0.49062 | 0.007308 |
| 209551_at | YIPF4 | Yip1 domain family, member 4 | −0.49484 | 0.005 |
| 208154_at | LOC51336 | mesenchymal stem cell protein DSCD28 | −0.50528 | 8.21E−05 |
| 215107_s_at | TTC22 | tetratricopeptide repeat domain 22 | −0.50711 | 0.001361 |
| 220462_at | TAIP-2 | TGF-beta induced apotosis protein 2 | −0.50883 | 0.006807 |
| 202298_at | NDUFA1 | NADH dehydrogenase (ubiquinone) 1 alpha subcomplex, 1, | −0.51043 | 0.003889 |
| | | 7.5 kDa | | |
| 201994_at | MORF4L2 | mortality factor 4 like 2 | −0.51419 | 0.004818 |
| 221156_x_at | CCPG1 | cell cycle progression 1 | −0.53005 | 0.005389 |
| 204407_at | TTF2 | transcription termination factor, RNA polymerase II | −0.53143 | 0.007447 |
| 64900_at | CHST5 /// | carbohydrate (N-acetylglucosamine 6-O) sulfotransferase 5 /// | −0.53252 | 0.008316 |
| | MGC15429 | hypothetical protein MGC15429 | | |
| 211304_x_at | KCNJ5 | potassium inwardly-rectifying channel, subfamily J, member 5 | −0.53432 | 0.002242 |
| 202903_at | LSM5 | LSM5 homolog, U6 small nuclear RNA associated (S. cerevisiae) | −0.53778 | 0.008794 |
| 221425_s_at | HBLD2 | HESB like domain containing 2 /// HESB like domain containing 2 | −0.54624 | 0.007175 |
| 218648_at | TORC3 | transducer of regulated cAMP response element-binding protein | −0.54707 | 0.005299 |
| | | (CREB) 3 | | |
| 200802_at | SARS | seryl-tRNA synthetase | −0.55149 | 0.00715 |
| 220367_s_at | SAP130 | mSin3A-associated protein 130 | −0.55521 | 0.009274 |
| 216306_x_at | PTBP1 | polypyrimidine tract binding protein 1 | −0.55647 | 0.003489 |
| 210286_s_at | SLC4A7 | solute carrier family 4, sodium bicarbonate cotransporter, | −0.5573 | 0.005855 |
| | | member 7 | | |
| 203462_x_at | EIF3S9 | eukaryotic translation initiation factor 3, subunit 9 eta, 116 kDa | −0.5573 | 0.008464 |
| 49077_at | PME-1 | protein phosphatase methylesterase-1 | −0.55892 | 0.002324 |
| 204075_s_at | GlyBP | glycine-, glutamate-, thienylcyclohexylpiperidine-binding protein | −0.56011 | 0.00169 |
| 210926_at | FKSG30 | actin-like protein | −0.56423 | 0.009825 |
| 200044_at | SFRS9 | splicing factor, arginine/serine-rich 9 /// splicing factor, | −0.56436 | 0.008848 |
| | | arginine/serine-rich 9 | | |
| 217659_at | KIAA0261 | KIAA0261 | −0.5738 | 0.008941 |
| 202758_s_at | RFXANK | regulatory factor X-associated ankyrin-containing protein | −0.57734 | 0.004711 |
| 203521_s_at | ZNF318 | zinc finger protein 318 | −0.57866 | 0.005221 |
| 217923_at | PEF1 | penta-EF-hand domain containing 1 | −0.58311 | 0.008409 |
| 220083_x_at | UCHL5 | ubiquitin carboxyl-terminal hydrolase L5 | −0.58367 | 0.003717 |
| 211358_s_at | CIZ1 | CDKN1A interacting zinc finger protein 1 | −0.58466 | 0.009976 |
| 209768_s_at | GP1BB | glycoprotein Ib (platelet), beta polypeptide | −0.58939 | 0.007031 |
| 216178_x_at | ITGB1 | integrin, beta 1 (fibronectin receptor, beta polypeptide, antigen | −0.59317 | 0.00416 |
| | | CD29 includes MDF2, MSK12) | | |
| 204208_at | RNGTT | RNA guanylyltransferase and 5′-phosphatase | −0.60123 | 0.002049 |
| 206356_s_at | GNAL | guanine nucleotide binding protein (G protein), alpha activating | −0.60253 | 0.007645 |
| | | activity polypeptide, olfactory type | | |
| 212376_s_at | EP400 | E1A binding protein p400 | −0.60585 | 0.001973 |
| 213686_at | — | Clone 24583 mRNA sequence | −0.60847 | 0.002594 |
| 215137_at | KIAA0508 | KIAA0508 protein | −0.61035 | 0.006535 |
| 207164_s_at | ZNF238 | zinc finger protein 238 | −0.61698 | 0.001899 |
| 203762_s_at | D2LIC | dynein 2 light intermediate chain | −0.62075 | 0.00397 |
| 219453_at | C16orf44 | chromosome 16 open reading frame 44 | −0.62805 | 0.001095 |
| 209533_s_at | PLAA | phospholipase A2-activating protein | −0.62938 | 0.005223 |
| 218514_at | FLJ10587 | hypothetical protein FLJ10587 | −0.63067 | 0.001076 |
| 212082_s_at | MYL6 | myosin, light polypeptide 6, alkali, smooth muscle and non- | −0.63518 | 0.001506 |
| | | muscle | | |
| 200978_at | MDH1 | malate dehydrogenase 1, NAD (soluble) | −0.63696 | 0.00294 |
| 214876_s_at | TUBGCP5 | tubulin, gamma complex associated protein 5 | −0.64125 | 0.008824 |
| 201972_at | ATP6V1A | ATPase, H+ transporting, lysosomal 70 kDa, V1 subunit A | −0.64351 | 0.007242 |
| 217917_s_at | DNCL2A | dynein, cytoplasmic, light polypeptide 2A | −0.65433 | 0.008307 |
| 219029_at | FLJ21657 | hypothetical protein FLJ21657 | −0.65453 | 0.002315 |
| 218318_s_at | NLK | nemo like kinase | −0.65533 | 0.004658 |
| 204991_s_at | NF2 | neurofibromin 2 (bilateral acoustic neuroma) | −0.65763 | 0.004678 |
| 216146_at | EIF4G3 | Eukaryotic translation initiation factor 4 gamma, 3 | −0.65785 | 0.005787 |
| 217411_s_at | RREB1 | ras responsive element binding protein 1 | −0.65849 | 0.002335 |
| 222212_s_at | LASS2 | LAG1 longevity assurance homolog 2 (S. cerevisiae) | −0.65853 | 0.006912 |
| 200818_at | ATP5O | ATP synthase, H+ transporting, mitochondrial F1 complex, O | −0.65984 | 0.00242 |
| | | subunit (oligomycin sensitivity conferring protein) | | |
| 202737_s_at | LSM4 | LSM4 homolog, U6 small nuclear RNA associated (S. cerevisiae) | −0.6604 | 0.005611 |
| 212146_at | PLEKHM2 | pleckstrin homology domain containing, family M (with RUN | −0.66098 | 0.004515 |
| | | domain) member 2 | | |
| 200066_at | IK | IK cytokine, down-regulator of HLA II /// IK cytokine, down- | −0.66131 | 0.006358 |
| | | regulator of HLA II | | |
| 212908_at | DNAJC16 | DnaJ (Hsp40) homolog, subfamily C, member 16 | −0.66237 | 0.007476 |
| 211950_at | RBAF600 | retinoblastoma-associated factor 600 | −0.66381 | 0.004816 |
| 218467_at | TNFSF5IP1 | tumor necrosis factor superfamily, member 5-induced protein 1 | −0.66451 | 0.006823 |
| 201180_s_at | GNAI3 | guanine nucleotide binding protein (G protein), alpha inhibiting | −0.66531 | 0.009823 |
| | | activity polypeptide 3 | | |
| 211017_s_at | NF2 | neurofibromin 2 (bilateral acoustic neuroma) | −0.67262 | 0.001037 |
| 212457_at | TFE3 | transcription factor binding to IGHM enhancer 3 | −0.67927 | 0.000156 |
| 209174_s_at | FLJ20259 | FLJ20259 protein | −0.67975 | 0.009051 |
| 214263_x_at | POLR2C | polymerase (RNA) II (DNA directed) polypeptide C, 33 kDa | −0.68031 | 0.007837 |
| 203654_s_at | COIL | coilin | −0.68038 | 0.003521 |
| 213127_s_at | MED8 | mediator of RNA polymerase II transcription, subunit 8 homolog | −0.6817 | 0.001107 |
| | | (yeast) | | |
| 215629_s_at | DLEU2 /// | deleted in lymphocytic leukemia, 2 /// BCMS upstream neighbor- | −0.68231 | 0.008964 |
| | BCMSUNL | like | | |
| 212221_x_at | IDS | iduronate 2-sulfatase (Hunter syndrome) | −0.68236 | 0.00954 |
| 216903_s_at | CBARA1 | calcium binding atopy-related autoantigen 1 | −0.68409 | 0.000426 |
| 218472_s_at | PELO | pelota homolog (Drosophila) | −0.68587 | 0.009589 |
| 220760_x_at | ZNF665 | zinc finger protein 665 | −0.68715 | 0.005059 |
| 202077_at | NDUFAB1 | NADH dehydrogenase (ubiquinone) 1, alpha/beta subcomplex, | −0.6891 | 0.004257 |
| | | 1, 8 kDa | | |
| 202471_s_at | IDH3G | isocitrate dehydrogenase 3 (NAD+) gamma | −0.68968 | 0.008398 |
| 221079_s_at | METTL2 /// | methyltransferase like 2 /// hypothetical protein FLJ12760 | −0.68983 | 6.97E−05 |
| | FLJ12760 | | | |
| 217835_x_at | C20orf24 | chromosome 20 open reading frame 24 | −0.69132 | 0.002311 |
| 209497_s_at | RBM30 | RNA binding motif protein 30 | −0.69412 | 0.003809 |
| 218012_at | TSPYL2 | TSPY-like 2 | −0.69419 | 0.001275 |
| 212112_s_at | STX12 | syntaxin 12 | −0.69439 | 0.001809 |
| 207436_x_at | KIAA0894 | KIAA0894 protein | −0.69497 | 0.007507 |
| 214222_at | DNAH7 | dynein, axonemal, heavy polypeptide 7 | −0.69648 | 0.007442 |
| 203997_at | PTPN3 | protein tyrosine phosphatase, non-receptor type 3 | −0.69954 | 0.004439 |
| 219083_at | SHQ1 | SHQ1 homolog (S. cerevisiae) | −0.70178 | 0.009688 |
| 201099_at | USP9X | ubiquitin specific peptidase 9, X-linked (fat facets-like, | −0.70233 | 0.009047 |
| | | Drosophila) | | |
| 209111_at | RNF5 | ring finger protein 5 | −0.70262 | 0.009362 |
| 220355_s_at | PB1 | polybromo 1 | −0.70314 | 0.005192 |
| 214198_s_at | DGCR2 | DiGeorge syndrome critical region gene 2 | −0.70371 | 0.006994 |
| 212955_s_at | POLR2I | polymerase (RNA) II (DNA directed) polypeptide I, 14.5 kDa | −0.70648 | 0.000567 |
| 215416_s_at | STOML2 | stomatin (EPB72)-like 2 | −0.70672 | 0.002689 |
| 214527_s_at | PQBP1 | polyglutamine binding protein 1 | −0.70677 | 0.003258 |
| 211704_s_at | SPIN2 /// SPIN-2 | spindlin family, member 2 /// spindlin family, member 2 /// | −0.70689 | 0.005422 |
| | | spindlin-like protein 2 /// spindlin-like protein 2 | | |
| 205436_s_at | H2AFX | H2A histone family, member X | −0.70734 | 0.004135 |
| 211657_at | CEACAM6 | carcinoembryonic antigen-related cell adhesion molecule 6 (non- | −0.70765 | 0.000182 |
| | | specific cross reacting antigen) /// carcinoembryonic antigen- | | |
| | | related cell adhesion molecule 6 (non-specific cross reacting | | |
| | | antigen) | | |
| 37425_g_at | CCHCR1 | coiled-coil alpha-helical rod protein 1 | −0.71122 | 0.002885 |
| 209150_s_at | TM9SF1 | transmembrane 9 superfamily member 1 | −0.71246 | 0.006477 |
| 37079_at | YDD19 | YDD19 protein | −0.71419 | 3.74E−05 |
| 218903_s_at | MGC2731 | hypothetical protein MGC2731 | −0.71584 | 0.003537 |
| 200093_s_at | HINT1 | histidine triad nucleotide binding protein 1 /// histidine triad | −0.71733 | 0.003056 |
| | | nucleotide binding protein 1 | | |
| 201701_s_at | PGRMC2 | progesterone receptor membrane component 2 | −0.7189 | 0.006357 |
| 213657_s_at | DKFZp547K1113 | Hypothetical protein DKFZp547K1113 | −0.71936 | 0.008774 |
| 218101_s_at | NDUFC2 | NADH dehydrogenase (ubiquinone) 1, subcomplex unknown, 2, | −0.72187 | 0.002398 |
| | | 14.5 kDa | | |
| 50221_at | TFEB | transcription factor EB | −0.72278 | 0.00618 |
| 212716_s_at | EIF3S12 | eukaryotic translation initiation factor 3, subunit 12 | −0.72348 | 0.002345 |
| 201864_at | GDI1 | GDP dissociation inhibitor 1 | −0.72449 | 0.001835 |
| 37590_g_at | DKFZp547K1113 | Hypothetical protein DKFZp547K1113 | −0.72792 | 0.007371 |
| 210947_s_at | MSH3 | mutS homolog 3 (E. coli) | −0.72828 | 0.003692 |
| 203339_at | SLC25A12 | solute carrier family 25 (mitochondrial carrier, Aralar), member | −0.7308 | 0.008908 |
| | | 12 | | |
| 219022_at | FLJ12448 | hypothetical protein FLJ12448 | −0.73413 | 0.0048 |
| 201684_s_at | C14orf92 | chromosome 14 open reading frame 92 | −0.73479 | 0.007543 |
| 219350_s_at | DIABLO | diablo homolog (Drosophila) | −0.73687 | 0.00117 |
| 203506_s_at | MED12 | mediator of RNA polymerase II transcription, subunit 12 homolog | −0.73746 | 0.004262 |
| | | (yeast) | | |
| 208878_s_at | PAK2 | p21 (CDKN1A)-activated kinase 2 | −0.73827 | 0.004801 |
| 217831_s_at | NSFL1C | NSFL1 (p97) cofactor (p47) | −0.73915 | 0.00989 |
| 218476_at | POMT1 | protein-O-mannosyltransferase 1 | −0.73938 | 0.000183 |
| 222312_s_at | — | CDNA clone IMAGE: 6186815 | −0.73944 | 0.000353 |
| 200009_at | GDI2 | GDP dissociation inhibitor 2 /// GDP dissociation inhibitor 2 | −0.73962 | 0.001286 |
| 202732_at | PKIG | protein kinase (cAMP-dependent, catalytic) inhibitor gamma | −0.73979 | 0.002375 |
| 219218_at | KIAA1447 | KIAA1447 protein | −0.74076 | 0.004768 |
| 39729_at | PRDX2 | peroxiredoxin 2 | −0.74077 | 0.006552 |
| 213282_at | SATL1 | Spermidine/spermine N1-acetyl transferase-like 1 | −0.74188 | 0.004977 |
| 214964_at | KIAA1856 | KIAA1856 protein | −0.74498 | 0.000132 |
| 200893_at | SFRS10 | splicing factor, arginine/serine-rich 10 (transformer 2 homolog, | −0.74677 | 0.007868 |
| | | Drosophila) | | |
| 201628_s_at | RRAGA | Ras-related GTP binding A | −0.7476 | 0.009628 |
| 211759_x_at | CKAP1 | cytoskeleton associated protein 1 /// cytoskeleton associated | −0.74911 | 0.0055 |
| | | protein 1 | | |
| 204449_at | PDCL | phosducin-like | −0.74983 | 0.00022 |
| 208787_at | MRPL3 | mitochondrial ribosomal protein L3 | −0.75252 | 0.004371 |
| 222251_s_at | GMEB2 | glucocorticoid modulatory element binding protein 2 | −0.75259 | 0.001714 |
| 218003_s_at | FKBP3 | FK506 binding protein 3, 25 kDa | −0.75263 | 0.00434 |
| 214123_s_at | C4orf10 | chromosome 4 open reading frame 10 | −0.75269 | 0.009828 |
| 201065_s_at | GTF2I /// | general transcription factor II, i /// general transcription factor II, i, | −0.75366 | 0.00232 |
| | GTF2IP1 | pseudogene 1 | | |
| 36554_at | ASMTL | acetylserotonin O-methyltransferase-like | −0.75397 | 0.00268 |
| 203957_at | E2F6 | E2F transcription factor 6 | −0.75421 | 0.003532 |
| 201757_at | NDUFS5 | NADH dehydrogenase (ubiquinone) Fe—S protein 5, 15 kDa | −0.75444 | 0.001836 |
| | | (NADH-coenzyme Q reductase) | | |
| 201536_at | DUSP3 | dual specificity phosphatase 3 (vaccinia virus phosphatase VH1- | −0.7556 | 0.009559 |
| | | related) | | |
| 202744_at | SLC20A2 | solute carrier family 20 (phosphate transporter), member 2 | −0.75578 | 0.004961 |
| 211572_s_at | SLC23A2 | solute carrier family 23 (nucleobase transporters), member 2 | −0.75627 | 0.009553 |
| 201144_s_at | EIF2S1 | eukaryotic translation initiation factor 2, subunit 1 alpha, 35 kDa | −0.75696 | 0.006126 |
| 215039_at | LOC339524 | Hypothetical protein LOC339524 | −0.75704 | 0.006201 |
| 214714_at | ZNF394 | zinc finger protein 394 | −0.75718 | 0.002179 |
| 214383_x_at | KLHDC3 | kelch domain containing 3 | −0.75782 | 0.002199 |
| 216088_s_at | PSMA7 | proteasome (prosome, macropain) subunit, alpha type, 7 | −0.76088 | 0.002077 |
| 212380_at | KIAA0082 | KIAA0082 | −0.76138 | 0.000214 |
| 204244_s_at | ASK | activator of S phase kinase | −0.76263 | 5.14E−05 |
| 202000_at | NDUFA6 | NADH dehydrogenase (ubiquinone) 1 alpha subcomplex, 6, | −0.76272 | 0.004171 |
| | | 14 kDa | | |
| 205690_s_at | G10 | G10 protein | −0.76292 | 0.000527 |
| 211375_s_at | ILF3 | interleukin enhancer binding factor 3, 90 kDa | −0.76309 | 0.009228 |
| 221276_s_at | SYNC1 | syncoilin, intermediate filament 1 /// syncoilin, intermediate | −0.76526 | 0.002074 |
| | | filament 1 | | |
| 216387_x_at | LOC390411 | similar to nucleophosmin 1; nucleolar phosphoprotein B23; | −0.76553 | 0.001159 |
| | | numatrin; nucleophosmin/nucleoplasmin family, member 1 | | |
| 212891_s_at | GADD45GIP1 | growth arrest and DNA-damage-inducible, gamma interacting | −0.76639 | 0.000413 |
| | | protein 1 | | |
| 200765_x_at | CTNNA1 | catenin (cadherin-associated protein), alpha 1, 102 kDa | −0.76763 | 0.003259 |
| 208726_s_at | EIF2S2 | eukaryotic translation initiation factor 2, subunit 2 beta, 38 kDa | −0.77034 | 0.003614 |
| 201966_at | NDUFS2 | NADH dehydrogenase (ubiquinone) Fe—S protein 2, 49 kDa | −0.7704 | 0.000292 |
| | | (NADH-coenzyme Q reductase) | | |
| 208776_at | PSMD11 | proteasome (prosome, macropain) 26S subunit, non-ATPase, 11 | −0.7716 | 0.007918 |
| 219261_at | C7orf26 | chromosome 7 open reading frame 26 | −0.77182 | 0.008507 |
| 202123_s_at | ABL1 | v-abl Abelson murine leukemia viral oncogene homolog 1 | −0.77298 | 1.65E−05 |
| 209421_at | MSH2 | mutS homolog 2, colon cancer, nonpolyposis type 1 (E. coli) | −0.77673 | 0.008368 |
| 202882_x_at | NOL7 | nucleolar protein 7, 27 kDa | −0.77673 | 0.000681 |
| 209073_s_at | NUMB | numb homolog (Drosophila) | −0.77713 | 0.004734 |
| 219178_at | QTRTD1 | queuine tRNA-ribosyltransferase domain containing 1 | −0.77732 | 0.002286 |
| 205371_s_at | DBT | dihydrolipoamide branched chain transacylase E2 | −0.77873 | 6.59E−05 |
| 202110_at | COX7B | cytochrome c oxidase subunit VIIb | −0.77934 | 0.00334 |
| 212566_at | MAP4 | microtubule-associated protein 4 | −0.78273 | 0.007288 |
| 209249_s_at | GHITM | growth hormone inducible transmembrane protein | −0.78291 | 0.006153 |
| 219902_at | BHMT2 | betaine-homocysteine methyltransferase 2 | −0.78359 | 0.00835 |
| 203694_s_at | DHX16 | DEAH (Asp-Glu-Ala-His) box polypeptide 16 | −0.7836 | 0.003059 |
| 218123_at | C21orf59 | chromosome 21 open reading frame 59 | −0.78465 | 0.006791 |
| 201481_s_at | PYGB | phosphorylase, glycogen; brain | −0.78614 | 0.001801 |
| 215146_s_at | KIAA1043 | KIAA1043 protein | −0.78786 | 0.002939 |
| 202055_at | KPNA1 | Karyopherin alpha 1 (importin alpha 5) | −0.78996 | 0.00943 |
| 200639_s_at | YWHAZ | tyrosine 3-monooxygenase/tryptophan 5-monooxygenase | −0.79009 | 0.000534 |
| | | activation protein, zeta polypeptide | | |
| 203135_at | TBP | TATA box binding protein | −0.79059 | 0.009732 |
| 213317_at | CLIC5 | Chloride intracellular channel 5 | −0.79072 | 0.001612 |
| 203493_s_at | PIG8 | translokin | −0.79444 | 0.001565 |
| 221808_at | RAB9A | RAB9A, member RAS oncogene family | −0.79472 | 0.00961 |
| 219234_x_at | SCRN3 | secernin 3 | −0.79489 | 0.008368 |
| 203779_s_at | EVA1 | epithelial V-like antigen 1 | −0.79538 | 0.00324 |
| 205795_at | NRXN3 | neurexin 3 | −0.7969 | 0.002457 |
| 200775_s_at | HNRPK | heterogeneous nuclear ribonucleoprotein K | −0.79696 | 0.007965 |
| 201821_s_at | TIMM17A | translocase of inner mitochondrial membrane 17 homolog A | −0.79714 | 0.006797 |
| | | (yeast) | | |
| 207855_s_at | CLCC1 | chloride channel CLIC-like 1 | −0.79717 | 0.007537 |
| 204853_at | ORC2L | origin recognition complex, subunit 2-like (yeast) | −0.80051 | 0.009936 |
| 218336_at | PFDN2 | prefoldin 2 | −0.80169 | 0.004881 |
| 218360_at | RAB22A | RAB22A, member RAS oncogene family | −0.80204 | 0.008183 |
| 220647_s_at | CHCHD8 | coiled-coil-helix-coiled-coil-helix domain containing 8 | −0.8027 | 0.001123 |
| 203965_at | USP20 | ubiquitin specific peptidase 20 | −0.80294 | 0.004216 |
| 202233_s_at | UQCRH | ubiquinol-cytochrome c reductase hinge protein | −0.80333 | 0.001309 |
| 217800_s_at | NDFIP1 | Nedd4 family interacting protein 1 | −0.8037 | 0.006395 |
| 202614_at | SLC30A9 | solute carrier family 30 (zinc transporter), member 9 | −0.8047 | 0.005144 |
| 212393_at | SBF1 | SET binding factor 1 | −0.80587 | 0.008087 |
| 217921_at | MAN1A2 | Mannosidase, alpha, class 1A, member 2 | −0.80689 | 0.000406 |
| 208919_s_at | NADK | NAD kinase | −0.80699 | 0.000818 |
| 201568_at | QP-C | low molecular mass ubiquinone-binding protein (9.5 kD) | −0.80731 | 0.003485 |
| 212911_at | DNAJC16 | DnaJ (Hsp40) homolog, subfamily C, member 16 | −0.80792 | 0.003997 |
| 208264_s_at | EIF3S1 | eukaryotic translation initiation factor 3, subunit 1 alpha, 35 kDa | −0.80976 | 0.007801 |
| 218497_s_at | RNASEH1 | ribonuclease H1 | −0.80989 | 0.005866 |
| 201322_at | ATP5B | ATP synthase, H+ transporting, mitochondrial F1 complex, beta | −0.81106 | 0.000488 |
| | | polypeptide | | |
| 221595_at | DKFZP564O0523 | hypothetical protein DKFZp564O0523 | −0.8116 | 8.13E−06 |
| 202467_s_at | COPS2 | COP9 constitutive photomorphogenic homolog subunit 2 | −0.81229 | 0.006128 |
| | | (Arabidopsis) | | |
| 211871_x_at | GNB5 | guanine nucleotide binding protein (G protein), beta 5 | −0.81463 | 0.00138 |
| 208802_at | SRP72 | signal recognition particle 72 kDa | −0.81663 | 0.001571 |
| 207983_s_at | STAG2 | stromal antigen 2 | −0.81844 | 0.006387 |
| 208799_at | PSMB5 | proteasome (prosome, macropain) subunit, beta type, 5 | −0.8215 | 0.004553 |
| 206571_s_at | MAP4K4 | mitogen-activated protein kinase kinase kinase kinase 4 | −0.82224 | 0.000198 |
| 203550_s_at | C1orf2 | chromosome 1 open reading frame 2 | −0.8228 | 0.000237 |
| 203623_at | PLXNA3 | plexin A3 | −0.82341 | 0.004347 |
| 204120_s_at | ADK | adenosine kinase | −0.82518 | 2.08E−05 |
| 204605_at | CGRRF1 | cell growth regulator with ring finger domain 1 | −0.82605 | 0.002407 |
| 208938_at | PRCC | papillary renal cell carcinoma (translocation-associated) | −0.82699 | 0.006265 |
| 204246_s_at | DCTN3 | dynactin 3 (p22) | −0.82753 | 0.001424 |
| 202611_s_at | CRSP2 | cofactor required for Sp1 transcriptional activation, subunit 2, | −0.82774 | 0.005406 |
| | | 150 kDa | | |
| 203943_at | KIF3B | kinesin family member 3B | −0.82904 | 0.003121 |
| 221139_s_at | CSAD | cysteine sulfinic acid decarboxylase | −0.82905 | 0.004226 |
| 201825_s_at | SCCPDH | saccharopine dehydrogenase (putative) | −0.83069 | 0.002017 |
| 208777_s_at | PSMD11 | proteasome (prosome, macropain) 26S subunit, non-ATPase, 11 | −0.83082 | 0.005915 |
| 210076_x_at | SERBP1 | SERPINE1 mRNA binding protein 1 | −0.83127 | 0.00128 |
| 210131_x_at | SDHC | succinate dehydrogenase complex, subunit C, integral | −0.83127 | 0.009409 |
| | | membrane protein, 15 kDa | | |
| 221857_s_at | TJAP1 | tight junction associated protein 1 (peripheral) | −0.83192 | 0.001396 |
| 216693_x_at | HDGFRP3 | hepatoma-derived growth factor, related protein 3 | −0.83218 | 0.009332 |
| 201973_s_at | C7orf28A | chromosome 7 open reading frame 28A | −0.83281 | 0.000431 |
| 216308_x_at | GRHPR | glyoxylate reductase/hydroxypyruvate reductase | −0.83331 | 0.00067 |
| 201124_at | ITGB5 | integrin, beta 5 | −0.8334 | 0.000927 |
| 205170_at | STAT2 | signal transducer and activator of transcription 2, 113 kDa | −0.83387 | 0.007919 |
| 202824_s_at | TCEB1 | transcription elongation factor B (SIII), polypeptide 1 (15 kDa, | −0.836 | 0.006587 |
| | | elongin C) | | |
| 219685_at | TMEM35 | transmembrane protein 35 | −0.83656 | 0.000411 |
| 213794_s_at | C14orf120 | chromosome 14 open reading frame 120 | −0.83689 | 4.43E−07 |
| 219226_at | CRK7 | CDC2-related protein kinase 7 | −0.83753 | 6.50E−05 |
| 215982_s_at | DOM3Z | dom-3 homolog Z (C. elegans) | −0.83808 | 2.48E−05 |
| 221741_s_at | YTHDF1 | YTH domain family, member 1 | −0.83814 | 9.99E−05 |
| 221821_s_at | FLJ20436 | hypothetical protein FLJ20436 | −0.83905 | 0.002198 |
| 201138_s_at | SSB | Sjogren syndrome antigen B (autoantigen La) | −0.84033 | 0.001222 |
| 214830_at | SLC38A6 | solute carrier family 38, member 6 | −0.8413 | 0.000189 |
| 221246_x_at | TNS1 | tensin 1 /// tensin 1 | −0.84138 | 0.000662 |
| 207941_s_at | RNPC2 | RNA-binding region (RNP1, RRM) containing 2 | −0.84168 | 0.005956 |
| 211316_x_at | CFLAR | CASP8 and FADD-like apoptosis regulator | −0.84169 | 0.001909 |
| 212864_at | CDS2 | CDP-diacylglycerol synthase (phosphatidate cytidylyltransferase) 2 | −0.84201 | 0.000675 |
| 204408_at | APEX2 | APEX nuclease (apurinic/apyrimidinic endonuclease) 2 | −0.84255 | 0.001288 |
| 203664_s_at | POLR2D | polymerase (RNA) II (DNA directed) polypeptide D | −0.84272 | 9.73E−05 |
| 209412_at | TMEM1 | transmembrane protein 1 | −0.84277 | 0.005232 |
| 200811_at | CIRBP | cold inducible RNA binding protein | −0.84277 | 0.000801 |
| 201013_s_at | PAICS | phosphoribosylaminoimidazole carboxylase, | −0.84408 | 0.005497 |
| | | phosphoribosylaminoimidazole succinocarboxamide synthetase | | |
| 209196_at | WDR46 | WD repeat domain 46 | −0.84428 | 0.005078 |
| 212048_s_at | YARS | tyrosyl-tRNA synthetase | −0.84458 | 0.000572 |
| 207555_s_at | TBXA2R | thromboxane A2 receptor | −0.84479 | 0.004461 |
| 201133_s_at | PJA2 | praja 2, RING-H2 motif containing | −0.84505 | 0.008199 |
| 212036_s_at | PNN | pinin, desmosome associated protein | −0.84515 | 0.001336 |
| 202791_s_at | SAPS2 | SAPS domain family, member 2 | −0.84605 | 0.007419 |
| 208289_s_at | EI24 | etoposide induced 2.4 mRNA | −0.84628 | 3.00E−05 |
| 212073_at | CSNK2A1 /// | casein kinase 2, alpha 1 polypeptide /// casein kinase 2, alpha 1 | −0.84672 | 0.003804 |
| | CSNK2A1P | polypeptide pseudogene | | |
| 201673_s_at | GYS1 | glycogen synthase 1 (muscle) | −0.84864 | 0.007973 |
| 204496_at | STRN3 | striatin, calmodulin binding protein 3 | −0.84902 | 0.004414 |
| 209455_at | FBXW11 | F-box and WD-40 domain protein 11 | −0.84965 | 0.004894 |
| 210658_s_at | GGA2 | golgi associated, gamma adaptin ear containing, ARF binding | −0.85038 | 0.007416 |
| | | protein 2 | | |
| 215093_at | NSDHL | NAD(P) dependent steroid dehydrogenase-like | −0.85127 | 9.01E−05 |
| 203006_at | INPP5A | inositol polyphosphate-5-phosphatase, 40 kDa | −0.85213 | 0.001768 |
| 201807_at | VPS26A | vacuolar protein sorting 26 homolog A (yeast) | −0.85244 | 6.93E−06 |
| 218008_at | FLJ10099 | hypothetical protein FLJ10099 | −0.85328 | 0.008515 |
| 213327_s_at | USP12 | ubiquitin specific peptidase 12 | −0.85504 | 0.000582 |
| 218443_s_at | DAZAP1 | DAZ associated protein 1 | −0.85625 | 0.000819 |
| 200668_s_at | UBE2D3 | ubiquitin-conjugating enzyme E2D 3 (UBC4/5 homolog, yeast) | −0.85733 | 0.002757 |
| 218374_s_at | C12orf4 | chromosome 12 open reading frame 4 | −0.85779 | 0.006888 |
| 201377_at | UBAP2L | ubiquitin associated protein 2-like | −0.8578 | 0.000326 |
| 202163_s_at | CNOT8 | CCR4-NOT transcription complex, subunit 8 | −0.85805 | 0.004949 |
| 213775_x_at | ZNF638 | zinc finger protein 638 | −0.85826 | 0.004958 |
| 208858_s_at | FAM62A | family with sequence similarity 62 (C2 domain containing), | −0.85872 | 0.002322 |
| | | member A | | |
| 218046_s_at | MRPS16 | mitochondrial ribosomal protein S16 | −0.86051 | 0.001044 |
| 212381_at | USP24 | ubiquitin specific peptidase 24 | −0.86145 | 0.001328 |
| 203340_s_at | SLC25A12 | solute carrier family 25 (mitochondrial carrier, Aralar), member | −0.86221 | 0.001985 |
| | | 12 | | |
| 222045_s_at | C20orf67 | chromosome 20 open reading frame 67 | −0.86255 | 4.13E−06 |
| 218572_at | CHMP4A | chromatin modifying protein 4A | −0.86276 | 0.006257 |
| 219390_at | FKBP14 | FK506 binding protein 14, 22 kDa | −0.86324 | 0.001176 |
| 200896_x_at | HDGF | hepatoma-derived growth factor (high-mobility group protein 1- | −0.86388 | 0.003929 |
| | | like) | | |
| 217786_at | SKB1 | SKB1 homolog (S. pombe) | −0.86411 | 0.000721 |
| 209861_s_at | METAP2 | methionyl aminopeptidase 2 | −0.86445 | 0.000954 |
| 206554_x_at | SETMAR | SET domain and mariner transposase fusion gene | −0.86475 | 0.00792 |
| 222250_s_at | C1orf73 | chromosome 1 open reading frame 73 | −0.86561 | 0.00025 |
| 219007_at | NUP43 | nucleoporin 43 kDa | −0.86661 | 0.00189 |
| 208789_at | PTRF | polymerase I and transcript release factor | −0.86812 | 0.000584 |
| 212780_at | SOS1 | son of sevenless homolog 1 (Drosophila) | −0.87025 | 0.008871 |
| 208336_s_at | GPSN2 | glycoprotein, synaptic 2 | −0.87241 | 0.007563 |
| 203781_at | MRPL33 | mitochondrial ribosomal protein L33 | −0.87244 | 8.62E−05 |
| 212138_at | SCC-112 | SCC-112 protein | −0.8729 | 0.007291 |
| 213786_at | TAX1BP1 | Tax1 (human T-cell leukemia virus type I) binding protein 1 | −0.87526 | 0.002834 |
| 219119_at | LSM8 | LSM8 homolog, U6 small nuclear RNA associated (S. cerevisiae) | −0.87625 | 0.004966 |
| 208672_s_at | SFRS3 | splicing factor, arginine/serine-rich 3 | −0.87638 | 0.001021 |
| 43427_at | ACACB | acetyl-Coenzyme A carboxylase beta | −0.87675 | 9.47E−05 |
| 202777_at | SHOC2 | soc-2 suppressor of clear homolog (C. elegans) | −0.87754 | 0.00084 |
| 201276_at | RAB5B | RAB5B, member RAS oncogene family | −0.8784 | 0.007545 |
| 218449_at | FLJ11200 | hypothetical protein FLJ11200 | −0.8788 | 0.005059 |
| 215696_s_at | KIAA0310 | KIAA0310 | −0.87908 | 0.007433 |
| 218026_at | HSPC009 | HSPC009 protein | −0.88067 | 0.006484 |
| 211475_s_at | BAG1 | BCL2-associated athanogene | −0.88127 | 0.00152 |
| 218791_s_at | C15orf29 | chromosome 15 open reading frame 29 | −0.88175 | 0.005425 |
| 213026_at | ATG12 | ATG12 autophagy related 12 homolog (S. cerevisiae) | −0.88307 | 0.008942 |
| 201253_s_at | CDIPT | CDP-diacylglycerol--inositol 3-phosphatidyltransferase | −0.88387 | 0.00736 |
| | | (phosphatidylinositol synthase) | | |
| 211703_s_at | TM2D1 | TM2 domain containing 1 /// TM2 domain containing 1 | −0.88396 | 0.00368 |
| 218583_s_at | DCUN1D1 | DCN1, defective in cullin neddylation 1, domain containing 1 | −0.88542 | 0.005154 |
| | | (S. cerevisiae) | | |
| 217398_x_at | GAPDH | glyceraldehyde-3-phosphate dehydrogenase | −0.88557 | 0.004769 |
| 217902_s_at | HERC2 | hect domain and RLD 2 | −0.88669 | 0.006504 |
| 215397_x_at | — | MRNA; cDNA DKFZp686P02129 (from clone DKFZp686P02129) | −0.88705 | 0.005422 |
| 210273_at | PCDH7 | BH-protocadherin (brain-heart) | −0.88718 | 0.007732 |
| 206200_s_at | ANXA11 | annexin A11 | −0.88784 | 0.000676 |
| 201052_s_at | PSMF1 | proteasome (prosome, macropain) inhibitor subunit 1 (PI31) | −0.88808 | 0.002153 |
| 213453_x_at | GAPDH | glyceraldehyde-3-phosphate dehydrogenase | −0.88826 | 0.004237 |
| 206992_s_at | ATP5S | ATP synthase, H+ transporting, mitochondrial F0 complex, | −0.88933 | 2.42E−05 |
| | | subunit s (factor B) | | |
| 217898_at | C15orf24 | chromosome 15 open reading frame 24 | −0.88935 | 0.003988 |
| 208420_x_at | SUPT6H | suppressor of Ty 6 homolog (S. cerevisiae) | −0.88989 | 0.002208 |
| 212403_at | UBE3B | ubiquitin protein ligase E3B | −0.89175 | 0.00104 |
| 201157_s_at | NMT1 | N-myristoyltransferase 1 | −0.89508 | 0.003883 |
| 212338_at | MYO1D | myosin ID | −0.897 | 0.000299 |
| 216129_at | ATP9A | ATPase, Class II, type 9A | −0.89784 | 0.004271 |
| 212430_at | RNPC1 | RNA-binding region (RNP1, RRM) containing 1 /// RNA-binding | −0.89832 | 0.002811 |
| | | region (RNP1, RRM) containing 1 | | |
| 203667_at | TBCA | tubulin-specific chaperone a | −0.90028 | 0.00024 |
| 212111_at | STX12 | syntaxin 12 | −0.90239 | 0.006297 |
| 212213_x_at | OPA1 | optic atrophy 1 (autosomal dominant) | −0.90347 | 0.008425 |
| 206374_at | DUSP8 | dual specificity phosphatase 8 | −0.90383 | 0.002229 |
| 218235_s_at | UTP11L | UTP11-like, U3 small nucleolar ribonucleoprotein, (yeast) | −0.90452 | 1.71E−05 |
| 212500_at | C10orf22 | chromosome 10 open reading frame 22 | −0.90478 | 0.002156 |
| 204832_s_at | BMPR1A | bone morphogenetic protein receptor, type IA | −0.90595 | 0.006325 |
| 205348_s_at | DNCI1 | dynein, cytoplasmic, intermediate polypeptide 1 | −0.90771 | 0.006166 |
| 205794_s_at | NOVA1 | neuro-oncological ventral antigen 1 | −0.9105 | 0.008534 |
| 208773_s_at | ANKHD1 /// | ankyrin repeat and KH domain containing 1 /// MASK-4E-BP3 | −0.91093 | 0.005225 |
| | MASK-BP3 | alternate reading frame gene | | |
| 212600_s_at | UQCRC2 | ubiquinol-cytochrome c reductase core protein II | −0.91101 | 0.003683 |
| 207785_s_at | RBPSUH | recombining binding protein suppressor of hairless (Drosophila) | −0.91106 | 0.005934 |
| 201832_s_at | VDP | vesicle docking protein p115 | −0.91145 | 0.000844 |
| 203605_at | SRP54 | signal recognition particle 54 kDa | −0.91249 | 0.001527 |
| 202521_at | CTCF | CCCTC-binding factor (zinc finger protein) | −0.91285 | 0.001918 |
| 208911_s_at | PDHB | pyruvate dehydrogenase (lipoamide) beta | −0.91295 | 0.000755 |
| 218670_at | PUS1 | pseudouridylate synthase 1 | −0.91299 | 0.000437 |
| 213012_at | NEDD4 | neural precursor cell expressed, developmentally down- | −0.91306 | 0.005894 |
| | | regulated 4 | | |
| 203165_s_at | SLC33A1 | solute carrier family 33 (acetyl-CoA transporter), member 1 | −0.91312 | 0.005462 |
| 209882_at | RIT1 | Ras-like without CAAX 1 | −0.91519 | 0.008734 |
| 200759_x_at | NFE2L1 | nuclear factor (erythroid-derived 2)-like 1 | −0.91555 | 0.001635 |
| 209780_at | PHTF2 | putative homeodomain transcription factor 2 | −0.91575 | 0.008746 |
| 200915_x_at | KTN1 /// PDIA6 | kinectin 1 (kinesin receptor) /// protein disulfide isomerase family | −0.91578 | 0.002091 |
| | | A, member 6 | | |
| 203230_at | DVL1 | dishevelled, dsh homolog 1 (Drosophila) | −0.91602 | 0.001548 |
| 208874_x_at | PPP2R4 | protein phosphatase 2A, regulatory subunit B′ (PR 53) | −0.91621 | 0.004867 |
| 208670_s_at | CRI1 | CREBBP/EP300 inhibitor 1 | −0.91762 | 0.002796 |
| 219805_at | FLJ22965 | hypothetical protein FLJ22965 | −0.91774 | 2.46E−05 |
| 216563_at | ANKRD12 | Ankyrin repeat domain 12 | −0.91808 | 0.002383 |
| 213034_at | KIAA0999 | KIAA0999 protein | −0.91834 | 0.001001 |
| 213027_at | SSA2 | TROVE domain family, member 2 | −0.91909 | 0.002879 |
| 202776_at | ERBP | estrogen receptor binding protein | −0.91971 | 3.60E−05 |
| 206015_s_at | FOXJ3 | forkhead box J3 | −0.91988 | 0.00879 |
| 208838_at | CAND1 | cullin-associated and neddylation-dissociated 1 | −0.92012 | 3.22E−05 |
| 202706_s_at | UMPS | uridine monophosphate synthetase (orotate phosphoribosyl | −0.92049 | 0.004614 |
| | | transferase and orotidine-5′-decarboxylase) | | |
| 201027_s_at | EIF5B | eukaryotic translation initiation factor 5B | −0.92103 | 0.002296 |
| 204984_at | GPC4 | glypican 4 | −0.92211 | 0.005849 |
| 213408_s_at | PIK4CA /// | phosphatidylinositol 4-kinase, catalytic, alpha polypeptide /// | −0.92308 | 0.001449 |
| | LOC220686 | hypothetical protein LOC220686 | | |
| 202347_s_at | HIP2 | huntingtin interacting protein 2 | −0.92361 | 0.001499 |
| 214170_x_at | FH | fumarate hydratase | −0.92479 | 9.50E−05 |
| 208780_x_at | VAPA | VAMP (vesicle-associated membrane protein)-associated protein | −0.92645 | 0.000648 |
| | | A, 33 kDa | | |
| 213021_at | GOSR1 | golgi SNAP receptor complex member 1 | −0.92673 | 0.002543 |
| 215722_s_at | SNRPA1 | small nuclear ribonucleoprotein polypeptide A′ | −0.92727 | 0.002327 |
| 209096_at | UBE2V2 | ubiquitin-conjugating enzyme E2 variant 2 | −0.92736 | 0.008304 |
| 212162_at | KIDINS220 | kinase D-interacting substance of 220 kDa | −0.92795 | 0.004971 |
| 205012_s_at | HAGH | hydroxyacylglutathione hydrolase | −0.92836 | 0.007196 |
| 200594_x_at | HNRPU | heterogeneous nuclear ribonucleoprotein U (scaffold attachment | −0.92861 | 6.67E−05 |
| | | factor A) | | |
| 203368_at | CRELD1 | cysteine-rich with EGF-like domains 1 | −0.93004 | 0.006363 |
| 208132_x_at | BAT2 | HLA-B associated transcript 2 | −0.9309 | 0.008491 |
| 55692_at | ELMO2 | engulfment and cell motility 2 (ced-12 homolog, C. elegans) | −0.93114 | 0.009216 |
| 212855_at | DCUN1D4 | DCN1, defective in cullin neddylation 1, domain containing 4 | −0.93212 | 0.000803 |
| | | (S. cerevisiae) | | |
| 212064_x_at | MAZ | MYC-associated zinc finger protein (purine-binding transcription | −0.93264 | 0.008194 |
| | | factor) | | |
| 202502_at | ACADM | acyl-Coenzyme A dehydrogenase, C-4 to C-12 straight chain | −0.9328 | 0.001303 |
| 202590_s_at | PDK2 | pyruvate dehydrogenase kinase, isoenzyme 2 | −0.93451 | 0.0003 |
| 219219_at | FLJ20512 | hypothetical protein FLJ20512 | −0.93495 | 0.006198 |
| 215460_x_at | BRD1 | bromodomain containing 1 | −0.93499 | 0.008519 |
| 201202_at | PCNA | proliferating cell nuclear antigen | −0.93499 | 0.002103 |
| 202299_s_at | HBXIP | hepatitis B virus x interacting protein | −0.93607 | 0.000487 |
| 201696_at | SFRS4 | splicing factor, arginine/serine-rich 4 | −0.93706 | 0.001347 |
| 209580_s_at | MBD4 | methyl-CpG binding domain protein 4 | −0.93852 | 0.006199 |
| 218174_s_at | C10orf57 | chromosome 10 open reading frame 57 | −0.93894 | 0.000781 |
| 208990_s_at | HNRPH3 | heterogeneous nuclear ribonucleoprotein H3 (2H9) | −0.93905 | 0.004121 |
| 209741_x_at | ZNF291 | zinc finger protein 291 | −0.94051 | 0.007117 |
| 207098_s_at | MFN1 | mitofusin 1 | −0.94058 | 0.003924 |
| 217970_s_at | CNOT6 | CCR4-NOT transcription complex, subunit 6 | −0.94155 | 0.009776 |
| 201057_s_at | GOLGB1 | golgi autoantigen, golgin subfamily b, macrogolgin (with | −0.94299 | 0.000229 |
| | | transmembrane signal), 1 | | |
| 218389_s_at | APH1A | anterior pharynx defective 1 homolog A (C. elegans) | −0.94301 | 0.000602 |
| 201769_at | ENTH | enthoprotin | −0.94322 | 0.002475 |
| 218432_at | FBXO3 | F-box protein 3 | −0.94331 | 0.009769 |
| 216105_x_at | PPP2R4 | protein phosphatase 2A, regulatory subunit B′ (PR 53) | −0.94451 | 0.001092 |
| 203176_s_at | TFAM | transcription factor A, mitochondrial | −0.94492 | 0.007361 |
| 221540_x_at | GTF2H2 | general transcription factor IIH, polypeptide 2, 44 kDa | −0.94562 | 1.16E−05 |
| 212069_s_at | KIAA0515 | KIAA0515 | −0.94765 | 0.005419 |
| 214484_s_at | OPRS1 | opioid receptor, sigma 1 | −0.94795 | 0.007569 |
| 200657_at | SLC25A5 | solute carrier family 25 (mitochondrial carrier; adenine nucleotide | −0.94896 | 0.008475 |
| | | translocator), member 5 | | |
| 218568_at | MULK | multiple substrate lipid kinase | −0.94904 | 0.003943 |
| 210216_x_at | RAD1 | RAD1 homolog (S. pombe) | −0.94946 | 8.08E−06 |
| 208642_s_at | XRCC5 | X-ray repair complementing defective repair in Chinese hamster | −0.94998 | 0.000694 |
| | | cells 5 (double-strand-break rejoining; Ku autoantigen, 80 kDa) | | |
| 212010_s_at | H41 | hypothetical protein H41 | −0.95179 | 0.003062 |
| 60815_at | MGC13098 | hypothetical protein MGC13098 | −0.95269 | 0.003992 |
| 200627_at | PTGES3 | prostaglandin E synthase 3 (cytosolic) | −0.95292 | 2.57E−05 |
| 218766_s_at | WARS2 | tryptophanyl tRNA synthetase 2 (mitochondrial) | −0.95304 | 1.52E−05 |
| 200974_at | ACTA2 | actin, alpha 2, smooth muscle, aorta | −0.95308 | 0.004215 |
| 201879_at | ARIH1 | ariadne homolog, ubiquitin-conjugating enzyme E2 binding | −0.95377 | 0.000505 |
| | | protein, 1 (Drosophila) | | |
| 203856_at | VRK1 | vaccinia related kinase 1 | −0.95396 | 0.008204 |
| 204345_at | COL16A1 | collagen, type XVI, alpha 1 | −0.95413 | 0.000963 |
| 206142_at | ZNF135 | zinc finger protein 135 (clone pHZ-17) | −0.95421 | 0.007522 |
| 200950_at | ARPC1A | actin related protein 2/3 complex, subunit 1A, 41 kDa | −0.95597 | 0.003059 |
| 202730_s_at | PDCD4 | programmed cell death 4 (neoplastic transformation inhibitor) | −0.95616 | 0.001514 |
| 218461_at | ATPBD1C | ATP binding domain 1 family, member C | −0.95684 | 0.000338 |
| 212211_at | ANKRD17 | ankyrin repeat domain 17 | −0.95747 | 0.006088 |
| 207508_at | ATP5G3 | ATP synthase, H+ transporting, mitochondrial F0 complex, | −0.95819 | 2.46E−05 |
| | | subunit c (subunit 9) isoform 3 | | |
| 218200_s_at | NDUFB2 | NADH dehydrogenase (ubiquinone) 1 beta subcomplex, 2, 8 kDa | −0.95829 | 0.008313 |
| 202867_s_at | DNAJB12 | DnaJ (Hsp40) homolog, subfamily B, member 12 | −0.95835 | 0.000851 |
| 222230_s_at | ACTR10 | actin-related protein 10 homolog (S. cerevisiae) | −0.95849 | 0.000176 |
| 216180_s_at | SYNJ2 | synaptojanin 2 | −0.96152 | 0.00817 |
| 217465_at | — | — | −0.96231 | 0.000449 |
| 204062_s_at | ULK2 | unc-51-like kinase 2 (C. elegans) | −0.96306 | 0.00036 |
| 218716_x_at | MTO1 | mitochondrial translation optimization 1 homolog (S. cerevisiae) | −0.96617 | 1.86E−05 |
| 205961_s_at | PSIP1 | PC4 and SFRS1 interacting protein 1 | −0.9673 | 0.001204 |
| 213478_at | KIAA1026 | kazrin | −0.97139 | 0.00033 |
| 201947_s_at | CCT2 | chaperonin containing TCP1, subunit 2 (beta) | −0.97198 | 0.001177 |
| 205738_s_at | FABP3 | fatty acid binding protein 3, muscle and heart (mammary-derived | −0.97255 | 0.009082 |
| | | growth inhibitor) | | |
| 212421_at | C22orf9 | chromosome 22 open reading frame 9 | −0.97269 | 0.000217 |
| 202328_s_at | PKD1 | polycystic kidney disease 1 (autosomal dominant) | −0.97294 | 0.003175 |
| 203369_x_at | PDLIM7 | PDZ and LIM domain 7 (enigma) | −0.97393 | 0.008188 |
| 214526_x_at | PMS2L1 | postmeiotic segregation increased 2-like 1 | −0.97448 | 0.002317 |
| 210097_s_at | NOL7 | nucleolar protein 7, 27 kDa | −0.97506 | 6.37E−05 |
| 202130_at | RIOK3 | RIO kinase 3 (yeast) /// RIO kinase 3 (yeast) | −0.97622 | 0.000324 |
| 200658_s_at | PHB | prohibitin | −0.97648 | 0.000448 |
| 200011_s_at | ARF3 | ADP-ribosylation factor 3 /// ADP-ribosylation factor 3 | −0.97665 | 3.54E−06 |
| 212199_at | MRFAP1L1 | Morf4 family associated protein 1-like 1 | −0.97755 | 0.005815 |
| 212904_at | LRRC47 | leucine rich repeat containing 47 | −0.9781 | 1.87E−05 |
| 221486_at | ENSA | endosulfine alpha | −0.97843 | 0.000278 |
| 208114_s_at | ISG20L2 | interferon stimulated exonuclease gene 20 kDa-like 2 /// | −0.97981 | 0.009832 |
| | | interferon stimulated exonuclease gene 20 kDa-like 2 | | |
| 221599_at | PTD015 | PTD015 protein | −0.98026 | 0.007796 |
| 212967_x_at | NAP1L1 | nucleosome assembly protein 1-like 1 | −0.98031 | 0.005454 |
| 202868_s_at | POP4 | processing of precursor 4, ribonuclease P/MRP subunit (S. cerevisiae) | −0.98037 | 0.000248 |
| 216689_x_at | ARHGAP1 | Rho GTPase activating protein 1 | −0.98106 | 0.003753 |
| 215227_x_at | ACP1 | acid phosphatase 1, soluble | −0.98177 | 0.005046 |
| 210707_x_at | PMS2L11 | postmeiotic segregation increased 2-like 11 | −0.98247 | 0.009867 |
| 212436_at | TRIM33 | tripartite motif-containing 33 | −0.98263 | 0.001538 |
| 209393_s_at | EIF4E2 | eukaryotic translation initiation factor 4E member 2 | −0.98508 | 0.001482 |
| 207776_s_at | CACNB2 | calcium channel, voltage-dependent, beta 2 subunit | −0.98682 | 0.003331 |
| 218239_s_at | GTPBP4 | GTP binding protein 4 | −0.9873 | 0.000605 |
| 221517_s_at | CRSP6 | cofactor required for Sp1 transcriptional activation, subunit 6, | −0.988 | 0.009335 |
| | | 77 kDa | | |
| 200793_s_at | ACO2 | aconitase 2, mitochondrial | −0.98921 | 0.001989 |
| 203869_at | USP46 | ubiquitin specific peptidase 46 | −0.99036 | 0.001197 |
| 201323_at | EBNA1BP2 | EBNA1 binding protein 2 | −0.99066 | 7.37E−05 |
| 218425_at | TRIAD3 | TRIAD3 protein | −0.99164 | 0.004899 |
| 204171_at | RPS6KB1 | ribosomal protein S6 kinase, 70 kDa, polypeptide 1 | −0.99214 | 0.0021 |
| 207628_s_at | WBSCR22 | Williams Beuren syndrome chromosome region 22 | −0.99457 | 0.001442 |
| 212347_x_at | MXD4 | MAX dimerization protein 4 | −0.99481 | 0.003251 |
| 220985_s_at | RNF170 | ring finger protein 170 /// ring finger protein 170 | −0.99481 | 0.004357 |
| 214071_at | MPPE1 | Metallophosphoesterase 1 | −0.99606 | 0.006101 |
| 201193_at | IDH1 | isocitrate dehydrogenase 1 (NADP+), soluble | −0.99696 | 0.00888 |
| 211025_x_at | COX5B | cytochrome c oxidase subunit Vb /// cytochrome c oxidase | −0.99767 | 0.000509 |
| | | subunit Vb | | |
| 200726_at | PPP1CC | protein phosphatase 1, catalytic subunit, gamma isoform | −0.99846 | 0.000448 |
| 213911_s_at | H2AFZ | H2A histone family, member Z | −1.00021 | 0.001011 |
| 201690_s_at | TPD52 | tumor protein D52 | −1.00068 | 0.001055 |
| 202164_s_at | CNOT8 | CCR4-NOT transcription complex, subunit 8 | −1.0013 | 0.002951 |
| 213761_at | MDM1 | Mdm4, transformed 3T3 cell double minute 1, p53 binding | −1.0017 | 0.007083 |
| | | protein (mouse) | | |
| 202001_s_at | NDUFA6 | NADH dehydrogenase (ubiquinone) 1 alpha subcomplex, 6, | −1.00365 | 0.002535 |
| | | 14 kDa | | |
| 212815_at | ASCC3 | activating signal cointegrator 1 complex subunit 3 | −1.00365 | 0.007719 |
| 202560_s_at | C1orf77 | chromosome 1 open reading frame 77 | −1.00491 | 0.006684 |
| 213224_s_at | LOC92482 | hypothetical protein LOC92482 | −1.00649 | 0.009743 |
| 219241_x_at | SSH3 | slingshot homolog 3 (Drosophila) | −1.00679 | 0.003077 |
| 221771_s_at | HSMPP8 | M-phase phosphoprotein, mpp8 | −1.00685 | 0.002191 |
| 215269_at | TMEM1 | transmembrane protein 1 | −1.00692 | 0.002319 |
| 219289_at | FLJ20718 | hypothetical protein FLJ20718 | −1.00725 | 0.000266 |
| 201804_x_at | CKAP1 | cytoskeleton associated protein 1 | −1.00754 | 2.95E−05 |
| 216976_s_at | RYK | RYK receptor-like tyrosine kinase | −1.00783 | 0.009465 |
| 205139_s_at | UST | uronyl-2-sulfotransferase | −1.00905 | 0.003928 |
| 221656_s_at | ARHGEF10L | Rho guanine nucleotide exchange factor (GEF) 10-like | −1.00959 | 0.000121 |
| 214121_x_at | PDLIM7 | PDZ and LIM domain 7 (enigma) | −1.01035 | 0.001663 |
| 200993_at | IPO7 | importin 7 | −1.0107 | 0.008706 |
| 202300_at | HBXIP | hepatitis B virus x interacting protein | −1.01179 | 4.71E−07 |
| 208643_s_at | XRCC5 | X-ray repair complementing defective repair in Chinese hamster | −1.01183 | 0.000305 |
| | | cells 5 (double-strand-break rejoining; Ku autoantigen, 80 kDa) | | |
| 201932_at | LRRC41 | leucine rich repeat containing 41 | −1.01255 | 0.001102 |
| 200609_s_at | WDR1 | WD repeat domain 1 | −1.0134 | 1.30E−07 |
| 212459_x_at | SUCLG2 | succinate-CoA ligase, GDP-forming, beta subunit | −1.0144 | 0.003795 |
| 218538_s_at | MRS2L | MRS2-like, magnesium homeostasis factor (S. cerevisiae) | −1.01529 | 0.007386 |
| 209333_at | ULK1 | unc-51-like kinase 1 (C. elegans) | −1.01582 | 0.000666 |
| 218518_at | C5orf5 | chromosome 5 open reading frame 5 | −1.01697 | 0.000881 |
| 204066_s_at | CENTG2 | centaurin, gamma 2 | −1.01741 | 0.006665 |
| 218116_at | C9orf78 | chromosome 9 open reading frame 78 /// chromosome 9 open | −1.01761 | 0.001146 |
| | | reading frame 78 | | |
| 203043_at | ZBED1 | zinc finger, BED-type containing 1 | −1.0179 | 0.009202 |
| 212359_s_at | KIAA0913 | KIAA0913 | −1.01848 | 0.002293 |
| 212652_s_at | SNX4 | sorting nexin 4 | −1.01866 | 0.004242 |
| 202591_s_at | SSBP1 | single-stranded DNA binding protein 1 | −1.01882 | 5.25E−05 |
| 209222_s_at | OSBPL2 | oxysterol binding protein-like 2 | −1.02044 | 1.02E−05 |
| 218197_s_at | OXR1 | oxidation resistance 1 | −1.02052 | 0.004987 |
| 212137_at | LARP1 | La ribonucleoprotein domain family, member 1 | −1.02133 | 7.94E−05 |
| 213090_s_at | TAF4 | TAF4 RNA polymerase II, TATA box binding protein (TBP)- | −1.02164 | 0.001047 |
| | | associated factor, 135 kDa | | |
| 221506_s_at | TNPO2 | transportin 2 (importin 3, karyopherin beta 2b) | −1.02281 | 0.002465 |
| 217956_s_at | MASA | E-1 enzyme | −1.02399 | 0.00121 |
| 217717_s_at | YWHAB | tyrosine 3-monooxygenase/tryptophan 5-monooxygenase | −1.02469 | 0.000672 |
| | | activation protein, beta polypeptide | | |
| 202316_x_at | UBE4B | ubiquitination factor E4B (UFD2 homolog, yeast) | −1.02486 | 0.00573 |
| 210685_s_at | UBE4B | ubiquitination factor E4B (UFD2 homolog, yeast) | −1.02524 | 0.001495 |
| 201630_s_at | ACP1 | acid phosphatase 1, soluble | −1.02561 | 0.000117 |
| 206284_x_at | CLTB | clathrin, light polypeptide (Lcb) | −1.02591 | 9.99E−05 |
| 200640_at | YWHAZ | tyrosine 3-monooxygenase/tryptophan 5-monooxygenase | −1.02604 | 0.000169 |
| | | activation protein, zeta polypeptide | | |
| 205995_x_at | IQCB1 | IQ motif containing B1 | −1.02804 | 0.000168 |
| 209338_at | TFCP2 | transcription factor CP2 | −1.0287 | 0.000924 |
| 210896_s_at | ASPH | aspartate beta-hydroxylase | −1.03002 | 8.36E−06 |
| 216194_s_at | CKAP1 | cytoskeleton associated protein 1 | −1.03074 | 0.000366 |
| 201706_s_at | PEX19 | peroxisomal biogenesis factor 19 | −1.03125 | 0.000137 |
| 203745_at | HCCS | holocytochrome c synthase (cytochrome c heme-lyase) | −1.03179 | 1.32E−05 |
| 201200_at | CREG1 | cellular repressor of E1A-stimulated genes 1 | −1.03453 | 0.002571 |
| 208875_s_at | PAK2 | p21 (CDKN1A)-activated kinase 2 | −1.03578 | 0.006745 |
| 202470_s_at | CPSF6 | cleavage and polyadenylation specific factor 6, 68 kDa | −1.03587 | 0.001503 |
| 218024_at | BRP44L | brain protein 44-like | −1.03651 | 7.54E−06 |
| 218866_s_at | POLR3K | polymerase (RNA) III (DNA directed) polypeptide K, 12.3 kDa | −1.03657 | 0.006995 |
| 211543_s_at | GRK6 | G protein-coupled receptor kinase 6 | −1.0366 | 0.000252 |
| 200870_at | STRAP | serine/threonine kinase receptor associated protein | −1.03712 | 0.003591 |
| 218434_s_at | AACS | acetoacetyl-CoA synthetase | −1.03766 | 0.000343 |
| 211337_s_at | 76P | gamma tubulin ring complex protein (76p gene) | −1.03789 | 0.001882 |
| 200758_s_at | NFE2L1 | nuclear factor (erythroid-derived 2)-like 1 | −1.03831 | 0.004021 |
| 217724_at | SERBP1 | SERPINE1 mRNA binding protein 1 | −1.04015 | 9.63E−05 |
| 202565_s_at | SVIL | supervillin | −1.04073 | 0.003733 |
| 221904_at | MGC21688 | hypothetical protein MGC21688 | −1.04076 | 0.002084 |
| 202325_s_at | ATP5J | ATP synthase, H+ transporting, mitochondrial F0 complex, | −1.04185 | 1.35E−05 |
| | | subunit F6 | | |
| 220741_s_at | PPA2 | pyrophosphatase (inorganic) 2 | −1.04358 | 0.000405 |
| 215023_s_at | PEX1 | peroxisome biogenesis factor 1 | −1.04364 | 0.004577 |
| 201018_at | EIF1AX | eukaryotic translation initiation factor 1A, X-linked | −1.04409 | 0.007807 |
| 218616_at | PHF22 | PHD finger protein 22 | −1.04424 | 0.005224 |
| 208909_at | UQCRFS1 | ubiquinol-cytochrome c reductase, Rieske iron-sulfur polypeptide 1 | −1.04471 | 4.41E−05 |
| 221193_s_at | ZCCHC10 | zinc finger, CCHC domain containing 10 | −1.04477 | 0.003533 |
| 219779_at | ZFHX4 | zinc finger homeodomain 4 | −1.04524 | 0.007499 |
| 205480_s_at | UGP2 | UDP-glucose pyrophosphorylase 2 | −1.0455 | 0.00039 |
| 202606_s_at | TLK1 | tousled-like kinase 1 | −1.04557 | 0.009381 |
| 204528_s_at | NAP1L1 | nucleosome assembly protein 1-like 1 | −1.04581 | 0.004283 |
| 208846_s_at | VDAC3 | voltage-dependent anion channel 3 | −1.04765 | 0.001739 |
| 212711_at | CAMSAP1 | calmodulin regulated spectrin-associated protein 1 | −1.0492 | 0.000505 |
| 202919_at | PREI3 | preimplantation protein 3 | −1.04921 | 6.33E−07 |
| 209022_at | STAG2 | stromal antigen 2 | −1.04975 | 0.006331 |
| 202245_at | LSS | lanosterol synthase (2,3-oxidosqualene-lanosterol cyclase) | −1.05039 | 0.004257 |
| 204045_at | TCEAL1 | transcription elongation factor A (SII)-like 1 | −1.05171 | 2.42E−05 |
| 564_at | GNA11 | guanine nucleotide binding protein (G protein), alpha 11 (Gq | −1.05224 | 0.000318 |
| | | class) | | |
| 209711_at | SLC35D1 | solute carrier family 35 (UDP-glucuronic acid/UDP-N- | −1.05306 | 0.005528 |
| | | acetylgalactosamine dual transporter), member D1 | | |
| 214281_s_at | RCHY1 | ring finger and CHY zinc finger domain containing 1 | −1.05545 | 0.004332 |
| 201478_s_at | DKC1 | dyskeratosis congenita 1, dyskerin | −1.05555 | 0.001035 |
| 213074_at | IRAK1BP1 | Interleukin-1 receptor-associated kinase 1 binding protein 1 | −1.05577 | 0.007078 |
| 200861_at | CNOT1 | CCR4-NOT transcription complex, subunit 1 | −1.05703 | 0.000994 |
| 201880_at | ARIH1 | Ariadne homolog, ubiquitin-conjugating enzyme E2 binding | −1.05774 | 0.000353 |
| | | protein, 1 (Drosophila) | | |
| 203037_s_at | MTSS1 | metastasis suppressor 1 | −1.05841 | 0.000965 |
| 218341_at | PPCS | phosphopantothenoylcysteine synthetase | −1.05851 | 0.003321 |
| 204526_s_at | TBC1D8 | TBC1 domain family, member 8 (with GRAM domain) | −1.05855 | 0.006887 |
| 218890_x_at | MRPL35 | mitochondrial ribosomal protein L35 | −1.05932 | 0.001357 |
| 212783_at | RBBP6 | retinoblastoma binding protein 6 | −1.06026 | 0.003395 |
| 201554_x_at | GYG | glycogenin | −1.06032 | 0.005156 |
| 204847_at | ZBTB11 | zinc finger and BTB domain containing 11 | −1.0604 | 0.001035 |
| 209476_at | TXNDC | thioredoxin domain containing | −1.06107 | 0.008228 |
| 213658_at | DKFZp547K1113 | Hypothetical protein DKFZp547K1113 | −1.06156 | 0.00309 |
| 201343_at | UBE2D2 | ubiquitin-conjugating enzyme E2D 2 (UBC4/5 homolog, yeast) | −1.06251 | 1.50E−05 |
| 209362_at | SURB7 | SRB7 suppressor of RNA polymerase B homolog (yeast) | −1.06264 | 0.007739 |
| 203264_s_at | ARHGEF9 | Cdc42 guanine nucleotide exchange factor (GEF) 9 | −1.06323 | 0.003278 |
| 219253_at | FAM11B | family with sequence similarity 11, member B | −1.06342 | 0.006739 |
| 213473_at | BRAP | BRCA1 associated protein | −1.06371 | 0.00688 |
| 200934_at | DEK | DEK oncogene (DNA binding) | −1.06389 | 0.005255 |
| 218680_x_at | HYPK | Huntingtin interacting protein K | −1.06405 | 0.00096 |
| 210465_s_at | SNAPC3 | small nuclear RNA activating complex, polypeptide 3, 50 kDa | −1.06558 | 0.003108 |
| 202678_at | GTF2A2 | general transcription factor IIA, 2, 12 kDa | −1.06596 | 0.000387 |
| 203224_at | RFK | riboflavin kinase | −1.06609 | 0.000285 |
| 203013_at | HSGT1 | suppressor of S. cerevisiae gcr2 | −1.06813 | 1.67E−05 |
| 203771_s_at | BLVRA | biliverdin reductase A | −1.06817 | 0.007087 |
| 219343_at | CDC37L1 | CDC37 cell division cycle 37 homolog (S. cerevisiae)-like 1 | −1.06849 | 0.002578 |
| 218645_at | ZNF277 | zinc finger protein 277 | −1.06858 | 0.007635 |
| 200853_at | H2AFZ | H2A histone family, member Z | −1.06979 | 0.000133 |
| 212026_s_at | EXOC7 | exocyst complex component 7 | −1.07141 | 0.000239 |
| 214473_x_at | PMS2L3 | postmeiotic segregation increased 2-like 3 | −1.07144 | 0.00187 |
| 212678_at | NF1 | Neurofibromin 1 (neurofibromatosis, von Recklinghausen | −1.0715 | 0.000711 |
| | | disease, Watson disease) | | |
| 218877_s_at | C6orf75 | chromosome 6 open reading frame 75 | −1.07239 | 0.004502 |
| 222014_x_at | MTO1 | mitochondrial translation optimization 1 homolog (S. cerevisiae) | −1.0737 | 3.62E−05 |
| 213227_at | PGRMC2 | progesterone receptor membrane component 2 | −1.07436 | 0.000125 |
| 208946_s_at | BECN1 | beclin 1 (coiled-coil, myosin-like BCL2 interacting protein) | −1.0755 | 0.00204 |
| 204993_at | GNAZ | guanine nucleotide binding protein (G protein), alpha z | −1.07572 | 0.002792 |
| | | polypeptide | | |
| 203486_s_at | ARMC8 | armadillo repeat containing 8 | −1.07596 | 0.00109 |
| 212103_at | KPNA6 | Karyopherin alpha 6 (importin alpha 7) | −1.07618 | 0.007635 |
| 200611_s_at | WDR1 | WD repeat domain 1 | −1.07655 | 0.000862 |
| 200740_s_at | SUMO3 | SMT3 suppressor of mif two 3 homolog 3 (yeast) | −1.077 | 0.005029 |
| 213097_s_at | ZRF1 | zuotin related factor 1 | −1.07756 | 0.001369 |
| 210470_x_at | NONO | non-POU domain containing, octamer-binding | −1.07811 | 0.004229 |
| 202382_s_at | GNPDA1 | glucosamine-6-phosphate deaminase 1 | −1.08072 | 0.006015 |
| 212163_at | KIDINS220 | kinase D-interacting substance of 220 kDa | −1.08078 | 0.003472 |
| 216111_x_at | PMS2L3 | postmeiotic segregation increased 2-like 3 | −1.08126 | 0.007033 |
| 204278_s_at | EBAG9 | estrogen receptor binding site associated, antigen, 9 | −1.08131 | 0.003141 |
| 215450_at | — | — | −1.08169 | 1.65E−05 |
| 200808_s_at | ZYX | zyxin | −1.08176 | 0.004648 |
| 205672_at | XPA | xeroderma pigmentosum, complementation group A | −1.0825 | 0.00537 |
| 208821_at | SNRPB | small nuclear ribonucleoprotein polypeptides B and B1 | −1.08266 | 0.001266 |
| 219631_at | LRP12 | low density lipoprotein-related protein 12 | −1.08296 | 0.002927 |
| 211160_x_at | ACTN1 | actinin, alpha 1 | −1.08392 | 0.004755 |
| 201181_at | GNAI3 | guanine nucleotide binding protein (G protein), alpha inhibiting | −1.08394 | 0.000329 |
| | | activity polypeptide 3 | | |
| 201685_s_at | C14orf92 | chromosome 14 open reading frame 92 | −1.0843 | 0.006662 |
| 208745_at | ATP5L | ATP synthase, H+ transporting, mitochondrial F0 complex, | −1.08603 | 0.000353 |
| | | subunit g | | |
| 208822_s_at | DAP3 | death associated protein 3 | −1.08667 | 3.19E−05 |
| 213153_at | KIAA1076 | KIAA1076 protein | −1.08823 | 0.000803 |
| 203880_at | COX17 | COX17 homolog, cytochrome c oxidase assembly protein (yeast) | −1.08836 | 0.00149 |
| 210268_at | NFX1 | nuclear transcription factor, X-box binding 1 | −1.08858 | 0.007529 |
| 216609_at | TXN | Thioredoxin | −1.08893 | 1.38E−05 |
| 205047_s_at | ASNS | asparagine synthetase | −1.08953 | 0.00863 |
| 206174_s_at | PPP6C | protein phosphatase 6, catalytic subunit | −1.09007 | 0.006505 |
| 37996_s_at | DMPK | dystrophia myotonica-protein kinase | −1.09023 | 0.006986 |
| 221613_s_at | ZA20D3 | zinc finger, A20 domain containing 3 | −1.09028 | 1.33E−05 |
| 201941_at | CPD | carboxypeptidase D | −1.09056 | 0.001236 |
| AFFX- | GAPDH | glyceraldehyde-3-phosphate dehydrogenase | −1.09154 | 0.004355 |
| HUMGAPDH/ | | | | |
| M33197_5_at | | | | |
| 202179_at | BLMH | bleomycin hydrolase | −1.09232 | 0.008499 |
| 214975_s_at | MTMR1 | myotubularin related protein 1 | −1.09252 | 0.005417 |
| 217805_at | ILF3 | interleukin enhancer binding factor 3, 90 kDa | −1.09308 | 4.53E−08 |
| 218291_at | MAPBPIP | mitogen-activated protein-binding protein-interacting protein | −1.09323 | 4.95E−05 |
| 207769_s_at | PQBP1 | polyglutamine binding protein 1 | −1.09328 | 0.005063 |
| 219158_s_at | NARG1 | NMDA receptor regulated 1 | −1.09443 | 0.001876 |
| 220631_at | OSGEPL1 | O-sialoglycoprotein endopeptidase-like 1 | −1.09568 | 0.000665 |
| 209592_s_at | WDR68 | WD repeat domain 68 | −1.09593 | 0.002221 |
| 200731_s_at | PTP4A1 | protein tyrosine phosphatase type IVA, member 1 | −1.09626 | 0.009675 |
| 217773_s_at | NDUFA4 | NADH dehydrogenase (ubiquinone) 1 alpha subcomplex, 4, | −1.09694 | 0.005703 |
| | | 9 kDa | | |
| 208761_s_at | SUMO1 | SMT3 suppressor of mif two 3 homolog 1 (yeast) | −1.10083 | 5.02E−05 |
| 208805_at | PSMA6 | proteasome (prosome, macropain) subunit, alpha type, 6 | −1.10187 | 0.000471 |
| 208633_s_at | MACF1 | microtubule-actin crosslinking factor 1 | −1.10259 | 0.001311 |
| 205231_s_at | EPM2A | epilepsy, progressive myoclonus type 2A, Lafora disease | −1.10311 | 2.48E−06 |
| | | (laforin) | | |
| 218095_s_at | TPARL | TPA regulated locus | −1.10321 | 0.000755 |
| 201636_at | FXR1 | fragile X mental retardation, autosomal homolog 1 | −1.10328 | 0.008648 |
| 220094_s_at | C6orf79 | chromosome 6 open reading frame 79 | −1.10366 | 0.001236 |
| 205547_s_at | TAGLN | transgelin | −1.10372 | 0.0031 |
| 203008_x_at | TXNDC9 | thioredoxin domain containing 9 | −1.10454 | 0.000201 |
| 201546_at | TRIP12 | thyroid hormone receptor interactor 12 | −1.10499 | 0.000104 |
| 214662_at | WDR43 | WD repeat domain 43 | −1.10534 | 0.006091 |
| 202026_at | SDHD | succinate dehydrogenase complex, subunit D, integral | −1.10565 | 0.00135 |
| | | membrane protein | | |
| 204149_s_at | GSTM4 | glutathione S-transferase M4 | −1.10602 | 0.008214 |
| 210852_s_at | AASS | aminoadipate-semialdehyde synthase | −1.10621 | 0.000818 |
| 209748_at | SPAST | spastin | −1.10685 | 0.002077 |
| 202543_s_at | GMFB | glia maturation factor, beta | −1.1072 | 0.006161 |
| 212736_at | C16orf45 | chromosome 16 open reading frame 45 | −1.10733 | 0.005944 |
| 213694_at | RSBN1 | round spermatid basic protein 1 | −1.1074 | 0.000244 |
| 202225_at | CRK | V-crk sarcoma virus CT10 oncogene homolog (avian) | −1.10922 | 0.000113 |
| 201948_at | GNL2 | guanine nucleotide binding protein-like 2 (nucleolar) | −1.10959 | 0.004237 |
| 201535_at | UBL3 | ubiquitin-like 3 | −1.11009 | 0.00134 |
| 201155_s_at | MFN2 | mitofusin 2 | −1.11021 | 2.73E−05 |
| 218226_s_at | NDUFB4 | NADH dehydrogenase (ubiquinone) 1 beta subcomplex, 4, | −1.11065 | 0.005333 |
| | | 15 kDa | | |
| 217987_at | NBLA00058 | putative protein product of Nbla00058 | −1.11218 | 0.000607 |
| 218417_s_at | FLJ20489 | hypothetical protein FLJ20489 | −1.11232 | 0.000156 |
| 213864_s_at | NAP1L1 | nucleosome assembly protein 1-like 1 | −1.11233 | 0.000874 |
| 218768_at | NUP107 | nucleoporin 107 kDa | −1.11339 | 0.000243 |
| 203492_x_at | PIG8 | translokin | −1.11369 | 0.007137 |
| 218884_s_at | FLJ13220 | hypothetical protein FLJ13220 | −1.11396 | 4.85E−08 |
| 218536_at | MRS2L | MRS2-like, magnesium homeostasis factor (S. cerevisiae) | −1.11399 | 2.44E−05 |
| 205709_s_at | CDS1 | CDP-diacylglycerol synthase (phosphatidate cytidylyltransferase) 1 | −1.11529 | 0.001962 |
| 217927_at | SPCS1 | signal peptidase complex subunit 1 homolog (S. cerevisiae) | −1.11576 | 0.000631 |
| 208864_s_at | TXN | thioredoxin | −1.11585 | 0.001026 |
| 221550_at | COX15 | COX15 homolog, cytochrome c oxidase assembly protein (yeast) | −1.11831 | 4.27E−05 |
| 201707_at | PEX19 | peroxisomal biogenesis factor 19 | −1.11851 | 3.84E−05 |
| 201681_s_at | DLG5 | discs, large homolog 5 (Drosophila) | −1.11918 | 0.009142 |
| 201275_at | FDPS | farnesyl diphosphate synthase (farnesyl pyrophosphate | −1.11985 | 0.000263 |
| | | synthetase, dimethylallyltranstransferase, | | |
| | | geranyltranstransferase) | | |
| 210293_s_at | SEC23B | Sec23 homolog B (S. cerevisiae) | −1.12079 | 0.004182 |
| 213149_at | DLAT | dihydrolipoamide S-acetyltransferase (E2 component of pyruvate | −1.12181 | 0.00014 |
| | | dehydrogenase complex) | | |
| 200755_s_at | CALU | calumenin | −1.12359 | 0.007027 |
| 218265_at | SECISBP2 | SECIS binding protein 2 | −1.12408 | 0.002584 |
| 221748_s_at | TNS1 | tensin 1 /// tensin 1 | −1.12488 | 0.006902 |
| 209080_x_at | TXNL2 | thioredoxin-like 2 | −1.12533 | 0.005541 |
| 204185_x_at | PPID | peptidylprolyl isomerase D (cyclophilin D) | −1.12575 | 0.000128 |
| 221900_at | COL8A2 | collagen, type VIII, alpha 2 | −1.12627 | 0.00606 |
| 212474_at | KIAA0241 | KIAA0241 protein | −1.12711 | 0.001705 |
| 211569_s_at | HADHSC | L-3-hydroxyacyl-Coenzyme A dehydrogenase, short chain | −1.12736 | 0.003663 |
| 212215_at | PREPL | prolyl endopeptidase-like | −1.1282 | 0.001168 |
| 202469_s_at | CPSF6 | cleavage and polyadenylation specific factor 6, 68 kDa | −1.12902 | 0.007076 |
| 218462_at | BXDC5 | brix domain containing 5 | −1.1296 | 7.48E−06 |
| 202593_s_at | MIR16 | membrane interacting protein of RGS16 | −1.13123 | 0.006115 |
| 209694_at | PTS | 6-pyruvoyltetrahydropterin synthase | −1.13273 | 0.000926 |
| 212072_s_at | CSNK2A1 | casein kinase 2, alpha 1 polypeptide | −1.13384 | 0.008511 |
| 212575_at | C19orf6 | chromosome 19 open reading frame 6 | −1.13403 | 0.000259 |
| 201068_s_at | PSMC2 | proteasome (prosome, macropain) 26S subunit, ATPase, 2 | −1.13486 | 2.50E−06 |
| 211275_s_at | GYG | glycogenin | −1.13712 | 0.00262 |
| 218487_at | ALAD | aminolevulinate, delta-, dehydratase | −1.13979 | 0.001043 |
| 203199_s_at | MTRR | 5-methyltetrahydrofolate-homocysteine methyltransferase | −1.13981 | 0.006796 |
| | | reductase | | |
| 214545_s_at | PROSC | proline synthetase co-transcribed homolog (bacterial) | −1.1401 | 0.001522 |
| 218386_x_at | USP16 | ubiquitin specific peptidase 16 | −1.14064 | 2.37E−05 |
| 212676_at | NF1 | Neurofibromin 1 (neurofibromatosis, von Recklinghausen | −1.14075 | 0.007128 |
| | | disease, Watson disease) | | |
| 203484_at | SEC61G | Sec61 gamma subunit | −1.1431 | 0.001061 |
| 214105_at | SOCS3 | suppressor of cytokine signaling 3 | −1.14372 | 0.001218 |
| 219489_s_at | NXN | nucleoredoxin | −1.1438 | 0.003751 |
| 205565_s_at | FXN | frataxin | −1.1444 | 0.002 |
| 200750_s_at | RAN | RAN, member RAS oncogene family | −1.14476 | 0.000277 |
| 204793_at | GPRASP1 | G protein-coupled receptor associated sorting protein 1 | −1.14494 | 0.002761 |
| 202511_s_at | ATG5 | ATG5 autophagy related 5 homolog (S. cerevisiae) | −1.14507 | 0.008464 |
| 215772_x_at | SUCLG2 | succinate-CoA ligase, GDP-forming, beta subunit | −1.14601 | 0.006876 |
| 202089_s_at | SLC39A6 | solute carrier family 39 (zinc transporter), member 6 | −1.14655 | 0.002144 |
| 203746_s_at | HCCS | holocytochrome c synthase (cytochrome c heme-lyase) | −1.14694 | 0.005857 |
| 201585_s_at | SFPQ | splicing factor proline/glutamine-rich (polypyrimidine tract binding | −1.14706 | 0.003225 |
| | | protein associated) | | |
| 211960_s_at | RAB7 | RAB7, member RAS oncogene family | −1.14779 | 0.002564 |
| 212293_at | HIPK1 | homeodomain interacting protein kinase 1 | −1.14862 | 6.50E−05 |
| 220282_at | RIC3 | resistance to inhibitors of cholinesterase 3 homolog (C. elegans) | −1.14925 | 0.000241 |
| 212027_at | RBM25 | RNA binding motif protein 25 | −1.14944 | 0.008825 |
| 210425_x_at | GOLGA8B | golgi autoantigen, golgin subfamily a, 8B | −1.14963 | 0.00447 |
| 200777_s_at | BZW1 | basic leucine zipper and W2 domains 1 | −1.1501 | 0.001973 |
| 200848_at | AHCYL1 | S-adenosylhomocysteine hydrolase-like 1 | −1.15156 | 4.42E−05 |
| 201899_s_at | UBE2A | ubiquitin-conjugating enzyme E2A (RAD6 homolog) | −1.15218 | 8.13E−06 |
| 215392_at | USP3 | Ubiquitin specific peptidase 3 | −1.15369 | 0.004248 |
| 208654_s_at | CD164 | CD164 antigen, sialomucin | −1.15384 | 0.009458 |
| 221502_at | KPNA3 | karyopherin alpha 3 (importin alpha 4) | −1.15575 | 0.000889 |
| 218323_at | RHOT1 | ras homolog gene family, member T1 | −1.15591 | 2.03E−05 |
| 210046_s_at | IDH2 | isocitrate dehydrogenase 2 (NADP+), mitochondrial | −1.15612 | 0.006787 |
| 212914_at | CBX7 | chromobox homolog 7 | −1.1568 | 0.004698 |
| 204342_at | SLC25A24 | solute carrier family 25 (mitochondrial carrier; phosphate carrier), | −1.15685 | 0.003497 |
| | | member 24 | | |
| 204186_s_at | PPID | peptidylprolyl isomerase D (cyclophilin D) | −1.15706 | 0.009905 |
| 215359_x_at | ZNF44 | zinc finger protein 44 (KOX 7) | −1.15847 | 0.000168 |
| 200692_s_at | HSPA9B | heat shock 70 kDa protein 9B (mortalin-2) | −1.16174 | 0.003464 |
| 201437_s_at | EIF4E | eukaryotic translation initiation factor 4E | −1.16221 | 0.007595 |
| 205132_at | ACTC | actin, alpha, cardiac muscle | −1.16234 | 0.000352 |
| 201816_s_at | GBAS | glioblastoma amplified sequence | −1.16236 | 0.000572 |
| 209788_s_at | ARTS-1 | type 1 tumor necrosis factor receptor shedding aminopeptidase | −1.16315 | 0.002779 |
| | | regulator | | |
| 221761_at | ADSS | adenylosuccinate synthase | −1.16369 | 0.000137 |
| 209095_at | DLD | dihydrolipoamide dehydrogenase (E3 component of pyruvate | −1.16379 | 0.000459 |
| | | dehydrogenase complex, 2-oxo-glutarate complex, branched | | |
| | | chain keto acid dehydrogenase complex) | | |
| 205087_at | RWDD3 | RWD domain containing 3 | −1.1641 | 0.005736 |
| 214629_x_at | RTN4 | reticulon 4 | −1.16428 | 0.000592 |
| 203494_s_at | PIG8 | translokin | −1.16436 | 0.009852 |
| 217797_at | UFC1 | ubiquitin-fold modifier conjugating enzyme 1 | −1.16451 | 5.10E−06 |
| 219571_s_at | ZNF12 | zinc finger protein 12 | −1.16484 | 5.77E−05 |
| 210296_s_at | PXMP3 | peroxisomal membrane protein 3, 35 kDa (Zellweger syndrome) | −1.16546 | 1.68E−05 |
| 200096_s_at | ATP6V0E | ATPase, H+ transporting, lysosomal 9 kDa, V0 subunit e /// | −1.1656 | 7.29E−05 |
| | | ATPase, H+ transporting, lysosomal 9 kDa, V0 subunit e | | |
| 217972_at | CHCHD3 | coiled-coil-helix-coiled-coil-helix domain containing 3 | −1.16615 | 3.47E−06 |
| 212301_at | RTF1 | Rtf1, Paf1/RNA polymerase II complex component, homolog | −1.16656 | 0.00027 |
| | | (S. cerevisiae) | | |
| 214172_x_at | RYK | RYK receptor-like tyrosine kinase | −1.16741 | 0.000141 |
| 214790_at | SENP6 | SUMO1/sentrin specific peptidase 6 | −1.16827 | 0.006842 |
| 212900_at | SEC24A | SEC24 related gene family, member A (S. cerevisiae) | −1.16899 | 0.000879 |
| 213822_s_at | UBE3B | ubiquitin protein ligase E3B | −1.1692 | 0.004571 |
| 219644_at | NY-REN-58 | NY-REN-58 antigen | −1.16932 | 0.003829 |
| 211729_x_at | BLVRA | biliverdin reductase A /// biliverdin reductase A | −1.16955 | 0.000399 |
| 209858_x_at | MPPE1 | metallophosphoesterase 1 | −1.17012 | 0.001427 |
| 204831_at | CDK8 | Cyclin-dependent kinase 8 | −1.17025 | 2.53E−08 |
| 203775_at | SLC25A13 | solute carrier family 25, member 13 (citrin) | −1.1713 | 6.68E−05 |
| 210132_at | EFNA3 | ephrin-A3 | −1.17158 | 0.001826 |
| 214027_x_at | DES /// FAM48A | desmin /// family with sequence similarity 48, member A | −1.172 | 0.001362 |
| 221510_s_at | GLS | glutaminase | −1.17218 | 0.003961 |
| 204634_at | NEK4 | NIMA (never in mitosis gene a)-related kinase 4 | −1.17248 | 0.001548 |
| 201407_s_at | PPP1CB | protein phosphatase 1, catalytic subunit, beta isoform | −1.17307 | 0.004318 |
| 202894_at | EPHB4 | EPH receptor B4 | −1.17519 | 0.004195 |
| 202634_at | POLR2K | polymerase (RNA) II (DNA directed) polypeptide K, 7.0 kDa | −1.17611 | 0.000277 |
| 213308_at | SHANK2 | SH3 and multiple ankyrin repeat domains 2 | −1.17648 | 0.00147 |
| 212050_at | WIRE | WIRE protein | −1.17739 | 0.000153 |
| 209898_x_at | ITSN2 | intersectin 2 | −1.17763 | 1.96E−05 |
| 218989_x_at | SLC30A5 | solute carrier family 30 (zinc transporter), member 5 | −1.17817 | 0.00309 |
| 204083_s_at | TPM2 | tropomyosin 2 (beta) | −1.17886 | 0.000355 |
| 208778_s_at | TCP1 | t-complex 1 | −1.17944 | 1.62E−06 |
| 204461_x_at | RAD1 | RAD1 homolog (S. pombe) | −1.1817 | 0.00022 |
| 202251_at | PRPF3 | PRP3 pre-mRNA processing factor 3 homolog (yeast) | −1.18171 | 0.004284 |
| 201921_at | GNG10 /// | guanine nucleotide binding protein (G protein), gamma 10 /// | −1.18404 | 0.002504 |
| | LOC552891 | hypothetical protein LOC552891 | | |
| 200911_s_at | TACC1 | transforming, acidic coiled-coil containing protein 1 | −1.1848 | 0.000278 |
| 208779_x_at | DDR1 | discoidin domain receptor family, member 1 | −1.1848 | 0.000751 |
| 201472_at | VBP1 | von Hippel-Lindau binding protein 1 | −1.18572 | 0.000133 |
| 217851_s_at | C20orf45 | chromosome 20 open reading frame 45 | −1.18657 | 0.001396 |
| 201317_s_at | PSMA2 | proteasome (prosome, macropain) subunit, alpha type, 2 | −1.18674 | 0.003149 |
| 219871_at | FLJ13197 | hypothetical protein FLJ13197 | −1.18756 | 0.002808 |
| 212920_at | — | — | −1.18766 | 0.00201 |
| 202558_s_at | STCH | stress 70 protein chaperone, microsome-associated, 60 kDa | −1.19024 | 0.000173 |
| 215387_x_at | GPC6 | Glypican 6 | −1.19077 | 0.002279 |
| 212740_at | PIK3R4 | phosphoinositide-3-kinase, regulatory subunit 4, p150 | −1.19204 | 0.003918 |
| 213083_at | SLC35D2 | solute carrier family 35, member D2 | −1.19365 | 0.000111 |
| 217496_s_at | IDE | insulin-degrading enzyme | −1.19398 | 0.005979 |
| 202821_s_at | LPP | LIM domain containing preferred translocation partner in lipoma | −1.19617 | 0.00372 |
| 214077_x_at | MEIS4 | Meis1, myeloid ecotropic viral integration site 1 homolog 4 | −1.19628 | 0.00023 |
| | | (mouse) | | |
| 215936_s_at | KIAA1033 | KIAA1033 | −1.19727 | 0.002638 |
| 211501_s_at | EIF3S9 | eukaryotic translation initiation factor 3, subunit 9 eta, 116 kDa | −1.19764 | 7.15E−07 |
| 213508_at | C14orf147 | chromosome 14 open reading frame 147 | −1.19875 | 0.000586 |
| 205110_s_at | FGF13 | fibroblast growth factor 13 | −1.19974 | 0.002879 |
| 219327_s_at | GPRC5C | G protein-coupled receptor, family C, group 5, member C | −1.19974 | 0.000176 |
| 203954_x_at | CLDN3 | claudin 3 | −1.19985 | 0.000715 |
| 209084_s_at | RAB28 | RAB28, member RAS oncogene family | −1.20017 | 1.22E−05 |
| 200646_s_at | NUCB1 | nucleobindin 1 | −1.20195 | 0.005081 |
| 55081_at | MICAL-L1 | MICAL-like 1 | −1.20522 | 0.000107 |
| 208310_s_at | C7orf28A /// | chromosome 7 open reading frame 28A /// chromosome 7 open | −1.20808 | 0.000916 |
| | C7orf28B | reading frame 28B | | |
| 214113_s_at | RBM8A | RNA binding motif protein 8A | −1.20844 | 0.004669 |
| 201058_s_at | MYL9 | myosin, light polypeptide 9, regulatory | −1.20932 | 0.000168 |
| 202066_at | PPFIA1 | protein tyrosine phosphatase, receptor type, f polypeptide | −1.20983 | 0.004522 |
| | | (PTPRF), interacting protein (liprin), alpha 1 | | |
| 209003_at | SLC25A11 | solute carrier family 25 (mitochondrial carrier; oxoglutarate | −1.21014 | 0.001107 |
| | | carrier), member 11 | | |
| 202617_s_at | MECP2 | methyl CpG binding protein 2 (Rett syndrome) | −1.21173 | 0.000248 |
| 201326_at | CCT6A | chaperonin containing TCP1, subunit 6A (zeta 1) | −1.21234 | 0.007532 |
| 219275_at | PDCD5 | programmed cell death 5 | −1.21601 | 0.001281 |
| 202353_s_at | PSMD12 | proteasome (prosome, macropain) 26S subunit, non-ATPase, 12 | −1.21652 | 4.12E−08 |
| 200897_s_at | KIAA0992 | palladin | −1.21727 | 0.000326 |
| 219362_at | MAK10 | MAK10 homolog, amino-acid N-acetyltransferase subunit, | −1.22088 | 0.002407 |
| | | (S. cerevisiae) | | |
| 206555_s_at | THUMPD1 | THUMP domain containing 1 | −1.22129 | 0.001063 |
| 208991_at | STAT3 | signal transducer and activator of transcription 3 (acute-phase | −1.22142 | 1.43E−06 |
| | | response factor) | | |
| 210830_s_at | PON2 | paraoxonase 2 | −1.22354 | 0.002012 |
| 204248_at | GNA11 | guanine nucleotide binding protein (G protein), alpha 11 (Gq | −1.22529 | 0.000199 |
| | | class) | | |
| 202346_at | HIP2 | huntingtin interacting protein 2 | −1.22612 | 0.00722 |
| 216060_s_at | DAAM1 | dishevelled associated activator of morphogenesis 1 | −1.22679 | 0.001094 |
| 218761_at | RNF111 | ring finger protein 111 | −1.22723 | 0.000358 |
| 217958_at | TRAPPC4 | trafficking protein particle complex 4 | −1.22834 | 1.92E−05 |
| 200621_at | CSRP1 | cysteine and glycine-rich protein 1 | −1.22861 | 5.18E−07 |
| 201699_at | PSMC6 | proteasome (prosome, macropain) 26S subunit, ATPase, 6 | −1.22894 | 0.003036 |
| 200989_at | HIF1A | hypoxia-inducible factor 1, alpha subunit (basic helix-loop-helix | −1.22957 | 0.000378 |
| | | transcription factor) | | |
| 218204_s_at | FYCO1 | FYVE and coiled-coil domain containing 1 | −1.2331 | 0.008416 |
| 218139_s_at | C14orf108 | chromosome 14 open reading frame 108 | −1.23343 | 0.001325 |
| 202798_at | SEC24B | SEC24 related gene family, member B (S. cerevisiae) | −1.23414 | 0.004852 |
| 212453_at | KIAA1279 | KIAA1279 | −1.23492 | 0.003273 |
| 201371_s_at | CUL3 | cullin 3 | −1.2351 | 7.02E−06 |
| 209799_at | PRKAA1 | protein kinase, AMP-activated, alpha 1 catalytic subunit | −1.23645 | 0.002616 |
| 202797_at | SACM1L | SAC1 suppressor of actin mutations 1-like (yeast) | −1.23778 | 0.003388 |
| 200626_s_at | MATR3 | matrin 3 | −1.2381 | 0.004564 |
| 212428_at | KIAA0368 | KIAA0368 | −1.24019 | 2.92E−06 |
| 211276_at | TCEAL2 | transcription elongation factor A (SII)-like 2 | −1.24057 | 0.006791 |
| 201424_s_at | CUL4A | cullin 4A | −1.24187 | 0.0023 |
| 204640_s_at | SPOP | speckle-type POZ protein | −1.242 | 0.000759 |
| 200883_at | UQCRC2 | ubiquinol-cytochrome c reductase core protein II | −1.24246 | 0.000986 |
| 203370_s_at | PDLIM7 | PDZ and LIM domain 7 (enigma) | −1.24284 | 0.001384 |
| 219297_at | WDR44 | WD repeat domain 44 | −1.24292 | 0.000531 |
| 209454_s_at | TEAD3 | TEA domain family member 3 | −1.24307 | 7.04E−05 |
| 211505_s_at | STAU | staufen, RNA binding protein (Drosophila) | −1.24324 | 0.000594 |
| 202056_at | KPNA1 | Karyopherin alpha 1 (importin alpha 5) | −1.2441 | 1.23E−05 |
| 202352_s_at | PSMD12 | proteasome (prosome, macropain) 26S subunit, non-ATPase, 12 | −1.24413 | 1.31E−05 |
| 216205_s_at | MFN2 | mitofusin 2 | −1.24429 | 0.002155 |
| 202749_at | WRB | tryptophan rich basic protein | −1.24545 | 0.009232 |
| 202147_s_at | IFRD1 | interferon-related developmental regulator 1 | −1.24588 | 0.005187 |
| 201889_at | FAM3C | family with sequence similarity 3, member C | −1.24625 | 0.003784 |
| 213708_s_at | MLX | MAX-like protein X | −1.24751 | 0.000232 |
| 202432_at | PPP3CB | protein phosphatase 3 (formerly 2B), catalytic subunit, beta | −1.24831 | 4.59E−05 |
| | | isoform (calcineurin A beta) | | |
| 207573_x_at | ATP5L | ATP synthase, H+ transporting, mitochondrial F0 complex, | −1.24847 | 3.04E−08 |
| | | subunit g | | |
| 220746_s_at | RAP80 | receptor associated protein 80 | −1.24917 | 1.21E−06 |
| 206976_s_at | HSPH1 | heat shock 105 kDa/110 kDa protein 1 | −1.24949 | 0.006241 |
| 212722_s_at | PTDSR | phosphatidylserine receptor | −1.24973 | 0.000169 |
| 202811_at | STAMBP | STAM binding protein | −1.24974 | 7.55E−05 |
| 212751_at | UBE2N | ubiquitin-conjugating enzyme E2N (UBC13 homolog, yeast) | −1.24976 | 0.000206 |
| 202177_at | GAS6 | growth arrest-specific 6 | −1.25042 | 0.000498 |
| 217816_s_at | PCNP | PEST-containing nuclear protein | −1.25075 | 2.12E−05 |
| 203531_at | CUL5 | cullin 5 | −1.25532 | 1.39E−05 |
| 204119_s_at | ADK | adenosine kinase | −1.25669 | 5.75E−05 |
| 200854_at | NCOR1 | nuclear receptor co-repressor 1 | −1.25716 | 0.000749 |
| 201586_s_at | SFPQ | splicing factor proline/glutamine-rich (polypyrimidine tract binding | −1.25966 | 0.000697 |
| | | protein associated) | | |
| 216511_s_at | TCF7L2 | transcription factor 7-like 2 (T-cell specific, HMG-box) | −1.26109 | 0.006526 |
| 203682_s_at | IVD | isovaleryl Coenzyme A dehydrogenase | −1.26134 | 0.000945 |
| 200859_x_at | FLNA | filamin A, alpha (actin binding protein 280) | −1.26153 | 0.000758 |
| 211382_s_at | TACC2 | transforming, acidic coiled-coil containing protein 2 | −1.26228 | 0.004461 |
| 213005_s_at | ANKRD15 | ankyrin repeat domain 15 | −1.26326 | 0.002195 |
| 212217_at | PREPL | prolyl endopeptidase-like | −1.26496 | 0.003528 |
| 203970_s_at | PEX3 | peroxisomal biogenesis factor 3 | −1.26526 | 0.000719 |
| 204517_at | PPIC | peptidylprolyl isomerase C (cyclophilin C) | −1.26571 | 0.001784 |
| 209063_x_at | PAIP1 | poly(A) binding protein interacting protein 1 | −1.26728 | 0.001655 |
| 204091_at | PDE6D | phosphodiesterase 6D, cGMP-specific, rod, delta | −1.2674 | 7.79E−08 |
| 204658_at | TRA2A | transformer-2 alpha | −1.26785 | 0.001081 |
| 208698_s_at | NONO | non-POU domain containing, octamer-binding | −1.26796 | 0.003379 |
| 201733_at | CLCN3 | chloride channel 3 | −1.26836 | 0.002096 |
| 220607_x_at | TH1L | TH1-like (Drosophila) | −1.26837 | 0.002901 |
| 210878_s_at | JMJD1B | jumonji domain containing 1B | −1.27004 | 0.000692 |
| 201975_at | RSN | restin (Reed-Steinberg cell-expressed intermediate filament- | −1.27196 | 6.32E−06 |
| | | associated protein) | | |
| 209865_at | SLC35A3 | solute carrier family 35 (UDP-N-acetylglucosamine (UDP- | −1.27257 | 0.000993 |
| | | GlcNAc) transporter), member A3 | | |
| 213883_s_at | TM2D1 | TM2 domain containing 1 | −1.2726 | 0.006966 |
| 202337_at | PMF1 | polyamine-modulated factor 1 | −1.27265 | 6.10E−12 |
| 205763_s_at | DDX18 | DEAD (Asp-Glu-Ala-Asp) box polypeptide 18 | −1.27353 | 0.001804 |
| 212442_s_at | LASS6 | LAG1 longevity assurance homolog 6 (S. cerevisiae) | −1.27456 | 6.73E−05 |
| 200662_s_at | TOMM20 | translocase of outer mitochondrial membrane 20 homolog | −1.27557 | 0.000673 |
| | | (yeast) | | |
| 210096_at | CYP4B1 | cytochrome P450, family 4, subfamily B, polypeptide 1 | −1.27774 | 1.67E−05 |
| 221891_x_at | HSPA8 | heat shock 70 kDa protein 8 | −1.27794 | 0.000122 |
| 201277_s_at | HNRPAB | heterogeneous nuclear ribonucleoprotein A/B | −1.27833 | 5.83E−07 |
| 212813_at | JAM3 | junctional adhesion molecule 3 | −1.27868 | 0.003861 |
| 211317_s_at | CFLAR | CASP8 and FADD-like apoptosis regulator | −1.27876 | 0.002759 |
| 219880_at | — | — | −1.27936 | 0.000852 |
| 210045_at | IDH2 | isocitrate dehydrogenase 2 (NADP+), mitochondrial | −1.27985 | 0.005488 |
| 201316_at | PSMA2 | proteasome (prosome, macropain) subunit, alpha type, 2 | −1.28041 | 0.000631 |
| 208809_s_at | C6orf62 | chromosome 6 open reading frame 62 | −1.28087 | 0.001527 |
| 219806_s_at | FN5 | FN5 protein | −1.28192 | 0.004427 |
| 209520_s_at | NCBP1 | nuclear cap binding protein subunit 1, 80 kDa | −1.28397 | 0.000239 |
| 218209_s_at | P15RS | hypothetical protein FLJ10656 | −1.28453 | 0.00083 |
| 203732_at | TRIP4 | thyroid hormone receptor interactor 4 | −1.28625 | 0.000711 |
| 214718_at | GATAD1 | GATA zinc finger domain containing 1 | −1.28671 | 0.000582 |
| 218533_s_at | UCKL1 | uridine-cytidine kinase 1-like 1 | −1.28717 | 4.36E−07 |
| 204992_s_at | PFN2 | profilin 2 | −1.2874 | 0.000141 |
| 212361_s_at | ATP2A2 | ATPase, Ca++ transporting, cardiac muscle, slow twitch 2 | −1.28759 | 0.000363 |
| 218474_s_at | KCTD5 | potassium channel tetramerisation domain containing 5 | −1.28923 | 4.18E−08 |
| 207480_s_at | MEIS2 | Meis1, myeloid ecotropic viral integration site 1 homolog 2 | −1.2893 | 0.001395 |
| | | (mouse) | | |
| 218519_at | SLC35A5 | solute carrier family 35, member A5 | −1.29082 | 2.01E−05 |
| 215032_at | RREB1 | ras responsive element binding protein 1 | −1.29173 | 0.000339 |
| 212302_at | RTF1 | Rtf1, Paf1/RNA polymerase II complex component, homolog | −1.29241 | 0.000509 |
| | | (S. cerevisiae) | | |
| 201025_at | EIF5B | eukaryotic translation initiation factor 5B | −1.2935 | 2.63E−07 |
| 201223_s_at | RAD23B | RAD23 homolog B (S. cerevisiae) | −1.29577 | 1.41E−05 |
| 218545_at | FLJ11088 | GGA binding partner | −1.29586 | 0.005292 |
| 218396_at | VPS13C | vacuolar protein sorting 13C (yeast) | −1.29867 | 0.000258 |
| 217909_s_at | MLX | MAX-like protein X | −1.2991 | 0.001874 |
| 202900_s_at | NUP88 | nucleoporin 88 kDa | −1.29988 | 0.001086 |
| 200700_s_at | KDELR2 | KDEL (Lys-Asp-Glu-Leu) endoplasmic reticulum protein retention | −1.29992 | 0.00042 |
| | | receptor 2 | | |
| 201549_x_at | JARID1B | Jumonji, AT rich interactive domain 1B (RBP2-like) | −1.30082 | 0.002549 |
| 200712_s_at | MAPRE1 | microtubule-associated protein, RP/EB family, member 1 | −1.30102 | 0.005699 |
| 207081_s_at | PIK4CA | phosphatidylinositol 4-kinase, catalytic, alpha polypeptide | −1.3022 | 0.000622 |
| 202277_at | SPTLC1 | serine palmitoyltransferase, long chain base subunit 1 | −1.30533 | 1.08E−05 |
| 201263_at | TARS | threonyl-tRNA synthetase | −1.30577 | 0.000764 |
| 202413_s_at | USP1 | ubiquitin specific peptidase 1 | −1.3063 | 5.69E−05 |
| 221654_s_at | USP3 | ubiquitin specific peptidase 3 | −1.30706 | 0.000448 |
| 208614_s_at | FLNB | filamin B, beta (actin binding protein 278) | −1.30782 | 0.001765 |
| 212847_at | FUBP1 | Far upstream element (FUSE) binding protein 1 | −1.309 | 0.0035 |
| 211450_s_at | MSH6 | mutS homolog 6 (E. coli) | −1.30981 | 0.002597 |
| 210987_x_at | TPM1 | tropomyosin 1 (alpha) | −1.31162 | 3.57E−05 |
| 208671_at | SERINC1 | serine incorporator 1 | −1.31191 | 0.002431 |
| 212871_at | MAPKAPK5 | mitogen-activated protein kinase-activated protein kinase 5 | −1.3138 | 1.76E−06 |
| 200907_s_at | KIAA0992 | palladin | −1.31476 | 0.000487 |
| 209064_x_at | PAIP1 | poly(A) binding protein interacting protein 1 | −1.31491 | 0.000896 |
| 212288_at | FNBP1 | formin binding protein 1 | −1.31574 | 0.002738 |
| 204460_s_at | RAD1 | RAD1 homolog (S. pombe) | −1.31638 | 1.34E−07 |
| 212476_at | CENTB2 | centaurin, beta 2 | −1.31648 | 0.003618 |
| 201518_at | CBX1 | chromobox homolog 1 (HP1 beta homolog Drosophila) | −1.31679 | 0.001544 |
| 57739_at | DND1 | dead end homolog 1 (zebrafish) | −1.3174 | 0.000101 |
| 206770_s_at | SLC35A3 | solute carrier family 35 (UDP-N-acetylglucosamine (UDP- | −1.31757 | 0.001666 |
| | | GlcNAc) transporter), member A3 | | |
| 200008_s_at | GDI2 | GDP dissociation inhibitor 2 /// GDP dissociation inhibitor 2 | −1.31898 | 0.002521 |
| 212766_s_at | ISG20L2 | interferon stimulated exonuclease gene 20 kDa-like 2 | −1.31994 | 0.005626 |
| 212944_at | MRPS6 | Mitochondrial ribosomal protein S6 | −1.32022 | 0.000547 |
| 209844_at | HOXB13 | homeo box B13 | −1.3204 | 0.001005 |
| 218230_at | ARFIP1 | ADP-ribosylation factor interacting protein 1 (arfaptin 1) | −1.32118 | 0.000333 |
| 209300_s_at | NECAP1 | NECAP endocytosis associated 1 | −1.32134 | 0.00114 |
| 212242_at | TUBA1 | tubulin, alpha 1 (testis specific) | −1.32137 | 0.00104 |
| 214687_x_at | ALDOA | aldolase A, fructose-bisphosphate | −1.32329 | 0.003704 |
| 201714_at | TUBG1 | tubulin, gamma 1 | −1.32522 | 0.001788 |
| 40562_at | GNA11 | guanine nucleotide binding protein (G protein), alpha 11 (Gq | −1.32576 | 0.000637 |
| | | class) | | |
| 205768_s_at | SLC27A2 | solute carrier family 27 (fatty acid transporter), member 2 | −1.32746 | 0.005316 |
| 200097_s_at | HNRPK | heterogeneous nuclear ribonucleoprotein K /// heterogeneous | −1.32817 | 2.11E−08 |
| | | nuclear ribonucleoprotein K | | |
| 202754_at | R3HDM1 | R3H domain containing 1 | −1.32952 | 0.000304 |
| 214429_at | MTMR6 | myotubularin related protein 6 | −1.32995 | 1.77E−05 |
| 212264_s_at | KIAA0261 | KIAA0261 | −1.33316 | 0.007317 |
| 202710_at | BET1 | BET1 homolog (S. cerevisiae) | −1.33427 | 0.001023 |
| 202922_at | GCLC | glutamate-cysteine ligase, catalytic subunit | −1.3358 | 0.001015 |
| 211662_s_at | VDAC2 | voltage-dependent anion channel 2 /// voltage-dependent anion | −1.34058 | 9.44E−13 |
| | | channel 2 | | |
| 221596_s_at | DKFZP564O0523 | hypothetical protein DKFZp564O0523 | −1.34323 | 2.15E−05 |
| 207507_s_at | ATP5G3 | ATP synthase, H+ transporting, mitochondrial F0 complex, | −1.34347 | 2.49E−06 |
| | | subunit c (subunit 9) isoform 3 | | |
| 219372_at | CDV1 | carnitine deficiency-associated, expressed in ventricle 1 | −1.34543 | 4.08E−05 |
| 218395_at | ACTR6 | ARP6 actin-related protein 6 homolog (yeast) | −1.34572 | 8.05E−06 |
| 200821_at | LAMP2 | lysosomal-associated membrane protein 2 | −1.34628 | 0.000166 |
| 210962_s_at | AKAP9 | A kinase (PRKA) anchor protein (yotiao) 9 | −1.34982 | 0.002994 |
| 201486_at | RCN2 | reticulocalbin 2, EF-hand calcium binding domain | −1.35001 | 0.000739 |
| 221727_at | SUB1 /// SUB1P1 | SUB1 homolog (S. cerevisiae) /// SUB1 homolog (S. cerevisiae) | −1.35197 | 0.002261 |
| | | pseudogene 1 | | |
| 221482_s_at | ARPP-19 | cyclic AMP phosphoprotein, 19 kD | −1.35235 | 0.002257 |
| 204009_s_at | KRAS | v-Ki-ras2 Kirsten rat sarcoma viral oncogene homolog | −1.35284 | 0.000469 |
| 204177_s_at | KLHL20 | kelch-like 20 (Drosophila) | −1.35462 | 0.000261 |
| 212287_at | SUZ12 | suppressor of zeste 12 homolog (Drosophila) | −1.35469 | 9.19E−05 |
| 212355_at | KIAA0323 | KIAA0323 | −1.3558 | 0.002643 |
| 216304_x_at | YME1L1 | YME1-like 1 (S. cerevisiae) | −1.35685 | 1.98E−05 |
| 212426_s_at | YWHAQ | tyrosine 3-monooxygenase/tryptophan 5-monooxygenase | −1.35813 | 0.000482 |
| | | activation protein, theta polypeptide | | |
| 202566_s_at | SVIL | supervillin | −1.35912 | 0.001085 |
| 202131_s_at | RIOK3 | RIO kinase 3 (yeast) /// RIO kinase 3 (yeast) | −1.36032 | 3.09E−07 |
| 212723_at | PTDSR | phosphatidylserine receptor | −1.36072 | 0.000712 |
| 213440_at | RAB1A | RAB1A, member RAS oncogene family | −1.36073 | 0.001232 |
| 212461_at | AZIN1 | antizyme inhibitor 1 | −1.36174 | 0.002169 |
| 203053_at | BCAS2 | breast carcinoma amplified sequence 2 | −1.36184 | 1.00E−05 |
| 201908_at | DVL3 | dishevelled, dsh homolog 3 (Drosophila) | −1.36446 | 7.44E−06 |
| 217890_s_at | PARVA | parvin, alpha | −1.36487 | 2.59E−08 |
| 212062_at | ATP9A | ATPase, Class II, type 9A | −1.37136 | 0.003649 |
| 217832_at | SYNCRIP | synaptotagmin binding, cytoplasmic RNA interacting protein | −1.37169 | 0.00338 |
| 202289_s_at | TACC2 | transforming, acidic coiled-coil containing protein 2 | −1.37251 | 0.002718 |
| 208841_s_at | G3BP2 | Ras-GTPase activating protein SH3 domain-binding protein 2 | −1.37371 | 0.001407 |
| 222263_at | SLC35E1 | solute carrier family 35, member E1 | −1.37427 | 0.004957 |
| 207836_s_at | RBPMS | RNA binding protein with multiple splicing | −1.37598 | 0.00527 |
| 204029_at | CELSR2 | cadherin, EGF LAG seven-pass G-type receptor 2 (flamingo | −1.37682 | 1.93E−05 |
| | | homolog, Drosophila) | | |
| 201873_s_at | ABCE1 | ATP-binding cassette, sub-family E (OABP), member 1 | −1.37865 | 1.35E−05 |
| 213372_at | PAQR3 | progestin and adipoQ receptor family member III | −1.38564 | 0.000455 |
| 206272_at | SPHAR | S-phase response (cyclin-related) | −1.38691 | 0.000879 |
| 201376_s_at | HNRPF | heterogeneous nuclear ribonucleoprotein F | −1.38798 | 2.65E−05 |
| 219356_s_at | CHMP5 | chromatin modifying protein 5 | −1.38961 | 0.000227 |
| 219647_at | POPDC2 | popeye domain containing 2 | −1.39016 | 4.96E−05 |
| 216109_at | THRAP2 | Thyroid hormone receptor associated protein 2 | −1.3902 | 8.75E−10 |
| 203218_at | MAPK9 | mitogen-activated protein kinase 9 | −1.39148 | 0.001467 |
| 201501_s_at | GRSF1 | G-rich RNA sequence binding factor 1 | −1.39329 | 6.95E−05 |
| 200931_s_at | VCL | vinculin | −1.39368 | 0.000115 |
| 219137_s_at | C2orf33 | chromosome 2 open reading frame 33 | −1.39401 | 0.005441 |
| 212445_s_at | NEDD4L | neural precursor cell expressed, developmentally down- | −1.39402 | 0.000689 |
| | | regulated 4-like | | |
| 214752_x_at | FLNA | filamin A, alpha (actin binding protein 280) | −1.39415 | 2.52E−05 |
| 204516_at | ATXN7 | ataxin 7 | −1.39424 | 0.004291 |
| 219166_at | C14orf104 | chromosome 14 open reading frame 104 | −1.39449 | 0.000188 |
| 200838_at | CTSB | cathepsin B | −1.39647 | 0.001602 |
| 213727_x_at | MPPE1 | metallophosphoesterase 1 | −1.39648 | 6.28E−05 |
| 220765_s_at | LIMS2 | LIM and senescent cell antigen-like domains 2 | −1.39775 | 1.85E−05 |
| 202635_s_at | POLR2K | polymerase (RNA) II (DNA directed) polypeptide K, 7.0 kDa | −1.4013 | 0.000478 |
| 208127_s_at | SOCS5 | suppressor of cytokine signaling 5 | −1.40268 | 1.85E−09 |
| 204755_x_at | HLF | hepatic leukemia factor | −1.40458 | 0.000656 |
| 210986_s_at | TPM1 | tropomyosin 1 (alpha) | −1.40516 | 1.24E−06 |
| 218172_s_at | DERL1 | Der1-like domain family, member 1 | −1.40591 | 2.23E−05 |
| 209379_s_at | KIAA1128 | KIAA1128 | −1.40632 | 0.002476 |
| 209115_at | UBE1C | ubiquitin-activating enzyme E1C (UBA3 homolog, yeast) | −1.40712 | 0.005428 |
| 202721_s_at | GFPT1 | glutamine-fructose-6-phosphate transaminase 1 | −1.40788 | 0.008493 |
| 208498_s_at | AMY1A /// | amylase, alpha 1A; salivary /// amylase, alpha 1B; salivary /// | −1.4082 | 0.000805 |
| | AMY1B /// | amylase, alpha 1C; salivary /// amylase, alpha 2A; pancreatic /// | | |
| | AMY1C /// | amylase, alpha 2B; pancreatic | | |
| | AMY2A /// | | | |
| | AMY2B | | | |
| 221190_s_at | C18orf8 | chromosome 18 open reading frame 8 | −1.40942 | 0.000143 |
| 206989_s_at | SFRS2IP | splicing factor, arginine/serine-rich 2, interacting protein | −1.41143 | 0.000483 |
| 202541_at | SCYE1 | small inducible cytokine subfamily E, member 1 (endothelial | −1.4124 | 0.001453 |
| | | monocyte-activating) | | |
| 202228_s_at | SDFR1 | stromal cell derived factor receptor 1 | −1.41313 | 0.00125 |
| 216035_x_at | TCF7L2 | transcription factor 7-like 2 (T-cell specific, HMG-box) | −1.41875 | 0.004496 |
| 205248_at | C21orf5 | chromosome 21 open reading frame 5 | −1.41887 | 0.001162 |
| 218373_at | FTS | fused toes homolog (mouse) | −1.4195 | 1.49E−06 |
| 204256_at | ELOVL6 | ELOVL family member 6, elongation of long chain fatty acids | −1.42058 | 0.000341 |
| | | (FEN1/Elo2, SUR4/Elo3-like, yeast) | | |
| 203112_s_at | WHSC2 | Wolf-Hirschhorn syndrome candidate 2 | −1.42071 | 0.002157 |
| 202020_s_at | LANCL1 | LanC lantibiotic synthetase component C-like 1 (bacterial) | −1.42181 | 0.000848 |
| 202401_s_at | SRF | serum response factor (c-fos serum response element-binding | −1.42182 | 0.001657 |
| | | transcription factor) | | |
| 203766_s_at | LMOD1 | leiomodin 1 (smooth muscle) | −1.42239 | 1.40E−05 |
| 200927_s_at | RAB14 | RAB14, member RAS oncogene family | −1.42282 | 0.000216 |
| 202771_at | FAM38A | family with sequence similarity 38, member A | −1.4237 | 0.003362 |
| 219043_s_at | PDCL3 /// | phosducin-like 3 /// hypothetical protein FLJ12205 | −1.4245 | 1.32E−05 |
| | LOC285359 | | | |
| 203963_at | CA12 | carbonic anhydrase XII | −1.4252 | 0.000428 |
| 208096_s_at | COL21A1 | collagen, type XXI, alpha 1 /// collagen, type XXI, alpha 1 | −1.42587 | 8.65E−05 |
| 203706_s_at | FZD7 | frizzled homolog 7 (Drosophila) | −1.42588 | 2.20E−06 |
| 221423_s_at | YIPF5 | Yip1 domain family, member 5 /// Yip1 domain family, member 5 | −1.42625 | 9.47E−06 |
| 203622_s_at | LOC56902 | putatative 28 kDa protein | −1.42673 | 0.002247 |
| 200771_at | LAMC1 | laminin, gamma 1 (formerly LAMB2) | −1.42703 | 0.002984 |
| 218381_s_at | U2AF2 | U2 (RNU2) small nuclear RNA auxiliary factor 2 | −1.42841 | 0.00036 |
| 208715_at | TMCO1 | transmembrane and coiled-coil domains 1 | −1.42951 | 0.000143 |
| 213701_at | DKFZp434N2030 | hypothetical protein DKFZp434N2030 | −1.42953 | 0.005284 |
| 210768_x_at | TMCO1 | transmembrane and coiled-coil domains 1 | −1.42997 | 3.56E−13 |
| 218196_at | OSTM1 | osteopetrosis associated transmembrane protein 1 | −1.43031 | 5.64E−05 |
| 218793_s_at | SCML1 | sex comb on midleg-like 1 (Drosophila) | −1.43083 | 0.003978 |
| 218090_s_at | BRWD2 | bromodomain and WD repeat domain containing 2 | −1.43194 | 0.000226 |
| 214753_at | PFAAP5 | Hypothetical gene CG012 | −1.4329 | 0.005675 |
| 217808_s_at | MAPKAP1 | mitogen-activated protein kinase associated protein 1 | −1.43344 | 0.007257 |
| 201192_s_at | PITPNA | phosphatidylinositol transfer protein, alpha | −1.43789 | 0.004812 |
| 218683_at | PTBP2 | polypyrimidine tract binding protein 2 | −1.4379 | 0.000846 |
| 203780_at | EVA1 | epithelial V-like antigen 1 | −1.44144 | 0.006898 |
| 202440_s_at | ST5 | suppression of tumorigenicity 5 | −1.44169 | 0.000345 |
| 207824_s_at | MAZ | MYC-associated zinc finger protein (purine-binding transcription | −1.44181 | 0.004296 |
| | | factor) | | |
| 201171_at | ATP6V0E | ATPase, H+ transporting, lysosomal 9 kDa, V0 subunit e | −1.44218 | 1.24E−09 |
| 213944_x_at | GNA11 | guanine nucleotide binding protein (G protein), alpha 11 (Gq | −1.44221 | 4.90E−06 |
| | | class) | | |
| 200749_at | RAN /// | RAN, member RAS oncogene family /// similar to Ras-related | −1.44416 | 0.000114 |
| | LOC391717 | nuclear protein | | |
| 212690_at | DDHD2 | DDHD domain containing 2 | −1.44444 | 0.002761 |
| 215043_s_at | SMA3 /// SMA5 | SMA3 /// SMA5 | −1.44464 | 0.001067 |
| 200732_s_at | PTP4A1 | protein tyrosine phosphatase type IVA, member 1 | −1.44589 | 0.000183 |
| 203723_at | ITPKB | inositol 1,4,5-trisphosphate 3-kinase B | −1.44629 | 1.54E−05 |
| 217842_at | LUC7L2 | LUC7-like 2 (S. cerevisiae) | −1.44992 | 0.006273 |
| 202853_s_at | RYK | RYK receptor-like tyrosine kinase | −1.45084 | 0.000312 |
| 219067_s_at | C10orf86 | chromosome 10 open reading frame 86 | −1.45146 | 0.002493 |
| 214323_s_at | UPF3A | UPF3 regulator of nonsense transcripts homolog A (yeast) | −1.45238 | 0.000327 |
| 209444_at | RAP1GDS1 | RAP1, GTP-GDP dissociation stimulator 1 | −1.45292 | 0.000132 |
| 219675_s_at | UXS1 | UDP-glucuronate decarboxylase 1 | −1.45344 | 7.50E−07 |
| 208636_at | ACTN1 | Actinin, alpha 1 | −1.45598 | 0.000664 |
| 203336_s_at | ITGB1BP1 | integrin beta 1 binding protein 1 | −1.46087 | 3.33E−05 |
| 202165_at | PPP1R2 | protein phosphatase 1, regulatory (inhibitor) subunit 2 | −1.46147 | 1.75E−06 |
| 209287_s_at | CDC42EP3 | CDC42 effector protein (Rho GTPase binding) 3 | −1.46211 | 0.000143 |
| 219431_at | ARHGAP10 | Rho GTPase activating protein 10 | −1.46217 | 0.000308 |
| 213024_at | TMF1 | TATA element modulatory factor 1 | −1.46264 | 7.56E−07 |
| 212205_at | H2AFV | H2A histone family, member V | −1.46293 | 5.22E−05 |
| 203414_at | MMD | monocyte to macrophage differentiation-associated | −1.46371 | 3.06E−06 |
| 214505_s_at | FHL1 | four and a half LIM domains 1 | −1.46963 | 0.001564 |
| 208754_s_at | NAP1L1 | nucleosome assembly protein 1-like 1 | −1.47112 | 7.58E−05 |
| 211754_s_at | SLC25A17 | solute carrier family 25 (mitochondrial carrier; peroxisomal | −1.47293 | 7.89E−09 |
| | | membrane protein, 34 kDa), member 17 /// solute carrier family | | |
| | | 25 (mitochondrial carrier; peroxisomal membrane protein, | | |
| | | 34 kDa), member 17 | | |
| 204068_at | STK3 | serine/threonine kinase 3 (STE20 homolog, yeast) | −1.47439 | 0.00426 |
| 202173_s_at | ZNF161 | zinc finger protein 161 | −1.47582 | 0.002718 |
| 219399_at | LIN7C | lin-7 homolog C (C. elegans) | −1.47938 | 0.003132 |
| 200996_at | ACTR3 | ARP3 actin-related protein 3 homolog (yeast) | −1.47944 | 0.000149 |
| 209817_at | PPP3CB | protein phosphatase 3 (formerly 2B), catalytic subunit, beta | −1.47959 | 0.000187 |
| | | isoform (calcineurin A beta) | | |
| 212958_x_at | PAM | peptidylglycine alpha-amidating monooxygenase | −1.48644 | 0.003593 |
| 218465_at | TMEM33 | transmembrane protein 33 | −1.48656 | 1.33E−05 |
| 203202_at | HRB2 | HIV-1 rev binding protein 2 | −1.48709 | 0.000924 |
| 221918_at | PCTK2 | PCTAIRE protein kinase 2 | −1.48984 | 0.00019 |
| 221600_s_at | PTD015 | PTD015 protein | −1.4905 | 0.00071 |
| 221931_s_at | SEH1L | SEH1-like (S. cerevisiae) | −1.49075 | 0.000179 |
| 217836_s_at | YY1AP1 | YY1 associated protein 1 | −1.49147 | 3.85E−06 |
| 212689_s_at | JMJD1A | jumonji domain containing 1A | −1.49237 | 0.000358 |
| 204143_s_at | ENOSF1 | enolase superfamily member 1 | −1.49309 | 0.0004 |
| 207760_s_at | NCOR2 | nuclear receptor co-repressor 2 | −1.49461 | 0.001013 |
| 211953_s_at | RANBP5 | RAN binding protein 5 | −1.49478 | 3.08E−05 |
| 202583_s_at | RANBP9 | RAN binding protein 9 | −1.4971 | 0.000185 |
| 201016_at | EIF1AX | eukaryotic translation initiation factor 1A, X-linked | −1.49824 | 2.11E−05 |
| 206875_s_at | SLK | STE20-like kinase (yeast) | −1.49974 | 0.000223 |
| 218757_s_at | UPF3B | UPF3 regulator of nonsense transcripts homolog B (yeast) | −1.49992 | 2.55E−05 |
| 209377_s_at | HMGN3 | high mobility group nucleosomal binding domain 3 | −1.50027 | 0.001611 |
| 216602_s_at | FARSLA | phenylalanine-tRNA synthetase-like, alpha subunit | −1.50414 | 2.11E−07 |
| 213411_at | ADAM22 | ADAM metallopeptidase domain 22 | −1.50432 | 0.000481 |
| 202224_at | CRK | v-crk sarcoma virus CT10 oncogene homolog (avian) | −1.50461 | 0.000332 |
| 206117_at | TPM1 | tropomyosin 1 (alpha) | −1.50496 | 0.001908 |
| 221522_at | ANKRD27 | ankyrin repeat domain 27 (VPS9 domain) | −1.50633 | 0.006028 |
| 209036_s_at | MDH2 | malate dehydrogenase 2, NAD (mitochondrial) | −1.50854 | 4.08E−08 |
| 201225_s_at | SRRM1 | serine/arginine repetitive matrix 1 | −1.5101 | 0.0002 |
| 209787_s_at | HMGN4 | high mobility group nucleosomal binding domain 4 | −1.51026 | 0.000372 |
| 218946_at | HIRIP5 | HIRA interacting protein 5 | −1.51063 | 0.000587 |
| 202653_s_at | 7-Mar | membrane-associated ring finger (C3HC4) 7 | −1.51097 | 0.009913 |
| 221559_s_at | MIS12 | MIS12 homolog (yeast) | −1.51248 | 0.000328 |
| 208811_s_at | DNAJB6 | DnaJ (Hsp40) homolog, subfamily B, member 6 | −1.51341 | 0.000401 |
| 203182_s_at | SRPK2 | SFRS protein kinase 2 | −1.51506 | 5.91E−06 |
| 209131_s_at | SNAP23 | synaptosomal-associated protein, 23 kDa | −1.52073 | 0.000126 |
| 200966_x_at | ALDOA | aldolase A, fructose-bisphosphate | −1.52211 | 0.002576 |
| 203138_at | HAT1 | histone acetyltransferase 1 | −1.52247 | 3.05E−05 |
| 201435_s_at | EIF4E | eukaryotic translation initiation factor 4E | −1.52276 | 0.003818 |
| 222111_at | KIAA1164 | Family with sequence similarity 63, member B | −1.52301 | 0.000948 |
| 217858_s_at | ARMCX3 | armadillo repeat containing, X-linked 3 | −1.52363 | 0.000353 |
| 208758_at | ATIC | 5-aminoimidazole-4-carboxamide ribonucleotide | −1.52419 | 0.000468 |
| | | formyltransferase/IMP cyclohydrolase | | |
| 201689_s_at | TPD52 | tumor protein D52 | −1.52518 | 0.000567 |
| 218098_at | ARFGEF2 | ADP-ribosylation factor guanine nucleotide-exchange factor 2 | −1.52581 | 2.76E−06 |
| | | (brefeldin A-inhibited) | | |
| 204464_s_at | EDNRA | endothelin receptor type A | −1.52604 | 0.009446 |
| 203105_s_at | DNM1L | dynamin 1-like | −1.52785 | 3.42E−06 |
| 218577_at | LRRC40 | leucine rich repeat containing 40 | −1.53548 | 0.00574 |
| 221505_at | ANP32E | acidic (leucine-rich) nuclear phosphoprotein 32 family, member E | −1.53649 | 1.23E−05 |
| 212460_at | C14orf147 | chromosome 14 open reading frame 147 | −1.5378 | 0.003134 |
| 201876_at | PON2 | paraoxonase 2 | −1.53936 | 0.000298 |
| 222163_s_at | SPATA5L1 | spermatogenesis associated 5-like 1 | −1.54024 | 2.50E−07 |
| 218852_at | C14orf10 | chromosome 14 open reading frame 10 | −1.54226 | 0.001947 |
| 200037_s_at | CBX3 | chromobox homolog 3 (HP1 gamma homolog, Drosophila) /// | −1.546 | 1.40E−05 |
| | | chromobox homolog 3 (HP1 gamma homolog, Drosophila) | | |
| 202427_s_at | BRP44 | brain protein 44 | −1.54621 | 1.10E−07 |
| 212530_at | NEK7 | NIMA (never in mitosis gene a)-related kinase 7 | −1.54626 | 0.000339 |
| 215596_s_at | ZNF294 | zinc finger protein 294 | −1.54835 | 9.40E−06 |
| 209948_at | KCNMB1 | potassium large conductance calcium-activated channel, | −1.54999 | 1.24E−05 |
| | | subfamily M, beta member 1 | | |
| 216331_at | ITGA7 | integrin, alpha 7 | −1.55015 | 4.44E−05 |
| 203338_at | PPP2R5E | protein phosphatase 2, regulatory subunit B (B56), epsilon | −1.55247 | 1.52E−09 |
| | | isoform | | |
| 201017_at | EIF1AX | eukaryotic translation initiation factor 1A, X-linked | −1.55346 | 1.12E−06 |
| 203062_s_at | MDC1 | mediator of DNA damage checkpoint 1 | −1.55386 | 2.91E−08 |
| 202613_at | CTPS | CTP synthase | −1.55547 | 0.000525 |
| 201237_at | CAPZA2 | capping protein (actin filament) muscle Z-line, alpha 2 | −1.55635 | 3.38E−05 |
| 218260_at | PCIA1 | cross-immune reaction antigen PCIA1 | −1.55656 | 0.001184 |
| 201362_at | IVNS1ABP | influenza virus NS1A binding protein | −1.55796 | 0.002024 |
| 209624_s_at | MCCC2 | methylcrotonoyl-Coenzyme A carboxylase 2 (beta) | −1.55914 | 0.004633 |
| 202268_s_at | APPBP1 | amyloid beta precursor protein binding protein 1 | −1.56047 | 3.09E−08 |
| 215884_s_at | UBQLN2 | ubiquilin 2 | −1.56291 | 0.002142 |
| 221893_s_at | ADCK2 | aarF domain containing kinase 2 | −1.5636 | 2.76E−08 |
| 204094_s_at | TSC22D2 | TSC22 domain family, member 2 | −1.565 | 0.001629 |
| 212817_at | DNAJB5 | DnaJ (Hsp40) homolog, subfamily B, member 5 | −1.56551 | 1.77E−05 |
| 200906_s_at | KIAA0992 | palladin | −1.56618 | 0.001667 |
| 221068_at | ANKRD25 | ankyrin repeat domain 25 | −1.56676 | 0.000956 |
| 215088_s_at | SDHC | succinate dehydrogenase complex, subunit C, integral | −1.5678 | 1.44E−08 |
| | | membrane protein, 15 kDa | | |
| 212229_s_at | FBXO21 | F-box protein 21 | −1.5695 | 0.000279 |
| 201337_s_at | VAMP3 | vesicle-associated membrane protein 3 (cellubrevin) | −1.57096 | 0.001972 |
| 212074_at | UNC84A | unc-84 homolog A (C. elegans) | −1.57112 | 0.00317 |
| 201528_at | RPA1 | replication protein A1, 70 kDa | −1.57372 | 1.34E−06 |
| 219682_s_at | TBX3 | T-box 3 (ulnar mammary syndrome) | −1.5741 | 0.005677 |
| 34031_i_at | KRIT1 | KRIT1, ankyrin repeat containing | −1.5749 | 2.36E−05 |
| 209162_s_at | PRPF4 | PRP4 pre-mRNA processing factor 4 homolog (yeast) | −1.57834 | 0.000559 |
| 221786_at | C6orf120 | chromosome 6 open reading frame 120 | −1.57871 | 0.001361 |
| 203039_s_at | NDUFS1 | NADH dehydrogenase (ubiquinone) Fe—S protein 1, 75 kDa | −1.5794 | 1.78E−07 |
| | | (NADH-coenzyme Q reductase) | | |
| 214880_x_at | CALD1 | caldesmon 1 | −1.58024 | 0.000487 |
| 221927_s_at | ABHD11 | abhydrolase domain containing 11 | −1.58086 | 0.000304 |
| 212077_at | CALD1 | caldesmon 1 | −1.58129 | 0.001327 |
| 203743_s_at | TDG | thymine-DNA glycosylase | −1.58403 | 0.002072 |
| 209448_at | HTATIP2 | HIV-1 Tat interactive protein 2, 30 kDa | −1.58613 | 0.005876 |
| 218195_at | C6orf211 | chromosome 6 open reading frame 211 | −1.58821 | 0.00064 |
| 203738_at | FLJ11193 | hypothetical protein FLJ11193 | −1.59002 | 9.21E−07 |
| 201351_s_at | YME1L1 | YME1-like 1 (S. cerevisiae) | −1.59013 | 0.000422 |
| 203200_s_at | MTRR | 5-methyltetrahydrofolate-homocysteine methyltransferase | −1.59075 | 9.30E−05 |
| | | reductase | | |
| 213154_s_at | BICD2 | bicaudal D homolog 2 (Drosophila) | −1.59204 | 8.20E−05 |
| 202829_s_at | SYBL1 | synaptobrevin-like 1 | −1.59849 | 3.76E−08 |
| 215206_at | EXT1 | Exostoses (multiple) 1 | −1.59855 | 0.007724 |
| 208783_s_at | MCP | membrane cofactor protein (CD46, trophoblast-lymphocyte | −1.60463 | 0.000109 |
| | | cross-reactive antigen) | | |
| 212151_at | PBX1 | Pre-B-cell leukemia transcription factor 1 | −1.60465 | 0.00082 |
| 218094_s_at | C20orf35 | chromosome 20 open reading frame 35 | −1.60663 | 2.03E−05 |
| 208622_s_at | VIL2 | villin 2 (ezrin) | −1.60954 | 0.007351 |
| 204744_s_at | IARS | isoleucine-tRNA synthetase | −1.6114 | 4.89E−09 |
| 202220_at | KIAA0907 | KIAA0907 | −1.61154 | 9.40E−06 |
| 214093_s_at | FUBP1 | far upstream element (FUSE) binding protein 1 | −1.61772 | 3.92E−06 |
| 213019_at | RANBP6 | RAN binding protein 6 | −1.61928 | 3.54E−06 |
| 201946_s_at | CCT2 | chaperonin containing TCP1, subunit 2 (beta) | −1.62225 | 0.003551 |
| 213743_at | CCNT2 | cyclin T2 | −1.62252 | 8.57E−05 |
| 209357_at | CITED2 | Cbp/p300-interacting transactivator, with Glu/Asp-rich carboxy- | −1.62353 | 0.003286 |
| | | terminal domain, 2 | | |
| 209786_at | HMGN4 | high mobility group nucleosomal binding domain 4 | −1.625 | 0.000338 |
| 203824_at | TSPAN8 | tetraspanin 8 | −1.62515 | 0.001642 |
| 221776_s_at | BRD7 | bromodomain containing 7 | −1.62786 | 6.20E−05 |
| 213754_s_at | PAIP1 /// | poly(A) binding protein interacting protein 1 /// similar to poly(A) | −1.6279 | 3.04E−05 |
| | LOC388345 | binding protein interacting protein 1 isoform 1; polyadenylate | | |
| | | binding protein-interacting protein 1; PABC1-interacting protein 1 | | |
| 212437_at | CENPB | centromere protein B, 80 kDa | −1.63047 | 2.17E−05 |
| 217893_s_at | C1orf108 | chromosome 1 open reading frame 108 | −1.63229 | 0.000735 |
| 211964_at | COL4A2 | collagen, type IV, alpha 2 | −1.63301 | 0.001044 |
| 203359_s_at | MYCBP | c-myc binding protein | −1.63382 | 8.50E−07 |
| 202745_at | USP8 | Ubiquitin specific peptidase 8 | −1.63484 | 4.49E−05 |
| 208637_x_at | ACTN1 | actinin, alpha 1 | −1.63704 | 7.32E−05 |
| 213501_at | ACOX1 | acyl-Coenzyme A oxidase 1, palmitoyl | −1.63823 | 4.16E−07 |
| 204485_s_at | TOM1L1 | target of myb1-like 1 (chicken) | −1.63927 | 7.45E−09 |
| 219767_s_at | CRYZL1 | crystallin, zeta (quinone reductase)-like 1 | −1.63992 | 8.65E−05 |
| 201604_s_at | PPP1R12A | protein phosphatase 1, regulatory (inhibitor) subunit 12A | −1.64396 | 1.72E−07 |
| 208786_s_at | MAP1LC3B | microtubule-associated protein 1 light chain 3 beta | −1.64459 | 2.79E−09 |
| 212037_at | PNN | pinin, desmosome associated protein | −1.64889 | 1.37E−06 |
| 213212_x_at | LOC161527 | hypothetical protein LOC161527 | −1.6494 | 0.001281 |
| 202813_at | TARBP1 | TAR (HIV) RNA binding protein 1 | −1.65057 | 0.000869 |
| 209430_at | BTAF1 | BTAF1 RNA polymerase II, B-TFIID transcription factor- | −1.65161 | 2.59E−05 |
| | | associated, 170 kDa (Mot1 homolog, S. cerevisiae) | | |
| 217894_at | KCTD3 | potassium channel tetramerisation domain containing 3 | −1.65624 | 0.003429 |
| 218085_at | CHMP5 | chromatin modifying protein 5 | −1.65706 | 0.000348 |
| 209296_at | PPM1B | protein phosphatase 1B (formerly 2C), magnesium-dependent, | −1.65803 | 0.001619 |
| | | beta isoform | | |
| 214709_s_at | KTN1 /// PDIA6 | kinectin 1 (kinesin receptor) /// protein disulfide isomerase family | −1.66634 | 2.49E−07 |
| | | A, member 6 | | |
| 200738_s_at | PGK1 | phosphoglycerate kinase 1 | −1.66769 | 0.002726 |
| 217743_s_at | TMEM30A | transmembrane protein 30A | −1.66844 | 0.000427 |
| 203788_s_at | SEMA3C | sema domain, immunoglobulin domain (Ig), short basic domain, | −1.66918 | 0.005397 |
| | | secreted, (semaphorin) 3C | | |
| 204547_at | RAB40B | RAB40B, member RAS oncogene family | −1.6723 | 0.000196 |
| 206075_s_at | CSNK2A1 | casein kinase 2, alpha 1 polypeptide | −1.67328 | 0.000519 |
| 202582_s_at | RANBP9 | RAN binding protein 9 | −1.67723 | 0.000666 |
| 201082_s_at | DCTN1 | dynactin 1 (p150, glued homolog, Drosophila) | −1.67849 | 3.97E−05 |
| 208839_s_at | CAND1 | cullin-associated and neddylation-dissociated 1 | −1.68243 | 3.76E−07 |
| 212060_at | SR140 | U2-associated SR140 protein | −1.684 | 1.49E−05 |
| 212757_s_at | CAMK2G | calcium/calmodulin-dependent protein kinase (CaM kinase) II | −1.6843 | 4.39E−08 |
| | | gamma | | |
| 212136_at | ATP2B4 | ATPase, Ca++ transporting, plasma membrane 4 | −1.68746 | 2.44E−06 |
| 207876_s_at | FLNC | filamin C, gamma (actin binding protein 280) | −1.68761 | 2.50E−05 |
| 218932_at | C1orf181 | chromosome 1 open reading frame 181 | −1.68931 | 2.68E−06 |
| 202762_at | ROCK2 | Rho-associated, coiled-coil containing protein kinase 2 | −1.69096 | 0.000114 |
| 200910_at | CCT3 | chaperonin containing TCP1, subunit 3 (gamma) | −1.69181 | 6.06E−08 |
| 212250_at | MTDH | metadherin | −1.69266 | 0.000179 |
| 211098_x_at | TMCO1 | transmembrane and coiled-coil domains 1 | −1.69399 | 4.62E−07 |
| 214157_at | GNAS | GNAS complex locus | −1.69826 | 8.15E−07 |
| 222201_s_at | CASP8AP2 | CASP8 associated protein 2 | −1.70002 | 0.004861 |
| 200884_at | CKB | creatine kinase, brain | −1.70005 | 0.001277 |
| 214691_x_at | FAM63B | family with sequence similarity 63, member B | −1.70153 | 1.59E−05 |
| 209825_s_at | UCK2 | uridine-cytidine kinase 2 | −1.70186 | 8.26E−07 |
| 202506_at | SSFA2 | sperm specific antigen 2 | −1.70323 | 0.003386 |
| 214679_x_at | GNA11 | guanine nucleotide binding protein (G protein), alpha 11 (Gq | −1.70359 | 8.13E−09 |
| | | class) | | |
| 203226_s_at | TSPAN31 | tetraspanin 31 | −1.70566 | 3.15E−05 |
| 201366_at | ANXA7 | annexin A7 | −1.70706 | 1.27E−08 |
| 204005_s_at | PAWR | PRKC, apoptosis, WT1, regulator | −1.70834 | 2.02E−05 |
| 206104_at | ISL1 | ISL1 transcription factor, LIM/homeodomain, (islet-1) | −1.70856 | 0.000144 |
| 200638_s_at | YWHAZ | tyrosine 3-monooxygenase/tryptophan 5-monooxygenase | −1.70944 | 0.000179 |
| | | activation protein, zeta polypeptide | | |
| 219913_s_at | CRNKL1 | Crn, crooked neck-like 1 (Drosophila) | −1.70974 | 0.001138 |
| 219496_at | C2orf26 | chromosome 2 open reading frame 26 | −1.71019 | 0.000249 |
| 203925_at | GCLM | glutamate-cysteine ligase, modifier subunit | −1.71141 | 0.000626 |
| 218330_s_at | NAV2 | neuron navigator 2 | −1.71283 | 0.001506 |
| 202886_s_at | PPP2R1B | protein phosphatase 2 (formerly 2A), regulatory subunit A (PR | −1.71642 | 0.000389 |
| | | 65), beta isoform | | |
| 221311_x_at | DJ122O8.2 | hypothetical protein dJ122O8.2 | −1.72017 | 2.08E−06 |
| 211698_at | CRI1 | CREBBP/EP300 inhibitor 1 /// CREBBP/EP300 inhibitor 1 | −1.72053 | 0.000765 |
| 204489_s_at | CD44 | CD44 antigen (homing function and Indian blood group system) | −1.72232 | 0.006063 |
| 202843_at | DNAJB9 | DnaJ (Hsp40) homolog, subfamily B, member 9 | −1.72238 | 0.001296 |
| 209268_at | VPS45A | vacuolar protein sorting 45A (yeast) | −1.72261 | 1.23E−05 |
| 213111_at | PIP5K3 | phosphatidylinositol-3-phosphate/phosphatidylinositol 5-kinase, | −1.72469 | 0.001958 |
| | | type III | | |
| 218898_at | FAM57A | family with sequence similarity 57, member A | −1.72689 | 0.003163 |
| 203356_at | CAPN7 | calpain 7 | −1.72718 | 2.14E−10 |
| 201001_s_at | UBE2V1 /// Kua- | ubiquitin-conjugating enzyme E2 variant 1 /// ubiquitin- | −1.72877 | 2.27E−05 |
| | UEV | conjugating enzyme E2 variant 1 | | |
| 207169_x_at | DDR1 | discoidin domain receptor family, member 1 | −1.72882 | 2.45E−06 |
| 209120_at | NR2F2 | nuclear receptor subfamily 2, group F, member 2 | −1.72972 | 0.004022 |
| 208790_s_at | PTRF | polymerase I and transcript release factor | −1.73643 | 1.24E−05 |
| 201603_at | PPP1R12A | protein phosphatase 1, regulatory (inhibitor) subunit 12A | −1.74027 | 1.12E−06 |
| 212776_s_at | OBSL1 | obscurin-like 1 | −1.74061 | 4.52E−05 |
| 200955_at | IMMT | inner membrane protein, mitochondrial (mitofilin) | −1.74252 | 1.37E−09 |
| 201243_s_at | ATP1B1 | ATPase, Na+/K+ transporting, beta 1 polypeptide | −1.74994 | 0.004339 |
| 221935_s_at | AER61 | AER61 glycosyltransferase | −1.75053 | 4.61E−07 |
| 218706_s_at | NS3TP2 | HCV NS3-transactivated protein 2 | −1.75134 | 0.007618 |
| 207945_s_at | CSNK1D | casein kinase 1, delta | −1.75619 | 0.000322 |
| 210087_s_at | MPZL1 | myelin protein zero-like 1 | −1.76171 | 0.004219 |
| 222235_s_at | GALNACT-2 | chondroitin sulfate GalNAcT-2 | −1.76287 | 0.003646 |
| 213364_s_at | SNX1 | sorting nexin 1 | −1.76412 | 0.000258 |
| 209135_at | ASPH | aspartate beta-hydroxylase | −1.76629 | 4.18E−06 |
| 211562_s_at | LMOD1 | leiomodin 1 (smooth muscle) | −1.76712 | 3.75E−09 |
| 213128_s_at | UBE3A | ubiquitin protein ligase E3A (human papilloma virus E6- | −1.76929 | 1.18E−06 |
| | | associated protein, Angelman syndrome) | | |
| 219843_at | IPP | intracisternal A particle-promoted polypeptide | −1.77164 | 2.90E−06 |
| 203254_s_at | TLN1 | talin 1 | −1.77176 | 4.03E−06 |
| 218250_s_at | CNOT7 | CCR4-NOT transcription complex, subunit 7 | −1.77206 | 0.005648 |
| 221747_at | TNS | Tensin 1 /// Tensin 1 | −1.77233 | 3.53E−07 |
| 201957_at | PPP1R12B | protein phosphatase 1, regulatory (inhibitor) subunit 12B | −1.77349 | 5.96E−06 |
| 203250_at | RBM16 | RNA binding motif protein 16 | −1.77564 | 2.93E−06 |
| 204288_s_at | SORBS2 | sorbin and SH3 domain containing 2 | −1.77859 | 1.24E−05 |
| 201539_s_at | FHL1 | four and a half LIM domains 1 | −1.77889 | 0.002179 |
| 204427_s_at | TMED2 | transmembrane emp24 domain trafficking protein 2 | −1.78055 | 0.002846 |
| 209647_s_at | SOCS5 | suppressor of cytokine signaling 5 | −1.78614 | 0.000445 |
| 210337_s_at | ACLY | ATP citrate lyase | −1.78807 | 0.000218 |
| 204161_s_at | ENPP4 | ectonucleotide pyrophosphatase/phosphodiesterase 4 (putative | −1.79121 | 0.00034 |
| | | function) | | |
| 221493_at | TSPYL1 | TSPY-like 1 | −1.79189 | 0.003039 |
| 212582_at | OSBPL8 | oxysterol binding protein-like 8 | −1.79222 | 1.56E−09 |
| 209860_s_at | ANXA7 | annexin A7 | −1.79792 | 1.52E−06 |
| 204565_at | THEM2 | thioesterase superfamily member 2 | −1.79809 | 4.11E−05 |
| 204365_s_at | C2orf23 | chromosome 2 open reading frame 23 | −1.79817 | 4.53E−06 |
| 202825_at | SLC25A4 | solute carrier family 25 (mitochondrial carrier; adenine nucleotide | −1.79975 | 2.03E−10 |
| | | translocator), member 4 | | |
| 208716_s_at | TMCO1 | transmembrane and coiled-coil domains 1 | −1.80339 | 1.59E−10 |
| 221553_at | DKFZp564K142 | implantation-associated protein | −1.80799 | 4.17E−06 |
| 213766_x_at | GNA11 | guanine nucleotide binding protein (G protein), alpha 11 (Gq | −1.80973 | 1.62E−08 |
| | | class) | | |
| 207543_s_at | P4HA1 | procollagen-proline, 2-oxoglutarate 4-dioxygenase (proline 4- | −1.81261 | 0.000368 |
| | | hydroxylase), alpha polypeptide I | | |
| 211727_s_at | COX11 | COX11 homolog, cytochrome c oxidase assembly protein (yeast) | −1.81286 | 0.001772 |
| | | /// COX11 homolog, cytochrome c oxidase assembly protein | | |
| | | (yeast) | | |
| 201612_at | ALDH9A1 | aldehyde dehydrogenase 9 family, member A1 | −1.81643 | 9.39E−07 |
| 221090_s_at | FLJ10826 | hypothetical protein FLJ10826 | −1.81667 | 4.73E−05 |
| 218772_x_at | TMEM38B | transmembrane protein 38B | −1.82146 | 5.91E−08 |
| 206557_at | FLJ12985 | Zinc finger protein 702 | −1.82179 | 6.50E−05 |
| 214805_at | EIF4A1 | Eukaryotic translation initiation factor 4A, isoform 1 | −1.82284 | 0.005966 |
| 222266_at | C19orf2 | Chromosome 19 open reading frame 2 | −1.82923 | 0.005127 |
| 212150_at | KIAA0143 | KIAA0143 protein | −1.83969 | 0.000322 |
| 214544_s_at | SNAP23 | synaptosomal-associated protein, 23 kDa | −1.84384 | 0.000181 |
| 200670_at | XBP1 | X-box binding protein 1 | −1.85062 | 0.000241 |
| 212218_s_at | FASN | fatty acid synthase | −1.85485 | 1.40E−07 |
| 200729_s_at | ACTR2 | ARP2 actin-related protein 2 homolog (yeast) | −1.85757 | 0.000313 |
| 209648_x_at | SOCS5 | suppressor of cytokine signaling 5 | −1.85986 | 5.90E−05 |
| 201912_s_at | GSPT1 | G1 to S phase transition 1 /// G1 to S phase transition 1 | −1.86127 | 3.45E−07 |
| 207980_s_at | CITED2 | Cbp/p300-interacting transactivator, with Glu/Asp-rich carboxy- | −1.8614 | 0.003126 |
| | | terminal domain, 2 | | |
| 213746_s_at | FLNA | filamin A, alpha (actin binding protein 280) | −1.86727 | 2.24E−08 |
| 217945_at | BTBD1 | BTB (POZ) domain containing 1 | −1.87215 | 4.12E−12 |
| 201238_s_at | CAPZA2 | capping protein (actin filament) muscle Z-line, alpha 2 | −1.87301 | 5.51E−05 |
| 218185_s_at | ARMC1 | armadillo repeat containing 1 | −1.87395 | 7.31E−05 |
| 202514_at | DLG1 | discs, large homolog 1 (Drosophila) | −1.87486 | 1.15E−05 |
| 208673_s_at | SFRS3 | splicing factor, arginine/serine-rich 3 | −1.87585 | 5.52E−06 |
| 212730_at | DMN | desmuslin | −1.87693 | 3.81E−06 |
| 201304_at | NDUFA5 | NADH dehydrogenase (ubiquinone) 1 alpha subcomplex, 5, | −1.88341 | 0.003896 |
| | | 13 kDa | | |
| 203068_at | KLHL21 | kelch-like 21 (Drosophila) | −1.88484 | 1.10E−07 |
| 211930_at | HNRPA3 | heterogeneous nuclear ribonucleoprotein A3 | −1.88806 | 7.55E−11 |
| 204170_s_at | CKS2 | CDC28 protein kinase regulatory subunit 2 | −1.89169 | 5.10E−06 |
| 200887_s_at | STAT1 | signal transducer and activator of transcription 1, 91 kDa | −1.89637 | 0.004578 |
| 202770_s_at | CCNG2 | cyclin G2 | −1.89759 | 0.003851 |
| 202211_at | ARFGAP3 | ADP-ribosylation factor GTPase activating protein 3 | −1.8986 | 1.22E−05 |
| 217908_s_at | IQWD1 | IQ motif and WD repeats 1 | −1.90925 | 1.61E−09 |
| 204754_at | HLF | Hepatic leukemia factor | −1.9096 | 2.09E−07 |
| 201833_at | HDAC2 | histone deacetylase 2 | −1.91356 | 0.000305 |
| 218566_s_at | CHORDC1 | cysteine and histidine-rich domain (CHORD)-containing 1 | −1.91536 | 9.30E−05 |
| 212176_at | C6orf111 | chromosome 6 open reading frame 111 | −1.9163 | 0.000142 |
| 208910_s_at | C1QBP | complement component 1, q subcomponent binding protein | −1.91635 | 1.18E−07 |
| 213077_at | YTHDC2 | YTH domain containing 2 | −1.91787 | 0.000484 |
| 212158_at | SDC2 | syndecan 2 (heparan sulfate proteoglycan 1, cell surface- | −1.91803 | 0.000658 |
| | | associated, fibroglycan) | | |
| 209273_s_at | HBLD2 | HESB like domain containing 2 | −1.92161 | 5.72E−11 |
| 202137_s_at | ZMYND11 | zinc finger, MYND domain containing 11 | −1.92386 | 8.28E−05 |
| 212006_at | UBXD2 | UBX domain containing 2 | −1.92468 | 3.56E−06 |
| 202557_at | STCH | stress 70 protein chaperone, microsome-associated, 60 kDa | −1.92893 | 7.73E−07 |
| 209656_s_at | TMEM47 | transmembrane protein 47 | −1.93241 | 4.36E−05 |
| 202979_s_at | ZF | HCF-binding transcription factor Zhangfei | −1.93537 | 1.22E−05 |
| 201824_at | RNF14 | ring finger protein 14 | −1.9481 | 1.36E−07 |
| 203566_s_at | AGL | amylo-1,6-glucosidase, 4-alpha-glucanotransferase (glycogen | −1.95262 | 0.000102 |
| | | debranching enzyme, glycogen storage disease type III) | | |
| 203594_at | RTCD1 | RNA terminal phosphate cyclase domain 1 | −1.953 | 1.06E−10 |
| 203585_at | ZNF185 | zinc finger protein 185 (LIM domain) | −1.95895 | 2.69E−07 |
| 213222_at | PLCB1 | phospholipase C, beta 1 (phosphoinositide-specific) | −1.95947 | 0.000218 |
| 218930_s_at | FLJ11273 | hypothetical protein FLJ11273 | −1.96015 | 0.001827 |
| 212040_at | TGOLN2 | trans-golgi network protein 2 | −1.96126 | 1.90E−05 |
| 208920_at | SRI | sorcin | −1.96441 | 1.63E−06 |
| 222108_at | AMIGO2 | adhesion molecule with Ig-like domain 2 | −1.96457 | 0.004185 |
| 201862_s_at | LRRFIP1 | leucine rich repeat (in FLII) interacting protein 1 | −1.96647 | 4.29E−06 |
| 207627_s_at | TFCP2 | transcription factor CP2 | −1.96732 | 1.68E−06 |
| 218804_at | TMEM16A | transmembrane protein 16A | −1.98108 | 0.008872 |
| 209427_at | SMTN | smoothelin | −1.98146 | 1.35E−07 |
| 210298_x_at | FHL1 | four and a half LIM domains 1 | −1.98171 | 0.000162 |
| 209288_s_at | CDC42EP3 | CDC42 effector protein (Rho GTPase binding) 3 | −1.99156 | 0.000246 |
| 213251_at | — | Hypothetical LOC 441046 | −1.99278 | 6.13E−05 |
| 208927_at | SPOP | speckle-type POZ protein | −1.9942 | 8.45E−05 |
| 202478_at | TRIB2 | tribbles homolog 2 (Drosophila) | −2.00034 | 0.001647 |
| 221675_s_at | CHPT1 | choline phosphotransferase 1 | −2.00503 | 0.003529 |
| 206667_s_at | SCAMP1 | secretory carrier membrane protein 1 | −2.01151 | 1.74E−05 |
| 218186_at | RAB25 | RAB25, member RAS oncogene family | −2.01581 | 4.87E−08 |
| 204751_x_at | DSC2 | desmocollin 2 | −2.01884 | 0.006288 |
| 201667_at | GJA1 | gap junction protein, alpha 1, 43 kDa (connexin 43) | −2.02437 | 0.000112 |
| 203992_s_at | UTX | ubiquitously transcribed tetratricopeptide repeat, X chromosome | −2.02454 | 0.000375 |
| 205769_at | SLC27A2 | solute carrier family 27 (fatty acid transporter), member 2 | −2.02814 | 0.001939 |
| 203881_s_at | DMD | dystrophin (muscular dystrophy, Duchenne and Becker types) | −2.03223 | 0.001477 |
| 203403_s_at | RNF6 | ring finger protein (C3H2C3 type) 6 | −2.0334 | 3.31E−06 |
| 204979_s_at | SH3BGR | SH3 domain binding glutamic acid-rich protein | −2.03864 | 0.000533 |
| 219654_at | PTPLA | protein tyrosine phosphatase-like (proline instead of catalytic | −2.04148 | 8.48E−05 |
| | | arginine), member a | | |
| 200935_at | CALR | calreticulin | −2.04644 | 0.000706 |
| 209645_s_at | ALDH1B1 | aldehyde dehydrogenase 1 family, member B1 | −2.05037 | 4.03E−08 |
| 203803_at | PCYOX1 | prenylcysteine oxidase 1 | −2.05093 | 0.004317 |
| 206303_s_at | NUDT4 /// | nudix (nucleoside diphosphate linked moiety X)-type motif 4 /// | −2.05148 | 0.000443 |
| | NUDT4P1 | nudix (nucleoside diphosphate linked moiety X)-type motif 4 | | |
| | | pseudogene 1 | | |
| 202336_s_at | PAM | peptidylglycine alpha-amidating monooxygenase | −2.05233 | 0.001771 |
| 213156_at | — | MRNA; cDNA DKFZp586B211 (from clone DKFZp586B211) | −2.05261 | 0.000772 |
| 205361_s_at | PFDN4 | prefoldin 4 | −2.05772 | 6.67E−07 |
| 212887_at | SEC23A | Sec23 homolog A (S. cerevisiae) | −2.05799 | 0.000345 |
| 201955_at | CCNC | cyclin C | −2.05848 | 3.30E−05 |
| 204490_s_at | CD44 | CD44 antigen (homing function and Indian blood group system) | −2.05994 | 0.005855 |
| 201848_s_at | BNIP3 | BCL2/adenovirus E1B 19 kDa interacting protein 3 | −2.06496 | 0.0014 |
| 210299_s_at | FHL1 | four and a half LIM domains 1 | −2.06885 | 1.80E−06 |
| 203638_s_at | FGFR2 | fibroblast growth factor receptor 2 (bacteria-expressed kinase, | −2.0727 | 5.11E−10 |
| | | keratinocyte growth factor receptor, craniofacial dysostosis 1, | | |
| | | Crouzon syndrome, Pfeiffer syndrome, Jackson-Weiss | | |
| | | syndrome) | | |
| 219368_at | NAP1L2 | nucleosome assembly protein 1-like 2 | −2.0784 | 2.94E−07 |
| 221584_s_at | KCNMA1 | potassium large conductance calcium-activated channel, | −2.08387 | 1.64E−07 |
| | | subfamily M, alpha member 1 | | |
| 221452_s_at | TMEM14B | transmembrane protein 14B /// transmembrane protein 14B | −2.08392 | 2.50E−06 |
| 204235_s_at | GULP1 | GULP, engulfment adaptor PTB domain containing 1 | −2.0857 | 0.004907 |
| 212992_at | C14orf78 | chromosome 14 open reading frame 78 | −2.09526 | 0.003251 |
| 209274_s_at | HBLD2 | HESB like domain containing 2 | −2.10205 | 5.01E−13 |
| 212007_at | UBXD2 | UBX domain containing 2 | −2.10358 | 0.000101 |
| 202266_at | TTRAP | TRAF and TNF receptor associated protein | −2.10436 | 4.92E−08 |
| 209344_at | TPM4 | tropomyosin 4 | −2.10584 | 2.65E−05 |
| 202675_at | SDHB | succinate dehydrogenase complex, subunit B, iron sulfur (Ip) | −2.10802 | 2.03E−14 |
| 212354_at | SULF1 | sulfatase 1 | −2.1081 | 0.00877 |
| 202183_s_at | KIF22 | kinesin family member 22 | −2.11008 | 7.11E−07 |
| 219073_s_at | OSBPL10 | oxysterol binding protein-like 10 | −2.11353 | 0.000181 |
| 209016_s_at | KRT7 | keratin 7 | −2.12318 | 0.007208 |
| 203716_s_at | DPP4 | dipeptidylpeptidase 4 (CD26, adenosine deaminase complexing | −2.12655 | 0.001081 |
| | | protein 2) | | |
| 201646_at | SCARB2 | scavenger receptor class B, member 2 | −2.12747 | 0.002964 |
| 214724_at | DIXDC1 | DIX domain containing 1 | −2.13456 | 0.000322 |
| 222243_s_at | TOB2 | transducer of ERBB2, 2 | −2.13505 | 3.88E−06 |
| 204971_at | CSTA | cystatin A (stefin A) | −2.13646 | 0.003847 |
| 209157_at | DNAJA2 | DnaJ (Hsp40) homolog, subfamily A, member 2 | −2.14068 | 5.47E−05 |
| 202069_s_at | IDH3A | isocitrate dehydrogenase 3 (NAD+) alpha | −2.14873 | 5.33E−09 |
| 208993_s_at | PPIG | peptidyl-prolyl isomerase G (cyclophilin G) | −2.14951 | 0.000148 |
| 218136_s_at | SLC25A37 | solute carrier family 25, member 37 | −2.15913 | 1.01E−05 |
| 212774_at | ZNF238 | zinc finger protein 238 | −2.1619 | 0.000123 |
| 217959_s_at | TRAPPC4 | trafficking protein particle complex 4 | −2.16499 | 4.66E−08 |
| 209286_at | CDC42EP3 | CDC42 effector protein (Rho GTPase binding) 3 | −2.1723 | 8.67E−05 |
| 212568_s_at | DLAT | dihydrolipoamide S-acetyltransferase (E2 component of pyruvate | −2.17383 | 1.74E−09 |
| | | dehydrogenase complex) | | |
| 203961_at | NEBL | nebulette | −2.18678 | 0.00357 |
| 202527_s_at | SMAD4 | SMAD, mothers against DPP homolog 4 (Drosophila) | −2.18688 | 0.001536 |
| 212335_at | GNS | glucosamine (N-acetyl)-6-sulfatase (Sanfilippo disease IIID) | −2.18861 | 2.62E−06 |
| 213995_at | ATP5S | ATP synthase, H+ transporting, mitochondrial F0 complex, | −2.19923 | 5.28E−08 |
| | | subunit s (factor B) | | |
| 204753_s_at | HLF | hepatic leukemia factor | −2.20111 | 8.37E−05 |
| 204160_s_at | ENPP4 | ectonucleotide pyrophosphatase/phosphodiesterase 4 (putative | −2.20243 | 0.000231 |
| | | function) | | |
| 219250_s_at | FLRT3 | fibronectin leucine rich transmembrane protein 3 | −2.20861 | 4.06E−05 |
| 220432_s_at | CYP39A1 | cytochrome P450, family 39, subfamily A, polypeptide 1 | −2.21569 | 9.47E−05 |
| 208840_s_at | G3BP2 | Ras-GTPase activating protein SH3 domain-binding protein 2 | −2.2212 | 7.36E−06 |
| 204004_at | PAWR | PRKC, apoptosis, WT1, regulator | −2.22931 | 4.57E−05 |
| 219478_at | WFDC1 | WAP four-disulfide core domain 1 | −2.23953 | 1.89E−18 |
| 205542_at | STEAP1 | six transmembrane epithelial antigen of the prostate 1 | −2.2592 | 3.17E−07 |
| 204072_s_at | 13CDNA73 | hypothetical protein CG003 | −2.26562 | 1.26E−05 |
| 202704_at | TOB1 | transducer of ERBB2, 1 | −2.26992 | 1.80E−06 |
| 202934_at | HK2 | hexokinase 2 | −2.29007 | 3.10E−05 |
| 213246_at | C14orf109 | chromosome 14 open reading frame 109 | −2.2951 | 0.00074 |
| 208228_s_at | FGFR2 | fibroblast growth factor receptor 2 (bacteria-expressed kinase, | −2.29762 | 2.47E−07 |
| | | keratinocyte growth factor receptor, craniofacial dysostosis 1, | | |
| | | Crouzon syndrome, Pfeiffer syndrome, Jackson-Weiss | | |
| | | syndrome) | | |
| 202816_s_at | SS18 | synovial sarcoma translocation, chromosome 18 | −2.2996 | 2.89E−08 |
| 212633_at | KIAA0776 | KIAA0776 | −2.30333 | 0.000663 |
| 205525_at | CALD1 | caldesmon 1 | −2.30351 | 0.002743 |
| 214266_s_at | PDLIM7 | PDZ and LIM domain 7 (enigma) | −2.30965 | 2.48E−06 |
| 201823_s_at | RNF14 | ring finger protein 14 | −2.32047 | 1.58E−05 |
| 216607_s_at | CYP51A1 | cytochrome P450, family 51, subfamily A, polypeptide 1 | −2.34642 | 6.65E−05 |
| 211555_s_at | GUCY1B3 | guanylate cyclase 1, soluble, beta 3 | −2.35716 | 0.000136 |
| 214214_s_at | C1QBP | complement component 1, q subcomponent binding protein | −2.36295 | 5.10E−12 |
| 204237_at | GULP1 | GULP, engulfment adaptor PTB domain containing 1 | −2.36669 | 7.41E−05 |
| 208931_s_at | ILF3 | interleukin enhancer binding factor 3, 90 kDa | −2.37551 | 3.35E−05 |
| 206668_s_at | SCAMP1 | secretory carrier membrane protein 1 | −2.37772 | 1.64E−06 |
| 205225_at | ESR1 | estrogen receptor 1 | −2.3795 | 0.000411 |
| 205251_at | PER2 | period homolog 2 (Drosophila) | −2.38017 | 0.000225 |
| 203817_at | GUCY1B3 | guanylate cyclase 1, soluble, beta 3 | −2.38277 | 0.000126 |
| 212344_at | SULF1 | sulfatase 1 | −2.38583 | 0.002594 |
| 208319_s_at | RBM3 | RNA binding motif (RNP1, RRM) protein 3 | −2.38916 | 0.000154 |
| 218087_s_at | SORBS1 | sorbin and SH3 domain containing 1 | −2.42729 | 2.90E−13 |
| 212585_at | OSBPL8 | oxysterol binding protein-like 8 | −2.43814 | 1.23E−07 |
| 203953_s_at | CLDN3 | claudin 3 | −2.44418 | 8.94E−06 |
| 210653_s_at | BCKDHB | branched chain keto acid dehydrogenase E1, beta polypeptide | −2.44862 | 3.29E−08 |
| | | (maple syrup urine disease) | | |
| 202536_at | CHMP2B | chromatin modifying protein 2B | −2.44914 | 0.000131 |
| 207390_s_at | SMTN | smoothelin | −2.46248 | 1.27E−10 |
| 219304_s_at | PDGFD | platelet derived growth factor D | −2.47069 | 3.79E−06 |
| 214670_at | ZKSCAN1 | zinc finger with KRAB and SCAN domains 1 | −2.47404 | 3.81E−05 |
| 221207_s_at | NBEA | neurobeachin | −2.47879 | 0.000905 |
| 202537_s_at | CHMP2B | chromatin modifying protein 2B | −2.48851 | 4.74E−05 |
| 203710_at | ITPR1 | inositol 1,4,5-triphosphate receptor, type 1 | −2.4912 | 0.001794 |
| 203636_at | MID1 | midline 1 (Opitz/BBB syndrome) | −2.49756 | 4.20E−05 |
| 201616_s_at | CALD1 | caldesmon 1 | −2.4979 | 0.003965 |
| 201971_s_at | ATP6V1A | ATPase, H+ transporting, lysosomal 70 kDa, V1 subunit A | −2.51465 | 0.00051 |
| 201197_at | AMD1 | adenosylmethionine decarboxylase 1 | −2.53891 | 0.00024 |
| 201037_at | PFKP | phosphofructokinase, platelet | −2.541 | 0.000578 |
| 202817_s_at | SS18 | synovial sarcoma translocation, chromosome 18 | −2.54791 | 1.65E−09 |
| 220925_at | MAK10 | MAK10 homolog, amino-acid N-acetyltransferase subunit, | −2.57025 | 5.97E−12 |
| | | (S. cerevisiae) | | |
| 212897_at | CDC2L6 | cell division cycle 2-like 6 (CDK8-like) | −2.58059 | 0.004049 |
| 221561_at | SOAT1 | sterol O-acyltransferase (acyl-Coenzyme A: cholesterol | −2.58089 | 2.95E−05 |
| | | acyltransferase) 1 | | |
| 202236_s_at | SLC16A1 | solute carrier family 16 (monocarboxylic acid transporters), | −2.58226 | 5.20E−05 |
| | | member 1 | | |
| 209025_s_at | SYNCRIP | synaptotagmin binding, cytoplasmic RNA interacting protein | −2.59621 | 9.38E−05 |
| 210377_at | ACSM3 | acyl-CoA synthetase medium-chain family member 3 | −2.59741 | 0.007499 |
| 214651_s_at | HOXA9 | homeo box A9 | −2.60802 | 0.00096 |
| 205321_at | EIF2S3 | eukaryotic translation initiation factor 2, subunit 3 gamma, 52 kDa | −2.61371 | 3.53E−05 |
| 213924_at | MPPE1 | Metallophosphoesterase 1 | −2.62893 | 1.87E−15 |
| 203608_at | ALDH5A1 | aldehyde dehydrogenase 5 family, member A1 (succinate- | −2.66947 | 2.02E−18 |
| | | semialdehyde dehydrogenase) | | |
| 213979_s_at | CTBP1 | Hypothetical protein LOC285463 | −2.69443 | 0.006061 |
| 206116_s_at | TPM1 | tropomyosin 1 (alpha) | −2.73936 | 4.45E−10 |
| 217523_at | CD44 | CD44 antigen (homing function and Indian blood group system) | −2.74708 | 0.0095 |
| 201242_s_at | ATP1B1 | ATPase, Na+/K+ transporting, beta 1 polypeptide | −2.76061 | 0.000158 |
| 202435_s_at | CYP1B1 | cytochrome P450, family 1, subfamily B, polypeptide 1 | −2.77895 | 0.001214 |
| 203242_s_at | PDLIM5 | PDZ and LIM domain 5 | −2.77918 | 0.004791 |
| 209772_s_at | CD24 | CD24 antigen (small cell lung carcinoma cluster 4 antigen) | −2.78942 | 0.005268 |
| 204688_at | SGCE | sarcoglycan, epsilon | −2.81175 | 9.20E−07 |
| 201660_at | ACSL3 | Acyl-CoA synthetase long-chain family member 3 | −2.81947 | 1.28E−08 |
| 203789_s_at | SEMA3C | sema domain, immunoglobulin domain (Ig), short basic domain, | −2.82448 | 0.004059 |
| | | secreted, (semaphorin) 3C | | |
| 212993_at | — | CDNA clone IMAGE: 5311129 | −2.86622 | 1.19E−31 |
| 201617_x_at | CALD1 | caldesmon 1 | −2.9017 | 0.000303 |
| 222274_at | FLJ31568 | hypothetical protein FLJ31568 | −2.9124 | 0.004918 |
| 208651_x_at | CD24 | CD24 antigen (small cell lung carcinoma cluster 4 antigen) | −2.94355 | 0.000301 |
| 219455_at | FLJ21062 | hypothetical protein FLJ21062 | −2.95691 | 0.001741 |
| 218980_at | FHOD3 | formin homology 2 domain containing 3 | −2.96965 | 7.48E−12 |
| 211478_s_at | DPP4 | dipeptidylpeptidase 4 (CD26, adenosine deaminase complexing | −2.97007 | 0.000412 |
| | | protein 2) | | |
| 201662_s_at | ACSL3 | acyl-CoA synthetase long-chain family member 3 | −2.98243 | 7.94E−06 |
| 214608_s_at | EYA1 | eyes absent homolog 1 (Drosophila) | −3.00899 | 6.88E−06 |
| 203791_at | DMXL1 | Dmx-like 1 | −3.02935 | 7.63E−10 |
| 217744_s_at | PERP | PERP, TP53 apoptosis effector | −3.05305 | 9.22E−08 |
| 206302_s_at | NUDT4 /// | nudix (nucleoside diphosphate linked moiety X)-type motif 4 /// | −3.06411 | 2.34E−06 |
| | NUDT4P1 | nudix (nucleoside diphosphate linked moiety X)-type motif 4 | | |
| | | pseudogene 1 | | |
| 202720_at | TES | testis derived transcript (3 LIM domains) | −3.07305 | 3.41E−14 |
| 211137_s_at | ATP2C1 | ATPase, Ca++ transporting, type 2C, member 1 | −3.10689 | 3.52E−10 |
| 209728_at | HLA-DRB4 | major histocompatibility complex, class II, DR beta 4 /// major | −3.10752 | 0.001536 |
| | | histocompatibility complex, class II, DR beta 4 | | |
| 209138_x_at | IGLC2 | Immunoglobulin lambda joining 3 | −3.22782 | 0.007119 |
| 202719_s_at | TES | testis derived transcript (3 LIM domains) | −3.23629 | 1.01E−17 |
| 205433_at | BCHE | butyrylcholinesterase | −3.23751 | 7.10E−05 |
| 218532_s_at | FLJ20152 | hypothetical protein FLJ20152 | −3.31251 | 1.69E−07 |
| 202436_s_at | CYP1B1 | cytochrome P450, family 1, subfamily B, polypeptide 1 | −3.3167 | 0.000787 |
| 204734_at | KRT15 | keratin 15 | −3.36506 | 0.005787 |
| 216379_x_at | CD24 | CD24 antigen (small cell lung carcinoma cluster 4 antigen) | −3.37767 | 2.57E−05 |
| 201196_s_at | AMD1 | adenosylmethionine decarboxylase 1 | −3.38686 | 0.001787 |
| 221045_s_at | PER3 | period homolog 3 (Drosophila) | −3.39093 | 2.20E−12 |
| 204364_s_at | C2orf23 | chromosome 2 open reading frame 23 | −3.40932 | 2.60E−10 |
| 203637_s_at | MID1 | midline 1 (Opitz/BBB syndrome) | −3.47064 | 2.64E−15 |
| 201661_s_at | ACSL3 | acyl-CoA synthetase long-chain family member 3 | −3.49453 | 1.46E−10 |
| 203216_s_at | MYO6 | myosin VI | −3.4964 | 0.001331 |
| 209771_x_at | CD24 | CD24 antigen (small cell lung carcinoma cluster 4 antigen) | −3.51987 | 3.51E−06 |
| 208451_s_at | C4A /// C4B | complement component 4A /// complement component 4B /// | −3.59073 | 0.00994 |
| | | complement component 4B, telomeric | | |
| 220723_s_at | FLJ21511 | hypothetical protein FLJ21511 | −3.70963 | 0.00143 |
| 205357_s_at | AGTR1 | angiotensin II receptor, type 1 | −3.82225 | 0.001449 |
| 209424_s_at | AMACR | alpha-methylacyl-CoA racemase | −3.9266 | 0.001404 |
| 205549_at | PCP4 | Purkinje cell protein 4 | −4.03294 | 1.67E−12 |
| 212899_at | CDC2L6 | cell division cycle 2-like 6 (CDK8-like) | −4.12154 | 0.000178 |
| 221646_s_at | ZDHHC11 | zinc finger, DHHC-type containing 11 | −4.12614 | 2.32E−06 |
| 217022_s_at | IGHA1 /// IGHA2 | immunoglobulin heavy constant alpha 1 /// immunoglobulin | −4.29503 | 0.00533 |
| | /// MGC27165 | heavy constant alpha 2 (A2m marker) /// hypothetical protein | | |
| | | MGC27165 | | |
| 220724_at | FLJ21511 | hypothetical protein FLJ21511 | −4.31938 | 0.001539 |
| 212768_s_at | OLFM4 | olfactomedin 4 | −4.56332 | 0.003498 |
| 266_s_at | CD24 | CD24 antigen (small cell lung carcinoma cluster 4 antigen) | −4.7867 | 1.29E−06 |
| 202437_s_at | CYP1B1 | cytochrome P450, family 1, subfamily B, polypeptide 1 | −4.90567 | 1.71E−05 |
| 202888_s_at | ANPEP | alanyl (membrane) aminopeptidase (aminopeptidase N, | −4.94836 | 0.008399 |
| | | aminopeptidase M, microsomal aminopeptidase, CD13, p150) | | |
| 202018_s_at | LTF | lactotransferrin | −6.21575 | 0.004113 |
| 208650_s_at | CD24 | CD24 antigen (small cell lung carcinoma cluster 4 antigen) | −6.91087 | 2.02E−08 |
| 203815_at | GSTT1 | glutathione S-transferase theta 1 | −7.02985 | 0.002025 |

TABLE 40
dataset 3 (tumor)

| | | | log base | |
|---|---|---|---|---|
| | | | 2 fold | |
| uniqueID | Gene Symbol | Gene Title | change | p value |
| 219288_at | C3orf14 | chromosome 3 open reading frame 14 | 4.067066 | 6.30E−06 |
| 202218_s_at | FADS2 | fatty acid desaturase 2 | 3.023027 | 1.75E−05 |
| 211110_s_at | AR | androgen receptor (dihydrotestosterone receptor; testicular | 2.709222 | 5.68E−07 |
| | | feminization; spinal and bulbar muscular atrophy; Kennedy disease) | | |
| 203789_s_at | SEMA3C | sema domain, immunoglobulin domain (Ig), short basic domain, | 2.656496 | 0.001935 |
| | | secreted, (semaphorin) 3C | | |
| 212741_at | MAOA | monoamine oxidase A | 2.317777 | 0.008391 |
| 214414_x_at | HBA2 | hemoglobin, alpha 2 /// hemoglobin, alpha 2 | 2.213076 | 0.004086 |
| 211621_at | AR | androgen receptor (dihydrotestosterone receptor; testicular | 2.148523 | 4.90E−07 |
| | | feminization; spinal and bulbar muscular atrophy; Kennedy disease) | | |
| | | /// androgen receptor (dihydrotestosterone receptor; testicular | | |
| | | feminization; spinal and bulbar muscular atrophy; Kennedy disease) | | |
| 203788_s_at | SEMA3C | sema domain, immunoglobulin domain (Ig), short basic domain, | 1.986218 | 0.001431 |
| | | secreted, (semaphorin) 3C | | |
| 219682_s_at | TBX3 | T-box 3 (ulnar mammary syndrome) | 1.913071 | 0.001209 |
| 211745_x_at | HBA1 /// HBA2 | hemoglobin, alpha 1 /// hemoglobin, alpha 1 /// hemoglobin, alpha 2 | 1.911863 | 0.006057 |
| | | /// hemoglobin, alpha 2 | | |
| 205321_at | EIF2S3 | eukaryotic translation initiation factor 2, subunit 3 gamma, 52 kDa | 1.881613 | 0.009788 |
| 213506_at | F2RL1 | coagulation factor II (thrombin) receptor-like 1 | 1.834623 | 0.006611 |
| 215201_at | REPS1 | RALBP1 associated Eps domain containing 1 | 1.834164 | 7.14E−06 |
| 209656_s_at | TMEM47 | transmembrane protein 47 | 1.820966 | 0.007911 |
| 205443_at | SNAPC1 | small nuclear RNA activating complex, polypeptide 1, 43 kDa | 1.819912 | 0.005134 |
| 202478_at | TRIB2 | tribbles homolog 2 (Drosophila) | 1.816391 | 0.00647 |
| 204049_s_at | PHACTR2 | phosphatase and actin regulator 2 | 1.769946 | 0.006535 |
| 207362_at | SLC30A4 | solute carrier family 30 (zinc transporter), member 4 | 1.757568 | 2.78E−05 |
| 214850_at | LOC153561 | hypothetical protein LOC153561 | 1.741596 | 0.004555 |
| 209585_s_at | MINPP1 | multiple inositol polyphosphate histidine phosphatase, 1 | 1.732343 | 0.001992 |
| 201362_at | IVNS1ABP | influenza virus NS1A binding protein | 1.716973 | 0.000756 |
| 204364_s_at | C2orf23 | chromosome 2 open reading frame 23 | 1.710403 | 0.002502 |
| 209127_s_at | SART3 | squamous cell carcinoma antigen recognised by T cells 3 | 1.7036 | 1.78E−06 |
| 208835_s_at | CROP | cisplatin resistance-associated overexpressed protein | 1.702575 | 1.70E−05 |
| 208993_s_at | PPIG | peptidyl-prolyl isomerase G (cyclophilin G) | 1.702491 | 0.000712 |
| 206557_at | FLJ12985 | Zinc finger protein 702 | 1.7008 | 2.27E−05 |
| 220342_x_at | C1orf22 | chromosome 1 open reading frame 22 | 1.683053 | 0.007215 |
| 203824_at | TSPAN8 | tetraspanin 8 | 1.678219 | 0.006105 |
| 221345_at | GPR43 | G protein-coupled receptor 43 | 1.671761 | 0.000192 |
| 204018_x_at | HBA1 /// HBA2 | hemoglobin, alpha 1 /// hemoglobin, alpha 1 /// hemoglobin, alpha 2 | 1.662531 | 0.009874 |
| | | /// hemoglobin, alpha 2 | | |
| 212585_at | OSBPL8 | oxysterol binding protein-like 8 | 1.643106 | 0.002659 |
| 203060_s_at | PAPSS2 | 3′-phosphoadenosine 5′-phosphosulfate synthase 2 | 1.641464 | 0.006984 |
| 211272_s_at | DGKA | diacylglycerol kinase, alpha 80 kDa | 1.597754 | 0.00979 |
| 219304_s_at | PDGFD | platelet derived growth factor D | 1.554898 | 4.82E−05 |
| 217492_s_at | PTEN | phosphatase and tensin homolog (mutated in multiple advanced | 1.553955 | 0.004398 |
| | | cancers 1) | | |
| 204750_s_at | DSC2 | desmocollin 2 | 1.537239 | 0.009667 |
| 53071_s_at | FLJ22222 | hypothetical protein FLJ22222 | 1.535296 | 0.00038 |
| 214157_at | GNAS | GNAS complex locus | 1.528672 | 7.49E−05 |
| 218353_at | RGS5 | regulator of G-protein signalling 5 | 1.520824 | 0.00738 |
| 202311_s_at | COL1A1 | collagen, type I, alpha 1 | 1.510418 | 1.44E−05 |
| 202183_s_at | KIF22 | kinesin family member 22 | 1.505335 | 0.00015 |
| 211913_s_at | MERTK | c-mer proto-oncogene tyrosine kinase /// c-mer proto-oncogene | 1.50369 | 0.001928 |
| | | tyrosine kinase | | |
| 204053_x_at | PTEN | phosphatase and tensin homolog (mutated in multiple advanced | 1.470488 | 0.007709 |
| | | cancers 1) | | |
| 214214_s_at | C1QBP | complement component 1, q subcomponent binding protein | 1.441292 | 0.000129 |
| 219211_at | USP18 | ubiquitin specific peptidase 18 | 1.438648 | 0.000229 |
| 218458_at | GCL | germ cell-less homolog 1 (Drosophila) | 1.424143 | 0.001795 |
| 221509_at | DENR | density-regulated protein | 1.419064 | 0.000169 |
| 200069_at | SART3 | squamous cell carcinoma antigen recognised by T cells 3 /// | 1.410431 | 0.003193 |
| | | squamous cell carcinoma antigen recognised by T cells 3 | | |
| 210469_at | DLG5 | discs, large homolog 5 (Drosophila) | 1.393985 | 0.00705 |
| 205449_at | SAC3D1 | SAC3 domain containing 1 | 1.382232 | 0.000971 |
| 212582_at | OSBPL8 | oxysterol binding protein-like 8 | 1.380995 | 0.000279 |
| 218195_at | C6orf211 | chromosome 6 open reading frame 211 | 1.376092 | 0.001955 |
| 207627_s_at | TFCP2 | transcription factor CP2 | 1.373726 | 0.000922 |
| 219843_at | IPP | intracisternal A particle-promoted polypeptide | 1.361731 | 2.43E−05 |
| 203909_at | SLC9A6 | solute carrier family 9 (sodium/hydrogen exchanger), member 6 | 1.360926 | 0.00533 |
| 222163_s_at | SPATA5L1 | spermatogenesis associated 5-like 1 | 1.358789 | 0.000314 |
| 203608_at | ALDH5A1 | aldehyde dehydrogenase 5 family, member A1 (succinate- | 1.349743 | 0.000693 |
| | | semialdehyde dehydrogenase) | | |
| 203202_at | HRB2 | HIV-1 rev binding protein 2 | 1.349411 | 0.006111 |
| 202745_at | USP8 | Ubiquitin specific peptidase 8 | 1.343082 | 0.001595 |
| 212629_s_at | PKN2 | protein kinase N2 | 1.339101 | 0.003925 |
| 213694_at | RSBN1 | round spermatid basic protein 1 | 1.333251 | 0.001307 |
| 218519_at | SLC35A5 | solute carrier family 35, member A5 | 1.323255 | 0.0015 |
| 202817_s_at | SS18 | synovial sarcoma translocation, chromosome 18 | 1.321138 | 0.005407 |
| 217761_at | MTCBP-1 | membrane-type 1 matrix metalloproteinase cytoplasmic tail binding | 1.314466 | 1.75E−05 |
| | | protein-1 | | |
| 203637_s_at | MID1 | midline 1 (Opitz/BBB syndrome) | 1.296376 | 0.002815 |
| 203359_s_at | MYCBP | c-myc binding protein | 1.295069 | 4.32E−05 |
| 208859_s_at | ATRX | alpha thalassemia/mental retardation syndrome X-linked (RAD54 | 1.293808 | 0.006028 |
| | | homolog, S. cerevisiae) | | |
| 202173_s_at | ZNF161 | zinc finger protein 161 | 1.289034 | 0.002264 |
| 209003_at | SLC25A11 | solute carrier family 25 (mitochondrial carrier; oxoglutarate carrier), | 1.284626 | 0.000354 |
| | | member 11 | | |
| 216969_s_at | KIF22 | kinesin family member 22 | 1.27714 | 1.47E−05 |
| 201569_s_at | SAMM50 | sorting and assembly machinery component 50 homolog (S. cerevisiae) | 1.27639 | 0.000475 |
| 202330_s_at | UNG | uracil-DNA glycosylase | 1.274497 | 3.36E−06 |
| 201681_s_at | DLG5 | discs, large homolog 5 (Drosophila) | 1.262576 | 0.005287 |
| 216109_at | THRAP2 | Thyroid hormone receptor associated protein 2 | 1.259676 | 1.58E−07 |
| 213046_at | PABPN1 | poly(A) binding protein, nuclear 1 | 1.258485 | 0.005104 |
| 201528_at | RPA1 | replication protein A1, 70 kDa | 1.257077 | 0.000305 |
| 221559_s_at | MIS12 | MIS12 homolog (yeast) | 1.251199 | 0.003955 |
| 215032_at | RREB1 | ras responsive element binding protein 1 | 1.248204 | 3.25E−05 |
| 212729_at | DLG3 | discs, large homolog 3 (neuroendocrine-dlg, Drosophila) | 1.24382 | 0.008001 |
| 214442_s_at | PIAS2 | protein inhibitor of activated STAT, 2 | 1.221879 | 0.00117 |
| 219083_at | SHQ1 | SHQ1 homolog (S. cerevisiae) | 1.216521 | 4.88E−05 |
| 205760_s_at | OGG1 | 8-oxoguanine DNA glycosylase | 1.210227 | 0.00691 |
| 214352_s_at | KRAS | v-Ki-ras2 Kirsten rat sarcoma viral oncogene homolog | 1.208634 | 0.008333 |
| 201613_s_at | AP1G2 | adaptor-related protein complex 1, gamma 2 subunit | 1.207769 | 0.004899 |
| 204020_at | PURA | purine-rich element binding protein A | 1.206769 | 0.009195 |
| 219038_at | MORC4 | MORC family CW-type zinc finger 4 | 1.202674 | 0.00012 |
| 219913_s_at | CRNKL1 | Crn, crooked neck-like 1 (Drosophila) | 1.202252 | 0.002473 |
| 205120_s_at | SGCB | sarcoglycan, beta (43 kDa dystrophin-associated glycoprotein) | 1.202214 | 0.002981 |
| 213743_at | CCNT2 | cyclin T2 | 1.194943 | 0.003609 |
| 206272_at | SPHAR | S-phase response (cyclin-related) | 1.191701 | 0.0085 |
| 209865_at | SLC35A3 | solute carrier family 35 (UDP-N-acetylglucosamine (UDP-GlcNAc) | 1.190514 | 0.000377 |
| | | transporter), member A3 | | |
| 202883_s_at | PPP2R1B | protein phosphatase 2 (formerly 2A), regulatory subunit A (PR 65), | 1.182853 | 0.003062 |
| | | beta isoform | | |
| 210544_s_at | ALDH3A2 | aldehyde dehydrogenase 3 family, member A2 | 1.177396 | 0.000185 |
| 211074_at | FOLR1 | folate receptor 1 (adult) /// folate receptor 1 (adult) | 1.169878 | 0.009522 |
| 34031_i_at | KRIT1 | KRIT1, ankyrin repeat containing | 1.168183 | 0.005165 |
| 208910_s_at | C1QBP | complement component 1, q subcomponent binding protein | 1.167846 | 0.001856 |
| 217787_s_at | GALNT2 | UDP-N-acetyl-alpha-D-galactosamine:polypeptide N- | 1.161249 | 0.007012 |
| | | acetylgalactosaminyltransferase 2 (GalNAc-T2) | | |
| 203878_s_at | MMP11 | matrix metallopeptidase 11 (stromelysin 3) | 1.161194 | 2.51E−05 |
| 209023_s_at | STAG2 | stromal antigen 2 | 1.161174 | 0.000178 |
| 213761_at | MDM1 | Mdm4, transformed 3T3 cell double minute 1, p53 binding protein | 1.159253 | 0.003928 |
| | | (mouse) | | |
| 202730_s_at | PDCD4 | programmed cell death 4 (neoplastic transformation inhibitor) | 1.145461 | 0.000237 |
| 212944_at | MRPS6 | Mitochondrial ribosomal protein S6 | 1.140881 | 0.005876 |
| 214724_at | DIXDC1 | DIX domain containing 1 | 1.140197 | 0.009185 |
| 203338_at | PPP2R5E | protein phosphatase 2, regulatory subunit B (B56), epsilon isoform | 1.13641 | 0.00013 |
| 216850_at | SNRPN | small nuclear ribonucleoprotein polypeptide N | 1.133063 | 1.17E−06 |
| 214093_s_at | FUBP1 | far upstream element (FUSE) binding protein 1 | 1.130455 | 0.008355 |
| 209128_s_at | SART3 | squamous cell carcinoma antigen recognised by T cells 3 | 1.129928 | 0.000888 |
| 204491_at | PDE4D | Phosphodiesterase 4D, cAMP-specific (phosphodiesterase E3 dunce | 1.126964 | 0.008866 |
| | | homolog, Drosophila) | | |
| 208839_s_at | CAND1 | cullin-associated and neddylation-dissociated 1 | 1.125623 | 0.007541 |
| 213248_at | LOC221362 | hypothetical protein LOC221362 | 1.120646 | 0.001553 |
| 204711_at | KIAA0753 | KIAA0753 gene product | 1.110681 | 0.002072 |
| 217988_at | CCNB1IP1 | cyclin B1 interacting protein 1 | 1.10701 | 0.006957 |
| 201767_s_at | ELAC2 | elaC homolog 2 (E. coli) | 1.104187 | 0.000255 |
| 209053_s_at | WHSC1 | Wolf-Hirschhorn syndrome candidate 1 | 1.102645 | 0.00681 |
| 203200_s_at | MTRR | 5-methyltetrahydrofolate-homocysteine methyltransferase reductase | 1.09792 | 0.007408 |
| 212912_at | RPS6KA2 | ribosomal protein S6 kinase, 90 kDa, polypeptide 2 | 1.096254 | 0.006383 |
| 214855_s_at | GARNL1 | GTPase activating Rap/RanGAP domain-like 1 | 1.095963 | 0.008766 |
| 206668_s_at | SCAMP1 | secretory carrier membrane protein 1 | 1.091803 | 0.004723 |
| 201845_s_at | RYBP | RING1 and YY1 binding protein | 1.090844 | 0.00079 |
| 213348_at | CDKN1C | Cyclin-dependent kinase inhibitor 1C (p57, Kip2) | 1.089622 | 0.006002 |
| 202762_at | ROCK2 | Rho-associated, coiled-coil containing protein kinase 2 | 1.088998 | 0.003457 |
| 206770_s_at | SLC35A3 | solute carrier family 35 (UDP-N-acetylglucosamine (UDP-GlcNAc) | 1.084499 | 0.001958 |
| | | transporter), member A3 | | |
| 201917_s_at | SLC25A36 | solute carrier family 25, member 36 | 1.083744 | 0.003427 |
| 222258_s_at | SH3BP4 | SH3-domain binding protein 4 | 1.082956 | 0.00385 |
| 215160_x_at | MGC72104 | Chromosome 20 open reading frame 80 /// LOC441444 | 1.078574 | 0.00419 |
| 206723_s_at | EDG4 | endothelial differentiation, lysophosphatidic acid G-protein-coupled | 1.064646 | 0.000948 |
| | | receptor, 4 | | |
| 205198_s_at | ATP7A | ATPase, Cu++ transporting, alpha polypeptide (Menkes syndrome) | 1.05613 | 0.004607 |
| 203531_at | CUL5 | cullin 5 | 1.051908 | 0.005268 |
| 213913_s_at | KIAA0984 | KIAA0984 protein | 1.051798 | 0.002599 |
| 219571_s_at | ZNF12 | zinc finger protein 12 | 1.051784 | 0.003629 |
| 219007_at | NUP43 | nucleoporin 43 kDa | 1.041794 | 6.98E−05 |
| 219646_at | FLJ20186 | hypothetical protein FLJ20186 | 1.038638 | 3.00E−05 |
| 220399_at | FLJ22639 | hypothetical protein FLJ22639 | 1.036015 | 0.003096 |
| 218242_s_at | SUV420H1 | suppressor of variegation 4-20 homolog 1 (Drosophila) | 1.034115 | 0.000924 |
| 203228_at | PAFAH1B3 | platelet-activating factor acetylhydrolase, isoform Ib, gamma subunit | 1.030474 | 0.004992 |
| | | 29 kDa | | |
| 202558_s_at | STCH | stress 70 protein chaperone, microsome-associated, 60 kDa | 1.028977 | 0.000833 |
| 219327_s_at | GPRC5C | G protein-coupled receptor, family C, group 5, member C | 1.020353 | 0.002001 |
| 201225_s_at | SRRM1 | serine/arginine repetitive matrix 1 | 1.017392 | 0.000605 |
| 219489_s_at | NXN | nucleoredoxin | 1.015749 | 0.006265 |
| 204398_s_at | EML2 | echinoderm microtubule associated protein like 2 | 1.009072 | 0.001144 |
| 220329_s_at | C6orf96 | chromosome 6 open reading frame 96 | 1.007299 | 1.80E−05 |
| 219534_x_at | CDKN1C | cyclin-dependent kinase inhibitor 1C (p57, Kip2) | 1.002459 | 0.002548 |
| 213803_at | KPNB1 | Karyopherin (importin) beta 1 | 0.999436 | 0.006392 |
| 209158_s_at | PSCD2 | pleckstrin homology, Sec7 and coiled-coil domains 2 (cytohesin-2) | 0.995643 | 0.00734 |
| 203203_s_at | HRB2 | HIV-1 rev binding protein 2 | 0.992823 | 0.008837 |
| 215392_at | USP3 | Ubiquitin specific peptidase 3 | 0.989815 | 0.002867 |
| 220282_at | RIC3 | resistance to inhibitors of cholinesterase 3 homolog (C. elegans) | 0.988646 | 0.001173 |
| 204524_at | PDPK1 | 3-phosphoinositide dependent protein kinase-1 | 0.975606 | 0.009716 |
| 203538_at | CAMLG | calcium modulating ligand | 0.975309 | 0.006403 |
| 206503_x_at | PML | promyelocytic leukemia | 0.971696 | 0.00274 |
| 203266_s_at | MAP2K4 | mitogen-activated protein kinase kinase 4 | 0.965321 | 0.003719 |
| 218197_s_at | OXR1 | oxidation resistance 1 | 0.959707 | 0.001517 |
| 202560_s_at | C1orf77 | chromosome 1 open reading frame 77 | 0.954659 | 0.008047 |
| 213075_at | OLFML2A | olfactomedin-like 2A | 0.940105 | 0.003585 |
| 205080_at | RARB | retinoic acid receptor, beta | 0.9387 | 0.001995 |
| 212571_at | CHD8 | chromodomain helicase DNA binding protein 8 | 0.937953 | 0.006539 |
| 218009_s_at | PRC1 | protein regulator of cytokinesis 1 | 0.936341 | 0.00051 |
| 202535_at | FADD | Fas (TNFRSF6)-associated via death domain | 0.925643 | 0.003455 |
| 208753_s_at | NAP1L1 | nucleosome assembly protein 1-like 1 | 0.924593 | 0.008574 |
| 219289_at | FLJ20718 | hypothetical protein FLJ20718 | 0.918072 | 0.00231 |
| 202502_at | ACADM | acyl-Coenzyme A dehydrogenase, C-4 to C-12 straight chain | 0.91534 | 0.009955 |
| 213024_at | TMF1 | TATA element modulatory factor 1 | 0.914863 | 0.006921 |
| 222014_x_at | MTO1 | mitochondrial translation optimization 1 homolog (S. cerevisiae) | 0.911093 | 0.005143 |
| 208927_at | SPOP | speckle-type POZ protein | 0.908827 | 0.009901 |
| 202215_s_at | NFYC | nuclear transcription factor Y, gamma | 0.906604 | 0.008889 |
| 209825_s_at | UCK2 | uridine-cytidine kinase 2 | 0.898406 | 0.002707 |
| 202053_s_at | ALDH3A2 | aldehyde dehydrogenase 3 family, member A2 | 0.897673 | 0.006781 |
| 204640_s_at | SPOP | speckle-type POZ protein | 0.895822 | 0.003466 |
| 205055_at | ITGAE | integrin, alpha E (antigen CD103, human mucosal lymphocyte | 0.890654 | 0.002888 |
| | | antigen 1; alpha polypeptide) | | |
| 210156_s_at | PCMT1 | protein-L-isoaspartate (D-aspartate) O-methyltransferase | 0.886671 | 0.009755 |
| 208974_x_at | KPNB1 | karyopherin (importin) beta 1 | 0.884456 | 0.000765 |
| 219644_at | NY-REN-58 | NY-REN-58 antigen | 0.877734 | 0.005228 |
| 205565_s_at | FXN | frataxin | 0.870031 | 0.005717 |
| 218558_s_at | MRPL39 | mitochondrial ribosomal protein L39 | 0.869329 | 0.000196 |
| 201545_s_at | PABPN1 | poly(A) binding protein, nuclear 1 | 0.864923 | 0.001105 |
| 203345_s_at | MTF2 | metal response element binding transcription factor 2 | 0.863862 | 0.000394 |
| 203536_s_at | WDR39 | WD repeat domain 39 | 0.860078 | 2.49E−05 |
| 212948_at | CAMTA2 | calmodulin binding transcription activator 2 | 0.85978 | 0.00597 |
| 203464_s_at | EPN2 | epsin 2 | 0.856246 | 0.004186 |
| 204523_at | ZNF140 | zinc finger protein 140 (clone pHZ-39) | 0.855459 | 0.00521 |
| 201996_s_at | SPEN | spen homolog, transcriptional regulator (Drosophila) | 0.85276 | 0.008606 |
| 203062_s_at | MDC1 | mediator of DNA damage checkpoint 1 | 0.848795 | 0.003028 |
| 202616_s_at | MECP2 | methyl CpG binding protein 2 (Rett syndrome) | 0.845633 | 0.000942 |
| 208394_x_at | ESM1 | endothelial cell-specific molecule 1 | 0.840819 | 7.12E−07 |
| 212676_at | NF1 | Neurofibromin 1 (neurofibromatosis, von Recklinghausen disease, | 0.839477 | 0.006623 |
| | | Watson disease) | | |
| 218797_s_at | SIRT7 | sirtuin (silent mating type information regulation 2 homolog) 7 | 0.837479 | 0.008509 |
| | | (S. cerevisiae) | | |
| 221596_s_at | DKFZP564O0523 | hypothetical protein DKFZp564O0523 | 0.835732 | 0.001218 |
| 204123_at | LIG3 | ligase III, DNA, ATP-dependent | 0.834137 | 0.009136 |
| 203714_s_at | TBCE | tubulin-specific chaperone e | 0.83039 | 0.002086 |
| 201949_x_at | CAPZB | capping protein (actin filament) muscle Z-line, beta | 0.829767 | 0.002902 |
| 221068_at | ANKRD25 | ankyrin repeat domain 25 | 0.823126 | 0.006155 |
| 204690_at | STX8 | syntaxin 8 | 0.821907 | 0.001261 |
| 203497_at | PPARBP | PPAR binding protein | 0.821755 | 0.00041 |
| 213679_at | FLJ13946 | hypothetical protein FLJ13946 | 0.820334 | 5.40E−05 |
| 206088_at | — | PREDICTED: Homo sapiens similar to Hypothetical protein | 0.817328 | 0.004581 |
| | | KIAA0563 (LOC440457), mRNA | | |
| 203518_at | LYST | lysosomal trafficking regulator | 0.817247 | 0.007751 |
| 208827_at | PSMB6 | proteasome (prosome, macropain) subunit, beta type, 6 | 0.810838 | 0.002811 |
| 208336_s_at | GPSN2 | glycoprotein, synaptic 2 | 0.809057 | 0.004318 |
| 213485_s_at | ABCC10 | ATP-binding cassette, sub-family C (CFTR/MRP), member 10 | 0.806583 | 0.007075 |
| 203855_at | WDR47 | WD repeat domain 47 | 0.797869 | 0.004838 |
| 221270_s_at | QTRT1 | queuine tRNA-ribosyltransferase 1 (tRNA-guanine transglycosylase) | 0.789112 | 0.00953 |
| | | /// queuine tRNA-ribosyltransferase 1 (tRNA-guanine | | |
| | | transglycosylase) | | |
| 212209_at | THRAP2 | thyroid hormone receptor associated protein 2 | 0.788269 | 0.000482 |
| 218565_at | C9orf114 | chromosome 9 open reading frame 114 | 0.78646 | 0.004898 |
| 203577_at | GTF2H4 | general transcription factor IIH, polypeptide 4, 52 kDa | 0.784664 | 8.88E−05 |
| 215030_at | GRSF1 | G-rich RNA sequence binding factor 1 | 0.778433 | 0.005463 |
| 201125_s_at | ITGB5 | integrin, beta 5 | 0.77839 | 0.003883 |
| 207941_s_at | RNPC2 | RNA-binding region (RNP1, RRM) containing 2 | 0.777662 | 0.000741 |
| 37433_at | PIAS2 | protein inhibitor of activated STAT, 2 | 0.77751 | 0.000533 |
| 211168_s_at | RENT1 | regulator of nonsense transcripts 1 | 0.770465 | 0.00036 |
| 204906_at | RPS6KA2 | ribosomal protein S6 kinase, 90 kDa, polypeptide 2 | 0.766848 | 0.000737 |
| 212891_s_at | GADD45GIP1 | growth arrest and DNA-damage-inducible, gamma interacting protein 1 | 0.760508 | 0.00067 |
| 200035_at | DULLARD | dullard homolog (Xenopus laevis) /// dullard homolog (Xenopus | 0.759381 | 0.002966 |
| | | laevis) | | |
| 214718_at | GATAD1 | GATA zinc finger domain containing 1 | 0.754166 | 0.005482 |
| 218372_at | MED9 | mediator of RNA polymerase II transcription, subunit 9 homolog | 0.747654 | 0.005707 |
| | | (yeast) | | |
| 201782_s_at | AIP | aryl hydrocarbon receptor interacting protein | 0.747474 | 0.00872 |
| 218675_at | SLC22A17 | solute carrier family 22 (organic cation transporter), member 17 | 0.744203 | 0.004213 |
| 65133_i_at | ZNHIT4 | zinc finger, HIT type 4 | 0.741236 | 0.002655 |
| 218518_at | C5orf5 | chromosome 5 open reading frame 5 | 0.740421 | 0.004859 |
| 218392_x_at | SFXN1 | sideroflexin 1 | 0.737983 | 0.001674 |
| 201481_s_at | PYGB | phosphorylase, glycogen; brain | 0.733841 | 0.001111 |
| 212728_at | DLG3 | discs, large homolog 3 (neuroendocrine-dlg, Drosophila) | 0.730374 | 0.002208 |
| 201488_x_at | KHDRBS1 | KH domain containing, RNA binding, signal transduction associated 1 | 0.719914 | 0.004085 |
| 212417_at | SCAMP1 | secretory carrier membrane protein 1 | 0.71721 | 0.008274 |
| 218219_s_at | LANCL2 | LanC lantibiotic synthetase component C-like 2 (bacterial) | 0.713117 | 0.0038 |
| 220248_x_at | NSFL1C | NSFL1 (p97) cofactor (p47) | 0.710043 | 0.003337 |
| 220631_at | OSGEPL1 | O-sialoglycoprotein endopeptidase-like 1 | 0.704743 | 0.009513 |
| 213017_at | ABHD3 | abhydrolase domain containing 3 | 0.703784 | 0.003474 |
| 205068_s_at | ARHGAP26 | Rho GTPase activating protein 26 | 0.701478 | 0.009427 |
| 210418_s_at | IDH3B | isocitrate dehydrogenase 3 (NAD+) beta | 0.69943 | 0.001047 |
| 218388_at | PGLS | 6-phosphogluconolactonase | 0.698132 | 0.005032 |
| 210014_x_at | IDH3B | isocitrate dehydrogenase 3 (NAD+) beta | 0.69722 | 0.003215 |
| 201969_at | NASP | nuclear autoantigenic sperm protein (histone-binding) | 0.69612 | 0.000994 |
| 213637_at | — | Transcribed locus, moderately similar to XP_517655.1 PREDICTED: | 0.690229 | 0.005698 |
| | | similar to KIAA0825 protein [Pan troglodytes] | | |
| 205427_at | ZNF354A | zinc finger protein 354A | 0.687761 | 0.00025 |
| 204432_at | SOX12 | SRY (sex determining region Y)-box 12 | 0.687394 | 0.000548 |
| 206316_s_at | KNTC1 | kinetochore associated 1 | 0.686164 | 0.009326 |
| 205004_at | NKRF | NF-kappaB repressing factor | 0.684854 | 0.005933 |
| 209497_s_at | RBM30 | RNA binding motif protein 30 | 0.679273 | 0.006413 |
| 215004_s_at | SF4 | splicing factor 4 | 0.673834 | 0.000114 |
| 205300_s_at | U1SNRNPBP | U11/U12 snRNP 35K | 0.6704 | 0.008125 |
| 214917_at | PRKAA1 | protein kinase, AMP-activated, alpha 1 catalytic subunit | 0.666936 | 0.007991 |
| 219785_s_at | FBXO31 | F-box protein 31 | 0.666043 | 0.00107 |
| 202427_s_at | BRP44 | brain protein 44 | 0.660907 | 0.009453 |
| 204177_s_at | KLHL20 | kelch-like 20 (Drosophila) | 0.660288 | 0.007958 |
| 216074_x_at | KIBRA | KIBRA protein | 0.660013 | 0.004118 |
| 204773_at | IL11RA | interleukin 11 receptor, alpha | 0.658594 | 0.00299 |
| 201606_s_at | PWP1 | PWP1 homolog (S. cerevisiae) | 0.641727 | 0.001857 |
| 203150_at | RABEPK | Rab9 effector protein with kelch motifs | 0.638629 | 0.00532 |
| 212696_s_at | RNF4 | ring finger protein 4 | 0.636833 | 0.00773 |
| 213794_s_at | C14orf120 | chromosome 14 open reading frame 120 | 0.636331 | 0.000541 |
| 214268_s_at | MTMR4 | myotubularin related protein 4 | 0.629634 | 0.005597 |
| 205107_s_at | EFNA4 | ephrin-A4 | 0.619512 | 0.006088 |
| 210191_s_at | PHTF1 | putative homeodomain transcription factor 1 | 0.618191 | 0.0089 |
| 213507_s_at | KPNB1 | karyopherin (importin) beta 1 | 0.616189 | 0.003071 |
| 218830_at | RPL26L1 | ribosomal protein L26-like 1 | 0.613229 | 0.00604 |
| 217797_at | UFC1 | ubiquitin-fold modifier conjugating enzyme 1 | 0.613021 | 0.008014 |
| 221437_s_at | MRPS15 | mitochondrial ribosomal protein S15 /// mitochondrial ribosomal | 0.607719 | 0.002398 |
| | | protein S15 | | |
| 204772_s_at | TTF1 | transcription termination factor, RNA polymerase I | 0.602396 | 0.002102 |
| 202495_at | TBCC | tubulin-specific chaperone c | 0.5993 | 0.006674 |
| 217310_s_at | FOXJ3 | forkhead box J3 | 0.599148 | 0.006485 |
| 214150_x_at | ATP6V0E | ATPase, H+ transporting, lysosomal 9 kDa, V0 subunit e | 0.587886 | 0.000289 |
| 209858_x_at | MPPE1 | metallophosphoesterase 1 | 0.585534 | 0.007832 |
| 221540_x_at | GTF2H2 | general transcription factor IIH, polypeptide 2, 44 kDa | 0.577513 | 0.007629 |
| 216593_s_at | PIGC | phosphatidylinositol glycan, class C | 0.576766 | 0.007037 |
| 214706_at | ZNF200 | zinc finger protein 200 | 0.571539 | 0.009701 |
| 219939_s_at | CSDE1 | cold shock domain containing E1, RNA-binding | 0.568704 | 0.007941 |
| 200051_at | SART1 | squamous cell carcinoma antigen recognised by T cells /// squamous | 0.568389 | 0.002134 |
| | | cell carcinoma antigen recognised by T cells | | |
| 200658_s_at | PHB | prohibitin | 0.567932 | 0.007802 |
| 215191_at | FBXL11 | PRO1880 protein | 0.565823 | 0.008334 |
| 202477_s_at | TUBGCP2 | tubulin, gamma complex associated protein 2 | 0.55809 | 0.001313 |
| 222020_s_at | HNT | neurotrimin | 0.555671 | 0.004086 |
| 202233_s_at | UQCRH | ubiquinol-cytochrome c reductase hinge protein | 0.553152 | 0.006186 |
| 204473_s_at | ZNF592 | zinc finger protein 592 | 0.552533 | 0.004692 |
| 37012_at | CAPZB | capping protein (actin filament) muscle Z-line, beta | 0.550902 | 0.008339 |
| 216627_s_at | B4GALT1 | UDP-Gal:betaGlcNAc beta 1,4-galactosyltransferase, polypeptide 1 | 0.550445 | 0.006495 |
| 221079_s_at | METTL2 /// | methyltransferase like 2 /// hypothetical protein FLJ12760 | 0.45854 | 0.007287 |
| | FLJ12760 | | | |
| 215731_s_at | MPHOSPH9 | M-phase phosphoprotein 9 | 0.456224 | 0.006359 |
| 32723_at | CSTF1 | cleavage stimulation factor, 3′ pre-RNA, subunit 1, 50 kDa | 0.422148 | 0.002139 |
| 220383_at | ABCG5 | ATP-binding cassette, sub-family G (WHITE), member 5 (sterolin 1) | −0.4095 | 0.009772 |
| 214639_s_at | HOXA1 | homeo box A1 | −0.41373 | 0.006926 |
| 220849_at | FLJ22659 | hypothetical protein FLJ22659 | −0.42413 | 0.008853 |
| 217721_at | 7-Sep | Septin 7 | −0.42782 | 0.007502 |
| 216517_at | IGKC /// | immunoglobulin kappa constant /// similar to Ig kappa chain | −0.48361 | 0.004553 |
| | LOC339562 | | | |
| 210348_at | 4-Sep | septin 4 | −0.48448 | 0.009454 |
| 217654_at | CFLAR | CASP8 and FADD-like apoptosis regulator | −0.48737 | 0.001265 |
| 216611_s_at | SLC6A2 | solute carrier family 6 (neurotransmitter transporter, noradrenalin), | −0.50242 | 0.007256 |
| | | member 2 | | |
| 208038_at | IL1RL2 | interleukin 1 receptor-like 2 | −0.53185 | 0.006979 |
| 217308_at | OR1F2 | olfactory receptor, family 1, subfamily F, member 2 | −0.54767 | 0.001866 |
| 205106_at | MTCP1 | mature T-cell proliferation 1 | −0.55079 | 0.005913 |
| 204462_s_at | SLC16A2 | solute carrier family 16 (monocarboxylic acid transporters), member 2 | −0.55566 | 0.008771 |
| 214449_s_at | RHOQ | ras homolog gene family, member Q | −0.55673 | 0.001641 |
| 216582_at | LOC441135 | similar to b24o18.3 (POM121 membrane glycoprotein (rat homolog)- | −0.57481 | 0.002675 |
| | | like 2) | | |
| 207610_s_at | EMR2 | egf-like module containing, mucin-like, hormone receptor-like 2 | −0.58244 | 0.001963 |
| 212695_at | CRY2 | cryptochrome 2 (photolyase-like) | −0.5884 | 0.008257 |
| 215010_s_at | BRSK2 | BR serine/threonine kinase 2 | −0.58881 | 0.00489 |
| 207155_at | TBX5 | T-box 5 | −0.59748 | 0.007919 |
| 206287_s_at | ITIH4 | inter-alpha (globulin) inhibitor H4 (plasma Kallikrein-sensitive | −0.60732 | 0.004876 |
| | | glycoprotein) | | |
| 213767_at | KSR | kinase suppressor of ras | −0.61616 | 0.008156 |
| 206622_at | TRH | thyrotropin-releasing hormone | −0.61873 | 0.000967 |
| 206625_at | RDS | retinal degeneration, slow | −0.63735 | 0.005401 |
| 213678_at | — | Similar to expressed sequence AW125688 | −0.63869 | 0.003999 |
| 207704_s_at | GAS7 | growth arrest-specific 7 | −0.64135 | 0.003858 |
| 213874_at | SERPINA4 | serpin peptidase inhibitor, clade A (alpha-1 antiproteinase, | −0.64767 | 0.007313 |
| | | antitrypsin), member 4 | | |
| 39763_at | HPX | hemopexin | −0.65364 | 0.003226 |
| 209949_at | NCF2 | neutrophil cytosolic factor 2 (65 kDa, chronic granulomatous disease, | −0.65433 | 0.008782 |
| | | autosomal 2) | | |
| 210013_at | HPX | hemopexin | −0.66147 | 0.005326 |
| 217473_x_at | SLC11A1 | solute carrier family 11 (proton-coupled divalent metal ion | −0.66601 | 0.003933 |
| | | transporters), member 1 | | |
| 210223_s_at | MR1 | major histocompatibility complex, class I-related | −0.67059 | 0.000775 |
| 211560_s_at | ALAS2 | aminolevulinate, delta-, synthase 2 (sideroblastic/hypochromic | −0.68033 | 0.002911 |
| | | anemia) | | |
| 215633_x_at | LST1 | leukocyte specific transcript 1 | −0.68442 | 0.007471 |
| 217192_s_at | PRDM1 | PR domain containing 1, with ZNF domain | −0.68535 | 0.009277 |
| 215161_at | CAMK1G | calcium/calmodulin-dependent protein kinase IG | −0.68842 | 0.0036 |
| 207651_at | GPR171 | G protein-coupled receptor 171 | −0.68926 | 0.00458 |
| 221924_at | DKFZp761I2123 | hypothetical protein DKFZp761I2123 | −0.69088 | 0.003567 |
| 216318_at | — | — | −0.69666 | 0.008792 |
| 206823_at | L3MBTL | l(3)mbt-like (Drosophila) | −0.69879 | 0.002734 |
| 218953_s_at | MGC3265 | hypothetical protein MGC3265 | −0.69919 | 0.002271 |
| 202134_s_at | WWTR1 | WW domain containing transcription regulator 1 | −0.70487 | 0.007255 |
| 217222_at | V-set and | Similar to Ig heavy chain V-I region HG3 precursor | −0.70672 | 0.0055 |
| | immunoglobulin | | | |
| | domain | | | |
| | containing 6 | | | |
| 217388_s_at | KYNU | kynureninase (L-kynurenine hydrolase) | −0.70841 | 0.00134 |
| 216330_s_at | POU6F1 | POU domain, class 6, transcription factor 1 | −0.7115 | 0.002365 |
| 206516_at | AMH | anti-Mullerian hormone | −0.7191 | 0.006749 |
| 210452_x_at | CYP4F2 | cytochrome P450, family 4, subfamily F, polypeptide 2 | −0.72991 | 0.003914 |
| 207681_at | CXCR3 | chemokine (C—X—C motif) receptor 3 | −0.73158 | 0.00369 |
| 212974_at | DENND3 | DENN/MADD domain containing 3 | −0.73825 | 0.001496 |
| 209880_s_at | SELPLG | selectin P ligand | −0.73976 | 0.003232 |
| 221810_at | RAB15 | RAB15, member RAS onocogene family | −0.74749 | 0.001117 |
| 221372_s_at | P2RX2 | purinergic receptor P2X, ligand-gated ion channel, 2 | −0.76853 | 0.0036 |
| 210484_s_at | TNFRSF10C /// | tumor necrosis factor receptor superfamily, member 10c, decoy | −0.77259 | 0.004993 |
| | MGC31957 | without an intracellular domain /// hypothetical protein MGC31957 | | |
| 200696_s_at | GSN | gelsolin (amyloidosis, Finnish type) | −0.77416 | 0.002638 |
| 220088_at | C5R1 | complement component 5 receptor 1 (C5a ligand) | −0.78775 | 0.001603 |
| 205648_at | WNT2 | wingless-type MMTV integration site family member 2 | −0.78871 | 0.003585 |
| 208468_at | SOX21 | SRY (sex determining region Y)-box 21 | −0.79249 | 0.005172 |
| 206137_at | RIMS2 | regulating synaptic membrane exocytosis 2 | −0.79731 | 0.001321 |
| 214574_x_at | LST1 | leukocyte specific transcript 1 | −0.79983 | 0.001322 |
| 206646_at | GLI1 | glioma-associated oncogene homolog 1 (zinc finger protein) | −0.80886 | 0.00112 |
| 220279_at | TRIM17 | tripartite motif-containing 17 | −0.81268 | 0.007551 |
| 210133_at | CCL11 | chemokine (C-C motif) ligand 11 | −0.81299 | 0.002018 |
| 205268_s_at | ADD2 | adducin 2 (beta) | −0.81594 | 0.004522 |
| 210895_s_at | CD86 | CD86 antigen (CD28 antigen ligand 2, B7-2 antigen) | −0.82106 | 0.001723 |
| 204951_at | RHOH | ras homolog gene family, member H | −0.82636 | 7.79E−05 |
| 212268_at | SERPINB1 | serpin peptidase inhibitor, clade B (ovalbumin), member 1 | −0.834 | 0.009879 |
| 206448_at | ZNF365 | zinc finger protein 365 | −0.84281 | 0.00188 |
| 205469_s_at | IRF5 | interferon regulatory factor 5 | −0.8443 | 0.009574 |
| 203662_s_at | TMOD1 | tropomodulin 1 | −0.84727 | 0.003451 |
| 205419_at | EBI2 | Epstein-Barr virus induced gene 2 (lymphocyte-specific G protein- | −0.8528 | 0.006845 |
| | | coupled receptor) | | |
| 205192_at | MAP3K14 | mitogen-activated protein kinase kinase kinase 14 | −0.85896 | 0.006186 |
| 210538_s_at | BIRC3 | baculoviral IAP repeat-containing 3 | −0.86706 | 0.009389 |
| 215818_at | NUDT7 | nudix (nucleoside diphosphate linked moiety X)-type motif 7 | −0.87212 | 0.002775 |
| 214366_s_at | ALOX5 | arachidonate 5-lipoxygenase | −0.87512 | 0.005116 |
| 201506_at | TGFBI | transforming growth factor, beta-induced, 68 kDa | −0.87544 | 0.005326 |
| 213112_s_at | SQSTM1 | sequestosome 1 | −0.8809 | 0.002944 |
| 216842_x_at | RBMY1A1 /// | RNA binding motif protein, Y-linked, family 1, member A1 /// RNA | −0.88196 | 1.41E−05 |
| | RBMY2FP /// | binding motif protein, Y-linked, family 2, member F pseudogene /// | | |
| | RBMY1F /// | RNA binding motif protein, Y-linked, family 1, member F /// RNA | | |
| | RBMY1B /// | binding motif protein, Y-linked, family 1, member B /// RNA binding | | |
| | RBMY1D /// | motif protein, Y-linked, family 1, member D /// RNA binding motif | | |
| | RBMY1E /// | protein, Y-linked, family 1, member E /// RNA binding motif protein, Y- | | |
| | RBMY1J | linked, family 1, member J | | |
| 217728_at | S100A6 | S100 calcium binding protein A6 (calcyclin) | −0.88616 | 0.005685 |
| 216145_at | DGCR14 | DiGeorge syndrome critical region gene 14 | −0.90199 | 0.004762 |
| 210629_x_at | LST1 | leukocyte specific transcript 1 | −0.90262 | 0.003704 |
| 207244_x_at | CYP2A6 | cytochrome P450, family 2, subfamily A, polypeptide 6 | −0.91759 | 0.001697 |
| 203186_s_at | S100A4 | S100 calcium binding protein A4 (calcium protein, calvasculin, | −0.92164 | 0.005352 |
| | | metastasin, murine placental homolog) | | |
| 201508_at | IGFBP4 | insulin-like growth factor binding protein 4 | −0.93344 | 0.000822 |
| 221701_s_at | STRA6 | stimulated by retinoic acid gene 6 homolog (mouse) /// stimulated by | −0.95571 | 0.007419 |
| | | retinoic acid gene 6 homolog (mouse) | | |
| 214181_x_at | LST1 | leukocyte specific transcript 1 | −0.95899 | 0.007152 |
| 219424_at | EBI3 | Epstein-Barr virus induced gene 3 | −0.95925 | 0.003757 |
| 219005_at | C19orf4 | chromosome 19 open reading frame 4 | −0.96604 | 0.006075 |
| 211810_s_at | GALC | galactosylceramidase (Krabbe disease) | −0.96858 | 0.007687 |
| 36564_at | IBRDC3 | IBR domain containing 3 | −0.97168 | 0.000141 |
| 209993_at | ABCB1 | ATP-binding cassette, sub-family B (MDR/TAP), member 1 | −0.97493 | 0.008408 |
| 218611_at | IER5 | immediate early response 5 | −0.97621 | 0.008229 |
| 205098_at | CCR1 | chemokine (C-C motif) receptor 1 | −0.97682 | 0.004435 |
| 203234_at | UPP1 | uridine phosphorylase 1 | −0.98121 | 0.000269 |
| 211105_s_at | NFATC1 | nuclear factor of activated T-cells, cytoplasmic, calcineurin-dependent 1 | −1.00061 | 0.000521 |
| 210684_s_at | DLG4 | discs, large homolog 4 (Drosophila) | −1.0069 | 0.003919 |
| 214027_x_at | DES /// FAM48A | desmin /// family with sequence similarity 48, member A | −1.02341 | 0.00574 |
| 201497_x_at | MYH11 | myosin, heavy polypeptide 11, smooth muscle | −1.02689 | 3.52E−05 |
| 205214_at | STK17B | serine/threonine kinase 17b (apoptosis-inducing) | −1.02815 | 0.009876 |
| 210592_s_at | SAT | spermidine/spermine N1-acetyltransferase | −1.03283 | 0.004122 |
| 213958_at | CD6 | CD6 antigen /// CD6 antigen | −1.06547 | 0.000624 |
| 220046_s_at | CCNL1 | cyclin L1 | −1.06981 | 0.003328 |
| 205508_at | SCN1B | sodium channel, voltage-gated, type I, beta | −1.08276 | 0.000109 |
| 202180_s_at | MVP | major vault protein | −1.09416 | 0.00073 |
| 202510_s_at | TNFAIP2 | tumor necrosis factor, alpha-induced protein 2 | −1.10173 | 0.006535 |
| 213095_x_at | AIF1 | allograft inflammatory factor 1 | −1.10947 | 0.000301 |
| 219835_at | PRDM8 | PR domain containing 8 | −1.11325 | 0.001979 |
| 205619_s_at | MEOX1 | mesenchyme homeo box 1 | −1.12078 | 9.09E−05 |
| 206650_at | IQCC | IQ motif containing C | −1.12799 | 0.007027 |
| 217281_x_at | — | Isolate Rice94 immunoglobulin heavy chain variable region (IGVH) | −1.13472 | 0.009091 |
| 211230_s_at | PIK3CD | phosphoinositide-3-kinase, catalytic, delta polypeptide | −1.1555 | 0.000143 |
| 200986_at | SERPING1 | serpin peptidase inhibitor, clade G (C1 inhibitor), member 1, | −1.16673 | 0.000391 |
| | | (angioedema, hereditary) | | |
| 202994_s_at | FBLN1 | fibulin 1 | −1.16914 | 0.000487 |
| 221667_s_at | HSPB8 | heat shock 22 kDa protein 8 | −1.17063 | 0.007568 |
| 202274_at | ACTG2 | actin, gamma 2, smooth muscle, enteric | −1.17436 | 0.002746 |
| 212724_at | RND3 | Rho family GTPase 3 | −1.18511 | 0.006761 |
| 218843_at | FNDC4 | fibronectin type III domain containing 4 | −1.18555 | 0.002437 |
| 209101_at | CTGF | connective tissue growth factor | −1.19026 | 0.002353 |
| 215711_s_at | WEE1 | WEE1 homolog (S. pombe) | −1.22494 | 0.000228 |
| 202181_at | KIAA0247 | KIAA0247 | −1.2489 | 0.00011 |
| 204116_at | IL2RG | interleukin 2 receptor, gamma (severe combined immunodeficiency) | −1.2517 | 0.008187 |
| 203373_at | SOCS2 | suppressor of cytokine signaling 2 | −1.25455 | 0.003637 |
| 213395_at | MLC1 | megalencephalic leukoencephalopathy with subcortical cysts 1 | −1.25666 | 0.005142 |
| 212730_at | DMN | desmuslin | −1.25943 | 0.000799 |
| 203951_at | CNN1 | calponin 1, basic, smooth muscle | −1.25969 | 0.000102 |
| 210790_s_at | SAR1A | SAR1 gene homolog A (S. cerevisiae) | −1.29828 | 0.000448 |
| 209546_s_at | APOL1 | apolipoprotein L, 1 | −1.33091 | 0.001236 |
| 201422_at | IFI30 | interferon, gamma-inducible protein 30 | −1.35739 | 0.003532 |
| 209619_at | CD74 | CD74 antigen (invariant polypeptide of major histocompatibility | −1.36614 | 0.003943 |
| | | complex, class II antigen-associated) | | |
| 221541_at | CRISPLD2 | cysteine-rich secretory protein LCCL domain containing 2 | −1.40306 | 0.00564 |
| 204919_at | PRR4 | proline rich 4 (lacrimal) | −1.40401 | 0.007006 |
| 202995_s_at | FBLN1 | fibulin 1 | −1.40743 | 0.004504 |
| 204776_at | THBS4 | thrombospondin 4 | −1.43966 | 0.006918 |
| 204135_at | DOC1 | downregulated in ovarian cancer 1 | −1.44127 | 0.000171 |
| 202644_s_at | TNFAIP3 | tumor necrosis factor, alpha-induced protein 3 | −1.44919 | 0.004104 |
| 203185_at | RASSF2 | Ras association (RalGDS/AF-6) domain family 2 | −1.45221 | 3.42E−09 |
| 204545_at | PEX6 | peroxisomal biogenesis factor 6 | −1.46509 | 0.005744 |
| 221477_s_at | MGC5618 | hypothetical protein MGC5618 | −1.47585 | 0.006742 |
| 202238_s_at | NNMT | nicotinamide N-methyltransferase | −1.49812 | 0.007802 |
| 205549_at | PCP4 | Purkinje cell protein 4 | −1.50226 | 7.85E−06 |
| 209457_at | DUSP5 | dual specificity phosphatase 5 | −1.53004 | 0.001038 |
| 203108_at | GPRC5A | G protein-coupled receptor, family C, group 5, member A | −1.53257 | 0.005419 |
| 212444_at | GPCR5A | G protein-coupled receptor, family C, group 5, member A | −1.56764 | 0.00038 |
| 204670_x_at | HLA-DRB1 | major histocompatibility complex, class II, DR beta 1 | −1.58905 | 0.000663 |
| 209312_x_at | HLA-DRB1 | major histocompatibility complex, class II, DR beta 1 /// major | −1.59595 | 0.002828 |
| | | histocompatibility complex, class II, DR beta 1 | | |
| 201531_at | ZFP36 | zinc finger protein 36, C3H type, homolog (mouse) | −1.61074 | 0.009669 |
| 208335_s_at | FY | Duffy blood group | −1.61959 | 0.004673 |
| 221491_x_at | HLA-DRB1 | major histocompatibility complex, class II, DR beta 1 /// major | −1.64031 | 0.003173 |
| | | histocompatibility complex, class II, DR beta 1 | | |
| 204279_at | PSMB9 | proteasome (prosome, macropain) subunit, beta type, 9 (large | −1.65139 | 0.004823 |
| | | multifunctional peptidase 2) | | |
| 203233_at | IL4R | interleukin 4 receptor | −1.67613 | 2.59E−06 |
| 202643_s_at | TNFAIP3 | tumor necrosis factor, alpha-induced protein 3 | −1.68658 | 0.001339 |
| 215193_x_at | HLA-DRB1 | major histocompatibility complex, class II, DR beta 1 | −1.69989 | 0.004154 |
| 201787_at | FBLN1 | fibulin 1 | −1.70283 | 0.00182 |
| 201369_s_at | ZFP36L2 | zinc finger protein 36, C3H type-like 2 | −1.71275 | 0.006363 |
| 216984_x_at | IGLC2 | Immunoglobulin lambda joining 3 | −1.73017 | 0.000549 |
| 202222_s_at | DES | desmin | −1.734 | 7.77E−05 |
| 211663_x_at | PTGDS | prostaglandin D2 synthase 21 kDa (brain) /// prostaglandin D2 | −1.76838 | 0.004719 |
| | | synthase 21 kDa (brain) | | |
| 206170_at | ADRB2 | adrenergic, beta-2-, receptor, surface | −1.79295 | 0.001318 |
| 201170_s_at | BHLHB2 | basic helix-loop-helix domain containing, class B, 2 | −1.84842 | 0.001598 |
| 202269_x_at | GBP1 | guanylate binding protein 1, interferon-inducible, 67 kDa /// guanylate | −1.88781 | 0.008925 |
| | | binding protein 1, interferon-inducible, 67 kDa | | |
| 214768_x_at | IGKV1-5 | immunoglobulin kappa variable 1-5 | −1.8908 | 0.009944 |
| 209373_at | MALL | mal, T-cell differentiation protein-like | −1.90009 | 0.00187 |
| 202638_s_at | ICAM1 | intercellular adhesion molecule 1 (CD54), human rhinovirus receptor | −1.91201 | 6.42E−05 |
| 202637_s_at | ICAM1 | intercellular adhesion molecule 1 (CD54), human rhinovirus receptor | −1.9256 | 9.68E−07 |
| 215946_x_at | LOC91353 | similar to omega protein | −1.96254 | 0.001499 |
| 201473_at | JUNB | jun B proto-oncogene | −1.97058 | 0.002345 |
| 211748_x_at | PTGDS | prostaglandin D2 synthase 21 kDa (brain) /// prostaglandin D2 | −1.98953 | 0.00808 |
| | | synthase 21 kDa (brain) | | |
| 201858_s_at | PRG1 | proteoglycan 1, secretory granule | −2.00334 | 0.000796 |
| 216005_at | TNC | Tenascin C (hexabrachion) | −2.01072 | 0.00918 |
| 204148_s_at | ZP3 /// POMZP3 | zona pellucida glycoprotein 3 (sperm receptor) /// POM (POM121 | −2.03502 | 0.003689 |
| | | homolog, rat) and ZP3 fusion | | |
| 201169_s_at | BHLHB2 | basic helix-loop-helix domain containing, class B, 2 | −2.08597 | 0.000796 |
| 212187_x_at | PTGDS | prostaglandin D2 synthase 21 kDa (brain) | −2.10715 | 0.005754 |
| 209304_x_at | GADD45B | growth arrest and DNA-damage-inducible, beta | −2.11376 | 0.008923 |
| 201108_s_at | THBS1 | thrombospondin 1 | −2.23037 | 4.98E−05 |
| 210910_s_at | ZP3 /// POMZP3 | zona pellucida glycoprotein 3 (sperm receptor) /// POM (POM121 | −2.31279 | 0.002436 |
| | | homolog, rat) and ZP3 fusion | | |
| 202912_at | ADM | adrenomedullin | −2.33046 | 0.006029 |
| 207574_s_at | GADD45B | growth arrest and DNA-damage-inducible, beta | −2.34057 | 0.006435 |
| 207076_s_at | ASS | argininosuccinate synthetase | −2.39658 | 0.001673 |
| 217739_s_at | PBEF1 | pre-B-cell colony enhancing factor 1 | −2.40999 | 0.007052 |
| 202531_at | IRF1 | interferon regulatory factor 1 | −2.42019 | 4.74E−05 |
| 217378_x_at | LOC391427 | similar to Ig kappa chain precursor V region (orphon V108) - human | −2.61464 | 0.001859 |
| | | (fragment) | | |
| 203973_s_at | CEBPD | CCAAT/enhancer binding protein (C/EBP), delta | −2.64149 | 5.45E−05 |
| 215223_s_at | SOD2 | superoxide dismutase 2, mitochondrial | −2.65143 | 0.003682 |
| 213006_at | CEBPD | CCAAT/enhancer binding protein (C/EBP), delta | −2.75979 | 0.001222 |
| 202628_s_at | SERPINE1 | serpin peptidase inhibitor, clade E (nexin, plasminogen activator | −2.7701 | 0.007581 |
| | | inhibitor type 1), member 1 | | |
| 201109_s_at | THBS1 | thrombospondin 1 | −2.85057 | 0.000401 |
| 210297_s_at | MSMB | microseminoprotein, beta- | −2.85067 | 0.000135 |
| 201110_s_at | THBS1 | thrombospondin 1 | −2.96838 | 0.001816 |
| 214669_x_at | IGKC | Immunoglobulin kappa variable 1-5 | −3.13187 | 0.002404 |
| 218541_s_at | C8orf4 | chromosome 8 open reading frame 4 | −3.28004 | 0.002075 |
| 216598_s_at | CCL2 | chemokine (C-C motif) ligand 2 | −3.29501 | 5.76E−05 |
| 215176_x_at | — | Immunoglobulin kappa light chain variable region (IGKV gene), clone | −3.2963 | 0.00076 |
| | | 25 | | |
| 207430_s_at | MSMB | microseminoprotein, beta- | −3.43957 | 4.01E−05 |
| 221651_x_at | IGKC /// IGKV1-5 | immunoglobulin kappa constant /// immunoglobulin kappa variable 1-5 | −3.52633 | 0.005015 |
| 221671_x_at | IGKC /// IGKV1-5 | immunoglobulin kappa constant /// immunoglobulin kappa variable 1-5 | −3.66038 | 0.005256 |
| 215432_at | ACSM1 | acyl-CoA synthetase medium-chain family member 1 | −3.71175 | 0.000474 |
| 211430_s_at | IGH@ /// IGHG1 | immunoglobulin heavy locus /// immunoglobulin heavy constant | −3.95304 | 0.00512 |
| | /// IGHG2 /// | gamma 1 (G1m marker) /// immunoglobulin heavy constant gamma 2 | | |
| | IGHG3 /// IGHM | (G2m marker) /// immunoglobulin heavy constant gamma 3 (G3m | | |
| | | marker) /// immunoglobulin heavy constant mu | | |
| 33767_at | NEFH | neurofilament, heavy polypeptide 200 kDa | −4.52342 | 1.77E−06 |
| 207802_at | CRISP3 | cysteine-rich secretory protein 3 | −6.07299 | 0.000888 |

TABLE 41
dataset 4 (stroma)

| | | | log base 2 | |
|---|---|---|---|---|
| uniqueID | Gene Symbol | Gene Title | fold change | p value |
| 205040_at | ORM1 | orosomucoid 1 | 5.408043278 | 0.007056211 |
| 236448_at | UNC5A | unc-5 homolog A (C. elegans) | 5.350821795 | 6.12E−05 |
| 208016_s_at | AGTR1 | angiotensin II receptor, type 1 | 3.953246795 | 0.000141585 |
| 221424_s_at | OR51E2 | olfactory receptor, family 51, subfamily E, | 3.82178233 | 0.000430019 |
| | | member 2 /// olfactory receptor, family 51, | | |
| | | subfamily E, member 2 | | |
| 213592_at | AGTRL1 | angiotensin II receptor-like 1 | 3.767495711 | 3.26E−05 |
| 205357_s_at | AGTR1 | angiotensin II receptor, type 1 | 3.750615672 | 0.000163124 |
| 1555416_a_at | ALOX15B | arachidonate 15-lipoxygenase, type B | 3.575526833 | 0.004358667 |
| 206211_at | SELE | selectin E (endothelial adhesion molecule | 3.39360787 | 0.006890631 |
| | | 1) | | |
| 206684_s_at | ATF7 | activating transcription factor 7 | 3.320087779 | 1.79E−06 |
| 235641_at | TRIB1 | Tribbles homolog 1 (Drosophila) | 3.318113162 | 0.005632056 |
| 220625_s_at | ELF5 | E74-like factor 5 (ets domain transcription | 3.125878205 | 0.001204246 |
| | | factor) | | |
| 219857_at | C10orf81 | chromosome 10 open reading frame 81 | 3.115502694 | 9.75E−06 |
| 205266_at | LIF | leukemia inhibitory factor (cholinergic | 3.110756577 | 0.004378453 |
| | | differentiation factor) | | |
| 229538_s_at | IQGAP3 | IQ motif containing GTPase activating | 3.097266182 | 0.004293628 |
| | | protein 3 | | |
| 232482_at | OR51E2 | olfactory receptor, family 51, subfamily E, | 3.096176575 | 0.002033125 |
| | | member 2 | | |
| 205067_at | IL1B | interleukin 1, beta | 3.073256716 | 0.003250044 |
| 209301_at | CA2 | carbonic anhydrase II | 3.073255991 | 0.002947531 |
| 208394_x_at | ESM1 | endothelial cell-specific molecule 1 | 3.073153483 | 1.45E−07 |
| 222921_s_at | HEY2 | hairy/enhancer-of-split related with YRPW | 3.066580845 | 3.99E−05 |
| | | motif 2 | | |
| 242397_at | OLR1 | Oxidised low density lipoprotein (lectin-like) | 3.06060833 | 0.004180569 |
| | | receptor 1 | | |
| 206115_at | EGR3 | early growth response 3 | 3.048909413 | 0.001538893 |
| 205114_s_at | CCL3 /// CCL3L1 /// CCL3L3 /// | chemokine (C-C motif) ligand 3 /// | 2.996801973 | 0.008389193 |
| | LOC643930 | chemokine (C-C motif) ligand 3-like 1 /// | | |
| | | chemokine (C-C motif) ligand 3-like 3 /// | | |
| | | similar to Small inducible cytokine A3-like 1 | | |
| | | precursor (Tonsillar lymphocyte LD78 beta | | |
| | | protein) (LD78-beta(1-70)) (G0/G1 switch | | |
| | | regulatory protein 19-2) (G0S19-2 protein) | | |
| | | (PAT 464.2) | | |
| 236193_at | HIST1H2BC | histone 1, H2bc | 2.977198499 | 0.008783706 |
| 1554190_s_at | C10orf81 | chromosome 10 open reading frame 81 | 2.950933896 | 0.000746173 |
| 214455_at | HIST1H2BC | histone 1, H2bc | 2.90904688 | 0.003485163 |
| 239010_at | LOC440157 | Hypothetical gene supported by AK096951; | 2.888664122 | 0.004614792 |
| | | BC066547 | | |
| 237252_at | THBD | thrombomodulin | 2.804554099 | 0.00061707 |
| 206932_at | CH25H | cholesterol 25-hydroxylase | 2.779790928 | 0.006402281 |
| 1552727_s_at | ADAMTS17 | ADAM metallopeptidase with | 2.776404819 | 0.000377378 |
| | | thrombospondin type 1 motif, 17 | | |
| 203242_s_at | PDLIM5 | PDZ and LIM domain 5 | 2.731878892 | 0.001426293 |
| 220324_at | C6orf155 | chromosome 6 open reading frame 155 | 2.691954214 | 1.17E−05 |
| 39402_at | IL1B | interleukin 1, beta | 2.686868619 | 0.004590755 |
| 235545_at | — | — | 2.68595892 | 0.00206357 |
| 227804_at | TLCD1 | TLC domain containing 1 | 2.671306112 | 0.000697907 |
| 243056_at | C12orf60 | Chromosome 12 open reading frame 60 | 2.666221454 | 0.001055269 |
| 40687_at | GJA4 | gap junction protein, alpha 4, 37 kDa | 2.653372049 | 2.12E−05 |
| | | (connexin 37) | | |
| 219890_at | CLEC5A | C-type lectin domain family 5, member A | 2.628407049 | 0.001254549 |
| 229490_s_at | — | Transcribed locus, strongly similar to | 2.623064315 | 0.00811132 |
| | | NP_839943.2 IQ motif containing GTPase | | |
| | | activating protein 3 [Homo sapiens] | | |
| 212750_at | PPP1R16B | protein phosphatase 1, regulatory | 2.611897229 | 0.000135253 |
| | | (inhibitor) subunit 16B | | |
| 205249_at | EGR2 | early growth response 2 (Krox-20 homolog, | 2.603299374 | 0.001354366 |
| | | Drosophila) | | |
| 230061_at | TM4SF18 | Transmembrane 4 L six family member 18 | 2.551606422 | 1.74E−06 |
| 222911_s_at | CXorf36 | chromosome X open reading frame 36 | 2.551233729 | 0.001924769 |
| 229910_at | SHE | Src homology 2 domain containing E | 2.548126716 | 8.32E−07 |
| 209583_s_at | CD200 | CD200 molecule | 2.525272139 | 0.000550437 |
| 231223_at | CSMD1 | CUB and Sushi multiple domains 1 | 2.473288654 | 0.005192431 |
| 235750_at | C17orf65 | Chromosome 17 open reading frame 65 | 2.467600627 | 0.006408806 |
| 231517_at | ZYG11A | zyg-11 homolog A (C. elegans) | 2.446620255 | 0.005025787 |
| 216804_s_at | PDLIM5 | PDZ and LIM domain 5 | 2.446539071 | 0.002122243 |
| 232080_at | HECW2 | HECT, C2 and WW domain containing E3 | 2.445803535 | 4.58E−05 |
| | | ubiquitin protein ligase 2 | | |
| 218678_at | NES | nestin | 2.439916347 | 0.00267023 |
| 235291_s_at | FLJ32255 /// LOC648075 | hypothetical protein LOC643977 /// | 2.410385364 | 0.007162876 |
| | | hypothetical protein LOC648075 | | |
| 203438_at | STC2 | stanniocalcin 2 | 2.36979808 | 3.66E−05 |
| 211303_x_at | PSMAL | growth-inhibiting protein 26 | 2.364139189 | 0.005192806 |
| 206049_at | SELP | selectin P (granule membrane protein | 2.351503754 | 0.000594349 |
| | | 140 kDa, antigen CD62) | | |
| 206176_at | BMP6 | bone morphogenetic protein 6 | 2.329231815 | 0.003327924 |
| 231947_at | MYCT1 | myc target 1 | 2.306705309 | 3.08E−05 |
| 41577_at | PPP1R16B | protein phosphatase 1, regulatory | 2.293332964 | 0.0003191 |
| | | (inhibitor) subunit 16B | | |
| 241483_at | — | Transcribed locus | 2.26671137 | 0.000402276 |
| 239952_at | — | — | 2.256417115 | 3.46E−05 |
| 214846_s_at | ALPK3 | alpha-kinase 3 | 2.250683081 | 0.001561987 |
| 228311_at | BCL6B | B-cell CLL/lymphoma 6, member B (zinc | 2.249949236 | 0.003019864 |
| | | finger protein) | | |
| 229178_at | LOC145786 | hypothetical protein LOC145786 | 2.244661815 | 0.00373752 |
| 226145_s_at | FRAS1 | Fraser syndrome 1 | 2.24416144 | 5.99E−05 |
| 210015_s_at | MAP2 | microtubule-associated protein 2 | 2.241666989 | 0.002197453 |
| 1555923_a_at | C10orf114 | chromosome 10 open reading frame 114 | 2.240910689 | 1.94E−06 |
| 214081_at | PLXDC1 | plexin domain containing 1 | 2.234321147 | 2.36E−06 |
| 204904_at | GJA4 | gap junction protein, alpha 4, 37 kDa | 2.219637934 | 3.17E−06 |
| | | (connexin 37) | | |
| 205913_at | PLIN | perilipin | 2.219101373 | 0.005601102 |
| 215555_at | C1orf63 | Chromosome 1 open reading frame 63 | 2.205338072 | 0.008855613 |
| 205073_at | CYP2J2 | cytochrome P450, family 2, subfamily J, | 2.204036517 | 0.004751291 |
| | | polypeptide 2 | | |
| 240089_at | NFE2L3 | Nuclear factor (erythroid-derived 2)-like 3 | 2.203559301 | 0.005916554 |
| 206026_s_at | TNFAIP6 | tumor necrosis factor, alpha-induced | 2.200892582 | 0.003289689 |
| | | protein 6 | | |
| 227341_at | C10orf30 | Chromosome 10 open reading frame 30 | 2.195601781 | 0.007432883 |
| 217177_s_at | — | CDNA FLJ13658 fis, clone PLACE1011567 | 2.191421449 | 1.45E−05 |
| 230250_at | PTPRB | Protein tyrosine phosphatase, receptor | 2.191211502 | 1.41E−06 |
| | | type, B | | |
| 238823_at | FMNL3 | formin-like 3 | 2.190696486 | 0.006919197 |
| 205801_s_at | RASGRP3 | RAS guanyl releasing protein 3 (calcium | 2.183707758 | 0.006189875 |
| | | and DAG-regulated) | | |
| 206710_s_at | EPB41L3 | erythrocyte membrane protein band 4.1- | 2.17992363 | 0.0023621 |
| | | like 3 | | |
| 230836_at | ST8SIA4 | ST8 alpha-N-acetyl-neuraminide alpha-2,8- | 2.142222531 | 0.001912221 |
| | | sialyltransferase 4 | | |
| 237058_x_at | SLC6A13 | solute carrier family 6 (neurotransmitter | 2.139190582 | 0.004814636 |
| | | transporter, GABA), member 13 | | |
| 229902_at | LOC285682 | hypothetical protein LOC285682 | 2.137705143 | 0.000748764 |
| 221489_s_at | SPRY4 /// LOC653170 | sprouty homolog 4 (Drosophila) /// similar | 2.134669068 | 0.000239005 |
| | | to sprouty homolog 4 (Drosophila) | | |
| 209921_at | SLC7A11 | solute carrier family 7, (cationic amino acid | 2.107970601 | 0.007528045 |
| | | transporter, y+ system) member 11 | | |
| 203887_s_at | THBD | thrombomodulin | 2.102011798 | 0.008037746 |
| 206110_at | HIST1H3H | histone 1, H3h | 2.100939768 | 0.008715183 |
| 203549_s_at | LPL | lipoprotein lipase | 2.09910994 | 0.001479842 |
| 240231_at | AZIN1 | Antizyme inhibitor 1 | 2.097881987 | 0.003082138 |
| 234996_at | — | Transcribed locus | 2.097545955 | 2.01E−05 |
| 225369_at | ESAM | endothelial cell adhesion molecule | 2.09239267 | 0.00274129 |
| 205860_x_at | FOLH1 | folate hydrolase (prostate-specific | 2.092169821 | 0.007837654 |
| | | membrane antigen) 1 | | |
| 210692_s_at | SLC43A3 | solute carrier family 43, member 3 | 2.087785687 | 0.004259661 |
| 209543_s_at | CD34 | CD34 molecule | 2.075799608 | 0.000541045 |
| 229907_at | SLC18A2 | Solute carrier family 18 (vesicular | 2.073953417 | 0.008460448 |
| | | monoamine), member 2 | | |
| 204468_s_at | TIE1 | tyrosine kinase with immunoglobulin-like | 2.071924804 | 2.76E−05 |
| | | and EGF-like domains 1 | | |
| 1556211_a_at | LOC644480 | hypothetical protein LOC644480 | 2.070366169 | 0.000792157 |
| 226670_s_at | TOMM34 | Translocase of outer mitochondrial | 2.065625737 | 0.006524167 |
| | | membrane 34 | | |
| 229723_at | TAGAP | T-cell activation GTPase activating protein | 2.064155459 | 0.004138731 |
| 204368_at | SLCO2A1 | solute carrier organic anion transporter | 2.061221721 | 0.000369324 |
| | | family, member 2A1 | | |
| 219091_s_at | MMRN2 | multimerin 2 | 2.058472171 | 0.000731406 |
| 207220_at | ART4 | ADP-ribosyltransferase 4 (Dombrock blood | 2.043550429 | 0.002606982 |
| | | group) | | |
| 215363_x_at | FOLH1 | folate hydrolase (prostate-specific | 2.038520538 | 0.008711243 |
| | | membrane antigen) 1 | | |
| 243366_s_at | CERKL | Ceramide kinase-like | 2.037723885 | 0.003812539 |
| 227844_at | FMNL3 | formin-like 3 | 2.034317057 | 7.77E−05 |
| 227874_at | — | MRNA; cDNA DKFZp586N0121 (from | 2.027235442 | 0.00254241 |
| | | clone DKFZp586N0121) | | |
| 228342_s_at | ALPK3 | alpha-kinase 3 | 2.024804194 | 0.003777982 |
| 231478_at | — | Transcribed locus, strongly similar to | 2.024303242 | 0.000482019 |
| | | NP_000914.1 phosphodiesterase 4C, | | |
| | | cAMP-specific (phosphodiesterase E1 | | |
| | | dunce homolog, Drosophila); | | |
| | | Phosphodiesterase-4C, cAMP-specific | | |
| | | (dunce (Drosophila)-homolog; | | |
| | | phosphodiesterase 4C, cAMP-specific | | |
| | | (dunce (Drosophila)-homolog | | |
| | | phosphodiesterase E1) [Homo sapiens] | | |
| 231511_at | FRAS1 | Fraser syndrome 1 | 2.018151376 | 0.000119273 |
| 210944_s_at | CAPN3 | calpain 3, (p94) | 2.017761767 | 0.008704819 |
| 228877_at | RGL3 | ral guanine nucleotide dissociation | 2.015610153 | 0.003236909 |
| | | stimulator-like 3 | | |
| 219522_at | FJX1 | four jointed box 1 (Drosophila) | 2.013633336 | 0.003735488 |
| 222033_s_at | FLT1 | Fms-related tyrosine kinase 1 (vascular | 2.012805253 | 0.000690518 |
| | | endothelial growth factor/vascular | | |
| | | permeability factor receptor) | | |
| 226997_at | — | CDNA FLJ10196 fis, clone | 2.010527394 | 0.000152186 |
| | | HEMBA1004776 | | |
| 52255_s_at | COL5A3 | collagen, type V, alpha 3 | 2.007190519 | 2.59E−05 |
| 1552718_at | HARS2 | histidyl-tRNA synthetase 2 | 2.003451244 | 0.003120407 |
| 214746_s_at | ZNF467 | zinc finger protein 467 | 2.001380592 | 0.007094038 |
| 220146_at | TLR7 | toll-like receptor 7 | 1.998039338 | 0.004060542 |
| 227923_at | SHANK3 | SH3 and multiple ankyrin repeat domains 3 | 1.992908517 | 0.000103786 |
| 222881_at | HPSE | heparanase | 1.989284122 | 0.007602647 |
| 207496_at | MS4A2 | membrane-spanning 4-domains, subfamily | 1.986807874 | 0.001350489 |
| | | A, member 2 (Fc fragment of IgE, high | | |
| | | affinity I, receptor for; beta polypeptide) | | |
| 206331_at | CALCRL | calcitonin receptor-like | 1.981484115 | 0.001297671 |
| 236399_at | — | Homo sapiens, clone IMAGE: 3896086, | 1.974808828 | 0.002602538 |
| | | mRNA | | |
| 219918_s_at | ASPM | asp (abnormal spindle)-like, microcephaly | 1.973913241 | 0.003939892 |
| | | associated (Drosophila) | | |
| 211776_s_at | EPB41L3 | erythrocyte membrane protein band 4.1- | 1.970979426 | 0.003165623 |
| | | like 3 /// erythrocyte membrane protein | | |
| | | band 4.1-like 3 | | |
| 226311_at | — | CDNA clone IMAGE: 30924414 | 1.967579343 | 0.006294477 |
| 210176_at | TLR1 | toll-like receptor 1 | 1.964384754 | 0.004195626 |
| 219957_at | RUFY2 | RUN and FYVE domain containing 2 | 1.948843591 | 0.001848803 |
| 203304_at | BAMBI | BMP and activin membrane-bound inhibitor | 1.946464271 | 0.005179106 |
| | | homolog (Xenopus laevis) | | |
| 213559_s_at | ZNF467 | Zinc finger protein 467 | 1.942373909 | 0.005422526 |
| 226028_at | ROBO4 | roundabout homolog 4, magic roundabout | 1.941458091 | 0.00252824 |
| | | (Drosophila) | | |
| 219615_s_at | KCNK5 | potassium channel, subfamily K, member 5 | 1.940530983 | 0.004990723 |
| 235292_at | FLJ32255 /// LOC648075 | hypothetical protein LOC643977 /// | 1.938040561 | 0.004158854 |
| | | hypothetical protein LOC648075 | | |
| 241756_at | SMARCA2 | SWI/SNF related, matrix associated, actin | 1.93647688 | 0.005408277 |
| | | dependent regulator of chromatin, | | |
| | | subfamily a, member 2 | | |
| 202241_at | TRIB1 | tribbles homolog 1 (Drosophila) | 1.936257057 | 0.004371766 |
| 230563_at | RASGEF1A | RasGEF domain family, member 1A | 1.921734732 | 0.006690453 |
| 205046_at | CENPE | centromere protein E, 312 kDa | 1.918046257 | 0.005592302 |
| 223307_at | CDCA3 | cell division cycle associated 3 | 1.916531017 | 0.001109115 |
| 203934_at | KDR | kinase insert domain receptor (a type III | 1.907766489 | 3.99E−05 |
| | | receptor tyrosine kinase) | | |
| 1565150_at | — | EST from clone 76558, 5′ end | 1.905032486 | 0.002432867 |
| 227345_at | TNFRSF10D | tumor necrosis factor receptor superfamily, | 1.901970445 | 0.002916496 |
| | | member 10d, decoy with truncated death | | |
| | | domain | | |
| 205098_at | CCR1 | chemokine (C-C motif) receptor 1 | 1.901669293 | 0.005812599 |
| 204273_at | EDNRB | endothelin receptor type B | 1.901179034 | 0.00162158 |
| 230785_at | SALL3 | Sal-like 3 (Drosophila) | 1.896963858 | 0.002354771 |
| 239058_at | FOXC2 | Forkhead box C2 (MFH-1, mesenchyme | 1.895092406 | 6.43E−05 |
| | | forkhead 1) | | |
| 238178_at | — | — | 1.894806622 | 0.0005487 |
| 205152_at | SLC6A1 | solute carrier family 6 (neurotransmitter | 1.893392897 | 9.22E−05 |
| | | transporter, GABA), member 1 | | |
| 205101_at | CIITA | class II, major histocompatibility complex, | 1.892503737 | 0.00452133 |
| | | transactivator | | |
| 227647_at | KCNE3 | potassium voltage-gated channel, Isk- | 1.88812435 | 0.003399224 |
| | | related family, member 3 | | |
| 238780_s_at | — | MRNA; cDNA DKFZp779F2345 (from | 1.883444959 | 0.00360212 |
| | | clone DKFZp779F2345) | | |
| 1554549_a_at | WDR20 | WD repeat domain 20 | 1.875380459 | 0.006490618 |
| 239503_at | — | CDNA clone IMAGE: 5301910 | 1.873639172 | 0.000735343 |
| 1553453_at | ASB14 | ankyrin repeat and SOCS box-containing | 1.872950281 | 0.003105631 |
| | | 14 | | |
| 237261_at | — | — | 1.871933695 | 0.006392363 |
| 230800_at | ADCY4 | adenylate cyclase 4 | 1.865993188 | 0.000347884 |
| 214954_at | SUSD5 | sushi domain containing 5 | 1.861215043 | 0.005011815 |
| 231826_at | C20orf74 | chromosome 20 open reading frame 74 | 1.858261966 | 0.009706636 |
| 228314_at | — | — | 1.855898256 | 2.22E−05 |
| 242023_at | ABHD4 | Abhydrolase domain containing 4 | 1.854997567 | 0.00694901 |
| 236982_at | — | CDNA clone IMAGE: 5270500 | 1.850778417 | 0.004082207 |
| 236089_at | — | Transcribed locus | 1.850207722 | 0.002471833 |
| 224822_at | DLC1 | deleted in liver cancer 1 | 1.849814723 | 0.006032465 |
| 235878_at | TAF1B | TATA box binding protein (TBP)-associated | 1.841304294 | 0.003011236 |
| | | factor, RNA polymerase I, B, 63 kDa | | |
| 222885_at | EMCN | endomucin | 1.83600921 | 0.009864439 |
| 238682_at | CCDC96 | coiled-coil domain containing 96 | 1.825022912 | 0.003485967 |
| 209642_at | BUB1 | BUB1 budding uninhibited by | 1.822106848 | 0.001010334 |
| | | benzimidazoles 1 homolog (yeast) | | |
| 238733_at | CPM | Carboxypeptidase M | 1.815025789 | 0.002937529 |
| 218660_at | DYSF | dysferlin, limb girdle muscular dystrophy 2B | 1.801249757 | 0.000793204 |
| | | (autosomal recessive) | | |
| 205151_s_at | KIAA0644 | KIAA0644 gene product | 1.800883601 | 0.000710068 |
| 219026_s_at | RASAL2 | RAS protein activator like 2 | 1.797465222 | 1.14E−06 |
| 219643_at | LRP1B | low density lipoprotein-related protein 1B | 1.792417801 | 0.007571018 |
| | | (deleted in tumors) | | |
| 231991_at | C20orf160 | chromosome 20 open reading frame 160 | 1.790646776 | 0.005210853 |
| 207067_s_at | HDC | histidine decarboxylase | 1.789718357 | 0.001560778 |
| 227397_at | TPM2 | Tropomyosin 2 (beta) | 1.789082546 | 0.003530876 |
| 239294_at | — | Transcribed locus | 1.787442221 | 0.000211428 |
| 209946_at | VEGFC | vascular endothelial growth factor C | 1.785536135 | 0.002132468 |
| 1558517_s_at | LRRC8C | Leucine rich repeat containing 8 family, | 1.778439904 | 0.000634162 |
| | | member C | | |
| 238226_at | FAM70B | Family with sequence similarity 70, | 1.766804458 | 0.003993884 |
| | | member B | | |
| 218542_at | CEP55 | centrosomal protein 55 kDa | 1.766480495 | 0.00456972 |
| 238825_at | ACRC | acidic repeat containing | 1.765644853 | 0.000575832 |
| 226955_at | FLJ36748 | hypothetical protein FLJ36748 | 1.760849448 | 0.007178298 |
| 240729_at | C3orf44 | chromosome 3 open reading frame 44 | 1.759801514 | 0.00907953 |
| 213927_at | MAP3K9 | mitogen-activated protein kinase kinase | 1.758175627 | 0.001073583 |
| | | kinase 9 | | |
| 204677_at | CDH5 | cadherin 5, type 2, VE-cadherin (vascular | 1.756200193 | 0.002844274 |
| | | epithelium) | | |
| 240603_s_at | EXOD1 | exonuclease domain containing 1 | 1.743482609 | 0.006349808 |
| 226488_at | RCCD1 | RCC1 domain containing 1 | 1.74189989 | 0.007700336 |
| 221840_at | PTPRE | protein tyrosine phosphatase, receptor | 1.734758053 | 0.001937708 |
| | | type, E | | |
| 219563_at | C14orf139 | chromosome 14 open reading frame 139 | 1.732968123 | 0.006610821 |
| 206701_x_at | EDNRB | endothelin receptor type B | 1.72696329 | 0.006417885 |
| 230708_at | PRICKLE1 | Prickle-like 1 (Drosophila) | 1.722827481 | 0.00289253 |
| 236262_at | MMRN2 | multimerin 2 | 1.711974151 | 0.003336274 |
| 227307_at | TSPAN18 | Tetraspanin 18 | 1.710373989 | 0.000969376 |
| 228286_at | FLJ40869 | hypothetical protein FLJ40869 | 1.709257729 | 0.000562173 |
| 243502_at | GJA7 | Gap junction protein, alpha 7, 45 kDa | 1.705030419 | 0.008504877 |
| | | (connexin 45) | | |
| 242100_at | CSS3 | chondroitin sulfate synthase 3 | 1.703267729 | 0.007693367 |
| 236213_at | HNRPA3 | Heterogeneous nuclear ribonucleoprotein | 1.701592892 | 0.003804309 |
| | | A3 | | |
| 238070_at | CHD1L | Chromodomain helicase DNA binding | 1.701069331 | 0.002405061 |
| | | protein 1-like | | |
| 229779_at | — | CDNA clone IMAGE: 30367357 | 1.695493306 | 0.002432141 |
| 212012_at | PXDN | peroxidasin homolog (Drosophila) | 1.694006631 | 0.004144948 |
| 204249_s_at | LMO2 | LIM domain only 2 (rhombotin-like 1) | 1.679385873 | 0.000249099 |
| 205507_at | ARHGEF15 | Rho guanine nucleotide exchange factor | 1.669044526 | 0.000690522 |
| | | (GEF) 15 | | |
| 230109_at | PDE7B | phosphodiesterase 7B | 1.668665728 | 0.000140561 |
| 230183_at | — | — | 1.668273388 | 0.005373602 |
| 214831_at | ELK4 | ELK4, ETS-domain protein (SRF accessory | 1.659173491 | 0.002384615 |
| | | protein 1) | | |
| 222344_at | C5orf13 | Chromosome 5 open reading frame 13 | 1.657639092 | 0.000584744 |
| 232715_at | — | CDNA FLJ11544 fis, clone | 1.656166475 | 0.000514145 |
| | | HEMBA1002826 | | |
| 239569_at | FLJ31485 | hypothetical gene supported by AK056047; | 1.65304018 | 0.008755428 |
| | | AK056281; AK123838 | | |
| 225582_at | KIAA1754 | KIAA1754 | 1.652911128 | 0.00374383 |
| 1559021_at | — | — | 1.649156507 | 0.006376851 |
| 232298_at | LOC401093 | hypothetical LOC401093 | 1.643075415 | 0.008841946 |
| 230240_at | DYRK3 | Dual-specificity tyrosine-(Y)- | 1.642306084 | 0.003058381 |
| | | phosphorylation regulated kinase 3 | | |
| 218009_s_at | PRC1 | protein regulator of cytokinesis 1 | 1.640260546 | 0.00301293 |
| 236911_at | FAM80B | Family with sequence similarity 80, | 1.639403991 | 0.009679622 |
| | | member B | | |
| 231047_at | — | Transcribed locus | 1.638091596 | 0.002766661 |
| 208893_s_at | DUSP6 | dual specificity phosphatase 6 | 1.635579771 | 0.006368913 |
| 205150_s_at | KIAA0644 | KIAA0644 gene product | 1.634529995 | 0.001525882 |
| 229172_at | HSPA12B | heat shock 70 kD protein 12B | 1.633459854 | 0.003036325 |
| 211105_s_at | NFATC1 | nuclear factor of activated T-cells, | 1.632076969 | 0.00013047 |
| | | cytoplasmic, calcineurin-dependent 1 | | |
| 204841_s_at | EEA1 | Early endosome antigen 1, 162 kD | 1.631598538 | 0.000634868 |
| 219435_at | C17orf68 | chromosome 17 open reading frame 68 | 1.627241739 | 0.001390091 |
| 228176_at | EDG3 | endothelial differentiation, sphingolipid G- | 1.624734458 | 0.004956598 |
| | | protein-coupled receptor, 3 | | |
| 229241_at | LDHD | lactate dehydrogenase D | 1.624705104 | 0.00169553 |
| 40560_at | TBX2 | T-box 2 | 1.62082772 | 0.001428582 |
| 204844_at | ENPEP | glutamyl aminopeptidase (aminopeptidase | 1.62024314 | 0.006857154 |
| | | A) | | |
| 336_at | TBXA2R | thromboxane A2 receptor | 1.618682048 | 0.003424711 |
| 229802_at | — | CDNA FLJ14388 fis, clone | 1.616940556 | 0.001456126 |
| | | HEMBA1002716 | | |
| 240173_at | — | Transcribed locus | 1.613220042 | 0.002066772 |
| 230384_at | — | — | 1.612422964 | 0.006264468 |
| 206210_s_at | CETP | cholesteryl ester transfer protein, plasma | 1.612157573 | 0.000664016 |
| 229452_at | TMEM88 | transmembrane protein 88 | 1.607813336 | 0.008794922 |
| 234033_at | RAPGEF2 | Rap guanine nucleotide exchange factor | 1.596508119 | 3.12E−05 |
| | | (GEF) 2 | | |
| 242564_at | ZNF587 | Zinc finger protein 587 | 1.59362258 | 0.003272852 |
| 212298_at | NRP1 | neuropilin 1 | 1.585166688 | 0.001414691 |
| 218975_at | COL5A3 | collagen, type V, alpha 3 | 1.583703853 | 0.005780483 |
| 204950_at | CARD8 | caspase recruitment domain family, | 1.580371644 | 0.000169288 |
| | | member 8 | | |
| 206702_at | TEK | TEK tyrosine kinase, endothelial (venous | 1.580244252 | 0.002365161 |
| | | malformations, multiple cutaneous and | | |
| | | mucosal) | | |
| 1558199_at | FN1 | fibronectin 1 | 1.57640983 | 0.000463777 |
| 207869_s_at | CACNA1G | calcium channel, voltage-dependent, alpha | 1.570756454 | 0.009231825 |
| | | 1G subunit | | |
| 223686_at | TPK1 | thiamin pyrophosphokinase 1 | 1.570458879 | 0.000622633 |
| 226984_at | FGD5 | FYVE, RhoGEF and PH domain containing 5 | 1.570065628 | 0.005011733 |
| 202580_x_at | FOXM1 | forkhead box M1 | 1.566237102 | 0.00466876 |
| 219134_at | ELTD1 | EGF, latrophilin and seven transmembrane | 1.565559164 | 0.005439246 |
| | | domain containing 1 | | |
| 213658_at | ZNF710 | Zinc finger protein 710 | 1.561876669 | 0.007465695 |
| 225868_at | TRIM47 | tripartite motif-containing 47 | 1.5551251 | 0.004710451 |
| 243993_at | PCTK2 | PCTAIRE protein kinase 2 | 1.55222323 | 0.00310662 |
| 229581_at | LOC392617 /// LOC641904 | similar to slit homolog 1 /// similar to slit | 1.548358272 | 0.006418746 |
| | | homolog 1 | | |
| 1555973_at | FLJ39051 | Hypothetical gene supported by AK096370 | 1.546294536 | 0.006108847 |
| 229246_at | FLJ44342 | hypothetical protein LOC645460 | 1.545412533 | 0.00325485 |
| 244335_at | — | Transcribed locus | 1.544426744 | 0.007285818 |
| 205111_s_at | PLCE1 | phospholipase C, epsilon 1 | 1.542374231 | 0.004119211 |
| 223595_at | TMEM133 | transmembrane protein 133 | 1.536725529 | 0.002964685 |
| 206638_at | HTR2B | 5-hydroxytryptamine (serotonin) receptor | 1.531058778 | 0.004269334 |
| | | 2B | | |
| 229373_at | — | Transcribed locus | 1.529046215 | 0.008698091 |
| 227165_at | C13orf3 | chromosome 13 open reading frame 3 | 1.527769201 | 0.00097167 |
| 209760_at | KIAA0922 | KIAA0922 | 1.526779028 | 0.001073139 |
| 228308_at | ARF3 | ADP-ribosylation factor 3 | 1.525315553 | 0.005343687 |
| 213519_s_at | LAMA2 | laminin, alpha 2 (merosin, congenital | 1.513408861 | 0.000149444 |
| | | muscular dystrophy) | | |
| 205757_at | ENTPD5 | ectonucleoside triphosphate | 1.512042423 | 0.004064433 |
| | | diphosphohydrolase 5 | | |
| 1552266_at | ADAM32 | ADAM metallopeptidase domain 32 | 1.510690435 | 0.0096982 |
| 226069_at | PRICKLE1 | prickle-like 1 (Drosophila) | 1.508910992 | 0.002013177 |
| 1555520_at | PTCH | patched homolog (Drosophila) | 1.504400498 | 0.007933958 |
| 230593_at | — | — | 1.502837251 | 0.004107145 |
| 226673_at | SH2D3C | SH2 domain containing 3C | 1.500482581 | 0.005066822 |
| 203063_at | PPM1F | protein phosphatase 1F (PP2C domain | 1.498456245 | 0.006272276 |
| | | containing) | | |
| 236330_at | — | CDNA FLJ42688 fis, clone | 1.493434605 | 0.006805918 |
| | | BRAMY3002120 | | |
| 226677_at | ZNF521 | zinc finger protein 521 | 1.491790581 | 0.003529276 |
| 244025_at | — | — | 1.479421042 | 0.006118077 |
| 216840_s_at | LAMA2 | laminin, alpha 2 (merosin, congenital | 1.475490581 | 0.002592941 |
| | | muscular dystrophy) | | |
| 1558820_a_at | C18orf34 | chromosome 18 open reading frame 34 | 1.475140441 | 0.00472897 |
| 209839_at | DNM3 | dynamin 3 | 1.474957866 | 0.008777026 |
| 204845_s_at | ENPEP | glutamyl aminopeptidase (aminopeptidase | 1.470712012 | 0.008560717 |
| | | A) | | |
| 228776_at | GJA7 | gap junction protein, alpha 7, 45 kDa | 1.469835598 | 0.009145995 |
| | | (connexin 45) | | |
| 244825_at | RP11-119E20.1 | KIAA1202 protein | 1.468598501 | 0.000802729 |
| 230266_at | RAB7B | RAB7B, member RAS oncogene family | 1.467706824 | 0.001578978 |
| 235688_s_at | — | Transcribed locus | 1.460149736 | 0.00808911 |
| 230613_at | — | — | 1.459511954 | 0.008686304 |
| 230768_at | FARP2 | FERM, RhoGEF and pleckstrin domain | 1.458687762 | 1.95E−05 |
| | | protein 2 | | |
| 229491_at | LOC133308 | hypothetical protein BC009732 | 1.455421108 | 0.004395867 |
| 236583_at | ABP1 | Amiloride binding protein 1 (amine oxidase | 1.454247358 | 0.004896927 |
| | | (copper-containing)) | | |
| 229893_at | FRMD3 | FERM domain containing 3 | 1.453666333 | 0.00214585 |
| 242582_at | — | Transcribed locus | 1.453282543 | 0.009953388 |
| 37384_at | PPM1F | protein phosphatase 1F (PP2C domain | 1.447825929 | 0.006276245 |
| | | containing) | | |
| 214770_at | MSR1 | macrophage scavenger receptor 1 | 1.447359313 | 8.20E−05 |
| 236034_at | — | — | 1.441276763 | 0.002546565 |
| 209716_at | CSF1 | colony stimulating factor 1 (macrophage) | 1.438610552 | 0.002653507 |
| 1559096_x_at | FBXO9 | F-box protein 9 | 1.437929733 | 0.007314628 |
| 37590_g_at | ZNF710 | Zinc finger protein 710 /// MRNA full length | 1.436178939 | 0.008250509 |
| | | insert cDNA clone EUROIMAGE 375854 | | |
| 226942_at | PHF20L1 | PHD finger protein 20-like 1 | 1.429094948 | 0.002687589 |
| 205116_at | LAMA2 | laminin, alpha 2 (merosin, congenital | 1.426904583 | 0.00319299 |
| | | muscular dystrophy) | | |
| 213739_at | — | CDNA clone IMAGE: 4801297 | 1.426060784 | 0.00696093 |
| 227498_at | — | CDNA FLJ11723 fis, clone | 1.42574381 | 0.002192771 |
| | | HEMBA1005314 | | |
| 1552678_a_at | USP28 | ubiquitin specific peptidase 28 | 1.424245271 | 0.009411156 |
| 243298_at | PSMB5 | Proteasome (prosome, macropain) subunit, | 1.421758878 | 0.009864117 |
| | | beta type, 5 | | |
| 225827_at | EIF2C2 | eukaryotic translation initiation factor 2C, 2 | 1.414679613 | 0.004511353 |
| 235318_at | FBN1 | fibrillin 1 | 1.411403127 | 0.003522421 |
| 229367_s_at | GIMAP6 | GTPase, IMAP family member 6 | 1.409473454 | 0.005453269 |
| 214717_at | DKFZp434H1419 | hypothetical protein DKFZp434H1419 | 1.409064541 | 0.003352216 |
| 239563_at | — | Full-length cDNA clone CL0BB004ZB12 of | 1.408557768 | 0.007665877 |
| | | Neuroblastoma of Homo sapiens (human) | | |
| 225511_at | GPRC5B | G protein-coupled receptor, family C, group | 1.408430305 | 0.004193891 |
| | | 5, member B | | |
| 228618_at | PEAR1 | platelet endothelial aggregation receptor 1 | 1.406131485 | 0.003586462 |
| 243690_at | TRIOBP | TRIO and F-actin binding protein | 1.396104486 | 0.001324248 |
| 219218_at | BAHCC1 | BAH domain and coiled-coil containing 1 | 1.390357901 | 0.001298203 |
| 219253_at | FAM11B | family with sequence similarity 11, member B | 1.386585516 | 0.006402646 |
| 227788_at | — | CDNA FLJ34165 fis, clone FCBBF3014770 | 1.385698744 | 0.00092042 |
| 207554_x_at | TBXA2R | thromboxane A2 receptor | 1.375581733 | 0.00261225 |
| 243482_at | EPS15L1 | Epidermal growth factor receptor pathway | 1.372515471 | 0.005329872 |
| | | substrate 15-like 1 | | |
| 1557576_at | PAQR3 | progestin and adipoQ receptor family | 1.372078865 | 0.006822126 |
| | | member III | | |
| 225618_at | ARHGAP27 | Rho GTPase activating protein 27 | 1.37140617 | 0.00525895 |
| 228983_at | ITGAE | Integrin, alpha E (antigen CD103, human | 1.370165755 | 0.003436716 |
| | | mucosal lymphocyte antigen 1; alpha | | |
| | | polypeptide) | | |
| 239427_at | — | — | 1.370008984 | 0.006044404 |
| 213392_at | MGC35048 | hypothetical protein MGC35048 | 1.366975772 | 0.005727182 |
| 205846_at | PTPRB | protein tyrosine phosphatase, receptor | 1.360043961 | 0.003988738 |
| | | type, B | | |
| 237705_at | — | Transcribed locus | 1.356026297 | 0.002115283 |
| 229698_at | SHANK3 | SH3 and multiple ankyrin repeat domains 3 | 1.349046323 | 0.005162651 |
| 214716_at | BMP2K | BMP2 inducible kinase | 1.337016025 | 0.009656627 |
| 228580_at | HTRA3 | HtrA serine peptidase 3 | 1.336981628 | 0.003070794 |
| 223741_s_at | TTYH2 | tweety homolog 2 (Drosophila) | 1.325392915 | 0.001284367 |
| 213652_at | PCSK5 | Proprotein convertase subtilisin/kexin type 5 | 1.324499982 | 0.007374052 |
| 209410_s_at | GRB10 | growth factor receptor-bound protein 10 | 1.323500709 | 0.00823966 |
| 204944_at | PTPRG | protein tyrosine phosphatase, receptor | 1.322720824 | 0.001896578 |
| | | type, G | | |
| 203144_s_at | KIAA0040 | KIAA0040 | 1.321506495 | 0.008058387 |
| 227032_at | PLXNA2 | plexin A2 | 1.32128317 | 0.002193809 |
| 1564053_a_at | YTHDF3 | YTH domain family, member 3 | 1.320566247 | 0.003413525 |
| 1556583_a_at | — | CDNA FLJ37694 fis, clone BRHIP2015224 | 1.320471098 | 0.006566403 |
| 210105_s_at | FYN | FYN oncogene related to SRC, FGR, YES | 1.320234918 | 0.003299839 |
| 226368_at | CHST11 | Carbohydrate (chondroitin 4) | 1.31805188 | 0.004972789 |
| | | sulfotransferase 11 | | |
| 219874_at | SLC12A8 | solute carrier family 12 (potassium/chloride | 1.316454238 | 0.009067403 |
| | | transporters), member 8 | | |
| 210286_s_at | SLC4A7 | solute carrier family 4, sodium bicarbonate | 1.316066796 | 0.0054996 |
| | | cotransporter, member 7 | | |
| 229824_at | — | CDNA FLJ45325 fis, clone BRHIP3006717 | 1.310641278 | 0.003279718 |
| 236116_at | GPM6B | Glycoprotein M6B | 1.306198936 | 0.004853832 |
| 228976_at | ICOSLG | inducible T-cell co-stimulator ligand | 1.304100307 | 0.005235636 |
| 204337_at | RGS4 | regulator of G-protein signalling 4 | 1.303646814 | 0.006890402 |
| 1565628_at | LRRK1 | Leucine-rich repeat kinase 1 | 1.296491586 | 0.008216571 |
| 211499_s_at | MAPK11 | mitogen-activated protein kinase 11 | 1.294295622 | 0.009542724 |
| 226065_at | PRICKLE1 | prickle-like 1 (Drosophila) | 1.29376582 | 0.004789664 |
| 217585_at | NEBL | nebulette | 1.282408602 | 0.006292427 |
| 207624_s_at | RPGR | retinitis pigmentosa GTPase regulator | 1.27574427 | 0.003805623 |
| 227618_at | FLJ44635 | TPT1-like protein | 1.274572393 | 0.005549593 |
| 230741_at | — | Full length insert cDNA clone YX74D05 | 1.274120834 | 0.005901746 |
| 242677_at | NRP1 | Neuropilin 1 | 1.267676061 | 0.00832135 |
| 227371_at | BAIAP2L1 | BAI1-associated protein 2-like 1 | 1.264718173 | 0.00800131 |
| 232102_at | METTL6 | Methyltransferase like 6 | 1.26113105 | 0.008855962 |
| 1552889_a_at | XTP7 | protein 7 transactivated by hepatitis B virus | 1.258585208 | 0.000383173 |
| | | X antigen (HBxAg) | | |
| 242423_x_at | — | Homo sapiens, clone IMAGE: 4346533, | 1.254498511 | 0.009252861 |
| | | mRNA | | |
| 210287_s_at | FLT1 | fms-related tyrosine kinase 1 (vascular | 1.249552118 | 0.008933646 |
| | | endothelial growth factor/vascular | | |
| | | permeability factor receptor) | | |
| 205841_at | JAK2 | Janus kinase 2 (a protein tyrosine kinase) | 1.2437263 | 0.007601147 |
| 201703_s_at | PPP1R10 | protein phosphatase 1, regulatory subunit | 1.243594711 | 0.008094087 |
| | | 10 | | |
| 1556636_at | — | CDNA FLJ35030 fis, clone | 1.239554971 | 0.008334902 |
| | | OCBBF2015931 | | |
| 224574_at | C17orf49 /// MGC71993 | chromosome 17 open reading frame 49 /// | 1.231159913 | 0.003562667 |
| | | similar to DNA segment, Chr 11, Brigham & | | |
| | | Womens Genetics 0434 expressed | | |
| 1553452_at | MYO1H | myosin IH | 1.227284213 | 0.005690321 |
| 226198_at | TOM1L2 | target of myb1-like 2 (chicken) | 1.226081416 | 0.009555641 |
| 224320_s_at | MCM8 | MCM8 minichromosome maintenance | 1.224592887 | 0.00464148 |
| | | deficient 8 (S. cerevisiae) | | |
| 238854_at | DYNLL1 | Dynein, light chain, LC8-type 1 | 1.222825558 | 0.004973255 |
| 1559524_at | — | MRNA; cDNA DKFZp566N0924 (from | 1.221223651 | 0.009085738 |
| | | clone DKFZp566N0924) | | |
| 242908_x_at | — | Transcribed locus | 1.220981803 | 0.006925595 |
| 218904_s_at | C9orf40 | chromosome 9 open reading frame 40 | 1.220376781 | 0.001274946 |
| 235335_at | ABCA9 | ATP-binding cassette, sub-family A | 1.218834555 | 0.001440673 |
| | | (ABC1), member 9 | | |
| 1555324_at | PTK7 | PTK7 protein tyrosine kinase 7 | 1.214468938 | 0.008815051 |
| 233491_at | LOC285429 | hypothetical protein LOC285429 | 1.212921932 | 0.00693381 |
| 242439_s_at | — | — | 1.210334443 | 0.005618705 |
| 204513_s_at | ELMO1 | engulfment and cell motility 1 | 1.209652265 | 0.004082213 |
| 230276_at | — | — | 1.202292991 | 0.007336101 |
| 205398_s_at | SMAD3 | SMAD, mothers against DPP homolog 3 | 1.19731659 | 0.005711538 |
| | | (Drosophila) | | |
| 236347_at | MMAA | methylmalonic aciduria (cobalamin | 1.197243966 | 0.006949549 |
| | | deficiency) cblA type | | |
| 228837_at | — | CDNA FLJ37747 fis, clone BRHIP2022986 | 1.195748215 | 0.005192664 |
| 209197_at | SYT11 | synaptotagmin XI | 1.195711377 | 0.006304549 |
| 234111_at | — | CDNA: FLJ23063 fis, clone LNG04745 | 1.188617185 | 0.008110156 |
| 232408_at | ZFYVE28 | zinc finger, FYVE domain containing 28 | 1.18766876 | 0.006967438 |
| 240703_s_at | HERC1 | hect (homologous to the E6-AP (UBE3A) | 1.186597557 | 0.004967124 |
| | | carboxyl terminus) domain and RCC1 | | |
| | | (CHC1)-like domain (RLD) 1 | | |
| 209906_at | C3AR1 | complement component 3a receptor 1 | 1.180938532 | 0.000778528 |
| 231932_at | TRAF3IP3 | TRAF3 interacting protein 3 | 1.180781947 | 0.003923476 |
| 201663_s_at | SMC4L1 | SMC4 structural maintenance of | 1.159689804 | 0.009289008 |
| | | chromosomes 4-like 1 (yeast) | | |
| 235398_at | LOC390980 | similar to Zinc finger protein 264 | 1.155731501 | 0.002891267 |
| 205247_at | NOTCH4 | Notch homolog 4 (Drosophila) | 1.148732493 | 0.008057412 |
| 226372_at | CHST11 | Carbohydrate (chondroitin 4) | 1.146606936 | 0.005691488 |
| | | sulfotransferase 11 | | |
| 212521_s_at | PDE8A | phosphodiesterase 8A | 1.13777886 | 0.004007962 |
| 226121_at | MGC23280 | hypothetical protein MGC23280 | 1.134816836 | 0.005433459 |
| 214856_at | SPTBN1 | spectrin, beta, non-erythrocytic 1 | 1.133394795 | 0.004657395 |
| 203002_at | AMOTL2 | angiomotin like 2 | 1.1202371 | 0.007521446 |
| 232009_at | EMR2 | egf-like module containing, mucin-like, | 1.114645546 | 0.004388162 |
| | | hormone receptor-like 2 | | |
| 206481_s_at | LDB2 | LIM domain binding 2 | 1.100829454 | 0.003584449 |
| 233192_s_at | RUFY2 | RUN and FYVE domain containing 2 | 1.100755846 | 0.004976699 |
| 230799_at | LOC150837 | hypothetical protein LOC150837 | 1.098664239 | 0.0071199 |
| 206412_at | FER | fer (fps/fes related) tyrosine kinase | 1.094136456 | 0.003085697 |
| | | (phosphoprotein NCP94) | | |
| 205093_at | PLEKHA6 | pleckstrin homology domain containing, | 1.078926725 | 0.004709964 |
| | | family A member 6 | | |
| 204546_at | KIAA0513 | KIAA0513 | 1.065670932 | 0.006030028 |
| 208072_s_at | DGKD | diacylglycerol kinase, delta 130 kDa | 1.025088133 | 0.005043542 |
| 224923_at | TTC7A | tetratricopeptide repeat domain 7A | 1.018718408 | 0.002881226 |
| 225906_at | — | CDNA FLJ38264 fis, clone FCBBF3001657 | 1.012449179 | 0.006331886 |
| 227882_at | FKRP | fukutin related protein | 1.003236652 | 0.006694083 |
| 1553542_at | KENAE | Kenae | 1.002439167 | 0.008380529 |
| 210160_at | PAFAH1B2 | platelet-activating factor acetylhydrolase, | 0.984243235 | 0.006441233 |
| | | isoform Ib, beta subunit 30 kDa | | |
| 214708_at | SNTB1 | syntrophin, beta 1 (dystrophin-associated | 0.972829307 | 0.008698005 |
| | | protein A1, 59 kDa, basic component 1) | | |
| 241401_at | C4orf12 | chromosome 4 open reading frame 12 | 0.969841391 | 0.006872838 |
| 209409_at | GRB10 | growth factor receptor-bound protein 10 | 0.964792296 | 0.000323924 |
| 236849_at | VTI1A | Vesicle transport through interaction with t- | 0.935973146 | 0.009186297 |
| | | SNAREs homolog 1A (yeast) | | |
| 239498_at | MRRF | Mitochondrial ribosome recycling factor | 0.934608583 | 0.003957275 |
| 238890_at | — | — | 0.929576281 | 0.008919331 |
| 225184_at | ARID1B | AT rich interactive domain 1B (SWI1-like) | 0.928616862 | 0.003787293 |
| 232434_at | DIRC3 | disrupted in renal carcinoma 3 | 0.91828125 | 0.006482536 |
| 222840_at | C14orf103 | Chromosome 14 open reading frame 103 | 0.904046928 | 0.007877339 |
| 209704_at | — | Full length insert cDNA clone YY61D04 | 0.855204308 | 0.005439677 |
| 218615_s_at | TMEM39A | transmembrane protein 39A | 0.780592893 | 0.005384084 |
| 209810_at | SFTPB | surfactant, pulmonary-associated protein B | −0.736517158 | 0.008608591 |
| 1570131_at | LOC285847 | hypothetical protein LOC285847 | −0.813825936 | 0.009140519 |
| 230359_at | KNDC1 | kinase non-catalytic C-lobe domain (KIND) | −0.814718728 | 0.007416943 |
| | | containing 1 | | |
| 1564635_a_at | FHAD1 | forkhead-associated (FHA) | −0.820226404 | 0.007047758 |
| | | phosphopeptide binding domain 1 | | |
| 220414_at | CALML5 | calmodulin-like 5 | −0.823093508 | 0.007377268 |
| 1566906_at | LOC441476 | Hypothetical gene supported by AK097565; | −0.83204424 | 0.007975538 |
| | | BC033939 | | |
| 1553637_s_at | TMCO5 | transmembrane and coiled-coil domains 5 | −0.832230223 | 0.008525668 |
| 208947_s_at | UPF1 | UPF1 regulator of nonsense transcripts | −0.836986163 | 0.008977873 |
| | | homolog (yeast) | | |
| 1553837_at | PGAM5 | phosphoglycerate mutase family member 5 | −0.841159536 | 0.00561017 |
| 238177_at | SLC6A19 | Solute carrier family 6 (neutral amino acid | −0.84457999 | 0.009311556 |
| | | transporter), member 19 | | |
| 232658_at | KIAA1856 | KIAA1856 protein | −0.845101111 | 0.00623322 |
| 240791_at | — | Transcribed locus | −0.85112623 | 0.006267521 |
| 1569293_x_at | MGC12760 | hypothetical protein MGC12760 | −0.85413371 | 0.004952139 |
| 1555675_at | BRCC2 | BRCC2 | −0.858393259 | 0.005880175 |
| 227795_at | NDUFV1 | NADH dehydrogenase (ubiquinone) | −0.858671079 | 0.008014806 |
| | | flavoprotein 1, 51 kDa | | |
| 1553872_at | DKFZp762H185 | hypothetical protein DKFZp762H185 | −0.863313847 | 0.003692242 |
| 211227_s_at | PCDH11Y | protocadherin 11 Y-linked | −0.867605324 | 0.008760076 |
| 240767_x_at | — | — | −0.879528024 | 0.006733675 |
| 1555529_at | RNH1 | ribonuclease/angiogenin inhibitor 1 | −0.879806547 | 0.008113216 |
| 234310_s_at | SUSD2 | sushi domain containing 2 | −0.883047503 | 0.005058793 |
| 1565730_at | — | MRNA; cDNA DKFZp434E145 (from clone | −0.887456225 | 0.004144768 |
| | | DKFZp434E145) | | |
| 1569383_s_at | ZFYVE28 | zinc finger, FYVE domain containing 28 | −0.888309314 | 0.009944314 |
| 215110_at | MBL1P1 | mannose-binding lectin (protein A) 1, | −0.888502378 | 0.004949075 |
| | | pseudogene 1 | | |
| 1561314_at | SCFD2 | Sec1 family domain containing 2 | −0.891110773 | 0.009362009 |
| 206516_at | AMH | anti-Mullerian hormone | −0.894496117 | 0.008322965 |
| 1563224_at | — | Homo sapiens, clone IMAGE: 4516253, | −0.898544354 | 0.00980498 |
| | | mRNA | | |
| 1555053_at | SYT9 | synaptotagmin IX | −0.900261184 | 0.00400231 |
| 211768_at | LAT2 | linker for activation of T cells family, | −0.900265 | 0.009713078 |
| | | member 2 /// linker for activation of T cells | | |
| | | family, member 2 | | |
| 231604_at | — | Transcribed locus | −0.900908256 | 0.005945404 |
| 220438_at | QPCTL | glutaminyl-peptide cyclotransferase-like | −0.903042009 | 0.007042967 |
| 205813_s_at | MAT1A | methionine adenosyltransferase I, alpha | −0.906052421 | 0.006261744 |
| 206083_at | BAI1 | brain-specific angiogenesis inhibitor 1 | −0.906153807 | 0.004622749 |
| 224268_x_at | ZAN | zonadhesin | −0.909622005 | 0.009406064 |
| 1560432_at | UCRP | Usher critical region protein pseudogene | −0.910152444 | 0.006748779 |
| 207896_s_at | DLEC1 | deleted in lung and esophageal cancer 1 | −0.910883746 | 0.009682707 |
| 1555620_a_at | PTGIR | prostaglandin I2 (prostacyclin) receptor (IP) | −0.913128766 | 0.008638353 |
| 229045_at | SLIC1 | selectin ligand interactor cytoplasmic-1 | −0.916108214 | 0.009410318 |
| 203306_s_at | SLC35A1 | solute carrier family 35 (CMP-sialic acid | −0.919488858 | 0.008620732 |
| | | transporter), member A1 | | |
| 211249_at | GPR68 | G protein-coupled receptor 68 | −0.920372315 | 0.005304847 |
| 1569932_at | RP11-262D11.5 | similar to Nance-Horan syndrome protein | −0.925069438 | 0.009967765 |
| 214489_at | FSHB | follicle stimulating hormone, beta | −0.925865652 | 0.00750827 |
| | | polypeptide | | |
| 244343_at | — | CDNA clone IMAGE: 4823238 | −0.926557521 | 0.008084644 |
| 238201_at | — | — | −0.928196576 | 0.005991272 |
| 1557660_s_at | C10orf73 | Chromosome 10 open reading frame 73 | −0.94081106 | 0.006472777 |
| 215583_at | TMEM63A | Transmembrane protein 63A | −0.941571215 | 0.008369239 |
| 216076_at | — | — | −0.944554459 | 0.006004171 |
| 1561420_a_at | — | — | −0.945971366 | 0.009703812 |
| 241481_at | FAM81A | Family with sequence similarity 81, | −0.948483963 | 0.005109485 |
| | | member A | | |
| 209886_s_at | SMAD6 | SMAD, mothers against DPP homolog 6 | −0.949778057 | 0.008630797 |
| | | (Drosophila) | | |
| 205502_at | CYP17A1 | cytochrome P450, family 17, subfamily A, | −0.951992453 | 0.008711385 |
| | | polypeptide 1 | | |
| 214304_x_at | DMN | Desmuslin | −0.952541293 | 0.005877584 |
| 240334_at | — | — | −0.954598189 | 0.005987761 |
| 1568730_at | — | CDNA clone IMAGE: 4822326 | −0.955589487 | 0.006674799 |
| 1565818_s_at | ZNFN1A1 | zinc finger protein, subfamily 1A, 1 (Ikaros) | −0.95646811 | 0.006358412 |
| 1554984_a_at | HLA-DOB | major histocompatibility complex, class II, | −0.958682907 | 0.008756811 |
| | | DO beta | | |
| 239756_at | MAD1L1 | MAD1 mitotic arrest deficient-like 1 (yeast) | −0.961062879 | 0.006807679 |
| 227097_at | — | — | −0.9611128 | 0.004004792 |
| 1564590_a_at | OFCC1 | orofacial cleft 1 candidate 1 | −0.961377744 | 0.003360737 |
| 1566701_at | VRK3 | vaccinia related kinase 3 | −0.961815538 | 0.005307258 |
| 236422_at | FLJ22662 | Hypothetical protein FLJ22662 | −0.961896953 | 0.008581018 |
| 1562694_at | C3orf65 | chromosome 3 open reading frame 65 | −0.962547627 | 0.005560553 |
| 234816_at | C14orf48 | Chromosome 14 open reading frame 48 | −0.977087967 | 0.002065431 |
| 231732_at | SMPD3 | sphingomyelin phosphodiesterase 3, | −0.977713545 | 0.007200417 |
| | | neutral membrane (neutral | | |
| | | sphingomyelinase II) | | |
| 234226_at | OPN4 | opsin 4 (melanopsin) | −0.97832468 | 0.005357738 |
| 242419_at | — | Transcribed locus | −0.983600924 | 0.001674402 |
| 1565556_at | LOC127841 | hypothetical protein LOC127841 | −0.983673653 | 0.008805415 |
| 215574_at | LOC646089 | Hypothetical protein LOC646089 | −0.984156322 | 0.00377295 |
| 238998_x_at | OTUD1 | OTU domain containing 1 | −0.986164557 | 0.003128706 |
| 1558784_at | PARG | Poly (ADP-ribose) glycohydrolase | −0.989336499 | 0.006889531 |
| 236412_at | — | — | −0.993533081 | 0.00830538 |
| 220826_at | C21orf77 | chromosome 21 open reading frame 77 | −0.994142586 | 0.00625721 |
| 1570134_at | FOXP1 | Forkhead box P1 | −0.996246328 | 0.004720583 |
| 240933_at | ETFB | Electron-transfer-flavoprotein, beta | −0.996320655 | 0.008526316 |
| | | polypeptide | | |
| 243960_x_at | GTF2I | General transcription factor II, i | −0.996683181 | 0.002267976 |
| 219109_at | SPAG16 | sperm associated antigen 16 | −0.996910606 | 0.006280093 |
| 1553756_at | C9orf70 | chromosome 9 open reading frame 70 | −0.998682685 | 0.001871501 |
| 223306_at | EBPL | emopamil binding protein-like | −0.999246328 | 0.008467358 |
| 234803_at | CSTL1 | cystatin-like 1 | −1.010488257 | 0.006242025 |
| 215857_at | NCLN | nicalin homolog (zebrafish) | −1.011773765 | 0.004786524 |
| 221138_s_at | — | — | −1.013630151 | 0.004996847 |
| 214523_at | CEBPE | CCAAT/enhancer binding protein (C/EBP), | −1.01386904 | 0.006622733 |
| | | epsilon | | |
| 1554566_at | KCTD17 | potassium channel tetramerisation domain | −1.014742696 | 0.002443985 |
| | | containing 17 | | |
| 225762_x_at | LOC284801 | hypothetical protein LOC284801 | −1.01737355 | 0.002301099 |
| 1559293_x_at | C9orf14 | Chromosome 9 open reading frame 14 | −1.01767793 | 0.008004332 |
| 1552976_at | — | — | −1.01899545 | 0.00976265 |
| 210955_at | CASP10 | caspase 10, apoptosis-related cysteine | −1.021134632 | 0.006642487 |
| | | peptidase | | |
| 1570185_at | — | Homo sapiens, clone IMAGE: 5766850, | −1.02453185 | 0.001801336 |
| | | mRNA | | |
| 234353_at | C19orf15 | chromosome 19 open reading frame 15 | −1.024895504 | 0.003847614 |
| 220849_at | FLJ22659 | hypothetical protein FLJ22659 | −1.025411788 | 0.007671917 |
| 1555410_at | C20orf23 | Chromosome 20 open reading frame 23 | −1.026299801 | 0.005442781 |
| 222185_at | PKNOX2 | PBX/knotted 1 homeobox 2 | −1.027112094 | 0.008483242 |
| 1557588_at | — | CDNA clone IMAGE: 4792258 | −1.027162823 | 0.007855939 |
| 209362_at | SURB7 | SRB7 suppressor of RNA polymerase B | −1.027791307 | 0.005578722 |
| | | homolog (yeast) | | |
| 1560942_at | — | — | −1.030349954 | 0.00214329 |
| 216935_at | C1orf46 | chromosome 1 open reading frame 46 | −1.032153257 | 0.003406291 |
| 223855_s_at | MCHR1 | melanin-concentrating hormone receptor 1 | −1.035700393 | 0.006818558 |
| 1553262_a_at | UTS2R | urotensin 2 receptor | −1.036185132 | 0.003453883 |
| 216372_at | — | — | −1.037784022 | 0.004825906 |
| 203540_at | GFAP | glial fibrillary acidic protein | −1.041951142 | 0.007561087 |
| 236556_s_at | LONRF1 | LON peptidase N-terminal domain and ring | −1.042445876 | 0.00714669 |
| | | finger 1 | | |
| 208173_at | IFNB1 | interferon, beta 1, fibroblast | −1.044967842 | 0.00659603 |
| 1560935_s_at | LOC284669 | hypothetical protein LOC284669 | −1.047944731 | 0.005718901 |
| 244857_at | HBEGF | heparin-binding EGF-like growth factor | −1.049021337 | 0.005870985 |
| 1557755_at | C14orf145 | chromosome 14 open reading frame 145 | −1.049085888 | 0.002020234 |
| 208397_x_at | KCNJ5 | potassium inwardly-rectifying channel, | −1.050161181 | 0.005954393 |
| | | subfamily J, member 5 | | |
| 232499_at | ITPKB | inositol 1,4,5-trisphosphate 3-kinase B | −1.051607294 | 0.008195991 |
| 1566152_a_at | YPEL4 | CDNA FLJ39016 fis, clone NT2RP7001532 | −1.053126202 | 0.005248593 |
| | | /// Yippee-like 4 (Drosophila) | | |
| 220457_at | SAMD4B | sterile alpha motif domain containing 4B | −1.055010859 | 0.004714534 |
| 1554303_at | HNMT | histamine N-methyltransferase | −1.056655291 | 0.007498291 |
| 233956_at | — | Clone 1580375, mRNA sequence | −1.057815528 | 0.008737964 |
| 222381_at | PDCD6 | Programmed cell death 6 | −1.058660948 | 0.002597287 |
| 236554_x_at | TMC8 | transmembrane channel-like 8 | −1.06221888 | 0.003729192 |
| 233998_x_at | — | — | −1.064782568 | 0.00657792 |
| 217214_s_at | SLC6A2 | solute carrier family 6 (neurotransmitter | −1.068639042 | 0.006302745 |
| | | transporter, noradrenalin), member 2 | | |
| 237132_at | TJP2 | tight junction protein 2 (zona occludens 2) | −1.070286323 | 0.004790311 |
| 241514_at | — | — | −1.070928887 | 0.005566597 |
| 210940_s_at | GRM1 | glutamate receptor, metabotropic 1 | −1.074533596 | 0.008337171 |
| 1552337_s_at | HOXD4 | homeobox D4 | −1.076734954 | 0.003153947 |
| 1570052_at | MIPOL1 | Mirror-image polydactyly 1 | −1.078495811 | 0.003017146 |
| 235881_at | FMNL2 | formin-like 2 | −1.079726394 | 0.002172229 |
| 1563005_at | ETNK2 | Ethanolamine kinase 2 | −1.081166394 | 0.008300043 |
| 237523_at | — | — | −1.081416211 | 0.002955301 |
| 1563532_at | HMCN2 | hemicentin 2 | −1.082607593 | 0.003877937 |
| 1557646_at | — | CDNA FLJ34880 fis, clone NT2NE2015885 | −1.083031257 | 0.000931482 |
| 1553062_at | MOGAT1 | monoacylglycerol O-acyltransferase 1 | −1.083706321 | 0.005982594 |
| 243622_at | LOC145694 | hypothetical protein LOC145694 | −1.084021225 | 0.008120703 |
| 223285_s_at | ST6GALNAC4 | ST6 (alpha-N-acetyl-neuraminyl-2,3-beta- | −1.084679624 | 0.007758788 |
| | | galactosyl-1,3)-N-acetylgalactosaminide | | |
| | | alpha-2,6-sialyltransferase 4 | | |
| 205874_at | ITPKA | inositol 1,4,5-trisphosphate 3-kinase A | −1.085540934 | 0.007967413 |
| 206671_at | SAG | S-antigen; retina and pineal gland (arrestin) | −1.08727102 | 0.006713102 |
| 1563822_at | — | CDNA FLJ37602 fis, clone | −1.087487807 | 0.001458726 |
| | | BRCOC2009380 | | |
| 1553880_at | — | — | −1.087894288 | 0.007212023 |
| 240114_s_at | MGC13034 | hypothetical protein MGC13034 | −1.088135297 | 0.009688737 |
| 1564373_a_at | LOC283887 | hypothetical protein LOC283887 | −1.089591363 | 0.00541641 |
| 1558113_at | FAM78B | family with sequence similarity 78, member B | −1.091008742 | 0.002044701 |
| 216847_at | KIAA1660 | KIAA1660 protein | −1.09261481 | 0.003805295 |
| 217660_at | MYH14 | myosin, heavy polypeptide 14 | −1.092910871 | 0.009679526 |
| 1566956_at | OR7E104P | olfactory receptor, family 7, subfamily E, | −1.094839237 | 0.001184239 |
| | | member 104 pseudogene | | |
| 232952_at | DDEF1 | Development and differentiation enhancing | −1.095164757 | 0.002747641 |
| | | factor 1 | | |
| 240102_at | PRSS1 | Protease, serine, 1 (trypsin 1) | −1.095330521 | 0.007762663 |
| 205586_x_at | VGF | VGF nerve growth factor inducible | −1.096131177 | 0.000496646 |
| 234460_at | — | — | −1.097742755 | 0.006879495 |
| 1558844_at | LOC387790 | hypothetical LOC387790 | −1.098773816 | 0.007531716 |
| 233353_at | LOC90342 | similar to fer-1 like protein 3 | −1.102145847 | 0.00810454 |
| 240555_at | MITF | Microphthalmia-associated transcription | −1.102964486 | 0.006916307 |
| | | factor | | |
| 230514_s_at | LYZL1 /// LYZL2 | lysozyme-like 1 /// lysozyme-like 2 | −1.103247298 | 0.001646193 |
| 228866_at | ANKRD11 | Ankyrin repeat domain 11 | −1.103940113 | 0.007723897 |
| 1554954_at | C21orf90 | chromosome 21 open reading frame 90 | −1.104367865 | 0.009852504 |
| 210810_s_at | SLC6A5 | solute carrier family 6 (neurotransmitter | −1.105092796 | 0.003384762 |
| | | transporter, glycine), member 5 | | |
| 216132_at | ASTN2 | Astrotactin 2 | −1.107598698 | 0.00974396 |
| 220337_at | NGB | neuroglobin | −1.107880317 | 0.007902493 |
| 1558679_at | LOC284804 | hypothetical protein LOC284804 | −1.108434045 | 0.008786645 |
| 1558565_at | LOC158267 | hypothetical protein LOC158267 | −1.109680796 | 0.002023811 |
| 233961_at | GABBR2 | Gamma-aminobutyric acid (GABA) B | −1.111198482 | 0.004686369 |
| | | receptor, 2 | | |
| 231593_at | — | Transcribed locus | −1.11145089 | 0.003043426 |
| 232518_at | PRIC285 | peroxisomal proliferator-activated receptor | −1.111522309 | 0.009858333 |
| | | A interacting complex 285 | | |
| 1564315_at | C8orf49 | chromosome 8 open reading frame 49 | −1.112429392 | 0.004051342 |
| 210991_s_at | RIMS3 | regulating synaptic membrane exocytosis 3 | −1.112765744 | 0.004727892 |
| 1553723_at | GPR97 | G protein-coupled receptor 97 | −1.112907722 | 0.00057749 |
| 1561419_at | — | — | −1.113121631 | 0.000830469 |
| 216334_s_at | CYP2A7P1 | cytochrome P450, family 2, subfamily A, | −1.11607699 | 0.006779301 |
| | | polypeptide 7 pseudogene 1 | | |
| 1561266_at | — | CDNA FLJ33873 fis, clone | −1.116142173 | 0.008509106 |
| | | CTONG2006942 | | |
| 234922_s_at | NHSL1 | NHS-like 1 | −1.1176208 | 0.007141439 |
| 220332_at | CLDN16 | claudin 16 | −1.117993379 | 0.006156715 |
| 233104_at | TOMM34 | Translocase of outer mitochondrial | −1.119516558 | 0.009777387 |
| | | membrane 34 | | |
| 240531_at | — | Transcribed locus, weakly similar to | −1.121225694 | 0.004324462 |
| | | XP_516244.1 PREDICTED: similar to | | |
| | | contactin 6; neural adhesion molecule [Pan | | |
| | | troglodytes] | | |
| 236418_at | TTLL10 | tubulin tyrosine ligase-like family, member | −1.122756051 | 0.007206139 |
| | | 10 | | |
| 233962_at | — | — | −1.123058319 | 0.004022995 |
| 220536_at | C14orf115 | chromosome 14 open reading frame 115 | −1.12326735 | 0.007405456 |
| 217247_at | — | — | −1.125054664 | 0.008994854 |
| 1566979_at | KIAA0317 | KIAA0317 | −1.126785705 | 0.001880064 |
| 221670_s_at | LHX3 | LIM homeobox 3 | −1.12755957 | 0.004968541 |
| 1568683_at | MGC23284 | hypothetical protein MGC23284 | −1.129395764 | 0.0079467 |
| 210000_s_at | SOCS1 | suppressor of cytokine signaling 1 | −1.130265802 | 0.002557314 |
| 229379_at | AHDC1 | AT hook, DNA binding motif, containing 1 | −1.131917311 | 0.00859421 |
| 233158_at | KRTHB2 | keratin, hair, basic, 2 | −1.132550548 | 0.002371031 |
| 1559344_at | MAPK10 | Mitogen-activated protein kinase 10 | −1.133654305 | 0.009783084 |
| 1560511_at | — | Homo sapiens, clone IMAGE: 5769051, | −1.134421932 | 0.001350667 |
| | | mRNA | | |
| 233597_at | PNPLA5 | patatin-like phospholipase domain | −1.134898997 | 0.004705281 |
| | | containing 5 | | |
| 217307_at | — | — | −1.135193737 | 0.001603716 |
| 223872_at | EPN1 | Epsin 1 | −1.135354447 | 0.008617476 |
| 238870_at | KCNK9 | CDNA FLJ37082 fis, clone | −1.135537629 | 0.008682624 |
| | | BRACE2016465 /// Potassium channel, | | |
| | | subfamily K, member 9 | | |
| 221181_at | LOC642452 /// LOC651791 | hypothetical protein LOC642452 /// | −1.13738917 | 0.007863467 |
| | | hypothetical protein LOC651791 | | |
| 1552809_at | RFX4 | regulatory factor X, 4 (influences HLA class | −1.140899901 | 0.00460087 |
| | | II expression) | | |
| 1561829_at | SUZ12P | Suppressor of zeste 12 homolog | −1.141283131 | 0.00636054 |
| | | pseudogene | | |
| 219896_at | DRD1IP | dopamine receptor D1 interacting protein | −1.142938589 | 0.008384929 |
| 215914_at | DPP6 | Dipeptidyl-peptidase 6 | −1.148202333 | 0.002021786 |
| 233699_at | EPHB2 | EPH receptor B2 | −1.149741811 | 0.005525386 |
| 1568448_at | RPS6KA3 | Ribosomal protein S6 kinase, 90 kDa, | −1.150133603 | 0.005666618 |
| | | polypeptide 3 | | |
| 244715_at | KIAA0701 | KIAA0701 protein | −1.153880676 | 0.003952519 |
| 220290_at | AIM1L | absent in melanoma 1-like | −1.154353996 | 0.009960904 |
| 1552580_at | FLJ25801 | hypothetical protein FLJ25801 | −1.156753128 | 0.005747423 |
| 239998_at | C10orf53 | chromosome 10 open reading frame 53 | −1.156897208 | 0.003449821 |
| 243793_at | AHDC1 | AT hook, DNA binding motif, containing 1 | −1.160419818 | 0.002881942 |
| 223508_at | NOTCH1 | Notch homolog 1, translocation-associated | −1.161661109 | 0.001940404 |
| | | (Drosophila) | | |
| 207882_at | HSAJ2425 | p65 protein | −1.165249274 | 0.005723664 |
| 206362_x_at | MAP3K10 | mitogen-activated protein kinase kinase | −1.165871362 | 0.003000031 |
| | | kinase 10 | | |
| 208201_at | DUX4 /// DUX3 /// DUX2 /// LOC399839 | double homeobox, 4 /// double homeobox, | −1.165901774 | 0.004739187 |
| | /// LOC440013 /// LOC440014 /// | 3 /// double homeobox, 2 /// similar to | | |
| | LOC440016 /// LOC440017 /// | double homeobox 4c /// similar to double | | |
| | LOC441056 /// DUX4C /// LOC643001 /// | homeobox 4c /// similar to double | | |
| | LOC649385 /// LOC649425 /// | homeobox 4c /// similar to double | | |
| | LOC651299 /// LOC651674 /// | homeobox 4c /// similar to double | | |
| | LOC652119 /// LOC652387 /// | homeobox 4c /// similar to double | | |
| | LOC652505 /// LOC652641 /// | homeobox, 4 /// double homeobox 4c /// | | |
| | LOC653543 /// LOC653544 /// | similar to double homeobox, 4 /// similar to | | |
| | LOC653545 /// LOC653548 /// | double homeobox, 4 /// similar to double | | |
| | LOC653549 | homeobox 4c /// similar to double | | |
| | | homeobox, 4 /// similar to double | | |
| | | homeobox, 4 /// similar to double | | |
| | | homeobox 4c /// similar to double | | |
| | | homeobox 4c /// similar to double | | |
| | | homeobox 4c /// similar to double | | |
| | | homeobox, 4 /// similar to double | | |
| | | homeobox, 4 /// similar to double | | |
| | | homeobox, 4 /// similar to double | | |
| | | homeobox, 4 /// similar to double | | |
| | | homeobox, 4 /// similar to double | | |
| | | homeobox, 4 | | |
| 1559083_x_at | LOC284600 | hypothetical protein LOC284600 | −1.166493494 | 0.008642444 |
| 214423_x_at | — | CDNA FLJ46657 fis, clone | −1.167174106 | 0.00868603 |
| | | TRACH3006264 | | |
| 237078_at | — | Transcribed locus | −1.167954224 | 0.004023882 |
| 210763_x_at | NCR3 | natural cytotoxicity triggering receptor 3 | −1.173662422 | 0.002610161 |
| 231201_at | PTGER1 | Prostaglandin E receptor 1 (subtype EP1), | −1.17367599 | 0.008293065 |
| | | 42 kDa | | |
| 240526_at | ATP11A | ATPase, Class VI, type 11A | −1.174351336 | 0.005005074 |
| 233709_at | KIAA1026 | Kazrin | −1.176229361 | 0.007617105 |
| 237211_x_at | MORN3 | MORN repeat containing 3 | −1.177154086 | 0.005930938 |
| 241054_at | — | Transcribed locus | −1.17754379 | 0.003685534 |
| 1559800_a_at | — | CDNA FLJ36026 fis, clone TESTI2016758 | −1.178833679 | 0.002888089 |
| 237662_at | — | — | −1.180122569 | 0.006014722 |
| 1569449_a_at | CRSP2 | Cofactor required for Sp1 transcriptional | −1.181353623 | 0.000742039 |
| | | activation, subunit 2, 150 kDa | | |
| 230022_at | MGC34761 /// LOC348174 /// | hypothetical protein MGC34761 /// | −1.184833457 | 0.006106163 |
| | LOC497190 | secretory protein LOC348174 /// secretory | | |
| | | protein LOC497190 | | |
| 210487_at | DNTT | deoxynucleotidyltransferase, terminal | −1.185612735 | 0.002932386 |
| 244225_x_at | LMNA | Lamin A/C | −1.188192351 | 0.00181462 |
| 216382_s_at | TNRC21 | trinucleotide repeat containing 21 | −1.188699407 | 0.003332609 |
| 234388_at | TRA@ | T cell receptor alpha locus /// T cell | −1.19227213 | 0.005310958 |
| | | receptor alpha chain /// Rearranged T-cell | | |
| | | receptor alpha-chain mRNA, variable | | |
| | | region | | |
| 1555271_a_at | TERT | telomerase reverse transcriptase | −1.192317431 | 0.004725073 |
| 209987_s_at | ASCL1 | achaete-scute complex-like 1 (Drosophila) | −1.198904511 | 0.001379012 |
| 1555640_at | DAOA | D-amino acid oxidase activator | −1.198957403 | 0.00461604 |
| 220847_x_at | ZNF221 | zinc finger protein 221 | −1.199439588 | 0.001489043 |
| 243674_at | — | — | −1.19998071 | 0.000198592 |
| 234879_at | — | — | −1.201133693 | 0.009803786 |
| 241243_at | RRBP1 | Ribosome binding protein 1 homolog | −1.201714064 | 0.002871141 |
| | | 180 kDa (dog) | | |
| 201374_x_at | PPP2CB | protein phosphatase 2 (formerly 2A), | −1.202211036 | 0.009665476 |
| | | catalytic subunit, beta isoform | | |
| 1567682_x_at | SNORA74A | small nucleolar RNA, H/ACA box 74A | −1.202908814 | 0.002953621 |
| 237242_at | LOC126536 | hypothetical protein LOC126536 | −1.203459331 | 0.005764377 |
| 216937_s_at | RS1 | retinoschisis (X-linked, juvenile) 1 | −1.203611638 | 0.007985697 |
| 222247_at | DXS542 | putative X-linked retinopathy protein | −1.206486752 | 0.008719723 |
| 215328_at | KIAA0953 | KIAA0953 | −1.207715719 | 0.004148102 |
| 239610_at | ASB13 | Ankyrin repeat and SOCS box-containing | −1.208669352 | 0.003662086 |
| | | 13 | | |
| 1553927_at | C7orf33 | chromosome 7 open reading frame 33 | −1.20975134 | 0.003766232 |
| 216688_at | SH3TC1 | SH3 domain and tetratricopeptide repeats 1 | −1.209981325 | 0.009709123 |
| 237559_at | GPR55 | G protein-coupled receptor 55 | −1.211332184 | 0.007281599 |
| 217413_s_at | TNXB | tenascin XB | −1.212930978 | 0.005842923 |
| 221401_at | CACNG5 | calcium channel, voltage-dependent, | −1.213355599 | 0.005316983 |
| | | gamma subunit 5 | | |
| 1565935_at | LOC91431 | prematurely terminated mRNA decay | −1.214508086 | 0.000401806 |
| | | factor-like | | |
| 237117_at | — | Full length insert cDNA clone ZE03A08 | −1.216077851 | 0.00339918 |
| 217000_at | — | — | −1.216254203 | 0.008416605 |
| 241270_at | RHBDF2 | Rhomboid 5 homolog 2 (Drosophila) | −1.218326467 | 0.00171809 |
| 233143_at | C20orf173 | chromosome 20 open reading frame 173 | −1.218857095 | 0.003018158 |
| 242987_x_at | — | — | −1.220327272 | 0.006399007 |
| 1569750_at | SNRPN | Small nuclear ribonucleoprotein | −1.220496782 | 0.00159057 |
| | | polypeptide N | | |
| 234361_at | CREB3L3 | cAMP responsive element binding protein | −1.222027919 | 0.009436502 |
| | | 3-like 3 | | |
| 1554961_at | FGFR4 | fibroblast growth factor receptor 4 | −1.223270262 | 0.001352491 |
| 1555792_a_at | FLJ36046 | hypothetical protein FLJ36046 | −1.223402512 | 0.008383515 |
| 237042_at | — | Transcribed locus | −1.228362889 | 0.005847271 |
| 216461_at | C9orf106 | Chromosome 9 open reading frame 106 | −1.228869461 | 0.003320141 |
| 1566471_at | — | — | −1.230371576 | 0.002138838 |
| 224328_s_at | LCE3D | late cornified envelope 3D /// late cornified | −1.233515532 | 0.008223024 |
| | | envelope 3D | | |
| 213439_x_at | RPIP8 | RaP2 interacting protein 8 | −1.233540669 | 0.003103182 |
| 211407_at | NDUFB7 | NADH dehydrogenase (ubiquinone) 1 beta | −1.233626444 | 0.003193364 |
| | | subcomplex, 7, 18 kDa | | |
| 207083_s_at | CC2D1A | coiled-coil and C2 domain containing 1A | −1.233923772 | 0.006889943 |
| 221105_at | — | — | −1.235492084 | 0.00219785 |
| 1569780_at | — | CDNA clone IMAGE: 4824322 | −1.23724242 | 0.006515852 |
| 240507_at | — | CDNA FLJ46224 fis, clone TESTI4014262 | −1.237485612 | 0.001256507 |
| 220377_at | FAM30A | family with sequence similarity 30, member A | −1.240236065 | 0.009167891 |
| 234517_at | — | — | −1.240392219 | 0.006628871 |
| 1553540_a_at | SLC29A2 | solute carrier family 29 (nucleoside | −1.241379364 | 0.003605764 |
| | | transporters), member 2 | | |
| 235994_s_at | SNRPE | Small nuclear ribonucleoprotein | −1.241453059 | 0.000404673 |
| | | polypeptide E | | |
| 221254_s_at | PITPNM3 | PITPNM family member 3 /// PITPNM | −1.242623022 | 0.000331944 |
| | | family member 3 | | |
| 1553859_at | TPH1 | tryptophan hydroxylase 1 (tryptophan 5- | −1.245337182 | 0.007002443 |
| | | monooxygenase) | | |
| 237804_at | DNAH11 | Dynein, axonemal, heavy polypeptide 11 | −1.246031685 | 0.009676391 |
| 232739_at | — | CDNA: FLJ21766 fis, clone COLF7179 | −1.247015249 | 0.009340928 |
| 1565908_at | NADSYN1 | NAD synthetase 1 | −1.248230106 | 0.007161591 |
| 1556502_at | RP11-151A6.2 | Hypothetical protein BC004360 | −1.249578942 | 0.00228734 |
| 243854_at | APLP2 | Amyloid beta (A4) precursor-like protein 2 | −1.250299604 | 0.000110018 |
| 1562002_at | MYOZ3 | myozenin 3 | −1.250974118 | 0.007517124 |
| 1563946_at | — | — | −1.251503961 | 0.006030744 |
| 220109_at | TF | transferrin | −1.25192163 | 0.009832468 |
| 208332_at | PRY | PTPN13-like, Y-linked | −1.252733061 | 0.001540441 |
| 214524_at | GHRH | growth hormone releasing hormone | −1.253938535 | 0.003766087 |
| 232722_at | RNASET2 | ribonuclease T2 | −1.255245836 | 0.002921784 |
| 1554501_at | TSC22D4 | TSC22 domain family, member 4 | −1.25549119 | 0.004291601 |
| 1556145_a_at | BAIAP2 | BAI1-associated protein 2 | −1.25724077 | 0.003743781 |
| 216493_s_at | IGF2BP3 /// LOC651107 | insulin-like growth factor 2 mRNA binding | −1.258896728 | 0.005033312 |
| | | protein 3 /// similar to IGF-II mRNA-binding | | |
| | | protein 3 | | |
| 233747_at | — | — | −1.260268761 | 0.008887364 |
| 242367_at | — | — | −1.261165136 | 0.001043383 |
| 207139_at | ATP4A | ATPase, H+/K+ exchanging, alpha | −1.263045405 | 0.002187127 |
| | | polypeptide | | |
| 231189_at | — | — | −1.263320949 | 0.000509582 |
| 240534_at | — | — | −1.2652905 | 0.007439387 |
| 203808_at | — | — | −1.266679311 | 0.000685717 |
| 216409_at | ACSL6 | Acyl-CoA synthetase long-chain family | −1.266913737 | 0.009111438 |
| | | member 6 | | |
| 203491_s_at | CEP57 | centrosomal protein 57 kDa | −1.267623742 | 0.005776999 |
| 215170_s_at | CEP152 | centrosomal protein 152 kDa | −1.268632761 | 0.003838739 |
| 206280_at | CDH18 | cadherin 18, type 2 | −1.270723977 | 0.006433501 |
| 1560001_at | LOC389634 | Hypothetical LOC389634 | −1.273690106 | 0.004200703 |
| 1554476_x_at | LOC388558 | hypothetical gene supported by BC033230 | −1.27399118 | 0.005149469 |
| 210242_x_at | MTHFS | 5,10-methenyltetrahydrofolate synthetase | −1.276194549 | 0.00105063 |
| | | (5-formyltetrahydrofolate cyclo-ligase) | | |
| 1554065_at | C9orf28 | chromosome 9 open reading frame 28 | −1.27818958 | 0.004605322 |
| 215871_at | PLA2G5 | phospholipase A2, group V | −1.279279916 | 0.007421001 |
| 240630_at | RARA | Retinoic acid receptor, alpha | −1.282703336 | 0.001953079 |
| 217277_at | — | — | −1.284137051 | 0.004976225 |
| 231602_at | PRRX2 | Paired related homeobox 2 | −1.284148559 | 0.005877254 |
| 1561277_at | LOC339298 | hypothetical protein LOC339298 | −1.284723247 | 0.003150237 |
| 1568871_at | — | — | −1.285638647 | 0.002828604 |
| 208558_at | OR10H1 | olfactory receptor, family 10, subfamily H, | −1.287052583 | 0.007635844 |
| | | member 1 | | |
| 208085_s_at | ARHGAP6 | Rho GTPase activating protein 6 | −1.289363306 | 0.002117655 |
| 243635_at | — | Transcribed locus | −1.290280624 | 0.009234129 |
| 221414_s_at | DEFB126 | defensin, beta 126 /// defensin, beta 126 | −1.290577676 | 0.00543412 |
| 1554214_at | LOC644719 /// LOC651522 | hypothetical protein LOC644719 /// | −1.291051398 | 0.009508982 |
| | | hypothetical protein LOC651522 | | |
| 215664_s_at | EPHA5 | EPH receptor A5 | −1.293961114 | 0.006974394 |
| 237595_at | LMX1A | LIM homeobox transcription factor 1, alpha | −1.294100259 | 0.006392616 |
| 1553093_a_at | DEFB119 | defensin, beta 119 | −1.294632542 | 0.001848034 |
| 1555016_at | IL16 | interleukin 16 (lymphocyte chemoattractant | −1.294739332 | 0.000946037 |
| | | factor) | | |
| 1552724_at | OTEX | paired-like homeobox protein OTEX | −1.295504668 | 0.005935657 |
| 230829_at | CST9L | cystatin 9-like (mouse) | −1.295715909 | 0.002717942 |
| 1568746_a_at | LOC646268 | hypothetical protein LOC646268 | −1.297108099 | 0.005418864 |
| 1563452_at | KIAA0241 | KIAA0241 | −1.297556087 | 0.005637825 |
| 1566690_at | — | MRNA; cDNA DKFZp667C031 (from clone | −1.299999407 | 0.006497658 |
| | | DKFZp667C031) | | |
| 1562827_at | — | Homo sapiens, clone IMAGE: 5167652, | −1.30144825 | 0.002690935 |
| | | mRNA | | |
| 1559261_a_at | PKD1L2 | polycystic kidney disease 1-like 2 | −1.305039812 | 0.000351806 |
| 241512_at | SPATC1 | spermatogenesis and centriole associated 1 | −1.30525767 | 0.009232083 |
| 216974_at | KITLG | KIT ligand | −1.30543832 | 0.001097682 |
| 1566456_at | — | MRNA; cDNA DKFZp667F034 (from clone | −1.305965894 | 0.004507342 |
| | | DKFZp667F034) | | |
| 1569954_at | C10orf112 | Chromosome 10 open reading frame 112 | −1.306027456 | 0.009215467 |
| 240514_at | LOC643847 | Similar to Pepsin A precursor | −1.307238388 | 0.002581966 |
| 237959_at | HUNK | Hormonally upregulated Neu-associated | −1.307931642 | 0.009490426 |
| | | kinase | | |
| 220630_s_at | CHIA | chitinase, acidic | −1.308089382 | 0.007362181 |
| 238354_x_at | DIP2C | DIP2 disco-interacting protein 2 homolog C | −1.309166702 | 0.00098194 |
| | | (Drosophila) | | |
| 211400_at | FGFR2 | fibroblast growth factor receptor 2 | −1.309267445 | 0.005915574 |
| | | (bacteria-expressed kinase, keratinocyte | | |
| | | growth factor receptor, craniofacial | | |
| 242159_at | — | dysostosis 1, Crouzon syndrome, Pfeiffer | −1.30973061 | 0.009114157 |
| | | syndrome, Jackson-Weiss syndrome) | | |
| | | Transcribed locus, weakly similar to | | |
| | | XP_516250.1 PREDICTED: hypothetical | | |
| | | protein XP_516250 [Pan troglodytes] | | |
| 1555304_a_at | KCNH5 | potassium voltage-gated channel, | −1.310641664 | 0.003713684 |
| | | subfamily H (eag-related), member 5 | | |
| 233030_at | PNPLA3 | patatin-like phospholipase domain | −1.312039028 | 0.006702313 |
| | | containing 3 | | |
| 1560300_a_at | LOC653349 | similar to DMRT-like family C1 | −1.312241334 | 0.00950098 |
| 1569990_at | NUDT3 | nudix (nucleoside diphosphate linked | −1.312675069 | 0.008913594 |
| | | moiety X)-type motif 3 | | |
| 236151_at | KIAA1853 | KIAA1853 | −1.317265712 | 0.003972033 |
| 235586_at | GNA12 | Guanine nucleotide binding protein (G | −1.320291256 | 0.005315245 |
| | | protein) alpha 12 | | |
| 216679_at | — | — | −1.321570705 | 0.002951749 |
| 1563121_at | — | MRNA; cDNA DKFZp586B1221 (from | −1.322015196 | 0.005067129 |
| | | clone DKFZp586B1221) | | |
| 1554974_at | ACY3 | aspartoacylase (aminocyclase) 3 | −1.323921124 | 0.003621005 |
| 210745_at | ONECUT1 | one cut domain, family member 1 | −1.324715321 | 0.009743515 |
| 223509_at | CLDN2 | claudin 2 | −1.326377104 | 0.007600072 |
| 241517_at | DDEF1 | Development and differentiation enhancing | −1.327283918 | 0.004587708 |
| | | factor 1 | | |
| 216579_at | GJB4 | gap junction protein, beta 4 (connexin 30.3) | −1.329784938 | 0.004571873 |
| 243518_at | — | — | −1.330915977 | 0.005245078 |
| 208584_at | SNCG | synuclein, gamma (breast cancer-specific | −1.332661289 | 0.004678377 |
| | | protein 1) | | |
| 214479_at | GFRA3 | GDNF family receptor alpha 3 | −1.332705464 | 0.00270263 |
| 1567540_at | SPAG10 | sperm associated antigen 10 | −1.333092116 | 0.005840848 |
| 1559640_at | ANKFN1 | Ankyrin-repeat and fibronectin type III | −1.333161707 | 0.009890763 |
| | | domain containing 1 | | |
| 215449_at | BZRPL1 | benzodiazapine receptor (peripheral)-like 1 | −1.333428745 | 0.005197324 |
| 1564407_a_at | DOCK1 | Dedicator of cytokinesis 1 | −1.336706535 | 0.001878062 |
| 243870_at | — | Transcribed locus | −1.339209988 | 0.002842249 |
| 222865_x_at | C16orf30 | chromosome 16 open reading frame 30 | −1.342143112 | 0.009523841 |
| 1562604_at | — | CDNA FLJ37024 fis, clone | −1.342213743 | 0.008326321 |
| | | BRACE2010837 | | |
| 1564129_a_at | QPRT | Quinolinate phosphoribosyltransferase | −1.345237572 | 0.009151454 |
| | | (nicotinate-nucleotide pyrophosphorylase | | |
| | | (carboxylating)) | | |
| 217017_at | OSBPL10 | oxysterol binding protein-like 10 | −1.349310054 | 0.002751959 |
| 1558937_s_at | SMA3 | SMA3 | −1.35420635 | 0.002042878 |
| 1566957_at | OR7E104P | olfactory receptor, family 7, subfamily E, | −1.355118935 | 0.000154704 |
| | | member 104 pseudogene | | |
| 242510_at | DHDDS | Dehydrodolichyl diphosphate synthase | −1.357285305 | 0.003124919 |
| 232869_at | SRGAP3 | SLIT-ROBO Rho GTPase activating protein 3 | −1.360843786 | 0.00256807 |
| 233920_at | MAD1L1 | MAD1 mitotic arrest deficient-like 1 (yeast) | −1.361177989 | 0.000810032 |
| 1566670_at | C21orf124 | chromosome 21 open reading frame 124 | −1.363189109 | 0.004533006 |
| 241104_at | SORBS2 | Sorbin and SH3 domain containing 2 | −1.363372413 | 0.007434433 |
| 239907_at | KIAA1026 | Kazrin | −1.363614055 | 0.007470933 |
| 206386_at | SERPINA7 | serpin peptidase inhibitor, clade A (alpha-1 | −1.364605584 | 0.009329656 |
| | | antiproteinase, antitrypsin), member 7 | | |
| 1560767_at | HCG22 | HLA complex group 22 | −1.366348884 | 0.004931574 |
| 1565874_at | KIAA1279 | KIAA1279 | −1.367739446 | 0.003583513 |
| 237659_at | FLJ40288 | hypothetical protein FLJ40288 | −1.368425519 | 0.000922438 |
| 208524_at | GPR15 | G protein-coupled receptor 15 | −1.369272806 | 0.004327285 |
| 217313_at | — | — | −1.370510299 | 0.001357014 |
| 1557404_at | — | CDNA clone IMAGE: 5266464 | −1.375812486 | 0.004966838 |
| 243097_x_at | — | Transcribed locus | −1.378521082 | 0.003147545 |
| 231089_at | — | CDNA FLJ34446 fis, clone | −1.381060122 | 0.001519995 |
| | | HLUNG2002050 | | |
| 1553399_a_at | C17orf69 | chromosome 17 open reading frame 69 | −1.381299757 | 0.008124172 |
| 206162_x_at | SYT5 | synaptotagmin V | −1.383832023 | 0.00398533 |
| 203539_s_at | — | — | −1.384349224 | 0.001950442 |
| 1558868_a_at | SART2 | Squamous cell carcinoma antigen | −1.384553293 | 9.59E−05 |
| | | recognized by T cells 2 | | |
| 1554937_x_at | LOC283849 | hypothetical protein LOC283849 | −1.384630509 | 0.004916567 |
| 244475_at | LOC646951 | Similar to CG14142-PA | −1.386940792 | 0.008308707 |
| 1560576_at | — | CDNA clone IMAGE: 4303615 | −1.389860744 | 0.009541161 |
| 236877_at | DOCK1 | Dedicator of cytokinesis 1 | −1.393786723 | 0.006909506 |
| 239870_at | SPATS1 | spermatogenesis associated, serine-rich 1 | −1.39542902 | 0.002674062 |
| 1552761_at | SLC16A11 | solute carrier family 16 (monocarboxylic | −1.404214273 | 0.003829528 |
| | | acid transporters), member 11 | | |
| 234820_at | MAS1L | MAS1 oncogene-like | −1.405743855 | 0.004764705 |
| 1561770_at | RPL7L1 | Ribosomal protein L7-like 1 | −1.408647037 | 0.001509518 |
| 211531_x_at | PRB1 /// PRB2 | proline-rich protein BstNI subfamily 1 /// | −1.412120376 | 0.001480363 |
| | | proline-rich protein BstNI subfamily 2 | | |
| 215325_x_at | C19orf26 | chromosome 19 open reading frame 26 | −1.416096791 | 0.00722469 |
| 1570040_at | — | CDNA clone IMAGE: 5267944 | −1.416923143 | 0.006452554 |
| 1557790_at | JRKL | Jerky homolog-like (mouse) | −1.419080063 | 0.006754774 |
| 210789_x_at | CEACAM3 | carcinoembryonic antigen-related cell | −1.420013147 | 0.000782642 |
| | | adhesion molecule 3 | | |
| 208495_at | TLX3 | T-cell leukemia homeobox 3 | −1.423650533 | 0.002102486 |
| 1553846_at | SPERT | spermatid associated | −1.423985598 | 0.000564489 |
| 236199_at | ALOX5 | Arachidonate 5-lipoxygenase | −1.424631265 | 0.000844732 |
| 240545_at | LOC286382 | hypothetical protein LOC286382 | −1.424635379 | 0.003904398 |
| 216419_at | CROCC | ciliary rootlet coiled-coil, rootletin | −1.42473085 | 0.002672176 |
| 1569276_at | VAC14 | Vac14 homolog (S. cerevisiae) | −1.429917321 | 0.003286548 |
| 216512_s_at | DCT | dopachrome tautomerase (dopachrome | −1.432285677 | 0.000746133 |
| | | delta-isomerase, tyrosine-related protein 2) | | |
| 239993_at | LOC390705 /// LOC647152 /// | similar to protein phosphatase 2A 48 kDa | −1.435330608 | 0.006460411 |
| | LOC647205 /// LOC647208 | regulatory subunit isoform 1; | | |
| | | serine/threonine protein phosphatase 2A, | | |
| | | 48 kDa regulatory subunit; PP2A, subunit B, | | |
| | | PR48 isoform; PP2A B subunit PR48; NY- | | |
| | | REN-8 antigen /// similar to protein | | |
| | | phosphatase 2, regulatory subunit B, beta | | |
| | | isoform 1 /// similar to protein phosphatase | | |
| | | 2, regulatory subunit B, beta isoform 1 /// | | |
| | | similar to similar to protein phosphatase 2A | | |
| | | 48 kDa regulatory subunit isoform 1; | | |
| | | serine/threonine protein phosphatase 2A, | | |
| | | 48 kDa regulatory subunit; PP2A, subunit B, | | |
| | | PR48 isoform; PP2A B subunit PR48; NY- | | |
| | | REN-8 antigen | | |
| 208341_x_at | CSH2 | chorionic somatomammotropin hormone 2 | −1.436770215 | 0.005110357 |
| 1564823_at | CDC42BPG | CDC42 binding protein kinase gamma | −1.438094857 | 0.001604573 |
| | | (DMPK-like) | | |
| 216244_at | IL1RN | interleukin 1 receptor antagonist | −1.438207981 | 0.003347333 |
| 1553213_a_at | K5B | keratin 5b | −1.441395639 | 0.000653515 |
| 210584_s_at | CTA-126B4.3 /// POLDIP3 /// dJ222E13.2 | CGI-96 protein /// polymerase (DNA- | −1.445411508 | 7.24E−05 |
| | | directed), delta interacting protein 3 /// | | |
| | | similar to CGI-96 | | |
| 240826_at | — | Transcribed locus | −1.44592078 | 0.000628221 |
| 1555985_at | C17orf64 | chromosome 17 open reading frame 64 | −1.446895225 | 0.005900732 |
| 1564985_a_at | SLC8A1 | solute carrier family 8 (sodium/calcium | −1.451028332 | 0.005257547 |
| | | exchanger), member 1 | | |
| 220714_at | PRDM14 | PR domain containing 14 | −1.451977495 | 0.006341637 |
| 1570366_x_at | ZNF709 | zinc finger protein 709 | −1.453296453 | 0.006942014 |
| 1555465_at | MCOLN2 | mucolipin 2 | −1.454338929 | 0.005390639 |
| 216690_at | OR7C1 | olfactory receptor, family 7, subfamily C, | −1.459052708 | 1.05E−05 |
| | | member 1 | | |
| 207533_at | CCL1 | chemokine (C-C motif) ligand 1 | −1.460019904 | 0.001874018 |
| 1555682_at | OTUB2 | OTU domain, ubiquitin aldehyde binding 2 | −1.461101258 | 0.000276073 |
| 1561165_a_at | DEFB108B /// LOC644407 /// | defensin, beta 108B /// similar to Beta- | −1.461489312 | 0.007341093 |
| | LOC645413 /// LOC645826 /// | defensin 108B precursor (Defensin, beta | | |
| | LOC652133 | 108B) (Defensin, beta 108) (Beta-defensin | | |
| | | 8) (DEFB-8) (BD-8) (hBD-8) /// similar to | | |
| | | Beta-defensin 108B precursor (Defensin, | | |
| | | beta 108B) (Defensin, beta 108) (Beta- | | |
| | | defensin 8) (DEFB-8) (BD-8) (hBD-8) /// | | |
| | | similar to Beta-defensin 108B precursor | | |
| | | (Defensin, beta 108B) (Defensin, beta 108) | | |
| | | (Beta-defensin 8) (DEFB-8) (BD-8) (hBD-8) | | |
| | | /// similar to Beta-defensin 108B precursor | | |
| | | (Defensin, beta 108B) (Defensin, beta 108) | | |
| | | (Beta-defensin 8) (DEFB-8) (BD-8) (hBD-8) | | |
| 1438_at | EPHB3 | EPH receptor B3 | −1.461865046 | 0.002447151 |
| 1563494_at | NRG3 | Neuregulin 3 | −1.462703083 | 0.000745943 |
| 237851_at | NIBP | NIK and IKK{beta} binding protein | −1.46478559 | 0.002694882 |
| 208588_at | FKSG2 | apoptosis inhibitor | −1.466144405 | 0.000158951 |
| 244796_at | — | — | −1.466896321 | 0.003331279 |
| 232475_at | C15orf42 | chromosome 15 open reading frame 42 | −1.468132202 | 0.004845495 |
| 1570537_a_at | — | Homo sapiens, clone IMAGE: 4429647, | −1.47001941 | 0.007556303 |
| | | mRNA | | |
| 1562630_at | FLJ37543 | Hypothetical protein FLJ37543 | −1.470495805 | 0.001033607 |
| 233891_at | — | — | −1.477560318 | 0.005973465 |
| 1557649_at | GALE | UDP-galactose-4-epimerase | −1.478797748 | 0.00099248 |
| 239583_x_at | PLAC7 | placenta-specific 7 | −1.47955112 | 0.000854518 |
| 240364_at | — | Transcribed locus, weakly similar to | −1.482502766 | 0.006885333 |
| | | XP_509552.1 PREDICTED: similar to | | |
| | | TPTE and PTEN homologous inositol lipid | | |
| | | phosphatase isoform gamma; TPTE and | | |
| | | PTEN homologous inositol lipid | | |
| | | phosphatase [Pan troglodytes] | | |
| 211918_x_at | PAPPA2 | pappalysin 2 /// pappalysin 2 | −1.484511511 | 0.009987368 |
| 243758_at | CCDC37 | coiled-coil domain containing 37 | −1.489759 | 0.006969616 |
| 240944_at | — | CDNA clone IMAGE: 5532261 | −1.4907953 | 0.001189996 |
| 240431_at | — | — | −1.491242872 | 0.009341778 |
| 243620_at | RP11-151A6.2 | Hypothetical protein BC004360 | −1.499202798 | 0.000368171 |
| 1562942_at | — | Homo sapiens, clone IMAGE: 5180681, | −1.501146059 | 0.002830481 |
| | | mRNA | | |
| 1560866_at | WNK2 | WNK lysine deficient protein kinase 2 | −1.503113158 | 0.0020748 |
| 1553264_a_at | SYN1 | synapsin I | −1.50456326 | 0.002076693 |
| 1552430_at | WDR17 | WD repeat domain 17 | −1.507986039 | 0.007787854 |
| 216040_x_at | RAB11FIP3 | RAB11 family interacting protein 3 (class II) | −1.50851208 | 0.00691463 |
| 1552459_a_at | MBD3L1 | methyl-CpG binding domain protein 3-like 1 | −1.510179057 | 0.00310721 |
| 1557465_at | RP11-327P2.4 | hypothetical protein FLJ37307 | −1.513363981 | 0.001287496 |
| 1560384_a_at | SCFD2 | Sec1 family domain containing 2 | −1.513819471 | 0.008681425 |
| 242872_at | CIT | citron (rho-interacting, serine/threonine | −1.522887679 | 0.000330863 |
| | | kinase 21) | | |
| 1553288_a_at | FLJ37538 | hypothetical protein FLJ37538 | −1.524336425 | 0.005852833 |
| 213990_s_at | PAK7 | p21(CDKN1A)-activated kinase 7 | −1.532647282 | 0.002349757 |
| 1562004_x_at | MYOZ3 | myozenin 3 | −1.53519541 | 0.001041124 |
| 1566043_at | INCENP | Inner centromere protein antigens | −1.538539555 | 0.006090911 |
| | | 135/155 kDa | | |
| 1569274_at | LOC283551 | Hypothetical protein LOC283551 | −1.539594295 | 0.008323691 |
| 234473_at | RP11-408E5.4 /// LOC650459 | similar to CDC42-binding protein kinase | −1.54557293 | 0.004796404 |
| | | beta /// similar to CDC42-binding protein | | |
| | | kinase beta | | |
| 1563728_at | C9orf14 | chromosome 9 open reading frame 14 | −1.550906232 | 0.000949991 |
| 1560806_at | LOC150527 | hypothetical protein LOC150527 | −1.558841845 | 0.009023365 |
| 1554618_at | HRBL | HIV-1 Rev binding protein-like | −1.56195361 | 0.000524868 |
| 205531_s_at | GLS2 | glutaminase 2 (liver, mitochondrial) | −1.573589268 | 0.003642197 |
| 1559252_a_at | C20orf29 | Chromosome 20 open reading frame 29 | −1.574180251 | 0.0011658 |
| 217182_at | MUC5AC | mucin 5AC, oligomeric mucus/gel-forming | −1.577415833 | 0.005411604 |
| 232626_at | TAS2R14 | Taste receptor, type 2, member 14 | −1.57747242 | 0.006342715 |
| 214536_at | SLURP1 | secreted LY6/PLAUR domain containing 1 | −1.579736484 | 0.004045498 |
| 240614_at | KCNC2 | potassium voltage-gated channel, Shaw- | −1.585955913 | 0.007096179 |
| | | related subfamily, member 2 | | |
| 1556477_a_at | LOC283485 | hypothetical protein LOC283485 | −1.588360727 | 0.000959703 |
| 1555748_x_at | CD79B | CD79b molecule, immunoglobulin- | −1.593916248 | 0.004037095 |
| | | associated beta | | |
| 216063_at | HBBP1 | hemoglobin, beta pseudogene 1 /// | −1.594778521 | 0.002810799 |
| | | hemoglobin, beta pseudogene 1 | | |
| 1561318_at | — | CDNA clone IMAGE: 5287025 | −1.611047838 | 0.004849671 |
| 239706_x_at | FLJ30307 | Hypothetical gene supported by AK054869 | −1.625903857 | 0.001997412 |
| 1567270_at | — | — | −1.626406391 | 0.005296223 |
| 1553812_at | TLE6 | transducin-like enhancer of split 6 (E(sp1) | −1.627524712 | 0.002059748 |
| | | homolog, Drosophila) | | |
| 1563045_at | — | CDNA clone IMAGE: 4827232 | −1.628765895 | 0.006316254 |
| 1570127_at | — | CDNA clone IMAGE: 5273406 | −1.652199643 | 0.003146749 |
| 1562919_at | FAM45A | Family with sequence similarity 45, | −1.653907952 | 0.007895854 |
| | | member A | | |
| 210399_x_at | FUT6 | fucosyltransferase 6 (alpha (1,3) | −1.659636895 | 0.002495487 |
| | | fucosyltransferase) | | |
| 1555809_at | CRISPLD2 | cysteine-rich secretory protein LCCL | −1.665011762 | 0.009303531 |
| | | domain containing 2 | | |
| 205778_at | KLK7 | kallikrein 7 (chymotryptic, stratum | −1.67415974 | 0.009247674 |
| | | corneum) | | |
| 210333_at | NR5A1 | nuclear receptor subfamily 5, group A, | −1.684727862 | 1.43E−05 |
| | | member 1 | | |
| 220267_at | KRT24 | keratin 24 | −1.689243936 | 0.002963446 |
| 236617_at | WWOX | WW domain containing oxidoreductase | −1.692626614 | 0.003463158 |
| 1552725_s_at | ADAMTS17 | ADAM metallopeptidase with | −1.711706721 | 0.007309411 |
| | | thrombospondin type 1 motif, 17 | | |
| 1567076_at | — | — | −1.71822618 | 0.006240303 |
| 233234_at | KCTD16 | potassium channel tetramerisation domain | −1.722362172 | 0.006425881 |
| | | containing 16 | | |
| 1566785_x_at | NSF | N-ethylmaleimide-sensitive factor | −1.729464741 | 0.008282015 |
| 243803_at | LOC643037 | hypothetical protein LOC643037 | −1.737080081 | 0.000258191 |
| 208123_at | KCNB2 | potassium voltage-gated channel, Shab- | −1.739091248 | 0.009940012 |
| | | related subfamily, member 2 | | |
| 222265_at | TNS4 | tensin 4 | −1.747764787 | 0.002228676 |
| 207635_s_at | KCNH1 | potassium voltage-gated channel, | −1.754568832 | 8.31E−05 |
| | | subfamily H (eag-related), member 1 | | |
| 229761_at | LOC440173 | LOC440173 | −1.773289766 | 0.006742824 |
| 208383_s_at | PCK1 | phosphoenolpyruvate carboxykinase 1 | −1.781456836 | 0.005690277 |
| | | (soluble) | | |
| 1559025_at | 9-Sep | septin 9 | −1.804776588 | 0.00320637 |
| 203070_at | SEMA3B | sema domain, immunoglobulin domain (Ig), | −1.809960345 | 0.000239576 |
| | | short basic domain, secreted, (semaphorin) | | |
| | | 3B | | |
| 1560745_at | — | CDNA clone IMAGE: 5299239 | −1.812364214 | 0.006413608 |
| 241735_at | — | — | −1.838827522 | 0.001887762 |
| 1566106_at | — | CDNA FLJ33958 fis, clone | −1.874704734 | 1.32E−05 |
| | | CTONG2018735 | | |
| 241503_at | FAM81A | Family with sequence similarity 81, | −1.877466226 | 0.005116259 |
| | | member A | | |
| 224169_at | NPFFR2 | neuropeptide FF receptor 2 | −1.939454807 | 7.47E−05 |
| 243424_at | SOX6 | SRY (sex determining region Y)-box 6 | −1.956385147 | 0.002341982 |
| 240073_at | MSL2L1 | Male-specific lethal 2-like 1 (Drosophila) | −1.973955706 | 0.006568857 |
| 208589_at | TRPC7 | transient receptor potential cation channel, | −2.008819172 | 0.007789466 |
| | | subfamily C, member 7 | | |
| 1555368_x_at | ZNF479 | zinc finger protein 479 | −2.010526838 | 0.008145113 |
| 1562292_at | ANKRD30B | ankyrin repeat domain 30B | −2.135782618 | 0.005388501 |
| 1566865_at | C7orf38 | chromosome 7 open reading frame 38 | −2.139628882 | 8.52E−05 |
| 206922_at | VCY /// VCY1B /// LOC652821 | variable charge, Y-linked /// variable | −2.139677462 | 0.009164238 |
| | | charge, Y-linked 1B /// hypothetical protein | | |
| | | LOC652821 | | |
| 206378_at | SCGB2A2 | secretoglobin, family 2A, member 2 | −2.149229677 | 0.005855605 |
| 233205_at | — | NTera2D1 cell line mRNA containing L1 | −2.227759672 | 0.004494829 |
| | | retroposon, clone P7 | | |
| 1564475_s_at | LOC285711 | hypothetical LOC285711 | −2.229051123 | 0.007050471 |
| 1557215_at | — | CDNA FLJ31650 fis, clone NT2RI2004079 | −2.589961114 | 0.008540571 |
| 1557146_a_at | FLJ32252 | hypothetical protein FLJ32252 | −3.232225882 | 0.005687889 |
| 205979_at | SCGB2A1 | secretoglobin, family 2A, member 1 | −3.602287846 | 0.000888452 |

TABLE 42
dataset 4 (tumor)

| | | | log base | |
|---|---|---|---|---|
| | | | 2 fold | |
| uniqueID | Gene Symbol | Gene Title | change | p value |
| 206509_at | PIP | prolactin-induced protein | 5.370744 | 0.005468 |
| 1557215_at | — | CDNA FLJ31650 fis, clone NT2RI2004079 | 5.223537 | 0.005337 |
| 242601_at | LOC253012 | hypothetical protein LOC253012 | 5.140068 | 3.09E−05 |
| 223642_at | ZIC2 | Zic family member 2 (odd-paired homolog, Drosophila) | 4.91534 | 0.000396 |
| 237384_x_at | — | CDNA clone IMAGE: 5262496 | 4.764076 | 0.000784 |
| 220988_s_at | C1QTNF3 | C1q and tumor necrosis factor related protein 3 /// C1q and tumor | 4.354327 | 1.07E−06 |
| | | necrosis factor related protein 3 | | |
| 242348_at | FAM19A4 | family with sequence similarity 19 (chemokine (C-C motif)-like), | 4.26147 | 3.36E−07 |
| | | member A4 | | |
| 233205_at | — | NTera2D1 cell line mRNA containing L1 retroposon, clone P7 | 4.176793 | 0.000265 |
| 1564358_at | — | CDNA: FLJ22631 fis, clone HSI06451 | 4.148428 | 8.18E−05 |
| 1562527_at | LOC283027 | hypothetical protein LOC283027 | 4.148354 | 0.000141 |
| 210033_s_at | SPAG6 | sperm associated antigen 6 | 4.123292 | 0.004154 |
| 244042_x_at | — | Transcribed locus | 4.120357 | 5.64E−07 |
| 234712_at | C18orf2 | chromosome 18 open reading frame 2 | 3.95219 | 0.000925 |
| 224169_at | NPFFR2 | neuropeptide FF receptor 2 | 3.942727 | 2.19E−06 |
| 1561703_at | — | CDNA clone IMAGE: 5269594 | 3.882851 | 0.003114 |
| 205979_at | SCGB2A1 | secretoglobin, family 2A, member 1 | 3.763634 | 0.004429 |
| 223977_s_at | C18orf2 | chromosome 18 open reading frame 2 | 3.706588 | 0.001951 |
| 1555798_at | DKFZp761D1918 | hypothetical protein DKFZp761D1918 | 3.548908 | 6.45E−05 |
| 234906_at | — | Clone 24900 mRNA sequence | 3.545689 | 0.001437 |
| 222925_at | DCDC2 | doublecortin domain containing 2 | 3.457102 | 0.00331 |
| 215717_s_at | FBN2 | fibrillin 2 (congenital contractural arachnodactyly) | 3.422933 | 0.003768 |
| 204260_at | CHGB | chromogranin B (secretogranin 1) | 3.372877 | 0.003869 |
| 1554591_at | GDEP | Gene differentially expressed in prostate | 3.316701 | 0.006769 |
| 219196_at | SCG3 | secretogranin III | 3.29082 | 0.002324 |
| 237737_at | LOC401131 | hypothetical LOC401131 | 3.279269 | 0.003489 |
| 230493_at | TMEM46 | transmembrane protein 46 | 3.250469 | 0.003163 |
| 1566865_at | C7orf38 | chromosome 7 open reading frame 38 | 3.221464 | 0.000718 |
| 214601_at | TPH1 | tryptophan hydroxylase 1 (tryptophan 5-monooxygenase) | 3.163696 | 0.000148 |
| 235749_at | UGCGL2 | UDP-glucose ceramide glucosyltransferase-like 2 | 2.987957 | 0.005143 |
| 1558982_at | LOC375010 | hypothetical LOC375010 | 2.978955 | 0.004045 |
| 1555560_at | UGCGL2 | UDP-glucose ceramide glucosyltransferase-like 2 | 2.939338 | 8.38E−06 |
| 1559950_at | FAM66C /// | family with sequence similarity 66, member C /// family with sequence | 2.896482 | 0.000934 |
| | FAM66E /// | similarity 66, member E /// hypothetical protein LOC647351 | | |
| | LOC647351 | | | |
| 1559952_x_at | FAM66C /// | family with sequence similarity 66, member C /// family with sequence | 2.799472 | 0.001187 |
| | FAM66E /// | similarity 66, member E /// hypothetical protein LOC647351 | | |
| | LOC647351 | | | |
| 1560745_at | — | CDNA clone IMAGE: 5299239 | 2.738985 | 0.00276 |
| 1554686_at | STAU2 | staufen, RNA binding protein, homolog 2 (Drosophila) | 2.733498 | 8.59E−05 |
| 234445_at | C6orf12 | chromosome 6 open reading frame 12 | 2.727176 | 0.007569 |
| 1564107_at | — | — | 2.712916 | 0.000444 |
| 214774_x_at | TNRC9 | trinucleotide repeat containing 9 | 2.704597 | 0.001603 |
| 207068_at | ZFP37 | zinc finger protein 37 homolog (mouse) | 2.67925 | 7.84E−06 |
| 215108_x_at | TNRC9 | trinucleotide repeat containing 9 | 2.676845 | 0.001055 |
| 214809_at | — | MRNA full length insert cDNA clone EUROIMAGE 1895238 | 2.676827 | 0.006504 |
| 211424_x_at | METTL7A | methyltransferase like 7A | 2.670228 | 0.006068 |
| 243424_at | SOX6 | SRY (sex determining region Y)-box 6 | 2.667164 | 0.006528 |
| 237856_at | RAP1GDS1 | RAP1, GTP-GDP dissociation stimulator 1 | 2.662984 | 0.008821 |
| 240903_at | L3MBTL4 | L(3)mbt-like 4 (Drosophila) | 2.650135 | 0.009291 |
| 1552708_a_at | DUSP19 | dual specificity phosphatase 19 | 2.561307 | 0.003886 |
| 216623_x_at | TNRC9 | trinucleotide repeat containing 9 | 2.524151 | 0.007986 |
| 223785_at | KIAA1794 | KIAA1794 | 2.517603 | 0.000883 |
| 1554328_at | STXBP4 | syntaxin binding protein 4 | 2.517371 | 0.003984 |
| 233234_at | KCTD16 | potassium channel tetramerisation domain containing 16 | 2.516777 | 0.005388 |
| 237160_at | CCDC83 | coiled-coil domain containing 83 | 2.51575 | 0.007401 |
| 1558714_at | ROBO1 | Roundabout, axon guidance receptor, homolog 1 (Drosophila) | 2.49327 | 0.002306 |
| 214942_at | RBM34 | RNA binding motif protein 34 | 2.488861 | 0.001549 |
| 1556429_a_at | WDR67 | WD repeat domain 67 | 2.469192 | 0.000348 |
| 219877_at | ZMAT4 | zinc finger, matrin type 4 | 2.459738 | 0.006082 |
| 1565915_at | ZC3HAV1 | Zinc finger CCCH-type, antiviral 1 | 2.44519 | 0.000764 |
| 1562063_x_at | NBPF1 /// NBPF3 | neuroblastoma breakpoint family, member 1 /// neuroblastoma | 2.418444 | 0.009413 |
| | /// NBPF11 /// | breakpoint family, member 3 /// neuroblastoma breakpoint family, | | |
| | NBPF20 /// | member 11 /// neuroblastoma breakpoint family, member 20 /// | | |
| | NBPF9 /// | neuroblastoma breakpoint family, member 9 /// neuroblastoma | | |
| | NBPF10 /// | breakpoint family, member 10 /// neuroblastoma breakpoint family, | | |
| | NBPF8 | member 8 | | |
| 236725_at | WWC1 | WW, C2 and coiled-coil domain containing 1 | 2.401507 | 2.08E−07 |
| 1559510_at | BHLHB9 | Basic helix-loop-helix domain containing, class B, 9 | 2.396864 | 0.00895 |
| 242283_at | C1orf67 | chromosome 1 open reading frame 67 | 2.367236 | 0.001916 |
| 1554329_x_at | STXBP4 | syntaxin binding protein 4 | 2.36549 | 0.005447 |
| 232850_at | DCDC2 | Doublecortin domain containing 2 | 2.322514 | 0.000127 |
| 217613_at | TMEM144 | transmembrane protein 144 | 2.312429 | 4.69E−05 |
| 207045_at | FLJ20097 | hypothetical protein LOC55610, isoform b | 2.30056 | 0.00652 |
| 241503_at | FAM81A | Family with sequence similarity 81, member A | 2.276681 | 0.00318 |
| 233194_at | STARD13 | START domain containing 13 | 2.273038 | 0.00858 |
| 244188_at | QSER1 | Glutamine and serine rich 1 | 2.253052 | 0.00929 |
| 235676_at | SEC11L3 | SEC11-like 3 (S. cerevisiae) | 2.249791 | 9.72E−05 |
| 1557366_at | KIAA0565 /// | KIAA0565 gene product /// hypothetical protein FLJ36492 | 2.246164 | 0.002822 |
| | FLJ36492 | | | |
| 222322_at | LOC653198 | Similar to phosphodiesterase 4D, cAMP specific | 2.206492 | 0.004101 |
| 1561263_at | C1QTNF3 | C1q and tumor necrosis factor related protein 3 | 2.205998 | 8.94E−06 |
| 1561165_a_at | DEFB108B /// | defensin, beta 108B /// similar to Beta-defensin 108B precursor | 2.201855 | 0.006461 |
| | LOC644407 /// | (Defensin, beta 108B) (Defensin, beta 108) (Beta-defensin 8) (DEFB-8) | | |
| | LOC645413 /// | (BD-8) (hBD-8) /// similar to Beta-defensin 108B precursor (Defensin, | | |
| | LOC645826 /// | beta 108B) (Defensin, beta 108) (Beta-defensin 8) (DEFB-8) (BD-8) | | |
| | LOC652133 | (hBD-8) /// similar to Beta-defensin 108B precursor (Defensin, beta | | |
| | | 108B) (Defensin, beta 108) (Beta-defensin 8) (DEFB-8) (BD-8) (hBD-8) | | |
| | | /// similar to Beta-defensin 108B precursor (Defensin, beta 108B) | | |
| | | (Defensin, beta 108) (Beta-defensin 8) (DEFB-8) (BD-8) (hBD-8) | | |
| 233263_at | MSH3 | MutS homolog 3 (E. coli) | 2.19874 | 0.005964 |
| 244216_at | ESRRG | Estrogen-related receptor gamma | 2.167516 | 0.008322 |
| 1559640_at | ANKFN1 | Ankyrin-repeat and fibronectin type III domain containing 1 | 2.161444 | 0.0022 |
| 213943_at | TWIST1 | twist homolog 1 (acrocephalosyndactyly 3; Saethre-Chotzen syndrome) | 2.156943 | 0.005444 |
| | | (Drosophila) | | |
| 1554618_at | HRBL | HIV-1 Rev binding protein-like | 2.150913 | 2.58E−07 |
| 1568871_at | — | — | 2.09545 | 0.000336 |
| 233724_at | ARNT | aryl hydrocarbon receptor nuclear translocator | 2.084825 | 0.007184 |
| 241735_at | — | — | 2.079085 | 0.000271 |
| 237998_at | C20orf94 | Chromosome 20 open reading frame 94 | 2.056549 | 0.002084 |
| 204665_at | SIKE | suppressor of IKK epsilon | 2.053456 | 0.000398 |
| 208389_s_at | SLC1A2 | solute carrier family 1 (glial high affinity glutamate transporter), member 2 | 2.027265 | 0.000523 |
| 223664_x_at | BCL2L13 | BCL2-like 13 (apoptosis facilitator) | 2.027179 | 0.0007 |
| 240655_at | ALCAM | Activated leukocyte cell adhesion molecule | 2.023936 | 0.001444 |
| 242693_at | CDC2L5 | Cell division cycle 2-like 5 (cholinesterase-related cell division | 2.014603 | 0.003252 |
| | | controller) | | |
| 223908_at | HDAC8 | histone deacetylase 8 | 2.00582 | 4.28E−05 |
| 210634_at | KLHL20 | kelch-like 20 (Drosophila) | 2.004388 | 0.004455 |
| 239970_at | — | Transcribed locus | 1.997871 | 5.89E−05 |
| 1557538_at | THRAP2 | Thyroid hormone receptor associated protein 2 | 1.996282 | 0.001868 |
| 230198_at | WDR37 | WD repeat domain 37 | 1.968825 | 0.000516 |
| 1563614_at | MTBP | Mdm2, transformed 3T3 cell double minute 2, p53 binding protein | 1.964304 | 0.005975 |
| | | (mouse) binding protein, 104 kDa | | |
| 228530_at | RP11-11C5.2 | Similar to RIKEN cDNA 2410129H14 | 1.961534 | 0.000316 |
| 1552761_at | SLC16A11 | solute carrier family 16 (monocarboxylic acid transporters), member 11 | 1.950466 | 0.003934 |
| 208934_s_at | LGALS8 | lectin, galactoside-binding, soluble, 8 (galectin 8) | 1.947612 | 0.007051 |
| 1559025_at | 9-Sep | septin 9 | 1.940382 | 0.004365 |
| 214636_at | CALCB | calcitonin-related polypeptide, beta | 1.937624 | 0.007079 |
| 1553666_at | CCDC34 | coiled-coil domain containing 34 | 1.936705 | 0.004429 |
| 238937_at | ZNF420 | zinc finger protein 420 | 1.931337 | 0.000164 |
| 237483_at | PLEKHA5 | Pleckstrin homology domain containing, family A member 5 | 1.925567 | 0.001273 |
| 1560763_at | DIP2C | DIP2 disco-interacting protein 2 homolog C (Drosophila) | 1.913619 | 0.000588 |
| 1559316_at | LOC646296 | Hypothetical protein LOC646296 | 1.913396 | 0.006315 |
| 1555568_at | MGC22265 | hypothetical protein MGC22265 | 1.910277 | 0.009533 |
| 239706_x_at | FLJ30307 | Hypothetical gene supported by AK054869 | 1.881529 | 0.007808 |
| 229532_at | ZNF502 | zinc finger protein 502 | 1.878032 | 0.000378 |
| 234517_at | — | — | 1.87712 | 0.0055 |
| 240016_at | SENP6 | SUMO1/sentrin specific peptidase 6 | 1.876683 | 0.009473 |
| 231247_s_at | — | CDNA FLJ36837 fis, clone ASTRO2011422 | 1.87431 | 0.000561 |
| 211810_s_at | GALC | galactosylceramidase | 1.864593 | 0.001116 |
| 203491_s_at | CEP57 | centrosomal protein 57 kDa | 1.862976 | 5.92E−05 |
| 1552622_s_at | POLR2J2 /// | DNA directed RNA polymerase II polypeptide J-related gene /// similar | 1.848002 | 0.000155 |
| | LOC441259 | to postmeiotic segregation increased 2-like 2 | | |
| 243226_at | PIN4 | Protein (peptidylprolyl cis/trans isomerase) NIMA-interacting, 4 | 1.846918 | 0.000717 |
| | | (parvulin) | | |
| 204784_s_at | MLF1 | myeloid leukemia factor 1 | 1.842503 | 0.002333 |
| 236314_at | — | Transcribed locus, weakly similar to NP_055301.1 neuronal thread | 1.837609 | 0.001534 |
| | | protein AD7c-NTP [Homo sapiens] | | |
| 1569409_x_at | INHA | Inhibin, alpha | 1.829428 | 0.004667 |
| 219571_s_at | ZNF12 | zinc finger protein 12 | 1.82897 | 0.000993 |
| 1566673_at | LOC90693 | LOC90693 protein | 1.825825 | 0.007215 |
| 1565874_at | KIAA1279 | KIAA1279 | 1.821584 | 0.001597 |
| 234689_at | RP3-402H5.2 | similar to dJ402H5.2 (novel protein similar to worm and fly proteins) | 1.818989 | 0.009595 |
| 1555465_at | MCOLN2 | mucolipin 2 | 1.817584 | 0.005293 |
| 243673_at | RP13-383K5.1 | Hypothetical protein FLJ11016 | 1.810954 | 0.005703 |
| 236987_at | — | — | 1.804452 | 0.002235 |
| 214197_s_at | SETDB1 | SET domain, bifurcated 1 | 1.803863 | 0.001715 |
| 222336_at | LOC201895 | hypothetical protein LOC201895 | 1.798473 | 0.007738 |
| 1560001_at | LOC389634 | Hypothetical LOC389634 | 1.779944 | 0.00383 |
| 1554841_at | MTHFD2L | methylenetetrahydrofolate dehydrogenase (NADP+ dependent) 2-like | 1.777895 | 0.004139 |
| 235172_at | — | Transcribed locus | 1.776562 | 0.000325 |
| 219105_x_at | ORC6L | origin recognition complex, subunit 6 like (yeast) | 1.772037 | 0.004854 |
| 242778_at | LPXN | leupaxin | 1.76512 | 0.004203 |
| 228566_at | P15RS | Hypothetical protein FLJ10656 | 1.763999 | 0.005519 |
| 1569680_at | ZNF704 | Zinc finger protein 704 | 1.753656 | 9.71E−05 |
| 205395_s_at | MRE11A | MRE11 meiotic recombination 11 homolog A (S. cerevisiae) | 1.742194 | 0.007034 |
| 243620_at | RP11-151A6.2 | Hypothetical protein BC004360 | 1.731318 | 0.000127 |
| 1554021_a_at | ZNF12 | zinc finger protein 12 | 1.727005 | 0.001546 |
| 240545_at | LOC286382 | hypothetical protein LOC286382 | 1.722127 | 0.00286 |
| 1568981_at | GTF2I | General transcription factor II, i | 1.707587 | 0.005214 |
| 213989_x_at | SETD4 | SET domain containing 4 | 1.705334 | 0.000365 |
| 230899_at | SETD4 | SET domain containing 4 | 1.704865 | 0.003104 |
| 225066_at | — | — | 1.700769 | 0.009963 |
| 238620_at | SMA3 | SMA3 | 1.698862 | 0.002514 |
| 1555682_at | OTUB2 | OTU domain, ubiquitin aldehyde binding 2 | 1.697097 | 0.003829 |
| 228641_at | CARD8 | caspase recruitment domain family, member 8 | 1.694451 | 0.009231 |
| 236761_at | LHFPL3 | lipoma HMGIC fusion partner-like 3 | 1.689901 | 0.006398 |
| 215170_s_at | CEP152 | centrosomal protein 152 kDa | 1.683443 | 0.001071 |
| 239797_at | CEPT1 | Choline/ethanolamine phosphotransferase 1 | 1.669596 | 0.007754 |
| 207374_at | PLSCR2 | phospholipid scramblase 2 | 1.668642 | 0.00147 |
| 228144_at | ZNF300 | zinc finger protein 300 | 1.667704 | 0.004933 |
| 239815_at | NCOA5 | Nuclear receptor coactivator 5 | 1.666919 | 0.004491 |
| 238358_x_at | DIP2C | DIP2 disco-interacting protein 2 homolog C (Drosophila) | 1.665611 | 0.005407 |
| 236199_at | ALOX5 | Arachidonate 5-lipoxygenase | 1.660315 | 0.007482 |
| 210054_at | C4orf15 | chromosome 4 open reading frame 15 | 1.660275 | 0.001379 |
| 1560638_a_at | — | CDNA clone IMAGE: 5744875 | 1.659382 | 0.003588 |
| 1560705_at | SLC25A28 | Solute carrier family 25, member 28 | 1.656627 | 0.00892 |
| 237118_at | ANP32A | Acidic (leucine-rich) nuclear phosphoprotein 32 family, member A | 1.651692 | 0.008363 |
| 240309_at | — | Transcribed locus | 1.650435 | 0.000939 |
| 1559003_a_at | LOC126661 | hypothetical protein LOC126661 | 1.646021 | 0.002672 |
| 205531_s_at | GLS2 | glutaminase 2 (liver, mitochondrial) | 1.641202 | 0.003665 |
| 219069_at | ANKRD49 | ankyrin repeat domain 49 | 1.632604 | 0.007272 |
| 216451_at | STK38 | Serine/threonine kinase 38 | 1.631083 | 0.002377 |
| 230055_at | C6orf148 | chromosome 6 open reading frame 148 | 1.630245 | 0.005345 |
| 232588_at | STAG1 | stromal antigen 1 | 1.628222 | 0.006896 |
| 242452_at | ABP1 | Amiloride binding protein 1 (amine oxidase (copper-containing)) | 1.627529 | 0.00222 |
| 235890_at | — | Transcribed locus | 1.624147 | 0.005546 |
| 206048_at | OVOL2 | ovo-like 2 (Drosophila) | 1.622425 | 0.001609 |
| 244340_x_at | — | — | 1.621085 | 0.002533 |
| 227384_s_at | LOC644655 | Similar to poly (ADP-ribose) polymerase family, member 8 | 1.617363 | 0.004259 |
| 1557649_at | GALE | UDP-galactose-4-epimerase | 1.615017 | 0.003919 |
| 219871_at | FLJ13197 | hypothetical protein FLJ13197 | 1.612706 | 0.003161 |
| 1558792_x_at | AP2A1 | Adaptor-related protein complex 2, alpha 1 subunit | 1.609076 | 0.009341 |
| 1554501_at | TSC22D4 | TSC22 domain family, member 4 | 1.608186 | 0.000113 |
| 237184_at | ATXN2 | Ataxin 2 | 1.601759 | 0.001539 |
| 237839_at | ANK3 | Ankyrin 3, node of Ranvier (ankyrin G) | 1.599907 | 0.008428 |
| 239044_at | ITFG1 | Integrin alpha FG-GAP repeat containing 1 | 1.597316 | 0.002592 |
| 223538_at | SERF1A | small EDRK-rich factor 1A (telomeric) | 1.595158 | 0.005754 |
| 232928_at | FARP1 | FERM, RhoGEF (ARHGEF) and pleckstrin domain protein 1 | 1.589209 | 0.006714 |
| | | (chondrocyte-derived) | | |
| 235530_at | LOC646040 | Hypothetical protein LOC646040 | 1.583953 | 0.007036 |
| 229050_s_at | MGC16037 | hypothetical protein MGC16037 | 1.582899 | 0.00024 |
| 216419_at | CROCC | ciliary rootlet coiled-coil, rootletin | 1.576767 | 0.00201 |
| 230949_at | SLC23A3 | solute carrier family 23 (nucleobase transporters), member 3 | 1.572954 | 0.005823 |
| 1559870_at | TDH | L-threonine dehydrogenase | 1.570297 | 0.004885 |
| 228403_at | C9orf165 | chromosome 9 open reading frame 165 | 1.569833 | 0.007167 |
| 228981_at | LOC92691 | hypothetical protein BC008604 | 1.569626 | 0.000424 |
| 240555_at | MITF | Microphthalmia-associated transcription factor | 1.568946 | 0.008038 |
| 1565908_at | NADSYN1 | NAD synthetase 1 | 1.562341 | 0.002917 |
| 1559881_s_at | ZNF12 | zinc finger protein 12 | 1.557734 | 0.00827 |
| 232615_at | PDE4DIP | Phosphodiesterase 4D interacting protein (myomegalin) | 1.557032 | 0.0007 |
| 1552621_at | POLR2J2 | DNA directed RNA polymerase II polypeptide J-related gene | 1.555231 | 0.000415 |
| 1560451_at | PKN3 | Protein kinase N3 | 1.554297 | 0.00811 |
| 235983_at | ALS2CR2 | Amyotrophic lateral sclerosis 2 (juvenile) chromosome region, | 1.551939 | 0.008505 |
| | | candidate 2 | | |
| 220673_s_at | KIAA1622 | KIAA1622 | 1.533947 | 0.005511 |
| 213297_at | RMND5B | required for meiotic nuclear division 5 homolog B (S. cerevisiae) | 1.533417 | 0.005142 |
| 243401_at | FMNL2 | Formin-like 2 | 1.526203 | 0.003657 |
| 1557885_at | — | CDNA clone IMAGE: 5277293 | 1.521979 | 0.001459 |
| 218458_at | GMCL1 | germ cell-less homolog 1 (Drosophila) | 1.520215 | 0.000333 |
| 229157_at | ABP1 | Amiloride binding protein 1 (amine oxidase (copper-containing)) | 1.515766 | 0.005735 |
| 213175_s_at | SNRPB | small nuclear ribonucleoprotein polypeptides B and B1 | 1.514534 | 0.000856 |
| 227493_s_at | KIAA1143 /// | KIAA1143 /// KIAA1143 pseudogene | 1.514466 | 0.001349 |
| | LOC644295 | | | |
| 237405_at | — | — | 1.508971 | 0.006542 |
| 243803_at | LOC643037 | hypothetical protein LOC643037 | 1.504271 | 0.001801 |
| 244786_at | C14orf62 | chromosome 14 open reading frame 62 | 1.503168 | 0.002081 |
| 241853_at | — | Transcribed locus | 1.502719 | 0.002701 |
| 223770_x_at | MGC3207 | hypothetical protein MGC3207 | 1.502515 | 0.001335 |
| 243579_at | MSI2 | musashi homolog 2 (Drosophila) | 1.500706 | 0.005754 |
| 230172_at | FAM14B | Family with sequence similarity 14, member B | 1.488063 | 0.000541 |
| 236260_at | — | — | 1.484728 | 0.007 |
| 234495_at | KLK15 | kallikrein 15 | 1.484569 | 0.000942 |
| 214358_at | ACACA | acetyl-Coenzyme A carboxylase alpha | 1.482729 | 0.005085 |
| 1556864_at | FLJ21127 | Tectonic | 1.47985 | 0.00241 |
| 1561906_at | ASIP | Agouti signaling protein, nonagouti homolog (mouse) | 1.479511 | 7.98E−05 |
| 244255_at | LOC286114 | hypothetical protein LOC286114 | 1.474694 | 0.004184 |
| 229636_at | — | — | 1.473429 | 0.003767 |
| 1558527_at | — | CDNA clone IMAGE: 5259766 | 1.471422 | 4.21E−05 |
| 232768_at | CCNB2 | Cyclin B2 | 1.468091 | 0.002596 |
| 240826_at | — | Transcribed locus | 1.468016 | 0.003309 |
| 237942_at | SNRK | SNF related kinase | 1.464326 | 0.005074 |
| 236521_at | — | Transcribed locus | 1.457267 | 0.00372 |
| 213990_s_at | PAK7 | p21(CDKN1A)-activated kinase 7 | 1.456897 | 0.00854 |
| 240507_at | — | CDNA FLJ46224 fis, clone TESTI4014262 | 1.453219 | 0.001915 |
| 237876_at | ITPR2 | Inositol 1,4,5-triphosphate receptor, type 2 | 1.451574 | 0.007403 |
| 207219_at | ZNF643 | zinc finger protein 643 | 1.447976 | 0.00707 |
| 209944_at | ZNF410 | zinc finger protein 410 | 1.446111 | 0.00738 |
| 1558540_s_at | SLC2A11 | solute carrier family 2 (facilitated glucose transporter), member 11 | 1.445914 | 0.004629 |
| 232722_at | RNASET2 | ribonuclease T2 | 1.444273 | 0.006791 |
| 210242_x_at | MTHFS | 5,10-methenyltetrahydrofolate synthetase (5-formyltetrahydrofolate | 1.443608 | 0.004087 |
| | | cyclo-ligase) | | |
| 228009_x_at | ZNRD1 | zinc ribbon domain containing 1 | 1.439903 | 0.003679 |
| 236409_at | LYPLAL1 | Lysophospholipase-like 1 | 1.437865 | 0.006075 |
| 1554303_at | HNMT | histamine N-methyltransferase | 1.431095 | 0.008259 |
| 221510_s_at | GLS | glutaminase | 1.429637 | 0.001693 |
| 1568640_at | LOC400960 | Hypothetical gene supported by BC040598 | 1.429286 | 0.007891 |
| 214355_x_at | LOC441296 | CTAGE family, member 5-like | 1.428373 | 0.006703 |
| 243934_at | — | Transcribed locus | 1.426627 | 0.003559 |
| 230702_at | C8orf16 | chromosome 8 open reading frame 16 | 1.422226 | 0.000706 |
| 228652_at | FLJ38288 | hypothetical protein FLJ38288 | 1.420763 | 0.000243 |
| 213902_at | ASAH1 | N-acylsphingosine amidohydrolase (acid ceramidase) 1 | 1.411323 | 0.000513 |
| 1552809_at | RFX4 | regulatory factor X, 4 (influences HLA class II expression) | 1.410041 | 0.007402 |
| 242966_x_at | ACSBG2 | Acyl-CoA synthetase bubblegum family member 2 | 1.407875 | 0.002887 |
| 219992_at | TAC3 | tachykinin 3 (neuromedin K, neurokinin beta) | 1.392807 | 0.005437 |
| 1438_at | EPHB3 | EPH receptor B3 | 1.392383 | 0.007211 |
| 210884_s_at | LOC653423 | similar to sperm associated antigen 11 isoform H precursor | 1.383915 | 0.004712 |
| 223855_s_at | MCHR1 | melanin-concentrating hormone receptor 1 | 1.380907 | 0.004662 |
| 232216_at | YME1L1 | YME1-like 1 (S. cerevisiae) | 1.379952 | 0.000929 |
| 203657_s_at | CTSF | cathepsin F | 1.37801 | 0.000751 |
| 215816_at | LOC91316 | similar to bK246H3.1 (immunoglobulin lambda-like polypeptide 1, pre- | 1.376229 | 0.002947 |
| | | B-cell specific) | | |
| 242419_at | — | Transcribed locus | 1.374128 | 0.000869 |
| 237995_at | RIMBP2 | RIMS binding protein 2 | 1.370262 | 0.003161 |
| 240120_at | SORBS2 | Sorbin and SH3 domain containing 2 | 1.367839 | 0.000225 |
| 1562630_at | FLJ37543 | Hypothetical protein FLJ37543 | 1.366728 | 0.000206 |
| 201951_at | ALCAM | activated leukocyte cell adhesion molecule | 1.361971 | 0.003251 |
| 211713_x_at | KIAA0101 | KIAA0101 /// KIAA0101 | 1.360682 | 0.001953 |
| 230373_at | LOC644134 | hypothetical protein LOC644134 | 1.356157 | 0.002116 |
| 210763_x_at | NCR3 | natural cytotoxicity triggering receptor 3 | 1.351136 | 7.47E−05 |
| 220058_at | C17orf39 | chromosome 17 open reading frame 39 | 1.347816 | 0.002542 |
| 242035_at | RNF32 | Ring finger protein 32 | 1.332658 | 0.009943 |
| 235274_at | — | Transcribed locus | 1.33222 | 0.008285 |
| 229227_at | FLJ45244 | FLJ45244 protein | 1.331028 | 0.000148 |
| 237750_at | LOC63929 | Hypothetical protein LOC63929 | 1.32525 | 0.003063 |
| 241798_at | SCC-112 | SCC-112 protein | 1.320732 | 0.008326 |
| 222247_at | DXS542 | putative X-linked retinopathy protein | 1.318723 | 0.004166 |
| 1557755_at | C14orf145 | chromosome 14 open reading frame 145 | 1.318397 | 0.003404 |
| 214524_at | GHRH | growth hormone releasing hormone | 1.315693 | 0.009426 |
| 210197_at | ITPK1 | inositol 1,3,4-triphosphate 5/6 kinase | 1.312622 | 0.000972 |
| 237349_at | TSHR | Thyroid stimulating hormone receptor | 1.308968 | 0.004126 |
| 207383_s_at | RHBDL1 | rhomboid, veinlet-like 1 (Drosophila) | 1.303952 | 0.002741 |
| 215420_at | IHH | Indian hedgehog homolog (Drosophila) | 1.303637 | 0.000979 |
| 1565935_at | LOC91431 | prematurely terminated mRNA decay factor-like | 1.302748 | 0.000278 |
| 236766_at | C8orf38 | Chromosome 8 open reading frame 38 | 1.298576 | 0.001 |
| 244547_at | FLJ25006 | Hypothetical protein FLJ25006 | 1.293884 | 0.006825 |
| 243097_x_at | — | Transcribed locus | 1.292402 | 0.008527 |
| 228941_at | — | Transcribed locus | 1.29213 | 0.001012 |
| 237568_at | SLC25A37 | Solute carrier family 25, member 37 | 1.291558 | 0.001012 |
| 1559413_at | TCP11L2 | T-complex 11 (mouse) like 2 | 1.28616 | 0.007173 |
| 1570490_at | — | Homo sapiens, clone IMAGE: 4248504, mRNA | 1.284057 | 0.001737 |
| 217146_at | JRK | jerky homolog (mouse) | 1.275078 | 0.006076 |
| 1560922_s_at | ZNF169 | zinc finger protein 169 | 1.272905 | 0.004609 |
| 227380_x_at | MGC13114 | Hypothetical protein MGC13114 | 1.272591 | 0.001085 |
| 1563452_at | KIAA0241 | KIAA0241 | 1.26728 | 0.004552 |
| 244791_at | PHGDHL1 | Phosphoglycerate dehydrogenase like 1 | 1.263889 | 0.002796 |
| 205796_at | TCP11L1 | t-complex 11 (mouse) like 1 | 1.263141 | 0.00641 |
| 204600_at | EPHB3 | EPH receptor B3 | 1.262703 | 0.002904 |
| 230568_x_at | DLL3 | Delta-like 3 (Drosophila) | 1.261122 | 0.003143 |
| 208828_at | POLE3 | polymerase (DNA directed), epsilon 3 (p17 subunit) | 1.257659 | 0.000792 |
| 214446_at | ELL2 | elongation factor, RNA polymerase II, 2 | 1.251324 | 0.004256 |
| 225315_at | MRPL21 | mitochondrial ribosomal protein L21 | 1.251128 | 0.002753 |
| 209104_s_at | NOLA2 | nucleolar protein family A, member 2 (H/ACA small nucleolar RNPs) | 1.248938 | 0.004515 |
| 1552724_at | OTEX | paired-like homeobox protein OTEX | 1.243882 | 0.002864 |
| 237804_at | DNAH11 | Dynein, axonemal, heavy polypeptide 11 | 1.240909 | 0.007793 |
| 225714_s_at | — | — | 1.238067 | 0.004448 |
| 216690_at | OR7C1 | olfactory receptor, family 7, subfamily C, member 1 | 1.237933 | 0.000297 |
| 1555985_at | C17orf64 | chromosome 17 open reading frame 64 | 1.234519 | 0.007319 |
| 204005_s_at | PAWR | PRKC, apoptosis, WT1, regulator | 1.234501 | 0.008827 |
| 215549_x_at | LOC643854 | similar to CTAGE family, member 5 | 1.227323 | 0.006014 |
| 209939_x_at | CFLAR | CASP8 and FADD-like apoptosis regulator | 1.223319 | 0.004628 |
| 242367_at | — | — | 1.219995 | 0.009172 |
| 238691_at | C14orf62 | chromosome 14 open reading frame 62 | 1.219445 | 0.002765 |
| 211137_s_at | ATP2C1 | ATPase, Ca++ transporting, type 2C, member 1 | 1.218292 | 0.001597 |
| 1553540_a_at | SLC29A2 | solute carrier family 29 (nucleoside transporters), member 2 | 1.21403 | 0.005121 |
| 1554318_at | LOC541473 | FKBP6-like | 1.211465 | 0.005022 |
| 1562799_at | C16orf14 | Chromosome 16 open reading frame 14 | 1.210128 | 0.004706 |
| 219565_at | CYP20A1 | cytochrome P450, family 20, subfamily A, polypeptide 1 | 1.201373 | 0.00921 |
| 241808_at | C8orf70 | Chromosome 8 open reading frame 70 | 1.200515 | 0.007781 |
| 215624_at | TSC2 | Tuberous sclerosis 2 | 1.197729 | 0.004664 |
| 227378_x_at | MGC13114 | hypothetical protein MGC13114 | 1.196386 | 0.000749 |
| 209987_s_at | ASCL1 | achaete-scute complex-like 1 (Drosophila) | 1.195205 | 0.003039 |
| 219595_at | ZNF26 | zinc finger protein 26 | 1.190573 | 0.006675 |
| 221150_at | MEPE | matrix, extracellular phosphoglycoprotein with ASARM motif (bone) | 1.18882 | 0.009576 |
| 1553843_at | C10orf67 | chromosome 10 open reading frame 67 | 1.186881 | 0.008398 |
| 208588_at | FKSG2 | apoptosis inhibitor | 1.184464 | 0.004241 |
| 320_at | PEX6 | peroxisomal biogenesis factor 6 | 1.175461 | 0.006689 |
| 242334_at | NALP4 | NACHT, leucine rich repeat and PYD containing 4 | 1.165243 | 0.006806 |
| 1554480_a_at | SVH | SVH protein | 1.164056 | 0.003106 |
| 212172_at | AK2 | adenylate kinase 2 | 1.162147 | 0.00393 |
| 238354_x_at | DIP2C | DIP2 disco-interacting protein 2 homolog C (Drosophila) | 1.161624 | 0.009309 |
| 233021_at | — | CDNA clone IMAGE: 4825606 | 1.145508 | 0.002336 |
| 208756_at | EIF3S2 | eukaryotic translation initiation factor 3, subunit 2 beta, 36 kDa | 1.141669 | 0.007027 |
| 229232_at | LRRC57 | leucine rich repeat containing 57 | 1.138429 | 0.008867 |
| 232532_at | QRICH2 | glutamine rich 2 | 1.134926 | 0.005514 |
| 220081_x_at | HSD17B7 | hydroxysteroid (17-beta) dehydrogenase 7 | 1.130521 | 0.003678 |
| 207090_x_at | ZFP30 | zinc finger protein 30 homolog (mouse) | 1.129582 | 0.008291 |
| 234041_at | FLJ20054 | Hypothetical protein FLJ20054 | 1.109997 | 0.005575 |
| 215328_at | KIAA0953 | KIAA0953 | 1.100259 | 0.00846 |
| 213222_at | PLCB1 | phospholipase C, beta 1 (phosphoinositide-specific) | 1.091197 | 0.002978 |
| 220583_at | FLJ22596 | hypothetical protein FLJ22596 | 1.090633 | 0.007742 |
| 235994_s_at | SNRPE | Small nuclear ribonucleoprotein polypeptide E | 1.087578 | 0.002384 |
| 221191_at | DKFZP434A0131 | DKFZp434A0131 protein | 1.078814 | 0.001843 |
| 1557736_at | NKTR | natural killer-tumor recognition sequence | 1.076438 | 0.001703 |
| 201782_s_at | AIP | aryl hydrocarbon receptor interacting protein | 1.075471 | 0.00017 |
| 244846_at | MAP4K4 | Mitogen-activated protein kinase kinase kinase kinase 4 | 1.065871 | 0.009863 |
| 234865_at | PRSS1 | Protease, serine, 1 (trypsin 1) | 1.056404 | 0.006638 |
| 238582_at | C21orf2 | Chromosome 21 open reading frame 2 | 1.043799 | 0.003976 |
| 215747_s_at | RCC1 | regulator of chromosome condensation 1 | 1.037827 | 0.002487 |
| 220235_s_at | C1orf103 | chromosome 1 open reading frame 103 | 1.022189 | 0.004537 |
| 216106_at | LOC145678 | hypothetical protein LOC145678 | 1.016043 | 0.003115 |
| 1559261_a_at | PKD1L2 | polycystic kidney disease 1-like 2 | 1.012932 | 0.003293 |
| 1565814_at | TRIM36 | tripartite motif-containing 36 | 1.00602 | 0.008098 |
| 219109_at | SPAG16 | sperm associated antigen 16 | 0.997068 | 0.004838 |
| 212164_at | C1orf37 | chromosome 1 open reading frame 37 | 0.992666 | 0.008557 |
| 215450_at | — | — | 0.987809 | 0.004191 |
| 225298_at | PNKD | paroxysmal nonkinesiogenic dyskinesia | 0.98651 | 0.009537 |
| 1568915_at | — | CDNA clone IMAGE: 4822684 | 0.982944 | 0.000895 |
| 224752_at | LOC641845 /// | hypothetical protein LOC641845 /// hypothetical protein LOC647087 | 0.97834 | 0.004775 |
| | LOC647087 | | | |
| 231189_at | — | — | 0.978193 | 0.00552 |
| 1553262_a_at | UTS2R | urotensin 2 receptor | 0.973296 | 0.003346 |
| 222730_s_at | ZDHHC2 | zinc finger, DHHC-type containing 2 | 0.969733 | 0.00669 |
| 232476_at | DDEF2 | Development and differentiation enhancing factor 2 | 0.958313 | 0.006317 |
| 240099_at | ARSG | Arylsulfatase G | 0.937941 | 0.005798 |
| 232760_at | TEX15 | testis expressed sequence 15 | 0.933436 | 0.002981 |
| 232609_at | CRB3 | crumbs homolog 3 (Drosophila) | 0.927608 | 0.00946 |
| 211728_s_at | HYAL3 | hyaluronoglucosaminidase 3 /// hyaluronoglucosaminidase 3 | 0.922013 | 0.008622 |
| 226466_s_at | FAM58A | family with sequence similarity 58, member A | 0.919631 | 0.008675 |
| 213510_x_at | LOC220594 | TL132 protein | 0.906052 | 0.006946 |
| 218133_s_at | NIF3L1 | NIF3 NGG1 interacting factor 3-like 1 (S. pombe) | 0.882505 | 0.005033 |
| 228114_x_at | MGC13114 | hypothetical protein MGC13114 | 0.877154 | 0.007164 |
| 213112_s_at | SQSTM1 | sequestosome 1 | 0.866737 | 0.002455 |
| 225578_at | RP11-11C5.2 | similar to RIKEN cDNA 2410129H14 | 0.847354 | 0.0047 |
| 206900_x_at | ZNF253 | zinc finger protein 253 | 0.844012 | 0.005518 |
| 236845_at | TRIM62 /// | tripartite motif-containing 62 /// similar to tripartite motif-containing 62 /// | 0.804996 | 0.009157 |
| | LOC653200 /// | similar to tripartite motif-containing 62 | | |
| | LOC654193 | | | |
| 214335_at | RPL18 | ribosomal protein L18 | 0.759396 | 0.003016 |
| 1553257_at | GAL3ST3 | galactose-3-O-sulfotransferase 3 | 0.750273 | 0.006205 |
| 206809_s_at | HNRPA3P1 /// | heterogeneous nuclear ribonucleoprotein A3 pseudogene 1 /// | −0.70998 | 0.009046 |
| | HNRPA3 | heterogeneous nuclear ribonucleoprotein A3 | | |
| 1563139_at | — | — | −0.77574 | 0.00931 |
| 214211_at | FTH1 | ferritin, heavy polypeptide 1 | −0.79786 | 0.009908 |
| 209163_at | CYB561 | cytochrome b-561 | −0.79883 | 0.009637 |
| 211932_at | HNRPA3 | heterogeneous nuclear ribonucleoprotein A3 | −0.83927 | 0.006626 |
| 1556896_at | LOC284751 | hypothetical protein LOC284751 | −0.85723 | 0.007002 |
| 219572_at | CADPS2 | Ca2+-dependent activator protein for secretion 2 | −0.85833 | 0.002441 |
| 237337_at | — | Transcribed locus, moderately similar to XP_510261.1 PREDICTED: | −0.90502 | 0.006938 |
| | | similar to Gamma-tubulin complex component 5 (GCP-5) [Pan troglodytes] | | |
| 243353_at | PDCD8 | Programmed cell death 8 (apoptosis-inducing factor) | −0.9194 | 0.005694 |
| 238761_at | — | — | −0.93593 | 0.008743 |
| 209350_s_at | GPS2 | G protein pathway suppressor 2 | −0.93728 | 0.003127 |
| 204616_at | UCHL3 | ubiquitin carboxyl-terminal esterase L3 (ubiquitin thiolesterase) | −0.93997 | 0.008977 |
| 203027_s_at | MVD | mevalonate (diphospho) decarboxylase | −0.95063 | 0.006188 |
| 224895_at | YAP1 | Yes-associated protein 1, 65 kDa | −0.95499 | 0.006464 |
| 228333_at | — | Full length insert cDNA clone YT94E02 | −0.97206 | 0.007502 |
| 228918_at | SLC43A2 | Solute carrier family 43, member 2 | −0.9798 | 0.0058 |
| 204155_s_at | KIAA0999 | KIAA0999 protein | −1.00003 | 0.002896 |
| 206512_at | U2AF1L1 | U2 small nuclear RNA auxillary factor 1-like 1 | −1.00232 | 0.000939 |
| 223540_at | PVRL4 | poliovirus receptor-related 4 | −1.00537 | 0.005218 |
| 202059_s_at | KPNA1 | karyopherin alpha 1 (importin alpha 5) | −1.0142 | 0.007949 |
| 203514_at | MAP3K3 | mitogen-activated protein kinase kinase kinase 3 | −1.02787 | 0.009559 |
| 212535_at | MEF2A | MADS box transcription enhancer factor 2, polypeptide A (myocyte | −1.04978 | 0.005454 |
| | | enhancer factor 2A) | | |
| 224833_at | ETS1 | v-ets erythroblastosis virus E26 oncogene homolog 1 (avian) | −1.05448 | 0.004297 |
| 228446_at | KIAA2026 | KIAA2026 | −1.05502 | 0.000782 |
| 238006_at | — | Transcribed locus | −1.05537 | 0.008209 |
| 208044_s_at | PPARD | peroxisome proliferative activated receptor, delta | −1.06383 | 0.004705 |
| 215891_s_at | GM2A | GM2 ganglioside activator | −1.07425 | 0.006952 |
| 208806_at | CHD3 | chromodomain helicase DNA binding protein 3 | −1.08149 | 0.005509 |
| 229969_at | — | — | −1.08377 | 0.004459 |
| 242336_at | — | MRNA; cDNA DKFZp686L15210 (from clone DKFZp686L15210) | −1.08536 | 0.000338 |
| 212521_s_at | PDE8A | phosphodiesterase 8A | −1.09953 | 0.007879 |
| 206796_at | WISP1 | WNT1 inducible signaling pathway protein 1 | −1.10378 | 0.000768 |
| 224791_at | DDEF1 | development and differentiation enhancing factor 1 | −1.10479 | 0.006674 |
| 201208_s_at | TNFAIP1 | tumor necrosis factor, alpha-induced protein 1 (endothelial) | −1.10795 | 0.00064 |
| 201536_at | DUSP3 | dual specificity phosphatase 3 (vaccinia virus phosphatase VH1- | −1.11062 | 0.00356 |
| | | related) | | |
| 226865_at | — | MRNA; cDNA DKFZp564O0862 (from clone DKFZp564O0862) | −1.11218 | 0.002939 |
| 222403_at | MTCH2 | mitochondrial carrier homolog 2 (C. elegans) | −1.11298 | 0.00022 |
| 219524_s_at | — | — | −1.12157 | 0.000528 |
| 214856_at | SPTBN1 | spectrin, beta, non-erythrocytic 1 | −1.12671 | 0.004122 |
| 224923_at | TTC7A | tetratricopeptide repeat domain 7A | −1.12767 | 0.005651 |
| 242989_at | — | Transcribed locus | −1.12781 | 0.005934 |
| 203662_s_at | TMOD1 | tropomodulin 1 | −1.13244 | 0.009803 |
| 213306_at | MPDZ | multiple PDZ domain protein | −1.13612 | 0.003665 |
| 203603_s_at | ZFHX1B | zinc finger homeobox 1b | −1.13782 | 0.008641 |
| 227482_at | ADCK1 | aarF domain containing kinase 1 | −1.13841 | 0.008611 |
| 232212_at | PLEKHA8 | pleckstrin homology domain containing, family A (phosphoinositide | −1.14617 | 0.007885 |
| | | binding specific) member 8 | | |
| 201850_at | CAPG | capping protein (actin filament), gelsolin-like | −1.14721 | 0.006793 |
| 206544_x_at | SMARCA2 | SWI/SNF related, matrix associated, actin dependent regulator of | −1.15656 | 0.007813 |
| | | chromatin, subfamily a, member 2 | | |
| 225372_at | C10orf54 | chromosome 10 open reading frame 54 | −1.15903 | 0.003388 |
| 200879_s_at | EPAS1 | endothelial PAS domain protein 1 | −1.16051 | 0.002511 |
| 210139_s_at | PMP22 | peripheral myelin protein 22 | −1.16456 | 0.006819 |
| 214298_x_at | 6-Sep | septin 6 | −1.16815 | 0.008535 |
| 238512_at | ST7L | Suppression of tumorigenicity 7 like | −1.17178 | 0.007905 |
| 205192_at | MAP3K14 | mitogen-activated protein kinase kinase kinase 14 | −1.17286 | 0.004235 |
| 225825_at | C20orf194 | chromosome 20 open reading frame 194 | −1.17302 | 0.008634 |
| 224973_at | FAM46A | Family with sequence similarity 46, member A | −1.17458 | 0.00625 |
| 241840_at | — | Transcribed locus | −1.17765 | 0.009114 |
| 241279_at | PRG1 | Proteoglycan 1, secretory granule | −1.18163 | 0.00181 |
| 238658_at | — | Transcribed locus | −1.18185 | 0.00677 |
| 228471_at | ANKRD44 | Ankyrin repeat domain 44 | −1.19 | 0.005541 |
| 214462_at | SOCS6 | suppressor of cytokine signaling 6 | −1.19019 | 0.002099 |
| 201061_s_at | STOM | stomatin | −1.19277 | 0.006676 |
| 239108_at | MLSTD1 | Male sterility domain containing 1 | −1.19865 | 0.002042 |
| 223380_s_at | LATS2 | LATS, large tumor suppressor, homolog 2 (Drosophila) | −1.20207 | 0.000177 |
| 212339_at | EPB41L1 | erythrocyte membrane protein band 4.1-like 1 | −1.20845 | 0.008039 |
| 219620_x_at | FLJ20245 | hypothetical protein FLJ20245 | −1.20914 | 0.002329 |
| 212235_at | PLXND1 | plexin D1 | −1.21 | 0.004115 |
| 229226_at | MMS19L | MMS19-like (MET18 homolog, S. cerevisiae) | −1.21214 | 0.009982 |
| 210617_at | PHEX | phosphate regulating endopeptidase homolog, X-linked | −1.21394 | 0.004324 |
| | | (hypophosphatemia, vitamin D resistant rickets) | | |
| 210120_s_at | RANBP3 | RAN binding protein 3 | −1.21514 | 0.002636 |
| 242842_at | — | Transcribed locus, strongly similar to NP_071424.1 parvin, gamma; | −1.21569 | 0.006822 |
| | | gamma-parvin [Homo sapiens] | | |
| 213005_s_at | ANKRD15 | ankyrin repeat domain 15 | −1.21579 | 0.008748 |
| 231822_at | CTTNBP2NL | CTTNBP2 N-terminal like | −1.22395 | 0.008873 |
| 205973_at | FEZ1 | fasciculation and elongation protein zeta 1 (zygin I) | −1.23419 | 0.002381 |
| 1553348_a_at | NFX1 | nuclear transcription factor, X-box binding 1 | −1.24115 | 0.007998 |
| 213113_s_at | SLC43A3 | solute carrier family 43, member 3 | −1.24526 | 0.005415 |
| 228819_at | TSPAN18 | tetraspanin 18 | −1.24882 | 0.00781 |
| 224574_at | C17orf49 /// | chromosome 17 open reading frame 49 /// similar to DNA segment, Chr | −1.2558 | 0.002174 |
| | MGC71993 | 11, Brigham & Womens Genetics 0434 expressed | | |
| 207145_at | GDF8 | growth differentiation factor 8 | −1.25828 | 0.009843 |
| 224912_at | TTC7A | tetratricopeptide repeat domain 7A | −1.26224 | 0.004473 |
| 238925_at | — | CDNA clone IMAGE: 5263917 | −1.268 | 0.00839 |
| 230163_at | LOC143381 | Hypothetical protein LOC143381 | −1.27276 | 0.008542 |
| 1564257_at | STAMBPL1 | STAM binding protein-like 1 | −1.27436 | 0.003713 |
| 229068_at | CCT5 | chaperonin containing TCP1, subunit 5 (epsilon) | −1.27903 | 0.006731 |
| 210619_s_at | HYAL1 | hyaluronoglucosaminidase 1 | −1.27999 | 0.00382 |
| 219384_s_at | ADAT1 | adenosine deaminase, tRNA-specific 1 | −1.28034 | 0.009878 |
| 235411_at | PGBD1 | piggyBac transposable element derived 1 | −1.28309 | 0.008675 |
| 202401_s_at | SRF | serum response factor (c-fos serum response element-binding | −1.28463 | 0.002766 |
| | | transcription factor) | | |
| 228526_at | NANOS1 | Nanos homolog 1 (Drosophila) | −1.28761 | 0.005273 |
| 208146_s_at | CPVL | carboxypeptidase, vitellogenic-like /// carboxypeptidase, vitellogenic- | −1.28914 | 0.005301 |
| | | like | | |
| 228935_at | SLC4A8 | solute carrier family 4, sodium bicarbonate cotransporter, member 8 | −1.28949 | 0.000767 |
| 240762_at | — | Transcribed locus | −1.29012 | 0.003178 |
| 202192_s_at | GAS7 | growth arrest-specific 7 | −1.29148 | 0.007392 |
| 236200_at | — | — | −1.29277 | 0.005167 |
| 230348_at | LATS2 | LATS, large tumor suppressor, homolog 2 (Drosophila) | −1.29278 | 0.00842 |
| 210695_s_at | WWOX | WW domain containing oxidoreductase | −1.30383 | 0.009268 |
| 212097_at | CAV1 | caveolin 1, caveolae protein, 22 kDa | −1.3062 | 0.001736 |
| 37152_at | PPARD | peroxisome proliferative activated receptor, delta | −1.30634 | 0.007151 |
| 228120_at | EIF2C1 | Eukaryotic translation initiation factor 2C, 1 | −1.30683 | 0.008055 |
| 224978_s_at | USP36 | ubiquitin specific peptidase 36 | −1.30876 | 0.007345 |
| 201482_at | QSCN6 | quiescin Q6 | −1.31125 | 0.007843 |
| 227013_at | LATS2 | LATS, large tumor suppressor, homolog 2 (Drosophila) | −1.31506 | 0.003851 |
| 208158_s_at | OSBPL1A | oxysterol binding protein-like 1A /// oxysterol binding protein-like 1A | −1.31927 | 0.001846 |
| 209239_at | NFKB1 | nuclear factor of kappa light polypeptide gene enhancer in B-cells 1 | −1.32151 | 0.005911 |
| | | (p105) | | |
| 211976_at | — | CDNA: FLJ22515 fis, clone HRC12122, highly similar to AF052101 | −1.32174 | 0.007791 |
| | | Homo sapiens clone 23872 mRNA sequence | | |
| 244251_at | LCP2 | Lymphocyte cytosolic protein 2 (SH2 domain containing leukocyte | −1.3228 | 0.005576 |
| | | protein of 76 kDa) | | |
| 229596_at | AMDHD1 | amidohydrolase domain containing 1 | −1.32291 | 0.001873 |
| 219341_at | CLN8 | ceroid-lipofuscinosis, neuronal 8 (epilepsy, progressive with mental | −1.32466 | 0.005075 |
| | | retardation) | | |
| 202616_s_at | MECP2 | methyl CpG binding protein 2 (Rett syndrome) | −1.32512 | 0.00803 |
| 232049_at | — | CDNA: FLJ23065 fis, clone LNG04894 | −1.32615 | 0.008662 |
| 240892_at | — | — | −1.3272 | 0.002108 |
| 212386_at | — | CDNA FLJ37747 fis, clone BRHIP2022986 | −1.34052 | 0.007538 |
| 1553917_at | PIP5K3 | phosphatidylinositol-3-phosphate/phosphatidylinositol 5-kinase, type III | −1.34074 | 0.008028 |
| 1556228_a_at | VCPIP1 | Valosin containing protein (p97)/p47 complex interacting protein 1 | −1.34281 | 0.007621 |
| 1557112_a_at | VPS53 | Vacuolar protein sorting 53 (S. cerevisiae) | −1.34445 | 0.008131 |
| 235203_at | — | Homo sapiens, clone IMAGE: 3866695, mRNA | −1.34454 | 0.001617 |
| 222344_at | C5orf13 | Chromosome 5 open reading frame 13 | −1.34515 | 0.006349 |
| 218094_s_at | DBNDD2 | dysbindin (dystrobrevin binding protein 1) domain containing 2 | −1.34537 | 0.003391 |
| 217890_s_at | PARVA | parvin, alpha | −1.34542 | 0.004315 |
| 228770_at | GPR146 | G protein-coupled receptor 146 | −1.34632 | 0.004782 |
| 224991_at | CMIP | c-Maf-inducing protein | −1.34999 | 0.000122 |
| 202816_s_at | SS18 | synovial sarcoma translocation, chromosome 18 | −1.35172 | 1.47E−06 |
| 203640_at | MBNL2 | muscleblind-like 2 (Drosophila) | −1.35491 | 0.005326 |
| 226121_at | MGC23280 | hypothetical protein MGC23280 | −1.35491 | 0.001453 |
| 203408_s_at | SATB1 | special AT-rich sequence binding protein 1 (binds to nuclear | −1.3596 | 0.004106 |
| | | matrix/scaffold-associating DNA's) | | |
| 215116_s_at | DNM1 | dynamin 1 | −1.35985 | 0.004479 |
| 209539_at | ARHGEF6 | Rac/Cdc42 guanine nucleotide exchange factor (GEF) 6 | −1.36052 | 0.000537 |
| 209485_s_at | OSBPL1A | oxysterol binding protein-like 1A | −1.36085 | 3.82E−05 |
| 204249_s_at | LMO2 | LIM domain only 2 (rhombotin-like 1) | −1.36177 | 0.000854 |
| 238479_at | — | Full length insert cDNA clone ZC34E11 | −1.37061 | 0.009321 |
| 228188_at | — | — | −1.3717 | 0.008321 |
| 1565638_at | PMP22 | Peripheral myelin protein 22 | −1.37243 | 0.009347 |
| 211535_s_at | FGFR1 | fibroblast growth factor receptor 1 (fms-related tyrosine kinase 2, | −1.37321 | 0.007731 |
| | | Pfeiffer syndrome) | | |
| 205112_at | PLCE1 | phospholipase C, epsilon 1 | −1.37328 | 0.003634 |
| 213519_s_at | LAMA2 | laminin, alpha 2 (merosin, congenital muscular dystrophy) | −1.37479 | 0.002355 |
| 1558733_at | ZBTB38 | zinc finger and BTB domain containing 38 | −1.37539 | 0.005994 |
| 219325_s_at | ELAC1 | elaC homolog 1 (E. coli) | −1.37543 | 0.005658 |
| 213548_s_at | CDV3 | CDV3 homolog (mouse) | −1.37995 | 0.005688 |
| 219874_at | SLC12A8 | solute carrier family 12 (potassium/chloride transporters), member 8 | −1.38776 | 0.000826 |
| 239749_at | FAF1 | Fas (TNFRSF6) associated factor 1 | −1.38788 | 0.008796 |
| 223741_s_at | TTYH2 | tweety homolog 2 (Drosophila) | −1.39009 | 0.003939 |
| 208891_at | DUSP6 | dual specificity phosphatase 6 | −1.39211 | 0.004091 |
| 204181_s_at | ZBTB43 | zinc finger and BTB domain containing 43 | −1.39252 | 0.005213 |
| 227138_at | CRTAP | cartilage associated protein | −1.39373 | 0.001976 |
| 217629_at | GNGT2 | Guanine nucleotide binding protein (G protein), gamma transducing | −1.39625 | 0.000937 |
| | | activity polypeptide 2 | | |
| 207749_s_at | PPP2R3A | protein phosphatase 2 (formerly 2A), regulatory subunit B″, alpha | −1.39796 | 0.000569 |
| 239294_at | — | Transcribed locus | −1.40025 | 0.007714 |
| 235637_s_at | — | CDNA FLJ23896 fis, clone LNG15157 | −1.40165 | 0.000335 |
| 210933_s_at | FSCN1 | fascin homolog 1, actin-bundling protein (Strongylocentrotus | −1.40201 | 8.75E−05 |
| | | purpuratus) | | |
| 205841_at | JAK2 | Janus kinase 2 (a protein tyrosine kinase) | −1.40754 | 0.007878 |
| 219777_at | GIMAP6 | GTPase, IMAP family member 6 | −1.41005 | 0.000485 |
| 215427_s_at | ZCCHC14 | zinc finger, CCHC domain containing 14 | −1.41021 | 0.009449 |
| 229461_x_at | NEGR1 | neuronal growth regulator 1 | −1.41879 | 0.009473 |
| 206955_at | AQP7 | aquaporin 7 | −1.42015 | 0.00945 |
| 230240_at | DYRK3 | Dual-specificity tyrosine-(Y)-phosphorylation regulated kinase 3 | −1.42509 | 0.007094 |
| 217585_at | NEBL | nebulette | −1.42535 | 0.003899 |
| 239698_at | — | Transcribed locus | −1.42628 | 0.007096 |
| 203810_at | DNAJB4 | DnaJ (Hsp40) homolog, subfamily B, member 4 | −1.42971 | 0.00775 |
| 232591_s_at | TMEM30A | transmembrane protein 30A | −1.43002 | 0.007571 |
| 38671_at | PLXND1 | plexin D1 | −1.43288 | 0.002925 |
| 215016_x_at | DST | dystonin | −1.43431 | 0.007207 |
| 203002_at | AMOTL2 | angiomotin like 2 | −1.43583 | 0.000621 |
| 205902_at | KCNN3 | potassium intermediate/small conductance calcium-activated channel, | −1.43775 | 0.004648 |
| | | subfamily N, member 3 | | |
| 227367_at | SLCO3A1 | Solute carrier organic anion transporter family, member 3A1 | −1.43806 | 0.001163 |
| 224996_at | MGC34646 | Hypothetical protein MGC34646 | −1.43894 | 0.000983 |
| 226607_at | C20orf194 | chromosome 20 open reading frame 194 | −1.44109 | 0.00407 |
| 202921_s_at | ANK2 | ankyrin 2, neuronal | −1.44169 | 0.001192 |
| 208981_at | PECAM1 | platelet/endothelial cell adhesion molecule (CD31 antigen) | −1.44456 | 0.001204 |
| 200953_s_at | CCND2 | cyclin D2 | −1.44474 | 0.003254 |
| 205116_at | LAMA2 | laminin, alpha 2 (merosin, congenital muscular dystrophy) | −1.44503 | 0.008615 |
| 239548_at | NEGR1 | neuronal growth regulator 1 | −1.45043 | 0.000157 |
| 1560305_x_at | — | CDNA: FLJ21041 fis, clone CAE10652 | −1.45428 | 0.00396 |
| 218711_s_at | SDPR | serum deprivation response (phosphatidylserine binding protein) | −1.45466 | 0.004037 |
| 231823_s_at | SH3PXD2B | SH3 and PX domains 2B | −1.45495 | 0.003441 |
| 1554624_a_at | SIRPB1 | signal-regulatory protein beta 1 | −1.45978 | 0.007602 |
| 230383_x_at | — | Transcribed locus, moderately similar to NP_060190.1 signal- | −1.46094 | 0.001941 |
| | | transducing adaptor protein-2; brk kinase substrate [Homo sapiens] | | |
| 213426_s_at | CAV2 | caveolin 2 | −1.46273 | 0.001997 |
| 233820_at | FAS | Fas (TNF receptor superfamily, member 6) | −1.46325 | 0.004042 |
| 240862_at | RASGRP4 | RAS guanyl releasing protein 4 | −1.46444 | 0.000983 |
| 242414_at | QPRT | quinolinate phosphoribosyltransferase (nicotinate-nucleotide | −1.46535 | 0.004156 |
| | | pyrophosphorylase (carboxylating)) | | |
| 1554360_at | FCHSD2 | FCH and double SH3 domains 2 | −1.4713 | 0.0061 |
| 231867_at | ODZ2 | odz, odd Oz/ten-m homolog 2 (Drosophila) | −1.47236 | 0.005968 |
| 232950_s_at | PITPNM2 | phosphatidylinositol transfer protein, membrane-associated 2 | −1.47856 | 0.004077 |
| 227404_s_at | EGR1 | Early growth response 1 | −1.47863 | 0.008376 |
| 203490_at | ELF4 | E74-like factor 4 (ets domain transcription factor) | −1.47957 | 0.002925 |
| 227882_at | FKRP | fukutin related protein | −1.4802 | 0.001035 |
| 243172_at | — | — | −1.48533 | 0.007311 |
| 235821_at | — | CDNA FLJ14388 fis, clone HEMBA1002716 | −1.48566 | 0.007772 |
| 226774_at | KIAA1838 | KIAA1838 | −1.49019 | 0.001891 |
| 204642_at | EDG1 | endothelial differentiation, sphingolipid G-protein-coupled receptor, 1 | −1.49258 | 0.006837 |
| 213260_at | — | CDNA FLJ11796 fis, clone HEMBA1006158, highly similar to Homo | −1.49301 | 0.008272 |
| | | sapiens transcription factor forkhead-like 7 (FKHL7) gene | | |
| 229936_at | GFRA3 | GDNF family receptor alpha 3 | −1.49409 | 0.00808 |
| 212658_at | LHFPL2 | lipoma HMGIC fusion partner-like 2 | −1.49459 | 0.004496 |
| 212419_at | C10orf56 | chromosome 10 open reading frame 56 | −1.49477 | 0.00085 |
| 213012_at | NEDD4 | neural precursor cell expressed, developmentally down-regulated 4 | −1.49809 | 0.003649 |
| 203688_at | PKD2 | polycystic kidney disease 2 (autosomal dominant) | −1.50099 | 0.006196 |
| 206210_s_at | CETP | cholesteryl ester transfer protein, plasma | −1.50199 | 0.007025 |
| 235252_at | KSR1 | Kinase suppressor of ras 1 | −1.50199 | 0.004945 |
| 205846_at | PTPRB | protein tyrosine phosphatase, receptor type, B | −1.50241 | 0.000777 |
| 213381_at | C10orf72 | Chromosome 10 open reading frame 72 | −1.5029 | 0.006261 |
| 225159_s_at | — | — | −1.50446 | 0.001313 |
| 1560303_at | — | CDNA: FLJ21041 fis, clone CAE10652 | −1.50467 | 0.007752 |
| 230321_at | — | — | −1.50474 | 0.009015 |
| 230750_at | — | Transcribed locus | −1.50952 | 0.006026 |
| 1555923_a_at | C10orf114 | chromosome 10 open reading frame 114 | −1.51035 | 0.008111 |
| 229367_s_at | GIMAP6 | GTPase, IMAP family member 6 | −1.51049 | 0.000468 |
| 226713_at | CCDC50 | coiled-coil domain containing 50 | −1.51078 | 0.00092 |
| 227009_at | — | MRNA; cDNA DKFZp667E0114 (from clone DKFZp667E0114) | −1.51086 | 0.003841 |
| 1556414_at | C21orf71 | chromosome 21 open reading frame 71 | −1.51538 | 0.009659 |
| 232009_at | EMR2 | egf-like module containing, mucin-like, hormone receptor-like 2 | −1.51693 | 0.000524 |
| 244294_at | — | CDNA FLJ26260 fis, clone DMC05193 | −1.51909 | 0.000306 |
| 236395_at | MEF2C | MADS box transcription enhancer factor 2, polypeptide C (myocyte | −1.51927 | 0.009487 |
| | | enhancer factor 2C) | | |
| 208609_s_at | TNXA /// TNXB | tenascin XA pseudogene /// tenascin XA pseudogene /// tenascin XB /// | −1.5198 | 0.000553 |
| | | tenascin XB | | |
| 221016_s_at | TCF7L1 | transcription factor 7-like 1 (T-cell specific, HMG-box) /// transcription | −1.52418 | 0.005948 |
| | | factor 7-like 1 (T-cell specific, HMG-box) | | |
| 242380_at | CCT8 | Chaperonin containing TCP1, subunit 8 (theta) | −1.52573 | 0.005533 |
| 1556314_a_at | — | (clone 1NIB-4) normalized cDNA library sequence | −1.52594 | 0.005521 |
| 212605_s_at | — | AF034176 Human mRNA (Tripodis and Ragoussis) Homo sapiens | −1.52727 | 0.001314 |
| | | cDNA clone ntcon5 contig | | |
| 216033_s_at | FYN | FYN oncogene related to SRC, FGR, YES | −1.53054 | 0.0022 |
| 222054_at | LOC664727 | hypothetical protein LOC664727 | −1.53091 | 0.002407 |
| 216684_s_at | SS18 | synovial sarcoma translocation, chromosome 18 | −1.53479 | 0.005612 |
| 229487_at | — | CDNA FLJ39389 fis, clone PLACE6003621 | −1.53704 | 0.001972 |
| 209687_at | CXCL12 | chemokine (C—X—C motif) ligand 12 (stromal cell-derived factor 1) | −1.53965 | 0.000673 |
| 202007_at | NID1 | nidogen 1 | −1.54043 | 0.008624 |
| 219892_at | TM6SF1 | transmembrane 6 superfamily member 1 | −1.54188 | 0.00737 |
| 232544_at | LOC255130 | Hypothetical LOC255130 | −1.54319 | 0.001504 |
| 227401_at | IL17D | interleukin 17D | −1.54406 | 0.009818 |
| 1557475_at | — | CDNA clone IMAGE: 5274197 | −1.5465 | 0.002529 |
| 225328_at | — | CDNA FLJ39585 fis, clone SKMUS2006633 | −1.54855 | 0.008682 |
| 236388_at | — | — | −1.55091 | 0.005618 |
| 201702_s_at | PPP1R10 | protein phosphatase 1, regulatory subunit 10 | −1.55391 | 0.005372 |
| 227021_at | AOF1 | amine oxidase (flavin containing) domain 1 | −1.55465 | 0.008284 |
| 214337_at | COPA | coatomer protein complex, subunit alpha | −1.55571 | 0.004561 |
| 41577_at | PPP1R16B | protein phosphatase 1, regulatory (inhibitor) subunit 16B | −1.55576 | 0.002048 |
| 212762_s_at | TCF7L2 | transcription factor 7-like 2 (T-cell specific, HMG-box) | −1.55576 | 0.003668 |
| 206465_at | ACSBG1 | acyl-CoA synthetase bubblegum family member 1 | −1.55771 | 0.004278 |
| 209409_at | GRB10 | growth factor receptor-bound protein 10 | −1.55812 | 6.57E−06 |
| 232109_at | UBXD3 | UBX domain containing 3 | −1.55875 | 0.006296 |
| 221447_s_at | GLT8D2 | glycosyltransferase 8 domain containing 2 /// glycosyltransferase 8 | −1.56136 | 0.003641 |
| | | domain containing 2 | | |
| 209740_s_at | PNPLA4 | patatin-like phospholipase domain containing 4 | −1.56246 | 5.92E−05 |
| 233555_s_at | SULF2 | sulfatase 2 | −1.56377 | 0.001817 |
| 208892_s_at | DUSP6 | dual specificity phosphatase 6 | −1.56521 | 0.000386 |
| 212608_s_at | — | AF034176 Human mRNA (Tripodis and Ragoussis) Homo sapiens | −1.56532 | 0.003272 |
| | | cDNA clone ntcon5 contig | | |
| 209086_x_at | MCAM | melanoma cell adhesion molecule | −1.56674 | 0.002682 |
| 212254_s_at | DST | dystonin | −1.56692 | 0.008518 |
| 229667_s_at | HOXB8 | homeobox B8 | −1.5678 | 0.002806 |
| 217442_at | IGSF4B | Immunoglobulin superfamily, member 4B | −1.56902 | 0.000307 |
| 208944_at | TGFBR2 | transforming growth factor, beta receptor II (70/80 kDa) | −1.56989 | 0.000444 |
| 205866_at | FCN3 | ficolin (collagen/fibrinogen domain containing) 3 (Hakata antigen) | −1.57423 | 0.00129 |
| 230523_at | — | — | −1.57452 | 0.005381 |
| 209712_at | SLC35D1 | solute carrier family 35 (UDP-glucuronic acid/UDP-N- | −1.5761 | 0.001902 |
| | | acetylgalactosamine dual transporter), member D1 | | |
| 234033_at | RAPGEF2 | Rap guanine nucleotide exchange factor (GEF) 2 | −1.57661 | 0.001498 |
| 203143_s_at | KIAA0040 | KIAA0040 | −1.57746 | 0.007289 |
| 1563273_at | — | Full length insert cDNA clone ZA89G05 | −1.57829 | 0.00922 |
| 1569052_at | IGF2R | Insulin-like growth factor 2 receptor | −1.57862 | 0.000133 |
| 228103_s_at | NRP2 | Neuropilin 2 | −1.58081 | 0.000196 |
| 237311_at | DOCK1 | Dedicator of cytokinesis 1 | −1.58155 | 0.005522 |
| 219026_s_at | RASAL2 | RAS protein activator like 2 | −1.58247 | 0.001979 |
| 225225_at | KRTAP4-7 | Keratin associated protein 4-7 | −1.58442 | 0.007862 |
| 202156_s_at | CUGBP2 | CUG triplet repeat, RNA binding protein 2 | −1.58487 | 0.001088 |
| 238151_at | TUBB6 | Tubulin, beta 6 | −1.58744 | 0.001874 |
| 239058_at | FOXC2 | Forkhead box C2 (MFH-1, mesenchyme forkhead 1) | −1.58752 | 0.000452 |
| 238025_at | MLKL | mixed lineage kinase domain-like | −1.58769 | 0.006112 |
| 229687_s_at | — | Full length insert cDNA clone ZD53C10 | −1.58772 | 0.007178 |
| 205159_at | CSF2RB | colony stimulating factor 2 receptor, beta, low-affinity (granulocyte- | −1.58827 | 0.002438 |
| | | macrophage) /// colony stimulating factor 2 receptor, beta, low-affinity | | |
| | | (granulocyte-macrophage) | | |
| 243322_at | DPYSL3 | Dihydropyrimidinase-like 3 | −1.58871 | 0.006937 |
| 226950_at | — | CDNA FLJ32163 fis, clone PLACE6000371 | −1.58928 | 0.000248 |
| 228436_at | KCNC4 | potassium voltage-gated channel, Shaw-related subfamily, member 4 | −1.58954 | 0.00273 |
| 229504_at | — | Transcribed locus | −1.59 | 0.002395 |
| 240418_at | — | Transcribed locus | −1.59007 | 0.005357 |
| 228877_at | RGL3 | ral guanine nucleotide dissociation stimulator-like 3 | −1.59213 | 0.005548 |
| 204236_at | FLI1 | Friend leukemia virus integration 1 | −1.59233 | 0.003555 |
| 219295_s_at | PCOLCE2 | procollagen C-endopeptidase enhancer 2 | −1.59369 | 0.006799 |
| 1553453_at | ASB14 | ankyrin repeat and SOCS box-containing 14 | −1.59486 | 0.008419 |
| 228837_at | — | CDNA FLJ37747 fis, clone BRHIP2022986 | −1.59674 | 0.000417 |
| 241300_at | — | — | −1.59699 | 0.006461 |
| 202206_at | ARL4C | ADP-ribosylation factor-like 4C | −1.59779 | 0.004581 |
| 203632_s_at | GPRC5B | G protein-coupled receptor, family C, group 5, member B | −1.59833 | 0.003954 |
| 204845_s_at | ENPEP | glutamyl aminopeptidase (aminopeptidase A) | −1.59883 | 0.002097 |
| 228618_at | PEAR1 | platelet endothelial aggregation receptor 1 | −1.6009 | 0.000556 |
| 209839_at | DNM3 | dynamin 3 | −1.60109 | 0.004655 |
| 209676_at | TFPI | tissue factor pathway inhibitor (lipoprotein-associated coagulation | −1.60896 | 0.003292 |
| | | inhibitor) | | |
| 228857_at | LOC285831 | hypothetical protein LOC285831 | −1.61284 | 0.001505 |
| 209209_s_at | PLEKHC1 | pleckstrin homology domain containing, family C (with FERM domain) | −1.61409 | 0.001742 |
| | | member 1 | | |
| 228748_at | CD59 | CD59 molecule, complement regulatory protein | −1.61654 | 0.003508 |
| 232539_at | — | MRNA; cDNA DKFZp761H1023 (from clone DKFZp761H1023) | −1.62007 | 0.007942 |
| 243735_at | — | CDNA FLJ38461 fis, clone FEBRA2020977 | −1.62112 | 0.007155 |
| 218864_at | TNS1 | tensin 1 | −1.62433 | 0.009092 |
| 205860_x_at | FOLH1 | folate hydrolase (prostate-specific membrane antigen) 1 | −1.62748 | 0.001505 |
| 210077_s_at | SFRS5 | splicing factor, arginine/serine-rich 5 | −1.63195 | 0.001056 |
| 1558517_s_at | LRRC8C | Leucine rich repeat containing 8 family, member C | −1.63209 | 0.000251 |
| 232531_at | EMX2OS | empty spiracles homolog 2 (Drosophila) opposite strand | −1.6329 | 0.004228 |
| 229686_at | P2RY8 | purinergic receptor P2Y, G-protein coupled, 8 | −1.63431 | 0.007307 |
| 215363_x_at | FOLH1 | folate hydrolase (prostate-specific membrane antigen) 1 | −1.63438 | 0.001463 |
| 231202_at | ALDH1L2 | aldehyde dehydrogenase 1 family, member L2 | −1.63557 | 0.007303 |
| 212672_at | ATM | ataxia telangiectasia mutated (includes complementation groups A, C | −1.63671 | 0.001197 |
| | | and D) | | |
| 213451_x_at | TNXB | tenascin XB | −1.63802 | 0.00986 |
| 238704_at | — | Homo sapiens, clone IMAGE: 3866695, mRNA | −1.63819 | 0.009422 |
| 207594_s_at | SYNJ1 | synaptojanin 1 | −1.63841 | 0.008571 |
| 227317_at | LMCD1 | LIM and cysteine-rich domains 1 | −1.63929 | 0.001582 |
| 231945_at | FILIP1 | filamin A interacting protein 1 | −1.64052 | 0.001658 |
| 1553613_s_at | FOXC1 | forkhead box C1 | −1.64054 | 0.006145 |
| 226751_at | C2orf32 | chromosome 2 open reading frame 32 | −1.64089 | 0.005557 |
| 201578_at | PODXL | podocalyxin-like | −1.64098 | 0.000855 |
| 212190_at | SERPINE2 | serpin peptidase inhibitor, clade E (nexin, plasminogen activator | −1.64229 | 0.0092 |
| | | inhibitor type 1), member 2 | | |
| 230057_at | LOC285178 | hypothetical protein LOC285178 | −1.64468 | 0.004494 |
| 205638_at | BAI3 | brain-specific angiogenesis inhibitor 3 | −1.64509 | 0.001332 |
| 244130_at | HTR2A | 5-hydroxytryptamine (serotonin) receptor 2A | −1.646 | 0.005149 |
| 231367_s_at | LOC647131 /// | hypothetical protein LOC647131 /// hypothetical protein LOC652225 | −1.64678 | 0.006446 |
| | LOC652225 | | | |
| 225355_at | DKFZP761M1511 | hypothetical protein DKFZP761M1511 | −1.64873 | 0.001447 |
| 235276_at | — | — | −1.65 | 0.00185 |
| 204963_at | SSPN | sarcospan (Kras oncogene-associated gene) | −1.65373 | 0.006574 |
| 216333_x_at | TNXB | tenascin XB | −1.65404 | 0.007582 |
| 218975_at | COL5A3 | collagen, type V, alpha 3 | −1.65768 | 0.000746 |
| 236154_at | — | CDNA FLJ39382 fis, clone PERIC2000473 | −1.6578 | 0.008876 |
| 210105_s_at | FYN | FYN oncogene related to SRC, FGR, YES | −1.65887 | 0.000341 |
| 219652_s_at | CXorf36 | chromosome X open reading frame 36 | −1.65935 | 0.007321 |
| 238186_at | — | Transcribed locus | −1.66067 | 0.006217 |
| 235560_at | — | MRNA; cDNA DKFZp547A0515 (from clone DKFZp547A0515) | −1.66082 | 0.009471 |
| 233324_at | TRERF1 | transcriptional regulating factor 1 | −1.66106 | 0.001691 |
| 205504_at | BTK | Bruton agammaglobulinemia tyrosine kinase | −1.66661 | 0.004809 |
| 219334_s_at | OBFC2A | oligonucleotide/oligosaccharide-binding fold containing 2A | −1.66719 | 0.007557 |
| 206526_at | RIBC2 | RIB43A domain with coiled-coils 2 | −1.66846 | 0.008426 |
| 223952_x_at | DHRS9 | dehydrogenase/reductase (SDR family) member 9 | −1.6712 | 0.007688 |
| 235723_at | BNC2 | basonuclin 2 | −1.67529 | 0.00288 |
| 207695_s_at | IGSF1 | immunoglobulin superfamily, member 1 | −1.67645 | 0.002278 |
| 230447_at | — | CDNA FLJ30539 fis, clone BRAWH2001255 | −1.67845 | 0.009406 |
| 236359_at | SCN4B | sodium channel, voltage-gated, type IV, beta | −1.67885 | 0.002868 |
| 37005_at | NBL1 | neuroblastoma, suppression of tumorigenicity 1 | −1.67978 | 0.000262 |
| 236945_at | — | — | −1.68001 | 0.003442 |
| 206625_at | RDS | retinal degeneration, slow | −1.68446 | 0.009445 |
| 205822_s_at | HMGCS1 | 3-hydroxy-3-methylglutaryl-Coenzyme A synthase 1 (soluble) | −1.68506 | 0.003579 |
| 228308_at | ARF3 | ADP-ribosylation factor 3 | −1.6859 | 0.00054 |
| 1559007_s_at | — | CDNA clone IMAGE: 4304686 | −1.68703 | 0.008128 |
| 235155_at | BDH2 | 3-hydroxybutyrate dehydrogenase, type 2 | −1.6881 | 0.004417 |
| 232187_at | PALMD | palmdelphin | −1.68872 | 0.000609 |
| 213666_at | 6-Sep | septin 6 | −1.69034 | 0.008113 |
| 225895_at | SYNPO2 | synaptopodin 2 | −1.69087 | 0.005947 |
| 211492_s_at | ADRA1A | adrenergic, alpha-1A-, receptor | −1.69209 | 0.003856 |
| 228255_at | ALS2CR4 | amyotrophic lateral sclerosis 2 (juvenile) chromosome region, | −1.693 | 0.007618 |
| | | candidate 4 | | |
| 209711_at | SLC35D1 | solute carrier family 35 (UDP-glucuronic acid/UDP-N- | −1.69342 | 0.001812 |
| | | acetylgalactosamine dual transporter), member D1 | | |
| 240189_at | ACOXL | Acyl-Coenzyme A oxidase-like | −1.69354 | 0.002803 |
| 227976_at | LOC644538 | hypothetical protein LOC644538 | −1.69468 | 0.003241 |
| 201150_s_at | TIMP3 | TIMP metallopeptidase inhibitor 3 (Sorsby fundus dystrophy, | −1.69548 | 0.000478 |
| | | pseudoinflammatory) | | |
| 244825_at | RP11-119E20.1 | KIAA1202 protein | −1.69685 | 0.001108 |
| 230234_at | — | Transcribed locus, moderately similar to NP_055301.1 neuronal thread | −1.70226 | 0.009247 |
| | | protein AD7c-NTP [Homo sapiens] | | |
| 238577_s_at | — | Transcribed locus | −1.70586 | 0.006341 |
| 234888_at | CACHD1 | Cache domain containing 1 | −1.70855 | 0.002537 |
| 207332_s_at | TFRC | transferrin receptor (p90, CD71) | −1.70928 | 0.005063 |
| 1568760_at | MYH11 | myosin, heavy polypeptide 11, smooth muscle | −1.70973 | 0.005513 |
| 235989_at | — | Transcribed locus | −1.71119 | 0.000193 |
| 239201_at | ALS2CR7 | amyotrophic lateral sclerosis 2 (juvenile) chromosome region, | −1.71301 | 0.000906 |
| | | candidate 7 | | |
| 237204_at | DPH1 | DPH1 homolog (S. cerevisiae) | −1.7142 | 0.007756 |
| 212486_s_at | FYN | FYN oncogene related to SRC, FGR, YES | −1.71651 | 1.16E−05 |
| 204546_at | KIAA0513 | KIAA0513 | −1.7168 | 3.18E−05 |
| 231086_at | BACE1 | Beta-site APP-cleaving enzyme 1 | −1.71718 | 0.006314 |
| 206337_at | CCR7 | chemokine (C-C motif) receptor 7 /// chemokine (C-C motif) receptor 7 | −1.71755 | 0.006686 |
| 219643_at | LRP1B | low density lipoprotein-related protein 1B (deleted in tumors) | −1.71759 | 0.007452 |
| 239236_at | CLIC2 | Chloride intracellular channel 2 | −1.71842 | 0.004739 |
| 211105_s_at | NFATC1 | nuclear factor of activated T-cells, cytoplasmic, calcineurin-dependent 1 | −1.71894 | 0.001493 |
| 201621_at | NBL1 | neuroblastoma, suppression of tumorigenicity 1 | −1.71902 | 0.000547 |
| 230836_at | ST8SIA4 | ST8 alpha-N-acetyl-neuraminide alpha-2,8-sialyltransferase 4 | −1.71966 | 0.007501 |
| 238520_at | — | MSTP020 (MST020) | −1.72093 | 0.00802 |
| 224009_x_at | DHRS9 | dehydrogenase/reductase (SDR family) member 9 | −1.72279 | 0.00412 |
| 1569167_at | COMMD10 | COMM domain containing 10 | −1.72393 | 0.006147 |
| 205801_s_at | RASGRP3 | RAS guanyl releasing protein 3 (calcium and DAG-regulated) | −1.72845 | 0.00425 |
| 231371_at | TDRD10 | tudor domain containing 10 | −1.72977 | 0.001684 |
| 234233_s_at | KBTBD2 | kelch repeat and BTB (POZ) domain containing 2 | −1.73363 | 0.001082 |
| 215505_s_at | STRN3 | striatin, calmodulin binding protein 3 | −1.73855 | 0.000878 |
| 1556633_at | C1orf204 | chromosome 1 open reading frame 204 | −1.73967 | 0.001722 |
| 226795_at | LRCH1 | Leucine-rich repeats and calponin homology (CH) domain containing 1 | −1.74394 | 0.007592 |
| 240282_at | WDR1 | WD repeat domain 1 | −1.74449 | 0.008613 |
| 205150_s_at | KIAA0644 | KIAA0644 gene product | −1.74619 | 0.00207 |
| 227046_at | SLC39A11 | solute carrier family 39 (metal ion transporter), member 11 | −1.74656 | 0.000445 |
| 210380_s_at | CACNA1G | calcium channel, voltage-dependent, alpha 1G subunit | −1.74741 | 0.000276 |
| 205507_at | ARHGEF15 | Rho guanine nucleotide exchange factor (GEF) 15 | −1.75263 | 0.000199 |
| 207266_x_at | RBMS1 /// | RNA binding motif, single stranded interacting protein 1 /// region | −1.75318 | 0.007008 |
| | LOC648293 | containing chromosome 2 open reading frame 12; RNA binding motif, | | |
| | | single stranded interacting protein 1 | | |
| 230276_at | — | — | −1.75422 | 2.08E−05 |
| 209191_at | TUBB6 | tubulin, beta 6 | −1.75744 | 0.002728 |
| 200878_at | EPAS1 | endothelial PAS domain protein 1 | −1.75852 | 0.006394 |
| 224992_s_at | CMIP | c-Maf-inducing protein | −1.76176 | 0.00085 |
| 223075_s_at | C9orf58 | chromosome 9 open reading frame 58 | −1.76202 | 0.006981 |
| 228368_at | ARHGAP20 | Rho GTPase activating protein 20 | −1.7633 | 0.001453 |
| 227957_at | — | — | −1.7638 | 0.005881 |
| 240964_at | PTEN | Phosphatase and tensin homolog (mutated in multiple advanced | −1.76485 | 0.00821 |
| | | cancers 1) | | |
| 226368_at | CHST11 | Carbohydrate (chondroitin 4) sulfotransferase 11 | −1.76656 | 0.000193 |
| 236343_at | FXC1 | Fracture callus 1 homolog (rat) | −1.77698 | 0.005112 |
| 1557400_at | — | CDNA FLJ38935 fis, clone NT2NE2014681 | −1.77914 | 0.001242 |
| 235570_at | — | CDNA FLJ36544 fis, clone TRACH2006378 | −1.77917 | 0.005345 |
| 201147_s_at | TIMP3 | TIMP metallopeptidase inhibitor 3 (Sorsby fundus dystrophy, | −1.78031 | 0.004488 |
| | | pseudoinflammatory) | | |
| 223395_at | ABI3BP | ABI gene family, member 3 (NESH) binding protein | −1.78787 | 2.83E−05 |
| 230150_at | BCAP29 | B-cell receptor-associated protein 29 | −1.78845 | 0.000433 |
| 1553194_at | NEGR1 | neuronal growth regulator 1 | −1.78881 | 0.009605 |
| 238736_at | REV3L | REV3-like, catalytic subunit of DNA polymerase zeta (yeast) | −1.7913 | 0.000446 |
| 230300_at | — | CDNA FLJ42315 fis, clone TRACH2019661 | −1.79254 | 4.23E−05 |
| 218849_s_at | PPP1R13L | protein phosphatase 1, regulatory (inhibitor) subunit 13 like | −1.79343 | 0.000501 |
| 205398_s_at | SMAD3 | SMAD, mothers against DPP homolog 3 (Drosophila) | −1.798 | 0.004474 |
| 213309_at | PLCL2 | phospholipase C-like 2 | −1.79963 | 0.001383 |
| 219134_at | ELTD1 | EGF, latrophilin and seven transmembrane domain containing 1 | −1.80164 | 0.001158 |
| 235593_at | ZFHX1B | zinc finger homeobox 1b | −1.80513 | 0.004703 |
| 226677_at | ZNF521 | zinc finger protein 521 | −1.80553 | 0.002953 |
| 235318_at | FBN1 | fibrillin 1 | −1.81022 | 0.005849 |
| 244461_at | SPECC1 | spectrin domain with coiled-coils 1 | −1.81192 | 0.000511 |
| 213135_at | TIAM1 | T-cell lymphoma invasion and metastasis 1 | −1.8129 | 0.000978 |
| 225582_at | KIAA1754 | KIAA1754 | −1.81548 | 0.002831 |
| 228776_at | GJA7 | gap junction protein, alpha 7, 45 kDa (connexin 45) | −1.81549 | 0.008976 |
| 214821_at | SLC25A4 | Solute carrier family 25 (mitochondrial carrier; adenine nucleotide | −1.81576 | 0.000321 |
| | | translocator), member 4 | | |
| 243357_at | NEGR1 | neuronal growth regulator 1 | −1.81713 | 0.002154 |
| 220975_s_at | C1QTNF1 | C1q and tumor necrosis factor related protein 1 /// C1q and tumor | −1.81923 | 0.000804 |
| | | necrosis factor related protein 1 | | |
| 241925_x_at | — | Transcribed locus | −1.8194 | 0.001589 |
| 219371_s_at | KLF2 | Kruppel-like factor 2 (lung) | −1.82248 | 0.00258 |
| 238649_at | PITPNC1 | phosphatidylinositol transfer protein, cytoplasmic 1 | −1.82517 | 0.00559 |
| 227385_at | PPAPDC2 | phosphatidic acid phosphatase type 2 domain containing 2 | −1.82577 | 0.000129 |
| 226028_at | ROBO4 | roundabout homolog 4, magic roundabout (Drosophila) | −1.82607 | 0.00933 |
| 206481_s_at | LDB2 | LIM domain binding 2 | −1.82707 | 3.51E−08 |
| 214297_at | CSPG4 | Chondroitin sulfate proteoglycan 4 (melanoma-associated) | −1.8271 | 0.005204 |
| 1558199_at | FN1 | fibronectin 1 | −1.82754 | 0.000831 |
| 212817_at | DNAJB5 | DnaJ (Hsp40) homolog, subfamily B, member 5 | −1.82947 | 0.000334 |
| 204422_s_at | FGF2 | fibroblast growth factor 2 (basic) | −1.82971 | 0.002893 |
| 235680_at | STAT3 | Signal transducer and activator of transcription 3 (acute-phase | −1.83026 | 0.001801 |
| | | response factor) | | |
| 229241_at | LDHD | lactate dehydrogenase D | −1.83099 | 0.00541 |
| 227617_at | RP13-15M17.2 | hypothetical protein LOC199953 | −1.83222 | 0.005519 |
| 238740_at | AARSD1 | alanyl-tRNA synthetase domain containing 1 | −1.83233 | 0.000874 |
| 206702_at | TEK | TEK tyrosine kinase, endothelial (venous malformations, multiple | −1.83365 | 5.27E−05 |
| | | cutaneous and mucosal) | | |
| 226625_at | — | — | −1.83456 | 0.000465 |
| 238726_at | USP46 | Ubiquitin specific peptidase 46 | −1.83469 | 0.003215 |
| 1556361_s_at | ANKRD13C | ankyrin repeat domain 13C | −1.83782 | 0.004743 |
| 209976_s_at | CYP2E1 | cytochrome P450, family 2, subfamily E, polypeptide 1 | −1.8392 | 0.005247 |
| 220911_s_at | KIAA1305 | KIAA1305 | −1.83995 | 0.000225 |
| 238669_at | PTGS1 | prostaglandin-endoperoxide synthase 1 (prostaglandin G/H synthase | −1.84179 | 0.004343 |
| | | and cyclooxygenase) | | |
| 207554_x_at | TBXA2R | thromboxane A2 receptor | −1.84406 | 0.000142 |
| 226984_at | FGD5 | FYVE, RhoGEF and PH domain containing 5 | −1.84684 | 0.00013 |
| 203256_at | CDH3 | cadherin 3, type 1, P-cadherin (placental) | −1.84694 | 0.002369 |
| 242907_at | GBP2 | guanylate binding protein 2, interferon-inducible | −1.84705 | 0.007332 |
| 229902_at | LOC285682 | hypothetical protein LOC285682 | −1.84741 | 0.005632 |
| 205624_at | CPA3 | carboxypeptidase A3 (mast cell) | −1.84744 | 0.007451 |
| 206283_s_at | TAL1 | T-cell acute lymphocytic leukemia 1 | −1.848 | 0.007995 |
| 219218_at | BAHCC1 | BAH domain and coiled-coil containing 1 | −1.85048 | 0.0087 |
| 243146_at | ADRA1A | Adrenergic, alpha-1A-, receptor | −1.85172 | 0.002914 |
| 203065_s_at | CAV1 | caveolin 1, caveolae protein, 22 kDa | −1.85247 | 0.006107 |
| 228286_at | FLJ40869 | hypothetical protein FLJ40869 | −1.8567 | 0.001493 |
| 235131_at | RHOJ | ras homolog gene family, member J | −1.85742 | 0.003386 |
| 203151_at | MAP1A | microtubule-associated protein 1A | −1.85978 | 0.002778 |
| 226342_at | SPTBN1 | Spectrin, beta, non-erythrocytic 1 | −1.86351 | 0.002775 |
| 203323_at | CAV2 | caveolin 2 | −1.86423 | 7.71E−05 |
| 230519_at | FLJ30707 | hypothetical protein FLJ30707 | −1.86575 | 0.006666 |
| 205098_at | CCR1 | chemokine (C-C motif) receptor 1 | −1.86642 | 0.004977 |
| 225008_at | MGC34646 | Hypothetical protein MGC34646 | −1.86741 | 0.007466 |
| 229907_at | SLC18A2 | Solute carrier family 18 (vesicular monoamine), member 2 | −1.8676 | 0.008496 |
| 241068_at | — | Transcribed locus | −1.86857 | 0.002404 |
| 228580_at | HTRA3 | HtrA serine peptidase 3 | −1.86871 | 0.009982 |
| 207624_s_at | RPGR | retinitis pigmentosa GTPase regulator | −1.87096 | 0.000781 |
| 212750_at | PPP1R16B | protein phosphatase 1, regulatory (inhibitor) subunit 16B | −1.87221 | 0.002333 |
| 241401_at | C4orf12 | chromosome 4 open reading frame 12 | −1.87245 | 0.000273 |
| 228665_at | CYYR1 | cysteine/tyrosine-rich 1 | −1.8738 | 0.000604 |
| 235044_at | CYYR1 | cysteine/tyrosine-rich 1 | −1.8768 | 0.001565 |
| 203232_s_at | ATXN1 | ataxin 1 | −1.87799 | 0.003983 |
| 52255_s_at | COL5A3 | collagen, type V, alpha 3 | −1.87912 | 1.40E−06 |
| 235707_at | LOC221710 | hypothetical protein LOC221710 | −1.8809 | 0.009289 |
| 230266_at | RAB7B | RAB7B, member RAS oncogene family | −1.88206 | 0.004706 |
| 206377_at | FOXF2 | forkhead box F2 | −1.88421 | 0.006087 |
| 236277_at | PAK3 | P21 (CDKN1A)-activated kinase 3 | −1.88422 | 0.005973 |
| 227610_at | — | CDNA FLJ14337 fis, clone PLACE4000494 | −1.88426 | 0.007418 |
| 203973_s_at | CEBPD | CCAAT/enhancer binding protein (C/EBP), delta | −1.88582 | 0.005632 |
| 203748_x_at | RBMS1 /// | RNA binding motif, single stranded interacting protein 1 /// region | −1.88653 | 0.007631 |
| | LOC648293 | containing chromosome 2 open reading frame 12; RNA binding motif, | | |
| | | single stranded interacting protein 1 | | |
| 243209_at | KCNQ4 | potassium voltage-gated channel, KQT-like subfamily, member 4 | −1.88963 | 0.003246 |
| 228314_at | — | — | −1.88987 | 0.000486 |
| 200911_s_at | TACC1 | transforming, acidic coiled-coil containing protein 1 | −1.89007 | 1.59E−05 |
| 232715_at | — | CDNA FLJ11544 fis, clone HEMBA1002826 | −1.89087 | 2.16E−05 |
| 1569142_at | RFP2 | ret finger protein 2 | −1.89184 | 0.006401 |
| 239847_at | — | CDNA clone IMAGE: 6186815 | −1.89362 | 0.009279 |
| 205099_s_at | CCR1 | chemokine (C-C motif) receptor 1 | −1.8945 | 0.000229 |
| 226021_at | RDH10 | retinol dehydrogenase 10 (all-trans) | −1.89817 | 0.007074 |
| 208690_s_at | PDLIM1 | PDZ and LIM domain 1 (elfin) | −1.89851 | 0.000199 |
| 226069_at | PRICKLE1 | prickle-like 1 (Drosophila) | −1.89881 | 0.005413 |
| 222927_s_at | LMAN1L /// | lectin, mannose-binding, 1 like /// complexin 3 | −1.89906 | 0.002927 |
| | CPLX3 | | | |
| 1431_at | CYP2E1 | cytochrome P450, family 2, subfamily E, polypeptide 1 | −1.90032 | 0.006051 |
| 212822_at | HEG1 | HEG homolog 1 (zebrafish) | −1.90054 | 0.008726 |
| 213058_at | RP3-477H23.1 | KIAA1043 protein | −1.90362 | 0.005043 |
| 218611_at | IER5 | immediate early response 5 | −1.90401 | 0.000306 |
| 209897_s_at | SLIT2 | slit homolog 2 (Drosophila) | −1.90468 | 0.002883 |
| 242767_at | LMCD1 | LIM and cysteine-rich domains 1 | −1.90688 | 0.004022 |
| 219167_at | RASL12 | RAS-like, family 12 | −1.91116 | 0.00074 |
| 1553150_at | AOF1 | amine oxidase (flavin containing) domain 1 | −1.91128 | 0.000457 |
| 229405_at | KIF7 | kinesin family member 7 | −1.91146 | 0.000711 |
| 226712_at | SSR1 | Signal sequence receptor, alpha (translocon-associated protein alpha) | −1.91175 | 0.009151 |
| 211303_x_at | PSMAL | growth-inhibiting protein 26 | −1.91279 | 0.002349 |
| 231991_at | C20orf160 | chromosome 20 open reading frame 160 | −1.91428 | 0.00232 |
| 201328_at | ETS2 | v-ets erythroblastosis virus E26 oncogene homolog 2 (avian) | −1.91448 | 0.009935 |
| 212681_at | EPB41L3 | erythrocyte membrane protein band 4.1-like 3 | −1.91473 | 0.001666 |
| 204677_at | CDH5 | cadherin 5, type 2, VE-cadherin (vascular epithelium) | −1.91499 | 0.001783 |
| 227277_at | — | CDNA FLJ41088 fis, clone ASTRO2002459 | −1.91568 | 0.004671 |
| 229942_at | BNC2 | Basonuclin 2 | −1.91717 | 0.005502 |
| 201694_s_at | EGR1 | early growth response 1 | −1.91811 | 0.000237 |
| 225798_at | tcag7.981 | juxtaposed with another zinc finger gene 1 | −1.91869 | 0.002125 |
| 202086_at | MX1 | myxovirus (influenza virus) resistance 1, interferon-inducible protein | −1.91957 | 0.001115 |
| | | p78 (mouse) /// myxovirus (influenza virus) resistance 1, interferon- | | |
| | | inducible protein p78 (mouse) | | |
| 202479_s_at | TRIB2 | tribbles homolog 2 (Drosophila) | −1.922 | 0.006716 |
| 235267_at | LOC402560 | Hypothetical LOC402560 | −1.92638 | 0.004626 |
| 229427_at | — | Clone FBD8 Cri-du-chat critical region mRNA | −1.9272 | 0.001107 |
| 214081_at | PLXDC1 | plexin domain containing 1 | −1.93018 | 0.000659 |
| 204904_at | GJA4 | gap junction protein, alpha 4, 37 kDa (connexin 37) | −1.93197 | 1.81E−05 |
| 203498_at | DSCR1L1 | Down syndrome critical region gene 1-like 1 | −1.93199 | 0.000889 |
| 217504_at | ABCA6 | ATP-binding cassette, sub-family A (ABC1), member 6 | −1.93318 | 0.001286 |
| 209946_at | VEGFC | vascular endothelial growth factor C | −1.93674 | 0.001157 |
| 212230_at | PPAP2B | phosphatidic acid phosphatase type 2B | −1.93735 | 0.000176 |
| 232119_at | SYNPO2 | Synaptopodin 2 | −1.938 | 0.000612 |
| 232080_at | HECW2 | HECT, C2 and WW domain containing E3 ubiquitin protein ligase 2 | −1.93924 | 0.002116 |
| 1556555_at | LOC648768 | hypothetical protein LOC648768 | −1.9395 | 0.00404 |
| 230708_at | PRICKLE1 | Prickle-like 1 (Drosophila) | −1.94166 | 0.001345 |
| 206036_s_at | REL | v-rel reticuloendotheliosis viral oncogene homolog (avian) | −1.94335 | 0.005514 |
| 230381_at | C1orf186 | chromosome 1 open reading frame 186 | −1.94433 | 0.001178 |
| 230645_at | FRMD3 | FERM domain containing 3 | −1.94634 | 0.000233 |
| 1568876_a_at | — | CDNA clone IMAGE: 4820924 | −1.94725 | 0.003566 |
| 1559256_at | MAGI1 | membrane associated guanylate kinase, WW and PDZ domain | −1.94858 | 0.00773 |
| | | containing 1 | | |
| 237034_at | — | Full-length cDNA clone CS0DC025YP03 of Neuroblastoma Cot 25- | −1.95132 | 0.003032 |
| | | normalized of Homo sapiens (human) | | |
| 212415_at | 6-Sep | septin 6 | −1.96019 | 0.006618 |
| 225968_at | PRICKLE2 | prickle-like 2 (Drosophila) | −1.96075 | 0.007668 |
| 225557_at | AXUD1 | AXIN1 up-regulated 1 | −1.96569 | 0.00327 |
| 205632_s_at | PIP5K1B | phosphatidylinositol-4-phosphate 5-kinase, type I, beta | −1.96883 | 0.001101 |
| 235471_at | C10orf72 | Chromosome 10 open reading frame 72 | −1.96897 | 0.001102 |
| 204950_at | CARD8 | caspase recruitment domain family, member 8 | −1.96937 | 0.00011 |
| 1559062_at | — | Homo sapiens, clone IMAGE: 5730164, mRNA | −1.96992 | 9.52E−05 |
| 211490_at | ADRA1A | adrenergic, alpha-1A-, receptor | −1.97025 | 0.002728 |
| 213258_at | TFPI | tissue factor pathway inhibitor (lipoprotein-associated coagulation | −1.97026 | 1.44E−06 |
| | | inhibitor) | | |
| 213657_s_at | ZNF710 | Zinc finger protein 710 /// MRNA full length insert cDNA clone | −1.97031 | 0.004469 |
| | | EUROIMAGE 375854 | | |
| 213497_at | ABTB2 | ankyrin repeat and BTB (POZ) domain containing 2 | −1.9724 | 0.000618 |
| 205478_at | PPP1R1A | protein phosphatase 1, regulatory (inhibitor) subunit 1A | −1.97639 | 0.000842 |
| 236577_at | — | Transcribed locus | −1.98053 | 0.00956 |
| 1555233_at | RHOJ | ras homolog gene family, member J | −1.98069 | 0.00684 |
| 235719_at | KLKB1 | Kallikrein B, plasma (Fletcher factor) 1 | −1.98072 | 0.007653 |
| 206263_at | FMO4 | flavin containing monooxygenase 4 | −1.98096 | 0.002078 |
| 203939_at | NT5E | 5′-nucleotidase, ecto (CD73) | −1.98406 | 0.004563 |
| 232027_at | SYNE1 | Spectrin repeat containing, nuclear envelope 1 | −1.98461 | 0.005621 |
| 229073_at | LOC145786 | Hypothetical protein LOC145786 | −1.98504 | 0.008669 |
| 226096_at | FNDC5 | fibronectin type III domain containing 5 | −1.98913 | 0.005844 |
| 204468_s_at | TIE1 | tyrosine kinase with immunoglobulin-like and EGF-like domains 1 | −1.99184 | 2.36E−05 |
| 229893_at | FRMD3 | FERM domain containing 3 | −1.99213 | 0.00073 |
| 224724_at | SULF2 | sulfatase 2 | −1.99365 | 0.000252 |
| 231511_at | FRAS1 | Fraser syndrome 1 | −1.99449 | 0.0069 |
| 1558820_a_at | C18orf34 | chromosome 18 open reading frame 34 | −1.99699 | 0.009496 |
| 235489_at | RHOJ | ras homolog gene family, member J | −1.99855 | 0.004117 |
| 209685_s_at | PRKCB1 | protein kinase C, beta 1 | −2.0045 | 0.000673 |
| 225940_at | EIF4E3 | eukaryotic translation initiation factor 4E member 3 | −2.00489 | 0.005496 |
| 232687_at | — | CDNA FLJ33091 fis, clone TRACH2000660 | −2.00496 | 0.000135 |
| 221039_s_at | DDEF1 | development and differentiation enhancing factor 1 | −2.00593 | 0.007202 |
| 226942_at | PHF20L1 | PHD finger protein 20-like 1 | −2.00812 | 0.000361 |
| 208893_s_at | DUSP6 | dual specificity phosphatase 6 | −2.00889 | 3.75E−05 |
| 214717_at | DKFZp434H1419 | hypothetical protein DKFZp434H1419 | −2.01126 | 0.000622 |
| 1560359_at | PELO | Pelota homolog (Drosophila) | −2.0132 | 0.003613 |
| 225978_at | FAM80B | family with sequence similarity 80, member B | −2.01333 | 2.88E−05 |
| 239123_at | — | Colorectal cancer-related mRNA sequence | −2.01466 | 0.007866 |
| 204271_s_at | EDNRB | endothelin receptor type B | −2.01601 | 0.000769 |
| 203066_at | GALNAC4S-6ST | B cell RAG associated protein | −2.01624 | 0.000877 |
| 231478_at | — | Transcribed locus, strongly similar to NP_000914.1 phosphodiesterase | −2.01664 | 0.001009 |
| | | 4C, cAMP-specific (phosphodiesterase E1 dunce homolog, | | |
| | | Drosophila); Phosphodiesterase-4C, cAMP-specific (dunce | | |
| | | (Drosophila)-homolog; phosphodiesterase 4C, cAMP-specific (dunce | | |
| | | (Drosophila)-homolog phosphodiesterase E1) [Homo sapiens] | | |
| 203705_s_at | FZD7 | frizzled homolog 7 (Drosophila) | −2.01832 | 0.000756 |
| 201462_at | SCRN1 | secernin 1 | −2.01833 | 0.004171 |
| 242809_at | IL1RL1 | Interleukin 1 receptor-like 1 | −2.01972 | 0.003473 |
| 213392_at | MGC35048 | hypothetical protein MGC35048 | −2.01976 | 0.000235 |
| 214622_at | CYP21A2 | cytochrome P450, family 21, subfamily A, polypeptide 2 | −2.01989 | 0.003397 |
| 236583_at | ABP1 | Amiloride binding protein 1 (amine oxidase (copper-containing)) | −2.0208 | 0.003752 |
| 205128_x_at | PTGS1 | prostaglandin-endoperoxide synthase 1 (prostaglandin G/H synthase | −2.02359 | 0.008498 |
| | | and cyclooxygenase) | | |
| 40560_at | TBX2 | T-box 2 | −2.0243 | 0.000601 |
| 243690_at | TRIOBP | TRIO and F-actin binding protein | −2.02734 | 2.22E−06 |
| 1555929_s_at | — | Transcribed locus | −2.02816 | 0.007389 |
| 242170_at | ZNF154 | Zinc finger protein 154 (pHZ-92) | −2.02851 | 0.002284 |
| 216485_s_at | TPSAB1 | tryptase alpha/beta 1 | −2.0296 | 0.000527 |
| 210299_s_at | FHL1 | four and a half LIM domains 1 | −2.02961 | 0.003873 |
| 241381_at | CXorf36 | chromosome X open reading frame 36 | −2.03073 | 0.000195 |
| 240603_s_at | EXOD1 | exonuclease domain containing 1 | −2.03271 | 0.003565 |
| 37028_at | PPP1R15A | protein phosphatase 1, regulatory (inhibitor) subunit 15A | −2.03279 | 0.007306 |
| 242708_at | PEX1 | Peroxisome biogenesis factor 1 | −2.03368 | 0.001527 |
| 240024_at | SEC14L2 | SEC14-like 2 (S. cerevisiae) | −2.03521 | 0.009161 |
| 232746_at | CMKOR1 | Chemokine orphan receptor 1 | −2.03729 | 0.001009 |
| 208438_s_at | FGR | Gardner-Rasheed feline sarcoma viral (v-fgr) oncogene homolog | −2.03825 | 0.009721 |
| 202878_s_at | CD93 | CD93 molecule | −2.03986 | 0.000107 |
| 218880_at | FOSL2 | FOS-like antigen 2 | −2.04194 | 0.002046 |
| 228311_at | BCL6B | B-cell CLL/lymphoma 6, member B (zinc finger protein) | −2.04316 | 0.004588 |
| 239503_at | — | CDNA clone IMAGE: 5301910 | −2.04346 | 0.007943 |
| 218330_s_at | NAV2 | neuron navigator 2 | −2.04428 | 0.000699 |
| 229803_s_at | — | Transcribed locus | −2.04581 | 0.000133 |
| 234996_at | — | Transcribed locus | −2.04771 | 0.003079 |
| 218807_at | VAV3 | vav 3 oncogene | −2.04861 | 0.002102 |
| 224929_at | LOC340061 | hypothetical protein LOC340061 | −2.04951 | 0.007988 |
| 226071_at | ADAMTSL4 | ADAMTS-like 4 | −2.0509 | 0.002446 |
| 230773_at | — | Transcribed locus | −2.05139 | 0.009677 |
| 209047_at | AQP1 | aquaporin 1 (Colton blood group) | −2.05478 | 1.05E−06 |
| 228937_at | FLJ38725 | hypothetical protein FLJ38725 | −2.05598 | 0.000883 |
| 204794_at | DUSP2 | dual specificity phosphatase 2 | −2.0566 | 0.000361 |
| 236383_at | — | Transcribed locus | −2.05821 | 0.000156 |
| 203921_at | CHST2 | carbohydrate (N-acetylglucosamine-6-O) sulfotransferase 2 | −2.06113 | 0.00058 |
| 213075_at | OLFML2A | olfactomedin-like 2A | −2.06235 | 0.000991 |
| 230052_s_at | TA-NFKBH | T-cell activation NFKB-like protein | −2.06437 | 0.003047 |
| 224337_s_at | FZD4 | frizzled homolog 4 (Drosophila) /// frizzled homolog 4 (Drosophila) | −2.06527 | 0.001901 |
| 227611_at | TARSL2 | threonyl-tRNA synthetase-like 2 | −2.06665 | 0.001075 |
| 202241_at | TRIB1 | tribbles homolog 1 (Drosophila) | −2.07301 | 0.006084 |
| 205994_at | ELK4 | ELK4, ETS-domain protein (SRF accessory protein 1) | −2.07318 | 0.004532 |
| 230800_at | ADCY4 | adenylate cyclase 4 | −2.0742 | 3.41E−05 |
| 212298_at | NRP1 | neuropilin 1 | −2.08047 | 0.001938 |
| 226372_at | CHST11 | Carbohydrate (chondroitin 4) sulfotransferase 11 | −2.08392 | 0.000413 |
| 213744_at | ATRNL1 | attractin-like 1 | −2.08476 | 0.002684 |
| 225939_at | EIF4E3 | eukaryotic translation initiation factor 4E member 3 | −2.08552 | 0.000539 |
| 204182_s_at | ZBTB43 | zinc finger and BTB domain containing 43 | −2.08878 | 0.000271 |
| 215318_at | CG012 | hypothetical gene CG012 | −2.08952 | 0.001058 |
| 229380_at | — | Transcribed locus, weakly similar to NP_060312.1 hypothetical protein | −2.0919 | 9.04E−05 |
| | | FLJ20489 [Homo sapiens] | | |
| 229011_at | — | Transcribed locus | −2.09219 | 0.000251 |
| 208791_at | CLU | clusterin | −2.09476 | 0.004765 |
| 210824_at | — | — | −2.09481 | 0.004728 |
| 205330_at | MN1 | meningioma (disrupted in balanced translocation) 1 | −2.09518 | 0.005078 |
| 210471_s_at | KCNAB1 | potassium voltage-gated channel, shaker-related subfamily, beta | −2.09854 | 0.003167 |
| | | member 1 | | |
| 209868_s_at | RBMS1 /// | RNA binding motif, single stranded interacting protein 1 /// region | −2.09886 | 0.006349 |
| | LOC648293 | containing chromosome 2 open reading frame 12; RNA binding motif, | | |
| | | single stranded interacting protein 1 | | |
| 40687_at | GJA4 | gap junction protein, alpha 4, 37 kDa (connexin 37) | −2.09956 | 0.004302 |
| 227684_at | DNMT1 | DNA (cytosine-5-)-methyltransferase 1 | −2.1002 | 0.000118 |
| 1555950_a_at | CD55 | CD55 molecule, decay accelerating factor for complement (Cromer | −2.10554 | 7.51E−05 |
| | | blood group) | | |
| 201289_at | CYR61 | cysteine-rich, angiogenic inducer, 61 | −2.10663 | 0.001464 |
| 204518_s_at | PPIC | peptidylprolyl isomerase C (cyclophilin C) | −2.10695 | 0.000621 |
| 203037_s_at | MTSS1 | metastasis suppressor 1 | −2.10991 | 0.005191 |
| 239089_at | — | Transcribed locus | −2.11058 | 0.003484 |
| 209324_s_at | RGS16 | regulator of G-protein signalling 16 | −2.11173 | 0.009126 |
| 221840_at | PTPRE | protein tyrosine phosphatase, receptor type, E | −2.11315 | 0.00349 |
| 227061_at | — | CDNA FLJ44429 fis, clone UTERU2015653 | −2.1142 | 0.001127 |
| 1566968_at | SPRY4 | Sprouty homolog 4 (Drosophila) | −2.11837 | 0.009358 |
| 206837_at | CART1 | cartilage paired-class homeoprotein 1 | −2.11885 | 0.001543 |
| 236586_at | CLNS1A | Chloride channel, nucleotide-sensitive, 1A | −2.11999 | 0.002569 |
| 242123_at | PAQR7 | progestin and adipoQ receptor family member VII | −2.12502 | 0.007007 |
| 202566_s_at | SVIL | supervillin | −2.12517 | 0.002679 |
| 229802_at | — | CDNA FLJ14388 fis, clone HEMBA1002716 | −2.12592 | 0.002179 |
| 218062_x_at | CDC42EP4 | CDC42 effector protein (Rho GTPase binding) 4 | −2.12849 | 0.009663 |
| 225511_at | GPRC5B | G protein-coupled receptor, family C, group 5, member B | −2.12878 | 8.92E−05 |
| 207876_s_at | FLNC | filamin C, gamma (actin binding protein 280) | −2.12916 | 0.00789 |
| 211776_s_at | EPB41L3 | erythrocyte membrane protein band 4.1-like 3 /// erythrocyte membrane | −2.1311 | 0.006831 |
| | | protein band 4.1-like 3 | | |
| 230962_at | — | Clones 24632 and 24634 mRNA sequence | −2.13874 | 0.007406 |
| 210664_s_at | TFPI | tissue factor pathway inhibitor (lipoprotein-associated coagulation | −2.13915 | 0.002607 |
| | | inhibitor) | | |
| 214954_at | SUSD5 | sushi domain containing 5 | −2.14076 | 0.001102 |
| 217023_x_at | TPSAB1 | tryptase alpha/beta 1 | −2.14394 | 0.000674 |
| 214632_at | NRP2 | neuropilin 2 | −2.14589 | 0.000502 |
| 239092_at | ITGA8 | integrin, alpha 8 | −2.14615 | 0.00828 |
| 1556110_at | — | CDNA FLJ39819 fis, clone SPLEN2010534 | −2.14652 | 0.002602 |
| 229943_at | RFP2 | ret finger protein 2 | −2.14881 | 0.003334 |
| 235831_at | — | Transcribed locus | −2.15332 | 0.008027 |
| 241302_at | C7 | Complement component 7 | −2.15471 | 0.002396 |
| 203144_s_at | KIAA0040 | KIAA0040 | −2.1596 | 0.000121 |
| 230286_at | — | Transcribed locus | −2.15961 | 3.88E−05 |
| 215813_s_at | PTGS1 | prostaglandin-endoperoxide synthase 1 (prostaglandin G/H synthase | −2.16065 | 0.005541 |
| | | and cyclooxygenase) | | |
| 230161_at | CD99 | CD99 molecule | −2.16098 | 0.001758 |
| 1555938_x_at | VIM | vimentin | −2.16151 | 0.007369 |
| 209789_at | CORO2B | coronin, actin binding protein, 2B | −2.16225 | 0.00015 |
| 229723_at | TAGAP | T-cell activation GTPase activating protein | −2.16311 | 0.003628 |
| 207277_at | CD209 | CD209 molecule | −2.16506 | 0.004652 |
| 230061_at | TM4SF18 | Transmembrane 4 L six family member 18 | −2.16524 | 0.000178 |
| 210786_s_at | FLI1 | Friend leukemia virus integration 1 | −2.16853 | 0.002959 |
| 238169_at | — | Transcribed locus | −2.16882 | 0.002861 |
| 213994_s_at | SPON1 | spondin 1, extracellular matrix protein | −2.16894 | 0.002634 |
| 212558_at | SPRY1 | sprouty homolog 1, antagonist of FGF signaling (Drosophila) | −2.17174 | 6.19E−08 |
| 205111_s_at | PLCE1 | phospholipase C, epsilon 1 | −2.17228 | 0.000141 |
| 201926_s_at | CD55 | CD55 molecule, decay accelerating factor for complement (Cromer | −2.17406 | 0.000226 |
| | | blood group) | | |
| 216474_x_at | TPSAB1 | tryptase alpha/beta 1 | −2.17535 | 0.000746 |
| 241756_at | SMARCA2 | SWI/SNF related, matrix associated, actin dependent regulator of | −2.17581 | 0.00322 |
| | | chromatin, subfamily a, member 2 | | |
| 238835_at | — | Transcribed locus | −2.17603 | 0.00307 |
| 1553142_at | FLJ38725 | hypothetical protein FLJ38725 | −2.17616 | 0.002679 |
| 209199_s_at | MEF2C | MADS box transcription enhancer factor 2, polypeptide C (myocyte | −2.17791 | 0.002135 |
| | | enhancer factor 2C) | | |
| 233076_at | C10orf39 | chromosome 10 open reading frame 39 | −2.17824 | 0.006715 |
| 243254_at | HIVEP2 | Human immunodeficiency virus type I enhancer binding protein 2 | −2.18244 | 0.001352 |
| 219761_at | CLEC1A | C-type lectin domain family 1, member A | −2.1826 | 0.002095 |
| 238469_at | C6orf155 | Chromosome 6 open reading frame 155 | −2.18271 | 0.00052 |
| 1554966_a_at | DOC1 | downregulated in ovarian cancer 1 | −2.18527 | 0.001684 |
| 336_at | TBXA2R | thromboxane A2 receptor | −2.18769 | 0.00013 |
| 240823_at | — | Homo sapiens, clone IMAGE: 5730164, mRNA | −2.19237 | 1.85E−05 |
| 227647_at | KCNE3 | potassium voltage-gated channel, Isk-related family, member 3 | −2.19245 | 0.002724 |
| 213317_at | CLIC5 /// | chloride intracellular channel 5 /// similar to chloride intracellular | −2.19494 | 0.001923 |
| | LOC653816 | channel 5 | | |
| 227467_at | RDH10 | retinol dehydrogenase 10 (all-trans) | −2.19659 | 0.002935 |
| 207134_x_at | TPSAB1 | tryptase alpha/beta 1 | −2.19949 | 0.000528 |
| 209842_at | SOX10 | SRY (sex determining region Y)-box 10 | −2.20027 | 0.002953 |
| 209283_at | CRYAB | crystallin, alpha B | −2.20146 | 0.000427 |
| 215382_x_at | TPSAB1 | tryptase alpha/beta 1 | −2.20197 | 0.000853 |
| 238546_at | SLC8A1 | solute carrier family 8 (sodium/calcium exchanger), member 1 | −2.202 | 0.008528 |
| 217437_s_at | TACC1 | transforming, acidic coiled-coil containing protein 1 | −2.20528 | 0.000239 |
| 202242_at | TSPAN7 | tetraspanin 7 | −2.20668 | 0.000192 |
| 1552718_at | HARS2 | histidyl-tRNA synthetase 2 | −2.20789 | 0.009093 |
| 31845_at | ELF4 | E74-like factor 4 (ets domain transcription factor) | −2.20798 | 0.002084 |
| 210084_x_at | TPSAB1 | tryptase alpha/beta 1 | −2.20941 | 0.000349 |
| 225262_at | FOSL2 | FOS-like antigen 2 | −2.21028 | 0.006593 |
| 229172_at | HSPA12B | heat shock 70 kD protein 12B | −2.21287 | 0.00239 |
| 219935_at | ADAMTS5 | ADAM metallopeptidase with thrombospondin type 1 motif, 5 | −2.21402 | 0.002295 |
| | | (aggrecanase-2) | | |
| 227032_at | PLXNA2 | plexin A2 | −2.21751 | 3.52E−05 |
| 227376_at | — | Transcribed locus | −2.2219 | 0.000721 |
| 227345_at | TNFRSF10D | tumor necrosis factor receptor superfamily, member 10d, decoy with | −2.22206 | 0.000297 |
| | | truncated death domain | | |
| 1565627_a_at | LRRK1 | Leucine-rich repeat kinase 1 | −2.22741 | 0.00011 |
| 225999_at | FAM80B | family with sequence similarity 80, member B | −2.22909 | 2.81E−08 |
| 222890_at | HSPC065 | HSPC065 protein | −2.23095 | 0.006554 |
| 227297_at | ITGA9 | integrin, alpha 9 | −2.23158 | 0.00015 |
| 210692_s_at | SLC43A3 | solute carrier family 43, member 3 | −2.23452 | 0.006496 |
| 37590_g_at | ZNF710 | Zinc finger protein 710 /// MRNA full length insert cDNA clone | −2.23515 | 0.002664 |
| | | EUROIMAGE 375854 | | |
| 232102_at | METTL6 | Methyltransferase like 6 | −2.23561 | 0.001128 |
| 207741_x_at | TPSAB1 /// | tryptase alpha/beta 1 /// tryptase beta 2 /// similar to Alpha-tryptase | −2.23888 | 8.24E−05 |
| | TPSB2 /// | precursor (Tryptase 1) | | |
| | LOC652751 | | | |
| 217998_at | PHLDA1 | pleckstrin homology-like domain, family A, member 1 | −2.23893 | 0.002782 |
| 225344_at | NCOA7 | nuclear receptor coactivator 7 | −2.23932 | 0.000596 |
| 235368_at | ADAMTS5 | ADAM metallopeptidase with thrombospondin type 1 motif, 5 | −2.24398 | 0.003439 |
| | | (aggrecanase-2) | | |
| 209834_at | CHST3 | carbohydrate (chondroitin 6) sulfotransferase 3 | −2.24463 | 0.000964 |
| 229838_at | NUCB2 | nucleobindin 2 | −2.24517 | 0.008474 |
| 224964_s_at | GNG2 | guanine nucleotide binding protein (G protein), gamma 2 | −2.25172 | 0.000739 |
| 210078_s_at | KCNAB1 | potassium voltage-gated channel, shaker-related subfamily, beta | −2.25332 | 0.002677 |
| | | member 1 | | |
| 225435_at | SSR1 | Signal sequence receptor, alpha (translocon-associated protein alpha) | −2.25576 | 0.004311 |
| 205683_x_at | TPSAB1 | tryptase alpha/beta 1 | −2.25688 | 7.85E−05 |
| 231130_at | FKBP7 | FK506 binding protein 7 | −2.25807 | 1.88E−05 |
| 217525_at | OLFML1 | olfactomedin-like 1 | −2.25895 | 0.001133 |
| 232686_at | SIGLECP3 | sialic acid binding Ig-like lectin, pseudogene 3 | −2.26433 | 7.97E−05 |
| 239952_at | — | — | −2.26621 | 0.000175 |
| 231055_at | — | Transcribed locus | −2.26715 | 0.000885 |
| 206026_s_at | TNFAIP6 | tumor necrosis factor, alpha-induced protein 6 | −2.2708 | 0.001914 |
| 209956_s_at | CAMK2B | calcium/calmodulin-dependent protein kinase (CaM kinase) II beta | −2.27196 | 0.002261 |
| 244308_at | — | — | −2.27542 | 0.004925 |
| 229537_at | LMO4 | LIM domain only 4 | −2.27627 | 0.0004 |
| 235673_at | — | CDNA FLJ42198 fis, clone THYMU2034338 | −2.27806 | 0.000184 |
| 230250_at | PTPRB | Protein tyrosine phosphatase, receptor type, B | −2.2857 | 1.37E−06 |
| 226304_at | HSPB6 | heat shock protein, alpha-crystallin-related, B6 | −2.28627 | 0.001902 |
| 235183_at | — | CDNA clone IMAGE: 5312689 | −2.28824 | 0.004847 |
| 230144_at | — | — | −2.29157 | 0.002247 |
| 206331_at | CALCRL | calcitonin receptor-like | −2.29279 | 0.000123 |
| 242137_at | — | CDNA FLJ36544 fis, clone TRACH2006378 | −2.29569 | 0.005191 |
| 204731_at | TGFBR3 | transforming growth factor, beta receptor III (betaglycan, 300 kDa) | −2.29894 | 3.74E−05 |
| 230067_at | FLJ30707 | Hypothetical protein FLJ30707 | −2.29956 | 0.00956 |
| 217897_at | FXYD6 | FXYD domain containing ion transport regulator 6 | −2.30209 | 0.000242 |
| 238823_at | FMNL3 | formin-like 3 | −2.30312 | 0.009054 |
| 204421_s_at | FGF2 | fibroblast growth factor 2 (basic) | −2.30374 | 8.72E−06 |
| 205696_s_at | GFRA1 | GDNF family receptor alpha 1 | −2.30376 | 0.006663 |
| 209543_s_at | CD34 | CD34 molecule | −2.30996 | 7.85E−05 |
| 204364_s_at | REEP1 | receptor accessory protein 1 | −2.31114 | 0.001187 |
| 243023_at | — | Transcribed locus | −2.31305 | 0.001478 |
| 222885_at | EMCN | endomucin | −2.31391 | 0.003371 |
| 235292_at | FLJ32255 /// | hypothetical protein LOC643977 /// hypothetical protein LOC648075 | −2.3151 | 0.000157 |
| | LOC648075 | | | |
| 236262_at | MMRN2 | multimerin 2 | −2.31679 | 5.41E−06 |
| 223843_at | SCARA3 | scavenger receptor class A, member 3 | −2.31781 | 0.007573 |
| 1552726_at | ADAMTS17 | ADAM metallopeptidase with thrombospondin type 1 motif, 17 | −2.33328 | 8.72E−05 |
| 230170_at | OSM | oncostatin M | −2.33521 | 0.002477 |
| 235173_at | LOC401093 | hypothetical LOC401093 | −2.33707 | 2.54E−06 |
| 225784_s_at | KIAA1166 | KIAA1166 | −2.33728 | 0.008814 |
| 213925_at | C1orf95 | Chromosome 1 open reading frame 95 | −2.33807 | 0.00351 |
| 207808_s_at | PROS1 | protein S (alpha) | −2.34049 | 0.003537 |
| 215617_at | — | CDNA FLJ11754 fis, clone HEMBA1005588 | −2.34333 | 0.002133 |
| 38037_at | HBEGF | heparin-binding EGF-like growth factor | −2.3473 | 0.008543 |
| 1562012_at | — | — | −2.34997 | 0.003891 |
| 211864_s_at | FER1L3 | fer-1-like 3, myoferlin (C. elegans) | −2.35168 | 0.001888 |
| 230606_at | GJC1 | Gap junction protein, chi 1, 31.9 kDa (connexin 31.9) | −2.35575 | 0.001688 |
| 210762_s_at | DLC1 | deleted in liver cancer 1 | −2.35728 | 0.000524 |
| 226638_at | ARHGAP23 | Rho GTPase activating protein 23 | −2.36014 | 7.10E−06 |
| 212724_at | RND3 | Rho family GTPase 3 | −2.36164 | 0.004649 |
| 237058_x_at | SLC6A13 | solute carrier family 6 (neurotransmitter transporter, GABA), member | −2.36427 | 0.002485 |
| | | 13 | | |
| 209437_s_at | SPON1 | spondin 1, extracellular matrix protein | −2.3647 | 4.75E−05 |
| 227874_at | — | MRNA; cDNA DKFZp586N0121 (from clone DKFZp586N0121) | −2.3666 | 0.009573 |
| 201925_s_at | CD55 | CD55 molecule, decay accelerating factor for complement (Cromer | −2.37119 | 6.35E−06 |
| | | blood group) | | |
| 224219_s_at | TRPC4 | transient receptor potential cation channel, subfamily C, member 4 | −2.37466 | 0.002531 |
| 210815_s_at | CALCRL | calcitonin receptor-like | −2.37561 | 0.007243 |
| 1556328_at | PCDH10 | Protocadherin 10 | −2.38022 | 0.00599 |
| 209200_at | MEF2C | MADS box transcription enhancer factor 2, polypeptide C (myocyte | −2.38111 | 4.97E−05 |
| | | enhancer factor 2C) | | |
| 218660_at | DYSF | dysferlin, limb girdle muscular dystrophy 2B (autosomal recessive) | −2.38736 | 2.27E−06 |
| 207968_s_at | MEF2C | MADS box transcription enhancer factor 2, polypeptide C (myocyte | −2.38896 | 0.002435 |
| | | enhancer factor 2C) | | |
| 229696_at | — | Transcribed locus | −2.39016 | 7.46E−05 |
| 219091_s_at | MMRN2 | multimerin 2 | −2.39025 | 8.01E−05 |
| 205011_at | LOH11CR2A | loss of heterozygosity, 11, chromosomal region 2, gene A | −2.39222 | 7.44E−07 |
| 204472_at | GEM | GTP binding protein overexpressed in skeletal muscle | −2.39622 | 0.00597 |
| 229430_at | C8orf46 | chromosome 8 open reading frame 46 | −2.39819 | 0.003069 |
| 236772_s_at | — | Transcribed locus | −2.40112 | 0.001579 |
| 227722_at | RPS23 | ribosomal protein S23 | −2.40134 | 0.005661 |
| 206701_x_at | EDNRB | endothelin receptor type B | −2.40207 | 0.000118 |
| 239919_at | LOC255480 | hypothetical protein LOC255480 | −2.40305 | 0.004933 |
| 202877_s_at | CD93 | CD93 molecule /// CD93 molecule | −2.40377 | 0.000111 |
| 202627_s_at | SERPINE1 | serpin peptidase inhibitor, clade E (nexin, plasminogen activator | −2.40523 | 0.004993 |
| | | inhibitor type 1), member 1 | | |
| 1558858_at | — | CDNA FLJ35222 fis, clone PROST2000835 | −2.40742 | 0.004322 |
| 203934_at | KDR | kinase insert domain receptor (a type III receptor tyrosine kinase) | −2.40851 | 5.20E−05 |
| 236936_at | — | — | −2.41045 | 0.000421 |
| 226822_at | STOX2 | storkhead box 2 | −2.41086 | 3.62E−05 |
| 221796_at | NTRK2 | neurotrophic tyrosine kinase, receptor, type 2 | −2.41618 | 0.000652 |
| 204368_at | SLCO2A1 | solute carrier organic anion transporter family, member 2A1 | −2.41653 | 0.000446 |
| 244335_at | — | Transcribed locus | −2.41886 | 0.000265 |
| 214767_s_at | HSPB6 | heat shock protein, alpha-crystallin-related, B6 | −2.41913 | 0.0024 |
| 205100_at | GFPT2 | glutamine-fructose-6-phosphate transaminase 2 | −2.43074 | 0.00229 |
| 213560_at | GADD45B | Growth arrest and DNA-damage-inducible, beta | −2.43768 | 0.000873 |
| 205088_at | CXorf6 | chromosome X open reading frame 6 | −2.43847 | 0.001652 |
| 213993_at | SPON1 | spondin 1, extracellular matrix protein | −2.44209 | 0.00053 |
| 217177_s_at | — | CDNA FLJ13658 fis, clone PLACE1011567 | −2.44989 | 0.00034 |
| 227618_at | FLJ44635 | TPT1-like protein | −2.45198 | 0.0029 |
| 235561_at | — | Transcribed locus | −2.45367 | 0.000309 |
| 228176_at | EDG3 | endothelial differentiation, sphingolipid G-protein-coupled receptor, 3 | −2.4554 | 1.45E−05 |
| 201425_at | ALDH2 | aldehyde dehydrogenase 2 family (mitochondrial) | −2.45595 | 0.000152 |
| 211737_x_at | PTN | pleiotrophin (heparin binding growth factor 8, neurite growth-promoting | −2.46441 | 0.000156 |
| | | factor 1) /// pleiotrophin (heparin binding growth factor 8, neurite | | |
| | | growth-promoting factor 1) | | |
| 231259_s_at | — | Transcribed locus | −2.46598 | 0.003845 |
| 241992_at | FLJ11259 | Hypothetical protein FLJ11259 | −2.4672 | 0.003908 |
| 230908_at | — | Transcribed locus | −2.46789 | 2.89E−05 |
| 215059_at | — | MRNA; cDNA DKFZp564G112 (from clone DKFZp564G112) | −2.46841 | 0.000467 |
| 218980_at | FHOD3 | formin homology 2 domain containing 3 | −2.4703 | 0.005147 |
| 225842_at | PHLDA1 | pleckstrin homology-like domain, family A, member 1 | −2.47548 | 0.00033 |
| 222033_s_at | FLT1 | Fms-related tyrosine kinase 1 (vascular endothelial growth | −2.48337 | 0.00025 |
| | | factor/vascular permeability factor receptor) | | |
| 1552400_a_at | C15orf27 | chromosome 15 open reading frame 27 | −2.48507 | 0.009571 |
| 218678_at | NES | nestin | −2.4901 | 0.001932 |
| 221031_s_at | APOLD1 | apolipoprotein L domain containing 1 /// apolipoprotein L domain | −2.49233 | 0.009129 |
| | | containing 1 | | |
| 203438_at | STC2 | stanniocalcin 2 | −2.49333 | 0.001292 |
| 227554_at | LOC402560 | Hypothetical LOC402560 | −2.49503 | 0.00059 |
| 213479_at | NPTX2 | neuronal pentraxin II | −2.497 | 0.00062 |
| 219563_at | C14orf139 | chromosome 14 open reading frame 139 | −2.49719 | 1.37E−06 |
| 232122_s_at | VEPH1 | ventricular zone expressed PH domain homolog 1 (zebrafish) | −2.50584 | 0.008468 |
| 222599_s_at | NAV2 | neuron navigator 2 | −2.50655 | 0.000873 |
| 238226_at | FAM70B | Family with sequence similarity 70, member B | −2.50798 | 7.48E−07 |
| 209975_at | CYP2E1 | cytochrome P450, family 2, subfamily E, polypeptide 1 | −2.51938 | 0.008635 |
| 230109_at | PDE7B | phosphodiesterase 7B | −2.52417 | 0.000141 |
| 213658_at | ZNF710 | Zinc finger protein 710 | −2.52551 | 0.001254 |
| 1559725_at | — | MRNA; cDNA DKFZp667J0623 (from clone DKFZp667J0623) | −2.53192 | 0.003244 |
| 218523_at | LHPP | phospholysine phosphohistidine inorganic pyrophosphate phosphatase | −2.53237 | 1.47E−05 |
| 235199_at | — | Transcribed locus | −2.53433 | 0.000892 |
| 226856_at | MUSTN1 | musculoskeletal, embryonic nuclear protein 1 | −2.53984 | 0.003081 |
| 224822_at | DLC1 | deleted in liver cancer 1 | −2.54028 | 4.99E−06 |
| 207957_s_at | PRKCB1 | Protein kinase C, beta 1 | −2.54564 | 0.000454 |
| 207016_s_at | ALDH1A2 | aldehyde dehydrogenase 1 family, member A2 | −2.54747 | 0.006773 |
| 226533_at | — | — | −2.5506 | 0.00132 |
| 216918_s_at | DST | dystonin | −2.56202 | 0.001286 |
| 226492_at | SEMA6D | sema domain, transmembrane domain (TM), and cytoplasmic domain, | −2.56693 | 0.003666 |
| | | (semaphorin) 6D | | |
| 1553243_at | ITIH5 | inter-alpha (globulin) inhibitor H5 | −2.57355 | 0.000713 |
| 228375_at | IGSF11 | immunoglobulin superfamily, member 11 | −2.57391 | 0.001617 |
| 205935_at | FOXF1 | forkhead box F1 | −2.57884 | 6.07E−05 |
| 1560724_at | — | CDNA FLJ33564 fis, clone BRAMY2010135 | −2.58459 | 0.003068 |
| 226705_at | FGFR1 | fibroblast growth factor receptor 1 (fms-related tyrosine kinase 2, | −2.58528 | 0.000909 |
| | | Pfeiffer syndrome) | | |
| 209436_at | SPON1 | spondin 1, extracellular matrix protein | −2.58997 | 0.000139 |
| 213386_at | RNF20 | Ring finger protein 20 | −2.59077 | 0.003419 |
| 1556111_s_at | — | CDNA FLJ39819 fis, clone SPLEN2010534 | −2.59147 | 0.005269 |
| 204575_s_at | MMP19 /// | matrix metallopeptidase 19 /// similar to Matrix metalloproteinase-19 | −2.59789 | 0.002604 |
| | LOC652543 | precursor (MMP-19) (Matrix metalloproteinase RASI) (MMP-18) | | |
| 233947_s_at | LOC255480 | hypothetical protein LOC255480 | −2.60043 | 0.006476 |
| 214831_at | ELK4 | ELK4, ETS-domain protein (SRF accessory protein 1) | −2.60185 | 2.00E−06 |
| 205870_at | BDKRB2 | bradykinin receptor B2 | −2.60591 | 0.000451 |
| 235545_at | — | — | −2.60761 | 0.000733 |
| 206684_s_at | ATF7 | activating transcription factor 7 | −2.61277 | 0.0009 |
| 205114_s_at | CCL3 /// CCL3L1 | chemokine (C-C motif) ligand 3 /// chemokine (C-C motif) ligand 3-like 1 | −2.62497 | 0.00757 |
| | /// CCL3L3 /// | /// chemokine (C-C motif) ligand 3-like 3 /// similar to Small inducible | | |
| | LOC643930 | cytokine A3-like 1 precursor (Tonsillar lymphocyte LD78 beta protein) | | |
| | | (LD78-beta(1-70)) (G0/G1 switch regulatory protein 19-2) (G0S19-2 | | |
| | | protein) (PAT 464.2) | | |
| 213652_at | PCSK5 | Proprotein convertase subtilisin/kexin type 5 | −2.63083 | 4.40E−06 |
| 227058_at | FLJ14834 | hypothetical protein FLJ14834 | −2.63328 | 0.002721 |
| 223595_at | TMEM133 | transmembrane protein 133 | −2.64126 | 0.000656 |
| 228010_at | PPP2R2C | protein phosphatase 2 (formerly 2A), regulatory subunit B (PR 52), | −2.64539 | 1.70E−06 |
| | | gamma isoform | | |
| 202014_at | PPP1R15A | protein phosphatase 1, regulatory (inhibitor) subunit 15A | −2.64581 | 0.001523 |
| 221489_s_at | SPRY4 /// | sprouty homolog 4 (Drosophila) /// similar to sprouty homolog 4 | −2.65098 | 0.000109 |
| | LOC653170 | (Drosophila) | | |
| 217996_at | PHLDA1 | pleckstrin homology-like domain, family A, member 1 | −2.65728 | 4.80E−05 |
| 230025_at | GJC1 | gap junction protein, chi 1, 31.9 kDa (connexin 31.9) | −2.66303 | 0.00207 |
| 231257_at | TCERG1L | transcription elongation regulator 1-like | −2.66366 | 0.000149 |
| 227397_at | TPM2 | Tropomyosin 2 (beta) | −2.6637 | 0.0003 |
| 214748_at | RP11-298P3.3 | CG016 | −2.66811 | 0.00416 |
| 205014_at | FGFBP1 | fibroblast growth factor binding protein 1 | −2.66869 | 0.004893 |
| 207547_s_at | FAM107A | family with sequence similarity 107, member A | −2.6697 | 0.000601 |
| 241031_at | NLF1 | Nuclear localized factor 1 | −2.67227 | 0.000255 |
| 222043_at | CLU | clusterin | −2.67255 | 5.19E−06 |
| 209355_s_at | PPAP2B | phosphatidic acid phosphatase type 2B | −2.68598 | 0.000126 |
| 228377_at | KLHL14 | kelch-like 14 (Drosophila) | −2.68712 | 0.007947 |
| 209466_x_at | PTN | pleiotrophin (heparin binding growth factor 8, neurite growth-promoting | −2.6936 | 0.000237 |
| | | factor 1) | | |
| 1565628_at | LRRK1 | Leucine-rich repeat kinase 1 | −2.69411 | 1.38E−05 |
| 209304_x_at | GADD45B | growth arrest and DNA-damage-inducible, beta | −2.69797 | 0.000404 |
| 206710_s_at | EPB41L3 | erythrocyte membrane protein band 4.1-like 3 | −2.69908 | 0.003608 |
| 216236_s_at | SLC2A3 | solute carrier family 2 (facilitated glucose transporter), member 3 | −2.69996 | 0.003025 |
| 214624_at | UPK1A | uroplakin 1A | −2.70307 | 0.006368 |
| 227654_at | LOC200230 | similar to KIAA0386 | −2.72128 | 0.001247 |
| 229910_at | SHE | Src homology 2 domain containing E | −2.73324 | 5.04E−05 |
| 229339_at | — | Transcribed locus | −2.74064 | 0.004022 |
| 233882_s_at | SEMA6D | sema domain, transmembrane domain (TM), and cytoplasmic domain, | −2.75561 | 0.000256 |
| | | (semaphorin) 6D | | |
| 204723_at | SCN3B | sodium channel, voltage-gated, type III, beta | −2.75679 | 0.001359 |
| 229824_at | — | CDNA FLJ45325 fis, clone BRHIP3006717 | −2.75697 | 1.08E−06 |
| 202499_s_at | SLC2A3 | solute carrier family 2 (facilitated glucose transporter), member 3 | −2.76316 | 2.98E−05 |
| 207067_s_at | HDC | histidine decarboxylase | −2.76642 | 1.74E−08 |
| 204420_at | FOSL1 | FOS-like antigen 1 | −2.76741 | 0.006935 |
| 235878_at | TAF1B | TATA box binding protein (TBP)-associated factor, RNA polymerase I, | −2.78562 | 0.002315 |
| | | B, 63 kDa | | |
| 230655_at | — | Homo sapiens, clone IMAGE: 5418468, mRNA | −2.79147 | 0.004077 |
| 244802_at | GLUD1 | glutamate dehydrogenase 1 | −2.79273 | 0.000337 |
| 211343_s_at | COL13A1 | collagen, type XIII, alpha 1 | −2.80324 | 0.000394 |
| 226985_at | FGD5 | FYVE, RhoGEF and PH domain containing 5 | −2.80384 | 1.43E−07 |
| 230746_s_at | STC1 | Stanniocalcin 1 | −2.80435 | 0.002993 |
| 202497_x_at | SLC2A3 | solute carrier family 2 (facilitated glucose transporter), member 3 | −2.81749 | 0.001174 |
| 209074_s_at | FAM107A | family with sequence similarity 107, member A | −2.81792 | 0.006123 |
| 206631_at | PTGER2 | prostaglandin E receptor 2 (subtype EP2), 53 kDa | −2.82069 | 0.000226 |
| 228504_at | — | Transcribed locus, strongly similar to NP_002967.1 sodium channel, | −2.83472 | 0.004664 |
| | | voltage-gated, type VII, alpha; sodium channel, voltage-gated, type VI, | | |
| | | alpha polypeptide; sodium channel, voltage-gated, type VII, alpha | | |
| | | polypeptide; sodium channel, voltage-gated, type VI, alpha [Homo | | |
| | | sapiens] | | |
| 219764_at | FZD10 | frizzled homolog 10 (Drosophila) | −2.83491 | 0.000291 |
| 228045_at | — | MRNA; cDNA DKFZp451K063 (from clone DKFZp451K063) | −2.84183 | 0.001608 |
| 207574_s_at | GADD45B | growth arrest and DNA-damage-inducible, beta | −2.8422 | 4.93E−05 |
| 206236_at | GPR4 | G protein-coupled receptor 4 | −2.84661 | 0.000277 |
| 210764_s_at | CYR61 | cysteine-rich, angiogenic inducer, 61 | −2.87199 | 0.000337 |
| 212226_s_at | PPAP2B | phosphatidic acid phosphatase type 2B | −2.87532 | 0.000394 |
| 242868_at | EPAS1 | Endothelial PAS domain protein 1 | −2.88577 | 0.000304 |
| 230087_at | PRIMA1 | proline rich membrane anchor 1 | −2.89749 | 0.00471 |
| 229337_at | USP2 | ubiquitin specific peptidase 2 | −2.90856 | 0.000273 |
| 231048_at | — | Transcribed locus, moderately similar to XP_498452.1 PREDICTED: | −2.91909 | 0.001324 |
| | | hypothetical protein XP_498452 [Homo sapiens] | | |
| 204622_x_at | NR4A2 | nuclear receptor subfamily 4, group A, member 2 | −2.92412 | 0.001824 |
| 220324_at | C6orf155 | chromosome 6 open reading frame 155 | −2.92715 | 8.77E−05 |
| 209325_s_at | RGS16 | regulator of G-protein signalling 16 | −2.93153 | 0.000665 |
| 227550_at | LOC143381 | hypothetical protein LOC143381 | −2.94142 | 1.04E−06 |
| 39402_at | IL1B | interleukin 1, beta | −2.94181 | 0.001163 |
| 204597_x_at | STC1 | stanniocalcin 1 | −2.94547 | 0.000765 |
| 206359_at | SOCS3 | suppressor of cytokine signaling 3 | −2.9464 | 0.001861 |
| 227099_s_at | LOC387763 | hypothetical LOC387763 | −2.95479 | 3.92E−08 |
| 207542_s_at | AQP1 | aquaporin 1 (Colton blood group) | −2.97642 | 1.11E−05 |
| 216887_s_at | LDB3 | LIM domain binding 3 | −2.98638 | 0.000688 |
| 243296_at | PBEF1 | Pre-B-cell colony enhancing factor 1 | −3.00539 | 0.008773 |
| 206030_at | ASPA | aspartoacylase (Canavan disease) | −3.00627 | 8.89E−06 |
| 225894_at | SYNPO2 | synaptopodin 2 | −3.01942 | 5.84E−07 |
| 209292_at | ID4 | Inhibitor of DNA binding 4, dominant negative helix-loop-helix protein | −3.02656 | 0.000422 |
| 227307_at | TSPAN18 | Tetraspanin 18 | −3.02788 | 7.75E−06 |
| 231947_at | MYCT1 | myc target 1 | −3.02915 | 7.77E−07 |
| 216598_s_at | CCL2 | chemokine (C-C motif) ligand 2 | −3.03004 | 0.006846 |
| 222911_s_at | CXorf36 | chromosome X open reading frame 36 | −3.04217 | 4.54E−05 |
| 229201_at | — | Full-length cDNA clone CS0DF014YC15 of Fetal brain of Homo | −3.05567 | 0.000745 |
| | | sapiens (human) | | |
| 235898_at | — | Transcribed locus | −3.06118 | 0.001323 |
| 201787_at | FBLN1 | fibulin 1 | −3.06953 | 0.007431 |
| 209469_at | GPM6A | glycoprotein M6A | −3.07588 | 0.006286 |
| 229491_at | LOC133308 | hypothetical protein BC009732 | −3.08197 | 3.50E−06 |
| 235291_s_at | FLJ32255 /// | hypothetical protein LOC643977 /// hypothetical protein LOC648075 | −3.08206 | 0.00598 |
| | LOC648075 | | | |
| 227870_at | NOPE | likely ortholog of mouse neighbor of Punc E11 | −3.08451 | 0.00404 |
| 227819_at | LGR6 | leucine-rich repeat-containing G protein-coupled receptor 6 | −3.08657 | 0.000349 |
| 205295_at | CKMT2 | creatine kinase, mitochondrial 2 (sarcomeric) | −3.09439 | 0.006811 |
| 219046_s_at | PKNOX2 | PBX/knotted 1 homeobox 2 | −3.12291 | 6.25E−05 |
| 204955_at | SRPX | sushi-repeat-containing protein, X-linked | −3.1266 | 0.001089 |
| 207496_at | MS4A2 | membrane-spanning 4-domains, subfamily A, member 2 (Fc fragment | −3.13502 | 2.00E−05 |
| | | of IgE, high affinity I, receptor for; beta polypeptide) | | |
| 1554190_s_at | C10orf81 | chromosome 10 open reading frame 81 | −3.13725 | 0.002988 |
| 222171_s_at | PKNOX2 | PBX/knotted 1 homeobox 2 | −3.14666 | 0.008142 |
| 222124_at | HIF3A | hypoxia inducible factor 3, alpha subunit | −3.14891 | 0.001211 |
| 229178_at | LOC145786 | hypothetical protein LOC145786 | −3.16514 | 1.72E−07 |
| 214038_at | CCL8 | chemokine (C-C motif) ligand 8 | −3.17308 | 0.000807 |
| 235718_at | SRL | sarcalumenin | −3.18431 | 0.002671 |
| 1568768_s_at | BRE | brain and reproductive organ-expressed (TNFRSF1A modulator) | −3.21394 | 7.99E−05 |
| 229306_at | NLF2 | Nuclear localized factor 2 | −3.216 | 5.88E−05 |
| 204273_at | EDNRB | endothelin receptor type B | −3.21745 | 4.36E−06 |
| 227769_at | — | Transcribed locus, strongly similar to NP_061844.1 G protein-coupled | −3.21837 | 0.001387 |
| | | receptor 27; super conserved receptor expressed in brain 1 [Homo | | |
| | | sapiens] | | |
| 214769_at | — | Clone 23809 mRNA sequence | −3.22734 | 0.006563 |
| 201531_at | ZFP36 | zinc finger protein 36, C3H type, homolog (mouse) | −3.23194 | 0.000594 |
| 232313_at | TMEM132C | transmembrane protein 132C | −3.24192 | 9.99E−06 |
| 221667_s_at | HSPB8 | heat shock 22 kDa protein 8 | −3.24712 | 0.005706 |
| 217997_at | PHLDA1 | pleckstrin homology-like domain, family A, member 1 | −3.24888 | 2.72E−06 |
| 237390_at | ADRA1A | Adrenergic, alpha-1A-, receptor | −3.24911 | 0.00065 |
| 208335_s_at | DARC | Duffy blood group, chemokine receptor | −3.26469 | 0.000362 |
| 217999_s_at | PHLDA1 | pleckstrin homology-like domain, family A, member 1 | −3.27888 | 7.41E−05 |
| 205392_s_at | CCL14 /// CCL15 | chemokine (C-C motif) ligand 14 /// chemokine (C-C motif) ligand 15 | −3.28976 | 2.25E−05 |
| 216248_s_at | NR4A2 | nuclear receptor subfamily 4, group A, member 2 | −3.30311 | 0.001224 |
| 214043_at | PTPRD | Protein tyrosine phosphatase, receptor type, D | −3.31033 | 0.000964 |
| 225061_at | DNAJA4 | DnaJ (Hsp40) homolog, subfamily A, member 4 | −3.31148 | 0.006936 |
| 241369_at | ADAMTS9 | ADAM metallopeptidase with thrombospondin type 1 motif, 9 | −3.31542 | 0.0075 |
| 228698_at | SOX7 | SRY (sex determining region Y)-box 7 | −3.32626 | 2.75E−06 |
| 208078_s_at | SNF1LK | SNF1-like kinase /// SNF1-like kinase | −3.32973 | 0.008038 |
| 1560425_s_at | PTPRD | Protein tyrosine phosphatase, receptor type, D | −3.33439 | 0.001335 |
| 233059_at | — | CDNA: FLJ22731 fis, clone HSI15841 | −3.3401 | 3.76E−05 |
| 202912_at | ADM | adrenomedullin | −3.34707 | 0.006209 |
| 227971_at | NRK | Nik related kinase | −3.35945 | 0.004506 |
| 242931_at | — | — | −3.36185 | 0.000789 |
| 206510_at | SIX2 | sine oculis homeobox homolog 2 (Drosophila) | −3.36603 | 0.001853 |
| 239671_at | — | CDNA FLJ31085 fis, clone IMR321000037 | −3.36788 | 0.009347 |
| 63305_at | PKNOX2 | PBX/knotted 1 homeobox 2 | −3.37429 | 0.000984 |
| 219857_at | C10orf81 | chromosome 10 open reading frame 81 | −3.3784 | 0.000575 |
| 223497_at | KIAA1411 | KIAA1411 | −3.37942 | 0.005858 |
| 233503_at | SOHLH2 | spermatogenesis and oogenesis specific basic helix-loop-helix 2 | −3.40397 | 0.000849 |
| 241986_at | BMPER | BMP binding endothelial regulator | −3.40551 | 0.001417 |
| 201170_s_at | BHLHB2 | basic helix-loop-helix domain containing, class B, 2 | −3.43749 | 0.000989 |
| 209189_at | FOS | v-fos FBJ murine osteosarcoma viral oncogene homolog | −3.45049 | 0.006621 |
| 206698_at | XK | X-linked Kx blood group (McLeod syndrome) | −3.45115 | 0.008127 |
| 205249_at | EGR2 | early growth response 2 (Krox-20 homolog, Drosophila) | −3.46001 | 1.02E−05 |
| 236448_at | UNC5A | unc-5 homolog A (C. elegans) | −3.50241 | 0.002055 |
| 222088_s_at | SLC2A3 | solute carrier family 2 (facilitated glucose transporter), member 3 | −3.50635 | 0.000104 |
| 213362_at | PTPRD | Protein tyrosine phosphatase, receptor type, D | −3.5494 | 0.002396 |
| 238175_at | ACAD8 | Acyl-Coenzyme A dehydrogenase family, member 8 | −3.5532 | 0.007858 |
| 229544_at | — | CDNA clone IMAGE: 4791887 /// MRNA; cDNA DKFZp564C0762 (from | −3.55658 | 0.001429 |
| | | clone DKFZp564C0762) | | |
| 202498_s_at | SLC2A3 | solute carrier family 2 (facilitated glucose transporter), member 3 | −3.56992 | 0.000151 |
| 232298_at | LOC401093 | hypothetical LOC401093 | −3.57569 | 4.67E−06 |
| 228807_at | — | — | −3.61478 | 0.001427 |
| 232305_at | HMGCLL1 | 3-hydroxymethyl-3-methylglutaryl-Coenzyme A lyase-like 1 | −3.63484 | 0.006952 |
| 213992_at | COL4A6 | collagen, type IV, alpha 6 | −3.64493 | 0.00015 |
| 239572_at | — | Transcribed locus | −3.64998 | 0.00349 |
| 205067_at | IL1B | interleukin 1, beta | −3.65032 | 8.85E−05 |
| 206049_at | SELP | selectin P (granule membrane protein 140 kDa, antigen CD62) | −3.66965 | 5.39E−07 |
| 227697_at | SOCS3 | suppressor of cytokine signaling 3 | −3.68467 | 8.93E−05 |
| 219949_at | LRRC2 | leucine rich repeat containing 2 | −3.68643 | 0.000357 |
| 238178_at | — | — | −3.69978 | 7.70E−08 |
| 203887_s_at | THBD | thrombomodulin | −3.72463 | 5.85E−08 |
| 209470_s_at | GPM6A | glycoprotein M6A | −3.8136 | 0.002777 |
| 202672_s_at | ATF3 | activating transcription factor 3 | −3.84116 | 0.002766 |
| 204621_s_at | NR4A2 | nuclear receptor subfamily 4, group A, member 2 | −3.86017 | 9.37E−05 |
| 230943_at | — | Transcribed locus | −3.89286 | 1.83E−06 |
| 220724_at | FLJ21511 | hypothetical protein FLJ21511 | −3.8938 | 0.00555 |
| 203888_at | THBD | thrombomodulin | −3.93537 | 1.46E−07 |
| 1562275_at | ADAMTS9 | ADAM metallopeptidase with thrombospondin type 1 motif, 9 | −3.95623 | 0.000438 |
| 207978_s_at | NR4A3 | nuclear receptor subfamily 4, group A, member 3 | −3.97338 | 0.000108 |
| 205132_at | ACTC | actin, alpha, cardiac muscle | −4.02649 | 0.000249 |
| 209583_s_at | CD200 | CD200 molecule | −4.29201 | 1.93E−11 |
| 205430_at | BMP5 | bone morphogenetic protein 5 | −4.32133 | 0.003025 |
| 201693_s_at | EGR1 | early growth response 1 | −4.40738 | 0.001462 |
| 205266_at | LIF | leukemia inhibitory factor (cholinergic differentiation factor) | −4.41738 | 5.45E−07 |
| 1552727_s_at | ADAMTS17 | ADAM metallopeptidase with thrombospondin type 1 motif, 17 | −4.4379 | 4.00E−09 |
| 237252_at | THBD | thrombomodulin | −4.45059 | 2.09E−10 |
| 209457_at | DUSP5 | dual specificity phosphatase 5 | −4.49186 | 3.40E−06 |
| 209959_at | NR4A3 | nuclear receptor subfamily 4, group A, member 3 | −4.57847 | 0.000138 |
| 215078_at | SOD2 | superoxide dismutase 2, mitochondrial | −4.63337 | 0.002228 |
| 230537_at | — | — | −4.78938 | 0.000383 |
| 206932_at | CH25H | cholesterol 25-hydroxylase | −4.86764 | 1.36E−08 |
| 236203_at | HLA-DRB1 | Major histocompatibility complex, class II, DR beta 1 | −5.32987 | 0.006402 |
| 206211_at | SELE | selectin E (endothelial adhesion molecule 1) | −5.55668 | 1.42E−07 |
| 202768_at | FOSB | FBJ murine osteosarcoma viral oncogene homolog B | −5.60327 | 0.003448 |
| 206115_at | EGR3 | early growth response 3 | −5.69246 | 2.19E−12 |
| 205207_at | IL6 | interleukin 6 (interferon, beta 2) | −5.72771 | 1.34E−05 |
| 220129_at | SOHLH2 | spermatogenesis and oogenesis specific basic helix-loop-helix 2 | −5.77561 | 0.000755 |
| 208016_s_at | AGTR1 | angiotensin II receptor, type 1 | −6.32205 | 6.67E−11 |
| 205357_s_at | AGTR1 | angiotensin II receptor, type 1 | −6.35681 | 5.31E−08 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An in vitro method for identifying a subject as having or not having prostate cancer, comprising:
(a) providing a prostate tissue sample from said subject;
(b) measuring the level of expression for prostate cancer signature genes in said sample;
(c) comparing said measured expression levels to reference expression levels for said prostate cancer signature genes; and
(d) if said measured expression levels are significantly greater or less than said reference expression levels, identifying said subject as having prostate cancer, and if said measured expression levels are not significantly greater or less than said reference expression levels, identifying said subject as not having prostate cancer.

2. The method of claim 1, wherein said prostate tissue sample does not include tumor cells.

3. The method of claim 1, wherein said prostate tissue sample includes tumor cells and stromal cells.

4. The method of claim 1, wherein said prostate cancer signature genes are selected from the genes listed in Table 3 or Table 4 herein.

5. The method of claim 1, comprising determining whether measured expression levels for ten or more prostate cancer signature genes are significantly greater or less than reference expression levels for said ten or more prostate cancer signature genes, and classifying said subject as having prostate cancer that is likely to relapse if said measured expression levels are significantly greater or less than said reference expression levels, or classifying said subject as having prostate cancer not likely to relapse if said measured expression levels are not significantly greater or less than said reference expression levels.

6. The method of claim 5, wherein said ten or more prostate cancer signature genes are selected from the genes listed in Table 3 or Table 4 herein.

7. The method of claim 1, comprising determining whether measured expression levels for twenty or more prostate cancer signature genes are significantly greater or less than reference expression levels for said twenty or more prostate cancer signature genes, and classifying said subject as having prostate cancer that is likely to relapse if said measured expression levels are significantly greater or less than said reference expression levels, or classifying said subject as having prostate cancer not likely to relapse if said measured expression levels are not significantly greater or less than said reference expression levels.

8. The method of claim 7, wherein said twenty or more prostate cancer signature genes are selected from the genes listed in Table 3 or Table 4 herein.

9. A method for determining the prognosis of a subject diagnosed as having prostate cancer, comprising:
(a) providing a prostate tissue sample from said subject;
(b) measuring the level of expression for prostate cancer signature genes in said sample;
(c) comparing said measured expression levels to reference expression levels for said prostate cancer signature genes; and
(d) if said measured expression levels are not significantly greater or less than said reference expression levels, identifying said subject as having a relatively better prognosis than if said measured expression levels are significantly greater or less than said reference expression levels, or if said measured expression levels are significantly greater or less than said reference expression levels, identifying said subject as having a relatively worse prognosis than if said measured expression levels are not significantly greater or less than said reference expression levels.

10. The method of claim 9, wherein said prostate tissue sample does not include tumor cells.

11. The method of claim 9, wherein said prostate tissue sample includes tumor cells and stromal cells.

12. The method of claim 9, wherein said prostate cancer signature genes are selected from the genes listed in Table 8A or 8B herein.

13. A method for identifying a subject as having or not having prostate cancer, comprising:
(a) providing a prostate tissue sample from said subject, wherein said sample comprises prostate stromal cells;
(b) measuring expression levels for one or more genes in said stromal cells, wherein said one or more genes are prostate cancer signature genes;
(c) comparing said measured expression levels to reference expression levels for said one or more genes, wherein said reference expression levels are determined in stromal cells from non-cancerous prostate tissue; and
(d) if said measured expression levels are significantly greater or less than said reference expression levels, identifying said subject as having prostate cancer, and if said measured expression levels are not significantly greater or less than said reference expression levels, identifying said subject as not having prostate cancer.

14. The method of claim 13, wherein said prostate tissue sample does not include tumor cells.

15. The method of claim 13, wherein said prostate tissue sample includes tumor cells and stromal cells.

16. The method of claim 13, wherein said prostate cancer signature genes are selected from the genes listed in Table 3 or Table 4 herein.

17. A method for determining a prognosis for a subject diagnosed as having prostate cancer, comprising:
(a) providing a prostate tissue sample from said subject, wherein said sample comprises prostate stromal cells;
(b) measuring expression levels for one or more genes in said stromal cells, wherein said one or more genes are prostate cancer signature genes;
(c) comparing said measured expression levels to reference expression levels for said one or more genes, wherein said reference expression levels are determined in stromal cells from non-cancerous prostate tissue; and
(d) if said measured expression levels are not significantly greater or less than said reference expression levels, identifying said subject as having a relatively better prognosis than if said measured expression levels are significantly greater or less than said reference expression levels, or if said measured expression levels are significantly greater or less than said reference expression levels, identifying said subject as having a relatively worse prognosis than if said measured expression levels are not significantly greater or less than said reference expression levels.

18. The method of claim 17, wherein said prostate tissue sample does not include tumor cells.

19. The method of claim 17, wherein said prostate tissue sample includes tumor cells and stromal cells.

20. The method of claim 17, wherein said prostate cancer signature genes are selected from the genes listed in Table 3 or Table 4 herein.

21. A method for identifying a subject as having or not having prostate cancer, comprising:
(a) providing a prostate tissue sample from said subject;
(b) measuring expression levels for one or more prostate cell-type predictor genes in said sample;
(c) determining the percentages of tissue types in said sample based on said measured expression levels;
(d) measuring expression levels for one more prostate cancer signature genes in said sample;
(e) determining a classifier based on said percentages of tissue types and said measured expression levels; and
(f) if said classifier falls into a predetermined range of prostate cancer classifiers, identifying said subject as having prostate cancer, or if said classifier does not fall into said predetermined range, identifying said subject as not having prostate cancer.

22. The method of claim 18, wherein steps (b) and (d) are carried out simultaneously.

23. A method for determining a prognosis for a subject diagnosed with and treated for prostate cancer, comprising:
(a) providing a prostate tissue sample from said subject;
(b) measuring expression levels for one or more prostate tissue predictor genes in said sample;
(c) determining the percentages of tissue types in said sample based on said measured expression levels;
(d) measuring expression levels for one more prostate cancer signature genes in said sample;
(e) determining a classifier based on said percentages of tissue types and said measured expression levels; and
(f) if said classifier falls into a predetermined range of prostate cancer relapse classifiers, identifying said subject as being likely to relapse, or if said classifier does not fall into said predetermined range, identifying said subject as not being likely to relapse.

24. The method of claim 23, wherein steps (b) and (d) are carried out simultaneously.

25. A method for identifying the proportion of two or more tissue types in a tissue sample, comprising:
(a) using a set of other samples of known tissue proportions from a similar anatomical location as said tissue sample in an animal or plant, wherein at least two of said other samples do not contain the same relative content of each of the two or more cell types;
(b) measuring overall levels of one or more gene expression or protein analytes in each of said other samples;
(c) determining the regression relationship between the relative proportion of each tissue type and the measured overall levels of each gene expression or protein analyte in said other samples;
(d) selecting one or more analytes that correlate with tissue proportions in said other samples;
(e) measuring overall levels of one or more of said analytes in step (d) in said tissue sample;
(f) matching the level of each analyte in said tissue sample with the level of said analyte in step (d) to determine the predicted proportion of each tissue type in said tissue sample; and
(g) selecting among predicted tissue proportions for said tissue sample obtained in step
(0 using either the median or average proportions of all the estimates.

26. The method of claim 25, wherein said tissue sample contains cancer cells.

27. The method of claim 26, wherein said cancer is prostate cancer.

28. A method for comparing the levels of two or more analytes predicted by one or more methods to be associated with a change in a biological phenomenon in two sets of data each containing more than one measured sample, comprising:
(a) selecting only analytes that are assayed in both sets of data;
(b) ranking said analytes in each set of data using a comparative method such as the highest probability or lowest false discovery rate associated with the change in the biological phenomenon;
(c) comparing a set of analytes in each ranked list in step (b) with each other, selecting those that occur in both lists, and determining the number of analytes that occur in both lists and show a change in level associated with the biological phenomenon that is in the same direction; and
(d) calculating a concordance score based on the probability that said number of comparisons would show the observed number of change in the same direction, at random.

29. The method of claim 28, wherein in step (a) the length of each list is varied to determine the maximum concordance score for the two ranked lists.
